(19) United States
(12) Patent Application Publication
(10) Pub. No.: US 2025/0154141 A1
(43) Pub. Date: May 15, 2025

(54) BCL-XL/BCL-2 DUAL DEGRADERS FOR TREATMENT OF CANCERS

(71) Applicant: University of Florida Research Foundation, Incorporated, Gainesville, FL (US)

(72) Inventors: Guangrong Zheng, Gainesville, FL (US); Daohong Zhou, Gainesville, FL (US); Wanyi Hu, Gainesville, FL (US); Dinesh Thummuri, Gainesville, FL (US); Peiyi Zhang, Gainesville, FL (US); Pratik Pal, Gainesville, FL (US); Dongwen Lyu, Gainesville, FL (US); Yaxia Yuan, Gainesville, FL (US)

(73) Assignee: University of Florida Research Foundation, Incorporated, Gainesville, FL (US)

(21) Appl. No.: 18/717,509

(22) PCT Filed: Dec. 8, 2022
(86) PCT No.: PCT/US2022/052228

§ 371 Date: Jun. 7, 2024

Related U.S. Application Data
(60) Provisional application No. 63/287,962, filed on Dec. 9, 2021.

(51) Int. Cl. C07D 417/14; A61K 31/496; A61K 31/5377; A61K 31/5386; A61K 31/553; A61K 45/06; A61P 35/02; C07D 413/14; C07D 471/04; C07D 471/08; C07D 491/107; C07D 498/08

(52) U.S. Cl. CPC C07D 417/14; A61K 31/496; A61K 31/5377; A61K 31/5386; A61K 31/553; A61K 45/06; A61P 35/02; C07D 413/14; C07D 471/04; C07D 471/08; C07D 491/107; C07D 498/08

(57) ABSTRACT

Provided herein are compounds (e.g., compounds of Formula (I) or Formula (II)), their mechanism of action, and methods of modulating proliferation activity, and methods of treating diseases and disorders using the compounds provided herein (e.g., compounds of Formula (I) or Formula (II)).

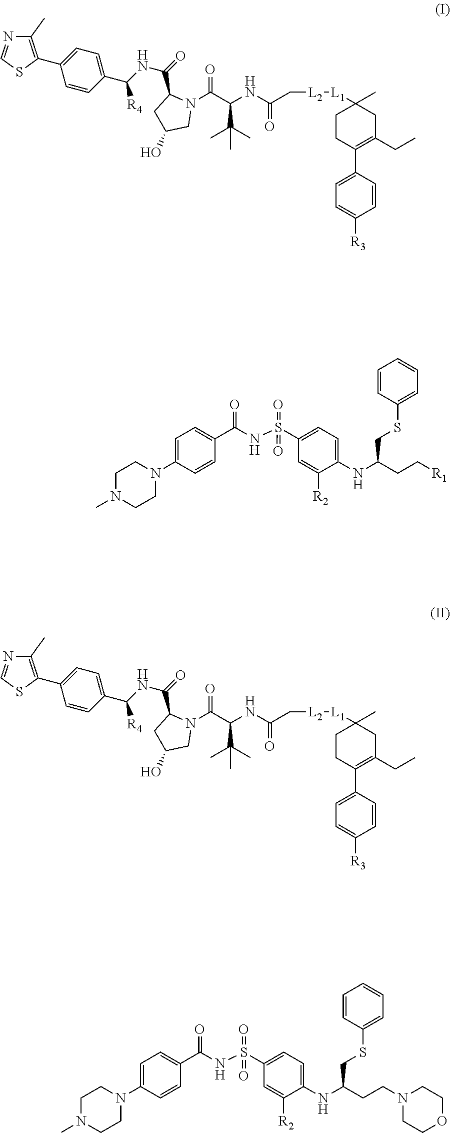

### RELATED APPLICATIONS

4 This patent application claims priority to U.S. Provisional Patent Application No. 63/287,962, filed Dec. 9, 2021, which is incorporated herein by reference in its entirety.

### GOVERNMENT SUPPORT INFORMATION

This invention was made with government support under Grant Nos. R01 CA242003 and R01 CA241191 awarded by the National Institutes of Health. The government has certain rights in the invention.

### BACKGROUND

The B-cell lymphoma 2 (Bcl-2) protein family, consisting of pro- and anti-apoptotic members, plays a critical role in determining cell fate through regulation of the intrinsic apoptosis pathway. The anti-apoptotic Bcl-2 family proteins, such as Bcl-2, Bcl-xL, Bcl-w, and Mcl-1, are upregulated in many cancers and associated with tumor initiation, progression, and resistance to chemo- and targeted therapies. Thus, these anti-apoptotic Bcl-2 proteins are attractive targets for the development of novel anti-cancer agents (Lessene et al., Nat Rev Drug Discov 7: 989-1000, 2008; Vogler et al., Cell Death Differ 2009; 16: 360-367; Delbridge et al., Nat Rev Cancer 16: 99-109, 2016). Numerous Bcl-2 small molecule inhibitors have been reported (Bajwa et al., Expert Opin Ther Patents 22:37-55, 2012; Vogler, Adv Med. 1-14, 2014; Ashkenazi et al., 16: 273-284, 2017). The following are some of the Bcl-2 small molecule inhibitors that have been investigated at various stages of drug development: ABT-737 (US20070072860), navitoclax (ABT-263, WO2009155386), venetoclax (ABT-199, WO2010138588), obatoclax (GX 15-070, WO2004106328), (−)-gossypol (AT-101, WO2002097053), sabutoclax (BI-97C1, WO2010120943), TW-37 (WO2006023778), BM-1252 (APG-1252), and A-1155463 (VV02010080503).

Venetoclax, a selective Bcl-2 inhibitor, was approved by the FDA in 2016 for the treatment of chronic lymphocytic leukemia (CLL) with 17-p deletion. Venetoclax was designed to have high selectivity for Bcl-2 over Bcl-xL to avoid the on-target platelet toxicity (Souers et al., Nat Med 19: 202-208, 2013). Platelets depend on Bcl-xL to maintain their viability, therefore dose-limiting thrombocytopenia has been observed in animals and/or humans treated with ABT-(Schoenwaelder et al., Blood 118: 1663-1674, 2011), ABT-263 (Tse et al., Cancer Res 68: 3421-3428, 2008; Roberts et al., Bri J Haematol 170: 669-678, 2015), BM-1197 (Bai et al., PLoS ONE 9:e99404, 2014), or A-1155463 (Tao et al., ACS Med Chem Lett 5:1088-1093, 2014), due to their inhibition of Bcl-xL. However, many CLL patients are resistant to venetoclax (Roberts et al., N Engl J Med 374: 311-322, 2016) and upregulation of Bcl-xL by microenvironmental survival signals has been identified as the major component accountable for the resistance, consistent with the high efficacy of Bcl-2/Bcl-xL dual inhibitor ABT-263 in killing venetoclax resistant CLL cells (Oppermann et al., Blood 128: 934-947, 2016). In addition, Bcl-xL is generally more frequently overexpressed than Bcl-2 in solid tumors. Importantly, promising results have been documented from preclinical and clinical studies of ABT-263, as a single-agent or in combination with other antitumor agents, against several solid and hematologic malignancies (Delbridge et al., Nat Rev Cancer 16: 99-109, 2016). Therefore, it is highly desirable to develop a strategy that can retain the antitumor versatility and efficacy of the Bcl-xL/Bcl-2 dual inhibitors, while sparing their on-target platelet toxicity.

Thus, there is a need to develop compounds that can retain the antitumor versatility and efficacy of the Bcl-xL/Bcl-2 dual inhibitors, while avoiding their on-target platelet toxicity.

### SUMMARY OF THE INVENTION

Provided herein are compounds (e.g., compounds of Formula (I) or Formula (II)), their mechanism of action, and methods of modulating proliferation activity, and methods of treating diseases and disorders using the compounds provided herein (e.g., compounds of Formula (I) or Formula (II)). In one aspect, the disease or disorder is cancer. In another aspect, the cancer is a Bcl-2-mediated cancer.

In another aspect, provided herein is a compound of Formula (I), or a pharmaceutically acceptable salt, hydrate, solvate, or prodrug thereof:

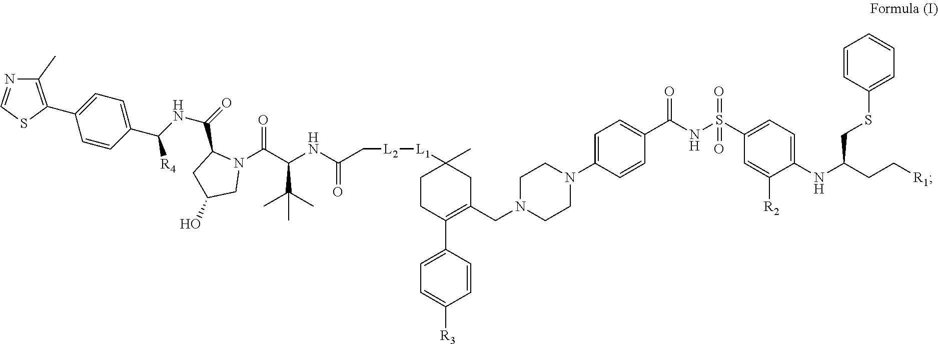

wherein R₁ is

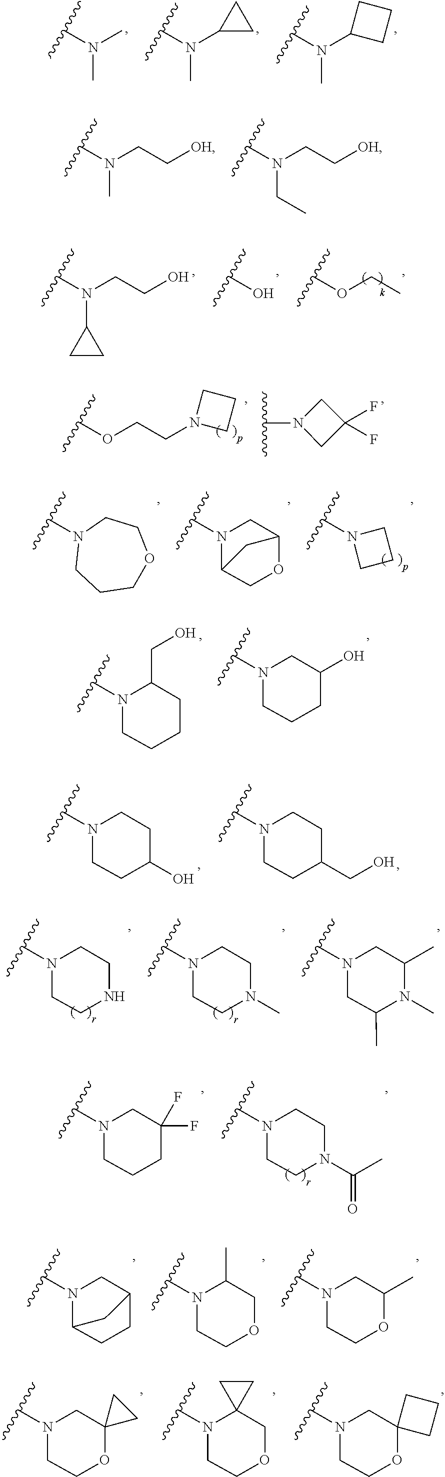

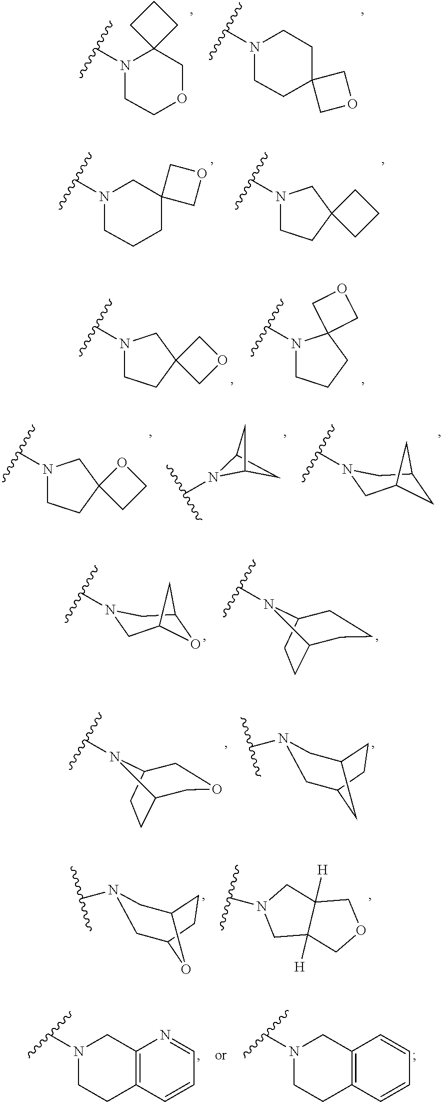

R₂ is NO₂, SO₂CF₃, or SO₂CF₂Cl;
R₃ is Cl, F, CF₂H, CFH₂ or CF₃;
R₄ is H or CH₃;
L₁ is

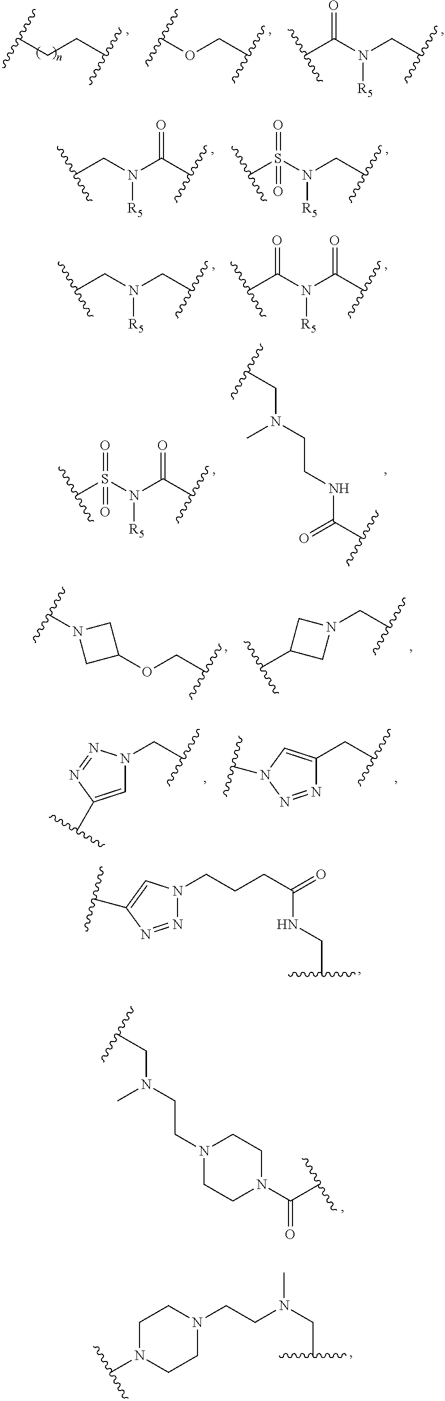

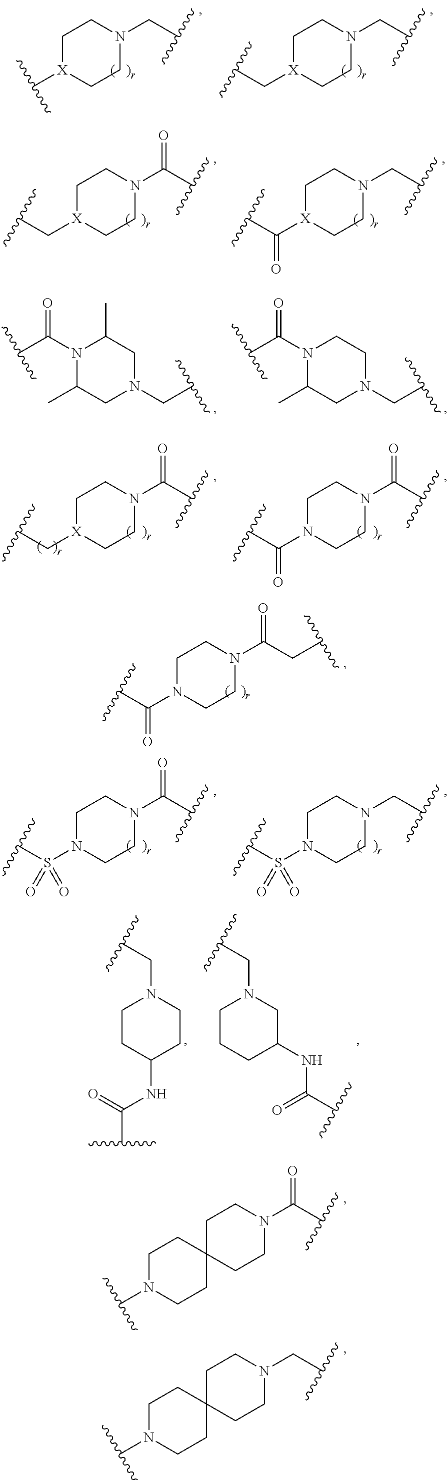

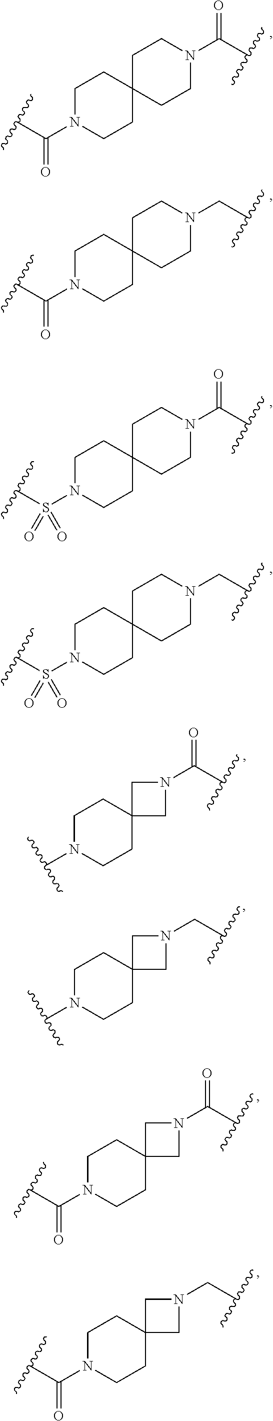

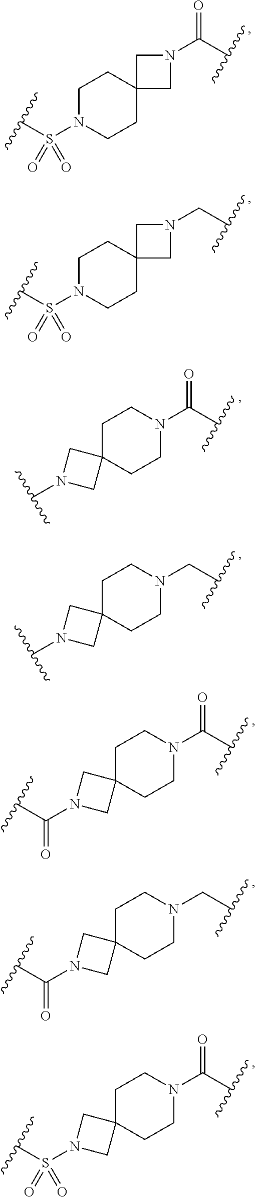

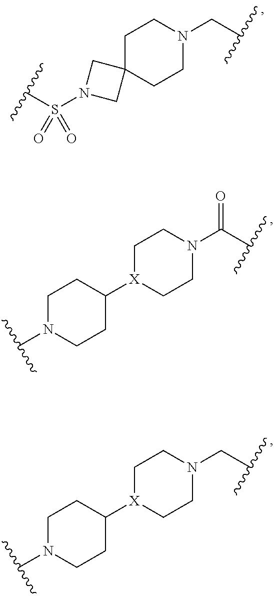

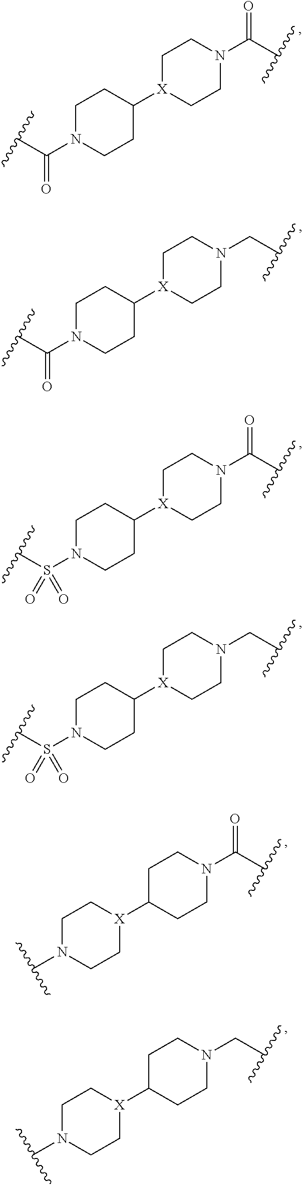

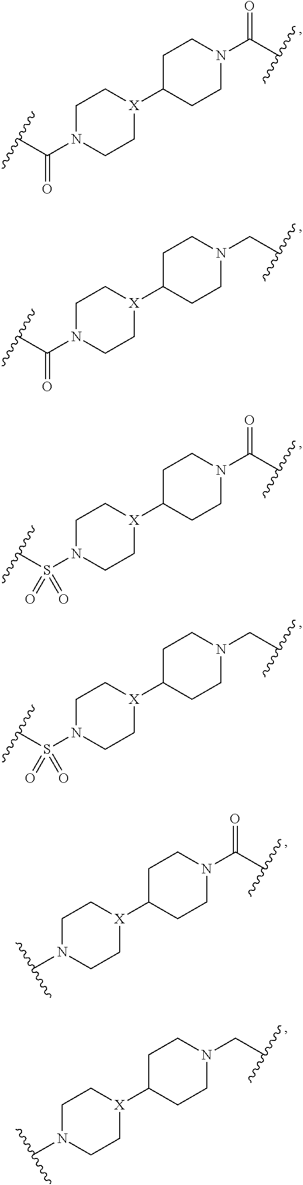

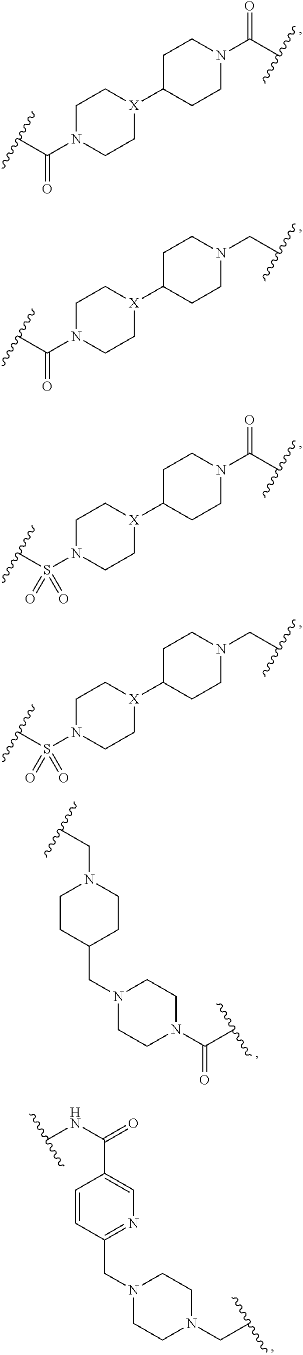

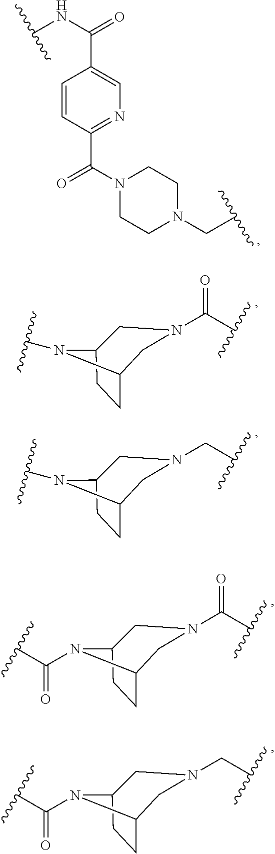

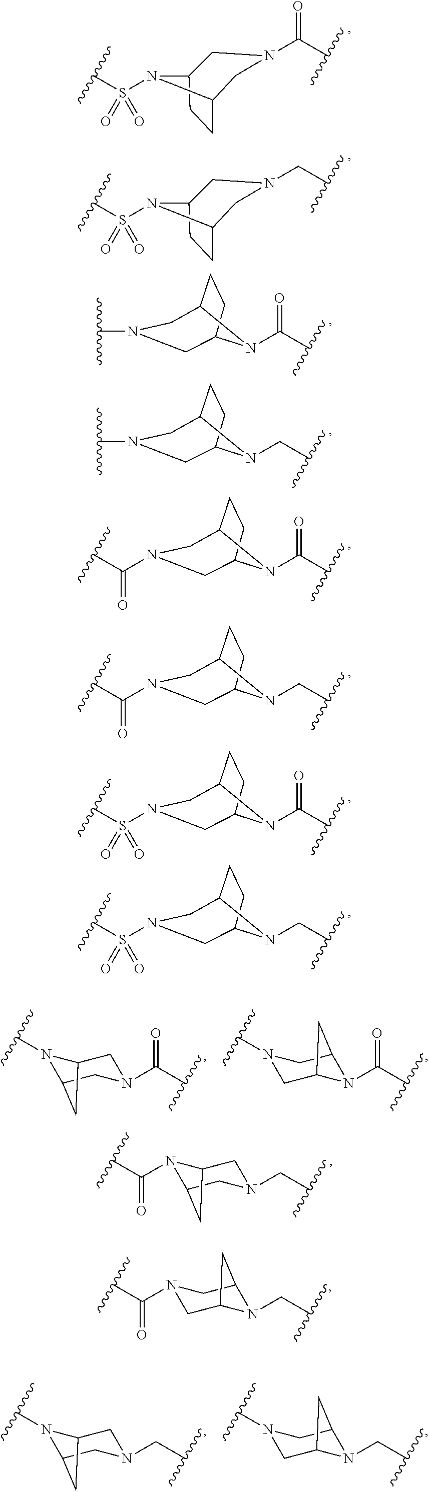

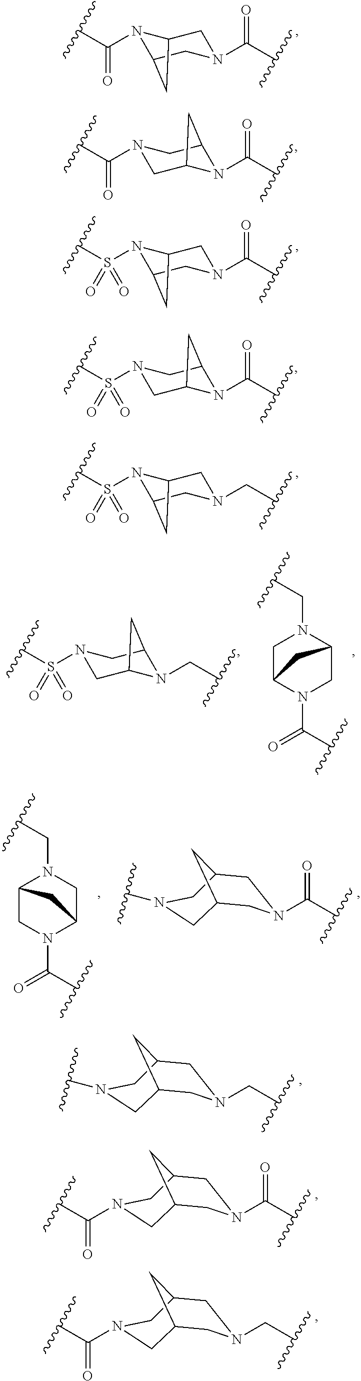

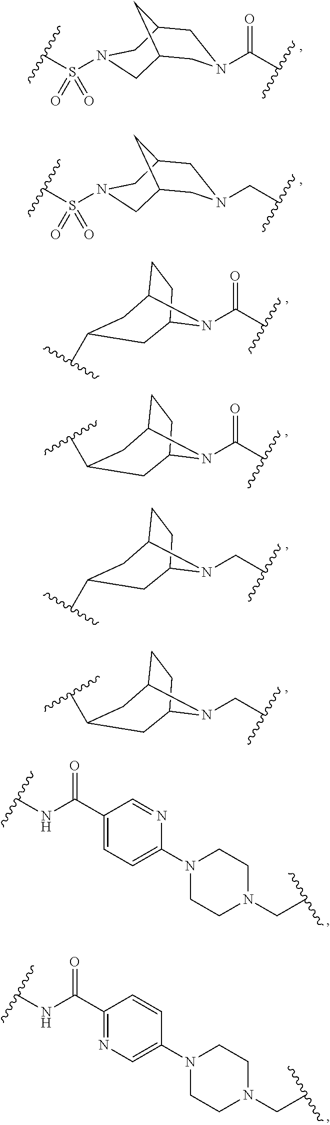

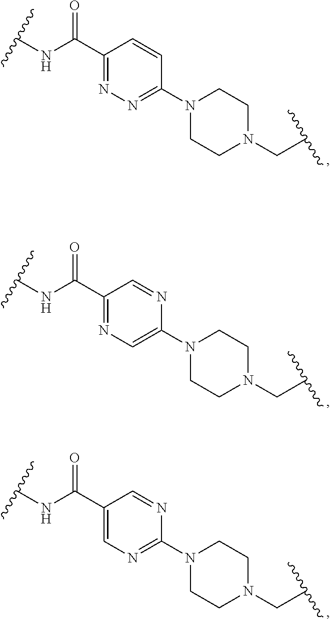 [SMILES: *CN1CCN(c2ccc(C(=O)N*)nn2)CC1 ; *CN1CCN(c2cnc(C(=O)N*)cn2)CC1 ; *CN1CCN(c2ncc(C(=O)N*)cn2)CC1]

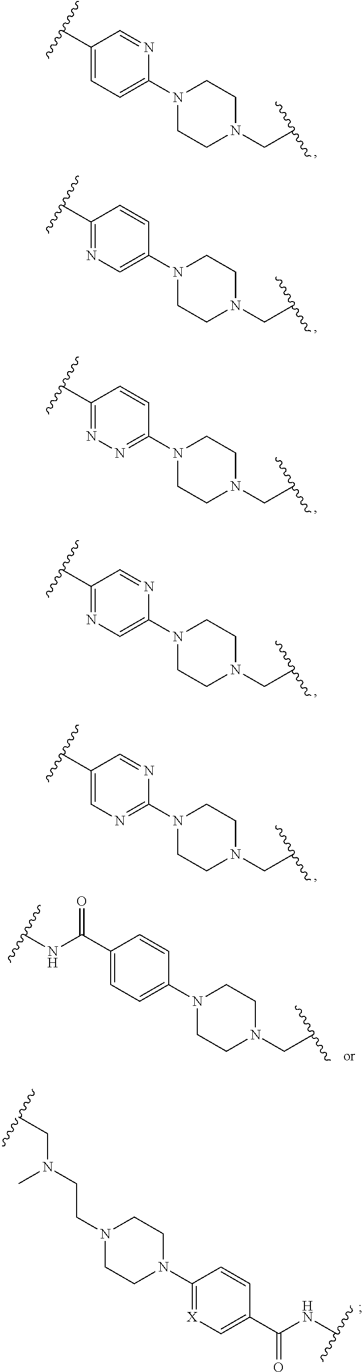

L₂ is a bond,

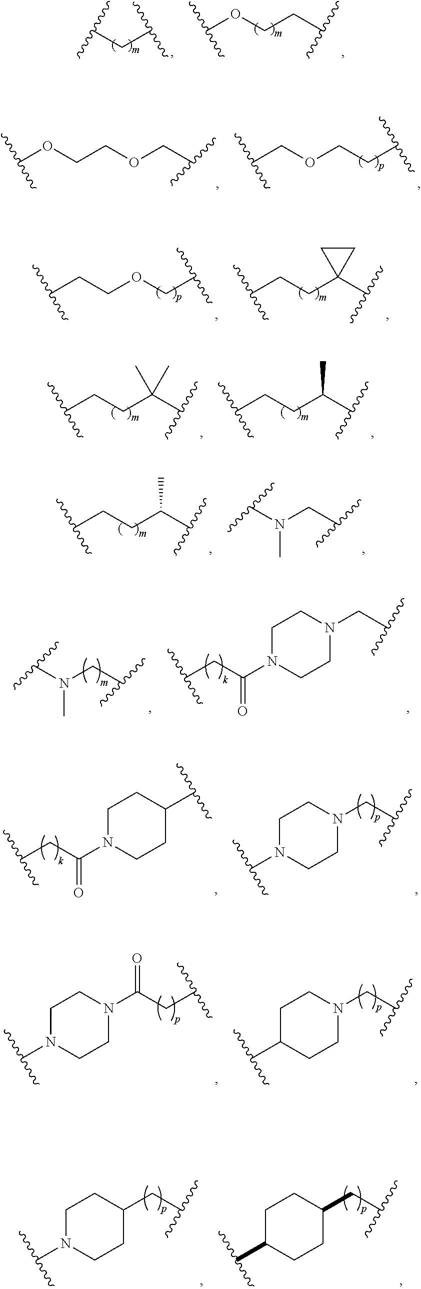

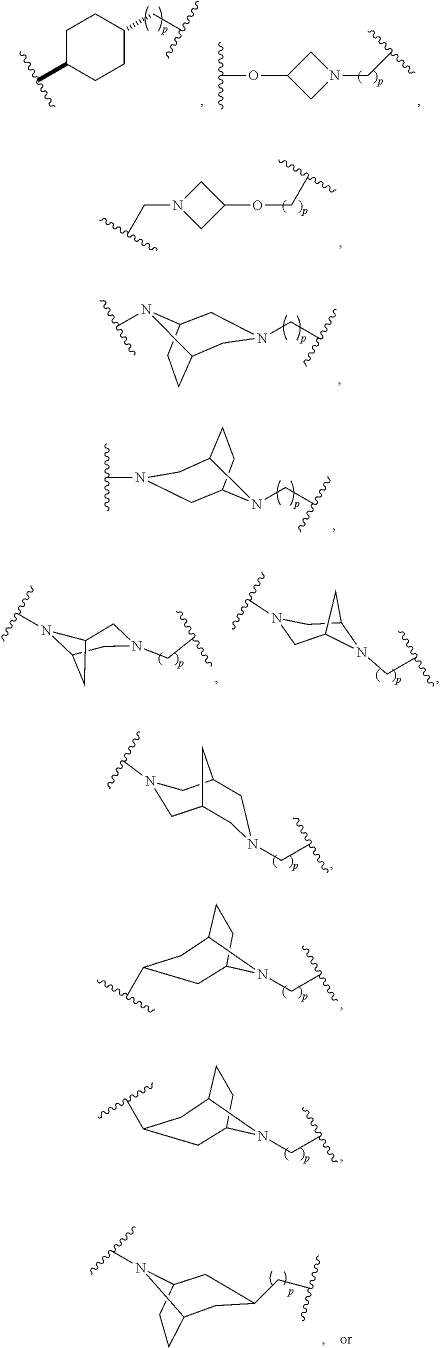

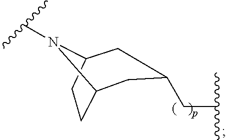

each X is independently CH or N
each k is independently 0, 1, 2, 3, 4, 5, or 6;
each m is independently 2, 3, 4, 5, 6, or 7;
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl.

In another aspect, the compound of Formula (I), or a pharmaceutically acceptable salt, hydrate, solvate, or prodrug thereof, is of Formula (I-A):

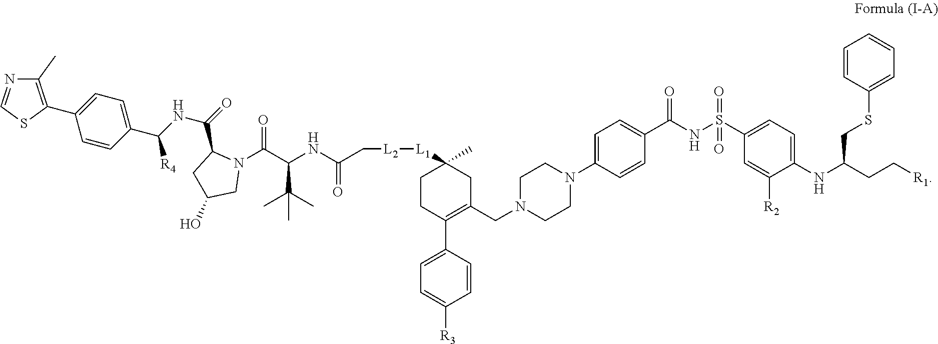

In another aspect, the compound of Formula (I), or a pharmaceutically acceptable salt, hydrate, solvate, or prodrug thereof, is of Formula (I-B):

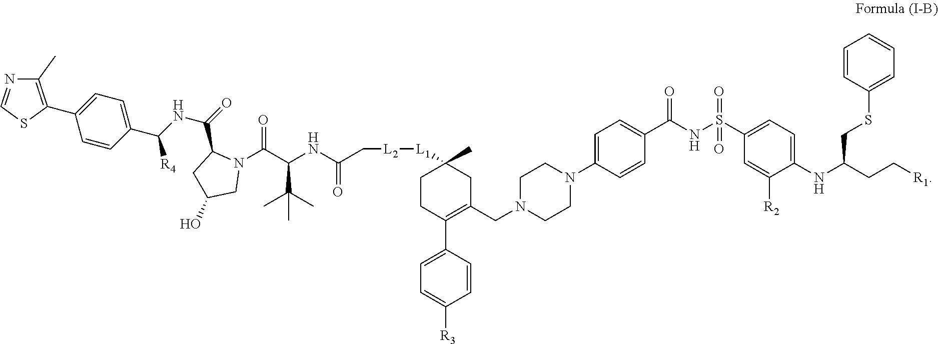

In one aspect, R₁ is

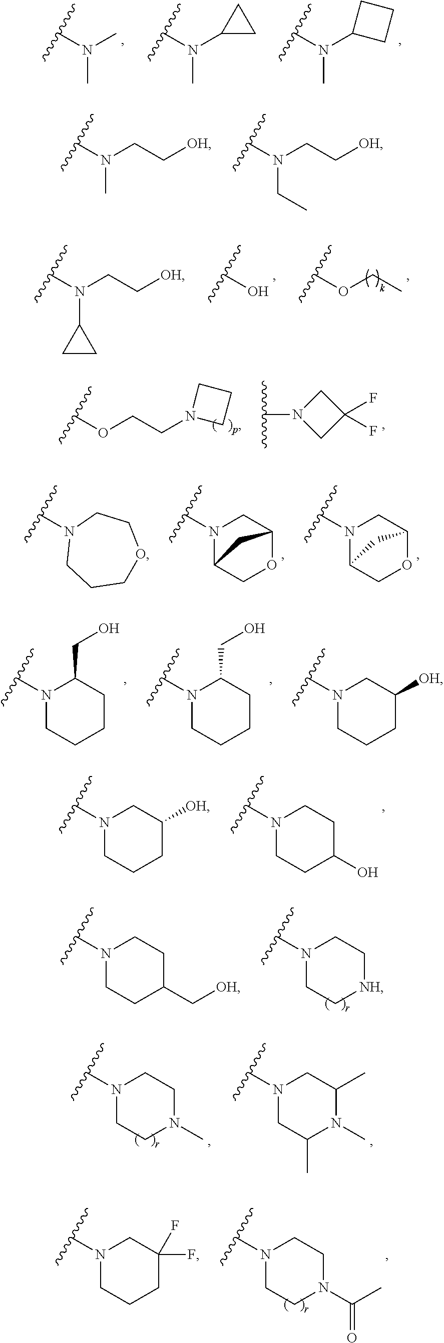

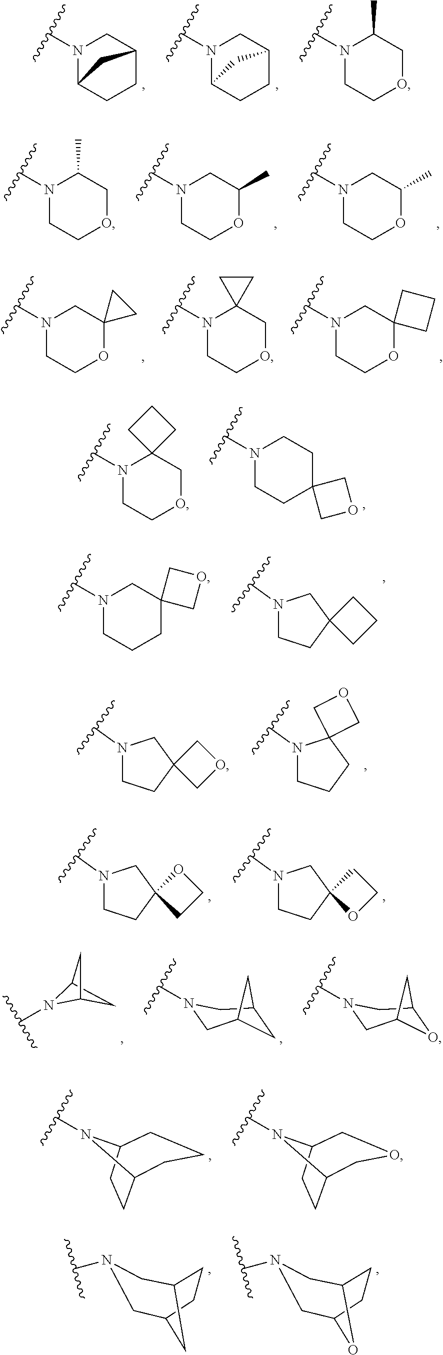

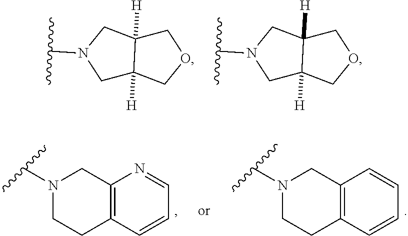

In another aspect, R₁ is

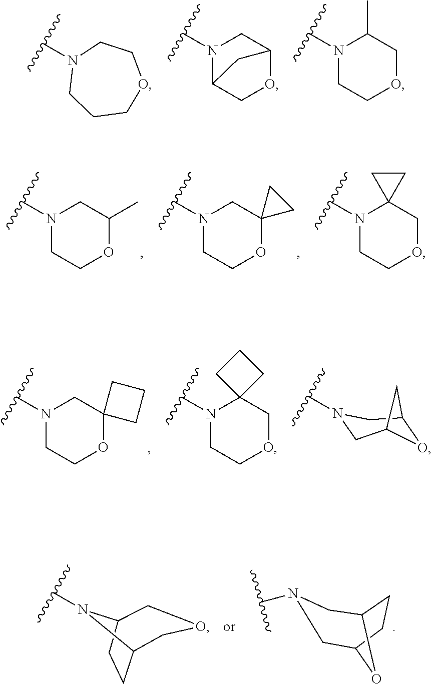

In another aspect, R₁ is

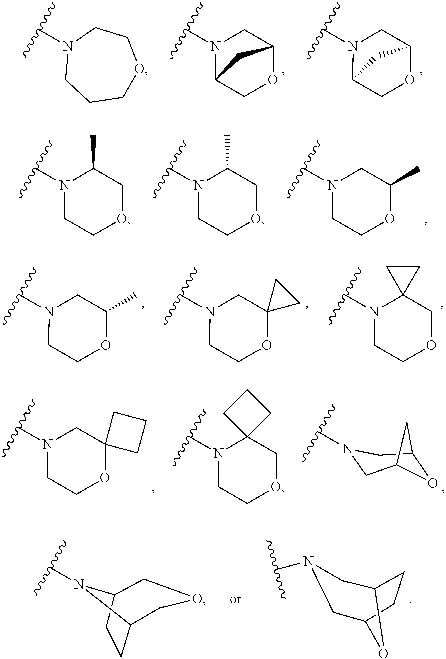

In another aspect, R₁ is

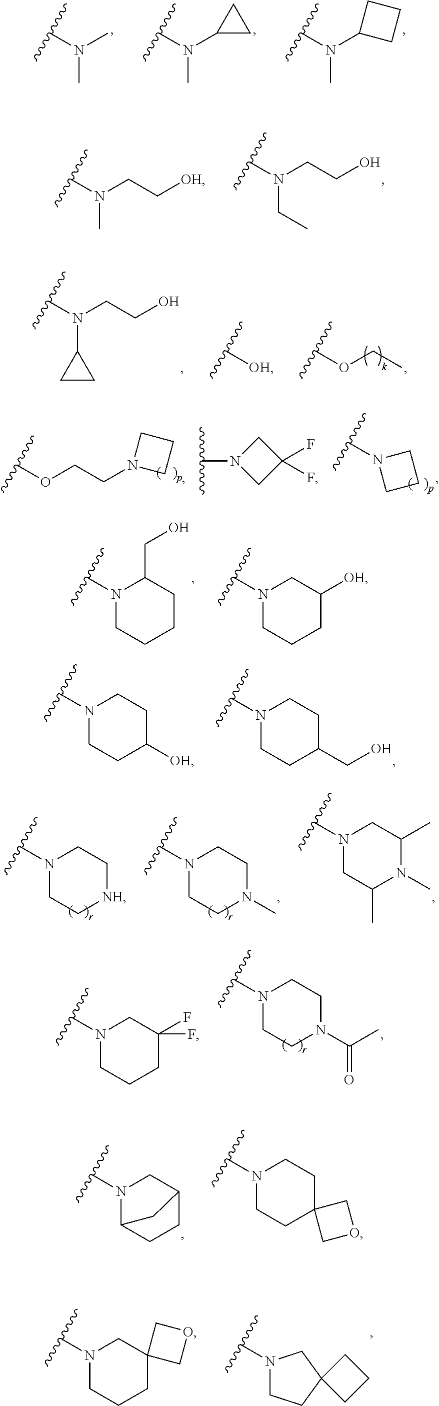

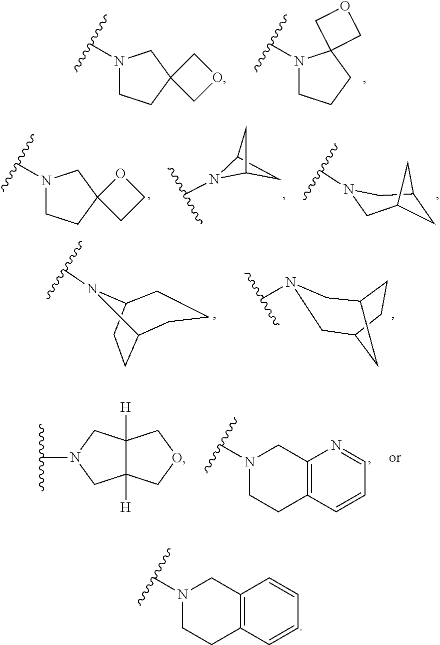

In another aspect, R₁ is

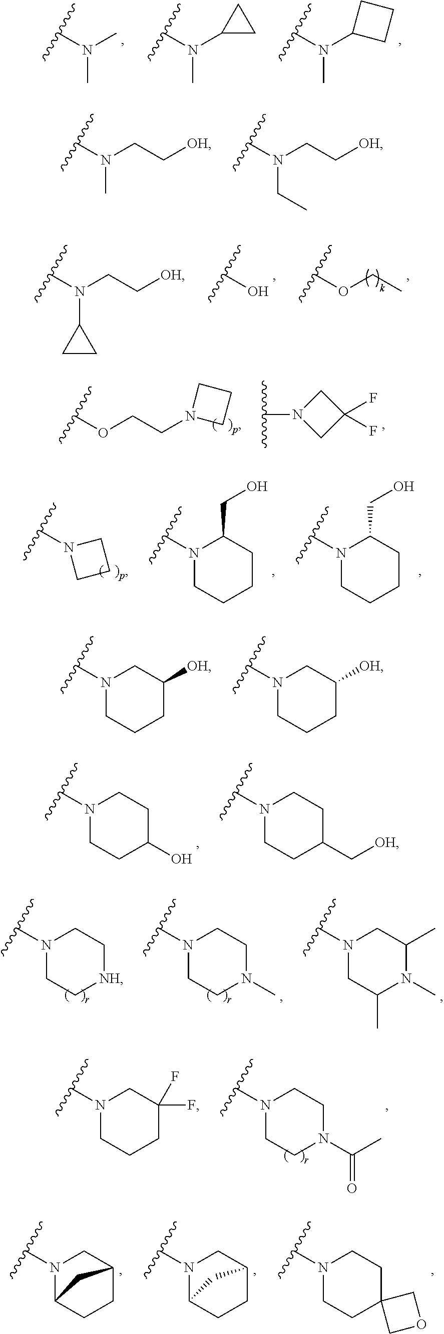

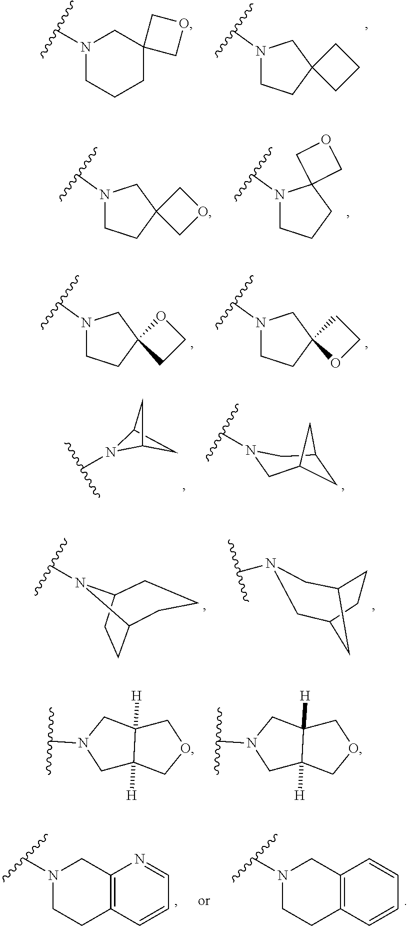

In another aspect, R₁ is

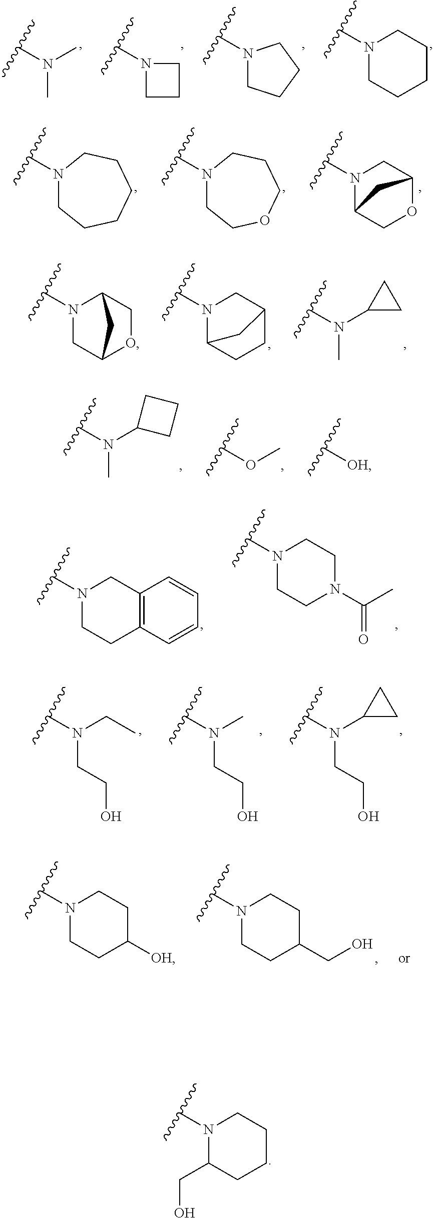

In another aspect R₁ is

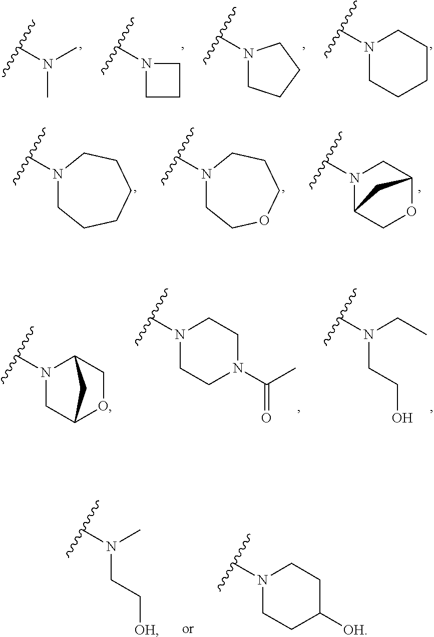

In one aspect, R₂ is NO₂, SO₂CF₃, or SO₂CF₂Cl. In one aspect, R₂ is NO₂ or SO₂CF₃. In one aspect, R₂ is NO₂ or SO₂CF₂Cl. In one aspect, R₂ is SO₂CF₃ or SO₂CF₂Cl. In one aspect, R₂ is NO₂. In another aspect, R₂ is SO₂CF₃. In one aspect, R₂ is SO₂CF₂Cl.

In another aspect, the compound of Formula (I), or a pharmaceutically acceptable salt, hydrate, solvate, or prodrug thereof, is of Formula (I-C):

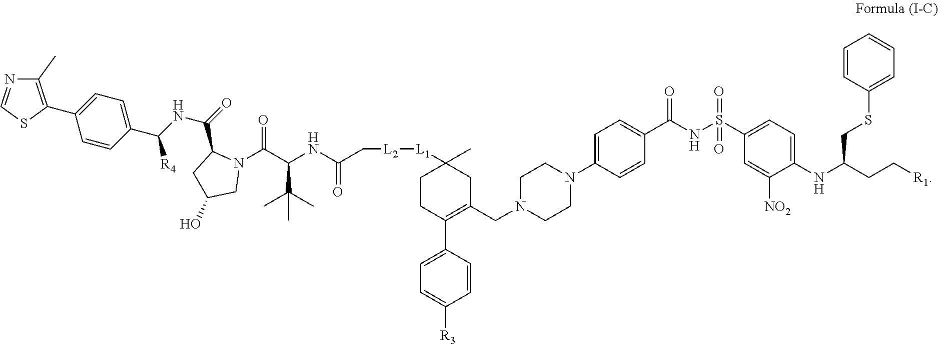

In another aspect, the compound of Formula (I), or a pharmaceutically acceptable salt, hydrate, solvate, or prodrug thereof, is of Formula (I-D):

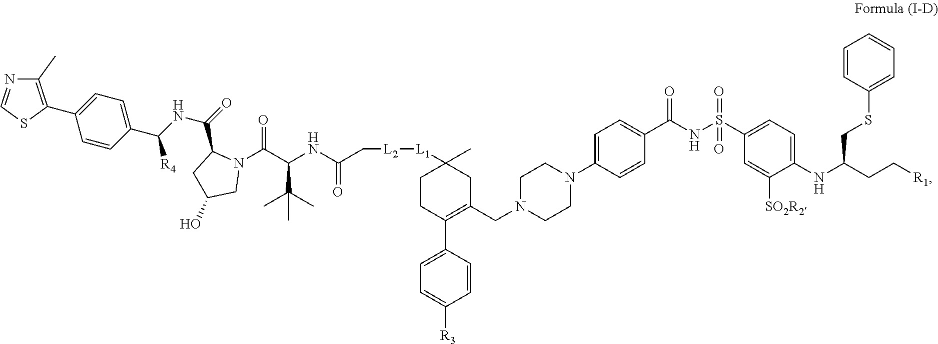

wherein R_{2′} is CF₃, or CF₂Cl.

In one aspect, R₃ is Cl, F, CFH₂, CF₂H, or CF₃. In some embodiments, R₃ is F, CFH₂, CF₂H, or CF₃. In certain embodiments, R₃ is CFH₂, CF₂H, or CF₃. In some embodiments, R₃ is Cl, F, or CF₃. In certain embodiments, R₃ is Cl or F. In some embodiments, R₃ is CF₃ or F. In certain embodiments, R₃ is Cl. In some embodiments, R₃ is F. In certain embodiments, R₃ is CF₃.

In some embodiments, R₃ is CF₂H. In certain embodiments, R₃ is CFH₂. In one aspect, R₄ is H or CH₃. In some embodiments, R₄ is H. In certain embodiments, R₄ is CH₃.

In one aspect, R₅ is independently H, optionally substituted alkyl, or optionally substituted cycloalkyl. In some embodiments, R₅ is independently H or optionally substituted cycloalkyl. In certain embodiments, R₅ is independently H or optionally substituted alkyl. In some embodiments, R₅ is independently optionally substituted alkyl or optionally substituted cycloalkyl. In certain embodiments, R₅ is independently H, unsubstituted alkyl, or unsubstituted cycloalkyl. In some embodiments, R₅ is independently H or unsubstituted cycloalkyl. In certain embodiments, R₅ is independently H or unsubstituted alkyl. In some embodiments, R₅ is independently unsubstituted alkyl or unsubstituted cycloalkyl.

In certain embodiments, R₅ is independently H, optionally substituted C₁-C₁₀ alkyl, or optionally substituted C₃-C₁₀ cycloalkyl. In some embodiments, R₅ is independently H or optionally substituted C₃-C₁₀ cycloalkyl. In certain embodiments, R₅ is independently H or optionally substituted C₁-C₁₀ alkyl. In some embodiments, R₅ is independently optionally substituted C₁-C₁₀ alkyl or optionally substituted C₃-C₁ cycloalkyl. In certain embodiments, R₅ is independently H, unsubstituted C₁-C₁₀ alkyl, or unsubstituted C₃-C₁ cycloalkyl. In some embodiments, R₅ is independently H or unsubstituted C₃-C₁₀ cycloalkyl. In certain embodiments, R₅ is independently H or unsubstituted C₁-C₁₀ alkyl. In some embodiments, R₅ is independently unsubstituted C₁-C₁₀ alkyl or unsubstituted C₃-C₁₀ cycloalkyl. In certain embodiments, R₅ is independently H, optionally substituted C₁-C₆ alkyl, or optionally substituted C₃-C₇ cycloalkyl. In some embodiments, R₅ is independently H or optionally substituted C₃-C₇ cycloalkyl. In certain embodiments, R₅ is independently H or optionally substituted C₁-C₆ alkyl. In some embodiments, R₅ is independently optionally substituted C₁-C₆ alkyl or optionally substituted C₃-C₇ cycloalkyl. In certain embodiments, R₅ is independently H, unsubstituted C₁-C₆ alkyl, or unsubstituted C₃-C₇ cycloalkyl. In some embodiments, R₅ is independently H or unsubstituted C₃-C₇ cycloalkyl. In certain embodiments, R₅ is independently H or unsubstituted C₁-C₆ alkyl. In some embodiments, R₅ is independently unsubstituted C₁-C₆ alkyl or unsubstituted C₃-C₇ cycloalkyl. In certain embodiments, R₅ is H, methyl, ethyl, propyl, butyl, pentyl, hexyl, cyclopropyl, cyclobutyl, cyclopentyl, cyclohexyl, or cycloheptyl. In some embodiments, R₅ is H, CH₃, or CH₂CH₃. In some embodiments, R₅ is H or CH₃. In some embodiments, R₅ is CH₃ or CH₂CH₃.

In certain embodiments, R₅ is H. In some embodiments, R₅ is optionally substituted C₁-C₆ alkyl. In certain embodiments, R₅ is unsubstituted C₁-C₆ alkyl. In some embodiments, R₅ is CH₃. In certain embodiments, R₅ is CH₂CH₃. In some embodiments, R₅ is optionally substituted C₃-C₇ cycloalkyl. In certain embodiments, R₅ is unsubstituted C₃-C₇ cycloalkyl.

In some embodiments, L₁ is

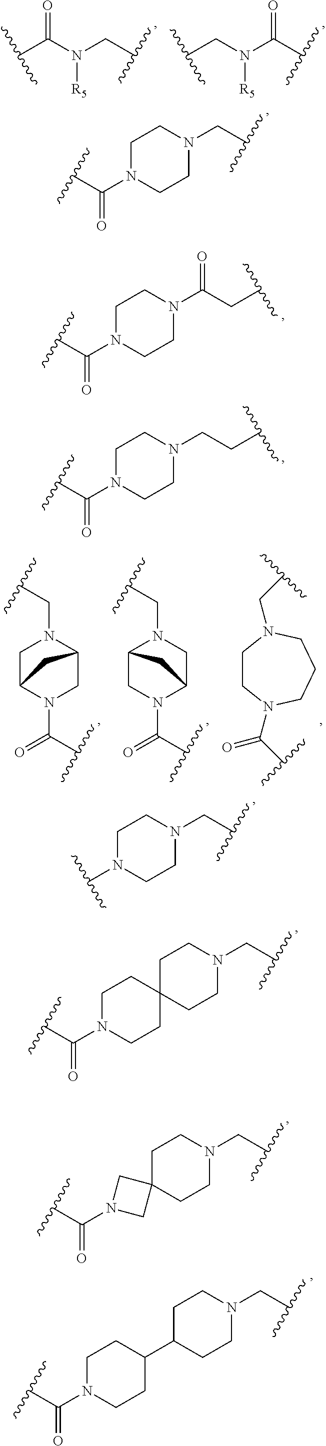

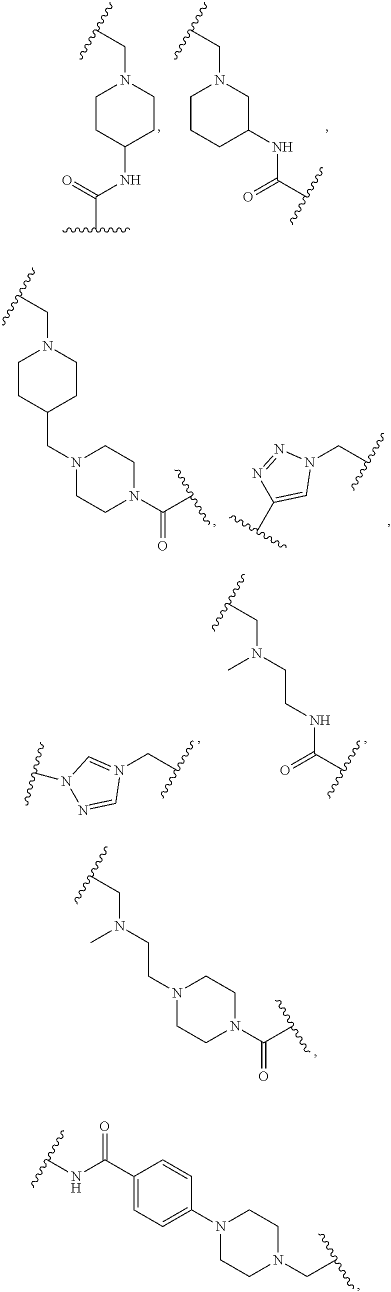

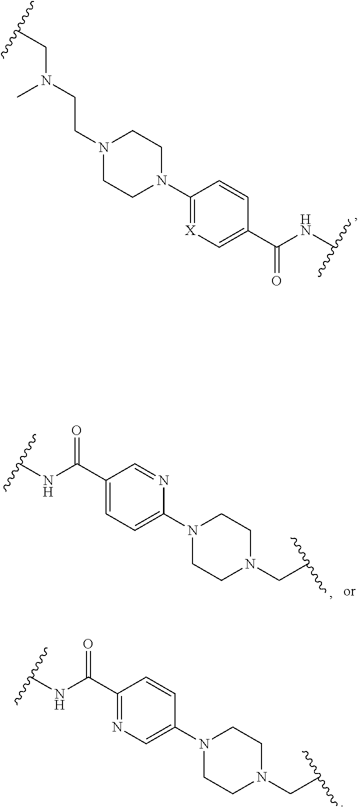

In some embodiments, L₁ is

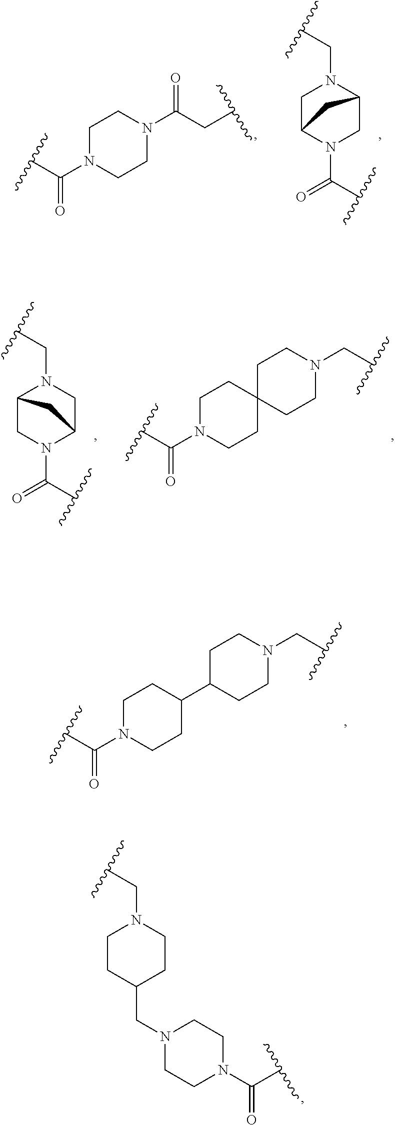 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2 ; *CN1CCC(C2CCN(C(*)=O)CC2)CC1 ; *CN1CCC(CN2CCN(C(*)=O)CC2)CC1]

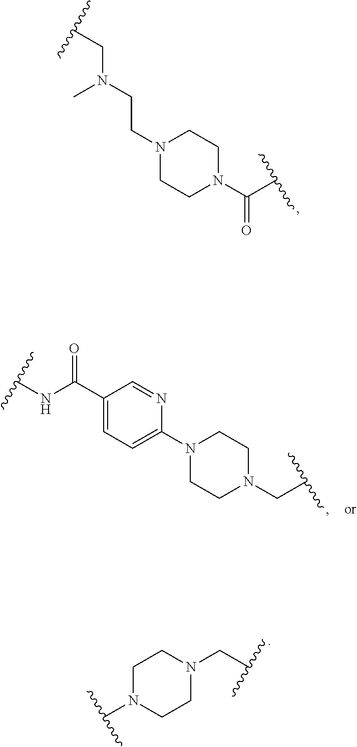 [SMILES: *CN(C)CCN1CCN(C(*)=O)CC1 ; *CN1CCN(c2ccc(C(=O)N*)cn2)CC1 ; *CN1CCN(*)CC1]

In some embodiments, L₁ is

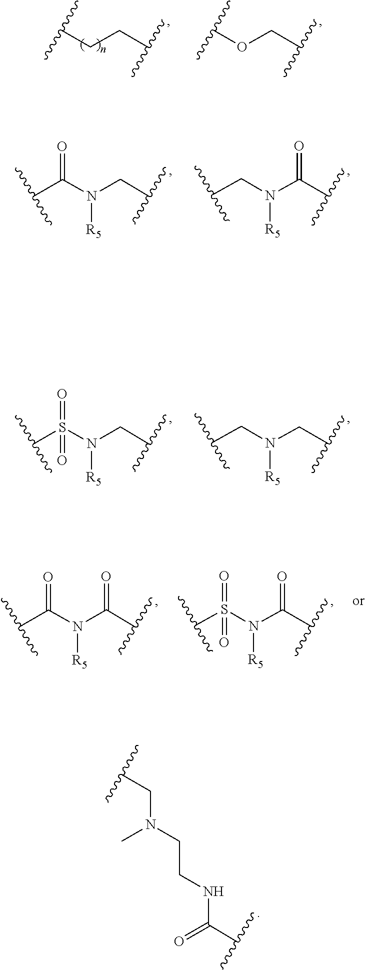

In some embodiments, L₁ is

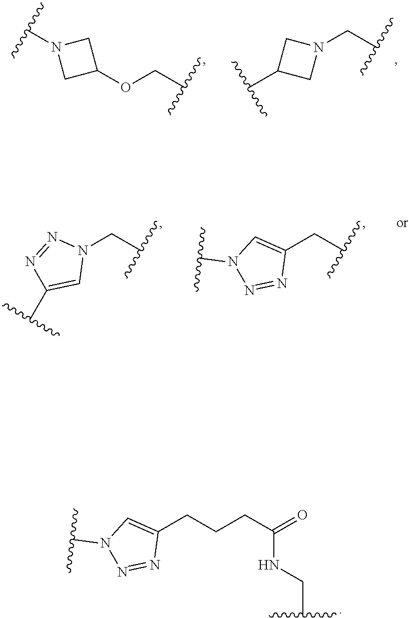 [SMILES: *COC1CN(*)C1 ; *CN1CC(*)C1 ; *Cn1cc(*)nn1 ; *Cc1cn(*)nn1 ; *CNC(=O)CCCc1cn(*)nn1]

In some embodiments, In some embodiments, L₁ is

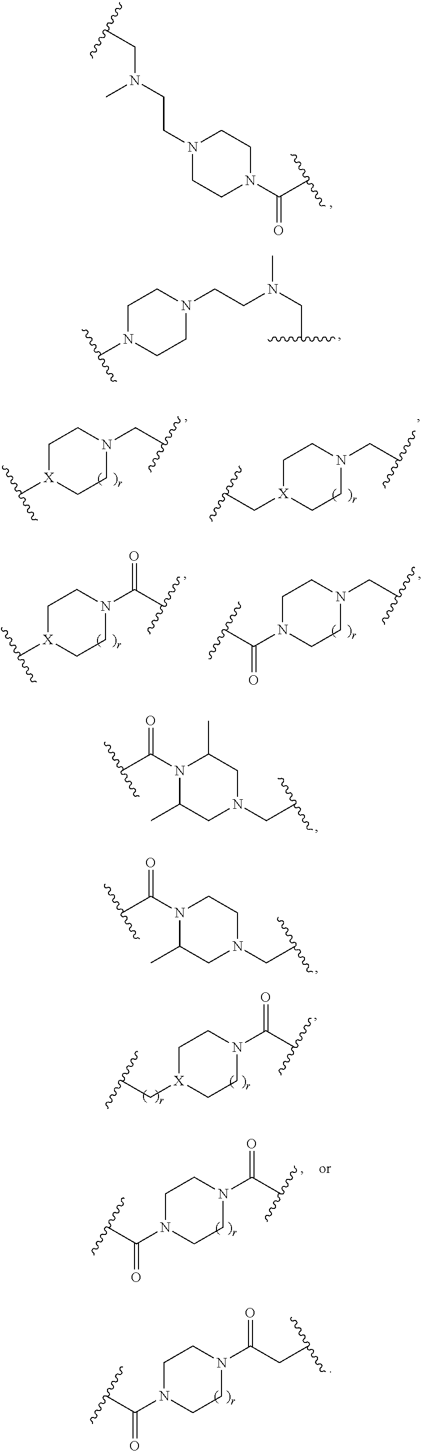

In some embodiments, L₁ is

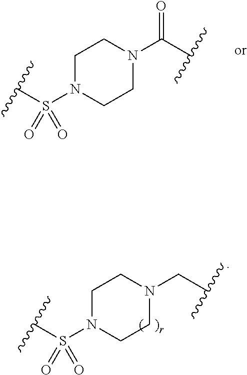

In some embodiments, L₁ is

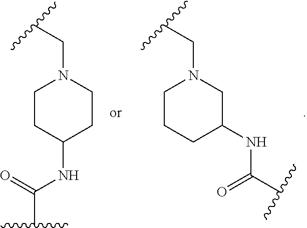 [SMILES: *CN1CCC(NC(*)=O)CC1 ; *CN1CCCC(NC(*)=O)C1]

In some embodiments, L₁ is

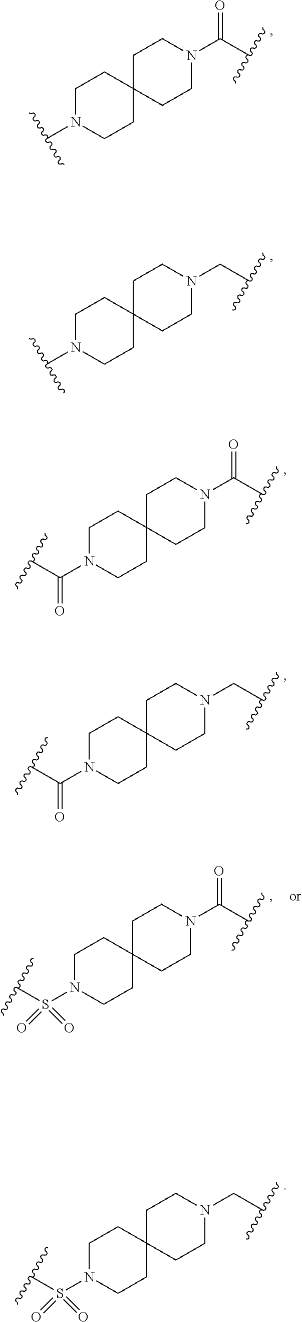 [SMILES: *C(=O)N1CCC2(CCN(*)CC2)CC1 ; *CN1CCC2(CCN(*)CC2)CC1 ; *C(=O)N1CCC2(CC1)CCN(C(*)=O)CC2 ; *CN1CCC2(CC1)CCN(C(*)=O)CC2 ; *C(=O)N1CCC2(CC1)CCN(S(*)(=O)=O)CC2 ; *CN1CCC2(CC1)CCN(S(*)(=O)=O)CC2]

In some embodiments, L₁ is

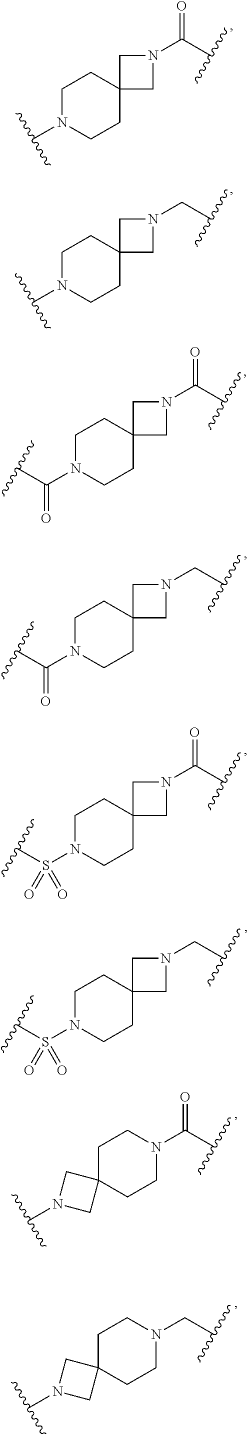

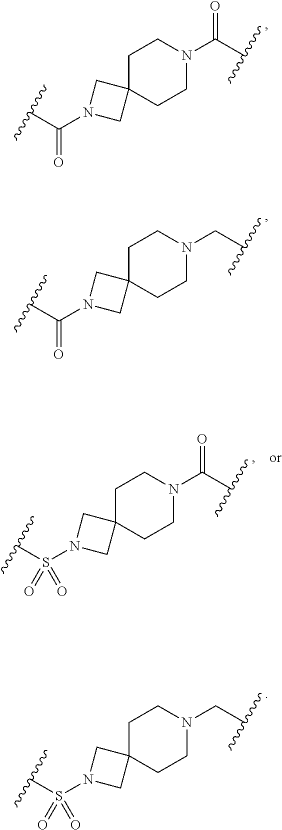 [SMILES: *C(=O)N1CCC2(CC1)CN(C(*)=O)C2 ; *CN1CCC2(CC1)CN(C(*)=O)C2 ; *C(=O)N1CCC2(CC1)CN(S(*)(=O)=O)C2 ; *CN1CCC2(CC1)CN(S(*)(=O)=O)C2]

In some embodiments L₁ is

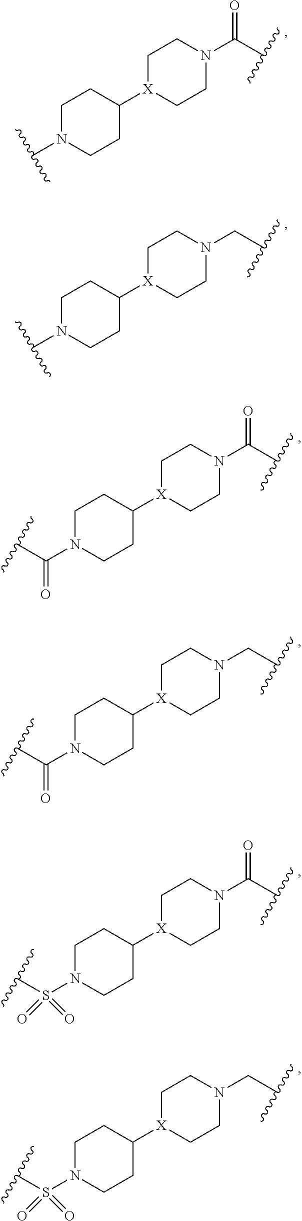

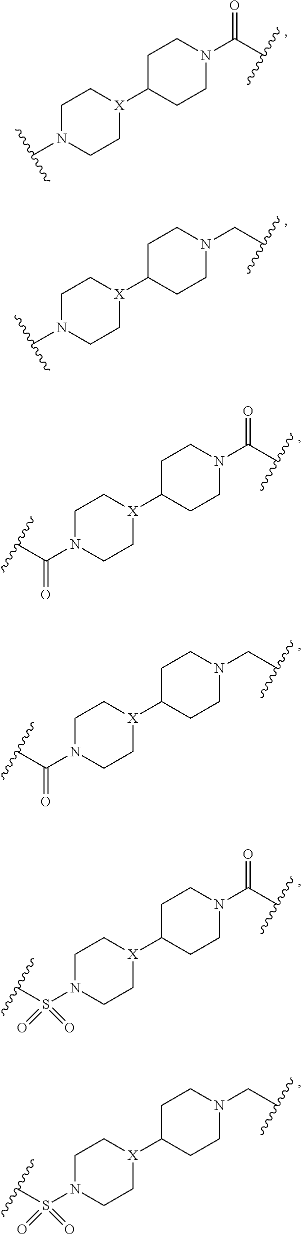

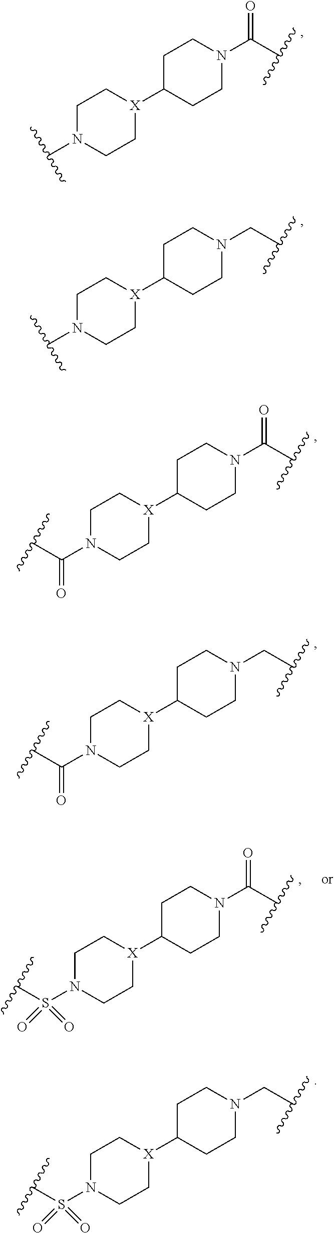

In some embodiments, L₁ is

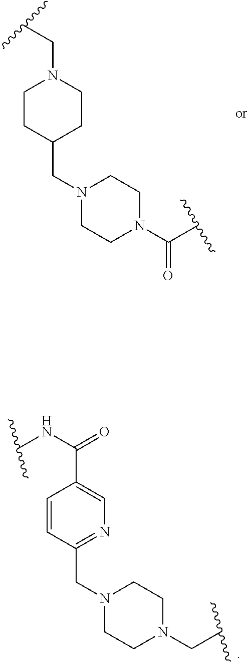 [SMILES: *CN1CCC(CN2CCN(C(*)=O)CC2)CC1 ; *CN1CCN(Cc2ccc(C(=O)N*)cn2)CC1]

In certain embodiments, L₁ is

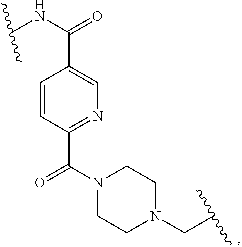 [SMILES: *CN1CCN(C(=O)c2ccc(C(=O)N*)cn2)CC1]

In some embodiments, L₁ is

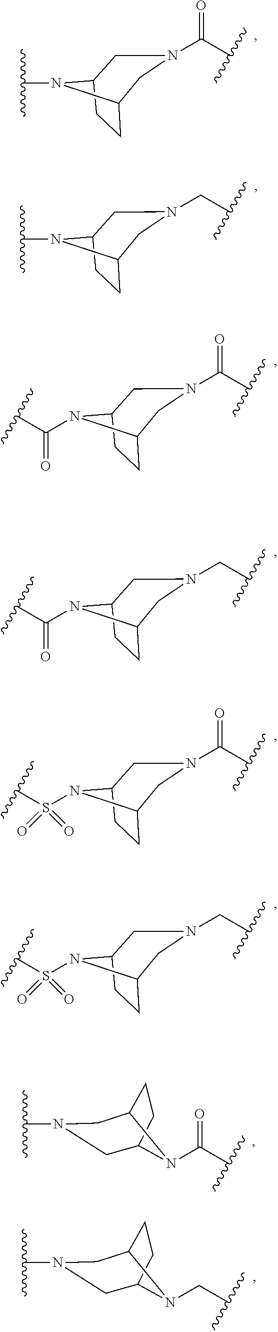

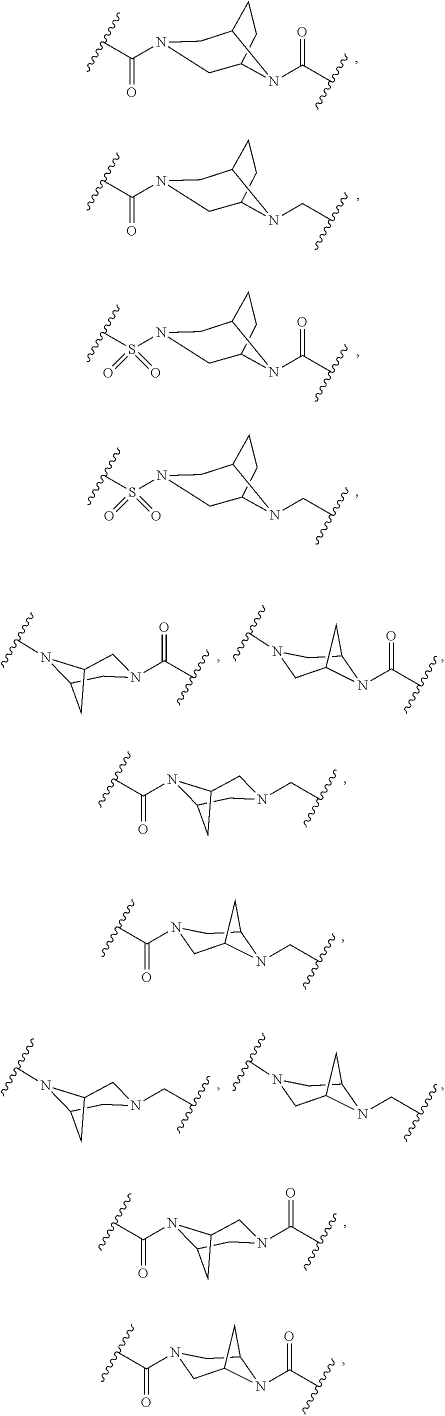

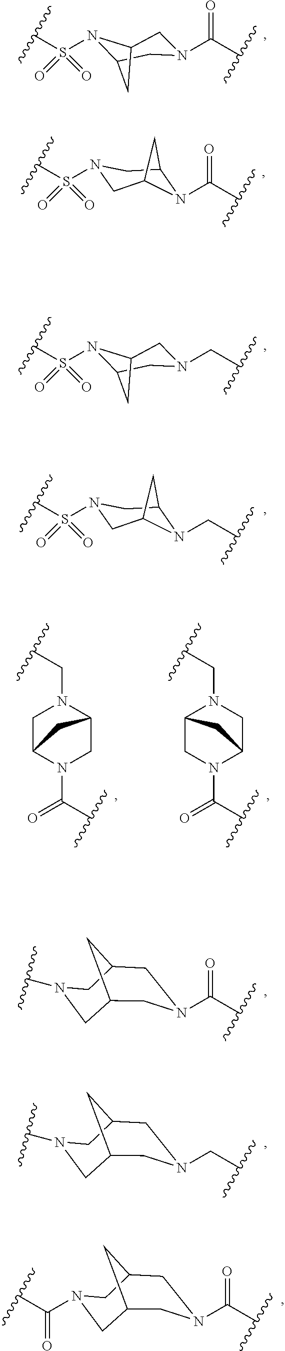

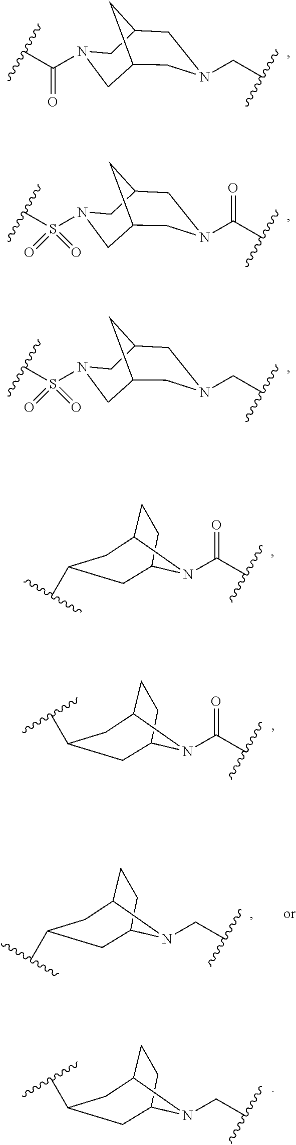

In some embodiments, L₁ is

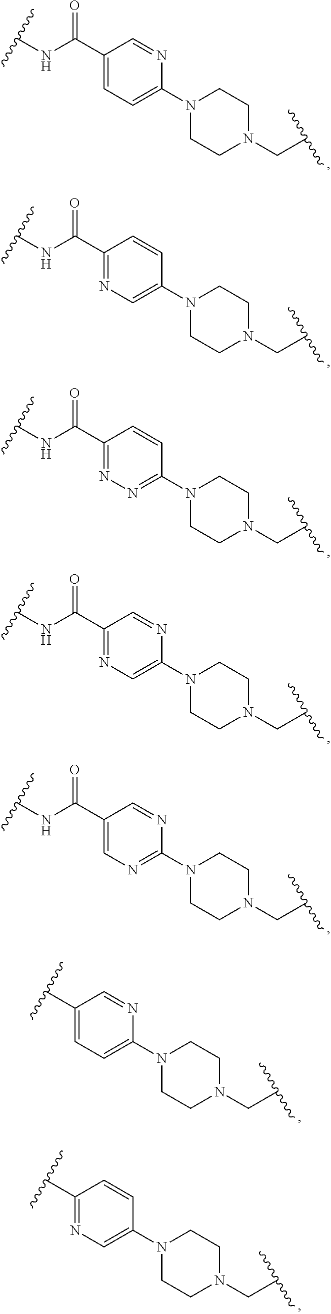

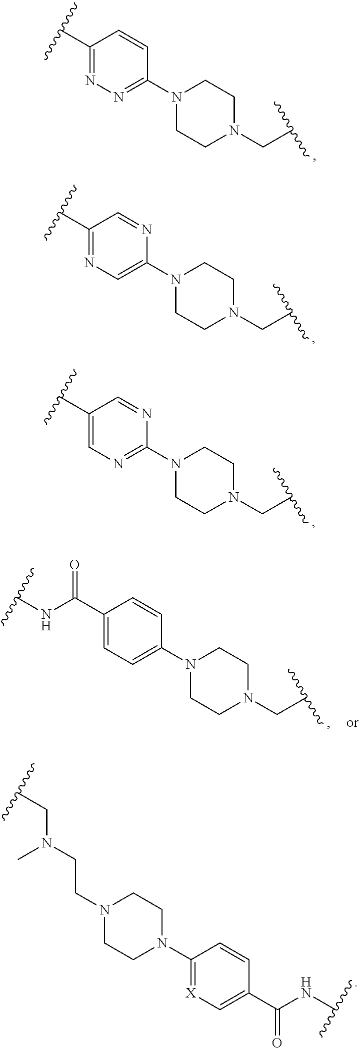

In one aspect, L₁ is

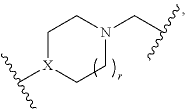

### X is N, and r is 1.

In some embodiments, L₂ is

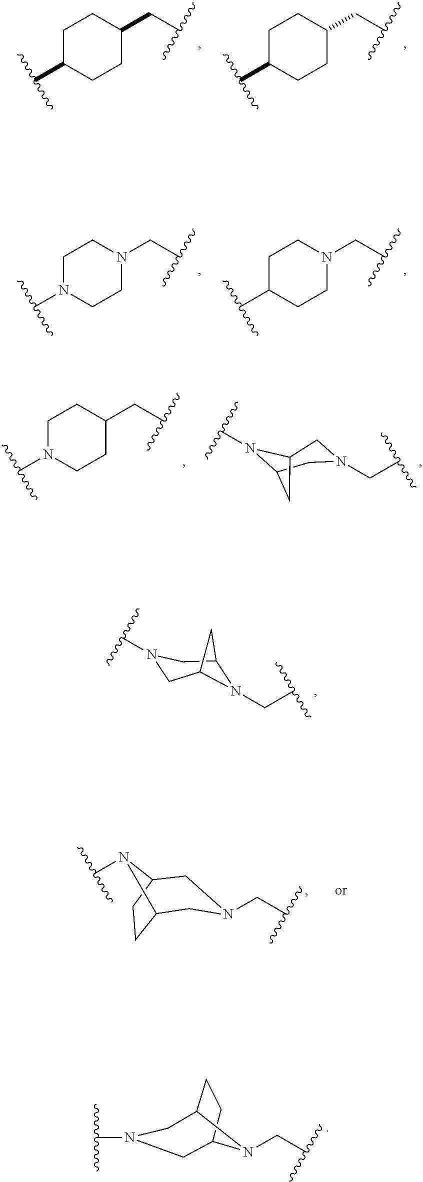

In some embodiments, L₂ is

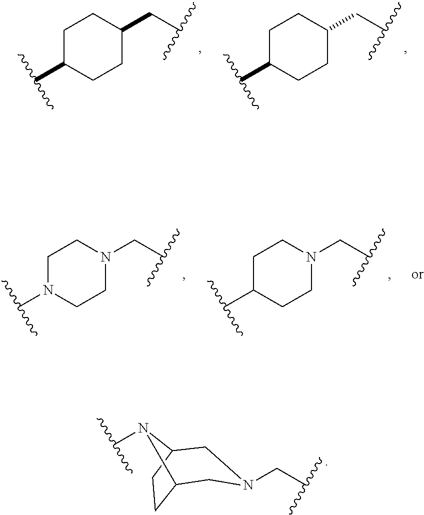 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

In some embodiments, L₁ is

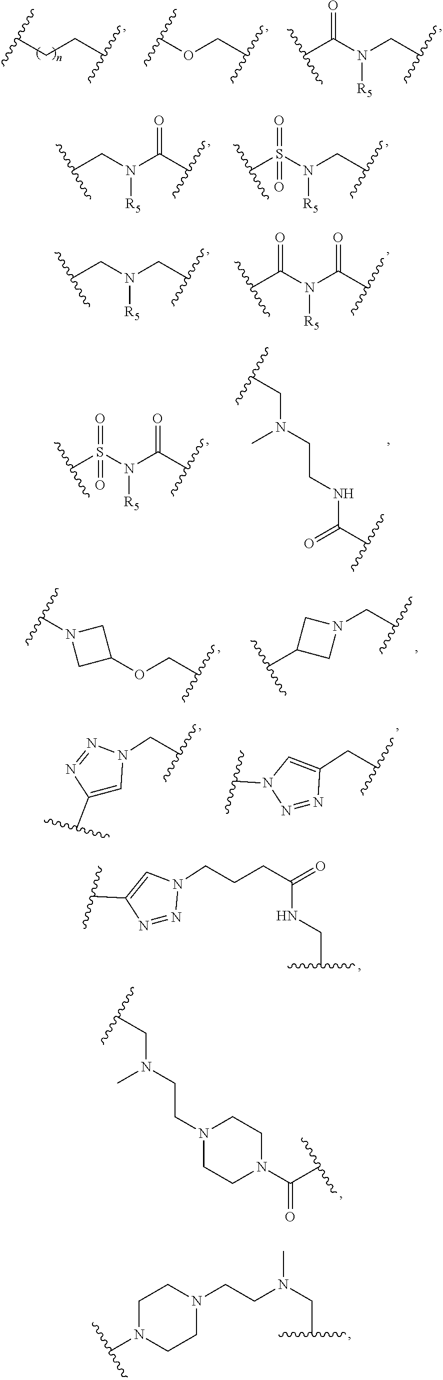

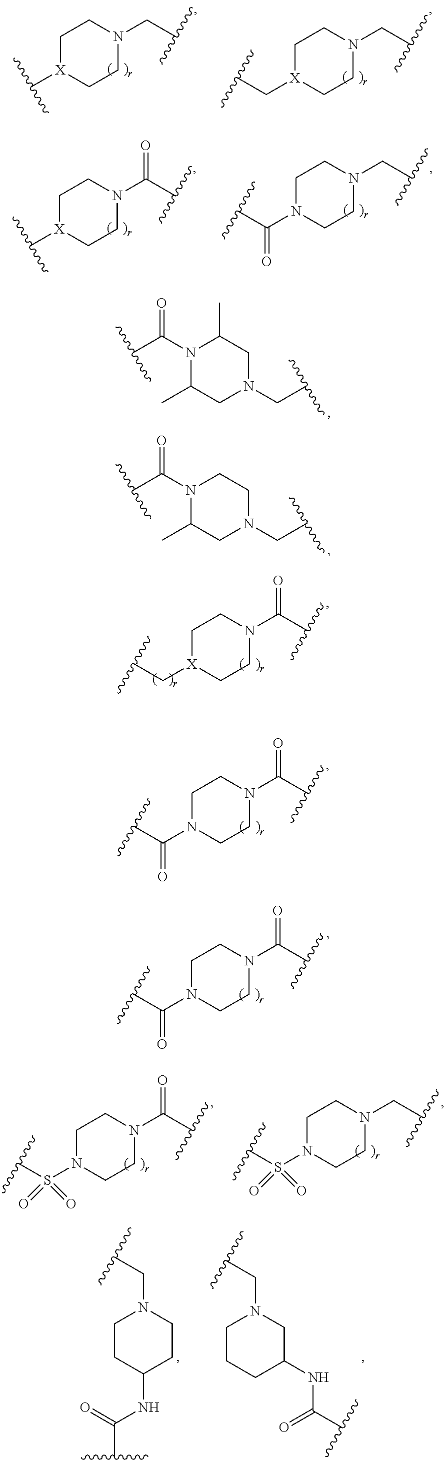

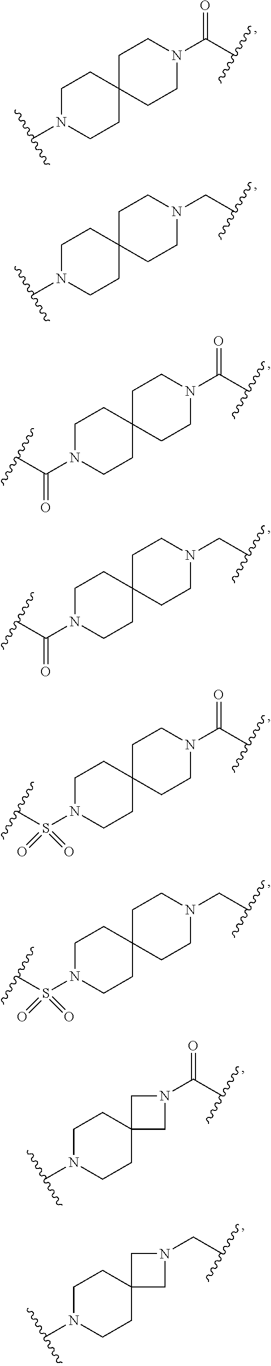

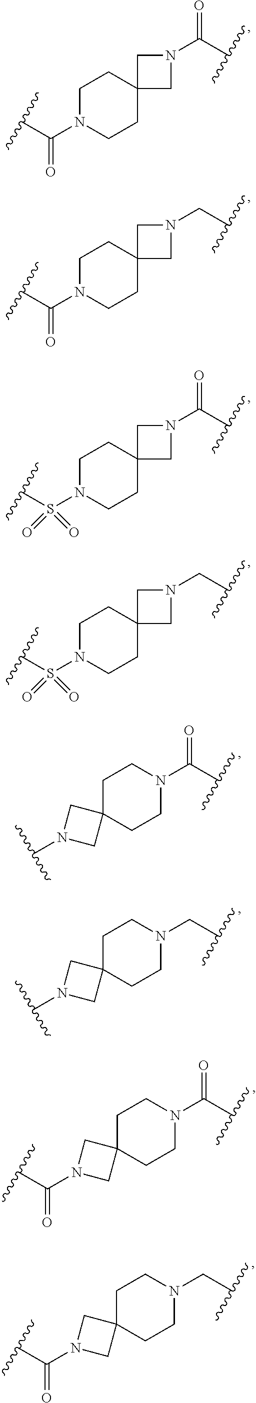

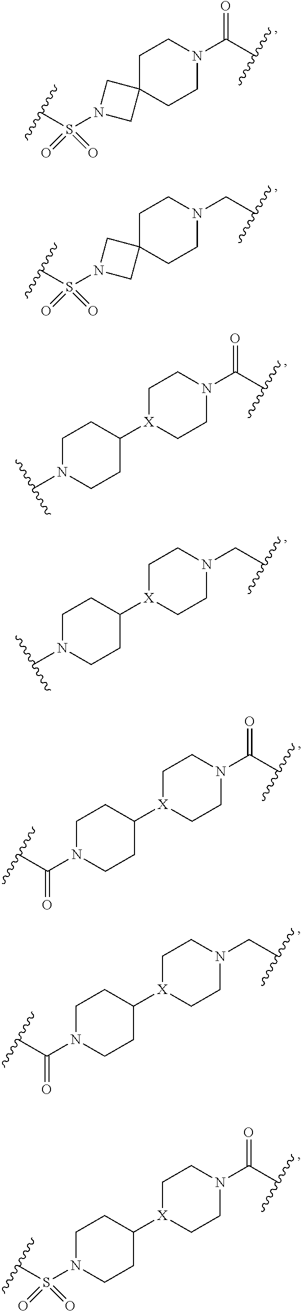

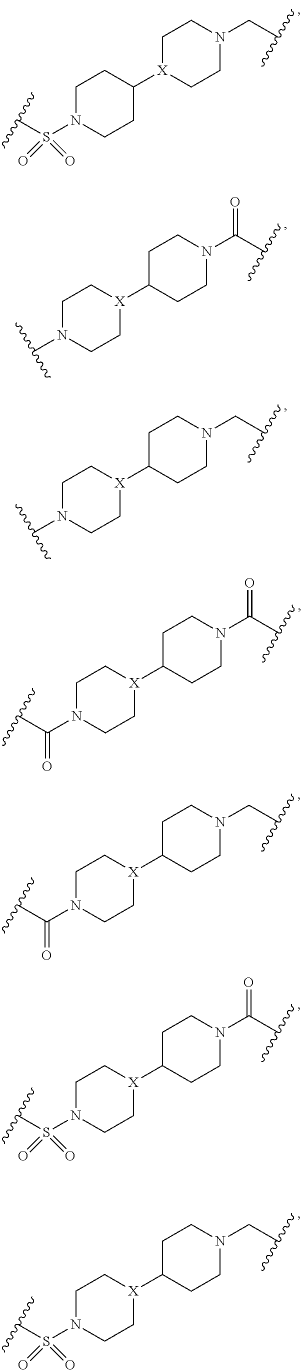

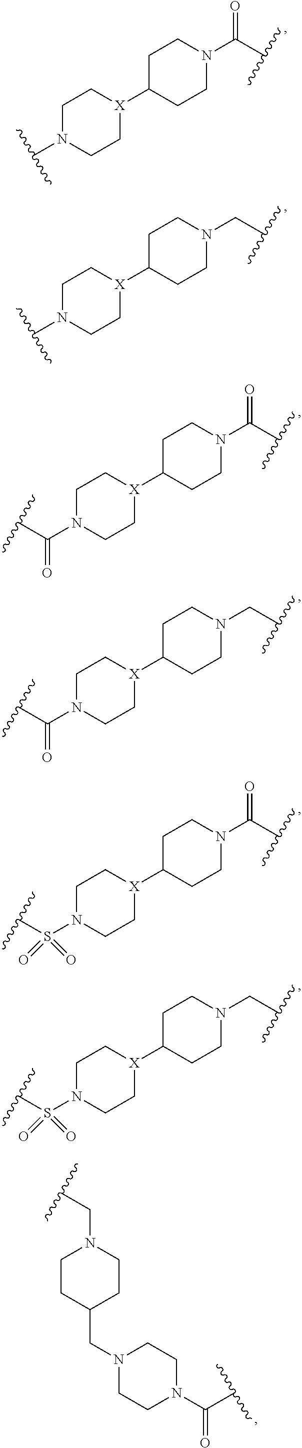

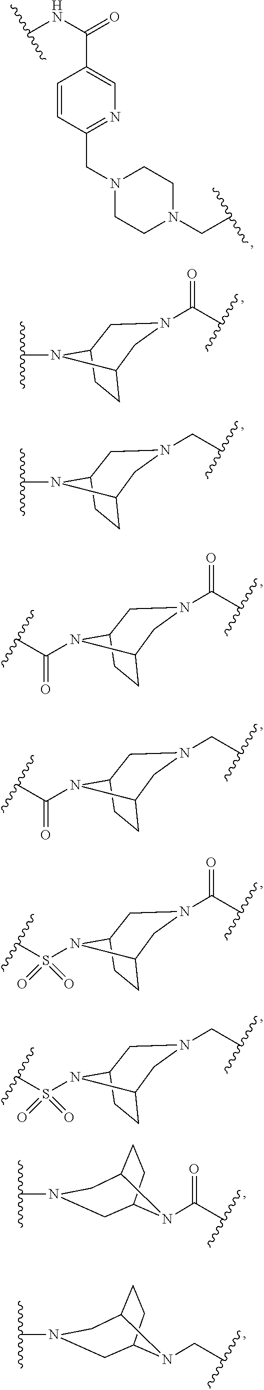

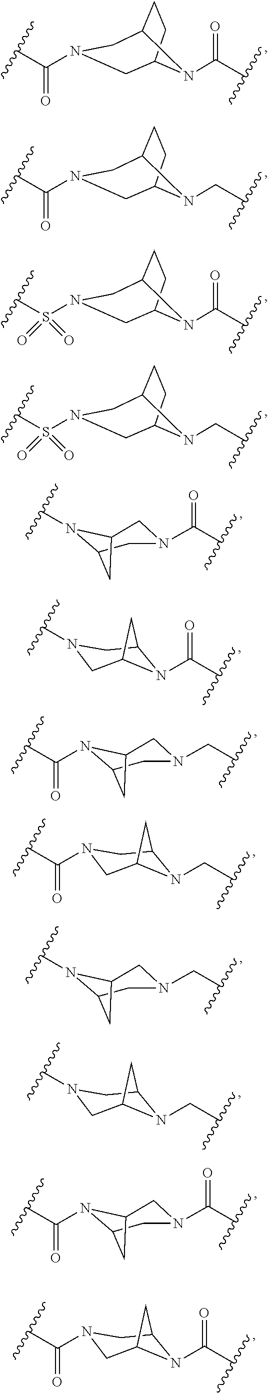

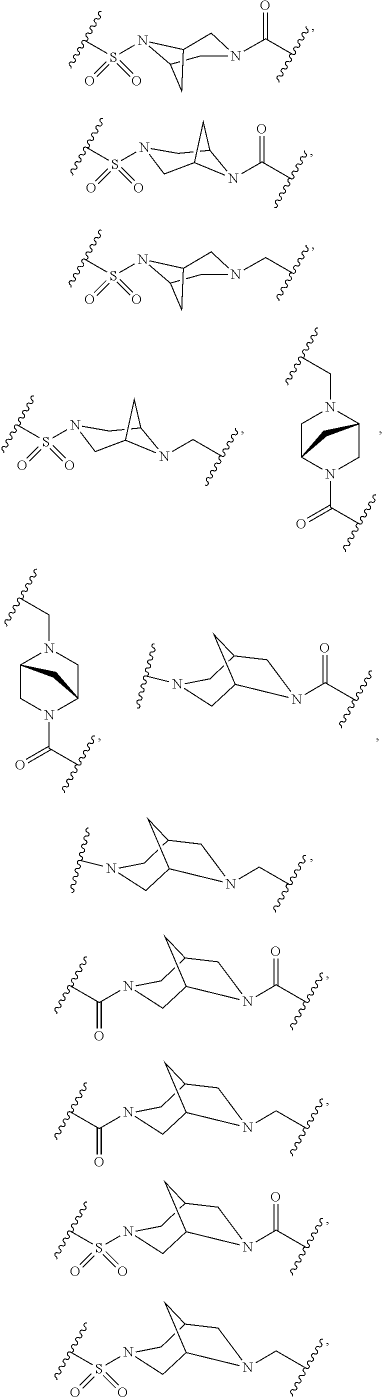

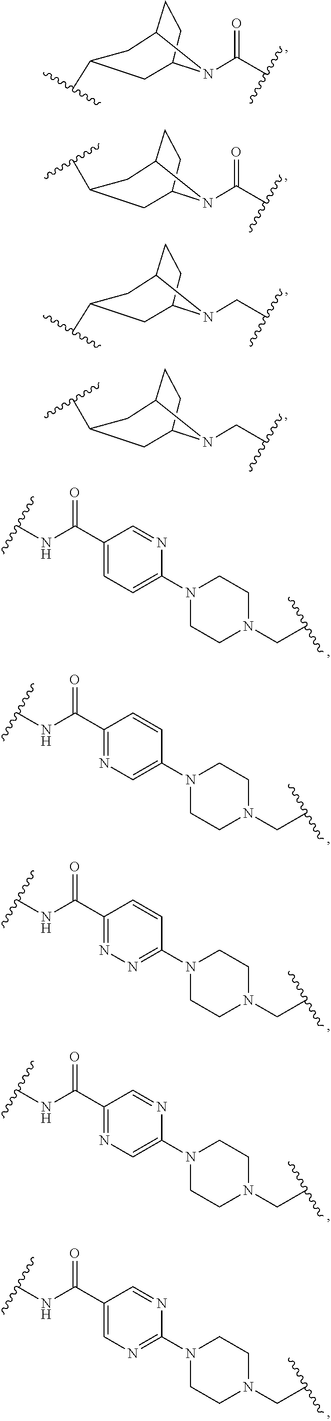

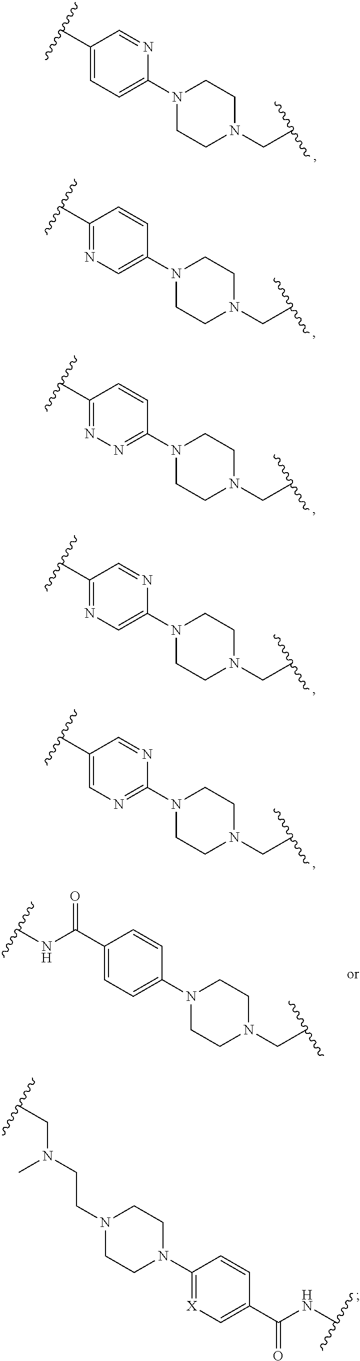

and
L₂ is a bond,

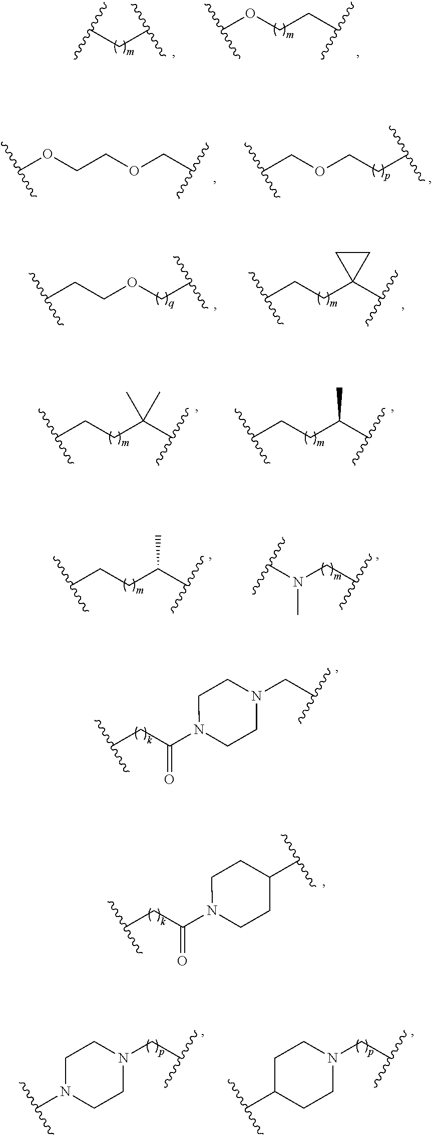

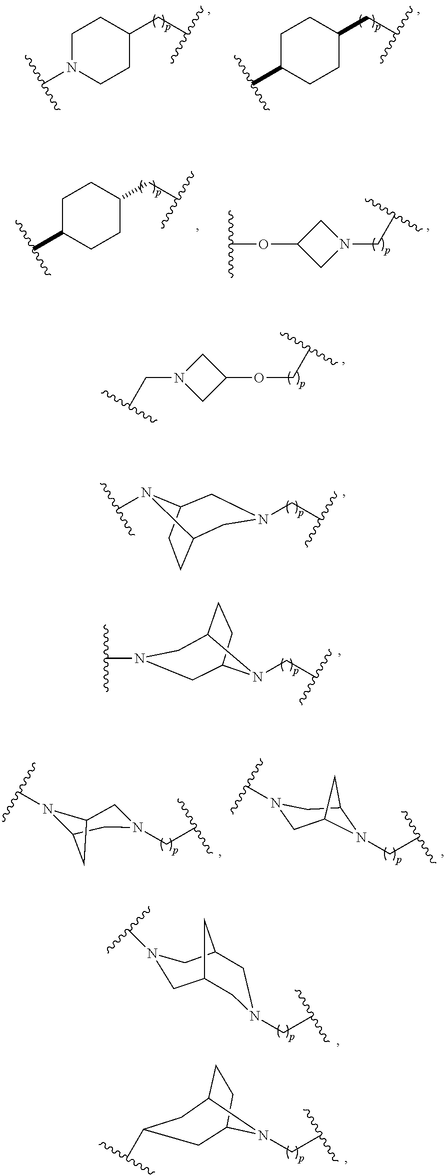

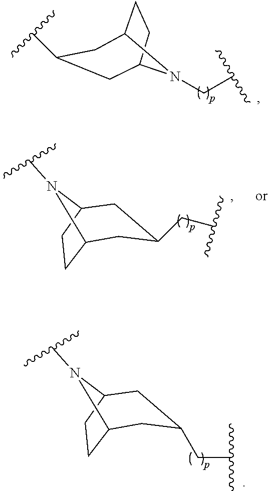

In some embodiments, L₂ is a bond,

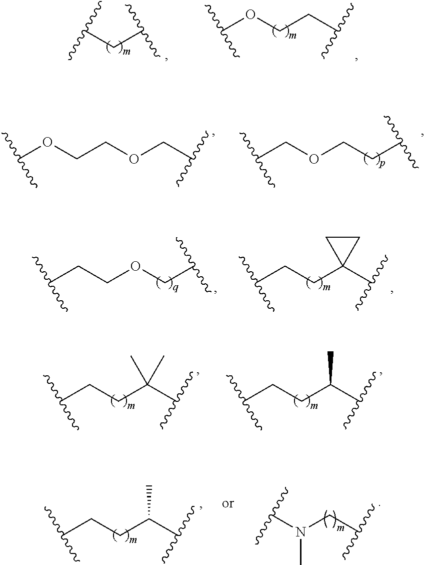

In some embodiments, L₂ is

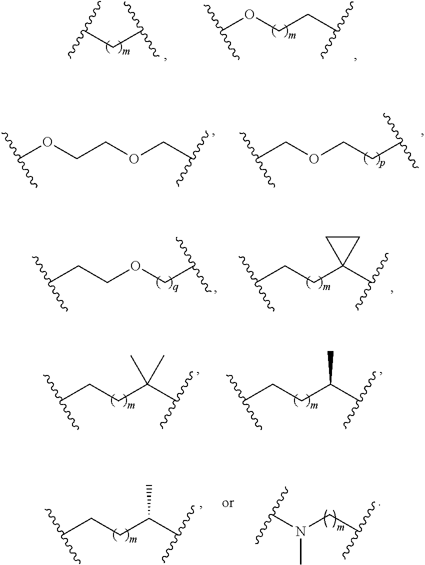

In some embodiments, L₂ is

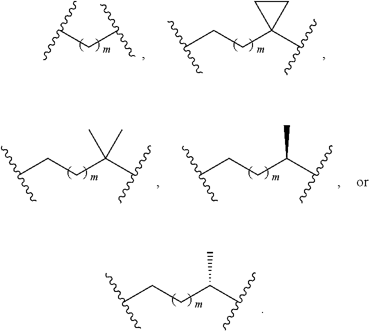

In some embodiments, L₂ is

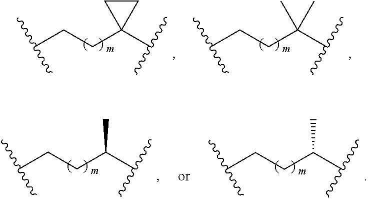

In some embodiments, L₂ is

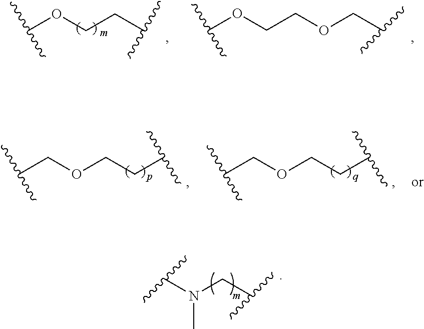

In some embodiments, L₂ is

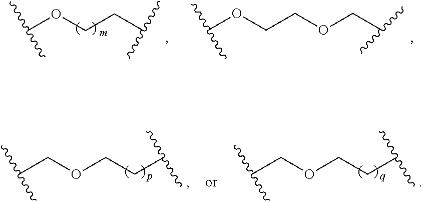

In some embodiments, L₁ is

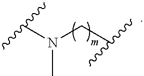

In some embodiments L₂ is a bond. In some embodiments, L₂ is

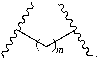

In some embodiments, L₂ is

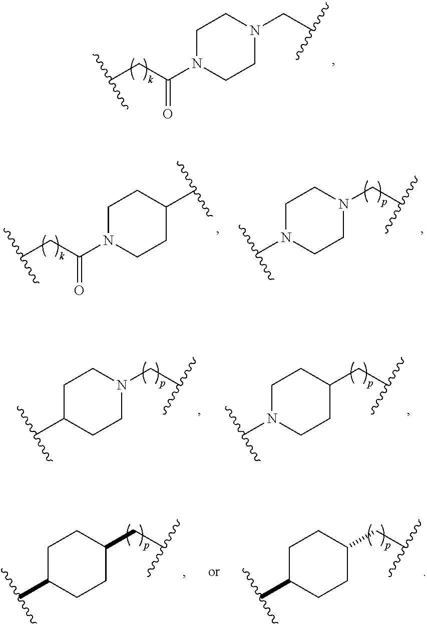

In some embodiments, L₂ is

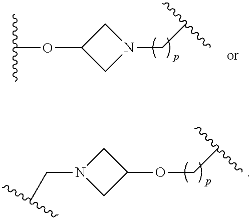

In some embodiments, L₂ is

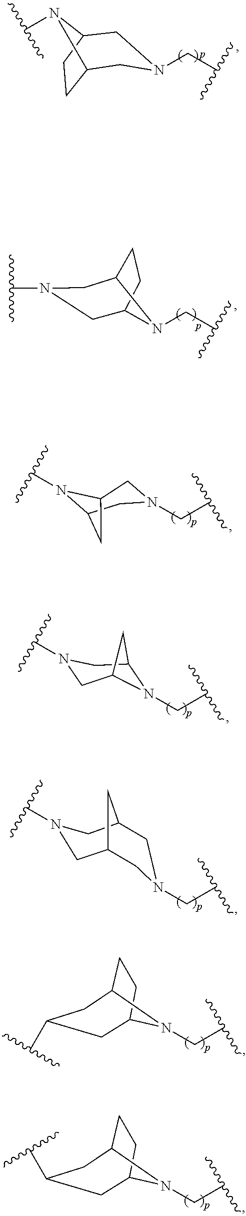

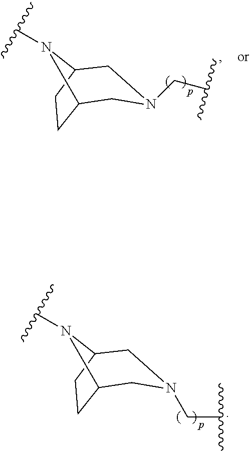

In some embodiments, L₂ is

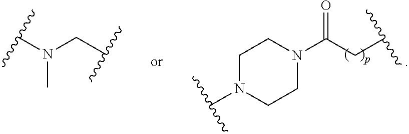

As defined herein, X is independently CH or N. In some embodiments, X is CH. In certain embodiments, X is N.

As defined herein, each k is independently 0, 1, 2, 3, 4, 5, or 6. In certain embodiments, each k is independently 0, 1, 2, 3, 4, or 5. In some embodiments, each k is independently 0, 1, 2, 3, or 4. In certain embodiments, each k is independently 0, 1, 2, or 3. In some embodiments, each k is independently 0, 1, or 2. In certain embodiments, each k is independently 3, 4, 5, or 6. In some embodiments, each k is independently 1, 2, 3, 4, 5, or 6. In certain embodiments, k is 0. In some embodiments, k is 1. In certain embodiments, k is 2. In some embodiments, k is 3. In certain embodiments, k is 4. In some embodiments, k is 5. In some embodiments, k is 6.

In one aspect, each m is independently 2, 3, 4, 5, 6, or 7. In some embodiments, each m is independently 2, 3, 4, 5, or 6. In certain embodiments, each m is independently 3, 4, 5, 6, or 7. In some embodiments, each m is independently 2, 3, 4, or 5. In certain embodiments, each m is independently 3, 4, 5, or 6. In some embodiments, each m is independently 4, 5, 6, or 7. In certain embodiments, each m is independently 2, 3, or 4. In some embodiments, each m is independently 3, 4, or 5. In certain embodiments, each m is independently 4, 5, or 6. In some embodiments, each m is 5, 6, or 7. In certain embodiments, each m is independently 2 or 3. In some embodiments, each m is independently 3 or 4. In certain embodiments, each m is independently 4 or 5. In some embodiments, each m is independently 5 or 6. In certain embodiments, each m is independently 6 or 7. In some embodiments, m is 1. In certain embodiments, m is 2. In some embodiments, m is 3. In certain embodiments, m is 4. In some embodiments, m is 5. In certain embodiments, m is 6. In some embodiments, m is 7.

In one aspect, each n is independently 0, 1, 2, 3, 4, 5, 6, 7, 8, 9, or 10. In some embodiments, each n is independently 0, 1, 2, 3, 4, 5, 6, 7, 8, or 9. In certain embodiments, each n is independently 1, 2, 3, 4, 5, 6, 7, 8, 9, or 10. In some embodiments, each n is independently 0, 1, 2, 3, 4, 5, 6, 7, or 8. In some embodiments, each n is independently 1, 2, 3, 4, 5, 6, 7, 8, or 9. In certain embodiments, each n is independently 2, 3, 4, 5, 6, 7, 8, 9, or 10. In some embodiments, each n is independently 0, 1, 2, 3, 4, or 5. In some embodiments, each n is independently 1, 2, 3, 4, 5, or 6. In certain embodiments, each n is independently 2, 3, 4, 5, 6, or 7. In some embodiments, each n is independently 3, 4, 5, 6, 7, or 8. In certain embodiments, each n is independently 4, 5, 6, 7, 8, or 9. In some embodiments, each n is independently 5, 6, 7, 8, 9, or 10. In certain embodiments, each n is independently 0, 1, 2, 3, or 4. In some embodiments, each n is independently 1, 2, 3, 4, or 5. In some embodiments, each n is independently 2, 3, 4, 5, or 6. In certain embodiments, each n is independently 3, 4, 5, 6, or 7. In some embodiments, each n is independently 0, 1, 2, or 3. In some embodiments, each n is independently 1, 2, 3, or 4. In some embodiments, each n is independently 2, 3, 4, or 5. In certain embodiments, each n is independently 3, 4, 5, or 6. In some embodiments, each n is independently 4, 5, 6, or 7. In certain embodiments, each n is independently 5, 6, 7, or 8. In some embodiments, each n is independently 6, 7, 8, or 9. In certain embodiments, each n is independently 7, 8, 9, or 10. In some embodiments, each n is independently 0, 1, or 2. In certain embodiments, each n is independently 1, 2, or 3. In certain embodiments, each n is independently 2, 3, or 4. In some embodiments, each n is independently 3, 4, or 5. In certain embodiments, each n is independently 4, 5, or 6. In some embodiments, each n is 5, 6, or 7. In certain embodiments, each n is independently 5, 6, or 7. In some embodiments, each n is 7, 8, or 9. In certain embodiments, each n is independently 8, 9, or 10. In some embodiments, n is 0 or 1. In some embodiments, n is 1 or 2. In certain embodiments, each n is independently 2 or 3. In some embodiments, each n is independently 3 or 4. In certain embodiments, each n is independently 4 or 5. In some embodiments, each n is independently 5 or 6. In certain embodiments, each n is independently 6 or 7. In some embodiments, each n is independently 7 or 8. In certain embodiments, each n is independently 8 or 9. In some embodiments, each n is independently 9 or 10. In certain embodiments, n is 0. In some embodiments, n is 1. In certain embodiments, n is 2. In some embodiments, n is 3. In certain embodiments, n is 4. In some embodiments, n is 5. In certain embodiments, n is 6. In some embodiments, n is 7. In certain embodiments, n is 8. In some embodiments, n is 9. In certain embodiments, n is 10.

In one aspect, each p is independently 0, 1, 2, 3, or 4. In some embodiments, each p Is independently 0, 1, 2, or 3. In certain embodiments, each p is independently 1, 2, 3, or 4. In some embodiments, each p is independently 0, 1, or 2. In certain embodiments, each p is independently 1, 2, or 3. In some embodiments, each p is independently 2, 3, or 4. In certain embodiments, each p is independently 0 or 1. In some embodiments, each p is independently 1 or 2. In certain embodiments, each p is independently 2 or 3. In some embodiments, each p is independently 3 or 4. In certain embodiments, p is 0. In some embodiments, p is 1. In certain embodiments, p is 2. In some embodiments, p is 3. In certain embodiments, p is 4.

In one aspect, each q is independently 1, 2, or 3. In certain embodiments, each q is independently 1 or 2. In some embodiments, each q is independently 2 or 3. In certain embodiments, q is 1. In some embodiments, q is 2. In certain embodiments, q is 3.

In one aspect, each r is independently 1 or 2. In some embodiments, r is 1. In certain embodiments, r is 2.

In one aspect, L₁ is

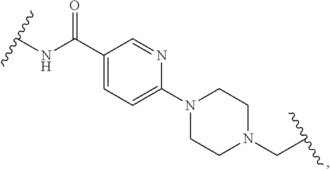 [SMILES: *CN1CCN(c2ccc(C(=O)N*)cn2)CC1]

and L₂ is bond or

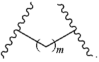

In another aspect, L₁ is

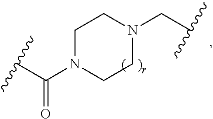

### and L₂ is

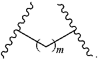

In another aspect, L₁ is

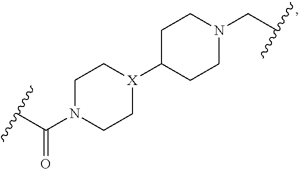

### and L₂ is

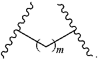

In another aspect, L₁ is

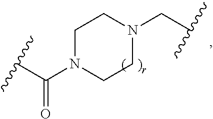

### and L₂ is

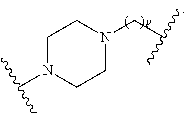

In some embodiments, L₂ is

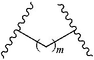

(e.g., wherein m is 1), and L₁ is

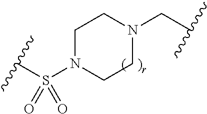

(e.g., wherein r is 1). In some embodiments, L₂ is

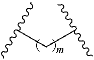

(e.g., wherein m is 3), and L₁ is

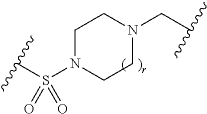

(e.g., wherein r is 1). In some embodiments, L₂ is

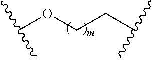

(e.g., wherein m is 2), and L₁ is

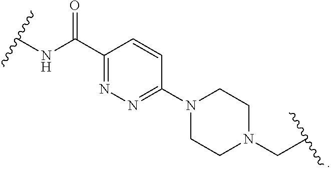 [SMILES: *CN1CCN(c2ccc(C(=O)N*)nn2)CC1]

In some embodiments, L₂ is

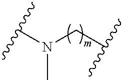

(e.g., wherein m is 3), and L₁ is

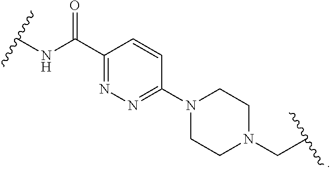 [SMILES: *CN1CCN(c2ccc(C(=O)N*)nn2)CC1]

In some embodiments, L₂ is

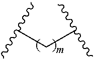

(e.g., wherein m is 4), and L₁ is

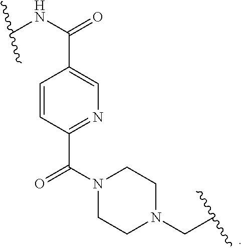 [SMILES: *CN1CCN(C(=O)c2ccc(C(=O)N*)cn2)CC1]

In some embodiments, L₂ is

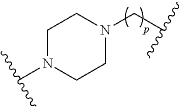

(e.g., wherein p is 2), and L₁ is

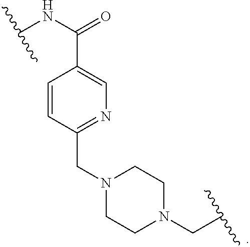 [SMILES: *CN1CCN(Cc2ccc(C(=O)N*)cn2)CC1]

In some embodiments, L₂ is

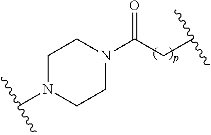

(e.g., wherein p is 1), and L₁ is

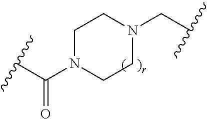

(e.g., wherein r is 1). In some embodiments, L₂ is

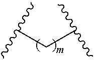

(e.g., wherein m is 3), and L₁ is

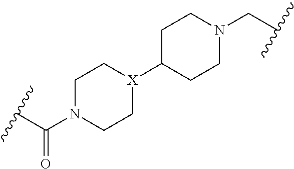

(e.g., wherein X is CH). In some embodiments, L₂ is

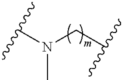

(e.g., wherein m is 2), and L₁ is

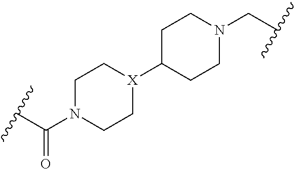

(e.g., wherein X is CH). In some embodiments, L₂ is

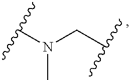 [SMILES: *CN(*)C]

### and L₁ is

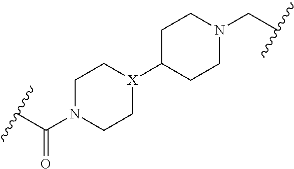

(e.g., wherein X is CH). In some embodiments, L₂ is

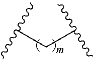

(e.g., wherein m is 3), and L₁ is

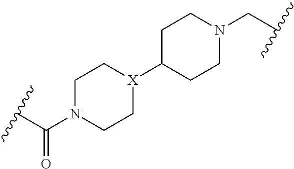

(e.g., wherein X is N). In some embodiments, L₂ is

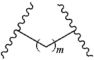

(e.g., wherein m is 3), and L₁ is

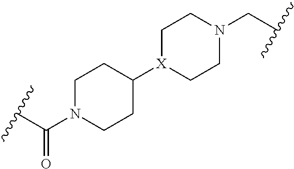

(e.g., wherein X is N). In another aspect, the compound of Formula (I) is:

| Com- | |
|---|---|
| pound | |
| # | Formula |
| 34 | 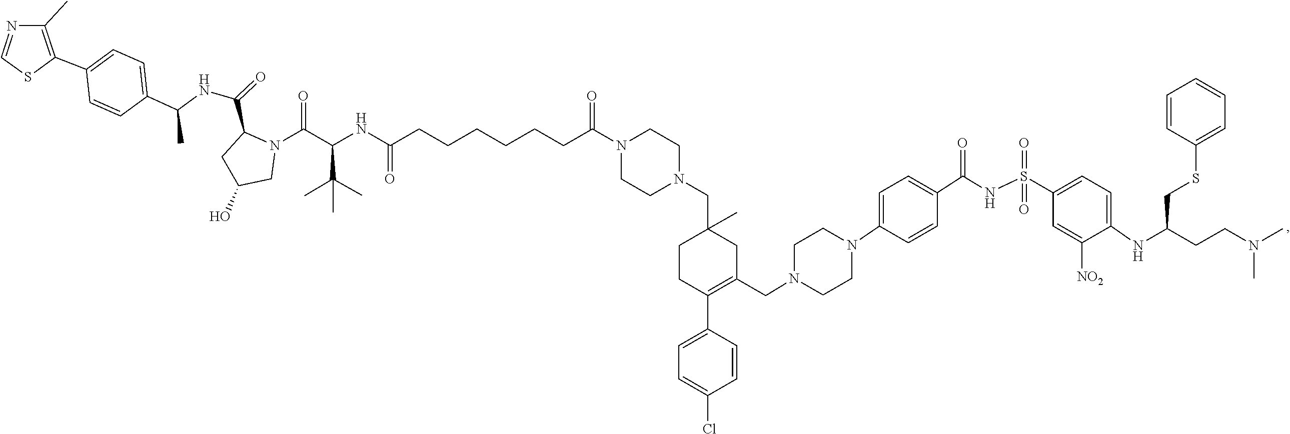 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c([N+](=O)[O-])c6)cc5)CC4)C3)CC2)C(C)(C)C)cc1] |
| 35 | 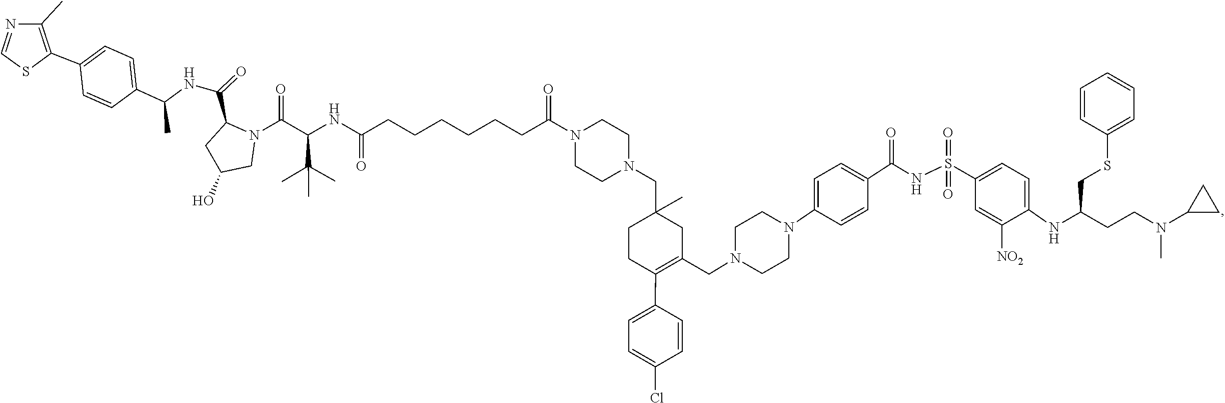 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C7CC7)CSc7ccccc7)c([N+](=O)[O-])c6)cc5)CC4)C3)CC2)C(C)(C)C)cc1] |
| 36 | 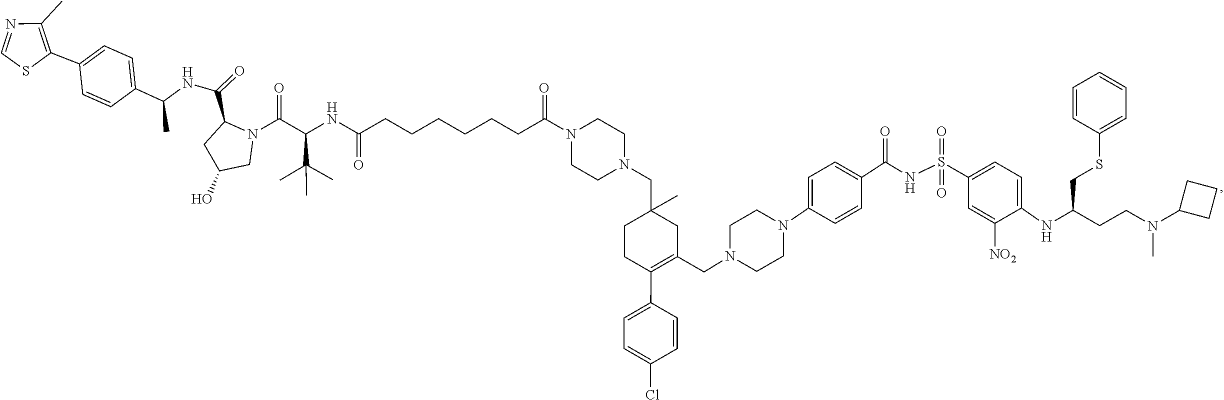 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C7CCC7)CSc7ccccc7)c([N+](=O)[O-])c6)cc5)CC4)C3)CC2)C(C)(C)C)cc1] |
| 37 | 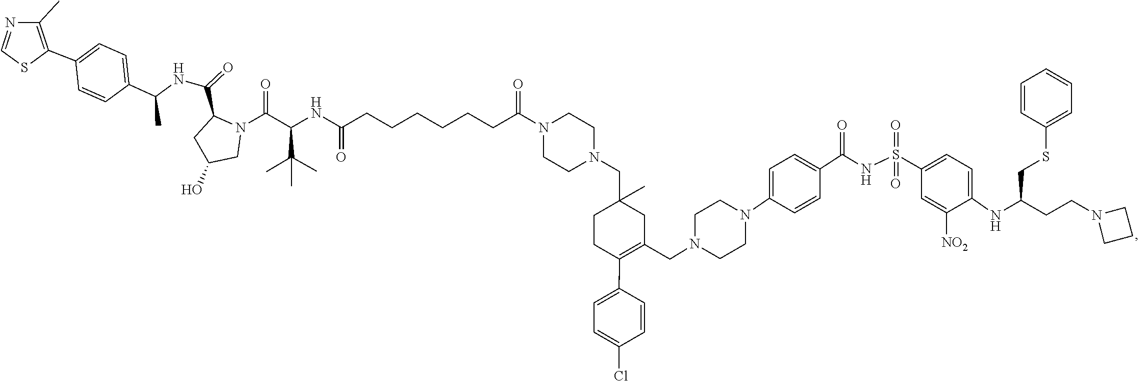 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCC7)CSc7ccccc7)c([N+](=O)[O-])c6)cc5)CC4)C3)CC2)C(C)(C)C)cc1] |
| 38 | 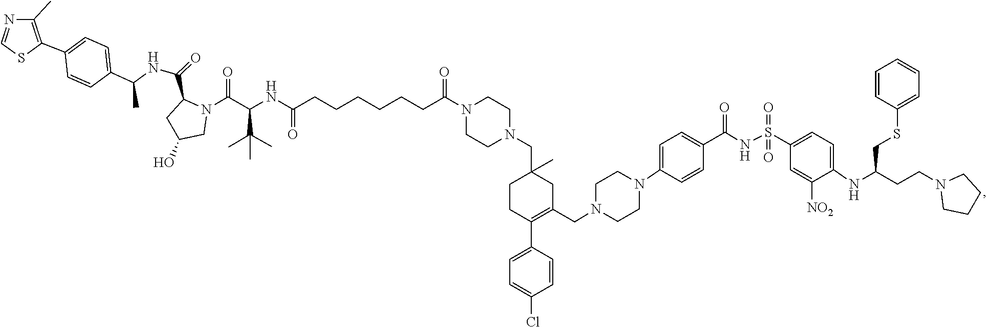 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCCC7)CSc7ccccc7)c([N+](=O)[O-])c6)cc5)CC4)C3)CC2)C(C)(C)C)cc1] |
| 39 | 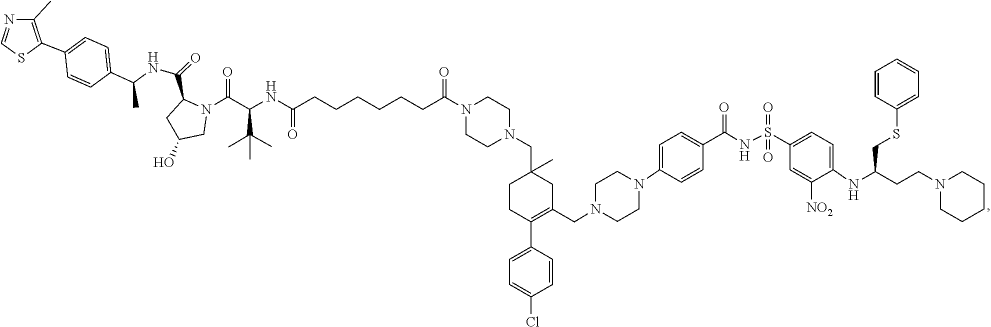 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCCCC7)CSc7ccccc7)c([N+](=O)[O-])c6)cc5)CC4)C3)CC2)C(C)(C)C)cc1] |
| 40 | 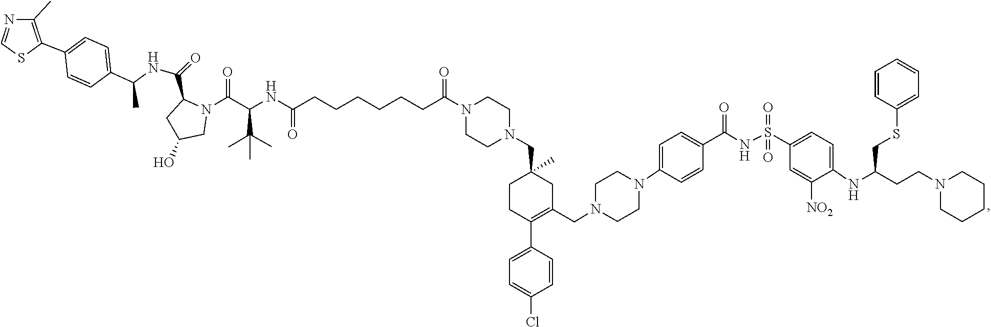 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCC7)CSc7ccccc7)c([N+](=O)[O-])c6)cc5)CC4)C3)CC2)C(C)(C)C)cc1] |
| 41 | 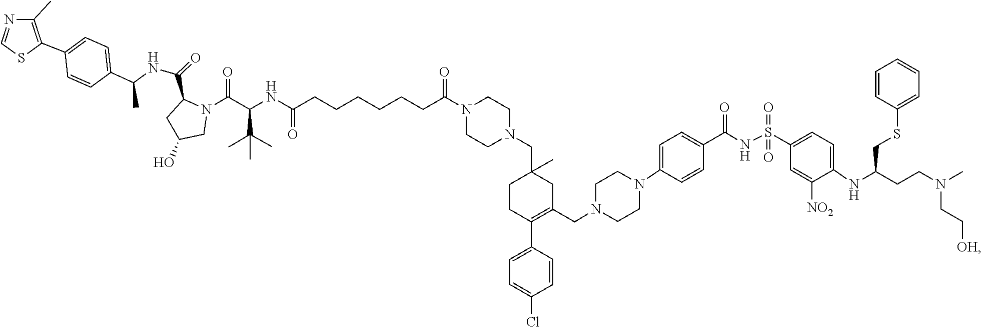 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)CCO)CSc7ccccc7)c([N+](=O)[O-])c6)cc5)CC4)C3)CC2)C(C)(C)C)cc1] |
| 42 | 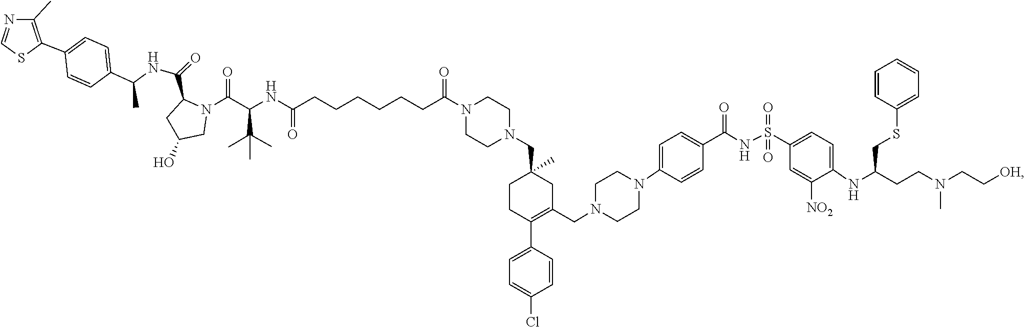 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN(C)CCO)CSc7ccccc7)c([N+](=O)[O-])c6)cc5)CC4)C3)CC2)C(C)(C)C)cc1] |
| 43 | 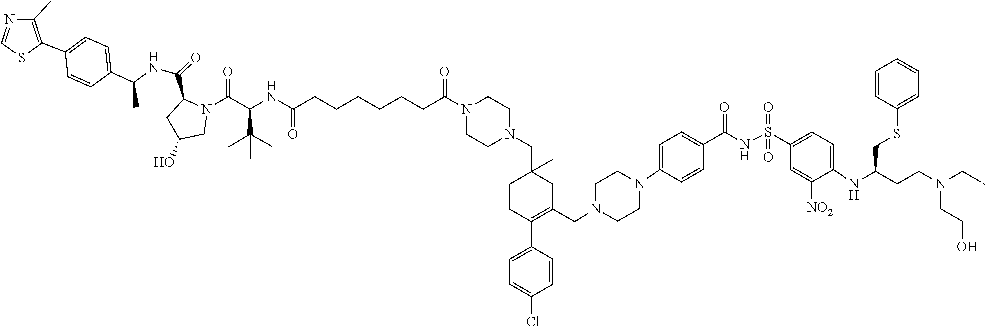 |
| 44 | 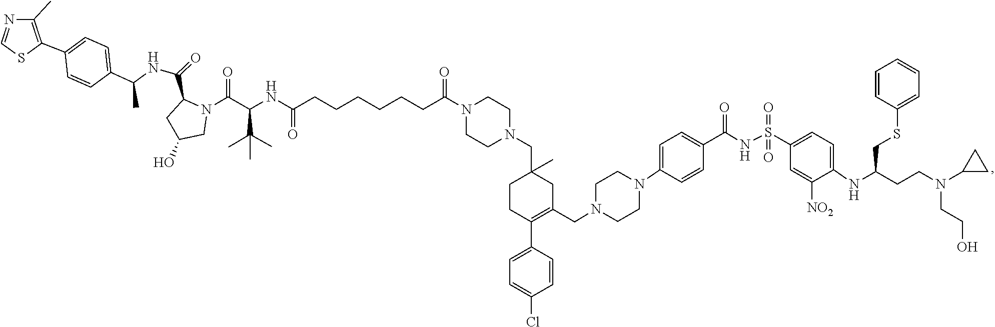 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(CCO)C7CC7)CSc7ccccc7)c([N+](=O)[O-])c6)cc5)CC4)C3)CC2)C(C)(C)C)cc1] |
| 45 | 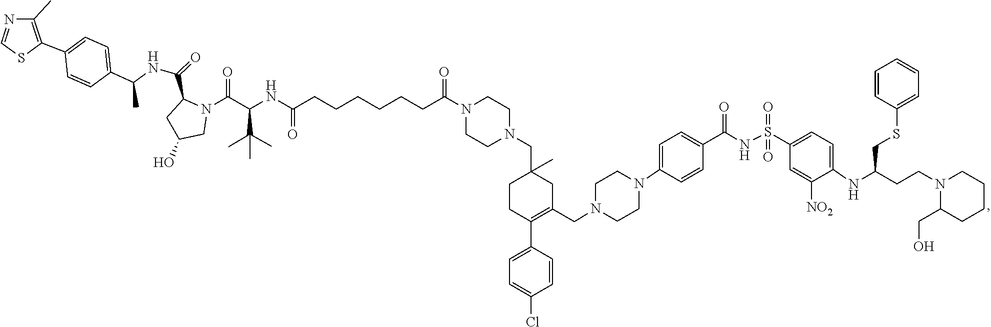 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCCCC7CO)CSc7ccccc7)c([N+](=O)[O-])c6)cc5)CC4)C3)CC2)C(C)(C)C)cc1] |
| 46 | 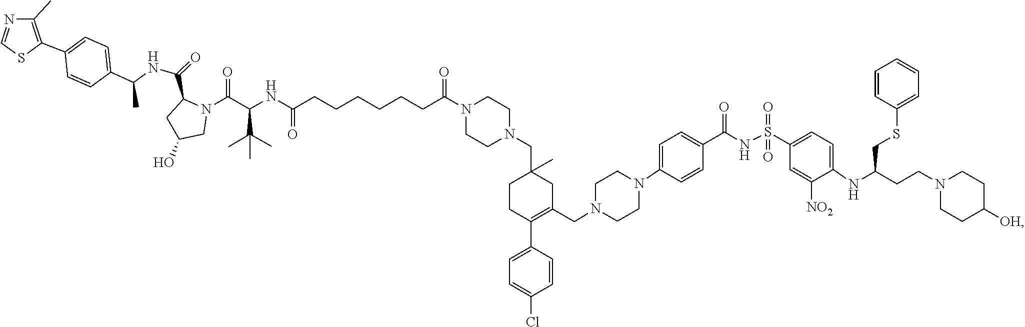 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCC(O)CC7)CSc7ccccc7)c([N+](=O)[O-])c6)cc5)CC4)C3)CC2)C(C)(C)C)cc1] |
| 47 | 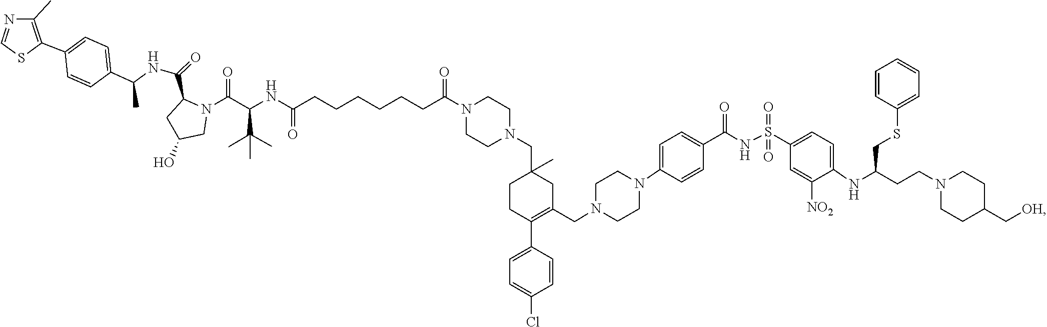 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCC(CO)CC7)CSc7ccccc7)c([N+](=O)[O-])c6)cc5)CC4)C3)CC2)C(C)(C)C)cc1] |
| 48 | 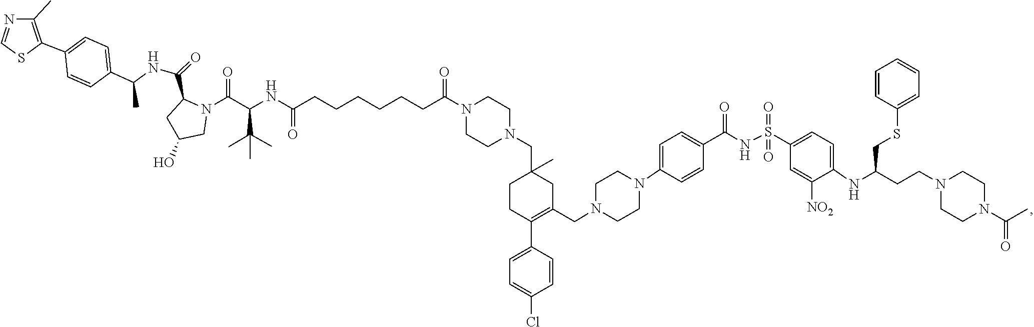 [SMILES: CC(=O)N1CCN(CC[C@H](CSc2ccccc2)Nc2ccc(S(=O)(=O)NC(=O)c3ccc(N4CCN(CC5=C(c6ccc(Cl)cc6)CCC(C)(CN6CCN(C(=O)CCCCCCC(=O)N[C@H](C(=O)N7C[C@H](O)C[C@H]7C(=O)N[C@@H](C)c7ccc(-c8scnc8C)cc7)C(C)(C)C)CC6)C5)CC4)cc3)cc2[N+](=O)[O-])CC1] |
| 49 | 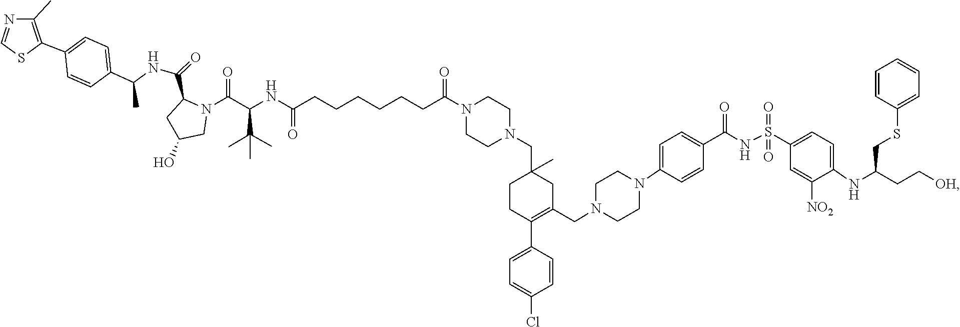 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCO)CSc7ccccc7)c([N+](=O)[O-])c6)cc5)CC4)C3)CC2)C(C)(C)C)cc1] |
| 50 | 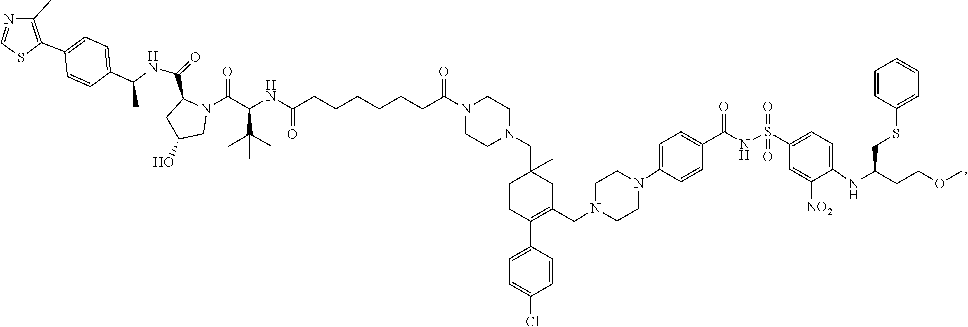 [SMILES: COCC[C@H](CSc1ccccc1)Nc1ccc(S(=O)(=O)NC(=O)c2ccc(N3CCN(CC4=C(c5ccc(Cl)cc5)CCC(C)(CN5CCN(C(=O)CCCCCCC(=O)N[C@H](C(=O)N6C[C@H](O)C[C@H]6C(=O)N[C@@H](C)c6ccc(-c7scnc7C)cc6)C(C)(C)C)CC5)C4)CC3)cc2)cc1[N+](=O)[O-]] |
| 51 | 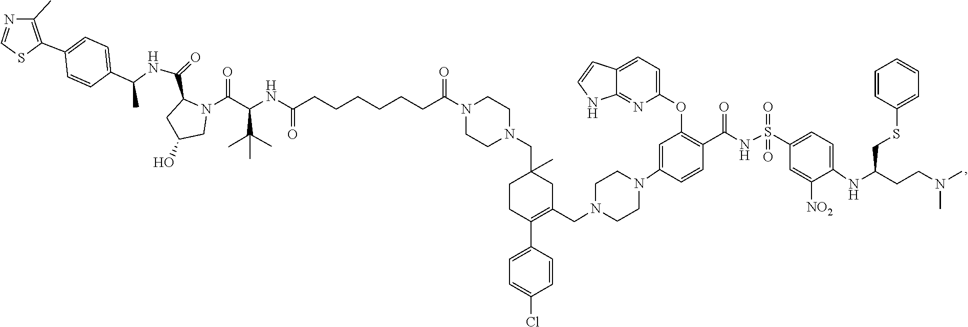 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c([N+](=O)[O-])c6)c(Oc6ccc7cc[nH]c7n6)c5)CC4)C3)CC2)C(C)(C)C)cc1] |
| 52 | 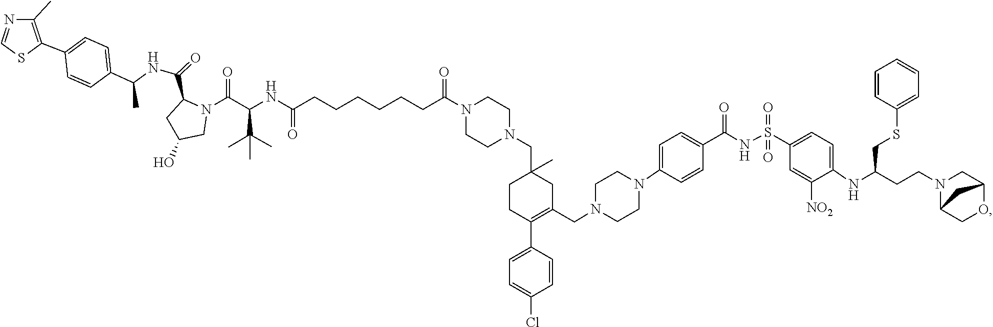 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c([N+](=O)[O-])c6)cc5)CC4)C3)CC2)C(C)(C)C)cc1] |
| 53 | 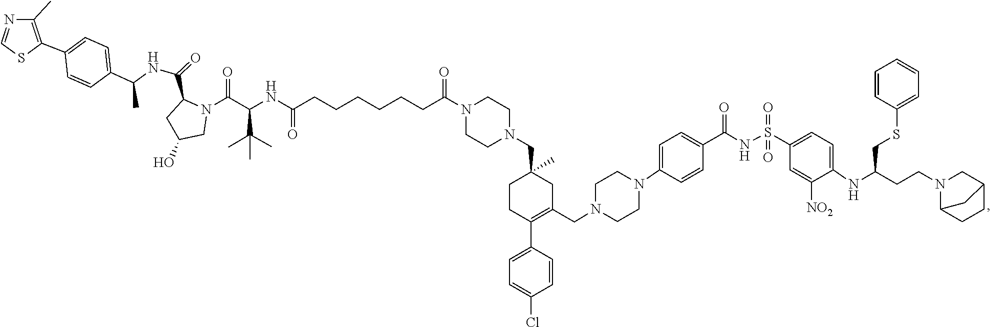 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CC8CCC7C8)CSc7ccccc7)c([N+](=O)[O-])c6)cc5)CC4)C3)CC2)C(C)(C)C)cc1] |
| 54 | 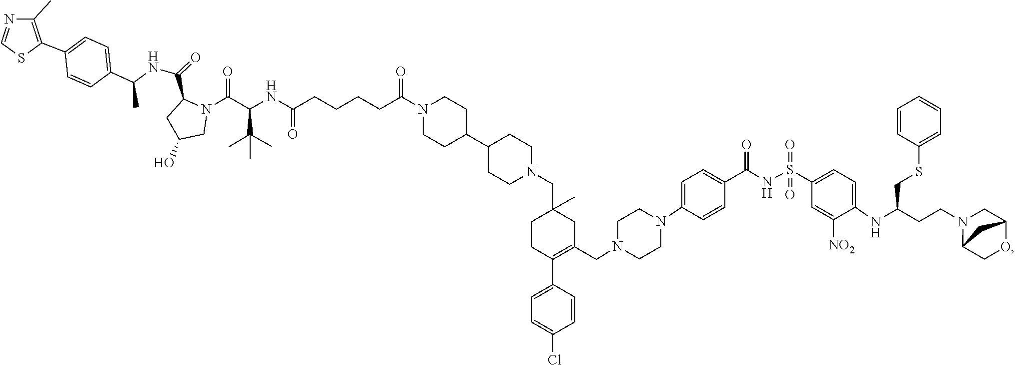 [SMILES: Cc1ncsc1-c1ccc([C@H](C)NC(=O)[C@@H]2C[C@@H](O)CN2C(=O)[C@@H](NC(=O)CCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c([N+](=O)[O-])c7)cc6)CC5)C4)CC3)CC2)C(C)(C)C)cc1] |
| 55 | 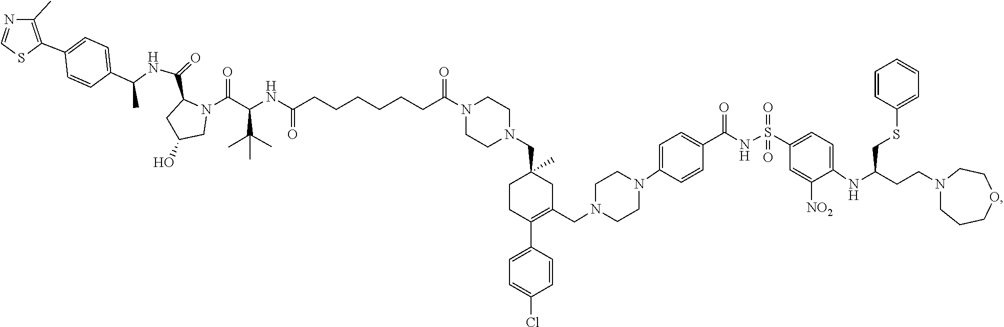 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c([N+](=O)[O-])c6)cc5)CC4)C3)CC2)C(C)(C)C)cc1] |
| 56 | 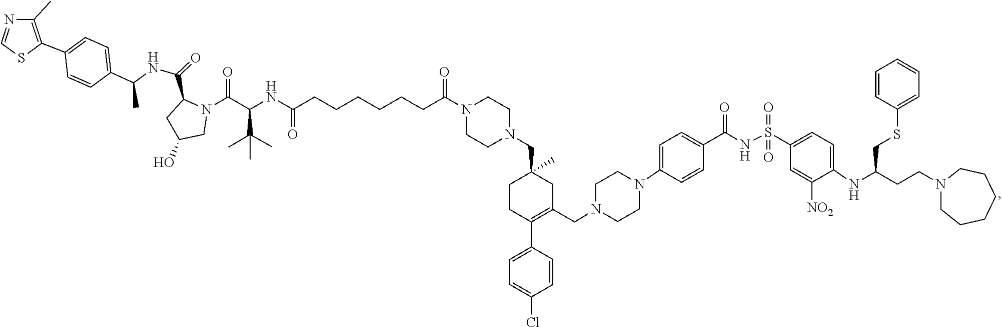 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCCC7)CSc7ccccc7)c([N+](=O)[O-])c6)cc5)CC4)C3)CC2)C(C)(C)C)cc1] |
| 57 | 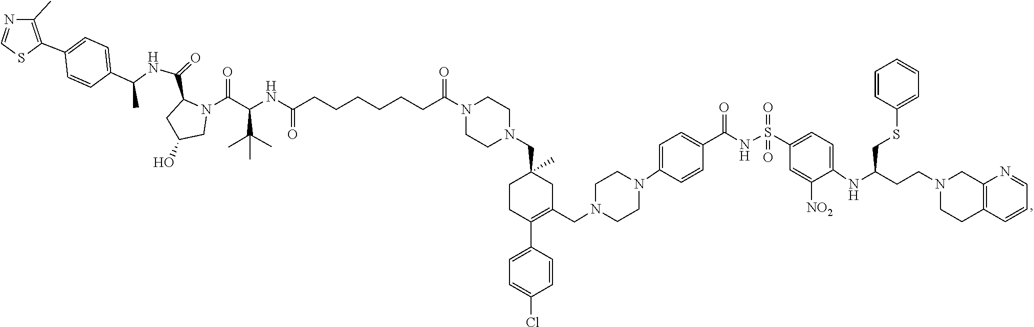 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCc8cccnc8C7)CSc7ccccc7)c([N+](=O)[O-])c6)cc5)CC4)C3)CC2)C(C)(C)C)cc1] |
| 58 | 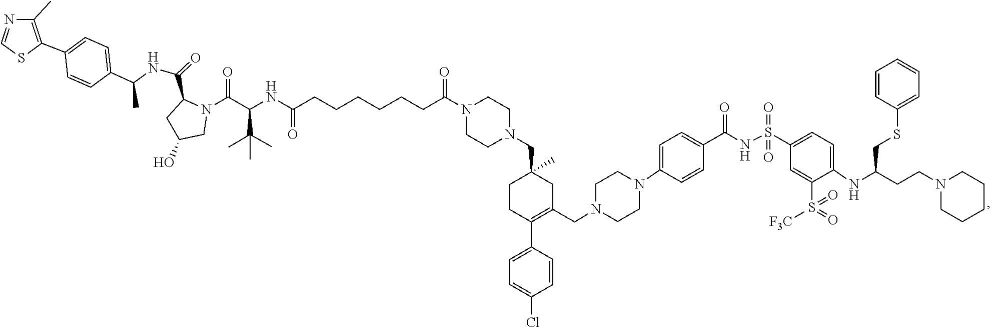 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCC7)CSc7ccccc7)c(S(=O)(=O)C(F)(F)F)c6)cc5)CC4)C3)CC2)C(C)(C)C)cc1] |
| 59 | 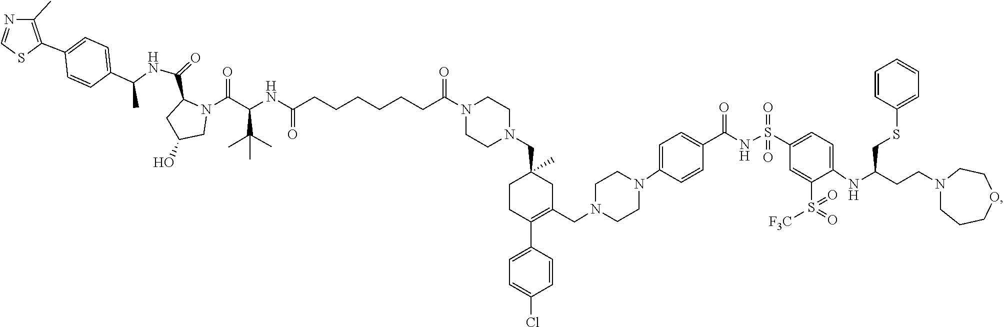 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)C(C)(C)C)cc1] |
| 60 | 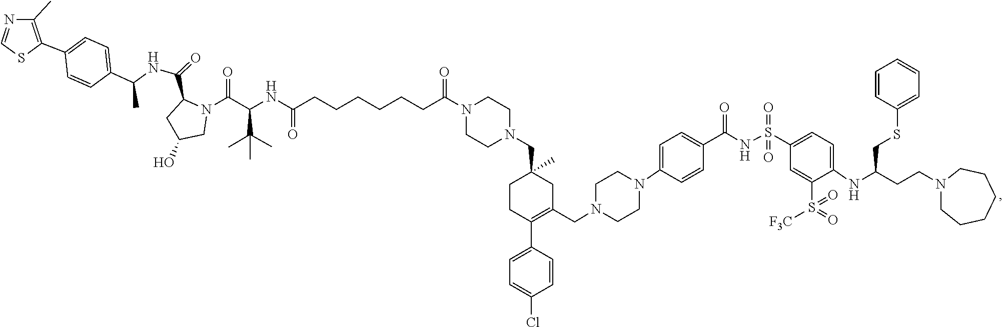 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCCC7)CSc7ccccc7)c(S(=O)(=O)C(F)(F)F)c6)cc5)CC4)C3)CC2)C(C)(C)C)cc1] |
| 61 | 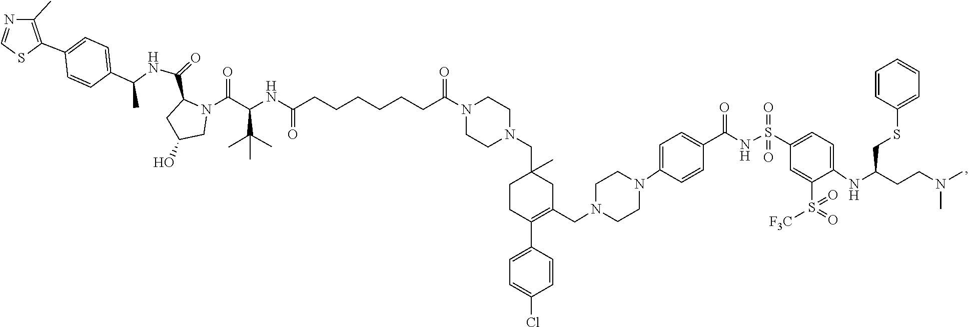 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c(S(=O)(=O)C(F)(F)F)c6)cc5)CC4)C3)CC2)C(C)(C)C)cc1] |
| 62 | 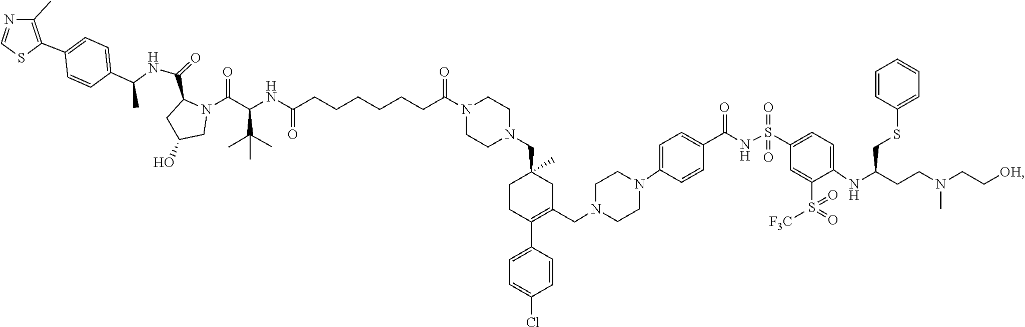 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN(C)CCO)CSc7ccccc7)c(S(=O)(=O)C(F)(F)F)c6)cc5)CC4)C3)CC2)C(C)(C)C)cc1] |
| 63 | 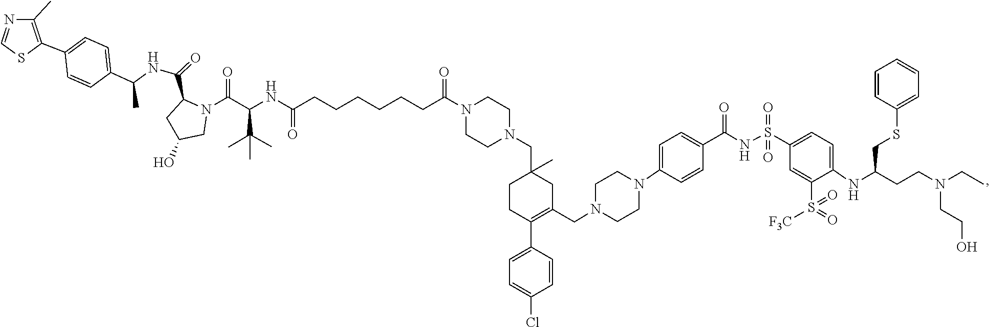 [SMILES: CCN(CCO)CC[C@H](CSc1ccccc1)Nc1ccc(S(=O)(=O)NC(=O)c2ccc(N3CCN(CC4=C(c5ccc(Cl)cc5)CCC(C)(CN5CCN(C(=O)CCCCCCC(=O)N[C@H](C(=O)N6C[C@H](O)C[C@H]6C(=O)N[C@@H](C)c6ccc(-c7scnc7C)cc6)C(C)(C)C)CC5)C4)CC3)cc2)cc1S(=O)(=O)C(F)(F)F] |
| 64 | 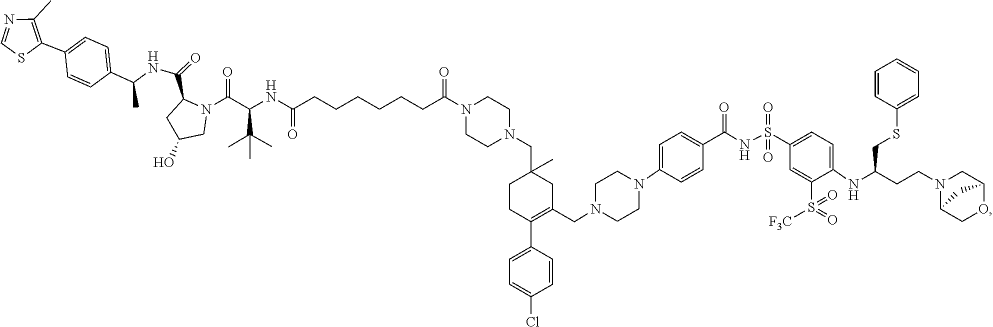 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@@H]8C[C@H]7CO8)CSc7ccccc7)c(S(=O)(=O)C(F)(F)F)c6)cc5)CC4)C3)CC2)C(C)(C)C)cc1] |
| 64 | 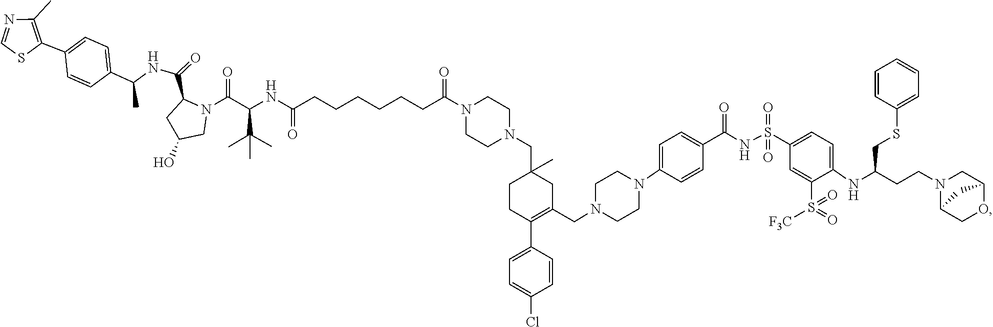 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@@H]8C[C@H]7CO8)CSc7ccccc7)c(S(=O)(=O)C(F)(F)F)c6)cc5)CC4)C3)CC2)C(C)(C)C)cc1] |
| 65 | 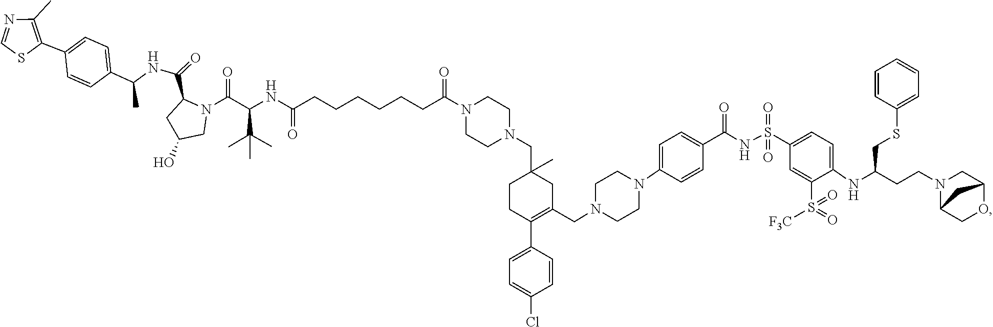 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c(S(=O)(=O)C(F)(F)F)c6)cc5)CC4)C3)CC2)C(C)(C)C)cc1] |
| 66 | 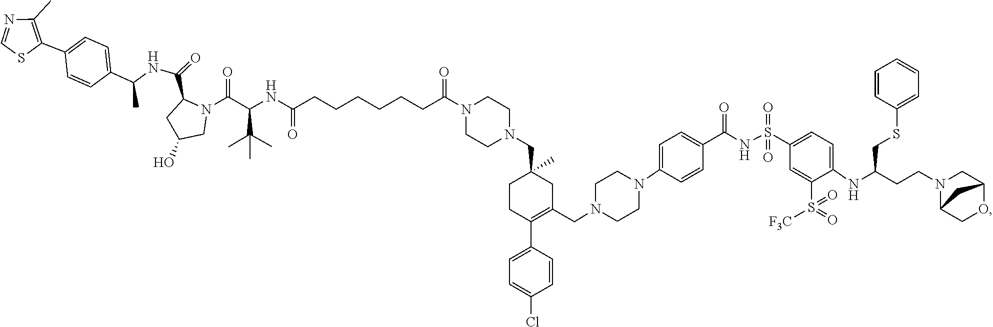 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c(S(=O)(=O)C(F)(F)F)c6)cc5)CC4)C3)CC2)C(C)(C)C)cc1] |
| 67 | 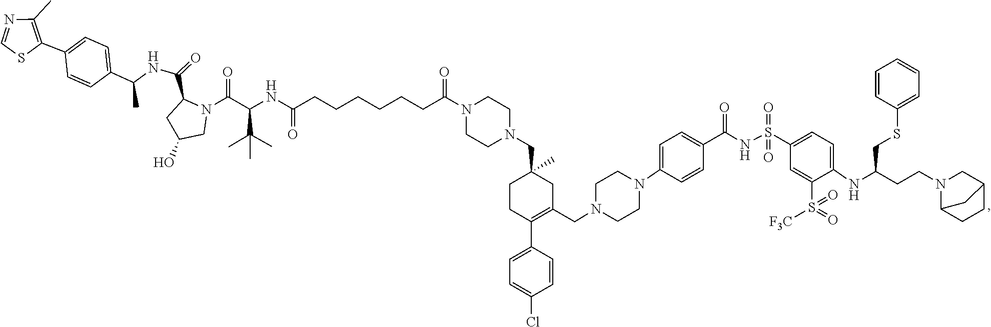 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CC8CCC7C8)CSc7ccccc7)c(S(=O)(=O)C(F)(F)F)c6)cc5)CC4)C3)CC2)C(C)(C)C)cc1] |
| 68 | 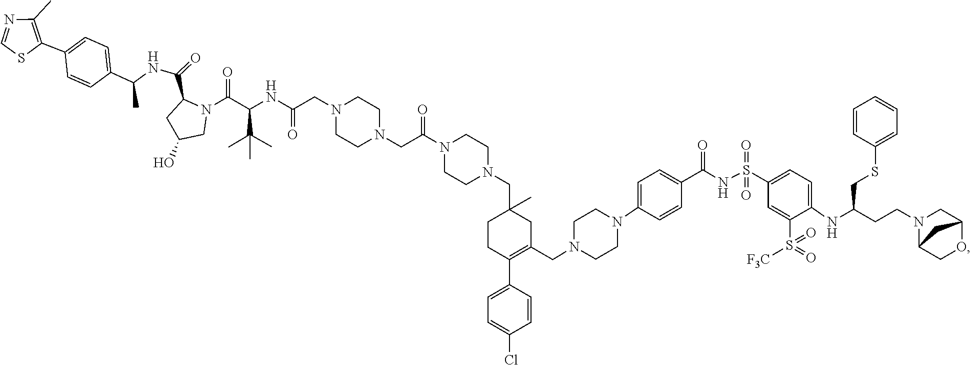 [SMILES: Cc1ncsc1-c1ccc([C@H](C)NC(=O)[C@@H]2C[C@@H](O)CN2C(=O)[C@@H](NC(=O)CN2CCN(CC(=O)N3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1] |
| 69 | 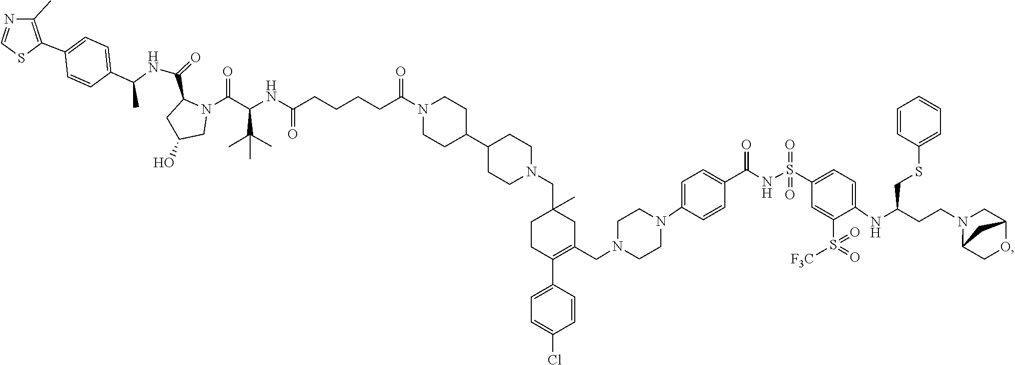 [SMILES: Cc1ncsc1-c1ccc([C@H](C)NC(=O)[C@@H]2C[C@@H](O)CN2C(=O)[C@@H](NC(=O)CCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1] |
| 70 | 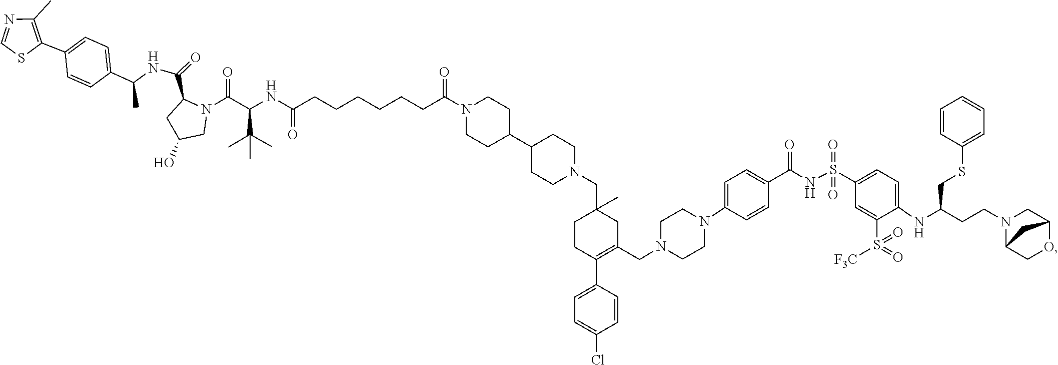 [SMILES: Cc1ncsc1-c1ccc([C@H](C)NC(=O)[C@@H]2C[C@@H](O)CN2C(=O)[C@@H](NC(=O)CCCCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1] |
| 71 | 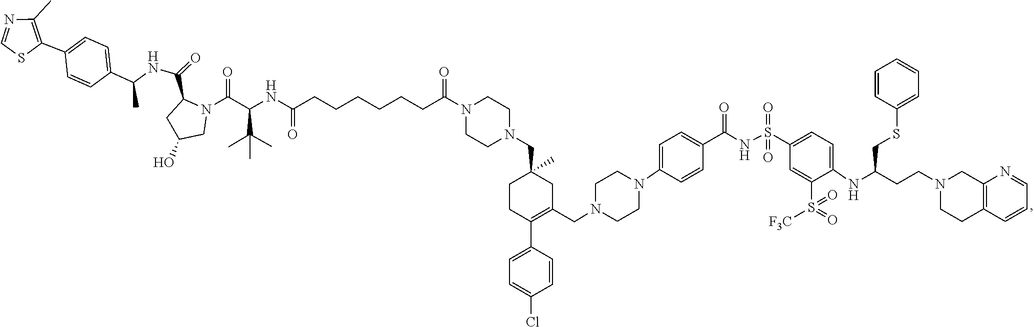 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCc8cccnc8C7)CSc7ccccc7)c(S(=O)(=O)C(F)(F)F)c6)cc5)CC4)C3)CC2)C(C)(C)C)cc1] |
| 72 | 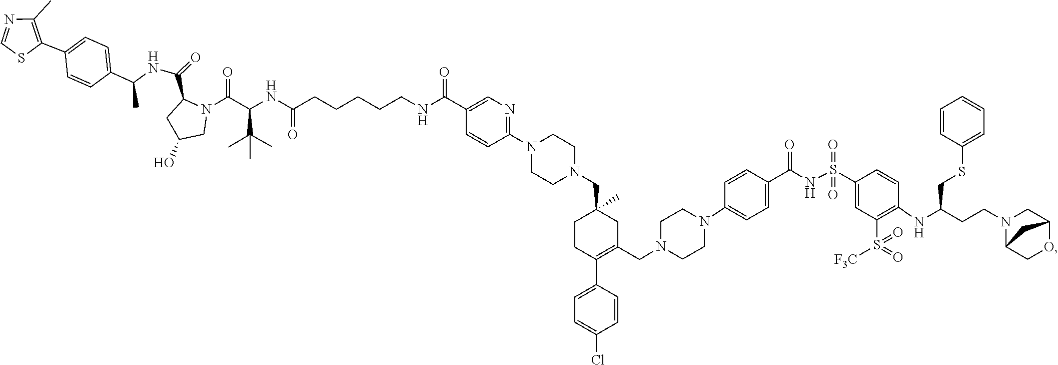 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)nc2)C(C)(C)C)cc1] |
| 73 | 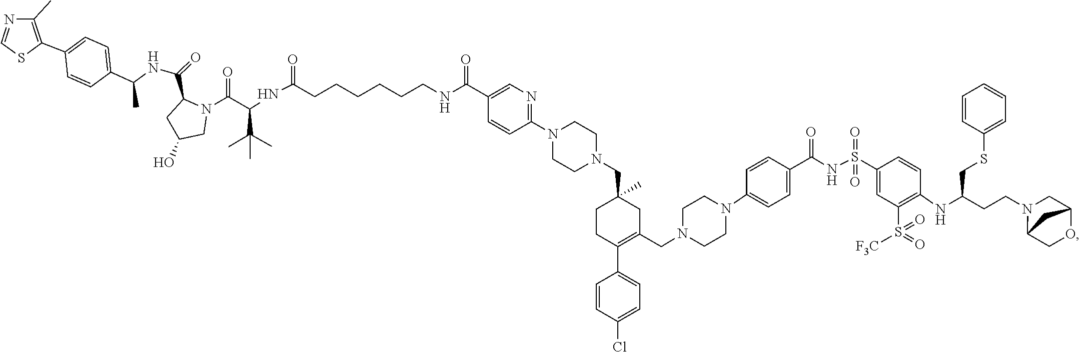 [SMILES: Cc1ncsc1-c1ccc([C@H](C)NC(=O)[C@@H]2C[C@@H](O)CN2C(=O)[C@@H](NC(=O)CCCCCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)nc2)C(C)(C)C)cc1] |
| 74 | 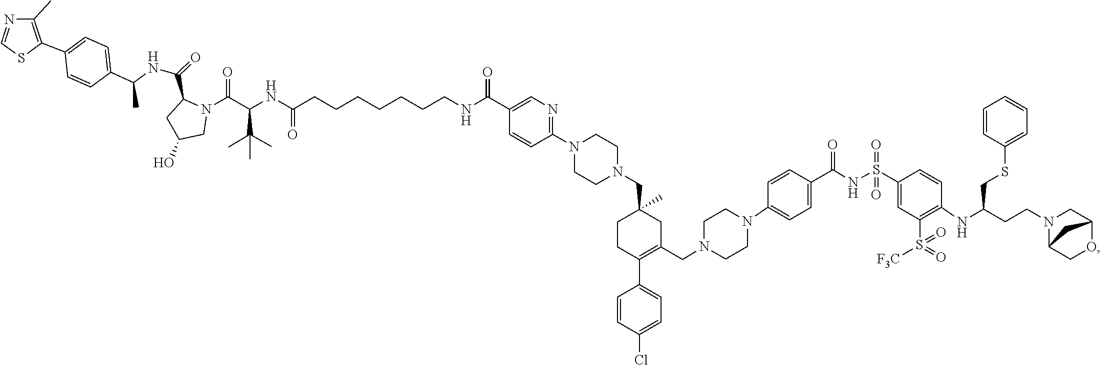 [SMILES: Cc1ncsc1-c1ccc([C@H](C)NC(=O)[C@@H]2C[C@@H](O)CN2C(=O)[C@@H](NC(=O)CCCCCCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)nc2)C(C)(C)C)cc1] |
| 75 | 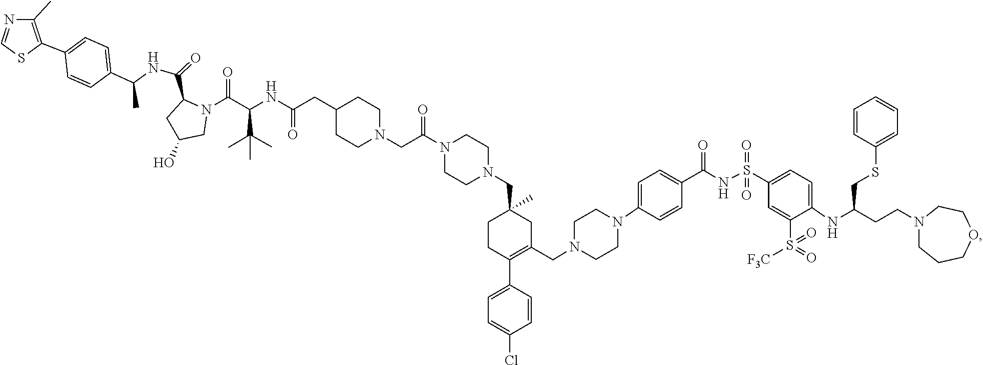 [SMILES: Cc1ncsc1-c1ccc([C@H](C)NC(=O)[C@@H]2C[C@@H](O)CN2C(=O)[C@@H](NC(=O)CC2CCN(CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 76 | 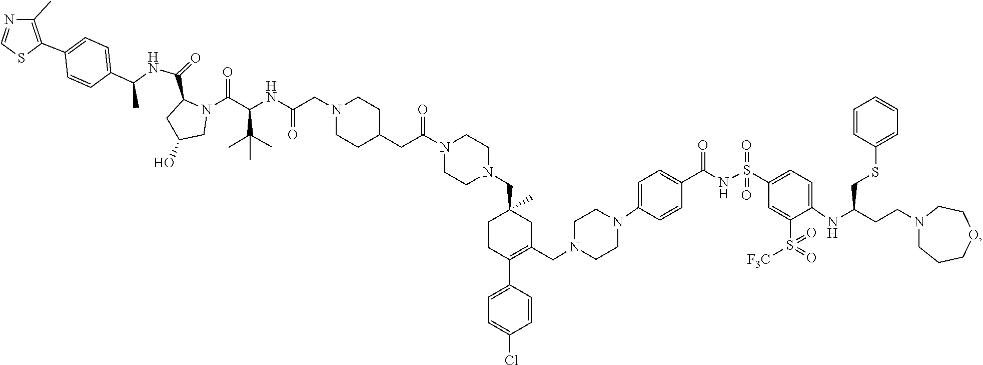 [SMILES: Cc1ncsc1-c1ccc([C@H](C)NC(=O)[C@@H]2C[C@@H](O)CN2C(=O)[C@@H](NC(=O)CN2CCC(CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 77 | 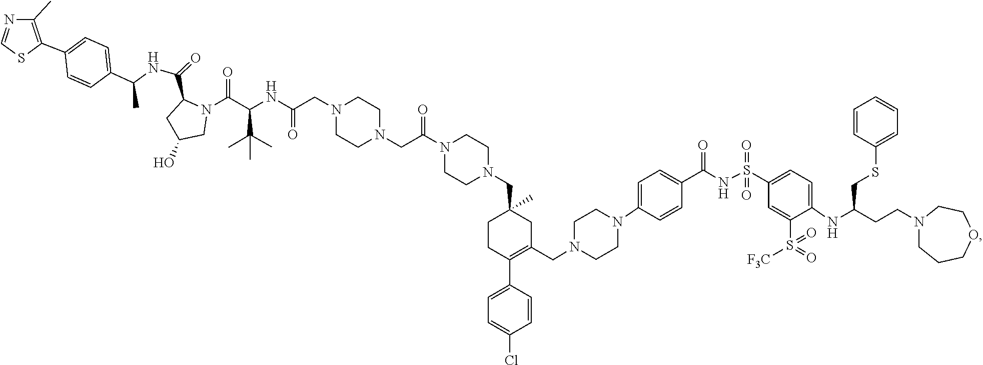 [SMILES: Cc1ncsc1-c1ccc([C@H](C)NC(=O)[C@@H]2C[C@@H](O)CN2C(=O)[C@@H](NC(=O)CN2CCN(CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 78 | 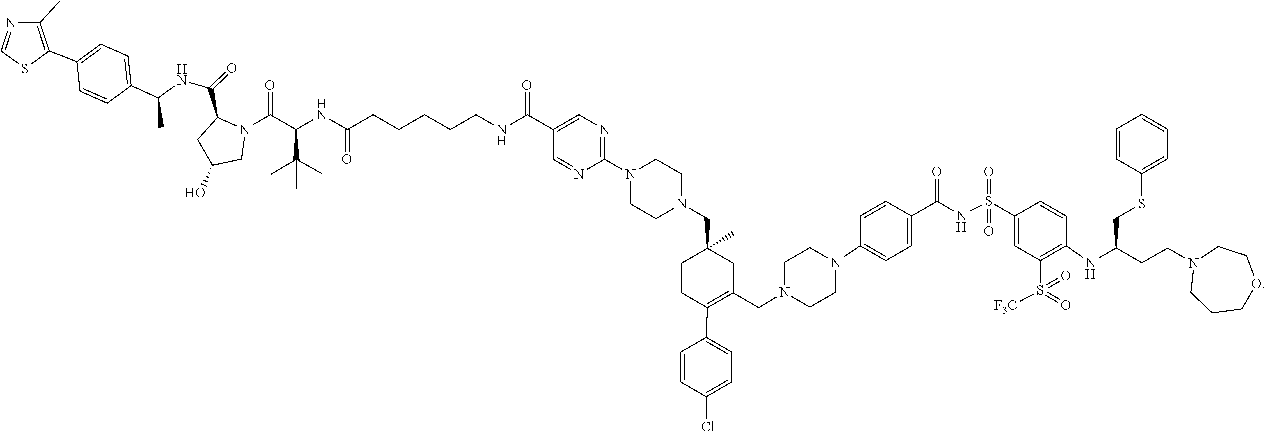 [SMILES: Cc1ncsc1-c1ccc([C@H](C)NC(=O)[C@@H]2C[C@@H](O)CN2C(=O)[C@@H](NC(=O)CCCCCNC(=O)c2cnc(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nc2)C(C)(C)C)cc1] |
| 79 | 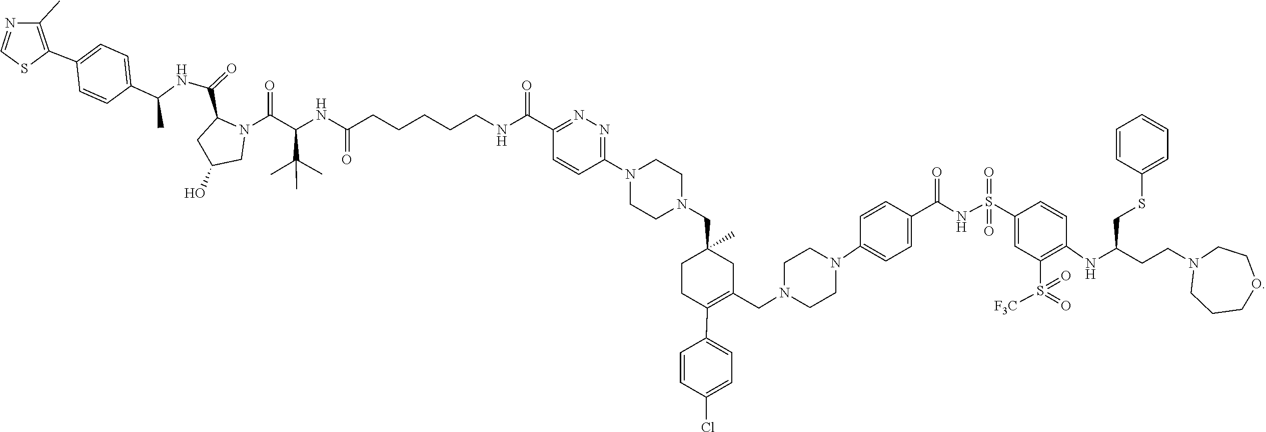 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nn2)C(C)(C)C)cc1] |
| 80 | 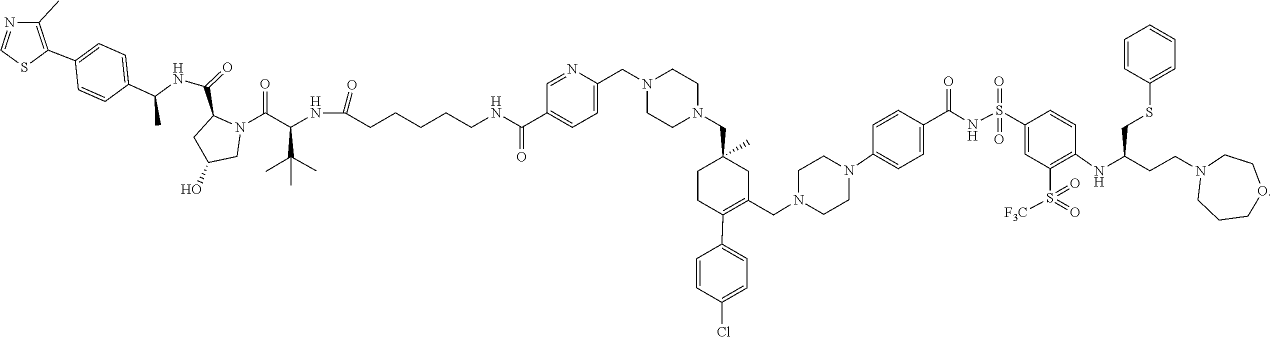 [SMILES: Cc1ncsc1-c1ccc([C@H](C)NC(=O)[C@@H]2C[C@@H](O)CN2C(=O)[C@@H](NC(=O)CCCCCNC(=O)c2ccc(CN3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nc2)C(C)(C)C)cc1] |
| 81 | 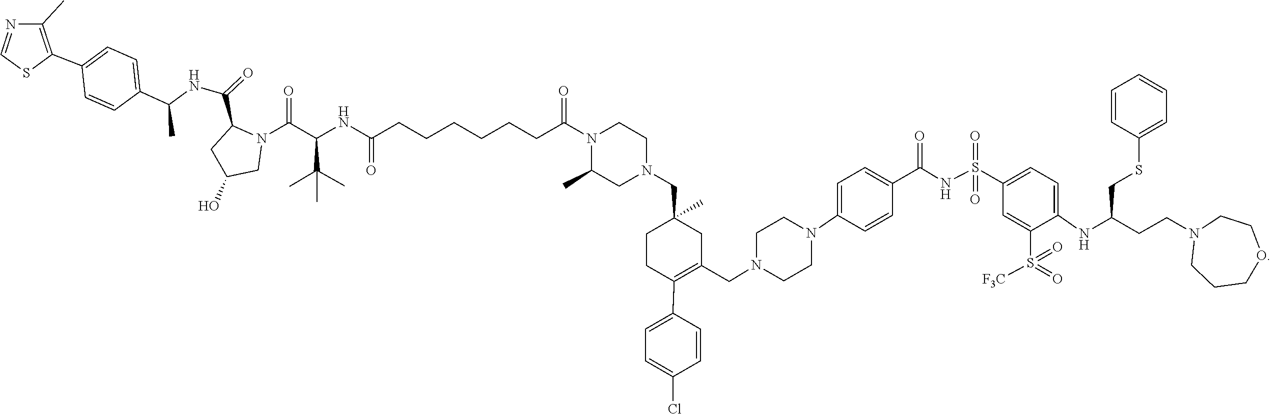 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)C[C@H]2C)C(C)(C)C)cc1] |
| 82 | 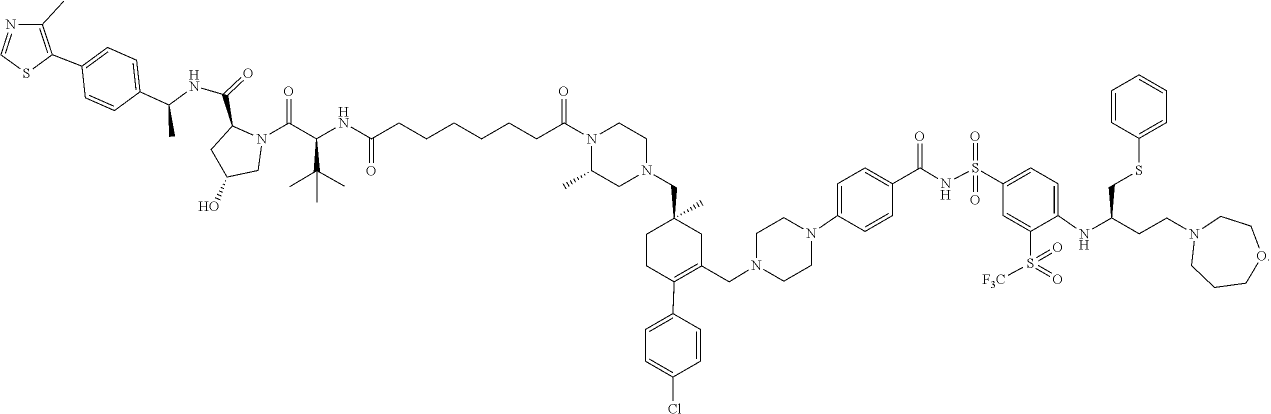 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)C[C@@H]2C)C(C)(C)C)cc1] |
| 83 | 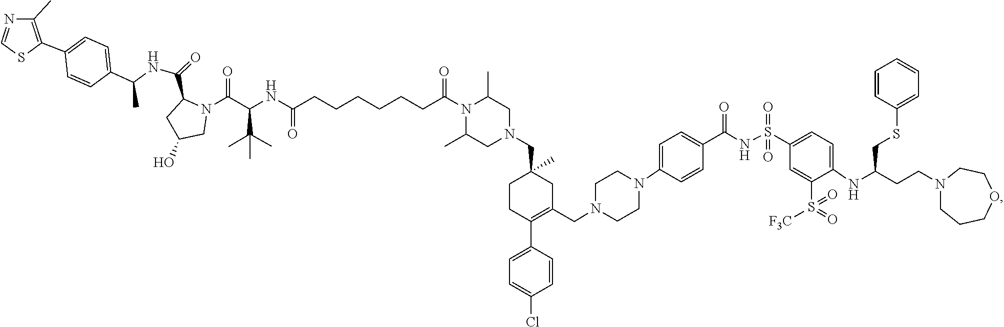 [SMILES: Cc1ncsc1-c1ccc([C@H](C)NC(=O)[C@@H]2C[C@@H](O)CN2C(=O)[C@@H](NC(=O)CCCCCCC(=O)N2C(C)CN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2C)C(C)(C)C)cc1] |
| 84 | 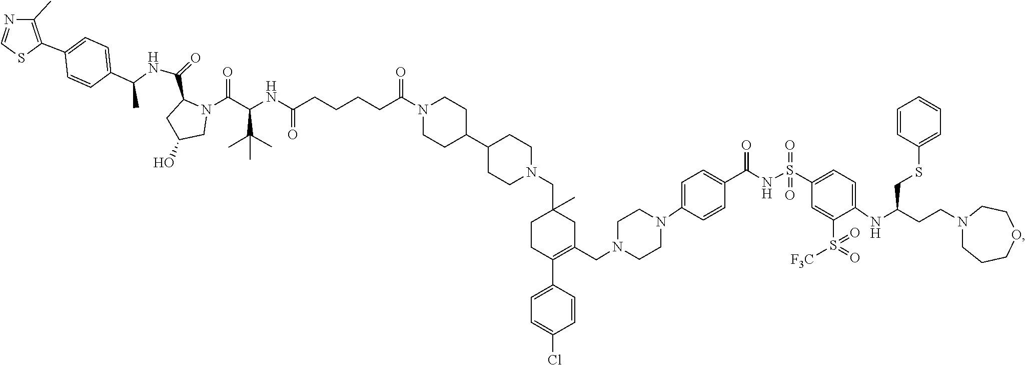 [SMILES: Cc1ncsc1-c1ccc([C@H](C)NC(=O)[C@@H]2C[C@@H](O)CN2C(=O)[C@@H](NC(=O)CCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 85 | 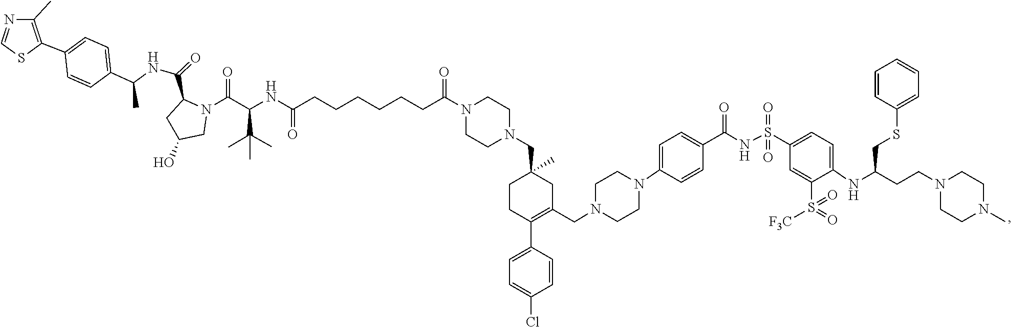 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCN(C)CC7)CSc7ccccc7)c(S(=O)(=O)C(F)(F)F)c6)cc5)CC4)C3)CC2)C(C)(C)C)cc1] |
| 86 | 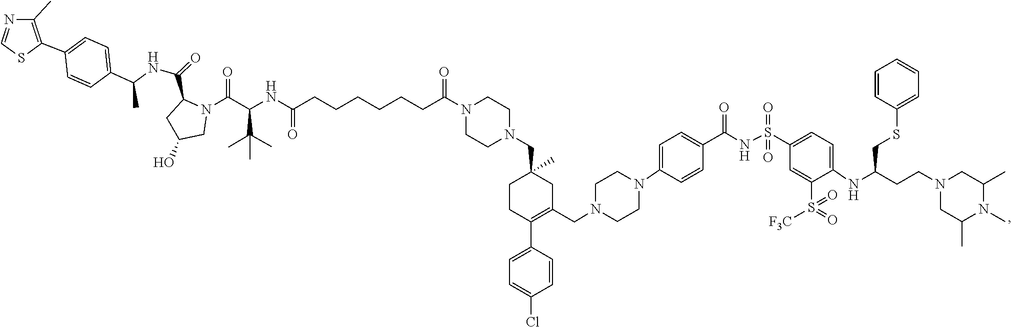 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CC(C)N(C)C(C)C7)CSc7ccccc7)c(S(=O)(=O)C(F)(F)F)c6)cc5)CC4)C3)CC2)C(C)(C)C)cc1] |
| 87 | 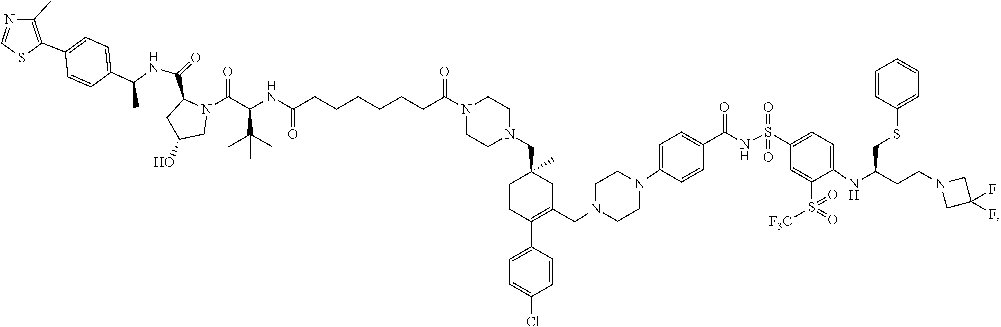 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CC(F)(F)C7)CSc7ccccc7)c(S(=O)(=O)C(F)(F)F)c6)cc5)CC4)C3)CC2)C(C)(C)C)cc1] |
| 88 | 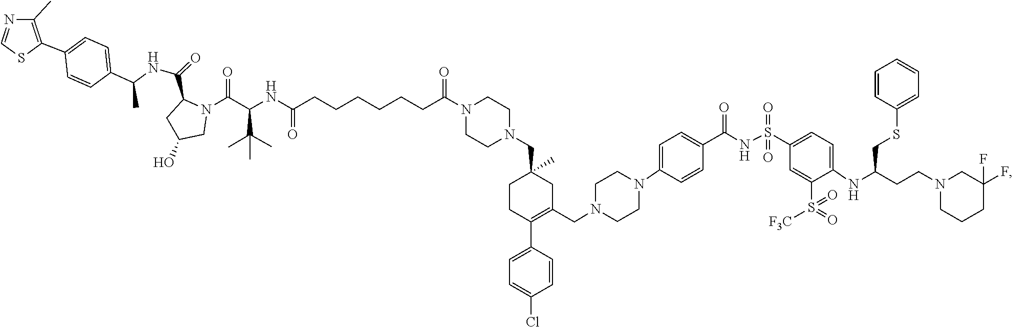 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCC(F)(F)C7)CSc7ccccc7)c(S(=O)(=O)C(F)(F)F)c6)cc5)CC4)C3)CC2)C(C)(C)C)cc1] |
| 89 | 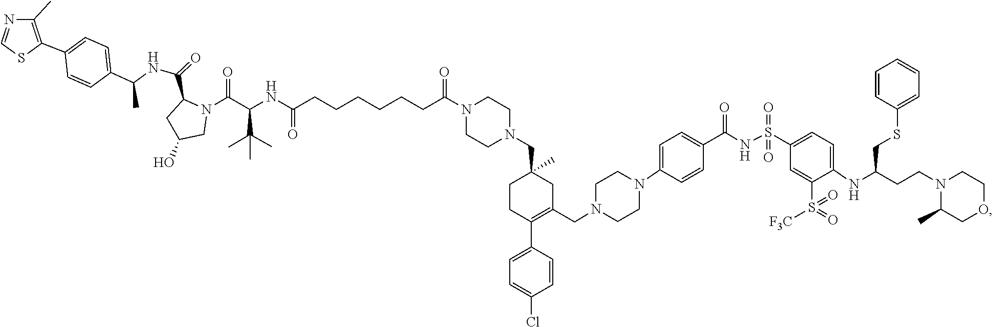 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC[C@H]7C)CSc7ccccc7)c(S(=O)(=O)C(F)(F)F)c6)cc5)CC4)C3)CC2)C(C)(C)C)cc1] |
| 90 | 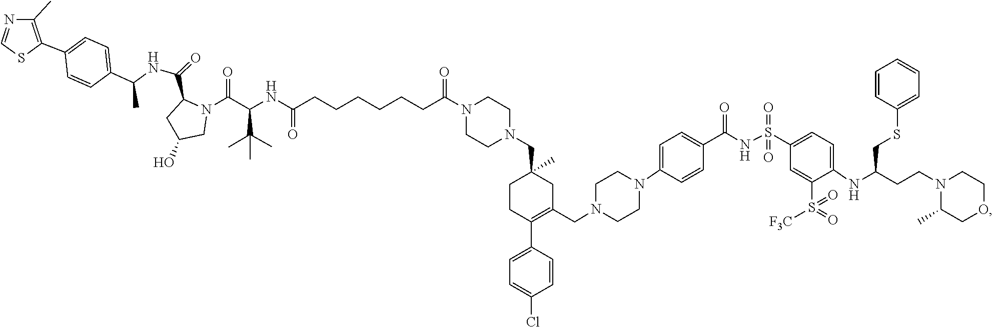 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC[C@@H]7C)CSc7ccccc7)c(S(=O)(=O)C(F)(F)F)c6)cc5)CC4)C3)CC2)C(C)(C)C)cc1] |
| 91 | 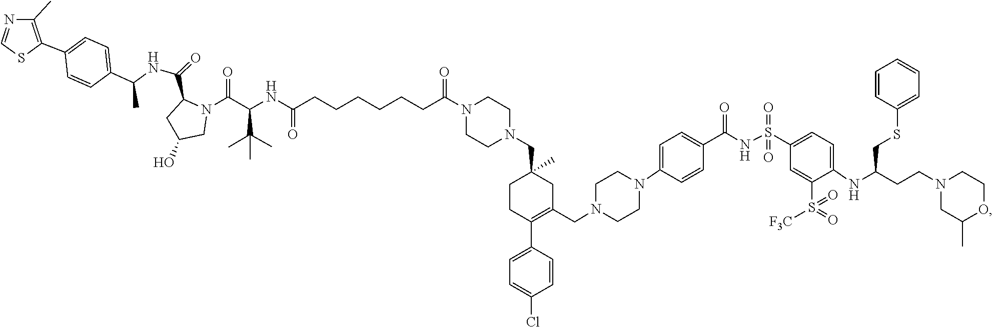 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC(C)C7)CSc7ccccc7)c(S(=O)(=O)C(F)(F)F)c6)cc5)CC4)C3)CC2)C(C)(C)C)cc1] |
| 92 | 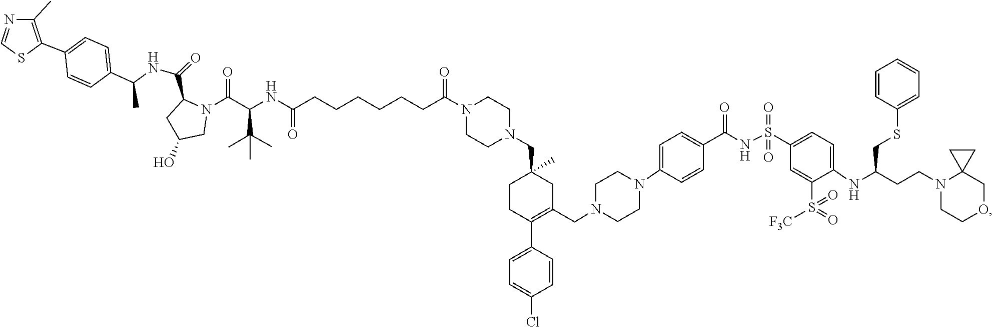 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC78CC8)CSc7ccccc7)c(S(=O)(=O)C(F)(F)F)c6)cc5)CC4)C3)CC2)C(C)(C)C)cc1] |
| 93 | 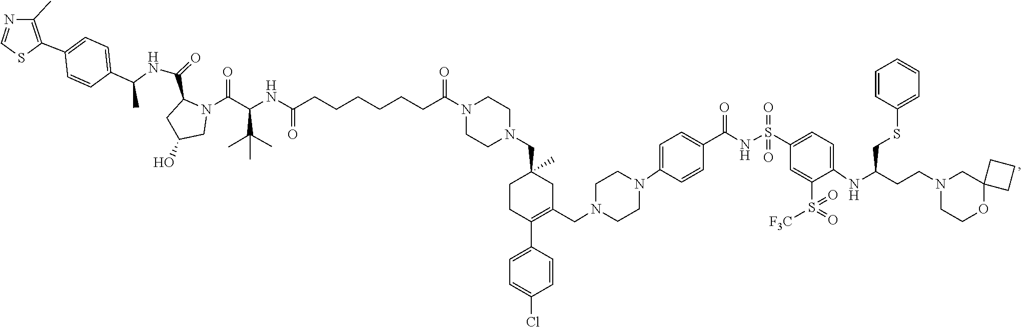 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC8(CCC8)C7)CSc7ccccc7)c(S(=O)(=O)C(F)(F)F)c6)cc5)CC4)C3)CC2)C(C)(C)C)cc1] |
| 94 | 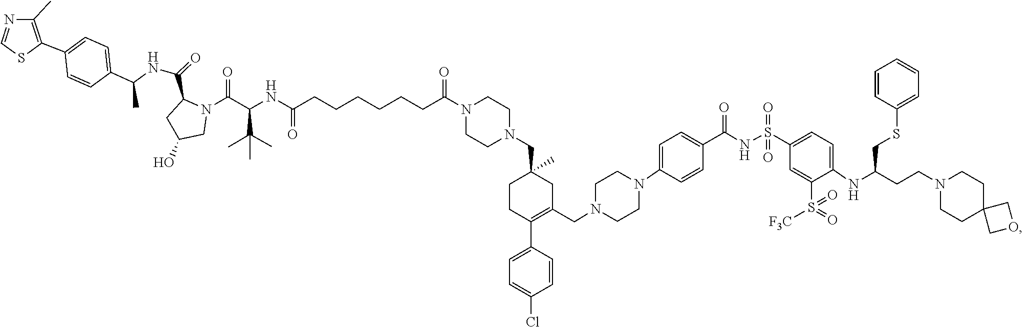 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCC8(CC7)COC8)CSc7ccccc7)c(S(=O)(=O)C(F)(F)F)c6)cc5)CC4)C3)CC2)C(C)(C)C)cc1] |
| 95 | 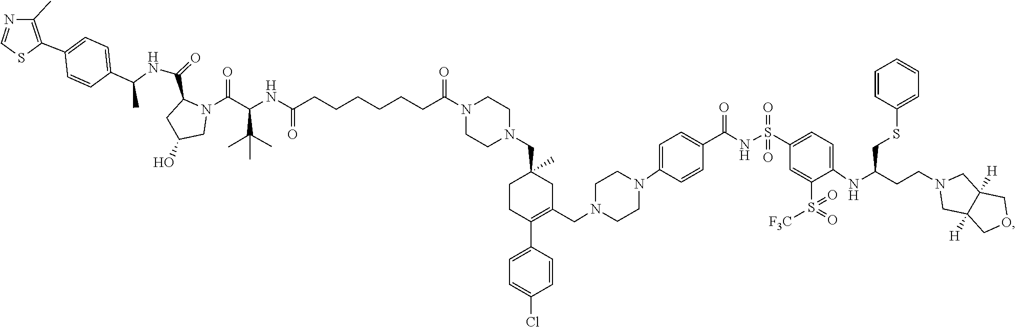 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7C[C@H]8COC[C@H]8C7)CSc7ccccc7)c(S(=O)(=O)C(F)(F)F)c6)cc5)CC4)C3)CC2)C(C)(C)C)cc1] |
| 96 | 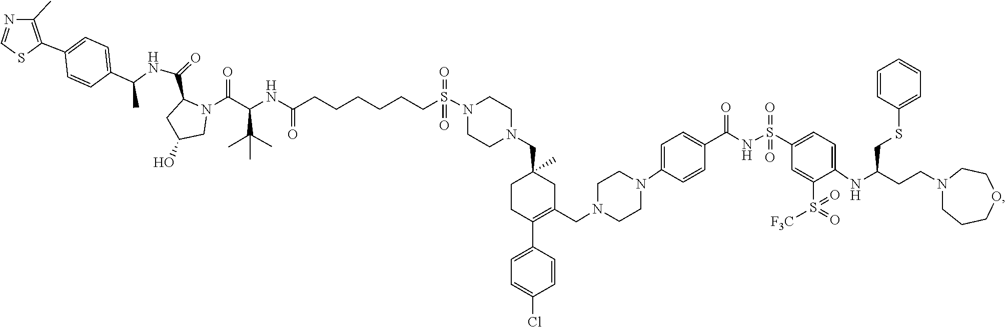 [SMILES: Cc1ncsc1-c1ccc([C@H](C)NC(=O)[C@@H]2C[C@@H](O)CN2C(=O)[C@@H](NC(=O)CCCCCCS(=O)(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)C(C)(C)C)cc1] |
| 97 | 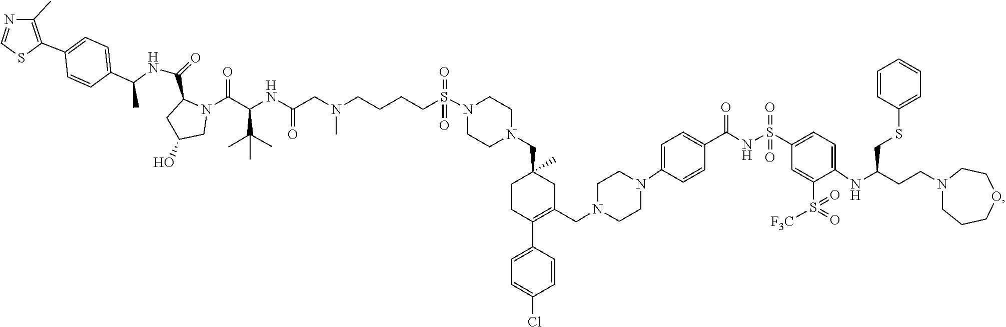 [SMILES: Cc1ncsc1-c1ccc([C@H](C)NC(=O)[C@@H]2C[C@@H](O)CN2C(=O)[C@@H](NC(=O)CN(C)CCCCS(=O)(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)C(C)(C)C)cc1] |
| 98 | 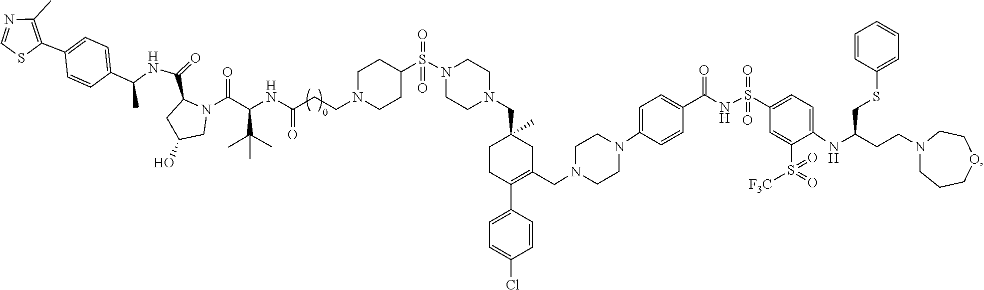 |
| 99 | 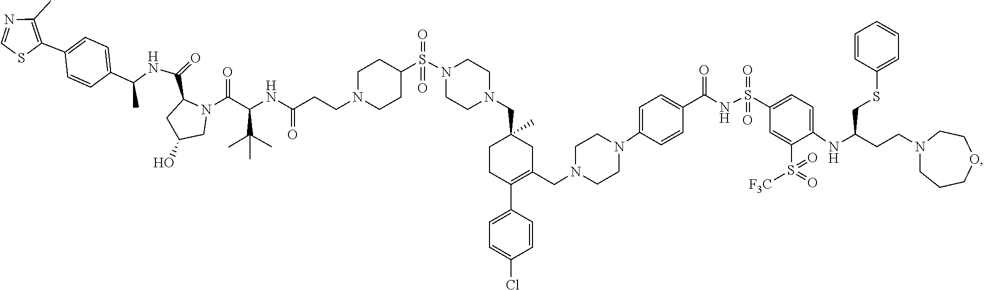 [SMILES: Cc1ncsc1-c1ccc([C@H](C)NC(=O)[C@@H]2C[C@@H](O)CN2C(=O)[C@@H](NC(=O)CCN2CCC(S(=O)(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 100 | 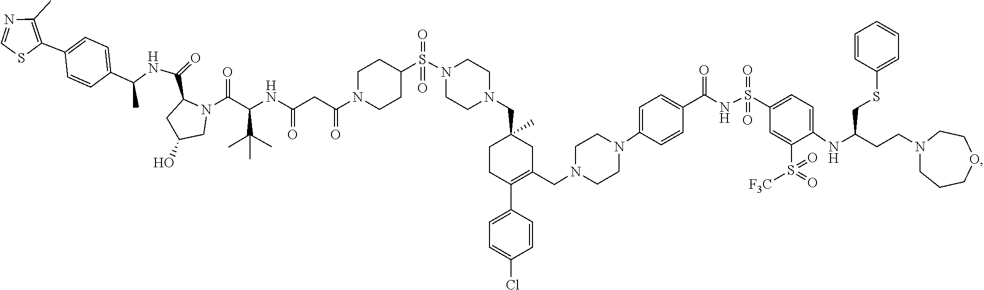 [SMILES: Cc1ncsc1-c1ccc([C@H](C)NC(=O)[C@@H]2C[C@@H](O)CN2C(=O)[C@@H](NC(=O)CC(=O)N2CCC(S(=O)(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 101 | 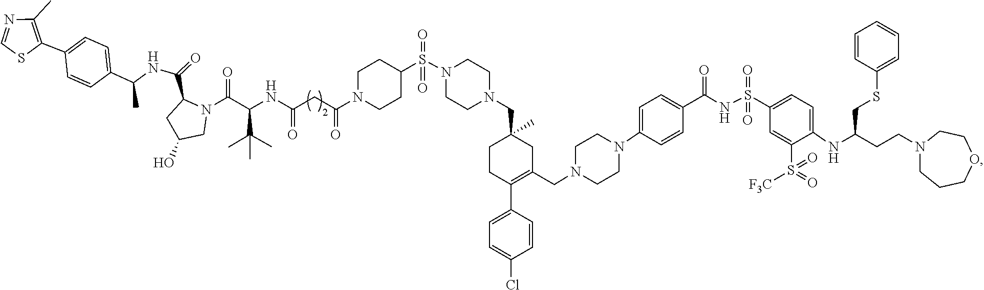 |
| 102 | 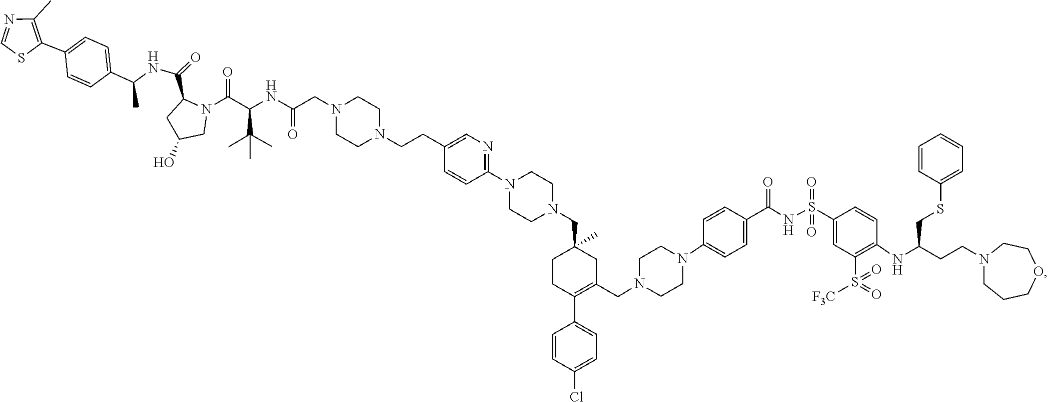 [SMILES: Cc1ncsc1-c1ccc([C@H](C)NC(=O)[C@@H]2C[C@@H](O)CN2C(=O)[C@@H](NC(=O)CN2CCN(CCc3ccc(N4CCN(C[C@]5(C)CCC(c6ccc(Cl)cc6)=C(CN6CCN(c7ccc(C(=O)NS(=O)(=O)c8ccc(N[C@H](CCN9CCCOCC9)CSc9ccccc9)c(S(=O)(=O)C(F)(F)F)c8)cc7)CC6)C5)CC4)nc3)CC2)C(C)(C)C)cc1] |
| 103 | 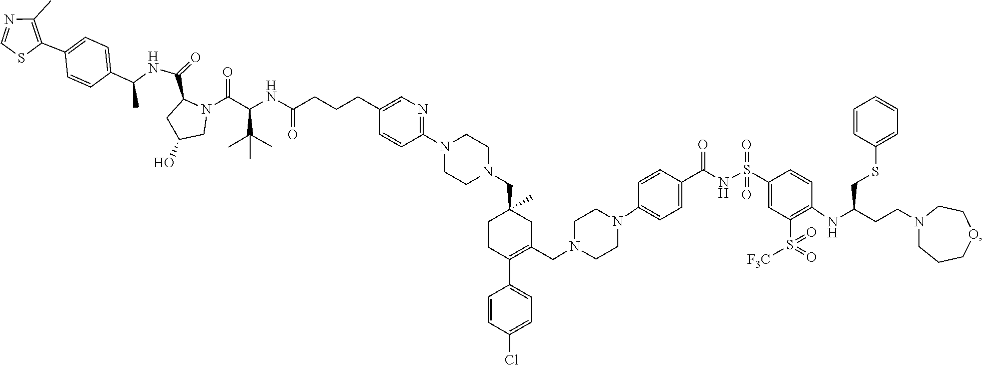 [SMILES: Cc1ncsc1-c1ccc([C@H](C)NC(=O)[C@@H]2C[C@@H](O)CN2C(=O)[C@@H](NC(=O)CCCc2ccc(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nc2)C(C)(C)C)cc1] |
| 104 | 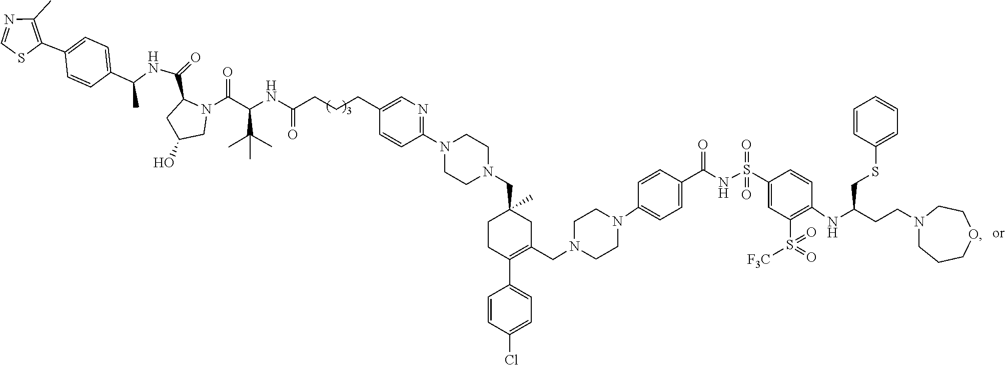 |
| 105 | 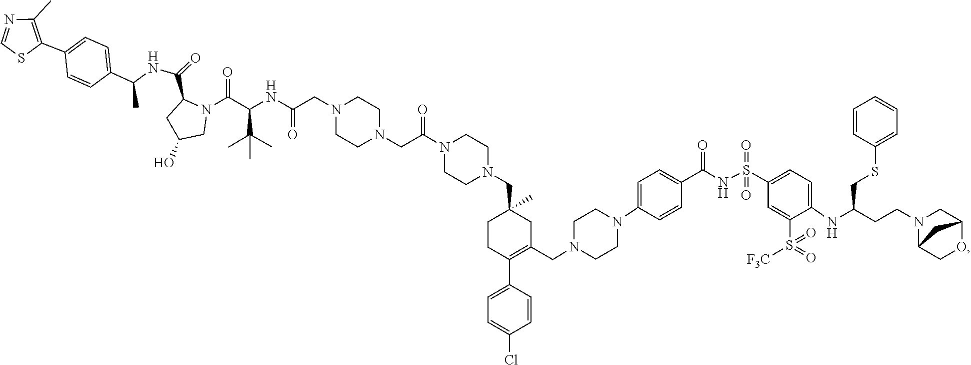 [SMILES: Cc1ncsc1-c1ccc([C@H](C)NC(=O)[C@@H]2C[C@@H](O)CN2C(=O)[C@@H](NC(=O)CN2CCN(CC(=O)N3CCN(C[C@]4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1] |

or a pharmaceutically acceptable salt, hydrate, prodrug thereof.

In another aspect, the compound of Formula (I) is:

| Com- | |
|---|---|
| pound | |
| # | Formula |
| 109 | 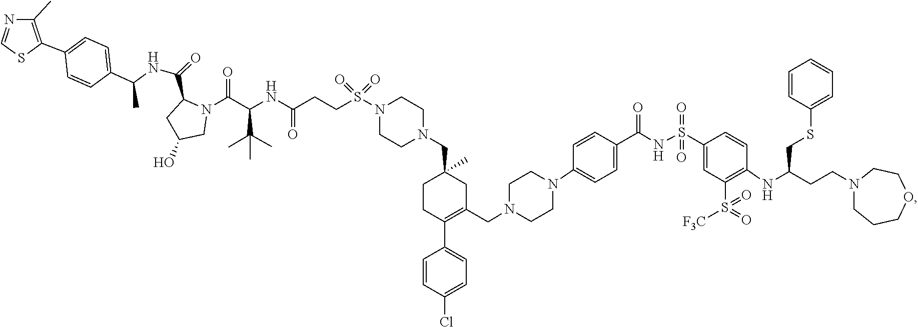 [SMILES: Cc1ncsc1-c1ccc([C@H](C)NC(=O)[C@@H]2C[C@@H](O)CN2C(=O)[C@@H](NC(=O)CCS(=O)(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)C(C)(C)C)cc1] |
| 110 | 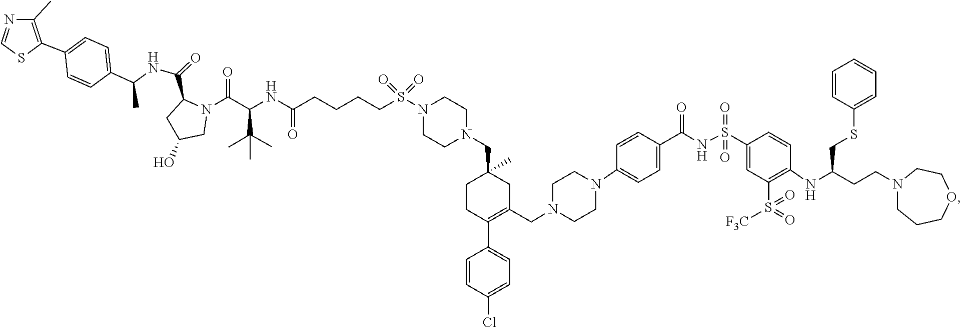 [SMILES: Cc1ncsc1-c1ccc([C@H](C)NC(=O)[C@@H]2C[C@@H](O)CN2C(=O)[C@@H](NC(=O)CCCCS(=O)(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)C(C)(C)C)cc1] |
| 111 | 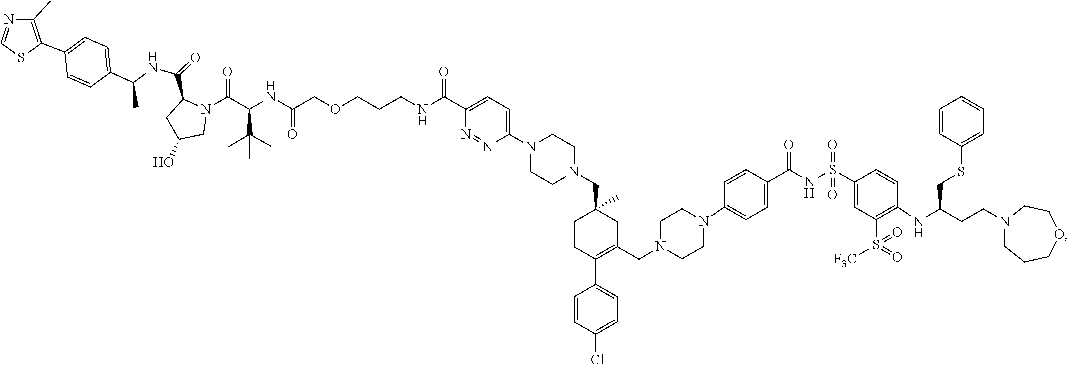 [SMILES: Cc1ncsc1-c1ccc([C@H](C)NC(=O)[C@@H]2C[C@@H](O)CN2C(=O)[C@@H](NC(=O)COCCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nn2)C(C)(C)C)cc1] |
| 112 | 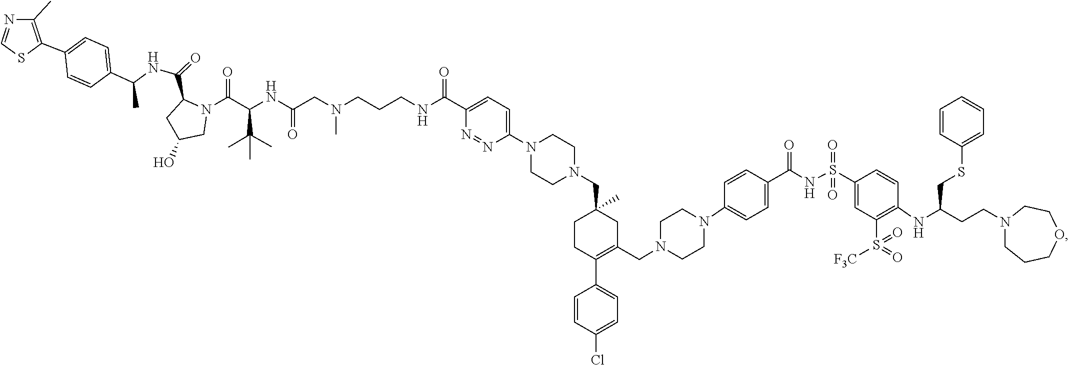 [SMILES: Cc1ncsc1-c1ccc([C@H](C)NC(=O)[C@@H]2C[C@@H](O)CN2C(=O)[C@@H](NC(=O)CN(C)CCCNC(=O)c2ccc(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nn2)C(C)(C)C)cc1] |
| 113 | 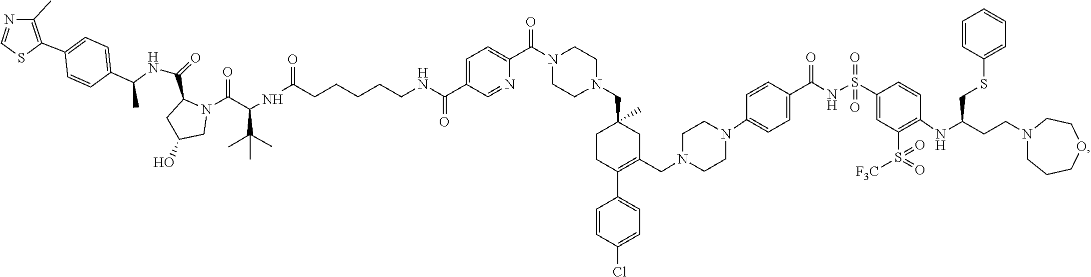 [SMILES: Cc1ncsc1-c1ccc([C@H](C)NC(=O)[C@@H]2C[C@@H](O)CN2C(=O)[C@@H](NC(=O)CCCCCNC(=O)c2ccc(C(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)nc2)C(C)(C)C)cc1] |
| 114 | 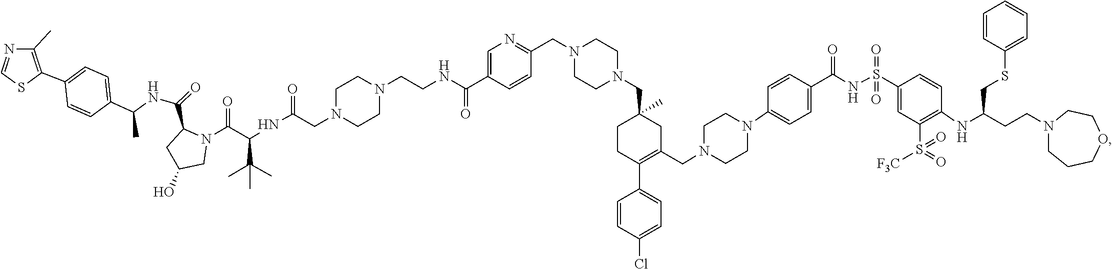 [SMILES: Cc1ncsc1-c1ccc([C@H](C)NC(=O)[C@@H]2C[C@@H](O)CN2C(=O)[C@@H](NC(=O)CN2CCN(CCNC(=O)c3ccc(CN4CCN(C[C@]5(C)CCC(c6ccc(Cl)cc6)=C(CN6CCN(c7ccc(C(=O)NS(=O)(=O)c8ccc(N[C@H](CCN9CCCOCC9)CSc9ccccc9)c(S(=O)(=O)C(F)(F)F)c8)cc7)CC6)C5)CC4)nc3)CC2)C(C)(C)C)cc1] |
| 115 | 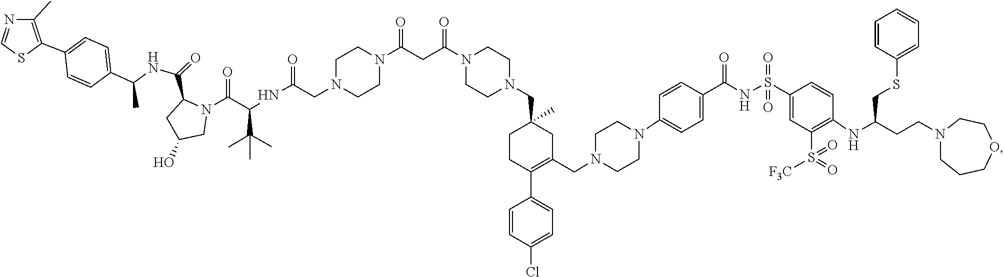 [SMILES: Cc1ncsc1-c1ccc([C@H](C)NC(=O)[C@@H]2C[C@@H](O)CN2C(=O)[C@@H](NC(=O)CN2CCN(C(=O)CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 116 | 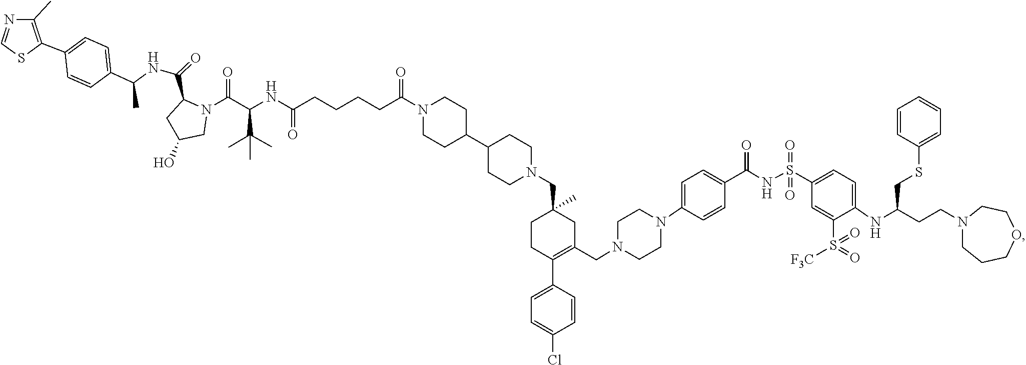 [SMILES: Cc1ncsc1-c1ccc([C@H](C)NC(=O)[C@@H]2C[C@@H](O)CN2C(=O)[C@@H](NC(=O)CCCCC(=O)N2CCC(C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 117 | 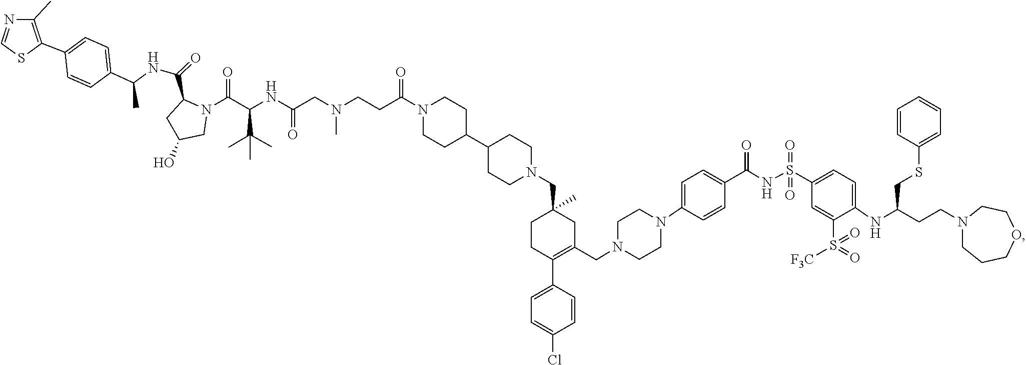 [SMILES: Cc1ncsc1-c1ccc([C@H](C)NC(=O)[C@@H]2C[C@@H](O)CN2C(=O)[C@@H](NC(=O)CN(C)CCC(=O)N2CCC(C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 118 | 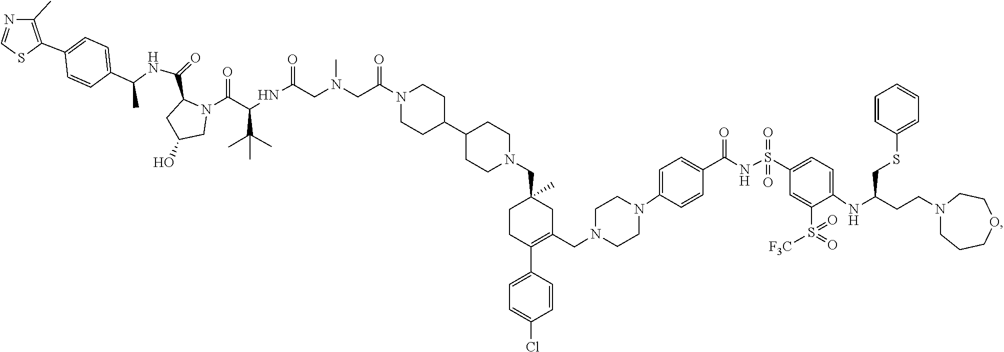 [SMILES: Cc1ncsc1-c1ccc([C@H](C)NC(=O)[C@@H]2C[C@@H](O)CN2C(=O)[C@@H](NC(=O)CN(C)CC(=O)N2CCC(C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |
| 119 | 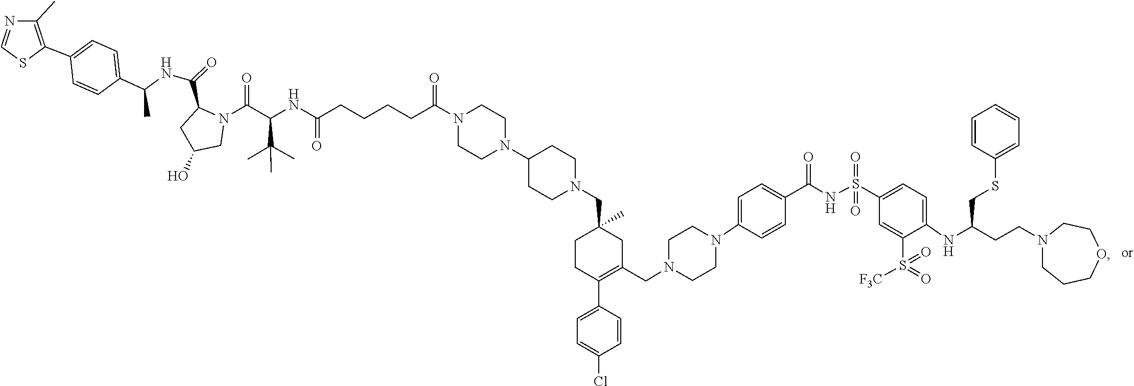 [SMILES: Cc1ncsc1-c1ccc([C@H](C)NC(=O)[C@@H]2C[C@@H](O)CN2C(=O)[C@@H](NC(=O)CCCCC(=O)N2CCN(C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1 ; "or"] |
| 120 | 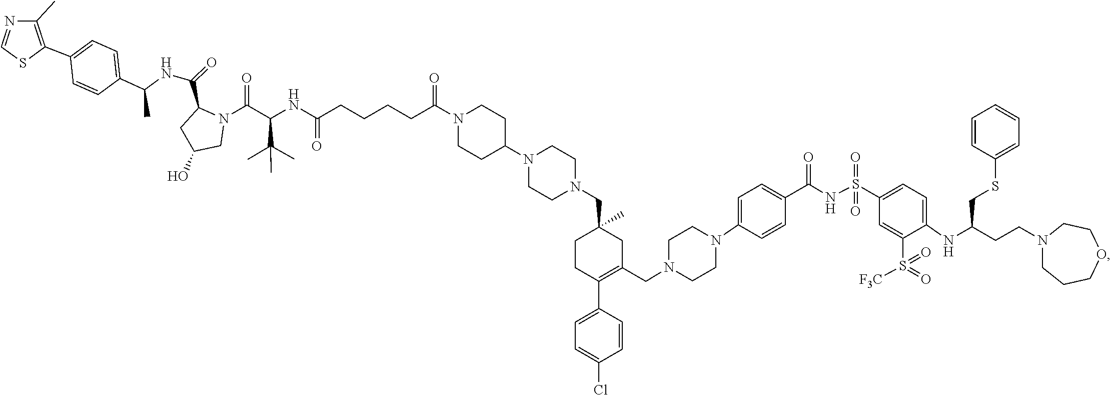 [SMILES: Cc1ncsc1-c1ccc([C@H](C)NC(=O)[C@@H]2C[C@@H](O)CN2C(=O)[C@@H](NC(=O)CCCCC(=O)N2CCC(N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1] |

or a pharmaceutically acceptable salt, hydrate, solvate, or prodrug thereof.

In certain embodiments, the compound is Compound 51, or a pharmaceutically acceptable salt, hydrate, solvate, or prodrug thereof (e.g., pharmaceutically acceptable salt thereof). In certain embodiments, the compound is not Compound 51, or a pharmaceutically acceptable salt, hydrate, solvate, or prodrug thereof (e.g., pharmaceutically acceptable salt thereof).

In another aspect, provided herein is a compound of Formula (II), or a pharmaceutically acceptable salt, hydrate, solvate, or prodrug thereof:

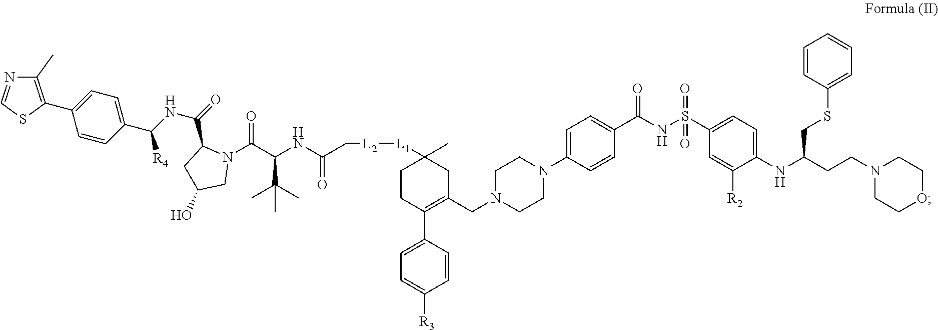

wherein:
R₂ is NO₂, SO₂CF₃, or SO₂CF₂Cl;
R₃ is Cl, F, CF₂H, CFH₂, or CF₃;
R₄ is H or CH₃;
L₁ is

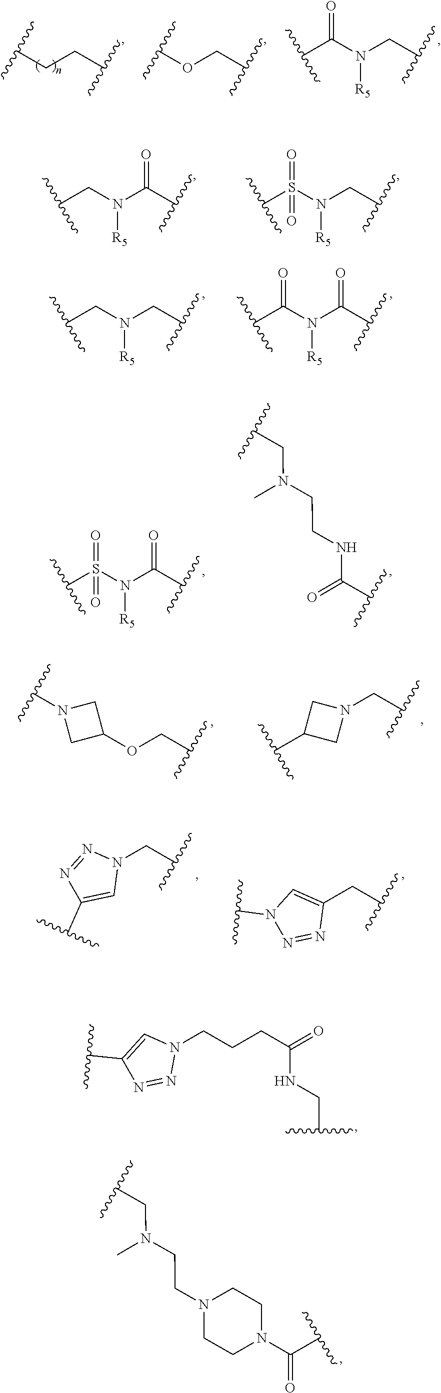

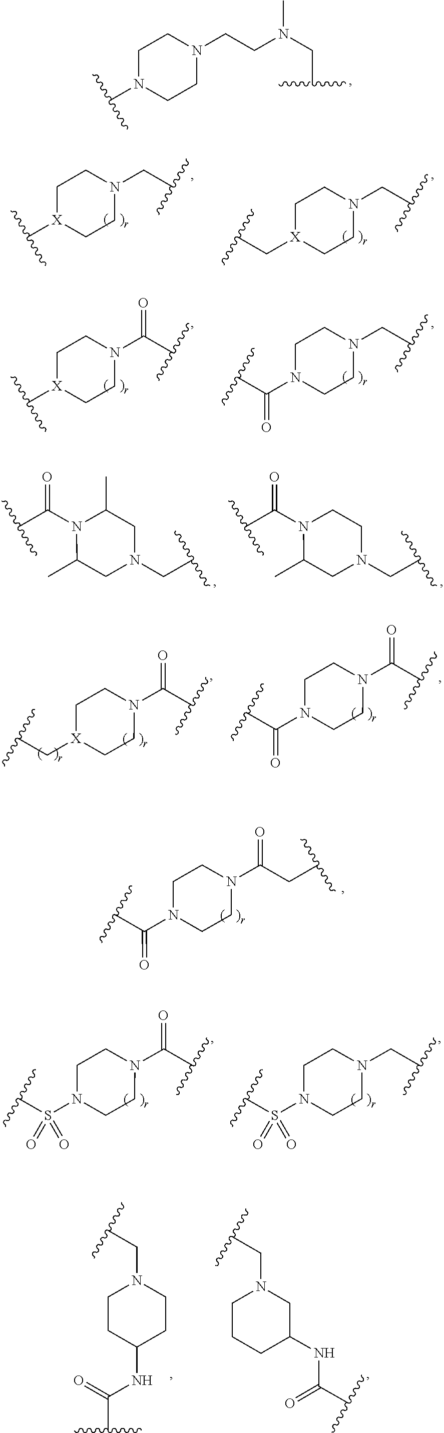

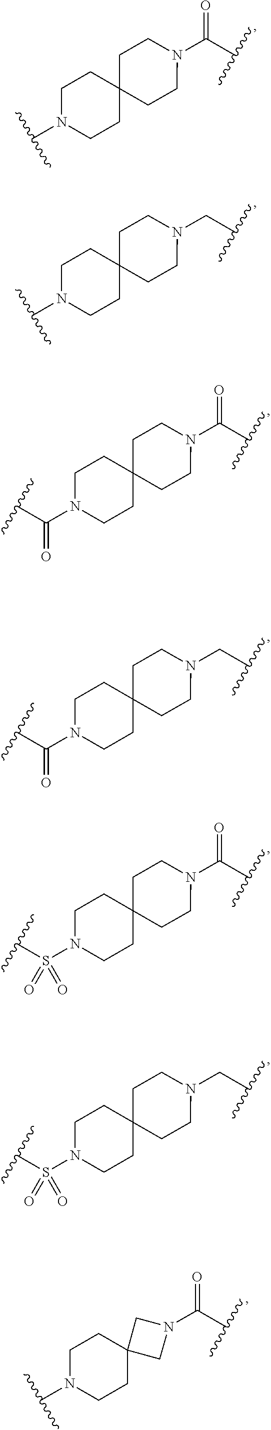

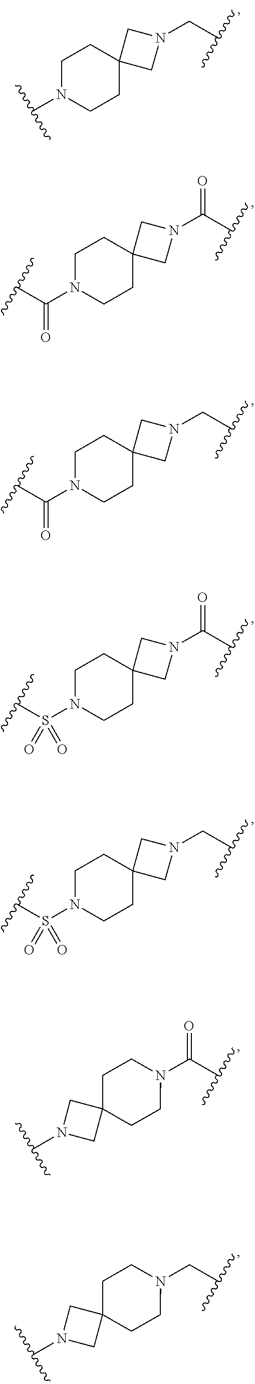

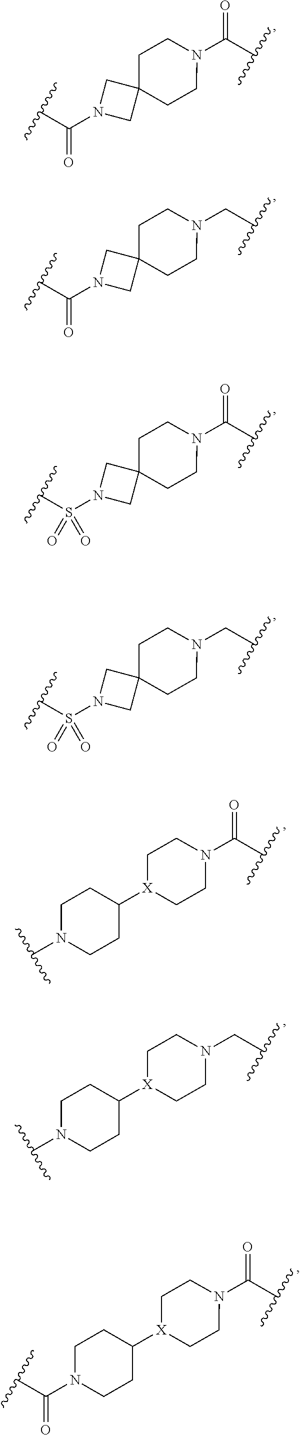

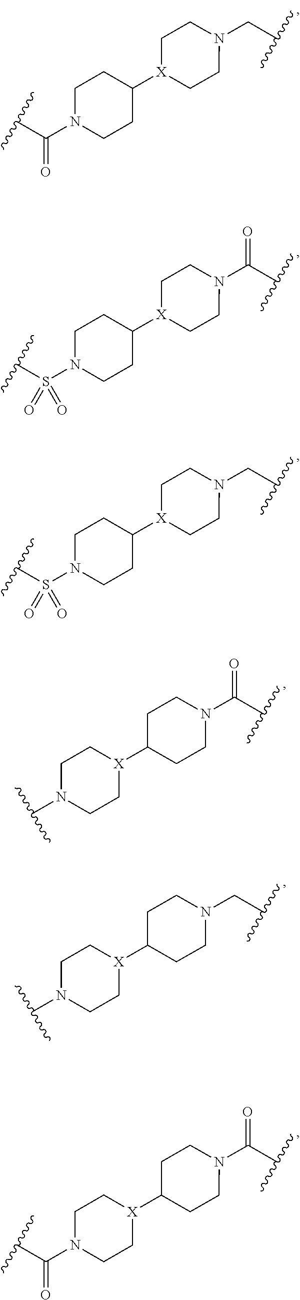

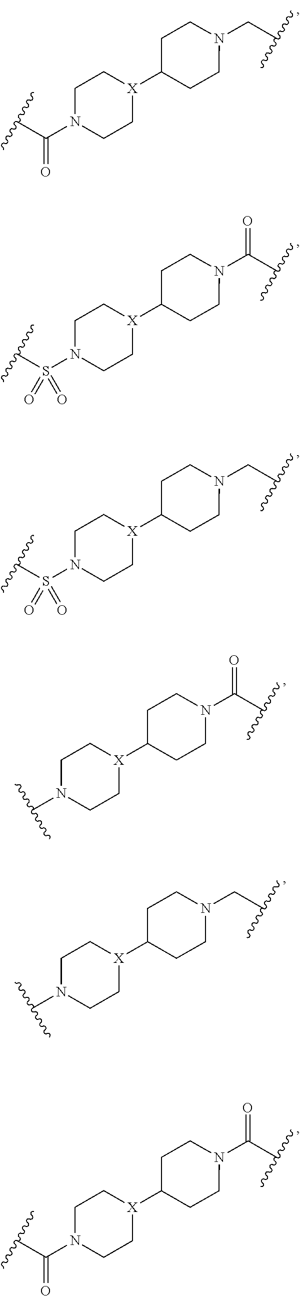

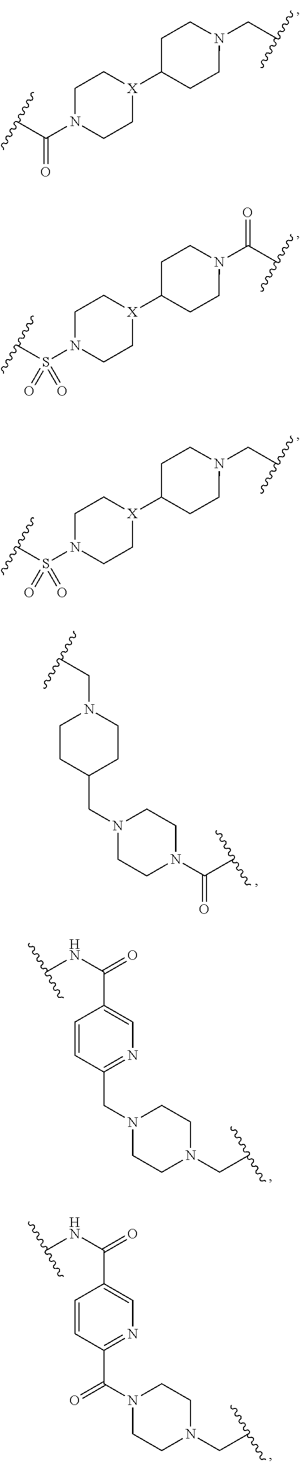

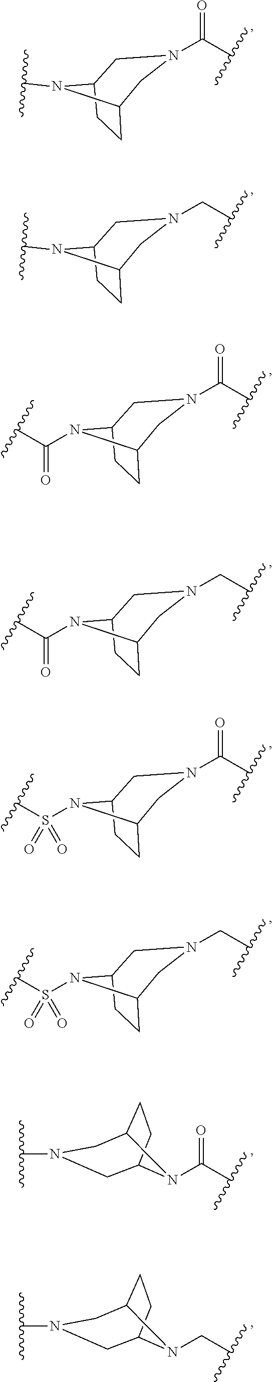

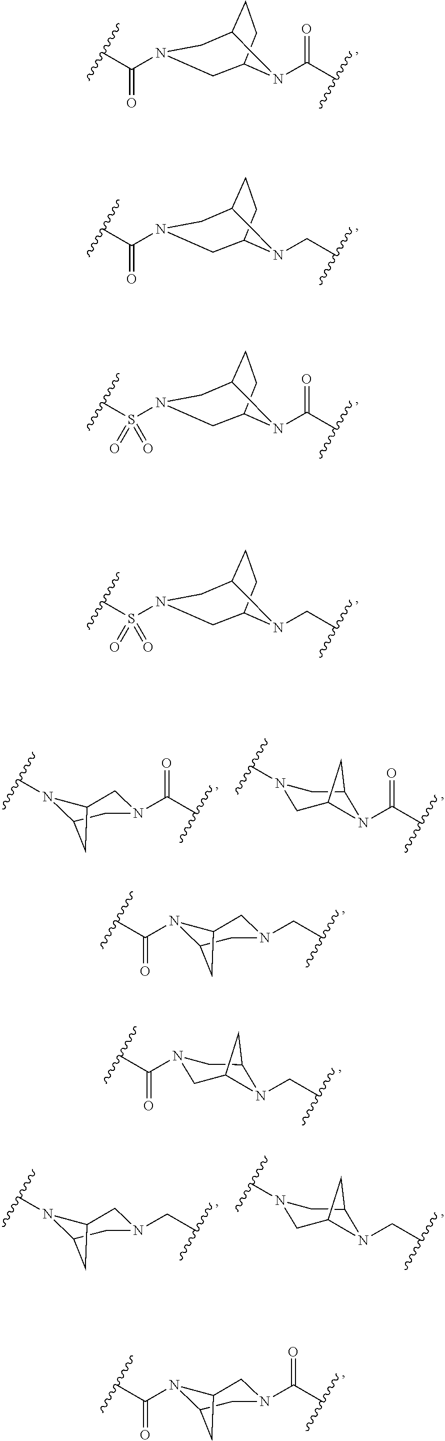

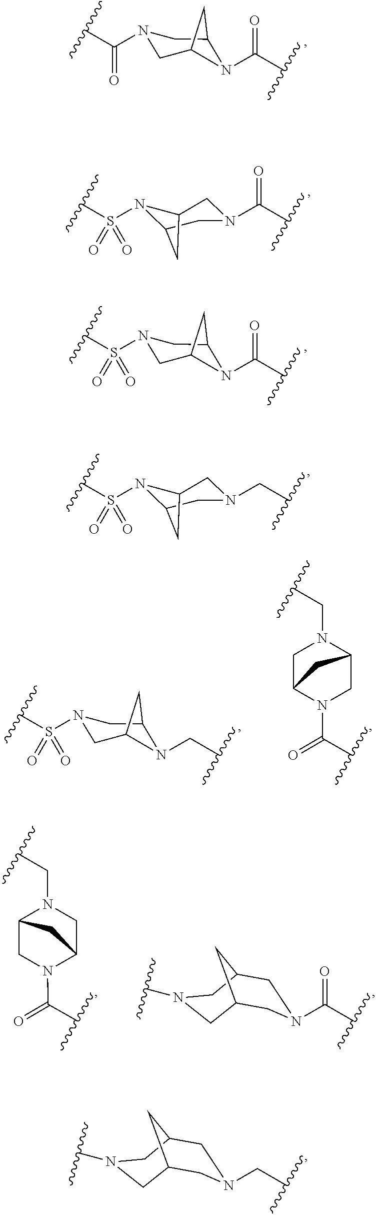

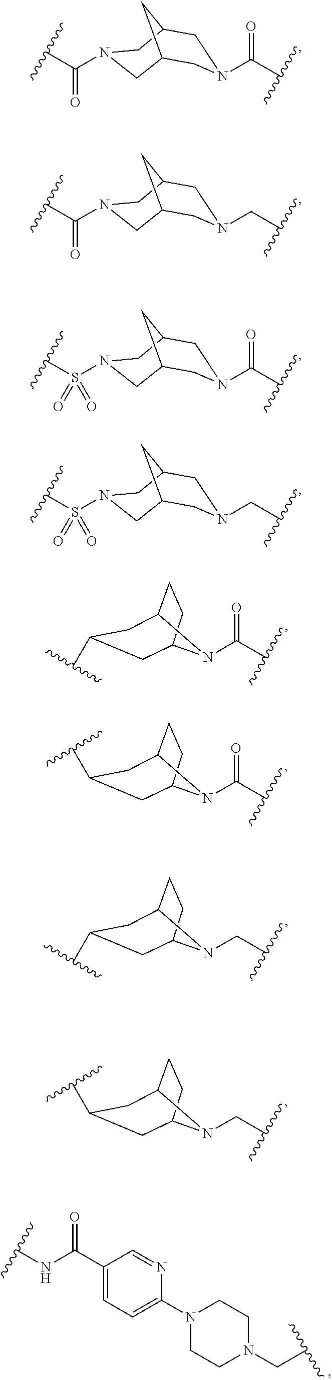

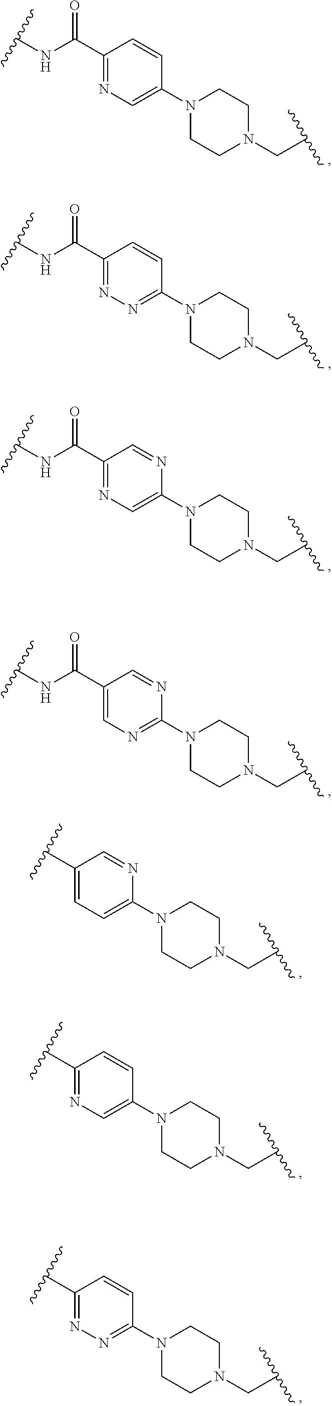

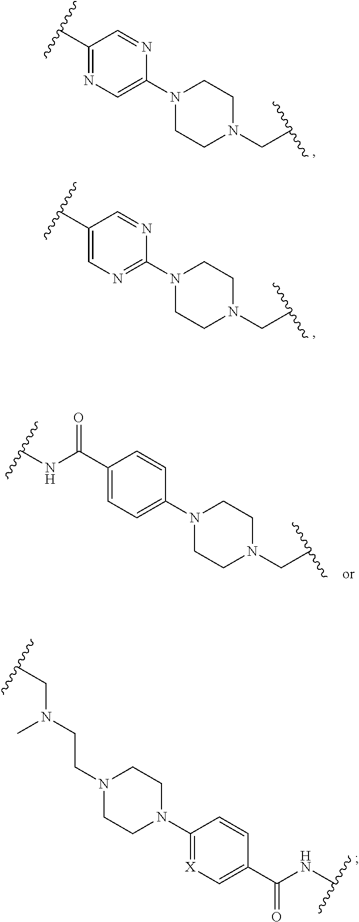

L₂ is a bond,

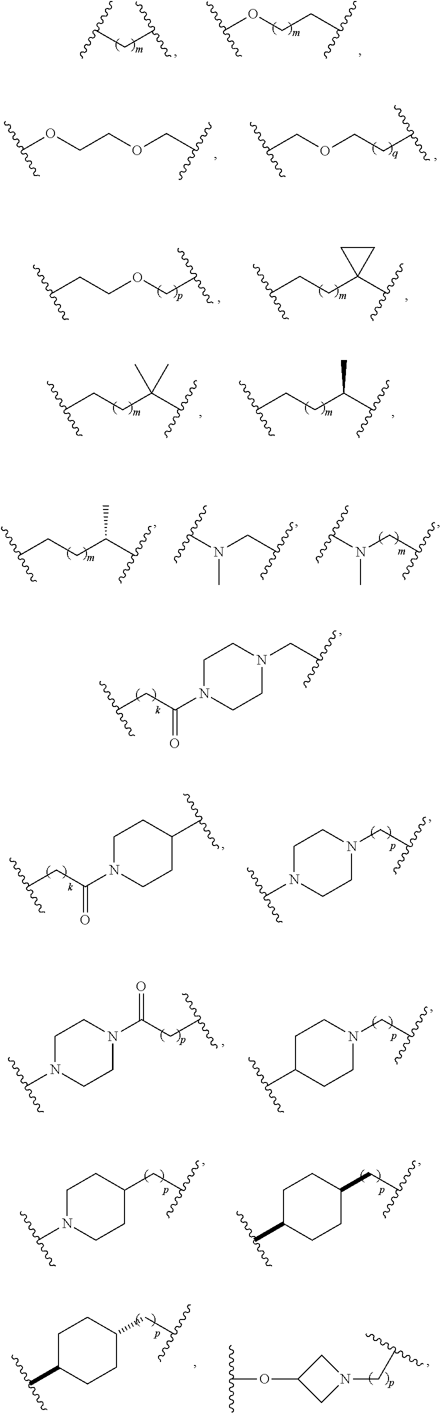

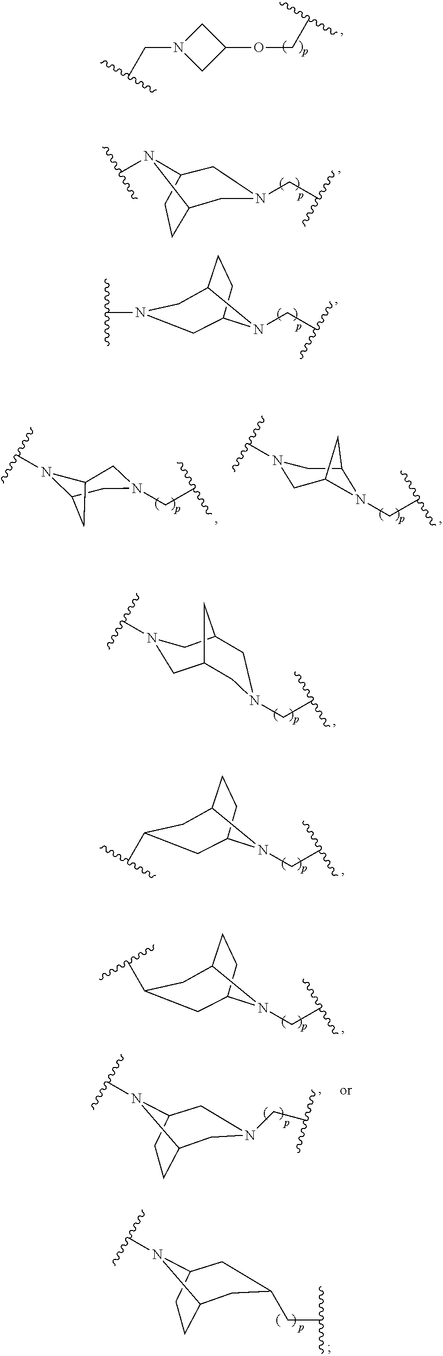

each X is independently CH or N
each k is independently 0, 1, 2, 3, 4, 5, or 6;
each m is independently 2, 3, 4, 5, 6, or 7;
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl; provided that when R₂ is SO₂CF₃ and L₁ is

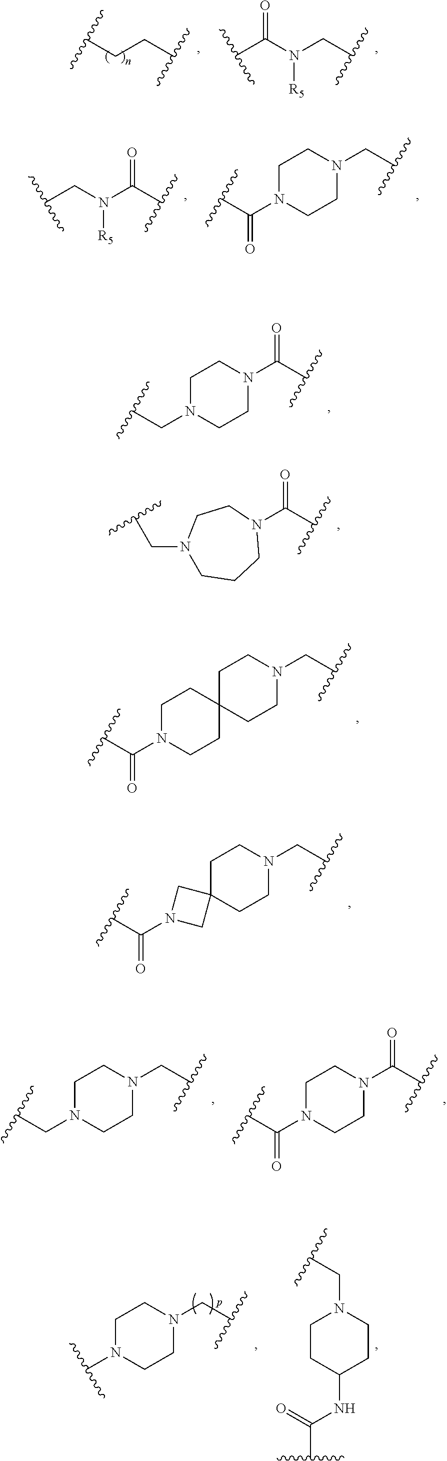

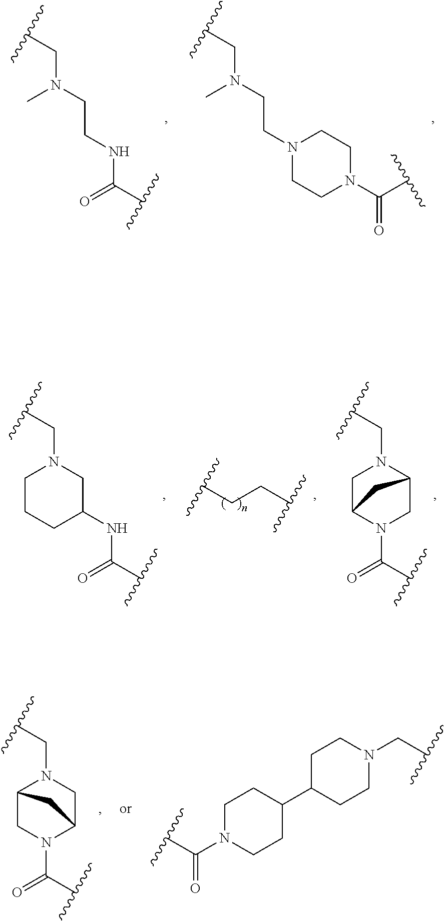

then L₂ is not a bond,

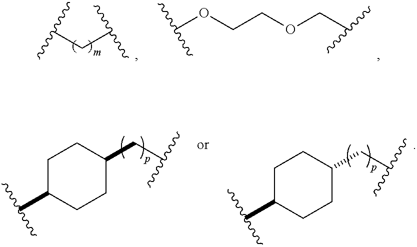

In another aspect, the compound of Formula (II), or a pharmaceutically acceptable salt, hydrate, solvate, or prodrug thereof, is of Formula (II-A):

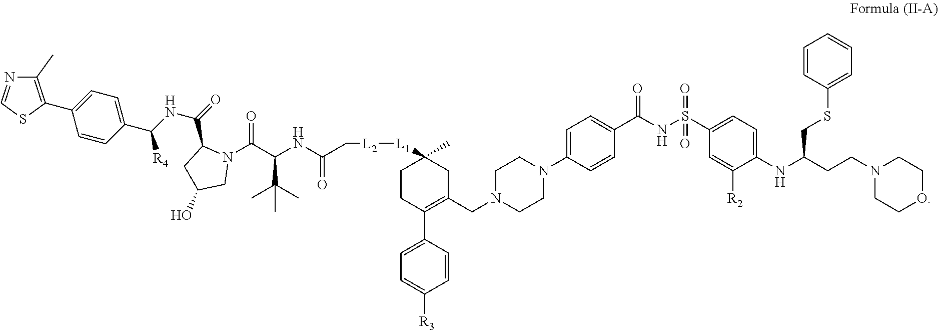

In another aspect, the compound of Formula (II), or a pharmaceutically acceptable salt, hydrate, solvate, or prodrug thereof, is of Formula (II-B):

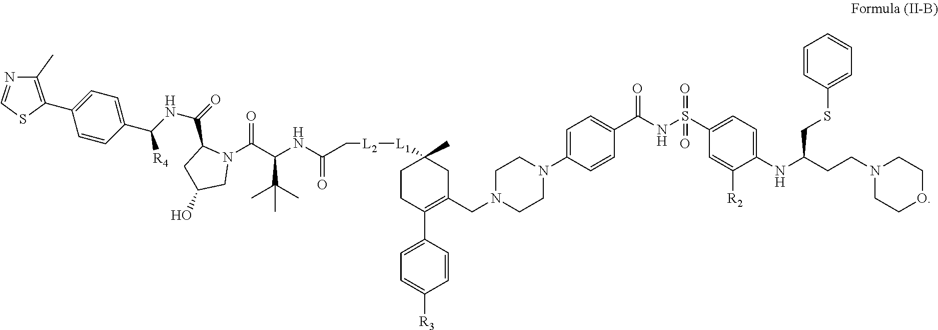

In one aspect, R₂ is NO₂, SO₂CF₃, or SO₂CF₂Cl. In one aspect, R₂ is NO₂ or SO₂CF₃. In one aspect, R₂ is NO₂ or SO₂CF₂Cl. In one aspect, R₂ is SO₂CF₃ or SO₂CF₂Cl. In one aspect, R₂ is NO₂. In another aspect, R₂ is SO₂CF₃. In one aspect, R₂ is SO₂CF₂Cl.

In another aspect, the compound of Formula (II), or a pharmaceutically acceptable salt, hydrate, solvate, or prodrug thereof, is of Formula (II-C):

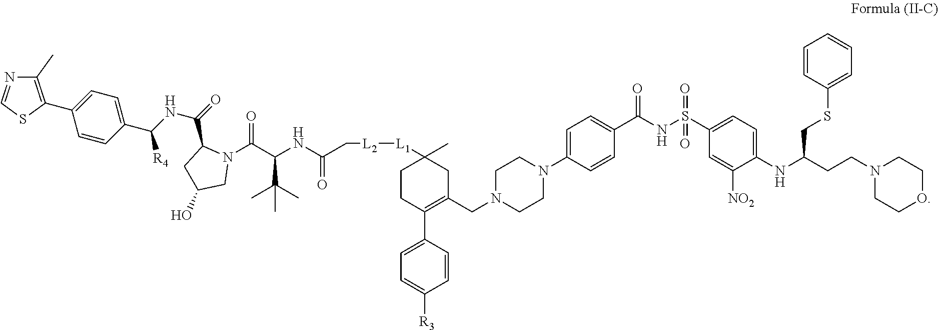

In another aspect, the compound of Formula (II), or a pharmaceutically acceptable salt, hydrate, solvate, or prodrug thereof, is of Formula (II-D):

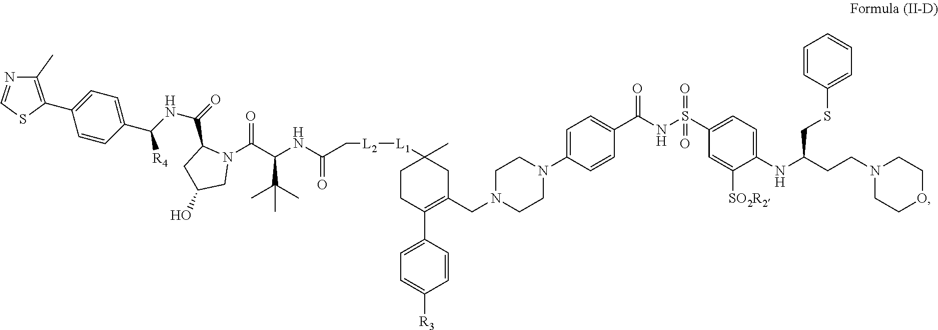

wherein R_{2′} is CF₃, or CF₂Cl.

In one aspect, R₃ is Cl, F, CFH₂, CF₂H, or CF₃. In some embodiments, R₃ is F, CFH₂, CF₂H, or CF₃. In certain embodiments, R₃ is CFH₂, CF₂H, or CF₃. In some embodiments, R₃ is Cl, F, or CF₃. In certain embodiments, R₃ is Cl or F. In some embodiments, R₃ is CF₃ or F. In certain embodiments, R₃ is Cl. In some embodiments, R₃ is F. In certain embodiments, R₃ is CF₃.

In some embodiments, R₃ is CF₂H. In certain embodiments, R₃ is CFH₂. In one aspect, R₄ is H or CH₃. In some embodiments, R₄ is H. In certain embodiments, R₄ is CH₃.

In one aspect, R₅ is independently H, optionally substituted alkyl, or optionally substituted cycloalkyl. In some embodiments, R₅ is independently H or optionally substituted cycloalkyl. In certain embodiments, R₅ is independently H or optionally substituted alkyl. In some embodiments, R₅ is independently optionally substituted alkyl or optionally substituted cycloalkyl. In certain embodiments, R₅ is independently H, unsubstituted alkyl, or unsubstituted cycloalkyl. In some embodiments, R₅ is independently H or unsubstituted cycloalkyl. In certain embodiments, R₅ is independently H or unsubstituted alkyl. In some embodiments, R₅ is independently unsubstituted alkyl or unsubstituted cycloalkyl.

In certain embodiments, R₅ is independently H, optionally substituted C₁-C₁₀ alkyl, or optionally substituted C₃-C₁₀ cycloalkyl. In some embodiments, R₅ is independently H or optionally substituted C₃-C₁₀ cycloalkyl. In certain embodiments, R₅ is independently H or optionally substituted C₁-C₁₀ alkyl. In some embodiments, R₅ is independently optionally substituted C₁-C₁₀ alkyl or optionally substituted C₃-C₁₀ cycloalkyl. In certain embodiments, R₅ is independently H, unsubstituted C₁-C₁₀ alkyl, or unsubstituted C₃-C₁₀ cycloalkyl. In some embodiments, R₅ is independently H or unsubstituted C₃-C₁₀ cycloalkyl. In certain embodiments, R₅ is independently H or unsubstituted C₁-C₁₀ alkyl. In some embodiments, R₅ is independently unsubstituted C₁-C₁₀ alkyl or unsubstituted C₃-C₁₀ cycloalkyl.

In certain embodiments, R₅ is independently H, optionally substituted C₁-C₆ alkyl, or optionally substituted C₃-C₇ cycloalkyl. In some embodiments, R₅ is independently H or optionally substituted C₃-C₇ cycloalkyl. In certain embodiments, R₅ is independently H or optionally substituted C₁-C₆ alkyl. In some embodiments, R₅ is independently optionally substituted C₁-C₆ alkyl or optionally substituted C₃-C₇ cycloalkyl. In certain embodiments, R₅ is independently H, unsubstituted C₁-C₆ alkyl, or unsubstituted C₃-C₇ cycloalkyl. In some embodiments, R₅ is independently H or unsubstituted C₃-C₇ cycloalkyl. In certain embodiments, R₅ is independently H or unsubstituted C₁-C₆ alkyl. In some embodiments, R₅ is independently unsubstituted C₁-C₆ alkyl or unsubstituted C₃-C₇ cycloalkyl. In certain embodiments, R₅ is H, methyl, ethyl, propyl, butyl, pentyl, hexyl, cyclopropyl, cyclobutyl, cyclopentyl, cyclohexyl, or cycloheptyl. In some embodiments, R₅ is H, CH₃, or CH₂CH₃. In some embodiments, R₅ is H or CH₃. In some embodiments, R₅ is CH₃ or CH₂CH₃.

In certain embodiments, R₅ is H. In some embodiments, R₅ is optionally substituted C₁-C₆ alkyl. In certain embodiments, R₅ is unsubstituted C₁-C₆ alkyl. In some embodiments, R₅ is CH₃. In certain embodiments, R₅ is CH₂CH₃. In some embodiments, R₅ is optionally substituted C₃-C₇ cycloalkyl. In certain embodiments, R₅ is unsubstituted C₃-C₇ cycloalkyl.

In some embodiments, L₁ is

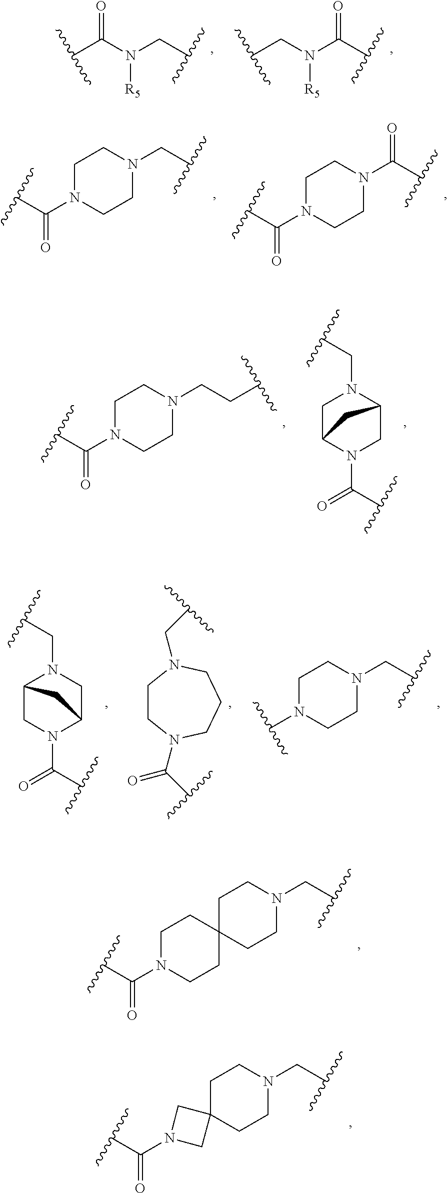

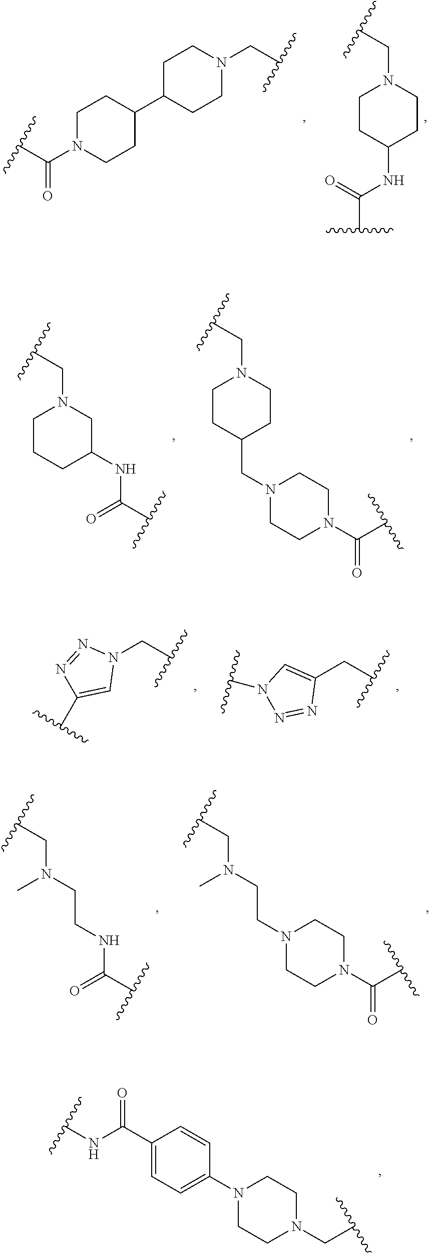

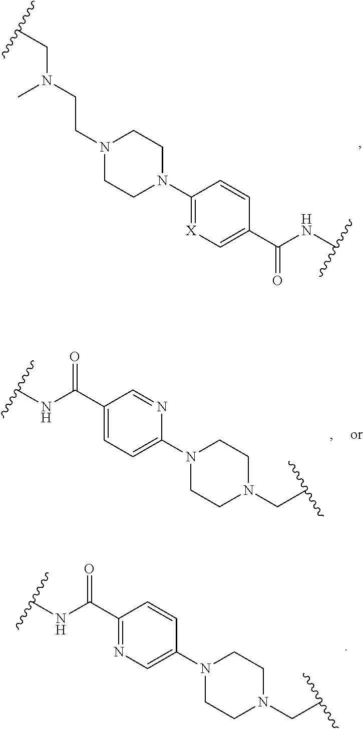

In some embodiments, L₁ is

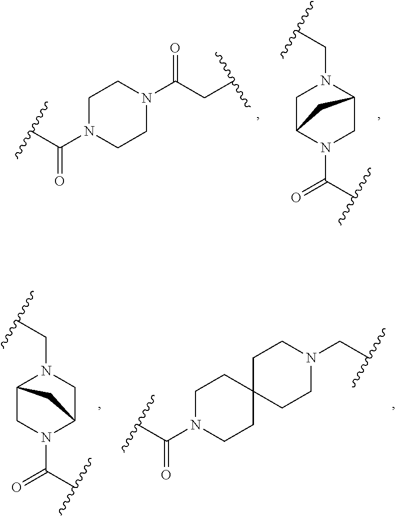 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2]

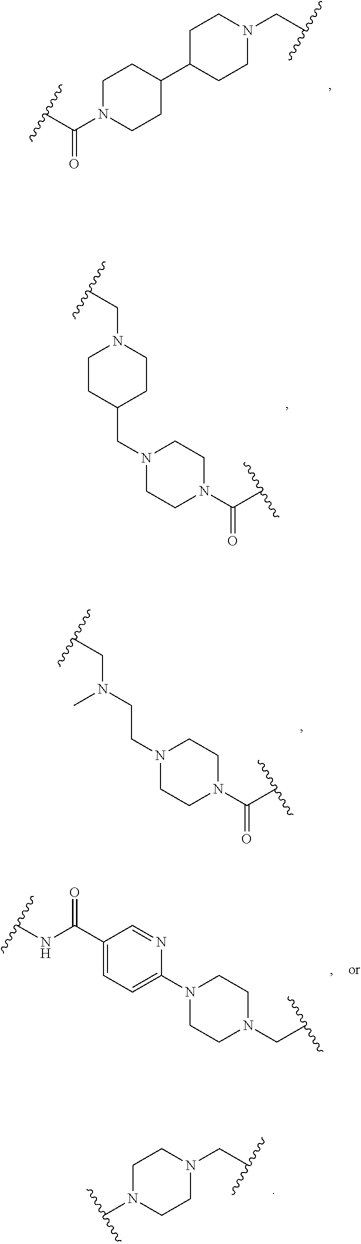 [SMILES: *CN1CCC(C2CCN(C(*)=O)CC2)CC1 ; *CN1CCC(CN2CCN(C(*)=O)CC2)CC1 ; *CN(C)CCN1CCN(C(*)=O)CC1 ; *CN1CCN(c2ccc(C(=O)N*)cn2)CC1 ; *CN1CCN(*)CC1]

In some embodiments, L₁ is

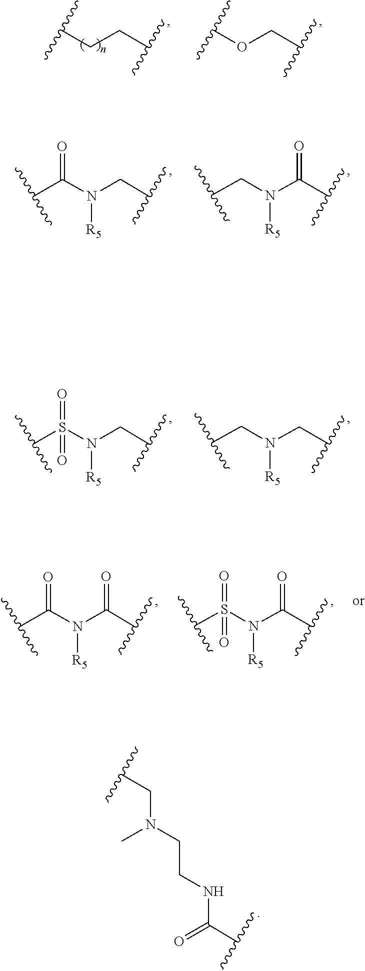

In some embodiments, L₁ is

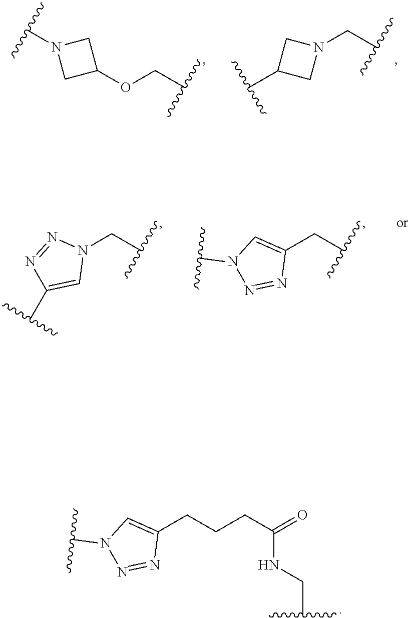 [SMILES: *COC1CN(*)C1 ; *CN1CC(*)C1 ; *Cn1cc(*)nn1 ; *Cc1cn(*)nn1 ; *CNC(=O)CCCc1cn(*)nn1]

In some embodiments, In some embodiments, L₁ is

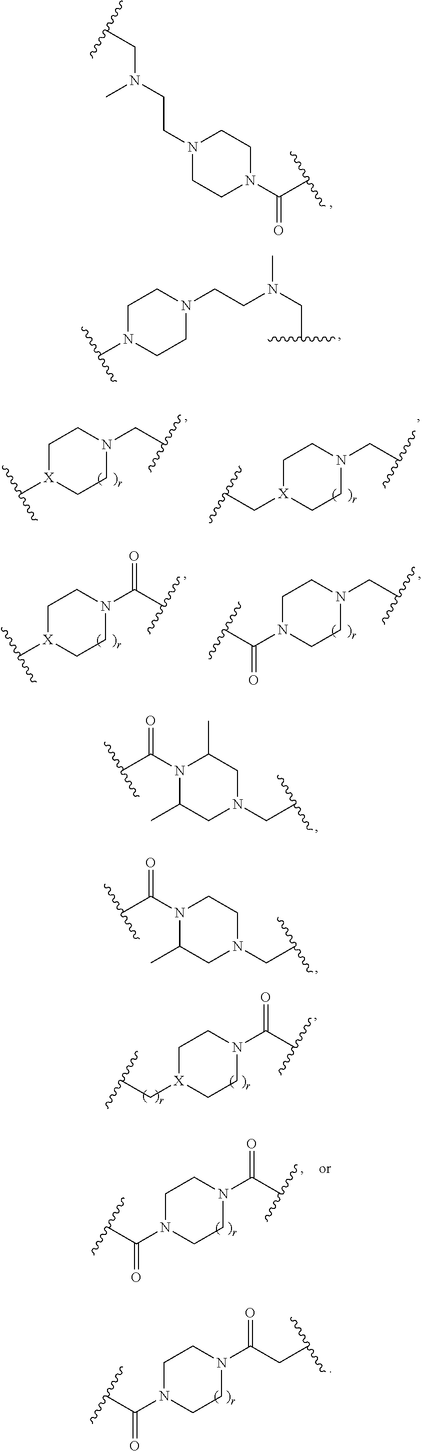

In some embodiments, L₁ is

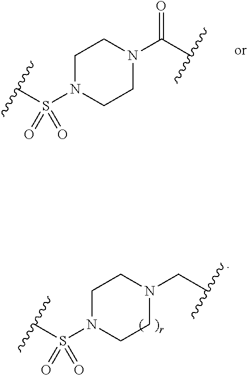

In some embodiments, L₁ is

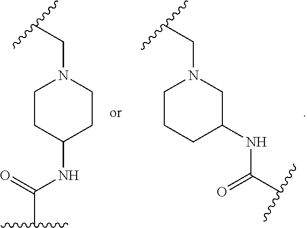 [SMILES: *CN1CCC(NC(*)=O)CC1 ; *CN1CCCC(NC(*)=O)C1]

In some embodiments, L₁ is

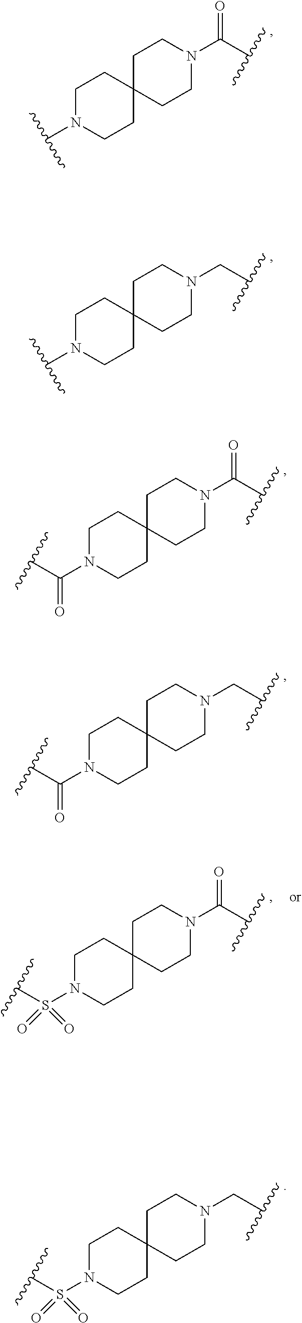 [SMILES: *C(=O)N1CCC2(CCN(*)CC2)CC1 ; *CN1CCC2(CCN(*)CC2)CC1 ; *C(=O)N1CCC2(CC1)CCN(C(*)=O)CC2 ; *CN1CCC2(CC1)CCN(C(*)=O)CC2 ; *C(=O)N1CCC2(CC1)CCN(S(*)(=O)=O)CC2 ; *CN1CCC2(CC1)CCN(S(*)(=O)=O)CC2]

In some embodiments, L₁ is

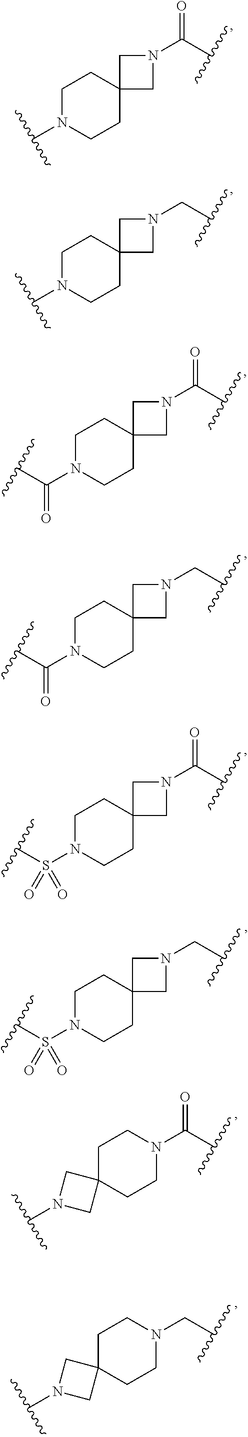

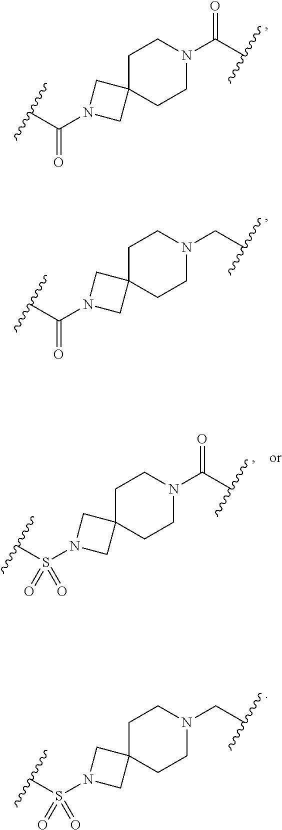 [SMILES: *C(=O)N1CCC2(CC1)CN(C(*)=O)C2 ; *CN1CCC2(CC1)CN(C(*)=O)C2 ; *C(=O)N1CCC2(CC1)CN(S(*)(=O)=O)C2 ; *CN1CCC2(CC1)CN(S(*)(=O)=O)C2]

In some embodiments, L₁ is

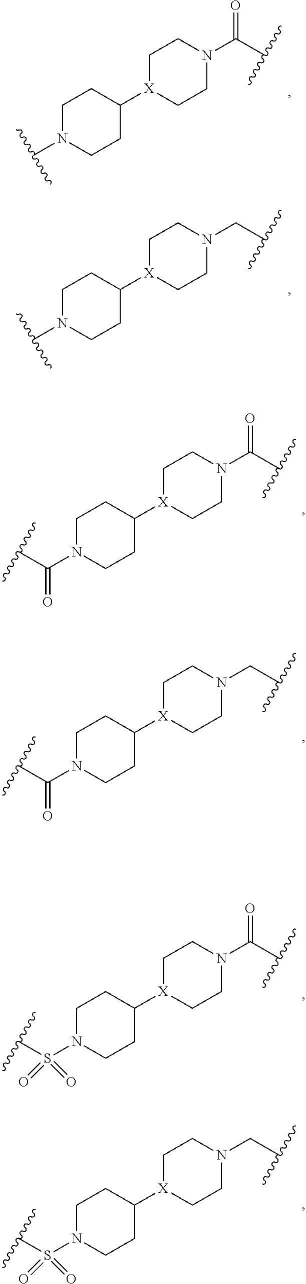

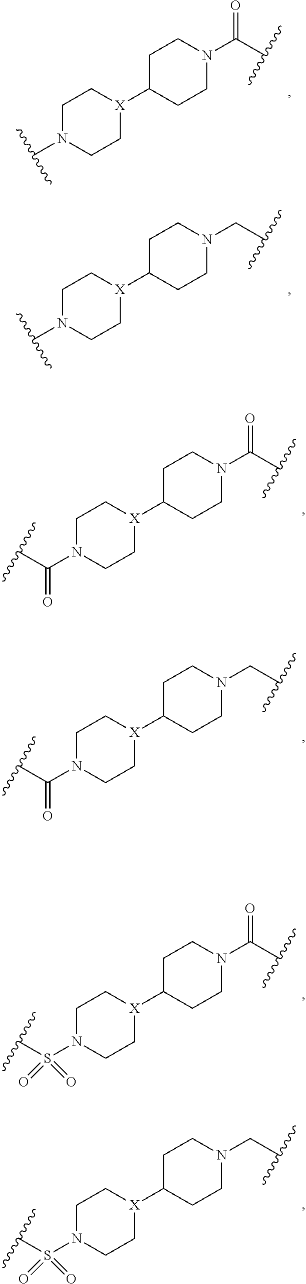

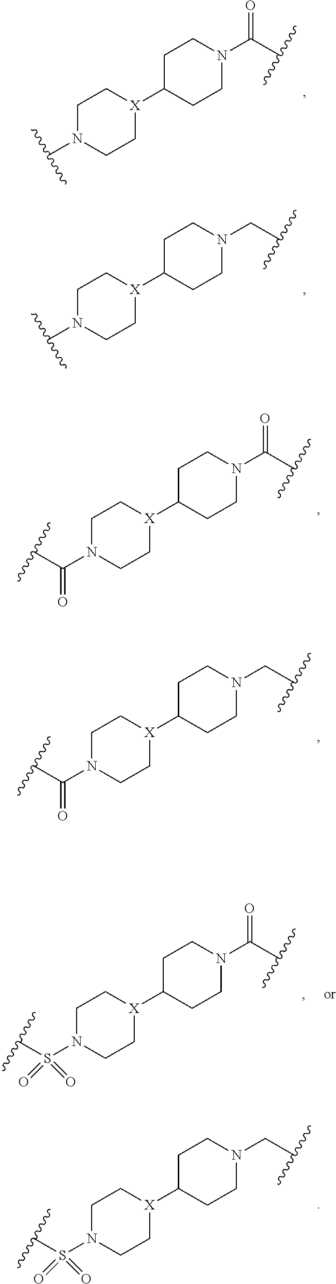

In some embodiments, L₁ is

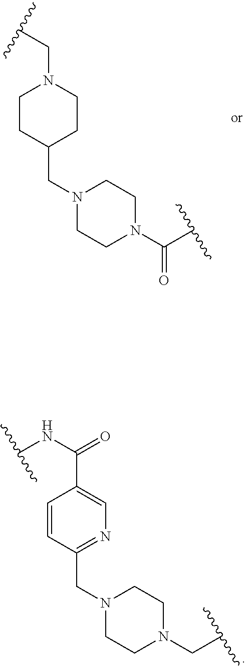 [SMILES: *CN1CCC(CN2CCN(C(*)=O)CC2)CC1 ; *CN1CCN(Cc2ccc(C(=O)N*)cn2)CC1]

In certain embodiments, L₁ is

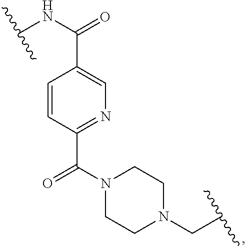 [SMILES: *CN1CCN(C(=O)c2ccc(C(=O)N*)cn2)CC1]

In some embodiments, L₁ is

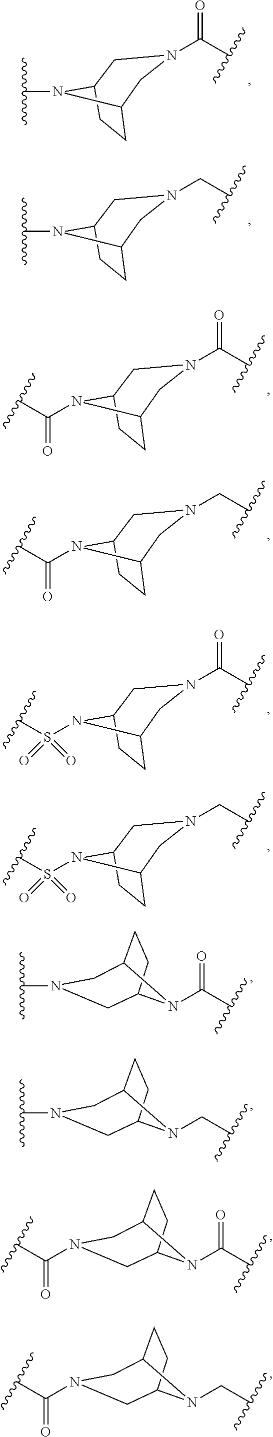

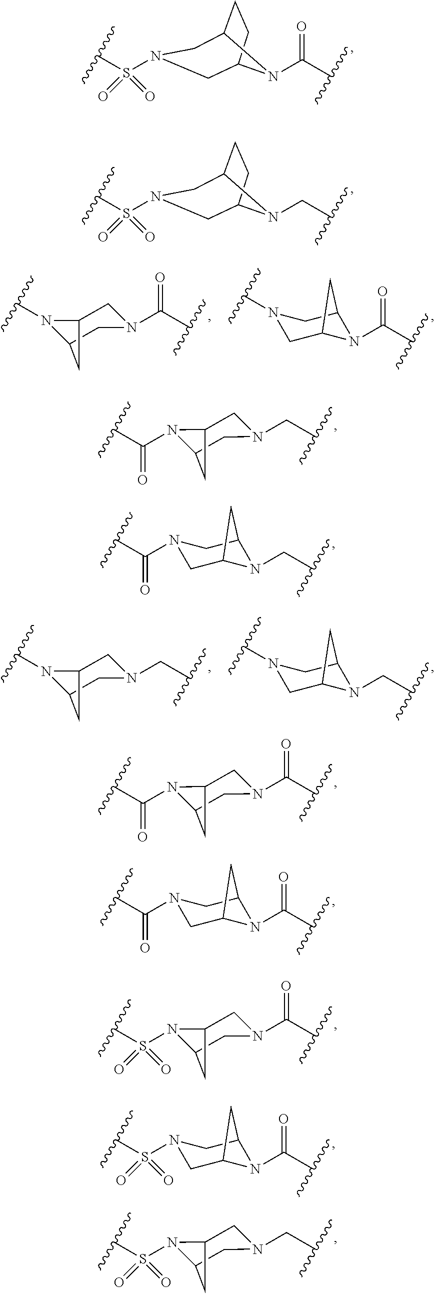

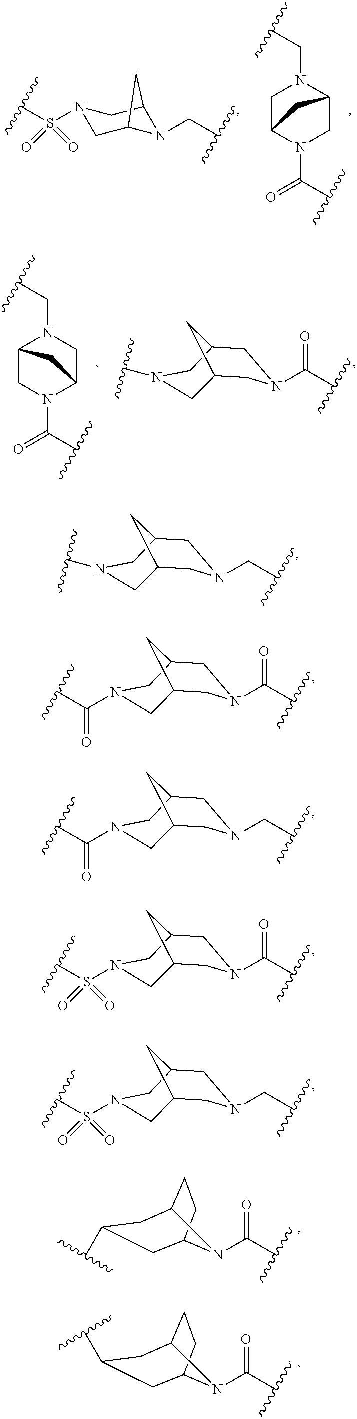

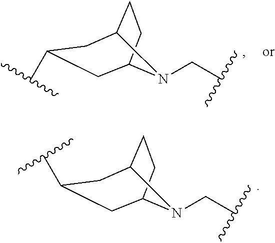 [SMILES: *CN1C2CCC1CC(*)C2 ; *CN1C2CCC1CC(*)C2]

In some embodiments, L₁ is

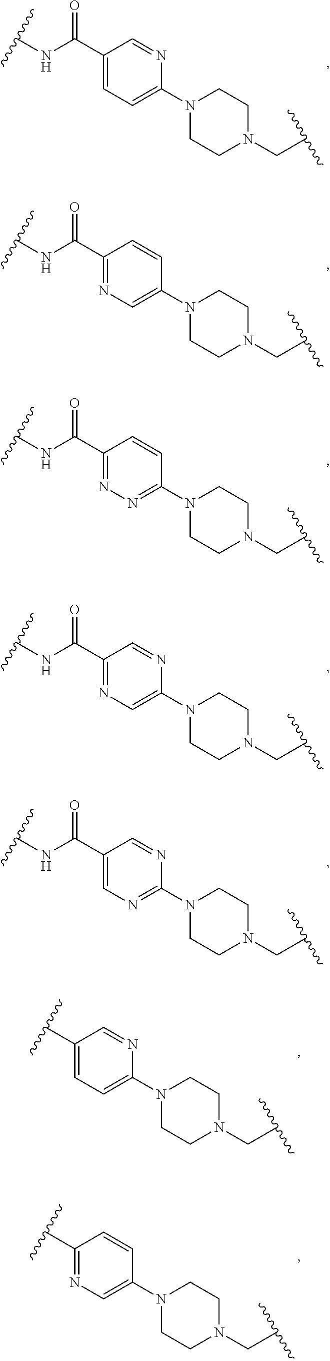

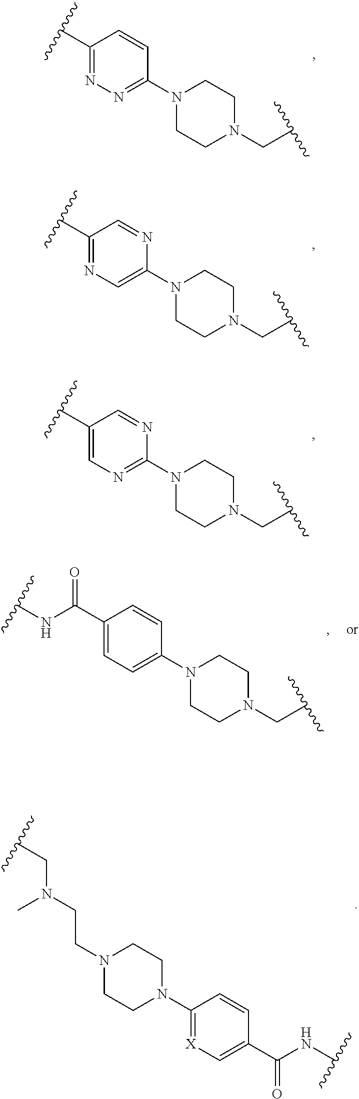

In some embodiments, L₁ is not

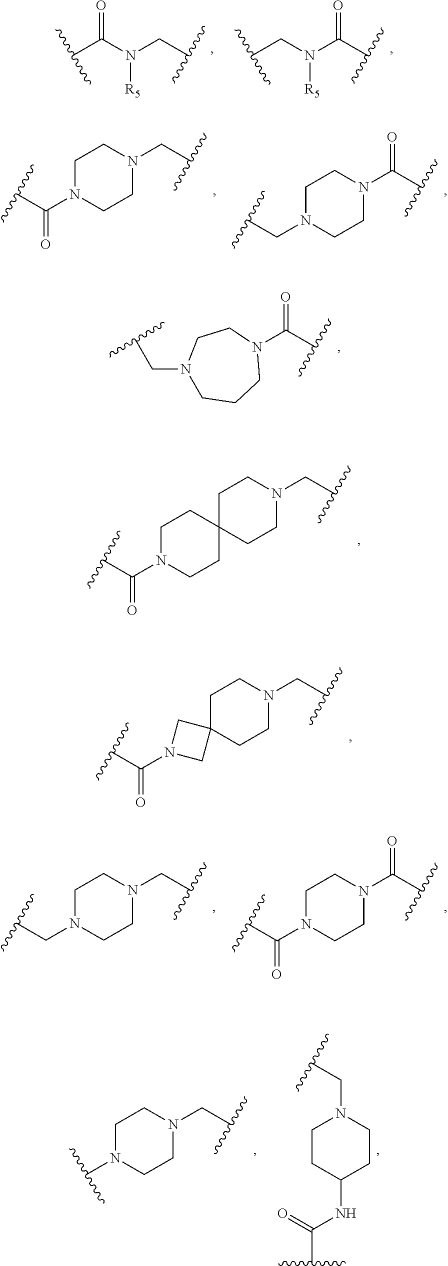

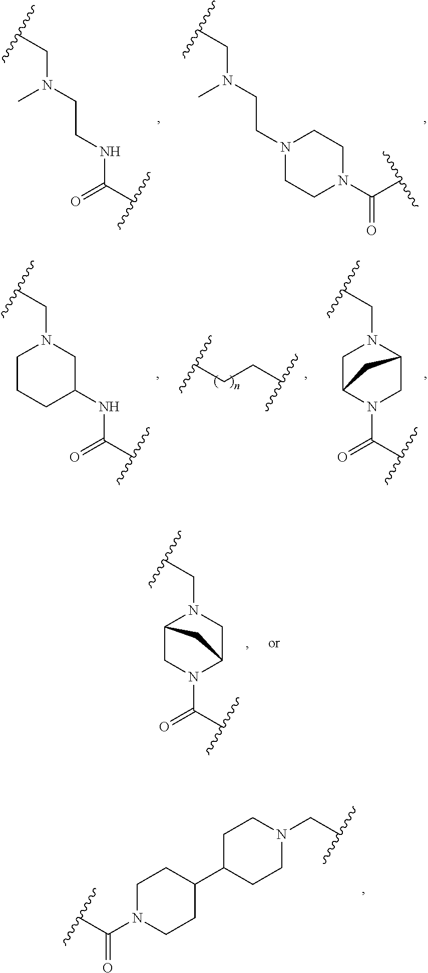

In some embodiments, L₁ is

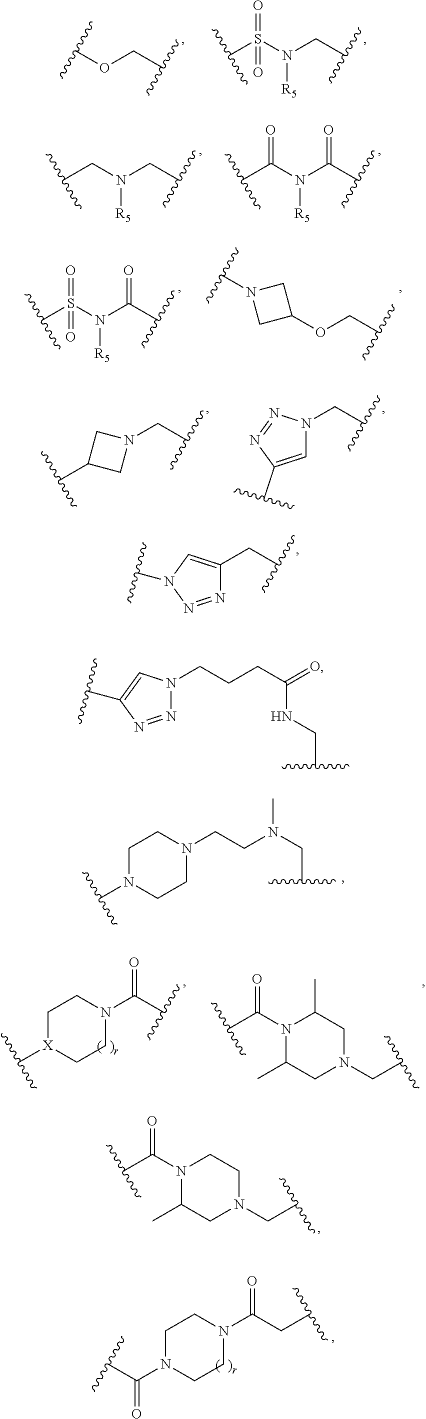

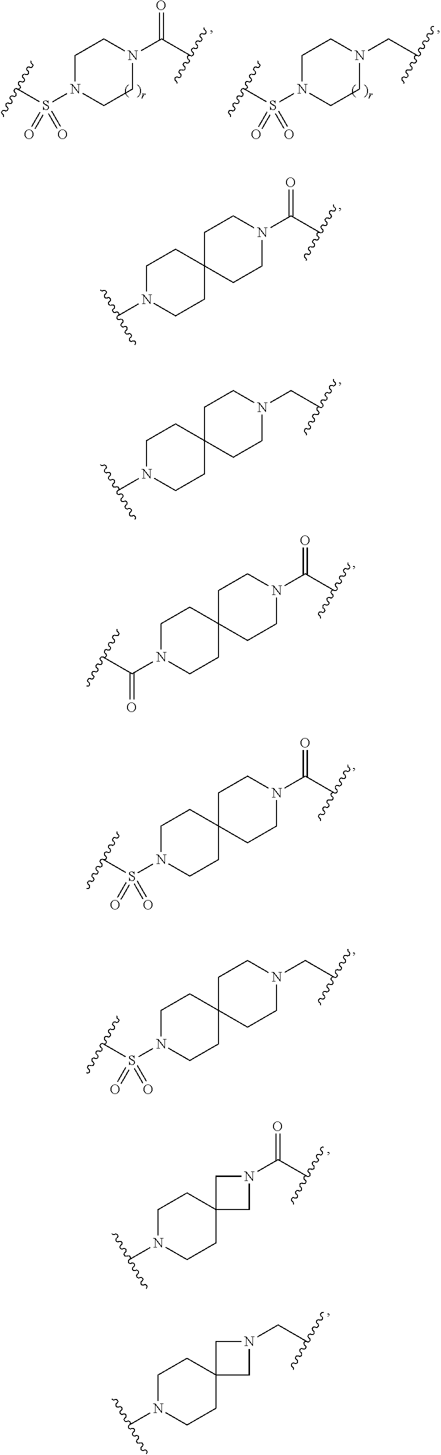

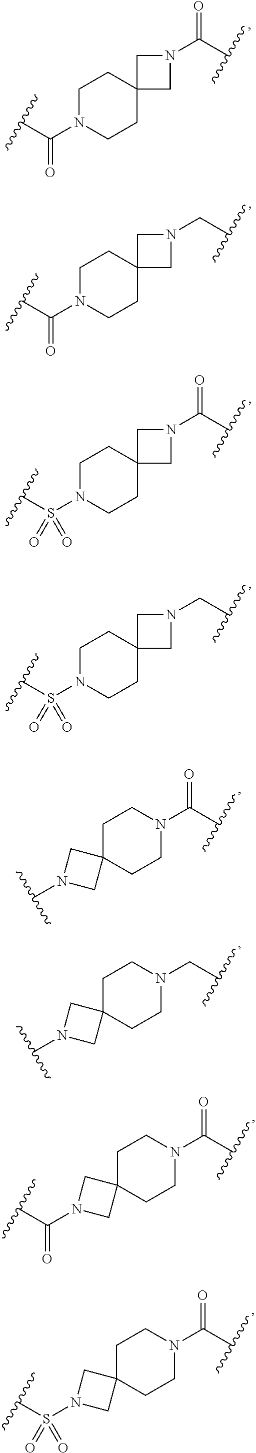

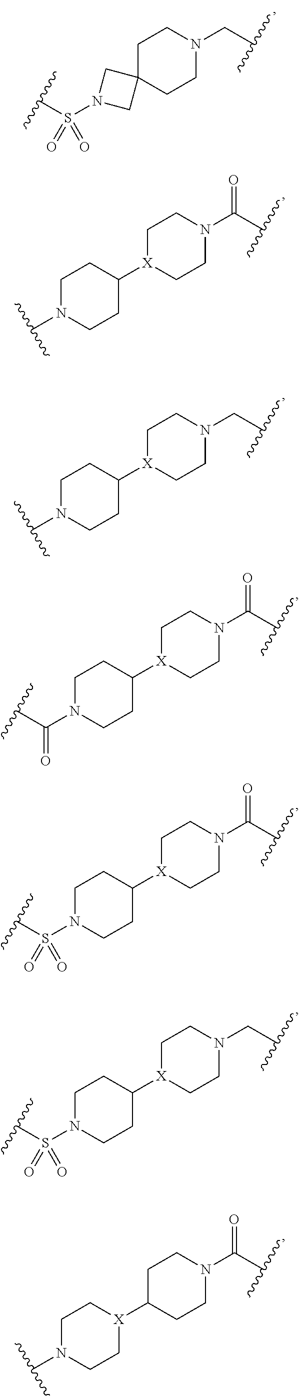

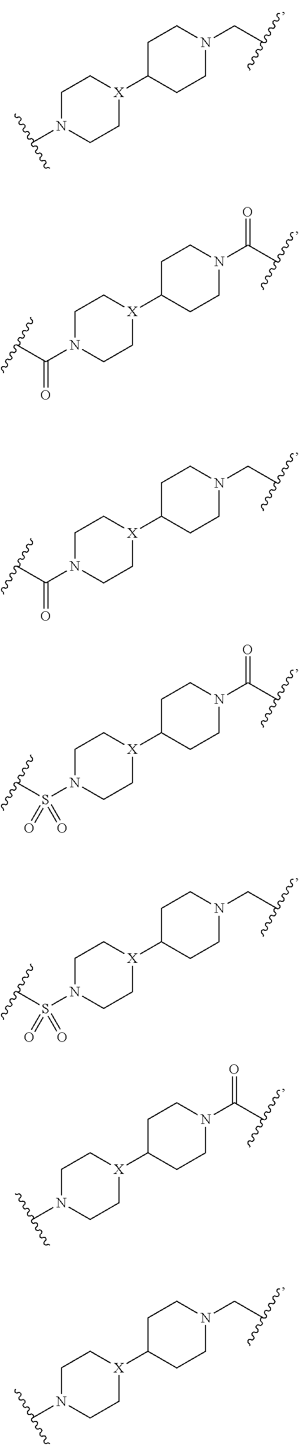

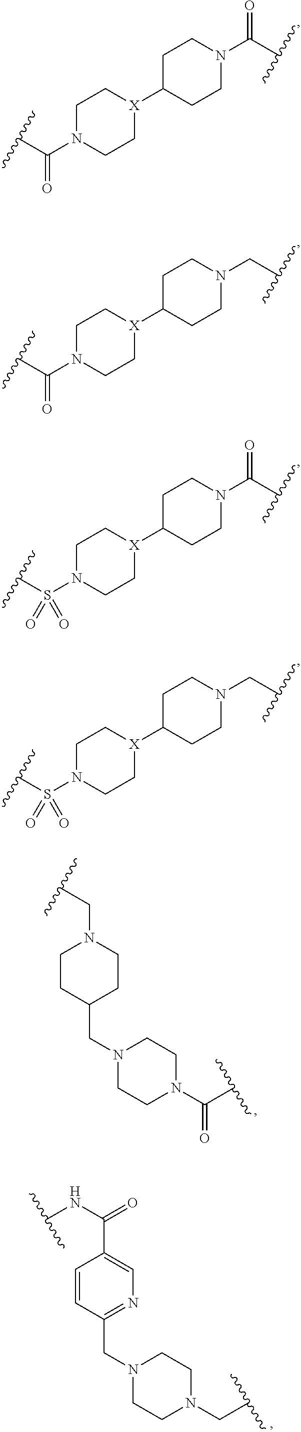

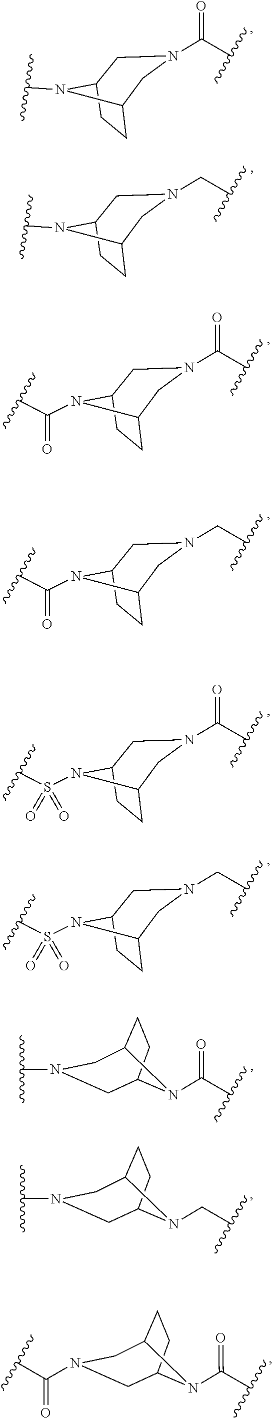

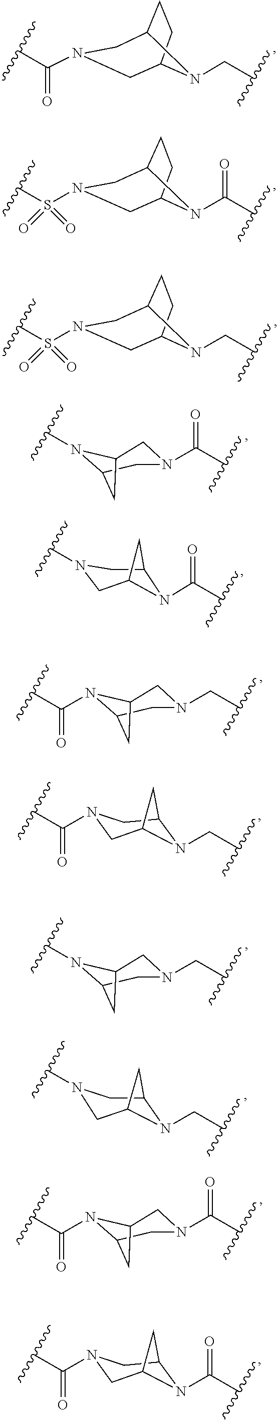

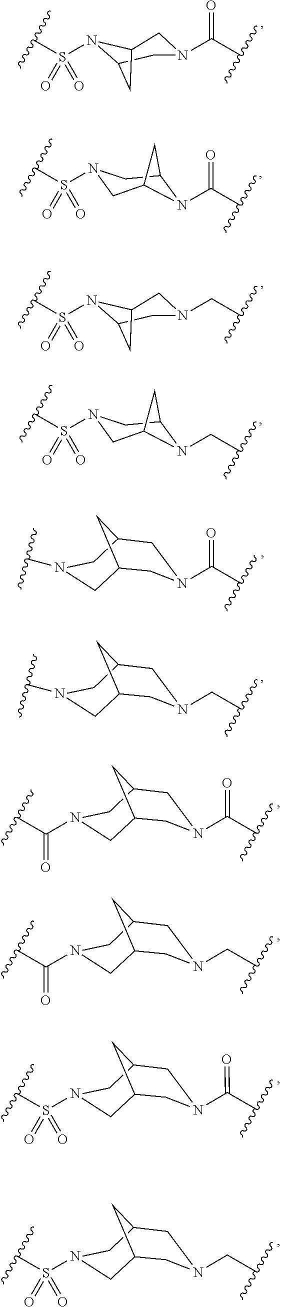

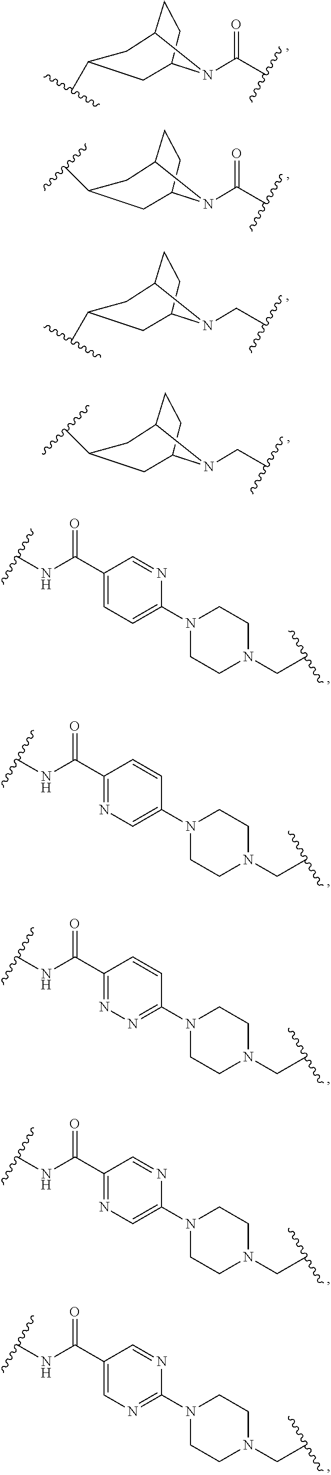

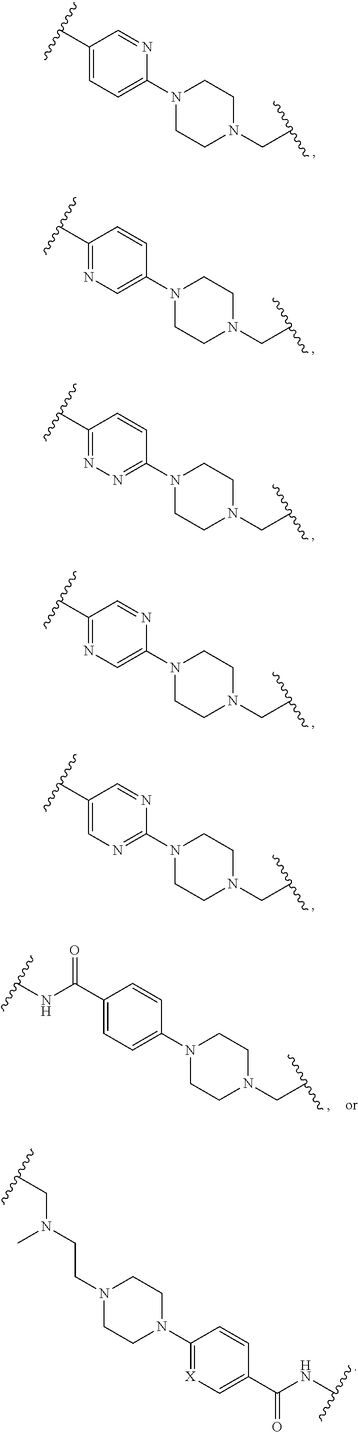

In one aspect, L₁ is

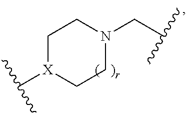

### X is N, and r is 1.

In some embodiments, L₁ is

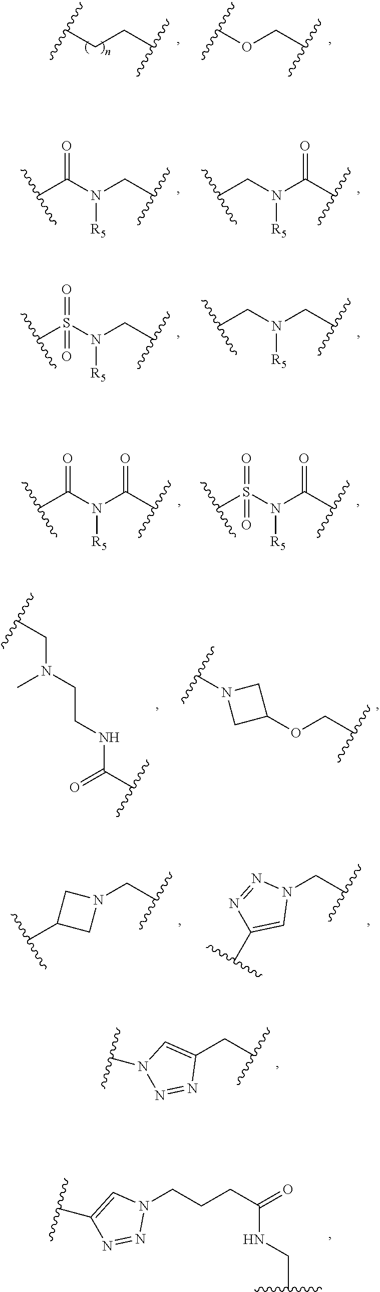

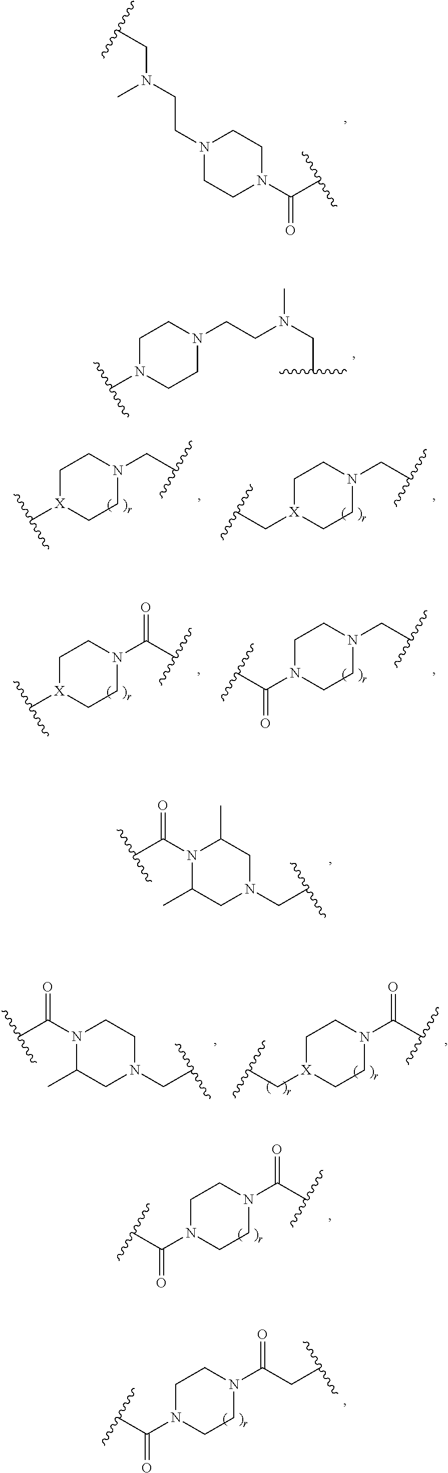

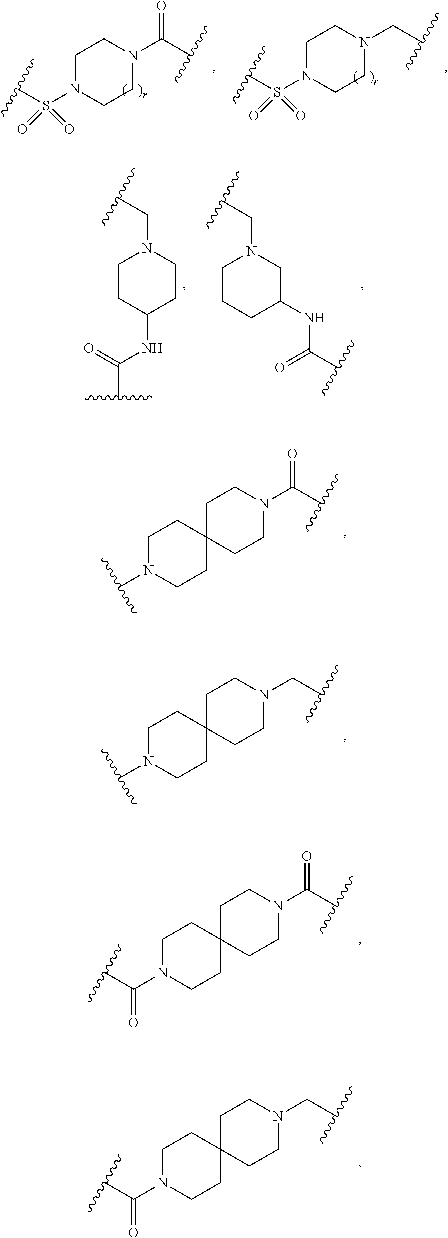

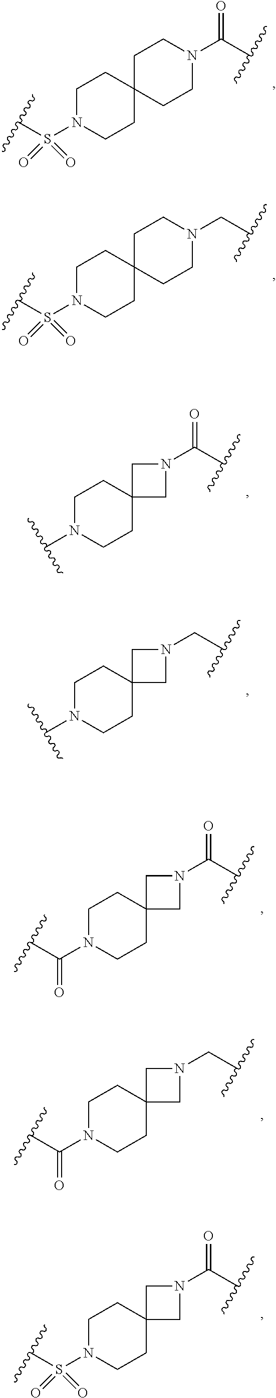

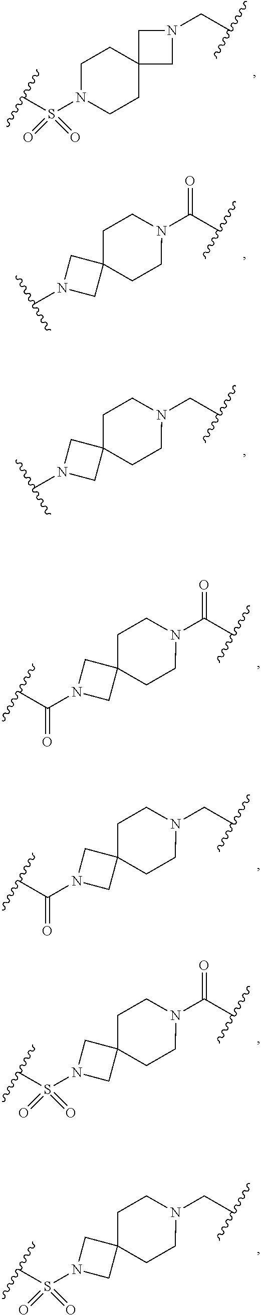

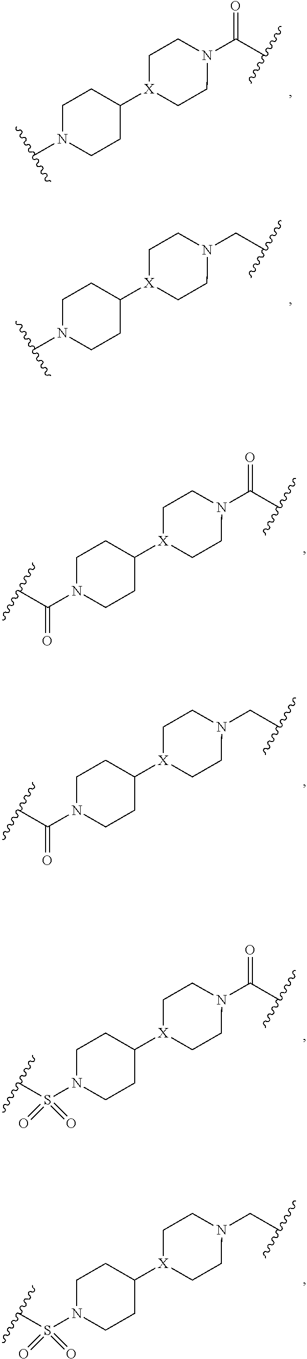

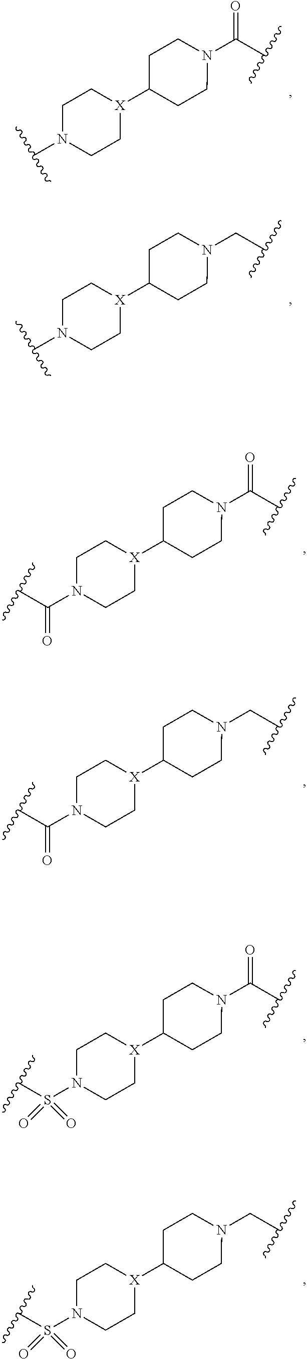

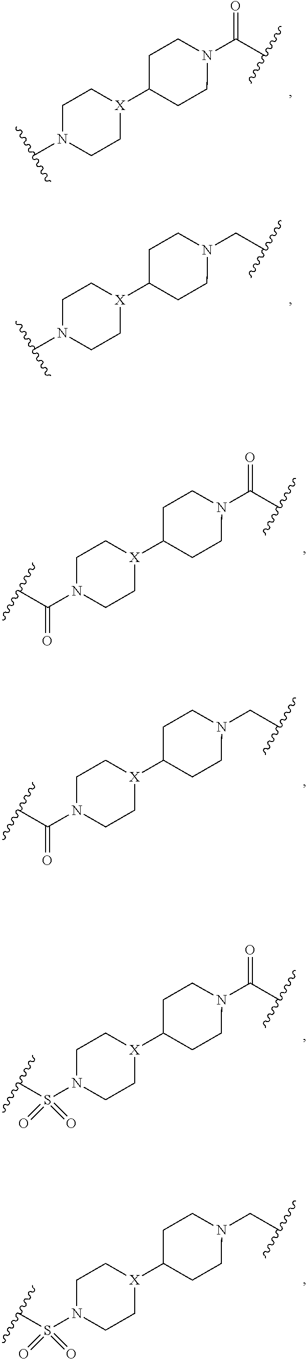

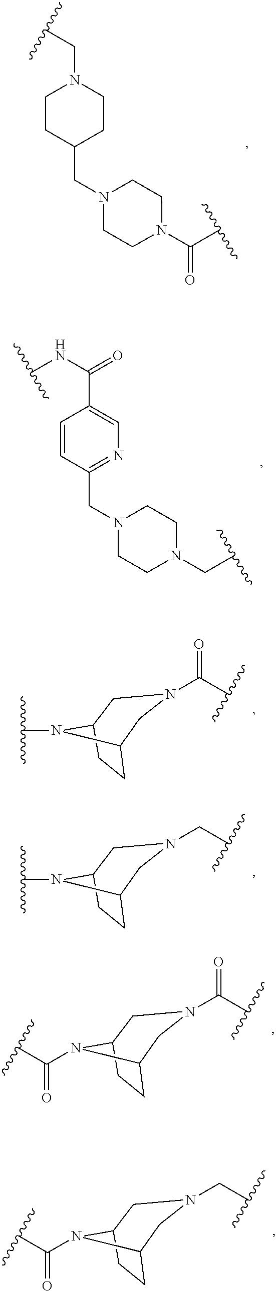 [SMILES: *CN1CCC(CN2CCN(C(*)=O)CC2)CC1 ; *CN1CCN(Cc2ccc(C(=O)N*)cn2)CC1 ; *C(=O)N1CC2CCC(C1)N2* ; *CN1CC2CCC(C1)N2* ; *C(=O)N1CC2CCC(C1)N2C(*)=O ; *CN1CC2CCC(C1)N2C(*)=O]

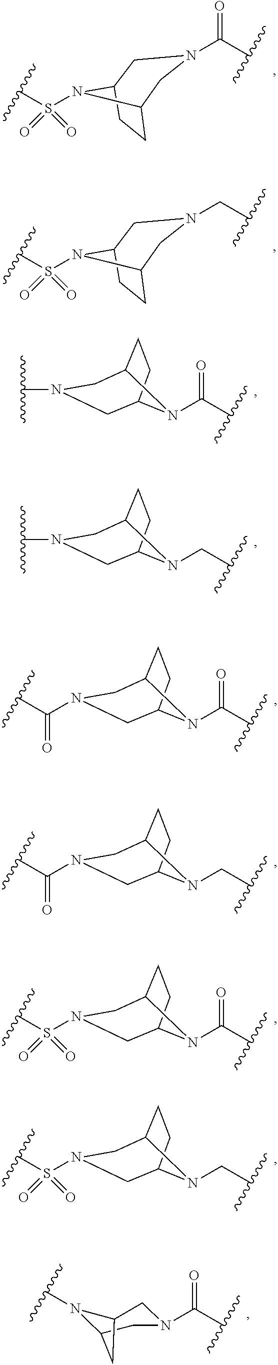

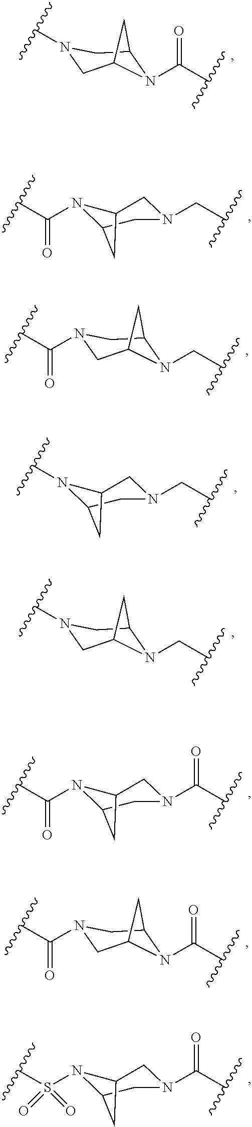

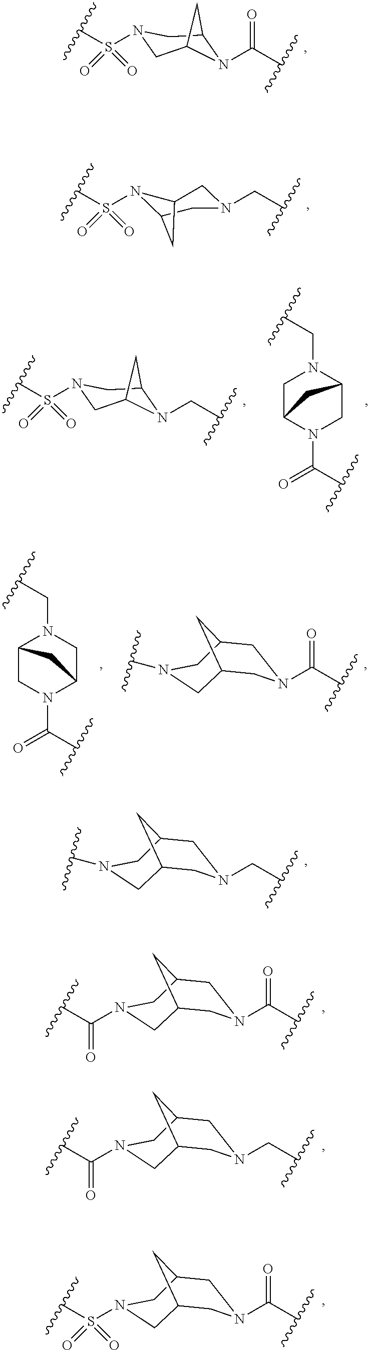

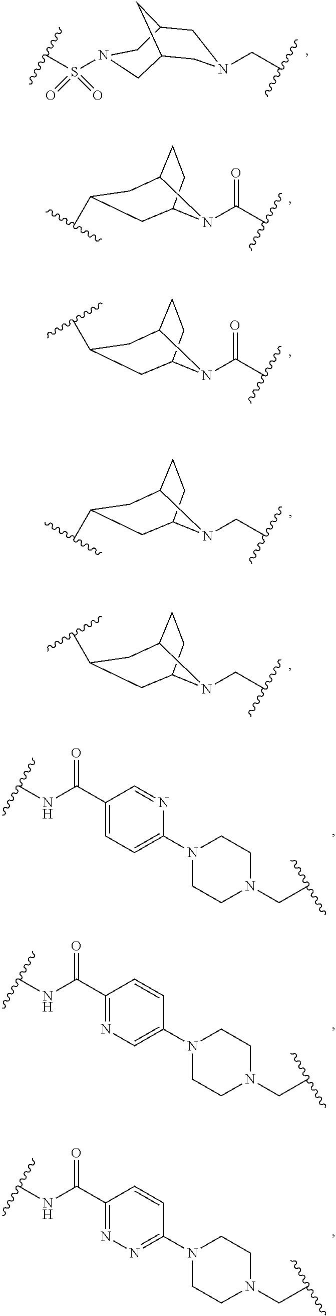

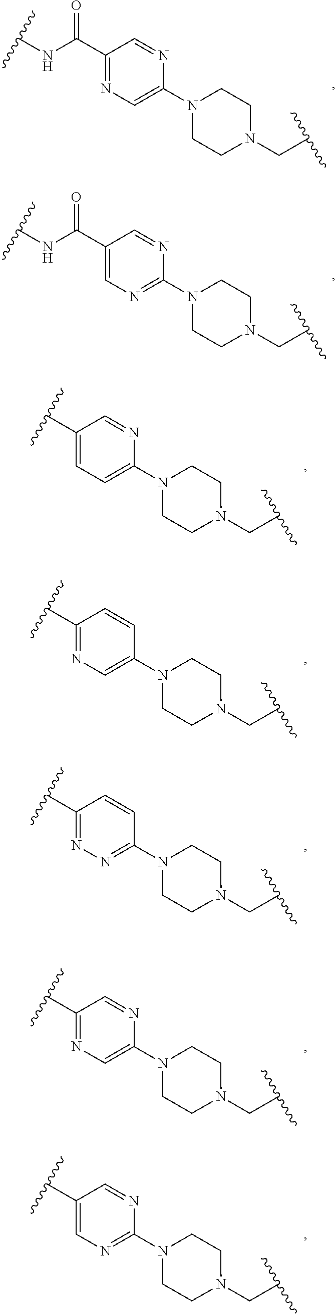

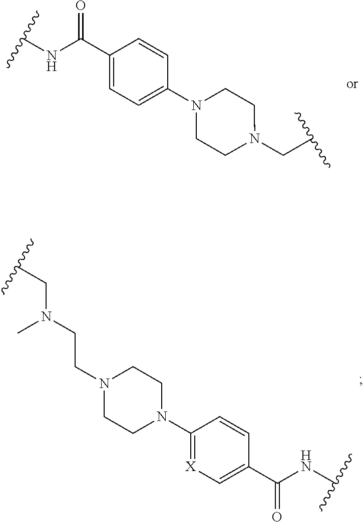

and
L₂ is a bond,

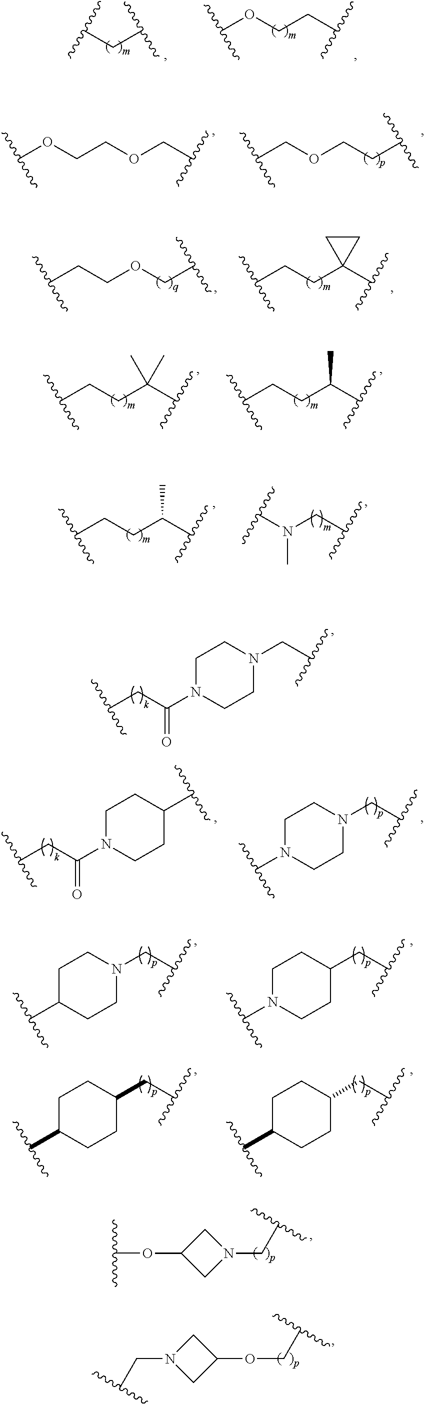

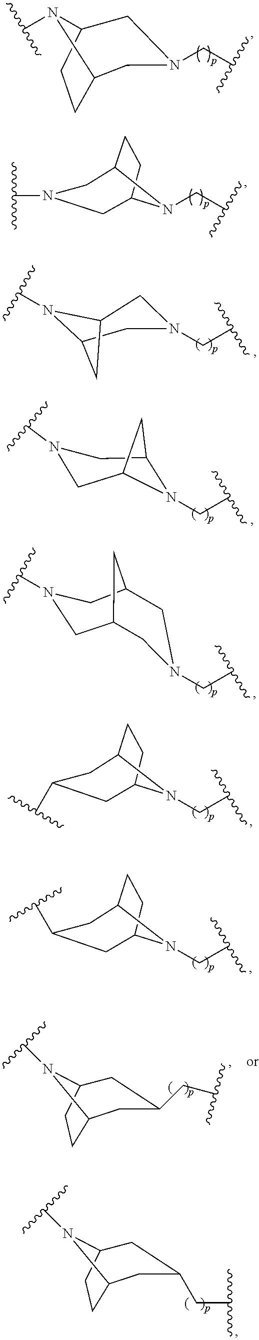

In some embodiments, L₂ is

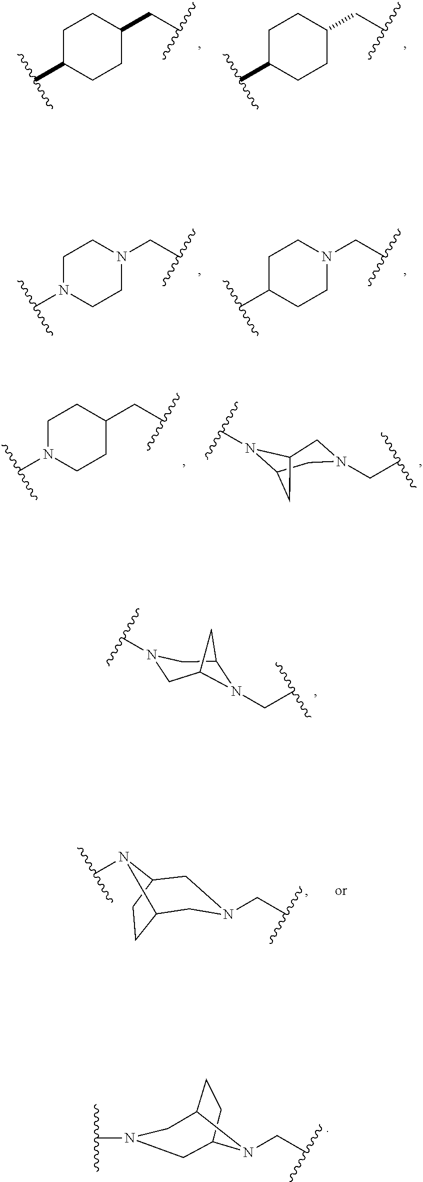

In some embodiments, L₂ is

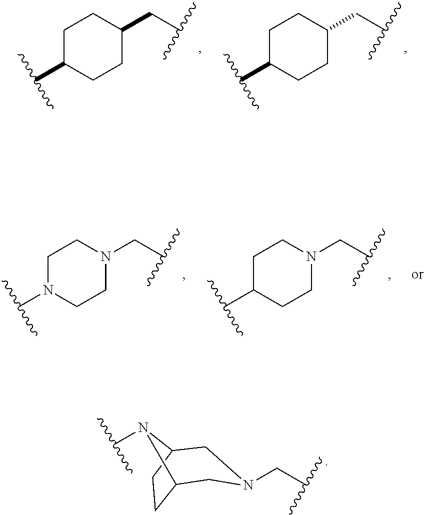 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

In some embodiments, L₂ is a bond,

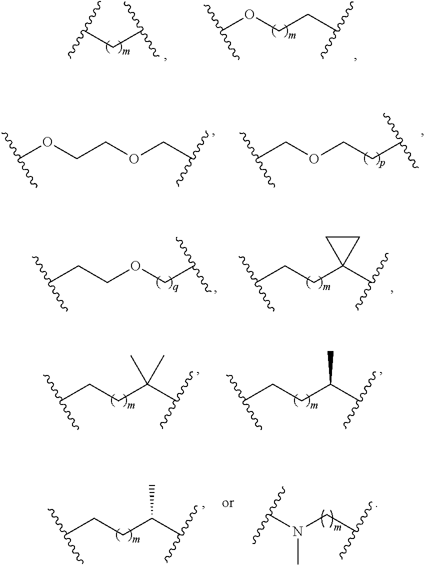

In some embodiments, L₂ is

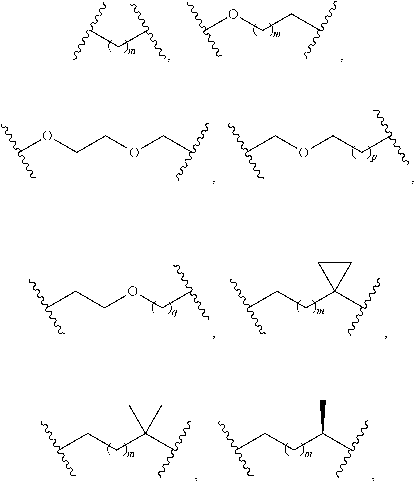

In some embodiments, L₂ is

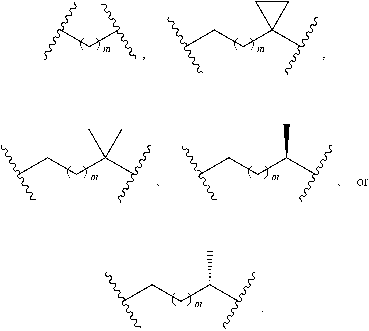

In some embodiments, L₂ is

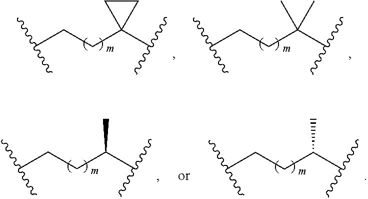

In some embodiments, L₂ is

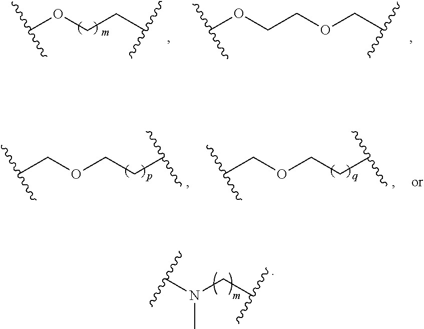

In some embodiments, L₂ is

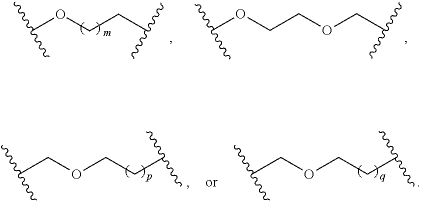

In some embodiments, L₁ is

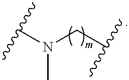

In some embodiments, L₂ is a bond. In some embodiments, L₂ is

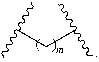

In some embodiments, L₂ is

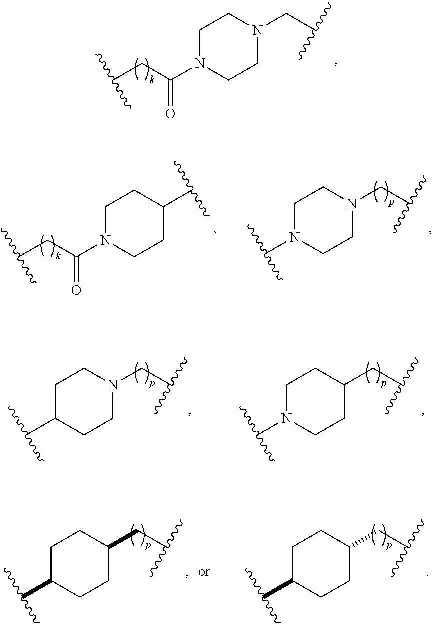

In some embodiments, L₂ is

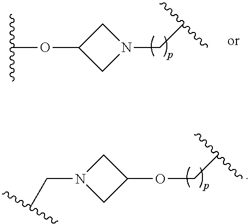

In some embodiments, L₂ is

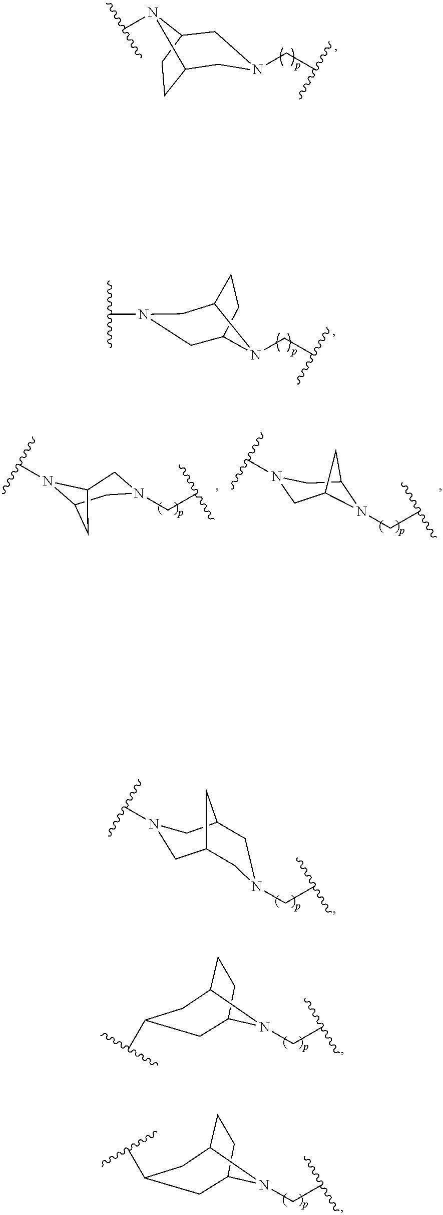

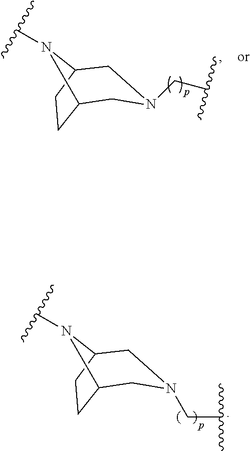

In some embodiments, L₂ is not

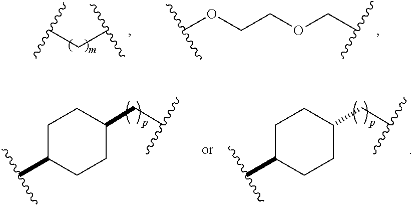

In some embodiments, L₂ is

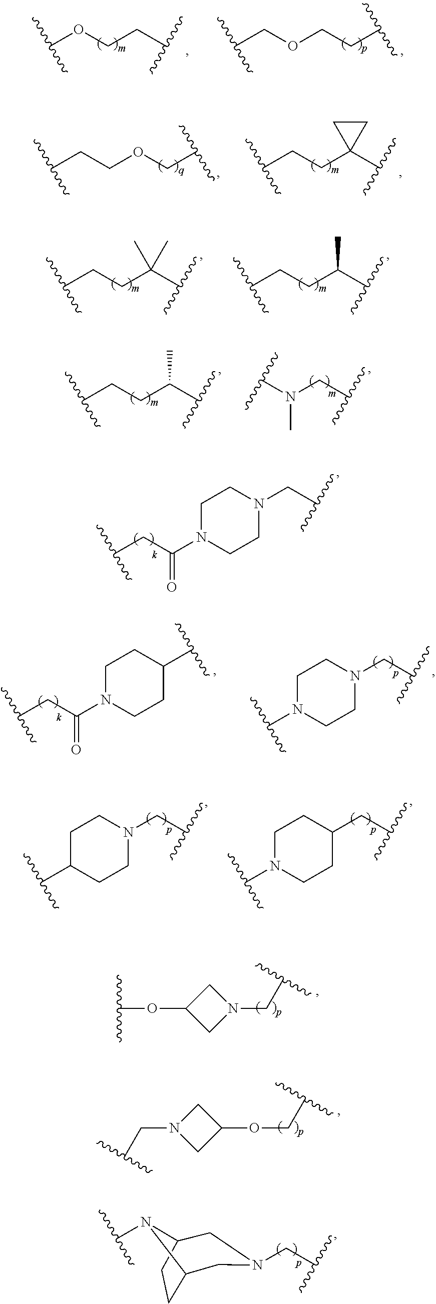

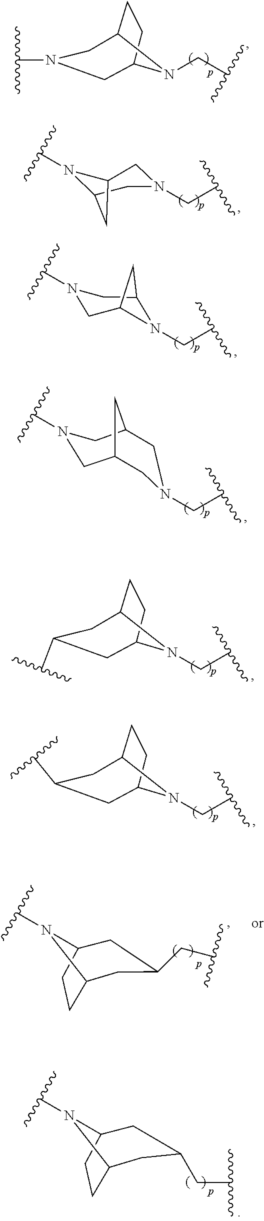

In some embodiments, L₂ is

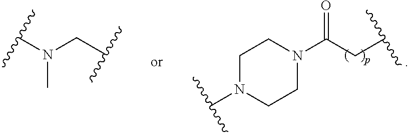

In one aspect, X is independently CH or N. In some embodiments, X is CH. In certain embodiments, X is N.

As defined herein, each k is independently 0, 1, 2, 3, 4, 5, or 6. In certain embodiments, each k is independently 0, 1, 2, 3, 4, or 5. In some embodiments, each k is independently 0, 1, 2, 3, or 4. In certain embodiments, each k is independently 0, 1, 2, or 3. In some embodiments, each k is independently 0, 1, or 2. In certain embodiments, each k is independently 3, 4, 5, or 6. In some embodiments, each k is independently 1, 2, 3, 4, 5, or 6. In certain embodiments, k is 0. In some embodiments, k is 1. In certain embodiments, k is 2. In some embodiments, k is 3. In certain embodiments, k is 4. In some embodiments, k is 5. In some embodiments, k is 6.

In one aspect, each m is independently 2, 3, 4, 5, 6, or 7. In some embodiments, each m is independently 2, 3, 4, 5, or 6. In certain embodiments, each m is independently 3, 4, 5, 6, or 7. In some embodiments, each m is independently 2, 3, 4, or 5. In certain embodiments, each m is independently 3, 4, 5, or 6. In some embodiments, each m is independently 4, 5, 6, or 7. In certain embodiments, each m is independently 2, 3, or 4. In some embodiments, each m is independently 3, 4, or 5. In certain embodiments, each m is independently 4, 5, or 6. In some embodiments, each m is 5, 6, or 7. In certain embodiments, each m is independently 2 or 3. In some embodiments, each m is independently 3 or 4. In certain embodiments, each m is independently 4 or 5. In some embodiments, each m is independently 5 or 6. In certain embodiments, each m is independently 6 or 7. In some embodiments, m is 1. In certain embodiments, m is 2. In some embodiments, m is 3. In certain embodiments, m is 4. In some embodiments, m is 5. In certain embodiments, m is 6. In some embodiments, m is 7.

In one aspect, each n is independently 0, 1, 2, 3, 4, 5, 6, 7, 8, 9, or 10. In some embodiments, each n is independently 0, 1, 2, 3, 4, 5, 6, 7, 8, or 9. In certain embodiments, each n is independently 1, 2, 3, 4, 5, 6, 7, 8, 9, or 10. In some embodiments, each n is independently 0, 1, 2, 3, 4, 5, 6, 7, or 8. In some embodiments, each n is independently 1, 2, 3, 4, 5, 6, 7, 8, or 9. In certain embodiments, each n is independently 2, 3, 4, 5, 6, 7, 8, 9, or 10. In some embodiments, each n is independently 0, 1, 2, 3, 4, or 5. In some embodiments, each n is independently 1, 2, 3, 4, 5, or 6. In certain embodiments, each n is independently 2, 3, 4, 5, 6, or 7. In some embodiments, each n is independently 3, 4, 5, 6, 7, or 8. In certain embodiments, each n is independently 4, 5, 6, 7, 8, or 9. In some embodiments, each n is independently 5, 6, 7, 8, 9, or 10. In certain embodiments, each n is independently 0, 1, 2, 3, or 4. In some embodiments, each n is independently 1, 2, 3, 4, or 5. In some embodiments, each n is independently 2, 3, 4, 5, or 6. In certain embodiments, each n is independently 3, 4, 5, 6, or 7. In some embodiments, each n is independently 0, 1, 2, or 3. In some embodiments, each n is independently 1, 2, 3, or 4. In some embodiments, each n is independently 2, 3, 4, or 5. In certain embodiments, each n is independently 3, 4, 5, or 6. In some embodiments, each n is independently 4, 5, 6, or 7. In certain embodiments, each n is independently 5, 6, 7, or 8. In some embodiments, each n is independently 6, 7, 8, or 9. In certain embodiments, each n is independently 7, 8, 9, or 10. In some embodiments, each n is independently 0, 1, or 2. In certain embodiments, each n is independently 1, 2, or 3. In certain embodiments, each n is independently 2, 3, or 4. In some embodiments, each n is independently 3, 4, or 5. In certain embodiments, each n is independently 4, 5, or 6. In some embodiments, each n is 5, 6, or 7. In certain embodiments, each n is independently 5, 6, or 7. In some embodiments, each n is 7, 8, or 9. In certain embodiments, each n is independently 8, 9, or 10. In some embodiments, n is 0 or 1. In some embodiments, n is 1 or 2. In certain embodiments, each n is independently 2 or 3. In some embodiments, each n is independently 3 or 4. In certain embodiments, each n is independently 4 or 5. In some embodiments, each n is independently 5 or 6. In certain embodiments, each n is independently 6 or 7. In some embodiments, each n is independently 7 or 8. In certain embodiments, each n is independently 8 or 9. In some embodiments, each n is independently 9 or 10. In certain embodiments, n is 0. In some embodiments, n is 1. In certain embodiments, n is 2. In some embodiments, n is 3. In certain embodiments, n is 4. In some embodiments, n is 5. In certain embodiments, n is 6. In some embodiments, n is 7. In certain embodiments, n is 8. In some embodiments, n is 9. In certain embodiments, n is 10.

In one aspect, each p is independently 0, 1, 2, 3, or 4. In some embodiments, each p Is independently 0, 1, 2, or 3. In certain embodiments, each p is independently 1, 2, 3, or 4. In some embodiments, each p is independently 0, 1, or 2. In certain embodiments, each p is independently 1, 2, or 3. In some embodiments, each p is independently 2, 3, or 4. In certain embodiments, each p is independently 0 or 1. In some embodiments, each p is independently 1 or 2. In certain embodiments, each p is independently 2 or 3. In some embodiments, each p is independently 3 or 4. In certain embodiments, p is 0. In some embodiments, p is 1. In certain embodiments, p is 2. In some embodiments, p is 3. In certain embodiments, p is 4.

In one aspect, each q is independently 1, 2, or 3. In certain embodiments, each q is independently 1 or 2. In some embodiments, each q is independently 2 or 3. In certain embodiments, q is 1. In some embodiments, q is 2. In certain embodiments, q is 3.

In one aspect, r is independently 1 or 2. In some embodiments, r is 1. In certain embodiments, r is 2.

In some embodiments, L₁ is

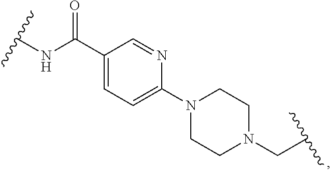 [SMILES: *CN1CCN(c2ccc(C(=O)N*)cn2)CC1]

and L₂ is a bond or

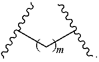

In certain embodiments, L₁ is

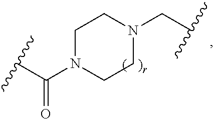

### and L₂ is

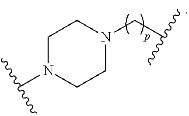

In certain embodiments, L₁ is

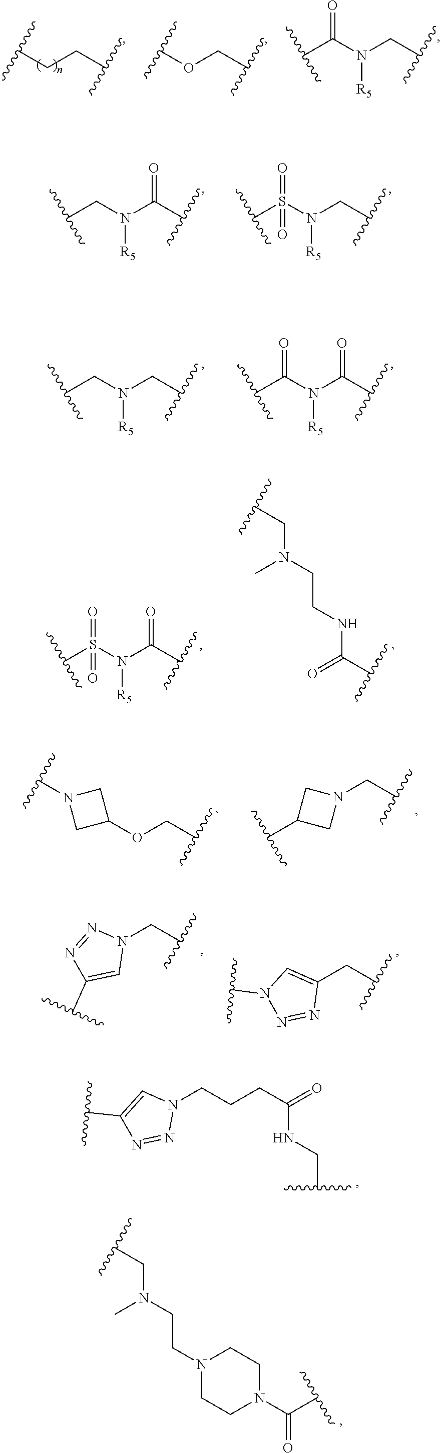

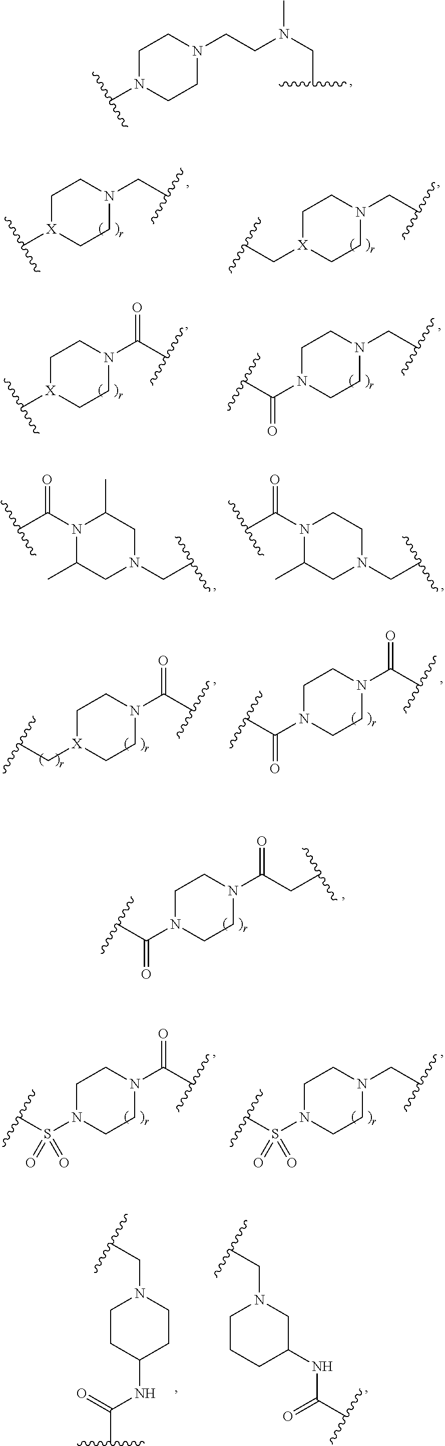

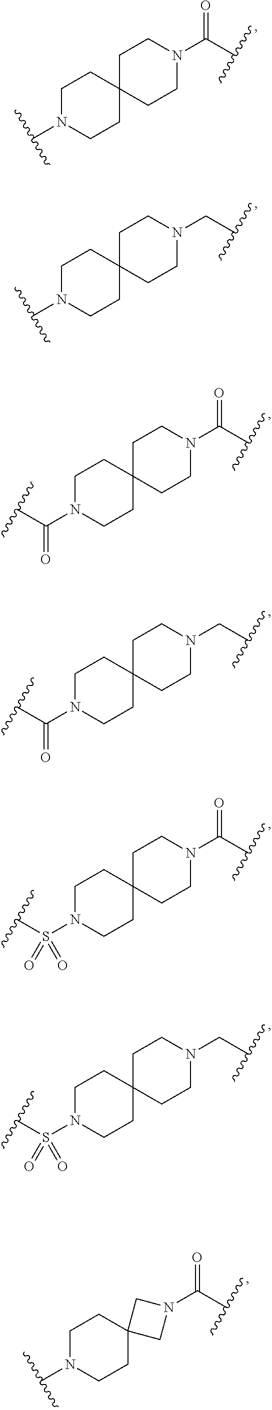

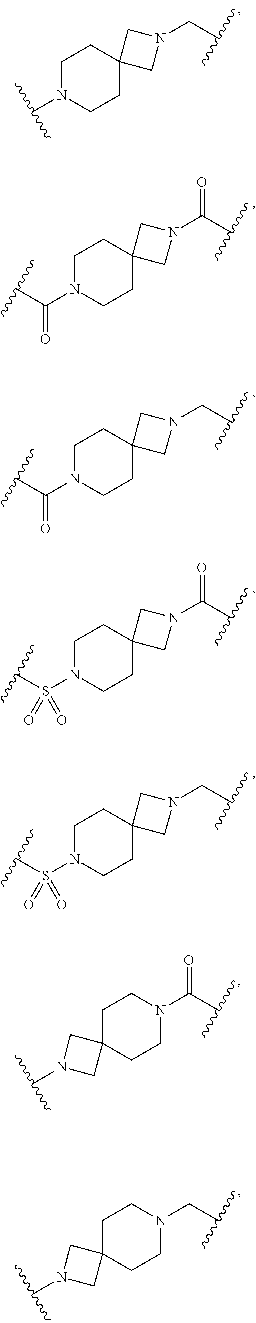

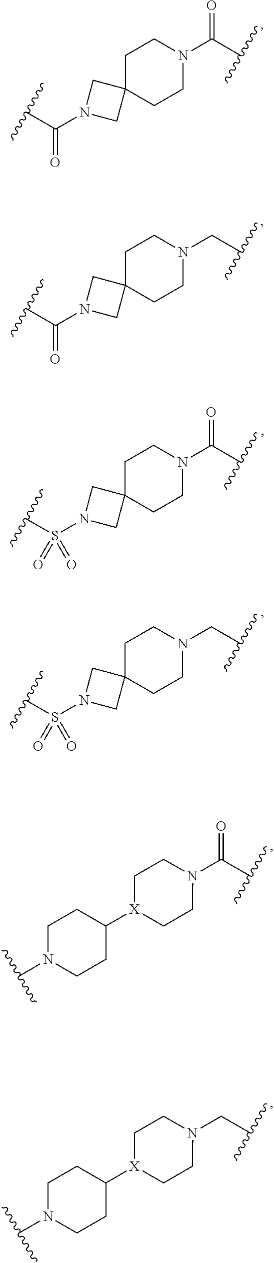

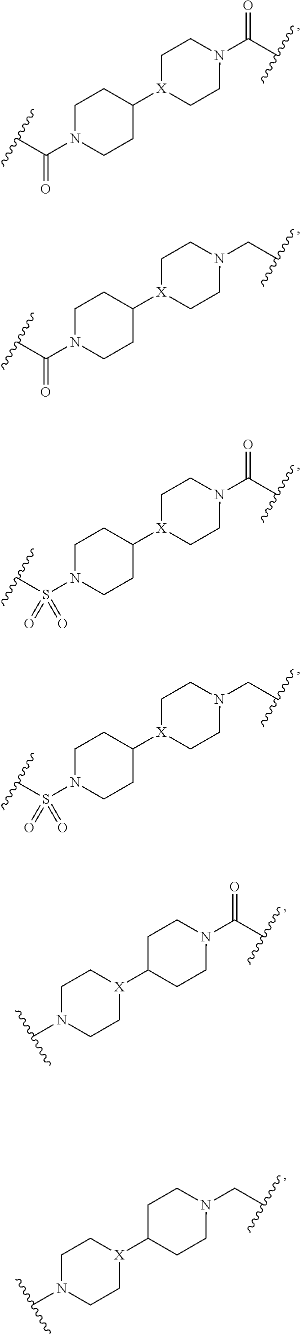

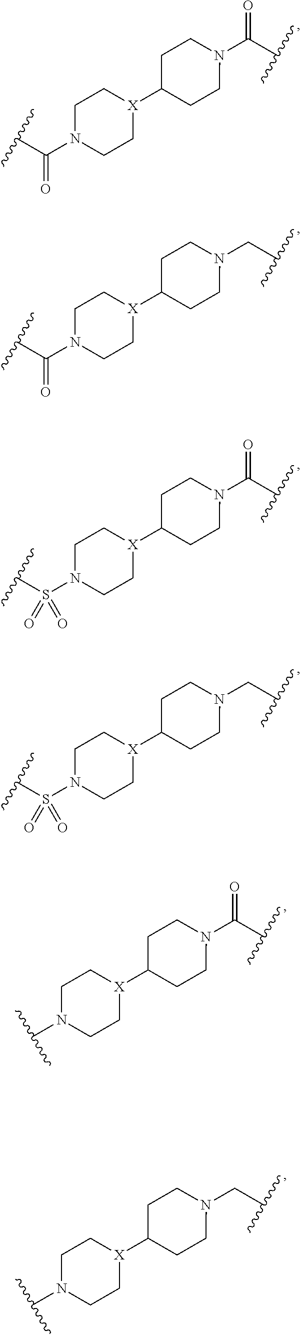

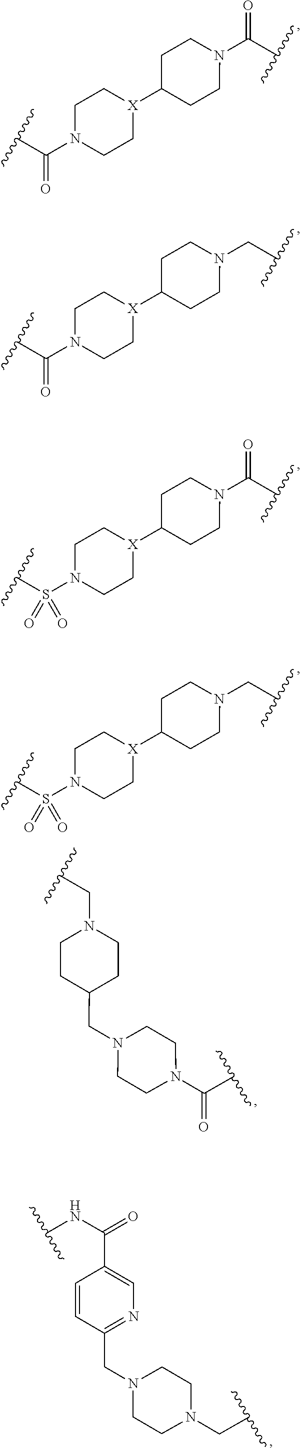

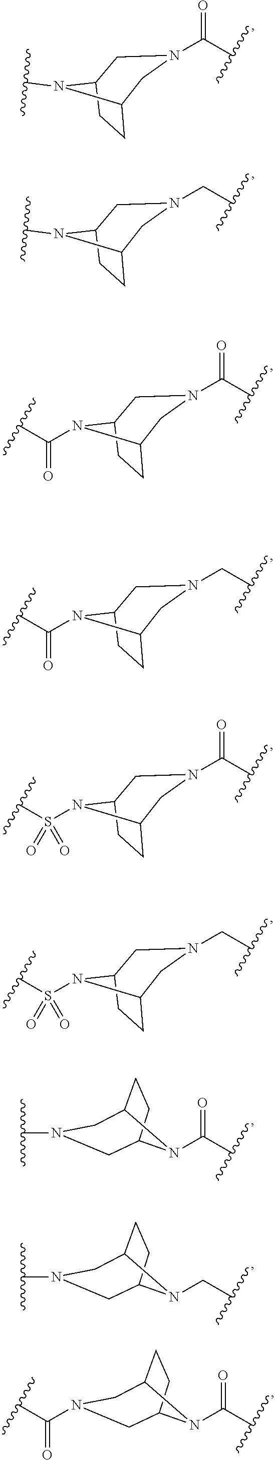

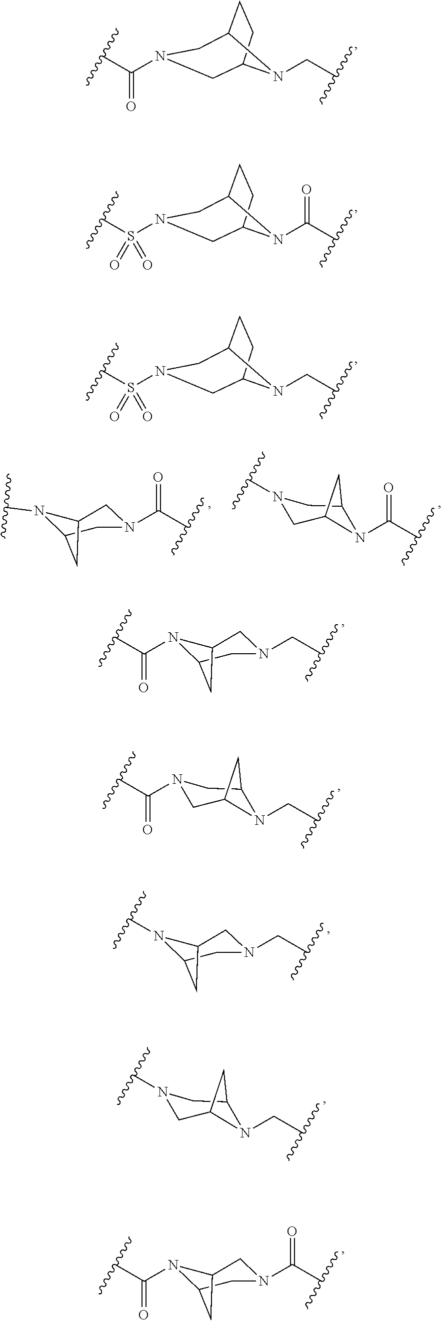

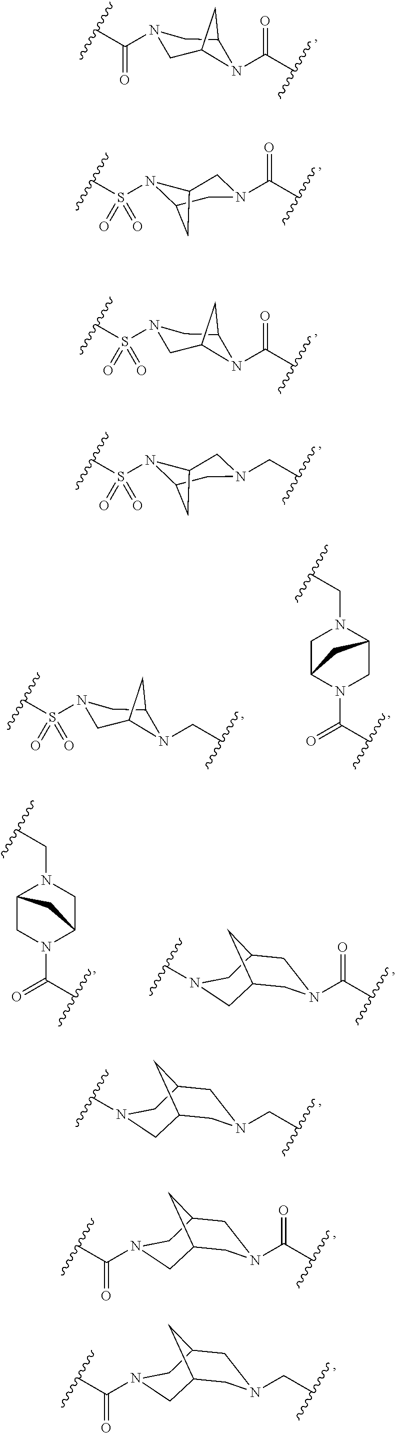

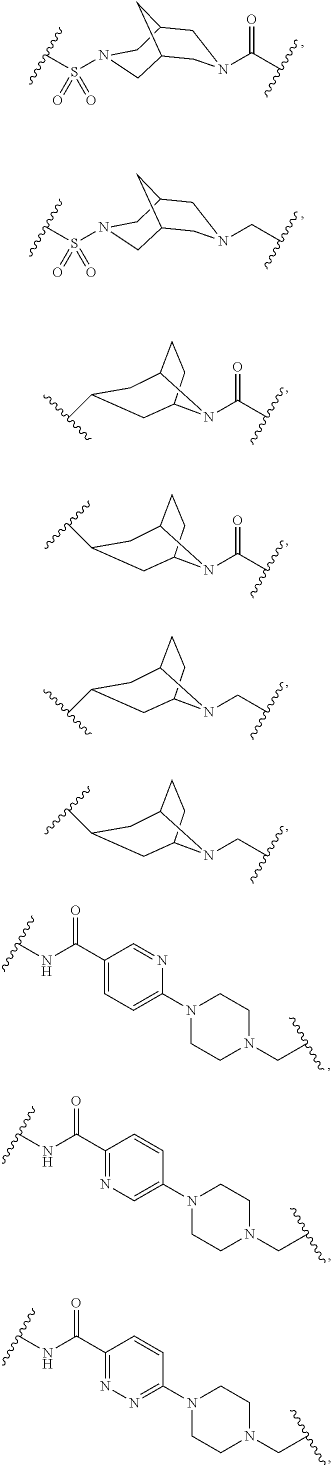

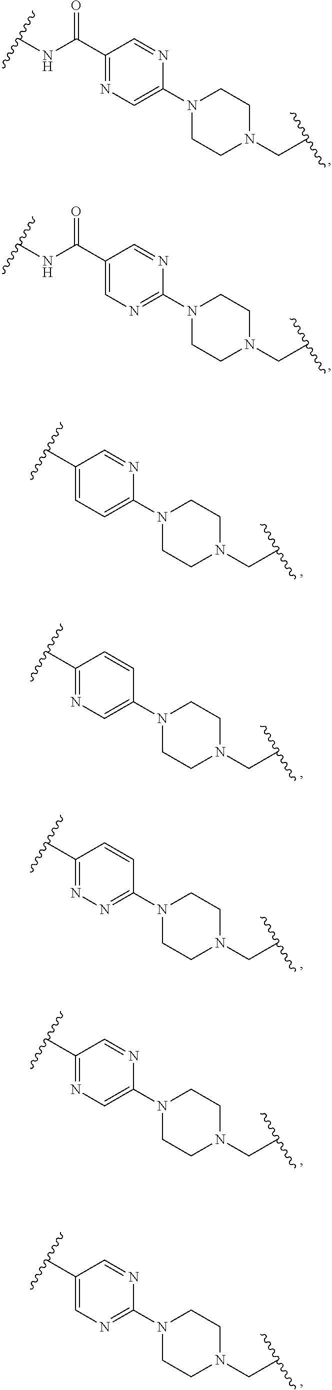

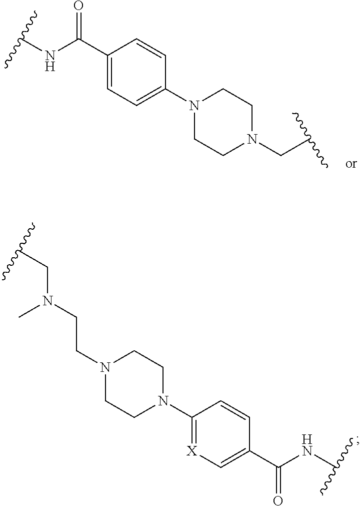

### and L₂ is

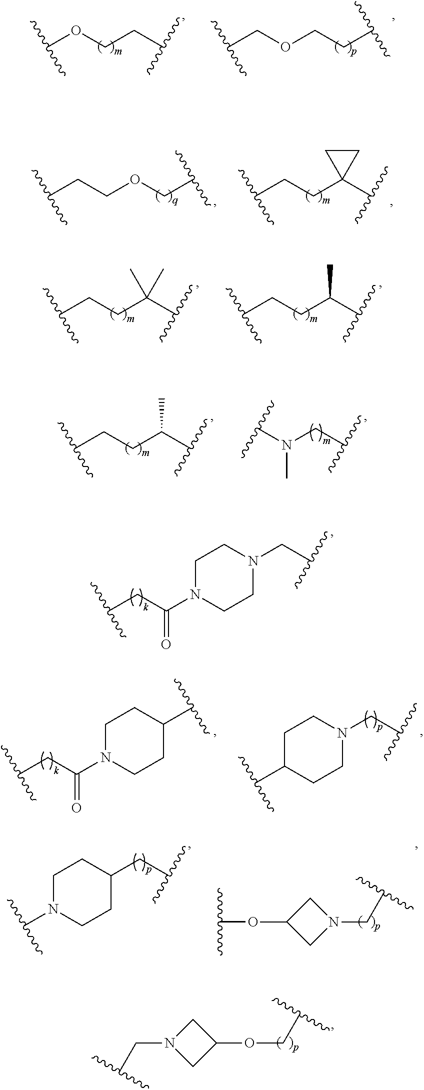

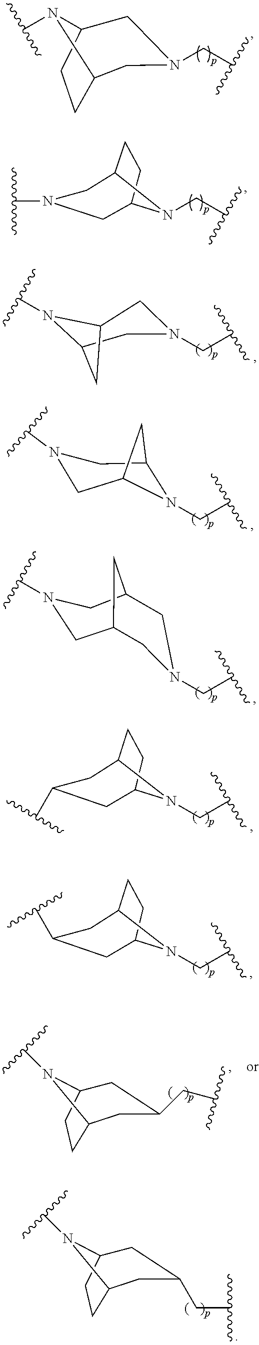

In certain embodiments, L₁ is

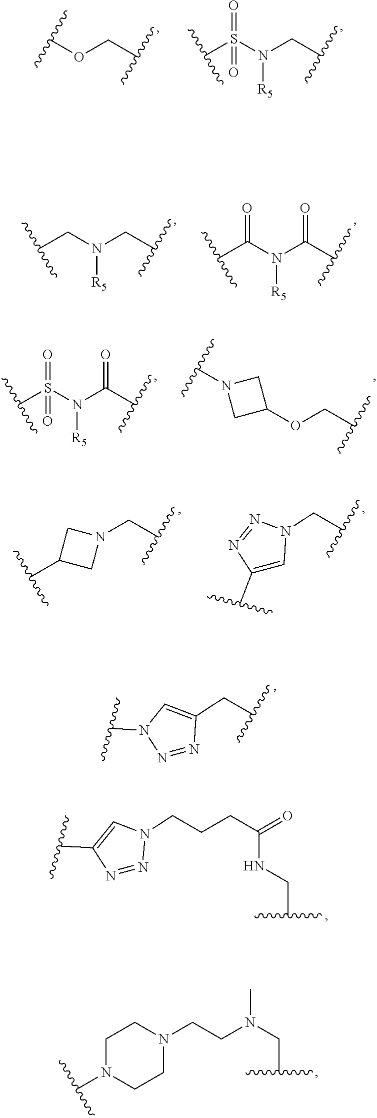

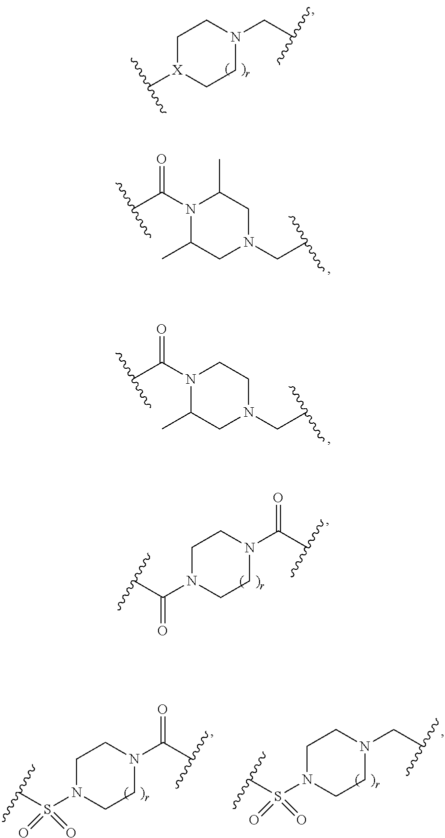

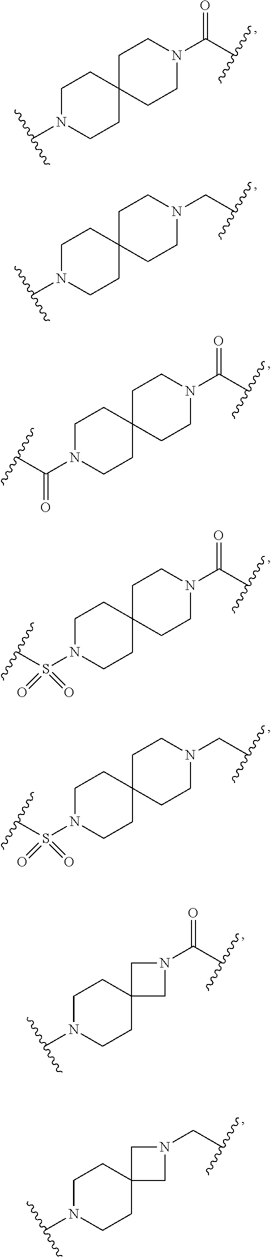

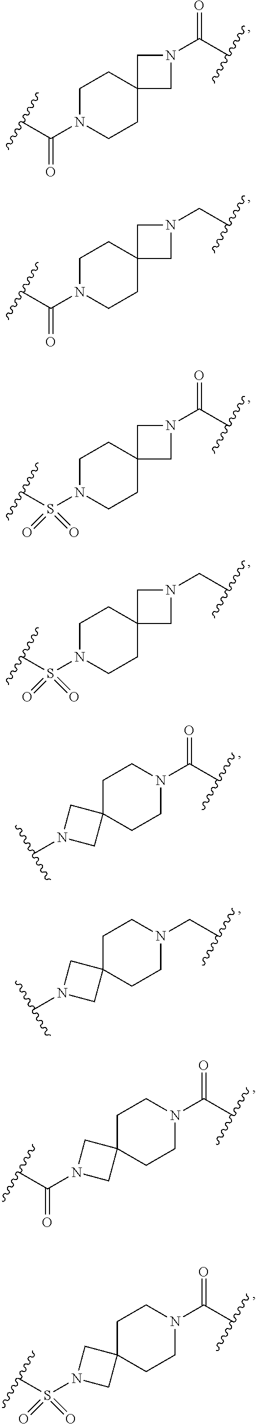

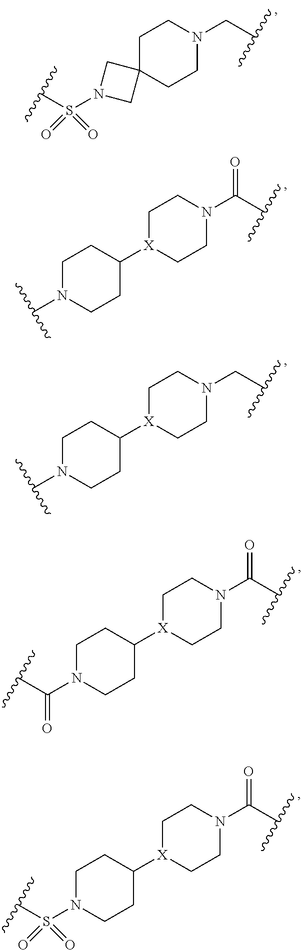

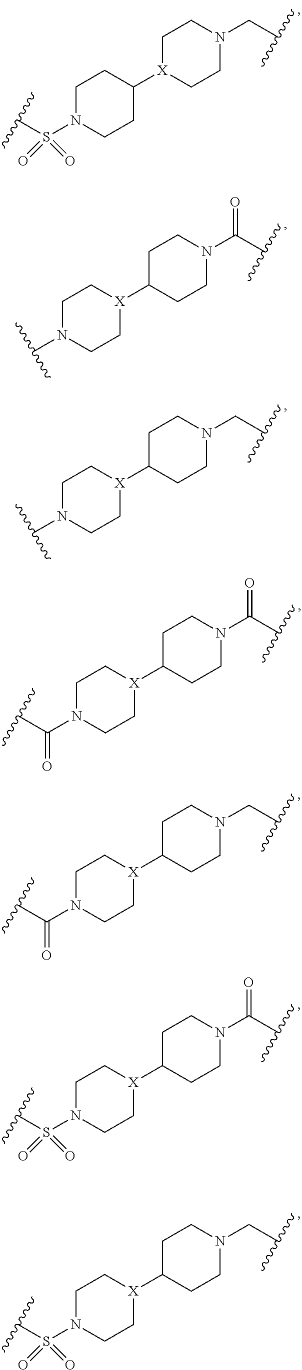

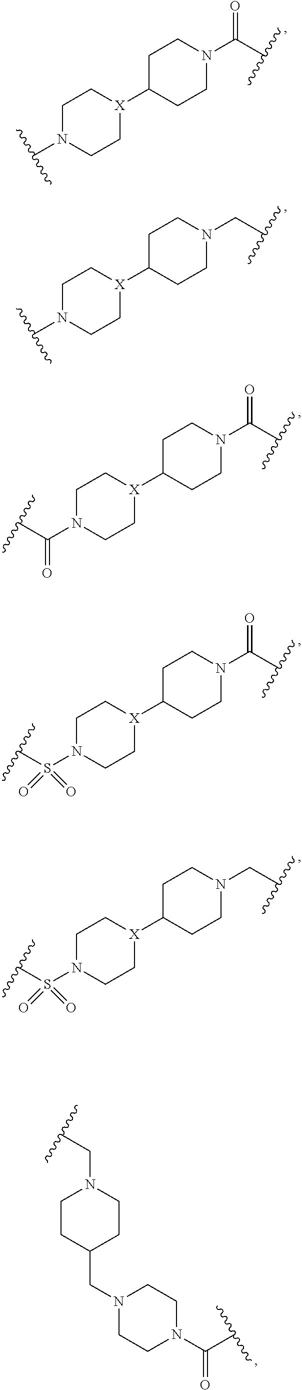

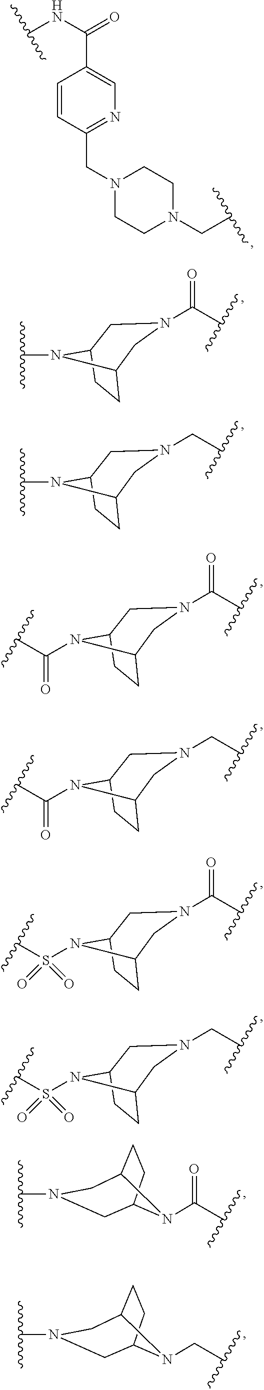

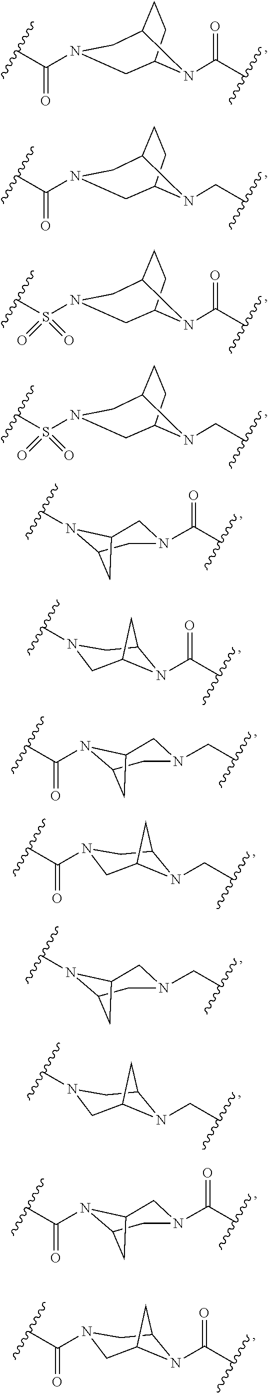

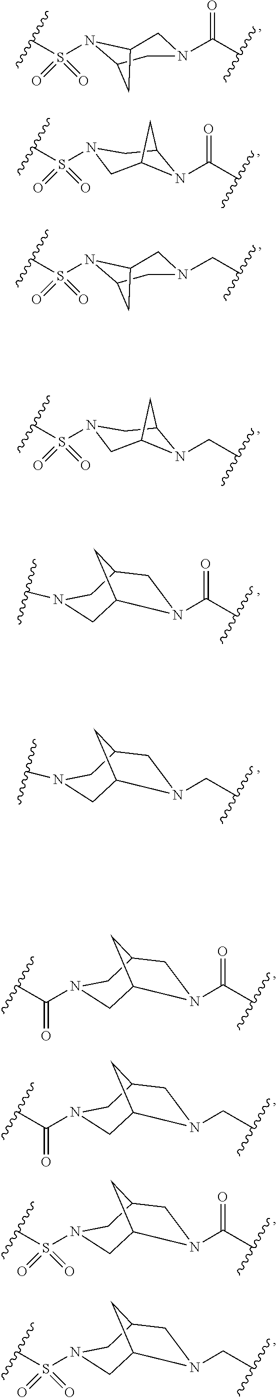

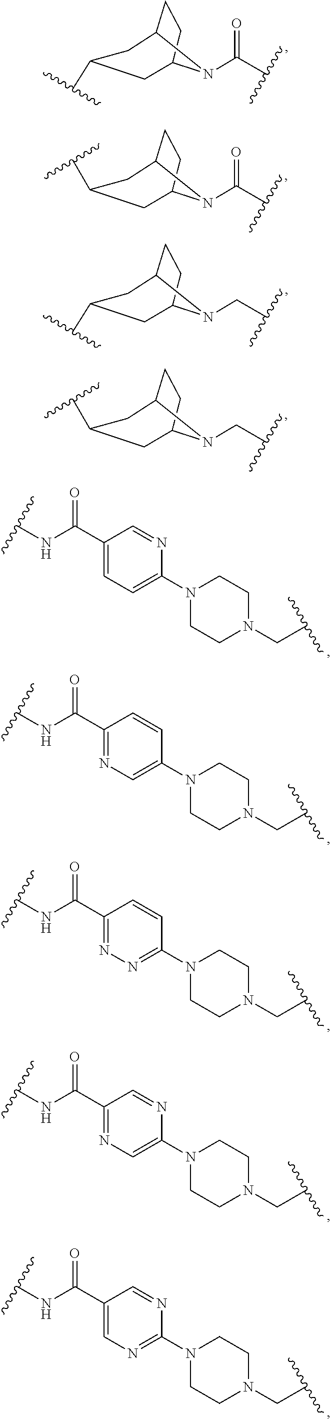

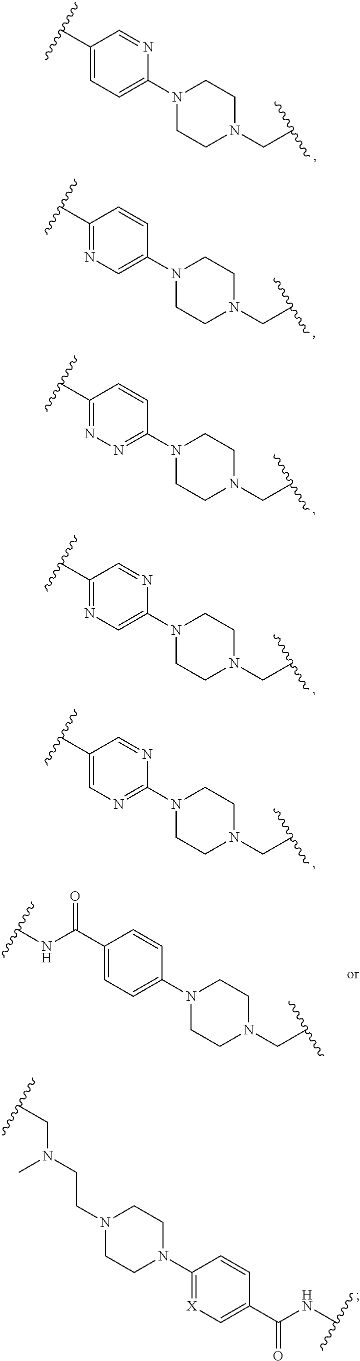

and L₂ is a bond,

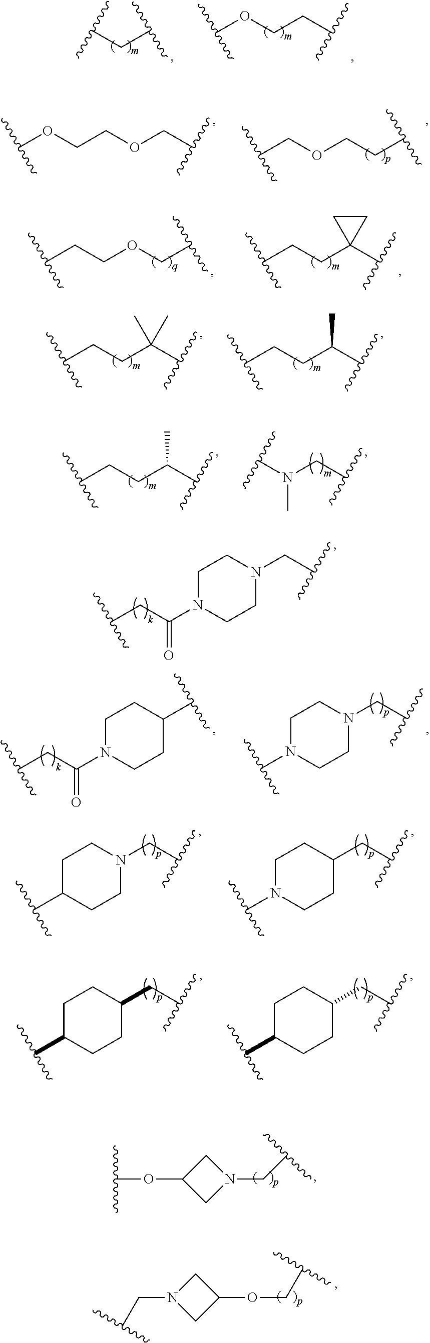

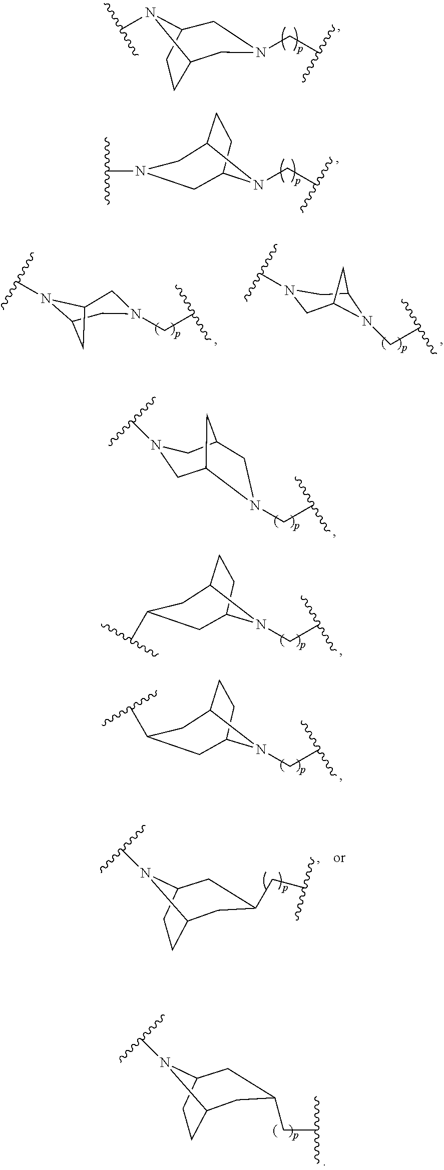

In another aspect, the compound of Formula (II) is:

| Compound | |
|---|---|
| # | Formula |
| 1 | 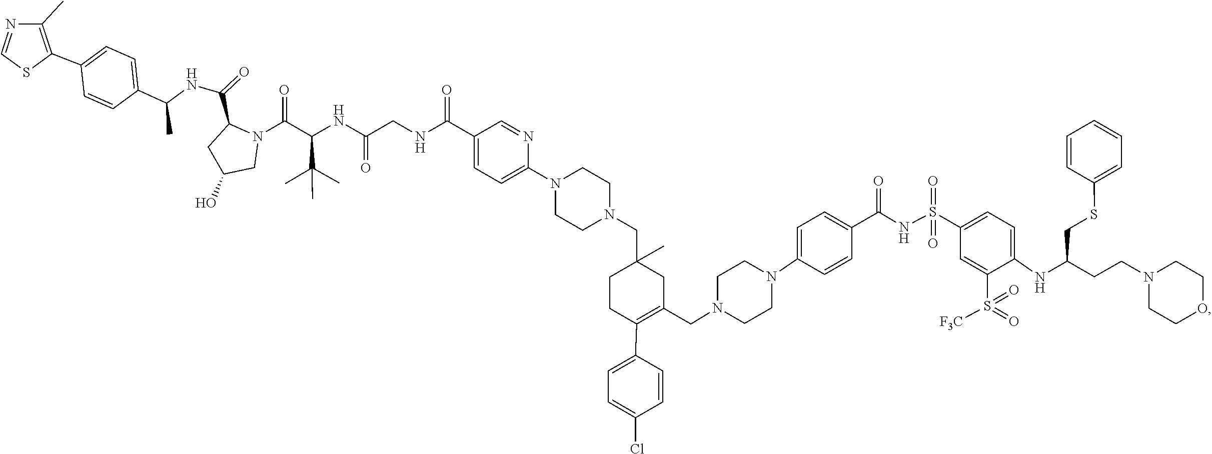 [SMILES: Cc1ncsc1-c1ccc([C@H](C)NC(=O)[C@@H]2C[C@@H](O)CN2C(=O)[C@@H](NC(=O)CNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 2 | 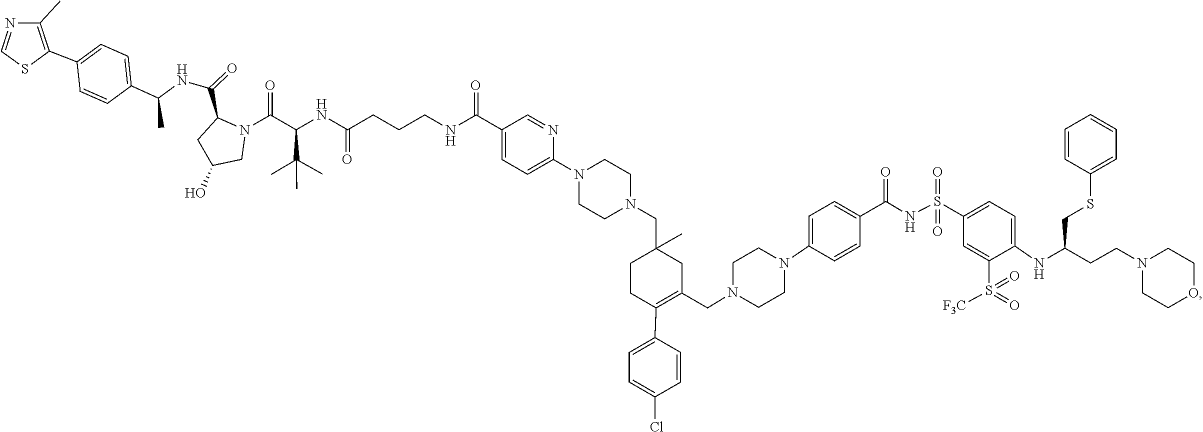 [SMILES: Cc1ncsc1-c1ccc([C@H](C)NC(=O)[C@@H]2C[C@@H](O)CN2C(=O)[C@@H](NC(=O)CCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 3 | 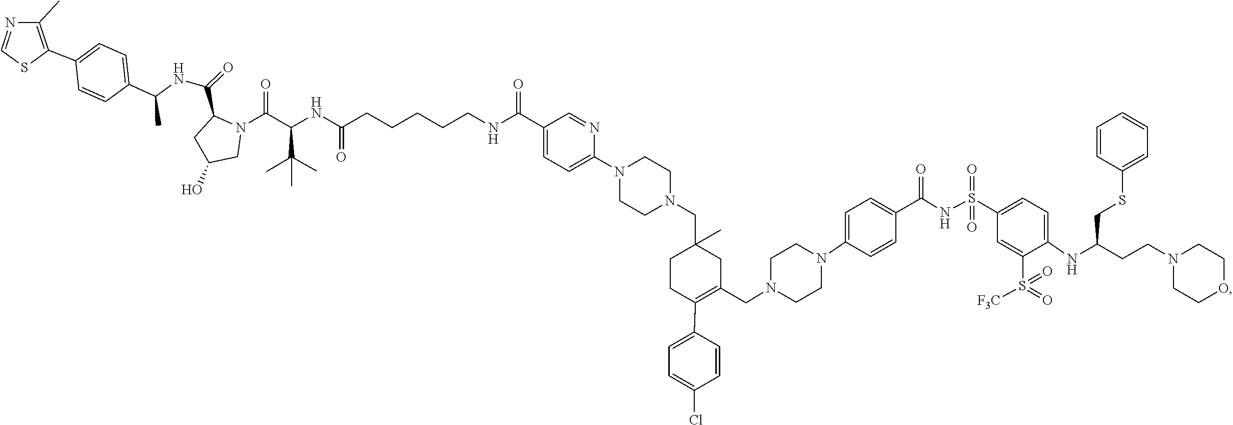 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 4 | 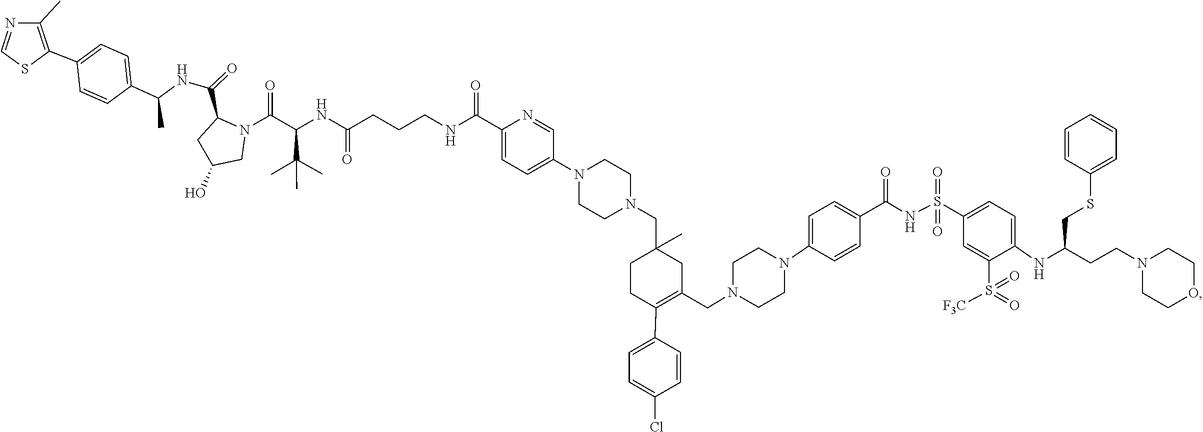 [SMILES: Cc1ncsc1-c1ccc([C@H](C)NC(=O)[C@@H]2C[C@@H](O)CN2C(=O)[C@@H](NC(=O)CCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cn2)C(C)(C)C)cc1] |
| 5 | 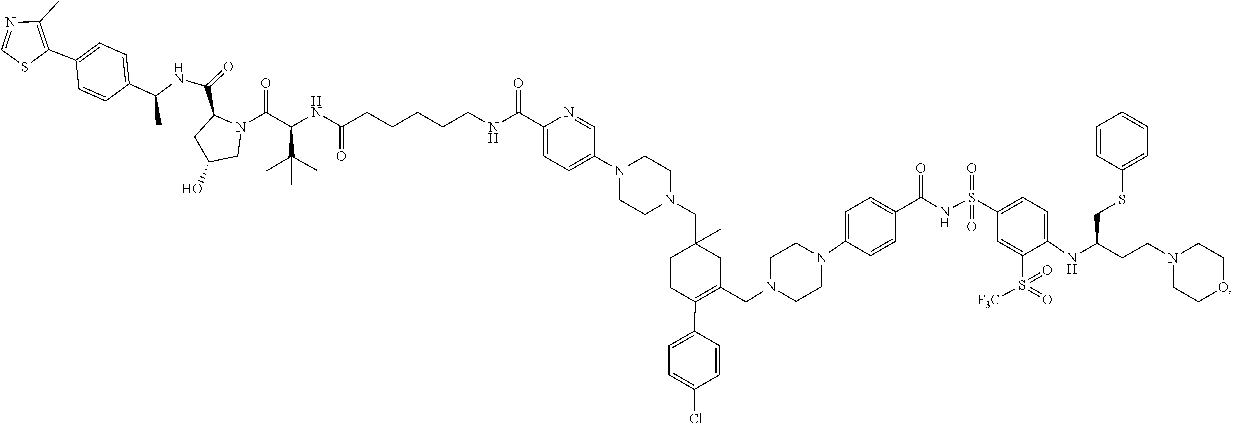 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cn2)C(C)(C)C)cc1] |
| 6 | 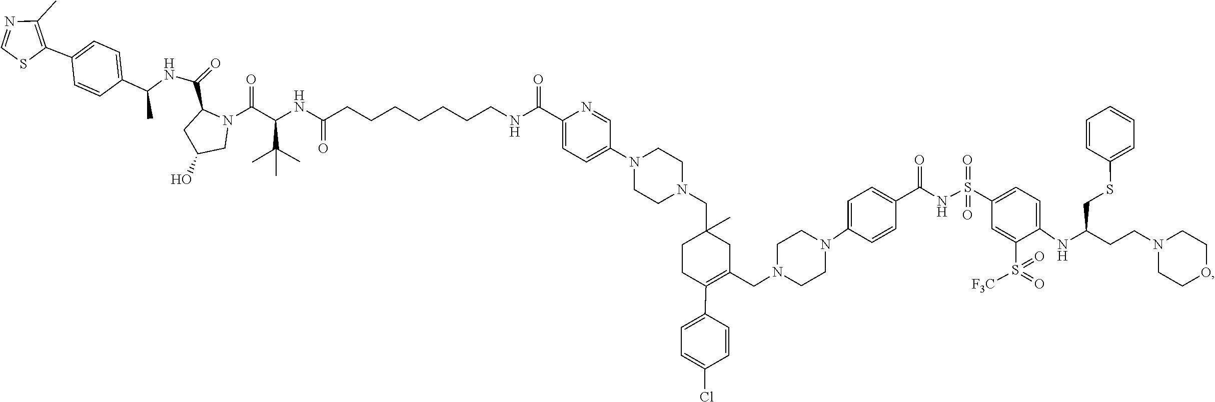 [SMILES: Cc1ncsc1-c1ccc([C@H](C)NC(=O)[C@@H]2C[C@@H](O)CN2C(=O)[C@@H](NC(=O)CCCCCCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cn2)C(C)(C)C)cc1] |
| 7 | 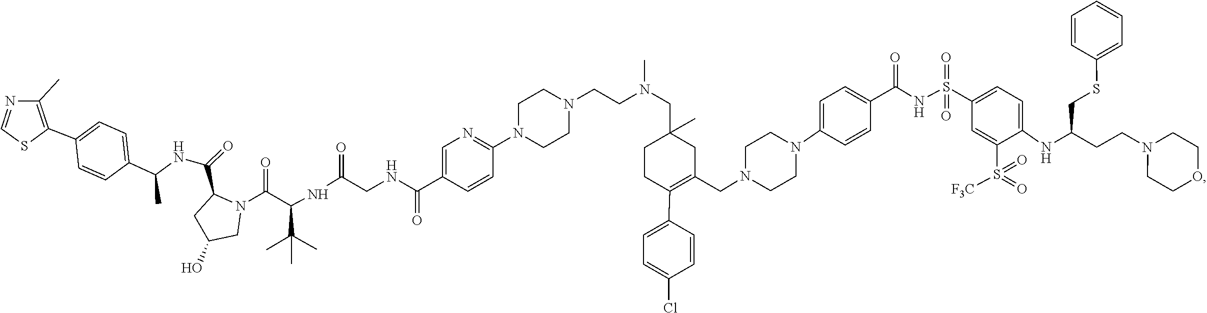 [SMILES: Cc1ncsc1-c1ccc([C@H](C)NC(=O)[C@@H]2C[C@@H](O)CN2C(=O)[C@@H](NC(=O)CNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 8 | 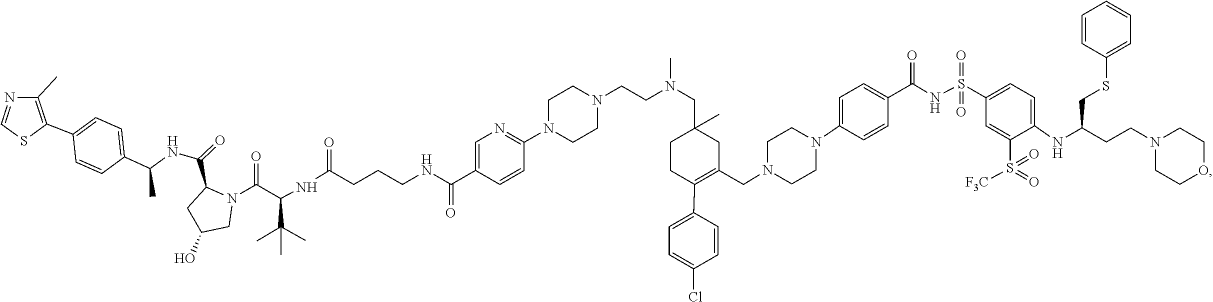 [SMILES: Cc1ncsc1-c1ccc([C@H](C)NC(=O)[C@@H]2C[C@@H](O)CN2C(=O)[C@@H](NC(=O)CCCNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 9 | 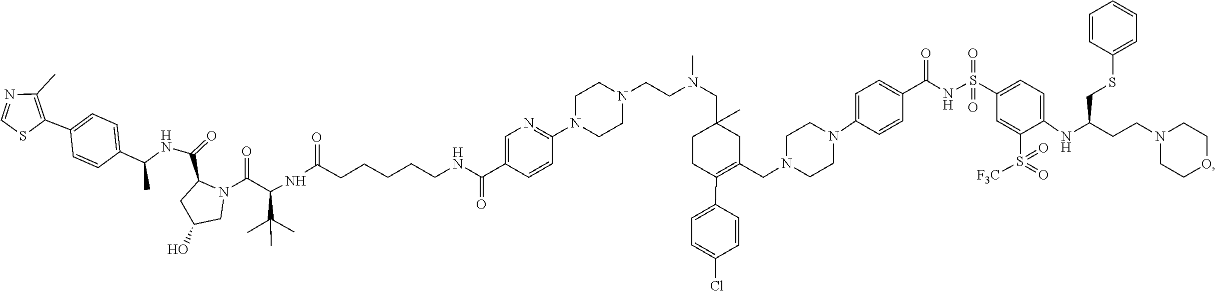 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)nc2)C(C)(C)C)cc1] |
| 10 | 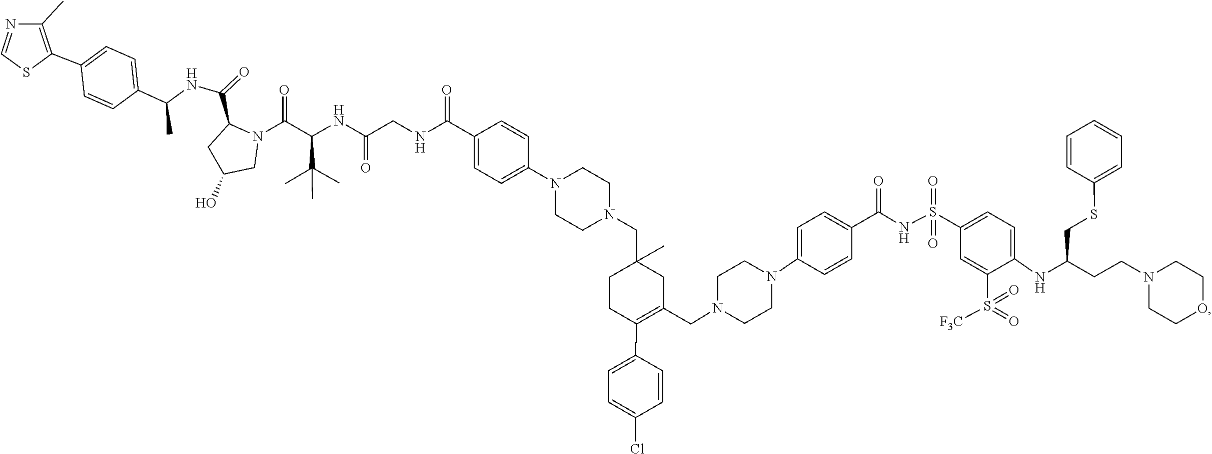 [SMILES: Cc1ncsc1-c1ccc([C@H](C)NC(=O)[C@@H]2C[C@@H](O)CN2C(=O)[C@@H](NC(=O)CNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 11 | 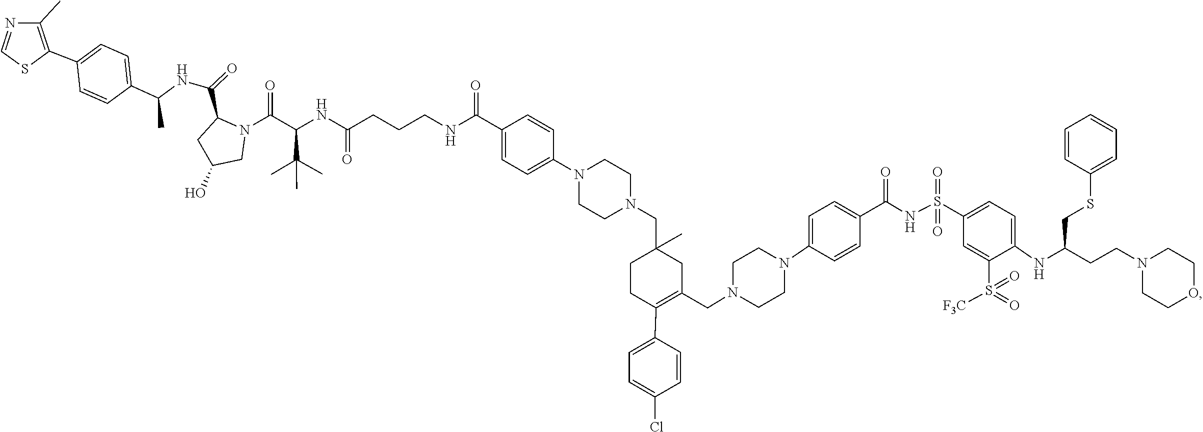 [SMILES: Cc1ncsc1-c1ccc([C@H](C)NC(=O)[C@@H]2C[C@@H](O)CN2C(=O)[C@@H](NC(=O)CCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 12 | 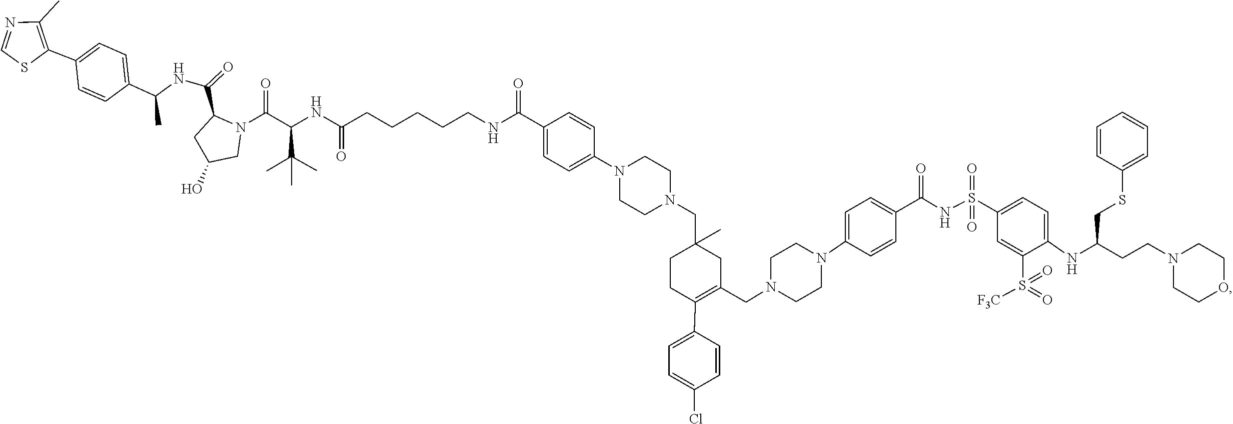 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 13 | 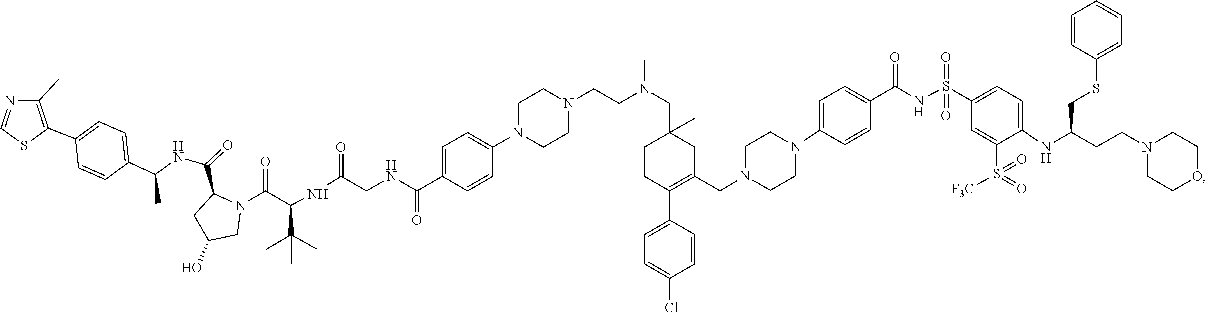 [SMILES: Cc1ncsc1-c1ccc([C@H](C)NC(=O)[C@@H]2C[C@@H](O)CN2C(=O)[C@@H](NC(=O)CNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 14 | 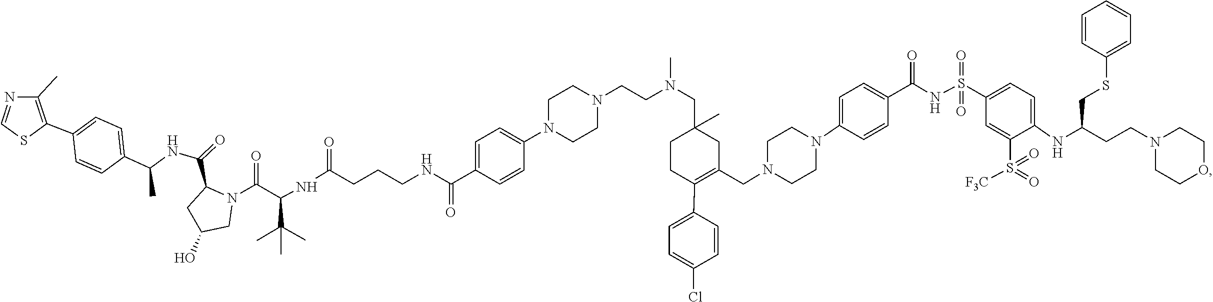 [SMILES: Cc1ncsc1-c1ccc([C@H](C)NC(=O)[C@@H]2C[C@@H](O)CN2C(=O)[C@@H](NC(=O)CCCNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 15 | 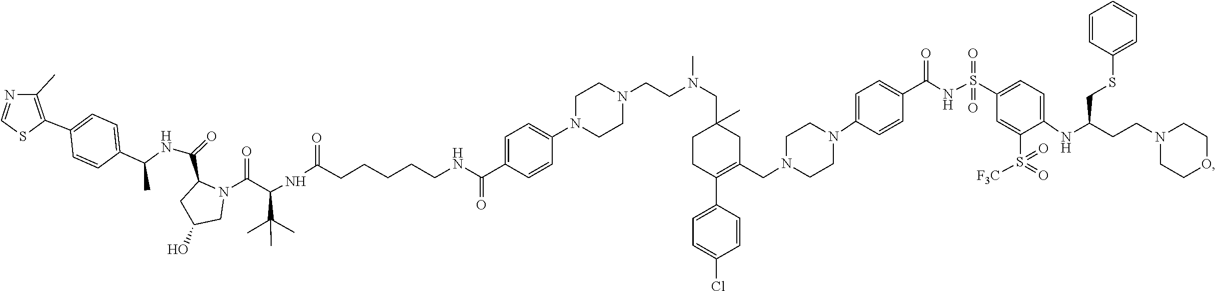 [SMILES: Cc1ncsc1-c1ccc([C@H](C)NC(=O)[C@@H]2C[C@@H](O)CN2C(=O)[C@@H](NC(=O)CCCCCNC(=O)c2ccc(N3CCN(CCN(C)CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)cc2)C(C)(C)C)cc1] |
| 16 | 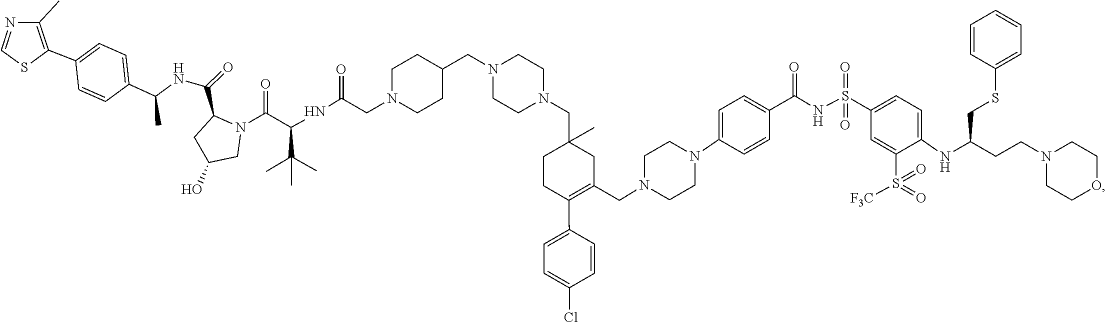 [SMILES: Cc1ncsc1-c1ccc([C@H](C)NC(=O)[C@@H]2C[C@@H](O)CN2C(=O)[C@@H](NC(=O)CN2CCC(CN3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 17 | 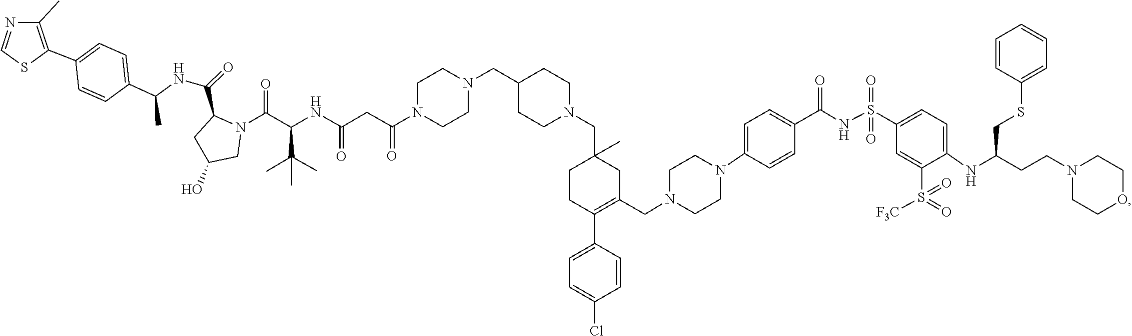 [SMILES: Cc1ncsc1-c1ccc([C@H](C)NC(=O)[C@@H]2C[C@@H](O)CN2C(=O)[C@@H](NC(=O)CC(=O)N2CCN(CC3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 18 | 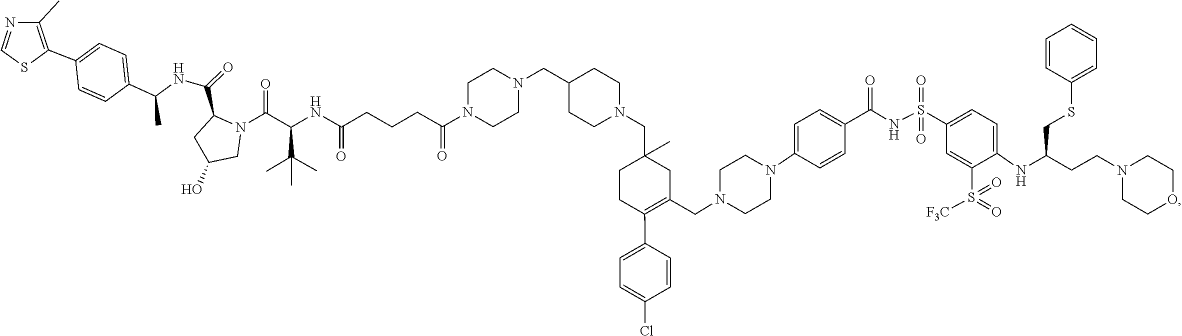 [SMILES: Cc1ncsc1-c1ccc([C@H](C)NC(=O)[C@@H]2C[C@@H](O)CN2C(=O)[C@@H](NC(=O)CCCC(=O)N2CCN(CC3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 19 | 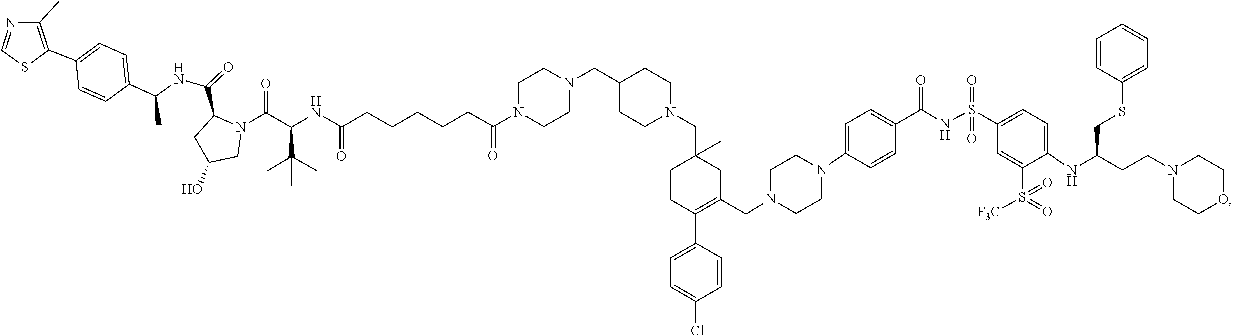 [SMILES: Cc1ncsc1-c1ccc([C@H](C)NC(=O)[C@@H]2C[C@@H](O)CN2C(=O)[C@@H](NC(=O)CCCCCC(=O)N2CCN(CC3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 20 | 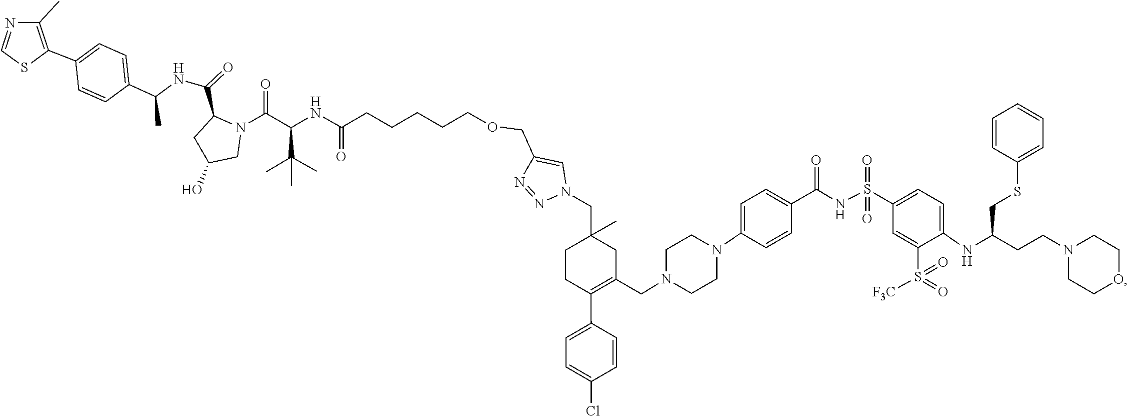 [SMILES: Cc1ncsc1-c1ccc([C@H](C)NC(=O)[C@@H]2C[C@@H](O)CN2C(=O)[C@@H](NC(=O)CCCCCOCc2cn(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1] |
| 21 | 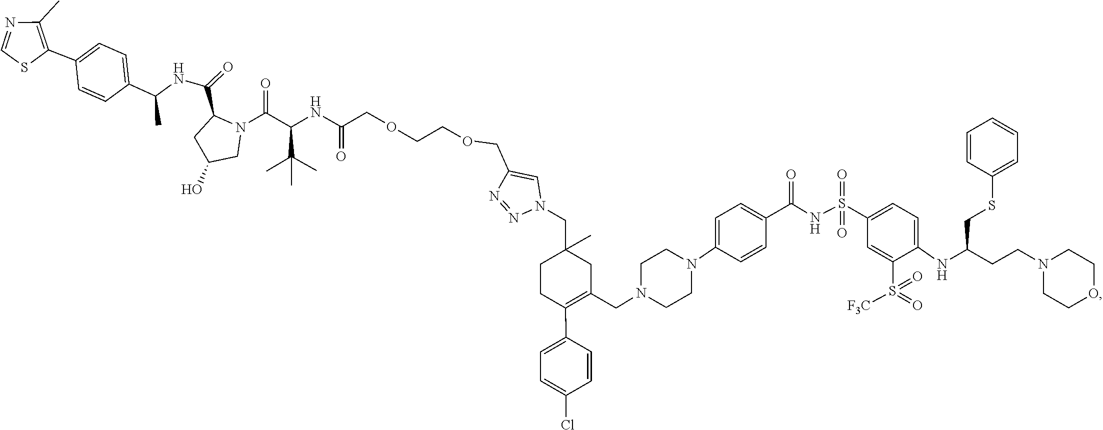 [SMILES: Cc1ncsc1-c1ccc([C@H](C)NC(=O)[C@@H]2C[C@@H](O)CN2C(=O)[C@@H](NC(=O)COCCOCc2cn(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1] |
| 22 | 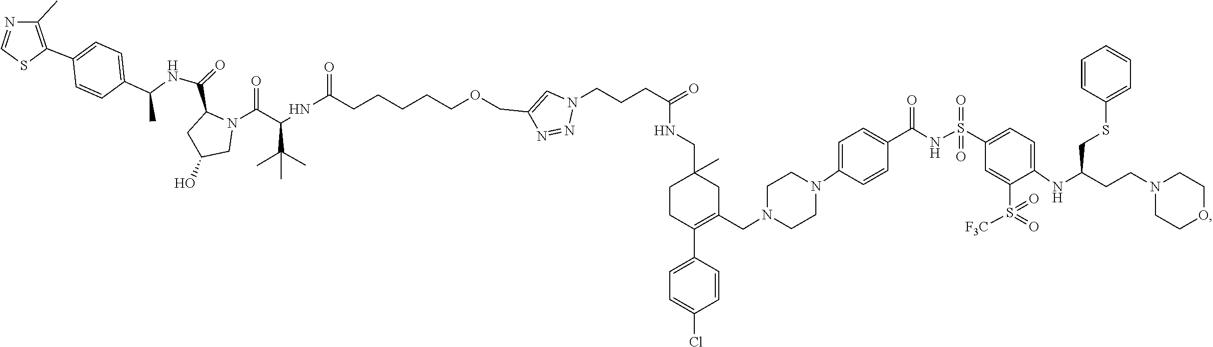 [SMILES: Cc1ncsc1-c1ccc([C@H](C)NC(=O)[C@@H]2C[C@@H](O)CN2C(=O)[C@@H](NC(=O)CCCCCOCc2cn(CCCC(=O)NCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1] |
| 23 | 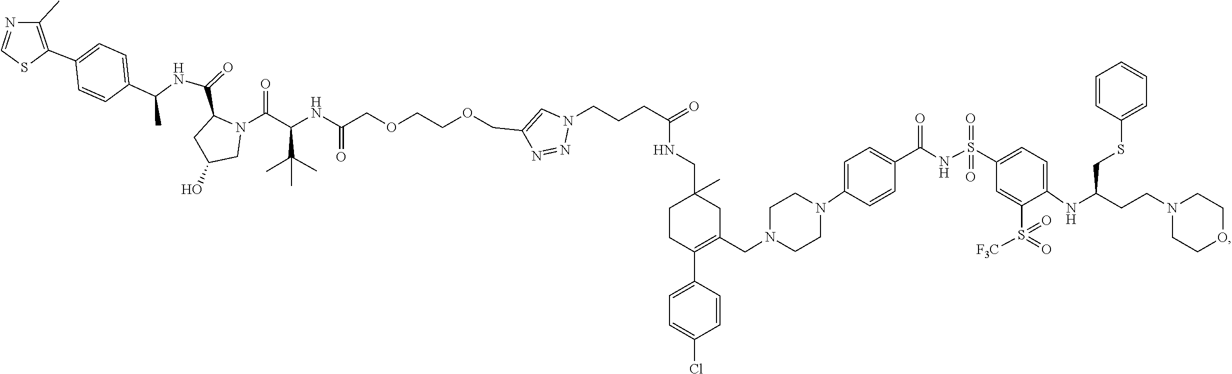 [SMILES: Cc1ncsc1-c1ccc([C@H](C)NC(=O)[C@@H]2C[C@@H](O)CN2C(=O)[C@@H](NC(=O)COCCOCc2cn(CCCC(=O)NCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1] |
| 25 | 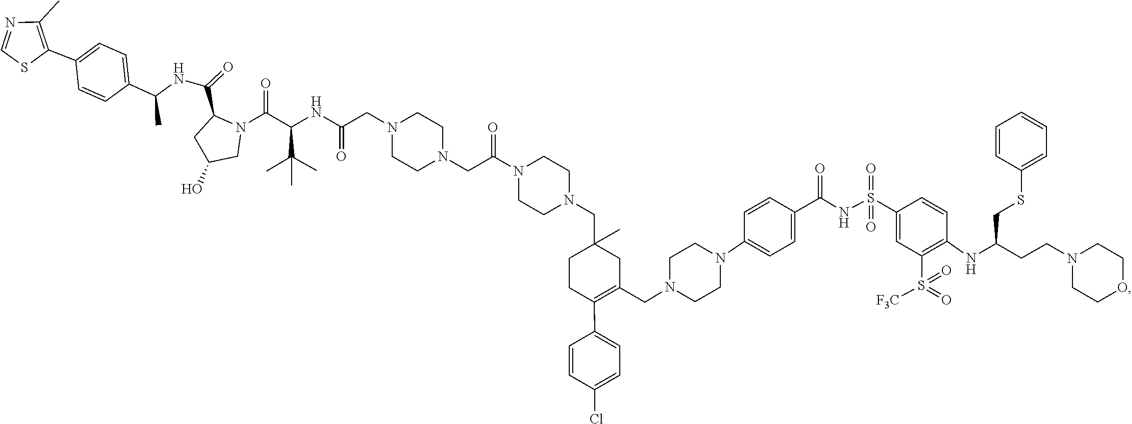 [SMILES: Cc1ncsc1-c1ccc([C@H](C)NC(=O)[C@@H]2C[C@@H](O)CN2C(=O)[C@@H](NC(=O)CN2CCN(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 26 | 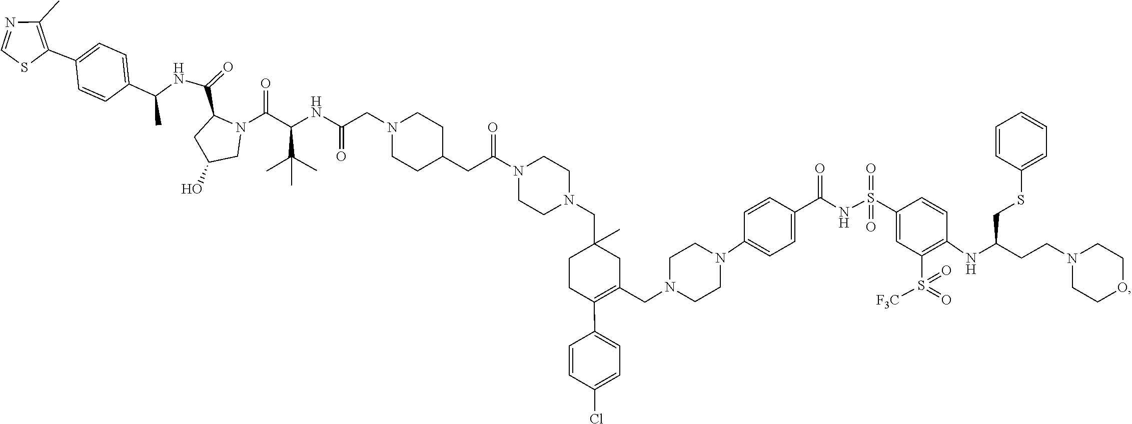 [SMILES: Cc1ncsc1-c1ccc([C@H](C)NC(=O)[C@@H]2C[C@@H](O)CN2C(=O)[C@@H](NC(=O)CN2CCC(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 27 | 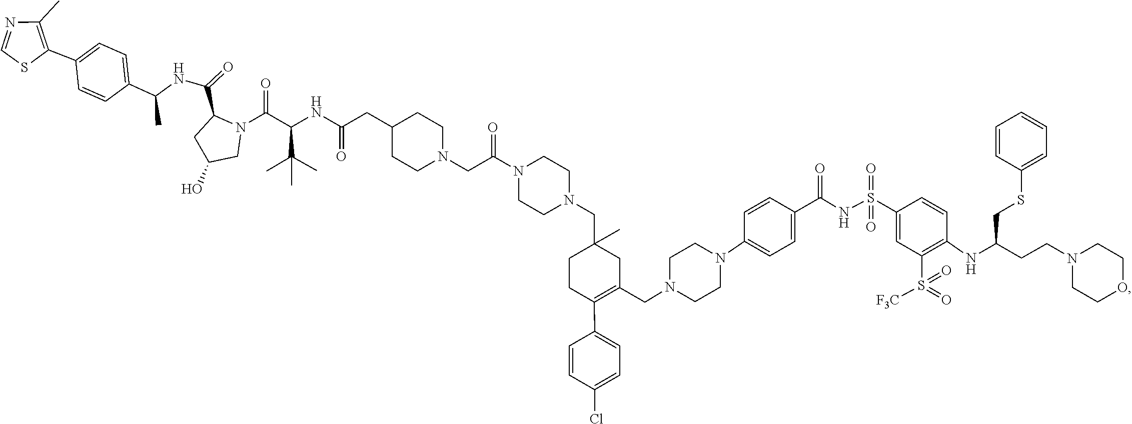 [SMILES: Cc1ncsc1-c1ccc([C@H](C)NC(=O)[C@@H]2C[C@@H](O)CN2C(=O)[C@@H](NC(=O)CC2CCN(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] |
| 28 | 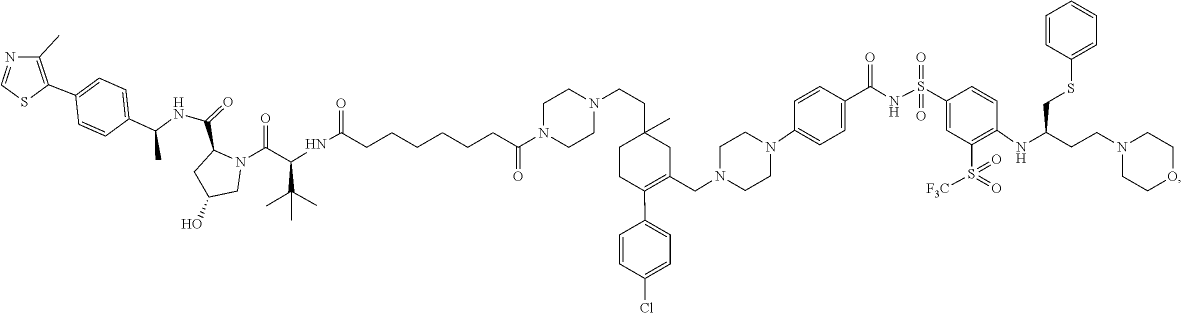 [SMILES: Cc1ncsc1-c1ccc([C@H](C)NC(=O)[C@@H]2C[C@@H](O)CN2C(=O)[C@@H](NC(=O)CCCCCCC(=O)N2CCN(CCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| 29 | 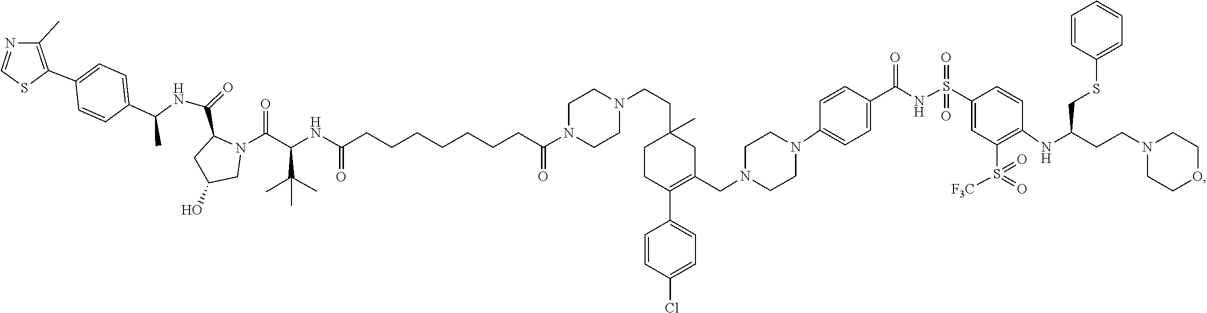 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| 30 | 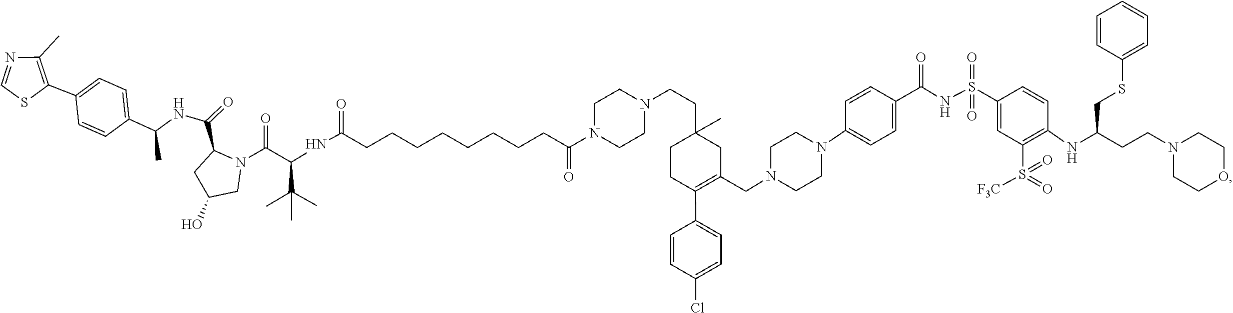 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| 31 | 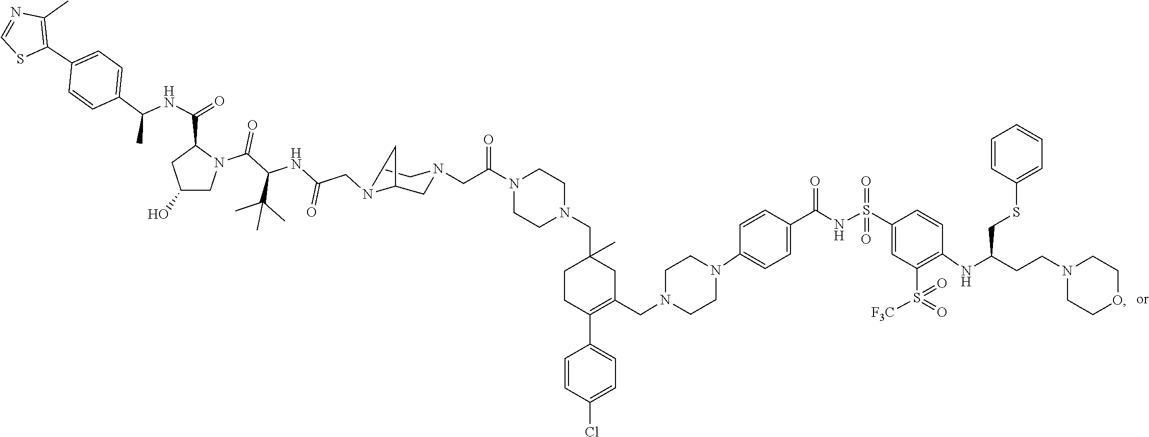 [SMILES: CCN(CC(=O)N1CCN(CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)CC1)CC1CN1CC(=O)N[C@H](C(=O)N1C[C@H](O)C[C@H]1C(=O)N[C@@H](C)c1ccc(-c2scnc2C)cc1)C(C)(C)C ; "or"] |
| 33 | 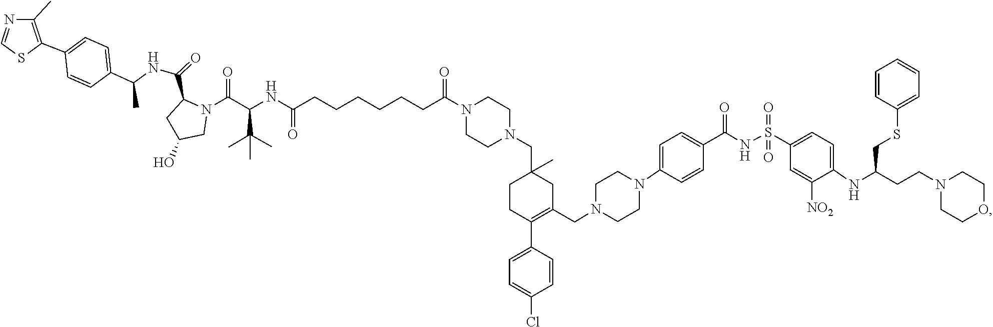 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c([N+](=O)[O-])c6)cc5)CC4)C3)CC2)C(C)(C)C)cc1] |

or a pharmaceutically acceptable salt, hydrate, solvate, or prodrug thereof.

In another aspect, the compound of Formula (II) is:

| Compound | |
|---|---|
| # | Formula |
| 32 | 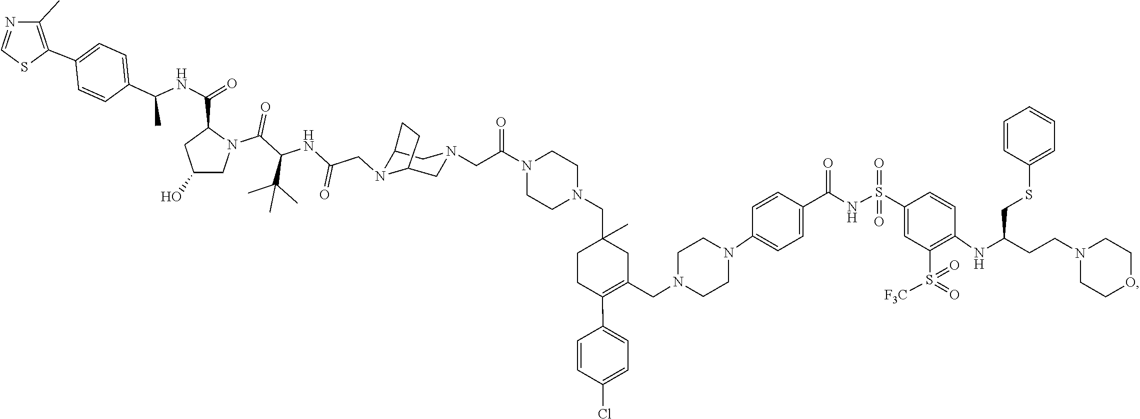 [SMILES: Cc1ncsc1-c1ccc([C@H](C)NC(=O)[C@@H]2C[C@@H](O)CN2C(=O)[C@@H](NC(=O)CN2C3CCC2CN(CC(=O)N2CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC2)C3)C(C)(C)C)cc1] |

or a pharmaceutically acceptable salt, hydrate, solvate, or prodrug thereof.

In another aspect, the compound is a compound, or salt thereof, of any of the formulae herein (e.g., Formula (I) or Formula (II)), wherein the compound is not a compound disclosed in International Patent Application Publication Number WO 2020/163823A8, or a salt thereof. In another aspect, the compound is a compound, or salt thereof, of any of the formulae herein (e.g., Formula (I) or Formula (II)), wherein the compound is not a compound provided in Table 1, or a salt thereof.

TABLE 1

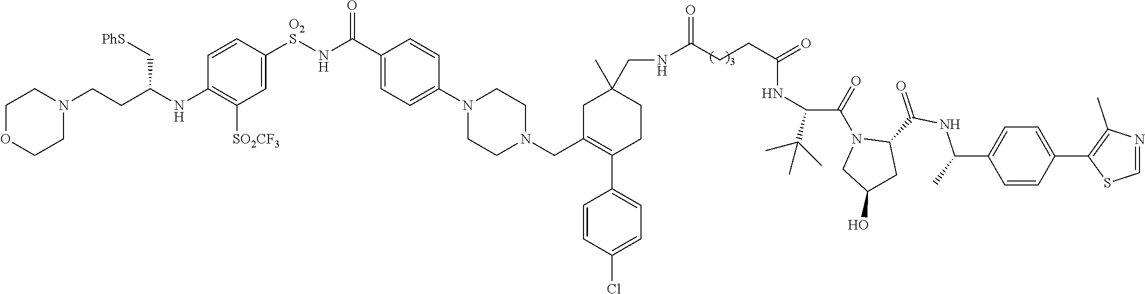

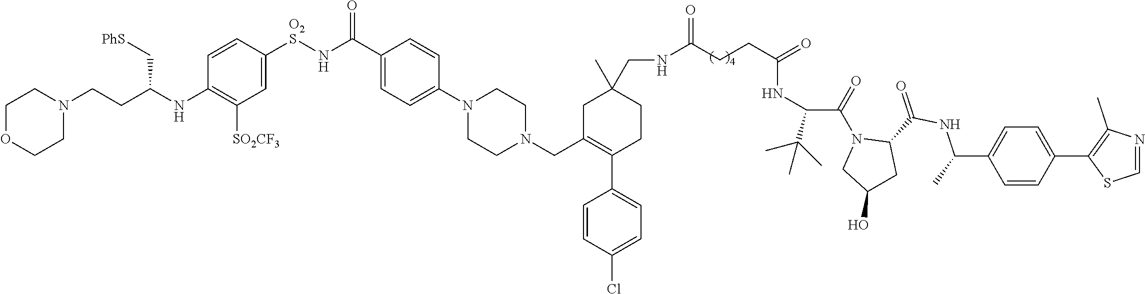

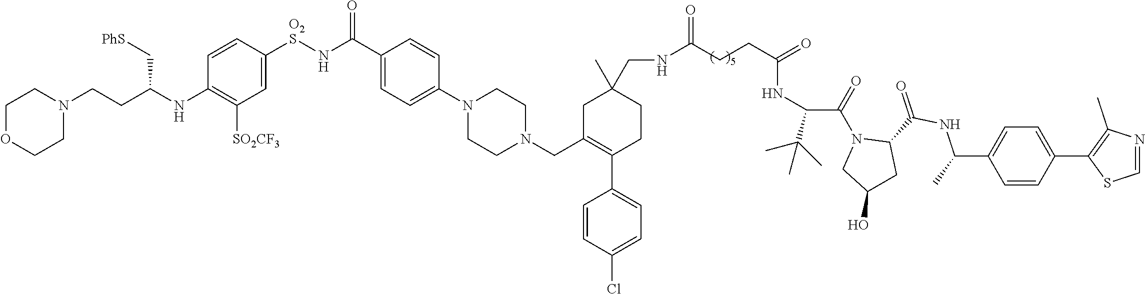

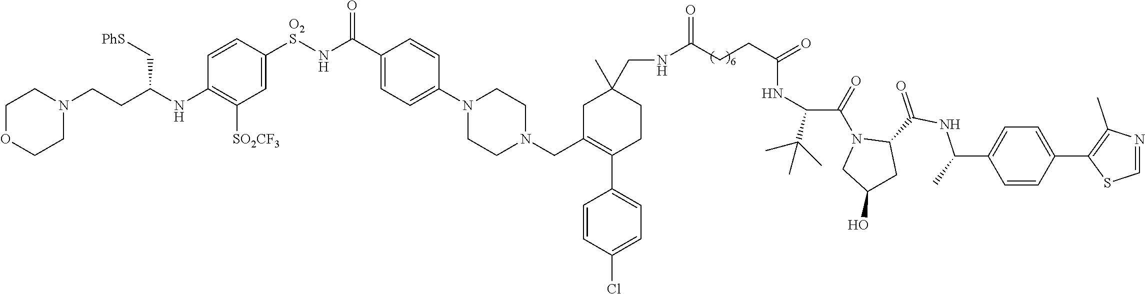

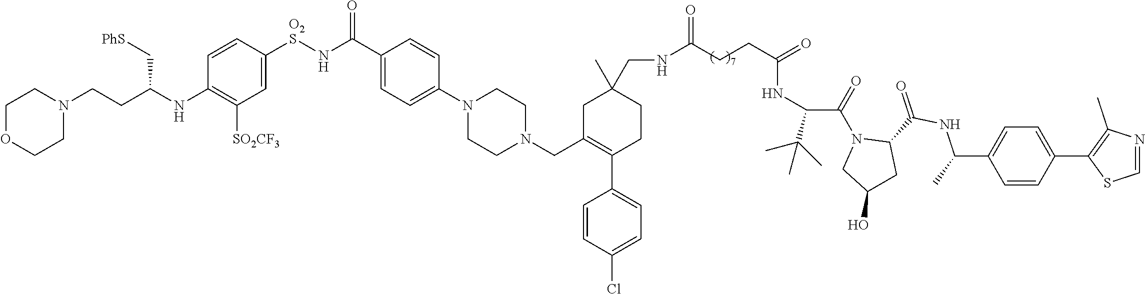

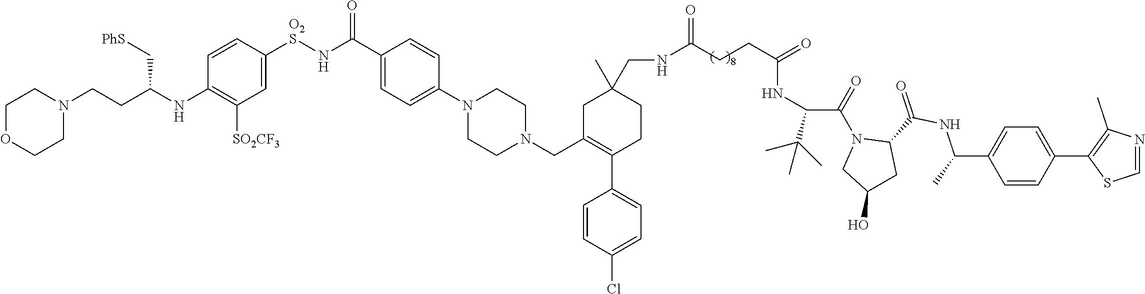

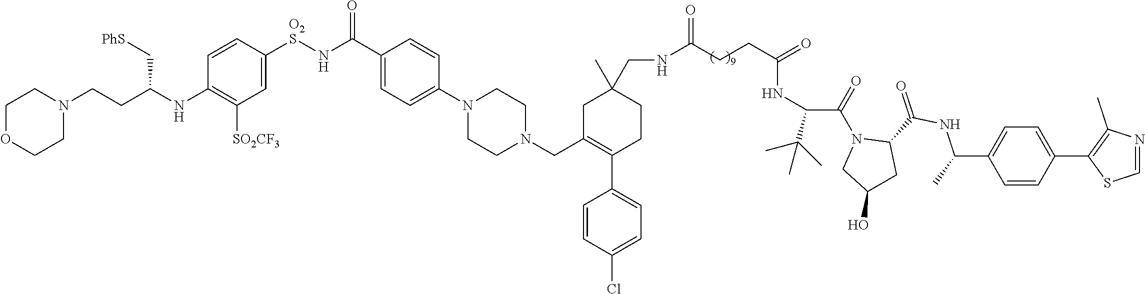

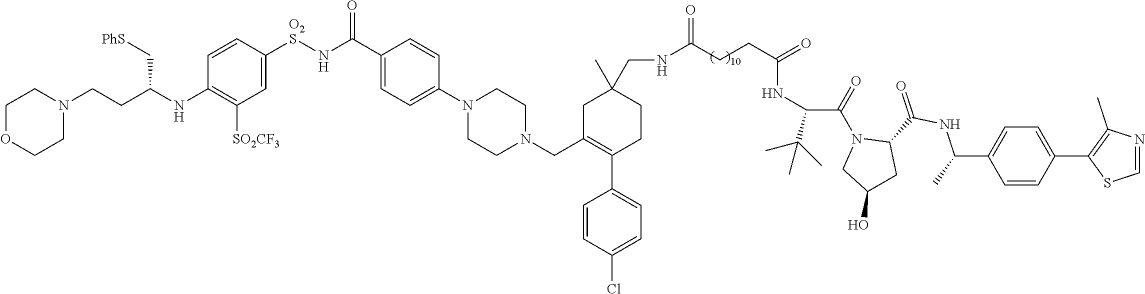

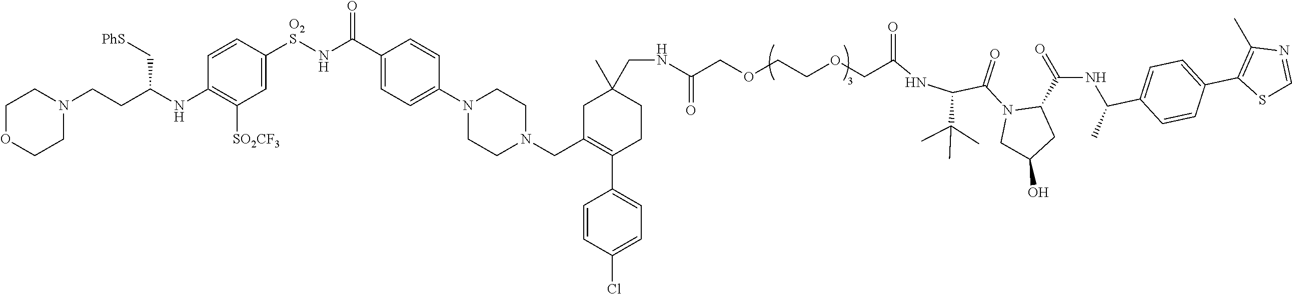

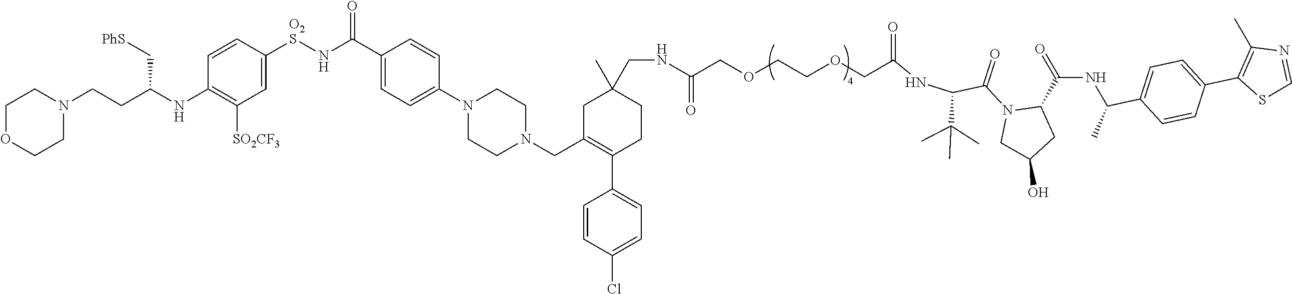

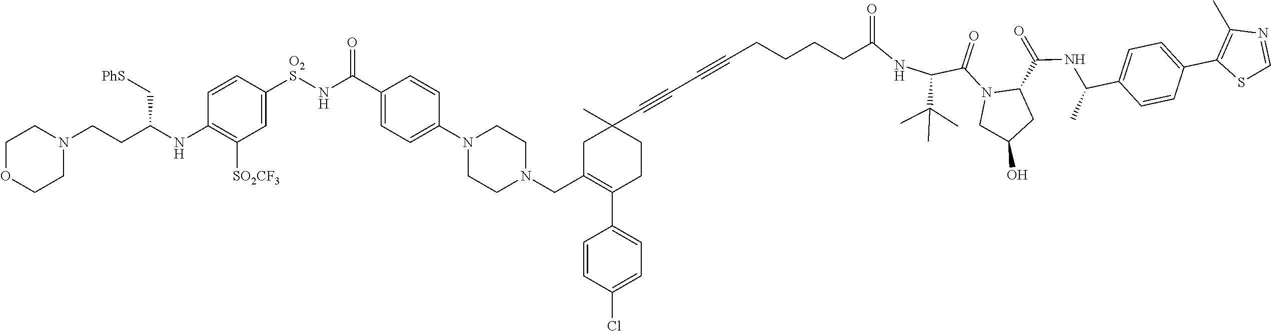

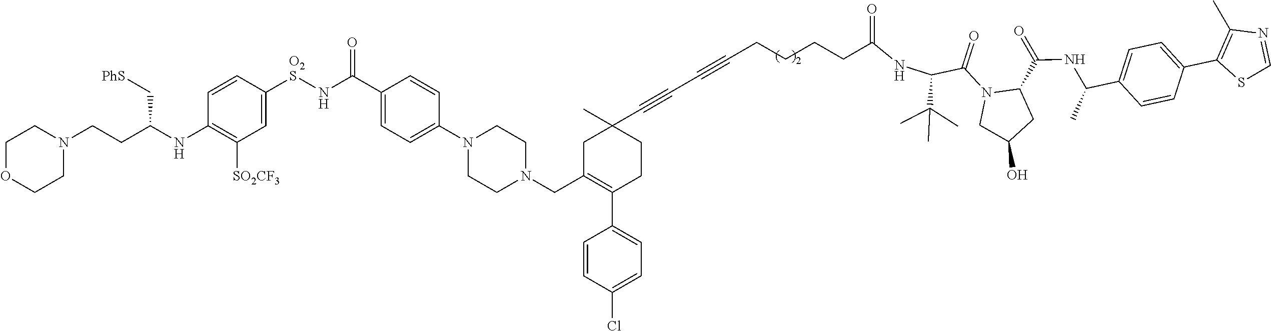

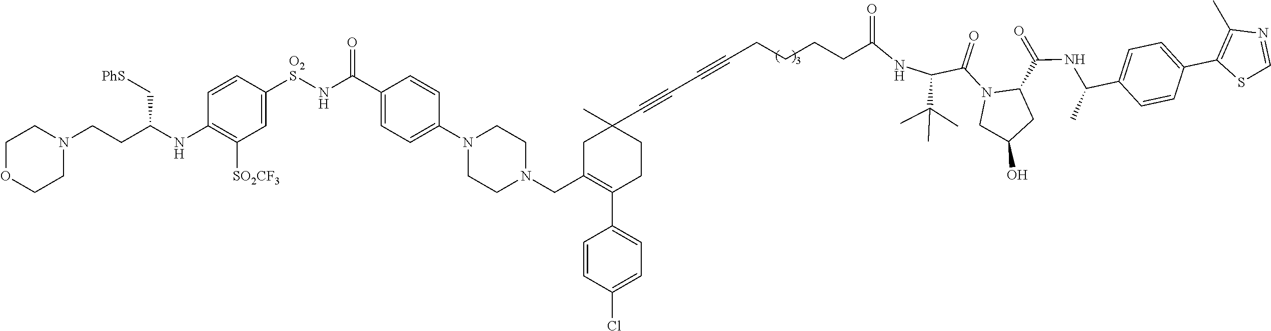

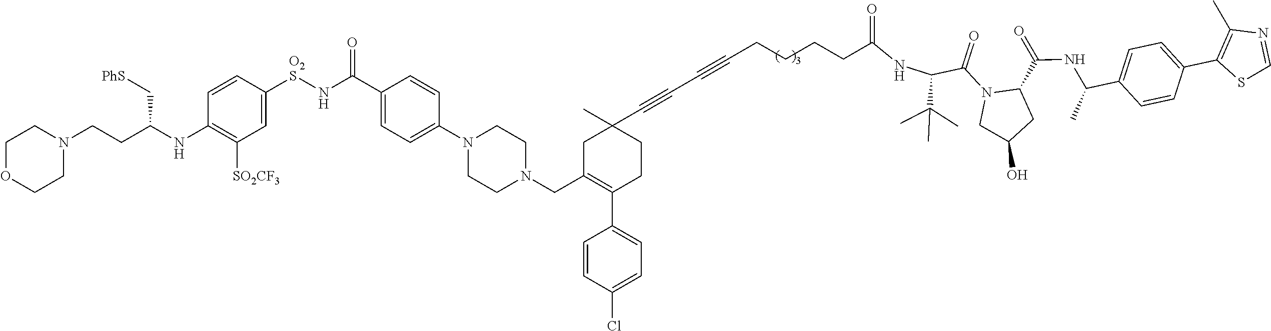

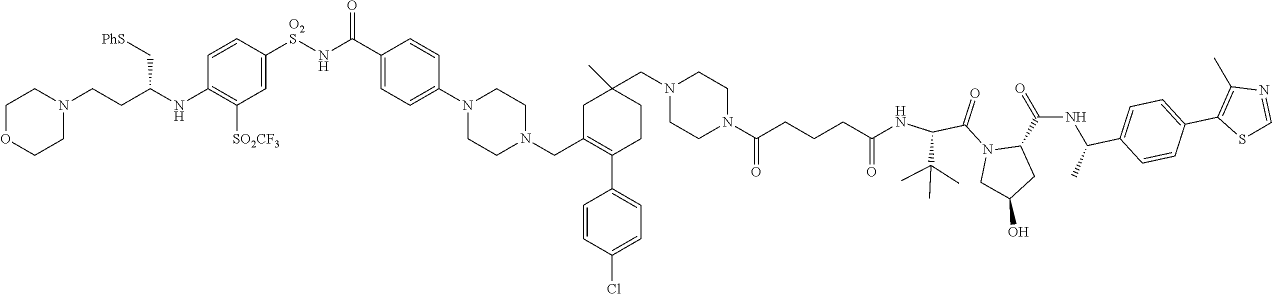

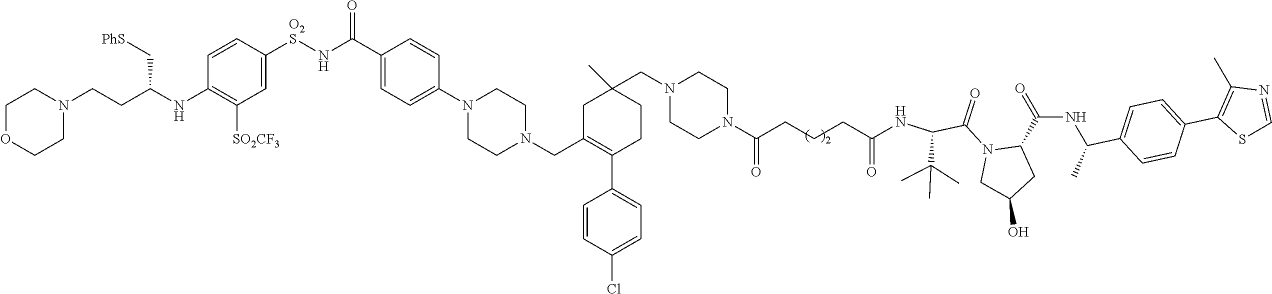

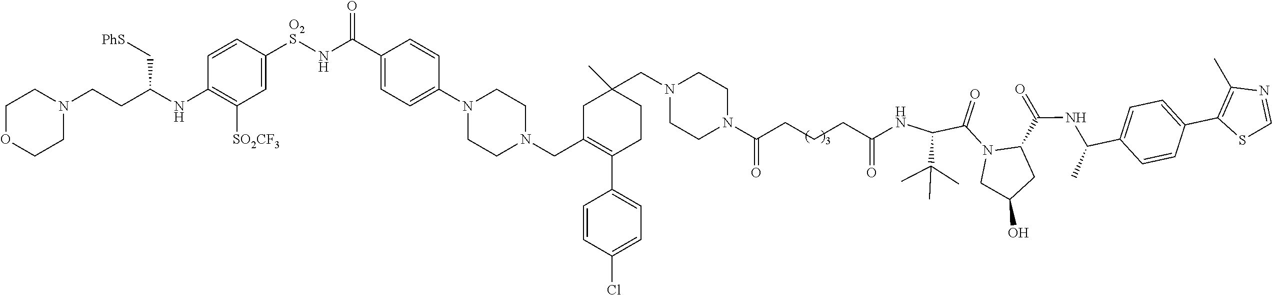

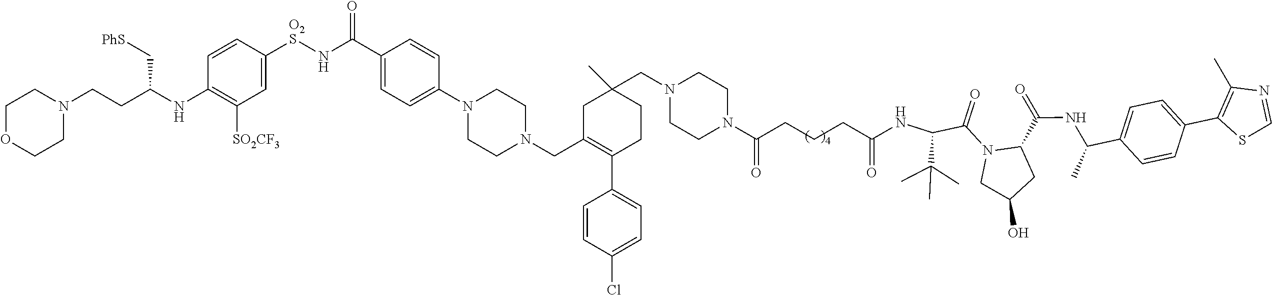

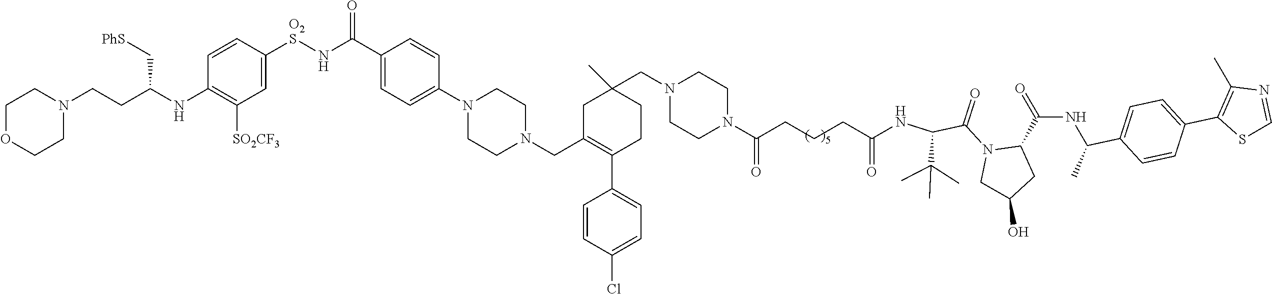

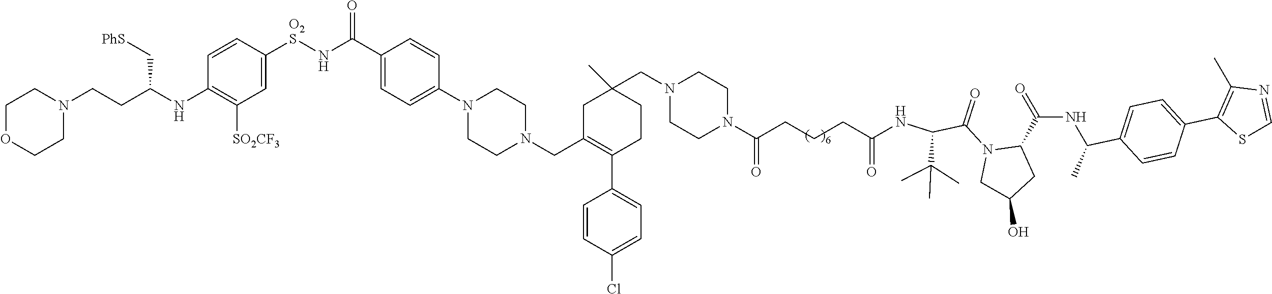

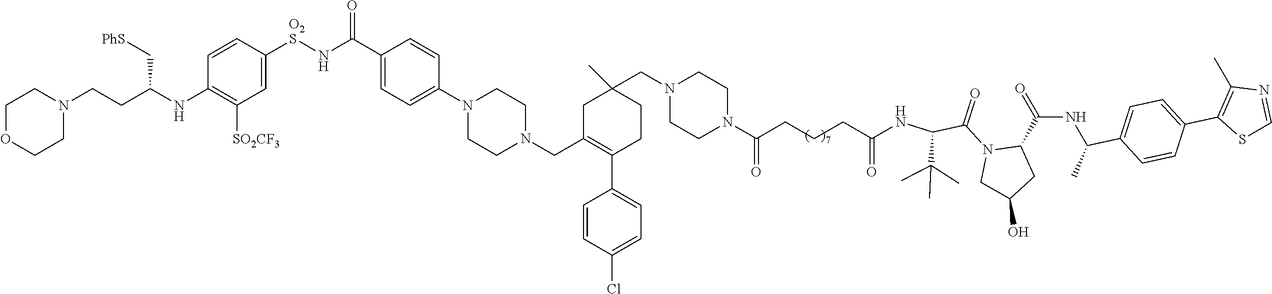

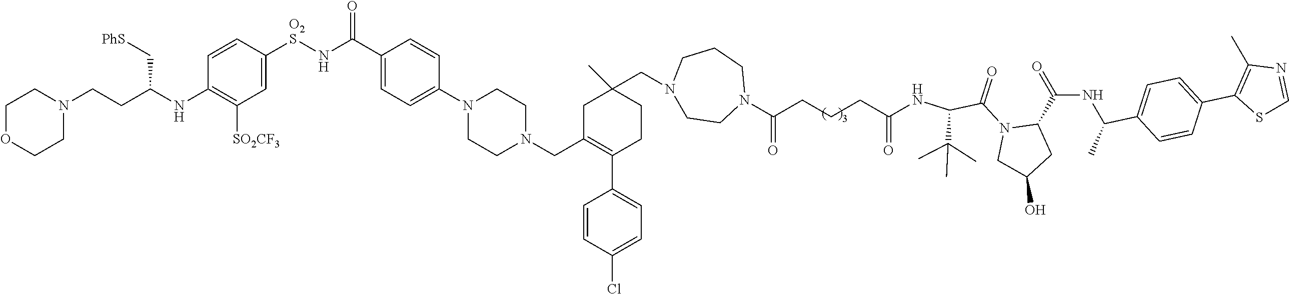

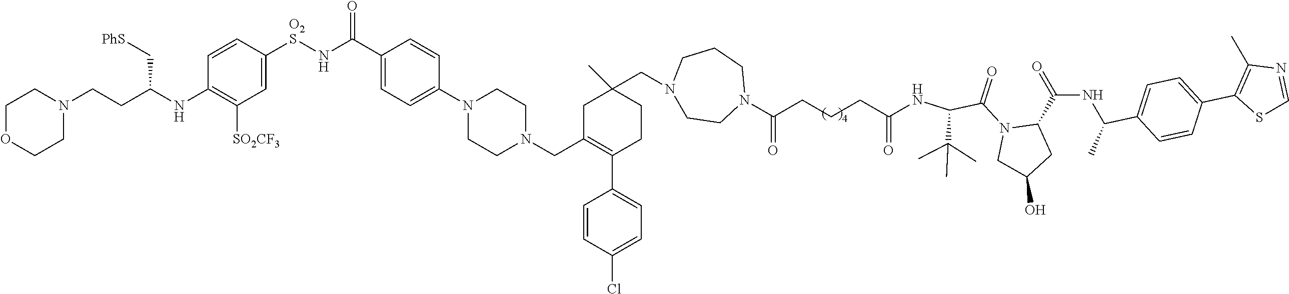

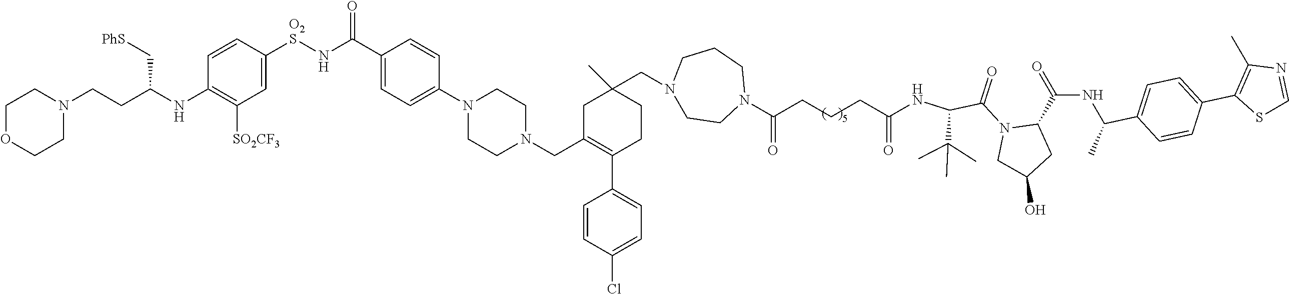

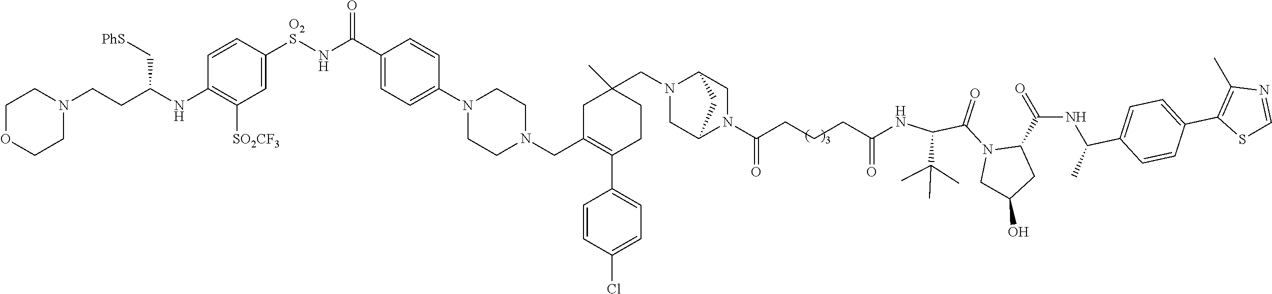

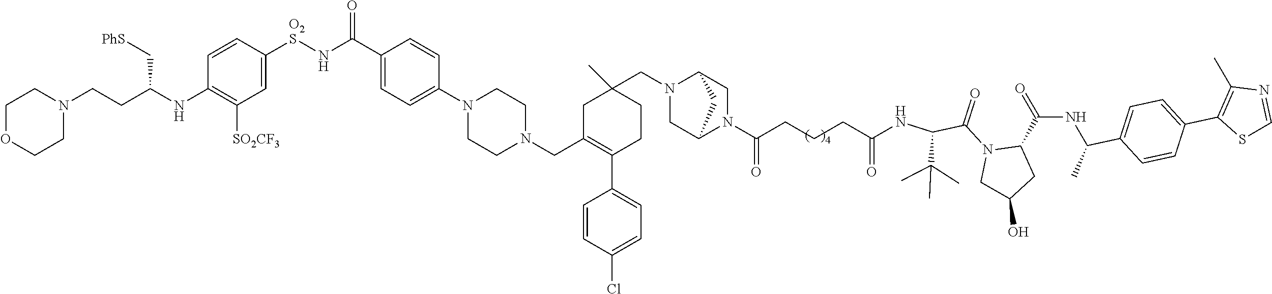

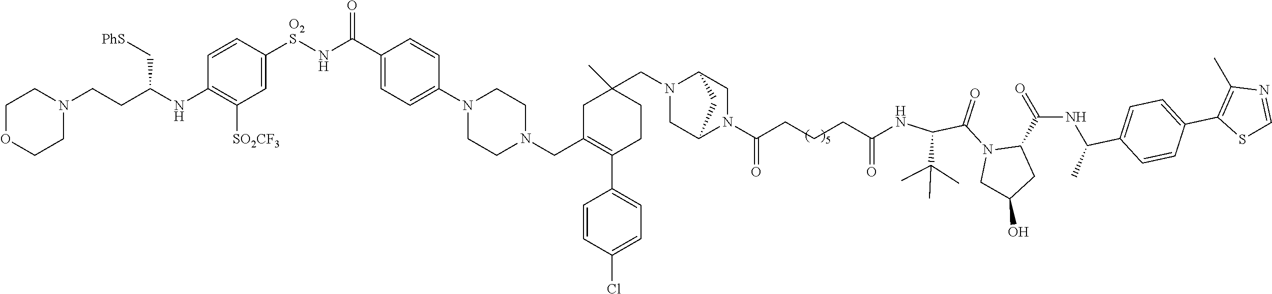

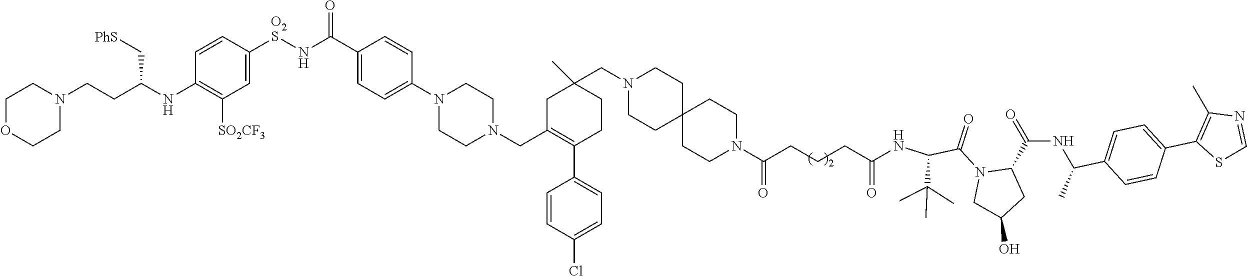

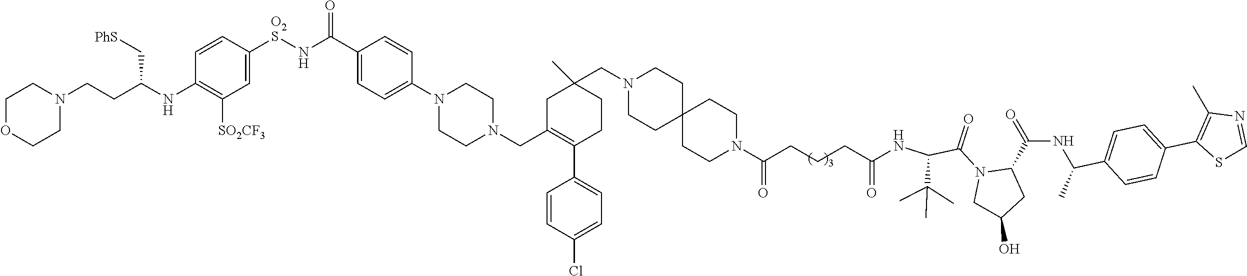

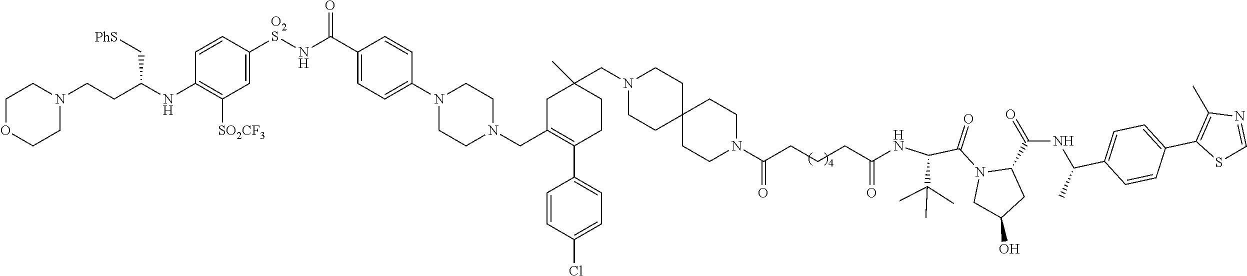

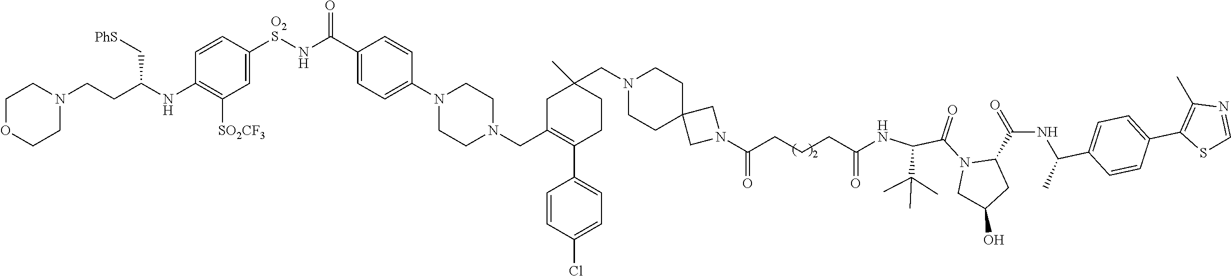

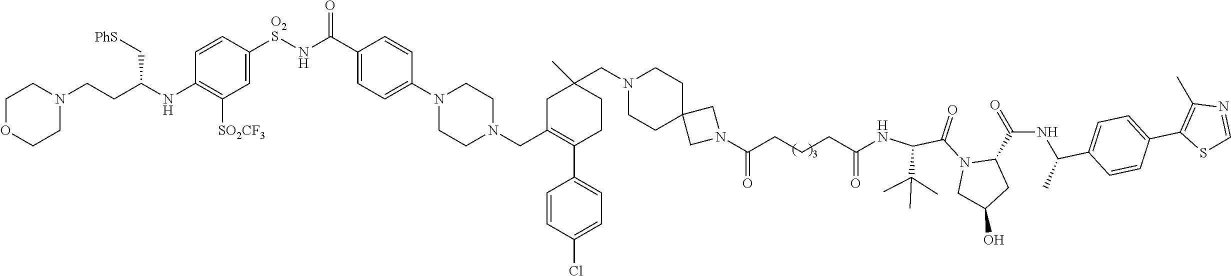

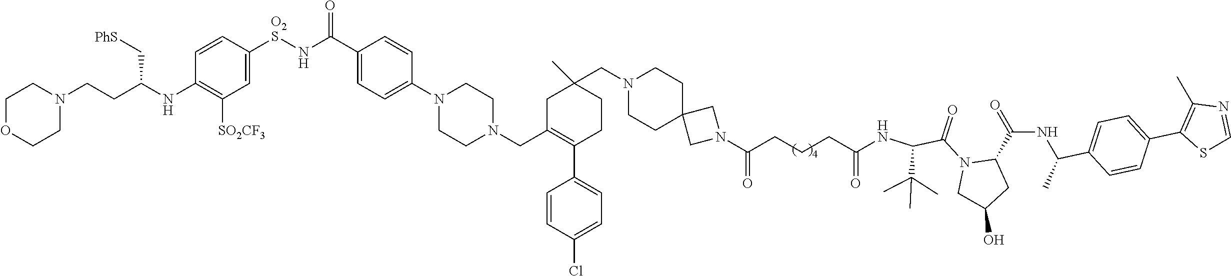

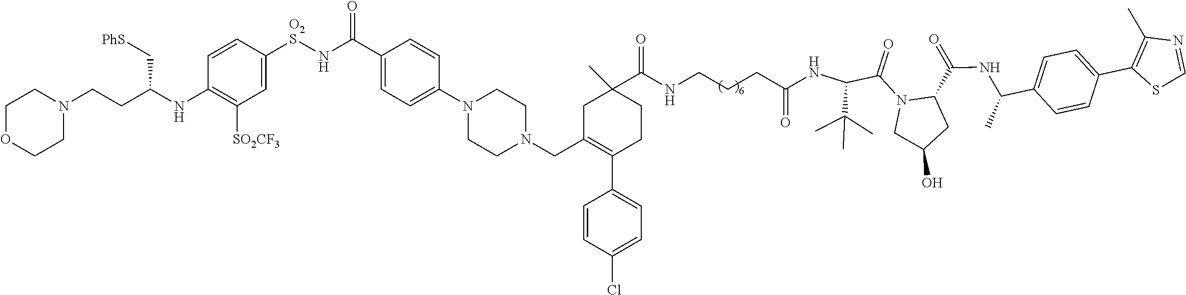

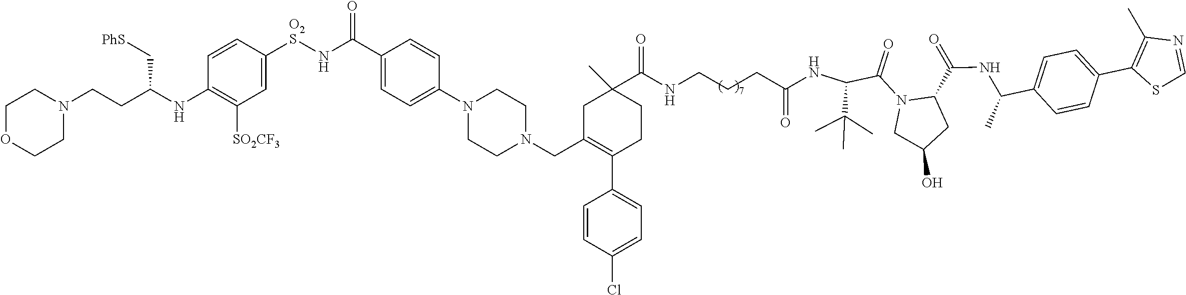

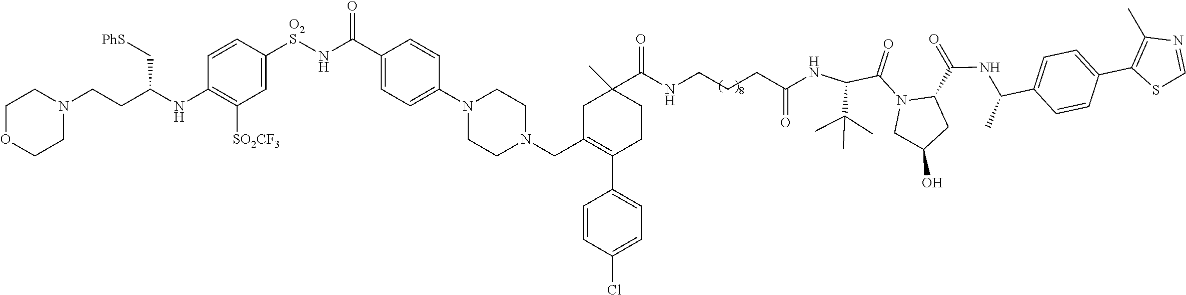

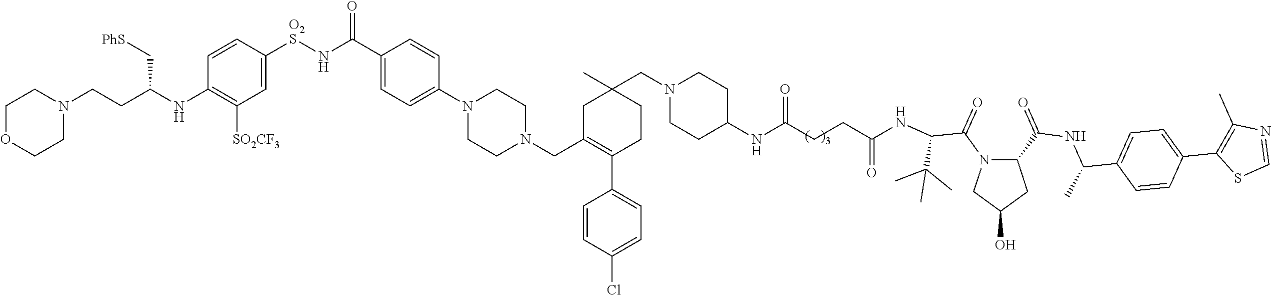

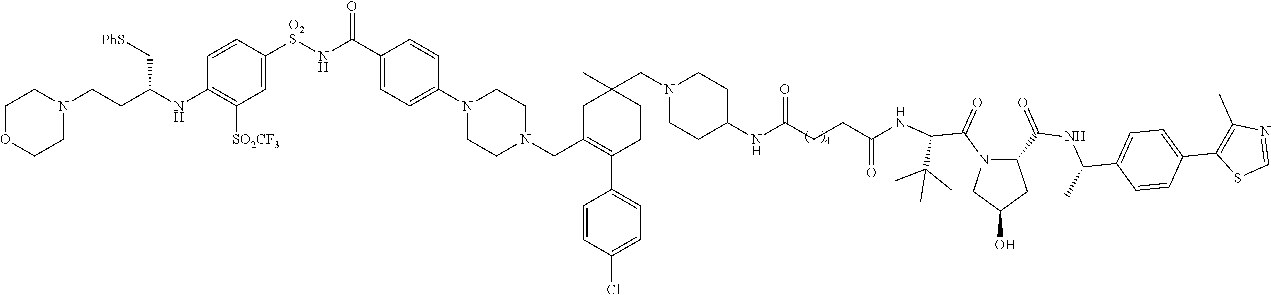

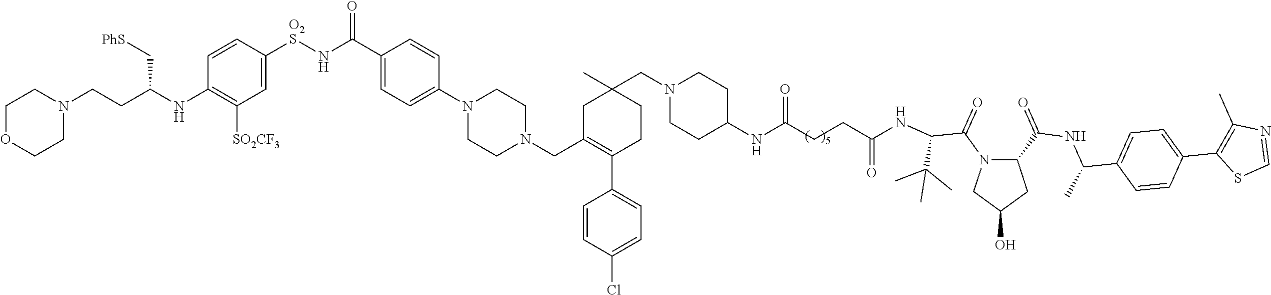

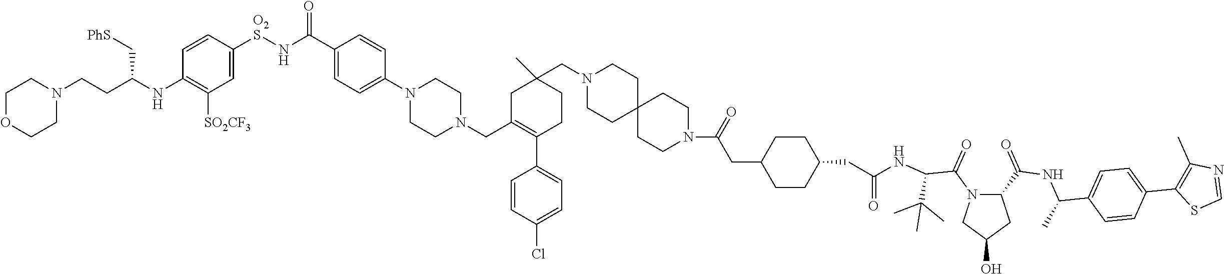

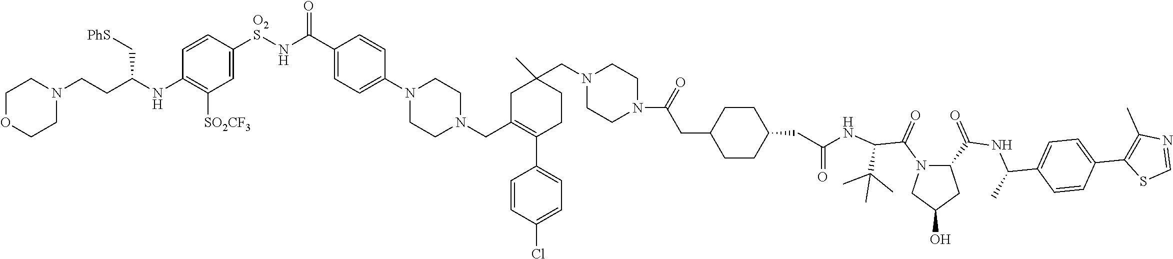

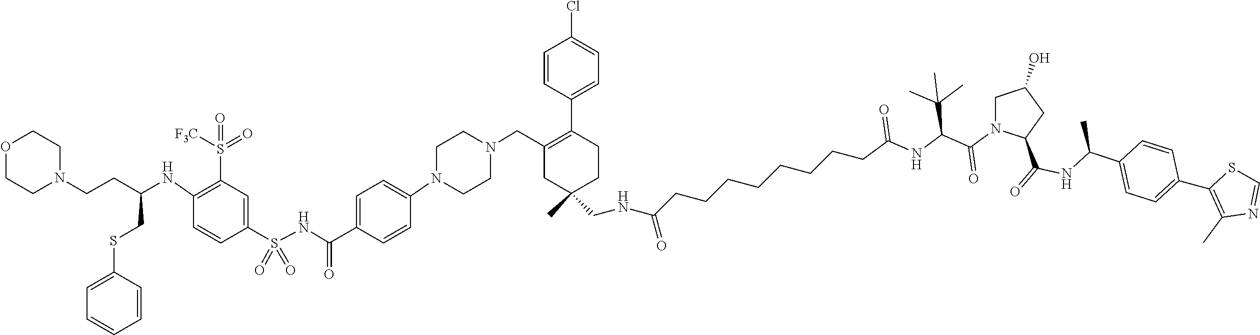 [SMILES: Cc1ncsc1-c1ccc([C@H](C)NC(=O)[C@@H]2C[C@@H](O)CN2C(=O)[C@@H](NC(=O)CCCCCCCCC(=O)NC[C@@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

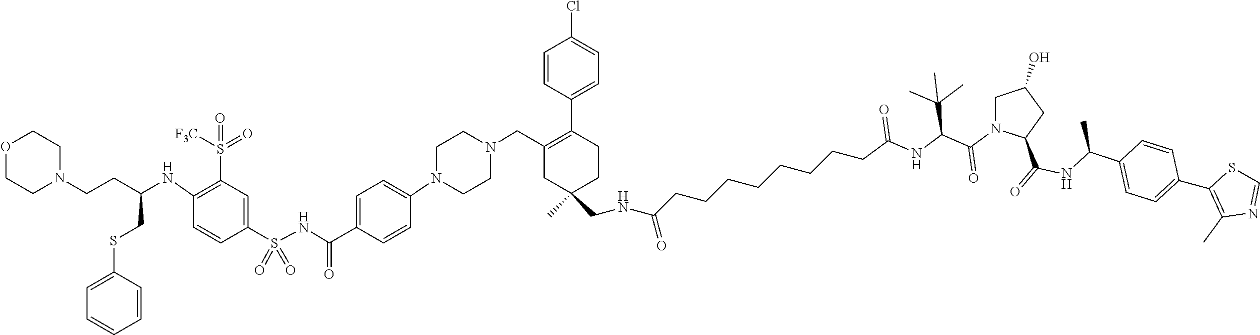 [SMILES: Cc1ncsc1-c1ccc([C@H](C)NC(=O)[C@@H]2C[C@@H](O)CN2C(=O)[C@@H](NC(=O)CCCCCCCCC(=O)NC[C@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

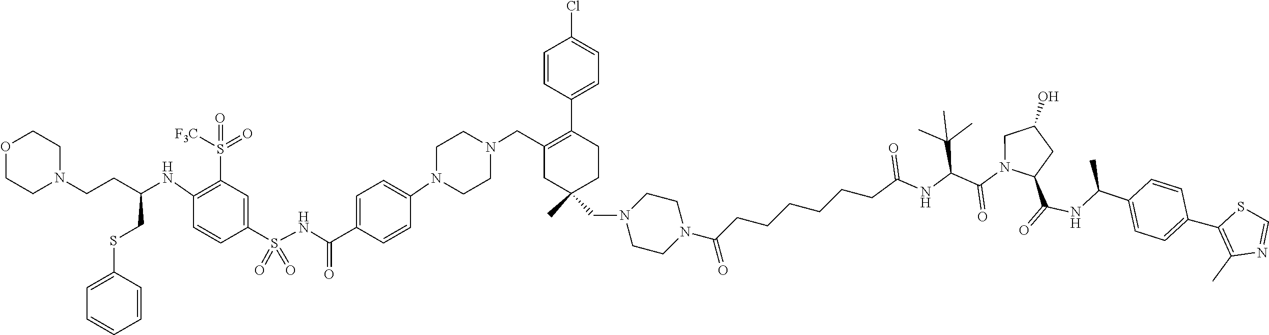 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

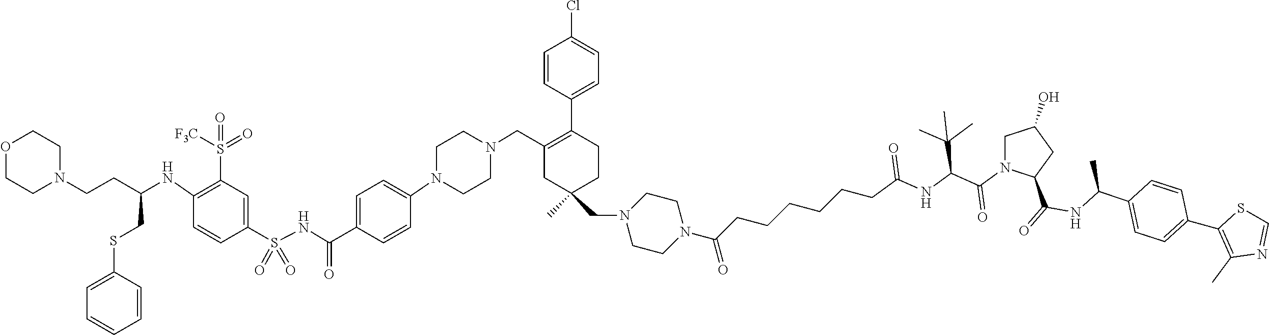 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

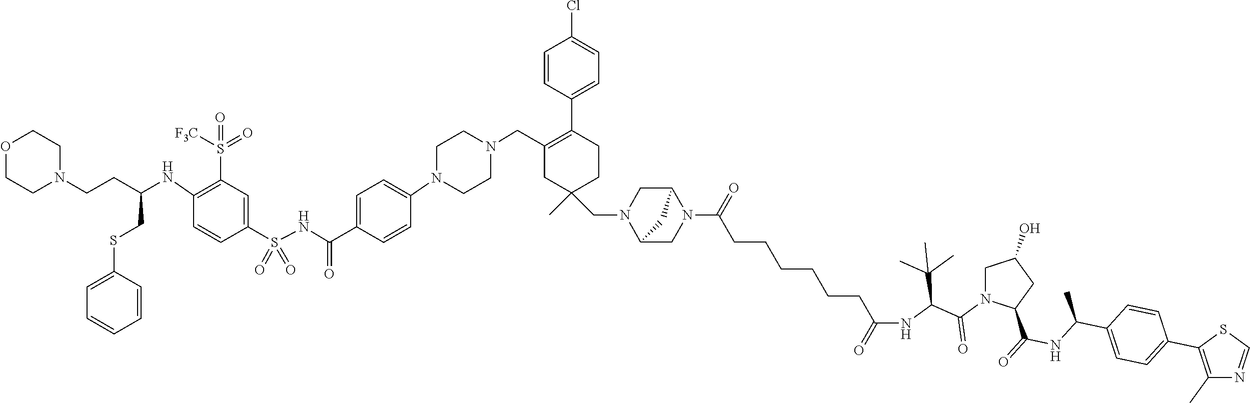 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

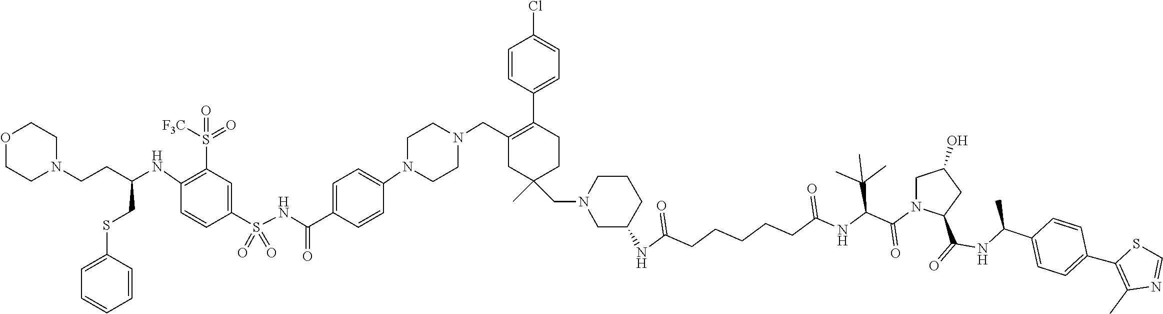 [SMILES: Cc1ncsc1-c1ccc([C@H](C)NC(=O)[C@@H]2C[C@@H](O)CN2C(=O)[C@@H](NC(=O)CCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

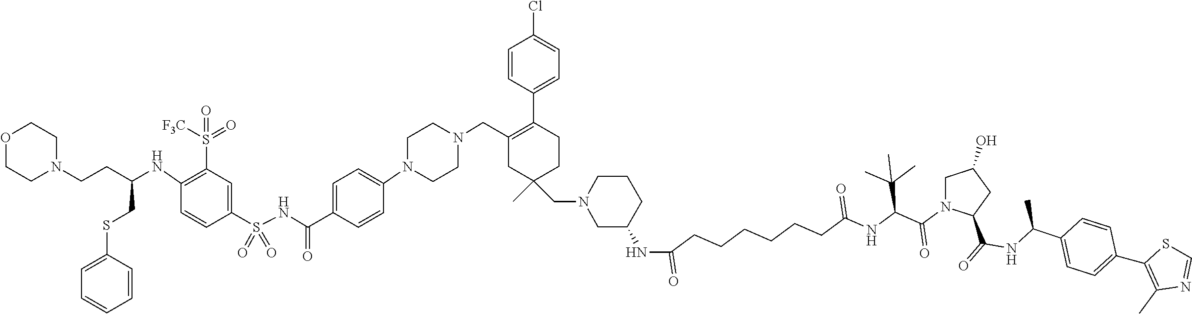 [SMILES: Cc1ncsc1-c1ccc([C@H](C)NC(=O)[C@@H]2C[C@@H](O)CN2C(=O)[C@@H](NC(=O)CCCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

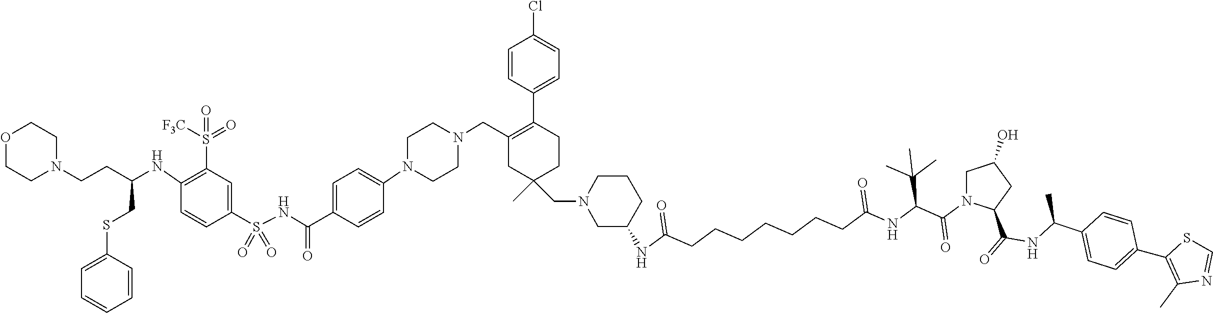 [SMILES: Cc1ncsc1-c1ccc([C@H](C)NC(=O)[C@@H]2C[C@@H](O)CN2C(=O)[C@@H](NC(=O)CCCCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

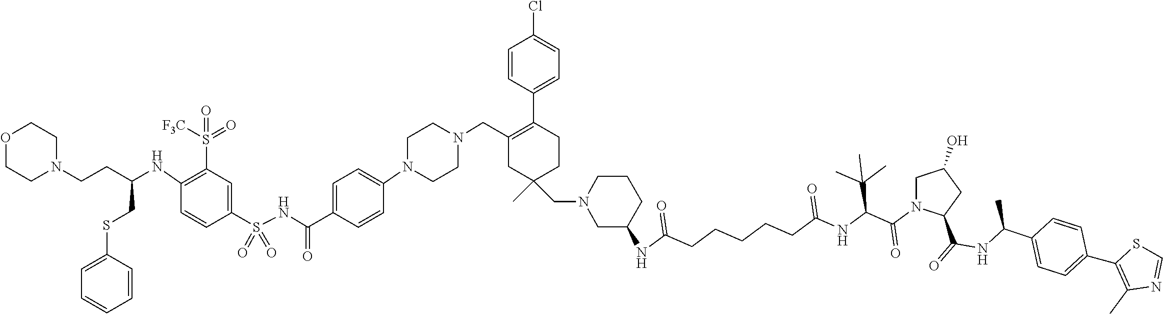 [SMILES: Cc1ncsc1-c1ccc([C@H](C)NC(=O)[C@@H]2C[C@@H](O)CN2C(=O)[C@@H](NC(=O)CCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

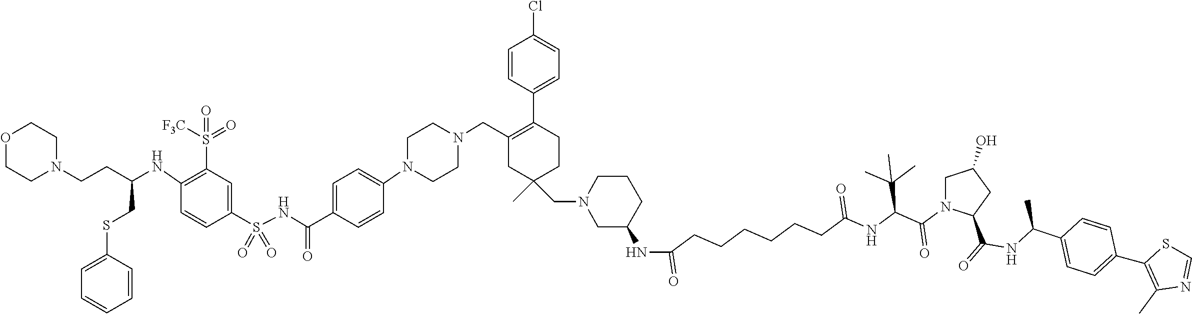 [SMILES: Cc1ncsc1-c1ccc([C@H](C)NC(=O)[C@@H]2C[C@@H](O)CN2C(=O)[C@@H](NC(=O)CCCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

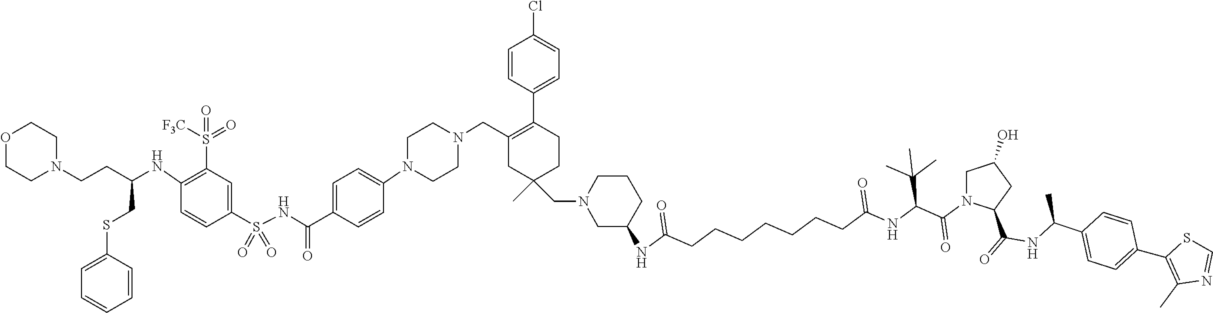 [SMILES: Cc1ncsc1-c1ccc([C@H](C)NC(=O)[C@@H]2C[C@@H](O)CN2C(=O)[C@@H](NC(=O)CCCCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1]

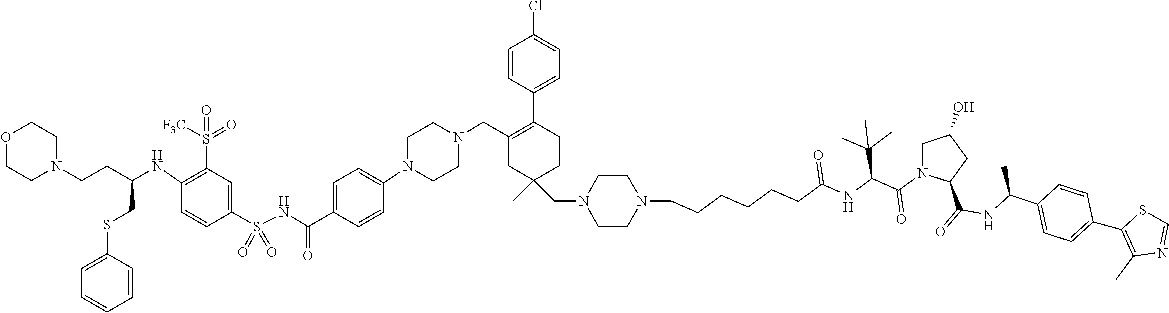 [SMILES: Cc1ncsc1-c1ccc([C@H](C)NC(=O)[C@@H]2C[C@@H](O)CN2C(=O)[C@@H](NC(=O)CCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

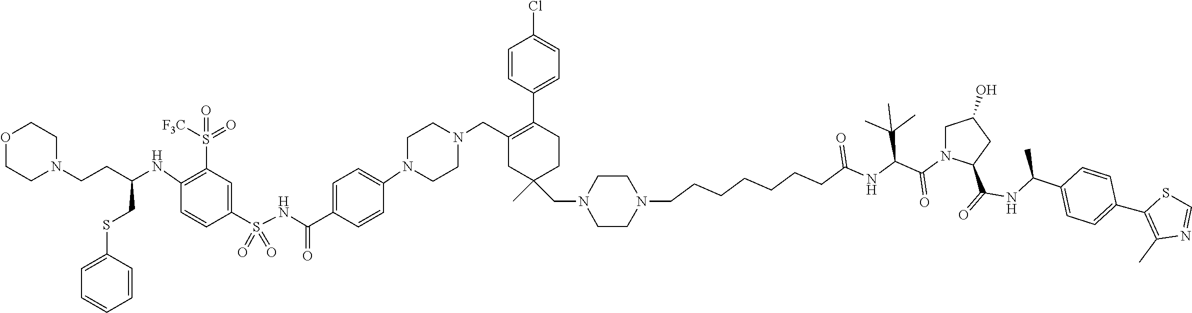 [SMILES: Cc1ncsc1-c1ccc([C@H](C)NC(=O)[C@@H]2C[C@@H](O)CN2C(=O)[C@@H](NC(=O)CCCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

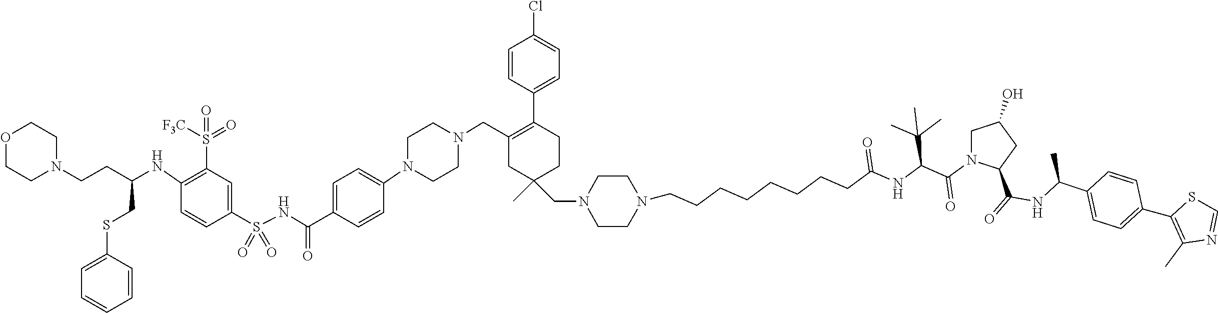 [SMILES: Cc1ncsc1-c1ccc([C@H](C)NC(=O)[C@@H]2C[C@@H](O)CN2C(=O)[C@@H](NC(=O)CCCCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

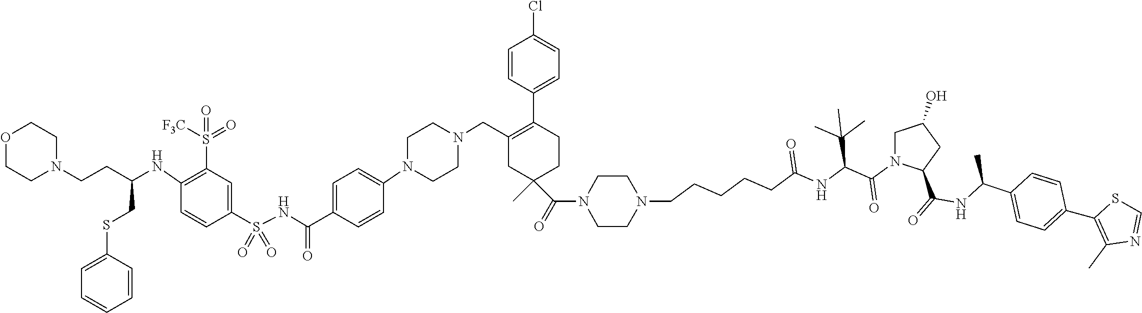 [SMILES: Cc1ncsc1-c1ccc([C@H](C)NC(=O)[C@@H]2C[C@@H](O)CN2C(=O)[C@@H](NC(=O)CCCCCN2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

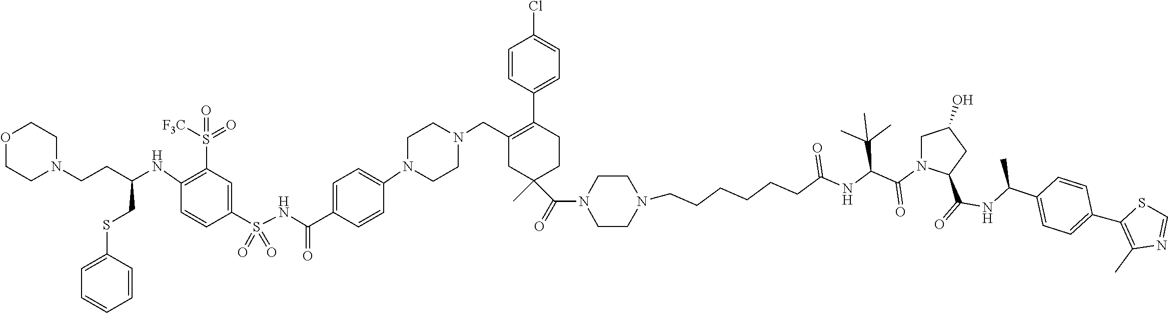 [SMILES: Cc1ncsc1-c1ccc([C@H](C)NC(=O)[C@@H]2C[C@@H](O)CN2C(=O)[C@@H](NC(=O)CCCCCCN2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

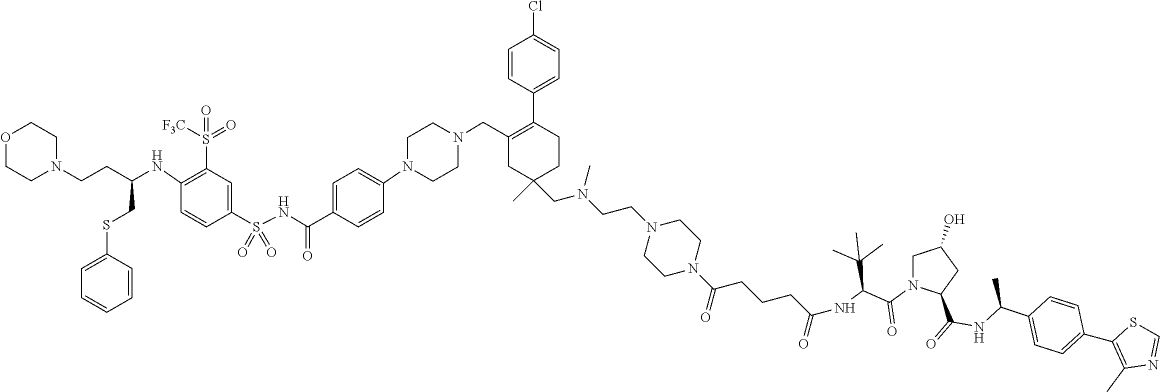 [SMILES: Cc1ncsc1-c1ccc([C@H](C)NC(=O)[C@@H]2C[C@@H](O)CN2C(=O)[C@@H](NC(=O)CCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

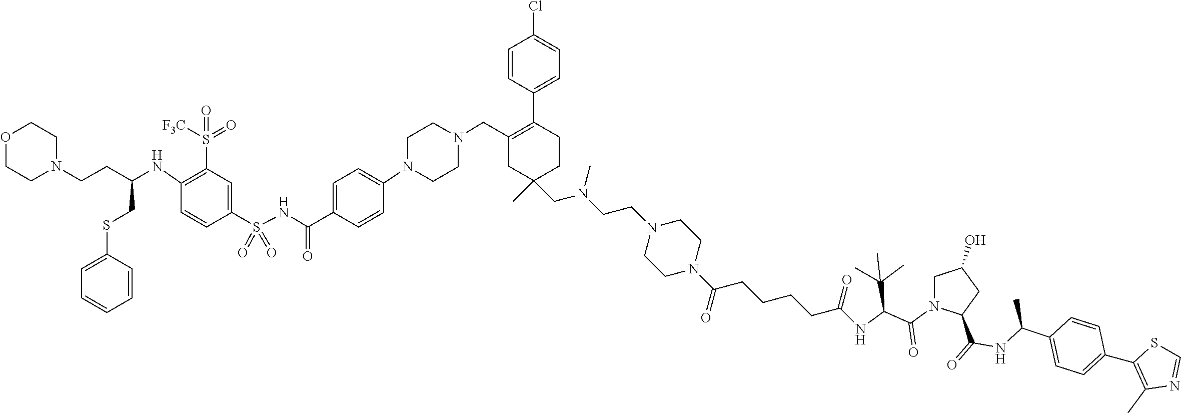 [SMILES: Cc1ncsc1-c1ccc([C@H](C)NC(=O)[C@@H]2C[C@@H](O)CN2C(=O)[C@@H](NC(=O)CCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

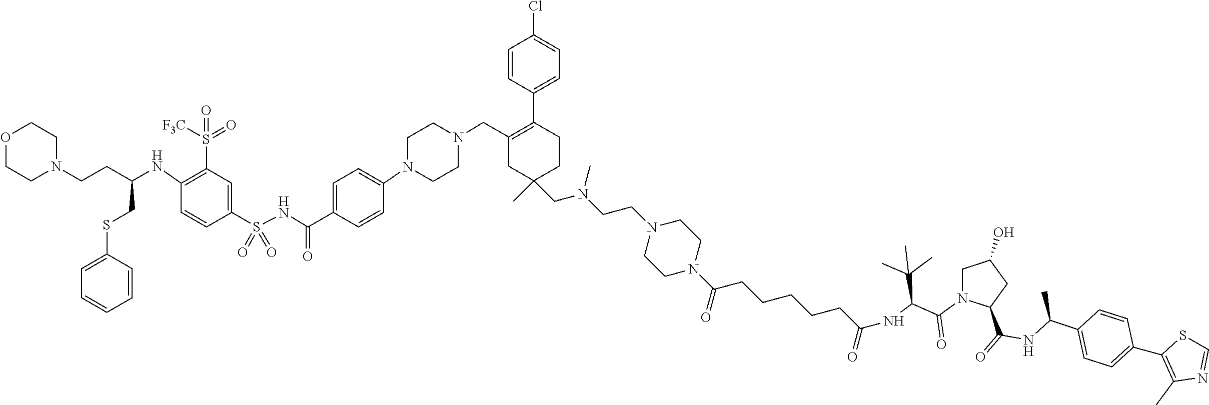 [SMILES: Cc1ncsc1-c1ccc([C@H](C)NC(=O)[C@@H]2C[C@@H](O)CN2C(=O)[C@@H](NC(=O)CCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

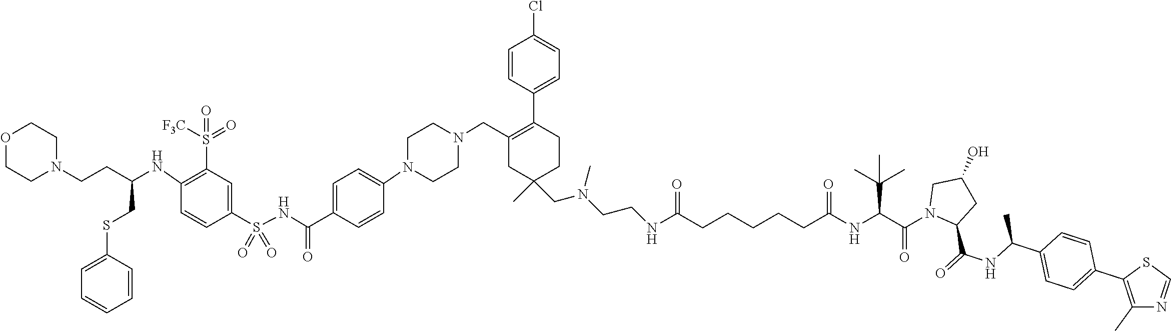 [SMILES: Cc1ncsc1-c1ccc([C@H](C)NC(=O)[C@@H]2C[C@@H](O)CN2C(=O)[C@@H](NC(=O)CCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

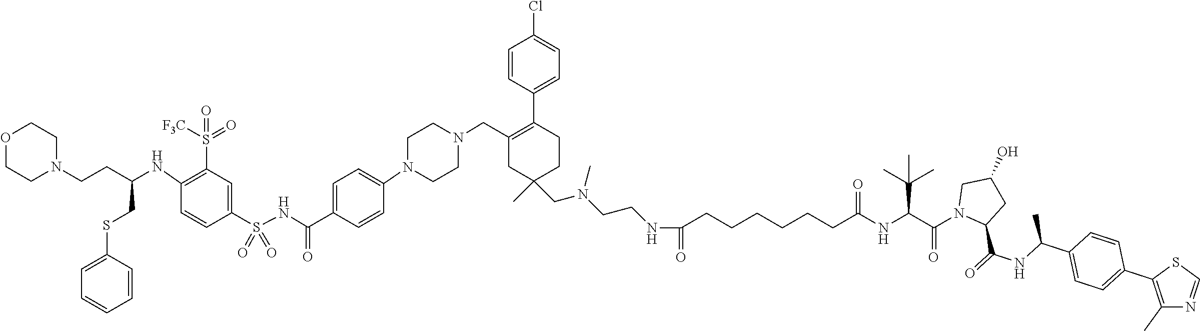 [SMILES: Cc1ncsc1-c1ccc([C@H](C)NC(=O)[C@@H]2C[C@@H](O)CN2C(=O)[C@@H](NC(=O)CCCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

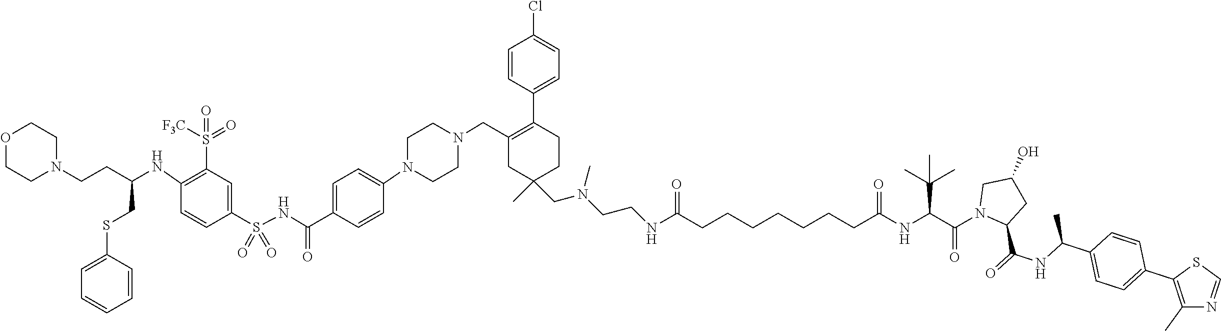 [SMILES: Cc1ncsc1-c1ccc([C@H](C)NC(=O)[C@@H]2C[C@@H](O)CN2C(=O)[C@@H](NC(=O)CCCCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

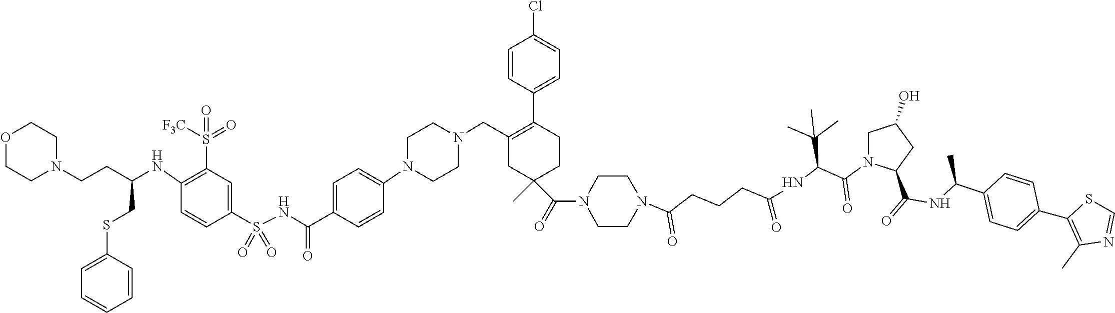 [SMILES: Cc1ncsc1-c1ccc([C@H](C)NC(=O)[C@@H]2C[C@@H](O)CN2C(=O)[C@@H](NC(=O)CCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

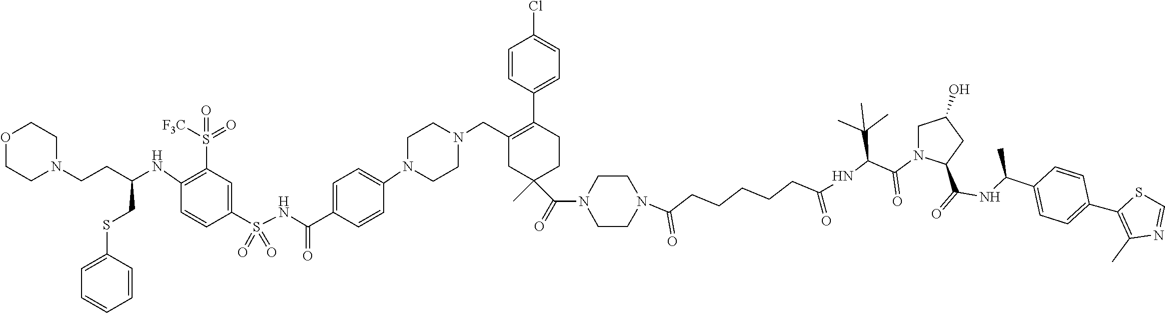 [SMILES: Cc1ncsc1-c1ccc([C@H](C)NC(=O)[C@@H]2C[C@@H](O)CN2C(=O)[C@@H](NC(=O)CCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

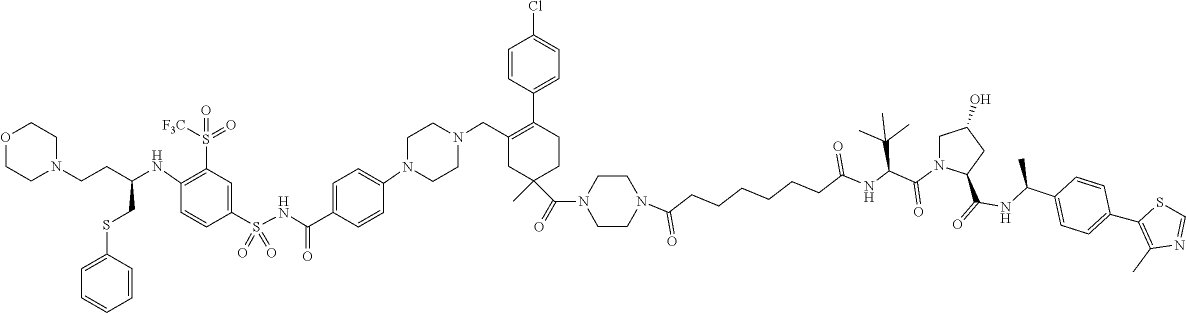 [SMILES: Cc1ncsc1-c1ccc([C@H](C)NC(=O)[C@@H]2C[C@@H](O)CN2C(=O)[C@@H](NC(=O)CCCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

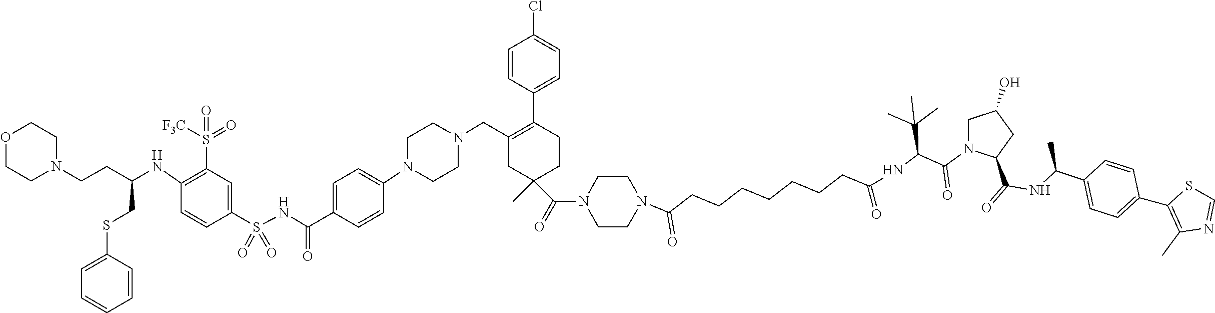 [SMILES: Cc1ncsc1-c1ccc([C@H](C)NC(=O)[C@@H]2C[C@@H](O)CN2C(=O)[C@@H](NC(=O)CCCCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

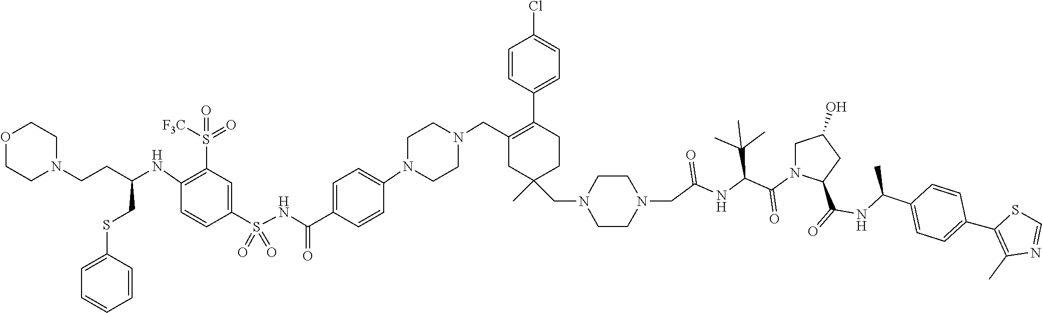 [SMILES: Cc1ncsc1-c1ccc([C@H](C)NC(=O)[C@@H]2C[C@@H](O)CN2C(=O)[C@@H](NC(=O)CN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

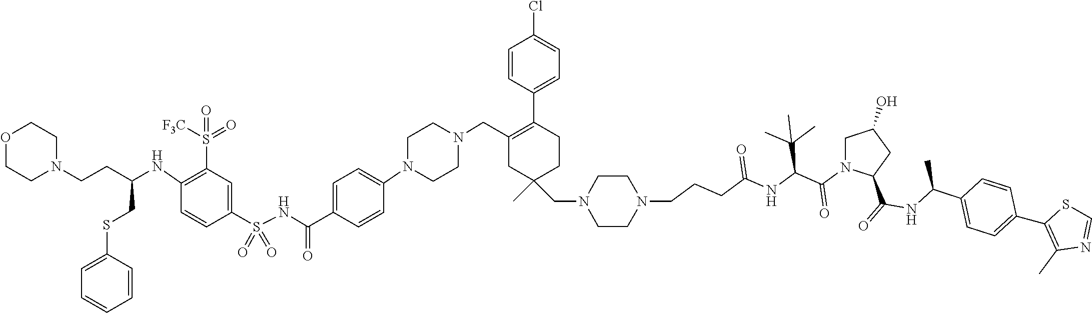 [SMILES: Cc1ncsc1-c1ccc([C@H](C)NC(=O)[C@@H]2C[C@@H](O)CN2C(=O)[C@@H](NC(=O)CCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

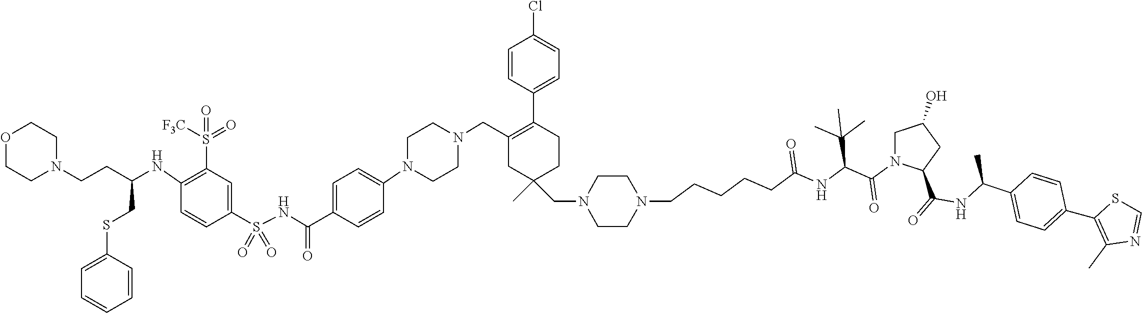 [SMILES: Cc1ncsc1-c1ccc([C@H](C)NC(=O)[C@@H]2C[C@@H](O)CN2C(=O)[C@@H](NC(=O)CCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

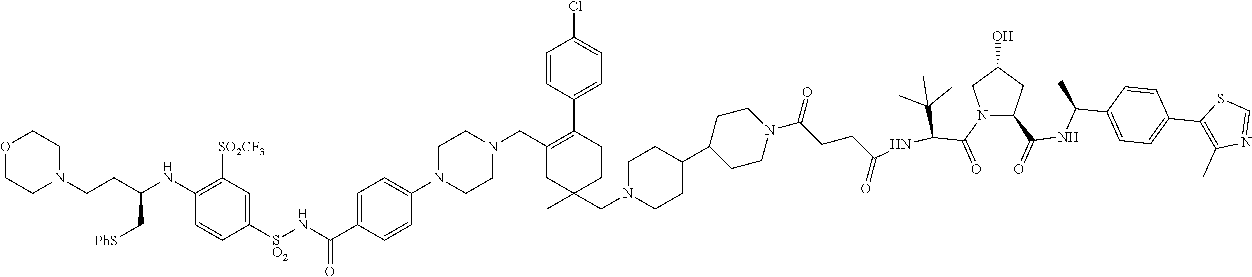

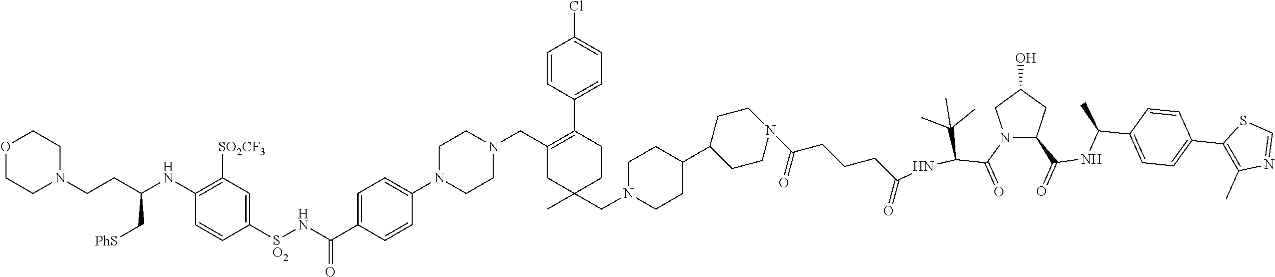

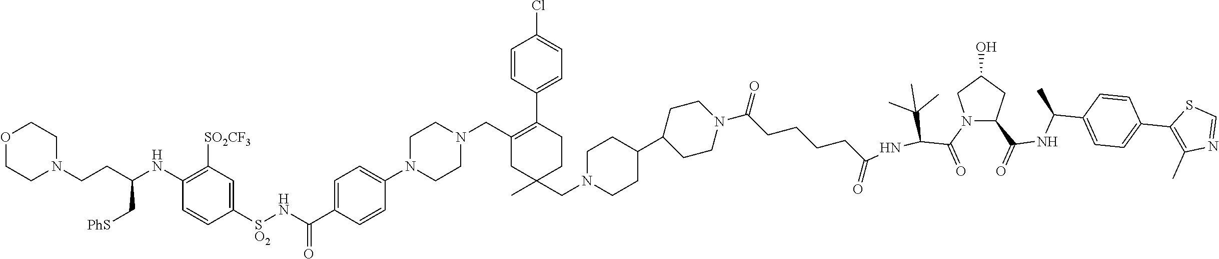

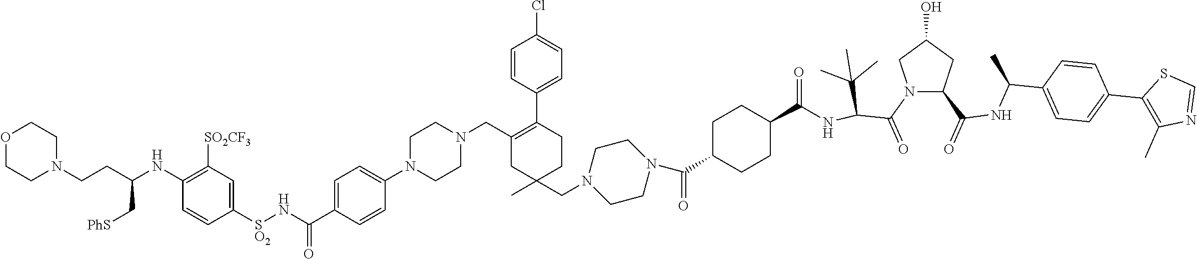

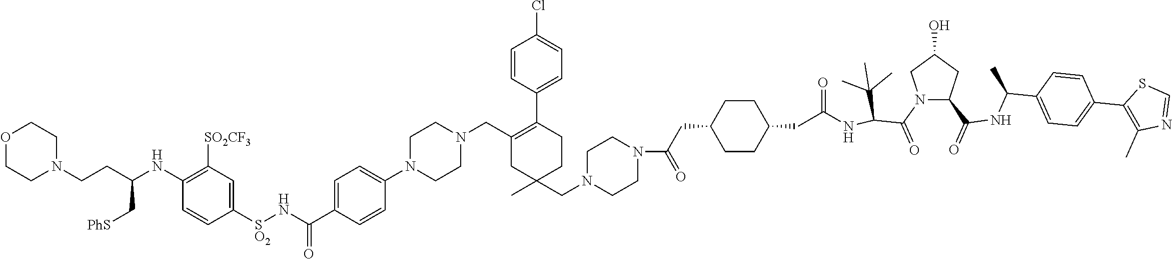

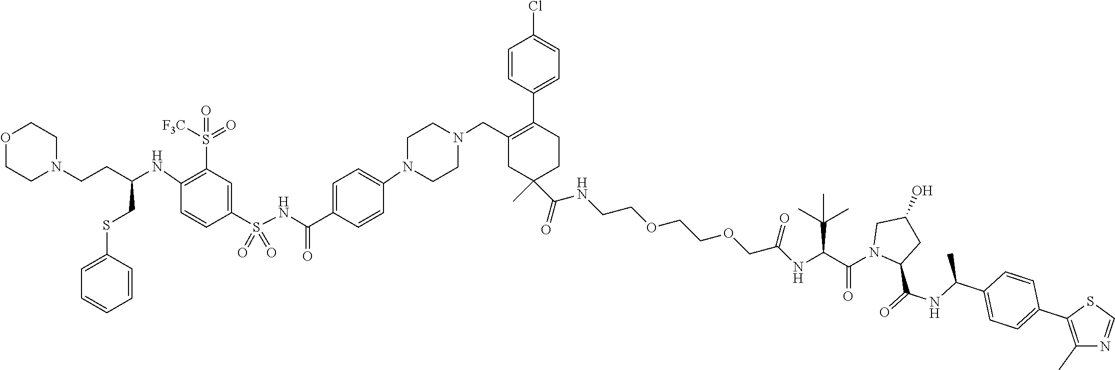 [SMILES: Cc1ncsc1-c1ccc([C@H](C)NC(=O)[C@@H]2C[C@@H](O)CN2C(=O)[C@@H](NC(=O)COCCOCCNC(=O)C2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

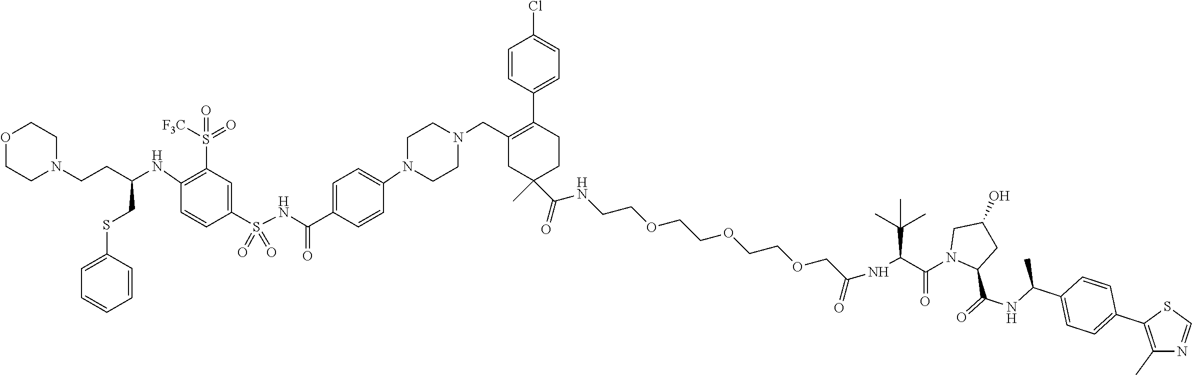 [SMILES: Cc1ncsc1-c1ccc([C@H](C)NC(=O)[C@@H]2C[C@@H](O)CN2C(=O)[C@@H](NC(=O)COCCOCCOCCNC(=O)C2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

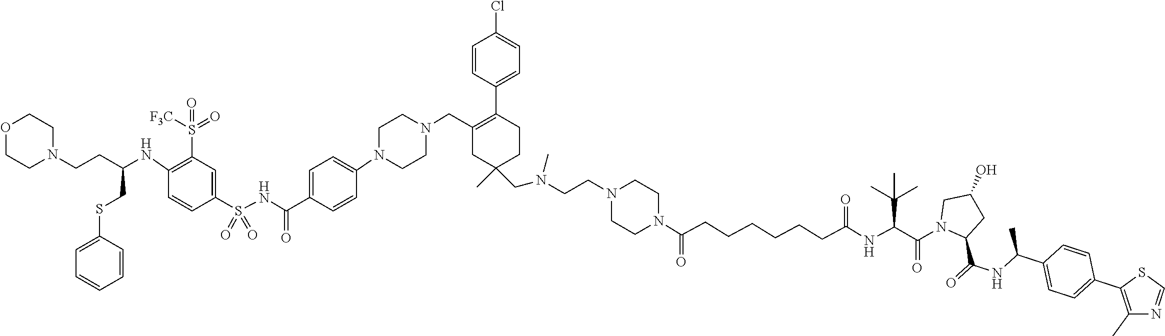 [SMILES: Cc1ncsc1-c1ccc([C@H](C)NC(=O)[C@@H]2C[C@@H](O)CN2C(=O)[C@@H](NC(=O)CCCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

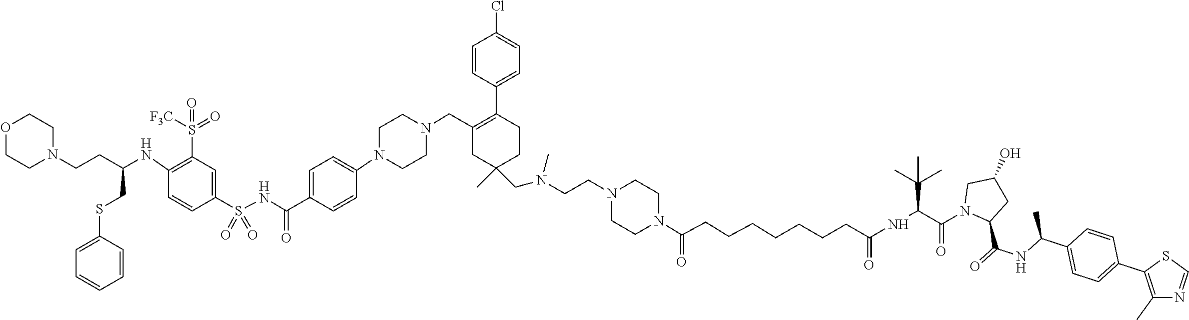 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

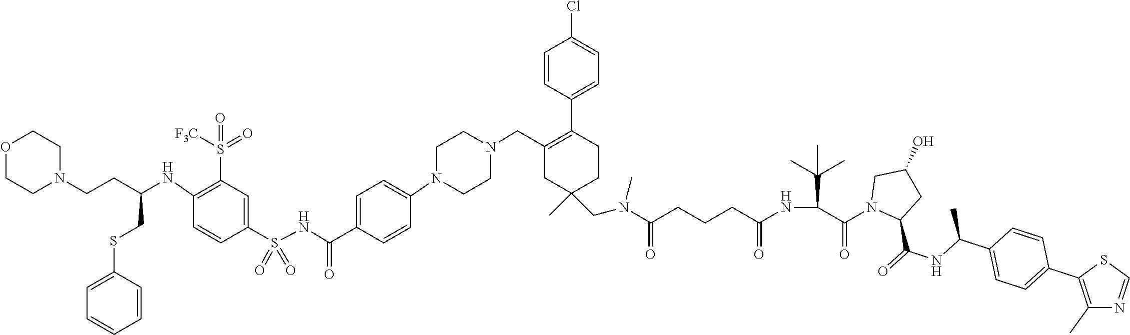 [SMILES: Cc1ncsc1-c1ccc([C@H](C)NC(=O)[C@@H]2C[C@@H](O)CN2C(=O)[C@@H](NC(=O)CCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

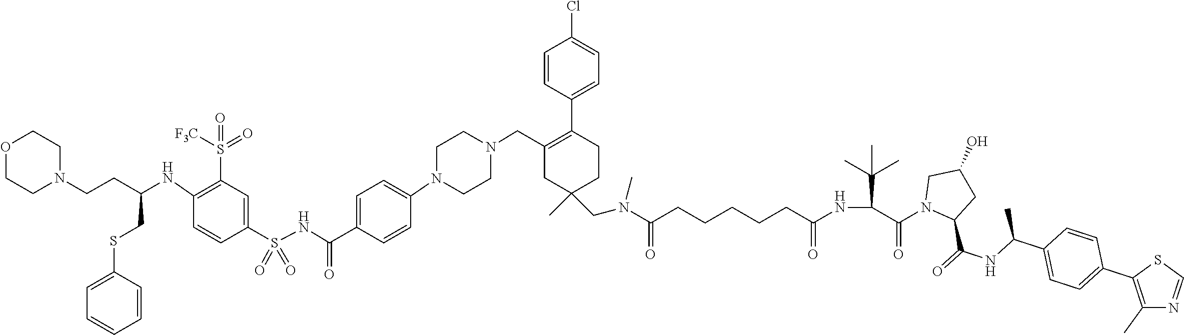 [SMILES: Cc1ncsc1-c1ccc([C@H](C)NC(=O)[C@@H]2C[C@@H](O)CN2C(=O)[C@@H](NC(=O)CCCCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

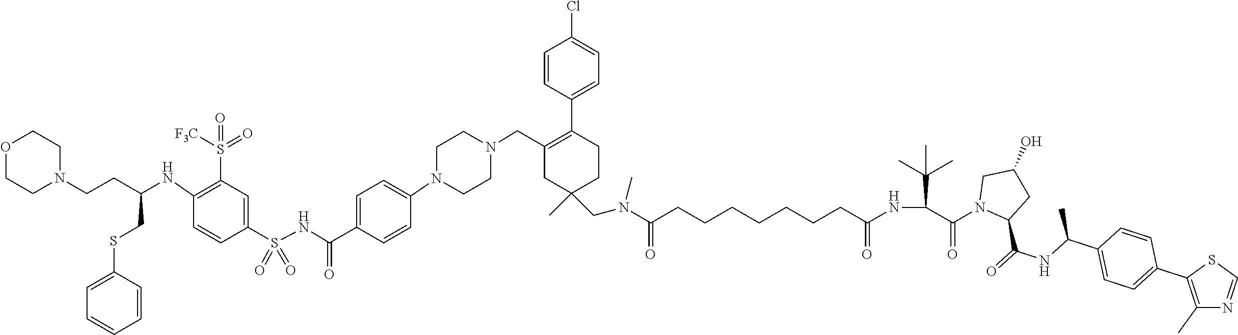 [SMILES: Cc1ncsc1-c1ccc([C@H](C)NC(=O)[C@@H]2C[C@@H](O)CN2C(=O)[C@@H](NC(=O)CCCCCCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

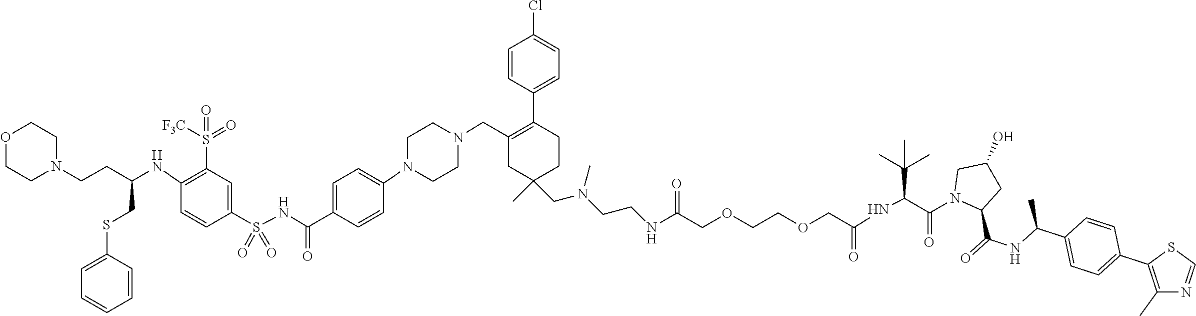 [SMILES: Cc1ncsc1-c1ccc([C@H](C)NC(=O)[C@@H]2C[C@@H](O)CN2C(=O)[C@@H](NC(=O)COCCOCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

In another aspect, the compound is a compound, or salt thereof, of any of the formulae herein (e.g., Formula (I) or Formula (II)), wherein the compound is not a compound provided in Table 1, or a salt, hydrate, solvate, or prodrug thereof. In another aspect, the compound is a compound, or salt thereof, of any of the formulae herein (e.g., Formula (I) or Formula (II)), wherein the compound is not a compound provided in Table 1, or a pharmaceutically acceptable salt thereof.

In another aspect, provided herein is a pharmaceutical composition comprising a compound provided herein (e.g., a compound of Formula (I) or Formula (II)), or a pharmaceutically acceptable salt, hydrate, solvate, or prodrug thereof, and a pharmaceutically acceptable carrier. In another aspect, the pharmaceutical composition further comprises an additional agent. In another aspect, the additional agent is an anti-cancer agent. In another aspect, the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

In another aspect, provided herein is a method of degrading Bcl-2 proteins, the method comprising administering an effective amount of a compound provided herein (e.g., a compound of Formula (I) or Formula (II)), or a pharmaceutically acceptable salt, hydrate, solvate, or prodrug thereof. In another aspect, the compound is administered in vitro. In another aspect, the compound is administered in vivo. In another aspect, the method further comprises administering the compound to a subject.

In another aspect, provided herein is a method of treating a disease or disorder in a subject in need thereof, the method comprising administering an effective amount of a compound provided herein (e.g., a compound of Formula (I) or Formula (II)), or a pharmaceutically acceptable salt, hydrate, solvate, or prodrug thereof. In another aspect, the disease is cancer. In another aspect, the cancer is a solid tumor. In another aspect, the cancer is small cell lung cancer. In another aspect, the cancer is chronic lymphocytic leukemia. In another aspect, the cancer is acute lymphoblastic leukemia. In another aspect, the subject is a mammal. In another aspect, the subject is a human.

In another aspect, provided herein is a method of treating a subject suffering from or susceptible to a disease or disorder, the method comprising administering an effective amount of a compound provided herein (e.g., a compound of Formula (I) or Formula (II)), or a pharmaceutically acceptable salt, hydrate, solvate, or prodrug thereof. In another aspect, the disease is cancer. In another aspect, the cancer is a solid tumor. In another aspect, the cancer is small cell lung cancer. In another aspect, the cancer is chronic lymphocytic leukemia. In another aspect, the cancer is acute lymphoblastic leukemia. In another aspect, the subject is a mammal. In another aspect, the subject is a human.

In another aspect, provided herein is a method of treating a Bcl-2-mediated cancer in a subject in need thereof, the method comprising administering an effective amount of a compound provided herein (e.g., a compound of Formula (I) or Formula (II)), or a pharmaceutically acceptable salt, hydrate, solvate, or prodrug thereof, wherein the platelet toxicity of the compound is less than the platelet toxicity of other Bcl-2 inhibitors. In another aspect, the Bcl-2-mediated cancer is chronic lymphocytic leukemia. In another aspect, the Bcl-2-mediated cancer is a solid tumor. In another aspect, the Bcl-2-mediated cancer is small cell lung cancer. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, provided herein is a method of treating a subject suffering from or susceptible to a Bcl-2-mediated cancer, the method comprising administering an effective amount of a compound provided herein (e.g., a compound of Formula (I) or Formula (II)), or a pharmaceutically acceptable salt, hydrate, solvate, or prodrug thereof, wherein the platelet toxicity of the compound is less than the platelet toxicity of other Bcl-2 inhibitors. In another aspect, the Bcl-2-mediated cancer is chronic lymphocytic leukemia. In another aspect, the Bcl-2-mediated cancer is solid tumor. In another aspect, the Bcl-2-mediated cancer is small cell lung cancer. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, provided herein is a method of treating a Bcl-2-mediated cancer in a subject in need thereof, the method comprising administering an effective amount of a compound provided herein (e.g., a compound of Formula (I) or Formula (II)), or a pharmaceutically acceptable salt, hydrate, solvate, or prodrug thereof, wherein the ratio of human platelet toxicity (IC₅₀) to anticancer activity (IC₅₀) of the compound is greater than one. In another aspect, the Bcl-2-mediated cancer is chronic lymphocytic leukemia. In another aspect, the Bcl-2-mediated cancer is solid tumor. In another aspect, the Bcl-2-mediated cancer is small cell lung cancer. In another aspect, the anticancer activity is measured in MOLT-4 cells. In another aspect, the anticancer activity is measured in RS4 cells. In some embodiments, the anticancer activity is higher in MOLT-4 cells than in RS4 cells. In certain embodiments, the anticancer activity is higher in RS4 cells than in MOLT-4 cells. In another aspect, the ratio is greater than 2.5, greater than 5, greater than 10, greater than 20, greater than 40, greater than 60, greater than 80, greater than 100, greater than 150, greater than 200, greater than 250, greater than 300, greater than 350, greater than 400, greater than 450, or greater than 500. In some embodiments, the ratio is greater than 2.5. In some embodiments, the ratio is greater than 5. In some embodiments, the ratio is greater than 10. In some embodiments, the ratio is greater than 20. In some embodiments, the ratio is greater than 40. In some embodiments, the ratio is greater than 60. In some embodiments, the ratio is greater than 80. In some embodiments, the ratio is greater than 100. In some embodiments, the ratio is greater than 150. In some embodiments, the ratio is greater than 200. In some embodiments, the ratio is greater than 250. In some embodiments, the ratio is greater than 300. In some embodiments, the ratio is greater than 350. In some embodiments, the ratio is greater than 400. In some embodiments, the ratio is greater than 450. In some embodiments, the ratio is greater than 500.

In another aspect, provided herein is a method of treating a subject suffering from or susceptible to a Bcl-2-mediated cancer, the method comprising administering an effective amount of a compound provided herein (e.g., a compound of Formula (I) or Formula (II)), or a pharmaceutically acceptable salt, hydrate, solvate, or prodrug thereof, such that ratio of human platelet toxicity (IC₅₀) to anticancer activity (IC₅₀) of the compound is greater than one. In another aspect, the Bcl-2-mediated cancer is chronic lymphocytic leukemia. In another aspect, the Bcl-2-mediated cancer is solid tumor. In another aspect, the Bcl-2-mediated cancer is small cell lung cancer. In another aspect, the anticancer activity is measured in MOLT-4 cells. In another aspect, the anticancer activity is measured in RS4 cells. In some embodiments, the anticancer activity is higher in MOLT-4 cells than in RS4 cells. In certain embodiments, the anticancer activity is higher in RS4 cells than in MOLT-4 cells. In another aspect, the ratio is greater than 2.5, greater than 5, greater than 10, greater than 20, greater than 40, greater than 60, greater than 80, greater than 100, greater than 150, greater than 200, greater than 250, greater than 300, greater than 350, greater than 400, greater than 450, or greater than 500. In some embodiments, the ratio is greater than 2.5. In some embodiments, the ratio is greater than 5. In some embodiments, the ratio is greater than 10. In some embodiments, the ratio is greater than 20. In some embodiments, the ratio is greater than 40. In some embodiments, the ratio is greater than 60. In some embodiments, the ratio is greater than 80. In some embodiments, the ratio is greater than 100. In some embodiments, the ratio is greater than 150. In some embodiments, the ratio is greater than 200. In some embodiments, the ratio is greater than 250. In some embodiments, the ratio is greater than 300. In some embodiments, the ratio is greater than 350. In some embodiments, the ratio is greater than 400. In some embodiments, the ratio is greater than 450. In some embodiments, the ratio is greater than 500.

Compounds of the present disclosure are bivalent compounds that promote the degradation of the anti-apoptotic Bcl-2 family of proteins. These bivalent compounds connect a Bcl-2 small molecule inhibitor or ligand to an E3 ligase binding moiety, such as von Hippel Landau (VHL) E3 ligase binding moiety (such as HIF-1α-derived (R)-hydroxyproline containing VHL E3 ligase ligands). VHL is part of the cullin-2 (CUL2) containing E3 ubiquitin ligase complex elongin BC-CUL2-VHL (known as CRL2VHL) responsible for degradation of the transcription factor HIF-1α. (R)-Hydroxyproline containing VHL E3 ligase ligands derived from HIF-1α have been identified with high affinity. The bivalent compounds can actively recruit anti-apoptotic Bcl-2 family of proteins to VHL E3 ligase, resulting in their degradation by ubiquitin proteasome system.

Platelets depend on Bcl-xL protein for survival. Thus, inhibition of Bcl-xL protein in platelets causes thrombocytopenia which limits the use of Bcl-xL inhibitors as cancer therapeutic agents. Given the well-documented importance of Bcl-xL in solid tumors and its contribution to drug resistance, strategies devised to minimize the on-target platelet toxicity associated with the inhibition of Bcl-xL could boost the therapeutic applications of drugs like ABT-263, a dual Bcl-2/Bcl-xL inhibitor, in cancer. The compounds of the present disclosure were designed to recruit VHL E3 ligase, which is minimally expressed in platelets for the targeted degradation of Bcl-xL.

Thus, the compounds provided herein (e.g., compounds of Formula (I) or Formula (II)) have reduced platelet toxicity compared with their corresponding Bcl-2/Bcl-xL inhibitors. Accordingly, the present disclosure provides compositions and methods for selectively degrading anti-apoptotic Bcl-2 family of proteins.

### BRIEF DESCRIPTION OF THE DRAWINGS

The present invention is further described below with reference to the following non-limiting examples and with reference to the following figures, in which:

FIGS. 1A to 1D show exemplary degradation of BCL-X_{L}, BCL-2, and MCL-1 by Western blotting analysis in Jurkat cells for 16 hours. The word “veh” refers to vehicle. FIG. 1A shows exemplary degradation by compound 26. FIG. 1B shows exemplary degradation by compound 32. FIG. 1C shows exemplary degradation by compound 65. FIG. 1D shows exemplary degradation by compound 68.

FIGS. 2A to 2C show that Compound 68 (#68) dose- and time-dependently degraded Bcl-xL and Bcl-2 in Jurkat cells, and the effects were long-lasting. FIG. 2A shows immunoblot analysis of Bcl-xL and Bcl-2 expression in Jurkat cells after treatment with increasing concentrations of the compound as indicated for 16 hours. FIG. 2B shows immunoblot analysis of Bcl-xL and Bcl-2 expression in Jurkat cells after treatment with the compound (100 nM) for various durations as indicated. FIG. 2C shows immunoblot analysis of Bcl-xL and Bcl-2 expression in Jurkat cells treated with the compound for 16 h followed by wash-off and then cultured without the compound for 0 to 48 as indicated.

FIGS. 3A to 3C show Compound 77 (#77) dose- and time-dependently degraded Bcl-xL and Bcl-2 in Jurkat cells, and the effects were long-lasting. FIG. 3A shows immunoblot analysis of Bcl-xL and Bcl-2 expression in Jurkat cells after treatment with increasing concentrations of the compound as indicated for 16 hours. FIG. 3B shows immunoblot analysis of Bcl-xL and Bcl-expression in Jurkat cells after treatment with the compound (100 nM) for various durations as indicated. FIG. 3C shows immunoblot analysis of Bcl-xL and Bcl-2 expression in Jurkat cells treated with the compound for 16 h followed by wash-off and then cultured without the compound for 0 to 48 as indicated.

FIG. 4 shows Compounds 68 and 77 do not affect Bcl-xL and Bcl-2 expression levels in human platelets after treatment the compounds for 16 h.

FIG. 5A shows Compound 77 and 753B formed ternary complexes with the VCB E3 ligase complex and Bcl-xL. FIG. 5B shows the structure of 753B (Lv et al., Nature Communications, 2021, 12, 6896).

### DETAILED DESCRIPTION

### Definitions

In order that the invention may be more readily understood, certain terms are first defined here for convenience.

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. As used herein, “treating” includes blocking, inhibiting, attenuating, modulating, reversing the effects of and reducing the occurrence of e.g., the harmful effects of a disorder.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

As used herein, “inhibiting” encompasses reducing and halting progression.

The term “modulate” refers to increases or decreases in the activity of a cell in response to exposure to a compound provided herein.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “protein” refers to series of amino acid residues connected one to the other by peptide bonds between the alpha-amino and carboxy groups of adjacent residues.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alter, add, or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The phrases “systemic administration,” “administered systemically,” “peripheral administration,” and “administered peripherally” as used herein mean the administration of a compound(s), drug or other material, such that it enters the patient's system and, thus, is subject to metabolism and other like processes.

The terms “composition” and “formulation” are used interchangeably.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) are outweighed by the therapeutically beneficial effects. An effective amount of a compound provided herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactically effective amount.

An effective amount of compound (i.e., an effective dosage) may range from about 0.005 μg/kg to about 200 mg/kg, preferably about 0.1 mg/kg to about 200 mg/kg, more preferably about 10 mg/kg to about 100 mg/kg of body weight. In other embodiments, the effective amount may range from about 1.0 pM to about 500 nM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with an effective amount of a compound can include a single treatment or, preferably, can include a series of treatments. In certain embodiments, an effective amount is the amount of a compound provided herein in a single dose. In certain embodiments, an effective amount is the combined amounts of a compound provided herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations). In one example, a subject is treated with a compound in the range of between about 0.005 μg/kg to about 200 mg/kg of body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The term “therapeutically effective amount” refers to that amount of the compound being administered sufficient to prevent development of or alleviate to some extent one or more of the symptoms of the condition or disorder being treated. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound provided herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “subject” refers to a human (i.e., male or female of any age group, e.g., pediatric subject (e.g., infant, child, or adolescent) or adult subject (e.g., young adult, middle-aged adult, or senior adult)) or non-human animal. In some embodiments, the term “subject” refers to animals such as mammals, including, but not limited to, primates (e.g., humans), cows, sheep, goats, horses, dogs, cats, rabbits, rats, mice and the like. In certain embodiments, the subject is a human. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition provided herein is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “chiral” refers to molecules which have the property of non-superimpo sability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Furthermore, the compounds provided herein include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds provided herein include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salts” or “pharmaceutically acceptable carrier” is meant to include salts of the active compounds which are prepared with relatively nontoxic acids or bases, depending on the particular substituents found on the compounds provided herein. When compounds provided herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds provided herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)).

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds provided herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions. The term “prodrug” includes compounds with moieties which can be metabolized in vivo.

Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

As used herein, the term “alkyl” refers to a straight-chained or branched hydrocarbon group containing 1 to 12 carbon atoms. The term “lower alkyl” refers to a C₁-C₆ alkyl chain. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Alkyl groups may be optionally substituted with one or more substituents. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁-s alkyl”). In some embodiments, a heteroalkyl group is a saturated group having 1 to carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to an unsaturated hydrocarbon chain that may be a straight chain or branched chain, containing 2 to 12 carbon atoms and at least one carbon-carbon double bond. In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Alkenyl groups may be optionally substituted with one or more substituents. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

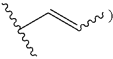

may be in the (E)- or (Z)-configuration.

The term “alkynyl” refers to an unsaturated hydrocarbon chain that may be a straight chain or branched chain, containing the 2 to 12 carbon atoms and at least one carbon-carbon triple bond (e.g., 1, 2, 3, or 4 triple bonds). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Alkynyl groups may be optionally substituted with one or more substituents. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁-alkynyl. In certain embodiments, the alkynyl group is a substituted C₁₋₂₀ alkynyl.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen”, “hal” or “halo” means —F, —Cl, —Br or —I. The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation and zero heteroatoms in the non-aromatic ring system. In some embodiments, a cycloalkyl group has 3 to 14 ring carbon atoms (“C₃₋₁₄ cycloalkyl”). In some embodiments, a cycloalkyl group has 3 to 13 ring carbon atoms (“C₃₋₁₃ cycloalkyl”). In some embodiments, a cycloalkyl group has 3 to 12 ring carbon atoms (“C₃₋₁₂ cycloalkyl”). In some embodiments, a cycloalkyl group has 3 to 11 ring carbon atoms (“C₃₋₁₁ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include cyclopropyl (C₃), cyclopropenyl (C₃), cyclobutyl (C₄), cyclobutenyl (C₄), cyclopentyl (C₅), cyclopentenyl (C₈), cyclohexyl (C₆), cyclohexenyl (C₆), cyclohexadienyl (C₆), and the like. Exemplary C₃₋₈ cycloalkyl groups include the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ cycloalkyl groups include the aforementioned C₃₋₁₀ cycloalkyl groups as well as cycloundecyl (C₁₁), spiro[5.5]undecanyl (C₁), cyclododecyl (C₁₂), cyclododecenyl (C₁₂), cyclotridecane (C₁₃), cyclotetradecane (C₁₄), and the like. As the foregoing examples illustrate, in certain embodiments, the cycloalkyl group is either monocyclic (“monocyclic cycloalkyl”) or polycyclic (e.g., containing a fused, bridged or spiro ring system such as a bicyclic system (“bicyclic cycloalkyl”) or tricyclic system (“tricyclic cycloalkyl”)) and can be saturated or can contain one or more carbon-carbon double or triple bonds. “Cycloalkyl” also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the carbocyclic ring system. Cycloalkyl groups may be optionally substituted with one or more substituents. Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

In some embodiments, “cycloalkyl” is a monocyclic, saturated cycloalkyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅10 cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the cycloalkyl includes 0, 1, or 2 C═C double bonds in the carbocyclic ring system, as valency permits.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the non-aromatic ring system is completely saturated. Heterocycloalkyl polycyclic ring systems can include one or more heteroatoms in one or both rings. “Heterocycloalkyl” also includes ring systems wherein the heterocycloalkyl ring, as defined above, is fused with one or more cycloalkyl groups wherein the point of attachment is either on the cycloalkyl or heterocycloalkyl ring, or ring systems wherein the heterocycloalkyl ring, as defined above, is fused with one or more aryl or heteroaryl groups, wherein the point of attachment is on the heterocycloalkyl ring, and in such instances, the number of ring members continue to designate the number of ring members in the heterocycloalkyl ring system. Heterocycloalkyl groups may be optionally substituted with one or more substituents. Unless otherwise specified, each instance of heterocycloalkyl is independently unsubstituted (an “unsubstituted heterocycloalkyl”) or substituted (a “substituted heterocycloalkyl”) with one or more substituents. In certain embodiments, the heterocycloalkyl group is an unsubstituted 3-14 membered heterocycloalkyl. In certain embodiments, the heterocycloalkyl group is a substituted 3-14 membered heterocycloalkyl. In certain embodiments, the heterocycloalkyl is substituted or unsubstituted, 3- to 7-membered, monocyclic heterocycloalkyl, wherein 1, 2, or 3 atoms in the heterocyclic ring system are independently oxygen, nitrogen, or sulfur, as valency permits. In one embodiment, 0, 1, 2, 3, or 4 atoms of each ring of a heterocycloalkyl group may be substituted by a substituent.

In some embodiments, a heterocycloalkyl group is a 5-10 membered non-aromatic ring system having ring carbon atoms and 1-4 ring heteroatoms, wherein each heteroatom is independently selected from nitrogen, oxygen, and sulfur (“5-10 membered heterocycloalkyl”). In some embodiments, a heterocycloalkyl group is a 5-8 membered non-aromatic ring system having ring carbon atoms and 1-4 ring heteroatoms, wherein each heteroatom is independently selected from nitrogen, oxygen, and sulfur (“5-8 membered heterocycloalkyl”). In some embodiments, a heterocycloalkyl group is a 5-6 membered non-aromatic ring system having ring carbon atoms and 1-4 ring heteroatoms, wherein each heteroatom is independently selected from nitrogen, oxygen, and sulfur (“5-6 membered heterocycloalkyl”). In some embodiments, the 5-6 membered heterocycloalkyl has 1-3 ring heteroatoms selected from nitrogen, oxygen, and sulfur. In some embodiments, the 5-6 membered heterocycloalkyl has 1-2 ring heteroatoms selected from nitrogen, oxygen, and sulfur. In some embodiments, the 5-6 membered heterocycloalkyl has 1 ring heteroatom selected from nitrogen, oxygen, and sulfur.

Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like. Exemplary 3-membered heterocycloalkyl groups containing 1 heteroatom include azirdinyl, oxiranyl, and thiiranyl. Exemplary 4-membered heterocycloalkyl groups containing 1 heteroatom include azetidinyl, oxetanyl, and thietanyl. Exemplary 5-membered heterocycloalkyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocycloalkyl groups containing 2 heteroatoms include dioxolanyl, oxathiolanyl and dithiolanyl. Exemplary 5-membered heterocycloalkyl groups containing 3 heteroatoms include triazolinyl, oxadiazolinyl, and thiadiazolinyl. Exemplary 6-membered heterocycloalkyl groups containing 1 heteroatom include piperidinyl, tetrahydropyranyl, dihydropyridinyl, and thianyl. Exemplary 6-membered heterocycloalkyl groups containing 2 heteroatoms include piperazinyl, morpholinyl, dithianyl, and dioxanyl. Exemplary 6-membered heterocycloalkyl groups containing 3 heteroatoms include triazinyl. Exemplary 7-membered heterocycloalkyl groups containing 1 heteroatom include azepanyl, oxepanyl and thiepanyl. Exemplary 8-membered heterocycloalkyl groups containing 1 heteroatom include azocanyl, oxecanyl and thiocanyl. Exemplary bicyclic heterocycloalkyl groups include indolinyl, isoindolinyl, dihydrobenzofuranyl, dihydrobenzothienyl, tetrahydro-benzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a hydrocarbon monocyclic, bicyclic or tricyclic aromatic ring system. In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more cycloalkyl or heterocycloalkyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

Heteroaryl groups may be optionally substituted with one or more substituents. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

The term “nucleophile” as used herein refers to an ion, atom, or functional group having a nucleophilic center, i.e., that is capable of seeking or reacting with an electrophilic center. In some embodiments, a nucleophile donates a pair of electrons to form a chemical bond (e.g., by reacting with an electrophile). In some embodiments, the nucleophile is neutral or negatively charged. Non-limiting examples of nucleophiles include uncharged compounds such as water, amines, mercaptans and alcohols, and charged moieties such as alkoxides, thiolates, carbanions, and various organic and inorganic anions. Non-limiting examples of anionic nucleophiles include simple anions such as hydroxides, azides, cyanides, thiocyanates, acetates, formates or chloroformates and bisulfites. Nucleophiles can also be provided as salts, such as, but not limited to, alkali metal salts (i.e., salts comprising an anionic nucleophile, such as an alkoxide, aryloxide, or thiolate, and an alkali metal cation, such as but not limited to a sodium (Na), potassium (K), lithium (Li), rubidium (Rb), or cesium (Cs) cation.

The term “electrophile” as used herein refers to an ion, atom, or functional group having an electrophilic center, i.e., that is attracted to an electron. In some embodiments, an electrophile participates in a chemical reaction by accepting a pair of electrons to form a chemical bond (e.g., by reacting with a nucleophile). In some embodiments, the electrophile is neutral or positively charged. Exemplary electrophilic groups are halide groups, such as bromide or chloride substituents, halogens (F, Cl, Br, or I); nitriles (CN); carboxylic esters (COO(LG)) where LG is a leaving group; carboxylic acids; carbonyls (CO); -aldehydes (—CHO), acetaldehydes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R—, —OC(═O)N(R^{bb})₂, —OC(═NR^{bb})R—, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. “Bcl-2” as used herein alone or as part of a group references to a member of the Bcl-2 family of proteins comprise the following Bcl-xL, MCL-1, Bcl-W, BFL-1/A1, Bcl-B, BAX, BAK, and BOK.

The term “proteolysis-targeting chimera” or “PROTAC” refers to a heterobifunctional molecule capable of inducing intracellular proteolysis. In some embodiments, a PROTAC comprises an E3-ubiquitin ligase binding molecule covalently linked to a component that binds the protein targeted for degradation.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Unless otherwise required by context, singular terms shall include pluralities, and plural terms shall include the singular. The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

When a range of values (“range”) is listed, it encompasses each value and sub-range within the range. A range is inclusive of the values at the two ends of the range unless otherwise provided. It will be understood that when a range is recited in the application, the ends of the range are specifically disclosed as if specifically recited. For example, a range of about 19% to about 99% specifically include a disclosure separately of 19% and separately of 99%.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Compounds

Compounds delineated herein (e.g., compounds of Formula (I) or Formula (I)) include salts, hydrates, solvates, and prodrugs thereof. They include all compounds delineated in schemes herein, whether intermediate or final compounds in a process.

Compounds provided herein can be obtained from natural sources or made or modified made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jahnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. For example, compounds of formulae herein can be made using methodology known in the art, including Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

The compounds provided herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds provided herein, even though only a single tautomeric form may be represented. All such isomeric forms of such compounds herein are expressly included in the present invention. All crystal forms and polymorphs of the compounds provided herein are expressly included in the present invention. All hydrate and solvate forms of the compounds provided herein are expressly included in the present invention. Also embodied are extracts and fractions comprising compounds provided herein. The term “isomers” is intended to include diastereoisomers, enantiomers, regioisomers, structural isomers, rotational isomers, tautomers, and the like. All such isomers of such compounds herein are expressly included in the present invention. For compounds which contain one or more stereogenic centers, e.g., chiral compounds, the methods provided herein may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound provided herein is administered to cells or a subject.

The compounds of the formulae herein can be synthesized using methodology similarly to that described in Chen, Q. Y.; Liu, Y.; Cai, W.; Luesch, H. Improved Total Synthesis and Biological Evaluation of Potent Apratoxin S4 Based Anticancer Agents with Differential Stability and Further Enhanced Activity. J. Med. Chem. 2014, 57 (7): p. 3011-302; and in WO2012/158933.

The present disclosure provides compounds which are in a salt form. In some embodiments, the salt is a pharmaceutically acceptable salt. Certain specific compounds provided herein contain both basic and acidic functionalities that allow the compounds to be converted into either base or acid addition salts. The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, provided herein are compounds which are in a prodrug form. Prodrugs of the compounds provided herein are those compounds that readily undergo chemical changes under physiological conditions to provide the compounds provided herein. Additionally, prodrugs can be converted to the compounds provided herein by chemical or biochemical methods in an ex vivo environment. For example, prodrugs can be slowly converted to the compounds provided herein when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are intended to be encompassed within the scope of the present invention. Certain compounds provided herein may exist in multiple crystalline or amorphous forms. In general, all physical forms are equivalent for the uses contemplated by the present invention and are intended to be within the scope of the present invention.

### Methods of Treatment

In another aspect, provided herein is a method of degrading Bcl-2 proteins, the method comprising administering an effective amount of a compound provided herein (e.g., a compound of Formula (I) or Formula (II)), or a pharmaceutically acceptable salt, hydrate, solvate, or prodrug thereof. In another aspect, the compound is administered in vitro. In another aspect, the compound is administered in vivo. In another aspect, the method further comprises administering the compound to a subject.

In another aspect, provided herein is a method of treating a disease or disorder in a subject in need thereof, the method comprising administering an effective amount of a compound provided herein (e.g., a compound of Formula (I) or Formula (II)), or a pharmaceutically acceptable salt, hydrate, solvate, or prodrug thereof. In another aspect, the disease is cancer. In another aspect, the cancer is a solid tumor. In another aspect, the cancer is chronic lymphocytic leukemia. In another aspect, the subject is a mammal. In another aspect, the subject is a human.

In another aspect, provided herein is a method of treating a subject suffering from or susceptible to a disease or disorder, the method comprising administering an effective amount of a compound provided herein (e.g., a compound of Formula (I) or Formula (II)), or a pharmaceutically acceptable salt, hydrate, solvate, or prodrug thereof. In another aspect, the disease is cancer. In another aspect, the cancer is a solid tumor. In another aspect, the cancer is chronic lymphocytic leukemia. In another aspect, the subject is a mammal. In another aspect, the subject is a human.

In another aspect, provided herein is a method of treating a Bcl-2-mediated cancer in a subject in need thereof, the method comprising administering an effective amount of a compound provided herein (e.g., a compound of Formula (I) or Formula (II)), or a pharmaceutically acceptable salt, hydrate, solvate, or prodrug thereof, such that platelet toxicity is reduced relative to other Bcl-2 inhibitors. In another aspect, the Bcl-2-mediated cancer is chronic lymphocytic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C₁), TW-37, BM-1252 (APG-1252), or A-1155463. In another aspect, the other Bcl-2 inhibitor is venetoclax or ABT-263.

In another aspect, provided herein is a method of treating a subject suffering from or susceptible to a Bcl-2-mediated cancer, the method comprising administering an effective amount of a compound provided herein (e.g., a compound of Formula (I) or Formula (II)), or a pharmaceutically acceptable salt, hydrate, solvate, or prodrug thereof, such that platelet toxicity is reduced relative to other Bcl-2 inhibitors. In another aspect, the Bcl-2-mediated cancer is chronic lymphocytic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97Cl), TW-37, BM-1252 (APG-1252), orA-1155463. In another aspect, the other Bcl-2 inhibitor is venetoclax or ABT-263.

In another aspect, provided herein is a method of treating a Bcl-2-mediated cancer in a subject in need thereof, the method comprising administering an effective amount of a compound provided herein (e.g., a compound of Formula (I) or Formula (II)), or a pharmaceutically acceptable salt, hydrate, solvate, or prodrug thereof, such that ratio of human platelet toxicity (IC₅₀) to anticancer activity (IC₅₀) is less than that of other Bcl-2 inhibitors. In another aspect, the Bcl-2-mediated cancer is chronic lymphocytic leukemia. In another aspect, the other Bcl-2 inhibitor is venetoclax or ABT-263. In another aspect, the anticancer activity is measured in MOLT-4 cells. In another aspect, the ratio is greater than 1. In another aspect, the ratio is greater than 10. In another aspect, the ratio is greater than 20. In another aspect, the ratio is greater than 40.

In another aspect, provided herein is a method of treating a subject suffering from or susceptible to a Bcl-2-mediated cancer, the method comprising administering an effective amount of a compound provided herein (e.g., a compound of Formula (I) or Formula (II)), or a pharmaceutically acceptable salt, hydrate, solvate, or prodrug thereof, such that ratio of human platelet toxicity (IC₅₀) to anticancer activity (IC₅₀) is less than that of other Bcl-2 inhibitors. In another aspect, the Bcl-2-mediated cancer is chronic lymphocytic leukemia. In another aspect, the other Bcl-2 inhibitor is venetoclax or ABT-263. In another aspect, the anticancer activity is measured in MOLT-4 cells. In another aspect, the ratio is greater than 1. In another aspect, the ratio is greater than 10. In another aspect, the ratio is greater than 20. In another aspect, the ratio is greater than 40.

The present disclosure encompasses a method of selectively killing one or more cancer cells in a sample, the method comprising contacting a composition comprising an effective amount of a compound of Formula (I) with the sample. In another aspect, the present disclosure encompasses a method of selectively killing one or more cancer cells in a subject in need thereof, the method comprising administering to the subject a composition comprising a therapeutically effective amount of a compound of Formula (I).

By “selectively killing one or more cancer cells,” it is meant that a composition provided herein does not appreciably kill non-cancer cells at the same concentration. In one embodiment, a composition provided herein has reduced platelet toxicity and retained or improved toxicity in cancer cells when compared to other BCL-2 inhibitors. Accordingly, the median lethal dose or LD50 of the inhibitor in non-cancer cells may be about 5 to about 50 times higher than the LD50 of the inhibitor in cancer cells. As used herein, the LD50 is the concentration of inhibitor required to kill half the cells in the cell sample. For example, the LD50 of the inhibitor in non-cancer cells may be greater than about 5, about 6, about 7, about 8, about 9 or about 10 times higher than the LD50 of the inhibitor in cancer cells. Alternatively, the LD50 of the inhibitor in non-cancer cells may be greater than about 10, about 15, about 20, about 25, about 30, about 35, about 40, about 45, or about 50 times higher than the LD50 of the inhibitor in cancer cells. Additionally, the LD50 of the inhibitor in non-cancer cells may be greater than 50 times higher than the LD50 of the inhibitor in cancer cells. In a specific embodiment, the LD50 of the inhibitor in non-cancer cells is greater than 10 times higher than the LD500 of the inhibitor in cancer cells. In another specific embodiment, the LD50 of the inhibitor in non-cancer cells is greater than 20 times higher than the LD50 of the inhibitor in cancer cells.

Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas, breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, choriocarcinoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, glioblastoma, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In certain embodiments, a cancer is selected from the group consisting of synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).

### Pharmaceutical Compositions

In one aspect, provided herein is a pharmaceutical composition comprising a compound of any of the formulae herein (e.g., a compound of Formula (I) or Formula (II)), or a pharmaceutically acceptable salt hydrate, solvate, or prodrug thereof, and a pharmaceutically acceptable carrier.

In another embodiment, provided herein is a pharmaceutical composition wherein the composition comprises a compound of Formula (I), or a pharmaceutically acceptable salt hydrate, solvate, or prodrug thereof, and a pharmaceutically acceptable carrier. In another embodiment, provided herein is a pharmaceutical composition wherein the composition comprises a compound of Formula (II), or a pharmaceutically acceptable salt hydrate, solvate, or prodrug thereof, and a pharmaceutically acceptable carrier. In another aspect, the composition further comprises an additional agent. In another aspect, the additional agent is an anti-cancer agent. In another aspect, the anti-cancer agent is alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors include, but are not limited to, amsacrine, etoposide (VP-16), irinotecan, mitoxantrone, RFS 2000, teniposide, and topotecan.

Non-limiting examples of suitable anti-hormonal agents such as aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib; angiogeneisis inhibitors such as angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide; and growth inhibitory polypeptides such as bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1BB ligand, andApo-1 ligand.

Non-limiting examples of photodynamic therapeutic agents include aminolevulinic acid, methyl aminolevulinate, retinoids (alitretinon, tamibarotene, tretinoin), and temoporfin.

Other antineoplastic agents include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

In one aspect, provided herein is a kit comprising an effective amount of a compound of any of the formulae herein (e.g., a compound of Formula (I) or Formula (II)), or a pharmaceutically acceptable salt, hydrate, solvate, or prodrug thereof, in unit dosage form, together with instructions for administering the compound to a subject suffering from or susceptible to cancer. In another aspect, the cancer is a solid tumor. In another aspect, the cancer is chronic lymphocytic leukemia.

In some embodiments, a compound provided herein, or a pharmaceutically acceptable salt, hydrate, solvate, or prodrug thereof, is administered to the subject using a pharmaceutically acceptable formulation, e.g., a pharmaceutically acceptable formulation that provides sustained delivery of the compound to a subject for at least 12 hours, 24 hours, 36 hours, 48 hours, one week, two weeks, three weeks, or four weeks after the pharmaceutically acceptable formulation is administered to the subject.

Actual dosage levels and time course of administration of the active ingredients in the pharmaceutical compositions provided herein may be varied so as to obtain an amount of the active ingredient which is effective to achieve the desired therapeutic response for a particular patient, composition, and mode of administration, without being toxic (or unacceptably toxic) to the patient.

In use, at least one compound provided herein, or a pharmaceutically acceptable salt, hydrate, solvate, or prodrug thereof, is administered in a pharmaceutically effective amount to a subject in need thereof in a pharmaceutical carrier by intravenous, intramuscular, subcutaneous, or intracerebro ventricular injection or by oral administration or topical application. A compound provided herein may be administered alone or in conjunction with a second, different therapeutic. By “in conjunction with” is meant together, substantially simultaneously or sequentially. In one embodiment, a compound provided herein is administered acutely. The compound provided herein may therefore be administered for a short course of treatment, such as for about 1 day to about 1 week. In another embodiment, the compound provided herein may be administered over a longer period of time to ameliorate chronic disorders, such as, for example, for about one week to several months depending upon the condition to be treated.

As used herein, “pharmaceutically effective amount” refers to an amount of a compound provided herein, high enough to significantly positively modify the condition to be treated but low enough to avoid serious side effects (at a reasonable benefit/risk ratio), within the scope of sound medical judgment. A pharmaceutically effective amount of a compound provided herein will vary with the particular goal to be achieved, the age and physical condition of the patient being treated, the severity of the underlying disease, the duration of treatment, the nature of concurrent therapy and the specific apratoxin compound employed. For example, a therapeutically effective amount of a compound provided herein administered to a child or a neonate will be reduced proportionately in accordance with sound medical judgment. The effective amount of a compound provided herein will thus be the minimum amount which will provide the desired effect.

The compound or composition thereof may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound provided herein in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations provided herein are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

### EXAMPLES

The present invention will now be demonstrated using specific examples that are not to be construed as limiting.

### Compound Preparation

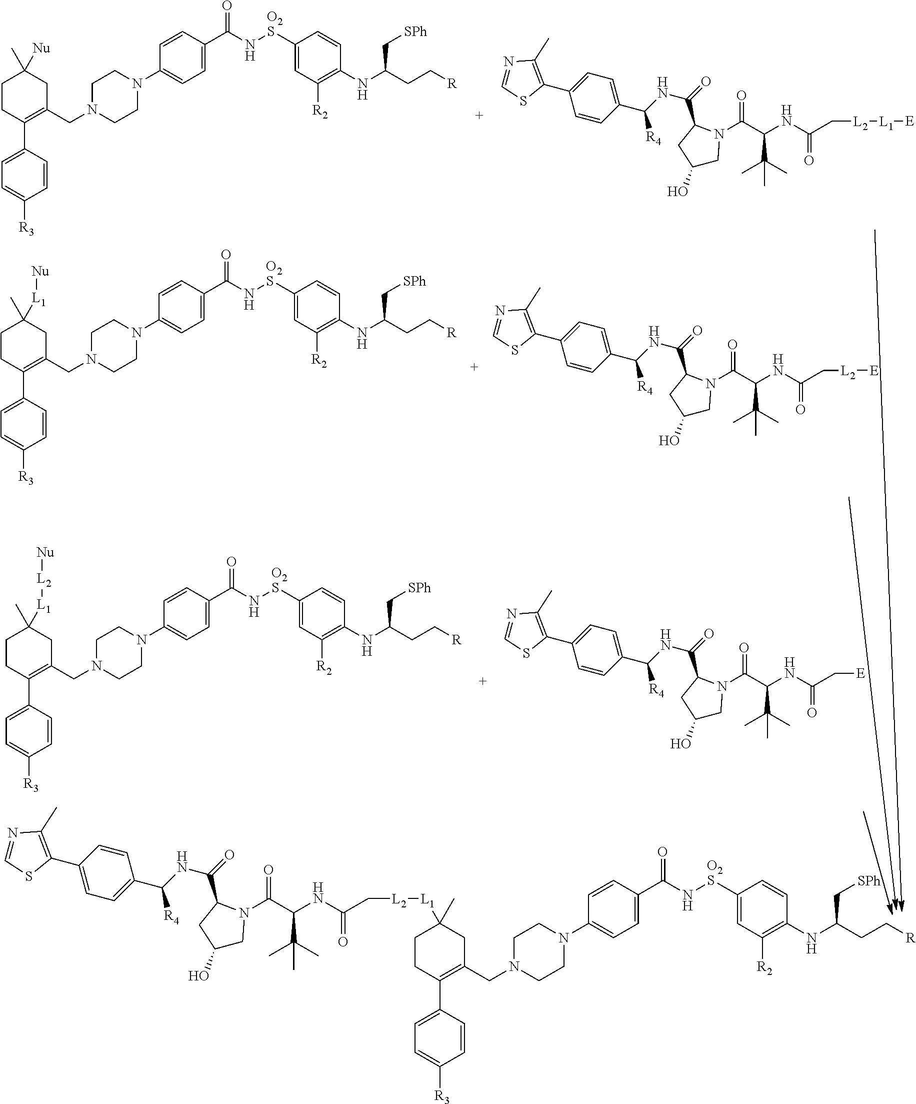

Compounds provided herein can be made or modified by a variety of means known in the art of organic synthesis. For example, among various organic synthesis methodologies known in the art, compounds provided herein can be synthesized according to the reaction scheme above. E represents an electrophile and Nu represents a nucleophile. R is R₁ or morpholine. R₁, R₂, R₃, R₄, L₁, and L₂ are as defined herein. In some embodiments, the nucleophile is an amine and the electrophile is a carboxylic acid.

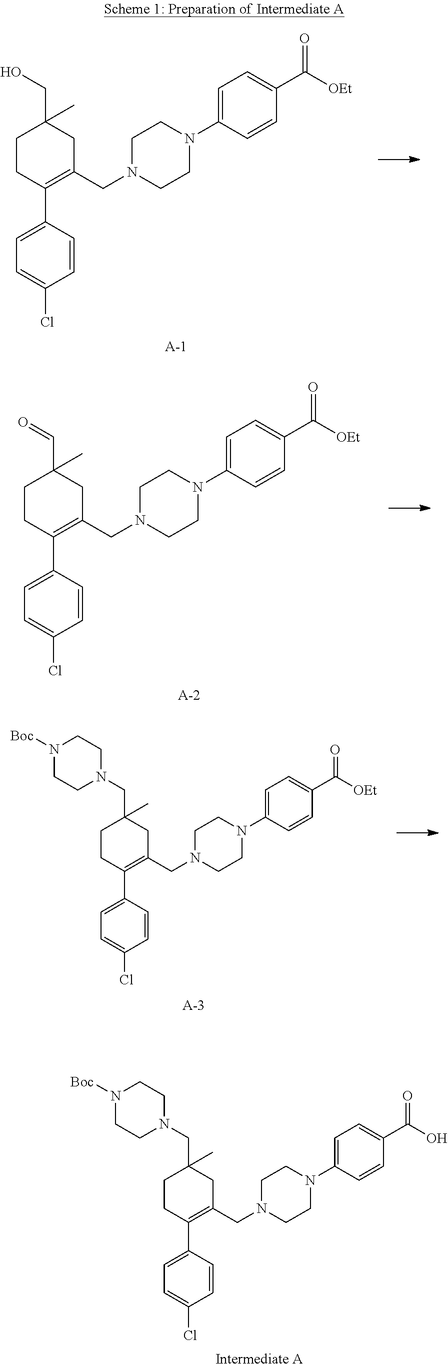

Preparation of ethyl 4-(4-((4′-chloro-4-formyl-4-methyl-3,4,5,6-tetrahydro-[1,1′-biphenyl]-2-yl)methyl)piperazin-1-yl)benzoate (A-2): Under an argon atmosphere, to a solution of DMSO (440 μL, 6.21 mmol) in DCM (10 mL) was added oxalyl chloride (351 μL, 4.14 mmol) dropwise at −78° C. with stirring. The resulting mixture was stirred at −78° C. for an additional 15 min. Then a solution of A-1 (1 g, 2.07 mmol) in DCM/DMSO (20 mL/5 mL) was added dropwise. The mixture was stirred at −78° C. for an extra 30 min after completing the addition. TEA (1.73 mL, 12.42 mmol) was added to the mixture at −78° C., then the dry-ice/acetone bath was removed. The reaction mixture was warmed up to room temperature and stirred for an additional 30 min. The reaction mixture was added to water and extracted with DCM. The combined organic layers were washed with 10% aq. Na₂S₂O₃ and sat. aq. NH₄Cl, dried over Na₂SO₄, filtered, and concentrated. The residue was purified by flash column chromatography (0% to 40% of EtOAc in hexanes) to afford A-2 (760 mg, 76% yield). ¹H NMR (600 MHz, CDCl₃) δ 9.56 (s, 1H), 7.94-7.91 (m, 2H), 7.31-7.29 (m, 2H), 7.00-6.96 (m, 2H), 6.85-6.82 (in, 2H), 4.34 (q, J=7.1 Hz, 2H), 3.30 (t, J=5.1 Hz, 4H), 2.86 (q, J=12.6 Hz, 2H), 2.69 (dd, J=18.1, 1.9 Hz, 1H), 2.47-2.40 (m, 2H), 2.40-2.25 (m, 4H), 2.11-2.04 (m, 1H), 2.04-1.97 (in, 1H), 1.68-0.58 (m, 1H), 1.38 (t, J=7.1 Hz, 3H), 1.18 (s, 3H) ppm. LC/MS (ESI) m/z 481.1; [M+H]⁺ calcd for C₂₈H₃₄ClN₂O₃⁺: 481.23.

Preparation of tert-butyl 4-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)piperazine-1-carboxylate (A-3): NaBH(OAc)₃ (771 mg, 3.64 mmol) was added in portions to a solution of A-2 (350 mg, 0.727 mmol), 1-Boc-piperazine (162 mg, 0.87 mmol), and TEA (607 μL, 4.37 mmol) in DCM (5 mL). The resulting mixture was stirred at room temperature overnight. The reaction mixture was washed with sat. aq. NH₄Cl, dried over Na₂SO₄, filtered, and concentrated. The residue was purified by flash column chromatography (0% to 40% of EtOAc in hexanes) to afford compound A-3 (400 mg, 84% yield). ¹H NMR (600 MHz, CDCl₃) δ 7.94-7.90 (m, 2H), 7.32-7.27 (m, 2H), 7.03-7.00 (m, 2H), 6.86-6.81 (m, 2H), 4.34 (q, J=7.1 Hz, 2H), 3.42 (t, J 4=5.0 Hz, 4H), 3.27 (t, J=5.1 Hz, 4H), 2.82 (s, 2H), 2.59-2.11 (m, 14H), 1.95 (d, 1H), 1.67-1.57 (m, 1H), 1.48 (s, 9H), 1.38 (t, J=7.1 Hz, 3H), 0.97 (s, 3H) ppm. LC/MS (ESI) m/z 651.2; [M+H]⁺ calcd for C₃₇H₅₂ClN₄O₄⁺: 651.37.

Preparation of 4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic acid (Intermediate A): To a solution of A-3 (400 mg, 0.61 mmol) in MeOH/THF (4 mL/4 mL) was added a solution of LiOH·H₂O (129 mg, 3.07 mmol) in water (1 mL), then stirred at 45° C. overnight. The reaction mixture was concentrated to remove the organic solvents, then adjusted to pH=5-6 with 10% aq. citric acid and extracted with EtOAc. The combined organic layers were washed with water and sat. aq. brine, dried over Na₂SO₄, filtered, and concentrated. The residue was purified by flash column chromatography (0% to 70% of EtOAc in hexanes) to afford intermediate A (340 mg, 89% yield). ¹H NMR (600 MHz, CDCl₃) δ 7.98-7.92 (m, 2H), 7.33-7.27 (m, 2H), 7.03-7.00 (m, 2H), 6.86-6.81 (m, 2H), 3.41 (t, J=4.9 Hz, 4H), 3.30 (t, J=5.2 Hz, 4H), 2.85 (s, 2H), 2.59-2.13 (m, 14H), 1.97 (d, 1H), 1.67-1.58 (m, 1H), 1.47 (s, 9H), 0.97 (s, 3H) ppm. LC/MS (ESI) m/z 623.2; [M+H]⁺ calcd for C₃₅H₄₈ClN₄O₄⁺: 623.34.

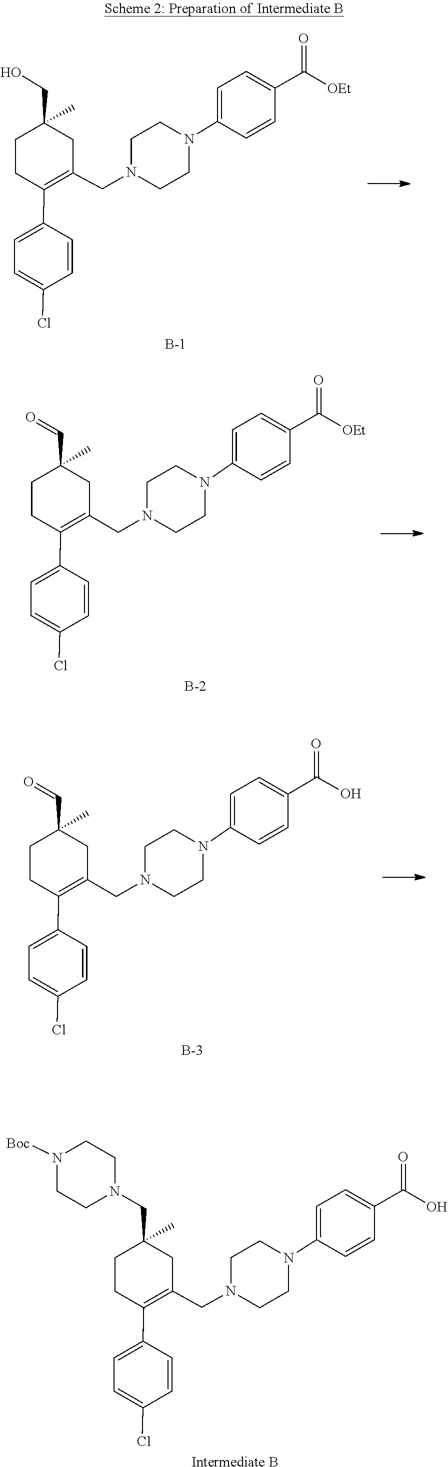

Preparation of ethyl (R)-4-(4-((4′-chloro-4-formyl-4-methyl-3,4,5,6-tetrahydro-[1,1′-biphenyl]-2-yl)methyl)piperazin-1-yl)benzoate (B-2): The preparation was preformed according to the first step in the preparation of Intermediate A with B-1 (3 g, 6.21 mmol) as a starting material. B-2 (2.3 g) was obtained in 77% yield. ¹H NMR (600 MHz, Chloroform-d) δ 9.56 (s, 1H), 7.95-7.89 (m, 2H), 7.33-7.27 (m, 2H), 7.01-6.95 (m, 2H), 6.87-6.81 (m, 2H), 4.34 (q, J=7.1 Hz, 2H), 3.30 (t, J=5.1 Hz, 4H), 2.86 (q, J=12.7 Hz, 2H), 2.73-2.66 (m, 1H), 2.48-2.41 (m, 2H), 2.40-2.26 (m, 4H), 2.12-2.04 (m, 1H), 2.05-1.99 (m, 1H), 1.69-1.60 (m, 1H), 1.38 (t, J=7.1 Hz, 3H), 1.18 (s, 3H) ppm. LC/MS (ESI) m/z 481.2; [M+H]⁺ calcd for C₂₈H₃₄ClN₂O₃⁺: 481.23.

Preparation of (R)-4-(4-((4′-chloro-4-formyl-4-methyl-3,4,5,6-tetrahydro-[1,1′-biphenyl]-2-yl)methyl)piperazin-1-yl)benzoic acid (B-3): To a solution of B-2 (2.3 g, 4.78 mmol) in MeOH/THF (25 mL/25 mL) was added a solution of LiOH·H₂O (602 mg, 14.35 mmol) in water (6 mL), then the resulting mixture was stirred at 40° C. overnight. The reaction mixture was concentrated to remove the organic solvents, then adjusted to pH=5-6 with 10% aq. citric acid. The precipitate was collected via filtration, then washed with water and dried under vacuum to afford B-3 (2.0 g, 92% yield). ¹H NMR (600 MHz, Methanol-d₄) δ 9.56 (s, 1H), 7.91 (d, J=9.0 16 Hz, 2H), 7.40-7.37 (m, 2H), 7.11-7.08 (m, 2H), 7.00-6.95 (m, 2H), 3.53-3.42 (m, 4H), 3.04-2.89 (m, 3H), 2.83 (s, 2H), 2.68 (dd, J=17.5, 1.9 Hz, 1H), 2.44-2.32 (m, 3H), 2.17-2.06 (m, 2H), 1.78-1.68 (m, 1H), 1.21 (s, 3H) ppm. LC/MS (ESI) m/z 453.1; [M+H]⁺ calcd for C₂₆H₂₉ClN₂O₃⁺: 452.98.

Preparation of (R)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic acid (Intermediate B): NaBH(OAc)₃ (468 mg, 2.21 mmol) was added to a solution of B-3 (500 mg, 1.1 mmol), 1-Boc-piperazine (616 mg, 3.31 mmol), and TEA (768 μL, 5.52 mmol) in DCM (10 mL). The resulting mixture was stirred at room temperature overnight. The reaction mixture was washed with sat. aq. NH₄Cl, dried over Na₂SO₄, filtered, and concentrated. The residue was purified by flash column chromatography (0% to 40% of EtOAc in hexanes) to afford Intermediate B (530 mg, 77% yield). ¹H NMR (600 MHz, MeOD) δ 7.90-7.85 (m, 2H), 7.38-7.34 (m, 2H), 7.14-7.10 (m, 2H), 6.96-6.91 (m, 2H), 3.53-3.40 (m, 4H), 3.37-3.31 (m, 4H), 3.10-3.00 (m, 2H), 2.89-2.72 (m, 1H), 2.67-2.53 (m, 8H), 2.43-2.23 (m, 5H), 2.01 (d, J=17.4 Hz, 1H), 1.72-1.64 (m, 1H), 1.53 (dt, J=11.6, 5.3 Hz, 1H), 1.47 (s, 9H), 1.05 (s, 3H) ppm. LC/MS (ESI) m/z 623.2; [M+H]⁺ calcd for C₃₅H₄₈ClF₃N₄O₄⁺: 623.34.

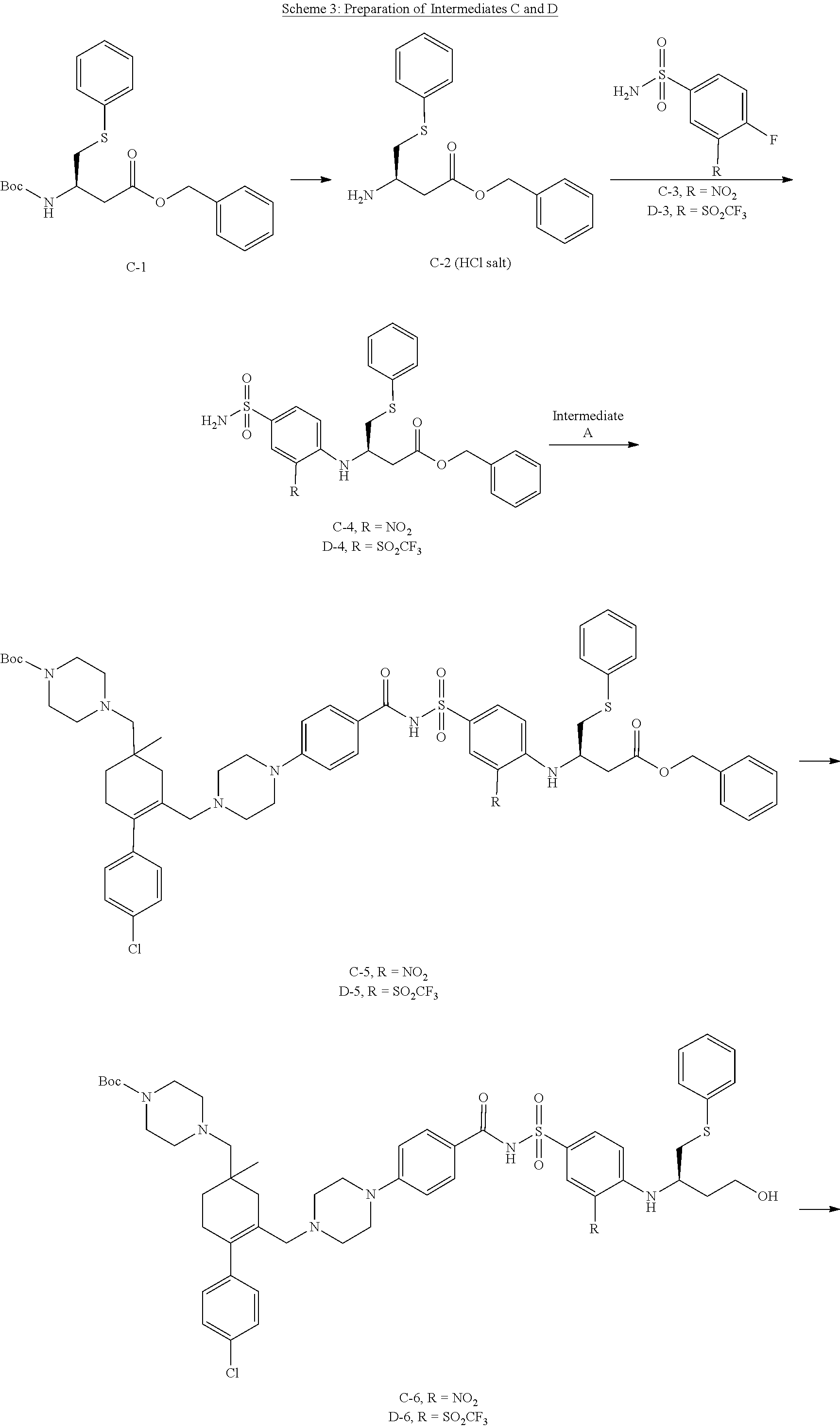

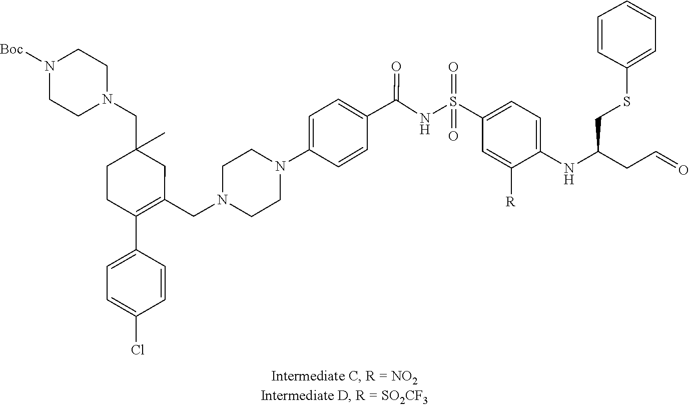

Preparation of benzyl (R)-3-amino-4-(phenylthio)butanoate (C-2): HCl dioxane solution (4N, 10 mL) was added to a solution of C-1 (1.0 g, 2.49 mmol) in DCM (10 mL) with stirring. After stirring at room temperature for 2 hours, the reaction mixture was concentrated to afford crude C-2 (950 mg) as an HCl salt, which was used directly in the next step. LC/MS (ESI) m/z 302.1; [M+H]⁺ calcd for C₁₇H₂₀ClF₃NO₂S⁺: 302.12.

Preparation of benzyl (R)-3-((2-nitro-4-sulfamoylphenyl)amino)-4-(phenylthio)butanoate (C-4) and benzyl (R)-4-(phenylthio)-3-((4-sulfamoyl-2-((trifluoromethyl)sulfonyl)phenyl)amino)butanoate (D-4): C-3 or D-3 (1.0 equiv.) was added to a solution of C-2 (1.0 equiv.) and DIPEA (8.0 equiv.) in DMSO. The resulting mixture was stirred at room temperature overnight. The reaction mixture was diluted with EtOAc and washed with sat. aq. NH₄Cl. The organic layer was dried over Na₂SO₄, filtered, and concentrated. The residue was purified by flash column chromatography to afford C-4 and D-4, respectively. C-4 (140 mg, 56% yield in two steps): ¹H NMR (600 MHz, CDCl₃) δ 8.71-8.65 (m, 2H), 7.74 (dd, J=9.0, 2.3 Hz, 1H), 7.41-7.25 (m, 10H), 6.67 (d, J=9.2 Hz, 1H), 5.15 (q, 2H), 4.82 (s, 2H), 4.31-4.23 (m, 1H), 3.24-3.17 (m, 2H), 2.96 (dd, J=16.3, 5.1 Hz, 1H), 2.87 (dd, J=16.3, 6.8 Hz, 1H) ppm. LC/MS (ESI) m/z 502.1; [M+H]⁺ calcd for C₂₃H₂₄N₃O₆S₂⁺: 502.11. D-4 (1.1 g, 75% yield for two steps): ¹H NMR (600 MHz, CDCl₃) δ 8.26 (d, J=2.2 Hz, 1H), 7.83 (dd, J=9.1, 2.3 Hz, 1H), 7.44-7.39 (m, 2H), 7.39-7.29 (m, 8H), 6.55 (d, J=9.3 Hz, 1H), 5.16 (d, J=12.1 Hz, 1H), 5.11 (d, J=12.1 Hz, 1H), 4.98 (s, 1H), 4.19-4.09 (m, 1H), 3.21-3.07 (m, 2H), 2.90 (dd, J=16.5, 5.0 Hz, 1H), 2.81 (dd, J=16.5, 6.6 Hz, 1H) ppm. LC/MS (ESI) m/z 589.1; [M+H]⁺ calcd for C₂₄H₂₄N₂O₆S₃⁺: 589.07.

Preparation of tert-butyl 4-((6-((4-(4-(((4-(((R)-4-(benzyloxy)-4-oxo-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (C-5) and tert-butyl 4-((6-((4-(4-(((4-(((R)-4-(benzyloxy)-4-oxo-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (D-5): C-4 or D-4 (1.0 equiv.) was added to a mixture of Intermediate A (1.0 equiv.), DMAP (6.0 equiv.) and EDC (6.0 equiv.) in DCM. The resulting mixture was stirred at room temperature overnight. The reaction mixture was washed with water and sat. aq. NH₄Cl. The organic layers were dried over Na₂SO₄, filtered, and concentrated. The residue was purified by flash column chromatography to afford C-5 and D-5, respectively.

C-5 (136 mg, 62% yield): ¹H NMR (600 MHz, CDCl₃) δ 8.81 (s, 1H), 8.71 (d, J=8.9 Hz, 1H), 8.02 (dd, J=9.2, 2.3 Hz, 1H), 7.67 (d, J=8.5 Hz, 2H), 7.37-7.20 (m, 12H), 7.04-6.98 (m, 2H), 6.78 (d, J=8.6 Hz, 2H), 6.65 (d, J=9.3 Hz, 1H), 5.13 (q, 2H), 4.31-4.20 (m, 1H), 3.48-3.38 (m, 4H), 3.28 (t, J=5.2 Hz, 4H), 3.23-3.14 (m, 2H), 2.97-2.82 (m, 4H), 2.51 (s, 4H), 2.44-2.11 (m, 9H), 1.94 (d, J=17.4 Hz, 1H), 1.66-1.58 (m, 1H), 1.52-1.43 (m, 10H), 0.96 (s, 3H) ppm. LC/MS (ESI) m/z 1106.5; [M+H]⁺ calcd for C₅₈H₆₉ClN₇O₉S₂⁺: 1106.43. D-5 (225 22 mg, 65% yield): LC/MS (ESI) m/z 1193.2; [M+H]⁺ calcd for C₅₉H₆₉ClF₃N6O₉S₃⁺: 1193.39

Preparation of tert-butyl 4-((4′-chloro-6-((4-(4-(((4-(((R)-4-hydroxy-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (C-6) and tert-butyl 4-((4′-chloro-6-((4-(4-(((4-(((R)-4-hydroxy-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (D-6): To a solution of C-5 or D-5 (1.0 equiv.) in THF/MeOH (50 mL/2.5 mL) was added NaBH₄ (20.0 equiv.) in portions over a period of 6 hours. The mixture was then stirred at room temperature overnight. Sat. aq. NH₄Cl was added and the mixture was extracted with DCM (×3). The combined organic layers were washed with sat. aq. NH₄Cl, dried over Na₂SO₄, filtered, and concentrated. The residue was purified by flash column chromatography to afford C-6 and D-6, respectively. C-6 (112 mg, 91% yield): ¹H NMR (600 MHz, CDCl₃) δ 8.81 (d, J=2.3 Hz, 1H), 8.63 (d, J=8.6 Hz, 1H), 8.00 (dd, J=9.3, 2.3 Hz, 1H), 7.70-7.63 (m, 2H), 7.39-7.32 (m, 2H), 7.30 (d, J=1.8 Hz, 2H), 7.27-7.19 (m, 3H), 7.03-6.98 (m, 2H), 6.77 (t, J=9.5 Hz, 3H), 4.20-4.09 (m, 1H), 3.89-3.75 (m, 2H), 3.41 (t, J=5.1 Hz, 4H), 3.28 (t, J=5.2 Hz, 4H), 3.20 (qd, J=14.1, 5.8 Hz, 2H), 2.86 (s, 2H), 2.57-2.45 (m, 4H), 2.45-2.11 (m, 11H), 2.01-1.90 (m, 2H), 1.63 (td, J=16.3, 8.8 Hz, 1H), 1.50-1.44 (m, 10H), 0.96 (s, 3H) ppm. LC/MS (ESI) m/z 1002.3; [M+H]⁺ calcd for C₅₁H₆₅ClN₇O₈S₂⁺: 1002.40. D-6 (181 mg, 88% yield): LC/MS (ESI) m/z 1089.4; [M+H]⁺ calcd for C₅₂H₆₅ClF₃N₆O₈S₃⁺: 1089.37.

Preparation of tert-butyl 4-((4′-chloro-4-methyl-6-((4-(4-(((3-nitro-4-(((R)-4-oxo-1-(phenylthio)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (Intermediate C) and tert-butyl 4-((4′-chloro-4-methyl-6-((4-(4-(((4-(((R)-4-oxo-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (Intermediate D): DMP (2.0 equiv.) was added to a solution of compound C-6 or D-6 (1.0 equiv.) in EtOAc, then stirred at room temperature for 3 hours. The reaction mixture was filtered, and the filtrate was concentrated to afford crude Intermediate C and Intermediate D, respectively, which were used directly in the next step or purified by flash column chromatography. Intermediate C (84 mg, 75% yield): LC/MS (ESI) m/z 1000.4; [M+H]⁺ calcd for C₅₁H₆₃ClN₇O₈S₂⁺: 1000.39. Intermediate D (155 mg, 86% yield): LC/MS (ESI) m/z 1087.5; [M+H]⁺ calcd for C₅₂H₆₃ClF₃N₆O₈S₃⁺: 1087.35.

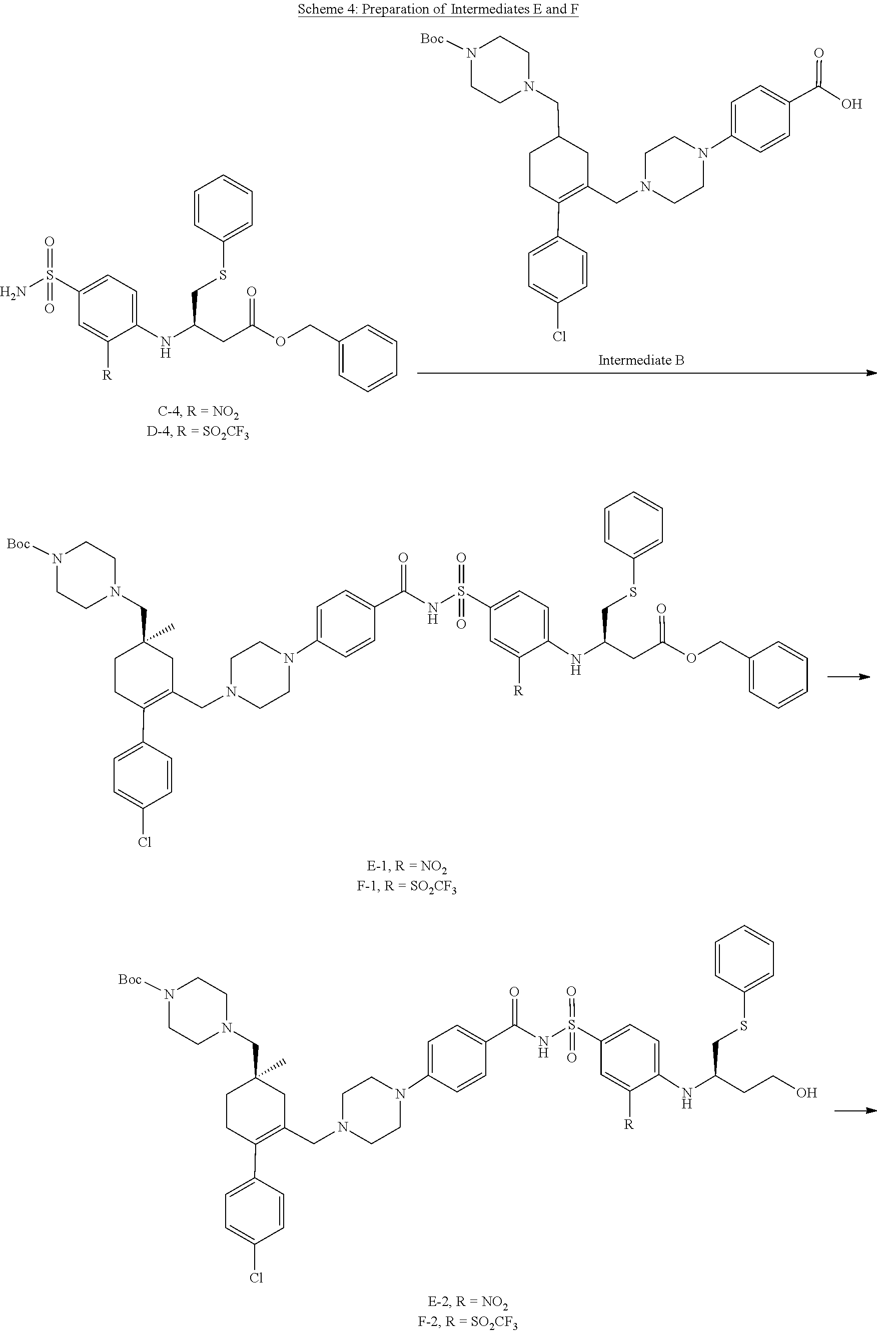

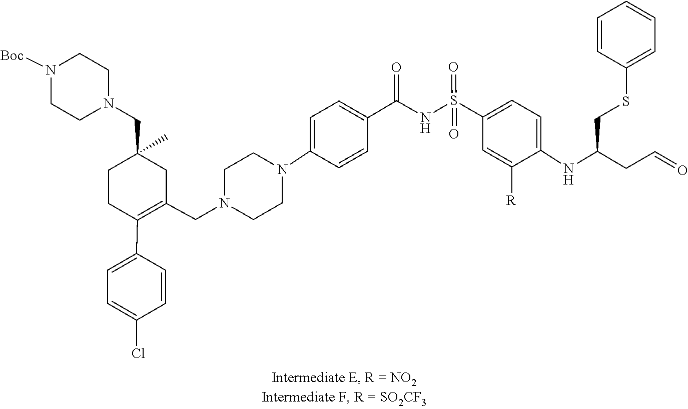

Preparation of tert-butyl 4-(((R)-6-((4-(4-(((4-(((R)-4-(benzyloxy)-4-oxo-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (E-1) and tert-butyl 4-(((R)-6-((4-(4-(((4-(((R)-4-(benzyloxy)-4-oxo-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (F-1): C-4 or D-4 (1.0 equiv.) was added to a mixture of Intermediate B (1.0 equiv.), DMAP (6.0 equiv.) and EDC (6.0 equiv.) in DCM. The resulting mixture was stirred at room temperature overnight. The reaction mixture was washed with water and sat. aq. NH₄Cl. The organic layers were dried over Na₂SO₄, filtered, and concentrated. The residue was purified by flash column chromatography to afford E-1 and F-1, respectively. E-1 (150 mg, 84% yield): LC/MS (ESI) m/z 1106.5; [M+H]⁺ calcd for C₅₈H₆₉ClN₇O₉S₂⁺: 1106.43. F-1 (220 mg, 57% yield): ¹H NMR (600 MHz, Chloroform-d) δ 8.39 (d, J=2.3 Hz, 1H), 8.14 (dd, J=9.2, 2.3 Hz, 1H), 7.66 (d, J=8.7 Hz, 2H), 7.42-7.37 (m, 3H), 7.38-7.26 (m, 10H), 7.04-6.97 (m, 2H), 6.79 (d, J=8.7 Hz, 2H), 6.52 (d, J=9.4 Hz, 1H), 5.19-5.06 (m, 2H), 4.17-4.07 (m, 1H), 3.46-3.38 (m, 4H), 3.29 (t, J=5.3 Hz, 4H), 3.20-3.06 (m, 2H), 2.93-2.76 (m, 4H), 2.52 (s, 4H), 2.45-2.34 (m, 4H), 2.34-2.12 (m, 5H), 1.95 (d, J=17.3 Hz, 1H), 1.66-1.58 (m, 1H), 1.47 (s, 10H), 0.96 (s, 3H) ppm. LC/MS (ESI) m/z 1193.4; [M+H]⁺ calcd for C₅₉H₆₉ClF₃N₆O₉S₃⁺: 1193.39.

Preparation of tert-butyl 4-(((R)-4′-chloro-6-((4-(4-(((4-(((R)-4-hydroxy-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (E-2) and tert-butyl 4-(((R)-4′-chloro-6-((4-(4-(((4-(((R)-4-hydroxy-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (F-2): To a solution of E-1 or F-1 (1.0 equiv.) in THF/MeOH (50 mL/2.5 mL) was added NaBH₄ (20.0 equiv.) in portions over a period of 6 hours, then stirred at room temperature overnight. The reaction mixture was added sat. aq. NH₄Cl and extracted with DCM. The combined organic layers were washed with sat. aq. NH₄Cl, then dried over Na₂SO₄, filtered, and concentrated. The residue was purified by flash column chromatography to afford E-2 and F-2, respectively. E-2 (99 mg, 73% yield): LC/MS (ESI) m/z 1002.3; [M+H]⁺ calcd for C₅₁H₆₅ClN₇O₈S₂⁺: 1002.40. F-2 (168 mg, 66% yield). ¹H NMR (600 MHz, Chloroform-d) δ 8.36 (s, 1H), 8.10 (s, 1H), 7.76-7.62 (m, 2H), 7.39 (d, J=7.6 Hz, 2H), 7.34-7.17 (m, 7H), 7.00 (d, J=8.2 Hz, 2H), 6.75 (s, 2H), 6.60 (d, J=9.3 Hz, 1H), 4.01 (s, 1H), 3.75 (d, J=36.2 Hz, 2H), 3.40 (s, 5H), 3.28 (s, 4H), 3.18-3.05 (m, 2H), 3.01-2.82 (m, 2H), 2.57-2.36 (m, 9H), 2.36-2.08 (m, 5H), 1.96 (d, J=17.4 Hz, 1H), 1.85 (s, 1H), 1.68-1.57 (m, 1H), 1.46 (s, 9H), 0.96 (s, 3H). LC/MS (ESI) m/z 1089.5; [M+H]⁺ calcd for C₅₂H₆₅ClF₃N₆O₈S₃⁺: 1089.37.

Preparation of tert-butyl 4-(((4R)-4′-chloro-4-methyl-2-((4-(4-(((3-nitro-4-(((R)-4-oxo-1-(phenylthio)butan-2-yl)amino)phenyl)sulfonyl)carbamoyl)phenyl)piperazin-1-yl)methyl)-[1,1′-bi(cyclohexane)]-1,1′,5′-trien-4-yl)methyl)piperazine-1-carboxylate (Intermediate E) and tert-Butyl 4-(((4R)-4′-chloro-4-methyl-2-((4-(4-(((4-(((R)-4-oxo-1-(phenylthio)butan-2-yl)amino)-3-((trifluoromethyl)sulfonyl)phenyl)sulfonyl)carbamoyl)phenyl)piperazin-1-yl)methyl)-[1,1′-bi(cyclohexane)]-1,1′,5′-trien-4-yl)methyl)piperazine-1-carboxylate (Intermediate F): DMP (2.0 equiv.) was added to a solution of compound E-2 or F-2 (1.0 equiv.) in EtOAc, then stirred at room temperature for 3 hours. The reaction mixture was filtered, and the filtrate was concentrated to afford crude intermediate E and intermediate F, which were used directly in the next step or purified by flash column chromatography. Intermediate E (171 16 mg, crude product): LC/MS (ESI) m/z 1000.3; [M+H]⁺ calcd for C₅₁H₆₃ClN₇O₈S₂⁺: 1000.39. Intermediate F (90 mg, 55% yield): ¹H NMR (600 MHz, Chloroform-d) δ 9.74 (d, J=2.6 Hz, 1H), 8.40 (dd, J=5.2, 2.3 Hz, 1H), 8.19 (td, J=9.5, 8.7, 2.3 Hz, 1H), 7.66 (dd, J=12.2, 8.6 Hz, 2H), 7.44-7.39 (m, 2H), 7.37-7.22 (m, 6H), 7.03-6.98 (m, 2H), 6.77 (dd, J=19.9, 8.7 Hz, 2H), 6.58 (dd, J=9.4, 2.6 Hz, 1H), 4.26-4.17 (m, 1H), 3.42 (d, J=5.9 Hz, 4H), 3.35-3.26 (m, 4H), 3.21 (dd, J=14.2, 5.0 Hz, 1H), 3.16-3.09 (m, 1H), 3.07-2.97 (m, 2H), 2.97-2.89 (m, 2H), 2.64-2.41 (m, 9H), 2.37-2.15 (m, 5H), 2.05-1.93 (m, 1H), 1.70-1.58 (m, 1H), 1.52-1.42 (m, 10H), 0.97 (s, 3H) ppm. LC/MS (ESI) m/z 1087.4; [M+H]⁺ calcd for C₅₂H₆₃ClF₃N₆O₈S₃⁺: 1087.35.

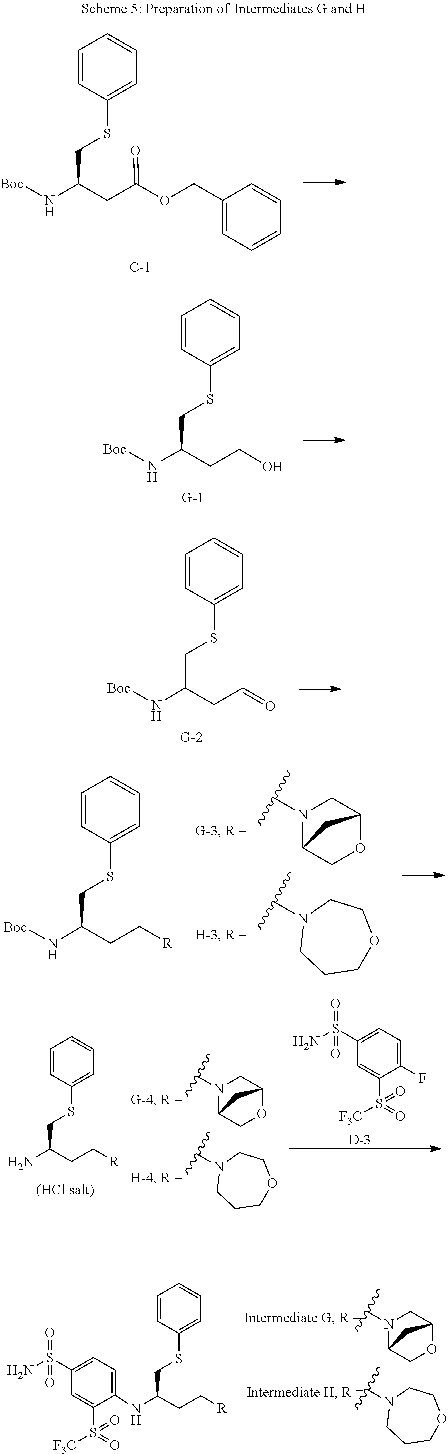

Preparation of tert-butyl (R)-(4-hydroxy-1-(phenylthio)butan-2-yl)carbamate (G-1): To a solution of C-1 (2.7 g, 6.724 mmol) in THF/MeOH (50 mL/5 mL) was added NaBH₄ (1.49 g, 40.35 mmol) in portions over a period of 6 hours. The resulting mixture was then stirred at room temperature overnight. Sat. aq. NH₄Cl was added and the mixture was extracted with DCM (×3).

The combined organic layers were washed with sat. aq. NH₄Cl, dried over Na₂SO₄, filtered, and concentrated. The residue was purified by flash column chromatography (0% to 70% of EtOAc in hexanes) to afford G-1 (1.6 g, 80% yield). ¹H NMR (600 MHz, Chloroform-d) δ (ppm): 7.43-7.40 (m, 2H), 7.32 (t, J=7.8 Hz, 2H), 7.26-7.21 (m, 1H), 4.84 (d, J=8.4 Hz, 1H), 4.07 (s, 1H), 3.67 (s, 2H), 3.22-2.98 (m, 3H), 1.98-1.82 (m, 1H), 1.67-1.49 (m, 1H), 1.46 (s, 9H) ppm. LC/MS (ESI) m/z 298.1; [M+H]⁺ calcd for C₁₅H₂₄NO₃S⁺: 298.15.

Preparation of tert-butyl (R)-(4-oxo-1-(phenylthio)butan-2-yl)carbamate (G-2): DMP (2.08 g, 4.90 mmol) was added to a solution of G-1 (1.08 g, 3.63 mmol) in EtOAc (25 mL) and stirred at room temperature for 3 hours. The reaction mixture was filtered and the filtrate was concentrated. The residue was purified by flash column chromatography (0% to 70% of EtOAc in hexanes) to afford G-2 (1.0 g, 93% yield). ¹H NMR (600 MHz, Chloroform-d) δ 9.71 (s, 1H), 7.43-7.40 (m, 2H), 7.35-7.30 (m, 2H), 7.26-7.21 (m, 1H), 5.00 (s, 1H), 4.22 (s, 1H), 3.33-3.21 (m, 1H), 3.17-3.07 (m, 1H), 2.90-2.72 (m, 2H), 1.44 (s, 9H) ppm. LC/MS (ESI) m/z 296.1; [M+H]⁺ calcd for C₁₅H₂₂NO₃S⁺: 296.13.

Preparation of tert-butyl ((R)-4-((1R,4R)-2-oxa-5-azabicyclo[2.2.1]heptan-5-yl)-1-(phenylthio)butan-2-yl)carbamate (G-3) and tert-butyl (R)-(4-(1,4-oxazepan-4-yl)-1-(phenylthio)butan-2-yl)carbamate (H-3): NaBH(OAc)₃ (1.2 equiv.) was added to a solution of G-2 (1 equiv.), and amines (1.1 equiv.) in DCM, then stirred at room temperature overnight. The reaction mixture was washed with sat. aq. NH₄Cl. The organic layers were dried over Na₂SO₄, filtered, and concentrated. The residue was purified by flash column chromatography (50% to 100% of EtOAc in hexanes, then 0% to 10% of MeOH in DCM) to afford G-3 and H-3, respectively. G-3 (360 mg, 56%): ¹H NMR (600 MHz, Methanol-d₄) δ 7.44 (d, 2H), 7.33 (t, J=7.8 Hz, 2H), 7.25-7.20 (m, 1H), 4.60 (s, 1H), 4.14 (s, 1H), 4.07 (d, 1H), 3.81-3.68 (m, 2H), 3.21-3.09 (m, 4H), 3.09-2.97 (m, 2H), 2.17-2.01 (m, 3H), 1.84-1.72 (m, 1H), 1.46 (s, 9H). LC/MS (ESI) m/z 379.2; [M+H]⁺ calcd for C₂₀H₃₁N₂O₃S⁺: 379.20. H-3 (760 mg, 98% yield): ¹H NMR (600 MHz, Chloroform-d) δ 7.42 (d, J=7.6 Hz, 2H), 7.33-7.29 (m, 2H), 7.19 (t, J=7.4 Hz, 1H), 3.91 (s, 1H), 3.82 (t, J=6.1 Hz, 2H), 3.73 (t, 2H), 3.30 (d, J=13.5 Hz, 1H), 3.07-2.95 (m, 1H), 2.77-2.60 (m, 4H), 2.59-2.52 (m, 1H), 1.95-1.82 (m, 3H), 1.74-1.66 (m, 1H), 1.45 (s, 9H). LC/MS (ESI) m/z 381.4; [M+H]⁺ calcd for C₂₀H₃₃N₂O₃S⁺: 381.22.

Preparation of (R)-4-((1R,4R)-2-oxa-5-azabicyclo[2.2.1]heptan-5-yl)-1-(phenylthio)butan-2-amine hydrochloride (G-4) and (R)-4-(1,4-oxazepan-4-yl)-1-(phenylthio)butan-2-amine (H-4): HCl dioxane solution (4N) was added to a solution of G-3 or H-3 in DCM, then stirred at room temperature for 1 hour, the reaction mixture was concentrated to afford G-4 and H-4, respectively, which was used directly in the next step. G-4 (350 mg, quantitative yield): LC/MS (ESI) m/z 279.1; [M+H]⁺ calcd for C₁₅H₂₃N₂OS⁺: 279.15. H-4 (750 mg, quantitative yield). LC/MS (ESI) m/z 281.2; [M+H]⁺ calcd for C₁₅H₂₅N₂OS⁺: 281.17.

Preparation of 4-(((R)-4-((1R,4R)-2-oxa-5-azabicyclo[2.2.1]heptan-5-yl)-1-(phenylthio)butan-2-yl)amino)-3-((trifluoromethyl)sulfonyl)benzenesulfonamide (Intermediate G) and (R)-4-((4-(1,4-oxazepan-4-yl)-1-(phenylthio)butan-2-yl)amino)-3-((trifluoromethyl)sulfonyl)benzenesulfonamide (Intermediate H): D-3 (1.0 equiv.) was added to a solution of G-4 or H-4 (1.2 equiv.) and DIPEA (8.0 equiv.) in DMSO, then stirred at room temperature overnight. The reaction mixture was diluted with EtOAc and washed with sat. aq. NH₄Cl. The organic layer was dried over Na₂SO₄, filtered, and concentrated. The residue was purified by flash column chromatography (50% to 100% of EtOAc in hexanes, then 0% to 10% of MeOH in DCM) to afford Intermediate G or Intermediate H, respectively. Intermediate G (480 mg, 89% yield in two steps): ¹H NMR (600 MHz, Methanol-d₄) δ 8.15 (d, J=2.2 Hz, 1H), 7.88 (dd, J=9.2, 2.3 Hz, 1H), 7.45-7.38 (m, 2H), 7.34-7.28 (m, 2H), 7.28-7.22 (m, 1H), 6.89 (d, J=9.3 Hz, 1H), 4.40 (s, 1H), 4.14-4.05 (m, 1H), 3.95 (d, J=7.9 Hz, 1H), 3.60 (dd, J=7.8, 1.8 Hz, 1H), 3.48 (s, 1H), 3.30 (dd, 1H), 3.19 (dd, J=14.2, 5.9 Hz, 1H), 2.80 (dd, J=10.3, 1.8 Hz, 1H), 2.75-2.60 (m, 2H), 2.54 (dd, J=10.2, 1.5 Hz, 1H), 2.06-1.94 (m, 1H), 1.87-1.75 (m, 2H), 1.75-1.69 (m, 1H) ppm. LC/MS (ESI) m/z 566.1; [M+H]⁺ calcd for C₂₂H₂₇F₃N₃O₅S₃⁺: 566.11. Intermediate H (900 mg, 83% yield in two steps). ¹H NMR (600 MHz, Chloroform-d) δ 8.27 (d, J=2.2 Hz, 1H), 7.83 (dd, J=9.2, 2.3 Hz, 1H), 7.44-7.40 (m, 2H), 7.37-7.33 (m, 2H), 7.33-7.27 (m, 1H), 7.19 (d, J=8.5 Hz, 1H), 6.65 (d, J=9.2 Hz, 1H), 5.06 (s, 2H), 4.01-3.91 (m, 1H), 3.77 (t, J=6.0 Hz, 2H), 3.73-3.63 (m, 2H), 3.17-3.03 (m, 2H), 2.75-2.69 (m, 2H), 2.68-2.52 (m, 4H), 2.14-2.04 (m, 1H), 1.90-1.82 (m, 2H), 1.76-1.67 (m, 1H) ppm. LC/MS (ESI) m/z 568.0; [M+H]⁺ calcd for C₂₂H₂₉F₃N₃O₅S₃⁺: 568.12.

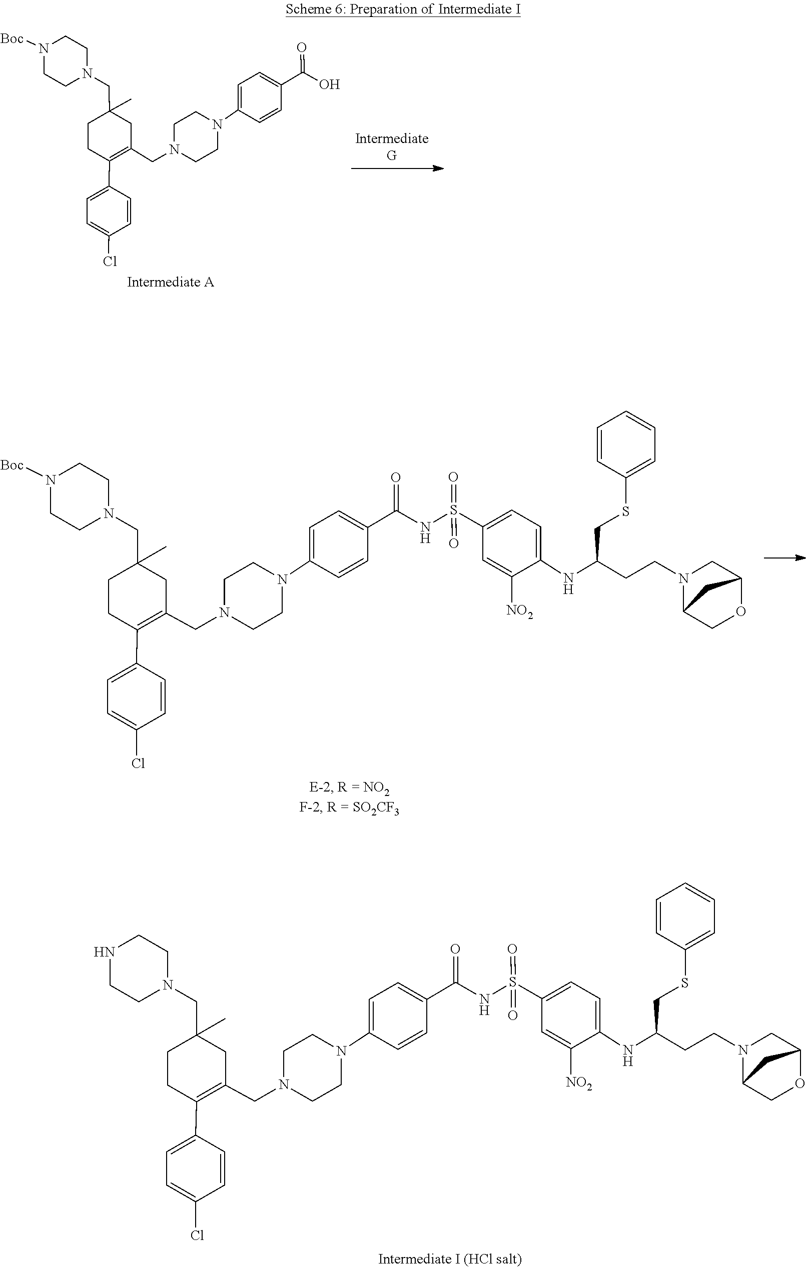 [SMILES: CC1(CN2CCN(C(=O)OC(C)(C)C)CC2)CCC(c2ccc(Cl)cc2)=C(CN2CCN(c3ccc(C(=O)O)cc3)CC2)C1 ; CC1(CN2CCN(C(=O)OC(C)(C)C)CC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c([N+](=O)[O-])c4)cc3)CC2)C1 ; CC1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c([N+](=O)[O-])c4)cc3)CC2)C1]

Preparation of tert-butyl 4-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (I-1): Intermediate G (36 mg, 0.064 mmol) was added to a mixture of Intermediate A (40 mg, 0.064 mmol), EDC (62 mg, 0.32 mmol) and DMAP (39 mg, 0.32 mmol) in DCM (3 mL). The resulting reaction mixture was stirred at room temperature overnight. The reaction mixture was washed with water and sat. aq. NH₄Cl, dried over Na₂SO₄, filtered, and concentrated. The residue was purified by flash column chromatography (0% to 10% of MeOH in DCM) to afford 1-1 (39 12 mg, 52% yield). ¹H NMR (600 MHz, Chloroform-d) δ 8.34 (s, 1H), 8.03 (d, J=8.8, 2.5 Hz, 1H), 7.79-7.74 (m, 2H), 7.42-7.35 (m, 2H), 7.34-7.22 (m, 5H), 7.06-6.97 (m, 3H), 6.78 (d, J=8.6 Hz, 2H), 6.57 (d, J=9.3 Hz, 1H), 4.46 (s, 1H), 4.03 (d, J=8.3 Hz, 1H), 3.97-3.89 (m, 1H), 3.73-3.62 (m, 2H), 3.42 (t, J=4.8 Hz, 4H), 3.28 (t, J=5.2 Hz, 4H), 3.13-2.98 (m, 3H), 2.94-2.79 (m, 3H), 2.78-2.65 (m, 2H), 2.56-2.47 (m, 4H), 2.42 (s, 4H), 2.36-2.04 (m, 6H), 2.03-1.91 (m, 2H), 1.86-1.73 (m, 2H), 1.61 (qd, J=12.0, 10.9, 8.0 Hz, 1H), 1.51-1.41 (m, 10H), 0.96 (d, J=3.3 Hz, 3H) ppm. LC/MS (ESI) m/z 1170.4; [M+H]⁺ calcd for Cs₇H₇₂ClF₃N₇O₈S₃⁺: 1170.42.

Preparation of N-((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)-4-(4-((4′-chloro-4-methyl-4-(piperazin-1-ylmethyl)-3,4,5,6-tetrahydro-[1,1′-biphenyl]-2-yl)methyl)piperazin-1-yl)benzamide hydrochloride (Intermediate I): HCl dioxane solution (4N, 1 mL) was added to a solution of compound 1-1 (39 mg, 0.033 mmol) in DCM (1 mL). After stirring at room temperature for 2 hours, the reaction mixture was concentrated to afford crude Intermediate I (37 mg, quantitative yield), which was used directly in the next step. LC/MS (ESI) m/z 1070.4; [M+H]⁺ calcd for C₅₂H₆₄ClF₃N₇O₆S₃⁺: 1070.37.

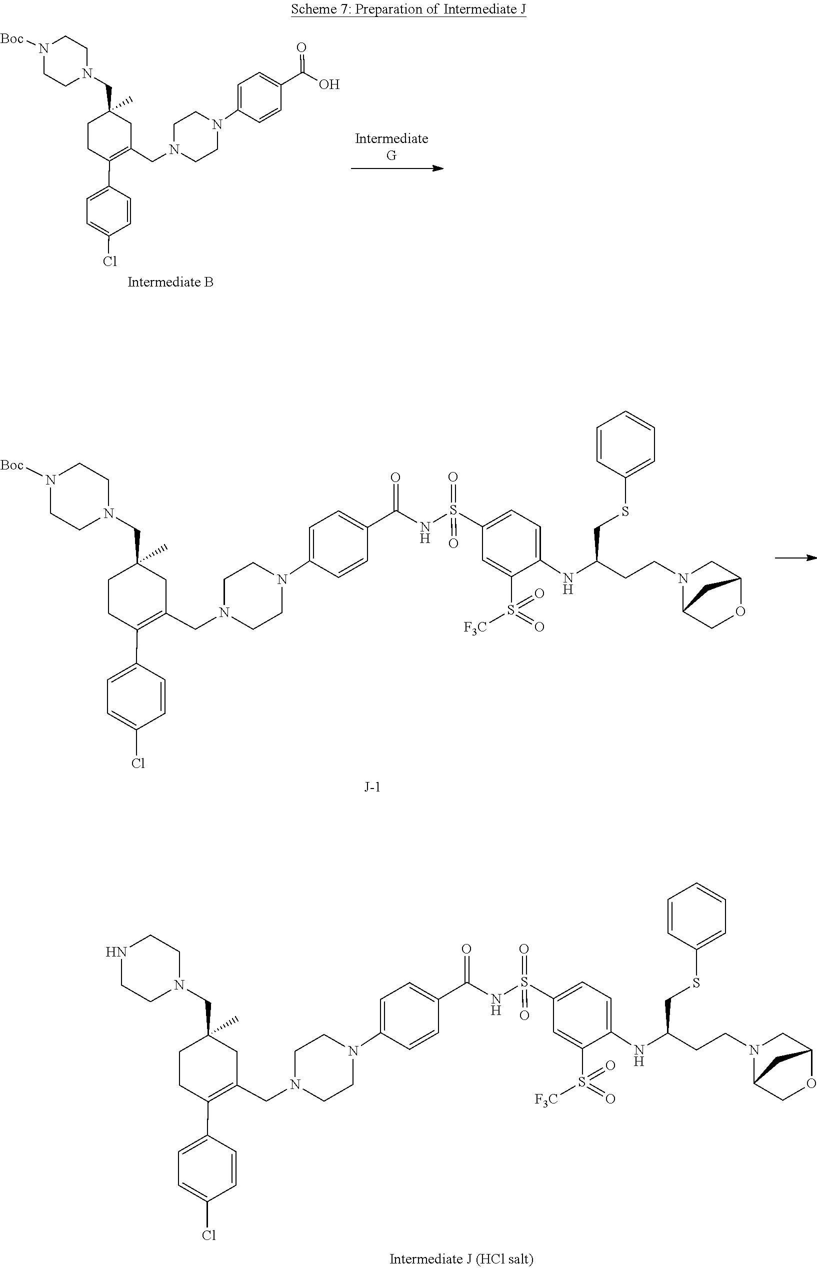 [SMILES: CC(C)(C)OC(=O)N1CCN(C[C@]2(C)CCC(c3ccc(Cl)cc3)=C(CN3CCN(c4ccc(C(=O)O)cc4)CC3)C2)CC1 ; CC(C)(C)OC(=O)N1CCN(C[C@]2(C)CCC(c3ccc(Cl)cc3)=C(CN3CCN(c4ccc(C(=O)NS(=O)(=O)c5ccc(N[C@H](CCN6C[C@H]7C[C@@H]6CO7)CSc6ccccc6)c(S(=O)(=O)C(F)(F)F)c5)cc4)CC3)C2)CC1 ; C[C@@]1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c(S(=O)(=O)C(F)(F)F)c4)cc3)CC2)C1]

Preparation of tert-butyl 4-(((R)-6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (J-1): Intermediate G (91 mg, 0.16 mmol) was added to a mixture of intermediate A (100 mg, 0.16 mmol), EDC (154 mg, 0.8 mmol) and DMAP (98 mg, 0.8 mmol) in DCM (7 mL). The resulting mixture was stirred at room temperature overnight. The reaction mixture was washed with water and sat. aq. NH₄Cl. The organic layer was dried over Na₂SO₄, filtered, and concentrated. The residue was purified by flash column chromatography (0% to 10% of MeOH in DCM) to afford I-1 (157 mg, 84% yield). ¹H NMR (600 MHz, Chloroform-d) δ 8.35 (t, J=2.2 Hz, 1H), 8.09-8.02 (m, 1H), 7.76-7.71 (m, 2H), 7.44-7.37 (m, 2H), 7.36-7.22 (m, 5H), 7.14-7.04 (m, 1H), 7.03-6.98 (m, 2H), 6.79 (d, 2H), 6.58 (d, J=9.5, 2.4 Hz, 1H), 4.45 (s, 1H), 4.05-3.98 (m, 1H), 3.98-3.90 (m, 1H), 3.72-3.56 (m, 2H), 3.42 (t, J=4.9 Hz, 4H), 3.28 (t, J=5.2 Hz, 4H), 3.16-3.01 (m, 2H), 3.01-2.92 (m, 1H), 2.90-2.76 (m, 3H), 2.74-2.60 (m, 2H), 2.57-2.46 (m, 4H), 2.46-2.36 (m, 4H), 2.35-2.11 (m, 5H), 2.10-2.01 (m, 1H), 1.99-1.88 (m, 2H), 1.85-1.71 (m, 2H), 1.69-1.55 (m, 1H), 1.53-1.41 (m, 10H), 0.96 (s, 3H) ppm. LC/MS (ESI) m/z 1170.5; [M+H]⁺ calcd for C₅₇H₇₂ClF₃N₇O₈S₃⁺: 1170.42.

Preparation of N-((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (Intermediate J): HCl dioxane solution (4 6N, 4 mL) was added to a solution of J-1 (100 mg, 0.086 mmol) in DCM (4 mL). After stirring at room temperature for 2 hours, the reaction mixture was concentrated and the residue was triturated with diethyl ether, then dried under vacuum to afford Intermediate J (103 mg, quantitative yield), which was used directly in the next step. LC/MS (ESI) m/z 1070.4; [M+H]+ calcd for C₅₂H₆₄ClF₃N₇O₆S₃⁺: 1070.37.

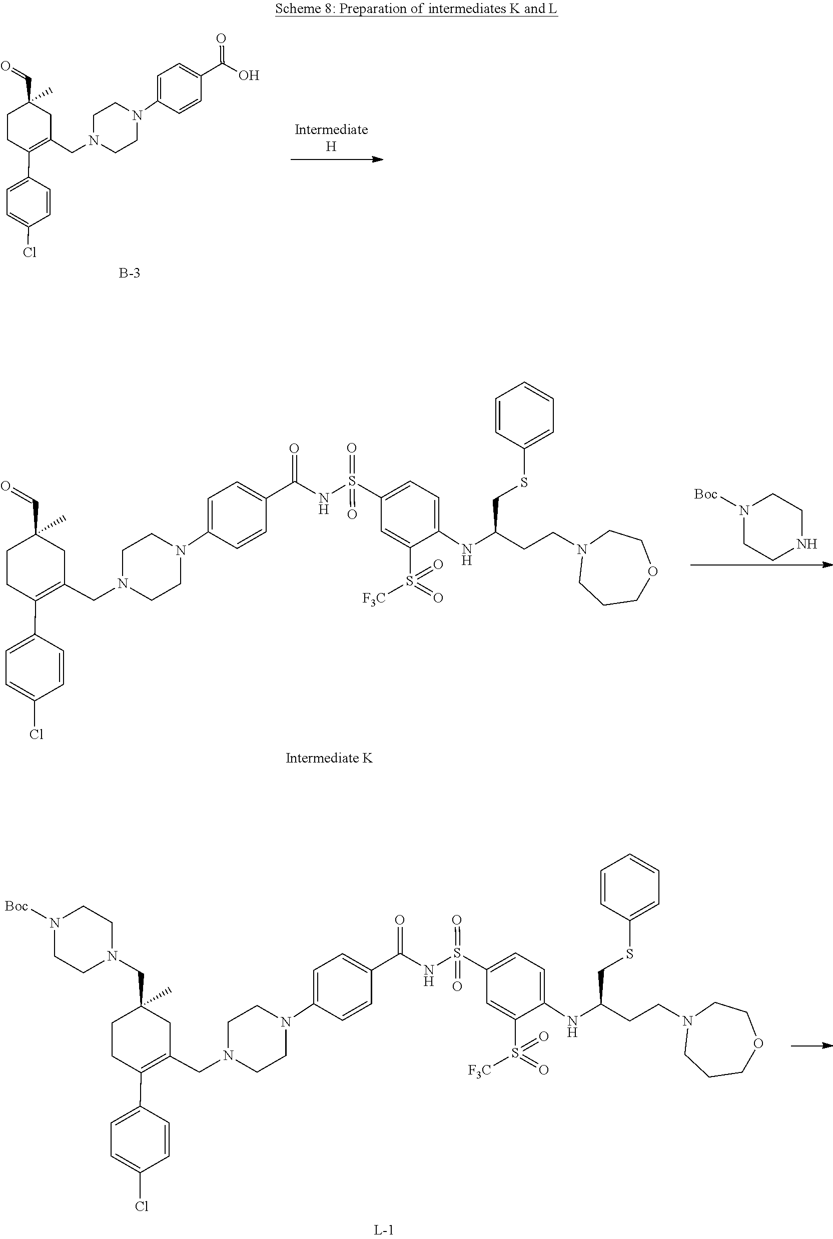

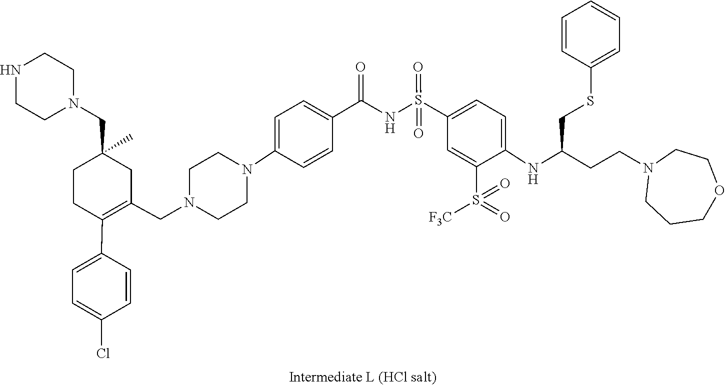 [SMILES: C[C@@]1(CN2CCNCC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Preparation of N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-formyl-4-methyl-3,4,5,6-tetrahydro-[1,1′-biphenyl]-2-yl)methyl)piperazin-1-yl)benzamide (Intermediate K): Intermediate H (250 mg, 0.44 mmol) was added to a mixture of B-3 (200 mg, 0.44 mmol), EDC (424 mg, 2.21 mmol) and DMAP (269 mg, 2.21 mmol) in DCM (10 mL). The resulting mixture was stirred at room temperature overnight. The reaction mixture was washed with water and sat. aq. NH₄Cl, dried over Na₂SO₄, filtered, and concentrated. The residue was purified by flash column chromatography (0% to 10% of MeOH in DCM) to afford Intermediate K (290 mg, 66% yield). ¹H NMR (600 MHz, CDCl₃) δ 9.54 (s, 1H), 8.37 (d, J=2.3 Hz, 1H), 8.08 (dd, J=9.2, 2.3 Hz, 1H), 7.71 (d, J=8.5 Hz, 2H), 7.39 (d, J=7.3 Hz, 2H), 7.37-7.23 (m, 5H), 7.15 (d, J=8.9 Hz, 1H), 6.99-6.96 (m, 2H), 6.80 (d, J=8.6 Hz, 2H), 6.61 (d, J=9.3 Hz, 1H), 3.93 (s, 1H), 3.82-3.66 (m, 4H), 3.35-3.26 (m, 4H), 3.16-2.99 (m, 2H), 2.94-2.58 (m, 8H), 2.51-2.25 (m, 7H), 2.15-1.87 (m, 6H), 1.86-1.72 (m, 1H), 1.69-1.59 (m, 1H), 1.16 (s, 3H) ppm. LC/MS (ESI) m/z 1002.3; [M+H]⁺ calcd for C₄₈H₅₆ClF₃N₅O₇S₃⁺: 1002.30.

Preparation of tert-butyl 4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (L-1): NaBH(OAc)₃ (106 mg, 0.5 mmol) was added to a solution of Intermediate K (250 mg, 0.25 mmol), 1-Boc-piperazine (231 mg, 1.25 mmol) and TEA (208 μL, 1.5 mmol) in DCM (6 mL), then stirred at room temperature overnight. The reaction mixture was washed with sat. aq. NH₄Cl. The organic layer was dried over Na₂SO₄, filtered, and concentrated. The residue was purified by flash column chromatography (0% to 10% of MeOH in DCM) to afford L-1 (240 mg, 82% yield). ¹H NMR (600 MHz, CDCl₃) δ 8.37 (d, J=2.3 Hz, 1H), 8.08 (dd, J=9.3, 2.3 Hz, 1H), 7.71 (d, 2H), 7.41-7.37 (m, 2H), 7.35-7.24 (m, 5H), 7.15 (d, J=8.5 Hz, 1H), 7.01 (d, 2H), 6.81-6.76 (m, 2H), 6.60 (d, J=9.3 Hz, 1H), 3.93 (d, J=9.4 Hz, 1H), 3.83-3.67 (m, 4H), 3.42 (s, 4H), 3.35-3.24 (m, 4H), 3.14-3.01 (m, 2H), 2.90-2.59 (m, 8H), 2.52 (s, 4H), 2.45-2.35 (m, 4H), 2.34-2.20 (m, 4H), 2.19-2.07 (m, 2H), 2.01-1.88 (m, 4H), 1.81-1.70 (m, 1H), 1.67-1.57 (m, 1H), 1.48 (s, 9H), 0.96 (s, 3H) ppm. LC/MS (ESI) m/z 1172.5; [M+H]⁺ calcd for C₅₇H₇₄ClF₃N₇O₈S₃⁺: 1172.44.

Preparation of N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (Intermediate L): HCl dioxane solution (4N, 4 mL) was added to a stirring solution of L-1 (240 mg, 0.204 mmol) in DCM (4 mL). After stirring at room temperature for 2 hours, the reaction mixture was concentrated. The residue was triturated with diethyl ether and dried under vacuum to afford Intermediate L (227 mg, quantitative yield), which was used directly in the next step. LC/MS (ESI) m/z 1072.5; [M+H]⁺ calcd for C₅₂H₆₆ClF₃N₇O₆S₃⁺: 1072.39.

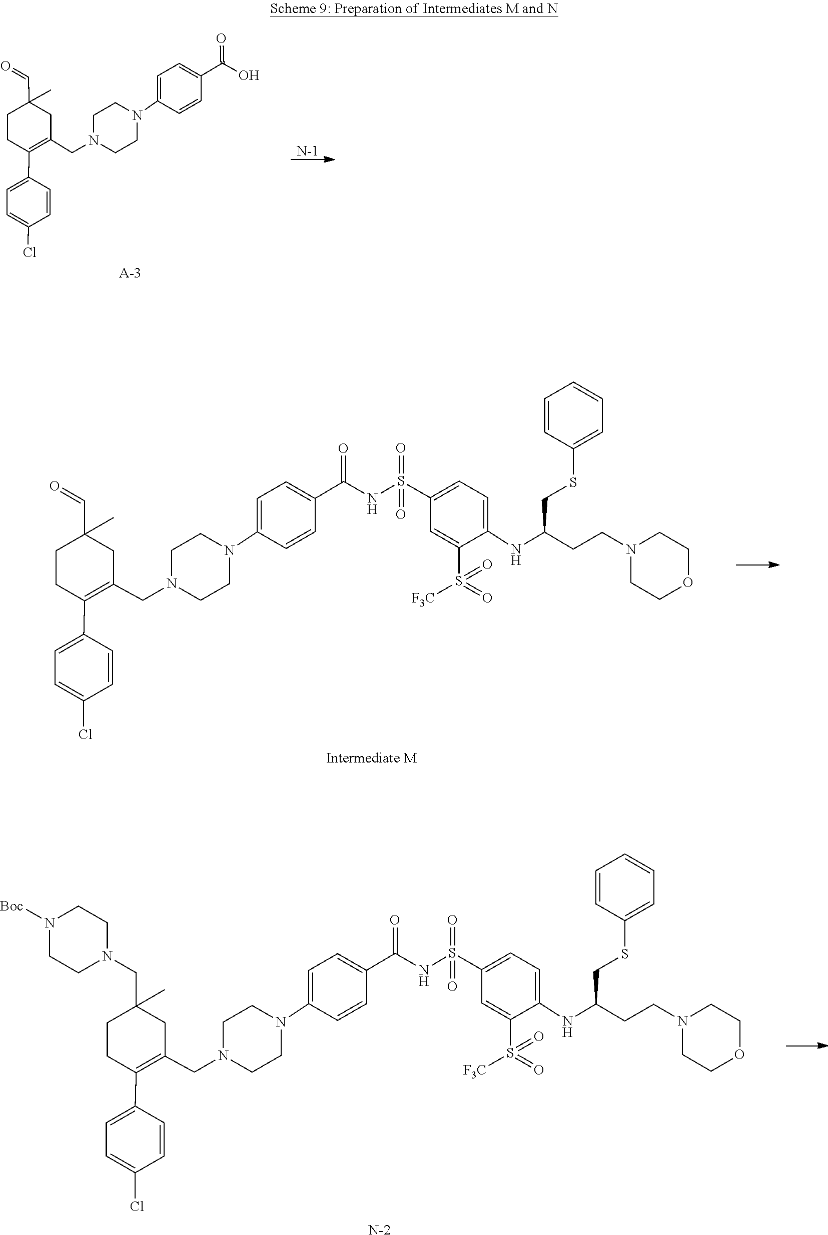

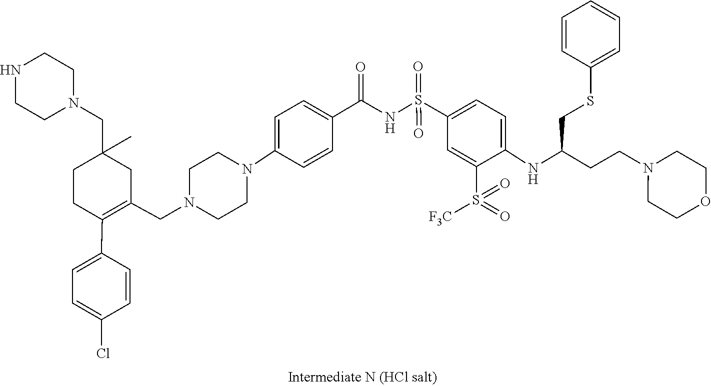 [SMILES: CC1(CN2CCNCC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

Preparation of 4-(4-((4′-chloro-4-formyl-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (Intermediate M): N-1 (1.36 g, 2.45 mmol) was added to a mixture of A-3 (1.2 g, 2.65 mmol), DMAP (1.5 g, 12.27 mmol), and EDC (2.35 g, 12.27 mmol) in DCM (50 mL). The resulting mixture was stirred at room temperature overnight. The reaction mixture was washed with water and sat. aq. NH₄Cl. The organic layers were dried over Na₂SO₄, filtered, and concentrated. The residue was purified by flash column chromatography (0% to 5% of MeOH in DCM) to afford Intermediate M (1.9 g, 72% yield). ¹H NMR (600 MHz, Chloroform-d) δ 9.54 (d, J=1.5 Hz, 1H), 8.38 (d, J=2.3 Hz, 1H), 8.13 (dd, J 12=9.3, 2.4 Hz, 1H), 7.68-7.62 (m, 2H), 7.41-7.37 (m, 2H), 7.36-7.26 (m, 4H), 7.10 (d, J=8.7 Hz, 1H), 6.98-6.94 (m, 2H), 6.81 (d, J=8.9 Hz, 2H), 6.63 (d, J=9.5 Hz, 1H), 3.93 (s, 1H), 3.71-3.61 (m, 6H), 3.32 (t, J=5.2 Hz, 4H), 3.12 (dd, J=13.9, 5.1 Hz, 1H), 3.04 (dd, J=13.9, 7.3 Hz, 1H), 2.88 (s, 2H), 2.69 (d, J=17.6 Hz, 1H), 2.53-2.27 (m, 10H), 2.19-2.10 (m, 1H), 2.09-1.97 (m, 2H), 1.74-1.59 (m, 2H), 1.16 (d, J=2.1 Hz, 3H) ppm. LC/MS (ESI) m/z 988.3; [M+H]⁺ calcd for C₄₇H₅₄ClF₃N₅O₇S₃⁺: 988.28.

Preparation of tert-butyl 4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (N-2): NaBH(OAc)₃ (113 mg, 0.53 mmol) was added partly to a solution of Intermediate M (350 mg, 0.35 mmol), 1-Boc-piperazine (79 mg, 0.43 mmol) and TEA (246 μL, 1.77 mmol) in DCM (6 mL), then stirred at room temperature overnight. The reaction mixture was washed with sat. aq. NH₄Cl. The organic layer was dried over NaSO₄, filter, and concentrated. The residue was purified by flash column chromatography (0% to 10% of MeOH in DCM) to afford N-2 (320 mg, 81% yield). ¹H NMR (600 MHz, Chloroform-d) δ 8.38 (d, J=2.3 Hz, 1H), 8.13 (dd, J=9.2, 2.3 Hz, 1H), 7.66 (d, J=8.8 Hz, 2H), 7.42-7.38 (m, 2H), 7.36-7.26 (m, 4H), 7.09 (d, J=8.6 Hz, 1H), 7.03-6.97 (m, 2H), 6.80 (d, J=9.1 Hz, 2H), 6.63 (d, J=9.4 Hz, 1H), 3.98-3.88 (m, 1H), 3.74-3.62 (m, 4H), 3.42 (s, 4H), 3.29 (t, J=5.2 Hz, 4H), 3.12 (dd, J=13.9, 5.1 Hz, 1H), 3.04 (dd, J=13.9, 7.3 Hz, 1H), 2.85 (s, 2H), 2.58-2.27 (m, 17H), 2.27-2.20 (m, 2H), 2.18-2.10 (m, 2H), 1.95 (d, J=17.3 Hz, 1H), 1.75-1.66 (m, 1H), 1.66-1.59 (m, 1H), 1.47 (s, 9H), 0.96 (s, 3H) ppm. LC/MS (ESI) m/z 1158.4; [M+H]⁺ calcd for C₅₆H₇₂ClF₃N₇O₈S₃⁺: 1158.42.

Preparation of 4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (Intermediate N): HCl dioxane solution (4N, 5 mL) was added to a stirring solution of N-2 (320 mg, 0.28 mmol) in DCM (5 mL). After stirring at room temperature for 2 hours, the reaction mixture was concentrated. The residue was triturated with diethyl ether and dried under vacuum to afford Intermediate L (305 mg, quantitative yield) as an HCl salt, which was used directly in the next step. LC/MS (ESI) m/z 1058.4; [M+H]⁺ calcd for C₅₁H₆₄ClF₃N₇O₆S₃⁺: 1058.37.

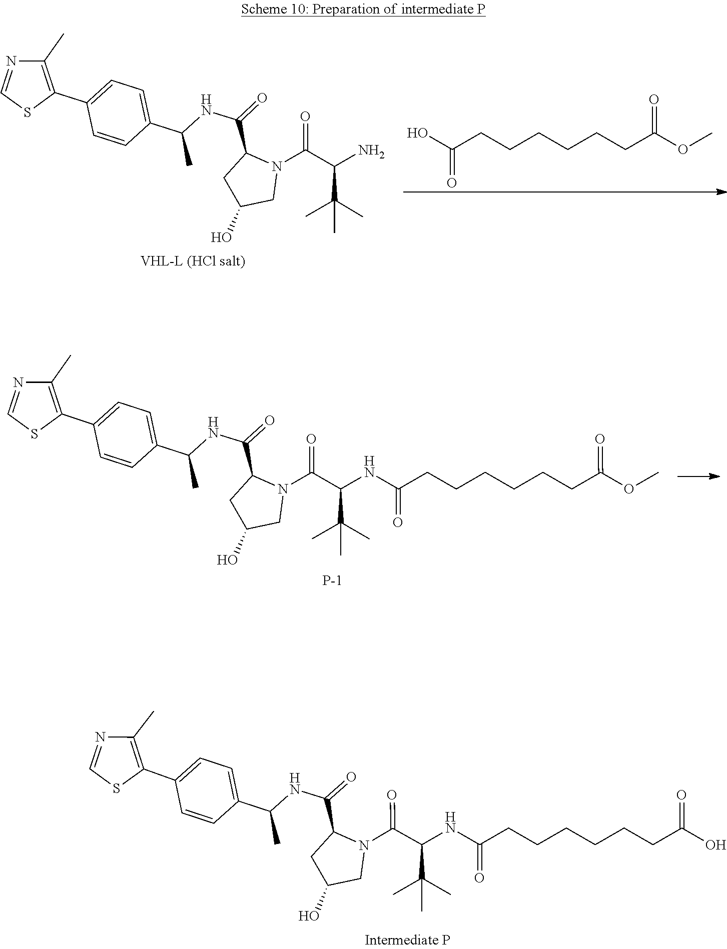

Preparation of methyl 8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoate (P-1): A solution of VHL ligand (VHL-L, 1.0 g, 2.08 mmol, HCl salt) and TEA (1.45 mL, 10.4 mmol) in DCM (10 mL) was added to a solution of 8-methoxy-8-oxooctanoic acid (469 mg, 2.49 mmol) and HATU (1.2 g, 3.12 mmol) in DCM (10 mL). The resulting mixture was stirred at room temperature for 2 hours, then washed with water and sat. aq. NH₄Cl. The organic layers were dried over Na₂SO₄, filtered, and concentrated. The residue was purified by flash column chromatography (0% to 10% of MeOH in DCM) to afford P-1 (820 mg, 64% yield). ¹H NMR (600 MHz, CDCl₃) δ 8.69 (s, 1H), 7.47 (d, J=7.9 Hz, 1H), 7.44-7.41 (m, 2H), 7.41-7.37 (m, 2H), 6.19 (d, J=8.7 Hz, 1H), 5.10 (p, J=7.1 Hz, 1H), 4.74 (t, J=7.9 Hz, 1H), 4.58 (d, J=8.8 Hz, 1H), 4.55-4.50 (m, 1H), 4.12 (d, J=2.0 Hz, 1H), 3.67 (s, 3H), 3.62 (dd, J=11.3, 3.8 Hz, 1H), 2.57-2.49 (m, 5H), 2.31 (t, J=7.5 Hz, 2H), 2.21 (qd, J=8.1, 6.1 Hz, 2H), 2.12-2.04 (m, 1H), 1.67-1.58 (m, 4H), 1.50 (d, J=7.0 Hz, 3H), 1.37-1.28 (m, 4H), 1.06 (s, 9H) ppm. LC/MS (ESI) m/z 615.3; [M+H]⁺ calcd for C₃₂H₄₇N₄O₆S⁺: 615.32.

Preparation of 8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoic acid (Intermediate P): To a solution of P-1 (820 mg, 1.33 mmol) in MeOH/THF (5 mL/5 mL) was added a solution of LiOH·H₂O (168 mg, 4.0 mmol) in water (2 mL). The resulting mixture was stirred at room temperature for 3 hours. The reaction mixture was concentrated to remove the organic solvents, then adjusted to pH=3-4 with 1N aq. HCl. The precipitate was collected via filtration and washed with water, then dried under vacuum to afford Intermediate P (730 mg, 91% yield). ¹H NMR (600 MHz, DMSO) δ 8.99 (s, 1H), 8.38 (d, J=7.8 Hz, 1H), 7.79 (d, J=9.3 Hz, 1H), 7.46-7.41 (m, 2H), 7.41-7.36 (m, 2H), 4.92 (p, J=7.1 Hz, 1H), 4.52 (d, J=9.3 Hz, 1H), 4.43 (t, J=8.0 Hz, 1H), 4.30-4.26 (m, 1H), 3.65-3.57 (m, 2H), 2.46 (s, 3H), 2.27-2.21 (m, 1H), 2.18 (t, J=7.4 Hz, 2H), 2.14-2.07 (m, 1H), 2.05-1.98 (m, 1H), 1.83-1.76 (m, 1H), 1.56-1.41 (m, 4H), 1.38 (d, J=7.0 Hz, 3H), 1.30-1.19 (m, 4H), 0.94 (s, 9H) ppm. LC/MS (ESI) m/z 601.3; [M+H]⁺ calcd for C₃₁H₄₅N₄O₆S⁺: 601.31

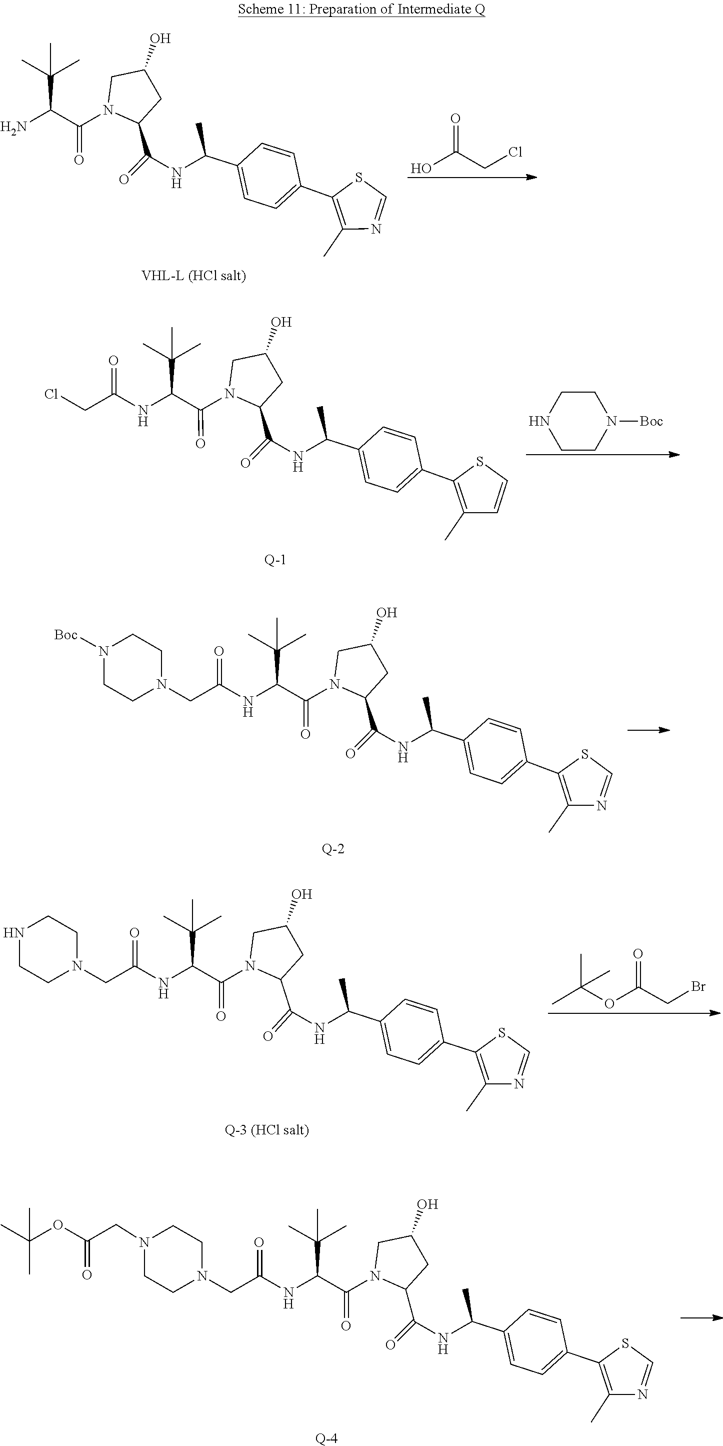

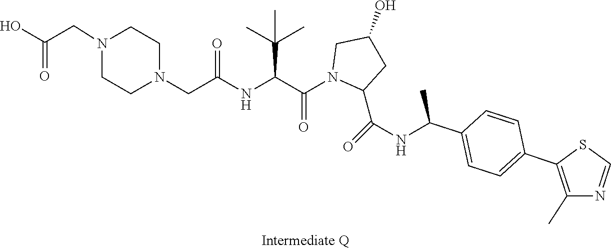 [SMILES: Cc1ncsc1-c1ccc([C@H](C)NC(=O)C2C[C@@H](O)CN2C(=O)[C@@H](NC(=O)CN2CCN(CC(=O)O)CC2)C(C)(C)C)cc1 ; "Intermediate Q"]

Preparation of (2S,4R)-1-((S)-2-(2-chloroacetamido)-3,3-dimethylbutanoyl)-4-hydroxy-N—((S)-1-(4-(4-methylthiazol-5-yl)phenyl)ethyl)pyrrolidine-2-carboxamide (Q-1): A solution of 2-chloroacetic acid (294 mg, 3.12 mmol), HATU (1.58 g, 4.16 mmol), VHL-L (1 g, 2.08 mmol, HCl salt) and TEA (1.74 mL, 12.47 mmol) in DCM (40 mL) was stirred at room temperature for hours. The reaction mixture was washed with water and sat. aq. NH₄Cl. The organic layers were dried over Na₂SO₄, filtered, and concentrated. The residue was purified by flash column chromatography (0% to 10% of MeOH in DCM) to afford Q-1 (870 mg, 81% yield). ¹H NMR (600 MHz, CDCl₃) δ 8.67 (s, 1H), 7.43-7.39 (m, 2H), 7.38-7.33 (m, 3H), 7.23 (d, J=8.6 Hz, 1H), 5.08 (p, J=7.1 Hz, 1H), 4.73 (t, J=7.7 Hz, 1H), 4.57-4.52 (m, 2H), 4.10-4.03 (m, 2H), 4.00 (d, J=2.0 Hz, 1H), 3.64 (dd, J=11.3, 4.0 Hz, 1H), 2.76-2.71 (m, 1H), 2.59-2.51 (m, 4H), 2.09-2.02 (m, 1H), 1.49 (d, J=7.0 Hz, 3H), 1.07 (s, 9H) ppm. LC/MS (ESI) m/z 521.2; [M+H]⁺ calcd for C₂₅H₃₄ClN₄O₄S⁺: 521.20.

Preparation of tert-butyl 4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazine-1-carboxylate (Q-2): A solution of Q-1 (380 mg, 0.73 mmol), DIPEA (507 μL, 2.92 mmol) and tert-butyl piperazine-1-carboxylate (203 mg, 1.09 mmol) in DCM (8 mL) was stirred at room temperature for 3 days. The reaction mixture was washed with sat. aq. NH₄Cl. The organic layers were dried over Na₂SO₄, filtered, and concentrated. The residue was purified by flash column chromatography (0% to 10% of MeOH in DCM) to afford Q-2 (500 mg, quantitative yield). ¹H NMR (600 MHz, CDCl₃) δ 8.69 (s, 1H), 7.84 (d, J=8.2 Hz, 1H), 7.47 (d, J=7.8 Hz, 1H), 7.44-7.40 (m, 2H), 7.40-7.36 (m, 2H), 5.10 (p, J=7.1 Hz, 1H), 4.78 (t, J=7.8 Hz, 1H), 4.53 (s, 1H), 4.45 (d, J=8.2 Hz, 1H), 4.21-4.16 (m, 1H), 3.61 (dd, J=11.4, 3.7 Hz, 1H), 3.55-3.42 (m, 4H), 3.14-3.01 (m, 3H), 2.62-2.56 (m, 1H), 2.55 (s, 3H), 2.54-2.48 (m, 4H), 2.12-2.05 (m, 1H), 1.53-1.46 (m, 12H), 1.09 (s, 9H) ppm. LC/MS (ESI) m/z 671.3; [M+H]⁺ calcd for C₃₄H₅₁N₆O₆S⁺: 671.36.

Preparation of (2S,4R)-1-((S)-3,3-dimethyl-2-(2-(piperazin-1-yl)acetamido)butanoyl)-4-hydroxy-N—((S)-1-(4-(4-methylthiazol-5-yl)phenyl)ethyl)pyrrolidine-2-carboxamide hydrochloride (Q-3): HCl dioxane solution (4N, 5 mL) was added to a solution of Q-2 (500 mg, 0.75 mmol) in DCM (5 mL). After stirring at room temperature for 2 hours, the reaction mixture was concentrated. The residue was triturated with diethyl ether and dried under vacuum to afford Q-3 (570 mg, quantitative yield) as an HCl salt, which was used directly in the next step. LC/MS (ESI) m/z 571.3; [M+H]⁺ calcd for C₂₉H₄₃N₆O₄S⁺: 571.31.

Preparation of tert-butyl 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetate (Q-4): To a solution of Q-3 (570 mg, 0.94 mmol) and DIPEA (1.31 mL, 7.51 mmol) in DCM (20 mL) was added tert-butyl 2-bromoacetate (549 mg, 2.82 mmol). The resulting mixture was stirred at room temperature overnight. The reaction mixture was washed with sat. aq. NH₄Cl. The organic layer was dried over Na₂SO₄, filtered, and concentrated. The residue was purified by flash column chromatography (0% to 10% of MeOH in DCM) to afford Q-4 (390 mg, 78% yield in 3 steps). ¹H NMR (600 MHz, CDCl₃) δ 8.70 (s, 1H), 7.88 (d, J=8.2 Hz, 1H), 7.48 (d, J=7.8 Hz, 1H), 7.45-7.41 (m, 2H), 7.41-7.37 (m, 2H), 5.10 (p, J=7.1 Hz, 1H), 4.79 (t, J=7.8 Hz, 1H), 4.53 (s, 1H), 4.45 (d, J=8.2 Hz, 1H), 4.23 (d, J 24=11.4 Hz, 1H), 3.60 (dd, J=11.4, 3.7 Hz, 1H), 3.20-2.99 (m, 5H), 2.79-2.57 (m, 9H), 2.55 (s, 3H), 2.13-2.05 (m, 1H), 1.51-1.48 (m, 12H), 1.09 (s, 9H) ppm. LC/MS (ESI) m/z 685.4; [M+H]⁺ calcd for C₃₅H₅₃N₆O₆S⁺: 685.37.

Preparation of 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetic acid (Intermediate Q): HCl dioxane solution (4N, 10 mL) was added to a solution of Q-4 (390 mg, 0.57 mmol) in DCM (10 mL). The resulting mixture was stirred at room temperature overnight. The reaction mixture was concentrated and triturated with diethyl ether, collected the precipitate via filtration, and dried under vacuum to afford Intermediate Q (352 mg, 98% yield), which was used directly in the next step. LC/MS (ESI) m/z 629.3; [M+H]⁺ calcd for C₃₁H₄₅N₆O₆S⁺: 629.31.

### Preparation of Compounds in Table 1

### General Procedure A

Step 1: A mixture of a halide ester (1.0 equiv.), 1-Boc-piperazine (1.2 equiv.), and DIPEA (3.0 equiv.) or K₂CO₃ (1.5 equiv.) in DMF or DMSO was heated at 50° C.-120° C. overnight. The resulting mixture was cooled to room temperature and diluted with water, then extracted with EtOAc. The combined organic layers were washed with water and sat. aq. NH₄Cl, dried over Na₂SO₄, filtered, and concentrated. The residue was purified by flash column chromatography to afford the corresponding ester intermediate.

Step 2: To a solution of the ester intermediate from Step 1 (1.0 equiv.) in MeOH/THF was added a solution of LiOH·OH (5 equiv.) in water, then stirred at 40° C. overnight. The reaction mixture was concentrated to remove the organic solvents, then adjusted to pH=5-6 with 10% aq. citric acid and extracted with EtOAc. The combined organic layers were washed with brine, dried over Na₂SO₄, filtered, and concentrated to afford the corresponding carboxylic acid intermediate.

Step 3: A solution of an amine (1.0 equiv.) and DIPEA (5.0 equiv.) in DMF was added to a solution of carboxylic acid intermediate from step 2 (1.0 equiv.) and HATU (1.2 equiv.) in DMF. The resulting mixture was stirred at room temperature for 2 hours, then diluted with EtOAc, and washed with sat. aq. NH₄Cl. The organic layer was dried over Na₂SO₄, filtered, and concentrated. The residue was purified by flash column chromatography to afford the corresponding amide intermediate.

Step 4: To a solution of the amide intermediate from step 3 in DCM was added HCl dioxane solution (4N). The resulting mixture was stirred at room temperature for 1-2 hours. The reaction mixture was concentrated to afford the crude piperazine intermediate as an HCl salt, which was used directly in the next step.

Step 5: A suspension of aldehyde (1.0 equiv.), piperazine intermediate from step 4 (1.2-1.6 equiv.), TEA (20.0 equiv.) and NaBH(OAc)₃ (2.0 equiv.) in DCE was stirred at room temperature overnight. The reaction mixture was washed with sat. aq. NH₄Cl, dried over Na₂SO₄, filtered, and concentrated. The residue was purified by preparative TLC (DCM/MeOH) to afford the corresponding final product.

### General Procedure B

To a solution of azide (1.0 equiv.) and alkyne (1.0 equiv.) in THF/t-BuOH/H₂O were added Vc-Na (0.05 equiv.) and CuSO₄ (0.05 equiv.), then the resulting mixture was stirred at 50° C. overnight. The reaction mixture was cooled to room temperature and concentrated. The residue was purified by preparative TLC (DCM/MeOH) to afford the desired compounds.

### General Procedure C

Step 1: A mixture of amine (1 equiv.), halide (1.1 equiv.), and base (2 equiv.) in DMF was stirred at room temperature overnight. The reaction mixture was diluted with EtOAc, then washed with water and sat. aq. NH₄Cl, dried over Na₂SO₄, filtered, and concentrated. The residue was purified by flash column chromatography to afford tert-butyl ester intermediate.

Step 2: To a solution of the tert-butyl ester intermediate from step 1 (1.0 equiv.) in DCM was added HCl dioxane solution (4N), then stirred at room temperature overnight. The reaction mixture was concentrated to afford crude carboxylic acid intermediate, which was used directly in the next step.

Step 3: A solution of VHL-L (1 equiv., HCl salt) and TEA (5 equiv.) in DCM was added to a solution of carboxylic acid intermediate from step 2 (1 equiv.), TEA (5 equiv.), and HATU (1.1 equiv.) in DCM. The resulting mixture was stirred at room temperature for 7 hours. The reaction mixture was washed with sat. aq. NH₄Cl, dried over Na₂SO₄, filtered, and concentrated. The residue was purified by flash column chromatography to afford an ethyl ester intermediate.

Step 4: To a solution of the ethyl ester intermediate from step 3 (1.0 equiv.) in MeOH/THF was added a solution of LiOH·H₂O (3.0-5.0 equiv.) in water and the resulting mixture was stirred at room temperature overnight. The reaction mixture was concentrated to remove the organic solvents, then adjusted to pH=5-6 with 10% aq. citric acid. The precipitate was collected via filtration, then washed with water and dried under vacuum to afford a carboxylic acid intermediate.

Step 5: A solution of the carboxylic acid intermediate from step 4 (1 equiv.), HATU (1.1 equiv.), Intermediate N (1 equiv., HCl salt) and TEA (10 equiv.) in DMF was stirred at room temperature overnight. The reaction mixture was diluted with EtOAc and washed with sat. aq. NH₄Cl solution. The organic layer was dried over Na₂SO₄, filtered, and concentrated. The residue was purified by preparative TLC (DCM/MeOH) to afford the desired compound.

### General Procedure D

Step 1: A suspension of G-2 (1.0 equiv.), amine (1.2 equiv.) and NaBH(OAc)₃ (2.0 equiv.) in DCM was stirred at room temperature for 3 hours. The reaction mixture was washed with sat. aq. NH₄Cl, dried over Na₂SO₄, filtered, and concentrated. The residue was purified by flash column chromatography to afford a Boc-amine intermediate.

Step 2: To a solution of the Boc-amine intermediate from step 1 (1.0 equiv.) in DCM was added HCl dioxane solution (4N), then stirred at room temperature for 1-2 hours. The reaction mixture was concentrated to afford the corresponding amine intermediate as an HCl salt, which was used directly in the next step.

Step 3: A solution of the amine intermediate from step 2 (1.0 equiv., HCl salt), DIPEA (8.0 equiv.) and 4-fluoro-3-((trifluoromethyl)sulfonyl)benzenesulfonamide or 4-fluoro-3-nitrobenzenesulfonamide (1.0 equiv.) in DMSO was stirred at room temperature overnight. The resulting mixture was diluted with EtOAc and washed with water and sat. aq. NH₄Cl. The organic layers were dried over Na₂SO₄, filtered, and concentrated. The residue was purified by flash column chromatography to afford a sulfonamide intermediate.

Step 4: To a solution of Intermediate A or B (1.0 equiv.), DMAP (5.0 equiv.), and EDC (5.0 equiv.) in DCM was added the sulfonamide intermediate from step 3 (1.0 equiv.). The resulting mixture was stirred at room temperature overnight. The reaction mixture was washed with water and sat. aq. NH₄Cl, dried over Na₂SO₄, filtered, and concentrated. The residue was purified by flash column chromatography to afford a Boc-amine-intermediate.

Step 5: To a solution of the Boc-amine-intermediate from step 4 (1.0 equiv.) in DCM was added HCl dioxane solution (4N), then stirred at room temperature for 1−2 hours. The reaction mixture was concentrated to afford an amine HCl salt intermediate, which was used directly in the next step.

Step 6: A solution of the amine HCl salt from step 5 (1.0 equiv.) and TEA (8.0 equiv.) in DCM (or DMF) was added to a solution of Intermediate P (1.2 equiv.) and HATU (1.5 equiv.) in DCM (or DMF). The resulting mixture was stirred at room temperature for 2 hours, then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compound.

### General Procedure E

Step 1: To a solution of carboxylic acid (1.0 equiv.), DMAP (5.0 equiv.), and EDC (5.0 equiv.) in DCM was added sulfonamide (1.0 equiv.). The resulting mixture was stirred at room temperature overnight. The reaction mixture was washed with water and sat. aq. NH₄Cl, dried over Na₂SO₄, filtered, and concentrated. The residue was purified by flash column chromatography to afford the corresponding N-acyl-sulfonamide intermediate with a Boc-protecting group.

Step 2: To a solution of the intermediate from step 1 (1.0 equiv.) in DCM was added HCl dioxane solution (4N), then stirred at room temperature for 1−2 hours. The reaction mixture was concentrated to afford the corresponding amine-HCl salt intermediate, which was used directly in the next step.

Step 3: A solution of the amine-HCl salt intermediate from step 2 (1.0 equiv.) and TEA (8.0 equiv.) in DCM (or DMF) was added to a solution of carboxylic acid (1.2 equiv.) and HATU (1.5 equiv.) in DCM (or DMF). The resulting mixture was stirred at room temperature for 2 hours. then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compound.

### General Procedure F

Step 1: A suspension of aldehyde (1.0 equiv.), amine (1.2-1.5 equiv.), TEA (3.0-5.0 equiv.) and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by flash column chromatography to afford an ethyl ester intermediate.

Step 2: To a solution of the ethyl ester intermediate from step 1 (1.0 equiv.) in MeOH/THF was added a solution of LiOH·H₂O (3.0−5.0 equiv.) in water, then stirred at room temperature overnight. The reaction mixture was concentrated to remove the organic solvents, then adjusted to pH=5-6 with 10% aq. citric acid. The precipitate was collected via filtration, then washed with water and dried under vacuum to afford the corresponding carboxylic acid intermediate.

Step 3: To a solution of the carboxylic acid intermediate from step 2 (1.0 equiv.), DMAP (5.0 equiv.) and EDC (5.0 equiv.) in DCM was added sulfonamide (1.0 equiv.). The resulting mixture was stirred at room temperature overnight. The reaction mixture was washed with water and sat. aq. NH₄Cl, dried over Na₂SO₄, filtered, and concentrated. The residue was purified by flash column chromatography to afford a Boc-protected intermediate.

Step 4: To a solution of the intermediate from step 3 (1.0 equiv.) in DCM was added HCl dioxane solution (4N), then stirred at room temperature for 1−2 hours. The reaction mixture was concentrated to afford crude amine-HCl salt, which was used directly in the next step.

Step 5: A solution of the amine-HCl salt from step 4 (1.0 equiv.) and TEA (8.0 equiv.) in DCM (or DMF) was added to a solution of carboxylic acid (1.2 equiv.) and HATU (1.5 equiv.) in DCM (or DMF). The resulting mixture was stirred at room temperature for 2 hours, then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compound.

### General Procedure G

Step 1: A suspension of aldehyde (1.0 equiv.), amine (1.2-1.5 equiv.), TEA (3.0-5.0 equiv.), and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by flash column chromatography to afford the corresponding reductive amination product with a Boc-protecting group.

Step 2: To a solution of the product from step 1 (1.0 equiv.) in DCM was added HCl dioxane solution (4N), then stirred at room temperature for 2 hours. The reaction mixture was concentrated to afford the corresponding amine-HCl salt intermediate, which was used directly in the next step.

Step 3: A solution of the crude amine-HCl salt from step 2 (1.0 equiv.) and TEA (8.0 equiv.) in DCM (or DMF) was added to a solution of Intermediate P (1.2 equiv.), and HATU (1.5 equiv.) in DCM (or DMF). The resulting mixture was stirred at room temperature for 2 hours, then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compound.

### General Procedure H

A solution of amine (1.0 equiv., HCl salt) and TEA (8.0 equiv.) in DCM (or DMF) was added to a solution of acid (1.2 equiv.) and HATU (1.5 equiv.) in DCM (or DMF). The resulting mixture was stirred at room temperature for 2 hours, then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compounds.

### General Procedure J

Step 1: A solution of amine hydrochloride (1.0 equiv.), DIPEA (8.0 equiv.), and 4-fluoro-3-((trifluoromethyl)sulfonyl)benzenesulfonamide or 4-fluoro-3-nitrobenzenesulfonamide (1.0 equiv.) in DMSO was stirred at room temperature overnight. The reaction mixture was diluted with EtOAc and washed with sat. aq. NH₄Cl. The organic layer was dried over Na₂SO₄, filtered, and concentrated. The residue was purified by flash column chromatography to afford a sulfonamide intermediate.

Step 2: To a solution of Intermediate A or B (1.0 equiv.), DMAP (5.0 equiv.), and EDC (5.0 equiv.) in DCM was added the sulfonamide intermediate from step 1 (1.0 equiv.). The resulting mixture was stirred at room temperature overnight, then washed with water and sat. aq. NH₄Cl. The organic layers were dried over Na₂SO₄, filtered, and concentrated. The residue was purified by flash column chromatography to afford the corresponding N-acyl-sulfonamide with a Boc-protecting group.

Step 3: To a solution of the product from step 2 (1.0 equiv.) in DCM was added HCl dioxane solution (4N), then stirred at room temperature for 1−2 hours. The reaction mixture was concentrated to afford crude amine-HCl salt intermediate, which was used directly in the next step.

Step 4: A solution of the amine-HCl salt from step 3 (1.0 equiv.) and TEA (8.0 equiv.) in DCM (or DMF) was added to a solution of intermediate P (1.2 equiv.), and HATU (1.5 equiv.) in DCM (or DMF). The resulting mixture was stirred at room temperature for 2 h, then washed with sat. aq. NH₄Cl (or diluted with EtOAc and washed with sat. aq. NHCl₄). The organic layer was dried over Na₂SO₄, filtered, and concentrated. The residue was purified by preparative TLC (DCM/MeOH) to afford the desired compound.

### General Procedure K

A mixture of amine (2.0 equiv.), TEA (8.0 equiv.), aldehyde (1.0 equiv.), and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by preparative TLC (DCM/MeOH) to afford the desired compound.

### Example 1: Preparation of Compounds #1-3

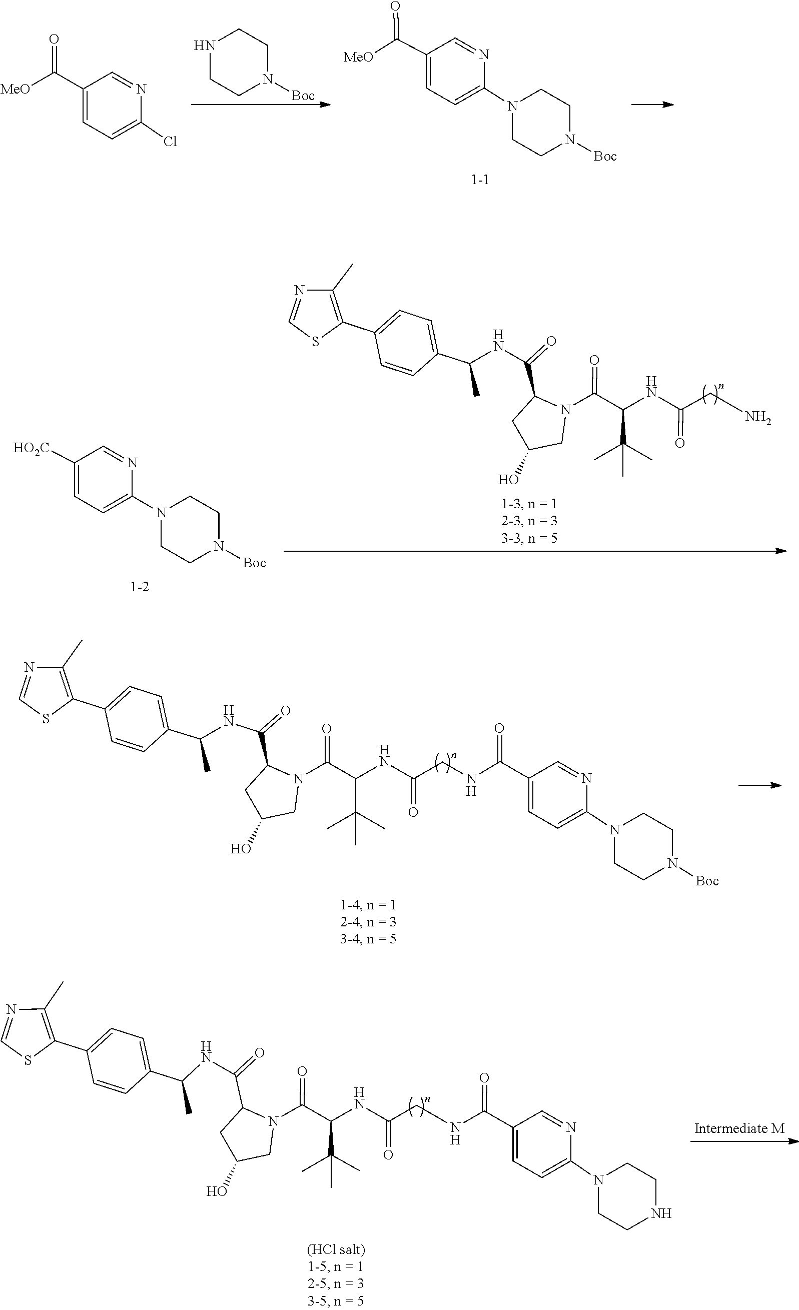

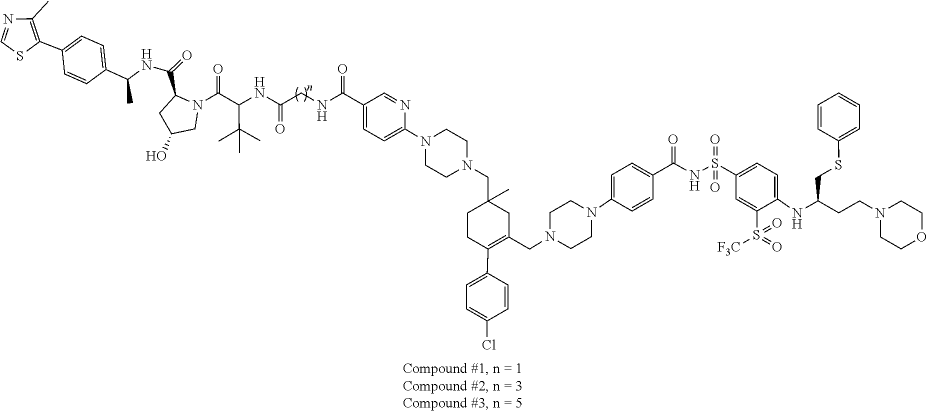

Compounds #1-3 were prepared by following General Procedure A. tert-butyl 4-(5-(methoxycarbonyl)pyridin-2-yl)piperazine-1-carboxylate (1-1) (890 mg, 95% yield). ¹H NMR (600 MHz, CDCl₃) δ 8.82 (dd, J=2.3, 0.7 Hz, 1H), 8.06 (dd, J=9.0, 2.4 Hz, 1H), 6.60 (dd, J=9.0, 0.7 Hz, 1H), 3.90 (s, 3H), 3.74-3.67 (m, 4H), 3.57 (t, J=5.2 Hz, 4H), 1.51 (s, 9H) ppm. LC/MS (ESI) m/z 322.3; [M+H]⁺ calcd for C₁₆H₂₄N₃O₄⁺: 322.18.

6-(4-(tert-butoxycarbonyl)piperazin-1-yl)nicotinic acid (1-2) (266 mg, 93% yield). ¹H NMR (600 MHz, DMSO) δ 8.64 (d, J=2.4 Hz, 1H), 7.95 (dd, J=9.1, 2.4 Hz, 1H), 6.86 (d, J=9.1 Hz, 1H), 3.69-3.60 (m, 4H), 3.43 (t, J=5.3 Hz, 4H), 1.43 (s, 9H) ppm. LC/MS (ESI) m/z 308.2; [M+H]⁺ calcd for C₁₅H₂₂N₃O₄⁺: 308.16.

tert-butyl 4-(5-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)pyridin-2-yl)piperazine-1-carboxylate (1-4) (18 mg, 57% yield). ¹H NMR (600 MHz, Chloroform-d) δ 8.72 (d, J=2.5 Hz, 1H), 8.67 (s, 1H), 8.09 (d, J=7.8 Hz, 1H), 7.98 (dd, J=9.0, 2.5 Hz, 1H), 7.90 (d, J=9.2 Hz, 1H), 7.75 (t, J=4.8 Hz, 1H), 7.36 (s, 4H), 6.57 (d, J=9.1 Hz, 1H), 5.10 (p, J=7.1 Hz, 1H), 4.79 (t, J=8.4 Hz, 1H), 4.69 (d, J=9.3 Hz, 1H), 4.46-4.35 (m, 2H), 4.06 (dd, J=17.2, 4.1 Hz, 1H), 3.84 (d, J=11.2 Hz, 1H), 3.71-3.61 (m, 5H), 3.58 (dd, J=11.4, 3.1 Hz, 1H), 3.53 (dd, J=6.7, 4.0 Hz, 4H), 2.51 (s, 3H), 2.24 (dd, J=13.6, 7.7 Hz, 1H), 2.16 (ddd, J=13.3, 9.2, 4.3 Hz, 1H), 1.52 (d, 3H), 1.49 (s, 9H), 1.04 (s, 9H). LC/MS (ESI) m/z 791.4; [M+H]⁺ calcd for C₄₀H₅₅N₈O₇S⁺: 791.39

tert-butyl 4-(5-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)carbamoyl)pyridin-2-yl)piperazine-1-carboxylate (2-4): (17 mg, 55% yield). LC/MS (ESI) m/z 819.3; [M+H]⁺ calcd for C₄₂H₅₉N₈O₇S⁺: 819.42. tert-butyl 4-(5-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)pyridin-2-yl)piperazine-1-carboxylate (3-4): (19 mg, 61% yield). ¹H NMR (600 MHz, Chloroform-d) δ 8.68 (s, 1H), 8.60 (d, J=2.4 Hz, 1H), 7.96 (dd, J=9.0, 2.5 Hz, 1H), 7.51 (d, J=7.8 Hz, 1H), 7.40 (d, J=8.3 Hz, 2H), 7.36 (d, J=8.2 Hz, 2H), 6.78 (t, J=5.8 Hz, 1H), 6.61 (d, J=8.9 Hz, 1H), 6.53 (d, J=9.0 Hz, 1H), 5.08 (p, J=7.1 Hz, 1H), 4.67 (t, J=8.1 Hz, 1H), 4.60 (d, J=9.1 Hz, 1H), 4.48 (s, 1H), 4.05 (d, J=11.3 Hz, 1H), 3.60 (dd, J=6.7, 3.9 Hz, 5H), 3.52 (dd, J=6.7, 4.0 Hz, 4H), 3.40 (qd, J=6.8, 3.6 Hz, 2H), 2.52 (s, 3H), 2.42-2.35 (m, 1H), 2.31-2.18 (m, 3H), 2.13-2.06 (m, 1H), 1.65 (dq, J=14.1, 7.3 Hz, 2H), 1.58 (p, J 26=7.0 Hz, 2H), 1.51-1.45 (m, 12H), 1.37 (p, J=7.7 Hz, 2H), 1.03 (s, 9H) ppm. LC/MS (ESI) m/z 847.4; [M+H]⁺ calcd for C₄₄H₆₃N₈O₇S⁺: 847.45. N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-6-(piperazin-1-yl)nicotinamide hydrochloride (1-5): (16 mg, quantitative yield). LC/MS (ESI) m/z 691.2; [M+H]⁺ calcd for C₃₅H₄₇N₈O₅S⁺: 691.34. N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-6-(piperazin-1-yl)nicotinamide hydrochloride (2−5): (18 mg, quantitative yield). LC/MS (ESI) m/z 719.2; [M+H]⁺ calcd for C₃₇H₅₁N₈O₅S⁺: 719.37.

N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-6-(piperazin-1-yl)nicotinamide hydrochloride (3-5): (17 mg, quantitative yield). LC/MS (ESI) m/z 747.6; [M+H]⁺ calcd for C₃₉H₅₅N₈O₅S⁺: 747.40.

6-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)nicotinamide (compound #1) (7.3 mg, 22% yield). ¹H NMR (600 MHz, Chloroform-d) δ 8.67 (s, 1H), 8.62 (d, J=3.7 Hz, 1H), 8.33 (d, J=2.3 Hz, 1H), 8.08 (d, J=9.1 Hz, 1H), 7.86 (d, J=8.9 Hz, 1H), 7.75 (d, J=8.4 Hz, 2H), 7.65 (d, J=33.6 Hz, 1H), 7.39-7.32 (m, 6H), 7.32-7.26 (m, 4H), 7.24 (t, J=7.3 Hz, 1H), 7.04-6.96 (m, 2H), 6.75 (d, 2H), 6.59 (d, J=9.4 Hz, 1H), 6.46 (s, 1H), 5.08 (p, J=6.9 Hz, 1H), 4.75 (t, J=8.3 Hz, 1H), 4.61 (s, 1H), 4.46 (s, 1H), 4.20 (d, J=16.6 Hz, 1H), 4.14-4.07 (m, 1H), 3.98 (d, J=11.2 Hz, 1H), 3.87 (s, 1H), 3.70-3.50 (m, 10H), 3.23 (s, 4H), 3.10 (dd, J=13.8, 4.9 Hz, 1H), 3.01 (dd, J=13.9, 7.1 Hz, 1H), 2.87 (q, J=12.6, 12.2 Hz, 2H), 2.72-2.57 (m, 4H), 2.51 (s, 3H), 2.47-2.16 (m, 21H), 2.10 (t, J=7.4 Hz, 1H), 1.90 (d, J=17.3 Hz, 1H), 1.71-1.58 (m, 2H), 1.52-1.40 (m, 4H), 1.05 (s, 9H), 1.00 (s, 3H) ppm. LC/MS (ESI) m/z 1662.5; [M+H]⁺ calcd for C₈₂H₁₀₀ClF₃N₁₃O₁₁S₄⁺: 1662.62.

6-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)nicotinamide (compound #2) (4.7 mg, 20% yield). ¹H NMR (600 MHz, Chloroform-d) δ 8.68 (s, 1H), 8.57-8.51 (m, 1H), 8.36-8.30 (m, 1H), 8.09 (d, J=9.2 Hz, 1H), 7.93 (t, J=9.1 Hz, 1H), 7.73 (s, 2H), 7.46-7.33 (m, 6H), 7.28 (s, 4H), 7.03-6.96 (m, 2H), 6.87 (s, 1H), 6.74 (d, J=8.6 Hz, 2H), 6.59 (d, J=9.2 Hz, 1H), 6.51 (s, 1H), 5.09 (p, J=6.9 Hz, 1H), 4.64 (dt, J=37.1, 8.2 Hz, 1H), 4.55-4.45 (m, 2H), 4.09 (d, J=11.0 Hz, 1H), 3.88 (s, 1H), 3.73-3.33 (m, 12H), 3.20 (s, 4H), 3.10 (dd, J=14.1, 4.9 Hz, 1H), 3.05-2.97 (m, 1H), 2.84 (dd, 2H), 2.73-2.60 (m, 4H), 2.51 (d, J=1.2 Hz, 3H), 2.47-2.19 (m, 20H), 2.10 (s, 2H), 1.92 (s, 4H), 1.71-1.58 (m, 2H), 1.51-1.43 (m, 4H), 1.05 (s, 9H), 0.99 (d, J=4.6 Hz, 3H) ppm. LC/MS (ESI) m/z 1690.8; [M+H]⁺ calcd for C₈₄H₁₀₄ClF₃N₁₃O₁₁S₄⁺: 1690.65.

6-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)nicotinamide (compound #3) (5.6 mg, 20% yield). ¹H NMR (600 MHz, Chloroform-d) δ 8.68 (s, 1H), 8.59 (d, J=2.1 Hz, 1H), 8.33 (d, J=2.2 Hz, 1H), 8.09 (d, J=9.2 Hz, 1H), 7.93 (dd, J=8.8, 2.4 Hz, 1H), 7.75 (s, 2H), 7.47 (dd, J=41.3, 7.8 Hz, 1H), 7.40−7.32 (m, 6H), 7.32-7.21 (m, 5H), 7.02-6.95 (m, 2H), 6.89-6.82 (m, 1H), 6.75 (d, J=8.8 Hz, 2H), 6.56 (dd, J=31.0, 16.4 Hz, 3H), 5.07 (td, J=7.2, 3.4 Hz, 1H), 4.64 (dt, J=20.8, 8.2 Hz, 1H), 4.57 (dd, J=9.1, 4.1 Hz, 1H), 4.47 (s, 1H), 4.05 (dd, J=11.3, 6.2 Hz, 1H), 3.87 (s, 1H), 3.70-3.60 (m, 4H), 3.60-3.49 (m, 5H), 3.46-3.37 (m, 2H), 3.21 (s, 4H), 3.14-3.06 (m, 1H), 3.01 (dd, J=13.8, 7.2 Hz, 1H), 2.85 (p, J=12.5, 12.0 Hz, 2H), 2.70-2.57 (m, 4H), 2.51 (s, 3H), 2.46-2.19 (m, 20H), 2.10 (dt, J=13.7, 7.4 Hz, 2H), 1.88 (d, J=17.3 18 Hz, 1H), 1.73-1.53 (m, 4H), 1.46 (t, J=6.9 Hz, 3H), 1.39-1.31 (m, 1H), 1.03 (s, 9H), 0.98 (s, 3H) ppm. LC/MS (ESI) m/z 1718.5; [M+H]⁺ calcd for C₈₆H₁₀₈ClF₃N₁₃O₁₁S₄⁺: 1718.68.

### Example 2: Preparation of Compounds #4-6

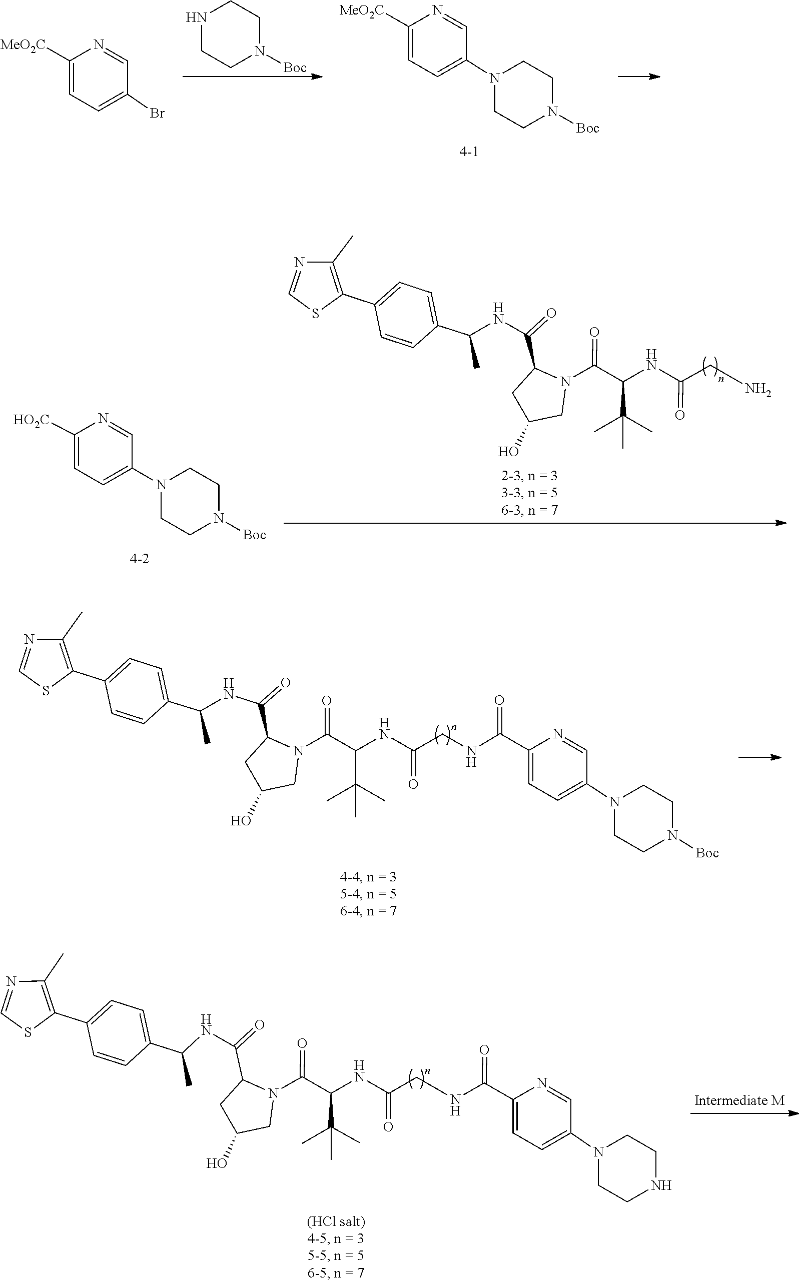

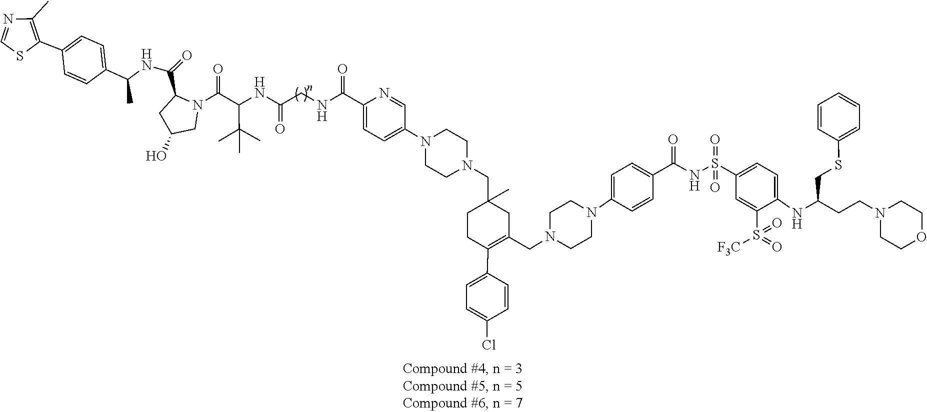

Preparation of tert-butyl 4-(6-(methoxycarbonyl)pyridin-3-yl)piperazine-1-carboxylate (4-1): Under an argon atmosphere, a suspension of methyl 5-bromopicolinate (1.0 equiv.), 1-Boc-piperazine (1.2 equiv.), Cs₂CO₃ (2.5 equiv.), RuPhos (0.1 equiv.) and Pd₂(dba)₃ (0.05 equiv.) in toluene was heated at 80° C. overnight. The reaction mixture was cooled to room temperature and diluted with EtOAc, then washed with water and sat. aq. NH₄Cl. The combined organic layers were dried over Na₂SO₄, filtered, and concentrated. The residue was purified by flash column chromatography to afford 4−1 (580 mg, 78% yield). ¹H NMR (600 MHz, CDCl₃) δ 8.36 (d, J=2.9 Hz, 1H), 8.04 (d, J=8.8 Hz, 1H), 7.18 (dd, J=8.8, 3.0 Hz, 1H), 3.99 (s, 3H), 3.63 (t, 4H), 3.37 (t, 4H), 1.51 (s, 9H) ppm. LC/MS (ESI) m/z 322.2; [M+H]⁺ calcd for C₁₆H₂₄N₃O₄⁺: 322.18.

Compounds #4-6 were prepared from 4-1 by following steps 2-5 of General Procedure A. 5-(4-(tert-butoxycarbonyl)piperazin-1-yl)picolinic acid (4-2) (266 mg, 93% yield). LC/MS (ESI) m/z 308.1; [M+H]⁺ calcd for C₁₅H₂₂N₃O₄⁺: 308.16.

tert-butyl 4-(6-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)carbamoyl)pyridin-3-yl)piperazine-1-carboxylate (4-4) (54 mg, 67% yield). ¹H NMR (600 MHz, CDCl₃) δ 8.69 (s, 1H), 8.17 (d, J=2.9 Hz, 1H), 8.03 (dd, J=8.8, 1.6 Hz, 1H), 7.99 (t, J=6.6 Hz, 1H), 7.62 (d, J=7.6 Hz, 1H), 7.43-7.37 (m, 4H), 7.21 (dd, J=8.8, 2.9 Hz, 1H), 5.11 (p, J=7.1 Hz, 1H), 4.84 (t, J=8.1 Hz, 1H), 4.54 (d, J=7.9 Hz, 1H), 4.51 (s, 1H), 4.24 (d, J=11.5 Hz, 1H), 3.79-3.68 (m, 1H), 3.66-3.44 (m, 7H), 3.31 (t, J=5.2 Hz, 4H), 2.63-2.56 (m, 1H), 2.54 (s, 3H), 2.36-2.24 (m, 2H), 2.13 (ddt, J=13.6, 8.3, 1.8 Hz, 1H), 1.91 (h, J=7.1 Hz, 2H), 1.82-1.73 (m, 1H), 1.53-1.46 (m, 12H), 1.12 (s, 9H) ppm. LC/MS (ESI) m/z 819.6; [M+H]⁺ calcd for C₄₂H₅₉N₈O₇S⁺: 819.42

tert-butyl 4-(6-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)pyridin-3-yl)piperazine-1-carboxylate (5-4) (59 mg, 71% yield). ¹H NMR (600 MHz, CDCl₃) δ 8.69 (s, 1H), 8.17 (d, J=2.9 Hz, 1H), 8.03 (d, J=8.7 Hz, 1H), 7.86 (t, J=6.1 Hz, 1H), 7.56 (d, J=7.8 Hz, 1H), 7.41 (q, J=8.4 Hz, 4H), 7.22 (dd, J=8.8, 2.9 Hz, 1H), 6.21 (d, J=8.2 Hz, 1H), 5.11 (p, J=7.1 Hz, 1H), 4.74 (t, J=7.9 Hz, 1H), 4.57-4.50 (m, 2H), 4.13 (dd, J=11.6, 2.1 Hz, 1H), 3.66-3.57 (m, 5H), 3.44 (td, J=12.4, 6.8 Hz, 2H), 3.38-3.19 (m, 5H), 2.54 (s, 3H), 2.50 (ddt, J=12.1, 7.7, 3.8 Hz, 1H), 2.27-2.15 (m, 2H), 2.13-2.05 (m, 1H), 1.71-1.57 (m, 4H), 1.51 (d, J=1.0 Hz, 12H), 1.47-1.22 (m, 6H), 1.07 (s, 9H) ppm. LC/MS (ESI) m/z 847.6; [M+H]⁺ calcd for C₄₄H₆₃N₈O₇S⁺: 847.45

tert-butyl 4-(6-((8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamoyl)pyridin-3-yl)piperazine-1-carboxylate (6-4) (51 mg, 60% yield). ¹H NMR (600 MHz, CDCl₃) δ 8.69 (s, 1H), 8.17 (d, J=2.9 Hz, 1H), 8.03 (d, J=8.7 Hz, 1H), 7.86 (t, J=6.1 Hz, 1H), 7.56 (d, J=7.8 Hz, 1H), 7.41 (q, J=8.4 Hz, 4H), 7.22 (dd, J=8.8, 2.9 Hz, 1H), 6.21 (d, J=8.2 Hz, 1H), 5.11 (p, J=7.1 Hz, 1H), 4.74 (t, J=7.9 Hz, 1H), 4.57-4.50 (m, 2H), 4.13 (dd, J=11.6, 2.1 Hz, 1H), 3.66-3.57 (m, 5H), 3.44 (td, J=12.4, 6.8 Hz, 2H), 3.38-3.19 (m, 5H), 2.54 (s, 3H), 2.50 (ddt, J=12.1, 7.7, 3.8 Hz, 1H), 2.27-2.15 (m, 2H), 2.13-2.05 (m, 1H), 1.71-1.57 (m, 4H), 1.51 (d, J=1.0 Hz, 12H), 1.47-1.22 (m, 6H), 1.07 (s, 9H) ppm. LC/MS (ESI) m/z 875.5; [M+H]⁺ calcd for C₄₆H₆₇N₈O₇S⁺: 875.48.

N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-5-(piperazin-1-yl)picolinamide hydrochloride (4-5) (28 mg, quantitative yield). LC/MS (ESI) m/z 719.2; [M+H]⁺ calcd for C₃₇H₅₁N₈O₅S⁺: 719.37.

N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-5-(piperazin-1-yl)picolinamide hydrochloride (5-5) (37 mg, quantitative yield). LC/MS (ESI) m/z 747.3; [M+H]⁺ calcd for C₃₉H₅₅N₈O₅S⁺: 747.40.

N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)-5-(piperazin-1-yl)picolinamide hydrochloride (6-5) (30 mg, quantitative yield). LC/MS (ESI) m/z 775.3; [M+H]⁺ calcd for C₄₁H₅₉N₈O₅S⁺: 775.43

5-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)picolinamide (compound #4) (3.1 mg, 26% yield). ¹H NMR (600 MHz, CDCl₃) δ 8.68 (s, 1H), 8.34 (d, J=2.3 Hz, 1H), 8.17-8.10 (m, 2H), 8.00 (t, J=7.7 Hz, 2H), 7.69 (dd, J=8.9, 1.8 Hz, 2H), 7.49 (d, J=7.8 Hz, 1H), 7.42-7.36 (m, 6H), 7.35-7.24 (m, 5H), 7.18 (d, J=8.9 Hz, 2H), 7.02-6.97 (m, 2H), 6.78 (d, J=8.6 Hz, 2H), 6.63 (d, J=9.3 Hz, 1H), 5.09 (p, J=7.1 Hz, 1H), 4.76 (t, J=8.1 Hz, 1H), 4.57 (d, J=8.4 Hz, 1H), 4.49 (s, 1H), 4.17-4.11 (m, 1H), 3.91 (s, 1H), 3.66 (d, J=8.3 Hz, 4H), 3.61-3.57 (m, 1H), 3.54-3.46 (m, 2H), 3.36-3.22 (m, 8H), 3.12 (dd, J=13.9, 5.1 Hz, 1H), 3.04 (dd, J=13.9, 7.2 Hz, 1H), 2.85 (s, 2H), 2.79-2.68 (m, 4H), 2.56-2.17 (m, 22H), 2.12 (dd, J=13.7, 8.2 Hz, 2H), 1.99-1.87 (m, 3H), 1.74-1.60 (m, 2H), 1.53-1.39 (m, 4H), 1.08 (s, 9H), 0.99 (s, 3H) ppm. LC/MS (ESI) m/z 1690.6; [M+H]⁺ calcd for Cs₄H₁₀₄ClF₃N₁₃O₁₁S₄⁺: 1690.65.

5-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)picolinamide (compound #5) (3.3 mg, 21% yield). ¹H NMR (600 MHz, CDCl₃) δ 8.69 (s, 1H), 8.34 (d, J=2.3 Hz, 1H), 8.13 (dd, J=9.0, 2.5 32 Hz, 2H), 7.97 (d, J=8.8 Hz, 1H), 7.87 (t, J=6.1 Hz, 1H), 7.70 (d, J=8.6 Hz, 2H), 7.46 (dd, J=8.0, 6.1 Hz, 1H), 7.43-7.36 (m, 6H), 7.35-7.23 (m, 5H), 7.18-7.12 (m, 1H), 7.02-6.98 (m, 2H), 6.78 (d, J=8.9 Hz, 2H), 6.62 (d, J=9.3 Hz, 1H), 6.48 (t, J=8.3 Hz, 1H), 5.09 (p, J=7.1 Hz, 1H), 4.73 (t, J=8.1 Hz, 1H), 4.61 (dd, J=9.1, 1.2 Hz, 1H), 4.49 (s, 1H), 4.08 (d, J=11.4 Hz, 1H), 3.95-3.87 (m, 1H), 3.67 (tt, J=12.1, 6.1 Hz, 4H), 3.58 (dd, J=11.4, 3.4 Hz, 1H), 3.47 2-3.41 (m, 2H), 3.28 (dt, J=18.4, 5.3 Hz, 8H), 3.11 (dd, J=13.9, 5.1 Hz, 1H), 3.04 (dd, J=13.9, 7.2 Hz, 1H), 2.85 (s, 2H), 2.74 (h, J=6.4 Hz, 4H), 2.53 (s, 3H), 2.50-2.18 (m, 20H), 2.15 4-2.09 (m, 2H), 1.95 (d, J=17.0 Hz, 1H), 1.75-1.58 (m, 6H), 1.49 (d, J=6.9 Hz, 3H), 1.44-1.36 (m, 3H), 1.03 (s, 9H), 0.99 (s, 3H) ppm. LC/MS (ESI) m/z 1718.7; [M+H]⁺ calcd for C₈₆H₁₀₈ClF₃N₁₃O₁₁S₄⁺: 1718.68.

5-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)picolinamide (compound #6) (3.6 mg, 29% yield). ¹H NMR (600 MHz, CDCl₃) δ 8.69 (d, J=3.0 Hz, 1H), 8.34 (d, J=2.2 Hz, 1H), 8.15-8.09 (m, 2H), 7.97 (d, J=8.8 Hz, 1H), 7.87 (t, J=6.1 Hz, 1H), 7.72 (dd, J=9.1, 2.1 Hz, 2H), 7.50−7.44 (m, 1H), 7.43-7.35 (m, 6H), 7.35-7.24 (m, 5H), 7.15 (d, J=9.0 Hz, 1H), 7.02-6.97 (m, 2H), 6.78 (d, J=9.0 Hz, 2H), 6.61 (d, J=9.4 Hz, 1H), 6.48 (t, J=8.8 Hz, 1H), 5.09 (p, J=7.1 Hz, 1H), 4.73-4.67 (m, 1H), 4.61 (d, J=9.0 Hz, 1H), 4.49 (s, 1H), 4.07 (d, J=11.4 Hz, 1H), 3.90 (s, OH), 3.67 (q, J=5.9 Hz, 4H), 3.58 (dd, J=11.4, 3.5 Hz, 1H), 3.47-3.40 (m, 3H), 3.28 (dt, J=23.1, 5.3 Hz, 8H), 3.11 (dd, J=13.9, 5.0 Hz, 1H), 3.03 (dd, J=13.9, 7.2 Hz, 1H), 2.84 (s, 2H), 2.79-2.68 (m, 4H), 2.53 (d, J=4.9 Hz, 3H), 2.48-2.18 (m, 18H), 2.10 (dd, J=13.5, 8.2 Hz, 2H), 1.95 (d, J=17.8 Hz, 1H), 1.72-1.58 (m, 8H), 1.53-1.30 (m, 10H), 1.04 (s, 9H), 0.99 (s, 3H) ppm. LC/MS (ESI) m/z 1746.8; [M+H]⁺ calcd for C₈₈H₁₁₂ClF₃N₁₃O₁₁S₄⁺: 1746.71.

### Example 3: Preparation of Compounds #7-9

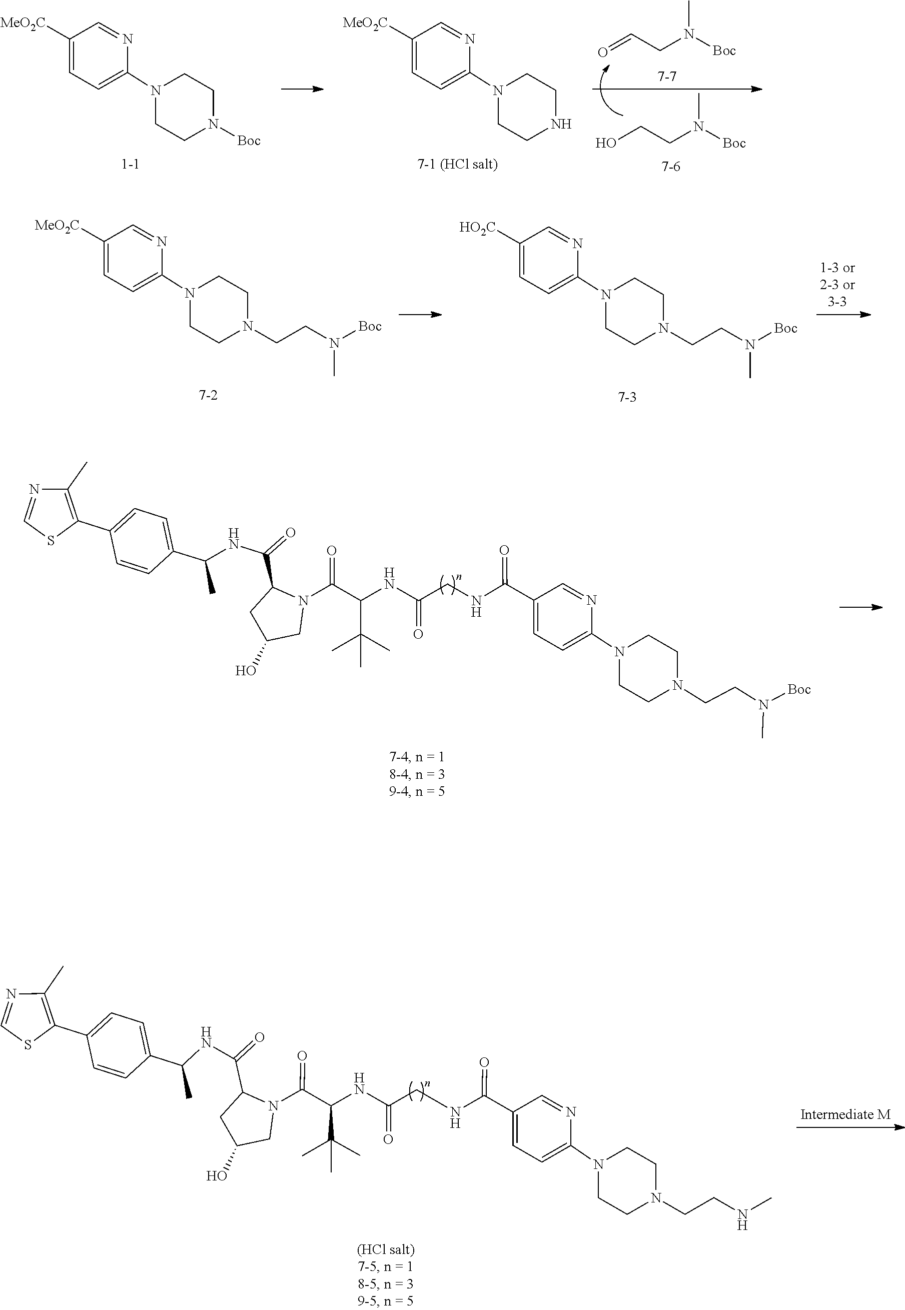

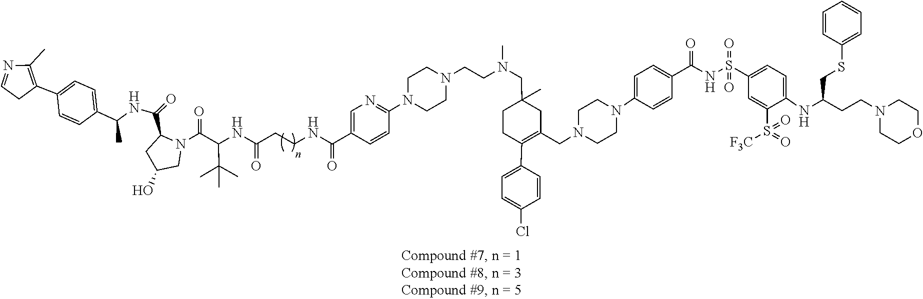

Preparation of ethyl 6-(piperazin-1-yl)nicotinate hydrochloride (7-1): To a solution of 1-1 in DCM was added HCl dioxane solution (4N), then stirred at room temperature for 1 hour. The reaction mixture was concentrated to afford 7-1 (145 mg, quantitative yield) an HCl salt, which was used directly in the next step. LC/MS (ESI) m/z 222.2; [M+H]⁺ calcd for C₁₁H₁₆N₃O₂⁺: 222.12.

Preparation of ethyl 6-(4-(2-((tert-butoxycarbonyl)(methyl)amino)ethyl)piperazin-1-yl)nicotinate (7-2): Under an argon atmosphere, to a solution of DMSO (1.22 mL, 17.14 mmol) in DCM (15 mL) was added oxalyl chloride (966 μL, 11.43 mmol) dropwise at −78° C. with stirring. The resulting mixture was stirred at −78° C. for an additional 15 min. Then a solution of 7-6 (1 g, 5.71 mmol) in DCM (10 mL) was added dropwise. The mixture was stirred at −78° C. for an extra 20 min after completing the addition. TEA (4.8 mL, 34.29 mmol) was added to the mixture at −78° C., then dry-ice/acetone bath was removed. The resulting mixture was warmed up to room temperature and stirred for an additional 30 min. The reaction mixture was added water and extracted with DCM. The combined organic layers were washed with 10% aq. Na₂S₂O₃ and sat. aq. NH₄Cl, dried over Na₂SO₄, filtered, and concentrated to afford aldehyde 7−7, which was used directly in the next step.

A suspension of 7-7 (1.0 equiv.), 7-1 (1.2 equiv.), and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by flash column chromatography to afford 7−2 (161 mg, 76% yield). LC/MS (ESI) m/z 379.1; [M+H]⁺ calcd for C₁₉H₃₁N₄O₄⁺: 379.23.

Preparation of 6-(4-(2-((tert-butoxycarbonyl)(methyl)amino)ethyl)piperazin-1-yl)nicotinic acid (7−3): To a solution of 7-2 (1.0 equiv.) in MeOH/THF was added a solution of LiOH·H₂O (5 equiv.) in water, then stirred at 40° C. overnight. The reaction mixture was concentrated to remove the organic solvents, then adjusted to pH=5-6 with 10% aq. citric acid and extracted with EtOAc. The combined organic layers were washed with brine, dried over Na₂SO₄, filtered, and concentrated to afford 7-3 (135 mg, 93% yield). LC/MS (ESI) m/z 365.1; [M+H]⁺ calcd for C₁₈H₂₉N₄O₄⁺: 365.22.

Preparation of tert-butyl (2-(4-(5-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)pyridin-2-yl)piperazin-1-yl)ethyl)(methyl)carbamate (7-4), tert-butyl (2-(4-(5-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)carbamoyl)pyridin-2-yl)piperazin-1-yl)ethyl)(methyl)carbamate (8-4), and tert-butyl (2-(4-(5-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)pyridin-2-yl)piperazin-1-yl)ethyl)(methyl)carbamate (9-4): A solution of 1-3, 2-3, or 3-3 (1.0 equiv.) and DIPEA (5.0 equiv.) in DMF was added to a solution of 7-3 (1.0 equiv.) and HATU (1.2 equiv.) in DMF. The resulting mixture was stirred at room temperature for 2 hours, then diluted with EtOAc, and washed with sat. aq. NH₄Cl. The organic layer was dried over Na₂SO₄, filtered, and concentrated. The residue was purified by flash column chromatography to afford 7−4, 8-4, and 9-4, respectively. 7-4 (24 mg, 71% yield): ¹H NMR (600 MHz, Chloroform-d) δ 8.75 (s, 1H), 8.68 (s, 1H), 8.20 (s, 1H), 8.03-7.89 (m, 2H), 7.75 (s, 1H), 7.36 (s, 4H), 6.59 (d, J=9.1 Hz, 1H), 5.11 (p, J=7.0 Hz, 1H), 4.80 (t, J=8.4 Hz, 1H), 4.69 (d, J=9.4 Hz, 1H), 4.48-4.36 (m, 2H), 4.03 (dd, J=17.3, 3.9 Hz, 1H), 3.76 (d, J=11.1 Hz, 1H), 3.69-3.59 (m, 6H), 3.53 (d, J=10.7 Hz, 1H), 3.43-3.32 (m, 2H), 2.89 (s, 3H), 2.68-2.53 (m, 5H), 2.52 (s, 3H), 2.25 (dd, J=13.4, 7.7 Hz, 1H), 2.18-2.11 (m, 1H), 1.48-1.37 (m, 12H), 1.04 (s, 9H) ppm. LC/MS (ESI) m/z 848.5; [M+H]⁺ calcd for C₄₃H₆₂N₉O₇S⁺: 848.45. 8-4 (17.8 mg, 53% yield): ¹H NMR (600 MHz, Chloroform-d) δ 8.68 (s, 1H), 8.57 (d, J=2.4 Hz, 1H), 7.94 (dd, J=8.9, 2.5 Hz, 1H), 7.52 (s, 1H), 7.46 (d, J=7.8 Hz, 1H), 7.41-7.33 (m, 4H), 7.05 (d, J=8.3 Hz, 1H), 6.62 (d, J=9.0 Hz, 1H), 5.08 (p, J=7.1 Hz, 1H), 4.66 (t, J=8.2 34 Hz, 1H), 4.53 (d, J=8.4 Hz, 1H), 4.50 (p, J=2.0 Hz, 1H), 4.06 (d, J=11.1 Hz, 1H), 3.68-3.56 (m, 5H), 3.49-3.32 (m, 3H), 2.95-2.84 (m, 4H), 2.64-2.47 (m, 10H), 2.43-2.27 (m, 3H), 2.13 (ddt, J=13.6, 7.9, 1.9 Hz, 1H), 1.89 (p, J=6.7 Hz, 2H), 1.52-1.42 (m, 12H), 1.05 (s, 9H) ppm. LC/MS (ESI) m/z 876.3; [M+H]⁺ calcd for C₄₅H₆₆N₉O₇S⁺: 876.48. 9-4 (19.7 mg, 61% yield): ¹H NMR (600 MHz, Chloroform-d) δ 8.67 (s, 1H), 8.61 (d, J=2.4 Hz, 1H), 7.96 (dd, J=9.0, 2.5 Hz, 1H), 7.55 (d, J=7.8 Hz, 1H), 7.42-7.33 (m, 4H), 6.63 (d, J=9.0 Hz, 1H), 6.58 (d, J=9.1 Hz, 1H), 5.07 (p, J=7.0 Hz, 1H), 4.66 (t, J=8.1 Hz, 1H), 4.60 (d, J=9.1 Hz, 2H), 4.51 6-4.44 (m, 1H), 4.02 (d, J=11.3 Hz, 1H), 3.86-3.49 (m, 10H), 3.39 (q, J=6.6 Hz, 2H), 2.90 (s, 3H), 2.76-2.63 (m, 2H), 2.52 (s, 3H), 2.38-2.30 (m, 1H), 2.30-2.19 (m, 2H), 2.14-2.06 (m, 1H), 1.69-1.55 (m, 4H), 1.45 (s, 9H), 1.41 (d, 3H), 1.40-1.33 (m, 2H), 1.02 (s, 9H) ppm. LC/MS (ESI) m/z 904.6; [M+H]⁺ calcd for C₄₇H₇₀N₉O₇S⁺: 904.51.

Preparation of N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-6-(4-(2-(methylamino)ethyl)piperazin-1-yl)nicotinamide hydrochloride (7-5), N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-6-(4-(2-(methylamino)ethyl)piperazin-1-yl)nicotinamide hydrochloride (8-5), and N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-6-(4-(2-(methylamino)ethyl)piperazin-1-yl)nicotinamide hydrochloride (9-5): To a solution of 7-4, 8-4, or 9-4 in DCM was added HCl dioxane solution (4N), then stirred at room temperature for 1-2 hours. The reaction mixture was concentrated to afford 7-5, 8-5, and 9-5, respectively, as HCl salts, which was used directly in the next step. 7-5 (15 mg, quantitative yield): LC/MS (ESI) m/z 748.4; [M+H]⁺ calcd for C₃₈H₅₄N₉O₅S⁺: 748.40. 8-5 (18 mg, quantitative yield): LC/MS (ESI) m/z 776.3; [M+H]⁺ calcd for C₄₀H₅₈N₉O₅S⁺: 776.43. 9-5 (17 mg, quantitative yield): LC/MS (ESI) m/z 804.4; [M+H]⁺ calcd for C₄₂H₆₂N₉O₅S⁺: 804.46.

Preparation of 6-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)nicotinamide (compound #7), 6-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)nicotinamide (compound #8), and 6-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)nicotinamide (compound #9): A suspension of Intermediate M (1.0 equiv.), 7-5/8-5/9-5 (1.2-1.6 equiv.,) TEA (20.0 equiv.) and NaBH(OAc)₃ (2.0 equiv.) in DCE was stirred at room temperature overnight. The reaction mixture was washed with sat. aq. NH₄Cl, dried over Na₂SO₄, filtered, and concentrated. The residue was purified by preparative TLC (DCM/MeOH) to afford the desired compound. Compound #7 (3.1 mg, 18% yield): ¹H NMR (600 MHz, CDCl₃) δ 8.67 (s, 1H), 8.54 (s, 1H), 8.33 (d, J=2.3 Hz, 1H), 8.05 (d, J=9.2 Hz, 1H), 7.84-7.70 (m, 3H), 7.40−7.31 (m, 6H), 7.31-7.20 (m, 5H), 7.01-6.96 (m, 2H), 6.70 (d, J=8.5 Hz, 2H), 6.58 (d, J=9.3 Hz, 1H), 6.30 (s, 1H), 5.06 (q, J=6.9 Hz, 1H), 4.71 (s, 1H), 4.56 (s, 1H), 4.47 (s, 1H), 4.21-3.99 (m, 3H), 3.86 (s, 1H), 3.68-3.60 (m, 6H), 3.53 (s, 4H), 3.13 (d, J=53.8 Hz, 5H), 3.01 (dd, J=13.8, 7.2 Hz, 1H), 2.83 (s, 2H), 2.71 (s, 2H), 2.59 (s, 6H), 2.51 (s, 3H), 2.47-2.19 (m, 22H), 2.18-2.07 (m, 2H), 1.98 (d, J=17.4 Hz, 1H), 1.68-1.54 (m, 3H), 1.47-1.35 (m, 4H), 1.05 (s, 9H), 0.96 (s, 3H) ppm. LC/MS (ESI) m/z 1719.8; [M+H]⁺ calcd for C₈₅H₁₀₇ClF₃N₁₄O₁₁S₄⁺: 1719.68.

Compound #8 (4.8 mg, 27% yield): ¹H NMR (600 MHz, CDCl₃) δ 8.67 (s, 1H), 8.51 (s, 1H), 8.33 (s, 1H), 8.05 (d, J=9.2 Hz, 1H), 7.82 (d, J=9.2 Hz, 1H), 7.77 (d, J=8.3 Hz, 2H), 7.51-7.33 (m, 7H), 7.32-7.20 (m, 4H), 7.01-6.97 (m, 2H), 6.70 (d, J=8.6 Hz, 2H), 6.57 (d, J=9.3 26 Hz, 1H), 6.32 (s, 1H), 5.08 (p, J=7.3 Hz, 1H), 4.66 (q, J=8.1 Hz, 1H), 4.50 (s, 2H), 4.08 (d, J=11.4 Hz, 1H), 3.86 (s, 1H), 3.69-3.56 (m, 5H), 3.55-3.42 (m, 5H), 3.39 (s, 1H), 3.17 (d, J=5.0 Hz, 4H), 3.10 (dd, J=13.9, 4.8 Hz, 1H), 3.00 (dd, J=13.8, 7.2 Hz, 1H), 2.84 (t, J=13.0 Hz, 2H), 2.70 (d, J=7.5 Hz, 2H), 2.58 (d, J=8.1 Hz, 6H), 2.52 (s, 4H), 2.48-2.20 (m, 22H), 2.16-2.07 (m, 3H), 2.01-1.88 (m, 2H), 1.84-1.55 (m, 4H), 1.48 (dd, J=12.5, 6.9 Hz, 3H), 1.39 (s, 1H), 1.05 (s, 9H), 0.96 (s, 3H) ppm. LC/MS (ESI) m/z 1747.9; [M+H]⁺ calcd for Cs₇H₁₁₁ClF₃N₁₄O₁₁S₄⁺: 1747.71.

Compound #9 (4.1 mg, 23% yield): ¹H NMR (600 MHz, CDCl₃) δ 8.68 (s, 1H), 8.54 (s, 1H), 8.34 (d, J=2.1 Hz, 1H), 8.07 (d, J=9.1 Hz, 1H), 7.86 (d, J=9.0 Hz, 1H), 7.76 (d, J=8.4 Hz, 2H), 7.45 (d, J=6.9 Hz, 1H), 7.42-7.33 (m, 6H), 7.33-7.22 (m, 5H), 7.00 (d, J=8.2 Hz, 2H), 6.71 (d, J=8.6 Hz, 2H), 6.57 (dd, J=24.8, 9.0 Hz, 2H), 6.36 (s, 1H), 5.07 (p, J=7.1 Hz, 1H), 4.66 (q, J=7.9 Hz, 1H), 4.57 (d, J=8.6 Hz, 1H), 4.47 (s, 1H), 4.05 (d, J=11.3 Hz, 1H), 3.88 (s, 1H), 3.66 (s, 5H), 3.57 (dd, J=11.3, 3.3 Hz, 1H), 3.50 (s, 4H), 3.45-3.39 (m, 2H), 3.19 (s, 4H), 3.11 (dd, J=13.9, 4.9 Hz, 1H), 3.01 (dd, J=13.8, 7.3 Hz, 1H), 2.85 (s, 2H), 2.71 (s, 2H), 2.58 (s, 6H), 2.52 (s, 3H), 2.46-2.17 (m, 22H), 2.11 (d, J=10.4 Hz, 2H), 1.98 (s, 1H), 1.74-1.43 (m, 10H), 1.39 (q, J=7.6 Hz, 2H), 1.03 (s, 9H), 0.97 (s, 3H) ppm. LC/MS (ESI) m/z 1775.5; [M+H]⁺ calcd for C₈₉H₁₁₅ClF₃N₁₄O₁₁S₄⁺: 1775.74.

### Example 4: Preparation of Compounds #10-12

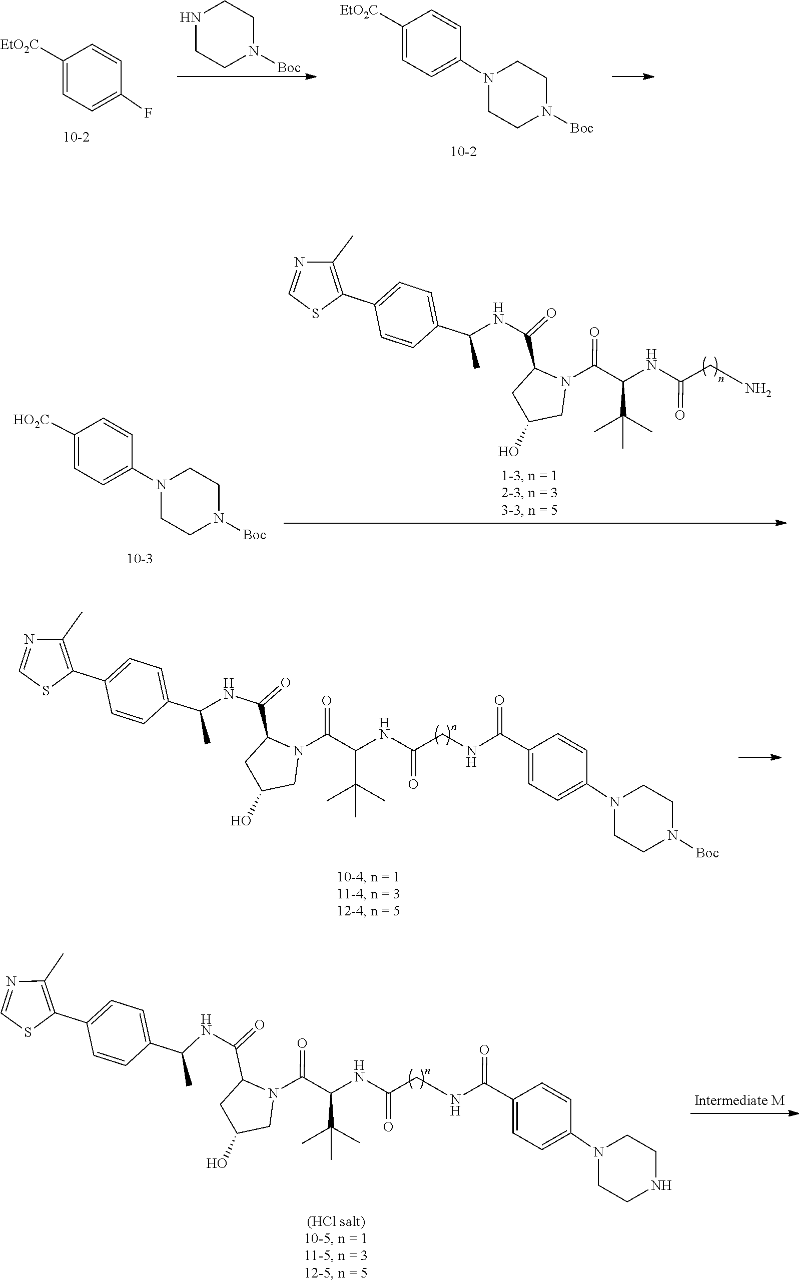

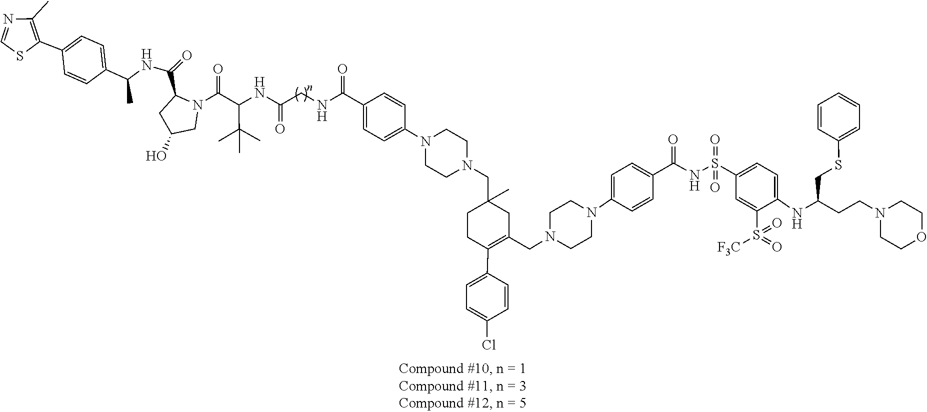

Compounds #10-12 were prepared by following General Procedure A.

tert-butyl 4-(4-(ethoxycarbonyl)phenyl)piperazine-1-carboxylate (10-2) (580 mg, 58% yield). ¹H NMR (600 MHz, CDCl₃) δ 8.00-7.92 (m, 2H), 6.91-6.84 (m, 2H), 4.35 (q, J=7.1 Hz, 2H), 3.60 (t, 4H), 3.32 (t, J=5.1 Hz, 4H), 1.51 (s, 9H), 1.39 (t, J=7.1 Hz, 3H) ppm. LC/MS (ESI) m/z 335.3; [M+H]⁺ calcd for C₁₈H₂₇N₂O₄⁺: 335.20.

4-(4-(tert-butoxycarbonyl)piperazin-1-yl)benzoic acid (10-3) (250 mg, 91% yield). ¹H NMR (600 MHz, DMSO) δ 7.82-7.74 (m, 2H), 7.00-6.93 (m, 2H), 3.46 (t, J=5.2 Hz, 4H), 3.29 (t, 4H), 1.43 (s, 9H) ppm. LC/MS (ESI) m/z 307.2; [M+H]⁺ calcd for C₁₆H₂₃N₂O₄⁺: 307.17.

tert-butyl 4-(4-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)phenyl)piperazine-1-carboxylate (10−4) (23 mg, 74% yield). ¹H NMR (600 MHz, CDCl₃) δ 8.68 (s, 1H), 8.06 (s, 1H), 7.96-7.89 (m, 1H), 7.85 (d, J=8.4 Hz, 2H), 7.60 (s, 1H), 7.39-7.33 (m, 4H), 6.87 (d, J=8.6 Hz, 2H), 5.11 (p, J=7.1 Hz, 1H), 4.81 (t, J=8.5 Hz, 1H), 4.66 (d, J=8.9 Hz, 1H), 4.46 (d, J=17.0 Hz, 1H), 4.38 (s, 1H), 3.96 (d, J=17.0 Hz, 1H), 3.58 (t, J=5.3 Hz, 4H), 3.50 (d, J=11.0 Hz, 1H), 3.26 (t, J=5.3 Hz, 4H), 2.52 (s, 3H), 2.27 (dd, J=13.5, 7.6 Hz, 1H), 2.15 (t, J=10.8 Hz, 1H), 1.50 (s, 9H), 1.44 (dd, J=7.0, 3.4 Hz, 3H), 1.05 (s, 9H) ppm. LC/MS (ESI) m/z 790.3; [M+H]⁺ calcd for C₄₁H₅₆N7O₇S⁺: 790.40.

tert-butyl 4-(4-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)carbamoyl)phenyl)piperazine-1-carboxylate (11-4) (25 mg, 81% yield). ¹H NMR (600 MHz, CDCl₃) δ 8.68 (s, 1H), 7.74-7.71 (m, 2H), 7.47 (d, J=7.8 Hz, 1H), 7.42-7.34 (m, 4H), 7.12 (d, J=8.5 Hz, 1H), 6.92 (t, J=5.8 Hz, 1H), 6.89-6.86 (m, 2H), 5.08 (p, J=7.1 Hz, 1H), 4.69 (t, J=8.0 Hz, 1H), 4.54 (d, J=8.6 Hz, 1H), 4.48 (s, 1H), 4.05 (dt, J=11.6, 1.8 Hz, 1H), 3.63-3.54 (m, 5H), 3.48-3.39 (m, 2H), 3.24 (t, J=5.2 Hz, 4H), 2.52 (s, 3H), 2.45-2.38 (m, 1H), 2.37-2.28 (m, 2H), 2.16-2.07 (m, 2H), 1.98-1.84 (m, 2H), 1.52-1.46 (m, 12H), 1.05 (s, 9H) ppm. LC/MS (ESI) m/z 818.4; [M+H]⁺ calcd for C₄₃H₆₀N₇O₇S⁺: 818.43.

tert-butyl 4-(4-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)phenyl)piperazine-1-carboxylate (12-4) (20 mg, 66% yield). ¹H NMR (600 MHz, CDCl₃) δ 8.68 (s, 1H), 7.72-7.67 (m, 2H), 7.44 (d, J=7.8 Hz, 1H), 7.40 (d, J=8.3 Hz, 2H), 7.36 (d, J=8.3 Hz, 2H), 6.90-6.84 (m, 2H), 6.56 (d, J=9.1 Hz, 1H), 6.50 (t, J=5.8 Hz, 1H), 5.08 (q, J=7.1 Hz, 1H), 4.66 (t, J=8.1 Hz, 1H), 4.59 (d, J=9.2 Hz, 1H), 4.47 (d, J=4.1 Hz, 1H), 4.03 (d, J=11.2 Hz, 1H), 3.57 (q, J=4.2 Hz, 5H), 3.40 (qd, J=7.0, 2.4 Hz, 2H), 3.24 (t, J=5.2 Hz, 4H), 2.53 (s, 3H), 2.40-2.34 (m, 1H), 2.29-2.18 (m, 2H), 2.16 (s, 1H), 2.12-2.05 (m, 1H), 1.70-1.62 (m, 2H), 1.58 (p, J=7.3 Hz, 2H), 1.51-1.45 (m, 12H), 1.37 (qt, J=10.2, 4.6 Hz, 2H), 1.03 (s, 9H) ppm. LC/MS (ESI) m/z 846.5; [M+H]⁺ calcd for C₄₅H₆₄N₇O₇S⁺: 846.46.

(2S,4R)-1-((S)-3,3-dimethyl-2-(2-(4-(piperazin-1-yl)benzamido)acetamido)butanoyl)-4-hydroxy-N—((S)-1-(4-(4-methylthiazol-5-yl)phenyl)ethyl)pyrrolidine-2-carboxamide hydrochloride (10-5) (18 mg, quantitative yield). LC/MS (ESI) m/z 690.5; [M+H]⁺ calcd for C₃₆H₄₈N₇O₅S⁺: 690.34.

(2S,4R)-1-((S)-3,3-dimethyl-2-(4-(4-(piperazin-1-yl)benzamido)butanamido)butanoyl)-4-hydroxy-N—((S)-1-(4-(4-methylthiazol-5-yl)phenyl)ethyl)pyrrolidine-2-carboxamide hydrochloride (11-5) (21 mg, quantitative yield). LC/MS (ESI) m/z 718.3; [M+H]⁺ calcd for C₃₈H₅₂N₇O₅S⁺: 718.37.

(2S,4R)-1-((S)-3,3-dimethyl-2-(6-(4-(piperazin-1-yl)benzamido)hexanamido)butanoyl)-4-hydroxy-N—((S)-1-(4-(4-methylthiazol-5-yl)phenyl)ethyl)pyrrolidine-2-carboxamide hydrochloride (12-5) (20 mg, quantitative yield). LC/MS (ESI) m/z 746.5; [M+H]⁺ calcd for C₄₀H₅₆N₇O₅S⁺: 746.41.

(2S,4R)-1-((2S)-2-(2-(4-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)benzamido)acetamido)-3,3-dimethylbutanoyl)-4-hydroxy-N—((S)-1-(4-(4-methylthiazol-5-yl)phenyl)ethyl)pyrrolidine-2-carboxamide (compound #10) (5.4 mg, 16% yield). ¹H NMR (600 MHz, CDCl₃) δ 8.68 (d, J=6.6 Hz, 1H), 8.34 (s, 1H), 8.08 (s, 1H), 7.74 (d, J=18.7 Hz, 2H), 7.71-7.61 (m, 3H), 7.36 (h, J 24=6.2 Hz, 7H), 7.29 (d, J=8.5 Hz, 4H), 7.03-6.96 (m, 2H), 6.80-6.68 (m, 3H), 6.59 (d, J=9.2 Hz, 1H), 5.10 (q, J=6.9 Hz, 1H), 4.79 (t, J=8.1 Hz, 1H), 4.61 (d, J=11.0 Hz, 1H), 4.48 (s, 1H), 4.17 (s, 2H), 4.03 (s, 1H), 3.88 (s, 1H), 3.70-3.59 (m, 6H), 3.22 (s, 9H), 3.12-3.08 (m, 1H), 3.01 (dd, J=13.8, 7.2 Hz, 1H), 2.90-2.79 (m, 2H), 2.77-2.65 (m, 4H), 2.52 (s, 3H), 2.49 28-2.16 (m, 20H), 2.10 (d, J=9.0 Hz, 1H), 1.88 (s, 1H), 1.71-1.59 (m, 2H), 1.52-1.30 (m, 4H), 1.06 (s, 9H), 0.98 (d, J=2.1 Hz, 3H) ppm. LC/MS (ESI) m/z 1661.5; [M+H]⁺ calcd for C₈₃H₁₀₁ClF₃N₁₂O₁₁S₄⁺: 1661.62.

(2S,4R)-1-((2S)-2-(4-(4-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)benzamido)butanamido)-3,3-dimethylbutanoyl)-4-hydroxy-N—((S)-1-(4-(4-methylthiazol-5-yl)phenyl)ethyl)pyrrolidine-2-carboxamide (compound #11) (3.6 mg, 11% yield). ¹H NMR (600 MHz, CDCl₃) δ 8.68 (s, 1H), 8.34 (d, J=2.3 Hz, 1H), 8.12 (d, J=9.3 Hz, 1H), 7.75-7.64 (m, 4H), 7.48-7.34 (m, 7H), 7.34 4-7.23 (m, 4H), 7.08-6.96 (m, 3H), 6.85 (d, J=8.4 Hz, 2H), 6.75 (d, J=8.7 Hz, 2H), 6.62 (d, J=9.4 Hz, 1H), 5.08 (p, J=7.1 Hz, 1H), 4.74-4.66 (m, 1H), 4.54 (d, J=8.1 Hz, 1H), 4.48 (s, 1H), 4.06 (d, J=11.3 Hz, 1H), 3.90 (s, 1H), 3.72-3.62 (m, 5H), 3.62-3.56 (m, 1H), 3.50-3.42 (m, 3H), 3.25 (s, 8H), 3.11 (dd, J=14.0, 5.0 Hz, 1H), 3.03 (dd, J=13.9, 7.2 Hz, 1H), 2.85 (s, 2H), 2.77-2.65 (m, 4H), 2.52 (s, 3H), 2.50-2.21 (m, 20H), 2.10 (dd, J=13.7, 8.4 Hz, 2H), 1.92 (d, J=15.9 Hz, 2H), 1.73-1.62 (m, 2H), 1.57-1.35 (m, 4H), 1.05 (d, J=2.2 Hz, 9H), 0.98 (s, 3H) ppm. LC/MS (ESI) m/z 1689.3; [M+H]⁺ calcd for C₈₅H₁₀₅ClF₃N₁₂O₁₁S₄⁺: 1689.65.

(2S,4R)-1-((2S)-2-(6-(4-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)benzamido)hexanamido)-3,3-dimethylbutanoyl)-4-hydroxy-N—((S)-1-(4-(4-methylthiazol-5-yl)phenyl)ethyl)pyrrolidine-2-carboxamide (compound #12) (4.1 mg, 12% yield). ¹H NMR (600 MHz, CDCl₃) δ 8.68 (s, 1H), 8.38-8.31 (m, 1H), 8.10 (d, J=9.1 Hz, 1H), 7.75 (d, J=8.4 Hz, 2H), 7.67 (d, J=8.5 Hz, 2H), 7.43-7.34 (m, 7H), 7.34-7.22 (m, 4H), 7.03-6.96 (m, 2H), 6.81 (d, J=8.4 Hz, 2H), 6.75 (d, J=8.6 Hz, 2H), 6.59 (d, J=9.3 Hz, 1H), 6.53 (d, J=8.8 Hz, 1H), 5.07 (p, J=7.0 Hz, 1H), 4.66 (t, J=8.2 Hz, 1H), 4.57 (d, J=8.9 Hz, 1H), 4.46 (s, 1H), 4.05 (d, J=11.6 Hz, 1H), 3.88 (s, 1H), 3.66 (q, J=6.3 Hz, 5H), 3.55 (dd, J=10.0, 5.6 Hz, 1H), 3.46-3.38 (m, 2H), 3.22 (d, J=5.1 Hz, 7H), 3.11 (dd, J=13.8, 4.9 Hz, 1H), 3.02 (dd, J=13.9, 7.2 Hz, 1H), 2.85 (p, J=12.3 Hz, 2H), 2.71 (dq, J=11.6, 5.7 Hz, 4H), 2.52 (s, 3H), 2.48-2.21 (m, 22H), 2.11 (d, J=9.4 Hz, 2H), 1.88 (d, J=17.3 Hz, 1H), 1.78-1.31 (m, 12H), 1.02 (s, 9H), 0.98 (s, 3H) ppm. LC/MS (ESI) m/z 1717.4; [M+H]⁺ calcd for C₈₇H₁₀₉ClF₃N₁₂O₁₁S₄⁺: 1717.69.

### Example 5: Preparation of Compounds #13-15

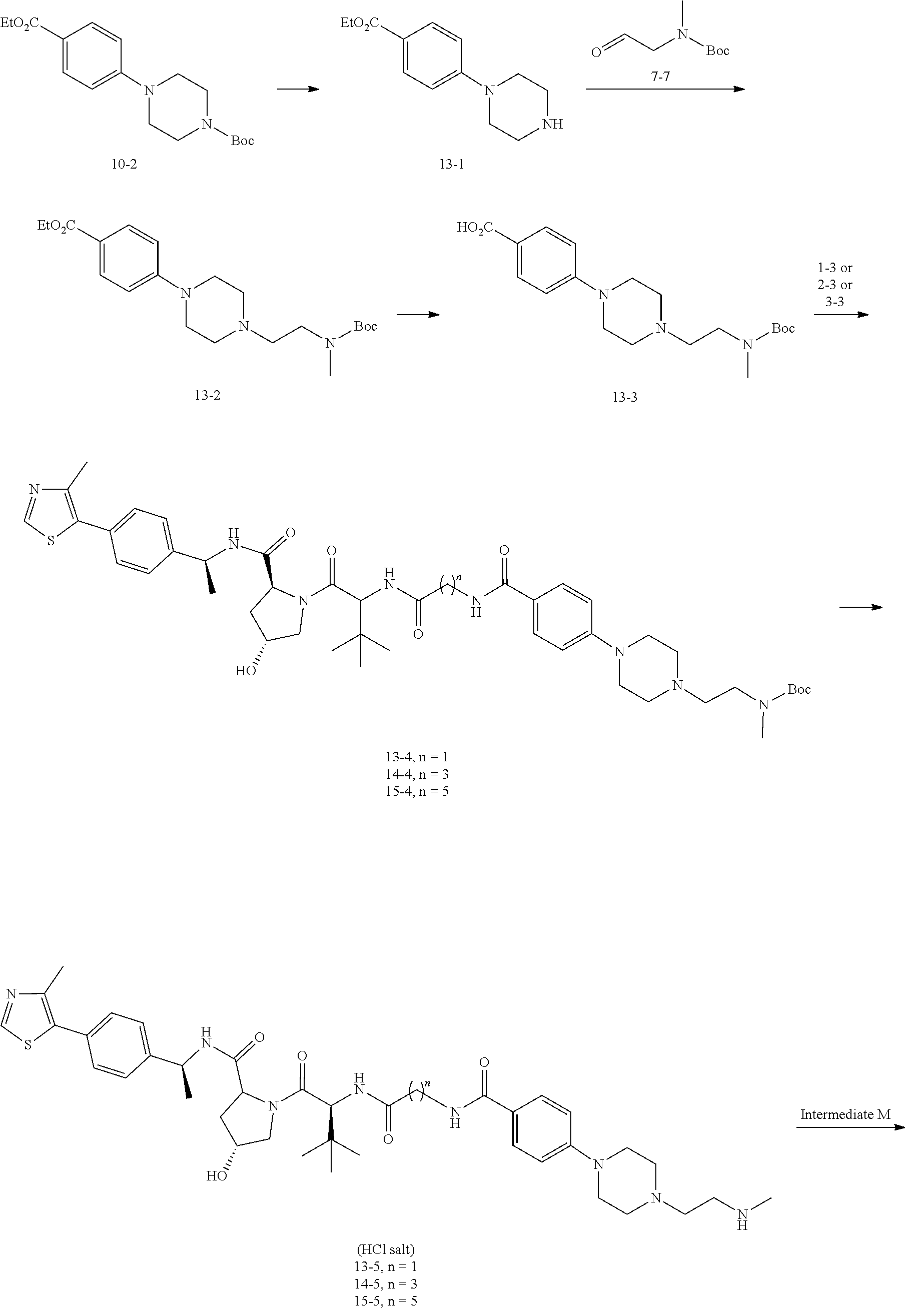

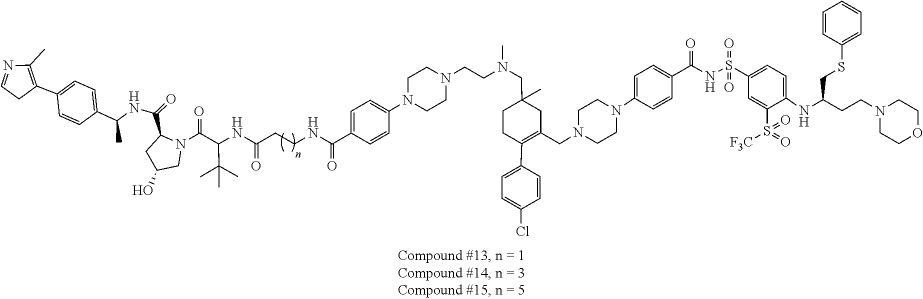

Compounds #13-15 were prepared by following the procedure similar to the preparation of compounds #7-9.

ethyl 4-(piperazin-1-yl)benzoate (13-1) (306 mg, quantitative yield). LC/MS (ESI) m/z 235.2; [M+H]⁺ calcd for C₁₃H₁₉N₂O₂⁺: 235.14.

ethyl 4-(4-(2-((tert-butoxycarbonyl)(methyl)amino)ethyl)piperazin-1-yl)benzoate (13−2) (296 mg, 66% yield). LC/MS (ESI) m/z 392.4; [M+H]⁺ calcd for C₂₁H₃₄N₃O₄⁺: 392.25.

4-(4-(2-((tert-butoxycarbonyl)(methyl)amino)ethyl)piperazin-1-yl)benzoic acid (13-3) (180 mg, 97% yield). ¹H NMR (600 MHz, DMSO) δ 7.79-7.72 (m, 2H), 7.01-6.92 (m, 2H), 3.42-3.24 (m, 6H), 2.80 (d, J=11.1 Hz, 3H), 2.55 (q, J=7.5 Hz, 4H), 2.45 (d, J=6.5 Hz, 2H), 1.40 (s, 9H) ppm. LC/MS (ESI) m/z 364.3; [M+H]⁺ calcd for C₁₉H₃₀N₃O₄⁺: 364.22.

tert-butyl (2-(4-(4-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)phenyl)piperazin-1-yl)ethyl)(methyl)carbamate (13−4) (25.2 mg, 72% yield). ¹H NMR (600 MHz, CDCl₃) δ 8.67 (s, 1H), 8.16 (d, J=7.8 Hz, 1H), 7.93 (d, J=9.0 Hz, 1H), 7.81 (s, 2H), 7.73 (s, 1H), 7.39-7.31 (m, 4H), 6.84 (s, 2H), 5.09 (p, J=7.0 Hz, 1H), 4.81 (t, J=8.4 Hz, 1H), 4.70 (d, J=9.3 Hz, 1H), 4.41 (d, J=17.7 Hz, 2H), 4.04 (dd, J=17.3, 4.0 Hz, 1H), 3.84-3.73 (m, 1H), 3.57-3.44 (m, 2H), 3.43-3.27 (m, 5H), 2.90 (s, 3H), 2.83 (s, 2H), 2.70 (s, 2H), 2.63-2.55 (m, 1H), 2.50 (s, 3H), 2.26 (dd, J=13.4, 7.6 Hz, 1H), 2.18-2.08 (m, 1H), 1.48-1.39 (m, 12H), 1.03 (s, 9H) ppm. LC/MS (ESI) m/z 847.5; [M+H]⁺ calcd for C₄₄H₆₃N₈O₇S⁺: 847.45.

tert-butyl (2-(4-(4-((4-(((S)-3,3-dimethyl-1-((2S,4R)-4-methyl-2-(((S)-1-(4-(4-methylthiazol-5-yl)phenyl)ethyl)carbamoyl)pyrrolidin-1-yl)-1-oxobutan-2-yl)amino)-4-oxobutyl)carbamoyl)phenyl)piperazin-1-yl)ethyl)(methyl)carbamate (14−4) (27.7 mg, 77% yield). ¹H NMR (600 MHz, CDCl₃) δ 8.69 (s, 1H), 8.55 (d, J=4.3 Hz, 1H), 8.22 (d, J=8.4 Hz, 1H), 7.74-7.63 (m, 2H), 7.54 (s, 1H), 7.38 (q, 4H), 7.30-7.24 (m, 1H), 6.80 (s, 2H), 5.08 (p, J=7.0 Hz, 1H), 4.71 (t, J=8.0 Hz, 1H), 4.57 (d, J=7.6 Hz, 1H), 4.50 (s, 1H), 4.03 (d, J=11.2 Hz, 1H), 3.65-3.31 (m, 8H), 3.10 (s, 2H), 3.05-2.72 (m, 6H), 2.52 (s, 3H), 2.48-2.20 (m, 4H), 2.12 (dd, J=13.5, 8.3 Hz, 1H), 1.93-1.83 (m, 2H), 1.53-1.41 (m, 12H), 1.05 (s, 9H) ppm. LC/MS (ESI) m/z 875.5; [M+H]⁺ calcd for C₄₆H₆₇N₈O₇S⁺: 875.48.

tert-butyl (2-(4-(4-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)phenyl)piperazin-1-yl)ethyl)(methyl)carbamate (15-4) (25.7 mg, 69% yield). ¹H NMR (600 MHz, CDCl₃) δ 8.68 (s, 1H), 7.69 (d, J=8.5 Hz, 2H), 7.45 (d, J=7.8 Hz, 1H), 7.42-7.34 (m, 4H), 6.86 (d, J=8.5 Hz, 2H), 6.56 (d, J=9.1 Hz, 1H), 6.50 (t, J=5.8 Hz, 1H), 5.08 (p, J=7.1 Hz, 1H), 4.68 (t, J=8.1 Hz, 1H), 4.60 (d, J=9.1 Hz, 1H), 4.47 (d, 1H), 4.03 (d, J=1.8 Hz, 1H), 3.57 (dd, J=11.3, 3.5 Hz, 1H), 3.46-3.31 (m, 4H), 3.27 (t, J=5.1 Hz, 4H), 2.89 (s, 3H), 2.65 (s, 4H), 2.61-2.51 (m, 5H), 2.41-2.34 (m, 1H), 2.32-2.16 (m, 2H), 2.13-2.06 (m, 1H), 1.72-1.62 (m, 2H), 1.58 (p, J=7.3 Hz, 2H), 1.48-1.44 (m, 12H), 1.40-1.32 (m, 2H), 1.03 (s, 9H) ppm. LC/MS (ESI) m/z 903.5; [M+H]⁺ calcd for C₄₈H₇₁N₈O₇S⁺: 903.52.

(2S,4R)-1-((S)-3,3-dimethyl-2-(2-(4-(4-(2-(methylamino)ethyl)piperazin-1-yl)benzamido)acetamido)butanoyl)-4-hydroxy-N—((S)-1-(4-(4-methylthiazol-5-yl)phenyl)ethyl)pyrrolidine-2-carboxamide hydrochloride (13-5) (22 mg, quantitative yield). LC/MS (ESI) m/z 747.5; [M+H]⁺ calcd for C₃₉H₅₅N₈O₅S⁺: 747.40.

(2S,4R)-1-((S)-3,3-dimethyl-2-(4-(4-(4-(2-(methylamino)ethyl)piperazin-1-yl)benzamido)butanamido)butanoyl)-4-hydroxy-N—((S)-1-(4-(4-methylthiazol-5-yl)phenyl)ethyl)pyrrolidine-2-carboxamide hydrochloride (14-5) (18 mg, quantitative yield). LC/MS (ESI) m/z 775.3; [M+H]⁺ calcd for C₄₁H₅₉N₈O₅S⁺: 775.43.

(2S,4R)-1-((S)-3,3-dimethyl-2-(6-(4-(4-(2-(methylamino)ethyl)piperazin-1-yl)benzamido)hexanamido)butanoyl)-4-hydroxy-N—((S)-1-(4-(4-methylthiazol-5-yl)phenyl)ethyl)pyrrolidine-2-carboxamide hydrochloride (15-5) (19 mg, quantitative yield). LC/MS (ESI) m/z 803.5; [M+H]⁺ calcd for C₄₃H₆₃N₈O₅S⁺: 803.46.

(2S,4R)-1-((2S)-2-(2-(4-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)benzamido)acetamido)-3,3-dimethylbutanoyl)-4-hydroxy-N—((S)-1-(4-(4-methylthiazol-5-yl)phenyl)ethyl)pyrrolidine-2-carboxamide (compound #13) (5.5 mg, 32% yield). ¹H NMR (600 MHz, CDCl₃) δ 8.67 (d, J=1.0 Hz, 1H), 8.34 (d, J=2.2 Hz, 1H), 8.08 (d, J=9.1 Hz, 1H), 7.81-7.74 (m, 2H), 7.64 (dd, J=8.9, 3.6 Hz, 2H), 7.40-7.32 (m, 7H), 7.32-7.26 (m, 4H), 7.26-7.22 (m, 1H), 7.00 (d, J=8.1 Hz, 2H), 6.71-6.62 (m, 3H), 6.60 (d, J=9.4 Hz, 1H), 5.10-5.02 (m, 1H), 4.73 (td, J=8.1, 3.8 Hz, 1H), 4.61 (s, 1H), 4.47 (s, 1H), 4.16 (s, 2H), 4.03 (d, J=11.3 Hz, 1H), 3.88 (s, 1H), 3.73-3.58 (m, 5H), 3.19 (s, 8H), 3.10 (dd, J=13.6, 4.7 Hz, 1H), 3.01 (dd, J=13.8, 7.2 Hz, 1H), 2.94-2.77 (m, 3H), 2.70 (s, 6H), 2.57-2.21 (m, 25H), 2.20-2.01 (m, 3H), 1.70-1.50 (m, 4H), 1.44 (t, J=6.6 Hz, 3H), 1.34-1.19 (m, 1H), 1.06 (s, 9H), 1.00 (s, 3H) ppm. LC/MS (ESI) m/z 1718.8; [M+H]⁺ calcd for C₈₆H₁₀₈ClF₃N₁₃O₁₁S₄⁺: 1718.68.

(2S,4R)-1-((2S)-2-(4-(4-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)benzamido)butanamido)-3,3-dimethylbutanoyl)-4-hydroxy-N—((S)-1-(4-(4-methylthiazol-5-yl)phenyl)ethyl)pyrrolidine-2-carboxamide (compound #14) (4.9 mg, 28% yield). ¹H NMR (600 MHz, CDCl₃) δ 8.68 (s, 1H), 8.34 (d, J=2.5 Hz, 1H), 8.10-8.05 (m, 1H), 7.78-7.73 (m, 2H), 7.63 (d, J=8.4 Hz, 2H), 7.41-7.32 (m, 7H), 7.33-7.22 (m, 5H), 7.01-6.98 (m, 2H), 6.69 (dd, J=9.0, 5.7 Hz, 4H), 6.59 (d, J=9.3 Hz, 1H), 5.07 (td, J=6.7, 3.0 Hz, 1H), 4.68 (td, J=8.1, 3.9 Hz, 1H), 4.53 (t, J=4.3 Hz, 1H), 4.48 (s, 1H), 4.05 (d, J=11.3 Hz, 1H), 3.88 (s, 1H), 3.71-3.56 (m, 5H), 3.51-3.42 (m, 3H), 3.24-3.14 (m, 8H), 3.11 (dd, J=13.8, 4.9 Hz, 1H), 3.01 (dd, J=13.9, 7.3 Hz, 1H), 2.84 (s, 2H), 2.79-2.59 (m, 8H), 2.52 (s, 3H), 2.48-2.19 (m, 22H), 2.12 (d, J=10.6 Hz, 2H), 2.03-1.96 (m, 1H), 1.96-1.88 (m, 2H), 1.76-1.27 (m, 6H), 1.05 (s, 9H), 0.98 (s, 3H) ppm. LC/MS (ESI) m/z 1746.6; [M+H]⁺ calcd for C₈₈H₁₁₂ClF₃N₁₃O₁₁S₄⁺: 1746.71.

(2S,4R)-1-((2S)-2-(6-(4-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)benzamido)hexanamido)-3,3-dimethylbutanoyl)-4-hydroxy-N—((S)-1-(4-(4-methylthiazol-5-yl)phenyl)ethyl)pyrrolidine-2-carboxamide (compound #15) (6.6 mg, 37% yield). ¹H NMR (600 MHz, CDCl₃) δ 8.68 (s, 1H), 8.37-8.32 (m, 1H), 8.06-8.00 (m, 1H), 7.83 (d, J=8.5 Hz, 2H), 7.68 (d, J=8.4 Hz, 2H), 7.45 (d, J=8.0 Hz, 1H), 7.41-7.33 (m, 6H), 7.28 (t, J=7.6 Hz, 4H), 7.23 (t, J=7.3 Hz, 1H), 7.01 (dd, J=8.4, 2.2 Hz, 2H), 6.93-6.79 (m, 1H), 6.71 (dd, J=23.6, 8.5 Hz, 4H), 6.59 (d, J=9.0 Hz, 1H), 6.55 (d, J=9.3 Hz, 1H), 5.07 (p, J=7.1 Hz, 1H), 4.67 (t, J=8.1 Hz, 1H), 4.60 (d, J=9.1 Hz, 1H), 4.47 (s, 1H), 4.04 (d, J=11.3 Hz, 1H), 3.85 (s, 1H), 3.71-3.61 (m, 5H), 3.57 (dd, J=11.5, 3.4 Hz, 1H), 3.42 (d, J=12.0 Hz, 2H), 3.21 (t, J=5.2 Hz, 8H), 3.12 (q, J=7.3 Hz, 1H), 2.99 (dd, J=13.8, 7.3 Hz, 1H), 2.96-2.83 (m, 3H), 2.71 (s, 5H), 2.58-2.19 (m, 25H), 2.10 (dd, J=13.6, 8.0 Hz, 2H), 1.71-1.57 (m, 5H), 1.54 (t, J=7.4 Hz, 1H), 1.47 (dd, J=10.3, 6.8 Hz, 7H), 1.02 (d, J=4.4 Hz, 12H) ppm. LC/MS (ESI) m/z 1774.6; [M+H]⁺ calcd for C₉₀H₁₁₆ClF₃N₁₃O₁₁S₄⁺: 1774.74.

### Example 6: Preparation of Compound #16

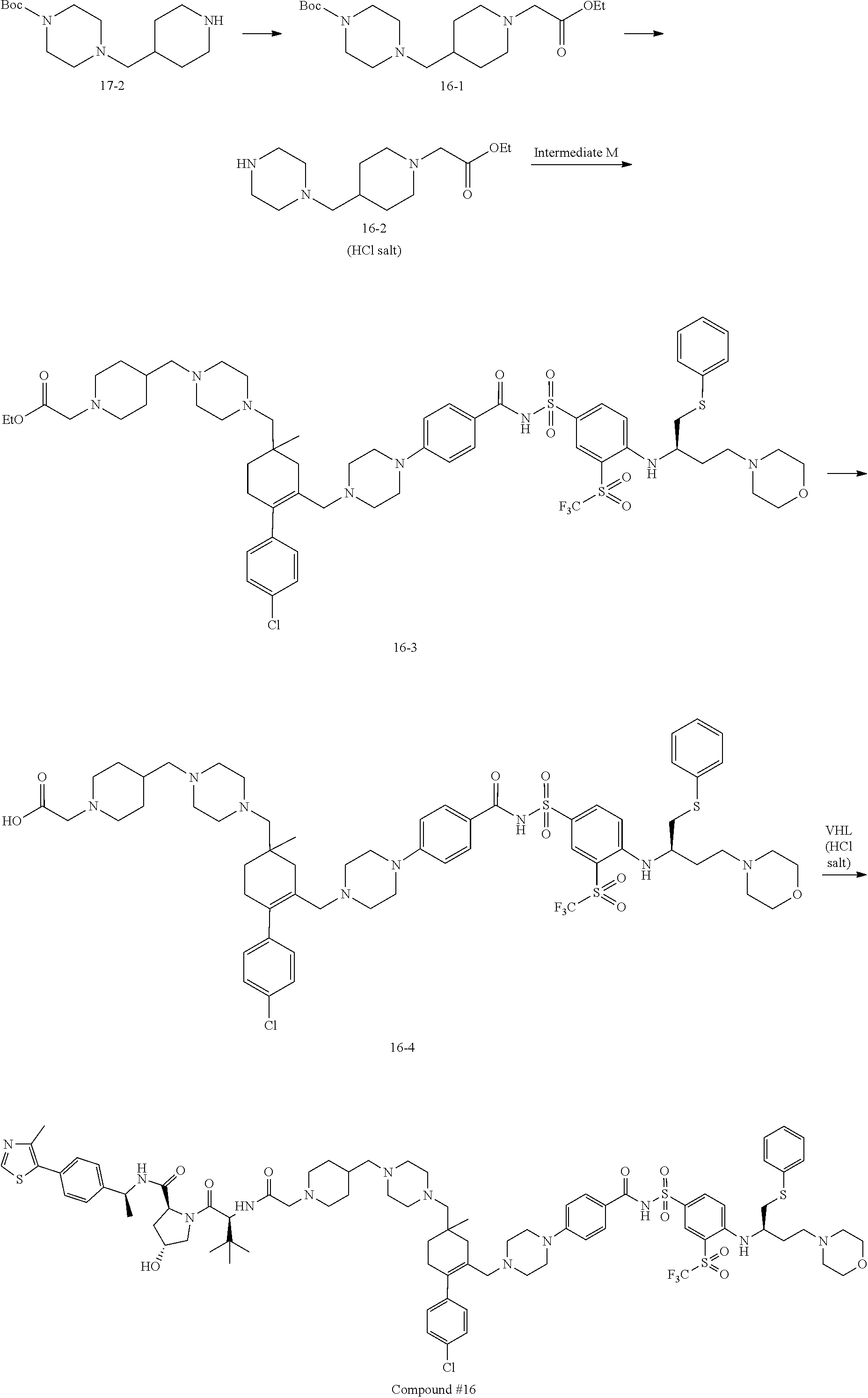

Preparation of tert-butyl 4-((1-(2-ethoxy-2-oxoethyl)piperidin-4-yl)methyl)piperazine-1-carboxylate (16-1): A mixture of ethyl bromoacetate (1.0 equiv.), 17-2 (1.05-1.2 equiv.) and DIPEA (3.0 equiv.) in DMF was stirred at 40° C. overnight. The reaction mixture was cooled to room temperature and diluted with water, then extracted with EtOAc. The combined organic layers were washed with water and sat. aq. NH₄Cl, then dried over Na₂SO₄, filtered, and concentrated. The residue was purified by flash column chromatography to afford 16−1 (396 mg, 76% yield). ¹H NMR (600 MHz, CDCl₃) δ 4.21 (q, J=7.1 Hz, 2H), 3.43 (t, J=5.1 Hz, 4H), 3.22 (s, 2H), 2.96 (dt, J=11.8, 3.3 Hz, 2H), 2.35 (t, J=5.1 Hz, 4H), 2.22-2.13 (m, 4H), 1.79-1.72 (m, 2H), 1.48 (s, 10H), 1.38-1.25 (m, 5H) ppm. LC/MS (ESI) m/z 370.3; [M+H]⁺ calcd for C₁₉H₃₆N₃O₄⁺: 370.27.

Preparation of ethyl 2-(4-(piperazin-1-ylmethyl)piperidin-1-yl)acetate hydrochloride (16-2): To a solution of 16-1 in DCM was added HCl dioxane solution (4N), then stirred at room temperature for 1 hour. The reaction mixture was concentrated to afford 16-2 as an HCl salt (16 mg, quantitative yield), which was used directly in the next step. LC/MS (ESI) m/z 270.3; [M+H]⁺ calcd for C₁₄H₂₈N₃O₂⁺: 270.22.

Preparation of ethyl 2-(4-((4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)methyl)piperidin-1-yl)acetate (16-3): A suspension of 16-2 (1.2 equiv., HCl salt), TEA (10.0 equiv.), Intermediate M (1.0 equiv.) and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by flash column chromatography to afford 16−3 (17 mg, 39% yield). ¹H NMR (600 MHz, CDCl₃) δ 8.34 (d, J=2.2 Hz, 1H), 8.01 (dd, J=9.1, 2.2 Hz, 1H), 7.88 (d, J=8.7 Hz, 2H), 7.41-7.36 (m, 2H), 7.34-7.23 (m, 5H), 7.02-6.97 (m, 2H), 6.84 (d, J=8.5 Hz, 1H), 6.75 (d, J=8.6 Hz, 2H), 6.51 (d, J=9.3 Hz, 1H), 4.19 (q, J=7.1 Hz, 2H), 3.84 (dt, J=8.4, 4.1 Hz, 1H), 3.66 (qp, J=6.2, 3.4 Hz, 5H), 3.21 (s, 3H), 3.10 (dd, J=13.8, 4.8 Hz, 1H), 3.04-2.93 (m, 3H), 2.84 (s, 2H), 2.60 (d, J=45.4 Hz, 8H), 2.49-2.18 (m, 22H), 2.17-2.10 (m, 1H), 2.07-1.97 (m, 1H), 1.77-1.61 (m, 4H), 1.55 (s, 1H), 1.51-1.46 (m, 1H), 1.43-1.36 (m, 1H), 1.28 (t, J=7.1 Hz, 3H), 1.00 (s, 3H) ppm. LC/MS (ESI) m/z 1241.4; [M+H]⁺ calcd for C₆₁H₈₁ClF₃N₈O₈S₃⁺: 1241.50.

Preparation of 2-(4-((4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)methyl)piperidin-1-yl)acetic acid (16-4): To a solution of methyl 16-3 (1.0 equiv.) in MeOH/THF was added a solution of LiOH·H₂O (5 equiv.) in water, then stirred at 40° C. overnight. The reaction mixture was concentrated to remove the organic solvents, then adjusted to pH=5-6 with 10% aq. citric acid and extracted with EtOAc. The combined organic layers were washed with brine, dried over Na₂SO₄, filtered, and concentrated to afford 16-4 (8 mg, 82% yield), which was used directly in the next step. LC/MS (ESI) m/z 1213.5; [M+H]⁺ calcd for C₅₉H₇₇ClF₃N₈O₈S₃⁺: 1213.47.

Preparation of (2S,4R)-1-((2S)-2-(2-(4-((4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)methyl)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #16): A solution of VHL-L (1.0 equiv.), 16-4 (1.0 equiv., from step 4), HATU (1.5 equiv.) and DMAP (5.0 equiv.) in DCM was stirred at room temperature overnight, then washed with sat. aq. NH₄Cl. The organic layer was dried over Na₂SO₄, filtered, and concentrated. The residue was purified by preparative TLC (DCM/MeOH) to afford compound #16 (5.7 mg, 53% yield). ¹H NMR (600 MHz, CDCl₃) δ 8.67 (d, J=3.7 Hz, 1H), 8.30 (s, 1H), 8.00 (s, 1H), 7.92−7.71 (m, 2H), 7.44-7.20 (m, 11H), 7.06-6.99 (m, 2H), 6.90 (s, OH), 6.76 (s, 2H), 6.59 (s, 1H), 5.10-5.00 (m, 1H), 4.71 (s, 1H), 4.48 (s, 1H), 4.37 (s, 1H), 4.15 (s, 1H), 3.87 (s, 1H), 3.66 (s, 6H), 3.58 (d, J=10.9 Hz, 1H), 3.32-3.06 (m, 6H), 3.06-2.90 (m, 3H), 2.84-2.56 (m, 8H), 2.52 (s, 3H), 2.47-2.22 (m, 20H), 2.10 (s, 3H), 2.05-1.95 (m, 1H), 1.91-1.80 (m, 1H), 1.74-1.36 (m, 12H), 1.12-0.93 (m, 12H) ppm. LC/MS (ESI) m/z 1639.7; [M+H]⁺ calcd for Cs₂H₁₀₇ClF₃N₁₂O₁₀S₄⁺: 1639.68.

### Example 7: Preparation of Compounds #17-19

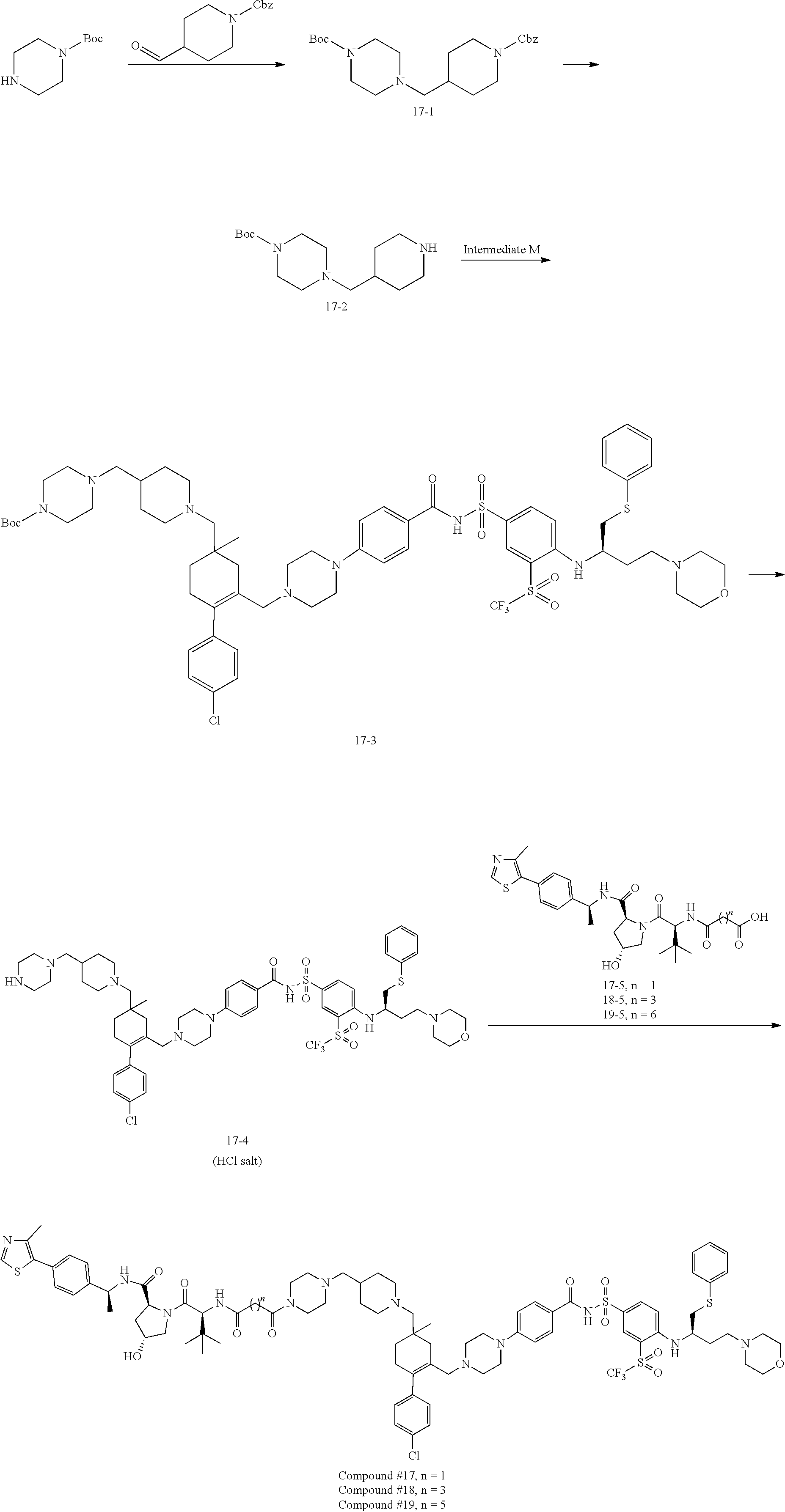

Preparation of tert-butyl 4-((1-((benzyloxy)carbonyl)piperidin-4-yl)methyl)piperazine-1-carboxylate (17-1): A suspension of benzyl 4-formylpiperidine-1-carboxylate (500 mg, 2.02 mmol), tert-butyl piperazine-1-carboxylate (415 mg, 2.23 mmol), and NaBH(OAc)₃ (645 mg, 3.04 mmol) in DCM (20 mL) was stirred at room temperature overnight. The reaction mixture was washed with sat. aq. NH₄Cl, dried over Na₂SO₄, filtered, and concentrated. The residue was purified by flash column chromatography (0% to 50% of EtOAc in hexanes) to afford 17−1 (726 mg, 86% yield). ¹H NMR (600 MHz, CDCl₃) δ 7.41-7.36 (m, 4H), 7.35-7.31 (m, 1H), 5.15 (s, 2H), 4.20 (s, 2H), 3.42 (dd, J=6.7, 3.5 Hz, 4H), 2.79 (s, 2H), 2.36 (t, J=5.0 Hz, 4H), 2.19 (d, J=7.2 Hz, 2H), 1.77 (d, J=12.9 Hz, 2H), 1.72-1.63 (m, 1H), 1.48 (s, 9H), 1.12 (s, 2H) ppm. LC/MS (ESI) m/z 418.3; [M+H]⁺ calcd for C₂₃H₃₆N₃O₄⁺: 418.27.

Preparation of tert-butyl 4-(piperidin-4-ylmethyl)piperazine-1-carboxylate (17-2): A stirring mixture of 17-1 (150 mg, 0.36 mmol) and 10% Pd(OH)₂ (15 mg) in EtOH (5 mL) was hydrogenated with a hydrogen balloon overnight. The reaction mixture was filtered through celite, the filtrate was concentrated to afford crude 17-2 (103 mg, quantitative yield), which was used directly in the next step. LC/MS (ESI) m/z 284.3; [M+H]⁺ calcd for C₁₅H₃₀N₃O₂⁺: 284.23.

Preparation of tert-butyl 4-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)methyl)piperazine-1-carboxylate (17-3): A suspension of Intermediate M (50 mg, 0.05 mmol), 17-2 (28.8 mg, 0.1 mmol), TEA (35 μL, 0.25 mmol), and NaBH(OAc)₃ (21.3 mg, 0.1 mmol) in DCM (2 mL) was stirred at room temperature overnight. The reaction mixture was washed with sat. aq. NH₄Cl, dried over Na₂SO₄, filtered, and concentrated. The residue was purified by flash column chromatography (0% to 10% of MeOH in DCM) to afford 17−3 (41 mg, 63% yield). ¹H NMR (600 MHz, CDCl₃) δ 8.33 (d, J=2.2 Hz, 1H), 8.01 (dd, J=9.1, 2.2 Hz, 1H), 7.85 (d, J=8.5 Hz, 2H), 7.40-7.35 (m, 2H), 7.30 (dd, J=7.8, 4.6 Hz, 4H), 7.27-7.21 (m, 1H), 7.02 (d, J=8.1 Hz, 2H), 6.84 (d, J=8.6 Hz, 1H), 6.66 (d, J=8.5 Hz, 2H), 6.55 (d, J=9.3 Hz, 1H), 3.85 (dt, J=8.6, 4.4 Hz, 1H), 3.66 (tp, J=9.0, 4.4 Hz, 4H), 3.45-3.06 (m, 11H), 2.98 (dd, J=13.7, 7.5 Hz, 2H), 2.84-2.48 (m, 7H), 2.47-2.26 (m, 16H), 2.19 (d, J=5.4 Hz, 2H), 2.12 (dtd, J=14.8, 7.4, 4.0 Hz, 1H), 2.05-1.89 (m, 1H), 1.80 (s, 2H), 1.71-1.58 (m, 3H), 1.55-1.50 (m, 1H), 1.45 (s, 9H), 1.10 (s, 3H) ppm. LC/MS (ESI) m/z 1255.5; [M+H]⁺ calcd for C₆₂H₈₃ClF₃N₈O₈S₃⁺: 1255.51.

Preparation of 4-(4-((4′-chloro-4-methyl-4-((4-(piperazin-1-ylmethyl)piperidin-1-yl)methyl)-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (17-4): To a solution of 17-3 (35 mg, 0.028 mmol) in DCM (1 mL) was added 4N HCl in dioxane (1 mL), then stirred at room temperature for 2 hours. The reaction mixture was concentrated to afford crude 17-4 (9.3 mg) as an HCl salt, which was used directly in the next step. LC/MS (ESI) m/z 1155.3; [M+H]+ calcd for C₅₇H₇₅ClF₃N₈O₆S₃⁺: 1155.46.

General Procedure H was used for the last step preparation of compounds #17-19. (2S,4R)-1-((2S)-2-(3-(4-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)methyl)piperazin-1-yl)-3-oxopropanamido)-3,3-dimethylbutanoyl)-4-hydroxy-N—((S)-1-(4-(4-methylthiazol-5-yl)phenyl)ethyl)pyrrolidine-2-carboxamide (compound #17) (5.4 mg, 43% yield). ¹H NMR (600 MHz, CDCl₃) δ 8.68 (s, 1H), 8.33 (d, J=2.3 Hz, 1H), 8.04 (d, J=9.0 Hz, 1H), 7.82 (d, J=8.1 Hz, 2H), 7.60 (d, J=7.9 Hz, 1H), 7.43-7.34 (m, 6H), 7.33-7.21 (m, 5H), 7.00 (d, J=8.0 32 Hz, 2H), 6.72 (d, J=8.6 Hz, 2H), 6.56 (d, J=9.2 Hz, 1H), 5.08 (p, J=6.9 Hz, 1H), 4.73 (t, J=8.1 Hz, 1H), 4.52 (d, J=5.0 Hz, 1H), 4.48 (s, 1H), 4.07 (d, J=11.4 Hz, 1H), 3.86 (s, 1H), 3.70-3.50 (m, 8H), 3.47-3.37 (m, 2H), 3.32 (d, J=10.8 Hz, 1H), 3.27-3.07 (m, 5H), 3.00 (dd, J=13.9, 7.3 Hz, 1H), 2.92 (s, 1H), 2.43 (d, J=117.5 Hz, 32H), 2.22-2.05 (m, 4H), 1.73-1.40 (m, 8H), 1.06 (d, J=17.7 Hz, 12H) ppm. LC/MS (ESI) m/z 1667.4; [M+H]⁺ calcd for Cs₃H₁₀₇ClF₃N₁₂O₁₁S₄⁺: 1667.67.

(2S,4R)-1-((2S)-2-(5-(4-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)methyl)piperazin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (compound #18) (4.7 mg, 37% yield). ¹H NMR (600 MHz, CDCl₃) δ 8.68 (s, 1H), 8.33 (d, J=2.2 Hz, 1H), 8.04 (d, J=9.1 Hz, 1H), 7.82 (d, J=8.4 Hz, 2H), 7.54 (s, 1H), 7.43-7.33 (m, 7H), 7.32-7.21 (m, 4H), 6.99 (d, J=8.2 Hz, 2H), 6.74 (d, J=8.6 Hz, 2H), 6.56 (d, J=9.3 Hz, 1H), 5.08 (p, J=6.9 Hz, 1H), 4.71 (t, J=8.2 Hz, 1H), 4.55 (q, J=4.3 Hz, 1H), 4.48 (s, 1H), 4.09 (d, J=11.4 Hz, 1H), 3.85 (s, 1H), 3.71-3.56 (m, 6H), 3.48 (s, 1H), 3.36 (s, 2H), 3.18 (s, 4H), 3.12-2.94 (m, 3H), 2.85 (s, 2H), 2.53 (d, J=4.9 Hz, 3H), 2.49-2.22 (m, 30H), 2.22-2.07 (m, 4H), 2.00-1.86 (m, 4H), 1.76-1.56 (m, 4H), 1.51-1.42 (m, 3H), 1.36 (s, 1H), 1.03 (d, J=25.0 Hz, 12H) ppm. LC/MS (ESI) m/z 1695.8; [M+H]⁺ calcd for C₈₅H₁₁₁ClF₃N₁₂O₁₁S₄⁺: 1695.70.

(2S,4R)-1-((2S)-2-(7-(4-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)methyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (compound #19) (6.1 mg, 47% yield). ¹H NMR (600 MHz, CDCl₃) δ 8.68 (d, J=2.5 Hz, 1H), 8.32 (d, J=2.2 Hz, 1H), 8.04 (dd, J=9.2, 2.2 Hz, 1H), 7.82 (d, J=8.5 Hz, 2H), 7.49-7.33 (m, 8H), 7.32-7.21 (m, 5H), 7.00-6.97 (m, 2H), 6.73 (d, J=8.5 Hz, 2H), 6.57 (dd, J=15.5, 7.6 Hz, 2H), 5.08 (p, J=6.6 Hz, 1H), 4.68 (dt, J=19.6, 8.2 Hz, 1H), 4.62-4.58 (m, 1H), 4.49 (s, 1H), 4.05 (dd, J=21.7, 11.4 Hz, 1H), 3.86 (s, 1H), 3.71-3.56 (m, 6H), 3.55-3.32 (m, 3H), 3.18 (s, 4H), 3.13-2.94 (m, 3H), 2.85 (s, 2H), 2.53 (d, J=3.8 Hz, 3H), 2.50-2.07 (m, 34H), 1.74-1.56 (m, 6H), 1.54-1.42 (m, 5H), 1.42-1.29 (m, 3H), 1.08-0.97 (m, 12H) ppm. LC/MS (ESI) m/z 1723.7; [M+H]⁺ calcd for Cs₇H₁₁₅ClF₃N₁₂O₁₁S₄⁺: 1723.73.

### Example 8: Preparation of Compound #20

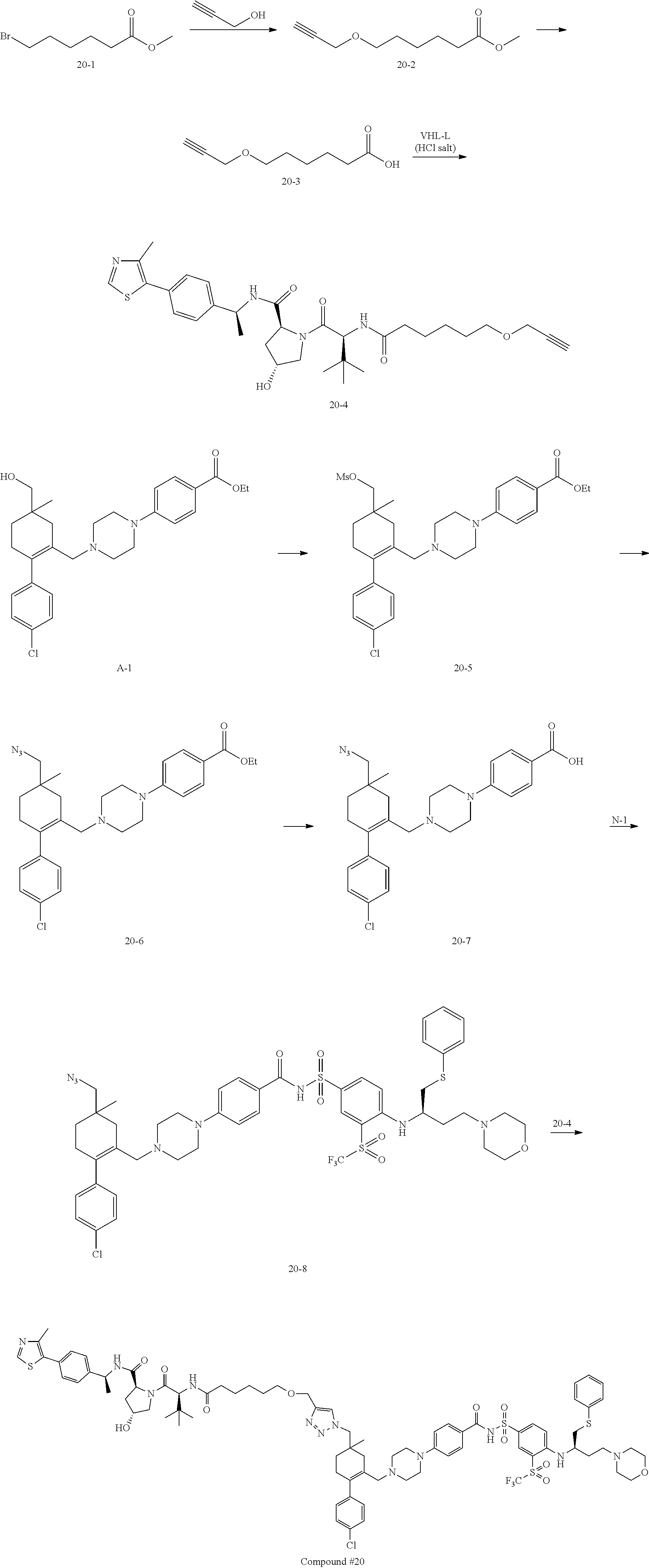

Preparation of methyl 6-(prop-2-yn-1-yloxy)hexanoate (20-2): Under an argon atmosphere, to a suspension of 60% of NaH (250 mg, 6.22 mmol) in anhydrous DMF (5 mL) was added 2-propyn-1-ol (565 μL, 9.57 mmol) dropwise at 0-5° C. with stirring. After stirring at 0-5° C. for an extra 20 min, 20-1 (1 g, 4.78 mmol) was added to above mixture. Then the resulting mixture was warmed up to 50° C. and stirred overnight. The mixture was diluted with EtOAc and washed with water and brine, dried over Na₂SO₄, filtered, and concentrated. The residue was purified by flash column chromatography (0% to 20% of EtOAc in hexanes) to afford 20-2 (655 mg, 74% yield). LC/MS (ESI) m/z 185.1; [M+H]⁺ calcd for C₁₀H₁₇O₃⁺: 185.12.

Preparation of 6-(prop-2-yn-1-yloxy)hexanoic acid (20-3): To a solution of 20-2 (300 mg, 1.63 mmol) in MeOH/THF (1.5 mL/0.3 mL) was added 1 M aq. NaOH (0.5 mL), then stirred at room temperature overnight. The reaction mixture was concentrated to remove the organic solvents, then adjusted to pH=4-5 with 10% aq. citric acid and extracted with Et₂O. The combined organic layers were washed with brine, dried over Na₂SO₄, filtered, and concentrated to afford crude 20-3 (289 mg, quantitative yield), which was used directly in the next step. LC/MS (ESI) m/z 171.1; [M+H]⁺ calcd for C₉H₁₅O₃⁺: 171.10.

Preparation of (2S,4R)-1-((S)-3,3-dimethyl-2-(6-(prop-2-yn-1-yloxy)hexanamido)butanoyl)-4-hydroxy-N—((S)-1-(4-(4-methylthiazol-5-yl)phenyl)ethyl)pyrrolidine-2-carboxamide (20-4): A mixture of 20−3 (35 mg, 0.21 mmol), HATU (100 mg, 0.26 mmol), VHL-L (100 mg, 0.2 mmol, HCl salt), and TEA (150 μL, 2.0 mmol) in DCM (2 mL) was stirred at room temperature for 3 hours. The reaction mixture was washed with sat. aq. NH₄Cl. The organic layers were dried over Na₂SO₄, filtered, and concentrated. The residue was purified by flash column chromatography (0% to 5% of MeOH in DCM) to afford 20−4 (99 mg, 81% yield). ¹H NMR (600 8 MHz, CDCl₃) δ 8.69 (s, 1H), 7.46 (d, J=7.9 Hz, 1H), 7.44-7.36 (m, 4H), 6.28 (d, J=8.8 Hz, 1H), 5.10 (p, J=7.1 Hz, 1H), 4.69 (t, J=7.9 Hz, 1H), 4.58 (d, J=8.8 Hz, 1H), 4.54-4.49 (m, 1H), 4.12 (d, J=2.4 Hz, 2H), 4.07 (dt, J=11.4, 1.9 Hz, 1H), 3.88 (s, 1H), 3.63 (dd, J=11.3, 3.7 Hz, 1H), 3.50 (t, J=6.4 Hz, 2H), 2.54 (s, 3H), 2.48-2.41 (m, 2H), 2.19 (td, J=7.4, 2.5 Hz, 2H), 2.12-2.05 (m, 1H), 1.66-1.56 (m, 4H), 1.49 (d, J=6.9 Hz, 3H), 1.43-1.33 (m, 2H), 1.05 (s, 9H) ppm. LC/MS (ESI) m/z 597.3; [M+H]⁺ calcd for C₃₂H₄₅N₄O₅S⁺: 597.31.

Preparation of ethyl 4-(4-((4′-chloro-4-methyl-4-(((methylsulfonyl)oxy)methyl)-3,4,5,6-tetrahydro-[1,1′-biphenyl]-2-yl)methyl)piperazin-1-yl)benzoate (20-5): Under an argon atmosphere, to a stirring solution of A-1 (260 mg, 0.54 mmol) and TEA (225 μL, 1.62 mmol) in DCM (3 mL) was added dropwise methane sulfonyl chloride (50 μL, 0.65 mmol) at 0° C. After the addition completed, the resulting mixture was warmed up to room temperature and stirred for hour. The reaction mixture was concentrated and purified by flash column chromatography (0% to 50% of EtOAc in hexanes) to afford 20-5 (270 mg, 89% yield). ¹H NMR (600 MHz, CDCl₃) δ 7.95-7.89 (m, 2H), 7.34-7.29 (m, 2H), 7.04-6.99 (m, 2H), 6.86-6.81 (m, 2H), 4.07 (q, J=9.2 Hz, 2H), 3.28 (t, J=4.2 Hz, 4H), 3.06 (s, 3H), 2.83 (s, 2H), 2.37 (t, J=5.1 Hz, 4H), 2.35-2.30 (m, 2H), 2.23 (dt, J=17.8, 2.3 Hz, 1H), 2.11 (dd, J=18.1, 1.9 Hz, 1H), 1.71 (dt, J=13.6, 6.9 Hz, 1H), 1.64-1.56 (m, 3H), 1.38 (t, J=7.1 Hz, 3H), 1.12 (s, 3H) ppm. LC/MS (ESI) m/z 561.2; [M+H]⁺ calcd for C₂₉H₃₈ClN₂O₅S⁺: 561.22.

Preparation of ethyl 4-(4-((4-(azidomethyl)-4′-chloro-4-methyl-3,4,5,6-tetrahydro-[1,1′-biphenyl]-2-yl)methyl)piperazin-1-yl)benzoate (20−6): A mixture of 20-5 (270 mg, 0.48 mmol) and NaN₃ (156 mg, 2.4 mmol) in DMF (3 mL) was heated at 120° C. overnight. The reaction mixture was cooled to room temperature and diluted with EtOAc, washed with water and brine. The organic layers were dried over Na₂SO₄, filtered, and concentrated. The residue was purified by flash column chromatography (0% to 25% of EtOAc in hexanes) to afford 20-6 (165 mg, 68% yield). ¹H NMR (600 MHz, CDCl₃) δ 7.94-7.89 (m, 2H), 7.33-7.29 (m, 2H), 7.04-6.99 (m, 2H), 6.86-6.82 (m, 2H), 4.34 (q, J=7.1 Hz, 2H), 3.31-3.23 (m, 6H), 2.83 (s, 2H), 2.37 (t, J=5.1 Hz, 4H), 2.35-2.24 (m, 1H), 2.22-2.14 (m, 1H), 2.09-2.02 (m, 1H), 1.68-1.61 (m, 1H), 1.57-1.50 (m, 1H), 1.38 (t, J=7.1 Hz, 3H), 1.06 (s, 3H) ppm. LC/MS (ESI) m/z 508.1; [M+H]+ calcd for C₂₈H₃₅ClN₅O₂⁺: 508.25.

Preparation of 4-(4-((4-(azidomethyl)-4′-chloro-4-methyl-3,4,5,6-tetrahydro-[1,1′-biphenyl]-2-yl)methyl)piperazin-1-yl)benzoic acid (20-7): To a solution of 20-6 (50 mg, 0.098 mmol) in THF/MeOH (0.5 mL/0.1 mL) was added a solution of LiOH·H₂O (15 mg, 1.19 mmol) in water (0.1 mL), then stirred at 45° C. overnight. The reaction mixture was concentrated to remove the organic solvents, then adjusted to pH=5-6 with 5% aq. citric acid and extracted with EtOAc. The combined organic layers were washed with brine, dried over Na₂SO₄, filtered, and concentrated to afford 20-7 (47 mg, quantitative yield). LC/MS (ESI) m/z 480.2; [M+H]⁺ calcd for C₂₆H₃₁ClN₅O₂⁺: 480.22.

Preparation of 4-(4-((4-(azidomethyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (20-8): A mixture of 20-7 (47 mg, 0.097 mmol), N-1 (49 mg, 0.088 mmol), EDC (85 mg, 0.44 mmol), and DMAP (54 mg, 0.44 mmol) in DCM (2 mL) was stirred at room temperature overnight. The reaction mixture was washed with water and sat. aq. NH₄Cl, dried over Na₂SO₄, filtered, and concentrated. The residue was purified by flash column chromatography (0% to 5% of MeOH in DCM) to afford 20−8 (56 mg, 62% yield). LC/MS (ESI) m/z 1015.2; [M+H]⁺ calcd for C₄₇H₅₅ClF₃N₈₆S₃⁺: 1015.30.

Preparation of (2S,4R)-1-((2S)-2-(6-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1H-1,2,3-triazol-4-yl)methoxy)hexanamido)-3,3-dimethylbutanoyl)-4-hydroxy-N—((S)-1-(4-(4-methylthiazol-5-yl)phenyl)ethyl)pyrrolidine-2-carboxamide (compound #20): General Procedure B was applied to obtain compound #20 (17.6 mg, 74% yield). ¹H NMR (600 MHz, CDCl₃) δ 8.68 (s, 1H), 8.32 (d, J=2.2 Hz, 1H), 8.09 (dt, J=9.3, 1.6 Hz, 1H), 7.79-7.71 (m, 3H), 7.47 (t, J=7.8 Hz, 1H), 7.43-7.35 (m, 6H), 7.30 (dd, J=8.2, 6.6 Hz, 4H), 7.27-7.23 (m, 1H), 7.04-6.97 (m, 3H), 6.76 (d, J=8.7 Hz, 2H), 6.62 (d, J=9.4 Hz, 1H), 6.52 (dd, J=9.1, 3.3 Hz, 1H), 5.10 (p, J=7.1 Hz, 1H), 4.70 (t, J=8.2 Hz, 1H), 4.68-4.63 (m, 1H), 4.59 (s, 2H), 4.50 (d, J=4.1 Hz, 1H), 4.34-4.26 (m, 2H), 4.11 (d, J=11.5 Hz, 1H), 3.90 (dp, J=8.6, 4.1 Hz, 1H), 3.72-3.62 (m, 4H), 3.60 (dd, J=11.4, 3.4 Hz, 1H), 3.40 (t, J=6.7 Hz, 2H), 3.22 (t, J=5.2 Hz, 4H), 3.11 (dd, J=13.8, 5.1 Hz, 1H), 3.03 (dd, J=13.9, 7.2 Hz, 1H), 2.88 (d, 1H), 2.82 (d, J=12.6 Hz, 1H), 2.52 (s, 3H), 2.49-2.23 (m, 16H), 2.18-2.06 (m, 4H), 2.02 (d, J=17.4 Hz, 1H), 1.73-1.58 (m, 3H), 1.53-1.37 (m, 6H), 1.20 (t, J=7.2 Hz, 2H), 1.05 (s, 9H), 0.99 (d, J=2.8 Hz, 3H) ppm. LC/MS (ESI) m/z 1611.7; [M+H]⁺ calcd for C₇₉H₉₉ClF₃N₁₂O₁₁S₄⁺: 1611.61.

### Example 9: Preparation of Compound #21

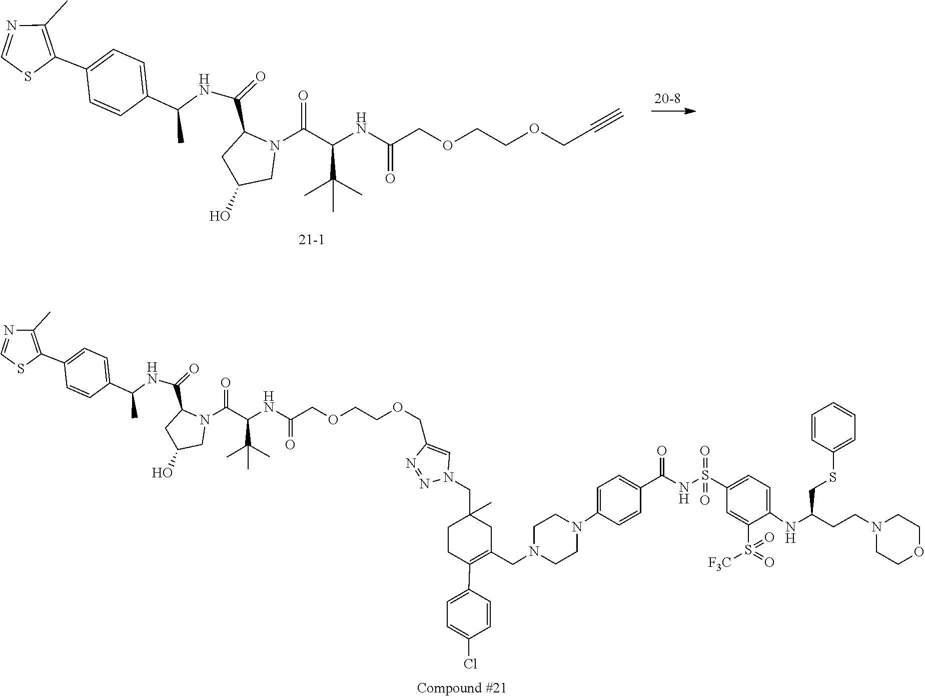 [SMILES: C#CCOCCOCC(=O)N[C@H](C(=O)N1C[C@H](O)C[C@H]1C(=O)N[C@@H](C)c1ccc(-c2scnc2C)cc1)C(C)(C)C ; Cc1ncsc1-c1ccc([C@H](C)NC(=O)[C@@H]2C[C@@H](O)CN2C(=O)[C@@H](NC(=O)COCCOCc2cn(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1]

Compound #21 was prepared by following General Procedure B. (2S,4R)-1-((2S)-2-(2-(2-((1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1H-1,2,3-triazol-4-yl)methoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (compound #21) (12.5 mg, 57% yield). ¹H NMR (600 MHz, CDCl₃) δ 8.70 (s, 1H), 8.34 (d, J=2.3 Hz, 1H), 8.11 (d, J=9.2 Hz, 1H), 7.78 (d, J=7.7 Hz, 1H), 7.72 (d, J=8.4 Hz, 2H), 7.49 (s, 1H), 7.41−7.35 (m, 7H), 7.34-7.22 (m, 5H), 7.05-6.98 (m, 2H), 6.76 (dd, J=9.1, 2.9 Hz, 2H), 6.61 (d, J=9.4 Hz, 1H), 5.13-5.05 (m, 1H), 4.75-4.67 (m, 3H), 4.59-4.48 (m, 2H), 4.30 (s, 2H), 4.12 (d, J=11.4 Hz, 1H), 3.90 (d, J=8.1 Hz, 1H), 3.86-3.71 (m, 2H), 3.71-3.58 (m, 7H), 3.53 (s, 2H), 3.21 (d, J=7.4 Hz, 4H), 3.11 (dd, J=13.9, 5.0 Hz, 1H), 3.02 (dd, J=13.9, 7.2 Hz, 1H), 2.88 (d, J=12.6 Hz, 1H), 2.82 (d, J=12.8 Hz, 1H), 2.51 (s, 3H), 2.48-2.23 (m, 16H), 2.16-2.07 (m, 2H), 2.04 (d, J=17.5 Hz, 1H), 1.72-1.65 (m, 1H), 1.62 (t, J=6.7 Hz, 2H), 1.49 (dd, J=6.9, 2.7 Hz, 3H), 1.06 (s, 9H), 0.98 (s, 3H) ppm. LC/MS (ESI) m/z 1599.6; [M+H]⁺ calcd for C₇₇H₉₅ClF₃N₁₂O₁₂S₄⁺: 1599.57.

### Example 10: Preparation of Compound #22

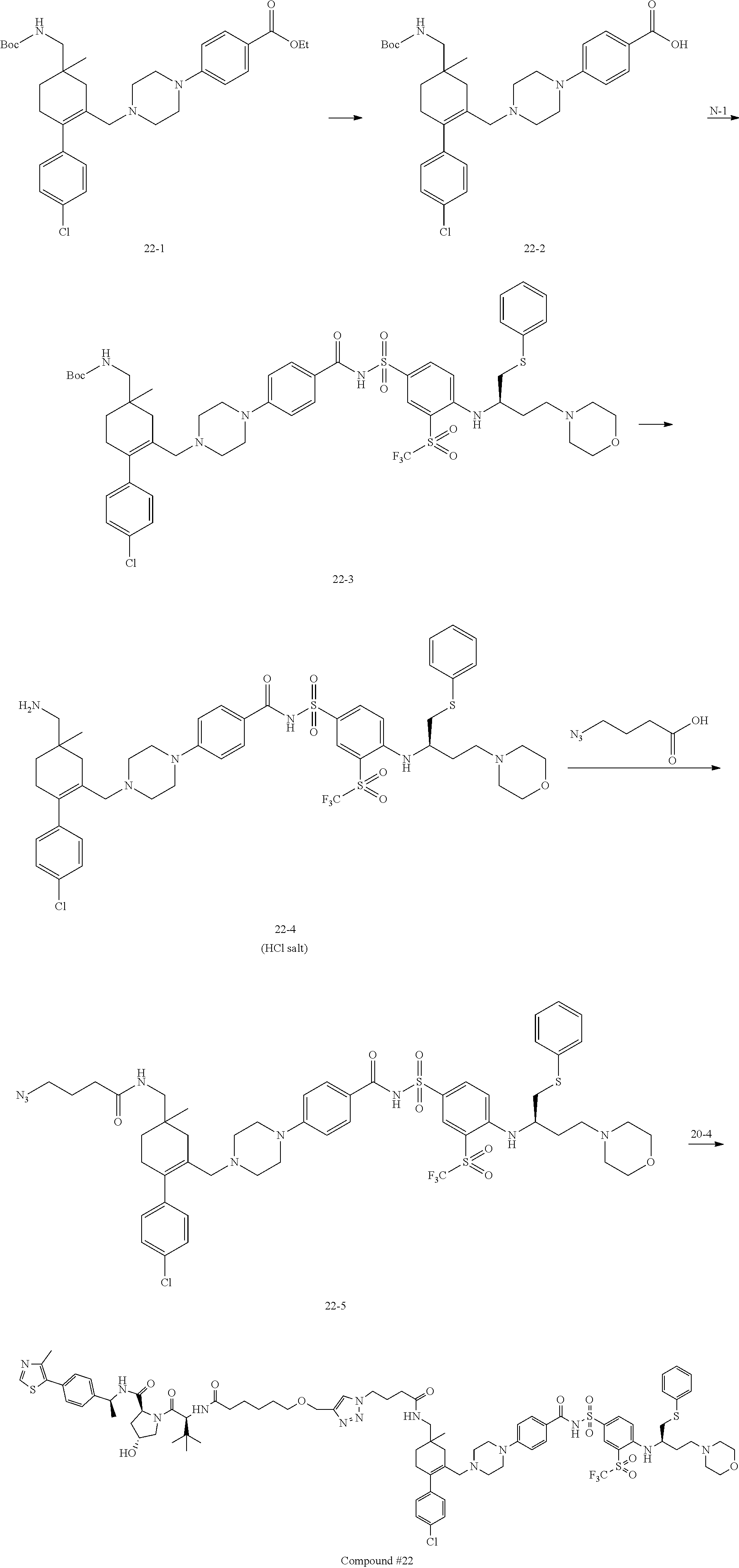

Preparation of 4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoic acid (22-2): To a solution of 22-1 (39 mg, 0.067 mmol) in THF/MeOH (0.3 mL/0.1 mL) was added a solution of LiOH·H₂O (15 mg, 0.36 mmol) in water (0.1 mL), then stirred at 45° C. overnight. The reaction mixture was concentrated to remove the organic solvents, then adjusted to pH=5-6 with 10% aq. citric acid and extracted with EtOAc. The combined organic layers were washed with brine, dried over Na₂SO₄, filtered, and concentrated to afford crude product 22-2 (37 mg, quantitative yield), which was used directly in the next step. LC/MS (ESI) m/z 554.2; [M+H]⁺ calcd for C₃₁H₄₁ClN₃O₄⁺: 554.28.

Preparation of tert-butyl ((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)carbamate (22-3): A mixture of 22-2 (37 mg, 0.067 mmol), N-1 (34 mg, 0.061 mmol), EDC (60 mg, 0.31 mmol), and DMAP (38 mg, 0.31 mmol) in DCM (1.5 mL) was stirred at room temperature overnight. The reaction mixture was washed with water and sat. aq. NH₄Cl, dried over Na₂SO₄, filtered, and concentrated. The residue was purified by flash column chromatography (0% to 5% of MeOH in DCM) to afford 22−3 (27 mg, 40% yield). ¹H NMR (600 MHz, CDCl₃) δ 8.36 (d, J=2.3 Hz, 1H), 8.14-8.07 (m, 1H), 7.70 (d, J=8.3 Hz, 2H), 7.39 (d, J=7.1 Hz, 2H), 7.35-7.22 (m, 6H), 7.05-6.98 (m, 3H), 6.74 (d, J=8.6 Hz, 2H), 6.60 (d, J=9.3 Hz, 1H), 4.84 (s, 1H), 3.90 (s, 1H), 3.75-3.62 (m, 4H), 3.36-3.23 (m, 4H), 3.13 (td, J=13.7, 6.0 Hz, 2H), 3.04 (td, J=13.6, 6.6 Hz, 2H), 2.91 (s, 2H), 2.53 4-2.23 (m, 11H), 2.15 (d, J=18.0 Hz, 2H), 2.01 (d, J=17.7 Hz, 1H), 1.76-1.64 (m, 1H), 1.62-1.54 (m, 1H), 1.54-1.39 (m, 11H), 0.98 (s, 3H) ppm. LC/MS (ESI) m/z 1089.5; [M+H]⁺ calcd for C₅₂H₆₅ClF₃N₆O₈S₃⁺: 1089.37.

Preparation of 4-(4-((4-(aminomethyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (22−4): To solution of 22-3 (12 mg, 0.011 mmol) in DCM (0.2 mL) was added 4N HCl in dioxane (0.2 mL), then stirred at room temperature for 1 hour. The reaction mixture was concentrated to afford the crude product 22-4 (11 mg, quantitative yield) as an HCl salt, which was used directly in the next step. LC/MS (ESI) m/z 989.2; [M+H]⁺ calcd for C₄₇H₅₇ClF₃N₆O₆S₃⁺: 989.31.

Preparation of 4-(4-((4-((4-azidobutanamido)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (22-5): To a solution of 22-4 (12 mg, 0.011 mmol) and TEA (15 μL, 0.2 mmol) in DCM (0.2 mL) was added a solution of 4-azidobutanoic acid (1.7 mg, 0.013 mmol) and HATU (5.5 mg, 0.014 mmol) in DCM (0.2 mL), then stirred at room temperature for 3 hours. The reaction mixture was washed with sat. aq. NH₄Cl. The organic layers were dried over Na₂SO₄, filtered, and concentrated. The residue was purified by flash column chromatography (0% to 60% of EtOAc in hexanes) to afford 22−5 (10 mg, 83% yield). ¹H NMR (600 MHz, CDCl₃) δ 8.37 (d, J=2.2 Hz, 1H), 8.05 (dd, J=9.1, 2.3 Hz, 1H), 7.71 (d, J=8.4 Hz, 2H), 7.43-7.38 (m, 2H), 7.38-7.30 (m, 4H), 7.31-7.25 (m, 1H), 7.05-7.00 (m, 2H), 6.93 (d, J=8.6 Hz, 1H), 6.64 (d, J=9.3 Hz, 1H), 6.54 (d, J=8.5 Hz, 2H), 3.91 (s, 1H), 3.73-3.63 (m, 5H), 3.56 (s, 2H), 3.36-3.23 (m, 4H), 3.21-3.16 (m, 2H), 3.13 (dd, J=13.7, 5.0 Hz, 1H), 3.03 (dd, J=13.8, 7.3 Hz, 1H), 2.95 (d, J=13.1 Hz, 1H), 2.50-2.26 (m, 16H), 2.14 (s, 1H), 1.87 (p, J=7.1 Hz, 2H), 1.74-1.64 (m, 1H), 1.60-1.53 (m, 2H), 1.03 (s, 3H) ppm. LC/MS (ESI) m/z 1100.4; [M+H]⁺ calcd for C₅₁H₆₂ClF₃N₉O₇S₃⁺: 1100.36.

Preparation of (2S,4R)-1-((2S)-2-(6-((1-(4-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)amino)-4-oxobutyl)-1H-1,2,3-triazol-4-yl)methoxy)hexanamido)-3,3-dimethylbutanoyl)-4-hydroxy-N—((S)-1-(4-(4-methylthiazol-5-yl)phenyl)ethyl)pyrrolidine-2-carboxamide (compound #22): General Procedure B was performed to form compound #22 (8.6 mg, 56% yield). ¹H NMR (600 MHz, CDCl₃) δ 8.68 (s, 1H), 8.33 (d, J=2.2 Hz, 1H), 8.08-8.02 (m, 1H), 7.73 (d, J=8.4 Hz, 2H), 7.55 (s, 1H), 7.49 (d, J=7.6 Hz, 1H), 7.43-7.35 (m, 6H), 7.34-7.22 (m, 5H), 7.04-6.99 (m, 2H), 6.95 (d, J=8.5 Hz, 1H), 6.63 (t, J=8.8 Hz, 3H), 6.52 (dd, J=9.0, 4.3 Hz, 1H), 5.09 (p, J=7.1 Hz, 1H), 4.67 (t, 1H), 4.59 (dd, J=8.9, 2.0 Hz, 1H), 4.52 (d, J=2.8 Hz, 2H), 4.48 (s, 1H), 4.33 (t, J=6.7 Hz, 2H), 4.06 (d, J=11.3 Hz, 1H), 3.94-3.85 (m, 1H), 3.70-3.62 (m, 4H), 3.62-3.57 (m, 1H), 3.47-3.44 (m, 2H), 3.37 (s, 1H), 3.22 (s, 5H), 3.16-2.98 (m, 3H), 2.52 (s, 3H), 2.48-2.07 (m, 22H), 1.74-1.51 (m, 10H), 1.48 (dd, J=7.0, 1.7 Hz, 3H), 1.32 (p, J=8.0 Hz, 2H), 1.04 (s, 9H), 1.01 (s, 3H) ppm. LC/MS (ESI) m/z 1696.7; [M+H]⁺ calcd for C₈₃H₁₀₆ClF₃N₁₃O₁₂S₄⁺: 1696.66.

### Example 11: Preparation of Compound #23

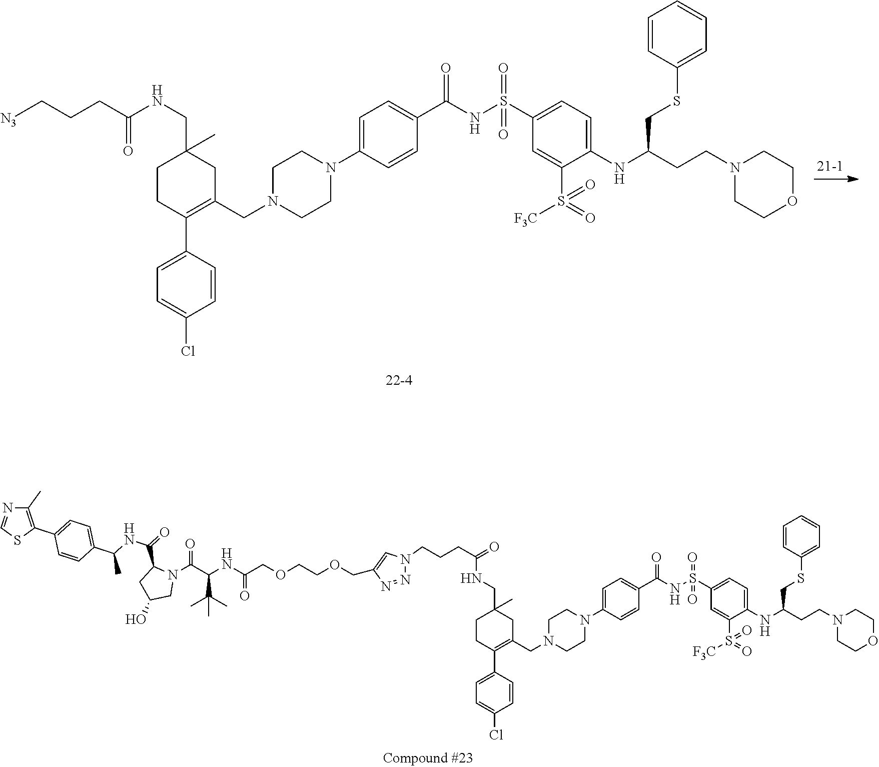 [SMILES: CC1(CNC(=O)CCCN=[N+]=[N-])CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1 ; "22-4" ; Cc1ncsc1-c1ccc([C@H](C)NC(=O)[C@@H]2C[C@@H](O)CN2C(=O)[C@@H](NC(=O)COCCOCc2cn(CCCC(=O)NCC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)nn2)C(C)(C)C)cc1]

Compound #23 was prepared by following General Procedure B. (2S,4R)-1-((2S)-2-(2-(2-((1-(4-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)amino)-4-oxobutyl)-1H-1,2,3-triazol-4-yl)methoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (compound #23) (9.8 mg, 43% yield). ¹H NMR (600 MHz, CDCl₃) δ 8.70 (s, 1H), 8.32 (d, J=2.4 Hz, 1H), 8.08 (d, J=9.1 Hz, 1H), 7.80 (d, J=7.0 Hz, 1H), 7.74 (d, J=7.9 Hz, 2H), 7.66 (d, J=7.6 Hz, 1H), 7.50 (dd, J=9.2, 6.7 Hz, 1H), 7.41-7.35 (m, 6H), 7.31 (td, J=7.9, 2.1 Hz, 4H), 7.25 (t, J=7.3 Hz, 1H), 7.04-6.99 (m, 2H), 6.93 (d, J=8.5 Hz, 1H), 6.63 (d, J=8.6 Hz, 2H), 5.09 (p, J=7.1 Hz, 1H), 4.75-4.64 (m, 2H), 4.61 (d, 2H), 4.48 (s, 1H), 4.32 (td, J=13.7, 6.5 Hz, 2H), 4.00 (dd, J=14.1, 5.0 Hz, 2H), 3.97-3.85 (m, 2H), 3.75-3.58 (m, 9H), 3.35 (s, 1H), 3.23 (s, 5H), 3.15-2.96 (m, 3H), 2.50 (s, 3H), 2.47-2.05 (m, 22H), 1.73-1.59 (m, 2H), 1.59-1.51 (m, 2H), 1.48 (dd, J=6.9, 1.9 Hz, 3H), 1.35-1.22 (m, 1H), 1.05 (s, 9H), 0.99 (d, J=6.1 Hz, 3H) ppm. LC/MS (ESI) m/z 1684.5; [M+H]⁺ calcd for C₈₁H₁₀₂ClF₃N₁₃O₁₃S₄⁺: 1684.62.

### Example 12: Preparation of Compound #25

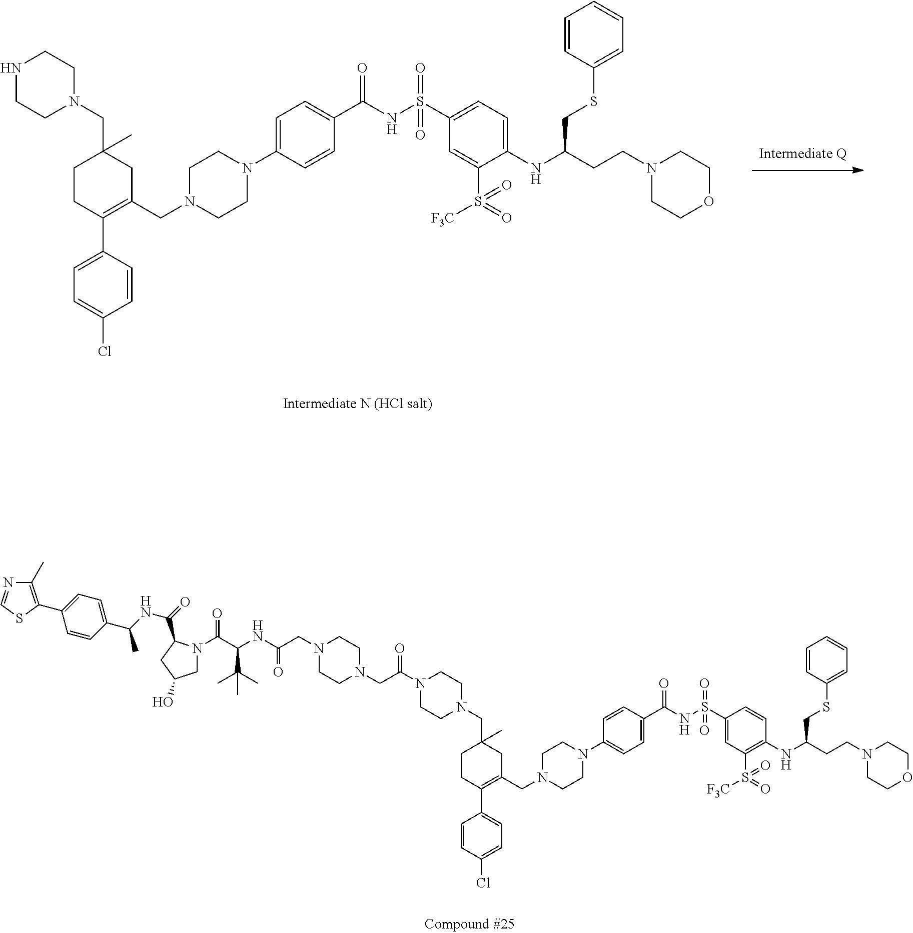 [SMILES: CC1(CN2CCNCC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CN2CCN(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1]

Compound #25 was prepared by following General Procedure H. (2S,4R)-1-((2S)-2-(2-(4-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #25). ¹H NMR (600 MHz, Chloroform-d) δ 8.67 (s, 1H), 8.33 (s, 1H), 8.09 (d, J=10.0 Hz, 1H), 7.79 (d, J=34.9 Hz, 2H), 7.38 (dt, J=18.1, 6.8 Hz, 7H), 7.34−7.27 (m, 4H), 7.01 (s, 3H), 6.70 (d, J=57.8 Hz, 2H), 5.13 16-5.05 (m, 1H), 4.75 (d, J=9.4 Hz, 1H), 4.54 (d, J=44.8 Hz, 2H), 4.17 (s, 1H), 3.94 (s, 1H), 3.84-3.51 (m, 7H), 3.50-3.15 (m, 2H), 3.14-2.97 (m, 4H), 2.88 (s, 2H), 2.64 (d, J=15.9 Hz, 5H), 2.55-2.44 (m, 13H), 2.32 (s, 10H), 2.20-1.86 (m, 4H), 1.73 (s, 1H), 1.47 (d, J=7.0 Hz, 3H), 1.41 (t, J=7.3 Hz, 3H), 1.25 (s, 5H), 1.05 (d, J=5.2 Hz, 9H), 0.97 (s, 4H), 0.88 (d, J=6.5 Hz, 2H) ppm. LC/MS (ESI) m/z 1668.9; [M+H]⁺ calcd for C₈₂H₁₀₆ClF₃N₁₃O₁₁S₄⁺: 1668.67.

### Example 13: Preparation of Compound #26

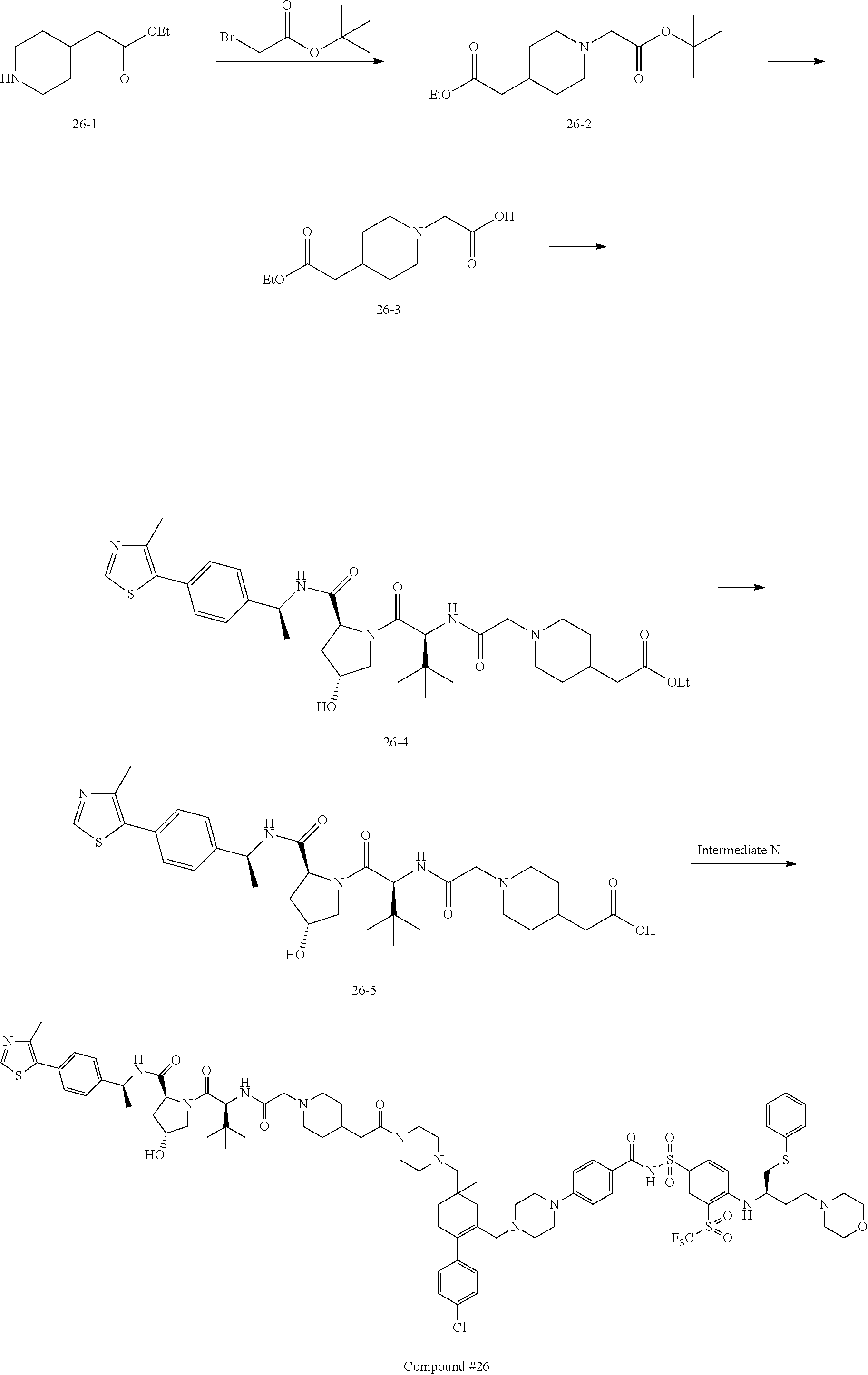

Compound #26 was prepared by following General Procedure C. tert-butyl 2-(4-(2-ethoxy-2-oxoethyl)piperidin-1-yl)acetate (26-2). ¹H NMR (600 MHz, Chloroform-d) δ 4.12 (q, J=7.1 Hz, 2H), 3.09 (s, 2H), 2.92 (d, J=11.6 Hz, 2H), 2.22 (d, J=7.1 6 Hz, 2H), 2.20-2.14 (m, 2H), 1.77 (ddd, J=11.4, 7.6, 4.0 Hz, 1H), 1.71-1.66 (m, 2H), 1.45 (s, 9H), 1.42-1.34 (m, 2H), 1.25 (t, J=7.1 Hz, 3H) ppm.

2-(4-(2-ethoxy-2-oxoethyl)piperidin-1-yl)acetic acid (26-3). LC/MS (ESI) m/z 230.1; [M+H]+ calcd for C₁₁H₂₀NO₄⁺: 230.14.

ethyl 2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)acetate (26−4). ¹H NMR (600 MHz, Chloroform-d) δ 8.67 (s, 1H), 7.89 (d, J=8.0 Hz, 1H), 7.50 (d, J=7.8 Hz, 1H), 7.40 (d, J=8.3 Hz, 2H), 7.36 (d, J=8.3 Hz, 2H), 5.07 (p, J=7.0 Hz, 1H), 4.76 (t, J=7.9 Hz, 1H), 4.50 (s, 1H), 4.40 (d, J=8.1 Hz, 1H), 4.21 (d, J=11.6 Hz, 1H), 4.13 (q, J=7.1 Hz, 2H), 3.57 (dd, J=11.4, 3.6 Hz, 1H), 3.04-2.94 (m, 2H), 2.84 (d, J=11.3 Hz, 2H), 2.59-2.54 (m, 1H), 2.53 (s, 3H), 2.28-2.16 (m, 4H), 2.06 (ddt, J=13.6, 8.3, 2.0 Hz, 1H), 1.84-1.72 (m, 4H), 1.47 (d, J=6.9 Hz, 3H), 1.37-1.27 (m, 2H), 1.26 (t, J=7.1 Hz, 3H), 1.07 (s, 9H) ppm.

2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)acetic acid (26-5). ¹H NMR (600 MHz, Methanol-d₄) δ 8.80 (s, 1H), 7.35 (q, J=8.4 Hz, 4H), 4.93 (q, J=5.6, 4.6 Hz, 1H), 4.55 (s, 1H), 4.52-4.43 (m, 2H), 4.36 (dt, J=4.1, 2.1 Hz, 1H), 3.79 (dt, J=11.1, 1.8 Hz, 1H), 3.67 (dd, J=11.0, 3.9 Hz, 1H), 3.55-3.35 (m, 1H), 3.16-3.09 (m, 2H), 2.58 (s, 1H), 2.40 (s, 3H), 2.20 (dd, J=15.3, 6.7 Hz, 2H), 2.13 (ddt, J=13.2, 7.6, 1.9 Hz, 1H), 1.91-1.79 (m, 4H), 1.51 (d, J=7.1 Hz, 1H), 1.44 (d, J=7.0 Hz, 5H), 0.98 (s, 9H) ppm. LC/MS (ESI) m/z 628.3; [M+H]⁺ calcd for C₃₂H₄₆N₅O₆S⁺: 628.32.

(2S,4R)-1-((2S)-2-(2-(4-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #26): ¹H NMR (600 MHz, Chloroform-d) δ 8.67 (d, J=2.7 Hz, 1H), 8.32 (t, J=2.5 Hz, 1H), 8.10 (dd, J=8.2, 4.4 Hz, 1H), 7.90 (t, J=9.0 Hz, 1H), 7.71 (t, J=7.8 Hz, 2H), 7.41-7.35 (m, 7H), 7.32-7.27 (m, 5H), 7.05 (d, J=8.5 Hz, 1H), 7.01-6.96 (m, 2H), 6.77 (d, J=8.5 Hz, 2H), 6.60 (d, J=9.4 Hz, 1H), 5.08 (p, J=7.0 Hz, 1H), 4.77 (t, J=7.9 Hz, 1H), 4.51 (d, J=8.7 Hz, 2H), 4.23 (d, J=11.5 Hz, 1H), 3.90 (s, 1H), 3.69-3.62 (m, 5H), 3.60 (d, J=10.8 Hz, 1H), 3.41 (s, 2H), 3.23 (s, 4H), 3.10 (dd, J=13.9, 5.1 Hz, 1H), 3.01 (dd, J=13.8, 7.5 Hz, 2H), 2.96 (t, J=5.2 Hz, 1H), 2.83 (d, J=11.9 22 Hz, 3H), 2.78 (d, J=12.5 Hz, 1H), 2.51 (d, J=0.8 Hz, 3H), 2.41 (d, J=10.9 Hz, 2H), 2.34-2.10 (m, 17H), 1.91-1.57 (m, 19H), 1.47 (dd, J=7.0, 4.0 Hz, 4H), 1.07 (s, 9H) ppm. LC/MS (ESI) m/z 1667.7; [M+H]⁺ calcd for C₈₄H₁₀₈ClF₃N₁₁O₁₁S₄⁺: 1667.67.

### Example 14: Preparation of Compound #27

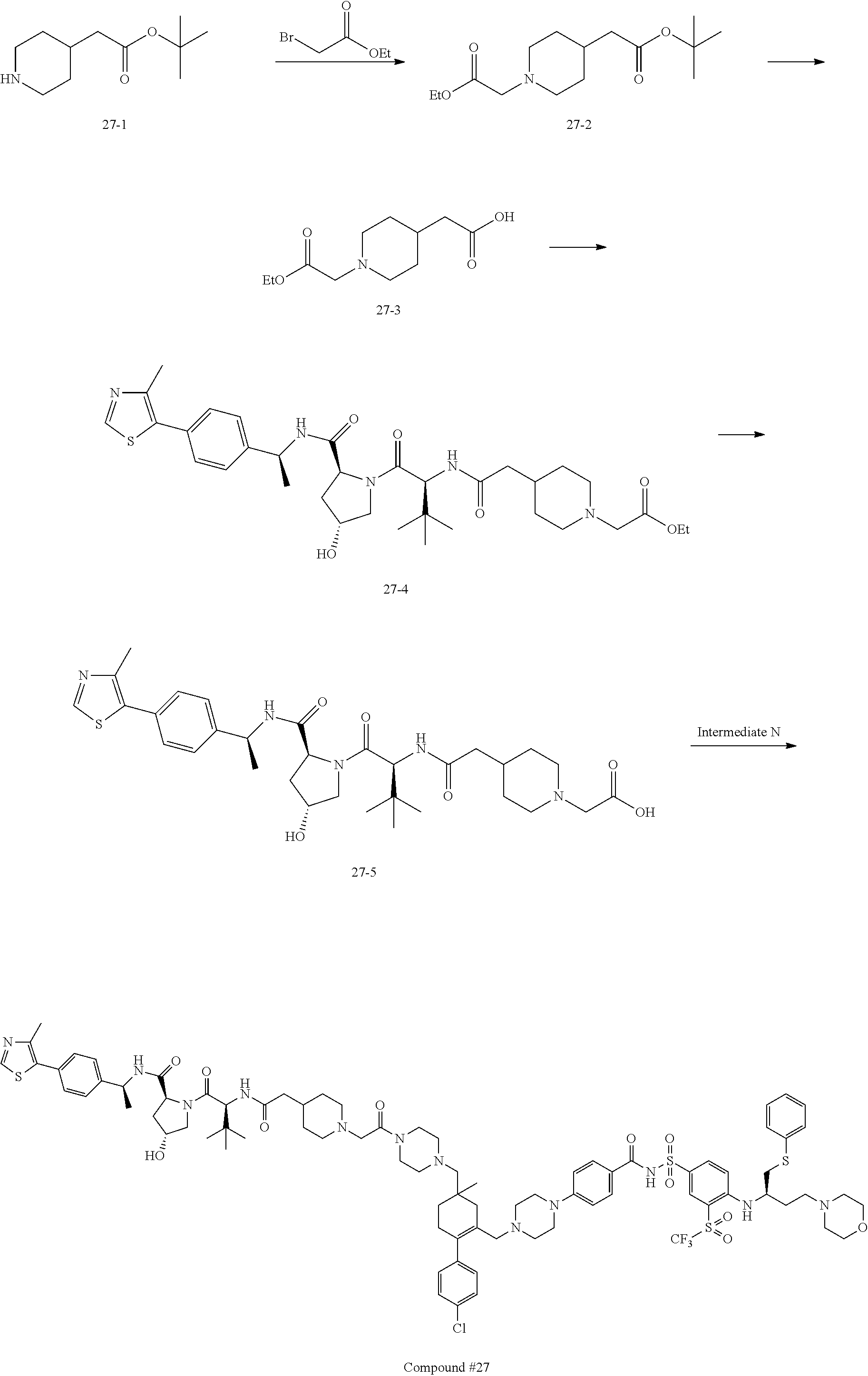

Compound #27 was prepared by following General Procedure C.

tert-butyl 2-(1-(2-ethoxy-2-oxoethyl)piperidin-4-yl)acetate (27-2). ¹H NMR (600 MHz, Chloroform-d) δ 4.18 (q, J=7.2 Hz, 2H), 3.18 (s, 2H), 2.91 (d, J=11.6 Hz, 2H), 2.20-2.15 (m, 2H), 2.13 (d, J=7.0 Hz, 2H), 1.74 (tt, J=7.2, 3.9 Hz, 1H), 1.71-1.67 (m, 2H), 1.44 (s, 9H), 1.41-1.34 (m, 2H), 1.26 (t, J=7.1 Hz, 3H) ppm.

2-(1-(2-ethoxy-2-oxoethyl)piperidin-4-yl)acetic acid (27-3). ¹H NMR (600 MHz, Chloroform-d) δ 4.27 (q, J=7.1 Hz, 2H), 3.93 (s, 2H), 3.81-3.74 (m, 2H), 3.13 (t, J=12.6 Hz, 2H), 2.42 (d, J=6.5 Hz, 2H), 2.04 (d, J=14.9 Hz, 3H), 1.87 (t, J=13.4 Hz, 2H), 1.29 (d, J=7.1 Hz, 3H) ppm.

ethyl 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)acetate (27-4). ¹H NMR (600 MHz, Chloroform-d) δ 8.67 (s, 1H), 7.45-7.39 (m, 3H), 7.36 (d, J=8.3 Hz, 2H), 6.09 (d, J=8.5 Hz, 1H), 5.08 (p, J=7.1 Hz, 1H), 4.74 (t, J=7.9 Hz, 1H), 4.52 (d, J=8.6 Hz, 2H), 4.17 (q, J=7.1 Hz, 2H), 4.14 (d, J=11.5 Hz, 1H), 3.58 (dd, J=11.4, 3.7 Hz, 1H), 3.18 (s, 2H), 2.92 (dd, J=10.7, 4.3 Hz, 2H), 2.58 (ddd, J=13.6, 7.5, 4.7 Hz, 1H), 2.53 (s, 3H), 2.20-2.13 (m, 3H), 2.13-2.04 (m, 2H), 1.78 (td, J=7.4, 3.7 Hz, 1H), 1.70-1.64 (m, 3H), 1.47 (d, J=7.0 Hz, 3H), 1.43-1.36 (m, 3H), 1.26 (t, J=7.1 Hz, 3H), 1.05 (s, 9H) ppm.

2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)acetic acid (27-5). ¹H NMR (600 MHz, Methanol-d₄) δ 8.79 (d, J=1.3 Hz, 1H), 8.48 (d, J=7.5 Hz, 1H), 7.86 (d, J=8.8 Hz, 1H), 7.38-7.31 (m, 4H), 4.92 (p, J=7.1 Hz, 1H), 4.53 (d, J=8.8 Hz, 1H), 4.47 (d, J=4.6 Hz, 1H), 4.35 (dt, J=4.4, 2.2 Hz, 1H), 3.80 (dt, J=11.1, 1.8 Hz, 1H), 3.66 (dd, J=11.0, 4.0 Hz, 1H), 3.59-3.47 (m, 4H), 2.96 (s, 2H), 2.39 (d, J=1.6 Hz, 3H), 2.23 (d, J=7.0 Hz, 2H), 2.16-2.09 (m, 1H), 1.98 (s, 1H), 1.87 (dq, J=13.3, 4.5 Hz, 3H), 1.57-1.48 (m, 2H), 1.43 (d, J=7.1 Hz, 3H), 0.97 (s, 9H) ppm.

(2S,4R)-1-((2S)-2-(2-(1-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)piperidin-4-yl)acetamido)-3,3-dimethylbutanoyl)-4-hydroxy-N—((S)-1-(4-(4-methylthiazol-5-yl)phenyl)ethyl)pyrrolidine-2-carboxamide (compound #27). ¹H NMR (600 MHz, Chloroform-d) δ 8.65 (d, J=1.7 Hz, 1H), 8.28 (t, J=2.1 Hz, 1H), 8.06-8.03 (m, 1H), 7.74 (dd, J=17.3, 8.5 Hz, 2H), 7.51 (d, J=7.7 Hz, 1H), 7.38-7.31 (m, 7H), 7.26-7.20 (m, 5H), 6.96 (dt, J=5.9, 1.8 Hz, 3H), 6.79 (s, 1H), 6.75 (d, J=8.6 Hz, 2H), 6.56 (d, J=9.6 Hz, 1H), 5.04 (p, J=7.0 Hz, 1H), 4.60 (t, J=8.2 Hz, 1H), 4.57-4.53 (m, 1H), 4.43 (s, 1H), 3.97 (d, J=11.5 Hz, 1H), 3.84 (s, 1H), 3.66-3.59 (m, 6H), 3.57 (d, J=11.0 Hz, 1H), 3.52 (s, 2H), 3.46 (s, 2H), 3.37 (p, J=1.7 Hz, 2H), 3.10-3.03 (m, 2H), 2.97 (d, J=23.4 Hz, 7H), 2.90-2.78 (m, 3H), 2.48 (d, J=1.7 Hz, 7H), 2.39 (s, 14H), 2.17-2.05 (m, 7H), 1.65 (dd, J=15.1, 9.4 Hz, 5H), 1.48-1.38 (m, 8H), 1.00 (d, J=1.7 Hz, 9H) ppm. LC/MS (ESI) m/z 1667.6; [M+H]⁺ calcd for C₈₃H₁₀₇ClF₃N₁₂O₁₁S₄⁺: 1667.67.

### Example 15: Preparation of Compounds #28−30

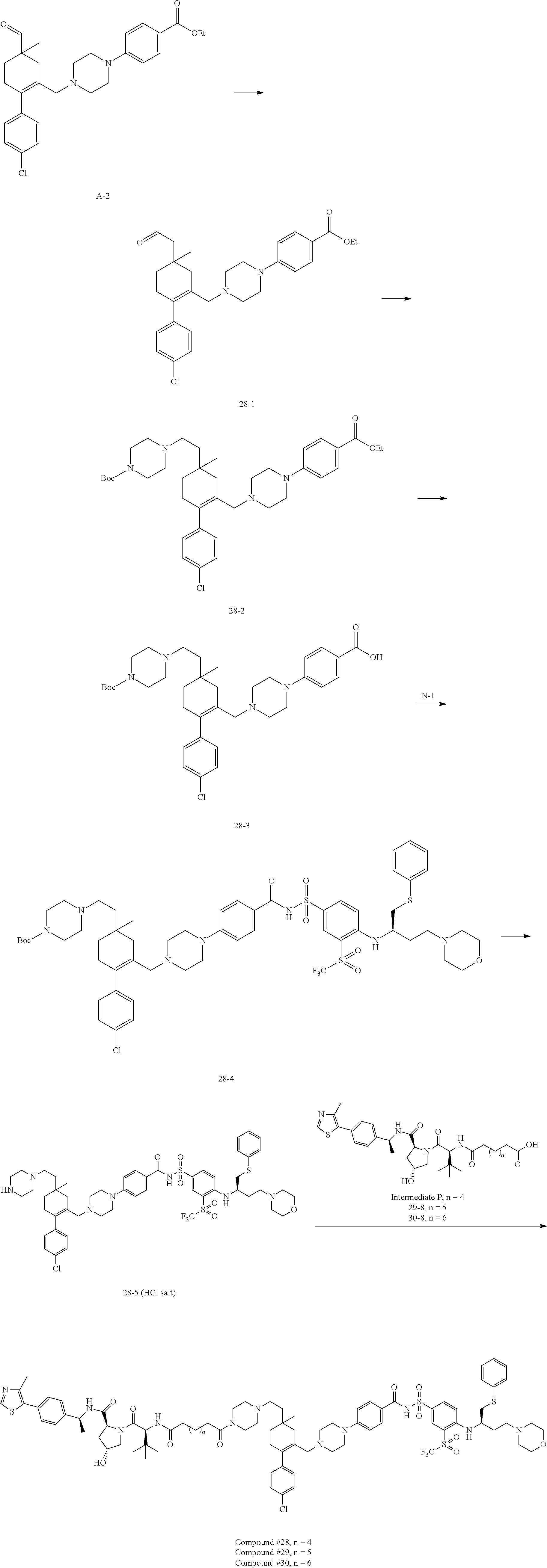

Preparation of ethyl 4-(4-((4′-chloro-4-methyl-4-(2-oxoethyl)-3,4,5,6-tetrahydro-[1,1′-biphenyl]-2-yl)methyl)piperazin-1-yl)benzoate (28-1). To a stirring solution of A-2 (1 equiv.) and (methoxymethyl)triphenylphosphonium chloride (1.5 equiv.) in DMF was added KOₜ-Bu (1.4 equiv), and the resulting reaction was stirred at room temperature for 5 hours. Once the starting material consumed the reaction was quenched with 1N HCl solution followed by addition of 3N HCl solution and the reaction was stirred overnight. Once the enol ether disappeared, the mixture was diluted with EtOAc and washed with water followed by brine solution. The organic part was dried over Na₂SO₄, filtered, and concentrated. The residue was purified by flash column chromatography to afford 28-1. ¹H NMR (600 MHz, Chloroform-d) δ 9.93 (s, 1H), 7.89 (d, J=9.0 Hz, 2H), 7.29 (d, J=8.4 Hz, 2H), 6.99 (d, J=8.4 Hz, 2H), 6.81 (d, J=9.0 Hz, 2H), 4.32 (q, J=7.1 Hz, 2H), 3.25 (t, J=5.1 Hz, 4H), 2.80 (s, 2H), 2.39 (d, J=3.1 Hz, 2H), 2.35 (t, J=5.1 14 Hz, 4H), 2.33-2.27 (m, 2H), 2.26 (s, 1H), 2.16-2.10 (m, 1H), 1.74-1.68 (m, 1H), 1.61 (dt, J=13.1, 6.5 Hz, 3H), 1.36 (t, J=7.1 Hz, 3H), 1.19 (s, 3H). ¹³C NMR (151 MHz, Chloroform-d) δ 203.42, 166.84, 154.27, 141.32, 134.96, 132.49, 131.26, 129.79, 129.01, 128.55, 120.06, 113.68, 60.53, 60.46, 53.47, 52.66, 47.70, 40.17, 34.35, 32.31, 30.19, 25.98, 14.61 ppm.

Compounds #28-30 were prepared by following General Procedure F. tert-butyl 4-(2-(4′-chloro-6-((4-(4-(ethoxycarbonyl)phenyl)piperazin-1-yl)methyl)-4-methyl-2,3,4,5-tetrahydro-[1,1′-biphenyl]-4-yl)ethyl)piperazine-1-carboxylate (28-2). ¹H NMR (600 22 MHz, Chloroform-d) δ 7.89 (d, J=9.0 Hz, 2H), 7.27 (d, J=8.0 Hz, 2H), 6.98 (d, J=8.4 Hz, 2H), 6.80 (d, J=9.1 Hz, 2H), 4.31 (q, J=7.1 Hz, 2H), 3.58 (t, J=5.4 Hz, 1H), 3.47-3.38 (m, 7H), 3.24 (t, J=5.1 Hz, 4H), 2.79 (s, 2H), 2.42 (d, J=7.6 Hz, 6H), 2.34 (t, J=5.2 Hz, 4H), 2.24 (d, J=5.8 Hz, 2H), 2.10 (s, 2H), 1.45 (s, 9H), 1.35 (t, J=7.1 Hz, 3H), 0.98 (s, 3H) ppm.

4-(4-((4-(2-(4-(tert-butoxycarbonyl)piperazin-1-yl)ethyl)-4′-chloro-4-methyl-3,4,5,6-tetrahydro-[1,1′-biphenyl]-2-yl)methyl)piperazin-1-yl)benzoic acid (28-3). ¹H NMR (600 6 MHz, Chloroform-d) δ 7.88 (d, J=9.0 Hz, 2H), 7.20 (d, J=8.4 Hz, 2H), 6.96 (d, J=8.5 Hz, 2H), 6.73 (d, J=9.2 Hz, 2H), 3.60-3.55 (m, 4H), 3.45-3.38 (m, 2H), 3.21 (d, J=5.4 Hz, 4H), 2.83 (s, 2H), 2.69 (s, 4H), 2.65-2.57 (m, 2H), 2.43-2.37 (m, 2H), 2.34-2.27 (m, 3H), 2.22-2.15 (m, 2H), 2.00-1.96 (m, 1H), 1.77 (dt, J=12.6, 6.2 Hz, 1H), 1.44 (s, 9H), 0.97 (s, 3H) ppm.

tert-butyl 4-(2-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)ethyl)piperazine-1-carboxylate (28-4). ¹H NMR (600 MHz, Chloroform-d) δ 8.28 (d, J=2.3 Hz, 1H), 7.98-7.92 (m, 1H), 7.86 (d, J=8.4 Hz, 2H), 7.34 (d, J=7.1 Hz, 2H), 7.25-7.19 (m, 4H), 7.00 (d, J=8.0 Hz, 2H), 6.80 (d, J=8.5 16 Hz, 1H), 6.67 (d, J=8.5 Hz, 2H), 6.48 (d, J=9.3 Hz, 1H), 3.81 (dt, J=8.7, 4.5 Hz, 1H), 3.62 (dt, J=11.0, 4.9 Hz, 8H), 3.20 (t, J=5.1 Hz, 4H), 3.07 (dd, J=13.8, 4.7 Hz, 1H), 2.98-2.90 (m, 3H), 2.78 (s, 5H), 2.54-2.38 (m, 6H), 2.38-2.26 (m, 6H), 2.26-2.16 (m, 2H), 2.13-2.06 (m, 1H), 2.01 (d, J=17.6 Hz, 1H), 1.73-1.66 (m, 2H), 1.64 (dd, J=6.0, 2.8 Hz, 2H), 1.48 (dq, J=13.4, 6.7 Hz, 2H), 1.43 (s, 9H), 0.95 (s, 3H) ppm. LC/MS (ESI) m/z 1172.4; [M+H]⁺ calcd for C₅₇H₇₃ClF₃N₇O₈S₃⁺: 1172.44.

4-(4-((4′-chloro-4-methyl-4-(2-(piperazin-1-yl)ethyl)-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (28-5). LC/MS (ESI) m/z 1072.4; [M+H]⁺ calcd for C₅₂H₆₆ClF₃N₇O₆S₃⁺: 1072.39.

(2S,4R)-1-((2S)-2-(8-(4-(2-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)ethyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #28). ¹H NMR (600 MHz, Chloroform-d) δ 8.67 (s, 1H), 8.32 (dd, J=11.0, 2.3 Hz, 1H), 8.08 (d, J=9.3 Hz, 1H), 7.80 (d, J=8.2 Hz, 1H), 7.73 (d, J=8.5 Hz, 1H), 7.42-7.28 (m, 11H), 7.17 (s, 2H), 6.99 (d, J=8.3 Hz, 1H), 6.73 (s, 1H), 6.65-6.36 (m, 1H), 5.08 (dt, J=14.1, 7.3 Hz, 1H), 4.72-4.55 (m, 2H), 4.51 (s, 1H), 4.06-3.89 (m, 4H), 3.66 (d, J=26.8 Hz, 6H), 3.60 (d, J=11.2 Hz, 2H), 3.44 (d, J=50.2 Hz, 4H), 3.35 (s, 2H), 3.12-3.09 (m, 1H), 3.04-3.01 (m, 1H), 2.96 (t, J=7.8 Hz, 2H), 2.57 (dd, J=27.0, 7.5 Hz, 2H), 2.50 (d, J=2.3 Hz, 4H), 2.43-2.19 (m, 13H), 2.17 (s, 5H), 1.76 (t, J=7.6 Hz, 6H), 1.56 (d, J=7.3 Hz, 10H), 1.45 (dd, J=28.7, 6.9 Hz, 6H), 1.04-1.01 (m, 12H) ppm. LC/MS (ESI) m/z 1654.5; [M+H]⁺ calcd for C₈₃H₁₀₈ClF₃N₁₁O₁₁S₄⁺: 1654.67.

(2S,4R)-1-((2S)-2-(9-(4-(2-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)ethyl)piperazin-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (compound #29). ¹H NMR (600 MHz, Chloroform-d) δ 8.67 (s, 1H), 8.31 (t, J=2.5 Hz, 1H), 8.06 (dt, J=7.2, 2.8 Hz, 1H), 7.75 (dd, J=8.7, 5.7 Hz, 2H), 7.41−7.28 (m, 9H), 6.98 (d, J=8.2 Hz, 3H), 6.74 (d, J=8.6 Hz, 2H), 6.57 (dd, J=9.5, 3.1 Hz, 1H), 6.34 (dd, J=54.7, 8.8 Hz, 1H), 5.07 (dt, J=14.2, 7.2 Hz, 1H), 4.71 (dt, J=12.9, 8.0 Hz, 1H), 4.60 (dd, J=14.9, 8.8 Hz, 1H), 4.50 (s, 1H), 4.14-4.05 (m, 1H), 3.90-3.82 (m, 1H), 3.70-3.56 (m, 8H), 3.52 (s, 2H), 3.21 (s, 4H), 3.10 (dd, J=13.8, 5.0 Hz, 1H), 2.99 (dd, J=13.9, 7.4 Hz, 1H), 2.92 (s, 1H), 2.80 (s, 2H), 2.51 (d, J=2.9 Hz, 8H), 2.46-2.33 (m, 8H), 2.28 (ddd, J=12.0, 8.2, 4.4 Hz, 7H), 2.26-2.15 (m, 5H), 2.11 (dt, J=13.2, 7.0 Hz, 2H), 1.69-1.50 (m, 10H), 1.45 (dd, J=18.8, 6.9 Hz, 6H), 1.29-1.27 (m, 6H), 1.04 (d, J=3.1 Hz, 9H), 0.98-0.96 (m, 3H) ppm. LC/MS (ESI) m/z 1668.6; [M+H]⁺ calcd for C₈₄H₁₁₀ClF₃N₁₁O₁₁S₄⁺: 1668.69. (2S,4R)-1-((2S)-2-(10-(4-(2-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)ethyl)piperazin-1-yl)-10-oxodecanamido)-3,3-dimethylbutanoyl)-4-hydroxy-N—((S)-1-(4-(4-methylthiazol-5-yl)phenyl)ethyl)pyrrolidine-2-carboxamide (compound #30). ¹H NMR (600 MHz, Chloroform-d) δ 8.67 (s, 1H), 8.31 (d, J=2.2 Hz, 1H), 8.06 (d, J=9.1 Hz, 1H), 7.73 (dd, J=8.8, 1.6 Hz, 2H), 7.39−7.34 (m, 6H), 7.30 (d, J=7.1 Hz, 2H), 7.25-7.24 (m, 1H), 6.98 (t, J=7.6 Hz, 3H), 6.74 (d, J=8.7 Hz, 2H), 6.60-6.56 (m, 1H), 6.23 (dd, J=16.2, 8.7 Hz, 1H), 5.08 (td, J=7.3, 4.3 Hz, 1H), 4.72 (q, J=7.6 Hz, 1H), 4.57 (dd, J=8.8, 5.4 Hz, 1H), 4.54-4.45 (m, 1H), 4.15-4.05 (m, 1H), 3.89 (dd, J=8.6, 4.8 Hz, 1H), 3.73-3.63 (m, 6H), 3.63-3.51 (m, 4H), 3.21 (t, J=5.2 Hz, 4H), 3.10 (dd, J=13.8, 5.0 Hz, 1H), 3.00 (dd, J=13.8, 7.3 Hz, 1H), 2.81 (s, 2H), 2.55 (d, J=8.0 Hz, 4H), 2.51 (s, 4H), 2.42 (s, 2H), 2.40-2.33 (m, 4H), 2.33-2.27 (m, 7H), 2.23-2.18 (m, 2H), 1.97 (d, J=17.5 Hz, 1H), 1.69-1.63 (m, 1H), 1.58 (q, J=7.3 Hz, 6H), 1.52 (t, J=5.4 Hz, 2H), 1.46 (dd, J=6.9, 5.7 Hz, 3H), 1.26-1.24 (m, 16H), 1.04 (d, J=1.8 Hz, 9H), 0.97 (s, 3H) ppm. LC/MS (ESI) m/z 1682.6; [M+H]⁺ calcd for C₈₅H₁₁₂ClF₃N₁₁O₁₁S₄⁺: 1682.71.

### Example 16: Preparation of Compounds #31-32

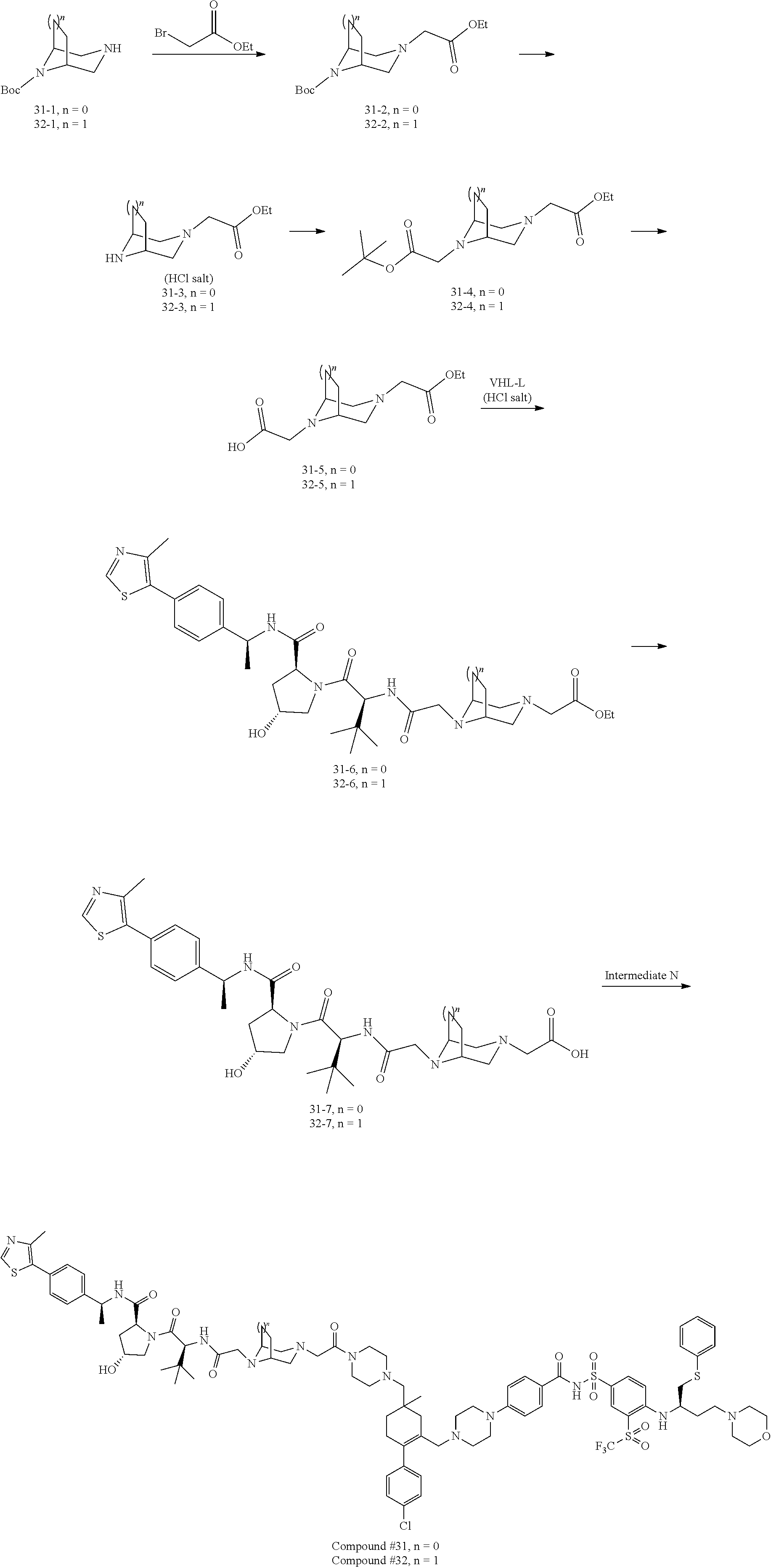

Compounds #31-32 were prepared by following General Procedure C.

tert-butyl (1R,5S)-3-(2-ethoxy-2-oxoethyl)-3,6-diazabicyclo[3.1.1]heptane-6-carboxylate (31-2). ¹H NMR (600 MHz, Chloroform-d) δ 4.17 (q, J=7.1 Hz, 2H), 4.05 (d, J=26.2 Hz, 2H), 3.39 (d, J=3.8 Hz, 2H), 3.36 (s, 1H), 3.21 (s, 1H), 3.01 (d, J=9.8 Hz, 2H), 2.43 (dtt, J=8.1, 6.1, 1.0 Hz, 1H), 1.61 (d, J=8.1 Hz, 1H), 1.44 (s, 9H), 1.27 (t, J=7.1 Hz, 3H) ppm.

tert-butyl (1R,5S)-3-(2-ethoxy-2-oxoethyl)-3,8-diazabicyclo[3.2.1]octane-8-carboxylate (32-2). ¹H NMR (600 MHz, Chloroform-d) δ 4.20 (s, 1H), 4.15 (q, J=7.1 Hz, 2H), 4.13-4.07 (m, 1H), 3.19 (d, J=1.5 Hz, 2H), 2.65 (d, J=10.6 Hz, 2H), 2.56 (d, J=34.3 Hz, 2H), 1.91 (dd, J=7.5, 4.7 Hz, 2H), 1.88-1.78 (m, 2H), 1.46 (s, 9H), 1.25 (s, 3H) ppm.

tert-butyl 2-((1R,5S)-3-(2-ethoxy-2-oxoethyl)-3,6-diazabicyclo[3.1.1]heptan-6-yl)acetate (31-3). ¹H NMR (600 MHz, Chloroform-d) δ 4.19 (q, J=7.1 Hz, 2H), 3.73 (d, J=5.8 Hz, 2H), 3.45 (s, 2H), 3.40 (s, 2H), 3.26 (d, J=10.6 Hz, 2H), 2.89 (dd, J=10.8, 1.5 Hz, 2H), 2.56 (dt, J=8.0, 5.9 Hz, 1H), 1.89 (d, J=8.1 Hz, 1H), 1.45 (s, 10H), 1.29 (t, J=7.1 Hz, 3H) ppm.

tert-butyl 2-((1R,5S)-3-(2-ethoxy-2-oxoethyl)-3,8-diazabicyclo[3.2.1]octan-8-yl)acetate (32-3). ¹H NMR (600 MHz, Chloroform-d) δ 4.14 (q, J=7.1 Hz, 2H), 3.24 (s, 2H), 3.16 (s, 2H), 3.14 (s, 2H), 2.69-2.58 (m, 4H), 1.94-1.82 (m, 4H), 1.45 (s, 9H), 1.26 (t, J=7.1 Hz, 3H) ppm.

tert-butyl 2-((1R,5S)-3-(2-ethoxy-2-oxoethyl)-3,6-diazabicyclo[3.1.1]heptan-6-yl)acetate (31-4). LC/MS (ESI) m/z 299.1; [M+H]⁺ calcd for C₁₅H₂₇N₂O₄⁺: 299.20.

2-((1R,5S)-3-(2-ethoxy-2-oxoethyl)-3,8-diazabicyclo[3.2.1]octan-8-yl)acetic acid (32-4). ¹H NMR (600 MHz, Methanol-d₄) δ 4.24-4.18 (m, 4H), 4.10 (s, 2H), 3.72 (s, 2H), 3.64 (s, 1H), 3.42 (d, J=13.2 Hz, 2H), 3.35-3.30 (m, 2H), 2.32-2.27 (m, 4H), 1.27 (t, J=7.1 Hz, 3H) ppm.

ethyl 2-((1R,5S)-6-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-3,6-diazabicyclo[3.1.1]heptan-3-yl)acetate (31-5). ¹H NMR (600 MHz, Chloroform-d) δ 8.67-8.64 (m, 1H), 8.10 (s, 1H), 7.51 (d, J=7.8 Hz, 1H), 7.36 (ddd, J=8.8, 6.3, 3.7 Hz, 4H), 5.09 (d, J=11.8 Hz, 1H), 4.73 (d, J=6.3 Hz, 1H), 4.55-4.44 (m, 2H), 4.20-4.02 (m, 3H), 3.58 (dd, J=11.4, 3.6 Hz, 1H), 3.45 (s, 2H), 3.32 (d, J=20.4 Hz, 1H), 3.11 (d, J=2.0 Hz, 6H), 3.03-2.96 (m, 2H), 2.53-2.48 (m, 4H), 2.08 (dd, J=13.5, 8.1 Hz, 1H), 2.05-1.96 (m, 1H), 1.47 (dd, J=7.2, 3.0 Hz, 4H), 1.21 (s, 3H), 1.06 (s, 9H) ppm.

ethyl 2-((1R,5S)-8-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-3,8-diazabicyclo[3.2.1]octan-3-yl)acetate (32-5). ¹H NMR (600 MHz, Chloroform-d) δ 8.67 (s, 1H), 8.28 (s, 1H), 7.53 (d, J=7.7 Hz, 1H), 7.40 (d, J=8.0 Hz, 2H), 7.36 (d, J=8.2 Hz, 2H), 5.07 (p, J=7.0 Hz, 1H), 4.78 (dt, J=8.0, 3.6 Hz, 1H), 4.49 (s, 1H), 4.40 (d, J=7.6 Hz, 1H), 4.23 (d, J=12.1 Hz, 1H), 4.17-4.13 (m, 2H), 3.57 (d, J=11.4 Hz, 1H), 3.20 (s, 3H), 3.09 (s, 2H), 2.99 (d, J=11.5 Hz, 2H), 2.73-2.63 (m, 3H), 2.53 (s, 3H), 2.10-2.05 (m, 1H), 1.91 (d, J=7.9 Hz, 2H), 1.82 (q, J=7.7, 6.3 Hz, 2H), 1.47 (d, J=6.9 Hz, 3H), 1.27 (td, J=7.0, 1.5 Hz, 3H), 1.09 (s, 9H) ppm.

(2S,4R)-1-((2S)-2-(2-(3-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)-3,6-diazabicyclo[3.1.1]heptan-6-yl)acetamido)-3,3-dimethylbutanoyl)-4-hydroxy-N—((S)-1-(4-(4-methylthiazol-5-yl)phenyl)ethyl)pyrrolidine-2-carboxamide (compound #31). ¹H NMR (600 4 MHz, Chloroform-d) δ 8.66 (d, J=1.1 Hz, 1H), 8.32 (dd, J=6.5, 2.2 Hz, 2H), 8.05 (t, J=8.7 Hz, 2H), 7.89 (d, J=8.4 Hz, 1H), 7.84 (s, 1H), 7.37 (dt, J=8.1, 1.3 Hz, 6H), 7.29-7.27 (m, 5H), 7.01-6.98 (m, 3H), 6.89 (d, J=8.5 Hz, 1H), 6.81-6.73 (m, 4H), 6.55 (d, J=9.2 Hz, 1H), 6.50 (d, J=9.3 Hz, 1H), 5.08 (q, J=7.1 Hz, 1H), 4.80 (t, J=8.2 Hz, 1H), 4.52 (d, J=8.3 Hz, 1H), 4.46 (s, 1H), 4.13 (d, J=10.3 Hz, 1H), 3.85 (s, 1H), 3.64 (s, 8H), 3.58 (d, J=11.1 Hz, 1H), 3.34 (s, 4H), 3.22 (d, J=35.3 Hz, 5H), 3.12-3.07 (m, 1H), 2.96 (d, J=7.3 Hz, 15H), 2.67 (s, 2H), 2.51 (d, J=0.7 Hz, 4H), 2.43 (s, 2H), 2.35 (td, J=16.4, 15.2, 8.6 Hz, 6H), 2.23 (d, J=19.6 Hz, 1H), 2.16-2.07 (m, 1H), 1.73-1.52 (m, 4H), 1.48 (d, J=6.9 Hz, 3H), 1.35 (d, J=18.6 Hz, 5H), 1.06 (s, 9H) ppm. LC/MS (ESI) m/z 1680.7; [M+H]⁺ calcd for C₈₃H₁₀₆ClF3N₁₃O₁₁S₄⁺: 1680.67.

(2S,4R)-1-((2S)-2-(2-((1R,5S)-3-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)-3,8-diazabicyclo[3.2.1]octan-8-yl)acetamido)-3,3-dimethylbutanoyl)-4-hydroxy-N—((S)-1-(4-(4-methylthiazol-5-yl)phenyl)ethyl)pyrrolidine-2-carboxamide (compound #32). ¹H NMR (600 MHz, Chloroform-d) δ 8.67 (s, 1H), 8.32 (t, J=2.1 Hz, 1H), 8.15 (s, 1H), 8.09 (t, J=9.8 Hz, 1H), 7.69 (d, J=8.4 Hz, 2H), 7.47 (d, J=8.9 Hz, 1H), 7.42−7.34 (m, 6H), 7.32-7.26 (m, 4H), 7.02 (d, J=8.6 Hz, 1H), 7.00-6.97 (m, 2H), 6.77 (d, J=8.5 Hz, 2H), 6.63-6.58 (m, 1H), 5.08 (p, J=7.2 Hz, 1H), 4.76 (td, J=7.9, 5.9 Hz, 1H), 4.53-4.44 (m, 2H), 4.22 (s, 1H), 3.90 (s, 1H), 3.65 (td, J=6.4, 3.4 Hz, 5H), 3.57 (s, 4H), 3.21 (d, J=26.3 Hz, 5H), 3.17-2.98 (m, 6H), 2.81 (d, J=29.1 Hz, 4H), 2.66-2.54 (m, 5H), 2.52 (s, 3H), 2.45-2.22 (m, 19H), 2.15-2.03 (m, 4H), 1.90 (d, J=16.6 Hz, 2H), 1.73-1.56 (m, 7H), 1.48 (d, J=6.9 Hz, 3H), 1.07 (s, 10H) ppm. LC/MS (ESI) m/z 1694.6; [M+H]⁺ calcd for C₈₄H₁₀₈ClF₃N₁₃O₁₁S₄⁺: 1694.68.

### Example 17: Preparation of Compound #33

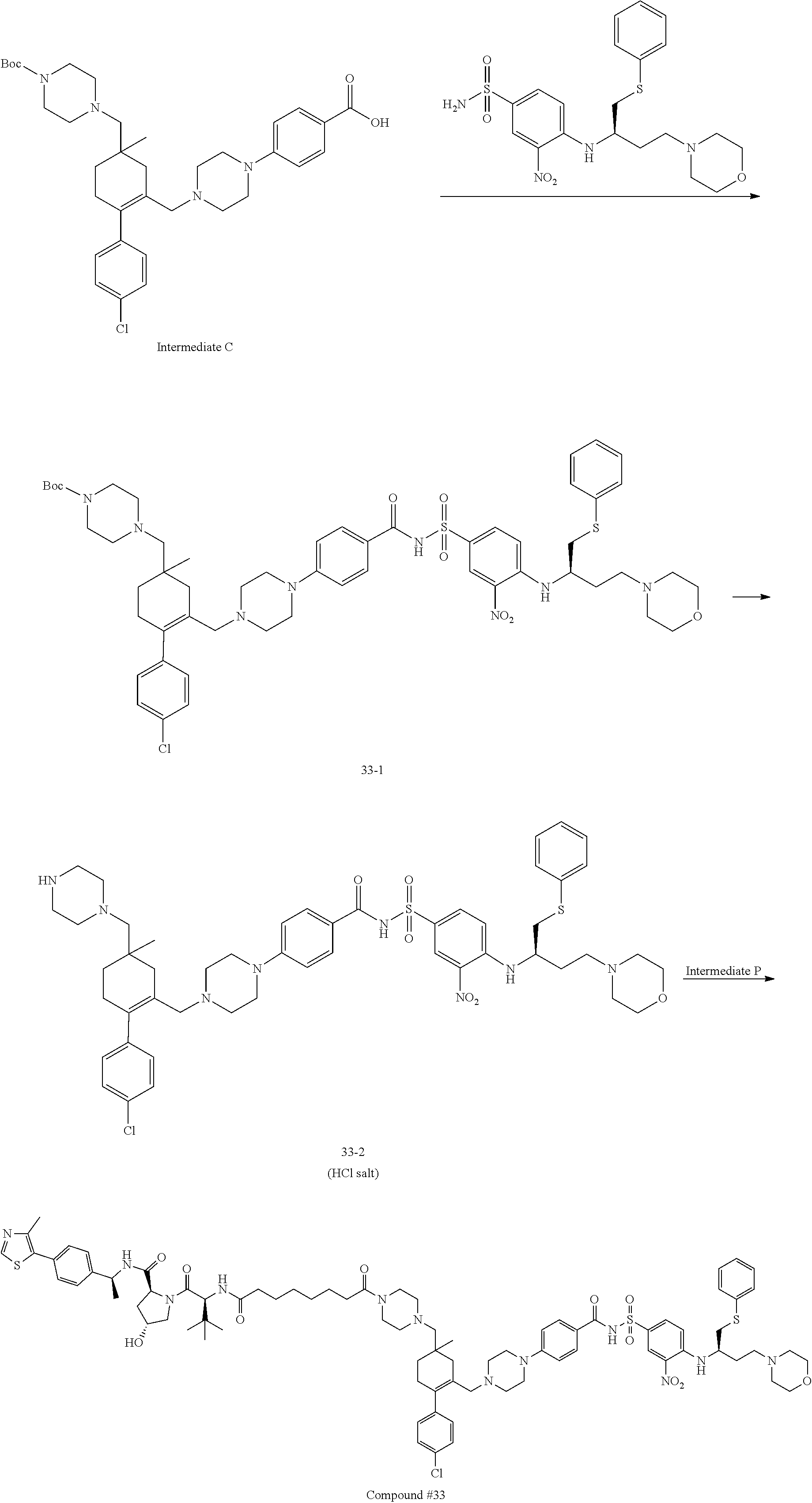

Compound #33 was prepared by following General Procedure E. tert-butyl 4-((4′-chloro-4-methyl-6-((4-(4-(((4-(((R)-4-morpholino-1-(phenylthio)butan-2-yl)amino)-3-nitrophenyl)sulfonyl)carbamoyl)phenyl)piperazin-1-yl)methyl)-2,3,4,5-tetrahydro-[1,1′-biphenyl]-4-yl)methyl)piperazine-1-carboxylate (33-1) (73 mg, 61% yield). ¹H NMR (600 MHz, CDCl₃) δ 8.75 (d, J=2.3 Hz, 1H), 7.99-7.92 (m, 1H), 7.73 (d, J=8.6 Hz, 2H), 7.34-7.17 (m, 8H), 7.02-6.94 (m, 2H), 6.74 (dd, J=20.2, 9.0 Hz, 3H), 3.99 (p, J=5.7 Hz, 1H), 3.69 (qt, J=10.0, 5.0 Hz, 4H), 3.38 (d, J=5.2 Hz, 4H), 3.26 (t, J=5.2 Hz, 4H), 3.19-3.08 (m, 2H), 2.87 (s, 2H), 2.56-2.34 (m, 16H), 2.33-2.25 (m, 1H), 2.24-2.17 (m, 3H), 2.17-2.07 (m, 2H), 1.93 (d, J=17.3 Hz, 1H), 1.87-1.77 (m, 1H), 1.62-1.55 (m, 1H), 1.44 (s, 9H), 0.93 (s, 3H) ppm. LC/MS (ESI) m/z 1071.7; [M+H]⁺ calcd for C₅₅H₇₂ClN₈O₈S₂⁺: 1071.46.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-morpholino-1-(phenylthio)butan-2-yl)amino)-3-nitrophenyl)sulfonyl)benzamide hydrochloride (33-2) (75 mg, quantitative yield). LC/MS (ESI) m/z 971.4; [M+H]⁺ calcd for C₅₀H₆₄ClN₈O₆S₂⁺: 971.41.

(2S,4R)-1-((2S)-2-(8-(4-((4′-chloro-4-methyl-6-((4-(4-(((4-(((R)-4-morpholino-1-(phenylthio)butan-2-yl)amino)-3-nitrophenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #33) (29.9 mg, 49% yield). ¹H NMR (600 MHz, Chloroform-d) δ 8.76 (d, J=2.5 Hz, 1H), 8.68 (s, 1H), 7.97 (d, J=9.2, 2.5 Hz, 1H), 7.74 (d, J=8.5 Hz, 2H), 7.54-7.45 (m, 1H), 7.42-7.31 (m, 6H), 7.31-7.18 (m, 5H), 7.02-6.96 (m, 2H), 6.74 (d, J=8.1 Hz, 3H), 6.52 (d, J=9.8 Hz, 1H), 5.15-5.04 (m, 1H), 4.74-4.64 (m, 1H), 4.60 (dd, J=9.1, 4.1 Hz, 1H), 4.49 (s, 1H), 4.07 (d, J=11.3 Hz, 1H), 4.01 (s, 1H), 3.76 2-3.63 (m, 5H), 3.64-3.51 (m, 3H), 3.43 (s, 2H), 3.31-3.19 (m, 5H), 3.19-3.09 (m, 3H), 2.87 (s, 2H), 2.66-2.07 (m, 25H), 1.97-1.76 (m, 3H), 1.68-1.53 (m, 6H), 1.53-1.41 (m, 4H), 1.38-1.24 (m, 6H), 1.04 (s, 9H), 0.96 (s, 3H) ppm. LC/MS (ESI) m/z 1553.7; [M+H]⁺ calcd for C₈₁H₁₀₆ClN₁₂O₁₁S₃⁺: 1553.69.

### Example 18: Preparation of Compound #34

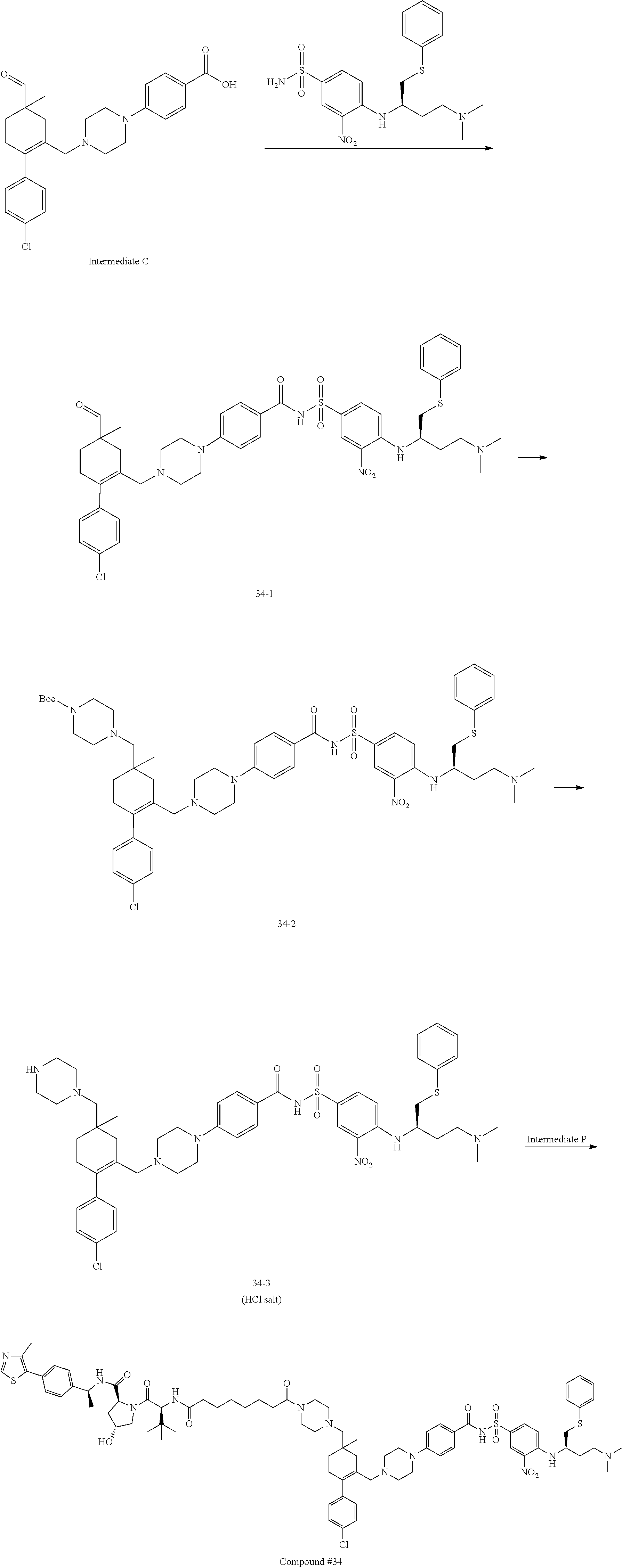

Preparation of 4-(4-((4′-chloro-4-formyl-4-methyl-3,4,5,6-tetrahydro-[1,1′-biphenyl]-2-yl)methyl)piperazin-1-yl)-N-((4-(((R)-4-(dimethylamino)-1-(phenylthio)butan-2-yl)amino)-3-nitrophenyl)sulfonyl)benzamide (34-1): A mixture of Intermediate G (100 mg, 0.22 mmol), (R)-4-((4-(dimethylamino)-1-(phenylthio)butan-2-yl)amino)-3-nitrobenzenesulfonamide (112 14 mg, 0.27 mmol), EDC (127 mg, 0.66 mmol), and DMAP (81 mg, 0.66 mmol) in DCM (5 mL) was stirred at room temperature overnight. The reaction mixture was washed with water and sat. aq. NH₄Cl, dried over Na₂SO₄, filtered, and concentrated. The residue was purified by flash column chromatography (0% to 20% of MeOH in DCM) to afford 34−1 (124 mg, 65% yield). LC/MS (ESI) m/z 859.5; [M+H]⁺ calcd for C₄₄H₅₂ClN₆O₆S₂⁺: 859.31.

The last three steps for the preparation of compound #34 were carried out according to General Procedure G.

tert-butyl 4-((4′-chloro-6-((4-(4-(((4-(((R)-4-(dimethylamino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (34-2) (23 mg, 48% yield). ¹H NMR (600 MHz, Chloroform-d) δ 8.64 (s, 1H), 7.84 (d, J=8.5 Hz, 3H), 7.33-7.24 (m, 4H), 7.22-7.11 (m, 3H), 6.99 (dd, J=8.5, 2.5 Hz, 2H), 6.77-6.70 (m, 2H), 6.67 (s, 1H), 4.05-3.94 (m, 1H), 3.48 (t, J=5.3 Hz, 1H), 3.46-3.35 (m, 4H), 3.28-3.17 (m, 4H), 3.11 (s, 2H), 2.83 (s, 4H), 2.65-2.45 (m, 9H), 2.45-2.26 (m, 5H), 2.25-2.09 (m, 5H), 2.07-1.89 (m, 3H), 1.64-1.54 (m, 1H), 1.46 (s, 9H), 0.95 (d, J=2.8 Hz, 3H) ppm. LC/MS (ESI) m/z 1029.8; [M+H]⁺ calcd for C₅₃H₇₀ClN₈O₇S₂⁺: 1029.45.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(dimethylamino)-1-(phenylthio)butan-2-yl)amino)-3-nitrophenyl)sulfonyl)benzamide hydrochloride (34-3) (25 mg, quantitative yield). ¹H NMR (600 MHz, Methanol-d₄) δ 8.70 (d, J=2.3 Hz, 1H), 7.98 (dd, J=9.2, 2.4 Hz, 1H), 7.80 (d, J=8.9 Hz, 2H), 7.43 (d, J=8.0 Hz, 2H), 7.27-7.20 (m, 4H), 7.10-7.05 (m, 4H), 7.02 (d, J=8.8 Hz, 2H), 4.23 (dq, J=9.8, 5.0 Hz, 1H), 3.97-3.82 (m, 3H), 3.82-3.41 (m, 15H), 3.37-3.21 (m, 4H), 3.03-2.95 (m, 1H), 2.90 (d, J=4.4 Hz, 6H), 2.87-2.70 (m, 2H), 2.51 (s, 2H), 2.41 (d, J=16.5 Hz, 1H), 2.36-2.28 (m, 1H), 2.27-2.19 (m, 1H), 2.14-2.04 (m, 1H), 1.68 (d, J=10.6 Hz, 1H), 1.27 (s, 3H) ppm. LC/MS (ESI) m/z 929.7; [M+H]⁺ calcd for C₄₈H₆₂ClN₈O₅S₂⁺: 929.40.

(2S,4R)-1-((2S)-2-(8-(4-((4′-chloro-6-((4-(4-(((4-(((R)-4-(dimethylamino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #34) (4.9 mg, 17% yield). ¹H NMR (600 MHz, Chloroform-d) δ 8.73-8.64 (m, 2H), 7.89 (d, 1H), 7.80 (d, J=8.4 Hz, 2H), 7.56-7.47 (m, 1H), 7.42-7.35 (m, 4H), 7.34-7.25 (m, 4H), 7.24-7.15 (m, 3H), 7.02-6.97 (m, 2H), 6.75 (d, J=8.6 Hz, 2H), 6.68 (d, J=9.2 Hz, 1H), 6.53 (d, J=8.9 Hz, 1H), 5.09 (p, J=7.1 Hz, 1H), 4.75-4.66 (m, 1H), 4.61 (dd, J=9.1, 3.5 Hz, 1H), 4.49 (s, 1H), 4.07 (d, J=11.4 Hz, 1H), 3.99 (s, 1H), 3.67-3.50 (m, 3H), 3.47-3.37 (m, 2H), 3.21 (s, 4H), 3.12 (d, J=5.6 Hz, 2H), 2.89-2.77 (m, 2H), 2.73-2.06 (m, 32H), 2.02-1.86 (m, 2H), 1.74-1.50 (m, 6H), 1.53-1.41 (m, 4H), 1.38-1.16 (m, 5H), 1.04 (s, 9H), 0.95 (s, 3H) ppm. LC/MS (ESI) m/z 1511.4; [M+H]+ calcd for C₇₉H₁₀₄ClN₁₂O₁₀S₃⁺: 1511.68.

### Example 19: Preparation of Compounds #35-36

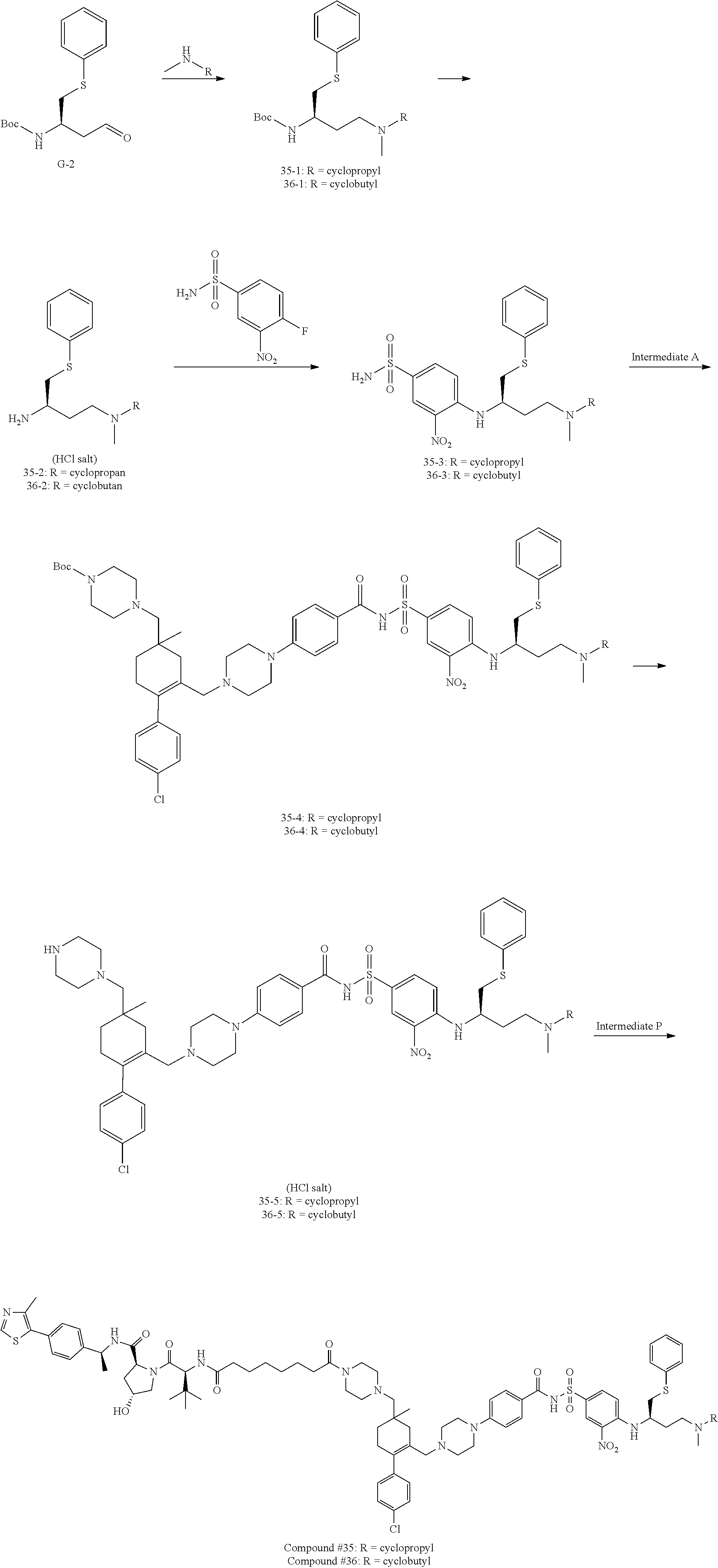

Compounds #35-36 were prepared by following General Procedure D. tert-butyl (R)-(4-(cyclopropyl(methyl)amino)-1-(phenylthio)butan-2-yl)carbamate (35-1) (28 mg, 79% yield). ¹H NMR (600 MHz, CDCl₃) δ 7.42 (d, J=7.7 Hz, 2H), 7.34-7.26 (m, 2H), 7.19 (t, J=7.5 Hz, 1H), 5.52 (s, 1H), 3.83 (s, 1H), 3.25 (d, J=13.5 Hz, 1H), 3.09-2.99 (m, 1H), 2.74-2.59 (m, 2H), 2.36 (s, 3H), 1.96-1.84 (m, 1H), 1.75-1.63 (m, 2H), 1.45 (s, 9H), 0.57-0.43 (m, 4H) ppm. LC/MS (ESI) m/z 351.2; [M+H]⁺ calcd for C₁₉H₃₁N₂O₂S⁺: 351.21.

tert-butyl (R)-(4-(cyclobutyl(methyl)amino)-1-(phenylthio)butan-2-yl)carbamate (36−1) (32 mg, 86% yield). ¹H NMR (600 MHz, MeOD) δ 7.44 (d, J=7.7 Hz, 2H), 7.33 (td, J=7.7, 1.7 Hz, 2H), 7.26-7.20 (m, 1H), 3.74-3.63 (m, 2H), 3.19-2.96 (m, 4H), 2.68 (s, 3H), 2.38-2.20 (m, 3H), 2.17-2.08 (m, 1H), 2.00 (s, 3H), 1.91-1.75 (m, 2H), 1.46 (s, 9H) ppm. LC/MS (ESI) m/z 365.1; [M+H]⁺ calcd for C₂₀H₃₃N₂O₂S⁺: 365.23.

(R)-N1-cyclopropyl-N1-methyl-4-(phenylthio)butane-1,3-diamine hydrochloride (35-2) (25 mg, quantitative yield). LC/MS (ESI) m/z 251.2; [M+H]⁺ calcd for C₁₄H₂₃N₂S⁺: 251.16.

(R)-N1-cyclobutyl-N1-methyl-4-(phenylthio)butane-1,3-diamine hydrochloride (36-2) (34 mg, quantitative yield). LC/MS (ESI) m/z 265.1; [M+H]⁺ calcd for C₁₅H₂₅N₂S⁺: 265.17.

(R)-4-((4-(cyclopropyl(methyl)amino)-1-(phenylthio)butan-2-yl)amino)-3-nitrobenzenesulfonamide (35-3) (14 mg, 39% yield in two steps). ¹H NMR (600 MHz, CDCl₃) δ 8.70-8.65 (m, 1H), 7.71 (dd, J=9.1, 2.3 Hz, 1H), 7.40-7.33 (m, 2H), 7.33-7.23 (m, 3H), 6.63 (d, J=9.2 Hz, 1H), 3.92 (p, J=6.1 Hz, 1H), 3.18-3.07 (m, 2H), 2.79-2.70 (m, 1H), 2.61-2.53 (m, 1H), 2.33 (s, 3H), 2.16-2.08 (m, 1H), 1.94-1.82 (m, 1H), 1.63 (p, J=5.8 Hz, 1H), 0.51-0.38 (m, 3H), 0.28-0.20 (m, 1H) ppm. LC/MS (ESI) m/z 451.1; [M+H]⁺ calcd for C₂₀H27N₄O₄S₂⁺: 451.15.

(R)-4-((4-(cyclobutyl(methyl)amino)-1-(phenylthio)butan-2-yl)amino)-3-nitrobenzenesulfonamide (36−3): (30 mg, 74% yield for two steps). ¹H NMR (600 MHz, Chloroform-d) δ 8.92 (d, J=8.3 Hz, OH), 8.69 (d, J=2.3 Hz, 1H), 7.74 (dt, J=9.3, 1.7 Hz, 1H), 7.39 (dq, J=6.2, 1.2 Hz, 2H), 7.34-7.21 (m, 4H), 6.71 (dd, J=9.3, 1.6 Hz, 1H), 4.06-3.95 (m, 1H), 3.23-3.10 (m, 2H), 2.78 (p, J=8.0 Hz, 1H), 2.56-2.46 (m, 1H), 2.25 (dt, J=12.5, 5.6 Hz, 1H), 2.16-2.05 (m, 4H), 2.04-1.95 (m, 2H), 1.95-1.84 (m, 2H), 1.75 (p, J=9.8 Hz, 1H), 1.68-1.52 (m, 2H) ppm. LC/MS (ESI) m/z 465.0; [M+H]⁺ calcd for C₂₁H₂₉N₄O₄S₂⁺: 465.16.

tert-butyl 4-((4′-chloro-6-((4-(4-(((4-(((R)-4-(cyclopropyl(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (35-4) (30 mg, 91% yield). ¹H NMR (600 MHz, CDCl₃) δ 8.86-8.78 (m, 2H), 7.98 (dt, J=9.2, 2.4 Hz, 1H), 7.71-7.63 (m, 2H), 7.41-7.34 (m, 2H), 7.33-7.20 (m, 6H), 7.02-6.98 (m, 2H), 6.81-6.75 (m, 2H), 6.62 (d, J=9.1 Hz, 1H), 3.98-3.88 (m, 1H), 3.42 (t, J=5.2 Hz, 4H), 3.28 (t, J=5.2 Hz, 4H), 3.18-3.08 (m, 2H), 2.86-2.73 (m, 3H), 2.63-2.45 (m, 5H), 2.44-2.27 (m, 8H), 2.27-2.19 (m, 2H), 2.18-2.08 (m, 2H), 1.99-1.85 (m, 2H), 1.70-1.57 (m, 3H), 1.47 (s, 9H), 0.96 (s, 3H), 0.53-0.39 (m, 3H), 0.30 (s, 1H) ppm. LC/MS (ESI) m/z 1055.9; [M+H]⁺ calcd for C₅₅H₇₂ClN₈O₇S₂⁺: 1055.46.

tert-butyl 4-((4′-chloro-6-((4-(4-(((4-(((R)-4-(cyclobutyl(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (36−4) (31 mg, 90% yield). ¹H NMR (600 MHz, Chloroform-d) δ 8.70 (s, 1H), 8.54 (s, 1H), 7.83 (t, J=10.8 Hz, 3H), 7.34-7.26 (m, 4H), 7.24-7.14 (m, 3H), 7.04-6.98 (m, 2H), 6.77 (d, J=8.5 Hz, 2H), 6.62 (d, J=9.2 Hz, 1H), 4.05-3.95 (m, 1H), 3.46-3.37 (m, 4H), 3.25 (t, J=5.2 Hz, 4H), 3.18-3.05 (m, 3H), 2.85 (s, 2H), 2.72 (s, 2H), 2.51 (d, J=5.0 Hz, 4H), 2.47-2.34 (m, 7H), 2.33-2.01 (m, 12H), 1.95 (d, J=17.3 Hz, 1H), 1.72 (q, J=10.2 Hz, 1H), 1.68-1.55 (m, 2H), 1.47 (s, 9H), 0.96 (s, 3H) ppm. LC/MS (ESI) m/z 1069.9; [M+H]⁺ calcd for C₅₆H₇₄ClN₈O₇S₂⁺: 1069.48.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(cyclopropyl(methyl)amino)-1-(phenylthio)butan-2-yl)amino)-3-nitrophenyl)sulfonyl)benzamide hydrochloride (35-5) (35 mg, quantitative yield). LC/MS (ESI) m/z 955.3; [M+H]⁺ calcd for C₅₀H₆₄ClN₈O₇S₂⁺: 955.41.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(cyclobutyl(methyl)amino)-1-(phenylthio)butan-2-yl)amino)-3-nitrophenyl)sulfonyl)benzamide hydrochloride (36-5) (37 mg, quantitative yield). LC/MS (ESI) m/z 969.4; [M+H]⁺ calcd for C₅₁H₆₆ClN₈O₇S₂⁺: 969.43.

(2S,4R)-1-((2S)-2-(8-(4-((4′-chloro-6-((4-(4-(((4-(((R)-4-(cyclopropyl(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #35) (17.9 mg, 41% yield in two steps). ¹H NMR (600 MHz, Chloroform-d) δ 8.77 (dd, J=3.8, 2.1 Hz, 1H), 8.68 (d, J=3.2 Hz, 1H), 7.97 (d, 1H), 7.76-7.68 (m, 2H), 7.46 (dd, 1H), 7.42-7.31 (m, 6H), 7.31-7.18 (m, 5H), 7.01-6.96 (m, 2H), 6.77 (d, J=8.6 Hz, 2H), 6.62 (d, J=9.3 Hz, 1H), 6.53-6.41 (m, 1H), 5.16 24-5.04 (m, 1H), 4.70 (p, J=7.6, 7.1 Hz, 1H), 4.63 (d, J=9.2, 3.8 Hz, 1H), 4.50 (d, J=7.5 Hz, 1H), 4.19-4.04 (m, 2H), 3.90 (s, 1H), 3.64-3.51 (m, 3H), 3.43 (q, J=4.5, 3.7 Hz, 2H), 3.24 (t, J=5.3 Hz, 4H), 3.18-3.07 (m, 2H), 2.90-2.79 (m, 2H), 2.79-2.69 (m, 1H), 2.62-2.46 (m, 8H), 2.45-2.14 (m, 15H), 2.14-2.02 (m, 2H), 1.96-1.85 (m, 2H), 1.69-1.52 (m, 7H), 1.51-1.40 (m, 4H), 1.35-1.23 (m, 7H), 1.05 (s, 9H), 0.96 (s, 3H), 0.44 (dtd, J=16.5, 8.2, 7.2, 4.3 Hz, 3H), 0.28 (dt, J=8.9, 4.2 Hz, 1H) ppm. LC/MS (ESI) m/z 1537.5.; [M+H]⁺ calcd for C₈₁H₁₀₆ClN₁₂O₁₀S₃⁺: 1537.70.

(2S,4R)-1-((2S)-2-(8-(4-((4′-chloro-6-((4-(4-(((4-(((R)-4-(cyclobutyl(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #36) (19.9 mg, 44% yield in two steps). ¹H NMR (600 MHz, Chloroform-d) δ 8.72 (d, J=3.7 Hz, 1H), 8.68 (d, J=1.2 Hz, 1H), 7.85 (s, 1H), 7.80 (d, J=8.3 Hz, 2H), 7.53-7.44 (m, 1H), 7.44-7.35 (m, 4H), 7.35-7.30 (m, 2H), 7.31-7.26 (m, 2H), 7.26-7.16 (m, 3H), 7.02-6.97 (m, 2H), 6.77 (d, J=8.7 Hz, 2H), 6.64 (d, J=9.2 Hz, 1H), 6.52-6.41 (m, 1H), 5.09 (p, J=7.2 Hz, 1H), 4.71 (s, 1H), 4.62 (dd, J=9.1, 3.8 Hz, 1H), 4.49 (s, 1H), 4.08 (s, 1H), 4.03-3.94 (m, 1H), 3.63-3.53 (m, 3H), 3.43 (t, J=5.3 Hz, 2H), 3.23 (t, J=5.2 Hz, 4H), 3.18-3.08 (m, 2H), 2.96 (s, 1H), 2.87-2.79 (m, 2H), 2.66-2.46 (m, 10H), 2.45-2.34 (m, 4H), 2.34-2.15 (m, 10H), 2.15-1.87 (m, 7H), 1.72-1.54 (m, 9H), 1.51-1.40 (m, 4H), 1.36-1.23 (m, 7H), 1.05 (s, 9H), 0.96 (s, 3H) ppm. LC/MS (ESI) m/z 1551.9; [M+H]⁺ calcd for C₈₂H₁₀₈ClN₁₂O₁₀S₃⁺: 1551.72.

### Example 20: Preparation of Compounds #37-39

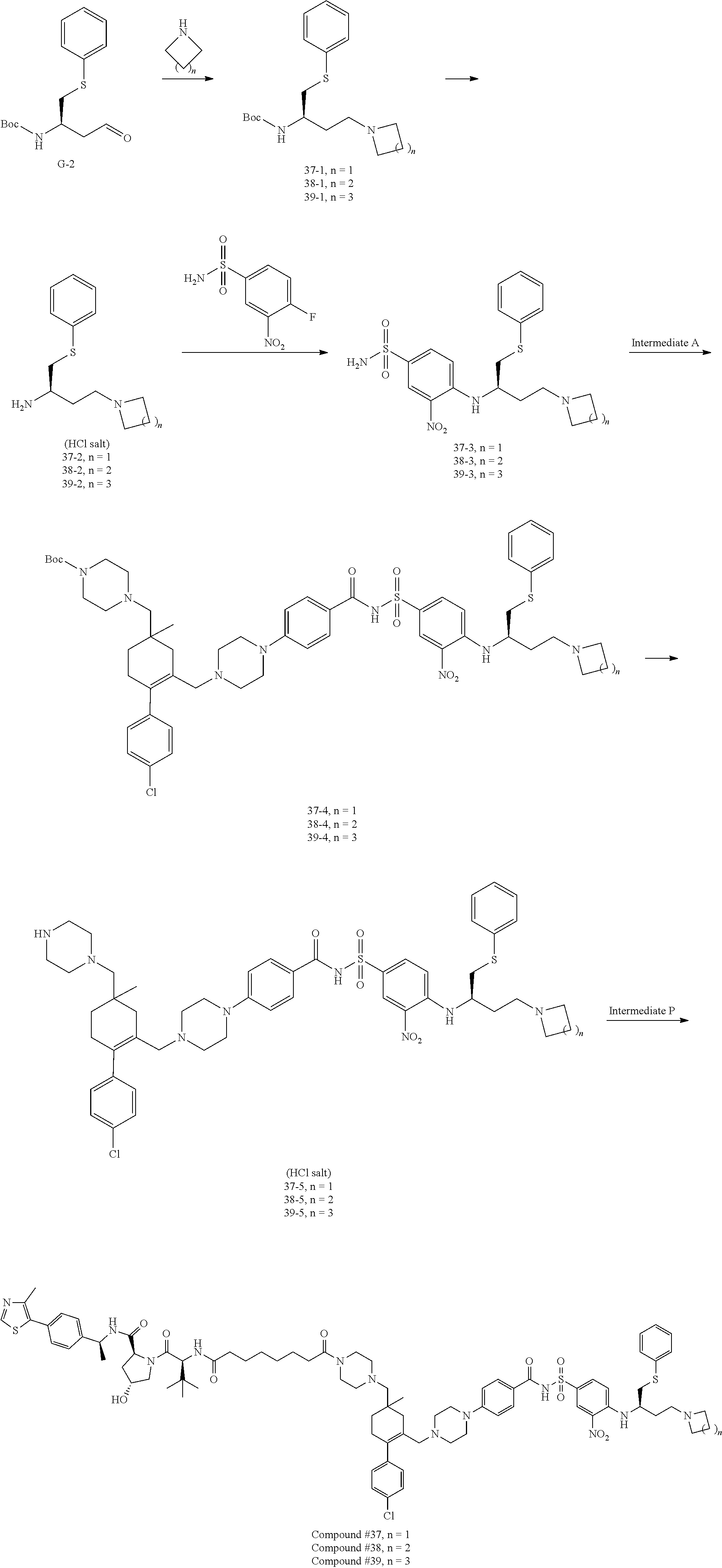

Compounds #37-39 were prepared by following General Procedure D.

tert-butyl (R)-(4-(azetidin-1-yl)-1-(phenylthio)butan-2-yl)carbamate (37-1) (21 mg, 61% yield). ¹H NMR (600 MHz, MeOD) δ 7.43 (d, J=7.7 Hz, 2H), 7.32 (t, J=7.8 Hz, 2H), 7.22 (t, J=7.3 Hz, 1H), 4.05 (t, J=8.1 Hz, 4H), 3.72-3.63 (m, 1H), 3.23-2.98 (m, 4H), 2.46 (p, J=8.1 Hz, 2H), 2.01-1.90 (m, 1H), 1.72-1.61 (m, 1H), 1.45 (s, 9H) ppm. LC/MS (ESI) m/z 337.2; [M+H]⁺ calcd for C₁₈H₂₉N₂O₂S⁺: 337.19.

tert-butyl (R)-(1-(phenylthio)-4-(pyrrolidin-1-yl)butan-2-yl)carbamate (38−1) (17 mg, 48% yield). ¹H NMR (600 MHz, CDCl₃) δ 7.41 (d, J=7.8 Hz, 2H), 7.33-7.27 (m, 2H), 7.23-7.17 (m, 1H), 5.49 (s, 1H), 3.76 (s, 1H), 3.21 (dd, J=13.9, 5.2 Hz, 1H), 3.15-2.92 (m, 6H), 2.85-2.74 (m, 1H), 2.15-2.05 (m, 1H), 2.01-1.97 (m, 5H), 1.44 (s, 9H) ppm. LC/MS (ESI) m/z 351.1; [M+H]⁺ calcd for C₁₉H₃₁N₂O₂S⁺: 351.21.

tert-butyl (R)-(1-(phenylthio)-4-(piperidin-1-yl)butan-2-yl)carbamate (39−1) (23 mg, 62% yield). ¹H NMR (600 MHz, Methanol-d₄) δ 7.44 (d, J=7.4 Hz, 2H), 7.33 (t, J=7.6 Hz, 2H), 7.23 (td, J=7.3, 1.3 Hz, 1H), 3.72-3.63 (m, 1H), 3.19-2.99 (m, 8H), 2.21-2.10 (m, 1H), 1.90-1.77 (m, 5H), 1.65 (t, J=6.0 Hz, 2H), 1.46 (s, 9H) ppm. LC/MS (ESI) m/z 365.2; [M+H]+ calcd for C₂₀H₃₃N₂O₂S⁺: 365.23.

(R)-4-(azetidin-1-yl)-1-(phenylthio)butan-2-amine hydrochloride (37-2) (20 mg, quantitative yield). LC/MS (ESI) m/z 237.0; [M+H]⁺ calcd for C₁₃H₂₁N₂S⁺: 237.14.

(R)-1-(phenylthio)-4-(pyrrolidin-1-yl)butan-2-amine hydrochloride (38-2) (15 mg, quantitative yield). LC/MS (ESI) m/z 251.0; [M+H]⁺ calcd for C₁₄H₂₃N₂S⁺: 251.16.

(R)-1-(phenylthio)-4-(piperidin-1-yl)butan-2-amine hydrochloride (39-2) (23 mg, quantitative yield). LC/MS (ESI) m/z 265.2; [M+H]⁺ calcd for C₁₅H₂₅N₂S⁺: 265.17.

(R)-4-((4-(azetidin-1-yl)-1-(phenylthio)butan-2-yl)amino)-3-nitrobenzenesulfonamide (37-3) (18 mg, 66% yield for two steps). ¹H NMR (600 MHz, MeOD) δ 8.55 (d, J=2.3 Hz, 1H), 7.79 (dd, J=9.1, 2.3 Hz, 1H), 7.36-7.31 (m, 2H), 7.24-7.16 (m, 3H), 6.99 (d, J=9.3 Hz, 1H), 4.13-4.06 (m, 1H), 3.43-3.34 (m, 6H), 3.22 (dd, J=14.4, 5.6 Hz, 1H), 2.74-2.63 (m, 2H), 2.18 (p, J=7.3 Hz, 2H), 2.00-1.91 (m, 1H), 1.88-1.79 (m, 1H) ppm. LC/MS (ESI) m/z 437.1; [M+H]⁺ calcd for C₁₉H₂₅N₄O₄S₂⁺: 437.13.

(R)-3-nitro-4-((1-(phenylthio)-4-(pyrrolidin-1-yl)butan-2-yl)amino)benzenesulfonamide (38-3) (16 mg, 73% yield for two steps). ¹H NMR (600 MHz, MeOD) δ 8.55 (t, J=2.4 Hz, 1H), 7.80 (dd, J=9.1, 2.3 Hz, 1H), 7.36-7.31 (m, 2H), 7.25-7.16 (m, 3H), 7.01 (dd, J=9.3, 4.4 Hz, 1H), 4.21-4.12 (m, 1H), 3.39 (dd, J=14.5, 5.4 Hz, 1H), 3.26 (dd, J=14.5, 5.7 Hz, 1H), 3.01-2.83 (m, 6H), 2.28-2.18 (m, 1H), 2.14-2.05 (m, 1H), 1.98-1.88 (m, 4H) ppm. LC/MS (ESI) m/z 451.0; [M+H]⁺ calcd for C₂₀H₂₇N₄O₄S₂⁺: 451.15.

(R)-3-nitro-4-((1-(phenylthio)-4-(piperidin-1-yl)butan-2-yl)amino)benzenesulfonamide (39-3) (20 mg, 68% yield for two steps). ¹H NMR (600 MHz, Methanol-d₄) δ 8.56 (d, J=2.3 Hz, 1H), 7.78 (dd, J=9.1, 2.3 Hz, 1H), 7.36−7.30 (m, 2H), 7.26-7.15 (m, 3H), 7.01 (d, J=9.2 Hz, 1H), 4.16-4.07 (m, 1H), 3.37 (dd, J=14.4, 5.4 Hz, 1H), 3.24 (dd, J=14.4, 5.9 Hz, 1H), 2.61-2.37 (m, 7H), 2.21-2.09 (m, 1H), 1.98-1.88 (m, 1H), 1.68-1.57 (m, 4H), 1.50 (d, J=8.3 Hz, 2H) ppm. LC/MS (ESI) m/z 465.1; [M+H]⁺ calcd for C₂₁H₂₉N₄O₄S₂⁺: 465.16.

tert-butyl 4-((6-((4-(4-(((4-(((R)-4-(azetidin-1-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (37-4) (24 mg, 48% yield). ¹H NMR (600 MHz, Chloroform-d) δ 8.55 (s, 1H), 7.92 (d, J=8.4 Hz, 2H), 7.83 (d, J=8.7 Hz, 1H), 7.33-7.20 (m, 4H), 7.19-7.08 (m, 3H), 7.00 (dt, J=8.3, 1.7 Hz, 2H), 6.78-6.63 (m, 3H), 4.17-3.81 (m, 5H), 3.40 (s, 4H), 3.25-3.01 (m, 9H), 2.82 (s, 2H), 2.56-2.26 (m, 12H), 2.25-2.17 (m, 3H), 2.14-1.89 (m, 4H), 1.46 (s, 9H), 0.96 (s, 3H) ppm. LC/MS (ESI) m/z 1041.1; [M+H]⁺ calcd for C₅₄H₇₀ClN₈O₇S₂⁺: 1041.45.

tert-butyl 4-((4′-chloro-4-methyl-6-((4-(4-(((3-nitro-4-(((R)-1-(phenylthio)-4-(pyrrolidin-1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (38-4) (13 mg, 51% yield), ¹H NMR (600 MHz, Chloroform-d) δ 8.62 (s, 1H), 8.28 (s, 1H), 7.95-7.79 (m, 3H), 7.32-7.24 (m, 4H), 7.22-7.10 (m, 3H), 7.01 (d, 2H), 6.75 (d, J=8.7 Hz, 3H), 4.13 (s, 1H), 3.41 (t, J=5.1 Hz, 4H), 3.28-3.03 (m, 11H), 2.83 (d, J=5.9 Hz, 2H), 2.58-2.45 (m, 6H), 2.44-2.26 (m, 5H), 2.26-1.91 (m, 11H), 1.61 (dt, J=14.4, 7.4 Hz, 1H), 1.47 (s, 9H), 0.96 (s, 3H) ppm. LC/MS (ESI) m/z 1055.8; [M+H]⁺ calcd for C₅₅H₇₂ClN₈O₇S₂⁺: 1055.46.

tert-butyl 4-((4′-chloro-4-methyl-6-((4-(4-(((3-nitro-4-(((R)-1-(phenylthio)-4-(piperidin-1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (39-4) (18 mg, 70% yield). ¹H NMR (600 MHz, Chloroform-d) δ 8.68 (s, 1H), 8.26 (s, 1H), 7.96-7.80 (m, 3H), 7.31-7.21 (m, 4H), 7.21-7.08 (m, 3H), 7.05-6.92 (m, 2H), 6.81-6.64 (m, 3H), 4.06 (s, 1H), 3.41 (t, J=5.0 Hz, 4H), 3.28-3.03 (m, 6H), 3.02-2.74 (m, 7H), 2.51 (s, 4H), 2.45-2.17 (m, 9H), 2.17-2.07 (m, 1H), 1.95 (d, J=17.4 Hz, 1H), 1.92-1.79 (m, 5H), 1.67-1.51 (m, 3H), 1.47 (s, 11H), 0.96 (s, 3H) ppm. LC/MS (ESI) m/z 1069.8; [M+H]⁺ calcd for C₅₆H₇₄ClN₈O₇S₂⁺: 1069.48.

N-((4-(((R)-4-(azetidin-1-yl)-1-(phenylthio)butan-2-yl)amino)-3-nitrophenyl)sulfonyl)-4-(4-((4′-chloro-4-methyl-4-(piperazin-1-ylmethyl)-3,4,5,6-tetrahydro-[1,1′-biphenyl]-2-yl)methyl)piperazin-1-yl)benzamide hydrochloride (37-5) (21 mg, 93% yield). LC/MS (ESI) m/z 941.4; [M+H]⁺ calcd for C₄₉H₆₂ClN₈O₅S₂⁺: 941.40.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((3-nitro-4-(((R)-1-(phenylthio)-4-(pyrrolidin-1-yl)butan-2-yl)amino)phenyl)sulfonyl)benzamide hydrochloride (38-5) (16 mg, quantitative yield). LC/MS (ESI) m/z 955.5; [M+H]⁺ calcd for C₅₀H₆₄ClN₈O₅S₂⁺: 955.41.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((3-nitro-4-(((R)-1-(phenylthio)-4-(piperidin-1-yl)butan-2-yl)amino)phenyl)sulfonyl)benzamide hydrochloride (39-5) (21 mg, quantitative yield). LC/MS (ESI) m/z 969.5; [M+H]⁺ calcd for C₅₁H₆₆ClN₈O₅S₂⁺: 969.43.

(2S,4R)-1-((2S)-2-(8-(4-((6-((4-(4-(((4-(((R)-4-(azetidin-1-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #37) (10.3 mg, 31% yield in two steps). ¹H NMR (600 MHz, Chloroform-d) δ 8.68 (s, 1H), 8.57 (s, 1H), 7.91 (d, J=8.4 Hz, 2H), 7.81 (d, 1H), 7.53 (dd, J=7.9, 4.1 Hz, 1H), 7.39 (q, J=8.4 Hz, 4H), 7.31-7.23 (m, 4H), 7.20-7.11 (m, 3H), 7.02-6.97 (m, 2H), 6.76 (d, J=8.6 Hz, 2H), 6.69 (d, J=9.2 Hz, 1H), 6.52-6.46 (m, 1H), 5.09 (p, J=7.0 Hz, 1H), 4.71 (t, J=8.1 Hz, 1H), 4.61 (d, J=9.0, 1.9 Hz, 1H), 4.49 (s, 1H), 4.06 (d, J=11.2 Hz, 2H), 3.87 (s, 4H), 3.65-3.51 (m, 2H), 3.46-3.37 (m, 2H), 3.19 (s, 4H), 3.14-3.00 (m, 4H), 2.85-2.76 (m, 2H), 2.61-2.45 (m, 7H), 2.44-2.32 (m, 8H), 2.32-2.08 (m, 11H), 2.08-1.88 (m, 3H), 1.64-1.53 (m, 6H), 1.52-1.40 (m, 4H), 1.35-1.23 (m, 5H), 1.04 (s, 9H), 0.95 (s, 3H) ppm. LC/MS (ESI) m/z 1523.8; [M+H]⁺ calcd for C₈₀H₁₀₄ClN₁₂O₁₀S₃⁺: 1523.68.

(2S,4R)-1-((2S)-2-(8-(4-((4′-chloro-4-methyl-6-((4-(4-(((3-nitro-4-(((R)-1-(phenylthio)-4-(pyrrolidin-1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #38) (10.7 mg, 57% yield in two steps). ¹H NMR (600 MHz, Chloroform-d) δ 8.67 (s, 1H), 8.62 (s, 1H), 7.86 (d, J=8.5 Hz, 2H), 7.81 (dd, J=9.0, 2.4 Hz, 1H), 7.53 (s, 1H), 7.38 (qd, J=8.3, 1.7 Hz, 4H), 7.31-7.23 (m, 4H), 7.21-7.12 (m, 3H), 6.99 (dd, J=8.3, 1.7 Hz, 2H), 6.75 (d, J=8.4 Hz, 3H), 6.60 (s, 1H), 5.12-5.02 (m, 1H), 4.66 (t, J=7.9 Hz, 1H), 4.60 (d, J=9.0 Hz, 1H), 4.47 (s, 1H), 4.13-3.97 (m, 2H), 3.61-3.50 (m, 3H), 3.48-3.37 (m, 2H), 3.23-2.96 (m, 12H), 2.81 (s, 2H), 2.60-2.46 (m, 6H), 2.41-2.08 (m, 16H), 2.07-1.85 (m, 7H), 1.65-1.53 (m, 6H), 1.51-1.40 (m, 4H), 1.35-1.22 (m, 5H), 1.03 (s, 9H), 0.95 (s, 3H) ppm. LC/MS (ESI) m/z 1537.5; [M+H]⁺ calcd for C₈₁H₁₀₆ClN₁₂O₁₀S₃⁺: 1537.70.

(2S,4R)-1-((2S)-2-(8-(4-((4′-chloro-4-methyl-6-((4-(4-(((3-nitro-4-(((R)-1-(phenylthio)-4-(piperidin-1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #39) (14.9 mg, 57% yield in two steps). ¹H NMR (600 MHz, Chloroform-d) δ 8.72−8.63 (m, 2H), 7.86 (dd, J=19.7, 8.7 Hz, 3H), 7.53 (d, J=8.7 Hz, 1H), 7.38 (qd, J=8.3, 1.6 Hz, 4H), 7.32-7.23 (m, 4H), 7.22-7.12 (m, 3H), 6.99 (dd, J=8.2, 1.5 Hz, 2H), 6.73 (t, J=9.7 Hz, 3H), 6.56 (d, J=8.6 Hz, 1H), 5.08 (p, J=7.1 Hz, 1H), 4.75-4.65 (m, 1H), 4.61 (d, 1H), 4.49 (d, J=4.1 Hz, 1H), 4.10-3.97 (m, 2H), 3.62-3.51 (m, 3H), 3.43 (dd, J=7.1, 3.5 Hz, 2H), 3.26-3.04 (m, 6H), 2.87-2.68 (m, 6H), 2.63-2.46 (m, 7H), 2.36 (s, 5H), 2.31-2.09 (m, 12H), 2.06-1.97 (m, 1H), 1.91 (d, J=17.4 24 Hz, 1H), 1.77 (s, 6H), 1.67-1.39 (m, 11H), 1.35-1.22 (m, 5H), 1.04 (s, 9H), 0.95 (s, 3H) ppm. LC/MS (ESI) m/z 1551.6; [M+H]⁺ calcd for C₈₁H₁₀₆ClN₁₂O₁₀S₃⁺: 1551.72.

### Example 21: Preparation of Compounds #41 and #43-44

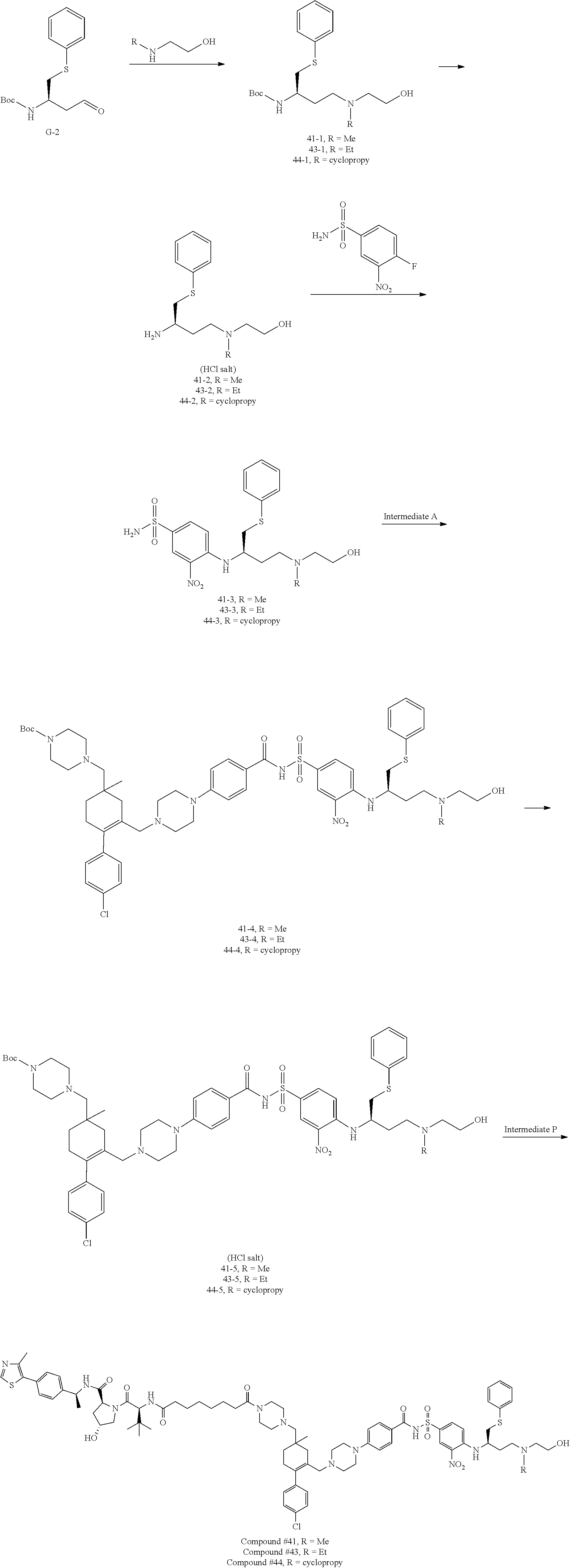

Compounds #41 and #43-44 were prepared by following General Procedure D. tert-butyl (R)-(4-((2-hydroxyethyl)(methyl)amino)-1-(phenylthio)butan-2-yl)carbamate (41-1) (27 mg, 75% yield). ¹H NMR (600 MHz, Methanol-d₄) δ 7.44 (d, J=7.4 Hz, 2H), 7.32 (t, J=7.8 Hz, 2H), 7.25-7.20 (m, 1H), 3.86-3.77 (m, 2H), 3.75-3.64 (m, 1H), 3.17-3.01 (m, 4H), 2.77 (s, 2H), 2.20-2.08 (m, 1H), 1.96 (s, 3H), 1.88-1.77 (m, 1H), 1.45 (s, 9H) ppm. LC/MS (ESI) m/z 355.2; [M+H]⁺ calcd for C₁₈H₃₁N₂O₃S⁺: 355.20.

tert-butyl (R)-(4-(ethyl(2-hydroxyethyl)amino)-1-(phenylthio)butan-2-yl)carbamate (43-1) (24 mg, 64% yield). ¹H NMR (600 MHz, Chloroform-d) δ 7.41 (d, J=7.7 Hz, 2H), 7.31 (t, J=7.6 Hz, 2H), 7.22 (t, J=7.4 Hz, 1H), 3.89-3.73 (m, 3H), 3.21 (dd, J=13.8, 5.3 Hz, 1H), 3.13-2.83 (m, 8H), 2.13-2.00 (m, 2H), 1.91 (d, J=13.0 Hz, 1H), 1.44 (s, 9H), 1.24 (t, J=7.3 Hz, 3H) ppm. LC/MS (ESI) m/z 369.2; [M+H]⁺ calcd for C₁₉H₃₃N₂O₃S⁺: 369.22.

tert-butyl (R)-(4-(cyclopropyl(2-hydroxyethyl)amino)-1-(phenylthio)butan-2-yl)carbamate (44-1) (25 mg, 65% yield). ¹H NMR (600 MHz, Methanol-d₄) δ 7.42 (d, J=7.8 Hz, 2H), 7.30 (t, J=7.6 Hz, 2H), 7.20 (t, J=7.4 Hz, 1H), 3.76-3.61 (m, 3H), 3.10 (dd, J=13.6, 6.4 Hz, 1H), 3.00 (dd, J=13.5, 6.6 Hz, 1H), 2.86-2.70 (m, 4H), 2.05-1.96 (m, 1H), 1.91-1.84 (m, 1H), 1.68-1.59 (m, 1H), 1.45 (s, 9H), 0.60-0.40 (m, 4H) ppm. LC/MS (ESI) m/z 381.1; [M+H]+ calcd for C₂₀H₃₃N₂O₃S⁺: 381.22.

(R)-2-((3-amino-4-(phenylthio)butyl)(methyl)amino)ethan-1-ol hydrochloride (41-2) (23 mg, quantitative yield). LC/MS (ESI) m/z 255.1; [M+H]⁺ calcd for C₁₃H₂₃N₂OS⁺: 255.15.

(R)-2-((3-amino-4-(phenylthio)butyl)(ethyl)amino)ethan-1-ol hydrochloride (43-2) (19 mg, 95% yield). LC/MS (ESI) m/z 269.0; [M+H]⁺ calcd for C₁₄H₂₅N₂OS⁺: 269.17.

(R)-2-((3-amino-4-(phenylthio)butyl)(cyclopropyl)amino)ethan-1-ol hydrochloride (44-2) (25 mg, quantitative yield). LC/MS (ESI) m/z 281.1; [M+H]⁺ calcd for C₁₄H₂₅N₂OS⁺: 281.17.

(R)-4-((4-((2-hydroxyethyl)(methyl)amino)-1-(phenylthio)butan-2-yl)amino)-3-nitrobenzenesulfonamide (41-3) (22 mg, 65% yield in two steps). ¹H NMR (600 MHz, Chloroform-d) δ 8.67-8.63 (m, 1H), 7.76-7.71 (m, 1H), 7.39-7.33 (m, 2H), 7.31-7.22 (m, 3H), 6.69 (d, J=9.2 Hz, 1H), 4.00 (dq, J=10.4, 5.7 Hz, 1H), 3.65-3.52 (m, 2H), 3.19-3.12 (m, 2H), 2.68-2.60 (m, 1H), 2.58-2.47 (m, 3H), 2.29 (s, 3H), 2.19-2.11 (m, 1H), 1.90-1.84 (m, 1H) ppm. LC/MS (ESI) m/z 455.0; [M+H]⁺ calcd for C₁₉H₂₇N₄O₅S₂⁺: 455.14.

(R)-4-((4-(ethyl(2-hydroxyethyl)amino)-1-(phenylthio)butan-2-yl)amino)-3-nitrobenzenesulfonamide (43−3) (24 mg, 83% yield). ¹H NMR (600 MHz, Chloroform-d) δ 8.64 (d, J=2.2 Hz, 1H), 7.74 (dd, J=9.1, 2.3 Hz, 1H), 7.36 (dd, J=7.7, 1.6 Hz, 2H), 7.32-7.19 (m, 3H), 6.70 (d, J=9.2 Hz, 1H), 4.04-3.93 (m, 1H), 3.61-3.52 (m, 2H), 3.15 (d, J=5.8 24 Hz, 2H), 2.75-2.57 (m, 7H), 2.25-2.07 (m, 2H), 1.88 (dq, J=14.2, 6.7 Hz, 1H), 1.04 (t, J=7.1 Hz, 3H) ppm. LC/MS (ESI) m/z 469.0; [M+H]⁺ calcd for C₂₀H₂₉N4O₅S₂⁺: 469.16.

(R)-4-((4-(cyclopropyl(2-hydroxyethyl)amino)-1-(phenylthio)butan-2-yl)amino)-3-nitrobenzenesulfonamide (44-3) (6 mg, 19% yield in two steps). ¹H NMR (600 MHz, Chloroform-d) δ 8.67 (d, J=2.3 Hz, 1H), 7.74 (dd, J=9.1, 2.3 Hz, 1H), 7.38-7.33 (m, 2H), 7.31-7.20 (m, 3H), 6.66 (d, J=9.2 Hz, 1H), 3.89 (td, J=7.7, 5.9, 3.3 Hz, 1H), 3.66-3.52 (m, 2H), 3.18-3.08 (m, 2H), 2.83-2.67 (m, 4H), 2.27-2.16 (m, 1H), 1.94-1.83 (m, 1H), 1.82-1.74 (m, 1H), 0.60-0.38 (m, 3H), 0.34-0.25 (m, 1H) ppm. LC/MS (ESI) m/z 481.0; [M+H]+ calcd for C₂₁H₂₉N₄O₅S₂⁺: 481.16.

tert-butyl 4-((4′-chloro-6-((4-(4-(((4-(((R)-4-((2-hydroxyethyl)(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (41-4) (22 mg, 43% yield). ¹H NMR (600 MHz, Chloroform-d) δ 8.67 (s, 1H), 7.91 (s, 1H), 7.83-7.73 (m, 2H), 7.37-7.12 (m, 7H), 7.00 (d, J=8.1 Hz, 2H), 6.78-6.70 (m, 2H), 6.67 (s, 1H), 4.02 (d, J=30.9 Hz, 1H), 3.76-3.66 (m, 3H), 3.41 (s, 4H), 3.23 (s, 4H), 3.14 (d, J=18.8 Hz, 2H), 2.93-2.69 (m, 8H), 2.62-2.26 (m, 11H), 2.26-2.09 (m, 4H), 2.03-1.88 (m, 2H), 1.60 (q, J=7.1 Hz, 1H), 1.53-1.43 (m, 9H), 0.95 (s, 3H) ppm. LC/MS (ESI) m/z 1059.1; [M+H]⁺ calcd for C₅₄H₇₂ClN₈O₈S₂⁺: 1059.46.

tert-butyl 4-((4′-chloro-6-((4-(4-(((4-(((R)-4-(ethyl(2-hydroxyethyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (43−4) (12 mg, 35% yield). ¹H NMR (600 MHz, Chloroform-d) δ 8.64 (s, 1H), 8.36 (s, 1H), 7.98-7.86 (m, 1H), 7.85-7.76 (m, 2H), 7.36-7.12 (m, 7H), 7.00 (d, J=8.4 Hz, 2H), 6.77-6.58 (m, 3H), 3.99 (s, 1H), 3.80-3.69 (m, 3H), 3.41 (s, 4H), 3.33-3.06 (m, 6H), 3.05-2.78 (m, 8H), 2.52 (d, J=18.3 Hz, 5H), 2.44-2.26 (m, 5H), 2.27-2.09 (m, 3H), 2.06-1.89 (m, 2H), 1.60 (td, J=14.8, 14.3, 7.2 Hz, 1H), 1.47 (s, 9H), 1.16-1.09 (m, 4H), 0.95 (s, 3H) ppm. LC/MS (ESI) m/z 1073.8; [M+H]⁺ calcd for C₅₅H₇₄ClN₈O₈S₂⁺: 1073.48.

tert-butyl 4-((4′-chloro-6-((4-(4-(((4-(((R)-4-(cyclopropyl(2-hydroxyethyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (44-4) (5.3 mg, 38% yield). ¹H NMR (600 MHz, Chloroform-d) δ 8.82 (d, J=2.3 24 Hz, 1H), 8.53 (d, J=8.5 Hz, 1H), 8.02 (dd, J=9.2, 2.3 Hz, 1H), 7.66 (d, J=9.0 Hz, 2H), 7.39-7.33 (m, 2H), 7.32-7.19 (m, 5H), 7.02-6.97 (m, 2H), 6.82-6.75 (m, 2H), 6.67 (d, J=9.3 Hz, 1H), 3.89 (d, J=5.4 Hz, OH), 3.63 (td, J=5.3, 4.8, 1.3 Hz, 2H), 3.42 (s, 5H), 3.29 (t, J=5.3 Hz, 4H), 3.23-3.10 (m, 2H), 2.87-2.71 (m, 6H), 2.51 (s, 5H), 2.44-2.11 (m, 10H), 1.98-1.88 (m, 2H), 1.83-1.76 (m, 1H), 1.66-1.57 (m, 1H), 1.47 (s, 10H), 0.96 (s, 3H), 0.60-0.42 (m, 3H), 0.39-0.29 (m, 1H) ppm. LC/MS (ESI) m/z 1085.9; [M+H]⁺ calcd for C₅₆H₇₄ClN₈O₈S₂⁺: 1085.48.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-((2-hydroxyethyl)(methyl)amino)-1-(phenylthio)butan-2-yl)amino)-3-nitrophenyl)sulfonyl)benzamide hydrochloride (41-5) (21 mg, quantitative yield). LC/MS (ESI) m/z 959.4; [M+H]⁺ calcd for C₄₉H₆₄ClN₈O₆S₂⁺: 959.41.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(ethyl(2-hydroxyethyl)amino)-1-(phenylthio)butan-2-yl)amino)-3-nitrophenyl)sulfonyl)benzamide hydrochloride (43-5) (14 mg, quantitative yield). LC/MS (ESI) m/z 973.4; [M+H]⁺ calcd for C₅₀H₆₆ClN₈O₆S₂⁺: 973.42.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(cyclopropyl(2-hydroxyethyl)amino)-1-(phenylthio)butan-2-yl)amino)-3-nitrophenyl)sulfonyl)benzamide hydrochloride (44-5) (8 mg, quantitative yield). LC/MS (ESI) m/z 985.4; [M+H]⁺ calcd for C₅₁H₆₆ClN₈O₆S₂⁺: 985.42.

(2S,4R)-1-((2S)-2-(8-(4-((4′-chloro-6-((4-(4-(((4-(((R)-4-((2-hydroxyethyl)(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #41) (4.4 mg, 14% yield in two steps). ¹H NMR (600 MHz, Chloroform-d) δ 8.74-8.65 (m, 2H), 7.92 (d, J=9.1 Hz, 1H), 7.78 (d, J=8.5 Hz, 2H), 7.51 (dd, J=12.6, 7.8 Hz, 1H), 7.38 (q, J=8.2 Hz, 5H), 7.34-7.25 (m, 4H), 7.24-7.15 (m, 3H), 7.02-6.97 (m, 2H), 6.75 (d, J=8.5 Hz, 2H), 6.68 (d, J=9.3 Hz, 1H), 6.53 (t, J=10.1 Hz, 1H), 5.09 (p, J=7.0 Hz, 1H), 4.71 (td, J=8.2, 4.1 Hz, 1H), 4.61 (dd, J=9.2, 3.5 Hz, 1H), 4.49 (s, 1H), 4.07 (d, J=11.2 Hz, 1H), 3.99 (s, 1H), 3.71-3.52 (m, 6H), 3.46-3.37 (m, 2H), 3.28-3.09 (m, 7H), 2.83 (t, J=9.7 Hz, 2H), 2.77-2.61 (m, 3H), 2.61-2.47 (m, 7H), 2.45-2.31 (m, 5H), 2.31-2.09 (m, 10H), 1.98-1.85 (m, 2H), 1.74-1.51 (m, 7H), 1.52-1.38 (m, 4H), 1.34-1.21 (m, 7H), 1.04 (s, 9H), 0.95 (s, 3H) ppm. LC/MS (ESI) m/z 1541.8; [M+H]+ calcd for C₈₀H₁₀₆ClN₁₂O₁₁S₃⁺: 1541.69.

(2S,4R)-1-((2S)-2-(8-(4-((4′-chloro-6-((4-(4-(((4-(((R)-4-(ethyl(2-hydroxyethyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #43) (4.14 mg, 24% yield in two steps). ¹H NMR (600 MHz, Chloroform-d) δ 8.71 (t, J=1.7 Hz, 1H), 8.68 (s, 1H), 7.92 (dd, J=9.2, 2.3 Hz, 1H), 7.79 (d, J=8.6 Hz, 2H), 7.52 (t, J=8.4 Hz, 1H), 7.38 (q, J=8.2 Hz, 5H), 7.34 34-7.25 (m, 4H), 7.23-7.15 (m, 3H), 7.02-6.96 (m, 2H), 6.76 (d, J=8.6 Hz, 2H), 6.68 (d, J=9.3 Hz, 1H), 6.60 (t, J=7.7 Hz, 1H), 5.08 (p, J=7.0 Hz, 1H), 4.68 (td, J=8.2, 2.7 Hz, 1H), 4.61 (dd, J=9.2, 2.1 Hz, 1H), 4.48 (s, 1H), 4.04 (d, J=11.2 Hz, 1H), 3.98 (s, 1H), 3.69-3.51 (m, 5H), 3.47-3.38 (m, 3H), 3.21 (t, J=5.3 Hz, 4H), 3.13 (d, J=5.6 Hz, 2H), 2.87-2.70 (m, 7H), 2.61-2.47 (m, 7H), 2.43-2.08 (m, 16H), 1.98-1.87 (m, 1H), 1.65-1.52 (m, 6H), 1.52-1.40 (m, 4H), 1.36-1.22 (m, 7H), 1.08 (t, J=7.1 Hz, 3H), 1.03 (s, 9H), 0.95 (s, 3H) ppm. LC/MS (ESI) m/z 1555.5; [M+H]⁺ calcd for C₈₁H₁₀₈ClN₁₂O₁₁S₃⁺: 1555.71.

(2S,4R)-1-((2S)-2-(8-(4-((4′-chloro-6-((4-(4-(((4-(((R)-4-(cyclopropyl(2-hydroxyethyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #44) (1.46 mg, 19% yield in two steps). ¹H NMR (600 MHz, Chloroform-d) δ 8.79 (s, 1H), 8.69 (d, J=2.1 Hz, 1H), 8.00 (dd, J=9.1, 2.4 Hz, 1H), 7.71 (t, J=7.7 Hz, 2H), 7.51−7.32 (m, 7H), 7.28 (s, 5H), 7.00 (dd, J=8.3, 1.8 14 Hz, 2H), 6.78 (d, 2H), 6.67 (d, J=9.2 Hz, 1H), 6.39 (s, 1H), 5.11 (s, 1H), 4.74 (s, 1H), 4.63 (t, J=8.4 Hz, 1H), 4.51 (s, 1H), 4.13 (s, 1H), 3.89 (s, 1H), 3.64-3.50 (m, 5H), 3.48-3.38 (m, 2H), 3.24 (s, 4H), 3.18-3.08 (m, 2H), 2.88-2.68 (m, 6H), 2.65-2.42 (m, 7H), 2.42-2.06 (m, 14H), 1.97-1.84 (m, 2H), 1.81-1.54 (m, 8H), 1.53-1.40 (m, 4H), 1.38-1.22 (m, 7H), 1.06 (s, 9H), 0.97 (s, 3H), 0.58-0.38 (m, 3H), 0.34 (s, 1H) ppm. LC/MS (ESI) m/z 1567.4; [M+H]+ calcd for C₈₁H₁₀₈ClN₁₂O₁₁S₃⁺: 1567.71.

### Example 22: Preparation of Compounds #45−48

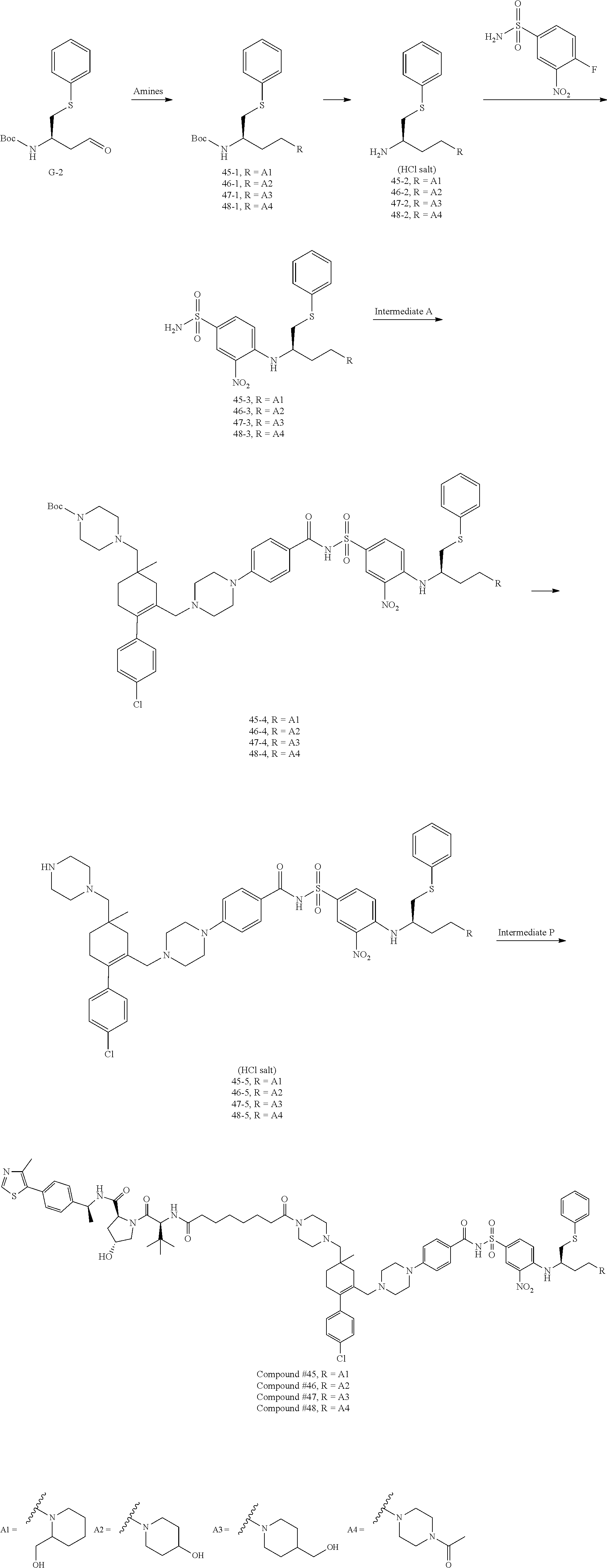

Compounds #45-48 were prepared following the General Procedure D. tert-butyl ((2R)-4-(2-(hydroxymethyl)piperidin-1-yl)-1-(phenylthio)butan-2-yl)carbamate (45-1) (26 mg, 65% yield). ¹H NMR (600 MHz, Methanol-d₄) δ 7.45-7.38 (m, 2H), 7.31 (t, J=7.7 Hz, 2H), 7.20 (t, J=7.4 Hz, 1H), 3.87-3.70 (m, 3H), 3.69-3.41 (m, 4H), 3.17-3.02 (m, 1H), 2.99 (dd, J=13.6, 7.0 Hz, 1H), 2.96-2.88 (m, 1H), 2.87-2.78 (m, 1H), 2.56-2.39 (m, 3H), 2.38-2.30 (m, 1H), 2.02-1.90 (m, 1H), 1.70-1.58 (m, 1H), 1.51-1.32 (m, 11H) ppm. LC/MS (ESI) m/z 395.1; [M+H]⁺ calcd for C₂₁H₃₅N₂O₃S⁺: 395.24.

tert-butyl (R)-(4-(4-hydroxypiperidin-1-yl)-1-(phenylthio)butan-2-yl)carbamate (46-1) (37 12 mg, 96% yield). ¹H NMR (600 MHz, Chloroform-d) δ 7.41 (d, J=7.7 Hz, 2H), 7.30 (t, 2H), 7.19 (t, J=7.4 Hz, 1H), 5.77 (s, 1H), 3.81 (s, 1H), 3.29-3.19 (m, 1H), 2.99 (dd, J=13.7, 7.2 Hz, 1H), 2.89 (t, J=9.2 Hz, 2H), 2.67-2.51 (m, 2H), 2.42 (s, 2H), 2.04-1.91 (m, 4H), 1.84-1.73 (m, 1H), 1.73-1.63 (m, 2H), 1.44 (s, 9H) ppm. LC/MS (ESI) m/z 382.3; [M+H]⁺ calcd for 2 C₂₀H₃₃N₂O₃S⁺: 381.22.

tert-butyl (R)-(4-(4-(hydroxymethyl)piperidin-1-yl)-1-(phenylthio)butan-2-yl)carbamate (47-1) (34 mg, 85% yield). ¹H NMR (600 MHz, Chloroform-d) δ 7.40 (d, J=7.7 Hz, 2H), 7.32-7.26 (m, 2H), 7.19 (t, J=7.4 Hz, 1H), 5.84 (s, 1H), 3.77 (s, 1H), 3.50 (d, J=5.9 Hz, 2H), 3.29-3.11 (m, 3H), 2.96 (dd, J=13.6, 7.3 Hz, 1H), 2.71 (d, J=11.3 Hz, 1H), 2.64-2.52 (m, 1H), 2.21 (q, J=11.8, 10.1 Hz, 2H), 2.05-1.96 (m, 1H), 1.88-1.74 (m, 3H), 1.65-1.55 (m, 1H), 1.54-1.38 (m, 11H) ppm. LC/MS (ESI) m/z 395.1; [M+H]⁺ calcd for C₂₁H₃₅N₂O₃S⁺: 395.24.

tert-butyl (R)-(4-(4-acetylpiperazin-1-yl)-1-(phenylthio)butan-2-yl)carbamate (48-1) (39 mg, 94% yield). ¹H NMR (600 MHz, Chloroform-d) δ 7.41 (d, J=7.7 Hz, 2H), 7.30 (t, J=7.8 Hz, 2H), 7.23-7.18 (m, 1H), 5.48 (s, 1H), 3.92 (s, 1H), 3.71-3.52 (m, 2H), 3.46 (t, J=5.1 Hz, 2H), 3.24 (d, J=13.3 Hz, 1H), 3.11-2.97 (m, 1H), 2.54-2.29 (m, 6H), 2.10 (s, 3H), 1.95-1.82 (m, 1H), 1.76-1.67 (m, 1H), 1.44 (s, 9H) ppm. LC/MS (ESI) m/z 408.2; [M+H]⁺ calcd for C₂₁H₃₄N₃O₃S⁺: 408.23.

(1-((R)-3-amino-4-(phenylthio)butyl)piperidin-2-yl)methanol hydrochloride (45-2) (28 mg, quantitative yield). LC/MS (ESI) m/z 295.2; [M+H]⁺ calcd for C₁₆H₂₇N₂OS⁺: 295.18.

(R)-1-(3-amino-4-(phenylthio)butyl)piperidin-4-ol hydrochloride (46−2): (17 mg, quantitative yield). LC/MS (ESI) m/z 281.1; [M+H]⁺ calcd for C₁₅H₂₅N₂OS⁺: 281.17.

(R)-(1-(3-amino-4-(phenylthio)butyl)piperidin-4-yl)methanol hydrochloride (47-2) (20 mg, quantitative yield). LC/MS (ESI) m/z 295.2; [M+H]⁺ calcd for C₁₆H₂₇N₂OS⁺: 295.18.

(R)-1-(4-(3-amino-4-(phenylthio)butyl)piperazin-1-yl)ethan-1-one hydrochloride (48−2) (20 28 mg, quantitative yield). LC/MS (ESI) m/z 308.2; [M+H]⁺ calcd for C₁₆H₂₆N₃OS⁺: 308.18.

4-(((2R)-4-(2-(hydroxymethyl)piperidin-1-yl)-1-(phenylthio)butan-2-yl)amino)-3-nitrobenzenesulfonamide (45-3) (23 mg, 71% yield). ¹H NMR (600 MHz, Methanol-d₄) δ 8.56 (d, J=2.3 Hz, 1H), 7.76 (dd, J=9.1, 2.3 Hz, 1H), 7.39-7.32 (m, 2H), 7.28-7.17 (m, 3H), 6.96 (d, J=9.3 Hz, 1H), 4.20-4.10 (m, 1H), 3.81-3.70 (m, 2H), 3.60-3.54 (m, 2H), 3.44-3.29 (m, 5H), 3.24 (dd, J=14.4, 5.8 Hz, 1H), 2.98 (ddd, J=13.1, 8.8, 6.7 Hz, 1H), 2.83 (dt, J=12.0, 4.2, 2.7 Hz, 1H), 2.48-2.34 (m, 2H), 2.31-2.23 (m, 1H), 2.20-2.12 (m, 1H), 1.95-1.85 (m, 1H) ppm. LC/MS (ESI) m/z 495.0; [M+H]⁺ calcd for C₂₂H₃₁N₄O₅S₂⁺: 495.17.

(R)-4-((4-(4-hydroxypiperidin-1-yl)-1-(phenylthio)butan-2-yl)amino)-3-nitrobenzenesulfonamide (46-3) (20 mg, 79% yield in two steps). ¹H NMR (600 MHz, Methanol-d₄) δ 8.56 (d, J=2.2 Hz, 1H), 7.78 (dd, J=9.2, 2.3 Hz, 1H), 7.37-7.31 (m, 2H), 7.26-7.17 (m, 3H), 7.01 (d, J=9.2 Hz, 1H), 4.17-4.09 (m, 1H), 3.65 (d, J=9.8 Hz, 1H), 3.36 (dd, J=14.4, 5.3 Hz, 1H), 3.24 (dd, J=14.4, 6.0 Hz, 1H), 2.89 (s, 1H), 2.82-2.73 (m, 1H), 2.59-2.44 (m, 2H), 2.33-2.07 (m, 4H), 1.98-1.79 (m, 4H), 1.64-1.49 (m, 2H) ppm. LC/MS (ESI) m/z 481.1; [M+H]⁺ calcd for C₂₁H₂₉N₄O₅S₂⁺: 481.16.

(R)-4-((4-(4-(hydroxymethyl)piperidin-1-yl)-1-(phenylthio)butan-2-yl)amino)-3-nitrobenzenesulfonamide (47-3) (18 mg, 87% yield in two steps). ¹H NMR (600 MHz, Methanol-d₄) δ 8.56 (d, J=2.3 Hz, 1H), 7.78 (dd, J=9.2, 2.3 Hz, 1H), 7.37-7.30 (m, 2H), 7.25-7.17 (m, 3H), 7.02 (d, J=9.2 Hz, 1H), 4.14 (tt, J=8.9, 4.5 Hz, 1H), 3.42 (d, J=6.4 Hz, 2H), 3.36 (dd, 1H), 3.25 (dd, J=14.4, 6.0 Hz, 1H), 3.11 (d, J=11.5 Hz, 1H), 3.02 (d, J=11.5 Hz, 1H), 2.66-2.55 (m, 2H), 2.24-2.11 (m, 2H), 2.02-1.92 (m, 1H), 1.85-1.72 (m, 2H), 1.60-1.48 (m, 1H), 1.37-1.26 (m, 3H) ppm. LC/MS (ESI) m/z 495.1; [M+H]⁺ calcd for C₂₂H₃₁N₄O₅S₂⁺: 495.17.

(R)-4-((4-(4-acetylpiperazin-1-yl)-1-(phenylthio)butan-2-yl)amino)-3-nitrobenzenesulfonamide (48-3) (18 mg, 85% yield in two steps). ¹H NMR (600 MHz, Methanol-d₄) δ 8.56 (d, J=2.3 Hz, 1H), 7.77 (dd, 1H), 7.38-7.32 (m, 2H), 7.28-7.18 (m, 3H), 7.01 (d, J=9.3 Hz, 1H), 4.24-4.15 (m, 1H), 3.63-3.49 (m, 4H), 3.36 (dd, 1H), 3.26 (dd, J=14.4, 6.0 Hz, 1H), 2.59-2.35 (m, 5H), 2.35-2.28 (m, 1H), 2.19-2.12 (m, 1H), 2.09 (s, 3H), 1.95-1.86 (m, 1H) ppm. LC/MS (ESI) m/z 508.1; [M+H]⁺ calcd for C₂₂H₃₀N₅O₅S₂⁺: 508.17.

tert-butyl 4-((4′-chloro-6-((4-(4-(((4-(((2R)-4-(2-(hydroxymethyl)piperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (45−4) (8 mg, 33% yield). ¹H NMR (600 MHz, Chloroform-d) δ 8.79 (s, 1H), 8.71-8.63 (m, 1H), 8.00 (d, J=9.4 Hz, 1H), 7.67 (s, 2H), 7.43-7.35 (m, 2H), 7.35-7.20 (m, 5H), 7.05-6.97 (m, 2H), 6.78 (s, 2H), 6.67-6.59 (m, 1H), 3.96 (s, 2H), 3.85-3.72 (m, 3H), 3.64-3.51 (m, 2H), 3.48-3.37 (m, 5H), 3.28 (t, J=5.2 Hz, 4H), 3.22-3.15 (m, 2H), 3.11 (dt, J=14.0, 6.8 Hz, 1H), 3.07-2.80 (m, 3H), 2.60-2.11 (m, 18H), 1.98-1.79 (m, 3H), 1.61 (dq, J=18.3, 10.6, 8.6 Hz, 1H), 1.47 (s, 9H), 0.96 (s, 3H) ppm. LC/MS (ESI) m/z 1099.5; [M+H]⁺ calcd for C₅₇H₇₆ClN₈O₈S₂⁺: 1099.49.

tert-butyl 4-((4′-chloro-6-((4-(4-(((4-(((R)-4-(4-hydroxypiperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (46−4) (12 mg, 46% yield). ¹H NMR (600 MHz, Chloroform-d) δ 8.68 (s, 1H), 8.44-8.27 (m, 1H), 7.86 (d, J=33.1 Hz, 3H), 7.35-7.11 (m, 6H), 7.00 (d, J=8.1 Hz, 2H), 6.79-6.64 (m, 3H), 3.99 (s, 1H), 3.90-3.79 (m, 1H), 3.41 (s, 4H), 3.22 (s, 4H), 3.03 (d, J=72.9 Hz, 3H), 2.89-2.66 (m, 4H), 2.50 (s, 5H), 2.46-2.27 (m, 6H), 2.27-2.09 (m, 6H), 2.08-1.85 (m, 5H), 1.81-1.56 (m, 3H), 1.47 (s, 9H), 0.96 (s, 3H) ppm. LC/MS (ESI) m/z 1085.6; [M+H]⁺ calcd for C₅₆H₇₄ClN₈O₈S₂⁺: 1085.48.

tert-butyl 4-((4′-chloro-6-((4-(4-(((4-(((R)-4-(4-(hydroxymethyl)piperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (47-4) (9 mg, 34% yield). ¹H NMR (600 MHz, Chloroform-d) δ 8.72 (s, 1H), 8.39 (s, 1H), 7.98-7.87 (m, 1H), 7.86-7.76 (m, 2H), 7.29 (t, J=7.5 Hz, 4H), 7.24-7.13 (m, 3H), 7.04-6.98 (m, 2H), 6.80-6.67 (m, 3H), 4.02 (s, 1H), 3.50-3.38 (m, 7H), 3.34-3.06 (m, 8H), 2.89-2.66 (m, 4H), 2.51 (s, 5H), 2.46-2.09 (m, 12H), 2.04-1.89 (m, 2H), 1.78 (s, 2H), 1.66-1.43 (m, 11H), 0.96 (d, J=2.1 Hz, 3H) ppm. LC/MS (ESI) m/z 1099.4; [M+H]⁺ calcd for C₅₇H₇₆ClN₈O₈S₂⁺: 1099.49.

tert-butyl 4-((6-((4-(4-(((4-(((R)-4-(4-acetylpiperazin-1-yl)-1-(phenylthio)butan-2-yl)amino)-2-(3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (48-4) (19 mg, 71% yield). ¹H NMR (600 MHz, Chloroform-d) δ 8.82 (q, J=2.8, 2.3 Hz, 1H), 8.70 (d, J=8.3 Hz, 1H), 8.02 (dt, J=9.2, 2.2 Hz, 1H), 7.74-7.63 (m, 2H), 7.40-7.32 (m, 2H), 7.32-7.20 (m, 5H), 7.05-6.94 (m, 2H), 6.82-6.76 (m, 2H), 6.73 (d, J=9.3 Hz, 1H), 4.02 (d, J=10.2 Hz, 1H), 3.73-3.65 (m, 1H), 3.57-3.52 (m, 1H), 3.47-3.44 (m, 2H), 3.41 (t, J=5.1 Hz, 5H), 3.28 (d, J=5.4 Hz, 5H), 3.22-3.09 (m, 2H), 2.84 (s, 2H), 2.60-2.26 (m, 13H), 2.26-2.11 (m, 5H), 2.09 (s, 3H), 1.94 (d, J=17.3 Hz, 1H), 1.90-1.80 (m, 1H), 1.66-1.56 (m, 1H), 1.47 (s, 9H), 0.96 (s, 3H) ppm. LC/MS (ESI) m/z 1112.6; [M+H]⁺ calcd for C₅₇H₇₅ClN₉O₈S₂⁺: 1112.49.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((2R)-4-(2-(hydroxymethyl)piperidin-1-yl)-1-(phenylthio)butan-2-yl)amino)-3-nitrophenyl)sulfonyl)benzamide hydrochloride (45-5) (6 2 mg, 80% yield). LC/MS (ESI) m/z 999.6; [M+H]⁺ calcd for C₅₂H₆₈ClN₈O₆S₂⁺: 999.44.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(4-hydroxypiperidin-1-yl)-1-(phenylthio)butan-2-yl)amino)-3-nitrophenyl)sulfonyl)benzamide hydrochloride (46-5) (16 mg, quantitative yield). LC/MS (ESI) m/z 985.4; [M+H]⁺ calcd for C₅₁H₆₆ClN₈O₆S₂⁺: 985.42.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(4-(hydroxymethyl)piperidin-1-yl)-1-(phenylthio)butan-2-yl)amino)-3-nitrophenyl)sulfonyl)benzamide hydrochloride (47-5) (11 12 mg, quantitative yield). LC/MS (ESI) m/z 999.5; [M+H]⁺ calcd for C₅₂H₆₈ClN₈O₆S₂⁺: 999.44.

N-((4-(((R)-4-(4-acetylpiperazin-1-yl)-1-(phenylthio)butan-2-yl)amino)-3-nitrophenyl)sulfonyl)-4-(4-((4′-chloro-4-methyl-4-(piperazin-1-ylmethyl)-3,4,5,6-tetrahydro-[1,1′-biphenyl]-2-yl)methyl)piperazin-1-yl)benzamide hydrochloride (48-5) (21 mg, quantitative yield). LC/MS (ESI) m/z 1012.5; [M+H]⁺ calcd for C₅₂H₆₇ClN₉O₆S₂⁺: 1012.43.

(2S,4R)-1-((2S)-2-(8-(4-((4′-chloro-6-((4-(4-(((4-(((2R)-4-(2-(hydroxymethyl)piperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #45) (7.2 mg, 79% yield). ¹H NMR (600 MHz, Chloroform-d) δ 8.74 (d, J=2.3 Hz, 1H), 8.67 (s, 1H), 7.95 (dd, J=9.2, 2.2 Hz, 1H), 7.72 (d, J=8.6 Hz, 2H), 7.54 (t, J=7.8 Hz, 1H), 7.42-7.30 (m, 7H), 7.30-7.17 (m, 5H), 7.00 26-6.94 (m, 2H), 6.76 (d, J=8.8 Hz, 2H), 6.67 (dd, J=10.6, 5.8 Hz, 2H), 5.07 (p, J=7.0 Hz, 1H), 4.64 (td, J=8.2, 2.3 Hz, 1H), 4.59 (dd, J=9.2, 2.4 Hz, 1H), 4.46 (s, 1H), 4.01 (d, J=11.4 Hz, 1H), 3.95 (s, 1H), 3.80-3.37 (m, 12H), 3.23 (t, J=5.3 Hz, 4H), 3.18-3.07 (m, 2H), 2.97-2.86 (m, 1H), 2.86-2.76 (m, 3H), 2.50 (s, 5H), 2.46-2.01 (m, 22H), 1.96-1.78 (m, 2H), 1.65-1.53 (m, 6H), 1.51-1.40 (m, 4H), 1.29 (d, J=6.5 Hz, 5H), 1.02 (s, 9H), 0.94 (s, 3H) ppm. LC/MS (ESI) m/z 1581.6; [M+H]⁺ calcd for C₈₃H₁₁₀ClN₁₂O₁₁S₃⁺: 1581.73.

(2S,4R)-1-((2S)-2-(8-(4-((4′-chloro-6-((4-(4-(((4-(((R)-4-(4-hydroxypiperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #46) (8.9 mg, 52% yield in two steps). ¹H NMR (600 MHz, Chloroform-d) δ 8.69 (d, J=2.1 Hz, 1H), 8.48-8.34 (m, 1H), 7.91 (s, 1H), 7.86-7.76 (m, 2H), 7.52 (d, J=15.9 Hz, 1H), 7.40 (q, J=8.6, 6.2 Hz, 4H), 7.33-7.25 (m, 3H), 7.25-7.13 (m, 4H), 7.01 (d, J=8.1 Hz, 2H), 6.75 (s, 3H), 6.34 (s, 1H), 5.11 (t, J=7.3 Hz, 1H), 4.75 (s, 1H), 4.61 (t, J=7.6 Hz, 1H), 4.52 (s, 1H), 4.18-4.07 (m, 1H), 4.02 (s, 1H), 3.82 (s, 1H), 3.63-3.52 (m, 2H), 3.42 (s, 2H), 3.28-3.06 (m, 7H), 3.00-2.79 (m, 2H), 2.76-2.48 (m, 7H), 2.48-2.08 (m, 22H), 2.04-1.84 (m, 5H), 1.72-1.52 (m, 8H), 1.50 (d, J=6.9 Hz, 3H), 1.46-1.39 (m, 1H), 1.36-1.21 (m, 5H), 1.06 (s, 9H), 0.96 (s, 3H) ppm. LC/MS (ESI) m/z 1567.5; [M+H]⁺ calcd for C₈₂H₁₀₈ClN₁₂O₁₁S₃⁺: 1567.71.

(2S,4R)-1-((2S)-2-(8-(4-((4′-chloro-6-((4-(4-(((4-(((R)-4-(4-(hydroxymethyl)piperidin-1-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #47) (5.4 mg, 42% yield in two steps). ¹H NMR (600 MHz, Chloroform-d) δ 8.77-8.59 (m, 2H), 8.32 (s, 1H), 7.95-7.76 (m, 3H), 7.57 (s, 1H), 7.39 (q, 4H), 7.33-7.21 (m, 4H), 7.20-7.11 (m, 2H), 7.01 (d, J=8.1 Hz, 2H), 6.73 (d, J=9.7 Hz, 3H), 6.42 (s, 1H), 5.10 (q, J=7.2 Hz, 1H), 4.75 (d, J=8.7 Hz, 1H), 4.61 (dd, J=9.1, 4.6 Hz, 1H), 4.51 (s, 1H), 4.13-3.95 (m, 2H), 3.64-3.52 (m, 3H), 3.42 (d, J=12.0 Hz, 3H), 3.34-3.00 (m, 8H), 2.98-2.72 (m, 3H), 2.62-2.46 (m, 8H), 2.45-2.07 (m, 20H), 2.00 (s, 1H), 1.90 (d, J=17.0 Hz, 1H), 1.77 (s, 2H), 1.70-1.38 (m, 12H), 1.35-1.20 (m, 5H), 1.05 (s, 9H), 0.96 (s, 3H) ppm. LC/MS (ESI) m/z 1582.0; [M+H]⁺ calcd for C₈₃H₁₁₀ClN₁₂O₁₁S₃⁺: 1581.73.

(2S,4R)-1-((2S)-2-(8-(4-((6-((4-(4-(((4-(((R)-4-(4-acetylpiperazin-1-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #48) (14.8 mg, 55% yield in two steps). ¹H NMR (600 MHz, Chloroform-d) δ 8.79 (p, J=2.3 Hz, 1H), 8.75-8.64 (m, 2H), 8.01 (dt, J=9.2, 2.6 Hz, 1H), 7.73 (t, J=7.9 Hz, 2H), 7.50 (dd, J=30.2, 7.9 Hz, 1H), 7.44-7.32 (m, 7H), 7.32-7.20 (m, 4H), 7.03-6.97 (m, 2H), 6.78 (d, 2H), 6.73 (d, J=9.3 Hz, 1H), 6.31 (d, J=9.5 Hz, 1H), 5.15-5.06 (m, 1H), 4.75 (q, J=8.6 Hz, 1H), 4.64 (dd, J=11.2, 8.8 Hz, 1H), 4.52 (s, 1H), 4.19-4.12 (m, 1H), 4.02 (s, 1H), 3.72-3.49 (m, 5H), 3.49-3.36 (m, 4H), 3.25 (t, J=5.3 Hz, 4H), 3.20-3.08 (m, 2H), 2.83 (d, J=17.0 Hz, 2H), 2.60 (s, 1H), 2.56-2.46 (m, 7H), 2.47-2.04 (m, 23H), 1.93-1.79 (m, 2H), 1.64-1.53 (m, 6H), 1.53-1.40 (m, 4H), 1.29 (d, J=11.5 Hz, 5H), 1.07 (s, 9H), 0.97 (s, 3H) ppm. LC/MS (ESI) m/z 1594.8; [M+H]⁺ calcd for C₈₃H₁₀₉ClN₁₃O₁₁S₃⁺: 1594.72.

### Example 23: Preparation of Compound #49

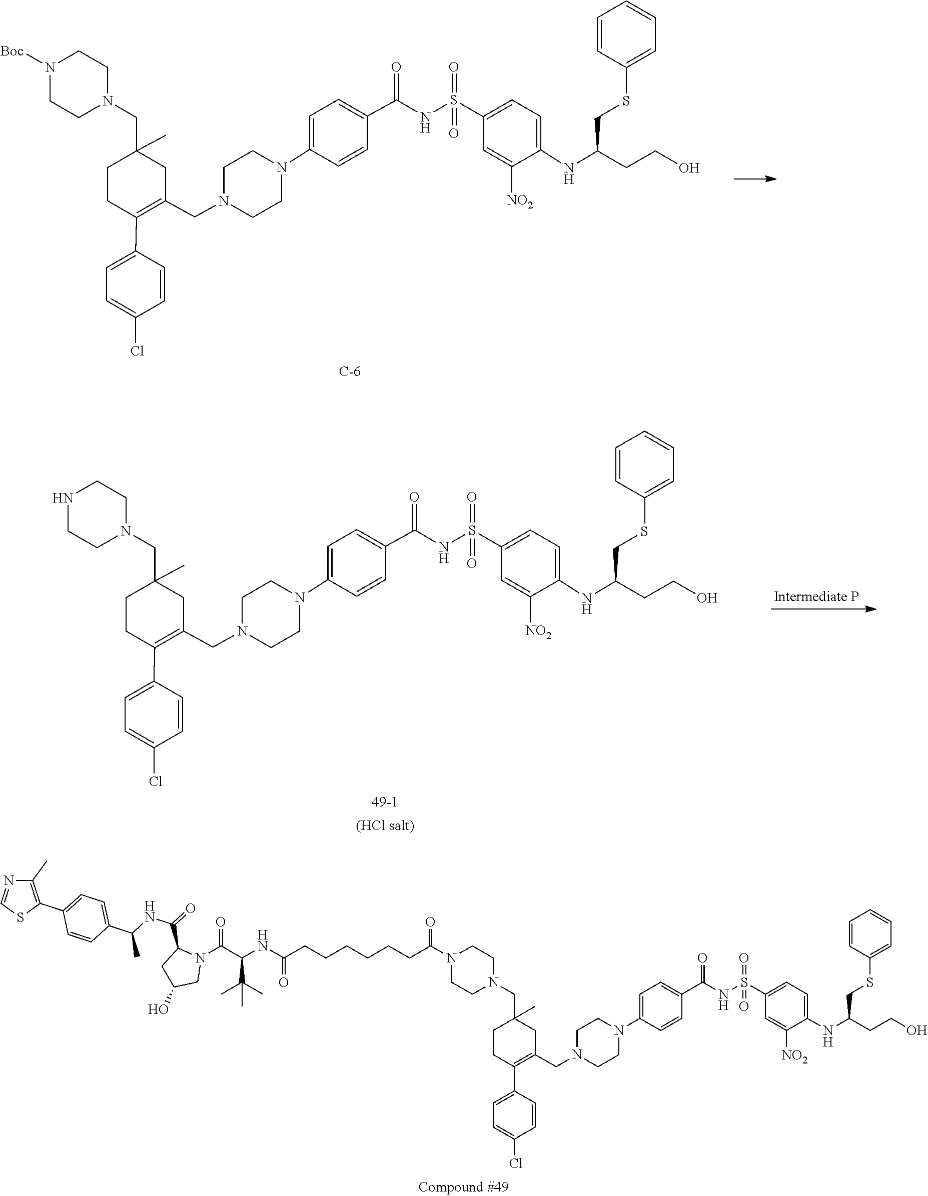

Preparation of 4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-hydroxy-1-(phenylthio)butan-2-yl)amino)-3-nitrophenyl)sulfonyl)benzamide hydrochloride (49-1): To solution of C-6 (6 mg, 0.006 mmol)) in DCM (0.5 mL) was added HCl dioxane solution (4N, 0.5 mL), then stirred at room temperature for 2 hours. The reaction mixture was concentrated and triturated with diethyl ether to afford crude 49-1 (5 mg, 89% yield) as an HCl salt, which was used directly in the next step. LC/MS (ESI) m/z 902.4; [M+H]⁺ calcd for C₄₆H₅₇ClN₇O₆S₂⁺: 902.35.

Preparation of (2S,4R)-1-((2S)-2-(8-(4-((4′-chloro-6-((4-(4-(((4-(((R)-4-hydroxy-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #49): General Procedure H was applied to afford compound #49 (2.7 mg, 34% yield). ¹H NMR (600 MHz, Chloroform-d) δ 8.79 (dd, J=6.0, 2.3 Hz, 1H), 8.70 (s, 1H), 8.62 (dd, J=8.5, 3.0 Hz, 1H), 7.98 (ddd, J=24.3, 9.2, 2.3 24 Hz, 1H), 7.72 (dd, J=12.3, 8.7 Hz, 2H), 7.49-7.33 (m, 7H), 7.32-7.17 (m, 5H), 7.04-6.97 (m, 2H), 6.79 (ddd, J=17.5, 9.4, 3.1 Hz, 3H), 6.31 (dd, J=28.5, 8.8 Hz, 1H), 5.11 (p, J=7.1 2 Hz, 1H), 4.76 (q, J=7.8 Hz, 1H), 4.63 (dd, J=9.0, 6.0 Hz, 1H), 4.52 (s, 1H), 4.15 (t, J=13.9 Hz, 2H), 3.84-3.71 (m, 1H), 3.68-3.50 (m, 3H), 3.42 (s, 2H), 3.32-3.13 (m, 6H), 2.87 (q, J=12.4 Hz, 2H), 2.61 (s, 1H), 2.56-2.39 (m, 8H), 2.40-2.31 (m, 2H), 2.31-2.04 (m, 11H), 1.98-1.83 (m, 2H), 1.69-1.40 (m, 11H), 1.37-1.20 (m, 5H), 1.06 (d, J=6.6 Hz, 9H), 0.97 (s, 3H) ppm. LC/MS (ESI) m/z 1484.7; [M+H]⁺ calcd for C₇₇H₉₉ClN₁₁O₁₁S₃⁺: 1484.64.

### Example 24: Preparation of Compound #50

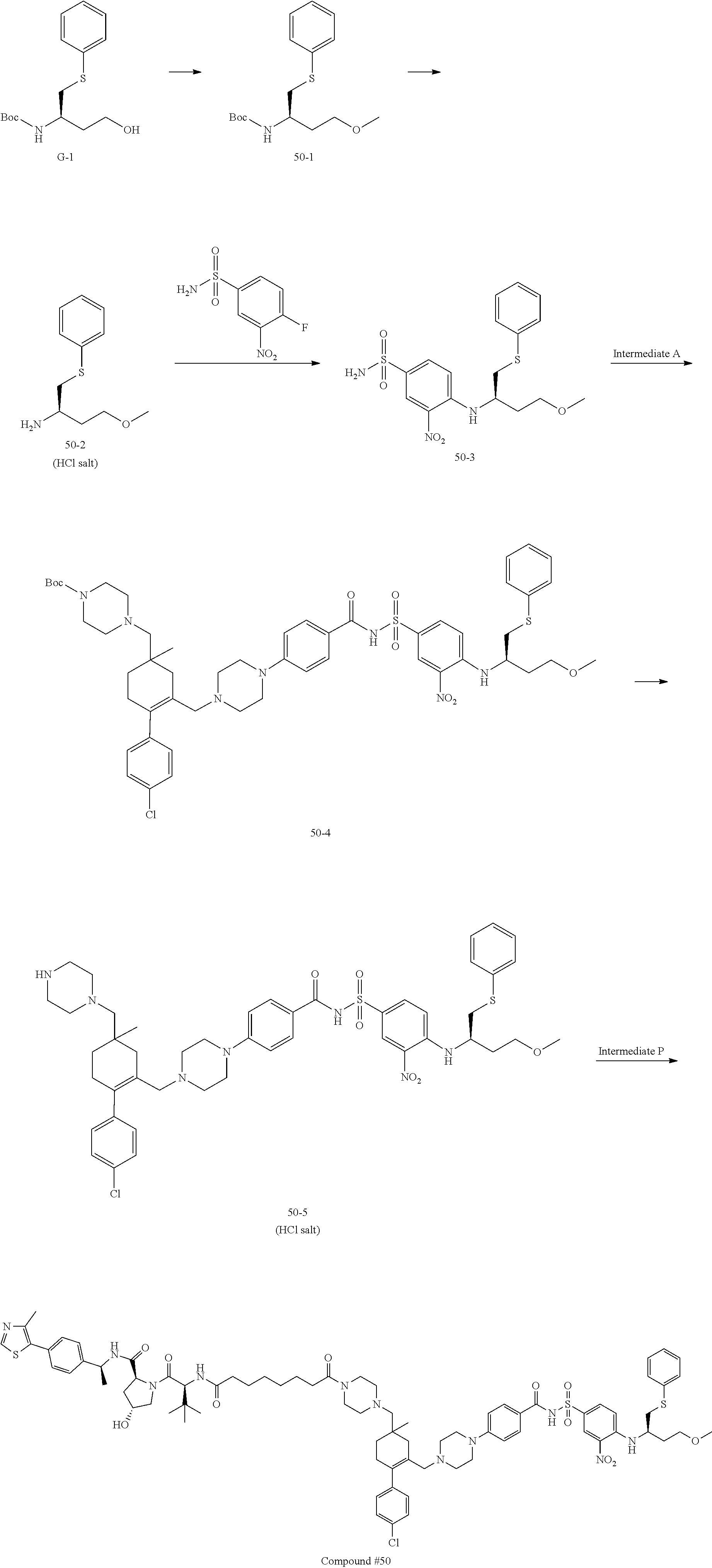

Preparation of tert-butyl (R)-(4-methoxy-1-(phenylthio)butan-2-yl)carbamate (50-1): A suspension of G-1 (50 mg, 0.17 mmol), iodomethane (120 mg, 0.84 mmol) and Ag₂O (59 mg, 0.25 mmol) in Et₂O (3 mL) was stirred at room temperature overnight. The resulting mixture was filtered, and the filtrate was concentrated. The residue was purified by flash column chromatography (0% to 50% of EtOAc in hexanes) to afford 50-1 (14 mg, 27% yield). ¹H NMR (600 MHz, Chloroform-d) δ 7.41 (d, J=7.7 Hz, 2H), 7.33-7.27 (m, 2H), 7.20 (t, J=7.6 Hz, 1H), 5.01 (s, 1H), 3.96 (s, 1H), 3.50-3.41 (m, 2H), 3.33 (s, 2H), 3.23 (dd, J=13.7, 4.9 Hz, 1H), 3.08 (s, 1H), 2.00-1.89 (m, 1H), 1.87-1.77 (m, 1H), 1.45 (s, 9H) ppm.

Preparation of (R)-4-methoxy-1-(phenylthio)butan-2-amine hydrochloride (50-2): To a solution of 50-1 (14 mg, 0.006 mmol)) in DCM (0.5 mL) was added 4N HCl in dioxane (0.5 mL), then stirred at room temperature for 2 hours. The reaction mixture was concentrated to afford crude 50-2 (12 mg, quantitative yield) as an HCl salt, which was used directly in the next step.

The last four steps for the preparation of compound #50 were based on General Procedure J.

(R)-4-((4-methoxy-1-(phenylthio)butan-2-yl)amino)-3-nitrobenzenesulfonamide (50-3) (15 mg, 87% yield in two steps). ¹H NMR (600 MHz, Chloroform-d) δ 8.72-8.66 (m, 2H), 7.74 (dd, J=9.2, 2.2 Hz, 1H), 7.37 (dq, J=5.9, 1.5 Hz, 2H), 7.32-7.21 (m, 3H), 6.72 (d, J=9.1 Hz, 1H), 4.13-4.01 (m, 1H), 3.51 (ddd, J=7.1, 4.3, 1.2 Hz, 2H), 3.34 (s, 3H), 3.24-3.10 (m, 2H), 2.23-2.12 (m, 1H), 2.02-1.93 (m, 1H) ppm.

tert-butyl 4-((4′-chloro-6-((4-(4-(((4-(((R)-4-methoxy-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (50-4) (18 mg, 73% yield). ¹H NMR (600 MHz, Chloroform-d) δ 8.83 (d, J=2.3 Hz, 1H), 8.78 (d, J=8.5 Hz, 1H), 8.03 (dd, J=9.3, 2.3 Hz, 1H), 7.70-7.64 (m, 2H), 7.38-7.34 (m, 2H), 7.32-7.20 (m, 5H), 7.03-6.98 (m, 2H), 6.82-6.77 (m, 2H), 6.72 (d, J=9.3 Hz, 1H), 4.12-4.02 (m, 1H), 3.52 (td, J=5.2, 4.5, 2.7 Hz, 2H), 3.42 (t, J=5.0 Hz, 4H), 3.34 (s, 3H), 3.28 (t, J=5.2 Hz, 4H), 3.22-3.13 (m, 2H), 2.83 (s, 2H), 2.51 (s, 4H), 2.43-2.26 (m, 5H), 2.26-2.10 (m, 5H), 2.03-1.97 (m, 1H), 1.94 (d, J=17.3 Hz, 1H), 1.68-1.57 (m, 1H), 1.47 (s, 10H), 0.96 (s, 3H) ppm. LC/MS (ESI) m/z 1016.3; [M+H]⁺ calcd for C₅₂H₆₇ClN₇O₈S₂⁺: 1016.42.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-methoxy-1-(phenylthio)butan-2-yl)amino)-3-nitrophenyl)sulfonyl)benzamide hydrochloride (50-5) (19 mg, quantitative yield). LC/MS (ESI) m/z 916.5; [M+H]⁺ calcd for C₄₇H₅₉ClN₇O₆S₂⁺: 916.37.

(2S,4R)-1-((2S)-2-(8-(4-((4′-chloro-6-((4-(4-(((4-(((R)-4-methoxy-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #50) (15.9 mg, 60% yield in two steps). ¹H NMR (600 MHz, Chloroform-d) δ 8.79 (q, J=2.1 Hz, 1H), 8.75 (t, J=6.3 Hz, 1H), 8.69 (d, J=1.9 Hz, 1H), 8.02 (d, J=9.3 Hz, 1H), 7.70 (dd, J=8.9, 3.6 Hz, 2H), 7.50-7.31 (m, 7H), 7.31-7.18 (m, 5H), 7.02-6.96 (m, 2H), 6.78 (d, 2H), 6.72 (d, 1H), 6.54-6.39 (m, 1H), 5.14-5.04 (m, 1H), 4.70 (d, J=9.8 Hz, 1H), 4.64 (dd, J=8.8, 5.3 Hz, 1H), 4.49 (d, J=4.9 Hz, 1H), 4.12-4.01 (m, 2H), 3.59 (dt, J=11.5, 3.4 Hz, 2H), 3.50 (t, J=7.0, 4.1, 1.8 Hz, 2H), 3.43 (t, J=4.8 Hz, 2H), 3.33 (s, 3H), 3.26 (d, J=5.3 Hz, 4H), 3.21-3.11 (m, 2H), 2.82 (s, 2H), 2.62-2.48 (m, 7H), 2.46-2.07 (m, 16H), 2.02-1.95 (m, 1H), 1.91 (dd, J=17.3, 8.5 Hz, 1H), 1.70-1.54 (m, 6H), 1.52-1.39 (m, 4H), 1.37-1.27 (m, 5H), 1.05 (s, 9H), 0.96 (s, 3H) ppm. LC/MS (ESI) m/z 1498.8; [M+H]+ calcd for C₇₈H₁₀₁ClN₁₁O₁₁S₃⁺: 1498.65.

### Example 25: Preparation of Compound #52

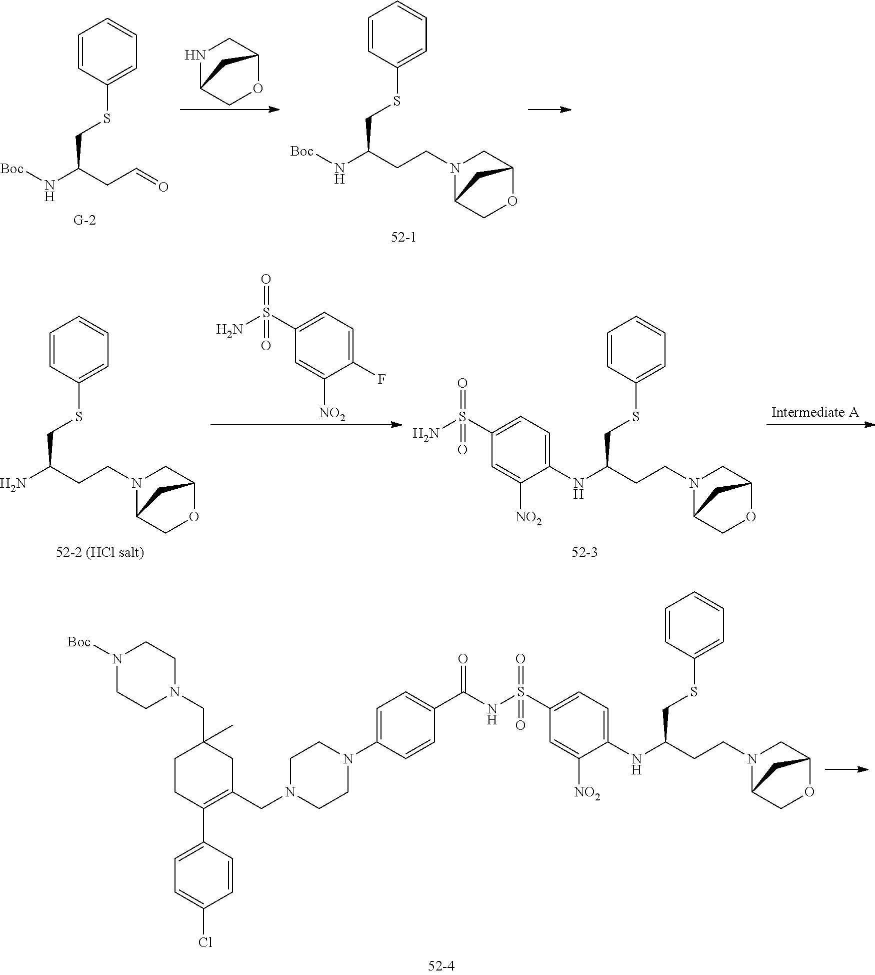

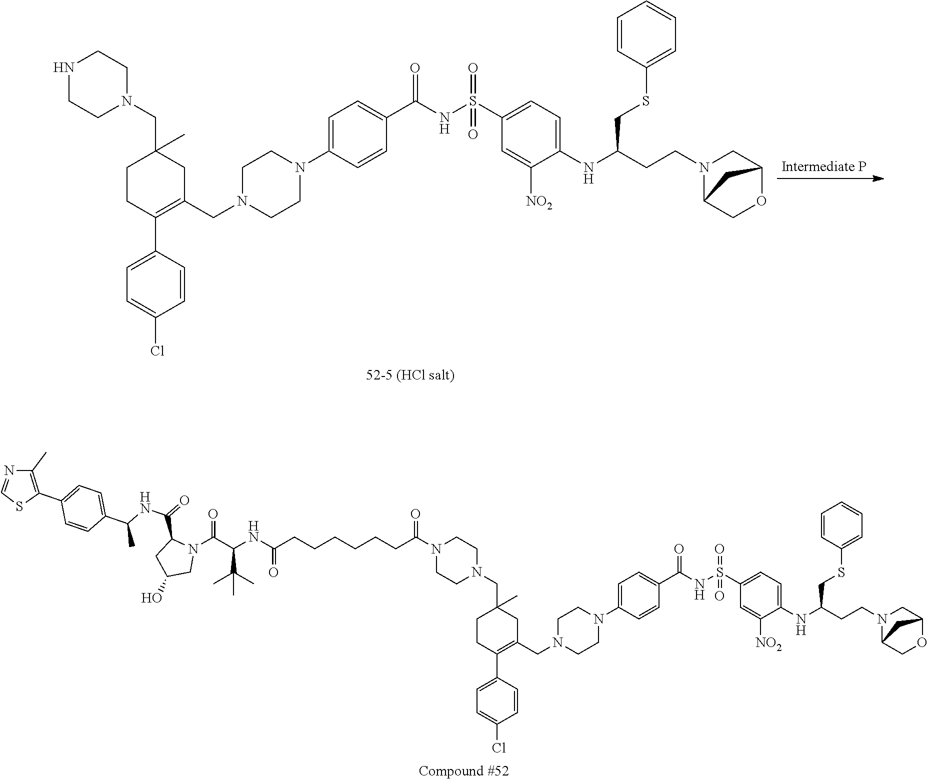 [SMILES: CC1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c([N+](=O)[O-])c4)cc3)CC2)C1 ; Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c([N+](=O)[O-])c6)cc5)CC4)C3)CC2)C(C)(C)C)cc1]

Compound #52 was prepared by following General Procedure D. tert-butyl ((R)-4-((1R,4R)-2-oxa-5-azabicyclo[2.2.1]heptan-5-yl)-1-(phenylthio)butan-2-yl)carbamate (52-1) (300 mg, 39% yield). ¹H NMR (600 MHz, Chloroform-d) δ 7.41 (d, 2H), 7.31 (dd, J=8.5, 7.0 Hz, 2H), 7.23-7.18 (m, 1H), 5.51 (s, 1H), 4.47 (s, 1H), 4.08 (d, J=8.5 Hz, 1H), 3.91-3.80 (m, 1H), 3.68 (dd, J=8.6, 1.6 Hz, 2H), 3.29-3.18 (m, 1H), 3.13-2.99 (m, 2H), 2.94-2.62 (m, 3H), 2.08-1.76 (m, 4H), 1.44 (s, 9H) ppm. LC/MS (ESI) m/z 379.0; [M+H]⁺ calcd for C₂₀H₃₁N₂O₃S⁺: 379.20.

(R)-4-((1R,4R)-2-oxa-5-azabicyclo[2.2.1]heptan-5-yl)-1-(phenylthio)butan-2-amine hydrochloride (52-2) (263 mg, quantitative yield). LC/MS (ESI) m/z 279.1; [M+H]⁺ calcd for C₁₅H₂₃N₂OS⁺: 279.15.

4-(((R)-4-((1R,4R)-2-oxa-5-azabicyclo[2.2.1]heptan-5-yl)-1-(phenylthio)butan-2-yl)amino)-3-nitrobenzenesulfonamide (52-3) (320 mg, 84% yield). ¹H NMR (600 MHz, Chloroform-d) δ 8.75-8.69 (m, 2H), 7.73 (dd, J=9.2, 2.3 Hz, 1H), 7.42-7.37 (m, 2H), 7.34-7.24 (m, 3H), 6.75 (d, J=9.2 Hz, 1H), 4.84 (s, 2H), 4.43 (t, J=2.1 Hz, 1H), 4.09 (t, J=10.0 Hz, 1H), 4.00 (d, J=7.8 Hz, 1H), 3.64 (dd, J=7.9, 1.8 Hz, 1H), 3.44 (s, 1H), 3.17 (d, J=6.0 Hz, 2H), 2.89 (d, J=9.9 Hz, 1H), 2.81-2.73 (m, 1H), 2.72-2.64 (m, 1H), 2.58 (d, J=10.0 Hz, 1H), 2.11-2.00 (m, 1H), 1.91-1.81 (m, 2H), 1.75 (d, J=9.8 Hz, 1H) ppm. LC/MS (ESI) m/z 479.0; [M+H]⁺ calcd for C₂₁H₂₇N₄O₅S₂⁺: 479.14.

tert-butyl 4-((6-((4-(4-(((4-(((R)-4-((1R,4R)-2-oxa-5-azabicyclo[2.2.1]heptan-5-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (52-4) (42 mg, 60% yield). ¹H NMR (600 MHz, Chloroform-d) δ 8.80 (d, J=2.3 Hz, 1H), 8.71 (d, J=8.4 Hz, 1H), 7.98 (dd, J=9.2, 2.4 Hz, 1H), 7.69 (d, J=8.6 Hz, 2H), 7.37-7.32 (m, 2H), 7.32-7.19 (m, 4H), 7.04-6.97 (m, 2H), 6.82-6.76 (m, 2H), 6.72 (d, J=9.3 Hz, 1H), 4.44 (d, J=2.2 Hz, 1H), 4.12-3.99 (m, 2H), 3.64 (dd, J=8.1, 1.7 Hz, 1H), 3.53 (s, 1H), 3.42 (t, J=5.0 Hz, 4H), 3.28 (t, J=5.2 Hz, 4H), 3.16 (d, J=5.8 Hz, 2H), 2.97 (d, J=10.1 Hz, 1H), 2.83 (s, 3H), 2.77-2.68 (m, 1H), 2.62 (d, J=10.1 Hz, 1H), 2.56-2.45 (m, 5H), 2.41-2.27 (m, 5H), 2.27-2.19 (m, 3H), 2.15 (d, J=17.4 Hz, 1H), 2.10-2.01 (m, 1H), 1.98-1.84 (m, 3H), 1.78 (d, J=10.2 Hz, 1H), 1.66-1.57 (m, 1H), 1.47 (s, 9H), 0.96 (s, 3H) ppm. LC/MS (ESI) m/z 1083.3; [M+H]⁺ calcd for C₅₆H₇₂ClN₈O₈S₂⁺: 1083.46.

N-((4-(((R)-4-((1R,4R)-2-oxa-5-azabicyclo[2.2.1]heptan-5-yl)-1-(phenylthio)butan-2-yl)amino)-3-nitrophenyl)sulfonyl)-4-(4-((4′-chloro-4-methyl-4-(piperazin-1-ylmethyl)-3,4,5,6-tetrahydro-[1,1′-biphenyl]-2-yl)methyl)piperazin-1-yl)benzamide hydrochloride (52-5) (37 mg, 94% yield). LC/MS (ESI) m/z 983.3; [M+H]⁺ calcd for C₅₁H₆₄ClN₈O₆S₂⁺: 983.41.

(2S,4R)-1-((2S)-2-(8-(4-((6-((4-(4-(((4-(((R)-4-((1R,4R)-2-oxa-5-azabicyclo[2.2.1]heptan-5-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #52) (17.8 mg, 73% yield). ¹H NMR (600 MHz, Chloroform-d) δ 8.80-8.56 (m, 3H), 7.95 (s, 1H), 7.81-7.70 (m, 2H), 7.53-7.36 (m, 4H), 7.37-7.16 (m, 6H), 7.01 (d, J=8.0 Hz, 2H), 6.81-6.67 (m, 3H), 6.34 (s, 1H), 5.15-5.05 (m, 1H), 4.75 (d, J=7.7 Hz, 1H), 4.63 (t, J=8.3 Hz, 1H), 4.52 (d, J=4.3 Hz, 1H), 4.44 (s, 1H), 4.14 (q, J=9.7, 8.3 Hz, 1H), 4.08-3.99 (m, 2H), 3.68-3.50 (m, 5H), 3.43 (s, 2H), 3.33-3.18 (m, 5H), 3.15 (s, 2H), 3.05-2.69 (m, 4H), 2.68-2.16 (m, 21H), 2.13-2.01 (m, 2H), 1.97 8-1.82 (m, 2H), 1.78 (s, 1H), 1.67-1.53 (m, 6H), 1.53-1.40 (m, 4H), 1.40-1.21 (m, 8H), 1.06 (s, 9H), 0.96 (s, 3H) ppm. LC/MS (ESI) m/z 1565.7; [M+H]⁺ calcd for C₈₂H₁₀₆ClN₁₂O₁₁S₃⁺: 1565.69.

### Example 26: Preparation of Compound #54

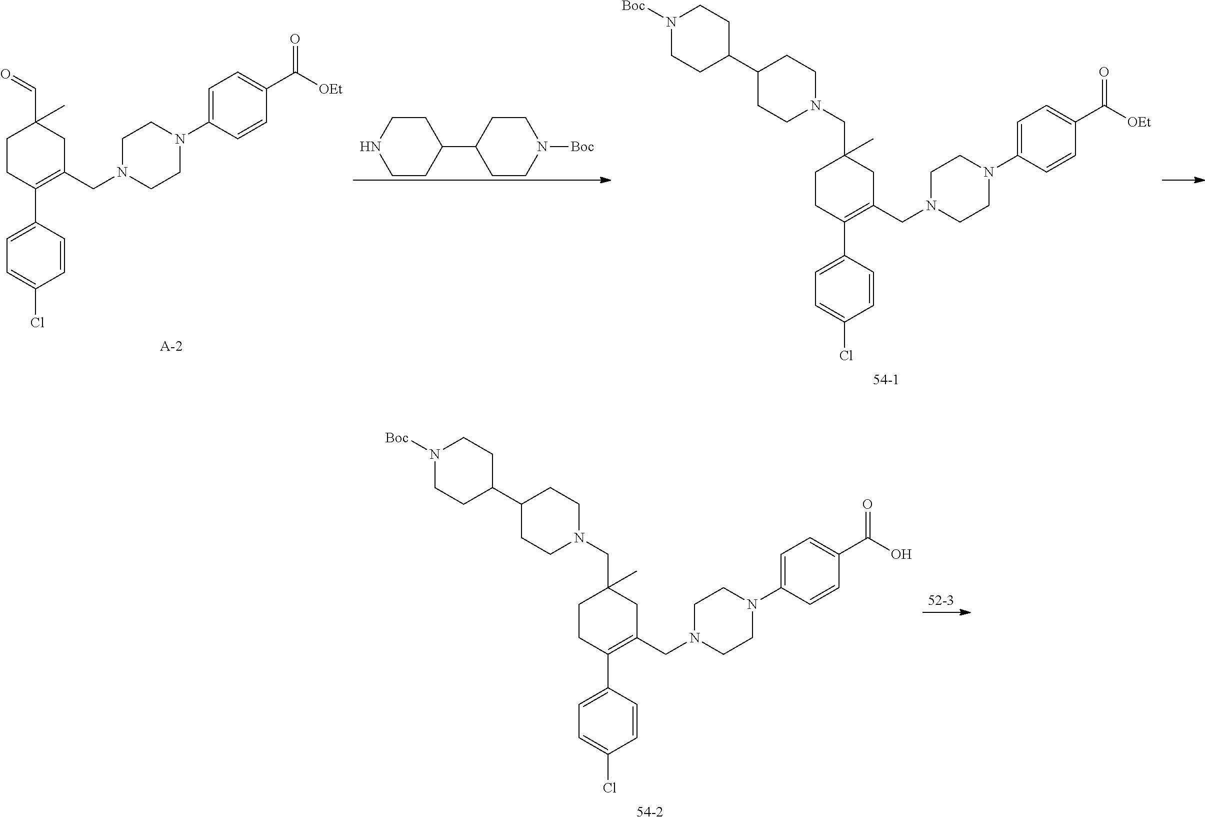

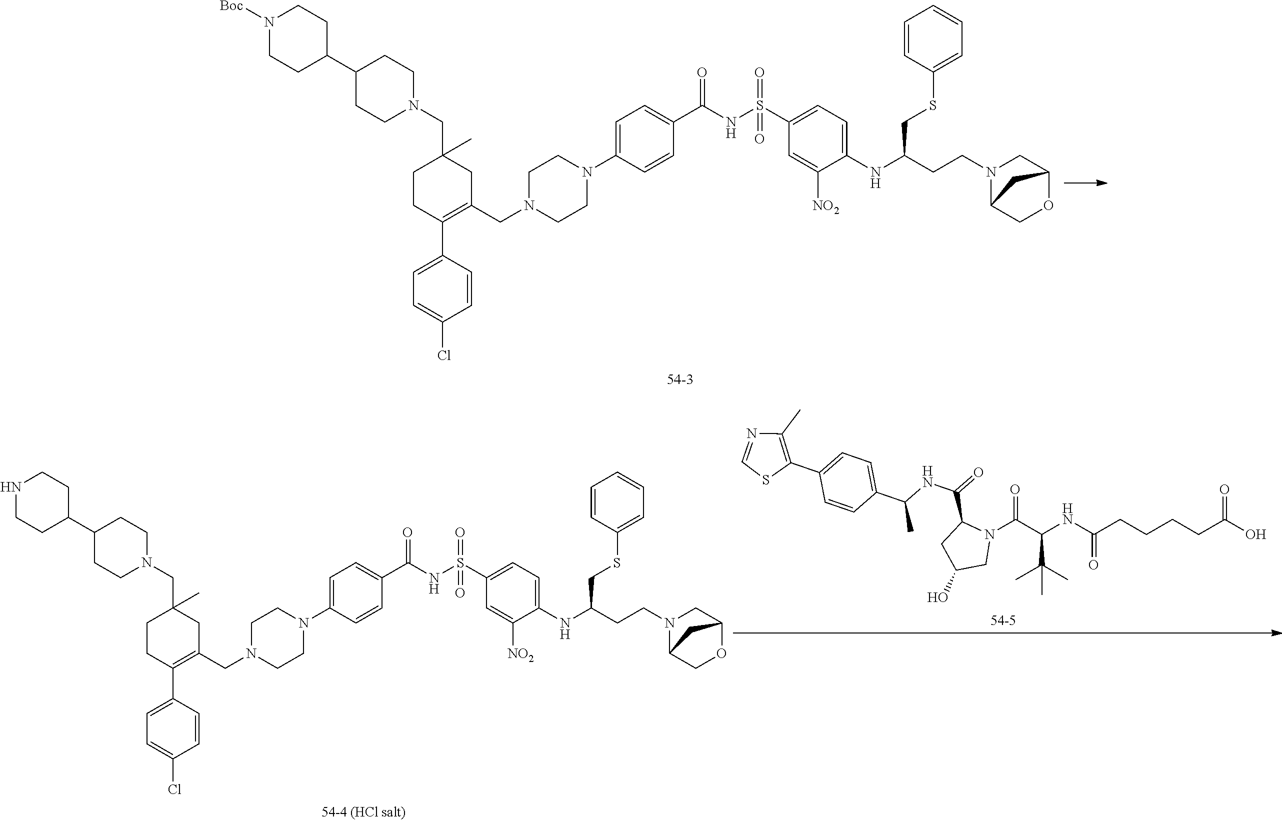 [SMILES: CC1(CN2CCC(C3CCN(C(=O)OC(C)(C)C)CC3)CC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c([N+](=O)[O-])c4)cc3)CC2)C1 ; CC1(CN2CCC(C3CCNCC3)CC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c([N+](=O)[O-])c4)cc3)CC2)C1 ; Cc1ncsc1-c1ccc([C@H](C)NC(=O)[C@@H]2C[C@@H](O)CN2C(=O)[C@@H](NC(=O)CCCCC(=O)O)C(C)(C)C)cc1]

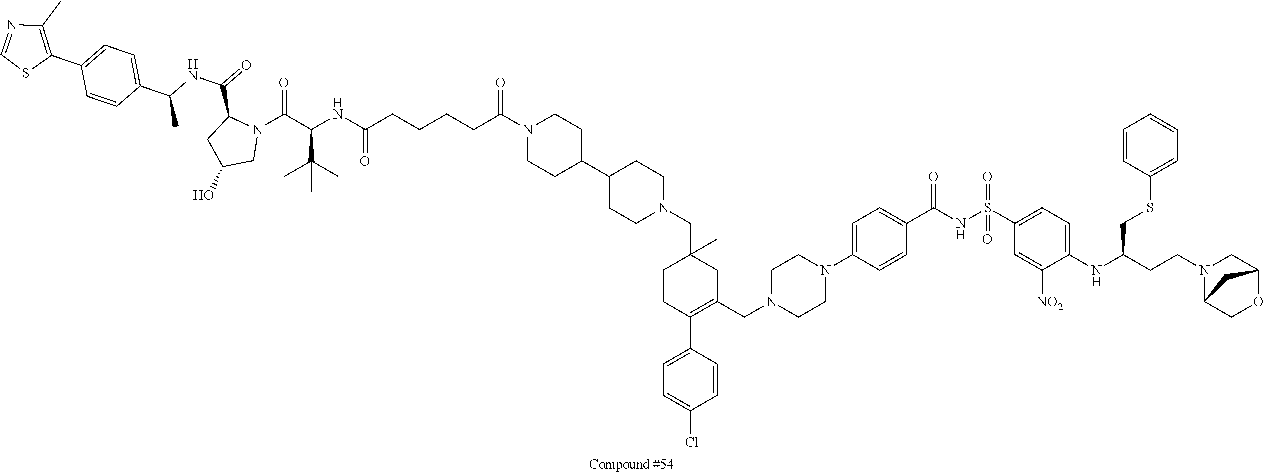 [SMILES: Cc1ncsc1-c1ccc([C@H](C)NC(=O)[C@@H]2C[C@@H](O)CN2C(=O)[C@@H](NC(=O)CCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c([N+](=O)[O-])c7)cc6)CC5)C4)CC3)CC2)C(C)(C)C)cc1]

Compound #54 was prepared by following the General Procedure F.

tert-butyl 1′-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-[4,4′-bipiperidine]-1-carboxylate (54-1) (99 18 mg, 81% yield). ¹H NMR (600 MHz, Chloroform-d) δ 7.94-7.88 (m, 2H), 7.29 (d, J=8.6 Hz, 2H), 7.04-6.99 (m, 2H), 6.86-6.81 (m, 2H), 4.34 (q, J=7.1 Hz, 2H), 4.13 (s, 2H), 3.27 (t, J=5.2 Hz, 4H), 2.86 (d, J=11.2 Hz, 2H), 2.82 (s, 2H), 2.65 (s, 2H), 2.44-2.33 (m, 4H), 2.32-2.11 (m, 7H), 1.92 (d, J=17.4 Hz, 1H), 1.68 (d, J=12.5 Hz, 2H), 1.65-1.55 (m, 3H), 1.51-1.42 (m, 10H), 1.38 (t, J=7.1 Hz, 3H), 1.34-1.25 (m, 2H), 1.25-1.09 (m, 3H), 1.06-0.99 (m, 1H), 0.95 (s, 3H) ppm. LC/MS (ESI) m/z 733.6; [M+H]⁺ calcd for C₄₃H₆₂ClN₄O₄⁺: 733.45.

4-(4-((4-((1′-(tert-butoxycarbonyl)-[4,4′-bipiperidin]-1-yl)methyl)-4′-chloro-4-methyl-3,4,5,6-tetrahydro-[1,1′-biphenyl]-2-yl)methyl)piperazin-1-yl)benzoic acid (54-2) (65 mg, 68% yield). LC/MS (ESI) m/z 705.4; [M+H]⁺ calcd for C₄₁H₅₈ClN₄O₄⁺: 705.41.

tert-butyl 1′-((6-((4-(4-(((4-(((R)-4-((1R,4R)-2-oxa-5-azabicyclo[2.2.1]heptan-5-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidine]-1-carboxylate (54-3) (32 mg, 64% yield). ¹H NMR (600 MHz, Chloroform-d) δ 8.79 (t, J=3.2 Hz, 1H), 8.75-8.53 (m, 1H), 7.95 (t, J=8.1 Hz, 1H), 7.88-7.74 (m, 1H), 7.36 (dt, J=8.5, 2.1 Hz, 2H), 7.32-7.19 (m, 10H), 7.04-6.97 (m, 2H), 6.77 (d, J=8.8 Hz, 2H), 6.71-6.57 (m, 1H), 4.44 (s, 1H), 4.19-3.94 (m, 5H), 3.68-3.60 (m, 1H), 3.52 (d, J=3.0 Hz, 1H), 3.30-3.02 (m, 8H), 2.96 (d, J=10.0 Hz, 1H), 2.92-2.76 (m, 2H), 2.76-2.54 (m, 3H), 2.51-2.18 (m, 10H), 2.13-2.00 (m, 2H), 1.92 (d, J=9.2 Hz, 1H), 1.88-1.82 (m, 1H), 1.78 (d, J=9.8 Hz, 1H), 1.72-1.60 (m, 5H), 1.48 (s, 9H), 1.37-1.16 (m, 6H), 1.16-0.98 (m, 2H) ppm. LC/MS (ESI) m/z 1165.4; [M+H]⁺ calcd for C₆₂H₈₂ClN₈O₈S₂⁺: 1165.54.

N-((4-(((R)-4-((1R,4R)-2-oxa-5-azabicyclo[2.2.1]heptan-5-yl)-1-(phenylthio)butan-2-yl)amino)-3-nitrophenyl)sulfonyl)-4-(4-((4-([4,4′-bipiperidin]-1-ylmethyl)-4′-chloro-4-methyl-3,4,5,6-tetrahydro-[1,1′-biphenyl]-2-yl)methyl)piperazin-1-yl)benzamide hydrochloride (54-5) (32 mg, quantitative yield). LC/MS (ESI) m/z 1065.5; [M+H]⁺ calcd for C₅₇H₇₄ClN₈O₆S₂⁺: 1065.49.

(2S,4R)-1-((2S)-2-(6-(1′-((6-((4-(4-(((4-(((R)-4-((1R,4R)-2-oxa-5-azabicyclo[2.2.1]heptan-5-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (compound #54) (12.9 mg, 58% yield in two steps). ¹H NMR (600 MHz, Chloroform-d) δ 8.80-8.74 (m, 1H), 8.68 (s, 1H), 7.93 (d, J=9.8 Hz, 1H), 7.86-7.80 (m, 2H), 7.51-7.31 (m, 7H), 7.31-7.18 (m, 4H), 7.00 (dd, J=6.5, 4.2 Hz, 2H), 6.74 (s, 2H), 6.65 (s, 2H), 5.08 (q, J=7.0 Hz, 1H), 4.73 (d, J=9.9 Hz, 1H), 4.65-4.52 (m, 1H), 4.49 (s, 1H), 4.42 (s, 1H), 4.09 (s, 1H), 4.01 (d, J=8.1 Hz, 2H), 3.82 (d, J=13.0 Hz, 1H), 3.66-3.57 (m, 2H), 3.49 (dd, J=6.8, 3.7 Hz, 1H), 3.28-3.05 (m, 8H), 3.00-2.82 (m, 3H), 2.77 (dt, J=13.4, 7.0 Hz, 1H), 2.72-2.64 (m, 1H), 2.60 (d, J=10.2 Hz, 1H), 2.55-2.18 (m, 21H), 2.16-1.99 (m, 3H), 1.92-1.79 (m, 2H), 1.78-1.54 (m, 10H), 1.53-1.40 (m, 4H), 1.39-1.22 (m, 10H), 1.16-0.96 (m, 12H) ppm. LC/MS (ESI) m/z 1619.9; [M+H]⁺ calcd for C₈₆H₁₁₂ClN₁₂O₁₁S₃⁺: 1619.74.

### Example 27: Preparation of Compounds #40, #42, #53, and #55-57

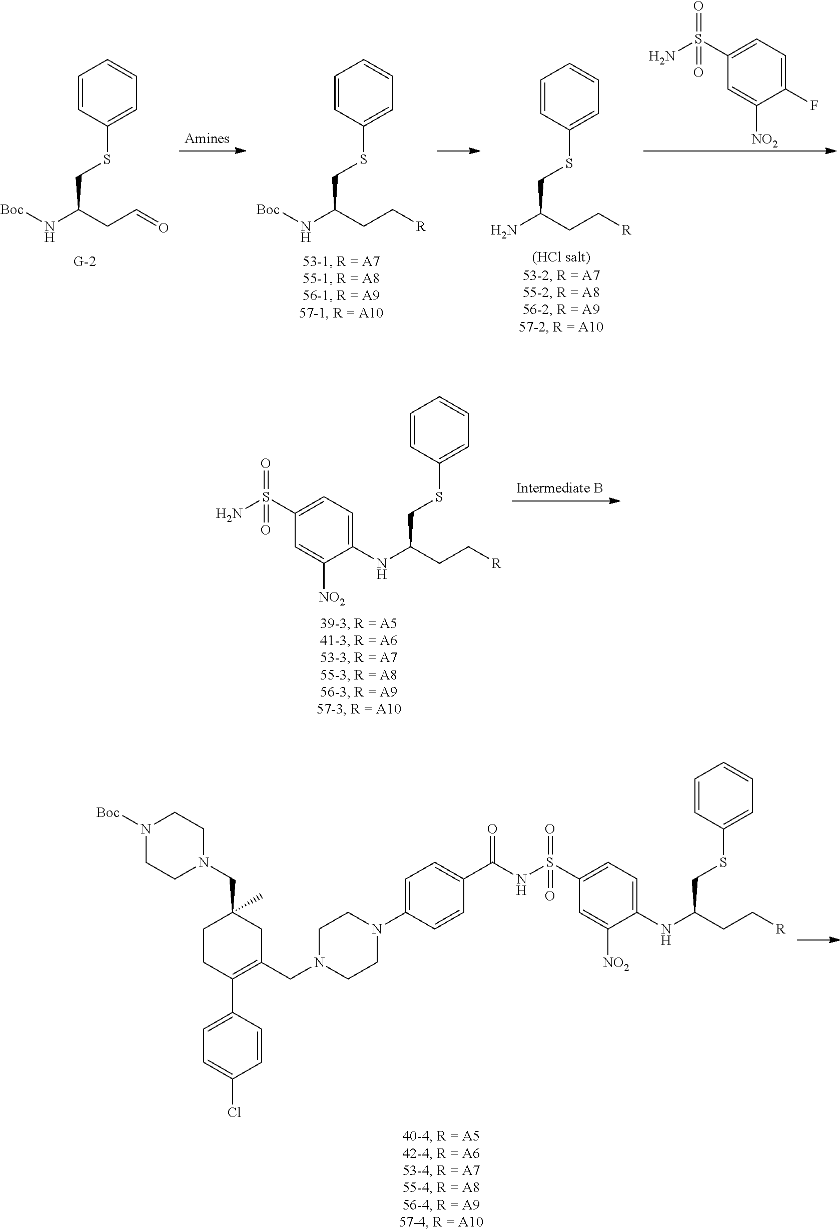

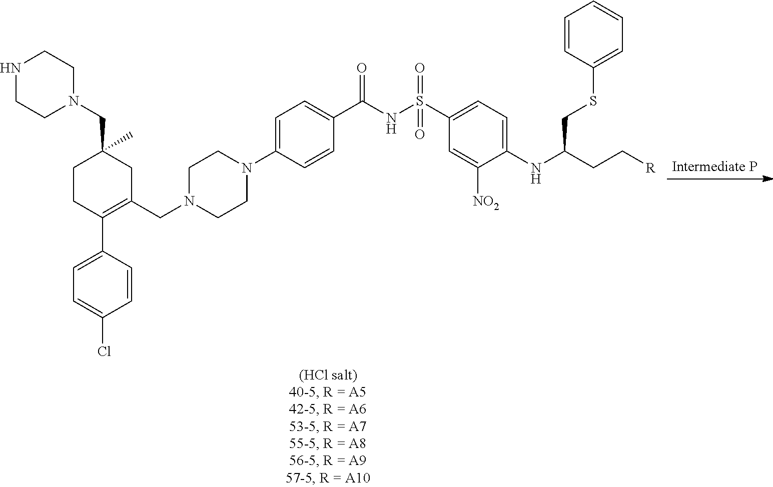

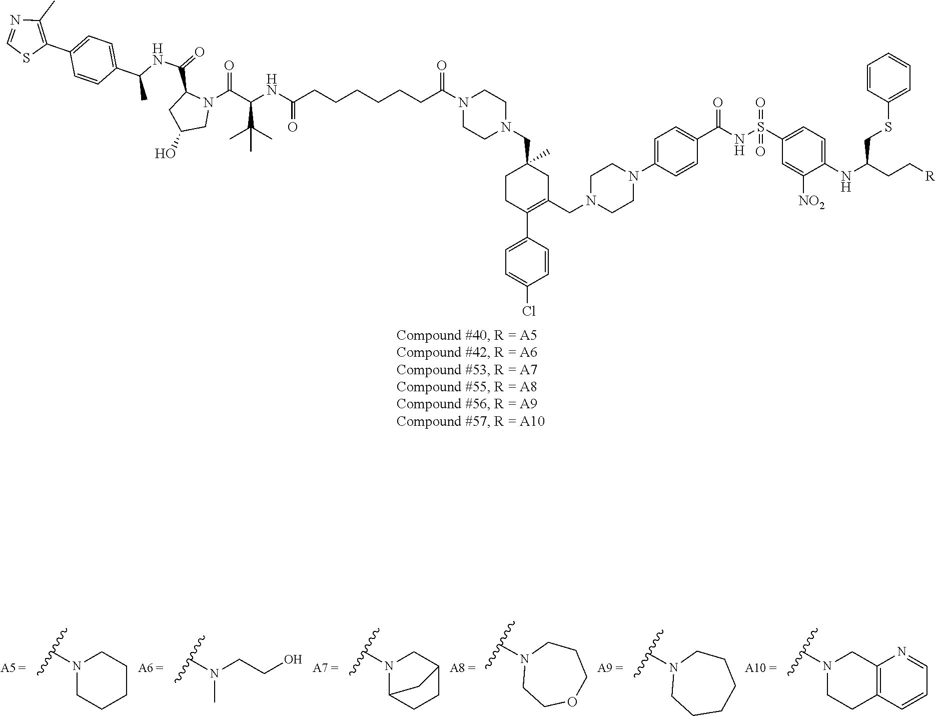

Compounds #40, #42, #53, and #55-57 were prepared by following General Procedure D. tert-butyl ((2R)-4-(2-azabicyclo[2.2.1]heptan-2-yl)-1-(phenylthio)butan-2-yl)carbamate (53-1) (30 mg, 79% yield). ¹H NMR (600 MHz, Methanol-d₄) δ 7.44 (d, J=7.7 Hz, 2H), 7.33 (t, J=7.6 Hz, 2H), 7.24 (t, J=7.4 Hz, 1H), 4.00 (d, J=17.4 Hz, 1H), 3.75-3.60 (m, 1H), 3.24-2.98 (m, 6H), 2.72 (d, J=6.1 Hz, 1H), 2.17-2.07 (m, 1H), 1.97 (d, J=11.7 Hz, 1H), 1.91-1.72 (m, 5H), 1.55 (dd, J=15.2, 8.6 Hz, 1H), 1.46 (s, 9H) ppm. LC/MS (ESI) m/z 376.3; [M+H]⁺ calcd for C₂₁H₃₂N₂O₂S⁺: 376.22.

tert-butyl (R)-(4-(1,4-oxazepan-4-yl)-1-(phenylthio)butan-2-yl)carbamate (55-1) (22 mg, 57% yield). ¹H NMR (600 MHz, Methanol-d₄) δ 7.42 (d, J=7.7 Hz, 2H), 7.31 (t, J=7.7 Hz, 2H), 7.20 (t, J=7.4 Hz, 1H), 3.82-3.66 (m, 5H), 3.12 (dd, J=13.6, 6.4 Hz, 1H), 3.02 (dd, J=13.5, 6.7 Hz, 1H), 2.80-2.69 (m, 4H), 2.65-2.55 (m, 2H), 1.99-1.83 (m, 3H), 1.71-1.57 (m, 1H), 1.45 (s, 9H) ppm. LC/MS (ESI) m/z 381.0; [M+H]⁺ calcd for C₂₀H₃₃N2O₃S⁺: 381.22.

tert-butyl (R)-(4-(azepan-1-yl)-1-(phenylthio)butan-2-yl)carbamate (56-1) (21 mg, 55% yield). ¹H NMR (600 MHz, Methanol-d₄) δ 7.45 (d, 2H), 7.33 (t, J=7.8 Hz, 2H), 7.26-7.21 (m, 1H), 3.72-3.63 (m, 1H), 3.28-3.19 (m, 4H), 3.17-3.02 (m, 4H), 2.21-2.13 (m, 1H), 1.95-1.79 (m, 5H), 1.73 (p, J=3.0 Hz, 4H), 1.46 (s, 9H) ppm. LC/MS (ESI) m/z 379.4; [M+H]⁺ calcd for C₂₁H₃₅N₂O₂S⁺: 379.24.

tert-butyl (R)-(4-(5,8-dihydro-1,7-naphthyridin-7(6H)-yl)-1-(phenylthio)butan-2-yl)carbamate (57-1) (22 mg, 16% yield). ¹H NMR (600 MHz, Methanol-d₄) δ 8.31 (dd, J=5.0, 1.5 Hz, 1H), 7.61 (d, J=7.7 Hz, 1H), 7.42 (d, J=7.8 Hz, 2H), 7.30-7.20 (m, 3H), 7.17 (t, J=7.4 Hz, 1H), 3.86-3.75 (m, 1H), 3.72 (q, 2H), 3.17-3.02 (m, 2H), 2.92 (q, J=5.8 Hz, 2H), 2.79 (t, J=6.1 Hz, 2H), 2.70-2.59 (m, 2H), 2.09-2.00 (m, 1H), 1.80-1.68 (m, 1H), 1.44 (s, 9H) ppm. LC/MS (ESI) m/z 414.5; [M+H]⁺ calcd for C₂₃H₃₂N₃O₂S⁺: 414.22.

(2R)-4-(2-azabicyclo[2.2.1]heptan-2-yl)-1-(phenylthio)butan-2-amine hydrochloride (53-2) (30 mg, quantitative yield). LC/MS (ESI) m/z 277.2; [M+H]⁺ calcd for C₁₆H₂₅N₂S⁺: 277.17.

(R)-4-(1,4-oxazepan-4-yl)-1-(phenylthio)butan-2-amine hydrochloride (55-2) (24 mg, quantitative yield). LC/MS (ESI) m/z 281.0; [M+H]⁺ calcd for C₁₅H₂₅N₂OS⁺: 281.17.

(R)-4-(azepan-1-yl)-1-(phenylthio)butan-2-amine hydrochloride (56-2) (24 mg, quantitative yield). LC/MS (ESI) m/z 279.3; [M+H]⁺ calcd for C₁₆H₂₇N₂S⁺: 279.19. (R)-4-(5,8-dihydro-1,7-naphthyridin-7(6H)-yl)-1-(phenylthio)butan-2-amine hydrochloride (57-2) (31 mg, quantitative yield). LC/MS (ESI) m/z 314.3; [M+H]⁺ calcd for C₁₈H₂₄N₃S⁺: 314.17.

4-(((2R)-4-(2-azabicyclo[2.2.1]heptan-2-yl)-1-(phenylthio)butan-2-yl)amino)-3-nitrobenzenesulfonamide (53-3) (6.7 mg, 28% yield). ¹H NMR (600 MHz, Chloroform-d) δ 8.79 (s, 1H), 8.70 (dt, J=6.3, 1.9 Hz, 1H), 7.73 (d, J=9.0 Hz, 1H), 7.41-7.36 (m, 2H), 7.33-7.24 (m, 2H), 6.82 (dd, J=13.8, 9.2 Hz, 1H), 4.10 (s, 1H), 3.30-3.10 (m, 3H), 2.99-2.52 (m, 3H), 2.39 (s, 1H), 2.34-2.12 (m, 1H), 2.02 (s, 1H), 1.95-1.82 (m, 1H), 1.76 (d, J=9.7 Hz, 2H), 1.71-1.54 (m, 2H), 1.46 (dd, J=25.0, 12.0 Hz, 1H), 1.37-1.21 (m, 3H) ppm. LC/MS (ESI) m/z 477.0; [M+H]⁺ calcd for C₂₂H₂₉N₄O₄S₂⁺: 477.16.

(R)-4-((4-(1,4-oxazepan-4-yl)-1-(phenylthio)butan-2-yl)amino)-3-nitrobenzenesulfonamide (55-3) (9.7 mg, 70% yield in two steps). ¹H NMR (600 MHz, Chloroform-d) δ 8.76-8.65 (m, 2H), 7.74 (dd, J=9.1, 2.3 Hz, 1H), 7.41-7.36 (m, 2H), 7.34-7.24 (m, 3H), 6.78 (d, J=9.2 Hz, 1H), 4.90 (brs, 2H), 4.13-4.03 (m, 1H), 3.79 (t, J=6.0 Hz, 2H), 3.75-3.66 (m, 2H), 3.17 (d, J 14=6.1 Hz, 2H), 2.79-2.59 (m, 6H), 2.16-2.07 (m, 1H), 1.92-1.77 (m, 3H) ppm. LC/MS (ESI) m/z 481.3; [M+H]⁺ calcd for C₂₁H₂₉N₄O₅S₂⁺: 481.16.

(R)-4-((4-(azepan-1-yl)-1-(phenylthio)butan-2-yl)amino)-3-nitrobenzenesulfonamide (56-3) (10 mg, 75% yield in two steps). ¹H NMR (600 MHz, Chloroform-d) δ 8.70 (d, J=2.3 Hz, 1H), 8.64 (d, J=8.5 Hz, 1H), 7.74 (dd, J=9.2, 2.3 Hz, 1H), 7.40-7.35 (m, 2H), 7.32-7.23 (m, 3H), 6.84 (d, J=9.2 Hz, 1H), 4.92 (brs, 2H), 4.14 (d, J=7.1 Hz, 1H), 3.27-3.11 (m, 2H), 2.85-2.60 (m, 6H), 2.17-2.09 (m, 1H), 1.88 (s, 1H), 1.74-1.56 (m, 8H) ppm. LC/MS (ESI) m/z 479.3; [M+H]⁺ calcd for C₂₂H₃₁N4O₄S₂⁺: 479.18.

(R)-4-((4-(5,8-dihydro-1,7-naphthyridin-7(6H)-yl)-1-(phenylthio)butan-2-yl)amino)-3-nitrobenzenesulfonamide (57-3) (13 mg, 48% yield in two steps). ¹H NMR (600 MHz, Chloroform-d) δ 8.65 (dq, J=5.9, 2.3 Hz, 1H), 8.57 (d, J=9.0 Hz, 1H), 8.40-8.34 (m, 1H), 7.61 (dq, J=9.2, 2.6, 2.2 Hz, 1H), 7.46 (dd, J=7.7, 1.6 Hz, 1H), 7.39-7.33 (m, 2H), 7.26-7.20 (m, 3H), 7.13 (dd, J=7.6, 4.9 Hz, 1H), 6.78 (d, J=9.2 Hz, 1H), 5.18 (s, 2H), 4.16 (d, J=9.6 Hz, 1H), 3.73 (d, J=15.6 Hz, 1H), 3.57-3.48 (m, 1H), 3.20 (d, J=6.1 Hz, 2H), 2.94-2.80 (m, 2H), 2.80-2.72 (m, 1H), 2.72-2.57 (m, 3H), 2.31-2.19 (m, 1H), 1.95-1.84 (m, 1H) ppm. LC/MS (ESI) m/z 514.2; [M+H]⁺ calcd for C₂₄H₂₈N₅O₄S₂⁺: 514.16.

tert-butyl 4-(((R)-4′-chloro-4-methyl-6-((4-(4-(((3-nitro-4-(((R)-1-(phenylthio)-4-(piperidin-3-(1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (40-4) (31 mg, 90% yield). ¹H NMR (600 MHz, CDCl₃) δ 8.69 (dd, J=7.6, 2.2 Hz, 1H), 8.28 (dd, J=22.5, 8.6 Hz, 1H), 7.89-7.81 (m, 3H), 7.29-7.22 (m, 4H), 7.20-7.11 (m, 3H), 7.01-6.95 (m, 2H), 6.77-6.67 (m, 3H), 4.05 (d, J=9.4 Hz, 1H), 3.39 (t, J=5.0 Hz, 4H), 3.21 (q, J=4.9 Hz, 4H), 3.16-3.03 (m, 2H), 2.85 (d, J=43.0 Hz, 7H), 2.55-2.44 (m, 4H), 2.43-2.16 (m, 10H), 2.16-2.02 (m, 2H), 1.93 (d, J=17.4 Hz, 1H), 1.85 (s, 4H), 1.65-1.48 (m, 4H), 1.45 (s, 9H), 0.94 (s, 3H) ppm. LC/MS (ESI) m/z 1069.6; [M+H]⁺ calcd for C₅₆H₇₄ClN₈O₇S₂⁺: 1069.48.

tert-butyl 4-(((R)-4′-chloro-6-((4-(4-(((4-(((R)-4-((2-hydroxyethyl)(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (42-4) (10 mg, 29% yield). ¹H NMR (600 MHz, CDCl₃) δ 8.77-8.67 (m, 1H), 8.63 12-8.50 (m, 1H), 7.90 (d, J=9.5 Hz, 1H), 7.79 (d, J=8.6 Hz, 1H), 7.40-7.26 (m, 5H), 7.26-7.15 (m, 3H), 7.01-6.98 (m, 2H), 6.75 (d, J=8.9 Hz, 2H), 6.67 (d, J=9.3 Hz, 1H), 4.00 (s, 1H), 3.79-3.65 (m, 2H), 3.41 (s, 4H), 3.29-3.20 (m, 4H), 3.18-3.08 (m, 2H), 2.94-2.66 (m, 6H), 2.57-2.27 (m, 12H), 2.26-2.09 (m, 5H), 2.02-1.89 (m, 2H), 1.68-1.56 (m, 2H), 1.47 (s, 9H), 0.96 (d, J=2.9 Hz, 3H) ppm. LC/MS (ESI) m/z 1059.4; [M+H]⁺ calcd for C₅₄H₇₂ClN₈O₈S₂⁺: 1059.46.

tert-butyl 4-(((4R)-6-((4-(4-(((4-(((2R)-4-(2-azabicyclo[2.2.1]heptan-2-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (53-4) (10 mg, 64% yield). ¹H NMR (600 MHz, CDCl₃) δ 8.62 (dd, J=10.7, 2.3 Hz, 1H), 7.92 (d, J=8.4 Hz, 2H), 7.83 (dd, 1H), 7.32-7.23 (m, 4H), 7.22-7.10 (m, 3H), 7.02-6.96 (m, 2H), 6.84-6.73 (m, 3H), 4.20-4.06 (m, 1H), 3.97 (s, 1H), 3.40 (q, J=6.8 Hz, 4H), 3.34-2.85 (m, 8H), 2.80 (s, 2H), 2.62 (s, 1H), 2.49 (s, 4H), 2.43-2.06 (m, 12H), 2.04-1.77 (m, 5H), 1.77-1.64 (m, 2H), 1.64-1.51 (m, 3H), 1.45 (s, 9H), 0.95 (s, 3H) ppm. LC/MS (ESI) m/z 1081.7; [M+H]⁺ calcd for C₅₇H₇₄ClN₈O₇S₂⁺: 1081.48.

tert-butyl 4-(((R)-6-((4-(4-(((4-(((R)-4-(1,4-oxazepan-4-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (55-4) (16.3 mg, 72% yield). ¹H NMR (600 MHz, CDCl₃) δ 8.74 (d, J=2.7 Hz, 1H), 7.95 (d, J=8.8 Hz, 1H), 7.75 (d, J=8.4 Hz, 2H), 7.35-7.25 (m, 4H), 7.25-7.17 (m, 3H), 7.01-6.97 (m, 2H), 6.77 (d, J 34=8.6 Hz, 2H), 6.71 (d, J=9.3 Hz, 1H), 4.03 (s, 1H), 3.82-3.71 (m, 4H), 3.41 (t, J=5.0 Hz, 4H), 3.26 (t, J=5.2 Hz, 4H), 3.15 (d, J=5.7 Hz, 2H), 2.92-2.68 (m, 8H), 2.51 (s, 4H), 2.43-2.09 (m, 12H), 2.01-1.86 (m, 3H), 1.64-1.55 (m, 1H), 1.46 (s, 9H), 0.96 (s, 3H) ppm. LC/MS (ESI) m/z 1085.6; [M+H]⁺ calcd for C₅₆H₇₄ClN₈O₈S₂⁺: 1085.48.

tert-butyl 4-(((R)-6-((4-(4-(((4-(((R)-4-(azepan-1-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (56-4) (14 mg, 62% yield). ¹H NMR (600 MHz, CDCl₃) δ 8.66 (d, J=2.2 Hz, 1H), 7.88 (d, J=8.9 Hz, 2H), 7.81 (dd, J=9.1, 2.2 Hz, 1H), 7.31-7.24 (m, 4H), 7.21-7.12 (m, 3H), 7.01-6.97 (m, 2H), 6.79-6.71 (m, 3H), 4.08 (t, J=6.0 Hz, 1H), 3.40 (t, J=5.0 Hz, 4H), 3.26-2.98 (m, 12H), 2.81 (s, 2H), 2.56-2.44 (m, 4H), 2.42-2.08 (m, 12H), 1.93 (d, J=17.3 Hz, 1H), 1.89-1.71 (m, 6H), 1.68-1.55 (m, 4H), 1.45 (s, 9H), 0.95 (s, 3H) ppm. LC/MS (ESI) m/z 1083.6; [M+H]⁺ calcd for C₅₇H₇₆ClN₈O₇S₂⁺: 1083.50.

tert-butyl 4-(((R)-4′-chloro-6-((4-(4-(((4-(((R)-4-(5,8-dihydro-1,7-naphthyridin-7(6H)-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (57-4) (12 mg, 42% yield). ¹H NMR (600 MHz, CDCl₃) δ 8.82-8.76 (m, 1H), 8.66 (d, J=8.4 Hz, 1H), 8.38 (dd, J=4.8, 1.7 Hz, 1H), 7.95 (dd, J=9.1, 2.5 Hz, 1H), 7.71-7.63 (m, 2H), 7.45 (dd, J=7.7, 1.6 Hz, 1H), 7.35-7.30 (m, 2H), 7.31-7.26 (m, 2H), 7.18 (dd, J=4.9, 2.1 Hz, 3H), 7.12 (dd, J=7.7, 4.8 Hz, 1H), 7.01-6.97 (m, 2H), 6.81-6.71 (m, 3H), 4.13-4.05 (m, 1H), 3.84 (d, J=15.7 Hz, 1H), 3.62 (dd, J=15.6, 3.2 Hz, 1H), 3.41 (t, J=5.0 Hz, 4H), 3.28 (t, J=5.1 Hz, 4H), 3.18 (d, J=5.8 Hz, 2H), 2.97-2.79 (m, 4H), 2.77-2.59 (m, 4H), 2.56-2.46 (m, 4H), 2.43-2.18 (m, 10H), 2.15 (d, J=17.4 Hz, 1H), 1.98-1.88 (m, 2H), 1.66-1.56 (m, 1H), 1.47 (s, 9H), 0.95 (s, 3H) ppm. LC/MS (ESI) m/z 1118.3; [M+H]⁺ calcd for C₅₉H₇₃ClN₉O₇S₂⁺: 1118.48.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((3-nitro-4-(((R)-1-(phenylthio)-4-(piperidin-1-yl)butan-2-yl)amino)phenyl)sulfonyl)benzamide hydrochloride (40-5) (28 mg, quantitative yield). LC/MS (ESI) m/z 969.5; [M+H]⁺ calcd for C₅₁H₆₆ClN₈O₅S₂⁺: 969.43.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-((2-hydroxyethyl)(methyl)amino)-1-(phenylthio)butan-2-yl)amino)-3-nitrophenyl)sulfonyl)benzamide amine hydrochloride (42-5) (13 mg, quantitative yield). LC/MS (ESI) m/z 959.3; [M+H]⁺ calcd for C₄₉H64ClN₈O₆S₂⁺: 959.41.

N-((4-(((2R)-4-(2-azabicyclo[2.2.1]heptan-2-yl)-1-(phenylthio)butan-2-yl)amino)-3-nitrophenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (53-5) (10 mg, quantitative yield). LC/MS (ESI) m/z 981.3; [M+H]⁺ calcd for C₅₂H₆₆ClN₈O₅S₂⁺: 981.43.

N-((4-(((R)-4-(1,4-oxazepan-4-yl)-1-(phenylthio)butan-2-yl)amino)-3-nitrophenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (55-5) (18 mg, quantitative yield). LC/MS (ESI) m/z 985.6; [M+H]⁺ calcd for C₅₁H₆₆ClN₈O₆S₂⁺: 985.42.

N-((4-(((R)-4-(azepan-1-yl)-1-(phenylthio)butan-2-yl)amino)-3-nitrophenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (56-5) (15 mg, quantitative yield). LC/MS (ESI) m/z 983.2; [M+H]⁺ calcd for C₅₂H68ClN₈O₅S₂⁺: 983.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(5,8-dihydro-1,7-naphthyridin-7(6H)-yl)-1-(phenylthio)butan-2-yl)amino)-3-nitrophenyl)sulfonyl)benzamide hydrochloride (57-5) (14 mg, quantitative yield). LC/MS (ESI) m/z 1018.6; [M+H]⁺ calcd for C₅₄H₆₅ClN₉O₅S₂⁺: 1018.42.

(2S,4R)-1-((S)-2-(8-(4-(((R)-4′-chloro-4-methyl-6-((4-(4-(((3-nitro-4-(((R)-1-(phenylthio)-4-(piperidin-1-yl)butan-2-yl)amino)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #40) (21.3 mg, 59% yield in two steps). ¹H NMR (600 MHz, CDCl₃) δ 8.69 (s, 1H), 8.33 (s, 1H), 7.92-7.78 (m, 3H), 7.53 (d, J=7.9 Hz, 1H), 7.40 (q, J=8.1 Hz, 4H), 7.34-7.25 (m, 3H), 7.23-7.13 (m, 3H), 7.01 (d, J=8.0 Hz, 2H), 6.80-6.69 (m, 3H), 6.32 (s, 1H), 5.10 (t, J=7.2 Hz, 1H), 4.76 (t, J=7.9 Hz, 1H), 4.61 (d, J=8.7 Hz, 1H), 4.52 (s, 1H), 4.12 (d, J=11.4 Hz, 1H), 4.04 (s, 1H), 3.64-3.54 (m, 2H), 3.43 (s, 2H), 3.27-3.06 (m, 7H), 2.98-2.67 (m, 7H), 2.65-2.00 (m, 26H), 1.92 (d, J=17.2 Hz, 1H), 1.79 (s, 4H), 1.66-1.39 (m, 10H), 1.28 (s, 6H), 1.06 (s, 9H), 0.96 (s, 3H) ppm. LC/MS (ESI) m/z 1551.6; [M+H]⁺ calcd for Cs₂H₁₀₈ClN₁₂O₁₀S₃⁺: 1551.72.

(2S,4R)-1-((S)-2-(8-(4-(((R)-4′-chloro-6-((4-(4-(((4-(((R)-4-((2-hydroxyethyl)(methyl)amino)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #42) (5.9 mg, 41% yield in two steps). ¹H NMR (600 MHz, CDCl₃) δ 8.74-8.66 (m, 2H), 8.57 (s, 1H), 7.92 (d, J=8.9 Hz, 1H), 7.79 (d, J=8.5 Hz, 2H), 7.52 (d, J=7.9 Hz, 1H), 7.40 (q, J=8.4 Hz, 4H), 7.34-7.26 (m, 3H), 7.25-7.14 (m, 3H), 7.01 (d, J=8.1 Hz, 2H), 6.72 (dd, J=25.1, 8.9 Hz, 3H), 6.37 (s, 1H), 5.11 (p, J=7.1 Hz, 1H), 4.76 (t, J=8.0 Hz, 1H), 4.62 (d, J=8.9 Hz, 1H), 4.52 (s, 1H), 4.11 (d, J=11.4 Hz, 1H), 4.02 (s, 1H), 3.78-3.66 (m, 2H), 3.65-3.53 (m, 2H), 3.43 (s, 2H), 3.22 (s, 4H), 3.14 (s, 2H), 2.92-2.70 (m, 6H), 2.65-2.06 (m, 28H), 1.99 (s, 1H), 1.91 (d, J=17.2 Hz, 1H), 1.69-1.52 (m, 6H), 1.52-1.41 (m, 4H), 1.38-1.22 (m, 5H), 1.06 (s, 9H), 0.96 (s, 3H) ppm. LC/MS (ESI) m/z 1541.5; [M+H]⁺ calcd for C₈₀H₁₀₆ClN₁₂O₁₁S₃⁺: 1541.69.

(2S,4R)-1-((2S)-2-(8-(4-(((4R)-6-((4-(4-(((4-(((2R)-4-(2-azabicyclo[2.2.1]heptan-2-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #53) (4.3 mg, 30% yield in two steps). ¹H NMR (600 MHz, CDCl₃) δ 8.69 (s, 1H), 8.62 (d, J=10.1 Hz, 1H), 8.23 (d, J=33.1 Hz, 1H), 7.96-7.75 (m, 3H), 7.52 (d, J=7.9 Hz, 1H), 7.40 (q, J=8.1 Hz, 4H), 7.34-7.24 (m, 3H), 7.23-7.12 (m, 3H), 7.01 (d, J=8.0 Hz, 2H), 6.83-6.70 (m, 3H), 6.36 (s, 1H), 5.10 (p, J=7.1 Hz, 1H), 4.76 (t, J=8.0 Hz, 1H), 4.61 (d, J=8.8 Hz, 1H), 4.52 (s, 1H), 4.19-4.04 (m, 1H), 3.95 (s, 1H), 3.64-3.54 (m, 2H), 3.44 (s, 2H), 3.32-3.00 (m, 7H), 2.96-2.78 (m, 3H), 2.70-2.12 (m, 28H), 1.93 (d, J=17.2 Hz, 1H), 1.75-1.52 (m, 10H), 1.52-1.41 (m, 4H), 1.41-1.20 (m, 7H), 1.06 (s, 9H), 0.96 (s, 3H) ppm. LC/MS (ESI) m/z 1563.9; [M+H]⁺ calcd for C₈₃H₁₀₈ClN₁₂O₁₀S₃⁺: 1563.72.

(2S,4R)-1-((S)-2-(8-(4-(((R)-6-((4-(4-(((4-(((R)-4-(1,4-oxazepan-4-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #55) (9.8 mg, 42% yield in two steps). ¹H NMR (600 MHz, CDCl₃) δ 8.77 (q, J=2.4 Hz, 1H), 8.70 (s, 1H), 8.68-8.58 (m, 1H), 7.98 (dd, J=9.7, 3.5 Hz, 1H), 7.78-7.71 (m, 2H), 7.49 (d, J=7.8 Hz, 1H), 7.40 (q, 4H), 7.34 (d, 2H), 7.30 (d, 1H), 7.27-7.18 (m, 3H), 7.00 (d, J=8.2 Hz, 2H), 6.77 (d, J=8.6 Hz, 2H), 6.73 (dd, J=9.5, 3.1 Hz, 1H), 6.33 (s, 1H), 5.11 (p, J=7.2 Hz, 1H), 4.76 (t, J=8.0 Hz, 1H), 4.64 (d, J=8.8 Hz, 1H), 4.52 (d, J=4.1 Hz, 1H), 4.14 (d, J=11.3 Hz, 1H), 4.04 (s, 1H), 3.79 (t, J=6.1 Hz, 2H), 3.77-3.69 (m, 2H), 3.65-3.53 (m, 3H), 3.43 (t, J=5.4 Hz, 2H), 3.25 (t, J=5.4 Hz, 4H), 3.16 (d, J=5.7 Hz, 2H), 2.91-2.64 (m, 9H), 2.64-2.46 (m, 7H), 2.45-2.33 (m, 4H), 2.33-2.17 (m, 8H), 2.11 (dd, J=13.7, 8.2 Hz, 2H), 2.02-1.81 (m, 4H), 1.69-1.53 (m, 6H), 1.53-1.41 (m, 4H), 1.39-1.24 (m, 7H), 1.06 (s, 9H), 0.96 (s, 3H) ppm. LC/MS (ESI) m/z 1567.5; [M+H]⁺ calcd for C₈₂H₁₀₈ClN₁₂O₁₁S₃⁺: 1567.71.

(2S,4R)-1-((S)-2-(8-(4-(((R)-6-((4-(4-(((4-(((R)-4-(azepan-1-yl)-1-(phenylthio)butan-2-yl)amino)-3-nitrophenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-1-(3,3-dimethylbutanoyl)-4-hydroxy-N—((S)-1-(4-(4-methylthiazol-5-yl)phenyl)ethyl)pyrrolidine-2-carboxamide (compound #56) (9.3 mg, 46% yield in two steps). ¹H NMR (600 MHz, CDCl₃) δ 8.69 (s, 1H), 8.66 (s, 1H), 8.31 (s, 1H), 7.87 (d, J=8.2 Hz, 2H), 7.81 (d, J=9.0 Hz, 1H), 7.52 (d, J=7.9 Hz, 1H), 7.40 (q, J=8.3 Hz, 4H), 7.29 (p, J=8.3 Hz, 3H), 7.23-7.13 (m, 3H), 7.01 (d, J=8.3 Hz, 2H), 6.79-6.71 (m, 3H), 6.34 (s, 1H), 5.11 (p, J=7.1 Hz, 1H), 4.76 (t, J=8.0 Hz, 1H), 4.62 (d, J=8.8 Hz, 1H), 4.52 (s, 1H), 4.15-4.05 (m, 2H), 3.64-3.54 (m, 2H), 3.44 (s, 2H), 3.22 (s, 4H), 3.18-2.95 (m, 8H), 2.85 (s, 2H), 2.54 (s, 8H), 2.43-2.07 (m, 16H), 1.93 (d, J=17.2 Hz, 1H), 1.83 (s, 4H), 1.73-1.54 (m, 10H), 1.52-1.42 (m, 4H), 1.38-1.23 (m, 7H), 1.06 (s, 9H), 0.96 (s, 3H) ppm. LC/MS (ESI) m/z 1565.4; [M+H]+ calcd for C₈₃H₁₁₀ClN₁₂O₁₀S₃⁺: 1565.73.

(2S,4R)-1-((S)-2-(8-(4-(((R)-4′-chloro-6-((4-(4-(((4-(((R)-4-(5,8-dihydro-1,7-naphthyridin-7(6H)-yl)-1-(phenylthio)butan-2-yl)amino)-3-nitrophenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #57) (14.6 mg, 85% yield in two steps). ¹H NMR (600 MHz, CDCl₃) δ 8.78-8.74 (m, 1H), 8.69 (s, 1H), 8.65 (d, J=8.6 Hz, 1H), 8.40-8.36 (m, 1H), 7.96 (dd, J=9.3, 2.3 Hz, 1H), 7.72 (d, J=8.5 Hz, 2H), 7.50 (d, J=7.9 Hz, 1H), 7.45 (dd, J=7.8, 1.6 Hz, 1H), 7.40 (q, J=8.2 Hz, 4H), 7.35-7.26 (m, 4H), 7.20-7.16 (m, 3H), 7.12 (dd, J=7.7, 4.8 Hz, 1H), 7.02-6.97 (m, 2H), 6.76 (t, J=8.7 Hz, 3H), 6.33 (s, 1H), 5.11 (p, J=7.1 Hz, 1H), 4.76 (t, J=8.0 Hz, 1H), 4.64 (d, J=8.8 Hz, 1H), 4.51 (d, J=4.9 Hz, 1H), 4.18-4.06 (m, 2H), 3.82 (d, J=15.7 Hz, 1H), 3.65-3.52 (m, 5H), 3.43 (t, J=5.2 Hz, 2H), 3.28-3.20 (m, 4H), 3.18 (d, J=5.9 Hz, 2H), 2.96-2.79 (m, 4H), 2.76-2.45 (m, 11H), 2.44-2.15 (m, 14H), 2.14-2.07 (m, 1H), 1.97-1.87 (m, 2H), 1.67-1.54 (m, 6H), 1.53-1.41 (m, 4H), 1.38-1.22 (m, 5H), 1.06 (s, 9H), 0.96 (s, 3H) ppm. LC/MS (ESI) m/z 1600.7; [M+H]⁺ calcd for C₈₅H₁₀₇ClN₁₃O₁₀S₃⁺: 1600.71.

### Example 28: Preparation of Compounds #58-60, #62, #67, and #71

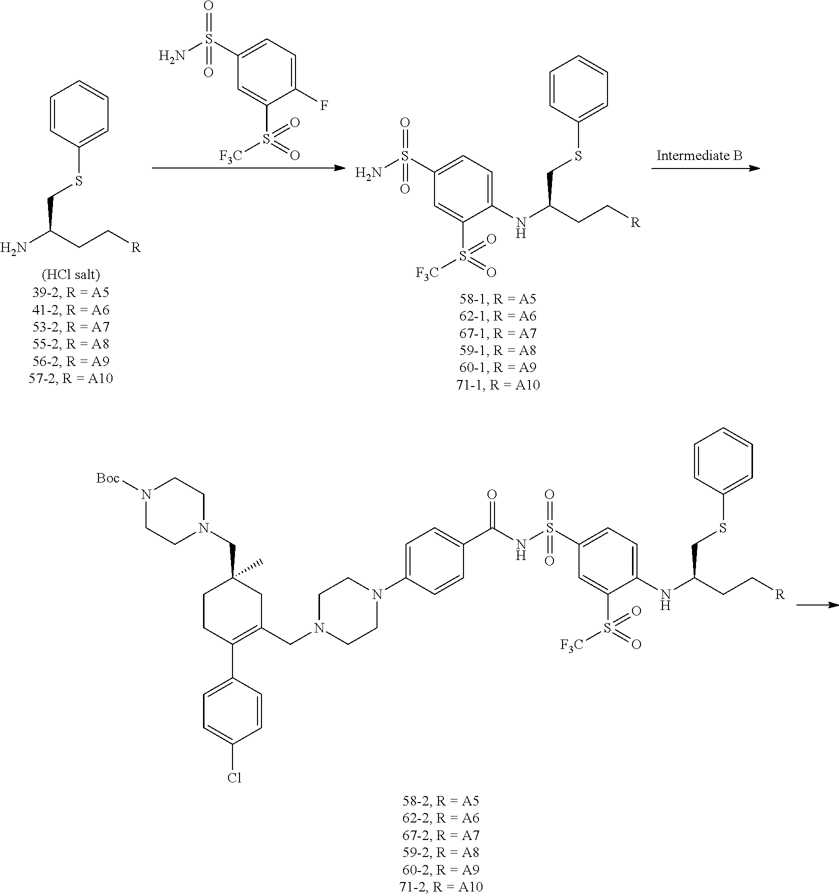

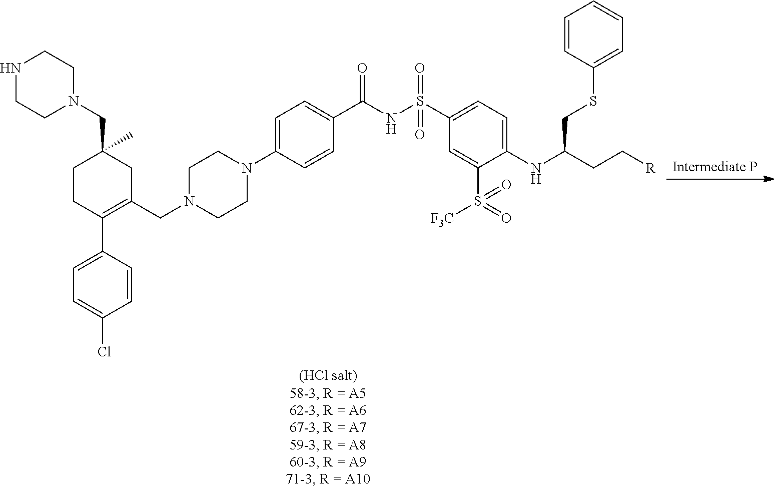

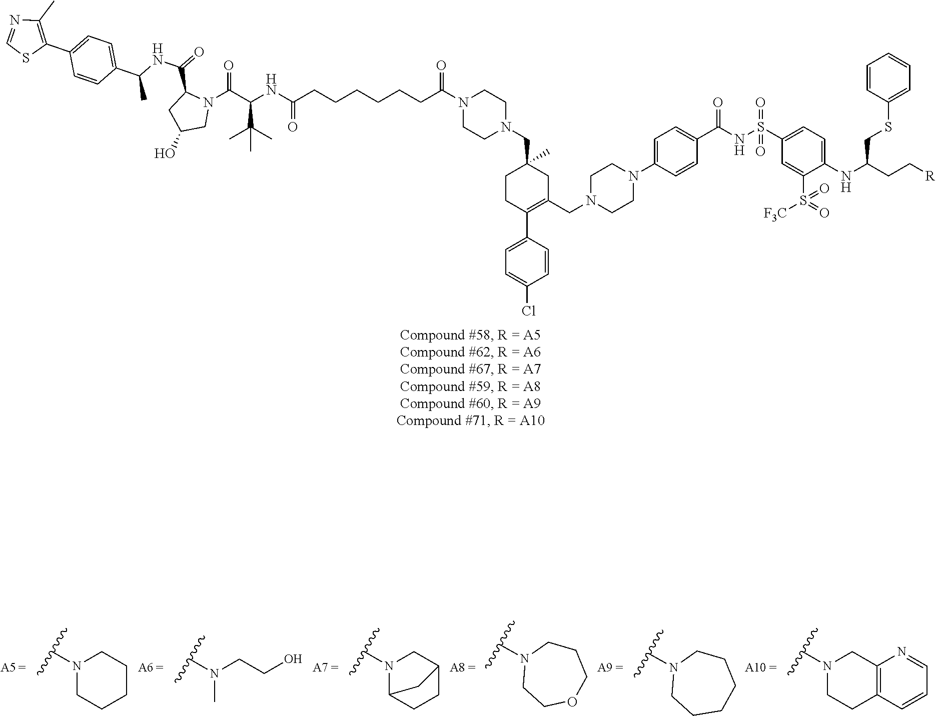

Compounds #58-60, #62, #67, and #71 were prepared by following General Procedure J. (R)-4-((1-(phenylthio)-4-(piperidin-1-yl)butan-2-yl)amino)-3-((trifluoromethyl)sulfonyl)benzenesulfonamide (58-1) (45 mg, 83% yield). ¹H NMR (600 MHz, Chloroform-d) δ 8.25 (d, J=2.3 Hz, 1H), 7.82 (dd, J=9.2, 2.3 Hz, 1H), 7.44-7.38 (m, 2H), 7.37-7.31 (z, 2H), 7.32-7.26 (, 1H), 7.05 (d, J=8.7 Hz, 1H), 6.72 (d, J=9.3 Hz, 1H), 3.94 (dp, J=9.7, 5.8, 5.3 Hz, 1H), 3.15-3.04 (m, 2H), 2.47-2.21 (m, 4H), 2.14-2.04 (in, 1H), 1.70 (ddt, J=14.1, 8.0, 5.8 Hz, 1H), 1.55 (dq, J=10.9, 5.4 Hz, 4H), 1.48-1.40 (, 2H) ppm. LC/MS (ESI) m/z 552.3; [M+H]⁺ calcd for C₂₂H₉F₃N₃O₄S₃⁺: 552.13.

(R)-4-((4-((2-hydroxyethyl)(methyl)amino)-1-(phenylthio)butan-2-yl)amino)-3-((trifluoromethyl)sulfonyl)benzenesulfonamide (62-1) (13 mg, 71% yield). ¹H NMR (600 MHz, Methanol-d₄) δ 8.14 (d, J=2.3 Hz, 1H), 7.89 (dd, J=9.2, 2.3 Hz, 1H), 7.43-7.37 (in, 2H), 7.30 (t, J=7.6 Hz, 2H), 7.27-7.21 (m, 1H), 6.91 (d, J=9.3 Hz, 1H), 4.07 (dq, J=8.2, 5.4 Hz, 1H), 3.63 (td, J=6.0, 1.6 Hz, 2H), 3.30 (dd, 1H), 3.19 (dd, J=14.2, 5.7 Hz, 1H), 2.63-2.47 (m, 4H), 2.30 (s, 3H), 2.07 (dtd, J=14.1, 7.8, 4.5 Hz, 1H), 1.83 (dtd, J=13.4, 8.1, 4.9 Hz, 1H) ppm. LC/MS (ESI) m/z 542.0; [M+H]⁺ calcd for C₂₀H₂₇F3N₃O₅S₃⁺: 542.11.

4-(((2R)-4-(2-azabicyclo[2.2.1]heptan-2-yl)-1-(phenylthio)butan-2-yl)amino)-3-((trifluoromethyl)sulfonyl)benzenesulfonamide (67-1) (12 mg, 47% yield). ¹H NMR (600 MHz, Methanol-d₄) δ 8.31 (dd, J=4.8, 1.5 Hz, 1H), 8.13 (d, J=2.3 Hz, 1H), 7.77 (dd, J=9.2, 2.3 Hz, 1H), 7.61 (dd, J=7.8, 1.6 Hz, 1H), 7.41-7.36 (m, 2H), 7.27-7.17 (m, 4H), 6.81 (d, J=9.3 Hz, 1H), 4.15-4.07 (m, 1H), 3.75 (d, J=15.7 Hz, 1H), 3.57 (d, J=15.8 Hz, 1H), 3.32 (dd, 1H), 3.20 (dd, J=14.2, 6.0 Hz, 1H), 2.95-2.82 (m, 2H), 2.79 (ddd, J=11.8, 6.9, 5.0 Hz, 1H), 2.74-2.65 (m, 2H), 2.63 (ddd, J=12.5, 6.9, 5.0 Hz, 1H), 2.26-2.18 (m, 1H), 1.94-1.84 (m, 1H) ppm. LC/MS (ESI) m/z 564.0; [M+H]⁺ calcd for C₂₃H₂₉F3N₃O₄S₃⁺: 564.13.

(R)-4-((4-(1,4-oxazepan-4-yl)-1-(phenylthio)butan-2-yl)amino)-3-((trifluoromethyl)sulfonyl)benzenesulfonamide (59-1) (13 mg, 79% yield). ¹H NMR (600 MHz, Chloroform-d) δ 8.26 (t, J=2.1 Hz, 1H), 7.86-7.79 (m, 1H), 7.42 (dd, J=8.2, 1.4 Hz, 2H), 7.35 (t, J=7.2, 6.0, 1.1 Hz, 2H), 7.33-7.26 (m, 1H), 7.19 (q, J=5.8, 4.6 Hz, 1H), 6.64 (d, J=9.2 Hz, 1H), 3.95 (tt, J=8.4, 5.0 Hz, 1H), 3.83-3.73 (m, 2H), 3.73-3.60 (m, 2H), 3.15-3.02 (m, 2H), 2.75-2.68 (m, 2H), 2.69-2.51 (m, 4H), 2.14-2.04 (m, 1H), 1.86 (p, J=5.8 Hz, 2H), 1.72 (ddt, J=13.7, 8.1, 5.4 Hz, 1H) ppm. LC/MS (ESI) m/z 568.2; [M+H]⁺ calcd for C₂₂H₂₉F3N₃O₅S₃⁺: 568.12.

(R)-4-((4-(azepan-1-yl)-1-(phenylthio)butan-2-yl)amino)-3-((trifluoromethyl)sulfonyl)benzenesulfonamide (60-1) (13 mg, 83% yield). ¹H NMR (600 24 MHz, Chloroform-d) δ 8.23 (dt, J=4.8, 2.5 Hz, 1H), 7.80 (ddt, J=10.2, 7.2, 2.8 Hz, 1H), 7.44-7.38 (m, 2H), 7.36-7.24 (m, 3H), 7.16-7.05 (m, 1H), 6.77-6.66 (m, 1H), 4.04 (q, J=7.7, 7.1 26 Hz, 1H), 3.17-3.08 (m, 2H), 2.88-2.68 (m, 6H), 2.17-2.07 (m, 1H), 1.89-1.79 (m, 1H), 1.70 (s, 4H), 1.62 (s, 4H) ppm. LC/MS (ESI) m/z 566.2; [M+H]⁺ calcd for C₂₃H₃₁F₃N₃O₄S₃⁺: 566.14.

(R)-4-((4-(5,8-dihydro-1,7-naphthyridin-7(6H)-yl)-1-(phenylthio)butan-2-yl)amino)-3-((trifluoromethyl)sulfonyl)benzenesulfonamide (71-1) (12 mg, 75% yield). ¹H NMR (600 32 MHz, Methanol-d₄) δ 8.57 (dd, J=6.3, 2.3 Hz, 1H), 8.50 (ddd, J=8.8, 4.5, 2.3 Hz, 1H), 8.31 (d, J=2.4 Hz, 1H), 8.16 (t, J=1.7 Hz, 1H), 7.92 (dt, J=9.2, 2.6 Hz, 1H), 7.85 (dd, J=9.0, 2.4 Hz, 1H), 7.62 (d, J=9.1 Hz, 2H), 7.43-7.36 (m, 2H), 7.29 (t, J=7.7 Hz, 2H), 7.24 (dd, J=8.3, 6.3 Hz, 1H), 6.93 (dd, J=9.2, 6.3 Hz, 1H), 4.07 (ddt, J=10.9, 8.4, 4.0 Hz, 1H), 3.39-3.26 (m, 1H), 3.26-3.19 (m, 1H), 3.18-3.01 (m, 2H), 2.29-2.18 (m, 1H), 2.11-2.02 (m, 1H), 1.95-1.90 (m, 1H), 1.87-1.77 (m, 3H), 1.76-1.69 (m, 1H), 1.55-1.47 (m, 1H) ppm. LC/MS (ESI) m/z 601.0; [M+H]⁺ calcd for C₂₅H₂₈F₃N₄O₄S₃⁺: 601.12.

tert-butyl 4-(((R)-4′-chloro-4-methyl-6-((4-(4-(((4-(((R)-1-(phenylthio)-4-(piperidin-1-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (58-2) (25 mg, 67% yield). ¹H NMR (600 MHz, Chloroform-d) δ 8.31 (d, J=2.2 Hz, 1H), 7.96 (dd, J=9.1, 2.2 Hz, 1H), 7.87 (d, J=8.5 Hz, 2H), 7.32 (d, J=7.6 Hz, 2H), 7.30-7.20 (m, 4H), 7.18 (t, J=7.3 Hz, 1H), 6.99 (d, J=8.3 Hz, 2H), 6.80 (d, J=8.5 Hz, 1H), 6.76 (d, J=8.7 Hz, 2H), 6.66 (d, J=9.2 Hz, 1H), 3.95 (d, J=9.4 Hz, 1H), 3.40 (t, J=5.0 Hz, 4H), 3.21 (t, J=5.2 Hz, 4H), 3.08-2.98 (m, 2H), 2.94-2.70 (m, 6H), 2.56-2.44 (m, 4H), 2.44-2.07 (m, 13H), 2.01-1.90 (m, 2H), 1.84 (s, 4H), 1.65-1.49 (m, 2H), 1.48-1.42 (m, 10H), 0.94 (s, 3H) ppm. LC/MS (ESI) m/z 1156.7; [M+H]⁺ calcd for C₅₇H₇₄ClF₃N₇O₇S₃⁺: 1156.44.

tert-butyl 4-(((R)-4′-chloro-6-((4-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (62-2) (13 mg, 47% yield). ¹H NMR (600 MHz, Chloroform-d) δ 8.31 (t, J=2.7 Hz, 1H), 8.02 (dt, J=8.9, 3.0 Hz, 1H), 7.77 (d, 2H), 7.41-7.35 (m, 2H), 7.33-7.20 (m, 4H), 6.99 (dd, J=8.3, 1.7 Hz, 2H), 6.77 (d, 2H), 6.56 (d, J=9.4 Hz, 1H), 3.87 (q, J=6.3 Hz, 1H), 3.73-3.63 (m, 2H), 3.40 (t, J=5.0 Hz, 4H), 3.24 (t, J=5.3 Hz, 4H), 3.10 (dd, J=14.0, 4.8, 1.7 Hz, 1H), 3.03 (dd, J=13.7, 7.2 Hz, 1H), 2.88-2.80 (m, 2H), 2.77-2.61 (m, 5H), 2.50 (s, 4H), 2.45-2.34 (m, 7H), 2.32-2.10 (m, 8H), 1.92 (d, J=17.4 Hz, 2H), 1.89-1.77 (m, 1H), 1.64-1.54 (m, 1H), 1.46 (s, 9H), 0.94 (s, 3H) ppm. LC/MS (ESI) m/z 1146.6; [M+H]⁺ calcd for C₅₅H₇₂ClF₃N₇O₈S₃⁺: 1146.42.

tert-butyl 4-(((4R)-6-((4-(4-(((4-(((2R)-4-(2-azabicyclo[2.2.1]heptan-2-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (67-2) (13 mg, 52% yield). ¹H NMR (600 MHz, Chloroform-d) δ 8.31 (d, J=2.2 Hz, 1H), 7.94 (d, J=8.6 Hz, 3H), 7.39-7.14 (m, 7H), 7.04-6.98 (m, 3H), 6.85 (dd, J=14.4, 8.6 Hz, 1H), 6.77 (d, J=8.7 Hz, 3H), 4.01 (d, J=40.9 Hz, 2H), 3.41 (t, J=4.8 Hz, 4H), 3.32-3.14 (m, 6H), 3.13-3.02 (m, 2H), 2.97-2.79 (m, 4H), 2.68-2.57 (m, 1H), 2.51 (s, 4H), 2.45-2.27 (m, 6H), 2.27-2.19 (m, 4H), 2.19-2.08 (m, 2H), 1.95 (d, J=17.3 Hz, 2H), 1.78-1.52 (m, 4H), 1.47 (s, 9H), 0.96 (s, 3H) ppm. LC/MS (ESI) m/z 1168.7; [M+H]⁺ calcd for C₅₈H₇₄ClF₃N7O₇S₃⁺: 1168.44.

tert-butyl 4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (59-2) (16 mg, 57% yield). ¹H NMR (600 MHz, Chloroform-d) δ 8.32 (d, J=2.3 Hz, 1H), 8.05 (dd, J=9.2, 2.4 Hz, 1H), 7.75 (d, J=8.5 Hz, 2H), 7.37 (d, 2H), 7.33-7.20 (m, 5H), 7.01-6.95 (m, 2H), 6.78 (d, 2H), 6.58 (dd, J=9.5, 2.7 Hz, 1H), 3.89 (q, J=7.0, 6.2 Hz, 1H), 3.80-3.69 (m, 4H), 3.40 (t, J=4.9 Hz, 4H), 3.26 (t, J=4.9 Hz, 4H), 3.14-3.00 (m, 2H), 2.88-2.59 (m, 8H), 2.50 (s, 5H), 2.38 (q, J=5.5 Hz, 4H), 2.33-2.07 (m, 6H), 1.97-1.86 (m, 4H), 1.76 (dt, J=13.7, 7.2 Hz, 1H), 1.60 (dt, J=13.4, 7.8 Hz, 1H), 1.45 (s, 9H), 0.94 (s, 3H) ppm. LC/MS (ESI) m/z 1172.5; [M+H]⁺ calcd for C₅₇H₇₄ClF₃N₇O₈S₃⁺: 1172.44.

tert-butyl 4-(((R)-6-((4-(4-(((4-(((R)-4-(azepan-1-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (60-2) (13 mg, 46% yield). ¹H NMR (600 MHz, Chloroform-d) δ 8.28 (d, J=2.2 Hz, 1H), 7.89 (dd, J=17.8, 8.9 Hz, 3H), 7.35 (d, 2H), 7.31-7.22 (m, 4H), 7.22-7.17 (m, 1H), 6.99 (dd, J=8.5, 2.4 Hz, 2H), 6.76 (d, 2H), 6.71 (d, J=9.2 Hz, 1H), 4.02-3.93 (m, 1H), 3.39 (t, J=5.0 Hz, 4H), 3.21 (t, J=5.0 Hz, 4H), 3.14-2.95 (m, 6H), 2.90 (d, J=27.1 Hz, 1H), 2.82 (s, 2H), 2.49 (s, 5H), 2.41-2.25 (m, 5H), 2.24-2.07 (m, 5H), 2.06-1.96 (m, 1H), 1.93 (d, J=17.3 Hz, 1H), 1.81 (s, 4H), 1.68-1.55 (m, 5H), 1.50-1.41 (m, 10H), 0.95 (d, J=5.5 Hz, 3H) ppm. LC/MS (ESI) m/z 1170.4; [M+H]⁺ calcd for C₅₈H₇₆ClF₃N₇O₇S₃⁺: 1170.46.

tert-butyl 4-(((R)-4′-chloro-6-((4-(4-(((4-(((R)-4-(5,8-dihydro-1,7-naphthyridin-7(6H)-yl)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (71-2) (14 mg, 58% yield). ¹H NMR (600 MHz, Chloroform-d) δ 8.39 (d, J=2.0 Hz, 1H), 8.36 (q, J=2.4, 1.9 Hz, 1H), 8.06 (dt, J=9.3, 2.5 Hz, 1H), 7.69-7.62 (m, 2H), 7.45 (d, 1H), 7.40-7.36 (m, 2H), 7.33-7.27 (m, 2H), 7.26-7.19 (m, 4H), 7.13 (dd, J=7.7, 4.8 Hz, 1H), 7.01-6.98 (m, 2H), 6.79 (dt, J=9.4, 2.5 Hz, 2H), 6.58 (dd, J=9.5, 3.1 Hz, 1H), 3.98 (s, 1H), 3.84 (d, J=15.7 Hz, 1H), 3.59 (d, J=15.7 Hz, 1H), 3.42 (s, 4H), 3.29 (t, J=5.1 Hz, 4H), 3.16-3.04 (m, 2H), 2.94-2.78 (m, 4H), 2.72 (t, J=6.0 Hz, 2H), 2.68-2.56 (m, 2H), 2.51 (s, 4H), 2.39 (s, 4H), 2.34-2.19 (m, 5H), 2.15 (d, J=17.4 Hz, 1H), 1.94 (d, J=17.3 Hz, 1H), 1.87-1.77 (m, 1H), 1.67-1.55 (m, 1H), 1.47 (s, 9H), 0.95 (s, 3H) ppm. LC/MS (ESI) m/z 1205.3; [M+H]⁺ calcd for C₆₀H₇₃ClF₃N₈O₇S₃⁺: 1205.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-1-(phenylthio)-4-(piperidin-1-yl)butan-2-yl)amino)-3-((trifluoromethyl)sulfonyl)phenyl)sulfonyl)benzamide hydrochloride (58-3) (30 mg, quantitative yield). LC/MS (ESI) m/z 1055.2; [M+H]⁺ calcd for C₅₂H₆₆ClF₃N₇O₅S₃⁺: 1055.38.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide hydrochloride (62-3) (15 mg, quantitative yield). LC/MS (ESI) m/z 1046.6; [M+H]⁺ calcd for C₅₀H₆₄ClF₃N₇O₆S₃⁺: 1046.37.

N-((4-(((2R)-4-(2-azabicyclo[2.2.1]heptan-2-yl)-1-(phenylthio)butan-2-yl)amino)-3-((trifluoromethyl)sulfonyl)phenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (67-3) (15 mg, quantitative yield). LC/MS (ESI) m/z 1068.3; [M+H]⁺ calcd for C₅₃H₆₆ClF₃N₇O₅S₃⁺: 1068.39.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (59-3) (20 mg, quantitative yield). LC/MS (ESI) m/z 1072.2; [M+H]⁺ calcd for 28 C₅₂H₆₆ClF₃N₇O₆S₃⁺: 1072.39.

N-((4-(((R)-4-(azepan-1-yl)-1-(phenylthio)butan-2-yl)amino)-3-((trifluoromethyl)sulfonyl)phenyl)sulfonyl)-4-(4-(((R)-4′-chloro-4-methyl-4-(piperazin-1-ylmethyl)-3,4,5,6-tetrahydro-[1,1′-biphenyl]-2-yl)methyl)piperazin-1-yl)benzamide hydrochloride (60-3) (15 mg, quantitative yield). LC/MS (ESI) m/z 1070.4; [M+H]⁺ calcd for C₅₃H₆₈ClF₃N₇O₅S₃⁺: 1070.41.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(5,8-dihydro-1,7-naphthyridin-7(6H)-yl)-1-(phenylthio)butan-2-yl)amino)-3-((trifluoromethyl)sulfonyl)phenyl)sulfonyl)benzamide hydrochloride (71-3) (15 mg, quantitative yield). LC/MS (ESI) m/z 1105.5; [M+H]⁺ calcd for C₅₅H₆₅ClF₃N₈O₅S₃⁺: 1105.39.

(2S,4R)-1-((S)-2-(8-(4-(((R)-4′-chloro-4-methyl-6-((4-(4-(((4-(((R)-1-(phenylthio)-4-(piperidin-1-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #58) (12.2 mg, 34% yield for two steps). ¹H NMR (600 MHz, Chloroform-d) δ 8.69 (s, 1H), 8.31 (s, 1H), 7.99 (s, 1H), 7.89-7.78 (m, 2H), 7.51 (d, J=7.5 Hz, 1H), 7.43-7.32 (m, 6H), 7.32-7.16 (m, 3H), 7.04-6.98 (m, 2H), 6.94-6.83 (m, 1H), 6.76 (d, 2H), 6.67 (s, 1H), 6.30 (s, 1H), 5.11 (t, J=7.2 Hz, 1H), 4.76 (t, J=7.8 Hz, 1H), 4.61 (d, J=8.7 Hz, 1H), 4.52 (s, 1H), 4.17-4.07 (m, 1H), 3.94 (s, 1H), 3.64-3.53 (m, 2H), 3.43 (s, 2H), 3.23 (s, 4H), 3.08 (d, J=10.3 Hz, 2H), 2.96-2.67 (m, 7H), 2.61-2.37 (m, 10H), 2.34-2.07 (m, 12H), 1.93 (d, J=16.8 Hz, 2H), 1.78 (s, 4H), 1.68-1.41 (m, 12H), 1.30 (d, J=6.8 Hz, 7H), 1.06 (s, 9H), 0.96 (s, 3H) ppm. LC/MS (ESI) m/z 1638.5; [M+H]⁺ calcd for C₈₃H₁₀₈ClF₃N₁₁O₁₀S₄⁺: 1638.68.

(2S,4R)-1-((S)-2-(8-(4-(((R)-4′-chloro-6-((4-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #62) (4.0 mg, 22% yield for two steps). ¹H NMR (600 MHz, Chloroform-d) δ 8.68 (d, J=2.4 Hz, 1H), 8.31 (d, J=5.9 Hz, 1H), 8.06 (d, J=9.1 Hz, 1H), 7.73 (dd, J=9.2, 2.7 Hz, 2H), 7.57-7.47 (m, 1H), 7.42-7.34 (m, 6H), 7.33-7.20 (m, 4H), 6.98 (dd, J=8.3, 2.8 Hz, 2H), 6.81-6.73 (m, 2H), 6.57 (d, J=9.3 Hz, 1H), 5.07 (t, J=7.1 Hz, 1H), 4.72-4.55 (m, 2H), 4.47 (d, J=4.6 Hz, 1H), 4.07-3.96 (m, 1H), 3.86 (s, 1H), 3.69-3.51 (m, 6H), 3.47-3.38 (m, 3H), 3.24 (t, J=5.0 Hz, 4H), 3.13-3.00 (m, 2H), 2.90-2.78 (m, 2H), 2.68-2.46 (m, 10H), 2.42-2.26 (m, 12H), 2.24-2.04 (m, 6H), 1.91 (d, J=17.3 Hz, 1H), 1.86-1.77 (m, 1H), 1.64-1.52 (m, 6H), 1.51-1.40 (m, 4H), 1.31 (s, 5H), 1.03 (d, J=3.0 Hz, 9H), 0.95 (d, J=3.4 Hz, 3H) ppm. LC/MS (ESI) m/z 1628.5; [M+H]⁺ calcd for C₈₁H₁₀₆ClF₃N₁₁O₁₁S₄⁺: 1628.66.

(2S,4R)-1-((2S)-2-(8-(4-(((4R)-6-((4-(4-(((4-(((2R)-4-(2-azabicyclo[2.2.1]heptan-2-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #67) (3.4 mg, 18% yield for two steps). ¹H NMR (600 MHz, CDCl₃) δ 8.69 (s, 1H), 8.30 (s, 1H), 8.02-7.87 (m, 3H), 7.51 (d, J=7.9 Hz, 1H), 7.40 (q, J=8.4 Hz, 4H), 7.38-7.16 (m, 5H), 7.01 (d, J=8.1 Hz, 2H), 6.93-6.72 (m, 4H), 6.40 (d, J=8.9 Hz, 1H), 5.11 (p, J=7.1 Hz, 1H), 4.75 (t, J=8.0 Hz, 1H), 4.62 (d, J=8.9 Hz, 1H), 4.51 (s, 1H), 4.12 (d, J=11.4 Hz, 1H), 4.04 (s, 1H), 3.94 (s, 1H), 3.70-3.53 (m, 3H), 3.44 (s, 2H), 3.22 (s, 4H), 3.16-3.03 (m, 2H), 2.98-2.76 (m, 3H), 2.66-2.49 (m, 8H), 2.49-1.88 (m, 24H), 1.80-1.56 (m, 9H), 1.51-1.42 (m, 4H), 1.38-1.22 (m, 5H), 1.06 (s, 9H), 0.96 (s, 3H) ppm. LC/MS (ESI) m/z 1650.4; [M+H]⁺ calcd for C₈₄H₁₀₈ClF₃N₁₁O₁₀S₄⁺: 1650.68.

(2S,4R)-1-((S)-2-(8-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #59) (13.0 mg, 58% yield for two steps). ¹H NMR (600 MHz, CDCl₃) δ 8.67 (s, 1H), 8.31 (d, J=2.4 Hz, 1H), 8.07 (d, J=9.3 Hz, 1H), 7.73 (dd, J=7.3, 4.9 Hz, 2H), 7.56-7.47 (m, 1H), 7.42-7.33 (m, 7H), 7.26 (s, 4H), 6.98 (dd, J=8.3, 2.3 Hz, 2H), 6.77 (d, J=8.7 Hz, 2H), 6.67-6.54 (m, 2H), 5.08 (q, J=7.2 Hz, 1H), 4.73-4.55 (m, 2H), 4.47 (s, 1H), 4.01 (d, J=11.7 Hz, 1H), 3.88 (s, 1H), 3.76 (t, J=6.1 Hz, 2H), 3.74-3.66 (m, 2H), 3.62-3.52 (m, 3H), 3.42 (t, J=6.5 Hz, 2H), 3.24 (t, J=4.8 Hz, 4H), 3.13-2.99 (m, 2H), 2.84 (s, 2H), 2.79-2.46 (m, 10H), 2.46-2.04 (m, 20H), 1.95-1.85 (m, 3H), 1.73 (dd, J=14.3, 7.2 Hz, 1H), 1.59 (p, J=7.3 Hz, 6H), 1.51-1.41 (m, 4H), 1.36-1.27 (m, 5H), 1.03 (s, 9H), 0.95 (s, 3H) ppm. LC/MS (ESI) m/z 1654.4; [M+H]⁺ calcd for C₈₃H₁₀₈ClF₃N₁₁O₁₁S₄⁺: 1654.67.

(2S,4R)-1-((S)-2-(8-(4-(((R)-6-((4-(4-(((4-(((R)-4-(azepan-1-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #60) (5.7 mg, 31% yield for two steps). ¹H NMR (600 MHz, Chloroform-d) δ 8.69 (s, 1H), 8.29 (d, J=2.2 Hz, 1H), 7.88 (d, J=8.6 Hz, 3H), 7.52 (d, J=7.9 Hz, 1H), 7.44-7.34 (m, 6H), 7.31-7.24 (m, 3H), 7.24-7.19 (m, 1H), 7.04-6.98 (m, 2H), 6.90 (d, J=8.4 Hz, 1H), 6.77 (d, J=8.6 Hz, 2H), 6.68 (d, J=9.2 Hz, 1H), 6.36 (d, J=8.7 Hz, 1H), 5.11 (p, J=7.0 Hz, 1H), 4.75 (t, J=8.0 Hz, 1H), 4.61 (d, J=8.8 8 Hz, 1H), 4.52 (s, 1H), 4.12 (d, J=11.3 Hz, 1H), 3.97 (s, 1H), 3.64-3.53 (m, 3H), 3.44 (d, J=5.2 Hz, 2H), 3.22 (t, J=5.0 Hz, 4H), 3.14-3.01 (m, 5H), 3.00-2.81 (m, 3H), 2.62-2.43 (m, 8H), 2.40 (s, 4H), 2.36-2.08 (m, 12H), 2.01-1.89 (m, 2H), 1.81 (s, 4H), 1.70-1.53 (m, 11H), 1.49 (d, J=7.0 Hz, 4H), 1.38-1.28 (m, 5H), 1.06 (s, 9H), 0.96 (s, 3H) ppm. LC/MS (ESI) m/z 1652.6; [M+H]⁺ calcd for C₈₄H₁₁₀ClF₃N₁₁O₁₀S₄⁺: 1652.70.

(2S,4R)-1-((S)-2-(8-(4-(((R)-4′-chloro-6-((4-(4-(((4-(((R)-4-(5,8-dihydro-1,7-naphthyridin-7(6H)-yl)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #71) (10.0 mg, 51% yield for two steps). ¹H NMR (600 MHz, Chloroform-d) δ 8.70 (s, 1H), 8.39 (d, 1H), 8.33 (d, J=2.3 Hz, 1H), 8.07 (d, 1H), 7.70 (d, J=8.5 Hz, 2H), 7.50 (d, J=7.9 Hz, 1H), 7.47-7.33 (m, 7H), 7.30 (d, J=8.4 Hz, 2H), 7.25-7.17 (m, 3H), 7.12 (dd, J=7.7, 4.7 Hz, 1H), 7.03-6.97 (m, 2H), 6.77 (d, J=8.9 Hz, 2H), 6.59 (d, J=9.3 Hz, 1H), 6.28 (s, 1H), 5.12 (p, J=7.1 Hz, 1H), 4.76 (t, J=8.0 Hz, 1H), 4.64 (d, J=8.8 Hz, 1H), 4.52 (s, 1H), 4.15 (d, J=11.6 Hz, 1H), 3.98 (s, 1H), 3.83 (d, J=15.7 Hz, 1H), 3.68-3.52 (m, 3H), 3.43 (s, 2H), 3.25 (s, 4H), 3.10 (qd, J=13.9, 6.1 Hz, 2H), 2.94-2.78 (m, 4H), 2.71 (t, J=6.0 Hz, 2H), 2.65-2.47 (m, 9H), 2.38 (d, J=17.4 Hz, 4H), 2.32-2.14 (m, 12H), 2.14-2.04 (m, 1H), 1.91 (d, J=17.3 Hz, 1H), 1.82 (dt, J=13.8, 7.0 Hz, 1H), 1.60 (d, J=8.7 Hz, 6H), 1.52-1.40 (m, 4H), 1.29 (d, J=16.2 Hz, 5H), 1.07 (s, 9H), 0.96 (s, 3H) ppm. LC/MS (ESI) m/z 1687.6; [M+H]⁺ calcd for C₈₆H₁₀₇ClF₃N₁₂O₁₀S₄⁺: 1687.68.

### Example 29: Preparation of Compound #61

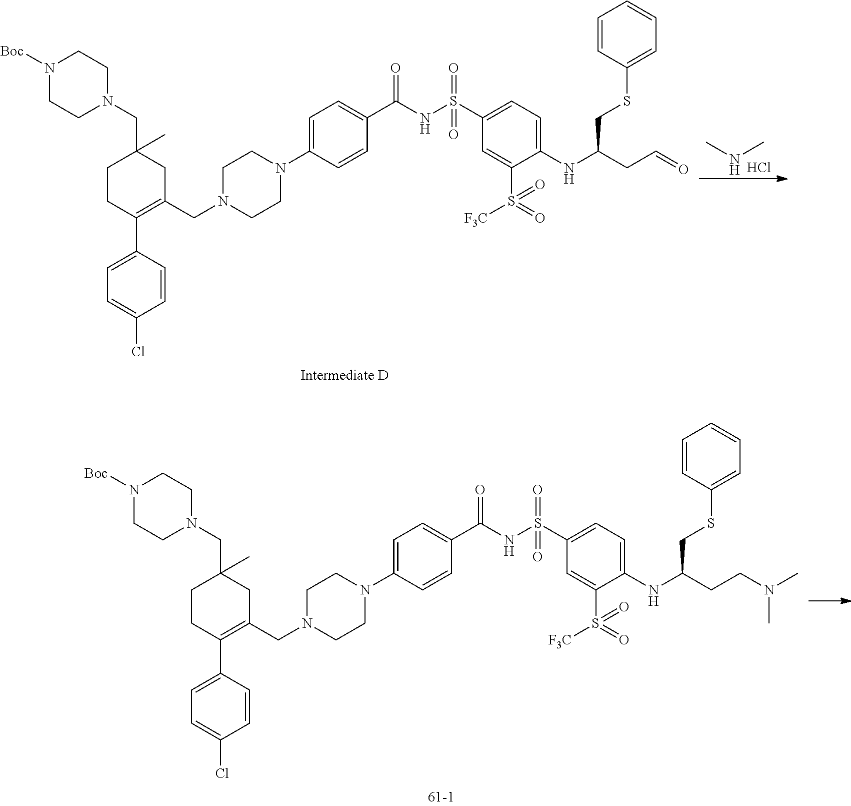

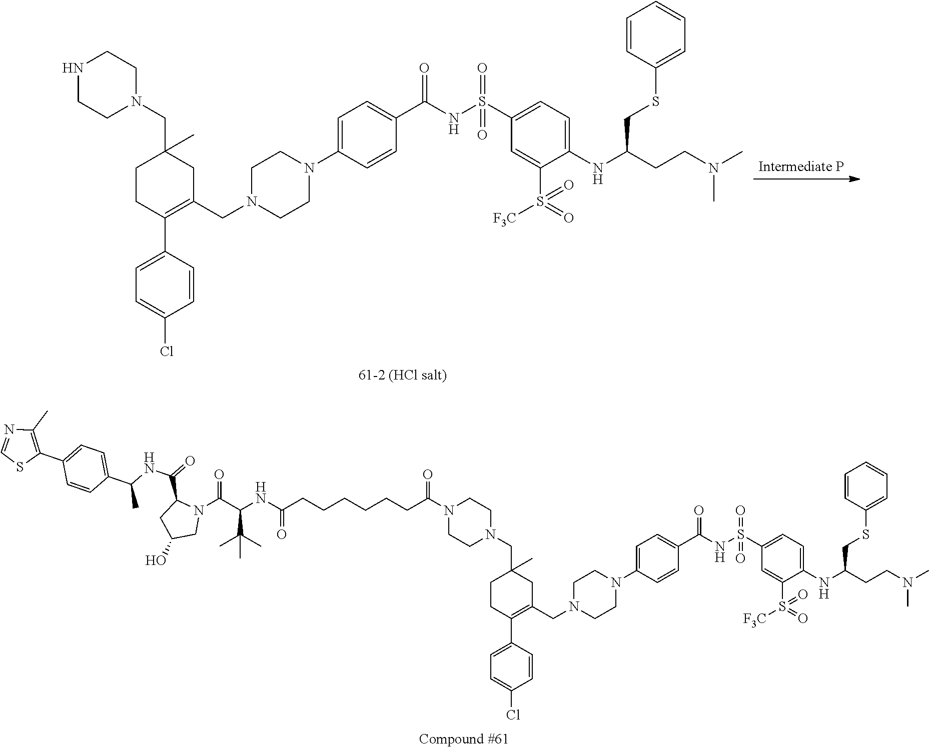 [SMILES: CN(C)CC[C@H](CSc1ccccc1)Nc1ccc(S(=O)(=O)NC(=O)c2ccc(N3CCN(CC4=C(c5ccc(Cl)cc5)CCC(C)(CN5CCNCC5)C4)CC3)cc2)cc1S(=O)(=O)C(F)(F)F ; Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c(S(=O)(=O)C(F)(F)F)c6)cc5)CC4)C3)CC2)C(C)(C)C)cc1]

Compound #61 was prepared by following General Procedure G. tert-butyl 4-((4′-chloro-6-((4-(4-(((4-(((R)-4-(dimethylamino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (61-1) (21 mg, 57% yield). ¹H NMR (600 MHz, Chloroform-d) δ 8.29 (d, J=2.1 8 Hz, 1H), 7.94 (dd, J=9.0, 2.2 Hz, 1H), 7.82 (d, J=8.6 Hz, 2H), 7.37 (d, 2H), 7.30-7.24 (m, 4H), 7.22 (t, J=7.3 Hz, 1H), 7.02-6.95 (m, 2H), 6.76 (d, J=8.7 Hz, 2H), 6.58 (d, J=9.3 Hz, 1H), 3.89 (p, J=5.8 Hz, 1H), 3.45-3.35 (m, 4H), 3.25 (t, J=5.1 Hz, 4H), 3.11-3.00 (m, 2H), 2.87 (s, 2H), 2.75 (dt, J=19.8, 8.2 Hz, 2H), 2.55 (s, 6H), 2.53-2.46 (m, 4H), 2.41 (q, J=5.4 12 Hz, 4H), 2.33-2.10 (m, 6H), 2.00-1.76 (m, 2H), 1.64-1.56 (m, 1H), 1.45 (s, 10H), 0.95 (s, 3H) ppm. LC/MS (ESI) m/z 1116.6; [M+H]⁺ calcd for C₅₄H₇₀ClF₃N₇O₇S₃⁺: 1116.41.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(dimethylamino)-1-(phenylthio)butan-2-yl)amino)-3-((trifluoromethyl)sulfonyl)phenyl)sulfonyl)benzamide hydrochloride (61-2) (22 mg, quantitative yield). LC/MS (ESI) m/z 1016.4; [M+H]⁺ calcd for C₄₉H₆₂ClF₃N₇O₅S₃⁺: 1016.36.

(2S,4R)-1-((2S)-2-(8-(4-((4′-chloro-6-((4-(4-(((4-(((R)-4-(dimethylamino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-2-(3,3-dimethylbutanoyl)-4-hydroxy-N—((S)-1-(4-(4-methylthiazol-5-yl)phenyl)ethyl)pyrrolidine-2-carboxamide (compound #61) (13.6 mg, 45% yield for two steps). ¹H NMR (600 MHz, Chloroform-d) δ 8.67 (s, 1H), 8.27 (d, J=2.1 Hz, 1H), 7.95 (dd, J=9.1, 2.2 Hz, 1H), 7.80 (d, J=8.5 Hz, 2H), 7.56 (dd, J=7.9, 4.2 Hz, 1H), 7.41-7.33 (m, 6H), 7.30-7.23 (m, 4H), 7.24-7.19 (m, 1H), 7.01-6.95 (m, 2H), 6.76 (d, J=8.7 Hz, 2H), 6.67 (dd, J=9.2, 2.9 Hz, 1H), 6.56 (d, J=9.3 Hz, 1H), 5.07 (p, J=7.1 Hz, 1H), 4.64 (t, J=8.2 Hz, 1H), 4.60 (d, 1H), 4.46 (d, J=4.2 Hz, 1H), 4.00 (d, J=11.4 Hz, 1H), 3.85 (p, J=6.3 Hz, 1H), 3.64-3.51 (m, 3H), 3.46-3.37 (m, 2H), 3.21 (t, J=5.1 Hz, 4H), 3.11-2.97 (m, 2H), 2.82 (s, 2H), 2.69-2.47 (m, 9H), 2.46-2.04 (m, 23H), 1.95-1.81 (m, 2H), 1.58 (dq, J=14.6, 7.2 Hz, 6H), 1.51-1.39 (m, 4H), 1.30 (t, J=5.0 Hz, 5H), 1.02 (s, 9H), 0.94 (s, 3H) ppm. LC/MS (ESI) m/z 1598.8; [M+H]⁺ calcd for C₈₀H₁₀₄ClF₃N₁₁O₁₀S₄⁺: 1598.65.

### Example 30: Preparation of Compound #63

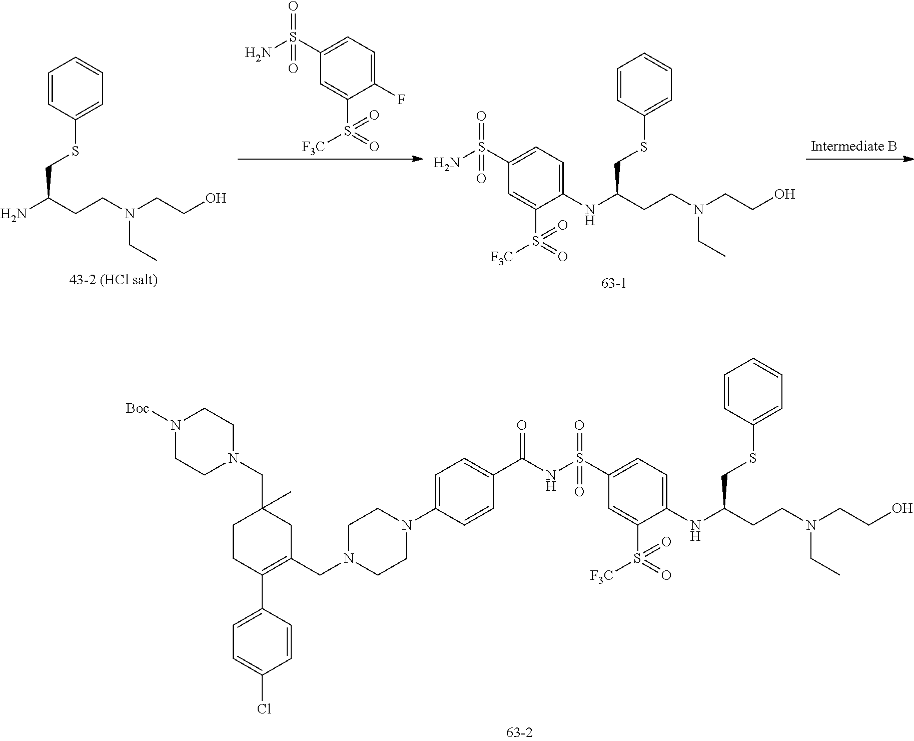 [SMILES: CCN(CCO)CC[C@@H](N)CSc1ccccc1 ; NS(=O)(=O)c1ccc(F)c(S(=O)(=O)C(F)(F)F)c1 ; CCN(CCO)CC[C@H](CSc1ccccc1)Nc1ccc(S(N)(=O)=O)cc1S(=O)(=O)C(F)(F)F ; CCN(CCO)CC[C@H](CSc1ccccc1)Nc1ccc(S(=O)(=O)NC(=O)c2ccc(N3CCN(CC4=C(c5ccc(Cl)cc5)CCC(C)(CN5CCN(C(=O)OC(C)(C)C)CC5)C4)CC3)cc2)cc1S(=O)(=O)C(F)(F)F]

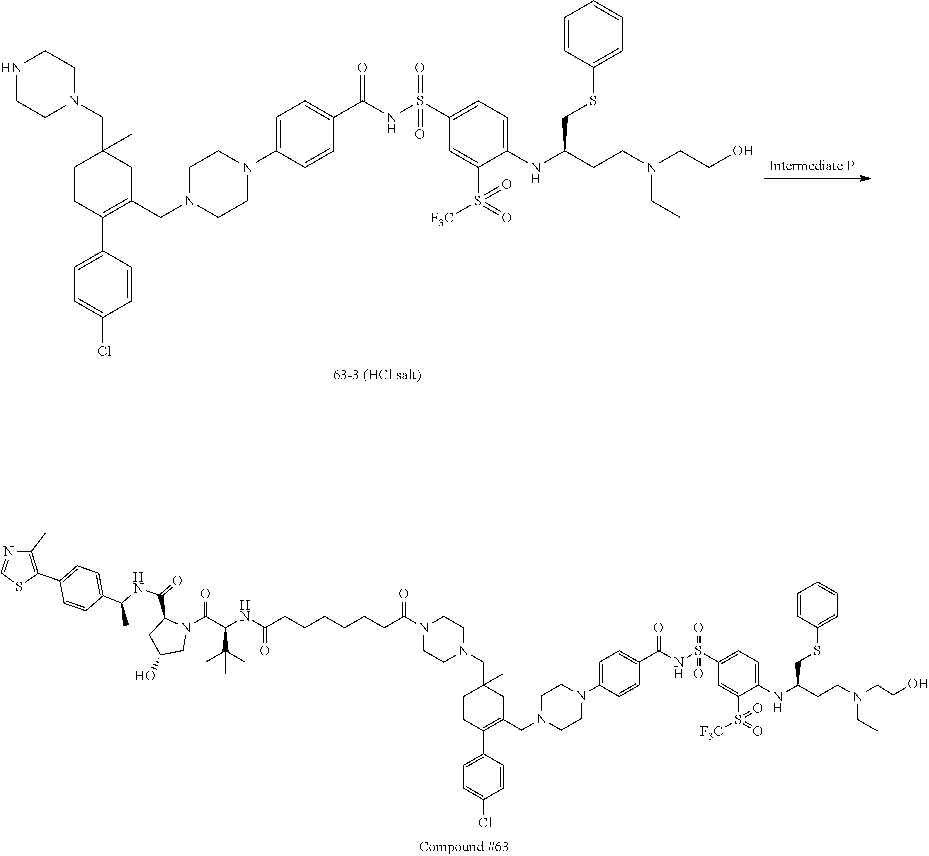 [SMILES: CCN(CCO)CC[C@H](CSc1ccccc1)Nc1ccc(S(=O)(=O)NC(=O)c2ccc(N3CCN(CC4=C(c5ccc(Cl)cc5)CCC(C)(CN5CCNCC5)C4)CC3)cc2)cc1S(=O)(=O)C(F)(F)F ; CCN(CCO)CC[C@H](CSc1ccccc1)Nc1ccc(S(=O)(=O)NC(=O)c2ccc(N3CCN(CC4=C(c5ccc(Cl)cc5)CCC(C)(CN5CCN(C(=O)CCCCCCC(=O)N[C@H](C(=O)N6C[C@H](O)C[C@H]6C(=O)N[C@@H](C)c6ccc(-c7scnc7C)cc6)C(C)(C)C)CC5)C4)CC3)cc2)cc1S(=O)(=O)C(F)(F)F]

Compound #63 was prepared following the general procedure J. (R)-4-((4-(ethyl(2-hydroxyethyl)amino)-1-(phenylthio)butan-2-yl)amino)-3-((trifluoromethyl)sulfonyl)benzenesulfonamide (63-1) (12 mg, 39% yield). ¹H NMR (600 MHz, Chloroform-d) δ 8.22 (q, J=2.5 Hz, 1H), 7.80 (dd, J=9.1, 2.3 Hz, 1H), 7.41 (dt, J=7.2, 2.3 Hz, 2H), 7.36-7.25 (m, 3H), 6.53 (d, J=9.1 Hz, 1H), 3.83 (dq, J=8.5, 4.4 Hz, 1H), 3.56-3.47 (m, 2H), 3.47-3.42 (m, 2H), 3.12-2.99 (m, 2H), 2.70-2.49 (m, 6H), 2.18-2.08 (m, 1H), 1.75-1.65 (m, 1H), 1.01 (t, J=7.1, 3.4 Hz, 3H) ppm. LC/MS (ESI) m/z 556.3; [M+H]⁺ calcd for C₂₁H₂₉F3N₃O₈S₃⁺: 556.12.

tert-butyl 4-((4′-chloro-6-((4-(4-(((4-(((R)-4-(ethyl(2-hydroxyethyl)amino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (63-2) (13 mg, 46% yield). ¹H NMR (600 MHz, Chloroform-d) δ 8.34 (d, J=2.4 Hz, 1H), 8.04 (t, J=8.6 Hz, 1H), 7.77 (t, J=10.4 Hz, 2H), 7.42-7.36 (m, 2H), 7.33-7.22 (m, 4H), 7.03-6.98 (m, 2H), 6.98-6.93 (m, 1H), 6.77 (d, J=8.3 Hz, 2H), 6.55 (d, J=9.3 Hz, 1H), 3.85 (s, 1H), 3.71-3.63 (m, 2H), 3.42 (s, 4H), 3.30-3.19 (m, 4H), 3.11 (dd, J=14.0, 4.5 Hz, 1H), 3.02 (dd, J=14.0, 6.8 4 Hz, 1H), 2.92-2.69 (m, 10H), 2.51 (s, 4H), 2.44-2.26 (m, 4H), 2.26-2.09 (m, 4H), 1.94 (dd, J=17.2, 7.0 Hz, 1H), 1.87-1.77 (m, 1H), 1.69-1.56 (m, 1H), 1.47 (s, 9H), 1.10 (q, J=7.6 Hz, 3H), 0.96 (t, J=6.1 Hz, 5H) ppm. LC/MS (ESI) m/z 1160.7; [M+H]⁺ calcd for C₅₆H₇₄ClF₃N₇O₈S₃⁺: 1160.44.

4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(ethyl(2-hydroxyethyl)amino)-1-(phenylthio)butan-2-yl)amino)-3-((trifluoromethyl)sulfonyl)phenyl)sulfonyl)benzamide hydrochloride (63-3) (15 mg, quantitative yield). LC/MS (ESI) m/z 1060.6; [M+H]⁺ calcd for C₅₁H₆₆ClF₃N₇O₆S₃⁺: 1060.39.

(2S,4R)-1-((2S)-2-(8-(4-((4′-chloro-6-((4-(4-(((4-(((R)-4-(ethyl(2-hydroxyethyl)amino)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #63) (4.3 mg, 23% yield in two steps). ¹H NMR (600 MHz, Chloroform-d) δ 8.68 (s, 1H), 8.29 (s, 1H), 8.03 (d, J=9.0 Hz, 1H), 7.78 (d, J=8.4 Hz, 2H), 7.49 (t, 1H), 7.42-7.33 (m, 6H), 7.31-7.25 (m, 3H), 7.22 (t, J=7.3 Hz, 1H), 7.00 (d, J=8.3 Hz, 2H), 6.75 (d, J=8.6 Hz, 2H), 6.60 (d, J=9.3 Hz, 1H), 6.52 (t, J=8.3 Hz, 1H), 5.09 (p, J=7.1 Hz, 1H), 4.70 (td, J=8.2, 2.3 Hz, 1H), 4.60 (dd, J=9.1, 3.1 Hz, 1H), 4.49 (s, 1H), 4.07 (d, J=11.4 Hz, 1H), 3.87 (s, 1H), 3.66 (s, 2H), 3.59 (dt, J=15.3, 4.9 Hz, 3H), 3.47 26-3.37 (m, 2H), 3.22 (s, 4H), 3.12 (dd, 1H), 3.04 (dd, J=13.9, 6.2 Hz, 1H), 2.93-2.71 (m, 8H), 2.61-2.06 (m, 26H), 1.90 (d, J=17.5 Hz, 1H), 1.82 (d, J=8.4 Hz, 1H), 1.72-1.52 (m, 6H), 1.51-1.38 (m, 4H), 1.37-1.22 (m, 5H), 1.09 (t, J=7.1 Hz, 3H), 1.04 (s, 9H), 0.95 (d, J=4.1 Hz, 3H) ppm. LC/MS (ESI) m/z 1642.4; [M+H]⁺ calcd for C₈₂H₁₀₈ClF₃N₁₁O₁₁S₄⁺: 1642.67.

### Example 31: Preparation of Compound #64

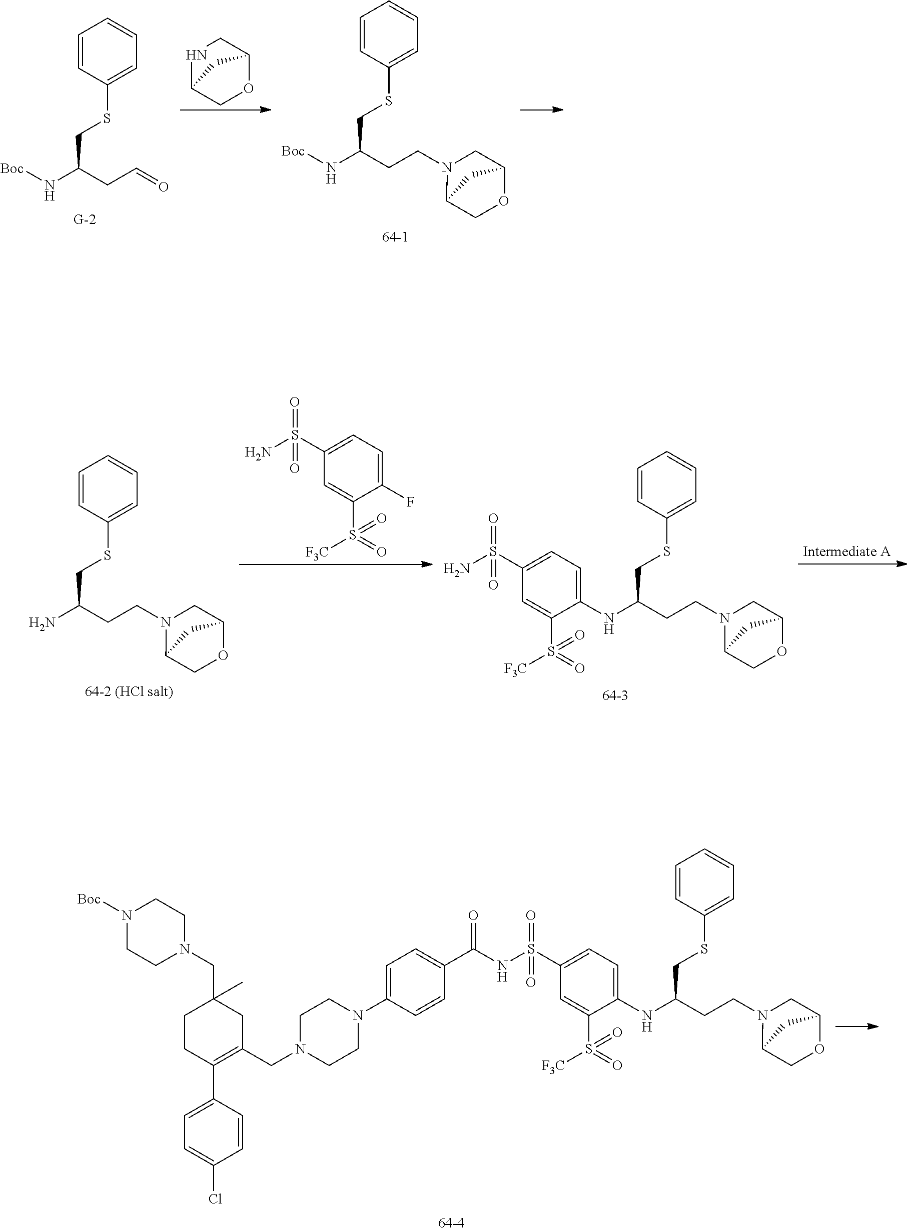

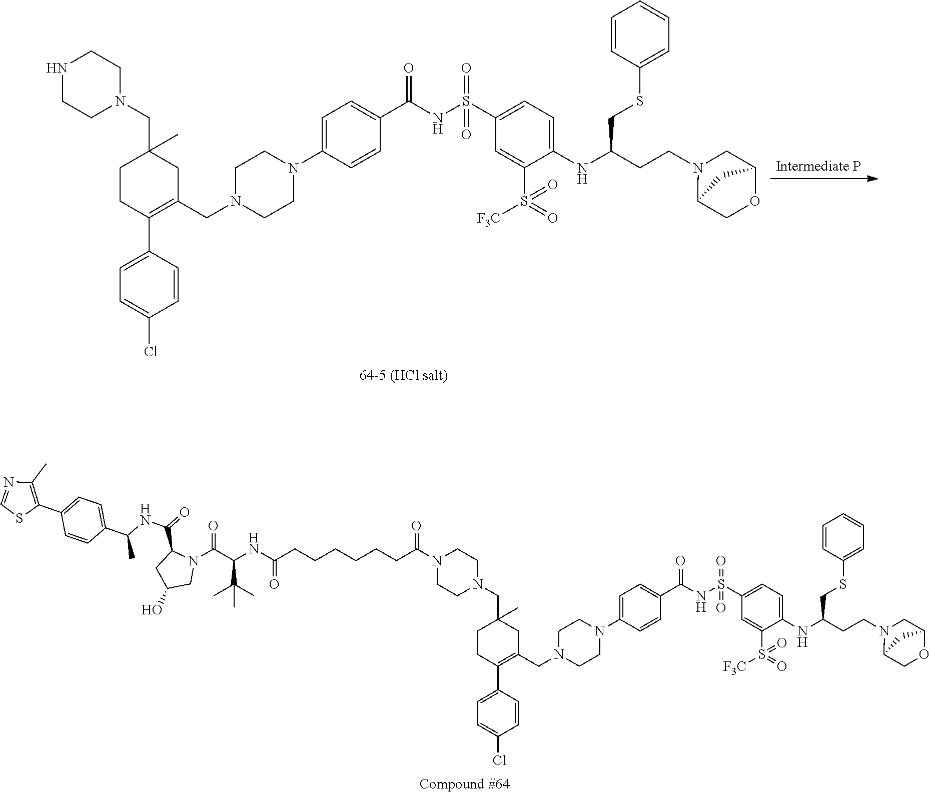 [SMILES: CC1(CN2CCNCC2)CCC(c2ccc(Cl)cc2)=C(CN2CCN(c3ccc(C(=O)NS(=O)(=O)c4ccc(N[C@H](CCN5C[C@@H]6C[C@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@@H]8C[C@H]7CO8)CSc7ccccc7)c(S(=O)(=O)C(F)(F)F)c6)cc5)CC4)C3)CC2)C(C)(C)C)cc1]

Compound #64 was prepared following General Procedure D. tert-butyl ((R)-4-((1S,4S)-2-oxa-5-azabicyclo[2.2.1]heptan-5-yl)-1-(phenylthio)butan-2-yl)carbamate (64-1) (45 mg, 78% yield). ¹H NMR (600 MHz, Chloroform-d) δ 7.41 (d, J=7.6 Hz, 2H), 7.30 (t, J=7.8 Hz, 2H), 7.20 (td, J=7.2, 1.3 Hz, 1H), 5.58 (s, 1H), 4.49 (s, 1H), 4.08 (d, J=9.0 Hz, 1H), 3.91 (s, 1H), 3.82 (s, 1H), 3.69 (d, 1H), 3.28-3.13 (m, 2H), 3.11-2.93 (m, 2H), 2.85-2.69 (m, 2H), 2.08-2.01 (m, 1H), 1.98-1.77 (m, 3H), 1.44 (s, 9H) ppm. LC/MS (ESI) m/z 379.4; [M+H]⁺ calcd for C₂₀H₃₁N₂O₃S⁺: 379.2.

(R)-4-((1S,4S)-2-oxa-5-azabicyclo[2.2.1]heptan-5-yl)-1-(phenylthio)butan-2-amine hydrochloride (64-2) (37 mg, quantitative yield). LC/MS (ESI) m/z 279.1; [M+H]⁺ calcd for C₁₅H₂₃N₂OS⁺: 279.15.

4-(((R)-4-((1S,4S)-2-oxa-5-azabicyclo[2.2.1]heptan-5-yl)-1-(phenylthio)butan-2-yl)amino)-3-((trifluoromethyl)sulfonyl)benzenesulfonamide (64-3) (14 mg, 46% yield in two steps). ¹H NMR (600 MHz, Chloroform-d) δ 8.26 (d, J=2.3 Hz, 1H), 7.83 (dd, J=9.2, 2.4 Hz, 1H), 7.43-7.40 (m, 2H), 7.37-7.32 (m, 2H), 7.24 (d, J=8.5 Hz, 1H), 6.63 (d, J=9.3 Hz, 1H), 4.44 (t, J=2.0 Hz, 1H), 4.01 (d, J=8.1 Hz, 1H), 3.96 (tq, J=8.1, 4.7, 3.9 Hz, 1H), 3.63 (dd, J=8.2, 1.7 Hz, 1H), 3.48 (s, 1H), 3.15-3.03 (m, 2H), 2.99 (dd, J=10.1, 1.7 Hz, 1H), 2.82-2.75 (m, 1H), 2.70-2.63 (m, 1H), 2.51 (dd, J=10.1, 1.5 Hz, 1H), 2.06-1.99 (m, 1H), 1.93 (dd, J=10.1, 2.2 Hz, 1H), 1.86-1.80 (m, 1H), 1.78 (dd, J=9.9, 2.9, 1.5 Hz, 1H) ppm. LC/MS (ESI) m/z 566.0; [M+H]⁺ calcd for C₂₂H₂₇F₃N₃O₅S₃⁺: 566.11.

tert-butyl 4-((6-((4-(4-(((4-(((R)-4-((1S,4S)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (64-4) (18 mg, 64% yield). ¹H NMR (600 MHz, Chloroform-d) δ 8.37 (dt, J=4.8, 2.5 Hz, 1H), 8.12-8.05 (m, 1H), 7.74-7.65 (m, 2H), 7.41-7.37 (m, 2H), 7.35-7.20 (m, 7H), 7.02-6.98 (m, 2H), 6.79 (d, J=7.1 Hz, 2H), 6.60 (d, J=8.5 Hz, 1H), 4.44 (s, 1H), 4.02 (d, J=8.1 Hz, 1H), 3.97 (s, 1H), 3.62 (d, J=7.4 Hz, 1H), 3.58-3.47 (m, 1H), 3.42 (d, J=5.0 Hz, 4H), 3.28 (t, J=5.1 Hz, 4H), 3.14-3.01 (m, 3H), 2.89-2.74 (m, 3H), 2.67 (s, 1H), 2.56-2.47 (m, 5H), 2.43-2.27 (m, 5H), 2.27-2.19 (m, 3H), 2.15 (d, J=17.4 Hz, 1H), 2.05-1.89 (m, 2H), 1.87-1.74 (m, 2H), 1.62 (dt, J=14.1, 7.5 Hz, 1H), 1.47 (s, 10H), 0.96 (d, J=3.7 Hz, 3H) ppm. LC/MS (ESI) m/z 1170.6; [M+H]⁺ calcd for C₅₇H₇₂ClF₃N₇O₈S₃⁺: 1170.42.

N-((4-(((R)-4-((1S,4S)-2-oxa-5-azabicyclo[2.2.1]heptan-5-yl)-1-(phenylthio)butan-2-yl)amino)-3-((trifluoromethyl)sulfonyl)phenyl)sulfonyl)-4-(4-((4′-chloro-4-methyl-4-(piperazin-1-ylmethyl)-3,4,5,6-tetrahydro-[1,1′-biphenyl]-2-yl)methyl)piperazin-1-yl)benzamide hydrochloride (64-5) (20 mg, quantitative yield). LC/MS (ESI) m/z 1070.6; [M+H]⁺ calcd for C₅₂H₆₄ClF₃N₇O₆S₃⁺: 1070.37

(2S,4R)-1-((2S)-2-(8-(4-((6-((4-(4-(((4-(((R)-4-((1S,4S)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #64) (17.3 mg, 68% yield in two steps). ¹H NMR (600 MHz, Chloroform-d) δ 8.69 (s, 1H), 8.32 (d, J=2.4 Hz, 1H), 8.05 (d, J=9.2 Hz, 1H), 7.82-7.73 (m, 2H), 7.49 (dd, J=18.8, 7.7 Hz, 1H), 7.43-7.35 (m, 6H), 7.33-7.22 (m, 5H), 7.01 (d, J=8.0 Hz, 2H), 6.75 (s, 2H), 6.60 (d, J=8.6 Hz, 1H), 6.34 (s, 1H), 5.10 (p, 1H), 4.78-4.69 (m, 1H), 4.62 (dd, J=8.9, 5.1 Hz, 1H), 4.51 (s, 1H), 4.43 (s, 1H), 4.11 (d, J=11.1 Hz, 1H), 4.01 (d, J=8.3 Hz, 1H), 3.95 (s, 1H), 3.63-3.49 (m, 4H), 3.44 (s, 2H), 3.31-3.14 (m, 4H), 3.15-3.01 (m, 2H), 3.01-2.73 (m, 3H), 2.68-2.36 (m, 14H), 2.34-2.14 (m, 12H), 2.11 (dd, J=13.5, 8.3 Hz, 1H), 2.02-1.87 (m, 3H), 1.85-1.73 (m, 2H), 1.66-1.52 (m, 6H), 1.52-1.41 (m, 4H), 1.38-1.23 (m, 5H), 1.06 (s, 9H), 0.97 (s, 3H) ppm. LC/MS (ESI) m/z 1652.8; [M+H]⁺ calcd for C₈₃H₁₀₆ClF₃N₁₁O₁₁S₄⁺: 1652.66.

### Example 32: Preparation of Compound #65

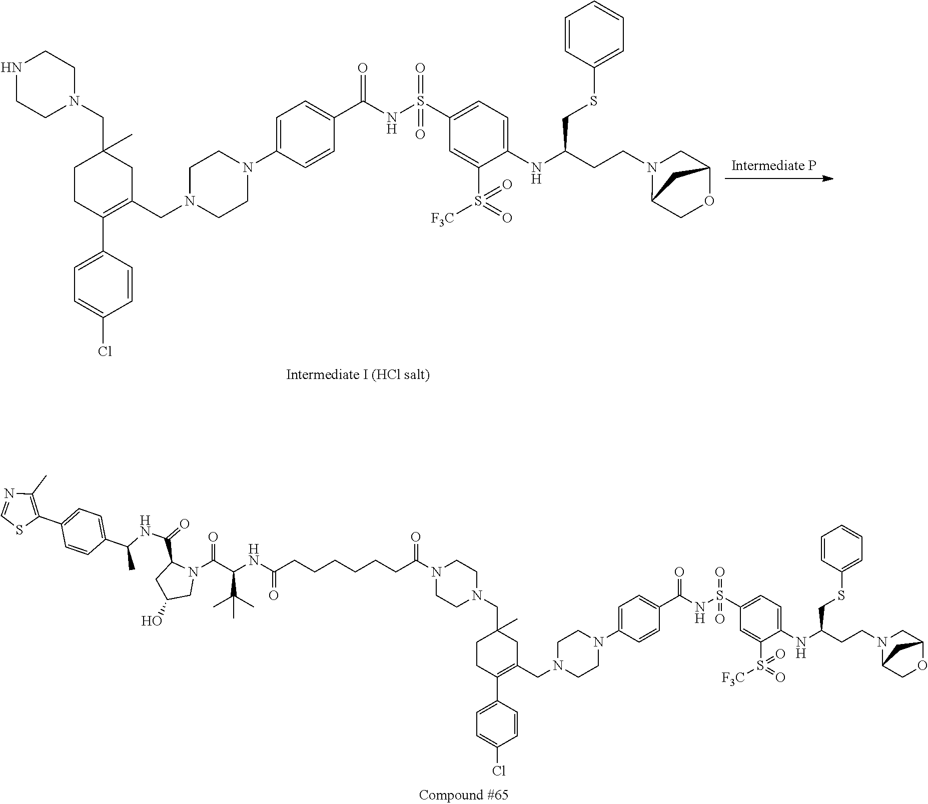 [SMILES: CC1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c(S(=O)(=O)C(F)(F)F)c6)cc5)CC4)C3)CC2)C(C)(C)C)cc1]

Compound #65 was prepared by following General Procedure H. (2S,4R)-1-((2S)-2-(8-(4-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #65) (14.9 mg, 49% yield). ¹H NMR (600 MHz, Chloroform-d) δ 8.69 (d, J=1.5 Hz, 1H), 8.32 (d, J=4.6 Hz, 1H), 8.06 (s, 1H), 7.85-7.71 (m, 2H), 7.50 (dd, 1H), 7.43-7.35 (m, 6H), 7.35-7.20 (m, 5H), 7.01 (d, J=8.0 Hz, 2H), 6.76 (s, 2H), 6.59 (d, J=9.2 Hz, 1H), 6.32 (s, 1H), 5.15-5.06 (m, 1H), 4.78-4.68 (m, 1H), 4.60 (t, J=8.6 Hz, 1H), 4.51 (s, 1H), 4.43 (s, 1H), 4.13 (s, 1H), 4.02-3.89 (m, 2H), 3.66-3.51 (m, 4H), 3.43 (s, 2H), 3.31-3.16 (m, 4H), 3.14-3.01 (m, 2H), 2.99-2.84 (m, 2H), 2.80-2.34 (m, 14H), 2.34-2.14 (m, 12H), 2.11 (dd, 1H), 2.02 (s, 1H), 1.97-1.84 (m, 2H), 1.82-1.71 (m, 2H), 1.68-1.54 (m, 6H), 1.54-1.40 (m, 4H), 1.40-1.23 (m, 5H), 1.06 (s, 9H), 0.97 (s, 3H) ppm. LC/MS (ESI) m/z 1652.5; [M+H]⁺ calcd for C₈₃H₁₀₆ClF₃N₁₁O₁₁S₄⁺: 1652.66.

### Example 33: Preparation of Compound #66

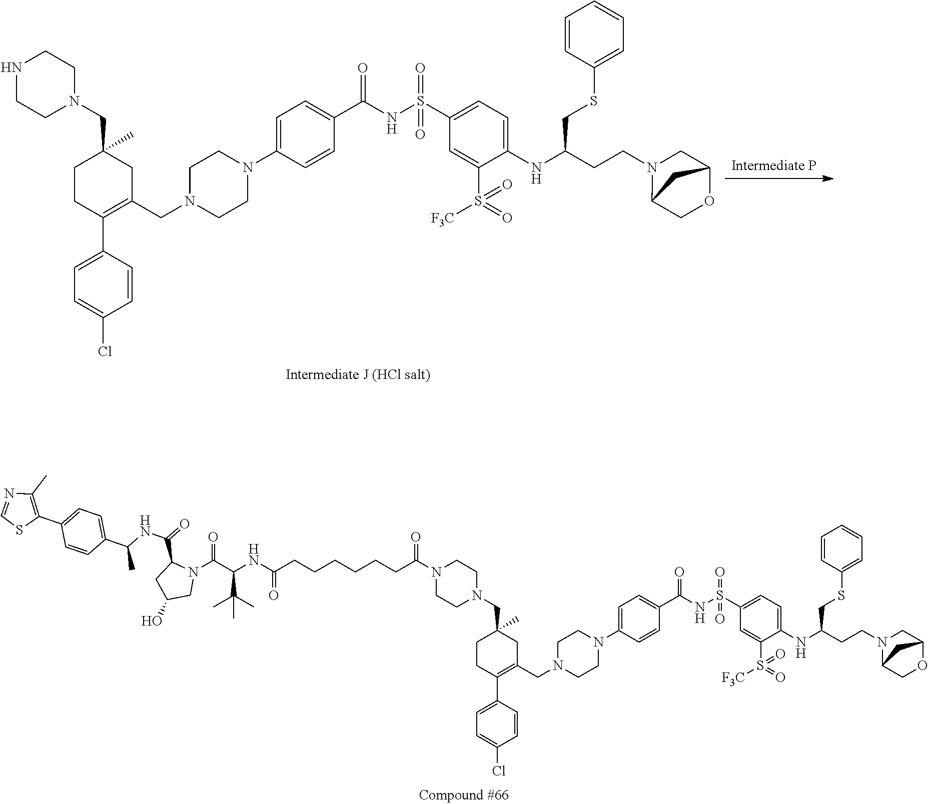 [SMILES: C[C@@]1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7C[C@H]8C[C@@H]7CO8)CSc7ccccc7)c(S(=O)(=O)C(F)(F)F)c6)cc5)CC4)C3)CC2)C(C)(C)C)cc1]

Compound #66 was prepared by following General Procedure H. (2S,4R)-1-((S)-2-(8-(4-(((R)-6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #66) (4.7 mg, 11% yield). ¹H NMR (600 MHz, Chloroform-d) δ 8.69 (d, J=1.9 Hz, 1H), 8.33 (t, J=2.2 Hz, 1H), 8.08 (d, J=9.2 Hz, 1H), 7.74 (d, J=8.6 Hz, 2H), 7.49 (s, 1H), 7.43-7.35 (m, 6H), 7.34-7.23 (m, 5H), 7.00 (dd, J=8.5, 2.1 Hz, 2H), 6.77 (d, J=8.9 Hz, 2H), 6.58 (d, J=9.3 Hz, 1H), 6.36 (s, 1H), 5.10 (p, J=7.5 Hz, 1H), 4.73 (s, 1H), 4.61 (d, J=8.8 Hz, 1H), 4.50 (s, 1H), 4.42 (s, 1H), 4.12 (s, 1H), 3.98 (d, J=8.1 Hz, 1H), 3.93 (s, 1H), 3.67-3.52 (m, 3H), 3.53-3.47 (m, 1H), 3.43 (t, J=5.2 Hz, 2H), 3.24 (t, J=5.5 Hz, 4H), 3.15-3.00 (m, 2H), 2.93-2.81 (m, 3H), 2.78-2.69 (m, 1H), 2.66 (dt, J=12.2, 6.1 Hz, 1H), 2.59 (d, J=10.1 Hz, 2H), 2.52 (s, 5H), 2.48-2.33 (m, 4H), 2.33-2.14 (m, 12H), 2.11 (dd, J=13.6, 8.3 Hz, 1H), 2.07-1.98 (m, 1H), 1.89 (dd, 2H), 1.80-1.68 (m, 2H), 1.68-1.54 (m, 6H), 1.52-1.40 (m, 4H), 1.30 (s, 5H), 1.05 (s, 9H), 0.96 (s, 3H) ppm. LC/MS (ESI) m/z 1652.4; [M+H]⁺ calcd for C₈₃H₁₀₆ClF₃N₁₁O₁₁S₄⁺: 1652.66.

### Example 34: Preparation of Compound #68

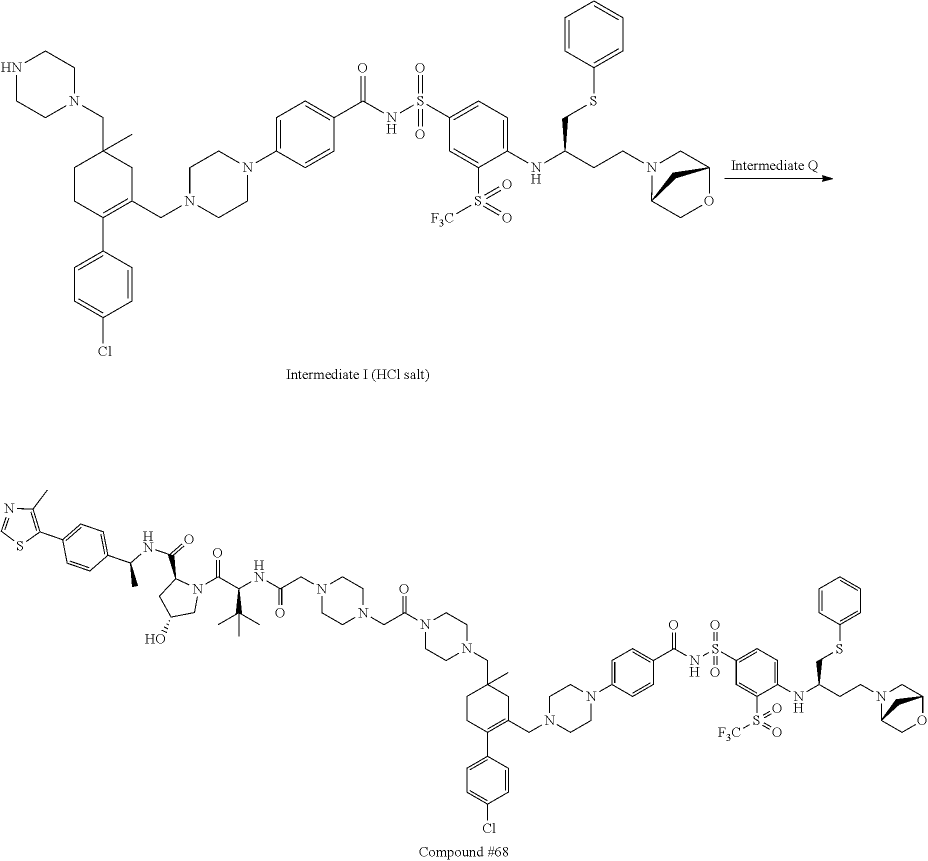 [SMILES: CC1(CN2CCNCC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CN2CCN(CC(=O)N3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1]

Compound #68 was prepared by following General Procedure H. (2S,4R)-1-((2S)-2-(2-(4-(2-(4-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-2-oxoethyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #68) (7.9 mg, 35% yield). ¹H NMR (600 MHz, Chloroform-d) δ 8.69 (s, 1H), 8.32 (s, 1H), 8.08 (s, 1H), 7.75 (dd, J=9.0, 4.7 Hz, 2H), 7.44-7.35 (m, 7H), 7.34-7.22 (m, 5H), 7.02-6.96 (m, 2H), 6.79 (d, 2H), 6.58 (d, J=9.3 Hz, 1H), 5.09 (p, J=7.5 Hz, 1H), 4.72 (s, 1H), 4.61-4.47 (m, 2H), 4.41 (s, 1H), 4.16 (s, 1H), 3.97 (d, J=8.0 Hz, 1H), 3.92 (s, 1H), 3.64-3.53 (m, 5H), 3.45 (s, 1H), 3.33-2.77 (m, 14H), 2.75-2.19 (m, 26H), 2.10 (dd, 1H), 2.06-1.97 (m, 1H), 1.93-1.82 (m, 2H), 1.76-1.59 (m, 4H), 1.54-1.39 (m, 4H), 1.39-1.28 (m, 5H), 1.06 (s, 9H), 0.96 (s, 3H) ppm. LC/MS (ESI) m/z 1680.5; [M+H]⁺ calcd for C₈₃H₁₀₆ClF₃N₁₃O₁₁S₄⁺: 1680.67.

### Example 35: Preparation of Compound #69

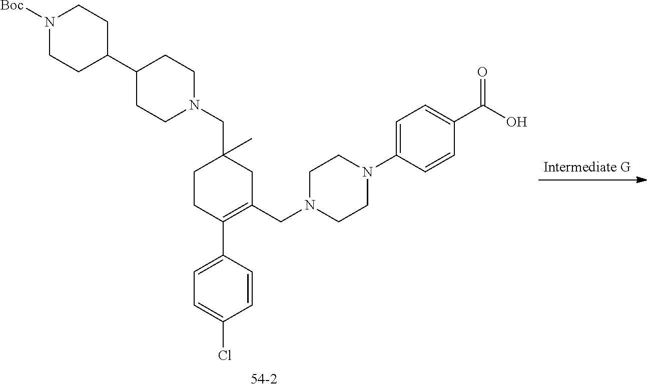 [SMILES: CC1(CN2CCC(C3CCN(C(=O)OC(C)(C)C)CC3)CC2)CCC(c2ccc(Cl)cc2)=C(CN2CCN(c3ccc(C(=O)O)cc3)CC2)C1]

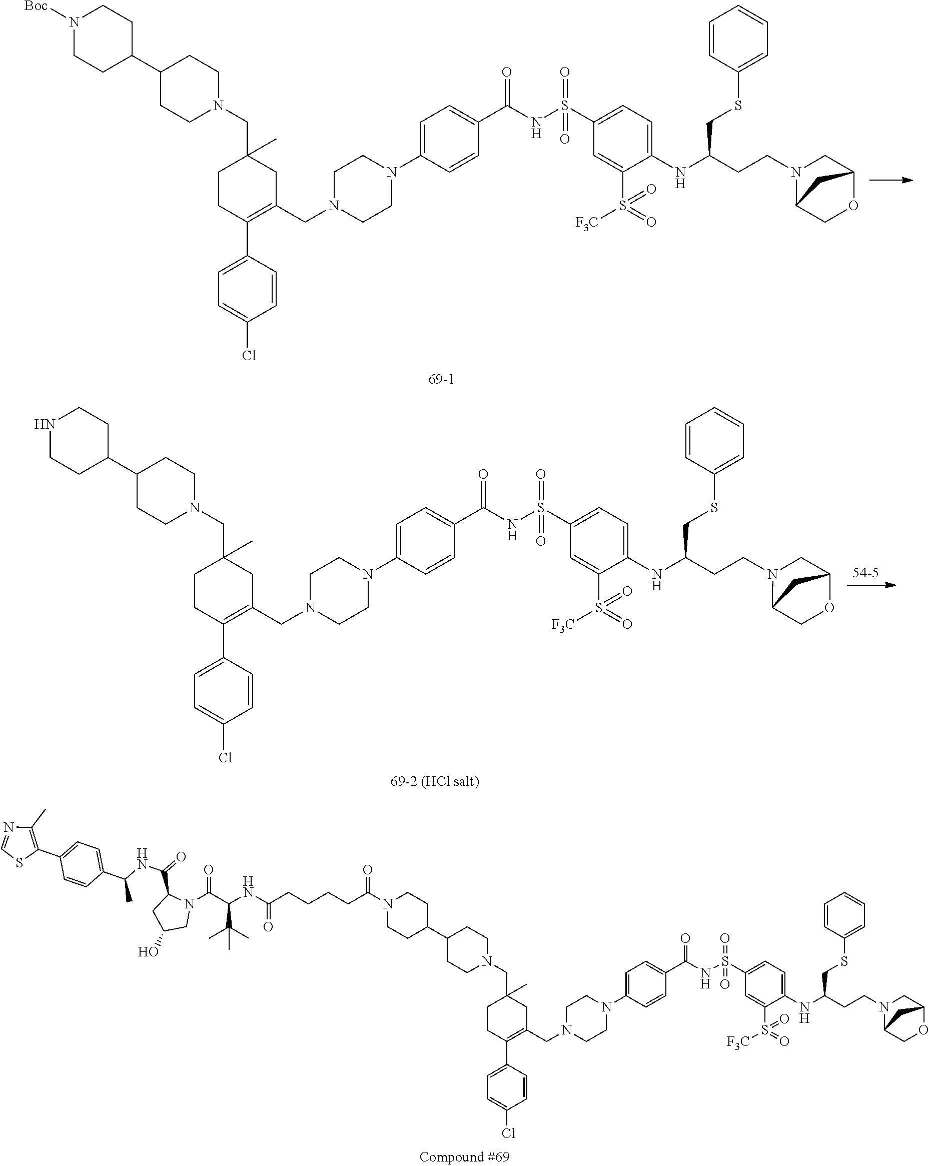

Compound #69 was prepared by following General Procedure E. tert-butyl 1′-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidine]-1-carboxylate (69-1) (36 mg, 68% yield). ¹H NMR (600 MHz, Chloroform-d) δ 8.35 (d, J=2.2 16 Hz, 1H), 8.02 (d, J=9.1 Hz, 1H), 7.86 (d, J=8.4 Hz, 2H), 7.42-7.37 (m, 2H), 7.34-7.22 (m, 5H), 6.99 (d, J=8.1 Hz, 2H), 6.73 (d, J=8.3 Hz, 2H), 6.46 (d, J=9.2 Hz, 1H), 4.44 (s, 1H), 4.09 (s, 2H), 4.00 (d, J=8.2 Hz, 1H), 3.84 (s, 1H), 3.65 (dd, J=8.2, 1.8 Hz, 1H), 3.55 (s, 1H), 3.20 (s, 5H), 3.14-2.99 (m, 2H), 2.97-2.82 (m, 2H), 2.74 (dt, J=13.4, 7.0 Hz, 1H), 2.70-2.11 (m, 18H), 2.10-1.99 (m, 1H), 1.90 (d, J=10.0 Hz, 1H), 1.79 (d, J=10.0 Hz, 1H), 1.76-1.60 (m, 5H), 1.59-1.41 (m, 10H), 1.38-1.17 (m, 5H), 1.16-0.99 (m, 6H) ppm. LC/MS (ESI) m/z 1252.3; [M+H]⁺ calcd for C₆₃H₈₂ClF₃N₇O₈S₃⁺: 1252.50.

N-((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)-4-(4-((4-([4,4′-bipiperidin]-1-ylmethyl)-4′-chloro-4-methyl-3,4,5,6-tetrahydro-[1,1′-biphenyl]-2-yl)methyl)piperazin-1-yl)benzamide hydrochloride (69-2) (40 mg, quantitative yield). LC/MS (ESI) m/z 1152.3; [M+H]⁺ calcd for C₅₈H₇₄ClF₃N₇O₆S₃⁺: 1152.45.

(2S,4R)-1-((2S)-2-(6-(1′-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (compound #69) (10.5 mg, 49% yield). ¹H NMR (600 MHz, Chloroform-d) δ 8.67 (s, 1H), 8.64 (t, J=2.2 Hz, 1H), 7.86 (d, J=8.5 Hz, 2H), 7.79 (d, J=9.1 Hz, 1H), 7.52 (d, J=7.1 Hz, 1H), 7.38 (q, J=8.2 Hz, 4H), 7.31-7.25 (m, 4H), 7.22-7.12 (m, 3H), 6.99 (d, J=8.4 Hz, 2H), 6.74 (dd, J=15.0, 9.0 Hz, 3H), 6.59 (s, 1H), 5.07 (q, J=7.2 Hz, 1H), 4.70-4.63 (m, 1H), 4.60 (d, J=9.1 Hz, 1H), 4.47 (s, 1H), 4.12-3.97 (m, 2H), 3.62-3.52 (m, 3H), 3.48-3.39 (m, 2H), 3.20 (t, J=5.0 Hz, 4H), 3.16-3.04 (m, 7H), 3.00 (s, 1H), 2.82 (s, 2H), 2.60-2.45 (m, 7H), 2.44-2.16 (m, 14H), 2.15-1.73 (m, 12H), 1.69-1.54 (m, 8H), 1.52-1.41 (m, 4H), 1.36-1.27 (m, 5H), 1.03 (s, 9H), 0.95 (s, 3H) ppm. LC/MS (ESI) m/z 1706.9; [M+H]⁺ calcd for C₈₇H₁₁₂ClF₃N₁₁O₁₁S₄⁺: 1706.71.

### Example 36: Preparation of Compound #70

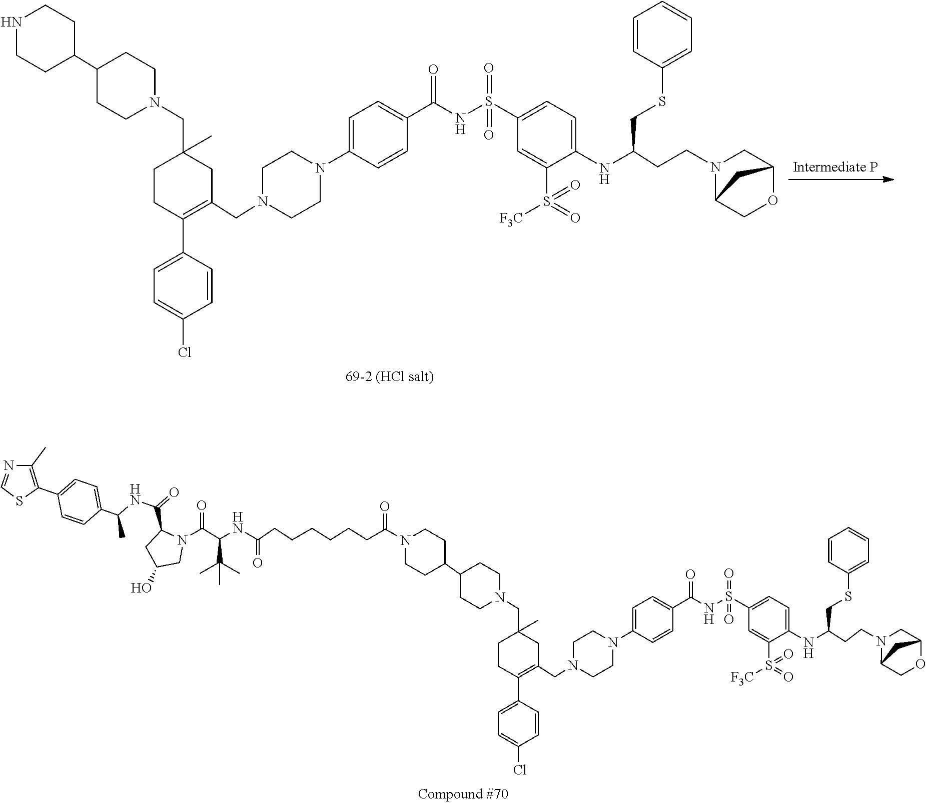 [SMILES: CC1(CN2CCC(C3CCNCC3)CC2)CCC(c2ccc(Cl)cc2)=C(CN2CCN(c3ccc(C(=O)NS(=O)(=O)c4ccc(N[C@H](CCN5C[C@H]6C[C@@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)CCCCCCC(=O)N2CCC(C3CCN(CC4(C)CCC(c5ccc(Cl)cc5)=C(CN5CCN(c6ccc(C(=O)NS(=O)(=O)c7ccc(N[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1]

Compound #70 was prepared by following General Procedure H. (2S,4R)-1-((2S)-2-(8-(1′-((6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #70) (2.6 mg, 12% yield). ¹H NMR (600 MHz, Chloroform-d) δ 8.68 (s, 1H), 8.60 (d, J=10.6 Hz, 1H), 7.89 (d, J=8.3 Hz, 2H), 7.80 (dd, J=21.1, 8.8 Hz, 1H), 7.52 (d, J=7.8 Hz, 1H), 7.39 (q, J=8.0 Hz, 4H), 7.27 (d, J=7.6 Hz, 4H), 7.21-7.11 (m, 3H), 7.00 (d, J=8.0 Hz, 2H), 6.76 (t, J=11.0 Hz, 3H), 6.57 (d, J=9.1 2 Hz, 1H), 5.08 (t, J=7.2 Hz, 1H), 4.68 (t, J=7.9 Hz, 1H), 4.61 (d, J=8.9 Hz, 1H), 4.48 (s, 1H), 4.19-3.90 (m, 3H), 3.63-3.51 (m, 4H), 3.43 (d, J=4.3 Hz, 2H), 3.34-2.99 (m, 8H), 2.94-2.77 (m, 3H), 2.67-2.47 (m, 8H), 2.43-2.04 (m, 22H), 1.93 (d, J=17.3 Hz, 1H), 1.83-1.40 (m, 18H), 1.36-1.27 (m, 5H), 1.03 (s, 9H), 0.95 (s, 3H) ppm. LC/MS (ESI) m/z 1734.6; [M+H]+ calcd for Cs₉H₁₁₆ClF₃N₁₁O₁₁S₄⁺: 1734.74.

### Example 37: Preparation of Compound #72

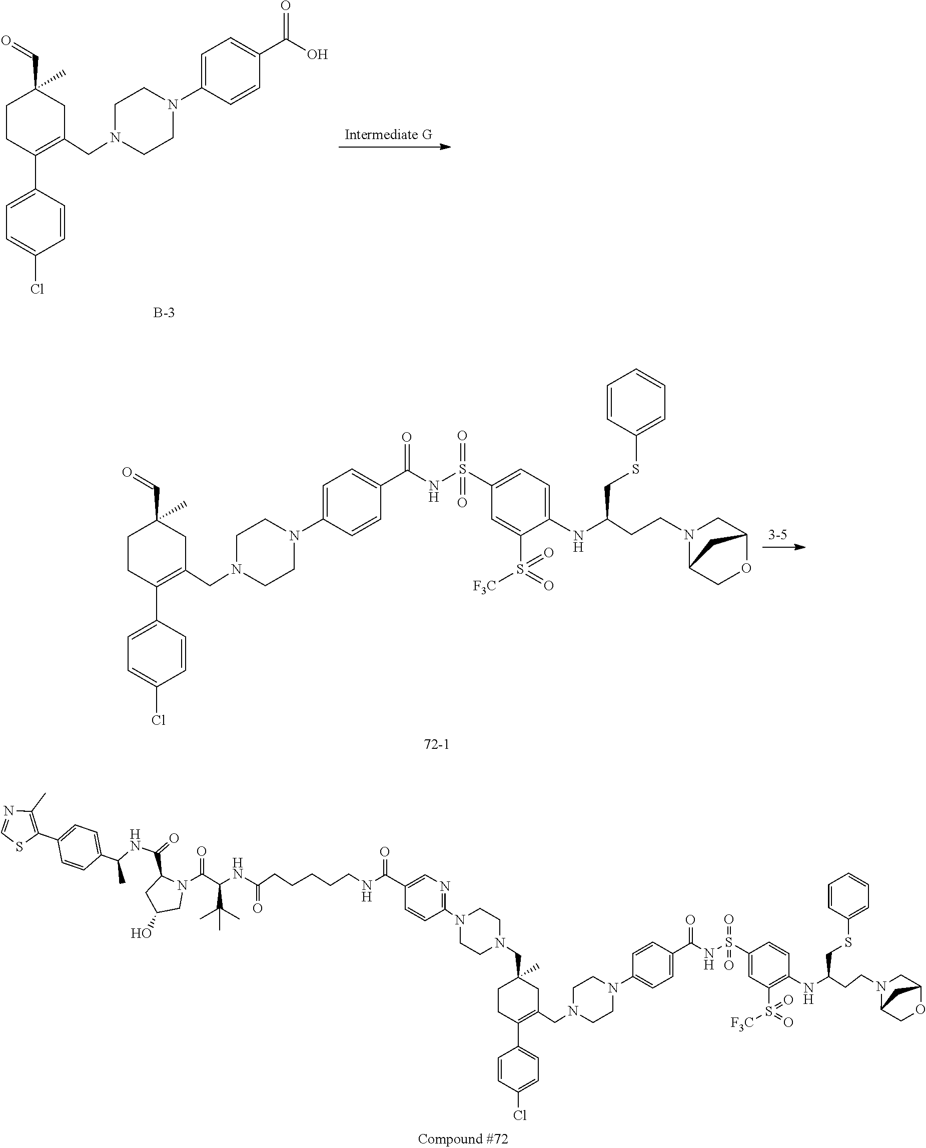

Preparation of N-((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)-4-(4-(((R)-4′-chloro-4-formyl-4-methyl-3,4,5,6-tetrahydro-[1,1′-biphenyl]-2-yl)methyl)piperazin-1-yl)benzamide (72-1): Intermediate G (100 mg, 0.18 mmol) was added to a mixture of B-3 (96 mg, 0.21 mmol), EDC (170 mg, 0.88 mmol), and DMAP (108 mg, 0.88 mmol) in DCM (3 mL). The resulting mixture was stirred at room temperature overnight. The reaction mixture was washed with sat. aq. NH₄Cl, dried over Na₂SO₄, filtered, and concentrated. The residue was purified by flash column chromatography (0% to 5% of MeOH in DCM) to afford 72-1 (114 mg, 64% yield). ¹H NMR (600 MHz, CDCl₃) δ 9.54 (s, 1H), 8.37 (d, J=2.3 Hz, 1H), 8.08 (dd, J=9.2, 2.3 Hz, 1H), 7.70 (d, J=8.6 Hz, 2H), 7.42-7.37 (m, 2H), 7.35-7.24 (m, 5H), 7.14 (d, J=8.3 Hz, 1H), 6.99-6.94 (m, 2H), 6.81 (d, 2H), 6.60 (d, J=9.4 Hz, 1H), 4.44 (t, J=2.0 Hz, 1H), 4.00 (d, J=8.1 Hz, 1H), 3.98-3.92 (m, 1H), 3.65 (dd, J=8.1, 1.8 Hz, 1H), 3.54 (s, 1H), 3.31 (t, J=5.2 Hz, 4H), 3.11 (dd, J=13.9, 5.0 Hz, 1H), 3.05 (dd, J=13.9, 6.9 Hz, 1H), 2.93 (d, J=10.1 2 Hz, 1H), 2.91-2.83 (m, 2H), 2.80-2.73 (m, 1H), 2.72-2.65 (m, 2H), 2.62 (d, J=10.1 Hz, 1H), 2.44 (dt, J=10.4, 4.0 Hz, 2H), 2.37 (dt, J=10.7, 5.1 Hz, 2H), 2.31 (d, J=5.9 Hz, 2H), 2.08 4-1.97 (m, 3H), 1.90 (d, J=10.8 Hz, 1H), 1.82-1.72 (m, 2H), 1.64 (dt, J=14.2, 7.3 Hz, 1H), 1.16 (s, 3H) ppm. LC/MS (ESI) m/z 1000.4; [M+H]⁺ calcd for C₄₈H₅₄ClF₃N5O₇S₃⁺: 1000.28.

Preparation of 6-(4-(((R)-6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)nicotinamide (compound #72): General Procedure K was applied to obtain compound #72 (4.1 mg, 16% yield). ¹H NMR (600 MHz, CDCl₃) δ 8.68 (s, 1H), 8.59 (d, J=2.5 Hz, 1H), 8.33 (d, J=2.3 Hz, 1H), 8.10 (dd, J=9.2, 2.3 Hz, 1H), 7.94 (dd, J=9.0, 2.5 Hz, 1H), 7.70 (dd, J=9.3, 3.6 Hz, 2H), 7.43 (d, J=7.8 Hz, 1H), 7.39 (td, J=6.6, 3.1 Hz, 4H), 7.35 (d, J=8.3 Hz, 2H), 7.34-7.22 (m, 5H), 7.03-6.96 (m, 2H), 6.81-6.74 (m, 2H), 6.63-6.55 (m, 3H), 5.07 (p, J=7.1 Hz, 1H), 4.65-4.58 (m, 2H), 4.47 (s, 1H), 4.41 (t, J=2.0 Hz, 1H), 4.05 (d, J=11.4 Hz, 1H), 3.97 (d, J=8.0 Hz, 1H), 3.94-3.90 (m, 1H), 3.65-3.53 (m, 6H), 3.46-3.39 (m, 4H), 3.30-3.20 (m, 4H), 3.10 (dd, J=13.6, 4.8 Hz, 1H), 3.05 (dd, J=13.9, 6.9 Hz, 1H), 2.91-2.81 (m, 3H), 2.73-2.59 (m, 6H), 2.51 (s, 4H), 2.45-2.19 (m, 18H), 2.08 (dd, J=13.5, 8.0 Hz, 1H), 2.05-1.98 (m, 1H), 1.92 (d, J=17.2 Hz, 1H), 1.85 (d, J=9.9 Hz, 1H), 1.77-1.53 (m, 4H), 1.46 (d, J=6.9 Hz, 3H), 1.44-1.36 (m, 1H), 1.04 (s, 9H), 0.97 (s, 3H) ppm. LC/MS (ESI) m/z 1730.5; [M+H]⁺ calcd for C₈₇H₁₀₈ClF₃N₁₃O₁₁S₄⁺: 1730.68.

### Example 38: Preparation of Compounds #73-74

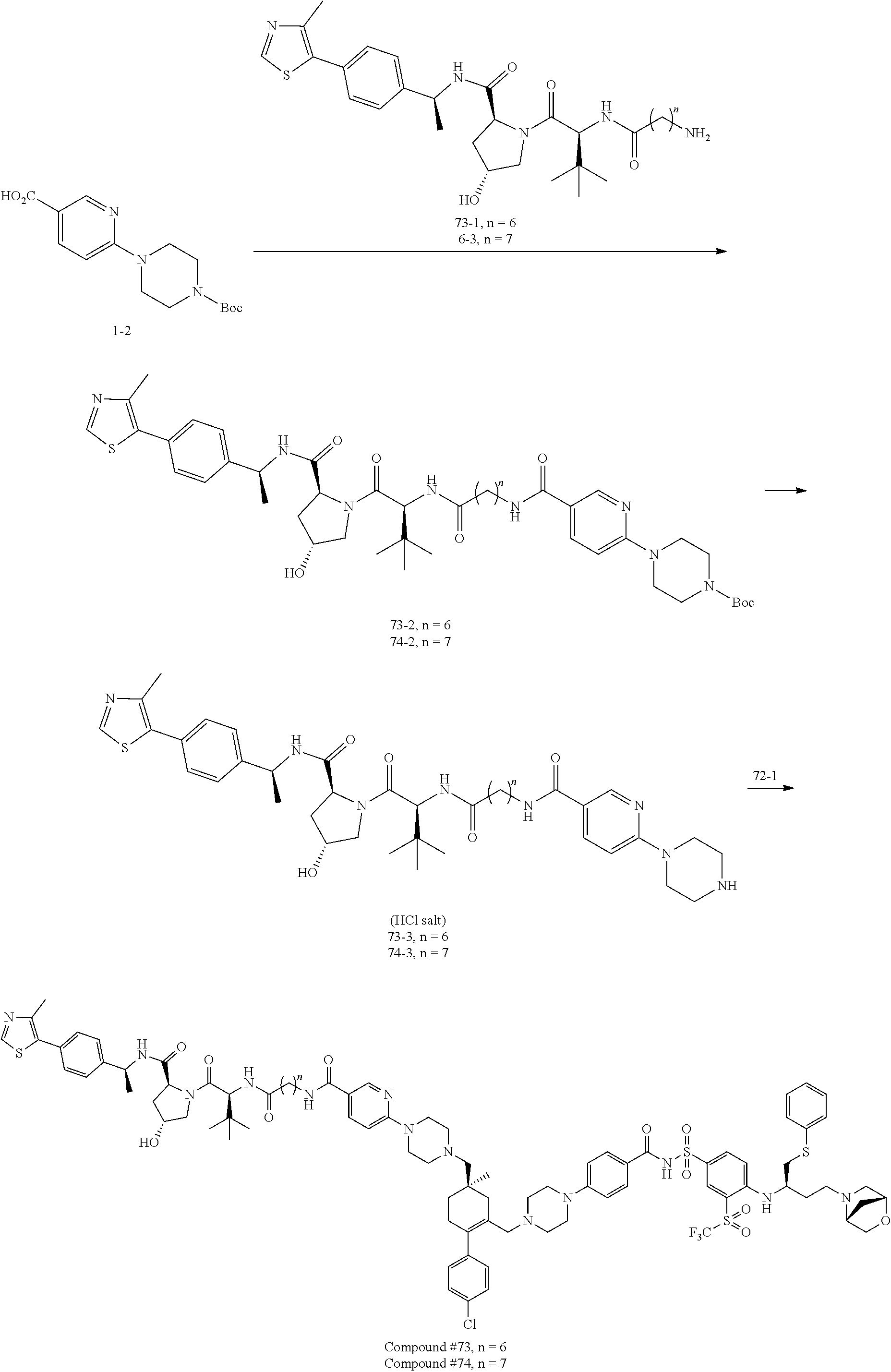

Compounds #73-74 were prepared by following steps 3-5 of General Procedure A. tert-butyl 4-(5-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)carbamoyl)pyridin-2-yl)piperazine-1-carboxylate (73-2) (92 mg, 66% yield). ¹H NMR (600 MHz, CDCl₃) δ 8.68 (s, 1H), 8.59 (d, J=2.5 Hz, 1H), 8.33 (d, J=2.3 Hz, 1H), 8.10 (dd, J=9.2, 2.3 Hz, 1H), 7.94 (dd, J=9.0, 2.5 Hz, 1H), 7.70 (dd, J=9.3, 3.6 Hz, 2H), 7.43 (d, J=7.8 Hz, 1H), 7.39 (td, J=6.6, 3.1 Hz, 4H), 7.35 (d, J=8.3 Hz, 2H), 7.34-7.22 (m, 5H), 7.03-6.96 (m, 2H), 6.81-6.74 (m, 2H), 6.63-6.55 (m, 3H), 5.07 (p, J=7.1 Hz, 1H), 4.65-4.58 (m, 2H), 4.47 (s, 1H), 4.41 (t, J=2.0 Hz, 1H), 4.05 (d, J=11.4 Hz, 1H), 3.97 (d, J=8.0 Hz, 1H), 3.94-3.90 (m, 1H), 3.65-3.53 (m, 6H), 3.46-3.39 (m, 4H), 3.30-3.20 (m, 4H), 3.10 (dd, J=13.6, 4.8 Hz, 1H), 3.05 (dd, J=13.9, 6.9 Hz, 1H), 2.91-2.81 (m, 3H), 2.73-2.59 (m, 6H), 2.51 (s, 4H), 2.45-2.19 (m, 18H), 2.08 (dd, J=13.5, 8.0 Hz, 1H), 2.05-1.98 (m, 1H), 1.92 (d, J=17.2 Hz, 1H), 1.85 (d, J=9.9 Hz, 1H), 1.77-1.53 (m, 4H), 1.46 (d, J=6.9 Hz, 3H), 1.44-1.36 (m, 1H), 1.04 (s, 9H), 0.97 (s, 3H) ppm. LC/MS (ESI) m/z 861.3; [M+H]⁺ calcd for C₄₁H₅₇N₈O₇S⁺: 861.47.

tert-butyl 4-(5-((8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamoyl)pyridin-2-yl)piperazine-1-carboxylate (74-2) (101 mg, 71% yield). ¹H NMR (600 MHz, CDCl₃) δ 8.68 (s, 1H), 8.59 (d, J=2.5 Hz, 1H), 8.33 (d, J=2.3 Hz, 1H), 8.10 (dd, J=9.2, 2.3 Hz, 1H), 7.94 (dd, J=9.0, 2.5 Hz, 1H), 7.70 (dd, J=9.3, 3.6 Hz, 2H), 7.43 (d, J=7.8 Hz, 1H), 7.39 (td, J=6.6, 3.1 Hz, 4H), 7.35 (d, J=8.3 Hz, 2H), 7.34-7.22 (m, 5H), 7.03-6.96 (m, 2H), 6.81-6.74 (m, 2H), 6.63-6.55 (m, 3H), 5.07 (p, J=7.1 Hz, 1H), 4.65-4.58 (m, 2H), 4.47 (s, 1H), 4.41 (t, J=2.0 Hz, 1H), 4.05 (d, J=11.4 Hz, 1H), 3.97 (d, J=8.0 Hz, 1H), 3.94-3.90 (m, 1H), 3.65-3.53 (m, 6H), 3.46-3.39 (m, 4H), 3.30-3.20 (m, 4H), 3.10 (dd, J=13.6, 4.8 Hz, 1H), 3.05 (dd, J=13.9, 6.9 Hz, 1H), 2.91-2.81 (m, 3H), 2.73-2.59 (m, 6H), 2.51 (s, 4H), 2.45-2.19 (m, 18H), 2.08 (dd, J=13.5, 8.0 Hz, 1H), 2.05-1.98 (m, 1H), 1.92 (d, J=17.2 Hz, 1H), 1.85 (d, J=9.9 Hz, 1H), 1.77-1.53 (m, 4H), 1.46 (d, J=6.9 Hz, 3H), 1.44-1.36 (m, 1H), 1.04 (s, 9H), 0.97 (s, 3H) ppm. LC/MS (ESI) m/z 875.6; [M+H]⁺ calcd for C₄₈H₆₇N₈O₇S⁺: 875.48.

N-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)-6-(piperazin-1-yl)nicotinamide hydrochloride (73-3) (23 mg, quantitative yield). LC/MS (ESI) m/z 761.5; [M+H]⁺ calcd for C₄₀H₅₇N₈O₅S⁺: 761.42.

N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)-6-(piperazin-1-yl)nicotinamide hydrochloride (74-3) (25 mg, quantitative yield). LC/MS (ESI) m/z 775.6; [M+H]⁺ calcd for C₄₁H₅₉N₈O₅S⁺: 775.43.

6-(4-(((R)-6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)nicotinamide (compound #73) (5.5 mg, 21% yield). ¹H NMR (600 MHz, CDCl₃) δ 8.68 (s, 1H), 8.57 (d, J=2.5 Hz, 1H), 8.33 (d, J=2.3 Hz, 1H), 8.10 (dd, J=9.3, 2.3 Hz, 1H), 7.90 (dd, J=9.0, 2.5 Hz, 1H), 7.72-7.67 (m, 2H), 7.47-7.42 (m, 1H), 7.41-7.37 (m, 4H), 7.35 (d, J=8.3 Hz, 2H), 7.34-7.21 (m, 5H), 7.02-6.97 (m, 2H), 6.80-6.75 (m, 2H), 6.61-6.55 (m, 3H), 5.07 (p, J=7.1 Hz, 1H), 4.68-4.59 (m, 2H), 4.47 (s, 1H), 4.41 (t, J=2.0 Hz, 1H), 4.07 (d, J=11.3 Hz, 1H), 3.96 (d, J=8.0 Hz, 1H), 3.95-3.89 (m, 1H), 3.63-3.55 (m, 6H), 3.46-3.34 (m, 3H), 3.25 (t, J=5.2 Hz, 4H), 3.13-3.01 (m, 2H), 2.89-2.81 (m, 3H), 2.73-2.58 (m, 6H), 2.55 (d, J=10.1 Hz, 1H), 2.52 (s, 3H), 2.43-2.20 (m, 18H), 2.12-2.07 (m, 1H), 2.04-1.98 (m, 1H), 1.92 (d, J=17.2 Hz, 1H), 1.84 (d, J=9.9 Hz, 1H), 1.75-1.53 (m, 6H), 1.46 (d, J=7.0 Hz, 3H), 1.42-1.29 (m, 2H), 1.04 (s, 9H), 0.98 (s, 3H) ppm. LC/MS (ESI) m/z 1744.9; [M+H]⁺ calcd for C₈₈H₁₁₀ClF₃N₁₃O₁₁S₄⁺: 1744.70.

6-(4-(((R)-6-((4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)nicotinamide (compound #74) (4.5 mg, 17% yield). ¹H NMR (600 MHz, CDCl₃) δ 8.68 (s, 1H), 8.55 (d, J=2.5 Hz, 1H), 8.33 (d, J=2.2 34 Hz, 1H), 8.11 (dd, J=9.3, 2.3 Hz, 1H), 7.90 (dd, J=9.0, 2.5 Hz, 1H), 7.72-7.67 (m, 2H), 7.44-7.34 (m, 7H), 7.34-7.23 (m, 5H), 7.03-6.97 (m, 2H), 6.81-6.76 (m, 2H), 6.59 (d, J=9.2 Hz, 2H), 6.53 (dd, J=11.9, 7.4 Hz, 1H), 5.08 (p, J=7.1 Hz, 1H), 4.69-4.60 (m, 2H), 4.47 (s, 1H), 4.41 (t, J=2.0 Hz, 1H), 4.05 (d, J=11.4 Hz, 1H), 3.96 (d, J=8.0 Hz, 1H), 3.94 (d, J=12.1 Hz, 1H), 3.63-3.55 (m, 6H), 3.47-3.36 (m, 3H), 3.26 (t, J=5.1 Hz, 4H), 3.13-3.02 (m, 2H), 2.88-2.80 (m, 3H), 2.73-2.60 (m, 6H), 2.58-2.49 (m, 4H), 2.40-2.18 (m, 18H), 2.12-2.04 (m, 1H), 2.04-1.98 (m, 1H), 1.93 (d, J=17.3 Hz, 1H), 1.85 (d, J=9.9 Hz, 1H), 1.77-1.52 (m, 6H), 1.47 (d, J=7.0 Hz, 3H), 1.40-1.28 (m, 4H), 1.04 (s, 9H), 0.98 (s, 3H) ppm. LC/MS (ESI) m/z 1758.9; [M+H]⁺ calcd for C₈₉H₁₁₂ClF₃N₁₃O₁₁S₄⁺: 1758.71.

### Example 39: Preparation of Compound #75

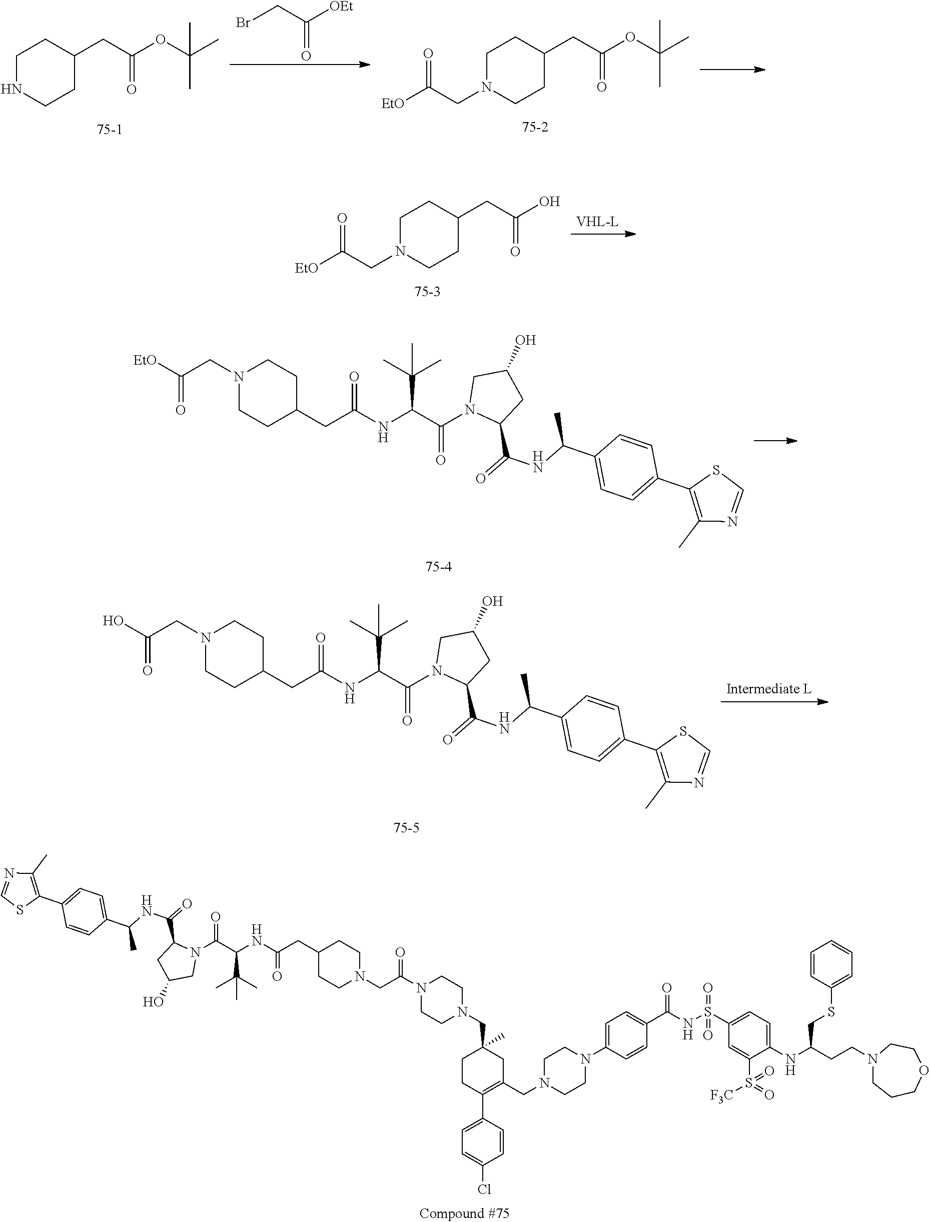

Compound #75 was prepared by following General Procedure C. tert-butyl 2-(1-(2-ethoxy-2-oxoethyl)piperidin-4-yl)acetate (75-2) (121 mg, 85% yield). ¹H NMR (600 MHz, Chloroform-d) δ 4.20 (q, J=7.1 Hz, 2H), 3.21 (s, 2H), 2.94 (dt, J=10.9, 3.0 Hz, 2H), 2.20 (td, J=11.7, 2.5 Hz, 2H), 2.16 (d, J=6.9 Hz, 2H), 1.81-1.68 (m, 3H), 1.46 (s, 9H), 1.45-1.34 (m, 2H), 1.29 (t, J=7.2 Hz, 3H) ppm. LC/MS (ESI) m/z 286.0; [M+H]⁺ calcd for C₁₅H₂₈NO₄⁺: 286.20.

2-(1-(2-ethoxy-2-oxoethyl)piperidin-4-yl)acetic acid (75-3) (40 mg, quantitative yield). LC/MS (ESI) m/z 230.0; [M+H]⁺ calcd for C₁₁H₂₀NO₄⁺: 230.14.

ethyl 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)acetate (75-4) (85 mg, 66% yield). ¹H NMR (600 MHz, CDCl₃) δ 8.69 (d, J=2.6 Hz, 1H), 7.46-7.33 (m, 5H), 6.25 (d, J=8.6 Hz, 1H), 5.10 (p, J=7.0 Hz, 1H), 4.73 (t, J=7.9 Hz, 1H), 4.56 (d, J=8.6 Hz, 1H), 4.53 (dt, J=4.5, 2.2 Hz, 1H), 4.19 (q, J=7.1 Hz, 2H), 4.13 (dt, J=11.7, 1.8 Hz, 1H), 3.61 (dd, J=11.4, 3.6 Hz, 1H), 3.20 (s, 2H), 2.97-2.89 (m, 2H), 2.58-2.46 (m, 4H), 2.21-2.06 (m, 5H), 1.83-1.74 (m, 1H), 1.68 (td, J=13.6, 2.9 Hz, 2H), 1.49 (d, J=6.9 Hz, 3H), 1.41 (qt, J=11.7, 5.1 Hz, 2H), 1.28 (t, J=7.1 Hz, 3H), 1.06 (s, 9H) ppm. LC/MS (ESI) m/z 656.5; [M+H]⁺ calcd for C₃₄H₅₀N₅O₆S⁺: 656.35.

2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)acetic acid (75-5) (69 mg, 85% yield). LC/MS (ESI) m/z 628.5; [M+H]⁺ calcd for C₃₂H₄₆N₅O₆S⁺: 628.32.

(2S,4R)-1-((S)-2-(2-(1-(2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-2-oxoethyl)piperidin-4-yl)acetamido)-3,3-dimethylbutanoyl)-4-hydroxy-N—((S)-1-(4-(4-methylthiazol-5-yl)phenyl)ethyl)pyrrolidine-2-carboxamide (compound #75) (6.7 mg, 38% yield). ¹H NMR (600 MHz, Chloroform-d) δ 8.68 (s, 1H), 8.34 (s, 1H), 8.06 (d, J=9.1 Hz, 1H), 7.82 (d, J=8.0 Hz, 2H), 7.46 (d, J=9.9 Hz, 1H), 7.39 (q, J=7.8 Hz, 6H), 7.34-7.26 (m, 4H), 7.24 (t, 1H), 7.07-6.97 (m, 2H), 6.77 (d, J=8.5 Hz, 2H), 6.58 (d, J=9.4 Hz, 1H), 5.10 (t, J=7.3 Hz, 1H), 4.75 (t, J=7.8 Hz, 1H), 4.62 (d, J=8.6 Hz, 1H), 4.51 (s, 1H), 4.13 (d, J=11.4 Hz, 1H), 3.88 (s, 1H), 3.76 (t, J=6.1 Hz, 2H), 3.75-3.66 (m, 2H), 3.64-3.15 (m, 9H), 3.15-2.96 (m, 3H), 2.96-2.80 (m, 1H), 2.79-2.02 (m, 34H), 1.95-1.76 (m, 3H), 1.76-1.55 (m, 3H), 1.55-1.24 (m, 9H), 1.06 (s, 9H), 0.95 (s, 3H) ppm. LC/MS (ESI) m/z 1681.7; [M+H]⁺ calcd for Cs₄H₁₀₉ClF₃N₁₂O₁₁S₄⁺: 1681.69.

### Example 40: Preparation of Compound #76

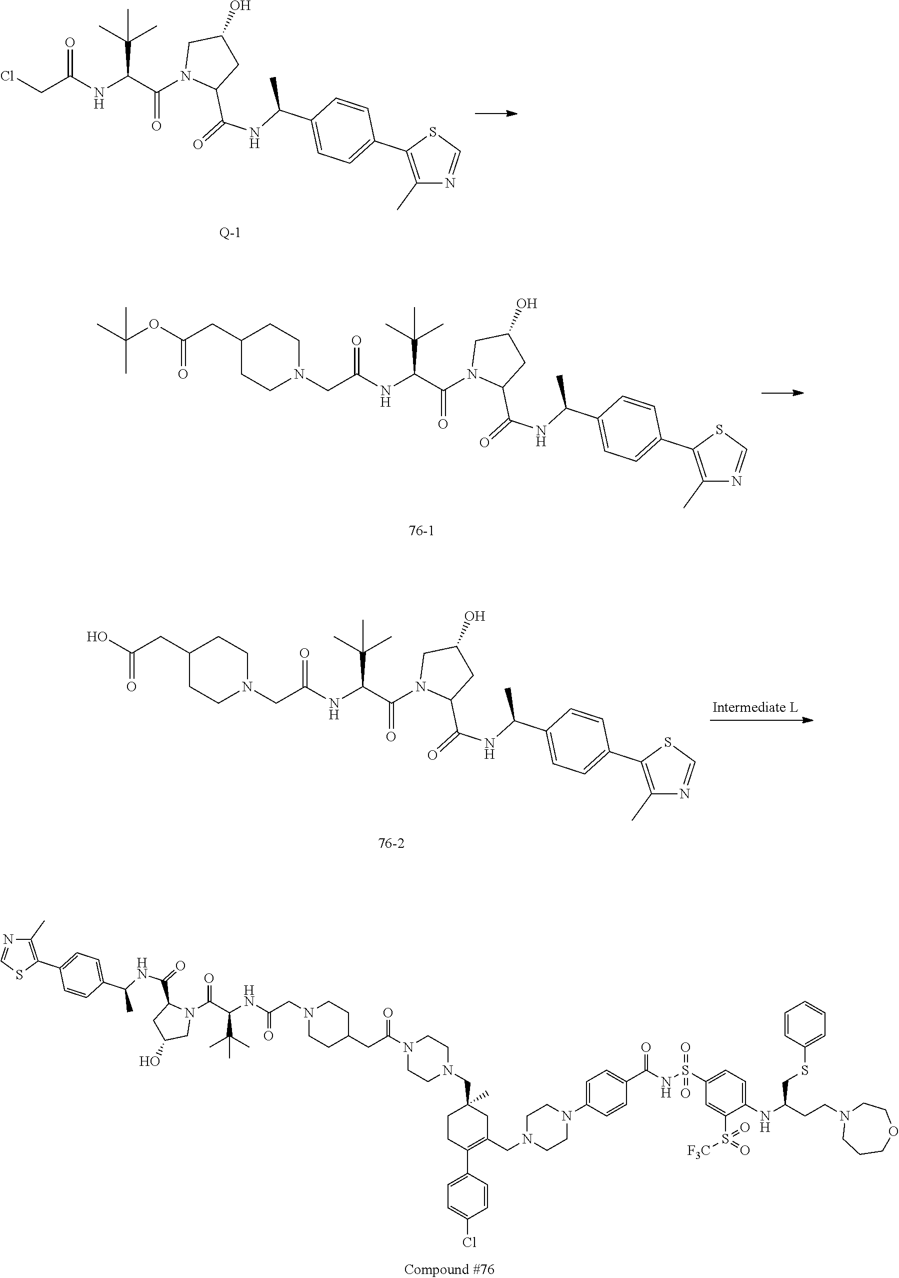

Preparation of tert-butyl 2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)acetate (76-1): A mixture of Q-1 (50 mg, 0.096 mmol), tert-butyl 2-(piperidin-4-yl)acetate (23 mg, 0.115 mmol), and DIPEA (50 μL, 0.288 mmol) in THF (3 mL) was stirred at room temperature overnight. The reaction mixture was washed with sat. aq. NH₄Cl. The organic layers were dried over Na₂SO₄, filtered, and concentrated. The residue was purified by flash column chromatography (0% to 10% of MeOH in DCM) to afford 76-1 (49 mg, 74% yield). ¹H NMR (600 MHz, CDCl₃) δ 8.69 (s, 1H), 7.94 (d, J=8.2 Hz, 1H), 7.51 (d, J=7.8 Hz, 1H), 7.43 (d, 2H), 7.39 (d, J=8.2 Hz, 2H), 5.10 (p, J=7.1 Hz, 1H), 4.79 (t, J=7.9 Hz, 1H), 4.52 (tt, J=4.1, 1.9 Hz, 1H), 4.44 (d, J=8.2 Hz, 1H), 4.23 (dq, J=11.6, 2.0 Hz, 1H), 3.60 (dd, J=11.5, 3.6 Hz, 1H), 3.00 (q, 2H), 2.85 (d, J=11.3 Hz, 2H), 2.63-2.56 (m, 1H), 2.55 (s, 3H), 2.29-2.14 (m, 4H), 2.08 (ddt, J=13.5, 8.4, 2.0 Hz, 1H), 1.84-1.67 (m, 4H), 1.50 (d, J=6.9 Hz, 3H), 1.47 (s, 9H), 1.40-1.25 (m, 2H), 1.09 (s, 9H) ppm. LC/MS (ESI) m/z 684.2; [M+H]+ calcd for C₃₆H₅₄N₅O₆S⁺: 684.38.

Preparation of 2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)acetic acid (76-2): HCl dioxane solution (4N 0.5 mL) was added to a stirring solution of 76-1 (49 mg, 0.029 mmol) in DCM (1 mL). After stirring at room temperature for 2 hours, the reaction mixture was concentrated. The residue was triturated with diethyl ether and dried under vacuum to afford 76-2 (39 mg, 87% yield), which was used in the next step without further purification. LC/MS (ESI) m/z 628.5; [M+H]⁺ calcd for C₃₂H₄₆N₅O₆S⁺: 628.32.

Preparation of (2S,4R)-1-((S)-2-(2-(4-(2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-2-oxoethyl)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #76): General Procedure H was applied to afford compound #76 (4.9 mg, 13% yield). ¹H NMR (600 MHz, Chloroform-d) δ 8.69 (s, 1H), 8.34 (d, J=2.3 Hz, 1H), 8.09 (dd, J=9.3, 2.3 Hz, 1H), 7.94 (d, J=8.4 Hz, 1H), 7.77 (d, J=8.6 Hz, 2H), 7.44-7.36 (m, 7H), 7.34-7.23 (m, 5H), 7.13 (d, J=8.5 Hz, 1H), 7.04-6.98 (m, 2H), 6.78 (d, J=8.7 Hz, 2H), 6.60 (d, J=9.4 Hz, 1H), 5.10 (p, J=7.1 Hz, 1H), 4.78 (t, J=8.0 Hz, 1H), 4.52 (d, J=8.4 Hz, 2H), 4.22 (d, J=11.5 Hz, 1H), 3.92 (d, J=10.1 Hz, 1H), 3.78 (t, J=6.1 Hz, 2H), 3.75-3.52 (m, 5H), 3.43 (d, J=5.1 Hz, 2H), 3.25 (q, J=5.0 Hz, 4H), 3.15-2.96 (m, 4H), 2.91-2.81 (m, 4H), 2.80-2.58 (m, 4H), 2.57-2.48 (m, 6H), 2.47-2.04 (m, 16H), 1.98-1.86 (m, 3H), 1.87-1.70 (m, 3H), 1.63 (dp, J=20.3, 7.3 Hz, 2H), 1.54-1.43 (m, 4H), 1.38-1.25 (m, 5H), 1.09 (s, 9H), 0.98 (s, 3H) ppm. LC/MS (ESI) m/z 1681.7; [M+H]⁺ calcd for Cs₄H₁₀₉ClF₃N₁₂O₁₁S₄⁺: 1681.69.

### Example 41: Preparation of Compound #77

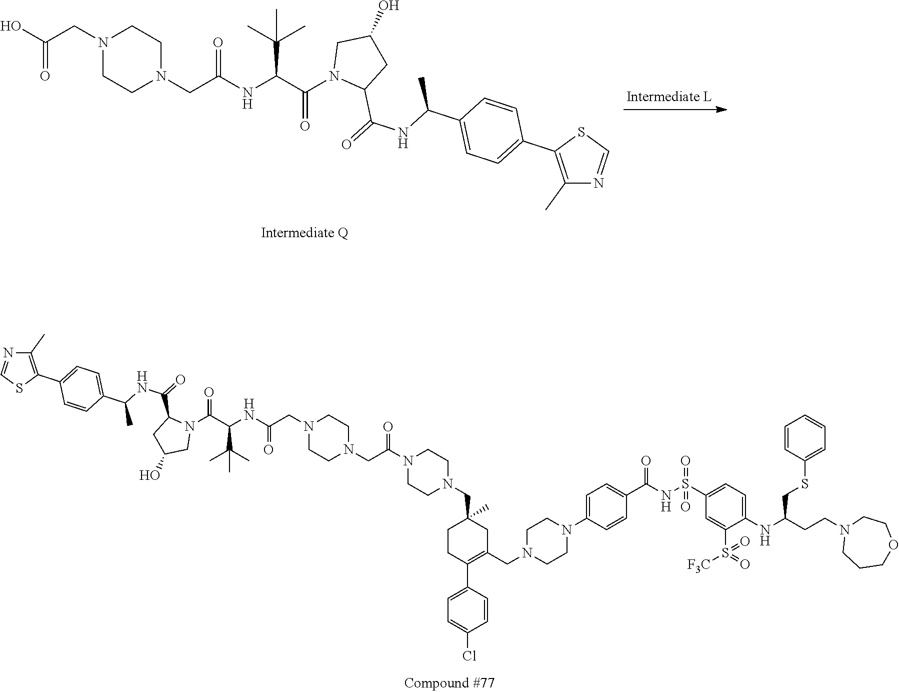 [SMILES: Cc1ncsc1-c1ccc([C@H](C)NC(=O)C2C[C@@H](O)CN2C(=O)[C@@H](NC(=O)CN2CCN(CC(=O)O)CC2)C(C)(C)C)cc1 ; "Intermediate Q" ; Cc1ncsc1-c1ccc([C@H](C)NC(=O)[C@@H]2C[C@@H](O)CN2C(=O)[C@@H](NC(=O)CN2CCN(CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1]

Compound #77 was prepared by following General Procedure H. (2S,4R)-1-((S)-2-(2-(4-(2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-2-oxoethyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #77) (16.5 mg, 36% yield). ¹H NMR (600 MHz, Chloroform-d) δ 8.70 (s, 1H), 8.34 (d, J=2.3 Hz, 1H), 8.11 (dd, J=9.1, 2.3 Hz, 1H), 7.84 (d, J=8.3 Hz, 1H), 7.76 (d, J=8.6 Hz, 2H), 7.46 (d, J=5.8 Hz, 1H), 7.45-7.36 (m, 7H), 7.35-7.23 (m, 5H), 7.13 (d, J=9.1 Hz, 1H), 7.04-6.99 (m, 2H), 6.79 (d, J=8.7 Hz, 2H), 6.62 (d, J=9.3 Hz, 1H), 5.11 (p, J=7.0 Hz, 1H), 4.79 (t, J=8.0 Hz, 1H), 4.54 (d, J 2=5.3 Hz, 2H), 4.25 (d, J=11.5 Hz, 1H), 3.93 (s, 1H), 3.77 (t, J=6.1 Hz, 2H), 3.71 (td, J=6.7, 2.7 Hz, 2H), 3.67-3.51 (m, 5H), 3.33-3.19 (m, 5H), 3.19-3.02 (m, 3H), 2.99-2.80 (m, 4H), 2.80-2.39 (m, 20H), 2.39-2.23 (m, 8H), 2.10 (dd, J=13.6, 8.1 Hz, 2H), 1.96-1.83 (m, 3H), 1.78-1.63 (m, 2H), 1.50 (d, J=7.0 Hz, 3H), 1.47-1.39 (m, 1H), 1.33-1.23 (m, 3H), 1.08 (s, 9H), 0.97 (s, 3H) ppm. LC/MS (ESI) m/z 1682.6; [M+H]⁺ calcd for C₈₃H₁₀₈ClF₃N₁₃O₁₁S₄⁺: 1682.68.

### Example 42: Preparation of Compounds #78-79

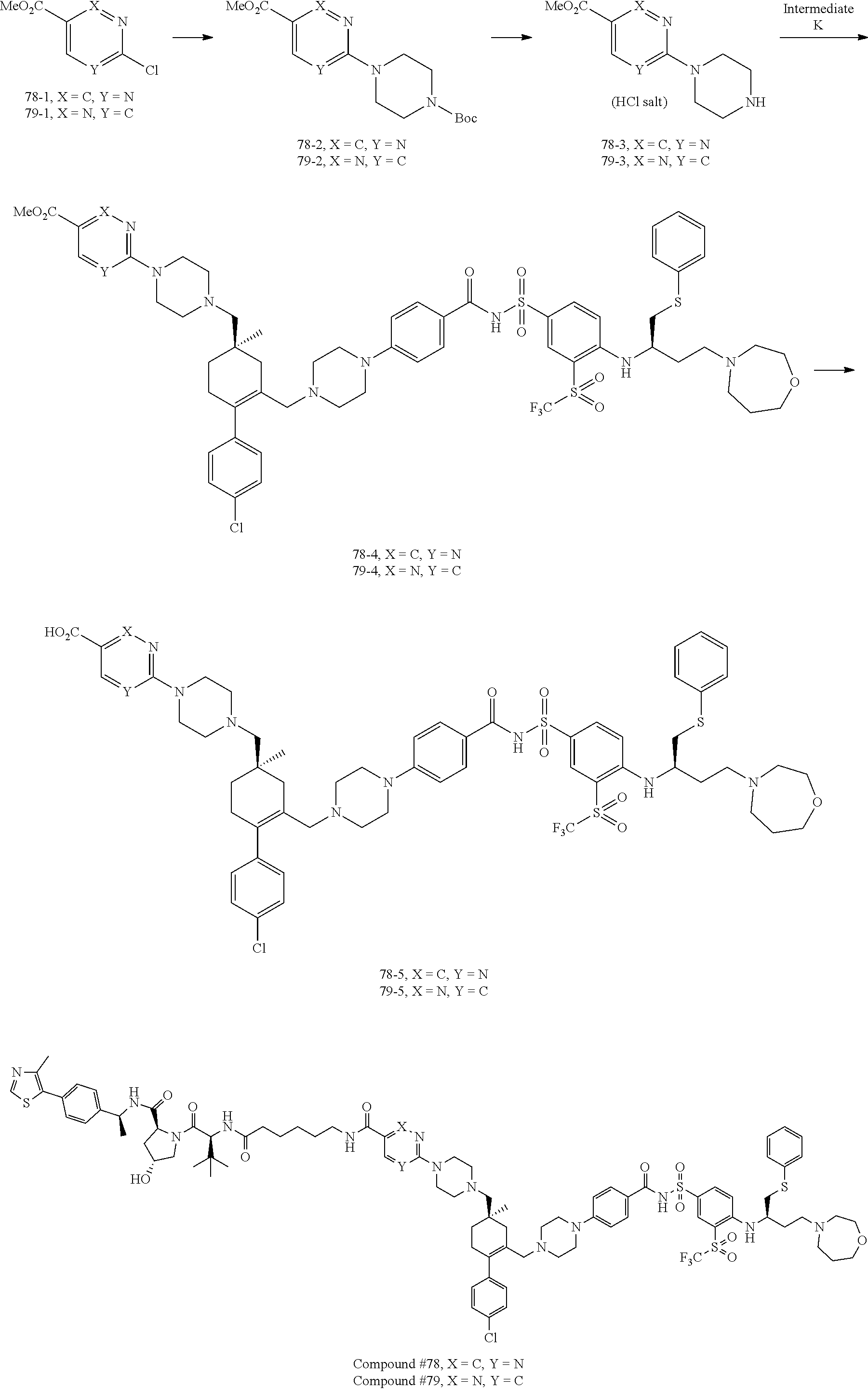

Preparation of methyl 2-(4-(tert-butoxycarbonyl)piperazin-1-yl)pyrimidine-5-carboxylate (78-2) and methyl 6-(4-(tert-butoxycarbonyl)piperazin-1-yl)pyridazine-3-carboxylate (79-2): A mixture of 78-1 or 79-1 (1.0 equiv.), 1-Boc-piperazine (1.05-1.2 equiv.) and DIPEA (3.0 equiv.) in DMF was stirred at 90° C. overnight. The reaction mixture was cooled to room temperature and diluted with water, then extracted with EtOAc. The combined organic layers were washed with water and sat. aq. NH₄Cl, then dried over Na₂SO₄, filtered, and concentrated. The residue was purified by flash column chromatography to afford 78-2 and 79-2 respectively. 78-2 (460 mg, 89% yield): ¹H NMR (600 MHz, Chloroform-d) δ 8.87 (s, 2H), 7.29 (s, 1H), 3.94 (t, J=5.3 Hz, 4H), 3.91 (s, 3H), 3.53 (t, J=5.3 Hz, 4H), 1.51 (s, 9H) ppm. LC/MS (ESI) m/z 323.3; [M+H]⁺ calcd for C₁₅H₂₃N₄O₄+: 323.17. 79-2 (420 mg, 81% yield): ¹H NMR (600 MHz, CDCl₃) δ 7.94 (d, J=9.5 Hz, 1H), 6.89 (d, J=9.5 Hz, 1H), 4.02 (s, 3H), 3.82 (s, 4H), 3.62 (dd, J=6.6, 4.0 Hz, 4H), 1.51 (s, 9H) ppm. LC/MS (ESI) m/z 323.0; [M+H]⁺ calcd for C₁₅H₂₃N₄O₄⁺: 323.17.

Preparation of methyl 2-(piperazin-1-yl)pyrimidine-5-carboxylate hydrochloride (78-3) and methyl 6-(piperazin-1-yl)pyridazine-3-carboxylate hydrochloride (79-3): To a solution of 78-2 or 79-2 in DCM was added HCl dioxane solution (4N), then stirred at room temperature for 2 hours. The reaction mixture was concentrated to afford crude 78-3 and 79-3, respectively, as HCl salts, which were used directly in the next step. 78-3 (42 mg, quantitative yield): LC/MS (ESI) m/z 223.3; [M+H]⁺ calcd for C₁₀H₁₅N₄O₂⁺: 223.12. 79-3 (51 mg, quantitative yield): LC/MS (ESI) m/z 223.1; [M+H]⁺ calcd for C₁₀H₁₅N₄O₂⁺: 223.12.

Preparation of methyl 2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyrimidine-5-carboxylate (78-4) and methyl 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyridazine-3-carboxylate (79-4): A suspension of 78-3 or 79-3 (1.2 equiv., HCl salt), TEA (10.0 equiv.), Intermediate K (1.0 equiv.), and NaBH(OAc)₃ (2.0 equiv.) in DCM was stirred at room temperature overnight. The reaction mixture was washed with sat. aq. NH₄Cl, dried over Na₂SO₄, filtered, and concentrated. The residue was purified by flash column chromatography to afford 78-4 and 79-4, respectively. 78-4 (9 mg, 31% yield). ¹H NMR (600 MHz, Chloroform-d) δ 9.15 (dd, J=2.1, 0.9 Hz, 1H), 8.37 (dd, J=8.1, 2.1 Hz, 1H), 8.33 (d, J=2.2 Hz, 1H), 8.04 (dd, J=9.3, 2.3 Hz, 1H), 7.72 (d, J=8.9 Hz, 2H), 7.69 (d, J=8.0, 0.8 Hz, 1H), 7.38-7.33 (m, 2H), 7.31-7.20 (m, 5H), 7.07 (d, J=8.5 Hz, 1H), 6.98 (d, 2H), 6.75 (d, 2H), 3.98 (s, 3H), 3.94-3.85 (m, 1H), 3.85-3.68 (m, 6H), 3.57-3.45 (m, 2H), 3.24 (t, J=5.1 Hz, 4H), 3.05 (dddd, 2H), 2.91-2.75 (m, 4H), 2.75-2.60 (m, 5H), 2.56 (h, J=7.0 Hz, 2H), 2.39 (t, J=5.1 Hz, 4H), 2.34-2.13 (m, 5H), 2.13-2.06 (m, 1H), 1.99-1.86 (m, 3H), 1.76 (h, J=13.8, 7.0 Hz, 1H), 1.66-1.53 (m, 1H), 1.51-1.40 (m, 1H), 1.35-1.22 (m, 2H), 0.94 (s, 3H) ppm. LC/MS (ESI) m/z 1208.5; [M+H]⁺ calcd for C₅₈H₇₀ClF₃N₉O₈S₃⁺: 1208.41. 79-4 (10 mg, 33% yield): ¹H NMR (600 MHz, Chloroform-d) δ 8.32 (d, J=2.2 Hz, 1H), 8.03 (dd, J=9.2, 2.2 Hz, 1H), 7.85 (d, J=9.6 Hz, 1H), 7.73 (d, J=8.7 Hz, 2H), 7.39-7.32 (m, 2H), 7.31-7.20 (m, 5H), 7.05 (d, J=8.5 Hz, 1H), 7.02-6.96 (m, 2H), 6.82 (d, J=9.6 Hz, 1H), 6.75 (d, J=8.9 Hz, 2H), 6.55 (d, J=9.3 Hz, 1H), 3.98 (s, 3H), 3.92-3.83 (m, 1H), 3.81-3.67 (m, 7H), 3.27 (t, J=5.1 Hz, 4H), 3.05 (dddd, 2H), 2.95-2.87 (m, 2H), 2.87-2.75 (m, 2H), 2.75-2.60 (m, 7H), 2.44 (s, 4H), 2.36-2.15 (m, 4H), 2.15-2.04 (m, 1H), 2.00-1.87 (m, 3H), 1.76 (h, J=13.8, 7.0 Hz, 1H), 1.64 (h, 1H), 1.53-1.42 (m, 1H), 1.35-1.20 (m, 2H), 0.97 (s, 3H) ppm. LC/MS (ESI) m/z 1208.4; [M+H]⁺ calcd for C₅₈H₇₀ClF₃N₉O₈S₃⁺: 1208.41.

Preparation of 2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyrimidine-5-carboxylic acid (78-5) and 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyridazine-3-carboxylic acid (79-5): To a solution of 78-4 or 79-4 (1.0 equiv.) in MeOH/THF was added a solution of LiOH·H₂O (5 equiv.) in water, then stirred at 40° C. overnight. The reaction mixture was concentrated to remove the organic solvents, then adjusted to pH=5˜6 with 10% aq. citric acid and extracted with EtOAc. The combined organic layers were washed with brine, dried over Na₂SO₄, filtered, and concentrated to afford crude 78-5 and 79-5, respectively, which was used directly in the next step. 78-5 (11 mg, 96% yield): LC/MS (ESI) m/z 1194.5; [M+H]⁺ calcd for C₅₇H₆₈ClF₃N₉O₈S₃⁺: 1194.40. 79-5 (9.3 mg, 95% yield): LC/MS (ESI) m/z 1194.3; [M+H]⁺ calcd for C₅₇H₆₈ClF₃N₉O₈S₃⁺: 1194.40.

Preparation of 2-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)pyrimidine-5-carboxamide (compound #78) and 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)pyridazine-3-carboxamide (compound #79): A solution of 78-5 or 79-5 (1.0 equiv.), acid (1.0 equiv., from step 4), HATU (1.5 equiv.), and TEA (5.0 equiv.) in DCM. The reaction mixture was stirred at room temperature overnight, then washed with sat. aq. NH₄Cl. The organic layer was dried over Na₂SO₄, filtered, and concentrated. The residue was purified by preparative TLC (DCM/MeOH) to afford compound #78 and compound #79, respectively. Compound #78 (3.2 mg, 47% yield). ¹H NMR (600 MHz, CDCl₃) δ 8.76 (s, 2H), 8.68 (s, 1H), 8.33 (d, J=2.3 Hz, 1H), 8.10 (dd, J=9.3, 2.3 Hz, 1H), 7.70 (d, J=8.7 Hz, 2H), 7.44-7.33 (m, 7H), 7.33-7.22 (m, 4H), 7.04 (t, J=5.8 Hz, 1H), 7.00 (d, J=8.3 Hz, 2H), 6.77 (d, J=8.7 Hz, 2H), 6.60 (dd, J=9.3, 6.8 Hz, 2H), 5.08 (p, J=7.1 Hz, 1H), 4.65-4.58 (m, 2H), 4.48 (s, 1H), 4.05 (d, J=11.4 Hz, 1H), 3.90 (s, 1H), 3.84 (s, 4H), 3.76 (t, J=6.1 Hz, 2H), 3.73-3.65 (m, 2H), 3.61 (dd, J=11.4, 3.4 Hz, 1H), 3.45-3.39 (m, 2H), 3.25 (t, J=5.2 Hz, 4H), 3.10 (dd, J=13.9, 5.2 Hz, 1H), 3.05 (dd, J=13.9, 6.9 Hz, 1H), 2.85 (s, 2H), 2.81-2.55 (m, 9H), 2.51 (s, 3H), 2.44-2.20 (m, 14H), 2.14-2.04 (m, 2H), 1.97-1.85 (m, 3H), 1.81-1.54 (m, 8H), 1.51-1.34 (m, 5H), 1.04 (s, 9H), 0.98 (s, 3H) ppm. LC/MS (ESI) m/z 1733.7; [M+H]⁺ calcd for C₈₆H₁₀₉ClF₃N14O₁₁S₄⁺: 1733.69. Compound #79 (3.6 mg, 43% yield). ¹H NMR (600 MHz, CDCl₃) δ 8.68 (s, 1H), 8.33 (d, J=2.4 Hz, 1H), 8.10 (dd, J=9.2, 2.3 Hz, 1H), 8.01 (t, J=6.0 Hz, 1H), 7.94 (d, J=9.5 Hz, 1H), 7.71 (d, J=8.7 Hz, 2H), 7.48 (d, J=7.8 Hz, 1H), 7.43-7.35 (m, 6H), 7.28 (td, J=15.2, 7.4 Hz, 5H), 7.00 (d, J=8.3 Hz, 2H), 6.93 (d, J=9.6 Hz, 1H), 6.78 (d, J=8.7 Hz, 2H), 6.61 (d, J=9.4 Hz, 1H), 6.56 (d, J=9.2 Hz, 1H), 5.08 (p, J=7.0 Hz, 1H), 4.69 (t, J=8.1 Hz, 1H), 4.60 (d, J=9.2 Hz, 1H), 4.48 (s, 1H), 4.05 (d, J=11.5 Hz, 1H), 3.90 (s, 1H), 3.79-3.63 (m, 8H), 3.59 (dd, J=11.4, 3.4 Hz, 1H), 3.51-3.41 (m, 3H), 3.26 (t, J=5.0 Hz, 4H), 3.10 (dd, J=13.9, 5.2 Hz, 1H), 3.05 (dd, J=13.9, 6.9 Hz, 1H), 2.85 (s, 2H), 2.78-2.54 (m, 9H), 2.52 (s, 3H), 2.44-2.35 (m, 5H), 2.34-2.20 (m, 7H), 2.15-2.03 (m, 3H), 2.01 (d, J=6.4 Hz, 1H), 1.96 (d, J=17.3 Hz, 1H), 1.91-1.85 (m, 1H), 1.70-1.56 (m, 8H), 1.48 (d, J=7.0 Hz, 3H), 1.40 (p, J=7.8 Hz, 2H), 1.03 (s, 9H), 0.99 (s, 3H) ppm. LC/MS (ESI) m/z 1733.6; [M+H]⁺ calcd for C₈₆H₁₀₉ClF₃N₁₄O₁₁S₄⁺: 1733.69.

### Example 43: Preparation of Compound #80

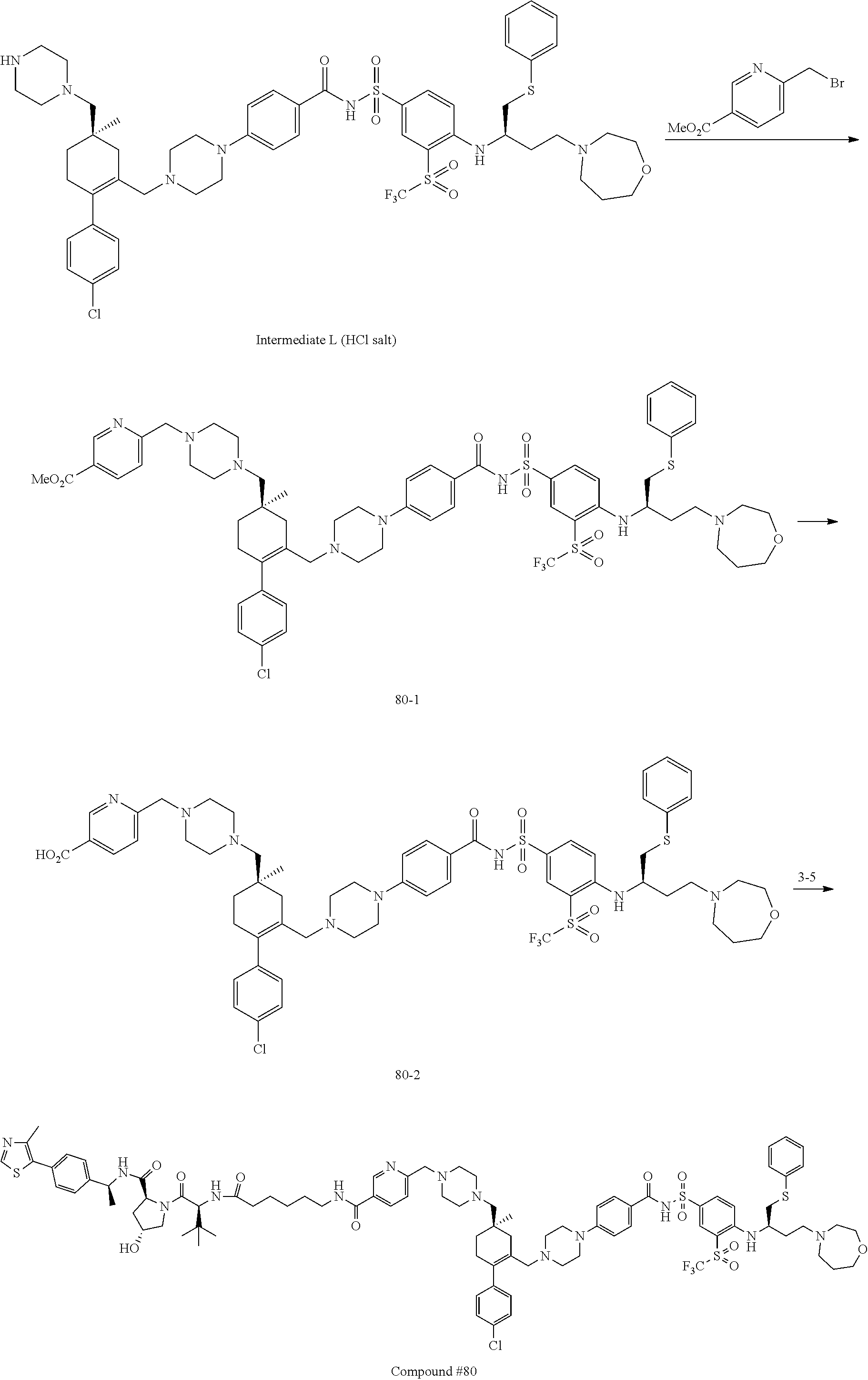

Preparation of methyl 6-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)methyl)nicotinate (80-1): A solution of methyl 6-(bromomethyl)nicotinate (5.0 mg, 0.022 mmol), Intermediate L (20 mg, 0.018 mmol, HCl salt), and DIPEA (30 μL, 0.38 mmol) in DMF (0.3 mL) was stirred at room temperature overnight. The reaction mixture was diluted with water, then extracted with EtOAc. The combined organic layers were washed with water and sat. aq. NH₄Cl, dried over Na₂SO₄, filtered, and concentrated. The residue was purified by flash column chromatography (0% to 10% of MeOH in DCM) to afford 80-1 (15 mg, 69% yield). ¹H NMR (600 MHz, CDCl₃) δ 9.15 (d, J=2.2 Hz, 1H), 8.36 (d, J=2.2 Hz, 1H), 8.25 (dt, J=8.1, 3.0 Hz, 1H), 8.04 (dd, J=9.2, 2.3 Hz, 1H), 7.77 (d, J=8.6 Hz, 2H), 7.56 (d, J=8.0 Hz, 1H), 7.42-7.36 (m, 2H), 7.34-7.22 (m, 5H), 7.07-6.97 (m, 3H), 6.77 (d, J=8.7 Hz, 2H), 6.54 (d, J=9.4 Hz, 1H), 3.96 (d, J=2.9 Hz, 3H), 3.88 (d, J=8.7 Hz, 1H), 3.82-3.72 (m, 6H), 3.27 (dt, J=18.9, 5.2 Hz, 4H), 3.11 (dd, J=13.9, 4.9 Hz, 1H), 3.04 (dd, J=13.9, 6.9 Hz, 1H), 2.93-2.78 (m, 4H), 2.78-2.57 (m, 12H), 2.40 (d, J=6.4 Hz, 4H), 2.38-2.09 (m, 6H), 2.00-1.92 (m, 3H), 1.82-1.72 (m, 1H), 1.63 (dt, J=13.5, 7.2 Hz, 2H), 1.46 (dt, J=12.3, 5.7 Hz, 1H), 0.97 (d, J=3.9 Hz, 3H) ppm. LC/MS (ESI) m/z 1221.6; [M+H]⁺ calcd for C₆₀H₇₃ClF₃N₈O₈S₃⁺: 1221.43.

Preparation of 6-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)methyl)nicotinic acid (80-2): A solution of 80-1 (10 mg, 0.0082 mmol) and LiOH·H₂O (3.36 mg, 0.08 mmol) in THF/MeOH/H₂O (0.1 mL/0.1 mL/0.1 mL) was stirred at room temperature for 2 hours. The reaction mixture was concentrated under vacuum to remove organic solvents, then adjusted to pH=4-5 and extracted with EtOAc. The combined organic layers were washed with brine, dried over Na₂SO₄, filtered, and concentrated to afford crude 80-2 (6.6 mg, 67% yield), which was used directly in the next step. LC/MS (ESI) m/z 1207.5; [M+H]⁺ calcd for C₅₉H₇₁ClF₃N₈O₈S₃⁺: 1207.42.

Preparation of 6-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)methyl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)nicotinamide (compound #80): 3-5 (3.7 mg, 0.0066 mmol) was added to a mixture of 80-2 (6.6 mg, 0.0055 mmol), EDC (4.5 mg, 0.022 mmol), and DMAP (3 mg, 0.024 mmol) in DCM (1 mL), then the resulting mixture was stirred at room temperature overnight. The reaction mixture was washed with water and sat. aq. NH₄Cl, dried over Na₂SO₄, filtered, and concentrated. The residue was purified by preparative TLC (DCM/MeOH=10/1) to afford compound #80 (4.1 mg, 43% yield). ¹H NMR (600 MHz, Chloroform-d) δ 8.93 (d, J=2.2 Hz, 1H), 8.67 (s, 1H), 8.31 (d, J=2.3 Hz, 1H), 8.15 (d, J=7.6 Hz, 1H), 8.10-8.03 (m, 2H), 7.72 (d, J=8.7 Hz, 3H), 7.64 (d, J=7.7 Hz, 1H), 7.54-7.43 (m, 1H), 7.42-7.32 (m, 6H), 7.28 (s, 4H), 6.99 (d, J=8.0 Hz, 2H), 6.77-6.66 (m, 3H), 6.61 (d, J=9.2 Hz, 1H), 5.07 (p, J=7.0 Hz, 1H), 4.66-4.55 (m, 2H), 4.49 (s, 1H), 3.99 (d, J=11.3 Hz, 1H), 3.89 (s, 1H), 3.75 (t, J=6.1 Hz, 2H), 3.72-3.56 (m, 5H), 3.50-3.42 (m, 2H), 3.40 (p, J=1.6 Hz, 2H), 3.29-3.16 (m, 5H), 3.10 (dd, J=13.9, 5.1 Hz, 1H), 3.04 (dd, J=13.9, 6.9 Hz, 1H), 2.74-2.52 (m, 8H), 2.50 (s, 3H), 2.38-2.00 (m, 20H), 1.85 (t, J=6.4 Hz, 2H), 1.74-1.54 (m, 6H), 1.47 (d, J=7.0 Hz, 3H), 1.44-1.37 (m, 2H), 1.01 (s, 9H), 0.98-0.76 (m, 4H) ppm. LC/MS (ESI) m/z 1746.8; [M+H]⁺ calcd for C₈₈H₁₁₂ClF₃N₁₃O₁₁S₄⁺: 1746.71.

### Example 44: Preparation of Compound #81

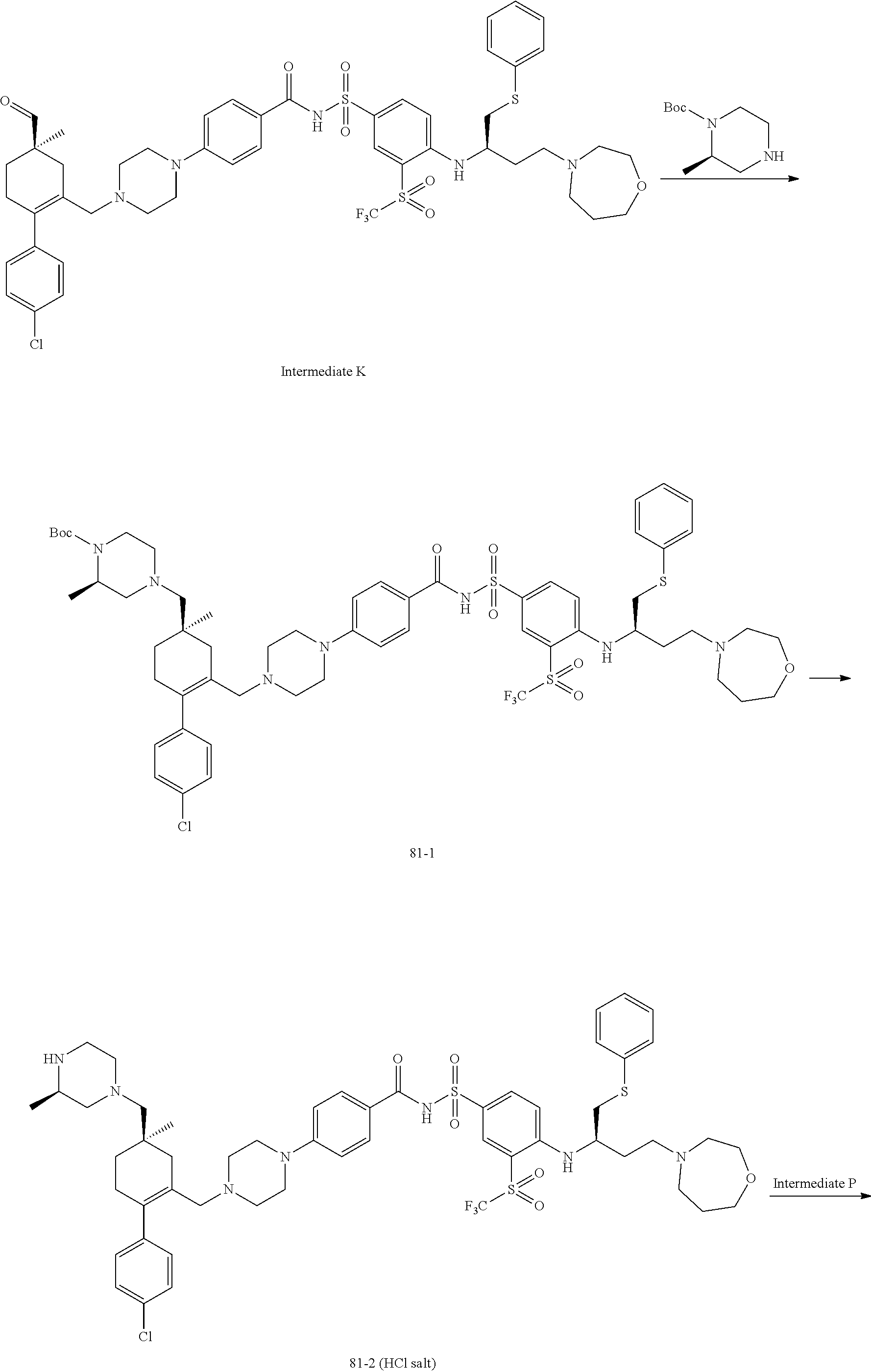

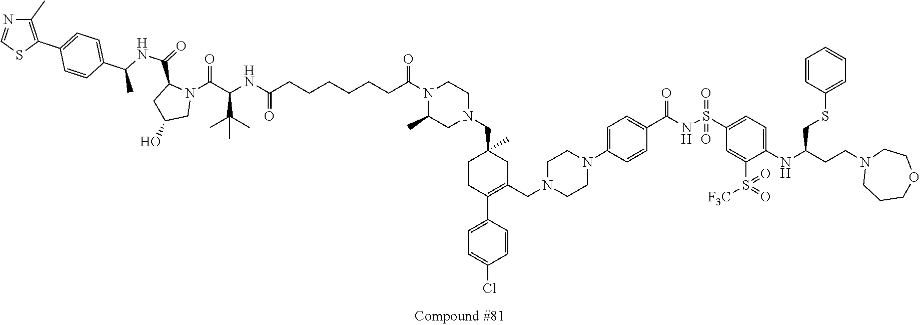 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)C[C@H]2C)C(C)(C)C)cc1]

Compound #81 was prepared by following General Procedure G. tert-butyl (R)-4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2-methylpiperazine-1-carboxylate (81-1) (10 mg, 56% yield). ¹H NMR (600 MHz, Chloroform-d) δ 8.36 (q, J=2.0 Hz, 1H), 8.13-8.06 (m, 1H), 7.72 (dd, J=9.9, 4.8 Hz, 2H), 7.41-7.37 (m, 2H), 7.36-7.24 (m, 4H), 7.16 (s, 1H), 7.02-6.99 (m, 2H), 6.80 (dd, J=8.9, 2.0 Hz, 2H), 6.61 (d, J=9.2 Hz, 1H), 4.16 (s, 1H), 3.93 (d, J=12.8 Hz, 1H), 3.81-3.67 (m, 5H), 3.28 (t, J=5.1 Hz, 4H), 3.16-2.99 (m, 3H), 2.86-2.57 (m, 12H), 2.48-2.32 (m, 5H), 2.32-2.16 (m, 5H), 2.01-1.87 (m, 4H), 1.82-1.71 (m, 1H), 1.69-1.57 (m, 1H), 1.48 (d, J=6.2 Hz, 12H), 0.98 (s, 3H) ppm. LC/MS (ESI) m/z 1186.3; [M+H]⁺ calcd for C₅₈H₇₆ClF₃N₇O₈S₃⁺: 1186.46.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-(((R)-3-methylpiperazin-1-yl)methyl)-3,4,5,6-tetrahydro-[1,1′-biphenyl]-2-yl)methyl)piperazin-1-yl)benzamide hydrochloride (81-2) (11 mg, quantitative yield). LC/MS (ESI) m/z 1086.3; [M+H]⁺ calcd for C₅₃H₆₈ClF₃N₇O₆S₃⁺: 1086.40.

(2S,4R)-1-((S)-2-(8-((R)-4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2-methylpiperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #81) (4.5 mg, 32% yield in two steps). ¹H NMR (600 MHz, CDCl₃) δ 8.70 (s, 1H), 8.35 (d, J=2.2 Hz, 1H), 8.10 (d, J=8.6 Hz, 1H), 7.73 (d, J=8.5 Hz, 2H), 7.51 (t, J=10.0 Hz, 1H), 7.44-7.37 (m, 7H), 7.35-7.23 (m, 5H), 7.16 (d, J=9.0 Hz, 1H), 7.04-6.98 (m, 2H), 6.78 (d, J=8.7 Hz, 2H), 6.62 (d, J=9.3 Hz, 1H), 6.31 (s, 1H), 5.12 (p, J=7.0 Hz, 1H), 4.77 (t, J=7.9 Hz, 1H), 4.70-4.60 (m, 1H), 4.52 (s, 1H), 4.33 (d, J=13.3 Hz, 0.5H), 4.15 (d, J=11.5 Hz, 1H), 3.93 (s, 1.5H), 3.78 (t, J=6.1 Hz, 2H), 3.75-3.66 (m, 2H), 3.60 (d, J=11.3 Hz, 1H), 3.54-3.36 (m, 1H), 3.25 (s, 4H), 3.15-3.02 (m, 2H), 2.81 (q, J=60.6 Hz, 8H), 2.57-2.47 (m, 4H), 2.47-2.05 (m, 20H), 1.97 (d, J=17.4 Hz, 1H), 1.91 (s, 2H), 1.76 (d, J=10.8 Hz, 1H), 1.70-1.53 (m, 7H), 1.53-1.39 (m, 5H), 1.38-1.28 (m, 5H), 1.07 (s, 9H), 0.99 (d, J=13.7 Hz, 3H) ppm. LC/MS (ESI) m/z 1668.8; [M+H]⁺ calcd for C₈₄H₁₁₀ClF₃N₁₁O₁₁S₄⁺: 1668.69.

### Example 45: Preparation of Compound #82

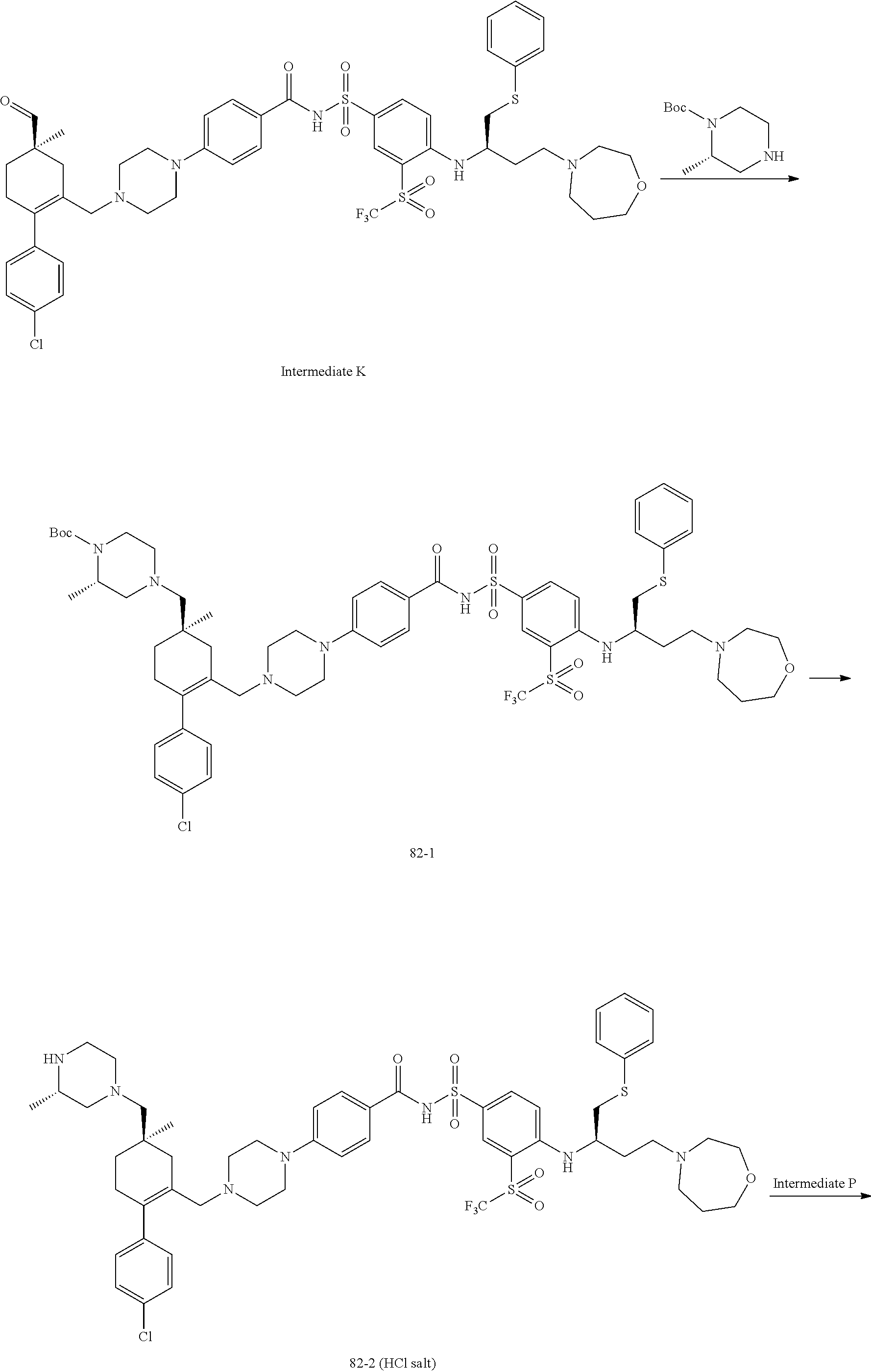

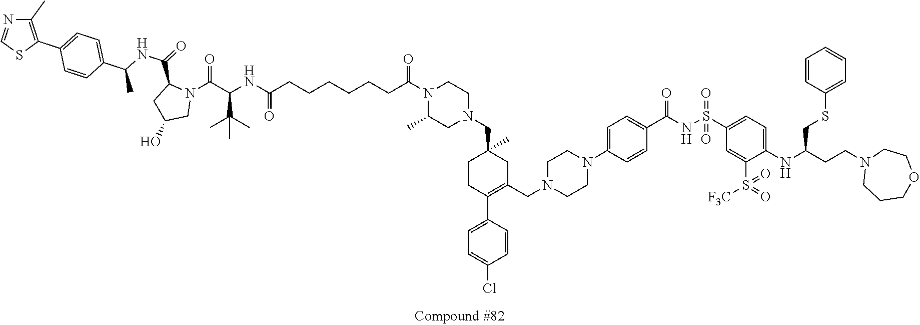 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)C[C@@H]2C)C(C)(C)C)cc1]

Compound #82 was prepared by following General Procedure G. tert-butyl (S)-4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2-methylpiperazine-1-carboxylate (82-1) (10 mg, 56% yield). ¹H NMR (600 MHz, Chloroform-d) δ 8.36 (t, J=2.6 Hz, 1H), 8.07 (t, J=7.7 Hz, 1H), 7.76-7.68 (m, 2H), 7.43-7.36 (m, 2H), 7.36-7.23 (m, 4H), 7.18-7.09 (m, 1H), 7.03-6.99 (m, 2H), 6.79 (dd, J=9.2, 2.4 Hz, 2H), 6.60 (dd, J=9.4, 3.5 Hz, 1H), 4.16 (d, J=8.3 Hz, 1H), 3.93 (d, J=9.2 Hz, 1H), 3.82-3.69 (m, 5H), 3.28 (t, J=5.0 Hz, 4H), 3.16-3.00 (m, 3H), 2.89-2.62 (m, 10H), 2.60 (d, J=11.1 Hz, 1H), 2.48 (dd, J=11.2, 3.7 Hz, 1H), 2.40 (d, J=5.5 Hz, 4H), 2.32-2.08 (m, 8H), 1.96-1.89 (m, 2H), 1.83-1.72 (m, 1H), 1.64 (dt, J=14.6, 7.1 Hz, 1H), 1.51-1.44 (m, 12H), 0.97 (d, J=1.5 Hz, 3H) ppm. LC/MS (ESI) m/z 1186.6; [M+H]⁺ calcd for C₅₈H₇₆ClF₃N₇O₈S₃⁺: 1186.46.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-(((S)-3-methylpiperazin-1-yl)methyl)-3,4,5,6-tetrahydro-[1,1′-biphenyl]-2-yl)methyl)piperazin-1-yl)benzamide hydrochloride (82-2) (10 mg, quantitative yield). LC/MS (ESI) m/z 1086.6; [M+H]⁺ calcd for C₅₃H₆₈ClF₃N₇O₆S₃⁺: 1086.40.

(2S,4R)-1-((S)-2-(8-((S)-4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2-methylpiperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #82) (2.6 mg, 18% yield in two steps). ¹H NMR (600 MHz, CDCl₃) δ 8.70 (s, 1H), 8.35 (s, 1H), 8.11 (dd, J=9.1, 2.3 Hz, 1H), 7.72 (s, 2H), 7.50 (s, 1H), 7.44-7.37 (m, 6H), 7.35-7.24 (m, 5H), 7.17 (d, J=8.6 Hz, 1H), 7.03-6.97 (m, 2H), 6.78 (d, J=8.7 Hz, 2H), 6.62 (d, J=9.4 Hz, 1H), 6.34-6.23 (m, 1H), 5.12 (p, J=7.1 Hz, 1H), 4.77 (t, J=8.0 Hz, 1H), 4.70-4.58 (m, 2H), 4.52 (s, 1H), 4.32 (d, J=13.2 Hz, 0.5H), 4.17 (d, J=10.6 Hz, 1H), 3.94 (s, 1.5H), 3.78 (t, J=6.1 Hz, 2H), 3.75-3.64 (m, 2H), 3.60 (d, J=10.9 Hz, 1H), 3.50-3.37 (m, 1H), 3.25 (s, 4H), 3.16-3.03 (m, 2H), 3.00-2.57 (m, 8H), 2.53 (s, 4H), 2.46-2.06 (m, 20H), 1.90 (s, 3H), 1.74 (d, J=8.5 Hz, 1H), 1.69-1.52 (m, 7H), 1.52-1.39 (m, 5H), 1.37-1.24 (m, 5H), 1.07 (s, 9H), 0.98 (d, J=13.9 Hz, 3H) ppm. LC/MS (ESI) m/z 1668.6; [M+H]⁺ calcd for C₈₄H₁₁₀ClF₃N₁₁O₁₁S₄⁺: 1668.69.

### Example 46: Preparation of Compound #83

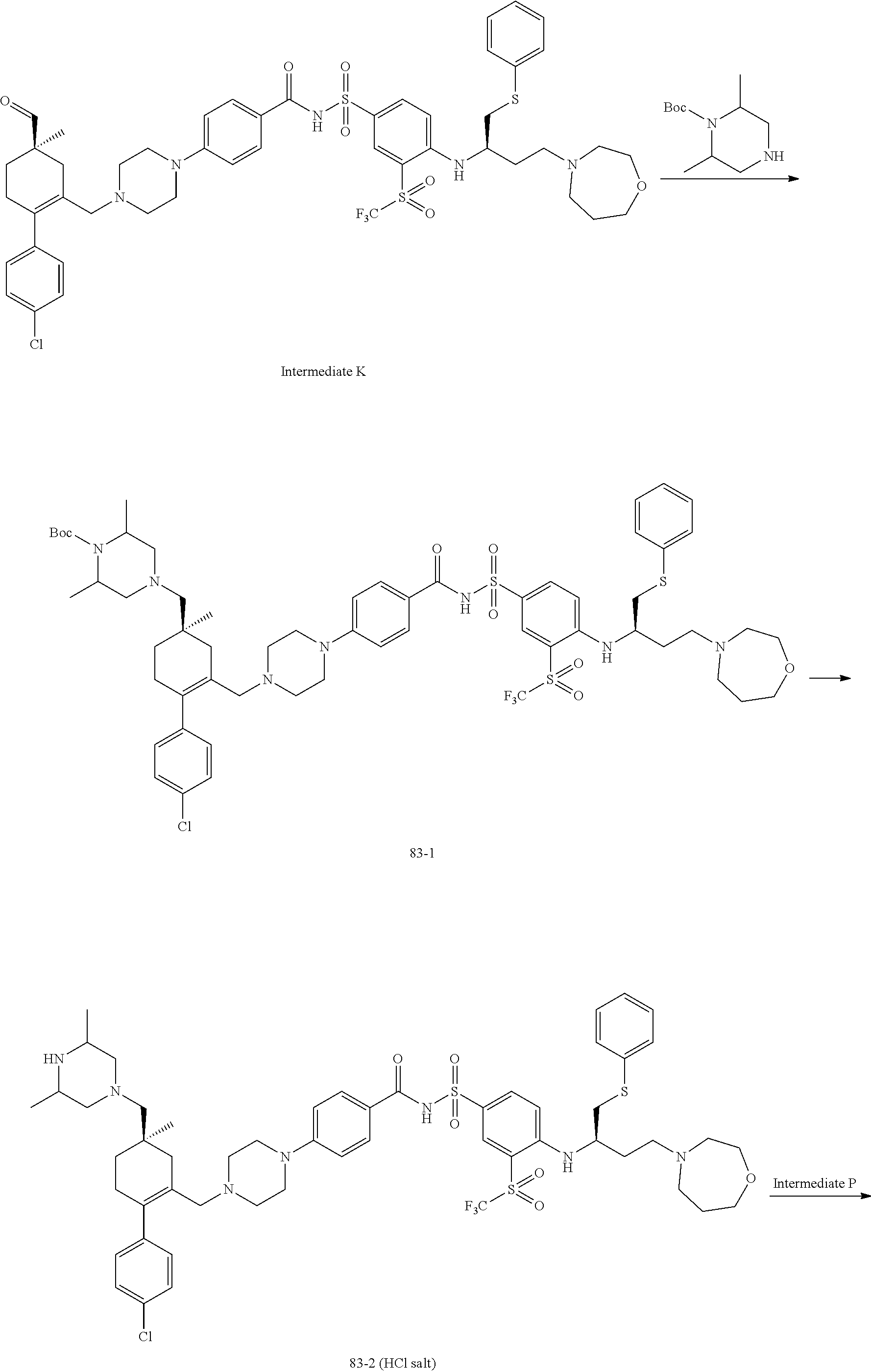

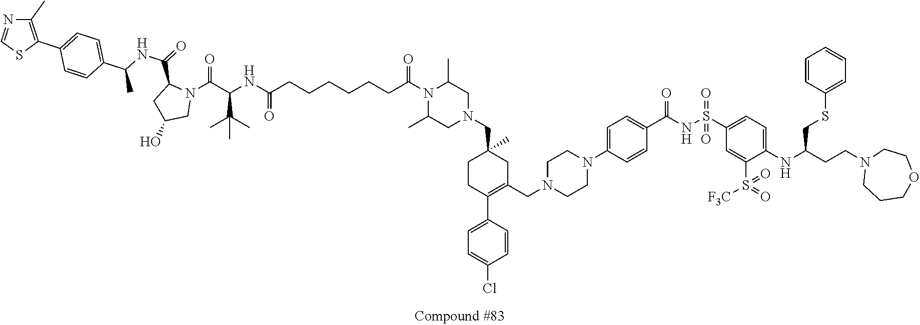 [SMILES: Cc1ncsc1-c1ccc([C@H](C)NC(=O)[C@@H]2C[C@@H](O)CN2C(=O)[C@@H](NC(=O)CCCCCCC(=O)N2C(C)CN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2C)C(C)(C)C)cc1]

Compound #83 was prepared by following General Procedure G. tert-butyl 4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2,6-dimethylpiperazine-1-carboxylate (83-1) (10 mg, 55% yield). ¹H NMR (600 MHz, Chloroform-d) δ 8.36 (d, J=3.8 Hz, 1H), 8.07 (d, J=9.3 Hz, 1H), 7.72 (d, J=9.0 Hz, 2H), 7.39 (dd, J=7.6, 1.9 Hz, 2H), 7.36-7.23 (m, 5H), 7.18-7.09 (m, 1H), 7.03-6.99 (m, 2H), 6.79 (d, 2H), 6.61 (d, J=9.3 Hz, 1H), 4.45 (dt, J=13.4, 1.8 Hz, 1H), 4.29-4.18 (m, 1H), 4.05 (s, 2H), 3.93 (s, 1H), 3.76-3.69 (m, 3H), 3.61 (dt, J=13.5, 1.9 Hz, 1H), 3.34 (dd, J=13.5, 4.6 Hz, 1H), 3.27 (t, J=5.0 Hz, 4H), 3.15 24-3.01 (m, 2H), 2.90-2.56 (m, 8H), 2.45 (dd, J=11.1, 4.2 Hz, 1H), 2.42-2.28 (m, 6H), 2.28-2.18 (m, 3H), 2.15-2.08 (m, 1H), 2.03-1.88 (m, 3H), 1.77 (d, J=7.2 Hz, 1H), 1.67 (dt, J=14.3, 7.5 Hz, 1H), 1.50 (s, 6H), 1.48 (s, 9H), 1.01 (s, 3H) ppm. LC/MS (ESI) m/z 1200.6; [M+H]⁺ calcd for C₅₉H₇₈ClF₃N₇O₈S₃⁺: 1200.47.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((4R)-4′-chloro-4-((3,5-dimethylpiperazin-1-yl)methyl)-4-methyl-3,4,5,6-tetrahydro-[1,1′-biphenyl]-2-yl)methyl)piperazin-1-yl)benzamide hydrochloride (83-2) (12 mg, quantitative yield). LC/MS (ESI) m/z 1100.6; [M+H]⁺ calcd for C₅₄H₇₀ClF₃N₇O₆S₃⁺: 1100.42.

(2S,4R)-1-((2S)-2-(8-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2,6-dimethylpiperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #83) (3.1 mg, 22% yield in two steps). ¹H NMR (600 MHz, CDCl₃) δ 8.70 (s, 1H), 8.35 (d, J=2.2 Hz, 1H), 8.11 (dd, J=9.3, 2.2 Hz, 1H), 7.72 (d, J=8.6 Hz, 2H), 7.53 (d, J=7.9 Hz, 1H), 7.44-7.36 (m, 6H), 7.35-7.24 (m, 5H), 7.18 (d, J=8.6 Hz, 1H), 7.04-6.98 (m, 2H), 6.78 (d, 2H), 6.62 (d, J=9.3 18 Hz, 1H), 6.28 (d, J=8.6 Hz, 1H), 5.12 (p, J=7.1 Hz, 1H), 4.77 (t, J=8.0 Hz, 1H), 4.63 (s, 1H), 4.52 (s, 2H), 4.16 (d, J=11.5 Hz, 1H), 3.98-3.83 (m, 2H), 3.78 (t, J=6.1 Hz, 2H), 3.74-3.65 (m, 2H), 3.60 (d, J=11.1 Hz, 1H), 3.24 (s, 4H), 3.15-3.01 (m, 2H), 2.84 (s, 2H), 2.79-2.55 (m, 7H), 2.55-2.46 (m, 4H), 2.46-2.15 (m, 16H), 2.11 (t, J=11.3 Hz, 2H), 1.99 (d, J=17.0 22 Hz, 1H), 1.89 (s, 2H), 1.77-1.54 (m, 8H), 1.53-1.42 (m, 7H), 1.40-1.24 (m, 6H), 1.10-0.98 (m, 12H) ppm. LC/MS (ESI) m/z 1682.6; [M+H]⁺ calcd for C₈₅H₁₁₂ClF₃N₁₁O₁₁S₄⁺: 1682.71.

### Example 47: Preparation of Compound #88

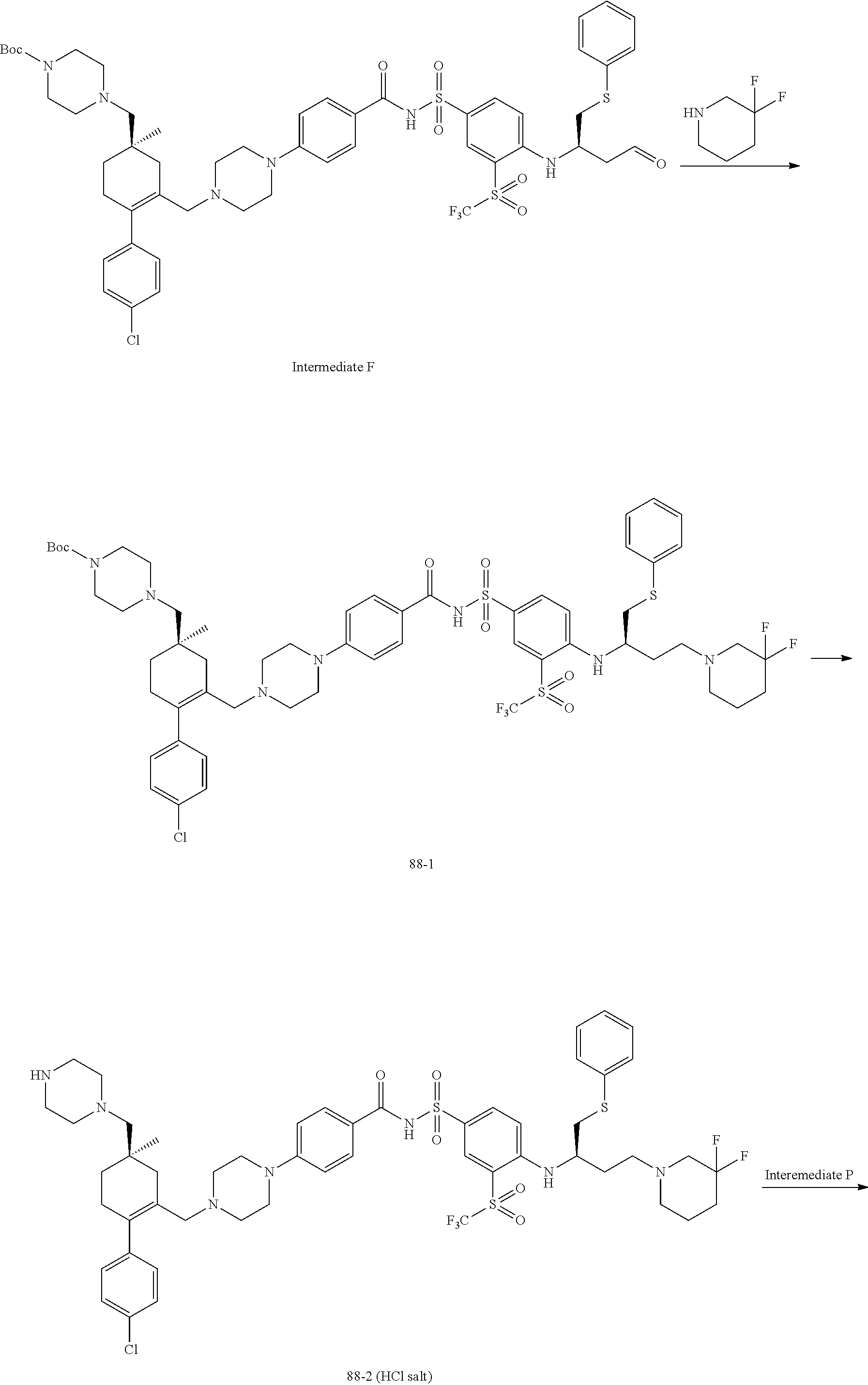

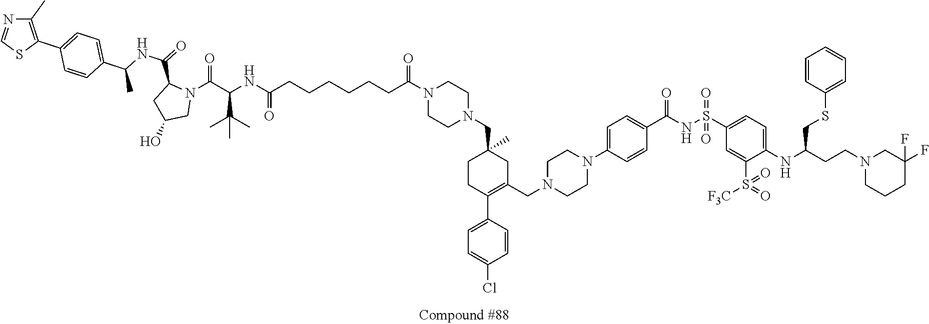 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCC(F)(F)C7)CSc7ccccc7)c(S(=O)(=O)C(F)(F)F)c6)cc5)CC4)C3)CC2)C(C)(C)C)cc1]

Compound #88 was prepared by following General Procedure G. tert-butyl 4-(((R)-4′-chloro-6-((4-(4-(((4-(((R)-4-(3,3-difluoropiperidin-1-yl)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazine-1-carboxylate (88-1) (13 mg, 79% yield). ¹H NMR (600 MHz, Chloroform-d) δ 8.40 (d, J=2.3 Hz, 1H), 8.11 (dd, J=9.2, 2.3 Hz, 1H), 7.66 (d, J=8.6 Hz, 2H), 7.41-7.38 (m, 2H), 7.35-7.24 (m, 5H), 7.02 (dd, J=16.8, 8.6 Hz, 3H), 6.80 (d, J=8.2 Hz, 2H), 6.71 (d, J=9.4 Hz, 1H), 4.03-3.95 (m, 1H), 3.42 (s, 4H), 3.29 (s, 4H), 3.14-3.02 (m, 2H), 2.84 (d, J=15.4 Hz, 1H), 2.66 (q, J=11.2 Hz, 1H), 2.58-2.03 (m, 22H), 1.99-1.82 (m, 2H), 1.77-1.58 (m, 4H), 1.47 (s, 9H), 0.97 (s, 3H) ppm. LC/MS (ESI) m/z 1192.3; [M+H]⁺ calcd for C₅₇H₇₂ClF₅N₇O₇S₃⁺: 1192.43.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-(3,3-difluoropiperidin-1-yl)-1-(phenylthio)butan-2-yl)amino)-3-((trifluoromethyl)sulfonyl)phenyl)sulfonyl)benzamide hydrochloride (88-2) (11 mg, quantitative yield). LC/MS (ESI) m/z 1092.3; [M+H]⁺ calcd for C₅₂H₆₄ClF₅N₇O₅S₃⁺: 1092.37.

(2S,4R)-1-((S)-2-(8-(4-(((R)-4′-chloro-6-((4-(4-(((4-(((R)-4-(3,3-difluoropiperidin-1-yl)-1-(phenylthio)butan-2-yl)amino)-3-((trifluoromethyl)sulfonyl)phenyl)sulfonyl)carbamoyl)phenyl)piperazin-1-yl)methyl)-4-methyl-2,3,4,5-tetrahydro-[1,1′-biphenyl]-4-yl)methyl)piperazin-1-yl)-8-oxooctanamido)-2-(3,3-dimethylbutanoyl)-4-hydroxy-N—((S)-1-(4-(4-methylthiazol-5-yl)phenyl)ethyl)pyrrolidine-2-carboxamide (compound #88) (5.7 mg, 31% yield in two steps). ¹H NMR (600 MHz, Chloroform-d) δ 8.70 (s, 1H), 8.36 (s, 1H), 8.10 (d, J=9.1 Hz, 1H), 7.71 (d, J=8.4 Hz, 2H), 7.55 (d, J=8.1 Hz, 1H), 7.45-7.37 (m, 7H), 7.35-7.22 (m, 5H), 7.06-6.95 (m, 2H), 6.80-6.67 (m, 3H), 6.31-6.21 (m, 1H), 5.12 (p, J=7.0 Hz, 1H), 4.82-4.69 (m, 1H), 4.60 (d, J=8.6 Hz, 1H), 4.52 (s, 1H), 4.15 (d, J=10.9 Hz, 1H), 3.99 (s, 1H), 3.60 (dd, J=11.4, 2.9 Hz, 3H), 3.49-3.18 (m, 7H), 3.09 (qd, J=13.9, 6.0 Hz, 2H), 2.87 (s, 1H), 2.70-2.15 (m, 24H), 2.10 (dd, J=13.4, 8.4 Hz, 2H), 1.88 (s, 3H), 1.75-1.55 (m, 8H), 1.50 (d, J=6.9 Hz, 4H), 1.31 (d, J=24.8 Hz, 6H), 1.18 (t, J=7.1 Hz, 1H), 1.11 (t, J=7.1 Hz, 1H), 1.08 (s, 9H), 0.97 (s, 3H) ppm. LC/MS (ESI) m/z 1674.7; [M+H]⁺ calcd for C₈₃H₁₀₆ClF₅N₁₁O₁₀S₄⁺: 1674.66.

### Example 48: Preparation of Compound #89

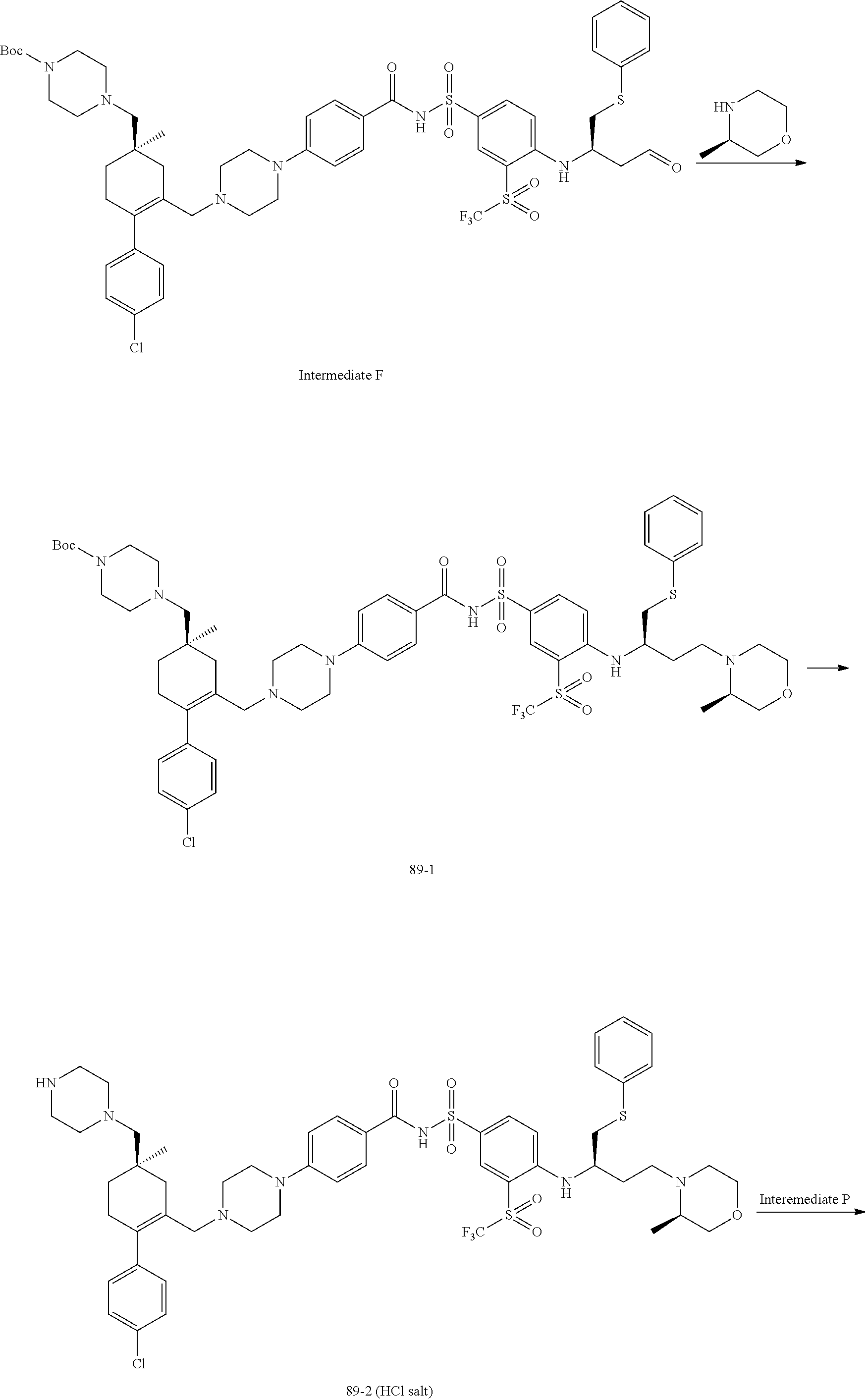

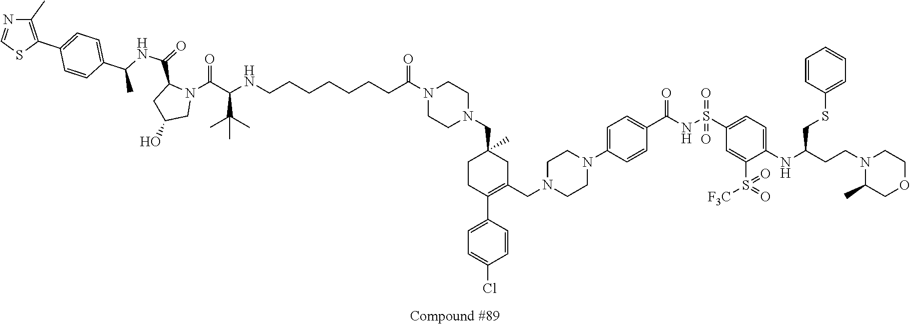 [SMILES: Cc1ncsc1-c1ccc([C@H](C)NC(=O)[C@@H]2C[C@@H](O)CN2C(=O)[C@@H](NCCCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC[C@H]7C)CSc7ccccc7)c(S(=O)(=O)C(F)(F)F)c6)cc5)CC4)C3)CC2)C(C)(C)C)cc1]

Compound #89 was prepared by following General Procedure G. tert-butyl 4-(((R)-4′-chloro-4-methyl-6-((4-(4-(((4-(((R)-4-((R)-3-methylmorpholino)-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (89-1) (11 mg, 68% yield). ¹H NMR (600 MHz, Chloroform-d) δ 8.38 (d, J=2.3 Hz, 1H), 8.14 (dd, J=9.3, 2.3 Hz, 1H), 7.67 (d, 2H), 7.41 (dt, J=6.4, 1.3 Hz, 2H), 7.37-7.25 (m, 4H), 7.10 (d, J=8.1 Hz, 1H), 7.03-6.98 (m, 2H), 6.79 (d, J=8.8 Hz, 2H), 6.58 (d, J=9.3 Hz, 1H), 3.84 (q, J=6.8 Hz, 1H), 3.76 (dt, J=11.4, 3.6 Hz, 1H), 3.69-3.61 (m, 2H), 3.42 (t, J=4.9 Hz, 4H), 3.34-3.24 (m, 5H), 3.17-3.04 (m, 2H), 2.86 (s, 2H), 2.79 (dt, J=13.5, 6.9 Hz, 1H), 2.69 (dt, J=12.1, 3.2 Hz, 1H), 2.57-2.46 (m, 4H), 2.45-2.27 (m, 7H), 2.27-2.12 (m, 6H), 2.12-2.01 (m, 1H), 1.95 (d, J=17.3 Hz, 1H), 1.80-1.69 (m, 1H), 1.66-1.57 (m, 1H), 1.47 (s, 9H), 0.98-0.93 (m, 6H) ppm. LC/MS (ESI) m/z 1172.6; [M+H]⁺ calcd for Cs₇H₇₄ClF₃N₇O₈S₃⁺: 1172.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-((R)-3-methylmorpholino)-1-(phenylthio)butan-2-yl)amino)-3-((trifluoromethyl)sulfonyl)phenyl)sulfonyl)benzamide hydrochloride (89-1) (10 mg, quantitative yield). LC/MS (ESI) m/z 1072.4; [M+H]⁺ calcd for Cs₂H₆₆ClF₃N₇O₆S₃⁺: 1072.39.

(2S,4R)-1-((S)-2-(8-(4-(((R)-4′-chloro-4-methyl-6-((4-(4-(((4-(((R)-4-((R)-3-methylmorpholino)-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #89) (8.8 mg, 57% yield in two steps). ¹H NMR (600 MHz, Chloroform-d) δ 8.70 (s, 1H), 8.35 (d, J=2.3 Hz, 1H), 8.14 (d, 1H), 7.71 (d, J=8.4 Hz, 2H), 7.49 (d, J=7.9 Hz, 1H), 7.44-7.37 (m, 6H), 7.35-7.24 (m, 5H), 7.08 (d, J=8.3 Hz, 1H), 7.03-6.98 (m, 2H), 6.77 (d, 2H), 6.58 (d, J=9.3 Hz, 1H), 6.32 (s, 1H), 5.11 (p, J=7.0 Hz, 1H), 4.76 (t, J=8.0 Hz, 1H), 4.64 (d, J=8.8 Hz, 1H), 4.52 (s, 1H), 4.15 (dd, J=9.2, 5.5 Hz, 1H), 3.87-3.80 (m, 1H), 3.79-3.70 (m, 1H), 3.69-3.51 (m, 4H), 3.44 (t, J=5.3 Hz, 2H), 3.32-3.21 (m, 5H), 3.15-3.04 (m, 2H), 2.87 (s, 2H), 2.79 (dt, J=13.7, 7.0 Hz, 1H), 2.68 (d, J=11.7 Hz, 1H), 2.63-2.47 (m, 7H), 2.46-2.34 (m, 5H), 2.34-2.15 (m, 12H), 2.14-2.00 (m, 2H), 1.92 (d, J=17.2 Hz, 1H), 1.73 (dq, J=14.3, 7.1 Hz, 1H), 1.69-1.54 (m, 6H), 1.51-1.42 (m, 4H), 1.40-1.22 (m, 5H), 1.07 (s, 9H), 0.97 (s, 3H), 0.95 (d, J=6.4 Hz, 3H) ppm. LC/MS (ESI) m/z 1654.4; [M+H]⁺ calcd for C₈₃H₁₀₈ClF₃N₁₁O₁₁S₄⁺: 1654.67.

### Example 49: Preparation of Compound #91

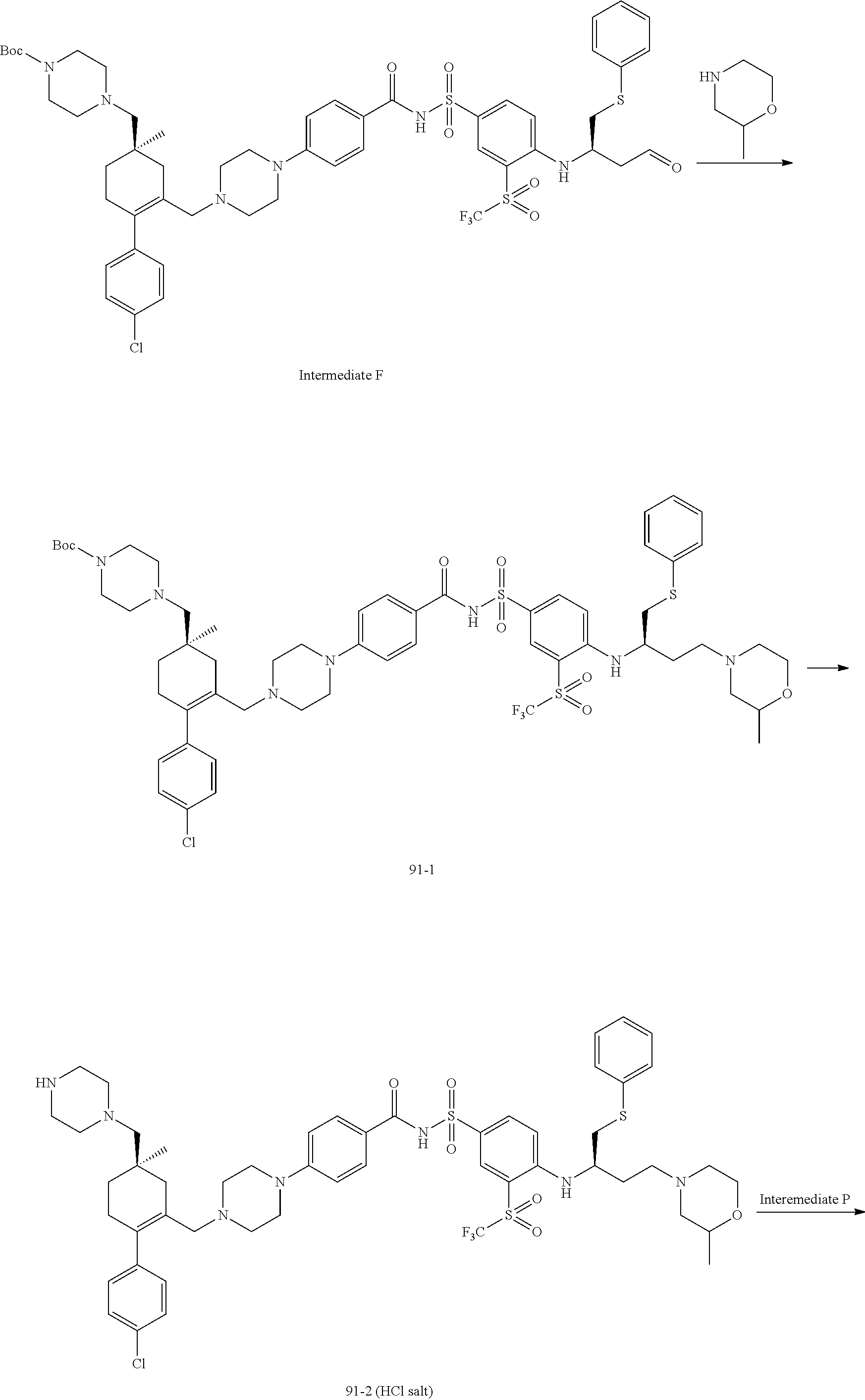

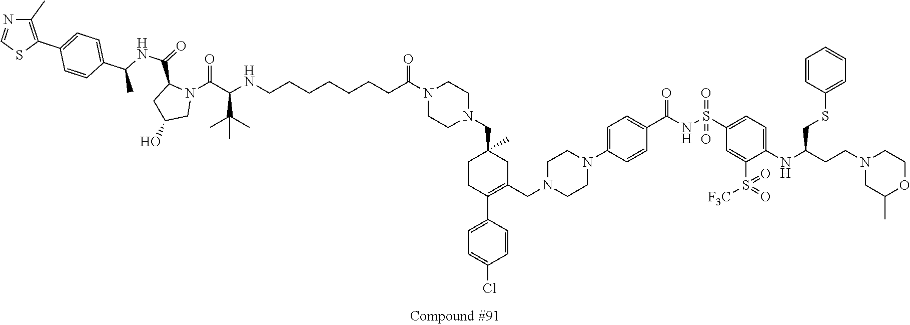 [SMILES: Cc1ncsc1-c1ccc([C@H](C)NC(=O)[C@@H]2C[C@@H](O)CN2C(=O)[C@@H](NCCCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC(C)C7)CSc7ccccc7)c(S(=O)(=O)C(F)(F)F)c6)cc5)CC4)C3)CC2)C(C)(C)C)cc1]

Compound #91 was prepared by following General Procedure G. tert-butyl 4-(((4R)-4′-chloro-4-methyl-6-((4-(4-(((4-(((2R)-4-(2-methylmorpholino)-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (91-1) (9.4 mg, 58% yield). ¹H NMR (600 MHz, Chloroform-d) δ 8.38 (d, J=2.3 Hz, 1H), 8.13 (dd, J=9.3, 2.3 Hz, 1H), 7.66 (d, J=8.6 Hz, 2H), 7.42-7.38 (m, 2H), 7.36-7.31 (m, 2H), 7.32-7.25 (m, 2H), 7.08 (d, J=8.6 Hz, 1H), 7.03-6.98 (m, 2H), 6.80 (d, J=9.0 Hz, 2H), 6.63 (d, J=9.4 Hz, 1H), 3.96-3.89 (m, 1H), 3.85 (ddd, J=11.4, 3.4, 1.5 Hz, 1H), 3.65-3.56 (m, 2H), 3.42 (t, J=4.9 26 Hz, 4H), 3.29 (t, J=5.3 Hz, 4H), 3.12 (dd, J=13.9, 5.0 Hz, 1H), 3.04 (dd, J=13.9, 7.2 Hz, 1H), 2.84 (s, 2H), 2.71 (d, J=11.2 Hz, 1H), 2.58 (d, J=11.2 Hz, 1H), 2.55-2.47 (m, 4H), 2.47-2.27 (m, 7H), 2.28-2.11 (m, 6H), 2.09-2.02 (m, 1H), 1.94 (d, J=17.3 Hz, 1H), 1.82 (t, J=10.6 Hz, 1H), 1.75-1.56 (m, 2H), 1.47 (s, 9H), 1.13 (d, J=6.3 Hz, 3H), 0.96 (s, 3H) ppm. LC/MS (ESI) m/z 1172.5; [M+H]⁺ calcd for C₅₇H₇₄ClF₃N₇O₈S₃⁺: 1172.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((2R)-4-(2-methylmorpholino)-1-(phenylthio)butan-2-yl)amino)-3-((trifluoromethyl)sulfonyl)phenyl)sulfonyl)benzamide hydrochloride (91-2) (8 8 mg, quantitative yield). LC/MS (ESI) m/z 1072.5; [M+H]⁺ calcd for C₅₂H₆₆ClF₃N₇O₆S₃⁺: 1072.39.

(2S,4R)-1-((2S)-2-(8-(4-(((4R)-4′-chloro-4-methyl-6-((4-(4-(((4-(((2R)-4-(2-methylmorpholino)-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #91) (7.7 mg, 58% yield in two steps). ¹H NMR (600 MHz, Chloroform-d) δ 8.70 (s, 1H), 8.35 (d, J=2.3 Hz, 1H), 8.14 (d, J=9.2 Hz, 1H), 7.70 (d, J=8.2 Hz, 2H), 7.49 (d, J=7.8 Hz, 1H), 7.44-7.37 (m, 6H), 7.36-7.25 (m, 5H), 7.09-7.04 (m, 1H), 7.02-6.98 (m, 2H), 6.78 (d, J=8.7 Hz, 2H), 6.63 (d, J=9.5 Hz, 1H), 6.30 (s, 1H), 5.11 (q, J=7.0 Hz, 1H), 4.76 (t, J=8.1 Hz, 1H), 4.64 (d, J=8.8 Hz, 1H), 4.52 (s, 1H), 4.15 (dd, J=9.3, 5.9 Hz, 1H), 3.92 (s, 1H), 3.88-3.82 (m, 1H), 3.65-3.53 (m, 5H), 3.43 (d, J=5.6 Hz, 2H), 3.25 (t, J=5.5 Hz, 4H), 3.12 (dd, J=13.9, 5.0 Hz, 1H), 3.04 (dd, J=13.9, 7.2 Hz, 1H), 2.85 (s, 2H), 2.70 (d, J=11.3 Hz, 1H), 2.63-2.48 (m, 9H), 2.37 (q, J=7.7, 6.2 Hz, 5H), 2.32-2.18 (m, 10H), 2.18-2.00 (m, 2H), 1.92 (d, J=17.3 Hz, 1H), 1.81 (t, J=10.6 Hz, 1H), 1.74-1.56 (m, 7H), 1.53-1.42 (m, 4H), 1.38-1.29 (m, 5H), 1.13 (d, J=6.2 Hz, 3H), 1.07 (s, 9H), 0.97 (s, 3H) ppm. LC/MS (ESI) m/z 1654.8; [M+H]⁺ calcd for C₈₃H₁₀₈ClF₃N₁₁O₁₁S₄⁺: 1654.67.

### Example 50: Preparation of Compound #92

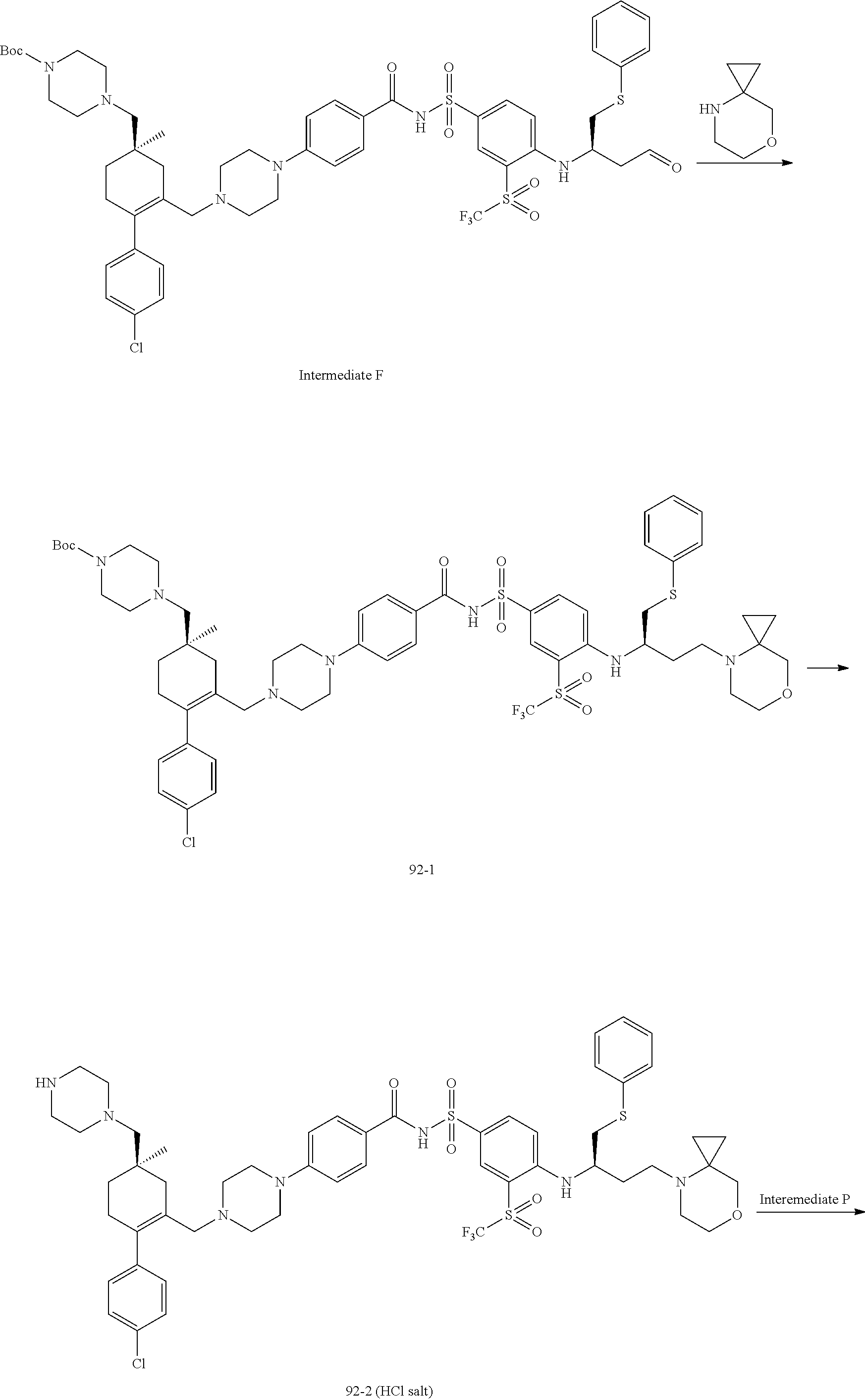

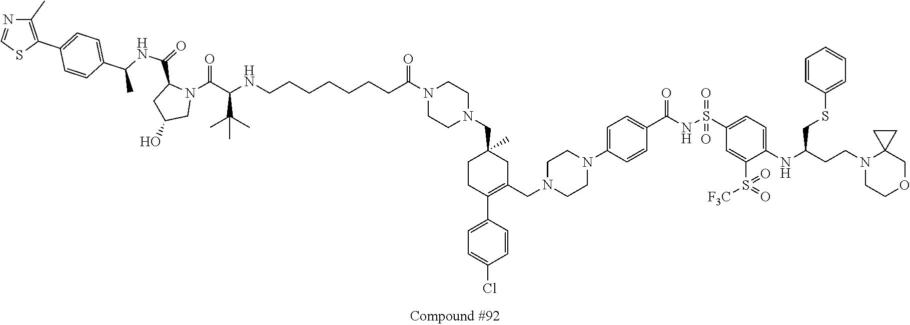 [SMILES: Cc1ncsc1-c1ccc([C@H](C)NC(=O)[C@@H]2C[C@@H](O)CN2C(=O)[C@@H](NCCCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC78CC8)CSc7ccccc7)c(S(=O)(=O)C(F)(F)F)c6)cc5)CC4)C3)CC2)C(C)(C)C)cc1]

Compound #92 was prepared by following General Procedure G. tert-butyl 4-(((R)-4′-chloro-4-methyl-6-((4-(4-(((4-(((R)-1-(phenylthio)-4-(7-oxa-4-azaspiro[2.5]octan-4-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (92-1) (8.1 mg, 50% yield). ¹H NMR (600 MHz, Chloroform-d) δ 8.38 (t, J=3.0 Hz, 1H), 8.11 (dd, J=9.2, 2.3 Hz, 1H), 7.66 (d, J=8.7 Hz, 2H), 7.43-7.38 (m, 2H), 7.37-7.24 (m, 4H), 7.08 (d, J=8.4 Hz, 1H), 7.03-6.97 (m, 2H), 6.80 (d, J=8.9 Hz, 2H), 6.47 (d, J=9.3 Hz, 1H), 3.86-3.78 (m, 1H), 3.77-3.70 (m, 1H), 3.68-3.60 (m, 1H), 3.50 (d, J=12.2 Hz, 1H), 3.42 (t, J=4.7, 0.0 Hz, 4H), 3.29 (t, J=0.0 Hz, 4H), 3.07 (dd, J=13.8, 4.8, 0.0 Hz, 1H), 3.02-2.92 (m, 2H), 2.86 (p, J=8.4, 6.2, 2.7 Hz, 4H), 2.69-2.61 (m, 1H), 2.51 (s, 4H), 2.45-2.27 (m, 7H), 2.27-2.19 (m, 4H), 2.16 (d, J=17.3 Hz, 1H), 2.08-1.99 (m, 1H), 1.95 (d, J=17.3 Hz, 1H), 1.68-1.58 (m, 2H), 1.47 (s, 9H), 0.96 (s, 3H), 0.62-0.53 (m, 1H), 0.49-0.43 (m, 1H), 0.43-0.36 (m, 2H) ppm. LC/MS (ESI) n/z 1184.5; [M+H]⁺ calcd for C₅₈H₇₄ClF₃N₇O₈S₃⁺: 1184.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-1-(phenylthio)-4-(7-oxa-4-azaspiro[2.5]octan-4-yl)butan-2-yl)amino)-3-((trifluoromethyl)sulfonyl)phenyl)sulfonyl)benzamide hydrochloride (91-2) (7.5 mg, quantitative yield). LC/MS (ESI) n/z 1084.5; [M+H]⁺ calcd for C₅₃H₆₆ClF₃N₇O₆S₃⁺: 1084.39.

(2S,4R)-1-((S)-2-(8-(4-(((R)-4′-chloro-4-methyl-6-((4-(4-(((4-(((R)-1-(phenylthio)-4-(7-oxa-4-azaspiro[2.5]octan-4-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #92) (4.8 mg, 42% yield in two steps). ¹H NMR (600 MHz, Chloroform-d) δ 8.70 (s, 1H), 8.35 (d, J=2.2 Hz, 1H), 8.15-8.09 (m, 1H), 7.69 (d, J=8.6 Hz, 2H), 7.50 (d, J=8.1 Hz, 1H), 7.44-7.38 (m, 6H), 7.36-7.32 (m, 2H), 7.33-7.26 (m, 3H), 7.07 (d, J=8.4 Hz, 1H), 7.02-6.98 (m, 2H), 6.78 (d, J=8.8 Hz, 2H), 6.48 (d, J=9.3 Hz, 1H), 6.29 (d, J=12.6 Hz, 1H), 5.12 (p, J=7.0 Hz, 1H), 4.77 (t, J=8.1 Hz, 1H), 4.64 (d, J=8.8 Hz, 1H), 4.52 (s, 1H), 4.16 (d, J=11.5 Hz, 1H), 3.86-3.78 (m, 1H), 3.78-3.71 (m, 1H), 3.69-3.47 (m, 3H), 3.44 (s, 2H), 3.32-3.20 (m, 5H), 3.07 (dd, J=13.9, 4.8 Hz, 1H), 3.02-2.91 (m, 2H), 2.89-2.78 (m, 4H), 2.70-2.46 (m, 9H), 2.45-2.33 (m, 4H), 2.33-2.17 (m, 10H), 2.10 (dd, J=13.6, 8.3 Hz, 1H), 2.06-1.98 (m, 1H), 1.92 (d, J=17.2 Hz, 1H), 1.69-1.54 (m, 7H), 1.51-1.41 (m, 4H), 1.31 (d, J=7.4 Hz, 5H), 1.07 (s, 9H), 0.97 (s, 3H), 0.58 (q, J=7.0 Hz, 1H), 0.48-0.42 (m, 1H), 0.40 (t, J=7.7 Hz, 2H) ppm. LC/MS (ESI) m/z 1666.8; [M+H]⁺ calcd for Cs₄H₁₀₈ClF₃N₁₁O₁₁S₄⁺: 1666.67.

### Example 51: Preparation of Compound #93

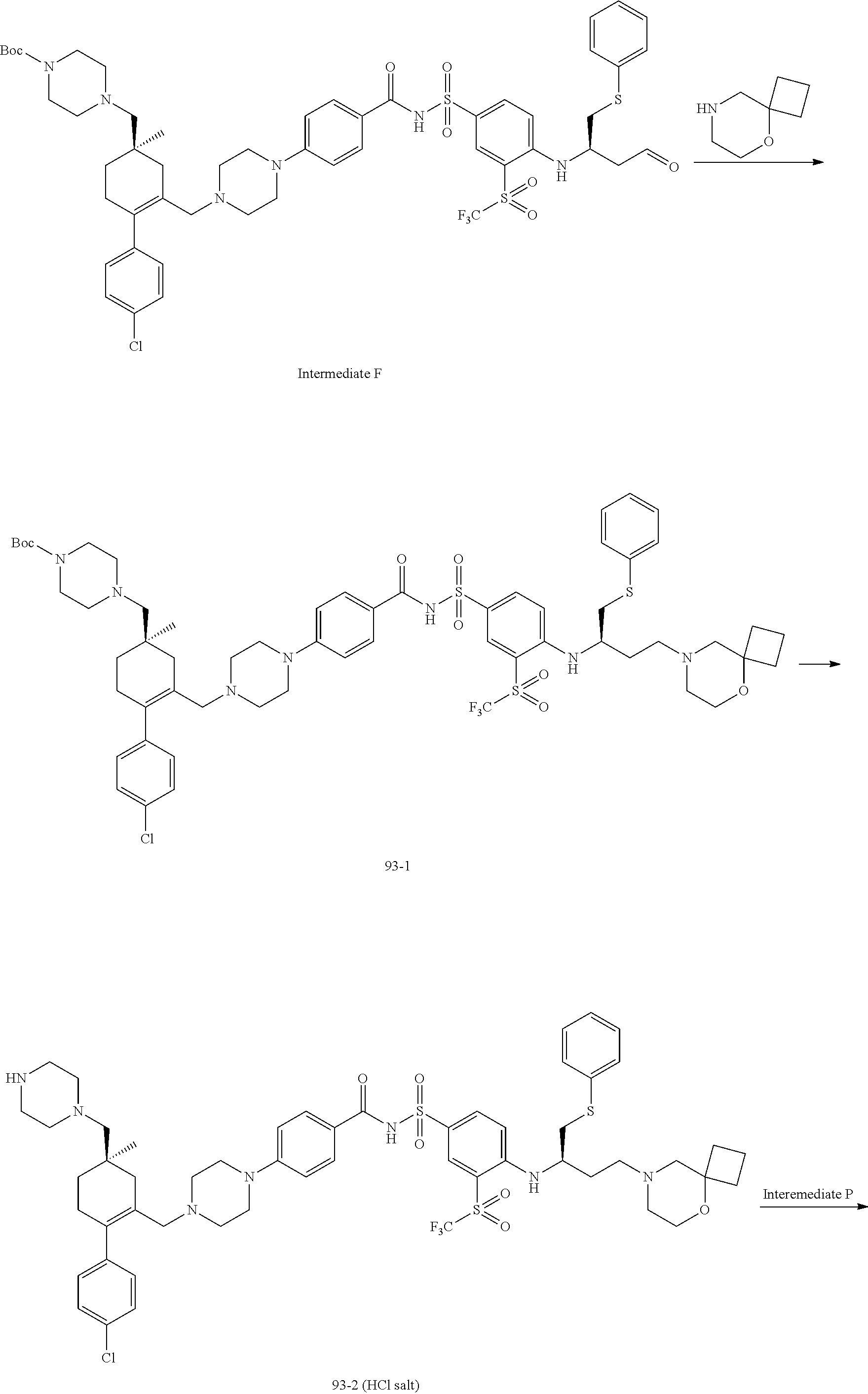

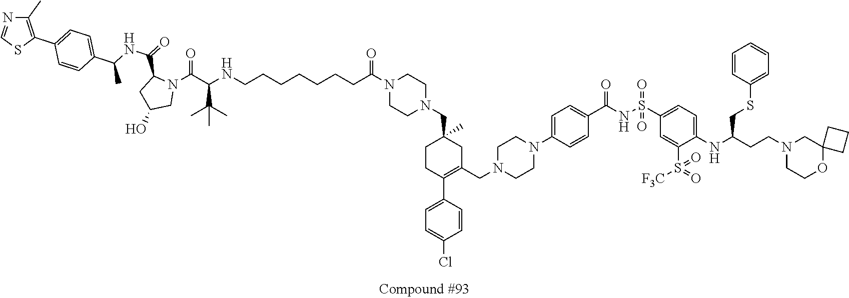 [SMILES: Cc1ncsc1-c1ccc([C@H](C)NC(=O)[C@@H]2C[C@@H](O)CN2C(=O)[C@@H](NCCCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOC8(CCC8)C7)CSc7ccccc7)c(S(=O)(=O)C(F)(F)F)c6)cc5)CC4)C3)CC2)C(C)(C)C)cc1]

Compound #93 was prepared by following General Procedure G. tert-butyl 4-(((R)-4′-chloro-4-methyl-6-((4-(4-(((4-(((R)-1-(phenylthio)-4-(5-oxa-8-azaspiro[3.5]nonan-8-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (93-1) (9.2 mg, 56% yield). ¹H NMR (600 MHz, Chloroform-d) δ 8.39 (d, J=2.3 Hz, 1H), 8.14 (dd, J=9.3, 2.3 Hz, 1H), 7.65 (d, J=8.8 Hz, 2H), 7.41-7.36 (m, 2H), 7.35-7.24 (m, 4H), 7.01 (dd, J=11.6, 8.7 Hz, 3H), 6.80 (d, 2H), 6.68 (d, J=9.4 Hz, 1H), 3.98 (s, 1H), 3.62-3.55 (m, 2H), 3.42 (t, J=5.0 Hz, 4H), 3.29 (t, J=5.2 Hz, 4H), 3.11 (qd, J=13.9, 6.1 Hz, 2H), 2.85 (s, 2H), 2.51 (s, 4H), 2.47-2.18 (m, 15H), 2.18-2.10 (m, 2H), 2.05-1.89 (m, 5H), 1.84-1.76 (m, 1H), 1.71-1.52 (m, 3H), 1.47 (s, 9H), 0.96 (s, 3H) ppm. LC/MS (ESI) m/z 1198.6; [M+H]⁺ calcd for C₅₉H₇₆ClF₃N₇O₈S₃⁺: 1198.46.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-1-(phenylthio)-4-(5-oxa-8-azaspiro[3.5]nonan-8-yl)butan-2-yl)amino)-3-((trifluoromethyl)sulfonyl)phenyl)sulfonyl)benzamide hydrochloride (93-2) (8.5 mg, quantitative yield). LC/MS (ESI) m/z 1098.6; [M+H]⁺ calcd for C₅₃H₆₆ClF₃N₇O₆S₃⁺: 1098.40.

(2S,4R)-1-((S)-2-(8-(4-(((R)-4′-chloro-4-methyl-6-((4-(4-(((4-(((R)-1-(phenylthio)-4-(5-oxa-8-azaspiro[3.5]nonan-8-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #93) (5.9 mg, 48% yield in two steps). ¹H NMR (600 MHz, Chloroform-d) δ 8.70 (s, 1H), 8.36 (d, J=2.3 Hz, 1H), 8.14 (dd, J=9.3, 2.2 Hz, 1H), 7.69 (d, J=8.4 Hz, 2H), 7.49 (d, J=7.8 Hz, 1H), 7.44-7.36 (m, 6H), 7.35-7.23 (m, 5H), 7.01 (dd, J=8.7, 2.2 Hz, 3H), 6.78 (d, J=8.9 Hz, 2H), 6.68 (d, J=9.4 Hz, 1H), 6.32 (d, J=8.3 Hz, 1H), 5.12 (p, J=7.1 Hz, 1H), 4.76 (t, J=8.1 Hz, 1H), 4.64 (d, J=8.8 Hz, 1H), 4.52 (d, J=4.7 Hz, 1H), 4.16 (d, J=11.4 Hz, 1H), 3.98 (s, 1H), 3.65-3.51 (m, 5H), 3.43 (d, J=5.2 Hz, 2H), 3.25 (t, J=5.5 22 Hz, 4H), 3.11 (qd, J=13.9, 6.1 Hz, 2H), 2.85 (s, 2H), 2.64-2.46 (m, 8H), 2.43-2.33 (m, 8H), 2.33-2.17 (m, 10H), 2.16-2.06 (m, 2H), 2.05-1.96 (m, 2H), 1.96-1.88 (m, 3H), 1.81 (qd, J 24=10.0, 4.9 Hz, 1H), 1.71-1.52 (m, 9H), 1.51-1.43 (m, 4H), 1.38-1.23 (m, 5H), 1.07 (s, 9H), 0.97 (s, 3H) ppm. LC/MS (ESI) m/z 1680.8; [M+H]⁺ calcd for C₈₅H₁₁₀ClF₃N₁₁O₁₁S₄⁺: 1680.69.

### Example 52: Preparation of Compound #95

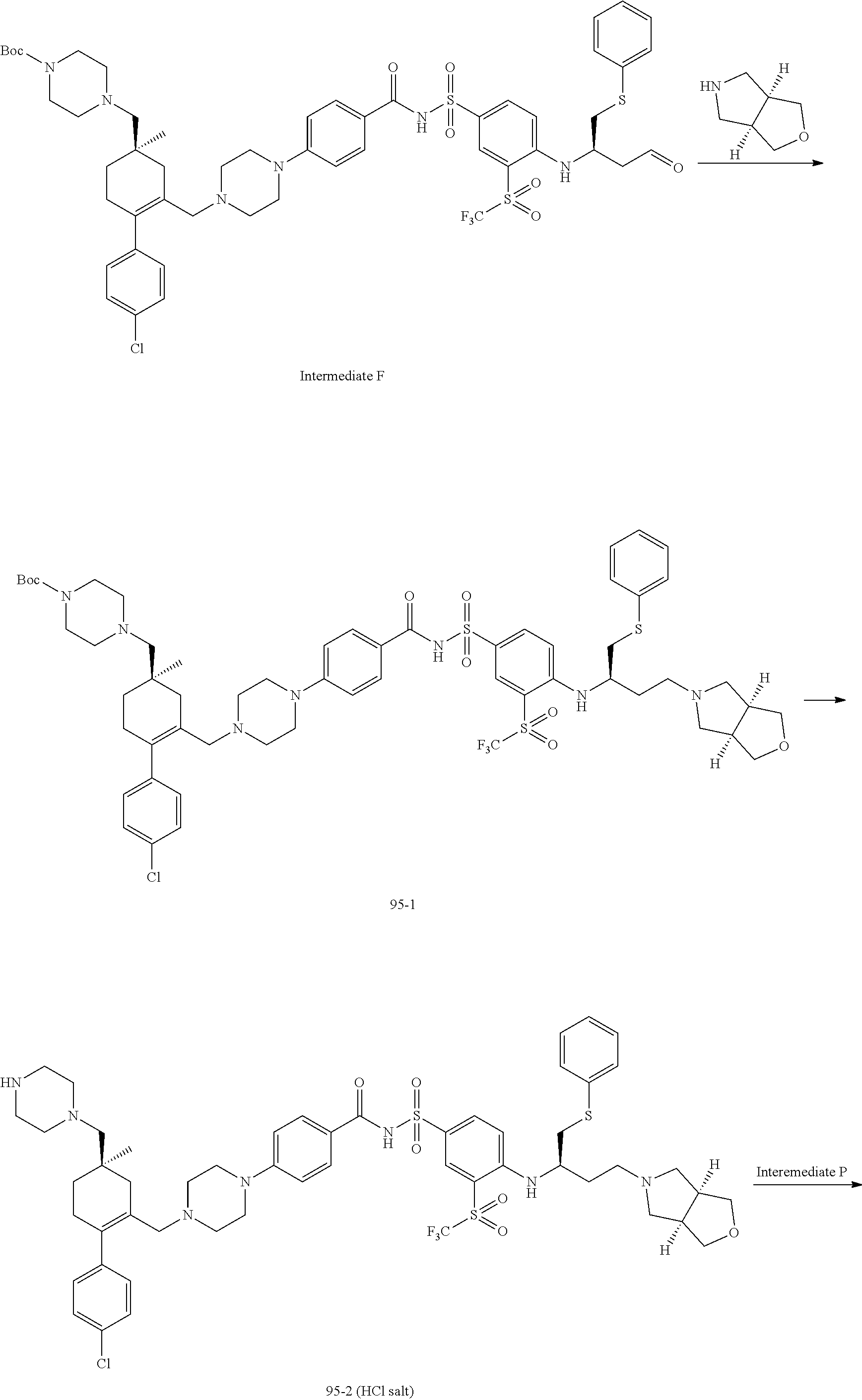 [SMILES: CC(C)(C)OC(=O)N1CCN(C[C@]2(C)CCC(c3ccc(Cl)cc3)=C(CN3CCN(c4ccc(C(=O)NS(=O)(=O)c5ccc(N[C@H](CC=O)CSc6ccccc6)c(S(=O)(=O)C(F)(F)F)c5)cc4)CC3)C2)CC1 ; C1NC[C@H]2COC[C@@H]12 ; CC(C)(C)OC(=O)N1CCN(C[C@]2(C)CCC(c3ccc(Cl)cc3)=C(CN3CCN(c4ccc(C(=O)NS(=O)(=O)c5ccc(N[C@H](CCN6C[C@H]7COC[C@H]7C6)CSc6ccccc6)c(S(=O)(=O)C(F)(F)F)c5)cc4)CC3)C2)CC1 ; C[C@@]1(CN2CCNCC2)CCC(c2ccc(Cl)cc2)=C(CN2CCN(c3ccc(C(=O)NS(=O)(=O)c4ccc(N[C@H](CCN5C[C@H]6COC[C@H]6C5)CSc5ccccc5)c(S(=O)(=O)C(F)(F)F)c4)cc3)CC2)C1]

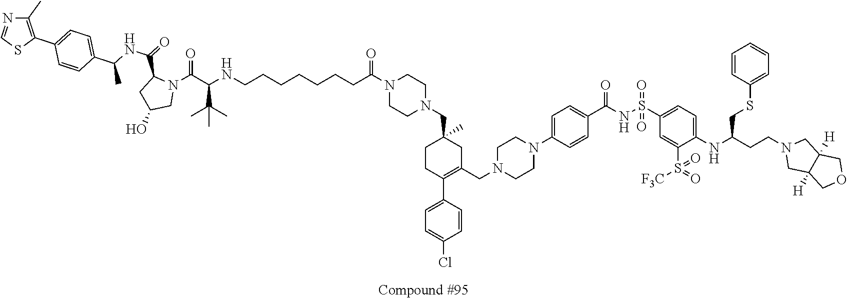 [SMILES: Cc1ncsc1-c1ccc([C@H](C)NC(=O)[C@@H]2C[C@@H](O)CN2C(=O)[C@@H](NCCCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7C[C@H]8COC[C@H]8C7)CSc7ccccc7)c(S(=O)(=O)C(F)(F)F)c6)cc5)CC4)C3)CC2)C(C)(C)C)cc1]

Compound #95 was prepared by following General Procedure G. tert-butyl 4-(((R)-4′-chloro-4-methyl-6-((4-(4-(((4-(((R)-1-(phenylthio)-4-((3aR,6aS)-tetrahydro-1H-furo[3,4-c]pyrrol-5(3H)-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (95-1) (9.0 mg, 55% yield). ¹H NMR (600 MHz, Chloroform-d) δ 8.35 (d, J=2.3 Hz, 1H), 8.03 (dd, J=9.1, 2.3 Hz, 1H), 7.75 (d, J=8.6 Hz, 2H), 7.37 (dt, J=6.2, 1.3 Hz, 2H), 7.33-7.27 (, 3H), 7.26-7.22 (m, 1H), 7.06 (d, J=8.5 Hz, 1H), 7.03-6.98 (m, 2H), 6.79 (d, J=8.6 Hz, 2H), 6.65 (d, J=9.2 Hz, 12=4.8 Hz, 4H), 3.27 (t, 4H), 3.16-3.01 (m, 3H), 3.00-2.89 (m, 3H), 2.85 (s, 2H), 2.75-2.65 (m, 1H), 2.59-2.47 (m, 5H), 2.45-2.18 (m, 12H), 2.18-2.08 (m, 2H), 1.95 (d, J=17.3 Hz, 1H), 1.82 (dd, J=13.9, 7.3 Hz, 1H), 1.66-1.57 (m, 1H), 1.47 (s, 9H), 0.96 (s, 3H) ppm. LC/MS (ESI) m/z 1184.6; [M+H]⁺ calcd for C₅₈H₇₄ClF₃N₇O₈S₃⁺: 1184.44.

4-(4-(((R)-4′-chloro-4-methyl-4-(piperazin-1-ylmethyl)-3,4,5,6-tetrahydro-[1,1′-biphenyl]-2-yl)methyl)piperazin-1-yl)-N-((4-(((R)-1-(phenylthio)-4-((3aR,6aS)-tetrahydro-1H-furo[3,4-c]pyrrol-5(3H)-yl)butan-2-yl)amino)-3-((trifluoromethyl)sulfonyl)phenyl)sulfonyl)benzamide hydrochloride (93-2) (10 mg, quantitative yield). LC/MS (ESI) m/z 1084.6; [M+H]⁺ calcd for Cs₃H₆₆ClF₃N₇O₆S₃⁺: 1084.39.

(2S,4R)-1-((S)-2-(8-(4-(((R)-4′-chloro-4-methyl-6-((4-(4-(((4-(((R)-1-(phenylthio)-4-((3aR,6aS)-tetrahydro-1H-furo[3,4-c]pyrrol-5(3H)-yl)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (compound #95) (7.7 mg, 61% yield in two steps). ¹H NMR (600 MHz, Chloroform-d) δ 8.70 (s, 1H), 8.34 (s, 1H), 8.07 (s, 1H), 7.79-7.70 (m, 2H), 7.50 (d, J=7.9 Hz, 1H), 7.44-7.35 (m, 8H), 7.30 (s, 6H), 7.03-6.98 (m, 2H), 6.78 (d, J=8.5 Hz, 2H), 6.66 (d, J=8.8 Hz, 1H), 6.32 (s, 1H), 5.12 (q, J=7.2 Hz, 1H), 4.76 (t, J=7.9 Hz, 1H), 4.64 (d, J=8.8 Hz, 1H), 4.52 (s, 1H), 4.14 (q, J=6.6, 5.6 Hz, 1H), 3.94 (s, 1H), 3.73 (s, 2H), 3.65-3.48 (m, 5H), 3.44 (t, J=5.2 Hz, 2H), 3.25 (t, J=5.4 Hz, 4H), 3.11 (dd, J=13.9, 4.9 Hz, 1H), 3.04 (dd, J=13.9, 6.9 Hz, 1H), 2.97-2.75 (m, 5H), 2.53 (s, 9H), 2.44-2.32 (m, 5H), 2.32-2.16 (m, 10H), 2.14-2.04 (m, 2H), 1.92 (d, J=17.3 Hz, 1H), 1.78 (s, 1H), 1.67-1.55 (m, 6H), 1.53-1.40 (m, 4H), 1.36-1.24 (m, 5H), 1.06 (s, 9H), 0.97 (s, 3H) ppm. LC/MS (ESI) m/z 1665.5; [M+H]+ calcd for Cs₄H₁₀₇ClF₃N₁₁O₁₁S₄⁺: 1665.67.

### Example 53: Preparation of Compounds #96 and #106-107

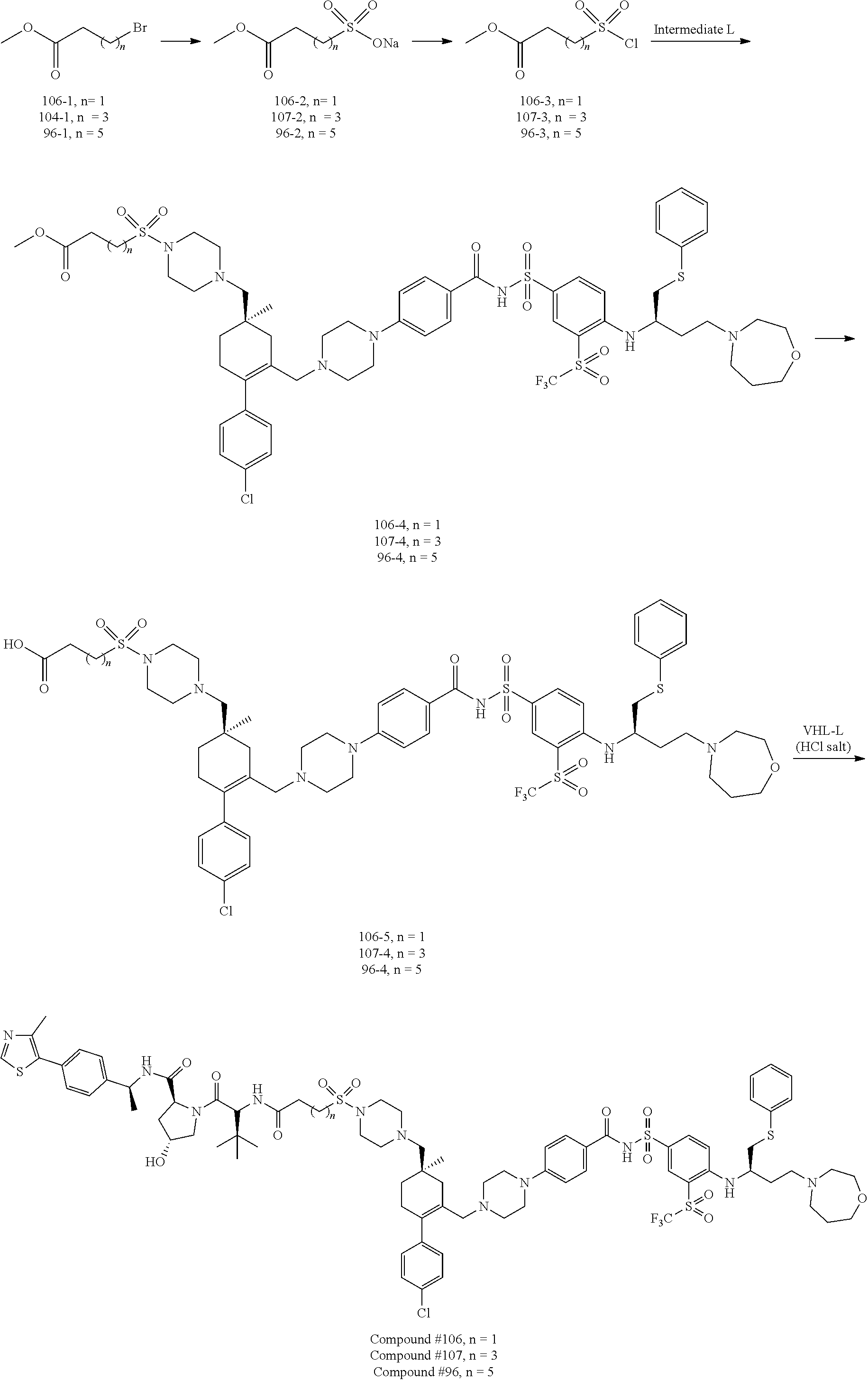

Preparation of methyl 3-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)propanoate (106-4), methyl 5-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)pentanoate (107-4), and methyl 7-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)heptanoate (96-4): A suspension of 106-1, 107-1, or 96-1 (1.0 equiv.) and Na₂SO₃ (2.0 equiv.) in EtOH/H₂O was refluxed for 2 hours. The resulting mixture was concentrated. The residue was extracted with boiling ethanol in a Soxhlet extraction apparatus overnight. The solution was transferred to a round-flask and solvents were removed under vacuum to afford the corresponding sodium sulfonate 106-2, 107-2, and 96-2, which were used directly in the next step.

Under an argon atmosphere, SOCl₂ was added dropwise to a suspension of above sodium sulfonate 106-2, 107-2, or 96-2 in toluene at 0-5° C. with stirring. After the addition was completed, DMF (1 drop) was added, then the resulting mixture was heated to 120° C. and stirred at reflux for 5 hours. The reaction mixture was concentrated to afford the corresponding crude sulfonyl chloride 106-3, 107-3, and 96-3, which were used directly in the next step. A solution of Intermediate L (1.0 equiv.), sulfonyl chloride 106-3, 107-3, or 96-3 (1.2 equiv.), and TEA (8.0 equiv.) in DCM was stirred at room temperature for 2 hours. The reaction mixture was washed with sat. aq. NH₄Cl. The organic layers were dried over Na₂SO₄, filtered, and concentrated. The residue was purified by flash column chromatography to afford 106-4, 107-4, and 96-4, respectively. 106-4 (12 mg, 55% yield): ¹H NMR (600 MHz, CDCl₃) δ 8.35 (d, J=2.2 Hz, 1H), 8.05 (dd, J=9.3, 2.3 Hz, 1H), 7.74 (d, J=8.6 Hz, 2H), 7.41-7.36 (m, 2H), 7.34-7.22 (m, 6H), 7.09 (d, J=8.5 Hz, 1H), 7.03-6.98 (m, 2H), 6.82-6.76 (m, 2H), 6.58 (d, J=9.4 Hz, 1H), 3.91 (d, J=9.0 Hz, 1H), 3.82-3.71 (m, 6H), 3.33-3.19 (m, 10H), 3.11 (dd, J=13.9, 5.0 Hz, 1H), 3.05 (dd, J=13.9, 6.9 Hz, 1H), 2.93-2.77 (m, 5H), 2.77-2.57 (m, 6H), 2.48-2.38 (m, 4H), 2.37-2.20 (m, 4H), 2.17-2.08 (m, 2H), 1.99-1.89 (m, 3H), 1.78 (dq, J=13.9, 6.8 Hz, 1H), 1.63-1.56 (m, 1H), 1.49-1.42 (m, 1H), 1.30 (t, J=7.2 Hz, 2H), 0.95 (s, 3H) ppm. LC/MS (ESI) m/z 1222.1; [M+H]⁺ calcd for C₅₆H₇₂ClF₃N₇O₁₀S₄⁺: 1222.39. 107-4 (23 mg, 41% yield): ¹H NMR (600 MHz, CDCl₃) δ 8.33 (d, J=2.2 Hz, 1H), 8.02 (dd, J=9.1, 2.3 Hz, 1H), 7.78 (d, J=8.7 Hz, 2H), 7.40-7.35 (m, 2H), 7.32-7.28 (m, 4H), 7.27-7.22 (m, 1H), 7.01 (dd, J=9.1, 7.2 Hz, 3H), 6.78 (d, J=8.7 Hz, 2H), 6.56 (d, J=9.3 Hz, 1H), 3.90 (s, 1H), 3.81-3.71 (m, 4H), 3.70 (s, 3H), 3.28 (dd, J=11.7, 6.7 Hz, 8H), 3.10 (dd, J=13.9, 4.9 Hz, 1H), 3.04 (dd, J=14.0, 6.7 Hz, 1H), 2.96-2.82 (m, 6H), 2.81-2.67 (m, 3H), 2.63 (t, J=5.3 Hz, 4H), 2.46 (s, 4H), 2.38 (t, J=7.2 Hz, 2H), 2.33-2.18 (m, 4H), 2.18-2.10 (m, 2H), 2.02-1.92 (m, 3H), 1.90-1.83 (m, 2H), 1.82-1.73 (m, 3H), 1.63-1.54 (m, 1H), 1.49-1.42 (m, 1H), 0.94 (s, 3H) ppm. LC/MS (ESI) m/z 1250.3; [M+H]⁺ calcd for C₅₈H₇₆ClF₃N₇O₁₀S₄⁺: 1250.42. 96-4 (12 mg, 52% yield): LC/MS (ESI) m/z 1278.3; [M+H]⁺ calcd for C₆₀H₈₀ClF₃N₇O₁₀S₄⁺: 1278.45.

Preparation of 3-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)propanoic acid (106-5), 5-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)pentanoic acid (107-5), and 7-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)heptanoic acid (96-5): A solution of 106-4, 107-4, or 96-4 (1.0 equiv.) and LiOH·H₂O (2.0 equiv.) in THF/MeOH/H₂O was stirred at room temperature for 2 hours. The reaction mixture was concentrated under vacuum to remove organic solvents, then adjusted to pH=4-5 with 5% aq. Citric acid and extracted with EtOAc. The combined organic layers were washed with brine, dried over Na₂SO₄, filtered, and concentrated to afford the corresponding crude carboxylic acids 106-5, 107-5, and 96-5, which were used directly in the next step.

106-5 (11.1 mg, 92% yield): LC/MS (ESI) m/z 1208.3; [M+H]⁺ calcd for C₅₅H₇₀ClF₃N₇O₁₀S₄⁺: 1208.37.

107-5 (19.5 mg, 86% yield): LC/MS (ESI) m/z 1236.3; [M+H]⁺ calcd for C₅₇H₇₄ClF₃N₇O₁₀S₄⁺: 1236.40

96-5 (9.7 mg, 82% yield): LC/MS (ESI) m/z 1264.5; [M+H]⁺ calcd for C₅₉H₇₈ClF₃N₇O₁₀S₄⁺: 1264.43.

Preparation of (2S,4R)-1-((S)-2-(3-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)propanamido)-3,3-dimethylbutanoyl)-4-hydroxy-N—((S)-1-(4-(4-methylthiazol-5-yl)phenyl)ethyl)pyrrolidine-2-carboxamide (compound #106), (2S,4R)-1-((S)-2-(5-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)pentanamido)-3,3-dimethylbutanoyl)-4-hydroxy-N—((S)-1-(4-(4-methylthiazol-5-yl)phenyl)ethyl)pyrrolidine-2-carboxamide (compound #107), and (2S,4R)-1-((S)-2-(7-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)heptanamido)-3,3-dimethylbutanoyl)-4-hydroxy-N—((S)-1-(4-(4-methylthiazol-5-yl)phenyl)ethyl)pyrrolidine-2-carboxamide (compound #96): A solution of VHL-L (1.0 equiv.) and DIPEA (5.0 equiv.) in DMF was added to a solution of 106-5, 107-5, or 96-5 (1.0 equiv.) and HATU (1.2 equiv.) in DMF. The resulting mixture was stirred at room temperature for 2 hours, then diluted with DCM and washed subsequently with water and sat. aq. NH₄Cl solution. The organic layer was dried over Na₂SO₄, filtered, and concentrated. The residue was purified by preparative TLC (DCM/MeOH) to afford the desired compound.

Compound #106 (2.7 mg, 37% yield): ¹H NMR (600 MHz, CDCl₃) δ 8.69 (s, 1H), 8.33 (d, J=2.3 Hz, 1H), 8.11 (dd, J=9.4, 2.2 Hz, 1H), 7.69 (d, J=8.7 Hz, 2H), 7.43-7.36 (m, 7H), 7.31 (s, 4H), 7.01-6.97 (m, 2H), 6.91 (d, J=8.7 Hz, 1H), 6.78 (d, J=8.8 Hz, 2H), 6.61 (d, J=9.3 Hz, 1H), 5.13-5.06 (m, 1H), 4.68 (t, J=8.0 Hz, 1H), 4.57 (d, J=8.8 Hz, 1H), 4.49 (s, 1H), 4.00 (d, J=11.4 Hz, 1H), 3.91 (s, 1H), 3.77 (t, J=6.1 Hz, 2H), 3.74-3.64 (m, 2H), 3.64-3.58 (m, 1H), 3.44 (p, J=1.6 Hz, 1H), 3.30-3.16 (m, 9H), 3.11 (dd, J=13.9, 5.2 Hz, 1H), 3.05 (dd, J=14.0, 7.0 Hz, 1H), 2.83 (s, 2H), 2.81-2.56 (m, 8H), 2.52 (s, 3H), 2.47-2.18 (m, 14H), 2.18-1.98 (m, 4H), 1.98-1.80 (m, 3H), 1.68-1.36 (m, 6H), 1.05 (s, 9H), 0.94 (s, 3H) ppm. LC/MS (ESI) m/z 1634.7; [M+H]⁺ calcd for C₇₈H₁₀₀ClF₃N₁₁O₁₂S₅⁺: 1634.58.

Compound #107 (3.4 mg, 43% yield): ¹H NMR (600 MHz, CDCl₃) δ 8.69 (s, 1H), 8.33 (d, J=2.3 Hz, 1H), 8.09 (dd, J=9.1, 2.2 Hz, 1H), 7.74 (d, J=9.0 Hz, 2H), 7.44-7.35 (m, 7H), 7.34-7.22 (m, 4H), 7.01-6.97 (m, 2H), 6.77 (d, J=8.7 Hz, 2H), 6.59 (dd, J=15.3, 9.1 Hz, 2H), 5.10 (p, J=7.1 Hz, 1H), 4.71 (t, J=8.1 Hz, 1H), 4.63 (d, J=9.1 Hz, 1H), 4.50 (s, 1H), 4.07 (d, J=11.4 Hz, 1H), 3.90 (d, J=8.7 Hz, 1H), 3.77 (t, J=6.1 Hz, 2H), 3.70 (pt, J=9.5, 4.6 Hz, 2H), 3.61 (dd, J=11.5, 3.5 Hz, 1H), 3.30-3.17 (m, 8H), 3.11 (dd, J=13.9, 5.1 Hz, 1H), 3.05 (dd, J=13.9, 6.9 Hz, 1H), 2.94-2.81 (m, 4H), 2.79-2.55 (m, 8H), 2.52 (s, 3H), 2.47-2.35 (m, 6H), 2.35-2.15 (m, 10H), 2.15-2.05 (m, 3H), 1.97-1.86 (m, 3H), 1.81 (p, J=7.8 Hz, 1H), 1.77-1.69 (m, 2H), 1.66-1.53 (m, 2H), 1.49 (d, J=7.0 Hz, 3H), 1.46-1.39 (m, 1H), 1.05 (s, 9H), 0.94 (s, 3H) ppm. LC/MS (ESI) m/z 1662.7; [M+H]⁺ calcd for C₈₀H₁₀₄ClF₃N₁₁O₁₂S₅⁺: 1662.61.

Compound #96 (3.8 mg, 32% yield). ¹H NMR (600 MHz, CDCl₃) δ 8.68 (s, 1H), 8.32 (d, J=2.3 Hz, 1H), 8.09 (dd, J=9.3, 2.3 Hz, 1H), 7.74 (d, J=8.7 Hz, 2H), 7.42-7.35 (m, 7H), 7.33-7.21 (m, 4H), 7.01-6.96 (m, 2H), 6.77 (d, J=9.0 Hz, 2H), 6.60 (d, J=9.4 Hz, 1H), 6.54 (d, J=9.1 Hz, 1H), 5.08 (p, J=7.1 Hz, 1H), 4.67 (t, J=8.2 Hz, 1H), 4.62 (d, J=9.2 Hz, 1H), 4.48 (s, 1H), 4.06 (d, J=11.4 Hz, 1H), 3.90 (s, 1H), 3.76 (t, J=6.1 Hz, 2H), 3.73-3.64 (m, 2H), 3.59 (dd, J=11.5, 3.4 Hz, 1H), 3.25 (q, J=6.2 Hz, 8H), 3.10 (dd, J=13.9, 5.1 Hz, 1H), 3.05 (dd, J=13.9, 6.9 Hz, 1H), 2.93-2.87 (m, 2H), 2.84 (s, 2H), 2.79-2.55 (m, 8H), 2.52 (s, 3H), 2.44-2.03 (m, 14H), 1.95-1.86 (m, 3H), 1.83-1.67 (m, 5H), 1.66-1.52 (m, 4H), 1.49 (d, J=6.9 Hz, 3H), 1.45-1.36 (m, 2H), 1.35-1.27 (m, 4H), 1.04 (s, 9H), 0.94 (s, 3H) ppm. LC/MS (ESI) m/z 1690.7; [M+H]⁺ calcd for C₈₂H₁₀₈ClF₃N₁₁O₁₂S₅⁺: 1690.64.

### Example 54: Preparation of Compound #100

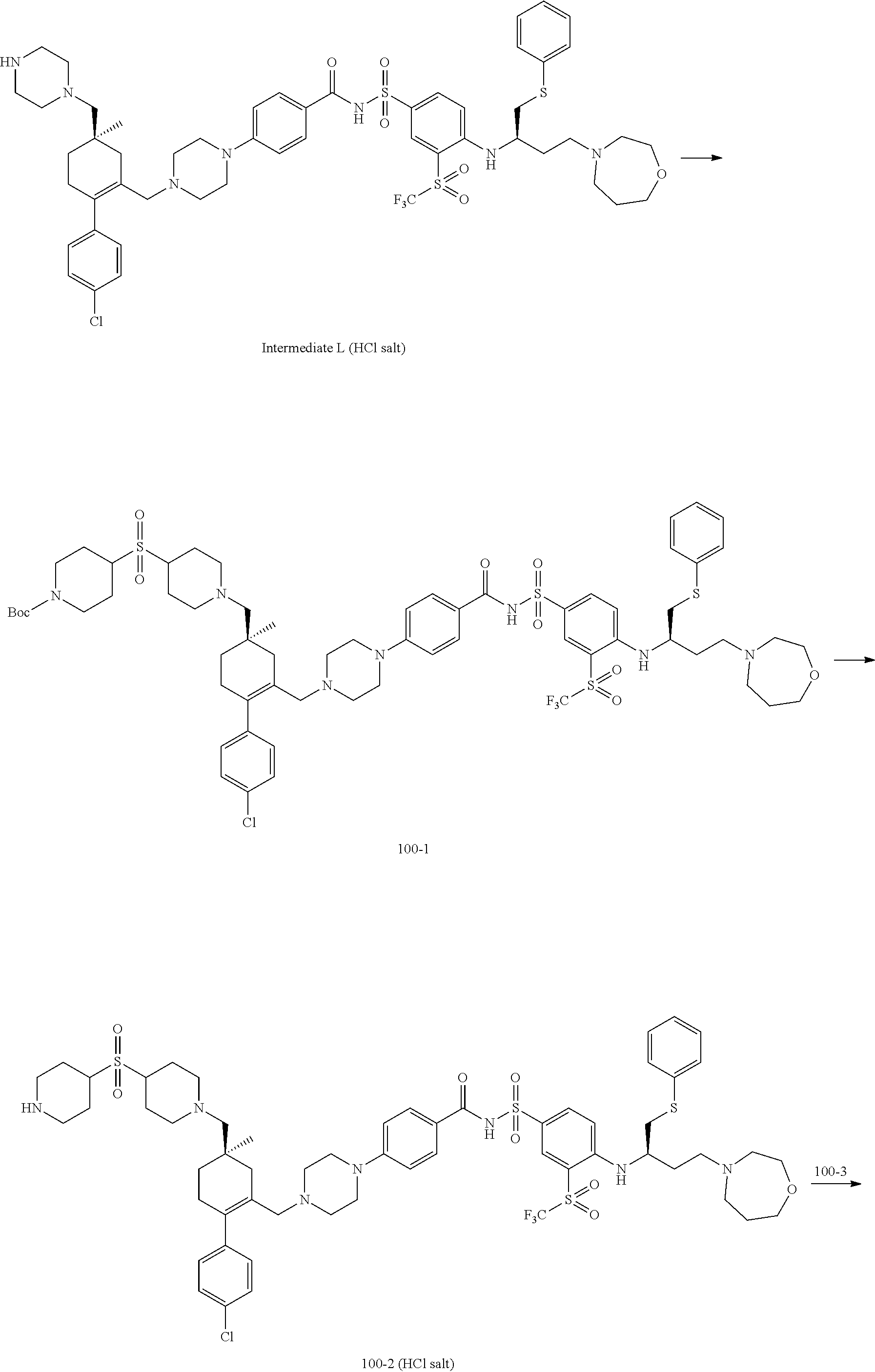 [SMILES: C[C@@]1(CN2CCNCC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1 ; CC(C)(C)OC(=O)N1CCC(S(=O)(=O)C2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)CC1 ; C[C@@]1(CN2CCC(S(=O)(=O)C3CCNCC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

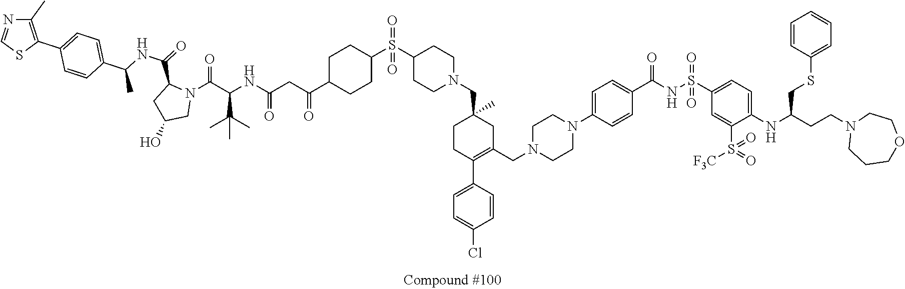 [SMILES: Cc1ncsc1-c1ccc([C@H](C)NC(=O)[C@@H]2C[C@@H](O)CN2C(=O)[C@@H](NC(=O)CC(=O)C2CCC(S(=O)(=O)C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1]

Preparation of tert-butyl 4-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)piperidine-1-carboxylate (100-1): A solution of Intermediate L (60 mg, 0.054 mmol), tert-butyl 4-(chlorosulfonyl)piperidine-1-carboxylate (36.8 mg, 0.13 mmol), and DIPEA (150 μL, 0.866 mmol) in DCM (1 mL) was stirred at room temperature for 3 days. The reaction mixture was concentrated, the residue was purified by flash column chromatography (0% to 5% of MeOH in DCM) to afford 100-1 (57 mg, 80% yield). ¹H NMR (600 MHz, Chloroform-d) δ 8.36 (d, J=2.2 Hz, 1H), 8.07 (dd, J=9.2, 2.2 Hz, 1H), 7.74 (d, J=8.6 Hz, 2H), 7.40-7.37 (m, 2H), 7.37-7.23 (m, 5H), 7.11 (d, J=8.3 Hz, 1H), 7.02-6.97 (m, 2H), 6.79 (d, J=8.6 Hz, 2H), 6.59 (d, J=9.3 Hz, 1H), 4.36-4.17 (m, 3H), 3.92 (d, J=7.6 Hz, 1H), 3.81-3.68 (m, 4H), 3.37 (t, J=4.9 Hz, 4H), 3.28 (t, J=5.1 Hz, 4H), 3.11 (dd, J=13.8, 5.1 Hz, 1H), 3.08-2.99 (m, 2H), 2.94-2.57 (m, 14H), 2.42 (s, 4H), 2.35-2.21 (m, 4H), 2.17-2.01 (m, 4H), 1.95 (d, J=14.8 2 Hz, 3H), 1.81-1.65 (m, 3H), 1.64-1.54 (m, 1H), 1.49 (s, 9H), 0.95 (s, 3H) ppm. LC/MS (ESI) m/z 1319.6; [M+H]⁺ calcd for C₆₂H₈₃ClF₃N₈O₁₀S₄⁺: 1319.48.

Preparation of N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-((4-(piperidin-4-ylsulfonyl)piperazin-1-yl)methyl)-3,4,5,6-tetrahydro-[1,1′-biphenyl]-2-yl)methyl)piperazin-1-yl)benzamide hydrochloride (100-2): To solution of 100-1 (30 mg, 0.023 mmol) in DCM (0.5 mL) was added HCl dioxane solution (4N, 0.5 mL), then stirred at room temperature for 2 hours. The reaction mixture was concentrated to afford crude 100-2 (28 mg, quantitative yield) as an HCl salt, which was used directly in the next step. LC/MS (ESI) m/z 1219.6; [M+H]⁺ calcd for C₅₇H₇₅ClF₃N₈O₈S₄⁺: 1219.42.

Preparation of (2S,4R)-1-((S)-2-(3-(4-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)sulfonyl)piperidin-1-yl)-3-oxopropanamido)-3,3-dimethylbutanoyl)-4-hydroxy-N—((S)-1-(4-(4-methylthiazol-5-yl)phenyl)ethyl)pyrrolidine-2-carboxamide (compound #100): General Procedure H was applied to afford compound #100 (4.4 mg, 23% yield in two steps). ¹H NMR (600 MHz, Chloroform-d) δ 8.69 (d, J=3.2 Hz, 1H), 8.35 (dd, J=7.4, 2.3 Hz, 1H), 8.14 (q, J=11.6, 10.1 Hz, 2H), 7.76 (d, J=8.6 Hz, 2H), 7.60-7.52 (m, 1H), 7.44-7.35 (m, 6H), 7.34-7.25 (m, 5H), 7.17 (s, 1H), 7.00 (dd, J=8.4, 2.6 Hz, 2H), 6.78 (dd, J=15.8, 8.7 Hz, 2H), 6.62 (d, J=9.3 Hz, 1H), 5.13-5.04 (m, 1H), 4.84-4.59 (m, 2H), 4.59-4.44 (m, 2H), 4.21-3.86 (m, 3H), 3.77 (t, J=6.0 Hz, 2H), 3.75-3.65 (m, 2H), 3.60 (dd, J=11.4, 3.6 Hz, 1H), 3.47-3.28 (m, 6H), 3.26-3.02 (m, 8H), 2.85 (s, 2H), 2.79-2.19 (m, 20H), 2.17-1.98 (m, 3H), 1.95-1.57 (m, 9H), 1.53-1.39 (m, 4H), 1.28 (s, 5H), 1.12 (d, J=4.1 Hz, 9H), 0.93 (s, 3H) ppm. LC/MS (ESI) m/z 1732.0; [M+H]⁺ calcd for Cs₃H₁₀₇ClF₃N₁₂O₁₃S₅⁺: 1731.63.

### Example 55: Preparation of Compound #105

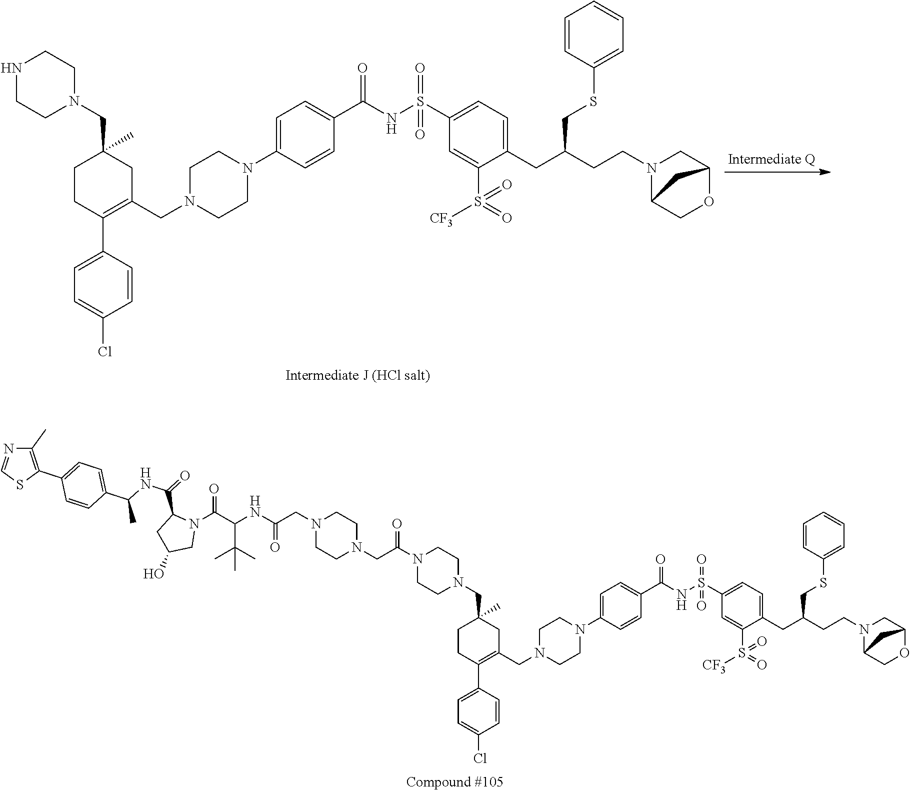 [SMILES: C[C@@]1(CN2CCNCC2)CCC(c2ccc(Cl)cc2)=C(CN2CCN(c3ccc(C(=O)NS(=O)(=O)c4ccc(C[C@H](CCN5C[C@H]6C[C@@H]5CO6)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)C(NC(=O)CN2CCN(CC(=O)N3CCN(C[C@]4(C)CCC(c5ccc(Cl)cc5)=C(CN5CCN(c6ccc(C(=O)NS(=O)(=O)c7ccc(C[C@H](CCN8C[C@H]9C[C@@H]8CO9)CSc8ccccc8)c(S(=O)(=O)C(F)(F)F)c7)cc6)CC5)C4)CC3)CC2)C(C)(C)C)cc1]

Compound #105 was prepared by following General Procedure H. 4-(4-(((4-(((R)-4-((1R,4R)-2-oxa-5-azabicyclo[2.2.1]heptan-5-yl)-1-(phenylthio)butan-2-yl)amino)-3-((trifluoromethyl)sulfonyl)phenyl)sulfonyl)carbamoyl)phenyl)-1-(((R)-4′-chloro-4-((4-(2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetyl)piperazin-1-yl)methyl)-4-methyl-3,4,5,6-tetrahydro-[1,1′-biphenyl]-2-yl)methyl)piperazin-1-ium (compound #105) (10.1 mg, 15% yield). ¹H NMR (600 MHz, Chloroform-d) δ 8.69 (s, 1H), 8.32 (d, J=2.5 Hz, 1H), 8.09 (dd, J=9.1, 2.2 Hz, 1H), 7.83 (d, J=8.1 Hz, 1H), 7.78 (d, J=8.5 Hz, 2H), 7.49 (d, J=7.0 Hz, 1H), 7.44-7.35 (m, 6H), 7.34-7.21 (m, 5H), 7.08 (d, J=8.3 Hz, 1H), 7.03-6.98 (m, 2H), 6.78 (d, J=8.7 Hz, 2H), 6.59 (d, J=9.4 Hz, 1H), 5.10 (p, J=7.0 Hz, 1H), 4.78 (t, J=8.0 Hz, 1H), 4.56-4.47 (m, 2H), 4.42 (t, J=2.0 Hz, 1H), 4.23 (d, J=11.5 Hz, 1H), 3.99 (d, J=8.1 Hz, 1H), 3.93 (d, J=7.9 Hz, 1H), 3.66-3.46 (m, 9H), 3.32-3.14 (m, 6H), 3.13-2.99 (m, 2H), 2.98-2.79 (m, 5H), 2.75 (dt, J=13.6, 7.1 Hz, 1H), 2.71-2.40 (m, 17H), 2.39-2.21 (m, 8H), 2.12-1.99 (m, 2H), 1.88 (t, J=11.8 Hz, 2H), 1.82-1.70 (m, 2H), 1.70-1.60 (m, 1H), 1.50 (d, J=6.9 Hz, 3H), 1.42 (dt, J=12.0, 5.6 Hz, 1H), 1.07 (s, 9H), 0.96 (s, 3H). LC/MS (ESI) m/z 1680.8; [M+H]⁺ calcd for Cs₃H₁₀₆ClF₃N₁₃O₁₁S₄⁺: 1680.67.

### Example 56: Preparation of Compound #108

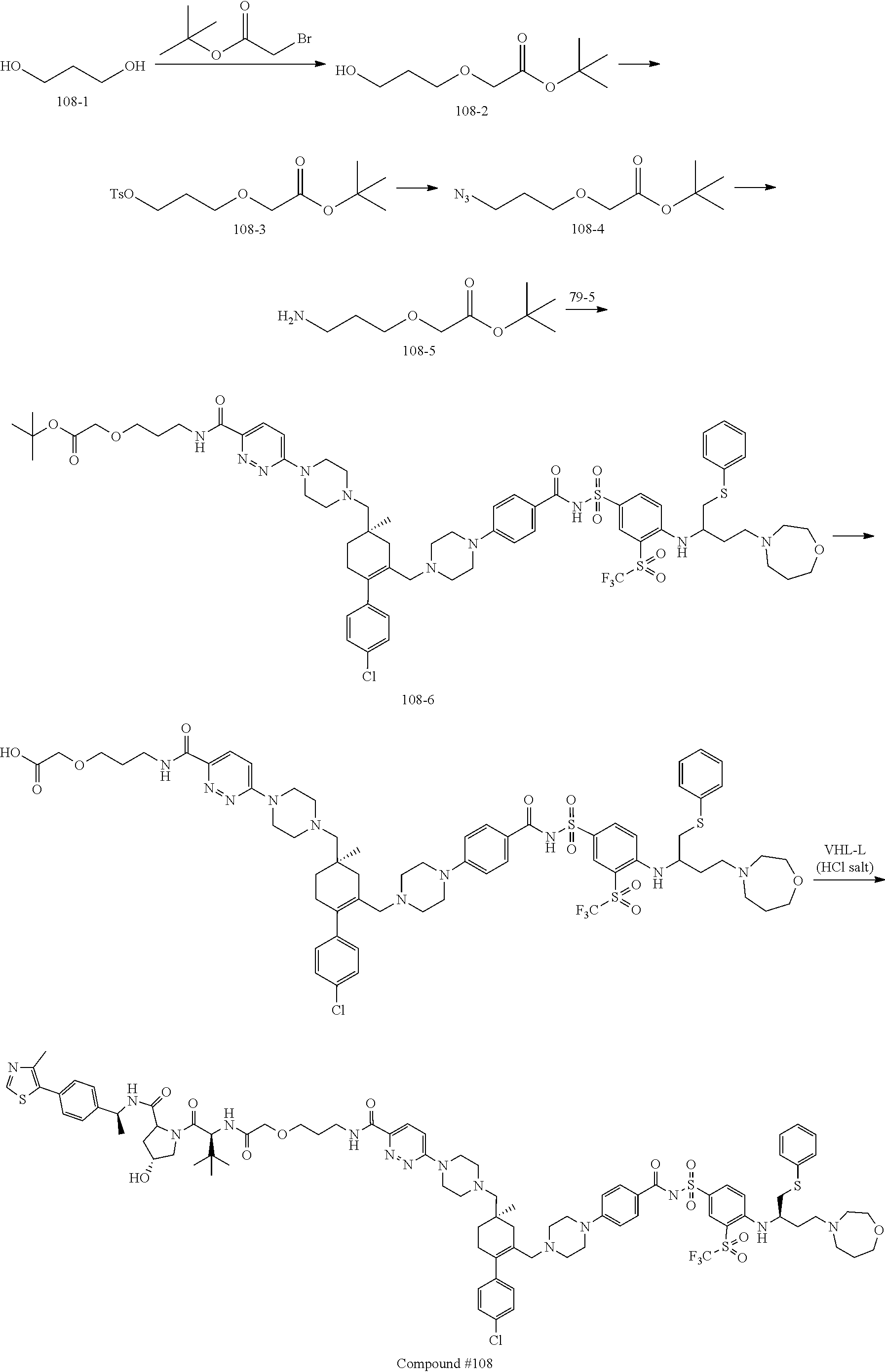

Preparation of tert-butyl 2-(3-hydroxypropoxy)acetate (108-2): Under argon atmosphere, to a suspension of 108-1 (3 g, 39.4 mmol) and 60% NaH (1.42 g, 35.5 mmol) in anhydrous THF (50 mL) was added tert-butyl 2-bromoacetate (4.08 mL, 27.6 mmol) dropwise at 0-5° C. with stirring. After addition was completed, the resulting mixture was warmed up to room temperature and stirred an additional 16 hours. The reaction was diluted with EtOAc and washed with sat. aq. NH₄Cl, dried over Na₂SO₄, filtered, and concentrated. The residue was purified by flash column chromatography (0% to 50% of EtOAc in hexanes) to afford 108-2 (1.57 g, 29% yield). ¹H NMR (600 MHz, Chloroform-d) δ 3.96 (s, 2H), 3.81 (q, J=5.8 Hz, 2H), 3.68 (t, 2H), 3.00-2.87 (br, 1H), 1.83 (p, J=5.7 Hz, 2H), 1.48 (s, 9H) ppm. Preparation of tert-butyl 2-(3-(tosyloxy)propoxy)acetate (108-3): A suspension of TsCl (750 mg, 3.93 mmol), 108-2 (500 mg, 2.63 mmol), and TEA (1.5 mL, 10.53 mmol) in DCM (5 mL) was stirred at room temperature overnight. The reaction mixture was washed with sat. aq. NH₄Cl solution. The organic layer was dried over Na₂SO₄, filtered, and concentrated. The residue was purified by flash column chromatography (0% to 60% of EtOAc in hexanes) to afford 108-3 (740 mg, 82% yield). ¹H NMR (600 MHz, CDCl₃) δ 7.84-7.79 (m, 2H), 7.36 (d, J=8.1 Hz, 2H), 4.19 (t, J=6.3 Hz, 2H), 3.88 (s, 2H), 3.57 (t, J=6.0 Hz, 2H), 2.47 (s, 3H), 1.98 (p, J=6.1 Hz, 2H), 1.49 (s, 9H) ppm. LC/MS (ESI) m/z 345.2; [M+H]⁺ calcd for C₁₆H₂₅O₆S⁺: 345.14.

Preparation of tert-butyl 2-(3-azidopropoxy)acetate (108-4): A suspension of 108-3 (740 mg, 2.15 mmol) and NaN₃ (1.1 g, 17.21 mmol) in DMF (5 mL) was stirred at 70° C. overnight. The reaction mixture was cooled to room temperature and poured into water, then extracted with EtOAc. The combined organic layers were washed with sat. aq. NH₄Cl, dried over Na₂SO₄, filtered, and concentrated. The residue was purified by flash column chromatography (0% to 50% of EtOAc in hexanes) to afford 108-4 (420 mg, 91% yield). ¹H NMR (600 MHz, CDCl₃) δ 3.98 (s, 2H), 3.62 (t, J=6.0 Hz, 2H), 3.47 (t, J=6.7 Hz, 2H), 1.94-1.86 (m, 2H), 1.51 (s, 9H) ppm. LC/MS (ESI) m/z 216.3; [M+H]⁺ calcd for C₉H₁₈N₃O₃⁺: 216.13.

Preparation of tert-butyl 2-(3-aminopropoxy)acetate (108-5): A suspension of 108-4 (420 mg, 1.95 mmol) and 10% Pd/C (105 mg) in MeOH (5 mL) was hydrogenated with a hydrogen balloon overnight. The reaction mixture was filtered through celite, the filtrate was concentrated to afford crude 108-5 (369 mg, quantitative yield), which was used directly in the next step. ¹H NMR (600 MHz, DMSO) δ 3.93 (d, J=1.3 Hz, 2H), 3.47 (td, J=6.3, 4.2 Hz, 2H), 2.59 (t, J=6.7 Hz, 1H), 2.53 (t, 1H), 1.62 (p, J=6.7 Hz, 1H), 1.56 (p, J=6.6 Hz, 1H), 1.43 (d, J=1.1 Hz, 9H) ppm. LC/MS (ESI) m/z 190.2; [M+H]⁺ calcd for C₉H₂₀NO₃⁺: 190.14.

Preparation of tert-butyl 2-(3-(6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyridazine-3-carboxamido)propoxy)acetate (108-6): A solution of 108-5 (2.5 mg, 0.013 mmol), 79-5 (9 mg, 0.008 mmol), EDC (5 mg, 0.026 mmol), and DMAP (3.2 mg, 0.026 mmol) in DCM (0.3 mL) was stirred at room temperature overnight. The reaction mixture was washed with water and sat. aq. NH₄Cl, dried over Na₂SO₄, filtered, and concentrated. The residue was purified by preparative TLC (DCM/MeOH=10/1) to afford 108-6 (8.1 mg, 78% yield). ¹H NMR (600 MHz, CDCl₃) δ 8.35 (d, J=2.2 Hz, 1H), 8.12 (t, J=6.1 Hz, 1H), 8.07 (dd, J=9.3, 2.3 Hz, 1H), 7.98 (d, J=9.5 Hz, 1H), 7.74 (d, J=8.8 Hz, 2H), 7.41-7.37 (m, 2H), 7.34-7.23 (m, 6H), 7.10 (d, J=8.4 Hz, 1H), 7.04-6.99 (m, 2H), 6.93 (d, J=9.6 Hz, 1H), 6.81-6.76 (m, 2H), 6.58 (d, J=9.3 Hz, 1H), 3.99 (s, 2H), 3.94-3.87 (m, 1H), 3.81-3.70 (m, 8H), 3.64 (dt, J=15.2, 6.4 Hz, 4H), 3.29 (t, J=5.2 Hz, 4H), 3.11 (dd, J=13.9, 5.1 Hz, 1H), 3.05 (dd, J=14.0, 6.9 Hz, 1H), 2.90 (s, 2H), 2.85-2.59 (m, 10H), 2.43 (s, 4H), 2.38-2.16 (m, 6H), 2.16-2.08 (m, 1H), 1.95 (p, J=6.2 Hz, 4H), 1.77 (dt, J=13.9, 6.9 Hz, 1H), 1.66 (tt, J=8.8, 5.4 Hz, 2H), 1.50 (s, 9H), 1.00 (s, 3H) ppm. LC/MS (ESI) m/z 1365.7; [M+H]⁺ calcd for C₆₆H₈₅ClF₃N₁₀O₁₀S₃⁺: 1365.52.

Preparation of 2-(3-(6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)pyridazine-3-carboxamido)propoxy)acetic acid (108-7): To a solution of 108-6 (8 mg, 0.0059 mmol) in DCM (0.2 mL) was added TFA (0.05 mL), then stirring at room temperature for 2 hours. The reaction mixture was concentrated to afford 108-7 (8 mg, quantitative yield), which was used directly in the next step. LC/MS (ESI) m/z 1309.6; [M+H]⁺ calcd for C₆₂H₇₇ClF₃N₁₀O₁₀S₃⁺: 1309.46.

Preparation of 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)propyl)pyridazine-3-carboxamide (compound #108): A solution of VHL-L (3.6 mg, 0.007 mmol, HCl salt) and TEA (20 μL, 0.14 mmol) in DCM (0.2 mL) was added to a solution of 108-7 (8 mg, 0.006 mmol) and HATU (2.9 mg, 0.0076 mmol) in DCM (0.2 mL). The resulting mixture was stirred at room temperature for 2 hours, then washed with sat. aq. NH₄Cl. The organic layers were dried over Na₂SO₄, filtered, and concentrated. The residue was purified by preparative TLC (DCM/MeOH=10/1) to afford compound #108 (3.7 mg, 36% yield). ¹H NMR (600 MHz, CDCl₃) δ 8.67 (d, J=11.9 Hz, 2H), 8.33 (d, J=2.2 Hz, 1H), 8.09 (dd, J=9.3, 2.3 Hz, 1H), 7.96 (d, J=9.5 Hz, 1H), 7.93 (d, J=7.7 Hz, 1H), 7.71 (d, J=8.9 Hz, 2H), 7.49 (d, J=9.7 Hz, 1H), 7.40-7.36 (m, 2H), 7.35-7.22 (m, 8H), 7.02-6.98 (m, 2H), 6.93 (d, J=9.5 Hz, 1H), 6.76 (d, J=9.0 Hz, 2H), 6.59 (d, J=9.4 Hz, 1H), 5.07 (p, J=7.0 Hz, 1H), 4.95 (t, J=8.1 Hz, 1H), 4.62 (d, J=9.7 Hz, 1H), 4.50 (s, 1H), 4.16 (d, J=15.9 Hz, 1H), 3.98 (d, J=11.4 Hz, 1H), 3.90 (s, 1H), 3.82-3.74 (m, 4H), 3.75-3.56 (m, 10H), 3.55-3.49 (m, 1H), 3.25 (t, J=5.0 Hz, 4H), 3.10 (dd, J=13.9, 5.1 Hz, 1H), 3.05 (dd, J=13.9, 7.0 Hz, 1H), 2.87 (s, 2H), 2.81-2.55 (m, 10H), 2.50 (s, 3H), 2.42-2.16 (m, 12H), 2.16-2.05 (m, 3H), 2.01-1.87 (m, 4H), 1.79-1.54 (m, 2H), 1.50-1.43 (m, 1H), 1.39 (d, J=6.9 Hz, 3H), 1.08 (s, 9H), 0.97 (s, 3H) ppm. LC/MS (ESI) m/z 1735.7; [M+H]⁺ calcd for C₈₅H₁₀₇ClF₃N₁₄O₁₂S₄⁺: 1735.67.

### Example 57: Preparation of Compound #109

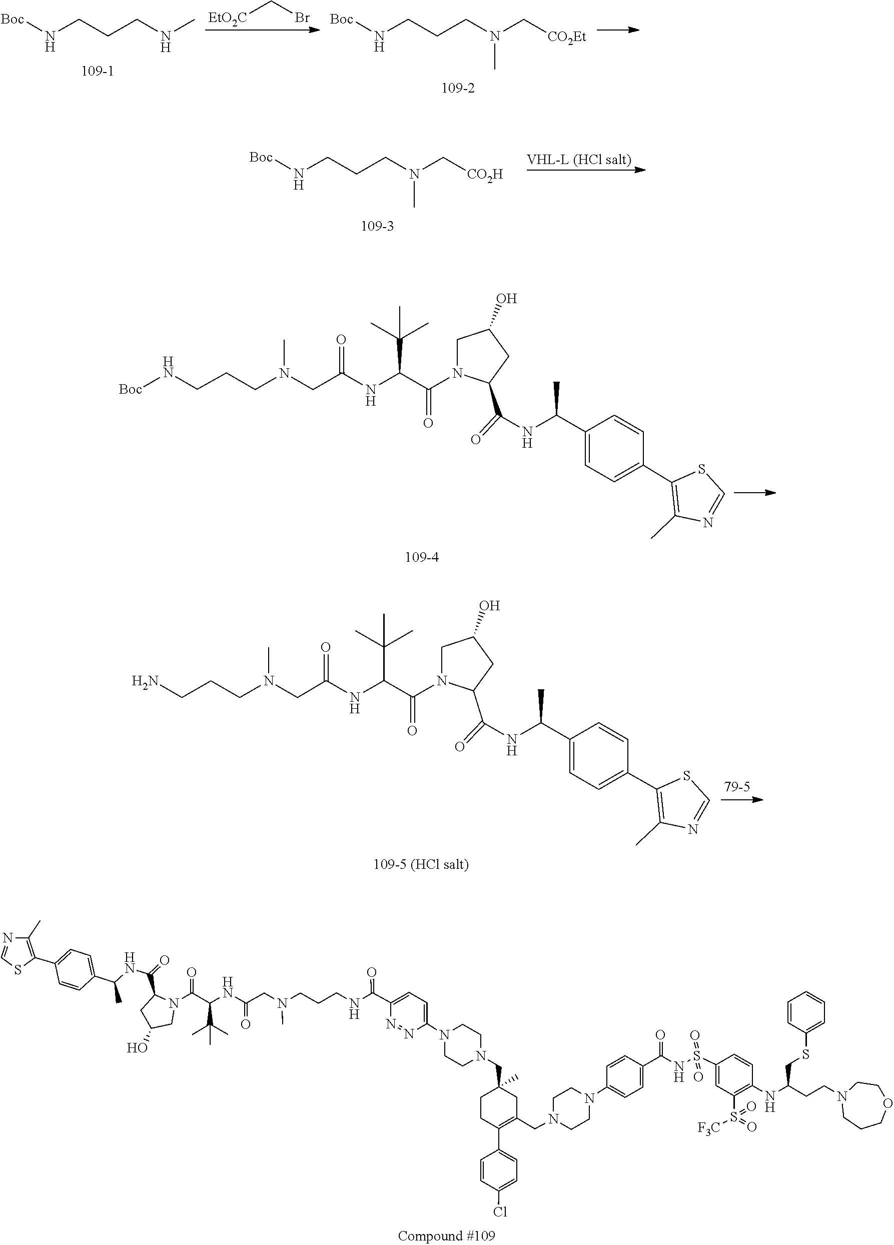

Compound #109 was prepared by following General Procedure A. ethyl N-(3-((tert-butoxycarbonyl)amino)propyl)-N-methylglycinate (109-2) (630 mg, 86% yield), ¹H NMR (600 MHz, Chloroform-d) δ 5.24 (s, 1H), 4.19 (q, J=7.1 Hz, 2H), 3.26-3.15 (m, 4H), 2.51 (t, J=6.7 Hz, 2H), 2.33 (s, 3H), 1.64 (p, J=7.6, 7.1 Hz, 2H), 1.44 (s, 9H), 1.28 (t, J=7.1 Hz, 3H) ppm. LC/MS (ESI) m/z 275.3; [M+H]⁺ calcd for C₁₃H₂₇N₂O₄⁺: 275.20. N-(3-((tert-butoxycarbonyl)amino)propyl)-N-methylglycine (109-3) (83 mg, 88% yield), LC/MS (ESI) m/z 247.0; [M+H]⁺ calcd for C₁₁H₂₃N₂O₄⁺: 247.17.

tert-butyl (3-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)amino)propyl)carbamate (109-4) (154 mg, 72% yield), LC/MS (ESI) m/z 673.5; [M+H]⁺ calcd for C₃₄H₅₃N₆O₆S⁺: 673.

(2S,4R)-1-((S)-2-(2-((3-aminopropyl)(methyl)amino)acetamido)-3,3-dimethylbutanoyl)-4-hydroxy-N—((S)-1-(4-(4-methylthiazol-5-yl)phenyl)ethyl)pyrrolidine-2-carboxamide hydrochloride (109-5): (5 mg, quantitative yield), LC/MS (ESI) m/z 573.5; [M+H]⁺ calcd for 2 C₂₉H₄₅N₆O₄S⁺: 573.32.

6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-N-(3-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)amino)propyl)pyridazine-3-carboxamide (compound #109) (6.9 mg, 52% yield). ¹H NMR (600 MHz, CDCl₃) δ 8.67 (s, 1H), 8.34 (d, J=2.3 Hz, 1H), 8.10 (dd, J=9.2, 2.3 Hz, 1H), 8.04 (d, J=7.7 Hz, 1H), 7.95 (d, J=9.5 Hz, 1H), 7.71 (d, J=8.7 Hz, 2H), 7.38 (dt, J=6.5, 1.2 Hz, 2H), 7.35-7.22 (m, 8H), 7.03-6.97 (m, 2H), 6.91 (d, J=9.5 Hz, 1H), 6.76 (d, J=9.0 Hz, 2H), 6.60 (d, J=9.4 Hz, 1H), 5.08 (p, J=7.1 Hz, 1H), 4.97 (t, J=8.1 Hz, 1H), 4.59-4.55 (m, 1H), 4.48 (s, 1H), 4.04 (d, J=11.3 Hz, 1H), 3.91 (s, 1H), 3.82-3.54 (m, 11H), 3.51 (dt, J=13.6, 6.8 Hz, 1H), 3.25 (t, J=5.2 Hz, 4H), 3.15-3.02 (m, 3H), 2.99 (d, J=16.9 Hz, 1H), 2.87 (s, 2H), 2.80-2.55 (m, 11H), 2.51 (s, 3H), 2.40 (d, J=7.2 Hz, 9H), 2.35-2.18 (m, 7H), 2.17-2.03 (m, 2H), 1.98-1.85 (m, 3H), 1.79-1.70 (m, 2H), 1.63 (q, J=7.4 Hz, 2H), 1.49-1.43 (m, 1H), 1.39 (d, J=6.9 Hz, 3H), 1.09 (s, 9H), 0.97 (s, 3H) ppm. LC/MS (ESI) m/z 1748.9; [M+H]⁺ calcd for C₈₆H₁₁₀ClF₃N₁₅O₁₁S₄⁺: 1748.70.

### Example 58: Preparation of Compound #110

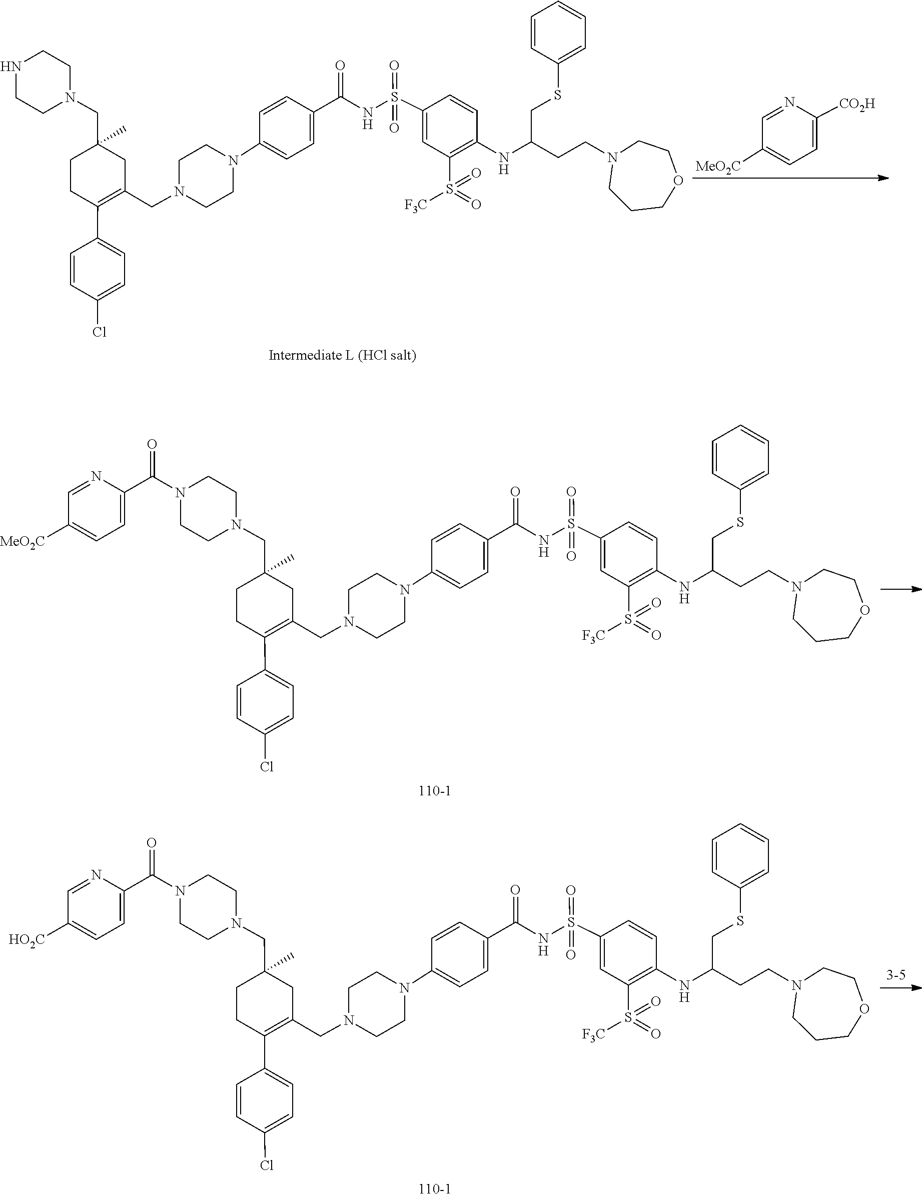 [SMILES: C[C@@]1(CN2CCNCC2)CCC(c2ccc(Cl)cc2)=C(CN2CCN(c3ccc(C(=O)NS(=O)(=O)c4ccc(NC(CCN5CCCOCC5)CSc5ccccc5)c(S(=O)(=O)C(F)(F)F)c4)cc3)CC2)C1 ; COC(=O)c1ccc(C(=O)O)nc1 ; COC(=O)c1ccc(C(=O)N2CCN(C[C@]3(C)CCC(c4ccc(Cl)cc4)=C(CN4CCN(c5ccc(C(=O)NS(=O)(=O)c6ccc(NC(CCN7CCCOCC7)CSc7ccccc7)c(S(=O)(=O)C(F)(F)F)c6)cc5)CC4)C3)CC2)nc1 ; C[C@@]1(CN2CCN(C(=O)c3ccc(C(=O)O)cn3)CC2)CCC(c2ccc(Cl)cc2)=C(CN2CCN(c3ccc(C(=O)NS(=O)(=O)c4ccc(NC(CCN5CCCOCC5)CSc5ccccc5)c(S(=O)(=O)C(F)(F)F)c4)cc3)CC2)C1]

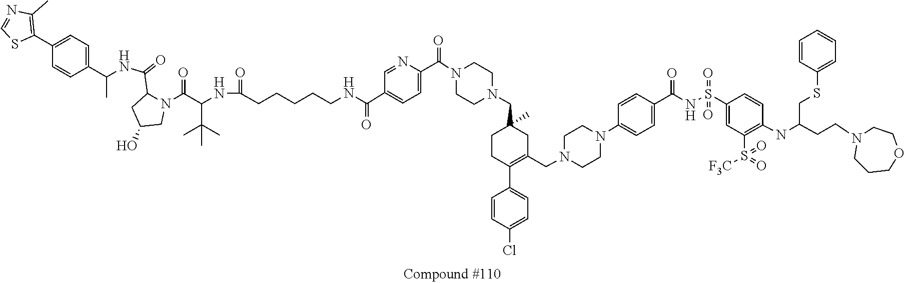

Preparation of methyl 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carbonyl)nicotinate (110-1): Intermediate L (20 mg, 0.018 mmol) was added to a mixture of 5-(methoxycarbonyl)picolinic acid (4.3 mg, 0.024 mmol), EDC (10.5 mg, 0.055 mmol), and DMAP (7 mg, 0.055 mmol) in DCM (1 mL), then the resulting mixture was stirred at room temperature overnight. The reaction mixture was washed with water and sat. aq. NH₄Cl, dried over Na₂SO₄, filtered, and concentrated. The residue was purified by preparative TLC (DCM/MeOH=10/1) to afford 110-1 (13 mg, 58% yield). ¹H NMR (600 MHz, CDCl₃) δ 9.18 (dd, J=2.1, 0.9 Hz, 1H), 8.40 (dd, J=8.1, 2.1 Hz, 1H), 8.35 (d, J=2.2 Hz, 1H), 8.06 (dd, J=9.3, 2.3 Hz, 1H), 7.77-7.68 (m, 3H), 7.41-7.35 (m, 2H), 7.34-7.22 (m, 5H), 7.09 (d, J=8.5 8 Hz, 1H), 7.03-6.97 (m, 2H), 6.80-6.75 (m, 2H), 6.59 (d, J=9.3 Hz, 1H), 4.00 (s, 3H), 3.92 (d, J=7.2 Hz, 1H), 3.86-3.71 (m, 6H), 3.53 (q, J=4.7 Hz, 2H), 3.27 (t, J=5.0 Hz, 4H), 3.11 (dd, J=13.9, 5.0 Hz, 1H), 3.05 (dd, J=13.9, 6.9 Hz, 1H), 2.93-2.62 (m, 10H), 2.63-2.53 (m, 2H), 2.42 (t, J=5.1 Hz, 4H), 2.36-2.07 (m, 5H), 2.01-1.91 (m, 3H), 1.78 (dq, J=13.8, 7.0 Hz, 1H), 1.66-1.58 (m, 1H), 1.48 (dt, J=11.9, 5.4 Hz, 1H), 0.97 (s, 3H) ppm. LC/MS (ESI) m/z 1235.5; [M+H]⁺ calcd for C₆₀H₇₁ClF₃N₈O₉S₃⁺: 1235.41.

Preparation of 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carbonyl)nicotinic acid (110-2): A solution of 110-1 (13 mg, 0.011 mmol) and LiOH·H₂O (1.3 mg, 0.032 mmol) in THF/MeOH/H₂O (0.1 mL/0.1 mL/0.1 mL) was stirred at room temperature for 2 hours. The reaction mixture was concentrated under vacuum to remove organic solvents, then adjusted to pH=4-5 with 10% aq. citric acid and extracted with EtOAc. The combined organic layers were washed with brine, dried over Na₂SO₄, filtered, and concentrated to afford crude 110-2 (12.8 mg, 79% yield), which was used directly in the next step. LC/MS (ESI) m/z 1221.5; [M+H]⁺ calcd for C₅₉H₆₉ClF₃N₈O₉S₃⁺: 1221.40.

Preparation of 6-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carbonyl)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)nicotinamide (compound #110): 3-5 (8.5 mg, 0.014 mmol) was added to a mixture of 110-2 (12 mg, 0.01 mmol), EDC (5.7 mg, 0.03 mmol), and DMAP (3.6 mg, 0.03 mmol) in DCM (1 mL), then the resulting mixture was stirred at room temperature overnight. The reaction mixture was washed with water and sat. aq. NH₄Cl, dried over Na₂SO₄, filtered, and concentrated. The residue was purified by preparative TLC (DCM/MeOH=10/1) to afford compound #110 (6.1 mg, 35% yield). ¹H NMR (600 MHz, CDCl₃) δ 9.03 (d, J=2.1 Hz, 1H), 8.68 (s, 1H), 8.33 (d, J=2.3 Hz, 1H), 8.30 (dd, J=8.0, 2.2 Hz, 1H), 8.12 (dd, J=9.3, 2.3 Hz, 1H), 7.86 (t, J=5.7 Hz, 1H), 7.70 (d, J=8.9 Hz, 2H), 7.61 (d, J=8.0 Hz, 1H), 7.42-7.33 (m, 6H), 7.33-7.21 (m, 6H), 7.01-6.96 (m, 2H), 6.76 (d, J=9.0 Hz, 2H), 6.61 (dd, J=9.4, 4.4 Hz, 2H), 5.07 (p, J=7.0 Hz, 1H), 4.63 (d, J=9.4 Hz, 1H), 4.58 (t, J=8.1 Hz, 1H), 4.48 (s, 1H), 3.96-3.81 (m, 3H), 3.81-3.64 (m, 4H), 3.64-3.54 (m, 2H), 3.53-3.39 (m, 3H), 3.22 (d, J=5.7 Hz, 4H), 3.10 (dd, J=13.9, 5.2 Hz, 1H), 3.05 (dd, J=13.9, 6.9 Hz, 1H), 2.82 (s, 2H), 2.77-2.54 (m, 6H), 2.54-2.47 (m, 4H), 2.42-2.18 (m, 20H), 2.11-2.03 (m, 2H), 1.92-1.83 (m, 3H), 1.80-1.55 (m, 4H), 1.51-1.39 (m, 5H), 1.03 (s, 9H), 0.97 (s, 3H) ppm. LC/MS (ESI) m/z 1760.5; [M+H]⁺ calcd for C₈₈H₁₁₀ClF₃N₁₃O₁₂S₄⁺: 1760.69.

### Example 59: Preparation of Compound #111

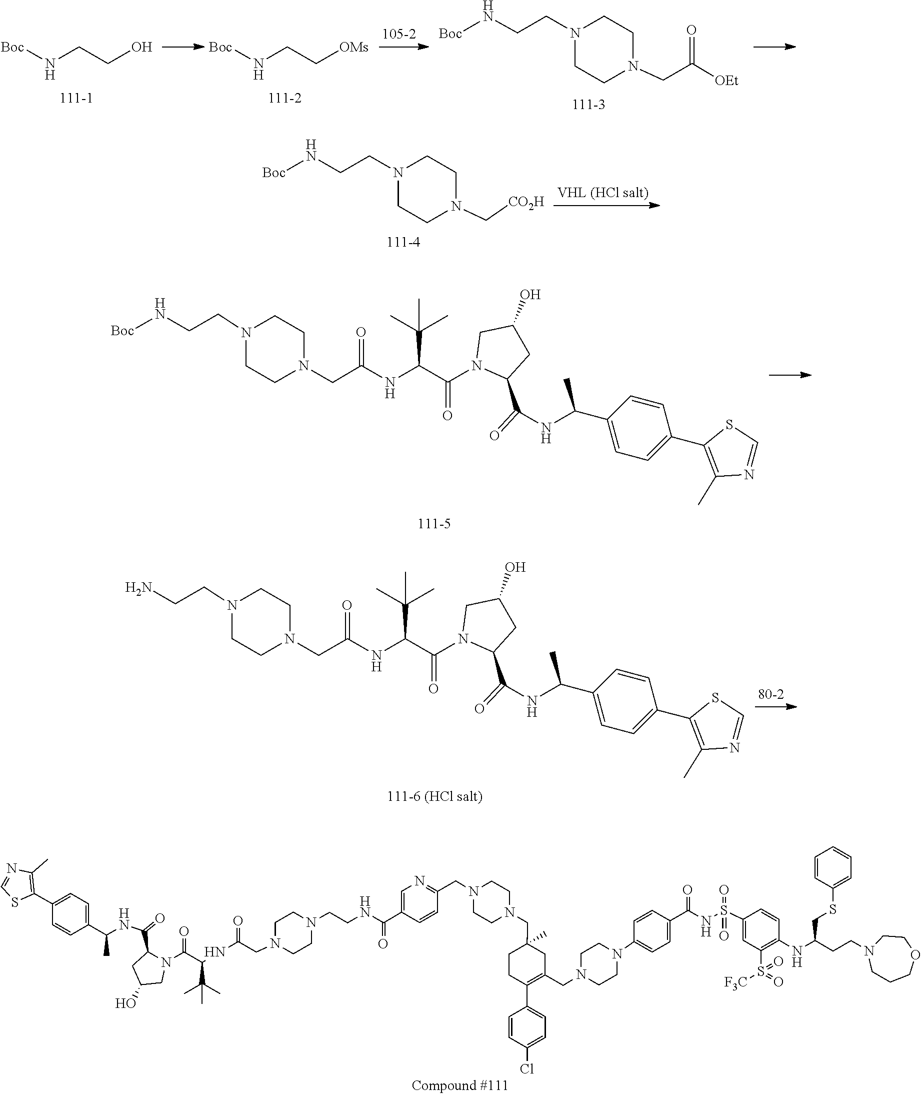

Preparation of 2-((tert-butoxycarbonyl)amino)ethyl methanesulfonate (111-2): MsCl (0.92 mL, 11.94 mmol) was added dropwise to a stirring solution of 111-1 (1.6 g, 1 mmol), and TEA (4 mL, 26.64 mmol) in DCM (20 mL) at 0-5° C. After the additional was completed, the resulting mixture was warmed up to room temperature and stirred for an additional 1 hour. The reaction mixture was washed with sat. aq. NH₄Cl. The organic layer was dried over Na₂SO₄, filtered, and concentrated. The residue was purified by flash column chromatography to afford 111-2 (2.18 g, 92% yield). ¹H NMR (600 MHz, CDCl₃) δ 4.92 (s, 1H), 4.31 (t, J=5.2 Hz, 2H), 3.50 (q, J=5.6 Hz, 2H), 3.06 (s, 3H), 1.47 (s, 9H).

Preparation of ethyl 2-(4-(2-((tert-butoxycarbonyl)amino)ethyl)piperazin-1-yl)acetate (111-3): A solution of 111-2 (0.7 g, 2.93 mmol), 105-2 (0.92 g, 4.39 mmol), and TEA (1.6 mL, 11.72 mmol) in acetonitrile (15 mL) was stirred at room temperature overnight. The reaction mixture was concentrated, and the residue was added DCM, then washed with sat. aq. NH₄Cl. The organic layer was dried over Na₂SO₄, filtered, and concentrated. The residue was purified by flash column chromatography to afford 111-3 (400 mg, 43% yield). ¹H NMR (600 MHz, CDCl₃) δ 4.98 (s, 1H), 4.21 (q, J=7.1 Hz, 2H), 3.29-3.19 (m, 4H), 2.63 (s, 4H), 2.58-2.43 (m, 6H), 1.47 (s, 9H), 1.30 (t, J=7.1 Hz, 3H) ppm. LC/MS (ESI) m/z 316.4; [M+H]⁺ calcd for C₁₅H₃₀N₃O₄⁺: 316.22.

Preparation of 2-(4-(2-((tert-butoxycarbonyl)amino)ethyl)piperazin-1-yl)acetic acid (111-4): To a solution of 111-3 (100 mg, 0.32 mmol) in MeOH/THF (2 mL/0.5 mL) was added a solution of LiOH·H₂O (65 mg, 1.55 mmol) in H₂O (0.5 mL), then stirred at room temperature overnight. The reaction mixture was adjusted to pH=5-6 with 1N aq. HCl, then concentrated. The residue was dried under vacuum to afford crude 111-4 (91 mg, quantitative yield), which was used directly in the next step. Preparation of tert-butyl (2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)ethyl)carbamate (111-5): A solution of VHL-L (140 mg, 0.27 mmol, HCl salt), and TEA (150 μL, 1.08 mmol) in DCM (2 mL) was added to a solution of 111-4 (91 22 mg, 0.32 mmol), and HATU (125 mg, 0.33 mmol) in DCM (2 mL). The resulting mixture was stirred at room temperature for 2 hours, then washed with sat. aq. NH₄Cl. The organic layers were dried over Na₂SO₄, filtered, and concentrated. The residue was purified by flash column chromatography (0% to 10% of MeOH in DCM) to afford 111-5 (120 mg, 62% yield in two steps). LC/MS (ESI) m/z 714.4; [M+H]⁺ calcd for C₃₆H₅₆N₇O₆S⁺: 714.40. Preparation of (2S,4R)-1-((S)-2-(2-(4-(2-aminoethyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide hydrochloride (111-6): To a solution of 111-5 (4.3 mg, 0.006 mmol) in DCM (0.2 mL) was added 4N HCl in dioxane (0.2 mL), then stirring at room temperature for 1 hour. The reaction mixture was concentrated to afford 111-6 (4.9 mg, quantitative yield) as an HCl salt, which was used directly in the next step. LC/MS (ESI) m/z 614.4; [M+H]⁺ calcd for C₃₁H₄₈N₇O₄S⁺: 614.35.

Preparation of 6-((4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)methyl)-N-(2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)ethyl)nicotinamide (compound #111): 111-6 (4.9 mg, 0.0075 mmol) was added to a mixture of 80-2 (6.6 mg, 0.0055 mmol), EDC (4.2 mg, 0.022 mmol), and DMAP (2.6 mg, 0.022 mmol) in DCM (0.3 mL). The resulting mixture was stirred at room temperature overnight, then washed with water and sat. aq. NH₄Cl, dried over Na₂SO₄, filtered, and concentrated. The residue was purified by preparative TLC (DCM/MeOH=10/1) to afford compound #111 (2.8 mg, 29% yield in two steps). ¹H NMR (600 MHz, CDCl₃) δ 8.92 (d, J=2.2 12 Hz, 1H), 8.68 (s, 1H), 8.33 (d, J=2.2 Hz, 1H), 8.12 (dd, J=8.1, 2.3 Hz, 1H), 7.97 (dd, J=9.1, 2.2 Hz, 1H), 7.94-7.89 (m, 2H), 7.51 (d, J=8.1 Hz, 1H), 7.46-7.35 (m, 7H), 7.32-7.21 (m, 5H), 7.02-6.96 (m, 2H), 6.78-6.74 (m, 2H), 6.49 (d, J=9.3 Hz, 1H), 5.08 (q, J=7.0 Hz, 1H), 4.70 (t, J=8.0 Hz, 1H), 4.53-4.47 (m, 2H), 4.08 (d, J=11.5 Hz, 1H), 3.82 (dq, J=8.4, 4.2 Hz, 1H), 3.75 (t, J=6.1 Hz, 2H), 3.72-3.56 (m, 7H), 3.18 (t, J=5.1 Hz, 4H), 3.13-3.01 (m, 7H), 2.97 (dd, J=13.8, 7.6 Hz, 1H), 2.83-2.46 (m, 30H), 2.46-2.39 (m, 1H), 2.35 (t, J=5.1 Hz, 4H), 2.29-2.15 (m, 5H), 2.15-2.03 (m, 2H), 1.88 (d, J=17.8 Hz, 1H), 1.70-1.55 (m, 2H), 1.50 (d, J=7.0 Hz, 3H), 1.43 (dt, J=11.9, 5.5 Hz, 1H), 1.06 (s, 9H), 0.94 (s, 3H) ppm. LC/MS (ESI) m/z 1802.9; [M+H]⁺ calcd for C₉₀H₁₁₆ClF₃N₁₅O₁₁S₄⁺: 1802.75.

### Example 60: Preparation of Compound #112

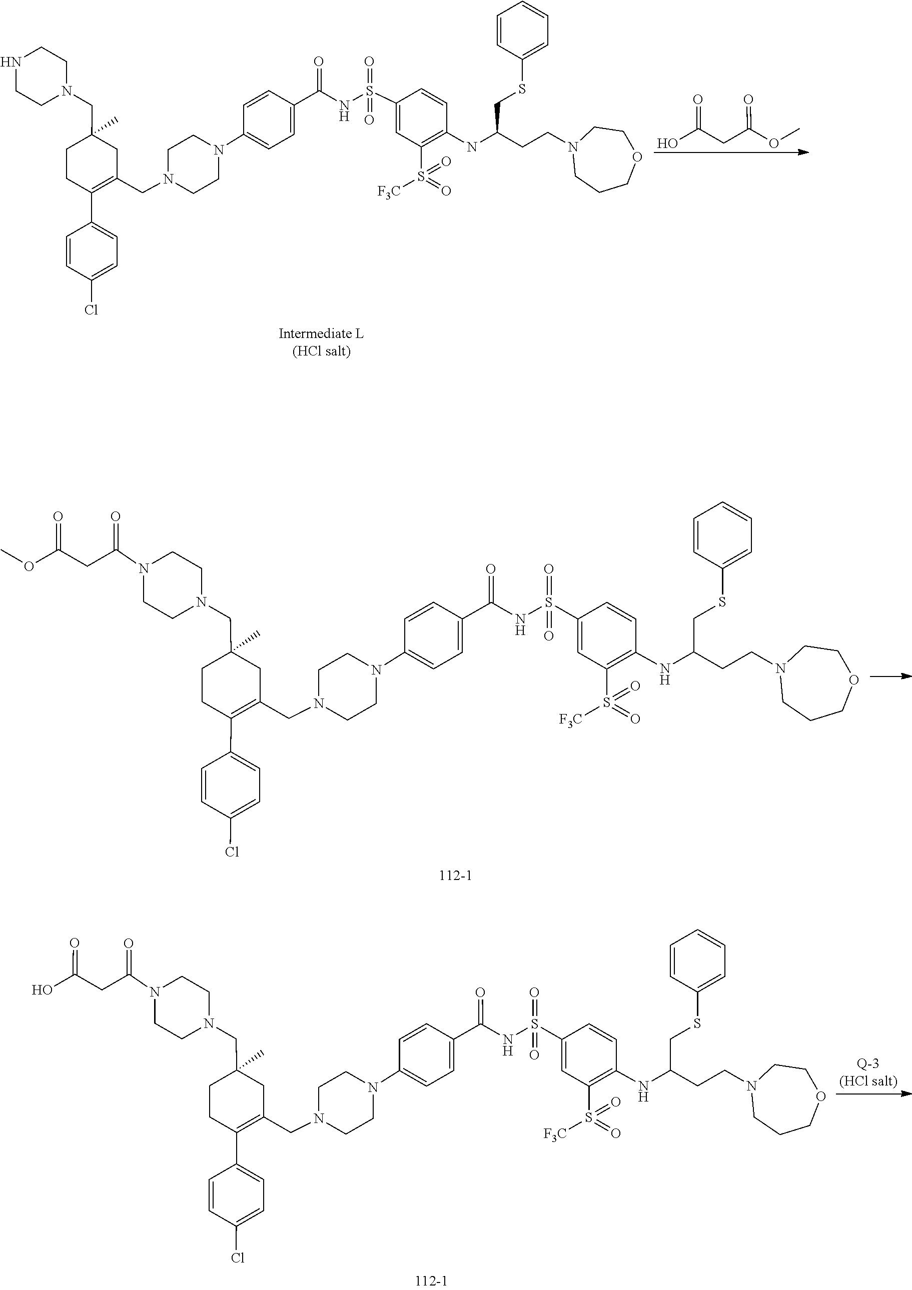

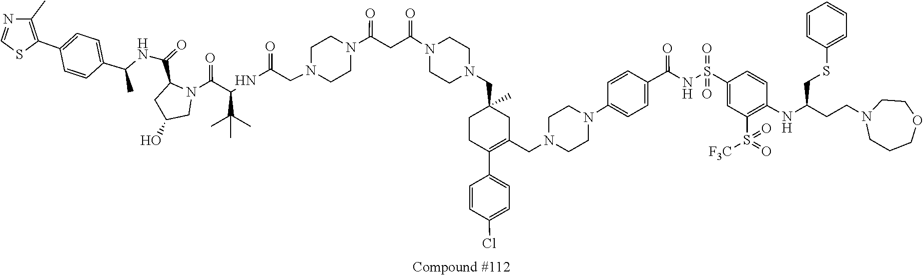 [SMILES: Cc1ncsc1-c1ccc([C@H](C)NC(=O)[C@@H]2C[C@@H](O)CN2C(=O)[C@@H](NC(=O)CN2CCN(C(=O)CC(=O)N3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1]

Preparation of methyl 3-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)-3-oxopropanoate (112-1): A solution of Intermediate L (12 mg, 0.011 mmol, HCl salt), 3-methoxy-3-oxopropanoic acid (1.9 mg, 0.016 mmol), TEA (20 μL, 0.27 mmol), and HATU (5.3 mg, 0.014 mmol) in DCM (0.3 mL) was stirred at room temperature for 1 hour, then washed with sat. aq. NH₄Cl. The organic layers were dried over Na₂SO₄, filtered, and concentrated. The residue was purified by preparative TLC (DCM/MeOH) to afford 112-1 (10.3 mg, 81% yield). ¹H NMR (600 MHz, CDCl₃) δ 8.36 (d, J=2.3 Hz, 1H), 8.06 (dd, J=9.2, 2.3 Hz, 1H), 7.73 (d, J=8.8 Hz, 2H), 7.42-7.37 (m, 2H), 7.34-7.23 (m, 5H), 7.11 (d, J=8.5 6 Hz, 1H), 7.02-6.98 (m, 2H), 6.79 (d, J=8.7 Hz, 2H), 6.59 (d, J=9.4 Hz, 1H), 3.92 (d, J=10.1 Hz, 1H), 3.82-3.71 (m, 6H), 3.65 (s, 2H), 3.52 (t, J=5.1 Hz, 2H), 3.29 (t, J=5.2 Hz, 4H), 3.11 (dd, J=13.9, 5.0 Hz, 1H), 3.06 (dd, J=13.9, 6.8 Hz, 1H), 2.93-2.62 (m, 7H), 2.56 (d, J=5.5 Hz, 4H), 2.42 (p, J=6.4 Hz, 4H), 2.37-2.21 (m, 3H), 2.18-2.08 (m, 2H), 1.97 (d, J=17.8 Hz, 3H), 1.78 (dq, J=13.6, 6.8 Hz, 1H), 1.66-1.58 (m, 2H), 1.52-1.44 (m, 3H), 1.34 (q, J=4.1 Hz, 2H), 0.97 (s, 3H) ppm. LC/MS (ESI) m/z 1172.2; [M+H]⁺ calcd for C₅₆H₇₀ClF₃N₇O₉S₃⁺: 1172.40.

Preparation of 3-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-3-oxopropanoic acid (112-2): To a solution of 112-1 (10 mg, 0.0085 mmol) in MeOH/THF (0.3 mL/0.3 mL) was added a solution of LiOH·H2O (3.6 mg, 0.086 mmol) in water (0.2 mL), then stirred at room temperature for 2 hours. The reaction mixture was adjusted to pH=5-6 with N aq. HCl, then concentrated. The residue was dried under vacuum to afford crude 112-2 (16 mg), which was used directly in the next step. Preparation of (2S,4R)-1-((S)-2-(2-(4-(3-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-3-oxopropanoyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound #112): A solution of 112-2 (16 mg, crude), Q-3 (6.2 mg, 0.01 mmol, HCl salt), TEA (10 μL, 0.07 mmol), and HATU (4.2 mg, 0.011 mmol) in DCM (0.5 mL). The resulting mixture was stirred at room temperature for 2 hours, then washed with sat. aq. NH₄Cl. The organic layers were dried over Na₂SO₄, filtered, and concentrated. The residue was purified by preparative TLC (DCM/MeOH=10/1) to afford compound #112 (1.7 mg, 12% yield). ¹H NMR (600 MHz, Chloroform-d) δ 8.67 (s, 1H), 8.33 (s, 1H), 8.11 (d, J=9.1 Hz, 1H), 7.70 (d, J=8.6 Hz, 2H), 7.42-7.31 (m, 7H), 7.31-7.19 (m, 5H), 6.97 (d, J=8.0 Hz, 2H), 6.76 (d, J=8.6 Hz, 2H), 6.64 (s, 1H), 5.07 (p, J=7.0 Hz, 1H), 4.69 (t, J=8.0 Hz, 1H), 4.54 (s, 1H), 4.48 (s, 1H), 4.06 (d, J=10.4 Hz, 1H), 4.01-3.87 (m, 1H), 3.86-3.68 (m, 6H), 3.68-3.52 (m, 9H), 3.52-3.39 (m, 3H), 3.25 (s, 5H), 3.15-2.97 (m, 6H), 2.95-2.64 (m, 5H), 2.52 (d, J=16.7 Hz, 9H), 2.47-2.13 (m, 10H), 2.09 (dd, J=13.8, 8.1 4 Hz, 1H), 2.01-1.71 (m, 5H), 1.61 (p, J=7.5 Hz, 3H), 1.47 (d, J=6.9 Hz, 3H), 1.04 (s, 9H), 0.94 (s, 3H) ppm. LC/MS (ESI) m/z 1710.8; [M+H]⁺ calcd for C₈₄H₁₀₈ClF₃N₁₃O₁₂S₄⁺: 1710.68.

### Example 61: Preparation of Compound #113

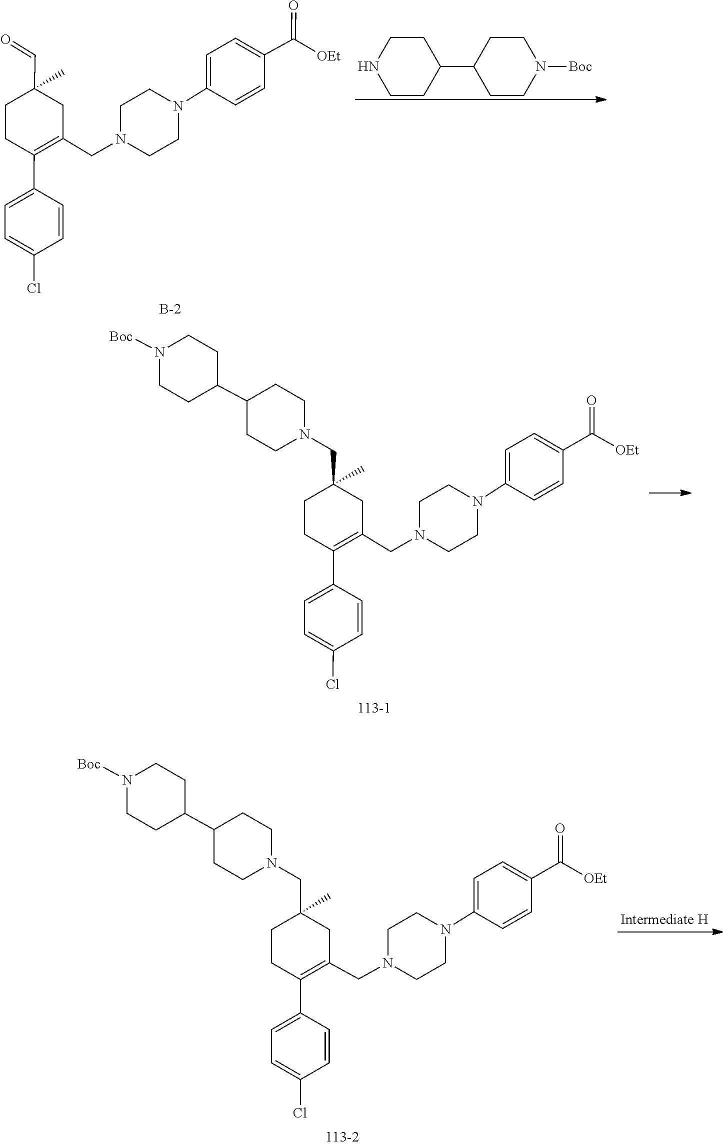

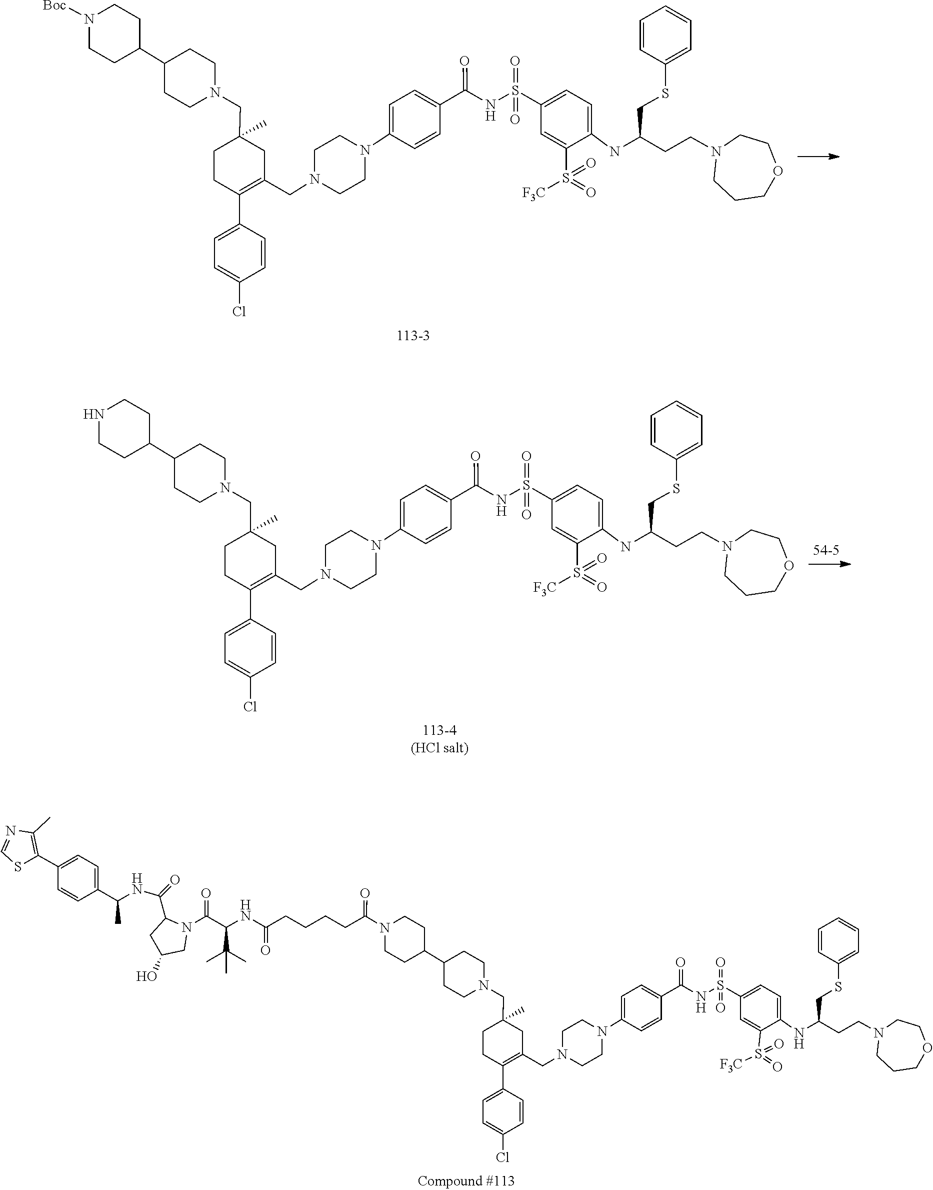

Compound #113 was prepared by following General Procedure F. tert-butyl (R)-1′-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-[4,4′-bipiperidine]-1-carboxylate (113-1) (470 mg, 88% yield). ¹H NMR (600 MHz, Chloroform-d) δ 7.95-7.88 (m, 2H), 7.33-7.26 (m, 2H), 7.04-6.99 (m, 2H), 6.86-6.80 (m, 2H), 4.34 (q, J=7.1 Hz, 2H), 4.13 (s, 2H), 3.28 (q, J=5.2, 4.0 Hz, 4H), 2.91-2.78 (m, 4H), 2.70-2.60 (m, 2H), 2.43-2.11 (m, 9H), 1.92 (d, J=17.4 Hz, 1H), 1.68 (d, J=12.6 Hz, 2H), 1.64-1.54 (m, 6H), 1.48 (s, 9H), 1.38 (t, J=7.1 Hz, 3H), 1.35-1.25 (m, 2H), 1.24-1.09 (m, 3H), 1.07-0.99 (m, 1H), 0.95 (s, 3H) ppm. LC/MS (ESI) m/z 733.5; [M+H]⁺ calcd for C₄₃H₆₂ClN₄04⁺: 733.45.

(R)-4-(4-((4-((1′-(tert-butoxycarbonyl)-[4,4′-bipiperidin]-1-yl)methyl)-4′-chloro-4-methyl-3,4,5,6-tetrahydro-[1,1′-biphenyl]-2-yl)methyl)piperazin-1-yl)benzoic acid (113-2) (385 mg, 91% yield). ¹H NMR (600 MHz, Methanol-d₄) δ 7.89-7.84 (m, 2H), 7.38-7.35 (m, 2H), 7.14-7.11 (m, 2H), 6.92-6.88 (m, 2H), 4.11 (d, J=13.1 Hz, 2H), 3.45-3.38 (m, 2H), 3.28 (t, J=5.1 Hz, 4H), 3.02-2.94 (m, 2H), 2.92-2.83 (m, 4H), 2.83-2.67 (m, 2H), 2.50 (t, J=5.0 Hz, 4H), 2.45-2.37 (m, 1H), 2.37-2.30 (m, 2H), 2.21 (d, J=17.2 Hz, 1H), 1.85 (d, J=13.9 Hz, 2H), 1.78-1.68 (m, 3H), 1.66-1.57 (m, 3H), 1.46 (s, 9H), 1.37-1.27 (m, 2H), 1.21-1.08 (m, 5H) ppm. LC/MS (ESI) m/z 705.6; [M+H]⁺ calcd for C₄₁H₅₈ClN₄O₄⁺: 705.41.

tert-butyl 1′-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidine]-1-carboxylate (113-3) (206 mg, 46% yield). ¹H NMR (600 MHz, Methanol-d₄) δ 7.89-7.84 (m, 2H), 7.38-7.35 (m, 2H), 7.14-7.11 (m, 2H), 6.92-6.88 (m, 2H), 4.11 (d, J=13.1 Hz, 2H), 3.45-3.38 (m, 2H), 3.28 (t, J=5.1 Hz, 4H), 3.02-2.94 (m, 2H), 2.92-2.83 (m, 4H), 2.83-2.67 (m, 2H), 2.50 (t, J=5.0 Hz, 4H), 2.45-2.37 (m, 1H), 2.33 (d, J=18.1 Hz, 2H), 2.21 (d, J=17.2 Hz, 1H), 1.85 (d, J=13.9 Hz, 2H), 1.78-1.68 (m, 3H), 1.66-1.57 (m, 3H), 1.46 (s, 9H), 1.37-1.27 (m, 2H), 1.21-1.08 (m, 5H) ppm. LC/MS (ESI) m/z 1254.3; [M+H]⁺ calcd for C₆₄H₈₅ClF₃N₆O₈S₃⁺: 1254.52.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4-([4,4′-bipiperidin]-1-ylmethyl)-4′-chloro-4-methyl-3,4,5,6-tetrahydro-[1,1′-biphenyl]-2-yl)methyl)piperazin-1-yl)benzamide hydrochloride (113-4) (61 mg, quantitative yield). LC/MS (ESI) m/z 1154.7; [M+H]⁺ calcd for C₅₈H₇₆ClF₃N₇O₆S₃⁺: 1154.47.

(2S,4R)-1-((S)-2-(6-(1′-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (compound #113) (6.4 mg, 22% yield). ¹H NMR (600 MHz, Chloroform-d) δ 8.69 (d, J=1.7 Hz, 1H), 8.36 (dd, J=5.9, 2.2 Hz, 1H), 8.09 (t, J=8.6 Hz, 1H), 7.84 (t, J=9.6 Hz, 2H), 7.49 (dd, J=34.5, 8.7 Hz, 1H), 7.44-7.35 (m, 6H), 7.30 (s, 5H), 7.09-7.03 (m, 1H), 7.03-6.98 (m, 2H), 6.77 (d, J=8.5 Hz, 2H), 6.54 (d, J=9.0 Hz, 2H), 5.10 (p, J=7.1 Hz, 1H), 4.79 (q, J=8.2 Hz, 1H), 4.59 (t, J=8.6 Hz, 1H), 4.51 (s, 1H), 4.15 (d, J=11.4 Hz, 1H), 3.92-3.64 (m, 6H), 3.60 (d, J=10.8 Hz, 1H), 3.22 (s, 4H), 3.16-2.99 (m, 2H), 2.97-2.80 (m, 2H), 2.80-2.51 (m, 9H), 2.50-2.18 (m, 23H), 2.11 (d, J=12.2 Hz, 2H), 1.90 (s, 2H), 1.79-1.53 (m, 10H), 1.49 (d, J=6.9 Hz, 4H), 1.28 (s, 5H), 1.07 (s, 12H) ppm. LC/MS (ESI) m/z 1708.7; [M+H]⁺ calcd for C₈₇H₁₁₄ClF₃N₁₁O₁₁S₄⁺: 1708.72.

### Example 62: Preparation of Compound #114

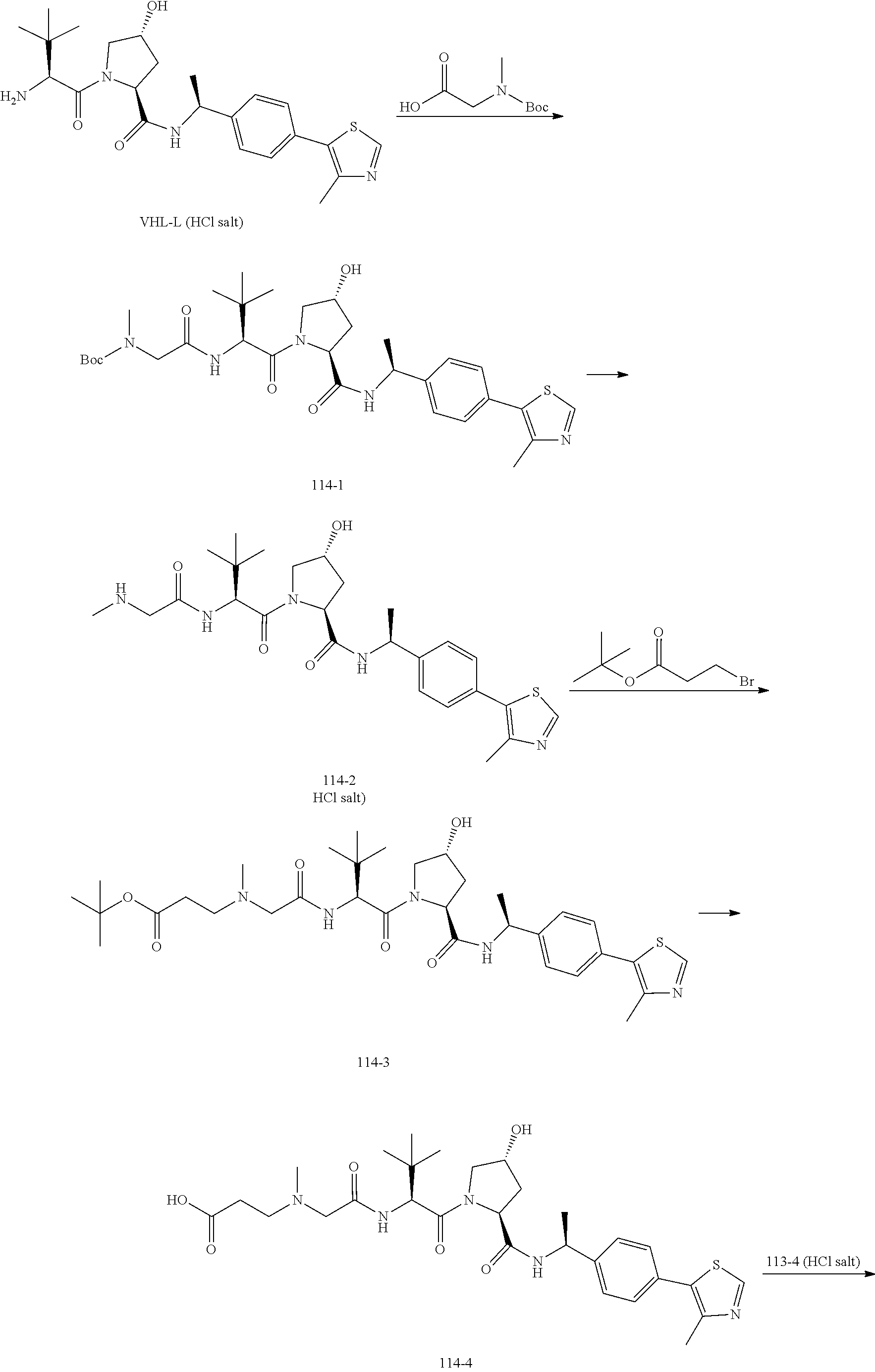

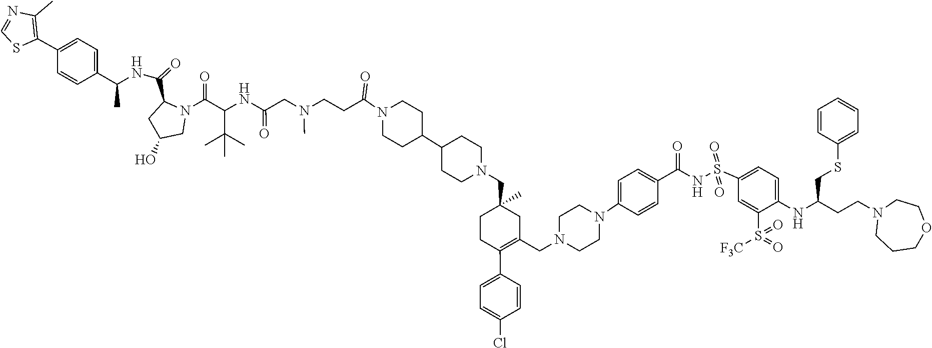 [SMILES: Cc1ncsc1-c1ccc([C@H](C)NC(=O)[C@@H]2C[C@@H](O)CN2C(=O)C(NC(=O)CN(C)CCC(=O)N2CCC(C3CCN(C[C@]4(C)CCC(c5ccc(Cl)cc5)=C(CN5CCN(c6ccc(C(=O)NS(=O)(=O)c7ccc(N[C@H](CCN8CCCOCC8)CSc8ccccc8)c(S(=O)(=O)C(F)(F)F)c7)cc6)CC5)C4)CC3)CC2)C(C)(C)C)cc1]

Preparation of tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate (114-1): A solution of VHL-L (400 mg, 0.83 mmol, HCl salt), and TEA (347 μL, 2.50 mmol) in DCM (5 mL) was added to a solution of N-(tert-butoxycarbonyl)-N-methylglycine (189 mg, 1.0 mmol), and HATU (632 mg, 1.66 mmol) in DCM (5 mL). The resulting mixture was stirred at room temperature overnight. The reaction mixture was washed with sat. aq. NH₄Cl, dried over Na₂SO₄, filtered, and concentrated. The residue was purified by flash column chromatography (0% to 10% of MeOH in DCM) to afford 114-1 (430 mg, 84% yield). LC/MS (ESI) m/z 616.5; [M+H]⁺ calcd for C₃₁H₄₆N₅O₆S⁺: 616.32.

Preparation of (2S,4R)-1-((S)-3,3-dimethyl-2-(2-(methylamino)acetamido)butanoyl)-4-hydroxy-N—((S)-1-(4-(4-methylthiazol-5-yl)phenyl)ethyl)pyrrolidine-2-carboxamide hydrochloride (114-2): To a solution of 114-1 (200 mg, 0.32 mmol) in DCM (4 mL) was added 4N HCl in dioxane (4 mL), then stirred at room temperature for 1 hour. The reaction mixture was concentrated to afford 114-2 (170 mg, quantitative yield), which was used directly in the next step. LC/MS (ESI) m/z 516.5; [M+H]⁺ calcd for C₂₆H₃₈N₅O₄S⁺: 516.26.

Preparation of tert-butyl 3-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)amino)propanoate (114-3): A solution of 114-2 (57 mg, 0.10 mmol), tert-butyl 3-bromopropanoate (65 mg, 0.31 mmol), and DIPEA (144 μL, 0.83 mmol) in DCM (2 mL) was stirred at room temperature overnight. The reaction mixture was washed with sat. aq. NH₄Cl. The organic layer was dried over Na₂SO₄, filtered, and concentrated. The residue was purified by flash column chromatography (0% to 10% of MeOH in DCM) to afford 114-3 (10 mg, 24% yield). ¹H NMR (600 MHz, Chloroform-d) δ 8.69 (s, 1H), 7.87 (s, 1H), 7.58 (s, 1H), 7.44-7.36 (m, 4H), 5.10 (p, J=7.1 Hz, 1H), 4.81 (q, J=7.7, 6.2 Hz, 1H), 4.50 (d, J=7.1 Hz, 2H), 4.19 (s, 1H), 3.59 (dd, J=11.7, 3.3 Hz, 1H), 3.11 (s, 3H), 2.81 (s, 3H), 2.55 (s, 3H), 2.46 (s, 2H), 2.36 (s, 3H), 2.10 (s, 1H), 1.51-1.42 (m, 12H), 1.09 (d, J=2.7 Hz, 9H) ppm. LC/MS (ESI) m/z 644.1; [M+H]⁺ calcd for C₃₃H₅₀N₅O₆S⁺: 644.35.

Preparation of 3-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)amino)propanoic acid (114-4): To a solution of 114-3 (10 mg, 0.016 mmol) in DCM (0.5 mL) was added 4N HCl in dioxane (0.5 mL), then stirred at room temperature overnight. The reaction mixture was concentrated to afford 114-4 (9 mg, quantitative yield), which was used directly in the next step. LC/MS (ESI) m/z 588.1; [M+H]⁺ calcd for C₂₉H₄₂N₅O₆S⁺: 588.29

Preparation of (2S,4R)-1-((S)-2-(2-((3-(1′-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-3-oxopropyl)(methyl)amino)acetamido)-3,3-dimethylbutanoyl)-4-hydroxy-N—((S)-1-(4-(4-methylthiazol-5-yl)phenyl)ethyl)pyrrolidine-2-carboxamide (compound #114): A solution of 113-4 (9 mg, 0.0076 mmol, HCl salt), and TEA (6.3 μL, 0.045 mmol) in DMF (0.2 mL) was added to a solution of 54-5 (4.3 mg, 0.0076 mmol), and HATU (4.3 mg, 0.011 mmol) in DMF (0.2 mL). The resulting mixture was stirred at room temperature for 2 hours. The reaction mixture was diluted with EtOAc and washed with water and sat. aq. NH₄Cl. The organic layer was dried over Na₂SO₄, filtered, and concentrated. The residue was purified by preparative TLC (DCM/MeOH) to afford compound #114 (1.9 mg, 15% yield). ¹H NMR (600 MHz, Chloroform-d) δ 8.67 (s, 1H), 8.28 (s, 1H), 8.01 (d, J=9.1 Hz, 1H), 7.79 (s, 2H), 7.43-7.19 (m, 10H), 7.09-6.97 (m, 3H), 6.84 (d, J=8.5 Hz, 1H), 6.73-6.54 (m, 3H), 5.10-4.99 (m, 1H), 4.66 (s, 1H), 4.63-4.39 (m, 4H), 4.04 (d, J=11.5 Hz, 2H), 4.00-3.84 (m, 2H), 3.76 (t, J=6.0 Hz, 2H), 3.68 (s, 2H), 3.60 (d, J=11.1 Hz, 1H), 3.42 (p, J=1.7 Hz, 2H), 3.30-3.03 (m, 4H), 2.84-2.43 (m, 9H), 2.41-2.22 (m, 8H), 1.72 (d, J=125.1 Hz, 32H), 1.53-1.44 (m, 4H), 1.07-0.99 (m, 12H), 0.88 (t, J=7.0 Hz, 3H) ppm. LC/MS (ESI) m/z 1723.5; [M+H]⁺ calcd for C₈₇H₁₁₅ClF₃N₁₂O₁₁S₄⁺: 1723.73.

### Example 63: Preparation of Compound #115

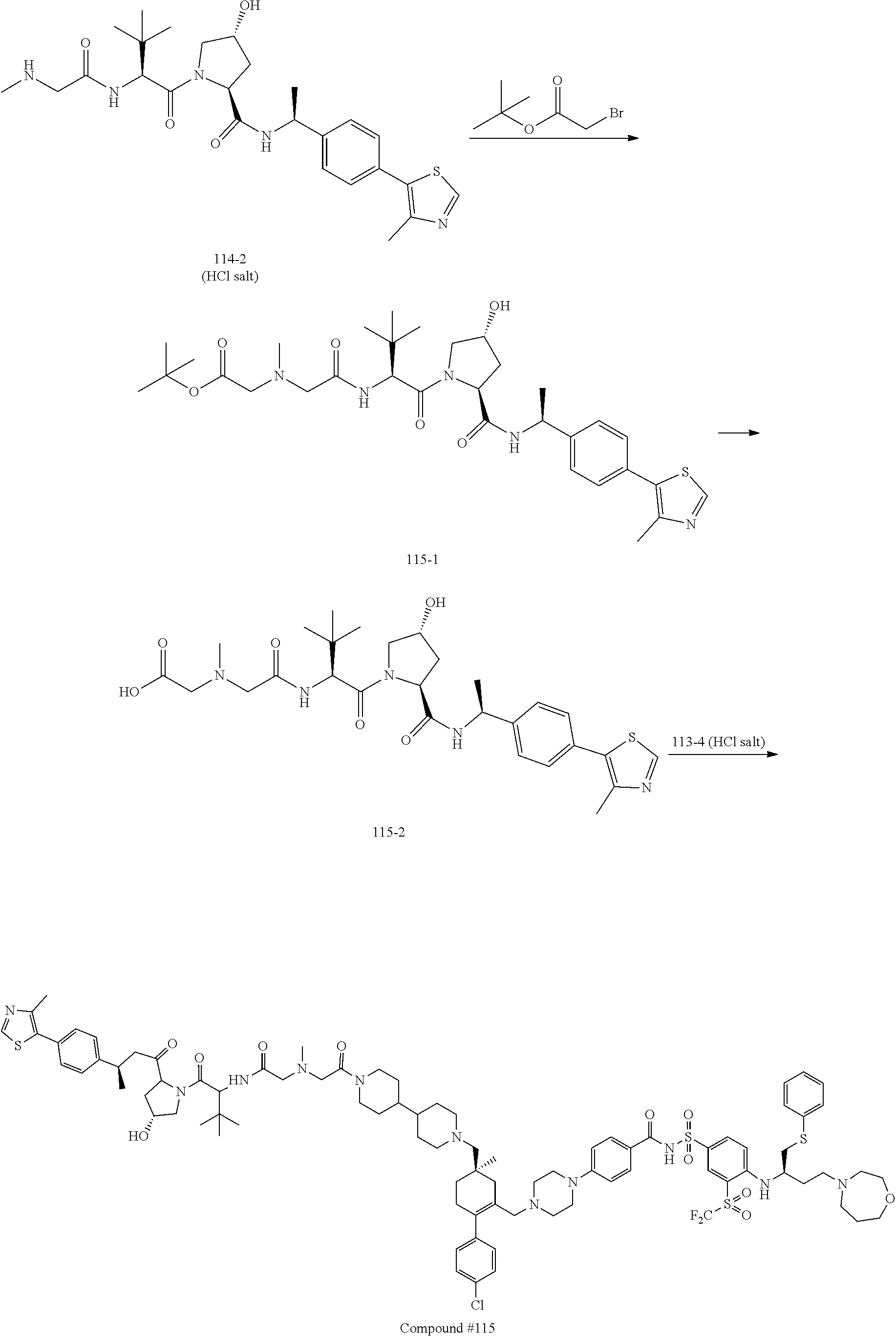

Preparation of tert-butyl N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycinate (115-1): A solution of 114-2 (112 mg, 0.20 mmol), tert-butyl 2-bromoacetate (119 mg, 0.61 mmol), and DIPEA (353 μL, 2.03 mmol) in DCM (5 mL) was stirred at room temperature for 2 days. The reaction mixture was washed with sat. aq. NH₄Cl, then dried over Na₂SO₄, filtered, and concentrated. The residue was purified by flash column chromatography (0% to 10% of MeOH in DCM) to afford 115-1 (83 mg, 65% yield). ¹H NMR (600 MHz, Chloroform-d) δ 8.69 (s, 1H), 8.05 (d, J=8.1 Hz, 1H), 7.59 (d, J=7.8 Hz, 1H), 7.45-7.35 (m, 4H), 5.10 (p, J=7.1 Hz, 1H), 4.80 (t, J=7.9 Hz, 1H), 4.52 (d, J=4.8 Hz, 1H), 4.47 (dd, J=8.1, 1.7 Hz, 1H), 4.22 (dd, J=11.9, 4.2 Hz, 1H), 3.60 (dd, J=11.5, 3.6 Hz, 1H), 3.30-3.15 (m, 3H), 2.65-2.57 (m, 1H), 2.55 (s, 3H), 2.46 (s, 3H), 2.08 (ddt, J=13.1, 8.3, 2.0 Hz, 1H), 1.87-1.73 (m, 2H), 1.49 (d, J=4.8 Hz, 12H), 1.11 (s, 9H) ppm. LC/MS (ESI) m/z 630.5; [M+H]⁺ calcd for C₃₂H₄₈N₅O₆S⁺: 630.33.

Preparation of N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycine (115-2): To a solution of 115-1 (50 mg, 0.079 mmol) in DCM (1.0 mL) was added 4N HCl in dioxane (2.0 mL), then stirred at room temperature overnight. The reaction mixture was concentrated to afford 115-2 (52 mg, quantitative yield), which was used directly in the next step. LC/MS (ESI) m/z 574.1; [M+H]⁺ calcd for C₂₈H₄₀N₅O₆S⁺: 574.27.

Preparation of (2S,4R)-1-((S)-2-(2-((2-(1′-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-[4,4′-bipiperidin]-1-yl)-2-oxoethyl)(methyl)amino)acetamido)-3,3-dimethylbutanoyl)-4-hydroxy-N—((S)-1-(4-(4-methylthiazol-5-yl)phenyl)ethyl)pyrrolidine-2-carboxamide (compound #115): A solution of 113-4 (30 mg, 0.025 mmol, HCl salt), and TEA (28 μL, 0.20 mmol) in DMF (0.5 mL) was added to a solution of 115-4 (22 mg, 0.038 mmol), and HATU (19 mg, 0.050 mmol) in DMF (0.5 mL). The resulting mixture was stirred at room temperature for 2 hours. The reaction mixture was diluted with EtOAc and washed with water and sat. aq. NH₄Cl. The organic layer was dried over Na₂SO₄, filtered, and concentrated. The residue was purified by preparative TLC (DCM/MeOH=10/1) to afford compound #115 (5.5 mg, 13% yield). ¹H NMR (600 MHz, Chloroform-d) δ 8.69 (d, J=1.9 Hz, 1H), 8.37-8.30 (m, 1H), 8.17 (s, 1H), 8.04 (d, J=9.1 Hz, 1H), 7.86 (d, J=8.4 Hz, 2H), 7.56 (d, J=7.8 Hz, 1H), 7.43-7.35 (m, 6H), 7.34-7.21 (m, 5H), 7.00 (d, J=8.2 Hz, 3H), 6.75 (t, J=7.7 Hz, 2H), 6.49 (d, J=9.3 Hz, 1H), 5.09 (p, J=7.1 Hz, 1H), 4.80 (t, J=8.0 Hz, 1H), 4.60-4.45 (m, 3H), 4.18 (t, J=12.7 Hz, 1H), 3.87-3.66 (m, 6H), 3.60 (dd, J=11.6, 3.6 Hz, 1H), 3.37 (s, 2H), 3.29-2.98 (m, 9H), 2.98-2.56 (m, 8H), 2.56-2.19 (m, 22H), 2.11 (d, J=11.9 Hz, 3H), 1.93 (s, 2H), 1.81-1.40 (m, 11H), 1.36-1.23 (m, 5H), 1.17-0.98 (m, 12H) ppm. LC/MS (ESI) m/z 1709.6; [M+H]⁺ calcd for C₈₆H₁₁₃ClF₃N₁₂O₁₁S₄⁺: 1709.72.

### Example 64: Preparation of Compound #116

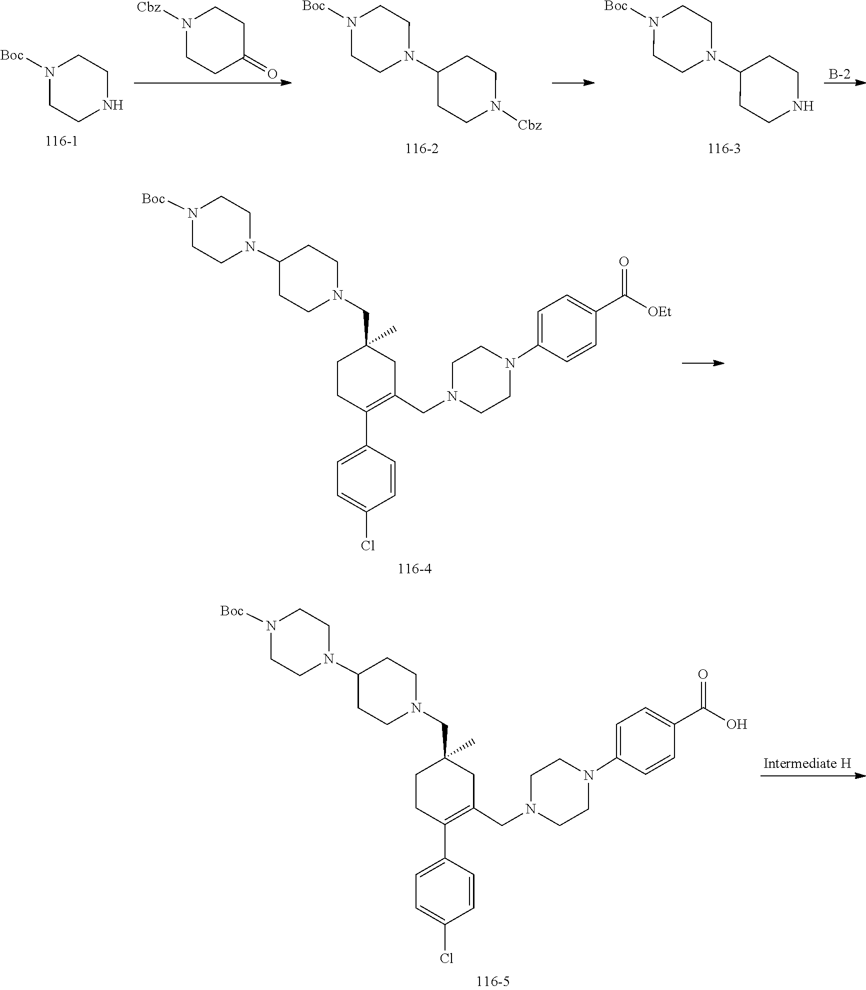

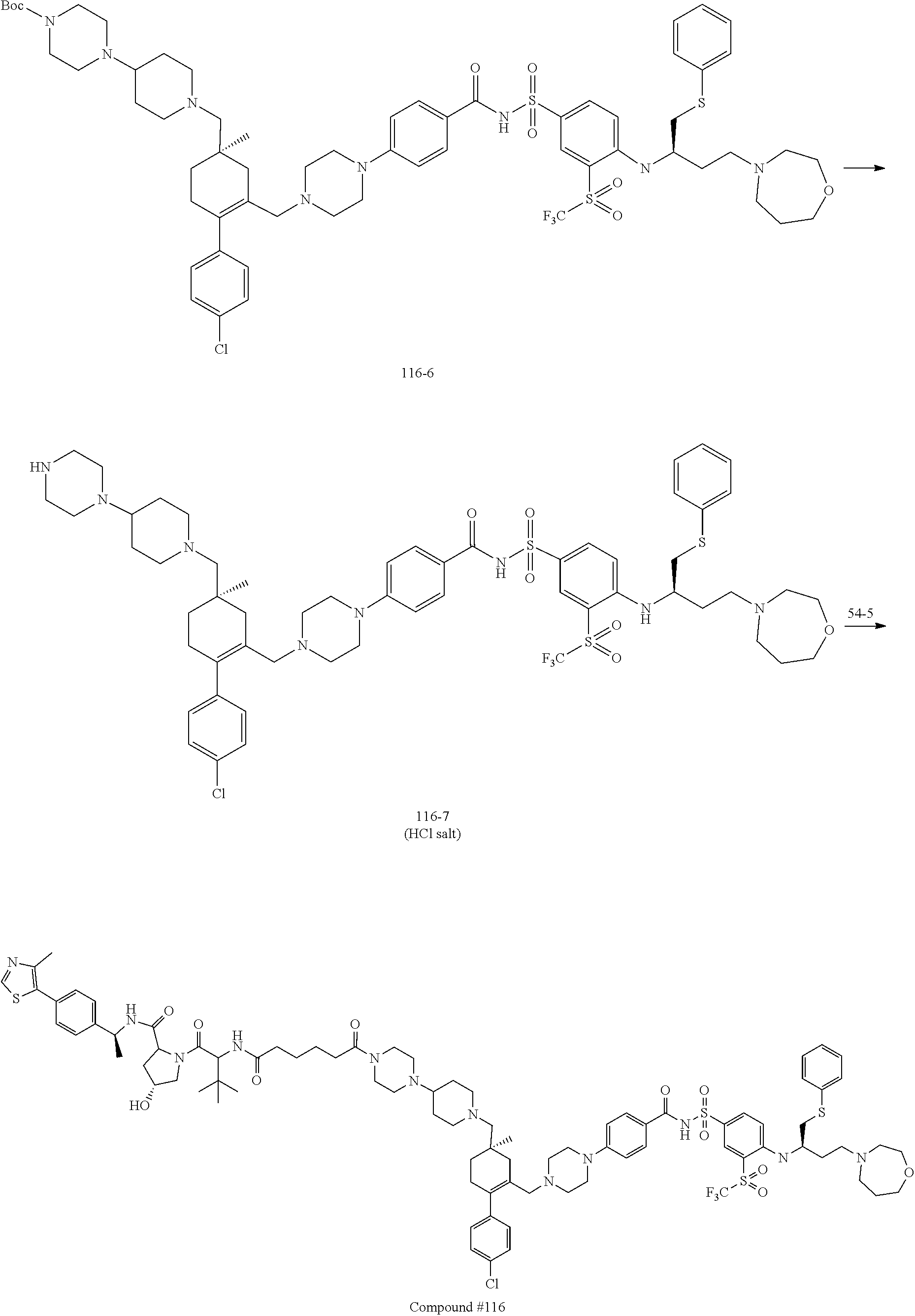

Preparation of tert-butyl 4-(1-((benzyloxy)carbonyl)piperidin-4-yl)piperazine-1-carboxylate (116-2): To a solution of 116-1 (186 mg, 1.0 mmol), and benzyl 4-oxopiperidine-1-carboxylate (233 mg, 1.0 mmol) in DCM (5 mL) was added NaBH(OAc)₃ (424 mg, 2.0 mmol). The resulting mixture was stirred at room temperature overnight, then washed with sat. aq. NH₄Cl. The organic layer was dried over Na₂SO₄, filtered, and concentrated. The residue was purified by flash column chromatography (0% to 80% of hexanes in EtOAc) to afford 116-2 (200 mg, 50% yield). ¹H NMR (600 MHz, Chloroform-d) δ 7.41-7.30 (m, 5H), 5.14 (s, 2H), 4.25 (s, 2H), 3.44 (t, J=5.0 Hz, 4H), 2.81 (s, 2H), 2.51 (t, J=4.8 Hz, 4H), 2.47-2.40 (m, 1H), 1.81 (s, 2H), 1.52-1.40 (m, 11H) ppm. LC/MS (ESI) m/z 404.3; [M+H]⁺ calcd for C₂₂H₃₄N₃O₄⁺: 404.25.

Preparation of tert-butyl 4-(piperidin-4-yl)piperazine-1-carboxylate (116-3): A stirring suspension of 116-2 (190 mg, 0.47 mmol), and 10% Pd/C (30 mg) in EtOAc/MeOH (4 mL/4 mL) was hydrogenated with a hydrogen balloon overnight. The reaction mixture was filtered through celite, the filtrate was concentrated to afford crude 116-3 (95 mg, 75% yield), which was used in the next step without further purification. LC/MS (ESI) m/z 270.5; [M+H]⁺ calcd for C₁₄H₂₈N₃O₂⁺: 270.22.

The last five steps for the preparation of compound #116 were carried out according to General Procedure F.

tert-butyl (R)-4-(1-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)piperidin-4-yl)piperazine-1-carboxylate (116-4) (120 mg, 55% yield). ¹H NMR (600 MHz, Chloroform-d) δ 7.94-7.89 (m, 2H), 7.28 (s, 2H), 7.03-6.99 (m, 2H), 6.85-6.81 (m, 2H), 4.34 (q, J=7.1 Hz, 2H), 3.45 (t, J=4.9 Hz, 4H), 3.27 (t, J=5.1 Hz, 4H), 2.89 (d, J=11.3 Hz, 2H), 2.81 (s, 2H), 2.52 (s, 4H), 2.41-2.33 (m, 4H), 2.33-2.10 (m, 6H), 1.92 (d, J=17.3 Hz, 1H), 1.73 (d, J=12.1 Hz, 2H), 1.68-1.53 (m, 4H), 1.51-1.42 (m, 10H), 1.38 (t, J=7.1 Hz, 3H), 0.95 (s, 3H) ppm. LC/MS (ESI) m/z 734.6; [M+H]⁺ calcd for C₄₂H₆₁ClN₅O₄⁺: 734.44.

(R)-4-(4-((4-((4-(4-(tert-butoxycarbonyl)piperazin-1-yl)piperidin-1-yl)methyl)-4′-chloro-4-methyl-3,4,5,6-tetrahydro-[1,1′-biphenyl]-2-yl)methyl)piperazin-1-yl)benzoic acid (116-5) (98 mg, 85% yield). ¹H NMR (600 MHz, Methanol-d₄) δ 7.87 (d, J=9.0 Hz, 2H), 7.38-7.35 (m, 2H), 7.15-7.11 (m, 2H), 6.93-6.90 (m, 2H), 3.50 (s, 4H), 3.33 (s, 4H), 3.07 (q, J=12.6 14 Hz, 2H), 2.85-2.64 (m, 10H), 2.57 (s, 6H), 2.47-2.27 (m, 3H), 2.16 (d, J=17.0 Hz, 1H), 2.00-1.93 (m, 2H), 1.89 (t, J=12.3 Hz, 2H), 1.73-1.64 (m, 1H), 1.62-1.55 (m, 1H), 1.47 (s, 9H), 1.14 (s, 3H) ppm. LC/MS (ESI) m/z 706.4; [M+H]⁺ calcd for C₄₀H₅₇ClN₅O₄⁺: 706.41.

tert-butyl 4-(1-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperidin-4-yl)piperazine-1-carboxylate (116-6) (44 mg, 49% yield). ¹H NMR (600 MHz, CDCl₃) δ 8.36 (d, J=2.3 Hz, 1H), 8.03 (d, J=9.0 Hz, 1H), 7.82 (d, J=8.5 Hz, 2H), 7.39 (d, J=7.1 Hz, 2H), 7.29 (td, J=16.1, 7.5 Hz, 5H), 7.00 (d, J=8.0 Hz, 3H), 6.77 (d, J=8.5 Hz, 2H), 6.50 (d, J=9.3 Hz, 1H), 3.87 (d, J=12.1 Hz, 1H), 3.81-3.69 (m, 4H), 3.49 (s, 4H), 3.25 (s, 5H), 3.11 (dd, J=13.9, 4.9 Hz, 1H), 3.03 (dd, J=13.6, 6.9 Hz, 2H), 2.93-2.51 (m, 12H), 2.51-2.17 (m, 12H), 2.13 (d, J=10.2 Hz, 1H), 2.07-1.88 (m, 3H), 1.86-1.60 (m, 6H), 1.48 (s, 9H), 0.98 (s, 3H) ppm. LC/MS (ESI) m/z 1255.7; [M+H]⁺ calcd for C₆₂H₈₃ClF₃N₈O₈S₃⁺: 1255.51.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-((4-morpholinopiperidin-1-yl)methyl)-3,4,5,6-tetrahydro-[1,1′-biphenyl]-2-yl)methyl)piperazin-1-yl)benzamide hydrochloride (116-7) (19 mg, quantitative yield). LC/MS (ESI) m/z 1155.5; [M+H]⁺ calcd for C₅₇H₇₅ClF₃N₈O₆S₃⁺: 1155.46.

(2S,4R)-1-((S)-2-(6-(4-(1-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperidin-4-yl)piperazin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (compound #116) (4.4 mg, 22% yield in two steps). ¹H NMR (600 MHz, Chloroform-d) δ 8.69 (s, 1H), 8.35 (d, J=2.2 Hz, 1H), 8.08 (d, J=9.2 Hz, 1H), 7.81 (d, J=8.5 Hz, 2H), 7.49 (d, J=7.8 Hz, 1H), 7.44-7.35 (m, 6H), 7.34-7.22 (m, 5H), 7.08 (d, J=8.3 Hz, 1H), 7.01 (d, J=8.3 Hz, 2H), 6.77 (d, J=8.6 Hz, 2H), 6.56 (d, J=9.3 Hz, 1H), 6.50 (d, J=8.2 Hz, 1H), 5.10 (p, J=7.1 Hz, 1H), 4.78 (t, J=8.1 Hz, 1H), 4.58 (d, J=8.4 Hz, 1H), 4.51 (s, 1H), 4.15 (d, J=11.4 Hz, 1H), 3.90 (s, 1H), 3.78 (t, J=6.1 Hz, 2H), 3.74-3.65 (m, 2H), 3.59 (dd, J=11.4, 3.5 Hz, 1H), 3.47 (s, 2H), 3.24 (s, 4H), 3.18-3.08 (m, 2H), 3.04 (dd, J=13.9, 7.1 Hz, 1H), 3.00-2.83 (m, 3H), 2.78-2.20 (m, 28H), 2.13 (d, J=16.6 Hz, 2H), 1.90 (s, 3H), 1.82-1.55 (m, 8H), 1.48 (d, J=6.9 Hz, 3H), 1.39 (t, J=7.3 Hz, 8H), 1.07 (s, 9H), 0.96 (s, 3H) ppm. LC/MS (ESI) m/z 1709.9; [M+H]⁺ calcd for Cs₆H₁₁₃ClF₃N₁₂O₁₁S₄⁺: 1709.72.

### Example 65: Preparation of Compound #117

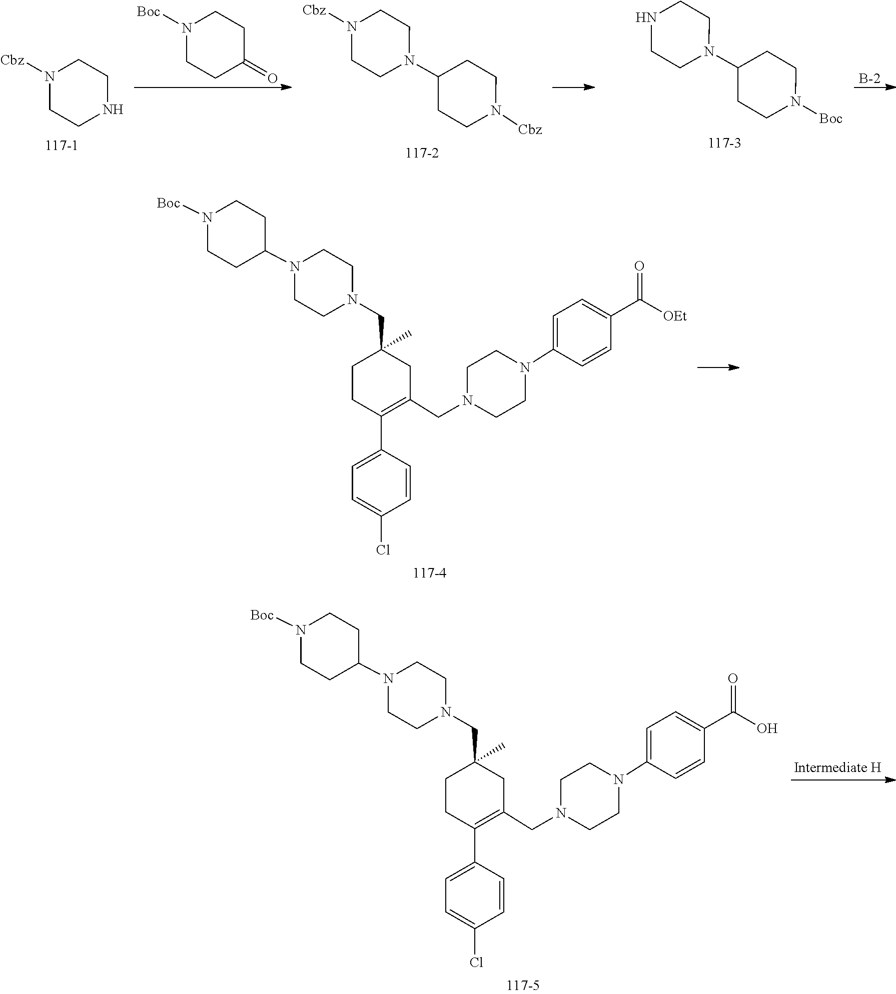

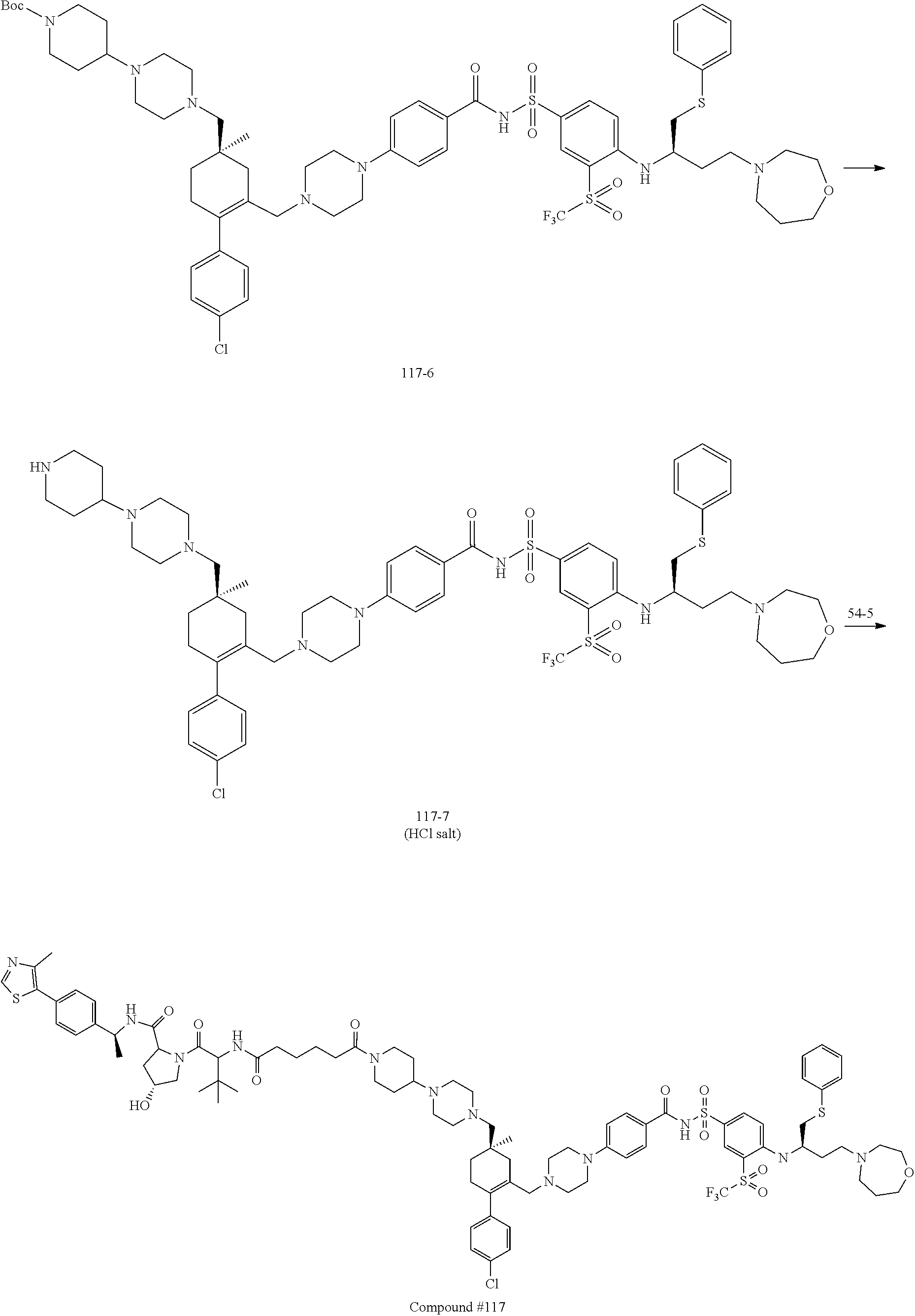

Preparation of benzyl 4-(1-(tert-butoxycarbonyl)piperidin-4-yl)piperazine-1-carboxylate (117-2): To a solution of 117-1 (220 mg, 1.0 mmol), and tert-butyl 4-oxopiperidine-1-carboxylate (199 mg, 1.0 mmol) in DCM (5 mL) was added NaBH(OAc)₃ (424 mg, 2.0 mmol), then stirred at room temperature overnight. The reaction mixture was washed with sat. aq. NH₄Cl. The organic layers were dried over Na₂SO₄, filtered, and concentrated. The residue was purified by flash column chromatography (0% to 80% of hexanes in EtOAc) to afford 116-2 (190 mg, 47% yield). ¹H NMR (600 MHz, CDCl₃) δ 7.36 (s, 5H), 5.15 (s, 2H), 4.16 (s, 1H), 3.53 (t, J 6=5.1 Hz, 4H), 2.71 (s, 2H), 2.54 (s, 5H), 2.42 (tt, J=11.4, 3.6 Hz, 1H), 1.78 (d, J=12.6 Hz, 2H), 1.47 (s, 9H), 1.42 (qd, J=12.2, 4.3 Hz, 2H) ppm. LC/MS (ESI) m/z 404.5; [M+H]⁺ calcd for C₂₂H₃₄N₃O₄⁺: 404.25.

Preparation of tert-butyl 4-(piperazin-1-yl)piperidine-1-carboxylate (117-3): A stirring suspension of 116-2 (180 mg, 0.45 mmol), and 10% Pd/C (30 mg) in EtOAc/MeOH (4 mL/4 12 mL) was hydrogenated with a hydrogen balloon overnight. The reaction mixture was filtered through celite, the filtrate was concentrated to afford crude 116-3 (102 mg, 85% yield), which was used directly in the next step. ¹H NMR (600 MHz, CDCl₃) δ 4.14 (s, 1H), 2.93 (t, J=4.9 Hz, 4H), 2.71 (s, 2H), 2.57 (t, J=4.9 Hz, 4H), 2.41-2.25 (m, 3H), 1.81 (d, J=12.5 Hz, 2H), 1.47 (s, 9H), 1.41 (qd, J=12.1, 4.3 Hz, 2H) ppm. LC/MS (ESI) m/z 270.3; [M+H]⁺ calcd for C₁₄H₂₈N₃O₂⁺: 270.22.

The last 5 steps for the preparation of compound #117 were carried out according to General Procedure F.

tert-butyl (R)-4-(4-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)piperazin-1-yl)piperidine-1-carboxylate (117-4) (126 mg, 57% yield). ¹H NMR (600 MHz, CDCl₃) δ 7.94-7.90 (m, 2H), 7.32-7.27 (m, 2H), 7.03-6.99 (m, 2H), 6.85-6.81 (m, 2H), 4.34 (q, J=7.1 Hz, 2H), 4.14 (s, 1H), 3.27 (t, J=5.2 Hz, 4H), 2.81 (s, 2H), 2.71 (s, 2H), 2.66-2.53 (m, 7H), 2.43-2.19 (m, 8H), 2.15 (dt, J=17.4, 2.4 Hz, 1H), 2.00 (s, 1H), 1.97-1.91 (m, 1H), 1.83 (d, J=12.5 Hz, 2H), 1.65-1.57 (m, 1H), 1.50-1.40 (m, 12H), 1.38 (t, J=7.1 Hz, 3H) ppm. LC/MS (ESI) m/z 734.6; [M+H]⁺ calcd for C₄₂H₆₁ClN₅O₄⁺: 734.44.

(R)-4-(4-((4-((4-(1-(tert-butoxycarbonyl)piperidin-4-yl)piperazin-1-yl)methyl)-4′-chloro-4-methyl-3,4,5,6-tetrahydro-[1,1′-biphenyl]-2-yl)methyl)piperazin-1-yl)benzoic acid (117-5) (110 mg, 91% yield). ¹H NMR (600 MHz, MeOD) δ 7.91-7.85 (m, 2H), 7.36 (d, J=8.3 Hz, 2H), 7.13-7.09 (m, 2H), 6.93 (d, J=8.9 Hz, 2H), 4.22 (d, J=13.3 Hz, 2H), 3.35 (t, J=5.2 Hz, 4H), 3.20-3.02 (m, 7H), 2.98-2.70 (m, 7H), 2.58 (s, 4H), 2.52-2.23 (m, 4H), 2.07 (d, J=13.8 Hz, 3H), 1.72-1.62 (m, 1H), 1.58-1.43 (m, 12H), 1.06 (s, 3H) ppm. LC/MS (ESI) m/z 706.5; [M+H]⁺ calcd for C₄₀H₅₇ClN₅O₄⁺: 706.41.

tert-butyl 4-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)piperidine-1-carboxylate (117-6) (41 mg, 46% yield). ¹H NMR (600 MHz, CDCl₃) δ 8.37 (d, J=2.2 Hz, 1H), 8.06-8.01 (m, 1H), 7.83 (d, J=8.5 Hz, 2H), 7.42-7.37 (m, 2H), 7.36-7.22 (m, 5H), 7.01 (t, J=9.7 Hz, 3H), 6.79 (d, J=8.5 Hz, 2H), 6.54 (d, J=9.3 Hz, 1H), 4.20 (s, 3H), 3.93-3.85 (m, 1H), 3.82-3.66 (m, 4H), 3.24 (s, 4H), 3.11 (dd, J=13.9, 4.9 Hz, 1H), 3.03 (dd, J=13.8, 7.3 Hz, 1H), 2.97-2.54 (m, 18H), 2.39 (s, 4H), 2.26 (s, 4H), 2.19-2.05 (m, 2H), 2.04-1.82 (m, 6H), 1.78-1.67 (m, 1H), 1.65-1.42 (m, 12H), 0.96 (s, 3H) ppm. LC/MS (ESI) m/z 1255.6; [M+H]⁺ calcd for C₆₂H₈₃ClF₃N₈O₈S₃⁺: 1255.51.

N-((4-(((R)-4-(1,4-oxazepan-4-yl)-1-(phenylthio)butan-2-yl)amino)-3-((trifluoromethyl)sulfonyl)phenyl)sulfonyl)-4-(4-(((R)-4′-chloro-4-methyl-4-((4-(piperidin-4-yl)piperazin-1-yl)methyl)-3,4,5,6-tetrahydro-[1,1′-biphenyl]-2-yl)methyl)piperazin-1-yl)benzamide hydrochloride (117-7) (18 mg, quantitative yield). LC/MS (ESI) m/z 1155.7; [M+H]⁺ calcd for C₅₇H₇₅ClF₃N₈O₆S₃⁺: 1155.46.

(2S,4R)-1-((S)-2-(6-(4-(4-(((R)-6-((4-(4-(((4-(((R)-4-(1,4-oxazepan-4-yl)-1-(phenylthio)butan-2-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)piperidin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (compound #117) (5.4 mg, 26% yield in two steps). ¹H NMR (600 MHz, CDCl₃) δ 8.69 (s, 1H), 8.35 (dd, J=5.9, 2.2 Hz, 1H), 8.06 (s, 1H), 7.85 (d, J=7.9 Hz, 2H), 7.55-7.35 (m, 7H), 7.34-7.23 (m, 5H), 7.05 (s, 1H), 7.00 (dd, J=8.5, 2.5 Hz, 2H), 6.78 (t, J=7.2 Hz, 2H), 6.59-6.50 (m, 2H), 5.10 (p, J=7.1 Hz, 1H), 4.76 (dt, J=14.6, 8.0 Hz, 1H), 4.70-4.56 (m, 2H), 4.50 (s, 1H), 4.08 (dd, J=22.9, 11.4 Hz, 1H), 3.90 (s, 2H), 3.77 (t, J=6.1 Hz, 2H), 3.75-3.65 (m, 2H), 3.60 (dd, J=11.3, 3.6 Hz, 1H), 3.24 (s, 4H), 3.17-3.08 (m, 3H), 3.04 (dd, J=13.9, 7.1 Hz, 1H), 2.97-2.57 (m, 12H), 2.57-2.17 (m, 16H), 2.17-2.01 (m, 2H), 1.90 (s, 4H), 1.76-1.54 (m, 8H), 1.53-1.29 (m, 12H), 1.06 (s, 9H), 0.96 (d, J=15.5 Hz, 3H) ppm. LC/MS (ESI) m/z 1709.8; [M+H]⁺ calcd for Cs₆H₁₁₃ClF₃N₁₂O₁₁S₄⁺: 1709.72.

### Example 66. Platelet Toxicity Assay

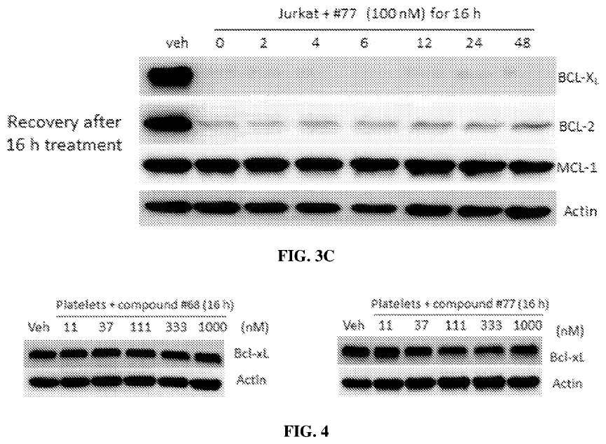

Platelet-rich plasma (PRP) was purchased from Life South Community Blood Centers (Gainesville, FL, USA). PRP was transferred into a 50 mL tube containing 5 mL of the acid citrate buffer (Cat. No. sc-214744, Santa Cruz Biotechnology). To prevent clotting, prostaglandin E1 (PGE1, Cat. No. sc-201223A, Santa Cruz Biotechnology, Dallas, TX, USA) and apyrase (Cat. No. A6237, Sigma-Aldrich, St. Louis, MO, USA) were added to final concentrations of 1 M and 0.2 U/mL, respectively. After gently mixing the solution, platelets were pelleted by centrifugation at 1200 g for 10 min without break. Pelleted platelets were gently washed without disturbing platelets using 2 mL of HEPES Tyrode's buffer (Cat. No. PY-921WB, Boston BioProducts, Ashland, MA, USA) containing 1 M PGE1 and 0.2 U/mL apyrase. After washing, pellets were gently suspended in 10 mL of HEPES Tyrode's buffer containing 1 M PGE1 and 0.2 U/mL apyrase. Then, the platelet number was counted using a HEMAVET 950FS hematology analyzer (Drew Scientific, Inc., Oxford, CT, USA). For viability assays, the platelet number was adjusted to 2×10⁸/mL in HEPES Tyrode's buffer containing 1 M PGE1 and 0.2 U/mL apyrase. Each treatment was given in 2 mL of the platelet suspension in 15 mL polypropylene tubes. The tubes were placed on a rotating platform at room temperature, and the viability of platelets was measured after 48 h treatment by using the MTS reagent (Cat. No. G1111, Promega, Madison, WI, USA). The data were analyzed by the GraphPad Prism 7 software for the calculation of IC₅₀ values. Representative data are presented in FIG. 4.

### Example 67. Cell Viability Assay

Acute lymphoblastic leukemia cells (MOLT-4 and RS4; 11) were incubated with increasing concentrations of Bcl-xL degraders for 48 h. Cell viability was measured by tetrazolium-based MTS assay. The IC₅₀ values of individual agents were calculated with GraphPad Prism and presented in Table 2. The IC₅₀ values of additional agents were calculated with GraphPad Prism and presented in Table 2A.

TABLE 2
Anti-cancer activity of compounds of Formulae (I) and (II).

| | | Anti-Cancer Activity IC₅₀ (48 hr)^{a} | |
|---|---|---|---|
| Compound # | MW | MOLT-4 | RS4; 11 |
| 1 | 1663.46 | +++ | ++ |
| 2 | 1691.51 | +++ | +++ |
| 3 | 1719.57 | ++++ | +++ |
| 4 | 1691.51 | +++ | +++ |
| 5 | 1719.57 | +++ | ++ |
| 6 | 1747.62 | + | ++ |
| 7 | 1720.56 | ++ | +++ |
| 8 | 1748.61 | +++ | +++ |
| 9 | 1776.66 | +++ | +++ |
| 10 | 1662.47 | ++ | ++ |
| 11 | 1690.53 | +++ | +++ |
| 12 | 1718.58 | ++++ | +++ |
| 13 | 1719.57 | ++ | ++ |
| 14 | 1747.62 | +++ | +++ |
| 15 | 1775.68 | +++ | +++ |
| 16 | 1640.51 | ++++ | +++ |
| 17 | 1668.52 | ++ | +++ |
| 18 | 1696.57 | +++ | +++ |
| 19 | 1724.63 | ++++ | +++ |
| 20 | 1612.41 | +++ | ++++ |
| 21 | 1600.36 | ++ | +++ |
| 22 | 1697.52 | ++ | ++ |
| 23 | 1685.46 | + | + |
| 25 | 1669.51 | ++++ | ++++ |
| 26 | 1668.52 | ++++ | +++ |
| 27 | 1668.52 | ++ | ++ |
| 28 | 1655.52 | ++ | +++ |
| 29 | 1669.55 | ++ | +++ |
| 30 | 1683.57 | ++ | +++ |
| 31 | 1681.51 | ++ | ++ |
| 32 | 1695.55 | ++++ | ++++ |
| 33 | 1554.43 | +++ | ++++ |
| 34 | 1512.40 | ++++ | ++++ |
| 35 | 1538.44 | +++ | + ++ |
| 36 | 1552.4 | +++ | ++ ++ |
| 37 | 1524.4 | ++++ | + + |
| 38 | 1538.44 | ++++ | + ++ |
| 39 | 1552.46 | ++++ | ++++ |
| 40 | 1552.46 | ++++ | ++++ |
| 41 | 1542.42 | +++ | ++++ |
| 42 | 1542.42 | +++ | ++++ |
| 43 | 1556.45 | +++ | ++++ |
| 44 | 1568.46 | ++ | +++ |
| 45 | 1582.49 | ++ | +++ |
| 46 | 1568.46 | +++ | ++++ |
| 47 | 1582.49 | ++ | +++ |
| 48 | 1595.49 | +++ | ++++ |
| 49 | 1485.33 | + | +++ |
| 50 | 1499.35 | + | +++ |
| 52 | 1566.45 | ++++ | ++++ |
| 53 | 1564.47 | +++ | ++++ |
| 54 | 1620.54 | ++++ | ++++ |
| 55 | 1568.46 | ++++ | ++++ |
| 56 | 1566.49 | ++++ | ++++ |
| 57 | 1601.49 | ++ | +++ |
| 58 | 1639.52 | ++++ | ++++ |
| 59 | 1655.52 | ++++ | ++++ |
| 60 | 1653.55 | ++++ | ++++ |
| 61 | 1599.46 | ++++ | ++++ |
| 62 | 1629.48 | ++++ | ++++ |
| 63 | 1643.51 | +++ | ++++ |
| 64 | 1653.50 | ++++ | ++++ |
| 65 | 1653.50 | ++++ | ++++ |
| 66 | 1653.50 | ++++ | ++++ |
| 67 | 1651.53 | ++++ | ++++ |
| 68 | 1681.52 | ++++ | ++++ |
| 69 | 1707.60 | ++++ | ++++ |
| 70 | 1735.65 | ++++ | ++++ |
| 71 | 1688.55 | ++ | +++ |
| 72 | 1731.58 | ++++ | ++++ |
| 73 | 1745.61 | ++++ | ++++ |
| 74 | 1759.63 | ++++ | +++ |
| 75 | 1682.55 | +++ | +++ |
| 76 | 1682.55 | ++++ | ++++ |
| 77 | 163.53 | ++++ | ++++ |
| 78 | 1734.58 | ++++ | +++ |
| 79 | 1734.58 | ++++ | ++++ |
| 80 | 1747.62 | ++++ | ++++ |
| 81 | 1669.55 | ++++ | ++++ |
| 82 | 1669.55 | ++++ | +++ |
| 83 | 1683.57 | +++ | ++ |
| 84 | 1709.61 | ND | ND |
| 85 | 1654.54 | ND | ND |
| 86 | 1682.59 | ND | ND |
| 87 | 1647.45 | ND | ND |
| 88 | 1675.50 | + | + |
| 89 | 1655.52 | +++ | ++++ |
| 90 | 1655.52 | ND | ND |
| 91 | 1655.52 | ++++ | ++++ |
| 92 | 1667.53 | + | +++ |
| 93 | 1681.56 | + | + |
| 94 | 1681.56 | ND | ND |
| 95 | 1667.53 | ++++ | ++++ |
| 96 | 1691.57 | ++++ | ++++ |
| 97 | 1706.58 | ND | ND |
| 98 | 1704.57 | ND | ND |
| 99 | 1718.59 | ND | ND |
| 100 | 1732.58 | ++ | +++ |
| 101 | 1746.60 | ND | ND |
| 102 | 1746.64 | ND | ND |
| 103 | 1662.52 | ND | ND |
| 104 | 1690.57 | ND | ND |
| 105 | 1681.52 | ++++ | ++++ |

^{a}<20 nM = ++++, 20-100 nM = +++, 100-500 nM = ++, 0.5-10 μM = +, ND = Not Determined

TABLE 2A
Anti-cancer activity of additional compounds of Formula (I).

| | | Anti-Cancer Activity IC₅₀ (48 hr)^{a} | |
|---|---|---|---|
| Compound # | MW | MOLT-4 | RS4; 11 |
| 109 | 1635.46 | ++ | +++ |
| 110 | 1663.51 | +++ | ++++ |
| 111 | 1736.55 | ++++ | ++++ |
| 112 | 1749.60 | +++ | ++++ |
| 113 | 1761.60 | +++ | ++++ |
| 114 | 1803.69 | ++++ | ++++ |
| 115 | 1711.54 | ++++ | ++++ |
| 116 | 1709.61 | ++++ | ++++ |
| 117 | 1724.63 | +++ | ++++ |
| 118 | 1710.60 | ++++ | ++++ |
| 119 | 1710.60 | ++++ | ++++ |
| 120 | 1710.60 | ++++ | ++++ |

^{a}<20 nM = ++++, 20-100 nM = +++, 100-500 nM = ++.

### Example 68: Protein Degradation Assays in Jurkat Cells and Human Platelets

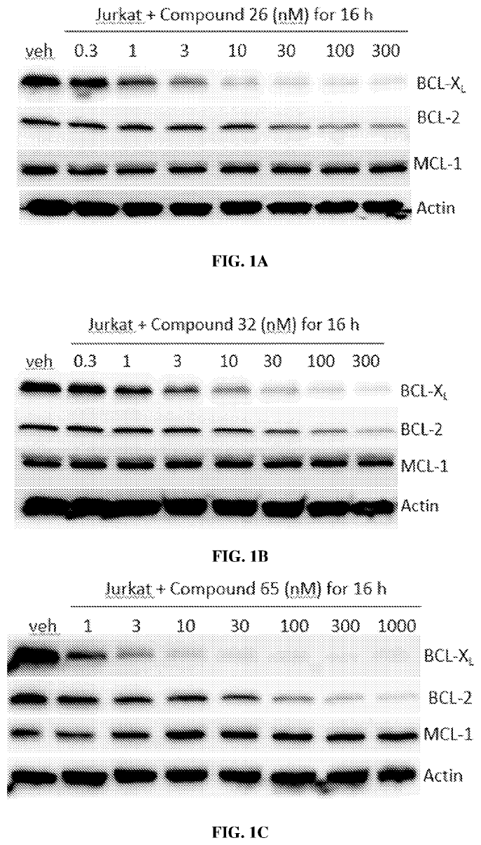

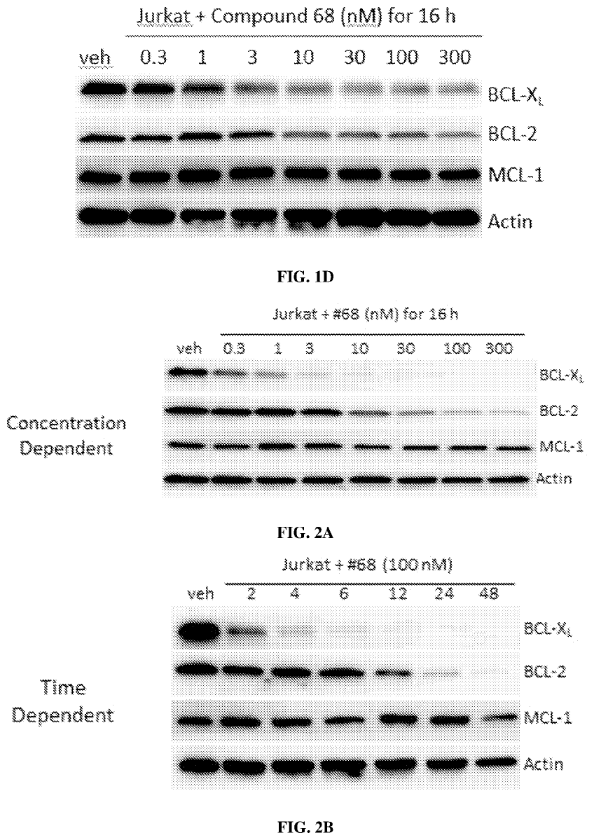

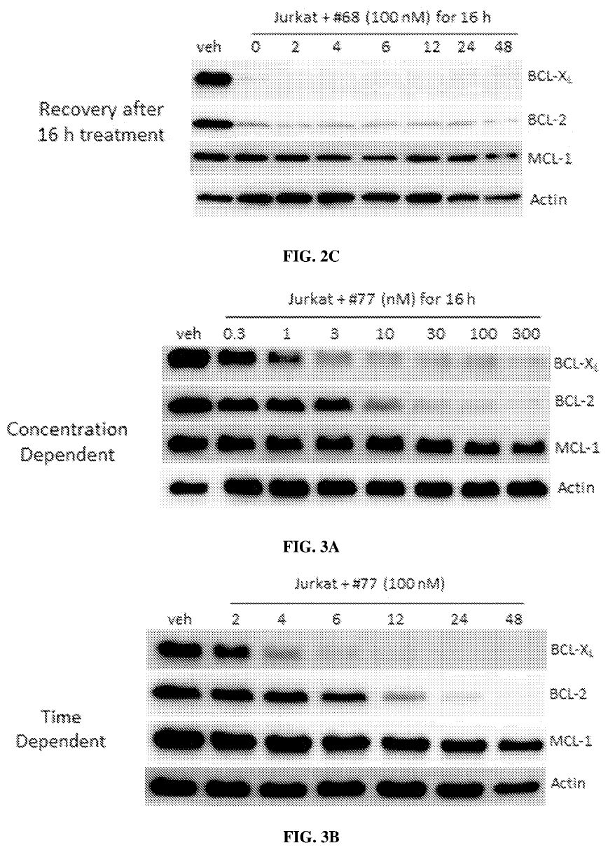

Jurkat cells and human platelets were incubated with increasing concentrations of test compounds for 16 h. The cells were harvested and lysed in RIPA lysis buffer supplemented with protease and phosphatase inhibitor cocktails. An equal amount of protein (20 pg/lane) was resolved on a pre-cast 4-20% SDS-PAGE gel. Proteins were subsequently transferred to NOVEX PVDF membranes by electrophoresis. The membranes were blocked in blocking buffer (5% non-fat dry milk in TBS-T), and incubated with primary antibodies (at optimized concentrations) overnight at 4° C. After three washings in TBS-T, the membranes were incubated with an appropriate FIRP-conjugated secondary antibody for 1 h at room temperature. After extensive washing for three times, the proteins of interest was detected with ECL western blotting detection reagents and recorded with autoradiography (Pierce Biotech, Rockford, IL, USA). The primary antibodies for Bcl-xL (Cat #2762), Bcl-2 (Cat #2872), Mcl-1 (Cat #5453) and j-actin (Cat #4970) were purchased from Cell Signaling Technology. The relative band intensity was measured using ImageJ software and normalized to β-actin. Representative data are presented in FIGS. 1A to 3C.

### Example 69: Ternary Complex Assay

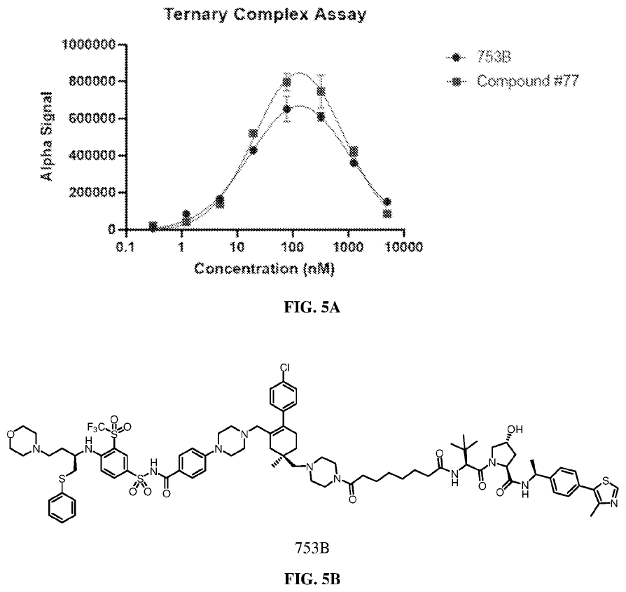

To detect ternary complex formation induced by the compounds, AIphaLISA assay was used to measure luminescence signals arisen from proximity of Bcl-xL bounded acceptor beads and VHL- or CRBN—bounded donor beads. Briefly, to a 96-well PCR plate, 10 μL of 20 nM 6-His tagged Bcl-xL protein can be mixed with 10 μL of 20 nM GST-tagged VHL complex protein and 10 μL of serially diluted testing compounds. After incubating at room temperature for 30 14 min, 5 μL of 160 μg/mL Glutathione donor beads (PerkinEImer) was added and the mixture was incubated at dark for 15 min. 5 μL of 160 μg/mL of anti-His acceptor beads was added lastly and the mixture was incubated for an additional 45 min before being transferred to two adjacent wells (17 μL each) of 384-well white OptiPIate (PerkinEImer). The luminescence signals were detected on a Biotek's Synergy Neo2 multi-mode plate reader installed with an AphaScreen filter cube. All reagents were diluted in an assay buffer of 25 mM HEPES, pH 7.5, 100 mM NaCl, 0.1% BSA, and 0.005% tween 20 prior incubation. Representative data are presented in FIGS. 5A and 5B.

### Example 70. Additional Data

The inhibitory activity against MOLT-4 cells and platelets was determined for exemplary compounds of the present disclosure (Table 3 to Table 6).

Compound ABT-263 has the structure:

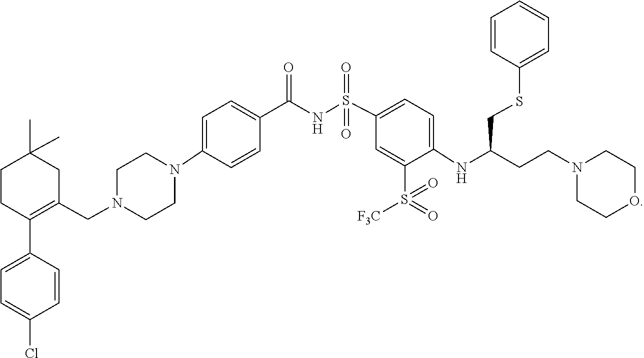 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

Compound DT2216 has the structure:

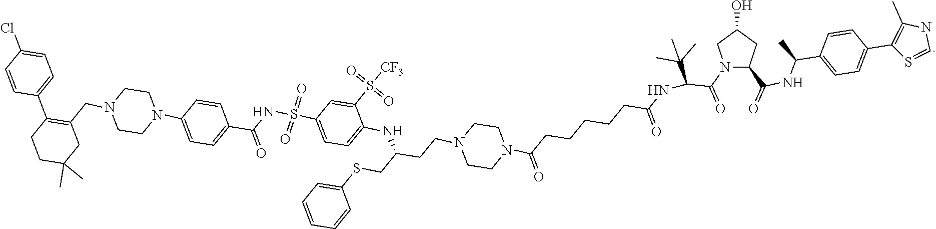

Compound PZ753B has the structure:

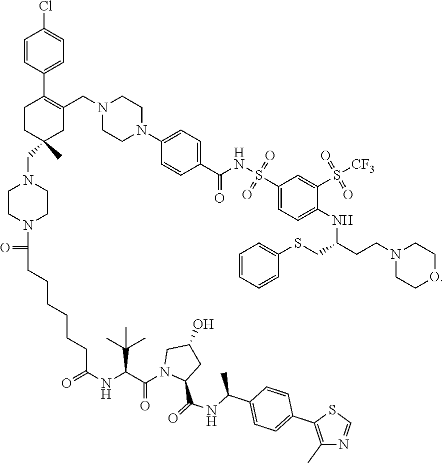 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

Compound PP18319C8 has the structure:

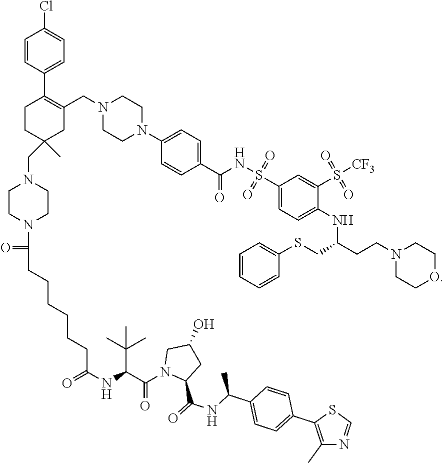 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

TABLE 3
IC₅₀ of representative compounds against
MOLT-4 and human platelets.

| | Platelets | MOLT-4 | Platelets/MOLT-4 |
|---|---|---|---|
| Compound | 48 h IC₅₀ (μM) | 48 h IC₅₀ (nM) | IC₅₀ Fold change |
| ABT-263 | 0.312 | 220 | 1.4 |
| 12 | 2.16 | 17.5 | 123 |
| 38 | 3.0 | 17.6 | 170 |
| 39 | 4.57 | 14.3 | 319 |
| 41 | 4.9 | 38.1 | 128 |
| 66 | 0.853 | 2.3 | 370 |
| 68 | 0.777 | 3.2 | 240 |

IC₅₀ values are reported as the mean deviation from two independent experiments performed in triplicates. Cells were treated for 48 h.

TABLE 4
IC₅₀ of representative compounds against
MOLT-4, RS4, and human platelets.

| | MOLT-4 | RS4 | Platelets | PLT/MOLT-4 |
|---|---|---|---|---|
| | 48 h IC₅₀ | 48 h IC₅₀ | 48 h IC₅₀ | IC₅₀ Fold |
| Compound | (nM) | (nM) | (nM) | Change |
| ABT-263 | 220 | 38 | 260 | — |
| DT2216 | 50-80 | 180-230 | >10,000 | >150 |
| PZ753B | 6.9 | 5.8 | 1,050 | 152 |
| PP18319C8 | 17.4 | 18.7 | 1,640 | 94 |
| 66 | 2.3 | 1.6 | 853 | 370 |
| 59 | 0.7 | 1.6 | ND | — |
| 55 | 11 | 3.2 | ND | — |
| 105 | 4 | 3 | ND | — |
| | | | (777) | |
| 62 | 21 | <7 | ND | — |
| | | | (>10,000) | |
| 3 | 9.6 | 44.9 | 4,900 | 510 |
| 72 | 4.4 | 7 | ND | — |
| 73 | 7.1 | 13.9 | ND | — |

ND = Not Determined

TABLE 5
IC₅₀ of representative compounds against
MOLT-4, RS4, and human platelets.

| | MOLT-4 | RS4 | Platelets |
|---|---|---|---|
| Compound | 48 h IC₅₀ (nM) | 48 h IC₅₀ (nM) | 48 h IC₅₀ (nM) |
| 16 | 12.7 | 42.8 | ND |
| 69 | 1.9 | 3.7 | 157 |
| 39 | 14.3 | 8.4 | 4,570 |
| 37 | 20 | 7 | 4,620 |
| PP18348C8 | 42.3 | 9.0 | ND |
| PZ18812B | 13.4 | 6.4 | ND |
| 46 | 55.9 | 5.8 | ND |
| 43 | 78.5 | 7.3 | ND |
| 52 | 15.2 | 2.8 | ND |
| 20 | 88.1 | 15.6 | ND |

ND = Not Determined

TABLE 6
IC₅₀ of representative compounds against
MOLT-4, RS4 and human platelets.

| | Platelets | MOLT-4 | Platelets/ | |
|---|---|---|---|---|
| | 4 th time | 48 h | MOLT-4 | RS4 |
| | 48 h IC₅₀ | IC₅₀ | IC₅₀ | 48 h IC₅₀ |
| Compound | (μM) | (nM) | Fold Change | (nM) |
| ABT-263 | 0.223 | 220 | 1 | 38 |
| 64 | 0.884 | 2.9 | 455 | 4.7 |
| 65 | 1.597 | 2.8 | 711 | 4.2 |
| 66 | 0.853 | 2.3 | 370 | 1.6 |
| 68 | 0.777 | 3.2 | 240 | 5.0 |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments provided herein provided herein. Such equivalents are intended with be encompassed by the following claims.

### EMBODIMENTS LISTING

1. A compound of Formula (I), or a pharmaceutically acceptable salt, hydrate, solvate, or prodrug thereof:

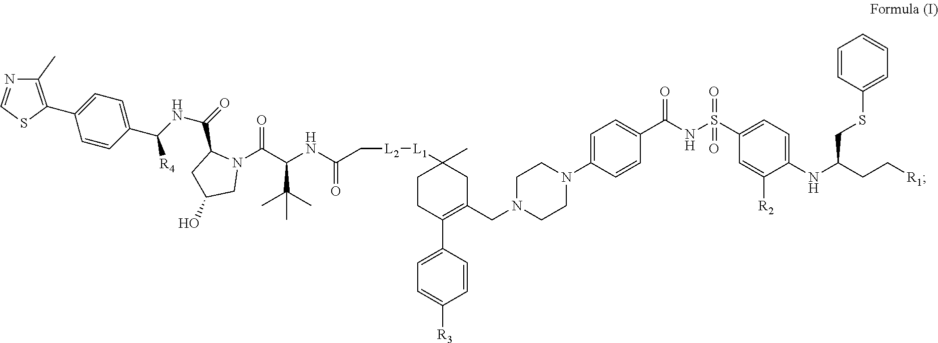

wherein R₁ is

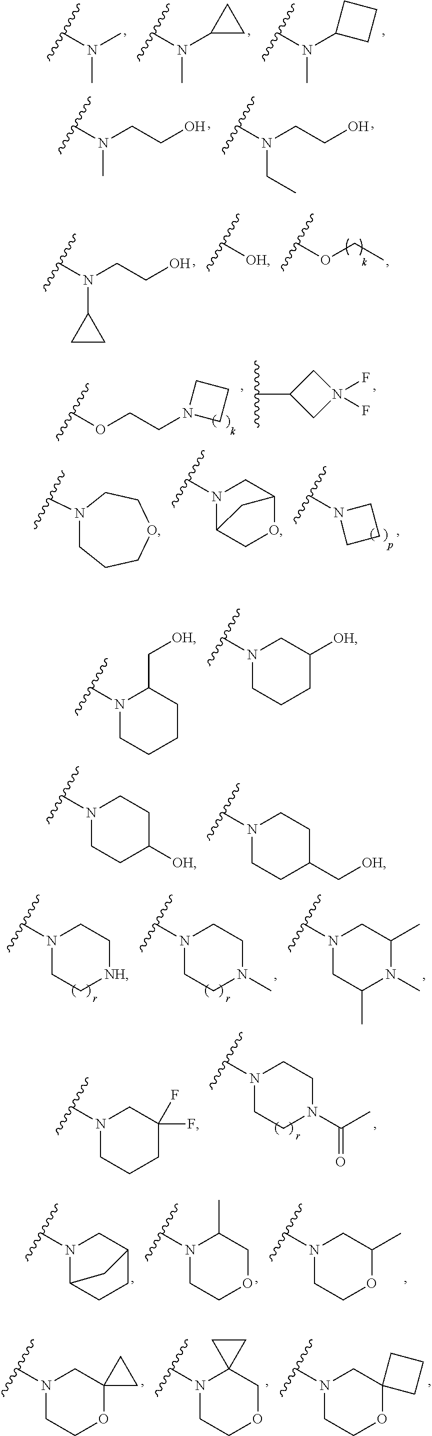

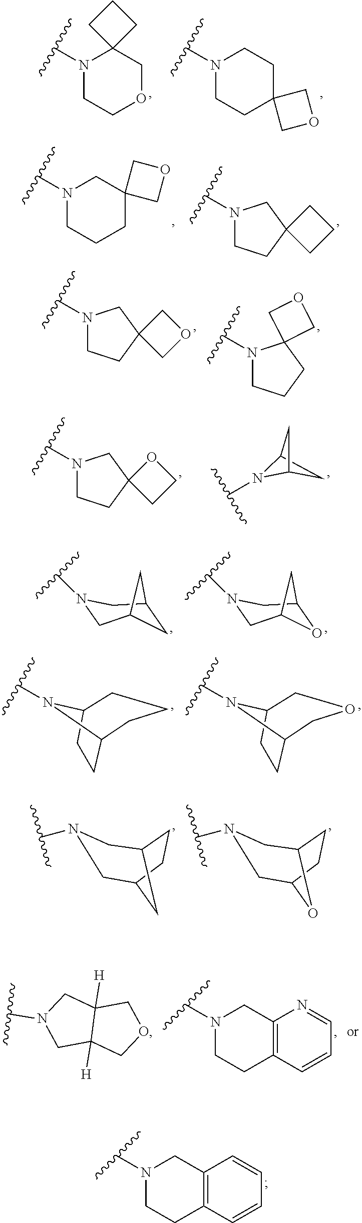

R₂ is NO₂, SO₂CF₃, or SO₂CF₂Cl;
R₃ is Cl, F, CF₂H, CFH₂ or CF₃;
R₄ is H or CH₃;
L₁ is

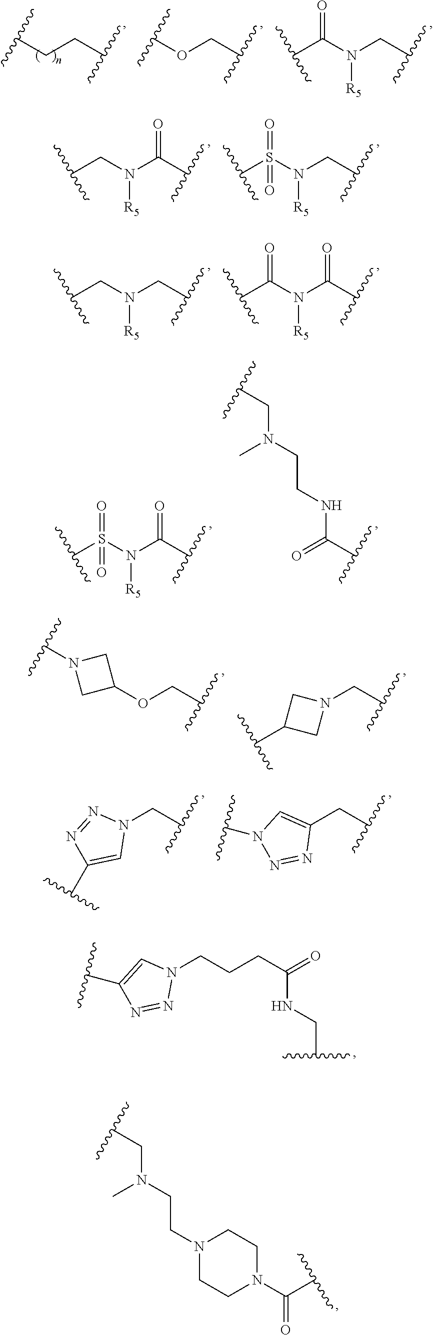

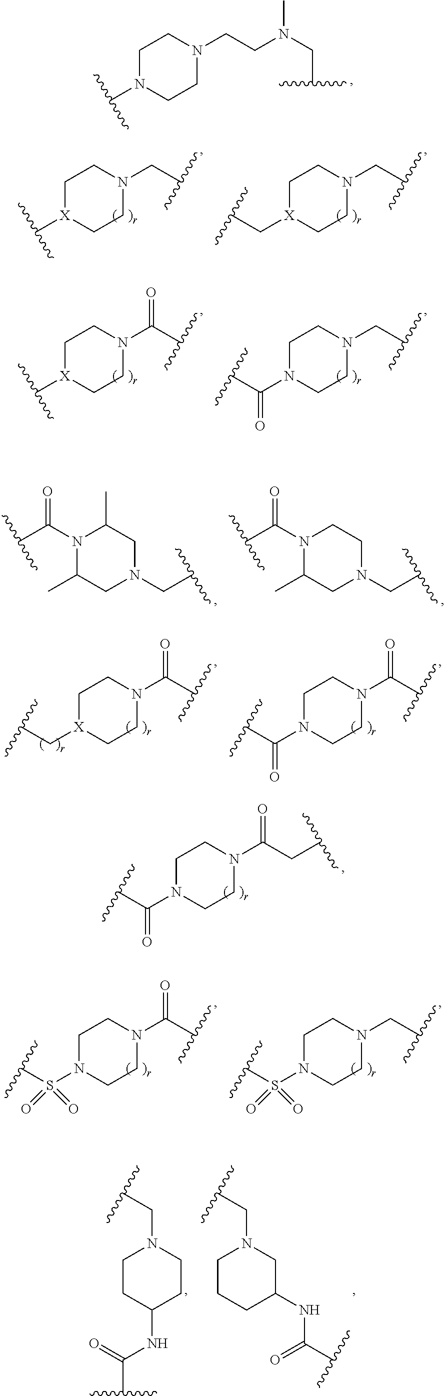

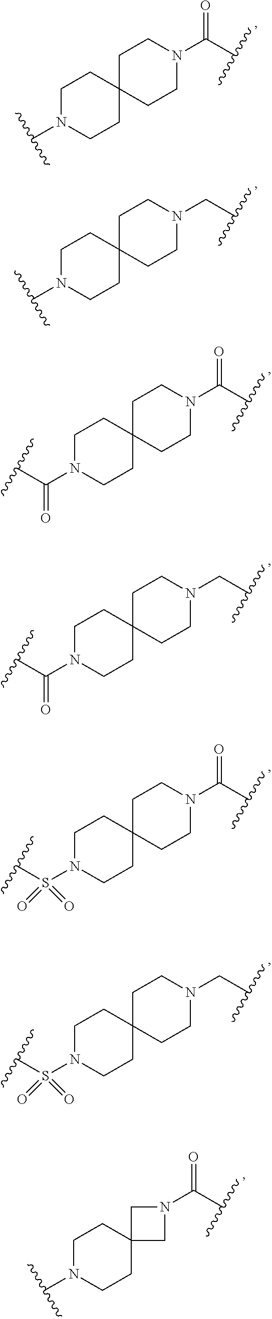

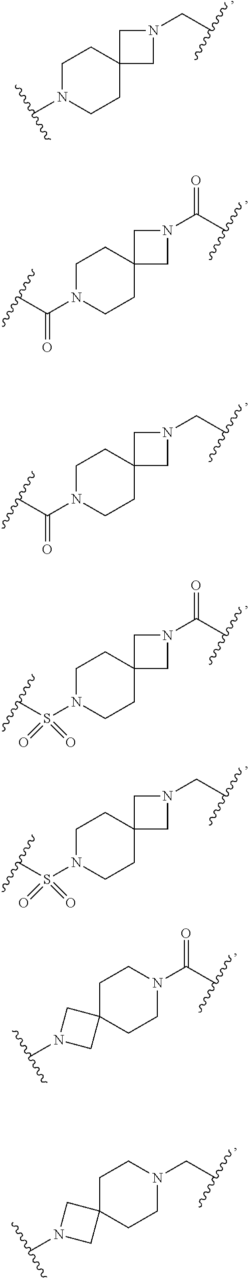

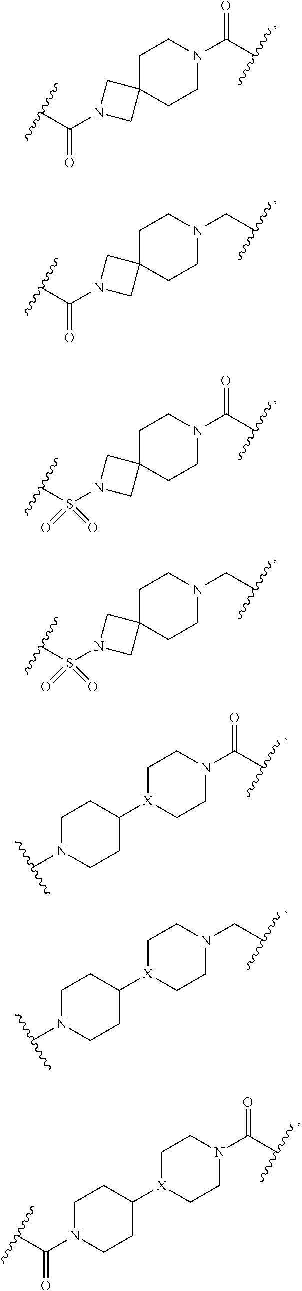

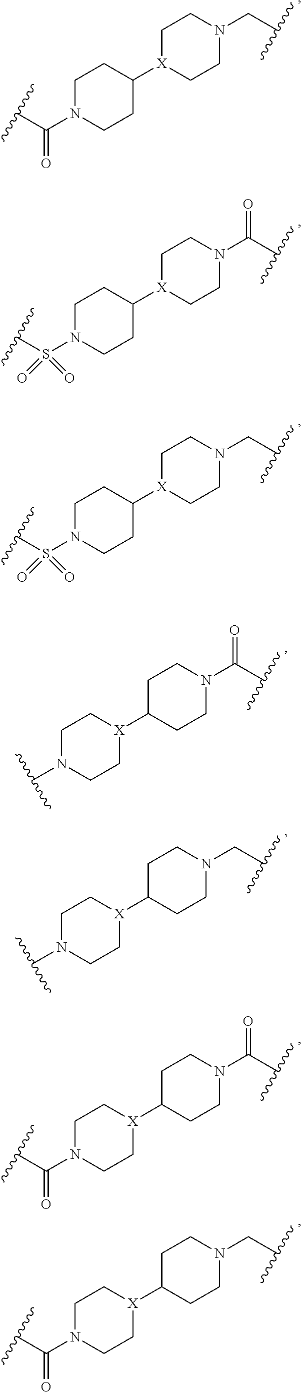

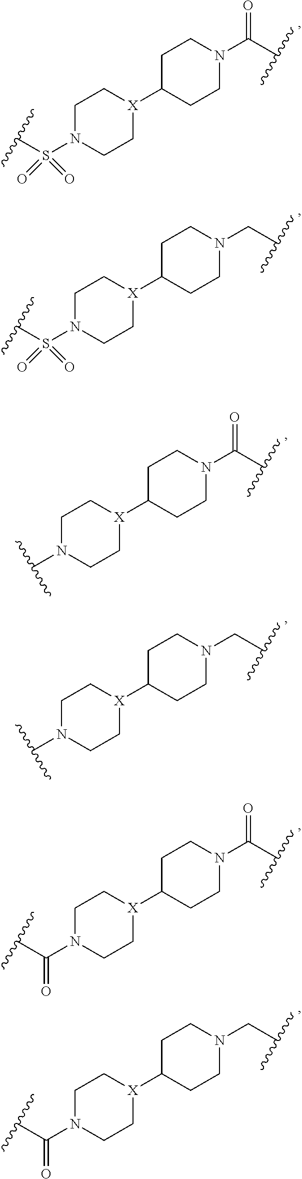

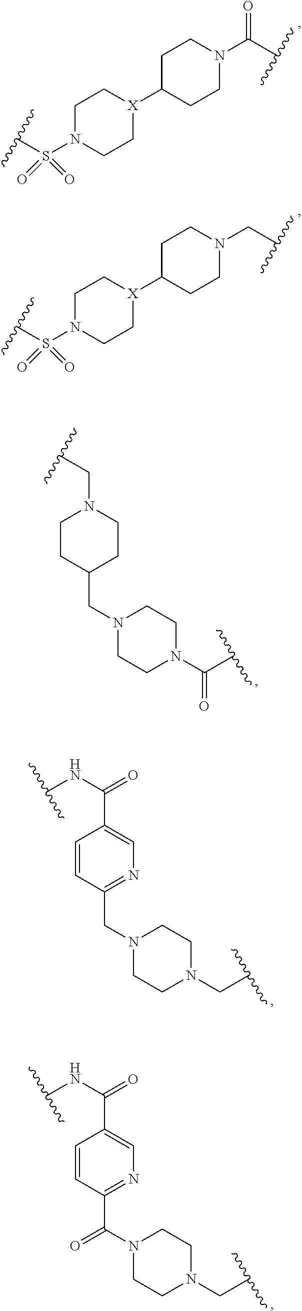

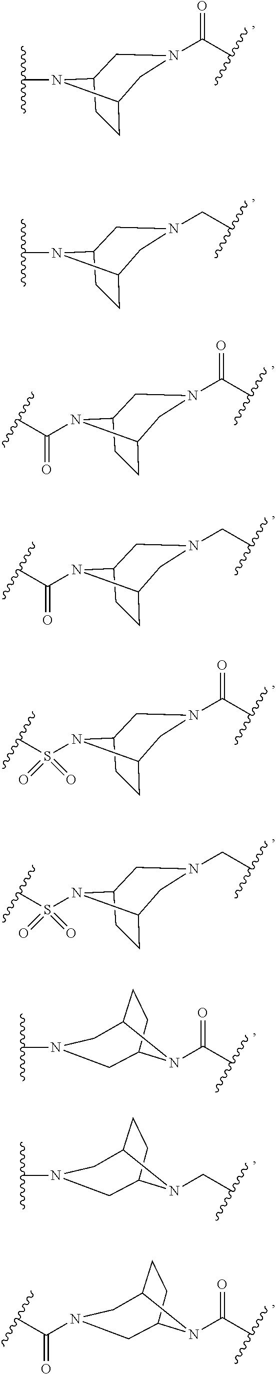

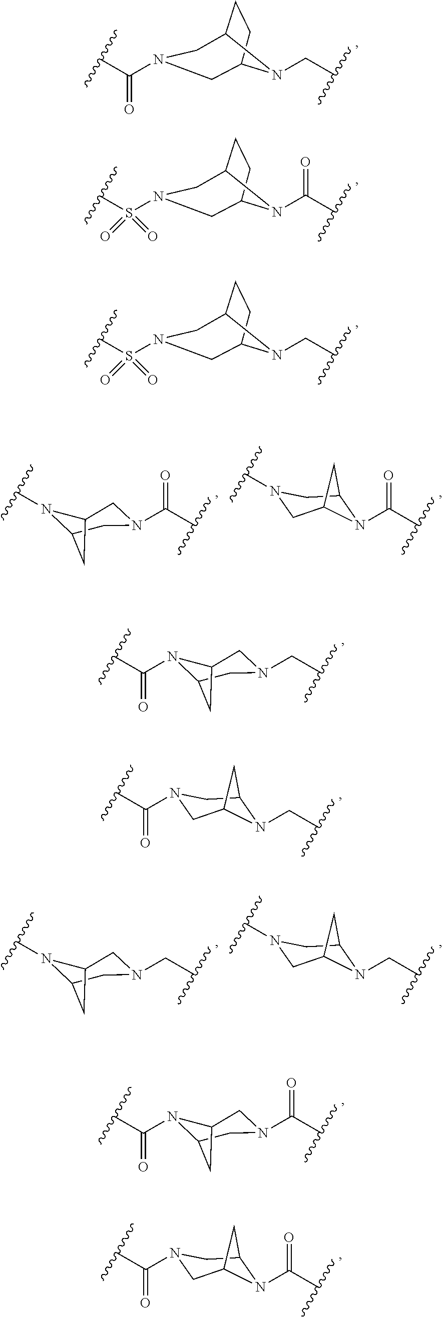

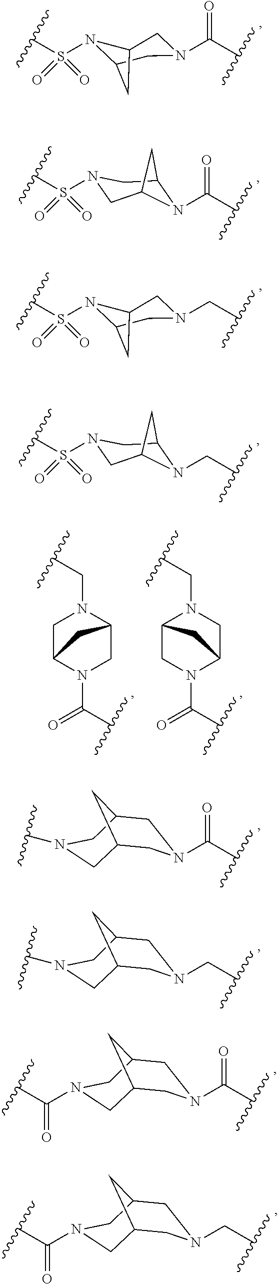

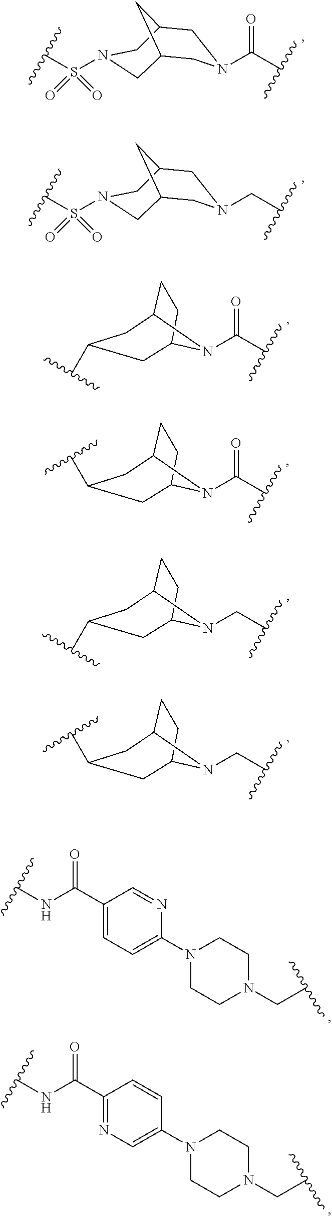

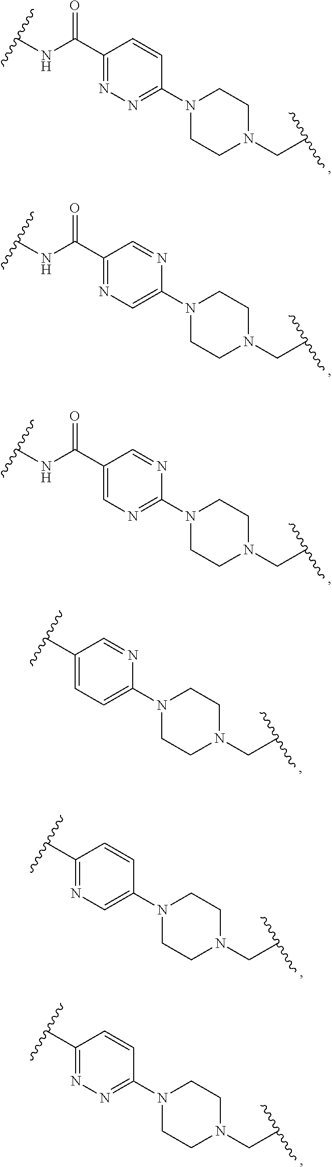

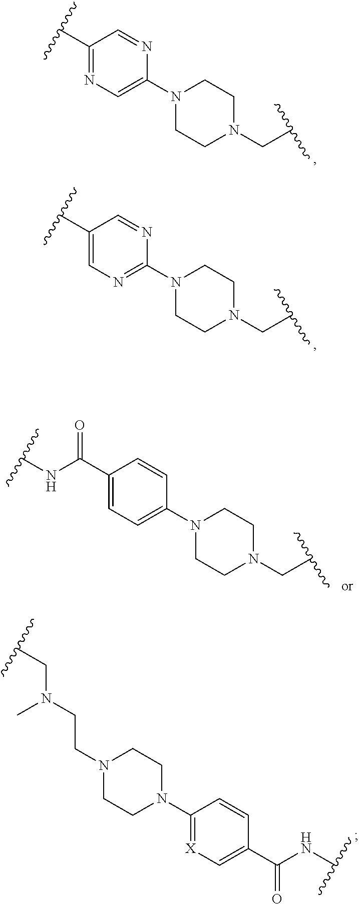

L₂ is a bond,

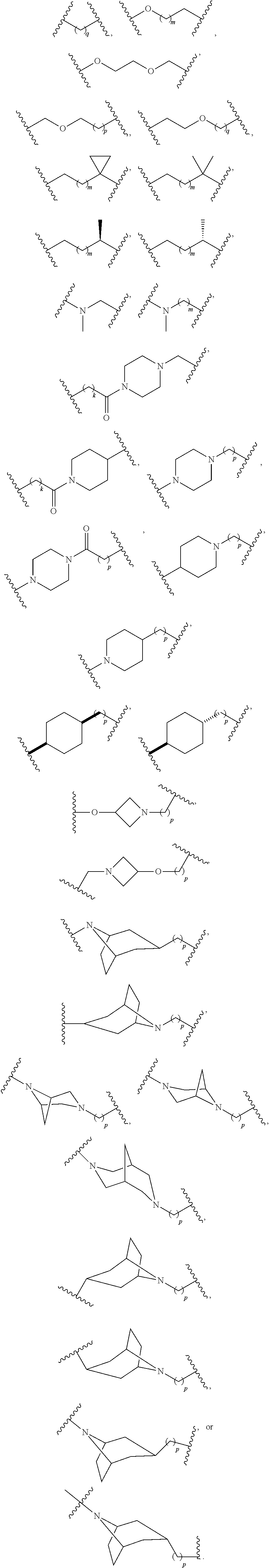

each X is independently CH or N
each k is independently 0, 1, 2, 3, 4, 5, or 6;
each m is independently 2, 3, 4, 5, 6, or 7;
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl.

2. The compound of embodiment 1, or a pharmaceutically acceptable salt, hydrate, solvate, or prodrug thereof, wherein:
L₁ is

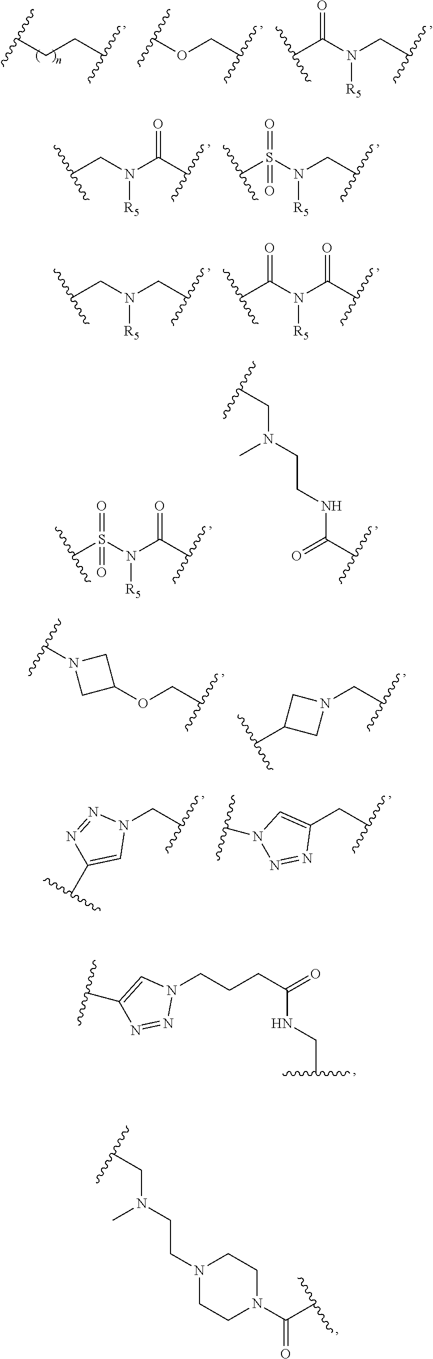

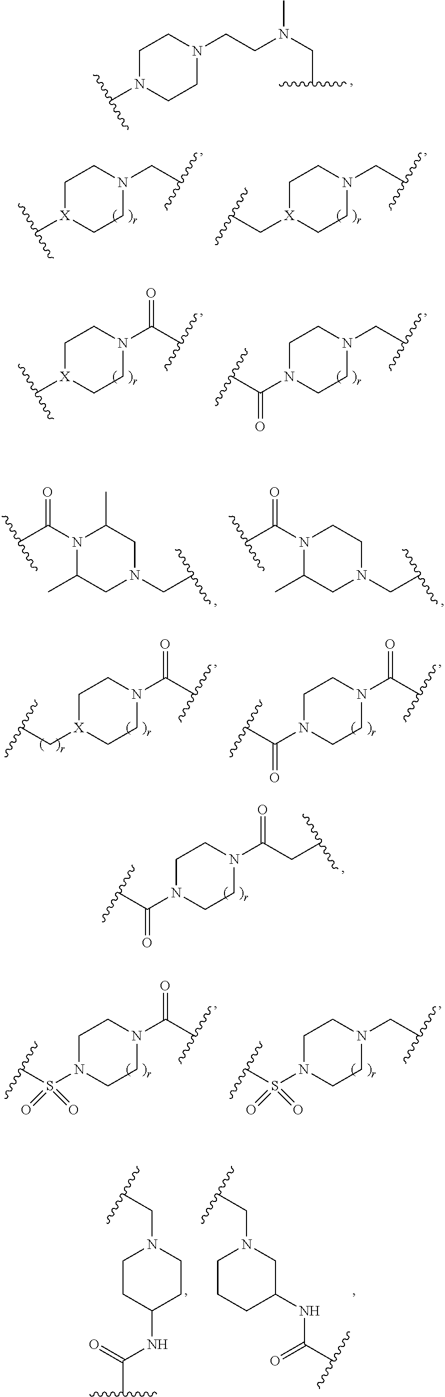

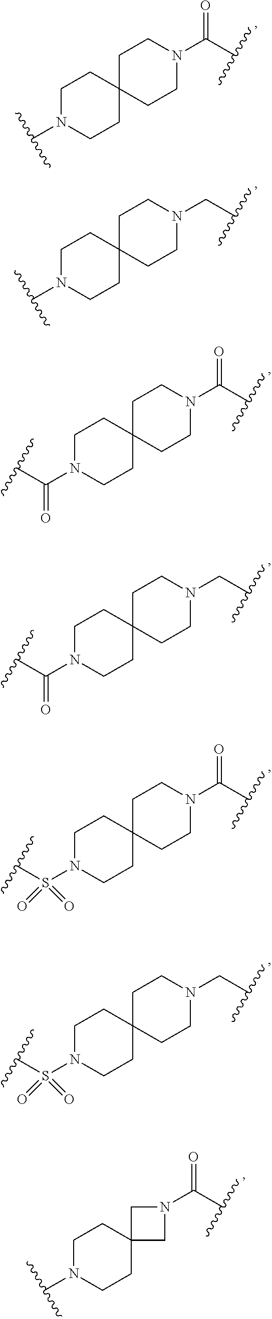

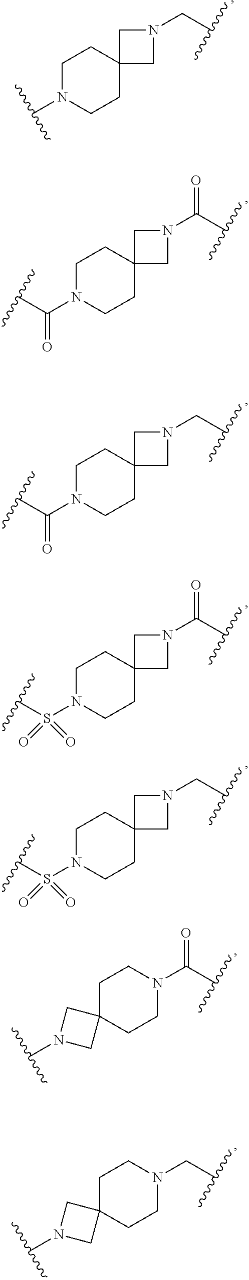

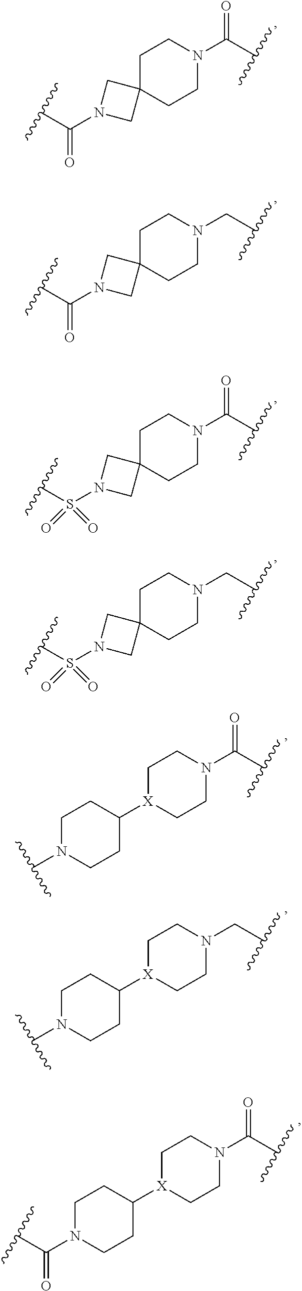

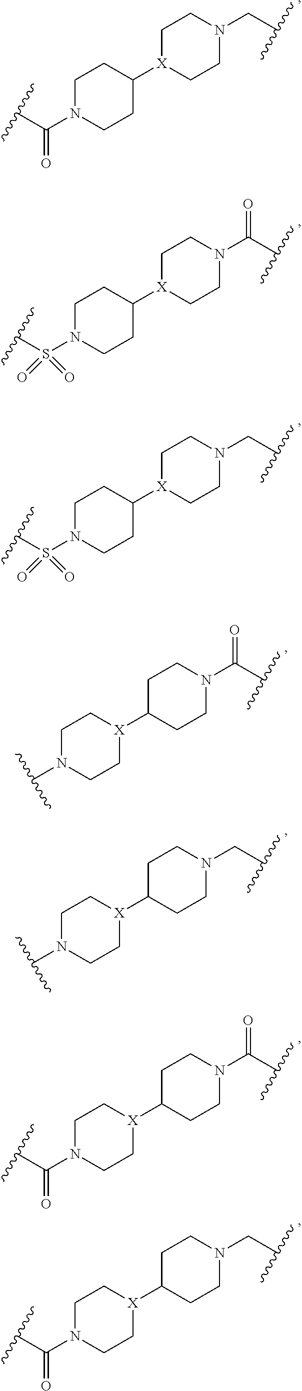

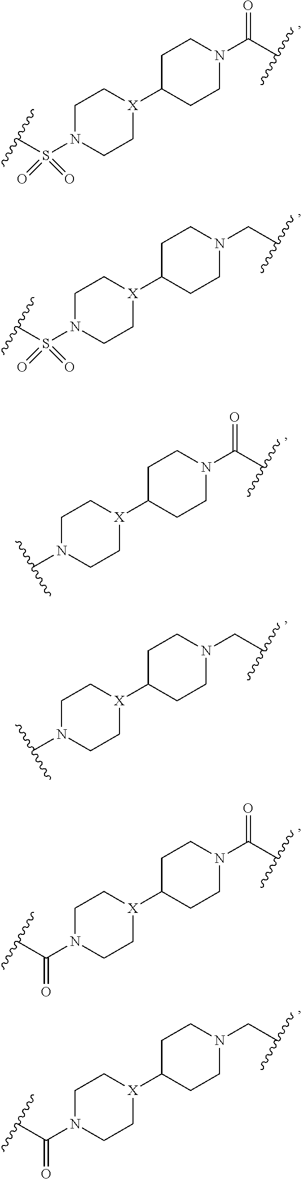

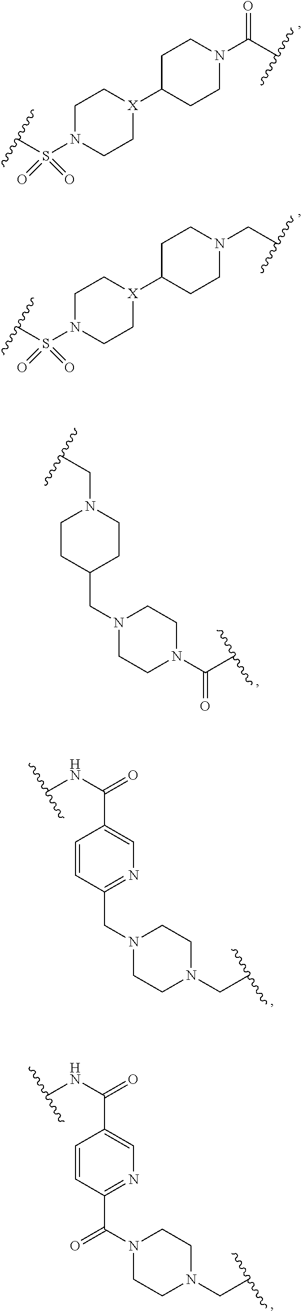

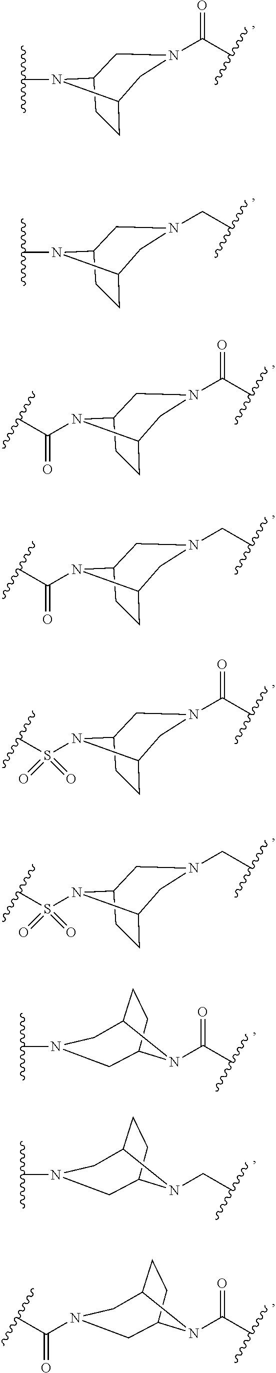

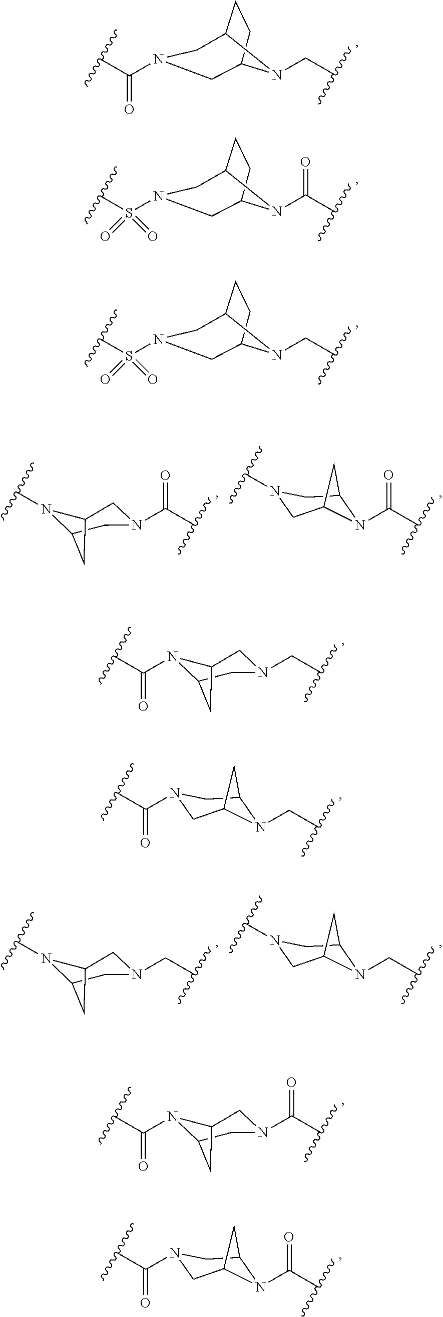

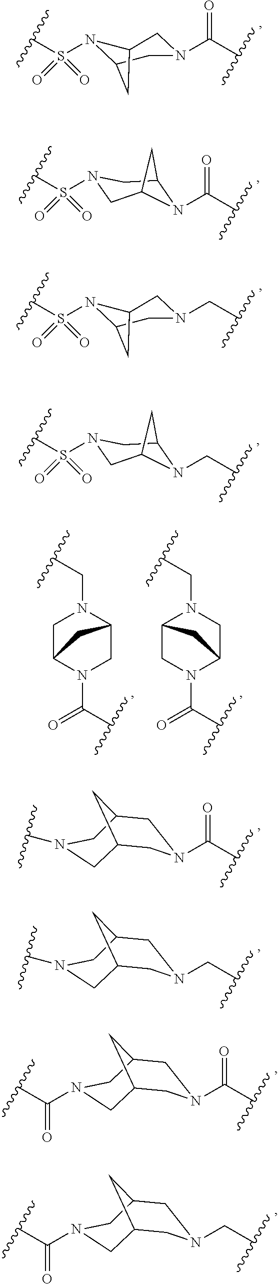

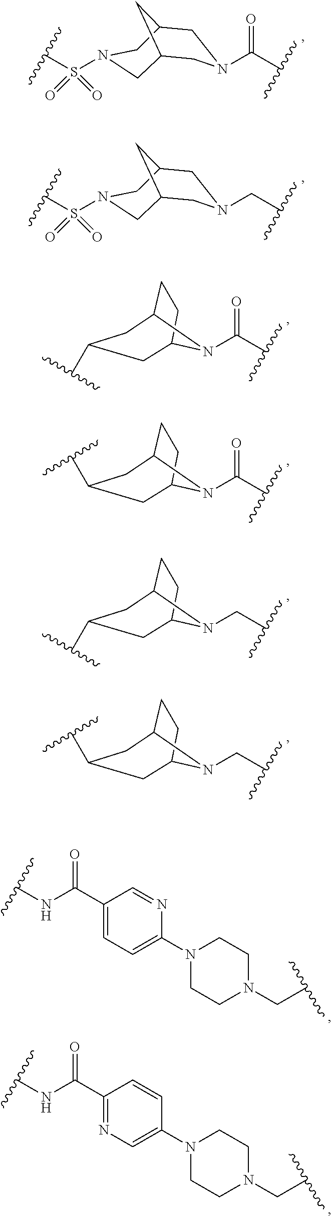

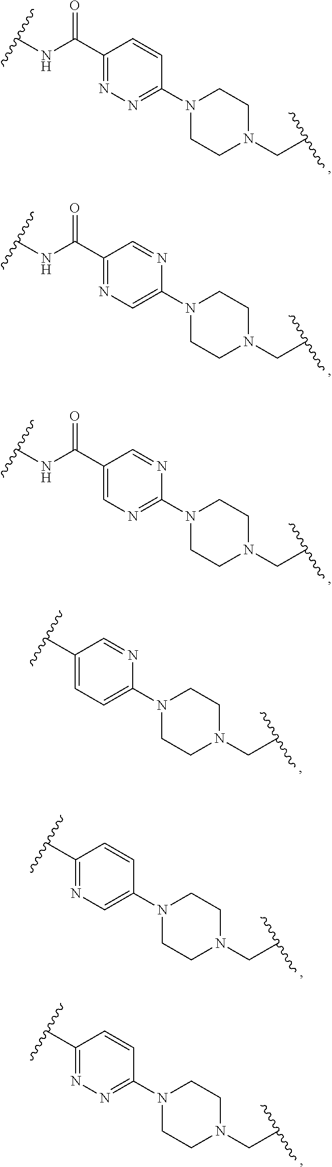

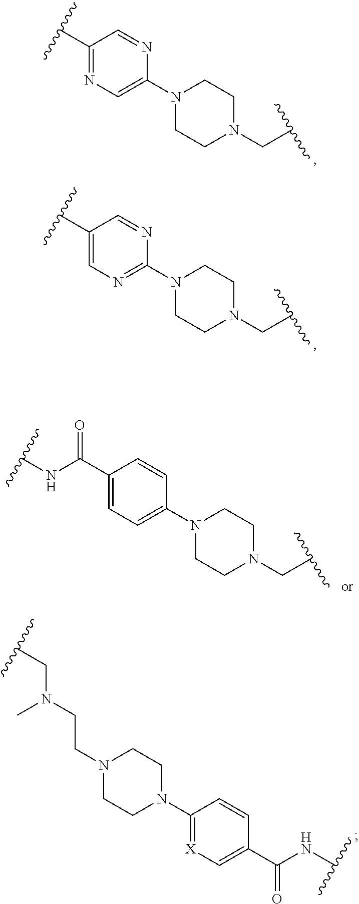

and
L₂ is a bond,

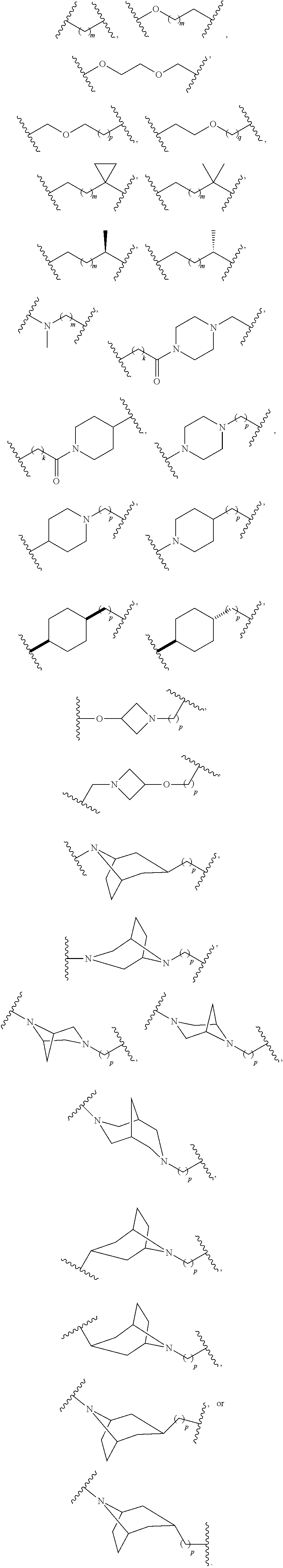

3. The compound of embodiment 1 or 2, or a pharmaceutically acceptable salt, hydrate, solvate, or prodrug thereof, wherein the compound is of formula:

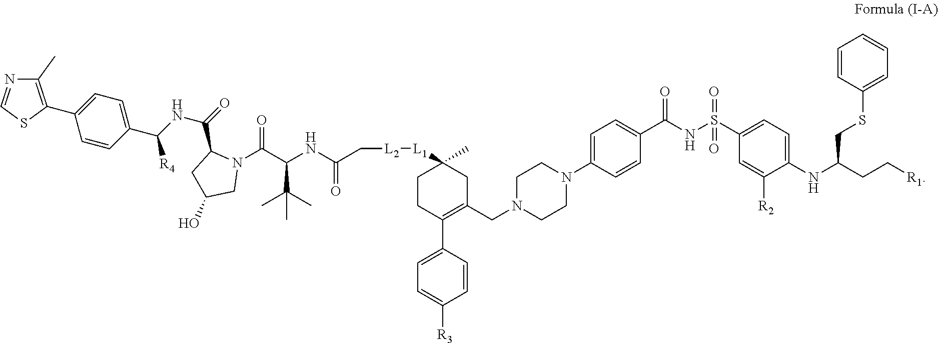

4. The compound of any one of embodiments 1-3, or a pharmaceutically acceptable salt, hydrate, solvate, or prodrug thereof, wherein L₁ is

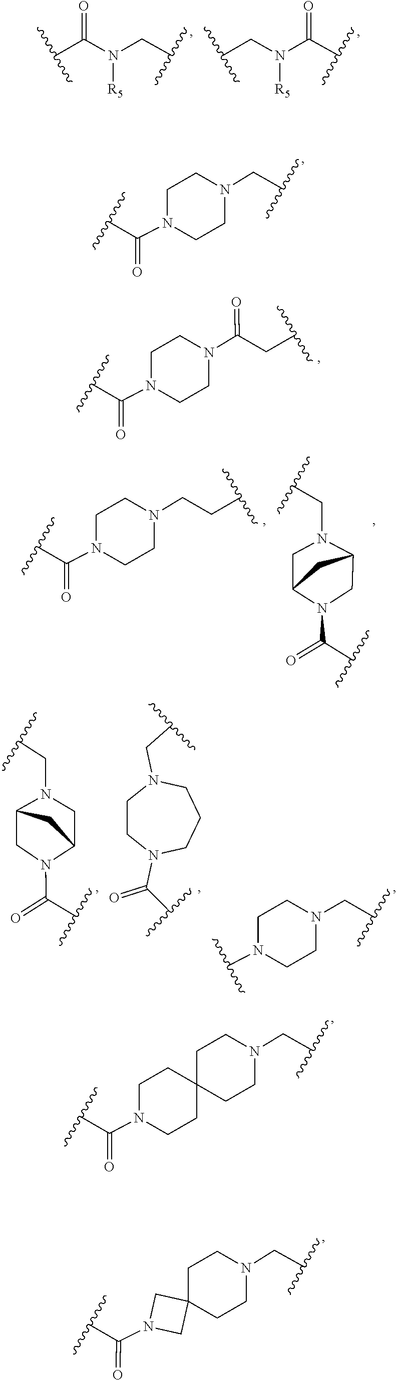

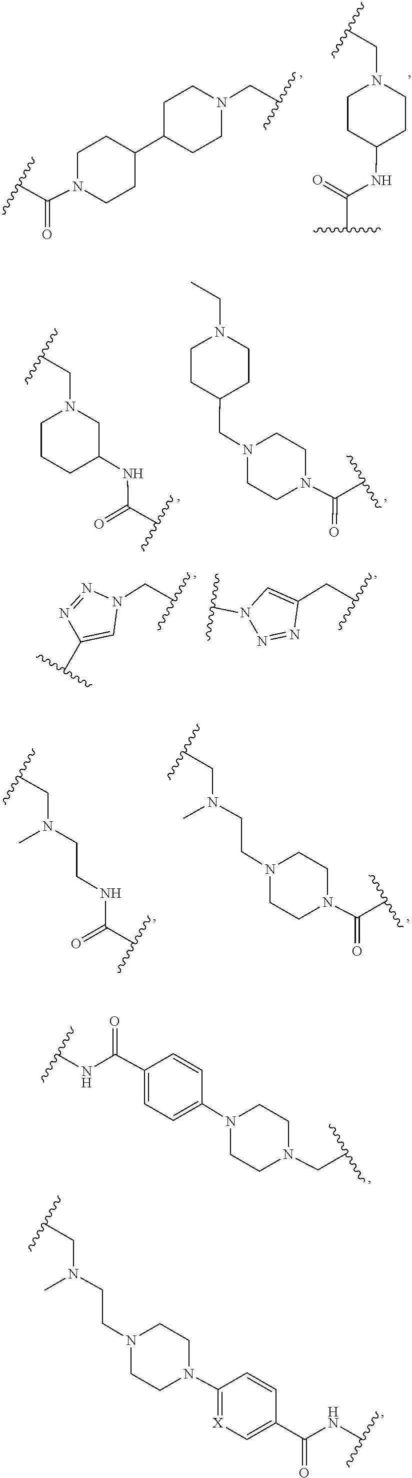

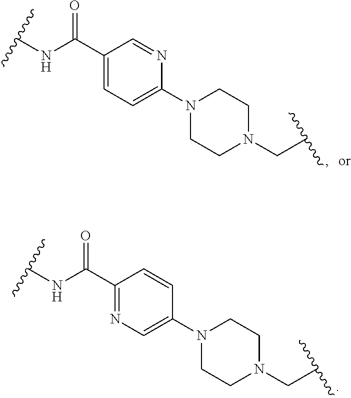 [SMILES: *CN1CCN(c2ccc(C(=O)N*)cn2)CC1 ; *CN1CCN(c2ccc(C(=O)N*)nc2)CC1]

5. The compound of embodiment 4, or a pharmaceutically acceptable salt, hydrate, solvate, or prodrug thereof, wherein L₁ is

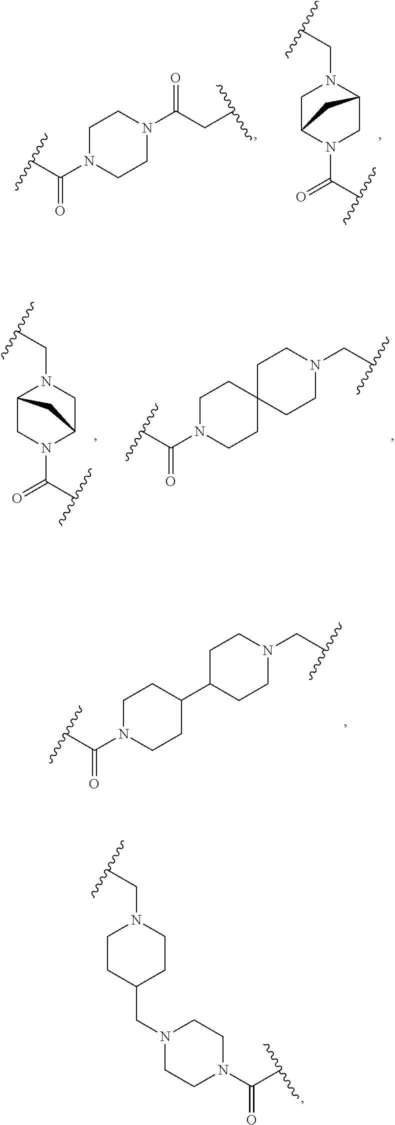 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2 ; *CN1CCC(C2CCN(C(*)=O)CC2)CC1 ; *CN1CCC(CN2CCN(C(*)=O)CC2)CC1]

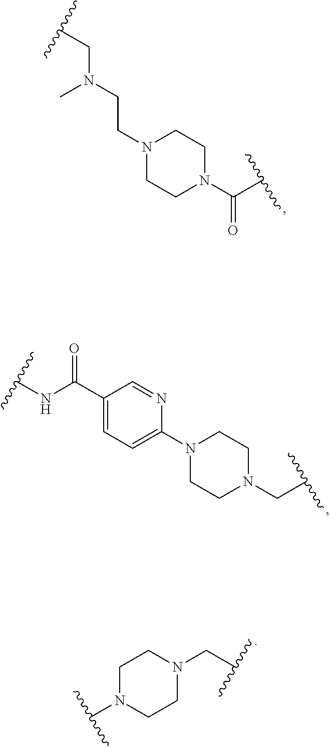 [SMILES: *CN(C)CCN1CCN(C(*)=O)CC1 ; *CN1CCN(c2ccc(C(=O)N*)cn2)CC1 ; *CN1CCN(*)CC1]

6. The compound of any one of embodiments 1-3, or a pharmaceutically acceptable salt, hydrate, solvate, or prodrug thereof, wherein L₁ is

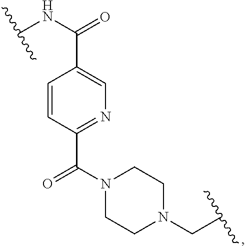 [SMILES: *CN1CCN(C(=O)c2ccc(C(=O)N*)cn2)CC1]

7. The compound of any one of embodiments 1-6, or a pharmaceutically acceptable salt, hydrate, solvate, or prodrug thereof, wherein L₂ is

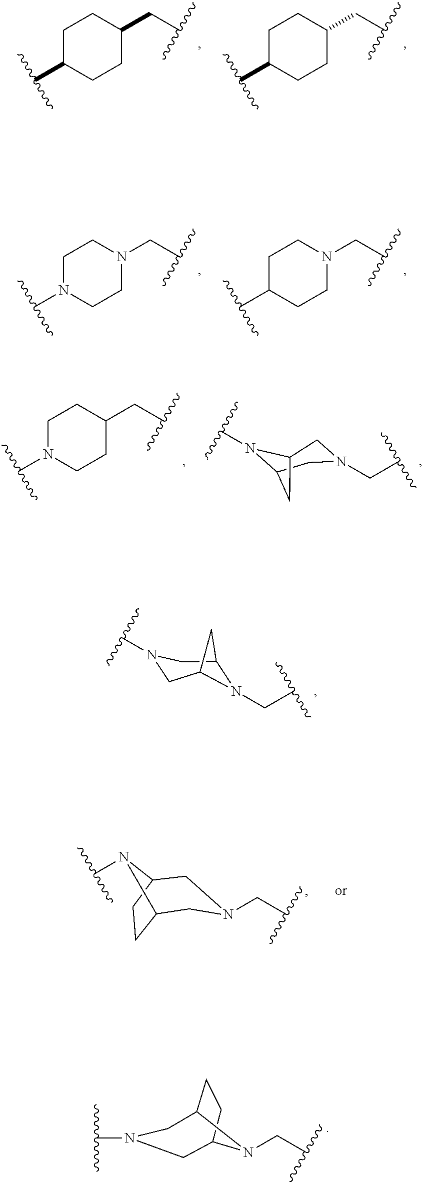

8. The compound of embodiment 7, or a pharmaceutically acceptable salt, hydrate, solvate, or prodrug thereof, wherein L₂ is

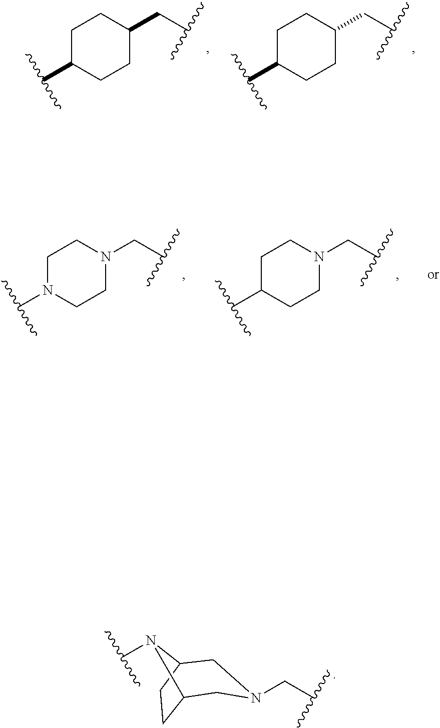 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

9. The compound of any one of embodiments 1-6, or a pharmaceutically acceptable salt, hydrate, solvate, or prodrug thereof, wherein L₂ is

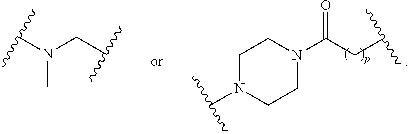

10. The compound of any one of embodiments 1-9, or a pharmaceutically acceptable salt, hydrate, solvate, or prodrug thereof, wherein R₁ is

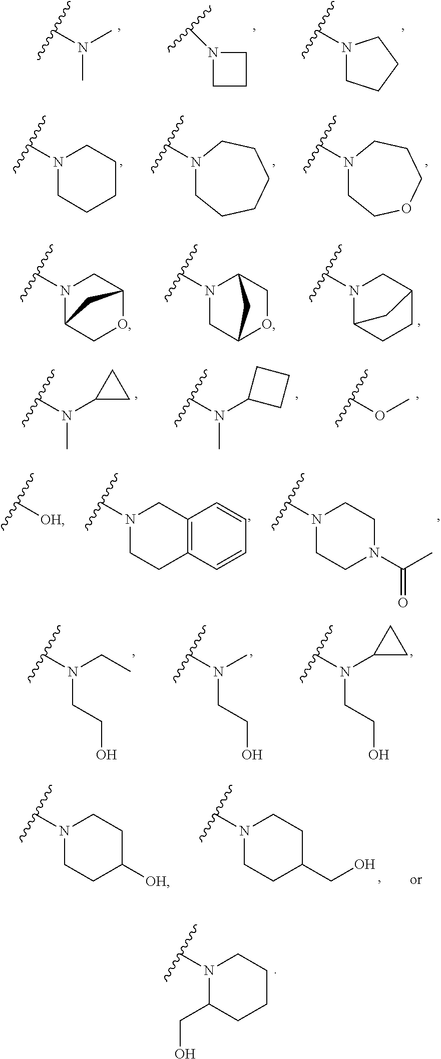

11. The compound of embodiment 10, or a pharmaceutically acceptable salt, hydrate, solvate, or prodrug thereof, wherein R₁ is

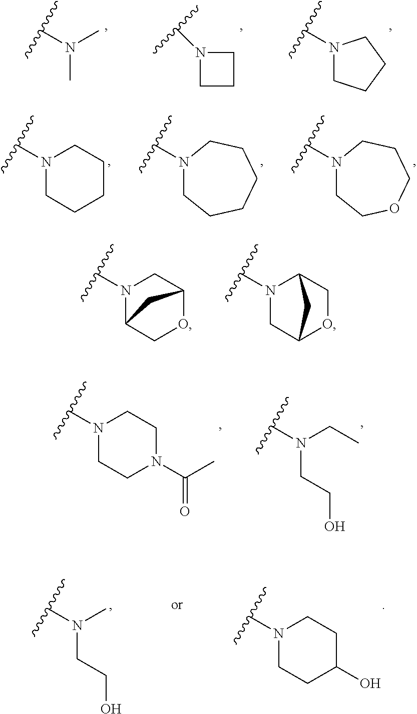

12. The compound of embodiment 1, 10, or 11, or a pharmaceutically acceptable salt, hydrate, solvate, or prodrug thereof, wherein L₁ is

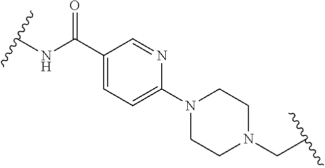 [SMILES: *CN1CCN(c2ccc(C(=O)N*)cn2)CC1]

and L₂ is a bond or

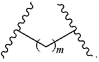

13. The compound of embodiment 1, 10, or 11, or a pharmaceutically acceptable salt, hydrate, solvate, or prodrug thereof, wherein L₁ is

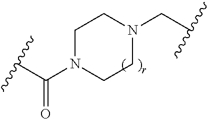

### and L₂ is

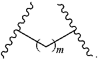

14. The compound of embodiment 1, 10, or 11, or a pharmaceutically acceptable salt, hydrate, solvate, or prodrug thereof, wherein L₁ is

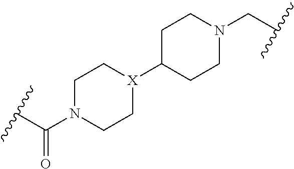

and L₂ is

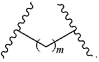

15. The compound of embodiment 1, 10, or 11, or a pharmaceutically acceptable salt, hydrate, solvate, or prodrug thereof, wherein L₁ is

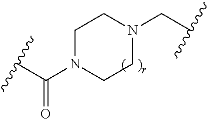

### and L₂ is

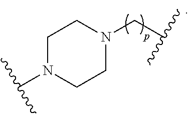

16. The compound of embodiment 1, 10, or 11, or a pharmaceutically acceptable salt, hydrate, solvate, or prodrug thereof, wherein:
L₂ is

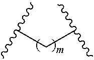

(e.g., wherein m is 1), and L₁ is

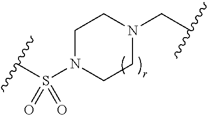

(e.g., wherein r is 1);
L₂ is

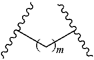

(e.g., wherein m is 3), and L₁ is

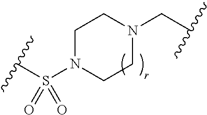

(e.g., wherein r is 1);
L₂ is

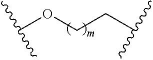

(e.g., wherein m is 2), and L₁ is

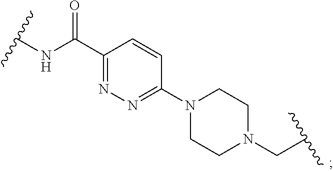 [SMILES: *CN1CCN(c2ccc(C(=O)N*)nn2)CC1]

L₂ is

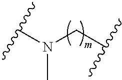

(e.g., wherein m is 3), and L₁ is

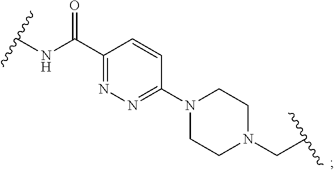 [SMILES: *CN1CCN(c2ccc(C(=O)N*)nn2)CC1]

L₂ is

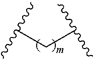

(e.g., wherein m is 4), and L₁ is

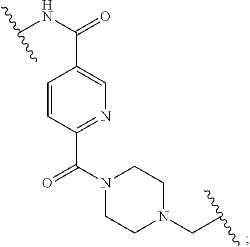 [SMILES: *CN1CCN(C(=O)c2ccc(C(=O)N*)cn2)CC1]

L₂ is

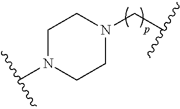

(e.g., wherein p is 2), and L₁ is

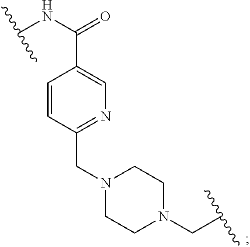 [SMILES: *CN1CCN(Cc2ccc(C(=O)N*)cn2)CC1]

L₂ is

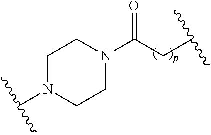

(e.g., wherein p is 1), and L₁ is

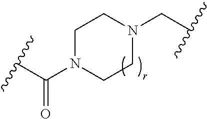

(e.g., wherein r is 1);
L₂ is

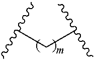

(e.g., wherein m is 3), and L₁ is

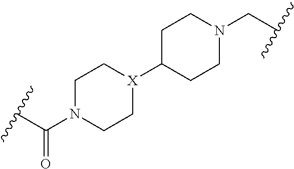

(e.g., wherein X is CH);
L₂ is

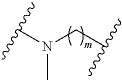

(e.g., wherein m is 2), and L₁ is

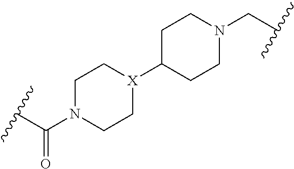

(e.g., wherein X is CH);
L₂ is

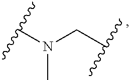 [SMILES: *CN(*)C]

### and L₁ is

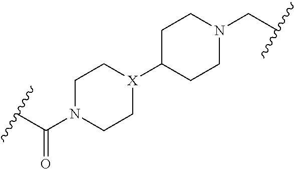

(e.g., wherein X is CH);
L₂ is

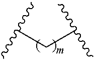

(e.g., wherein m is 3), and L₁ is

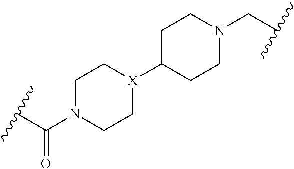

(e.g., wherein X is N); or
L₂ is

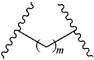

(e.g., wherein m is 3), and L₁ is

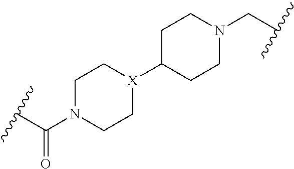

(e.g., wherein X is N).

17. The compound of any one of embodiments 1-16, or a pharmaceutically acceptable salt, hydrate, solvate, or prodrug thereof, wherein R₂ is NO₂.

18. The compound of any one of embodiments 1-16, or a pharmaceutically acceptable salt, hydrate, solvate, or prodrug thereof, wherein R₂ is SO₂CF₃.

19. The compound of any one of embodiments 1-18, or a pharmaceutically acceptable salt, hydrate, solvate, or prodrug thereof, wherein R₃ is F, Cl, or CF₃.

20. The compound of embodiment 19, or a pharmaceutically acceptable salt, hydrate, solvate, or prodrug thereof, wherein R₃ is Cl.

21. The compound of any one of embodiments 1-20, or a pharmaceutically acceptable salt, hydrate, solvate, or prodrug thereof, wherein R₄ is CH₃.

22. The compound of any one of embodiments 1-20, or a pharmaceutically acceptable salt, hydrate, solvate, or prodrug thereof, wherein R₄ is H.

23. The compound of any one of embodiments 1-22, or a pharmaceutically acceptable salt, hydrate, solvate, or prodrug thereof, wherein R₅ is H.

24. The compound of any one of embodiments 1-22, or a pharmaceutically acceptable salt, hydrate, solvate, or prodrug thereof, wherein R₅ is optionally substituted C₁₋₆ alkyl.

25. The compound of embodiment 24, or a pharmaceutically acceptable salt, hydrate, solvate, or prodrug thereof, wherein R₅ is CH₃.

26. The compound of any one of embodiments 1-25, or a pharmaceutically acceptable salt, hydrate, solvate, or prodrug thereof, wherein X is N.

27. The compound of any one of embodiments 1-25, or a pharmaceutically acceptable salt, hydrate, solvate, or prodrug thereof, wherein X is CH.

28. The compound of any one of embodiments 1-27, or a pharmaceutically acceptable salt, hydrate, solvate, or prodrug thereof, wherein r is 1.

29. The compound of any one of embodiments 1-27, or a pharmaceutically acceptable salt, hydrate, solvate, or prodrug thereof, wherein r is 2.

30. The compound of embodiment 1, or a pharmaceutically acceptable salt, hydrate, solvate, or prodrug thereof, wherein the compound is:

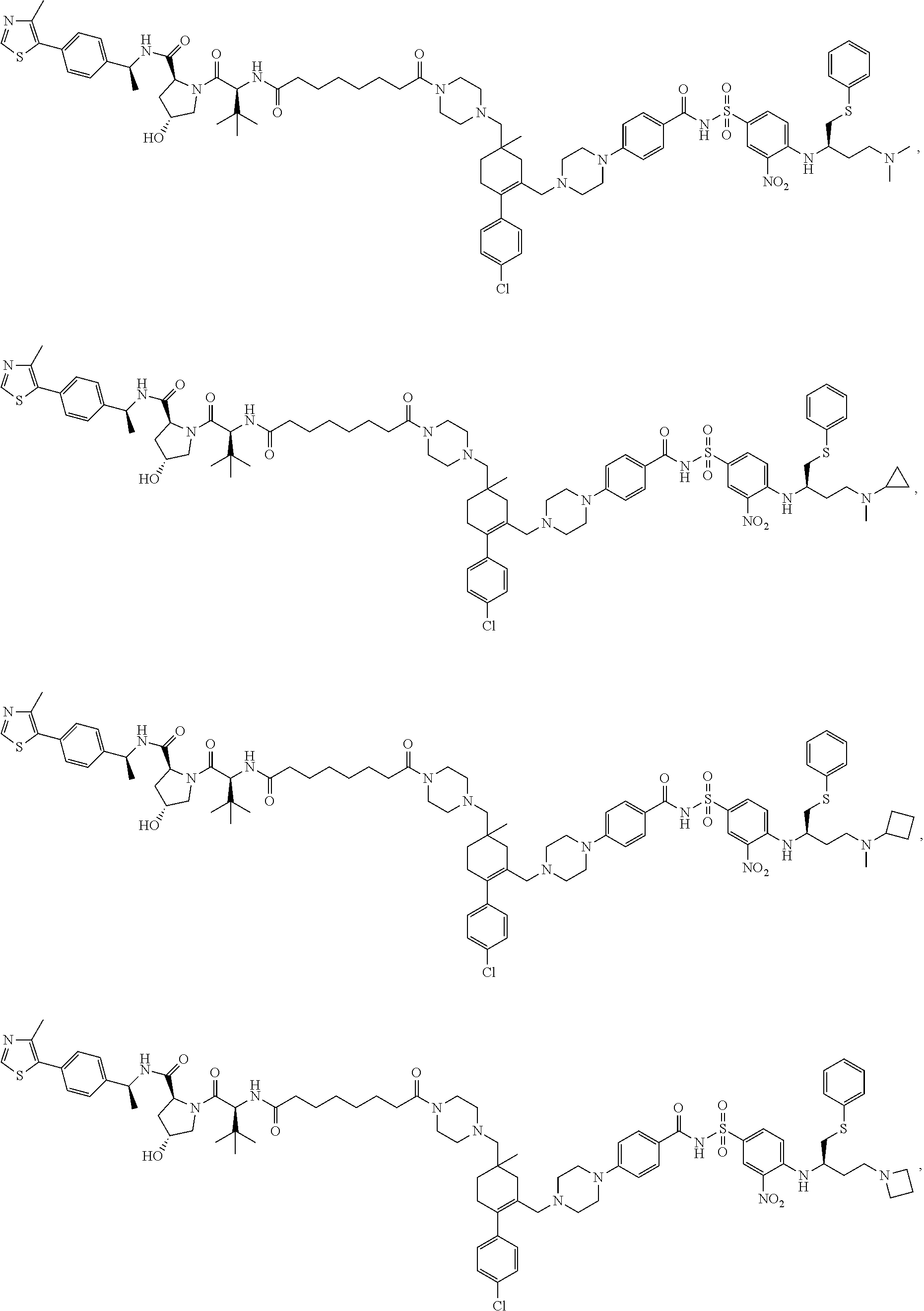

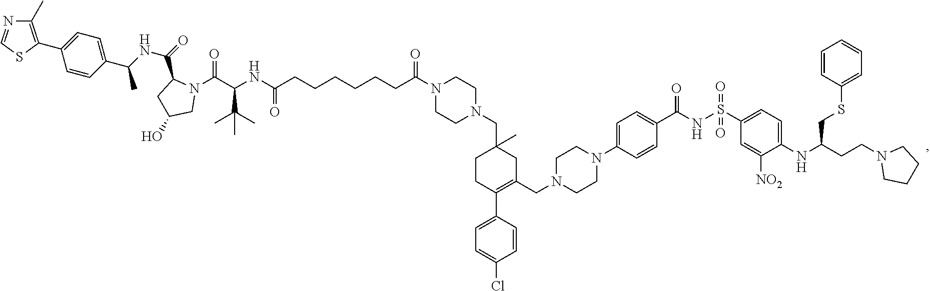 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCCC7)CSc7ccccc7)c([N+](=O)[O-])c6)cc5)CC4)C3)CC2)C(C)(C)C)cc1]

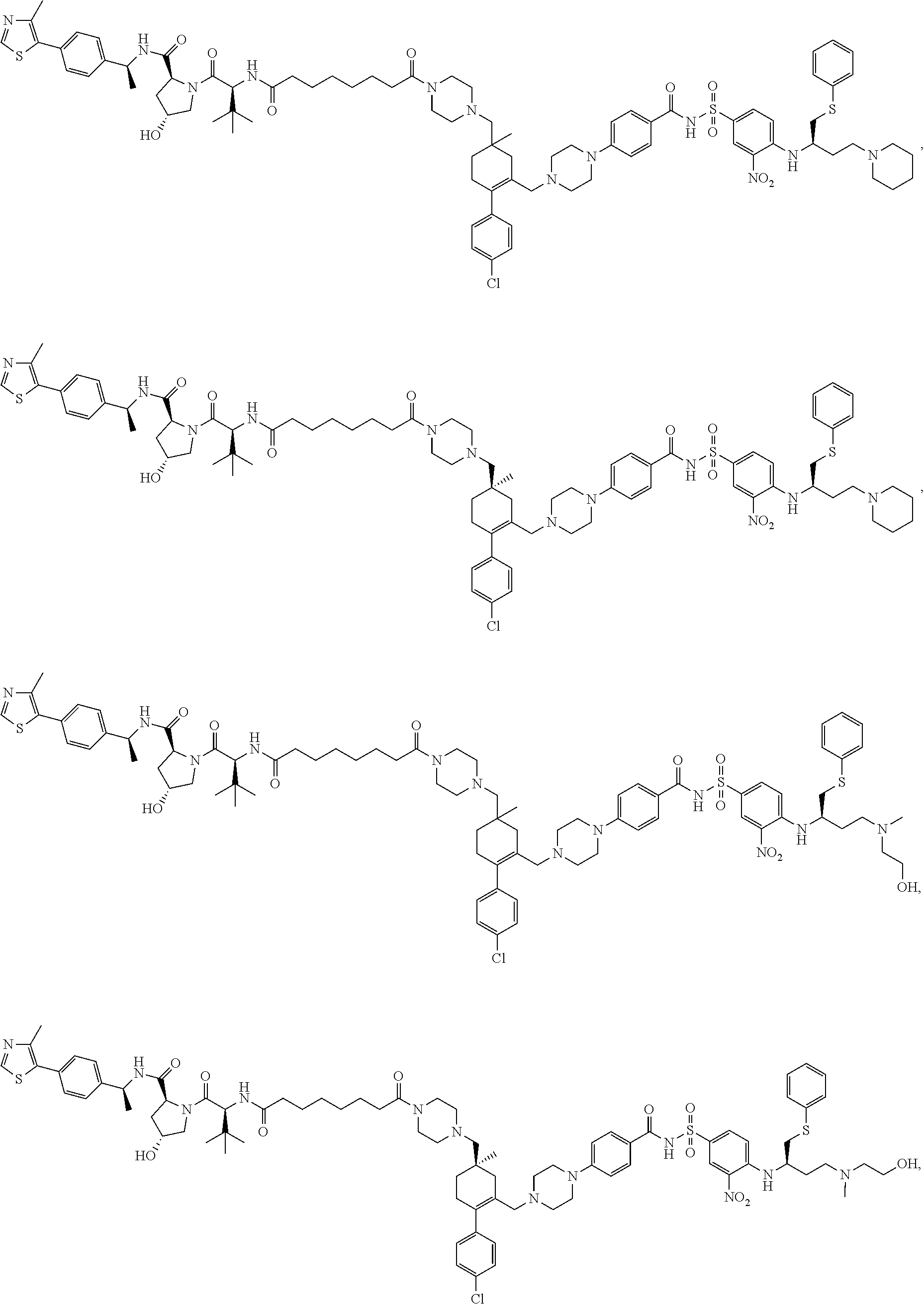

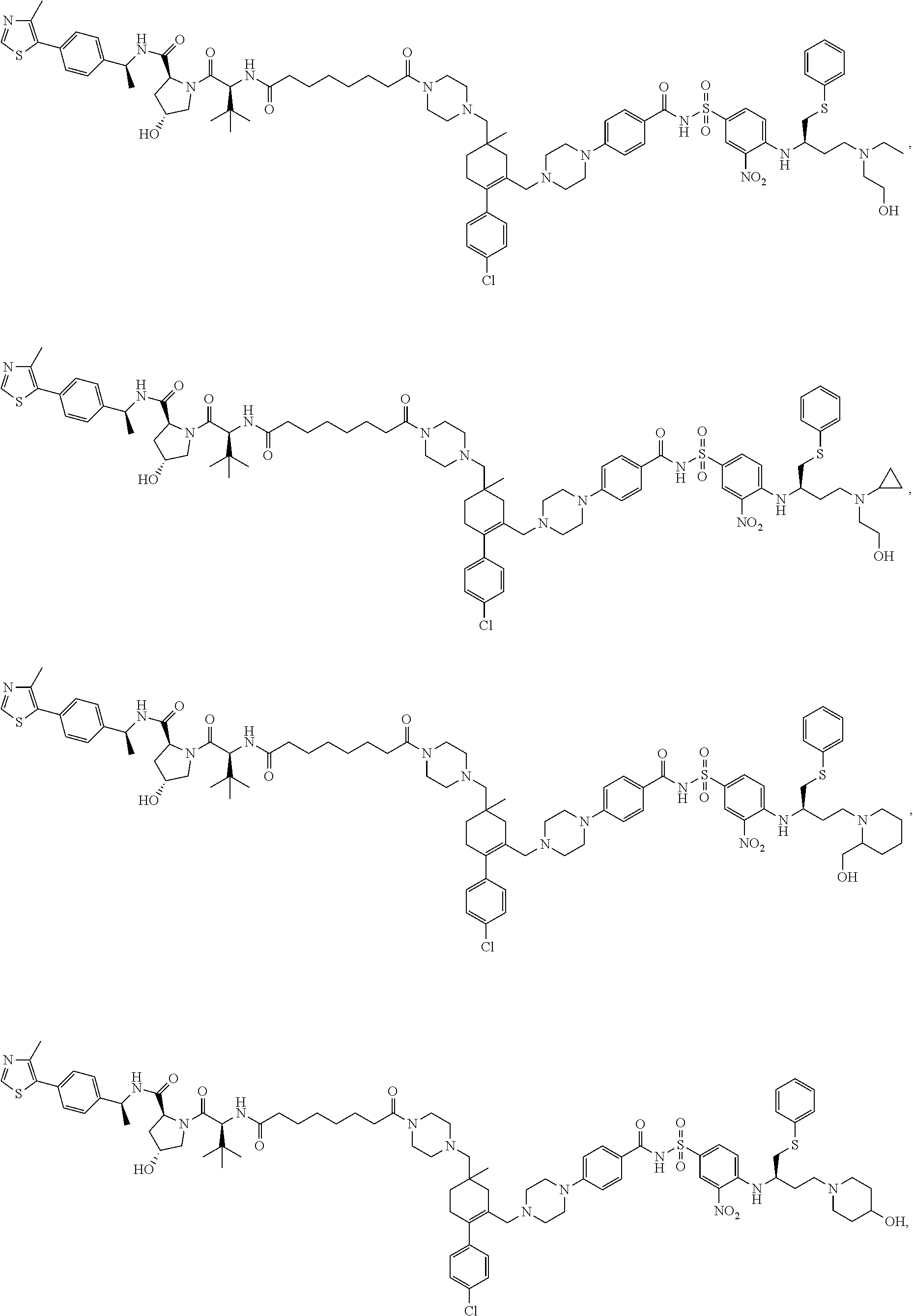

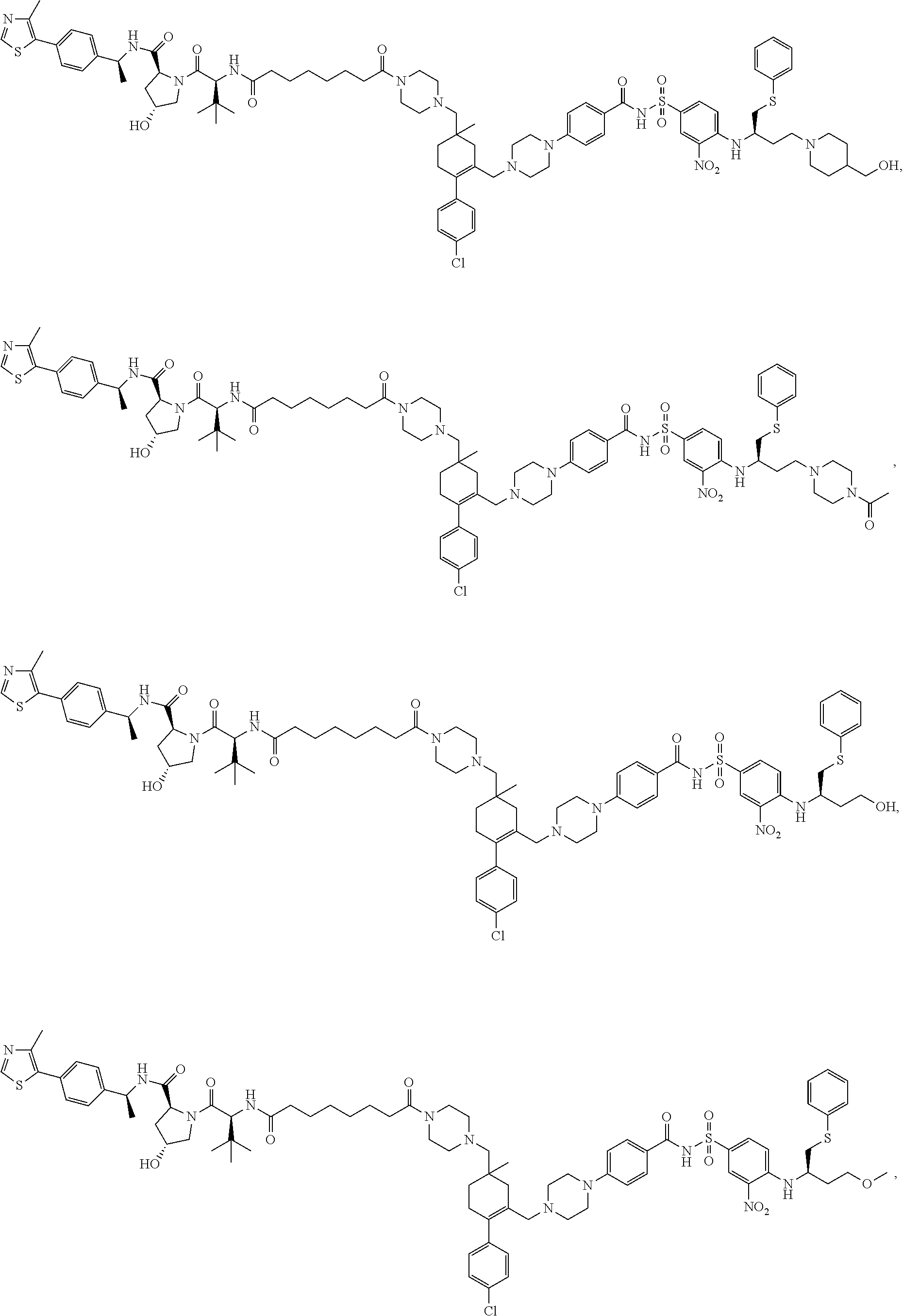

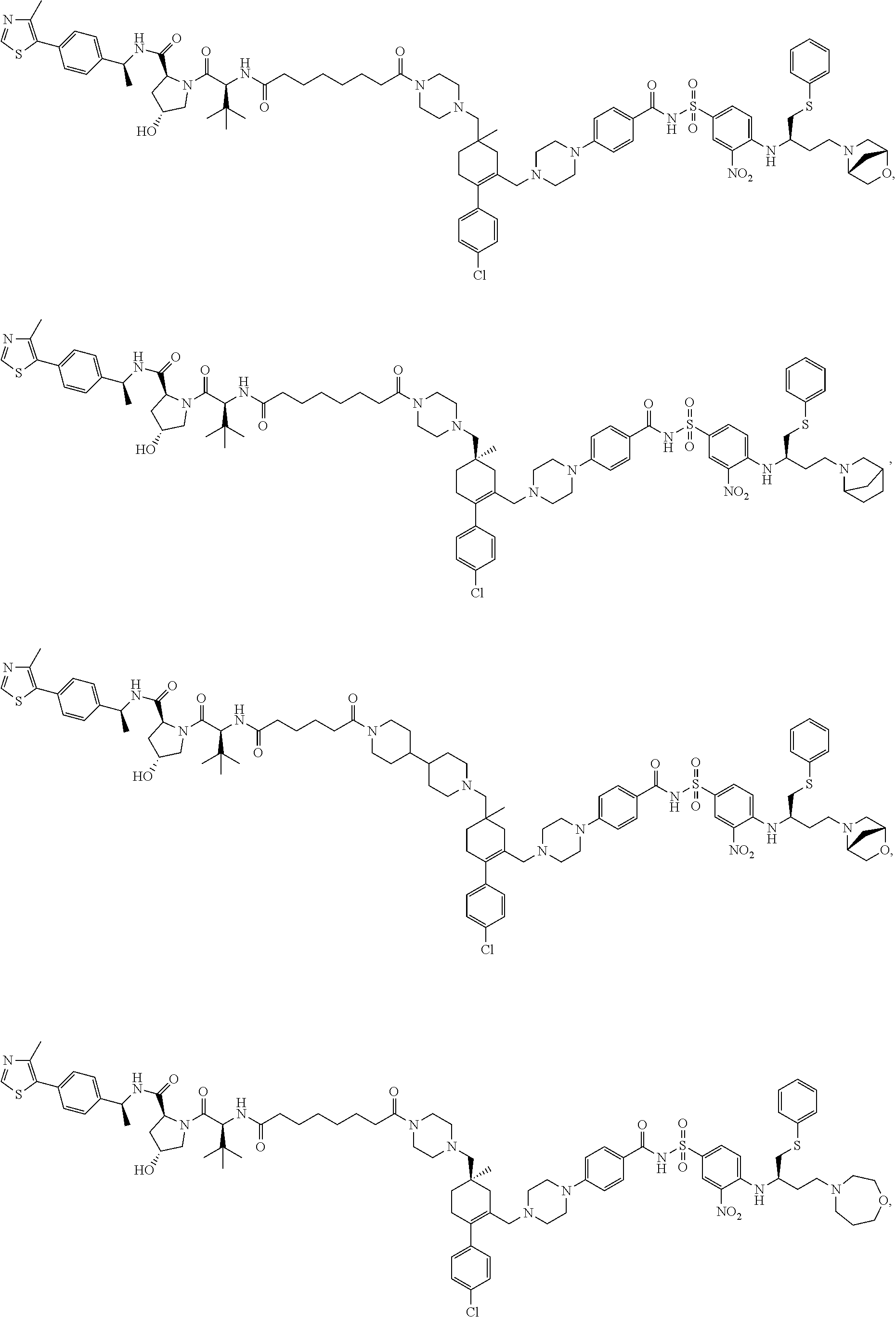

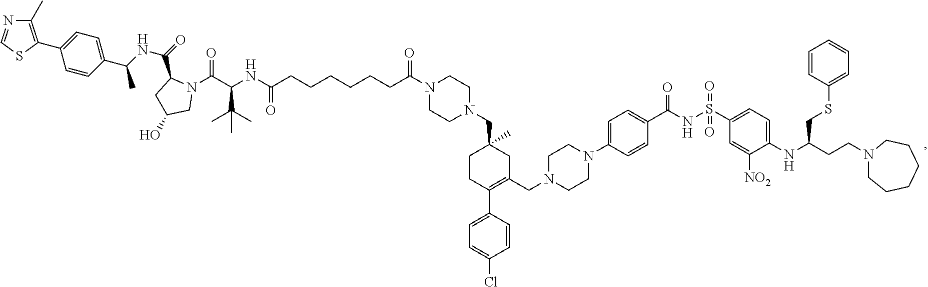 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCCC7)CSc7ccccc7)c([N+](=O)[O-])c6)cc5)CC4)C3)CC2)C(C)(C)C)cc1]

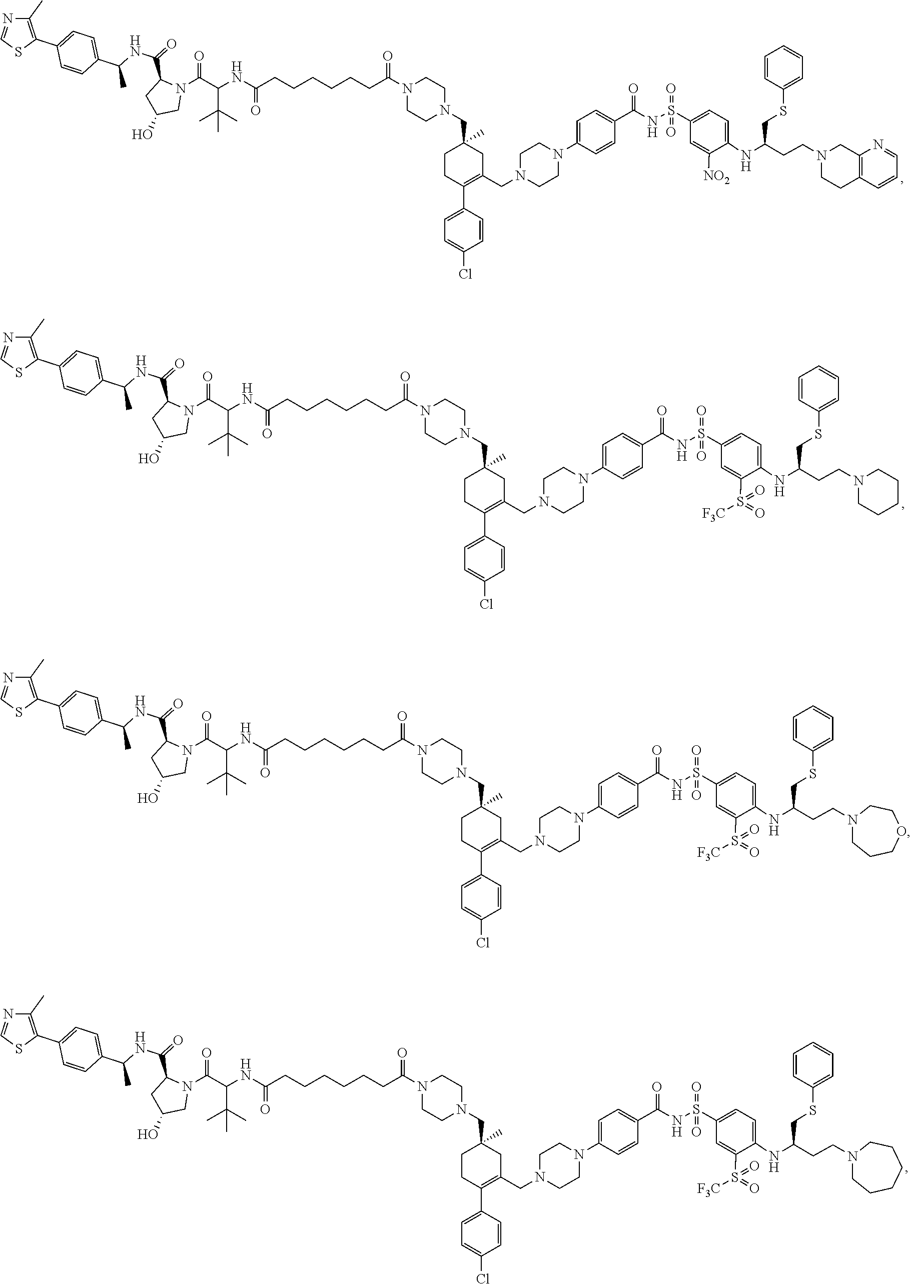

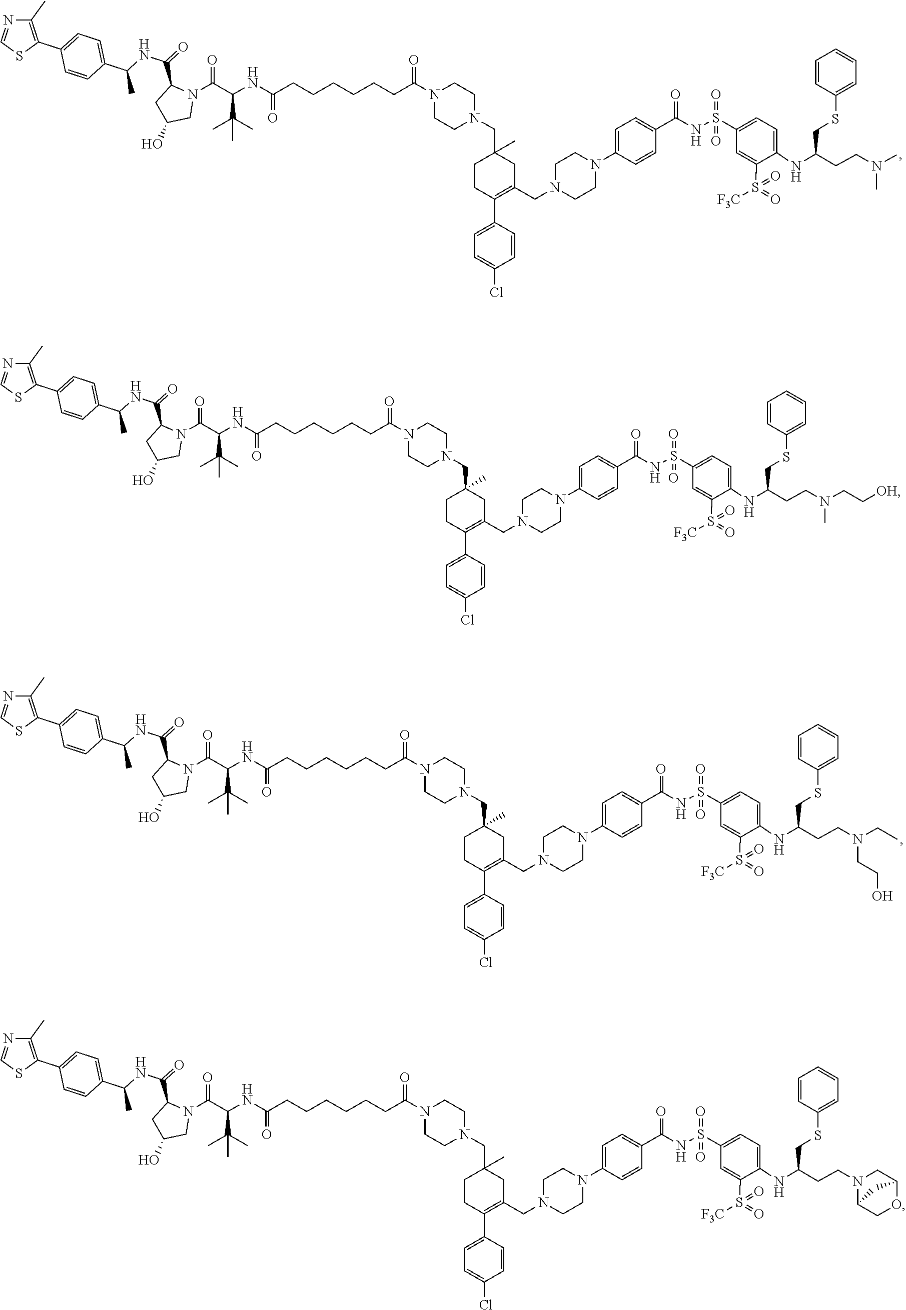

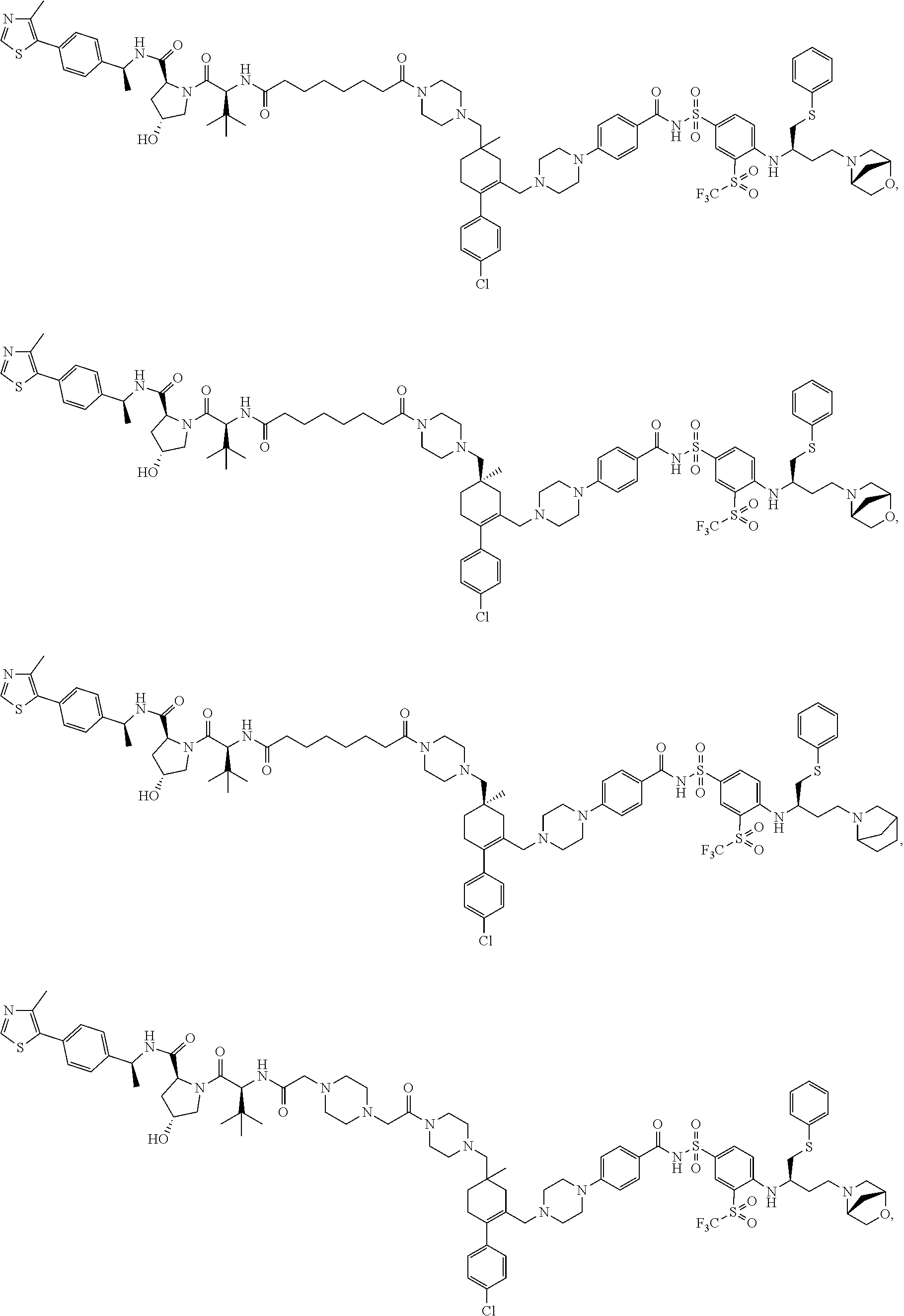

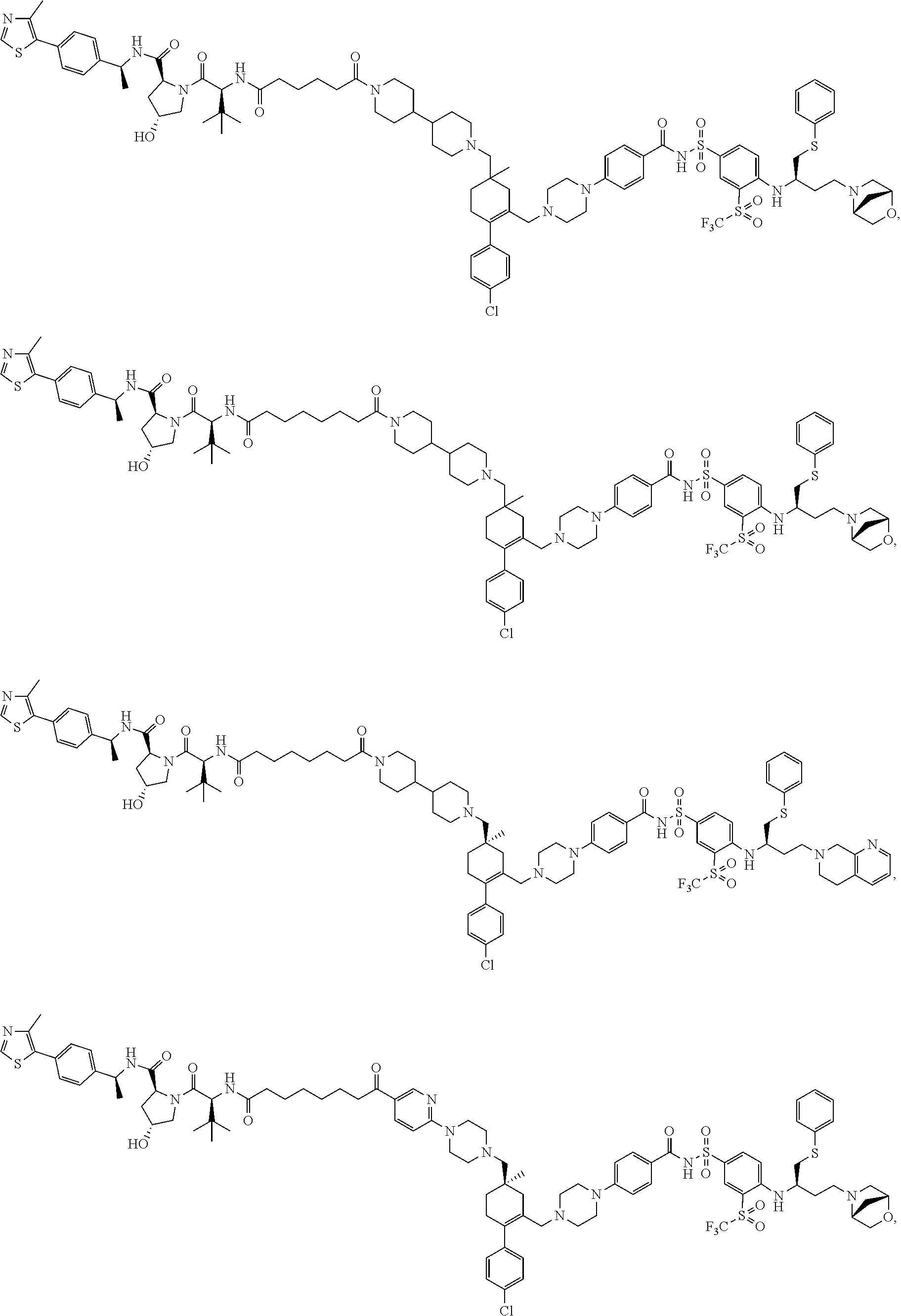

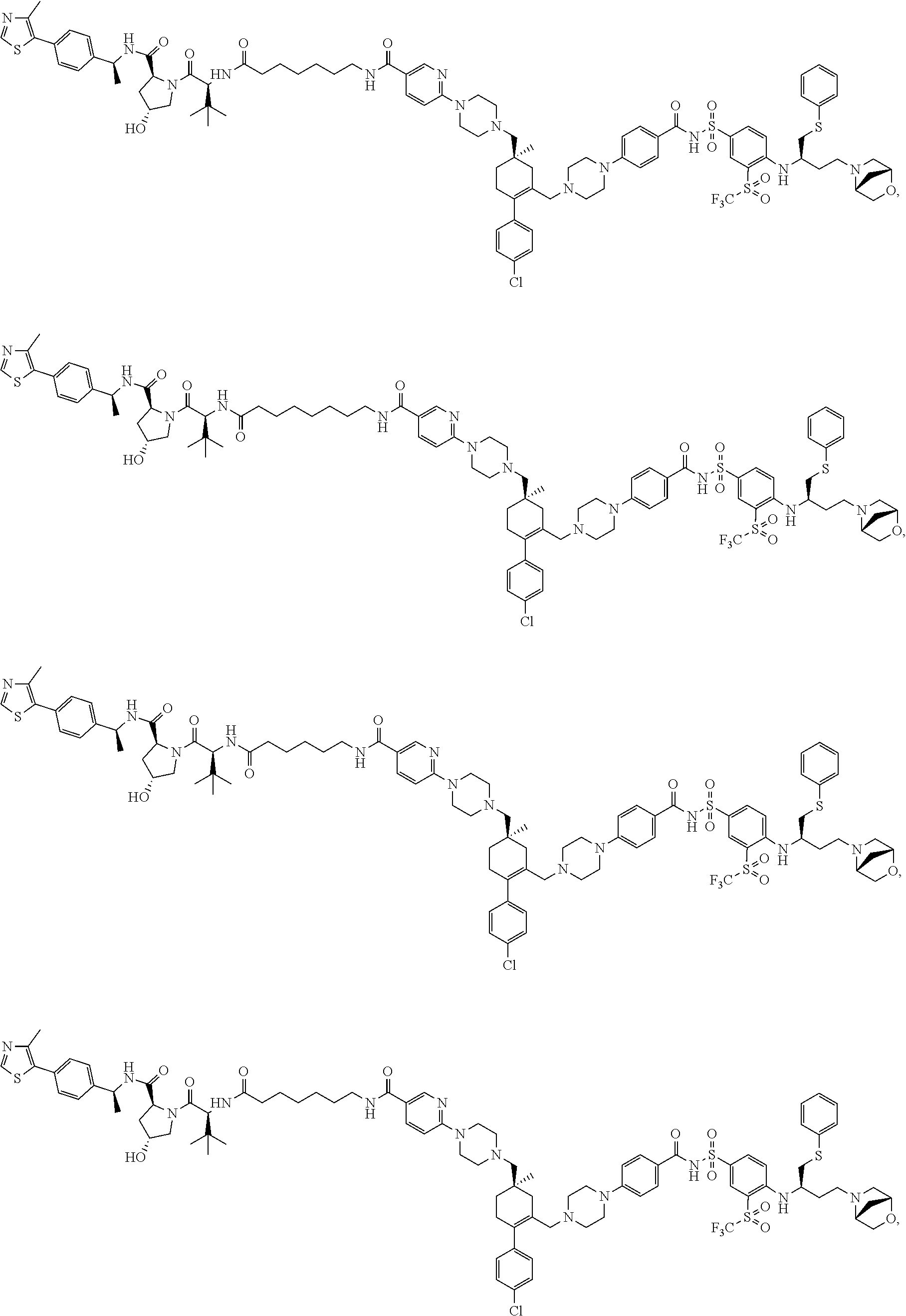

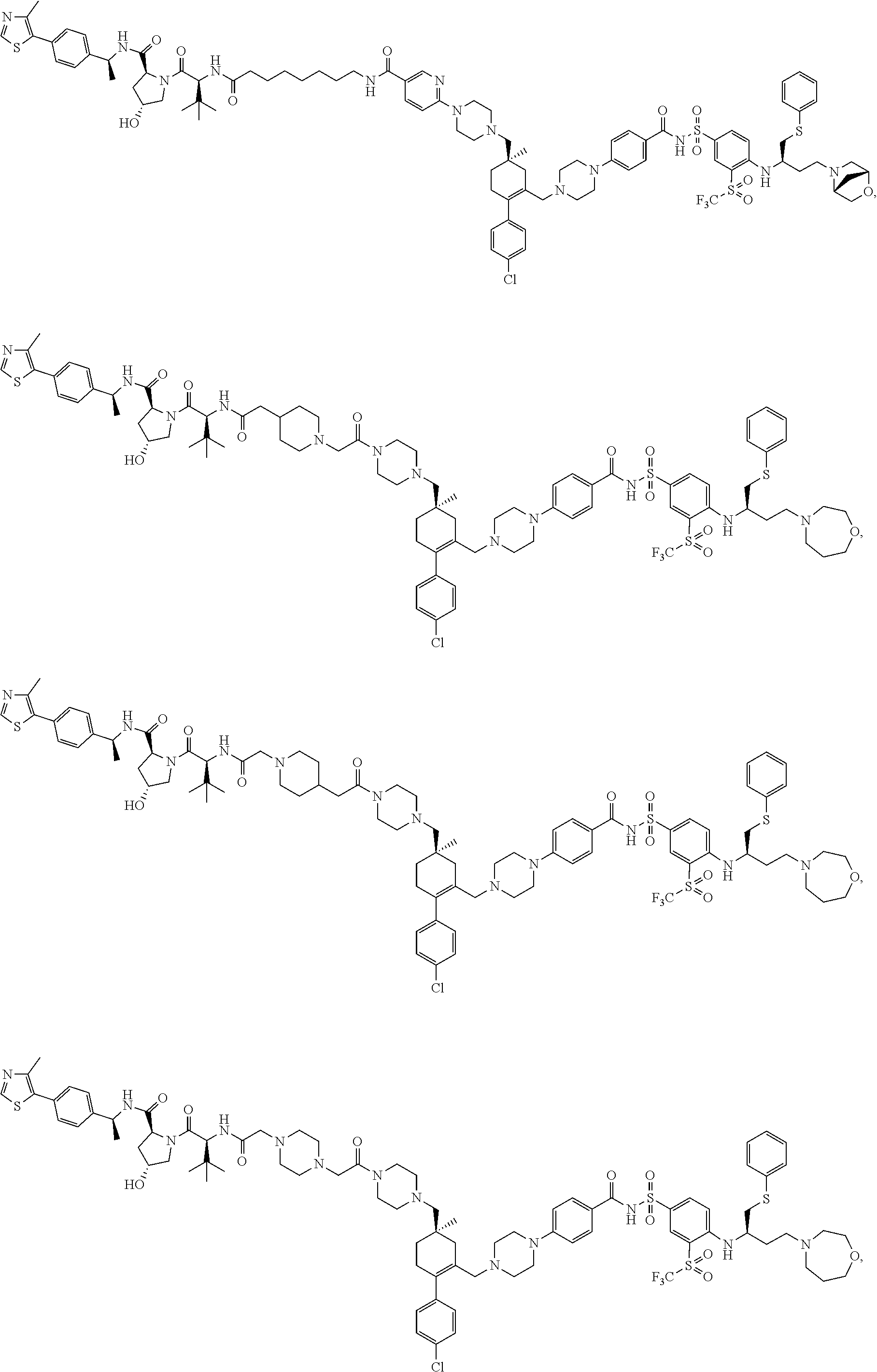

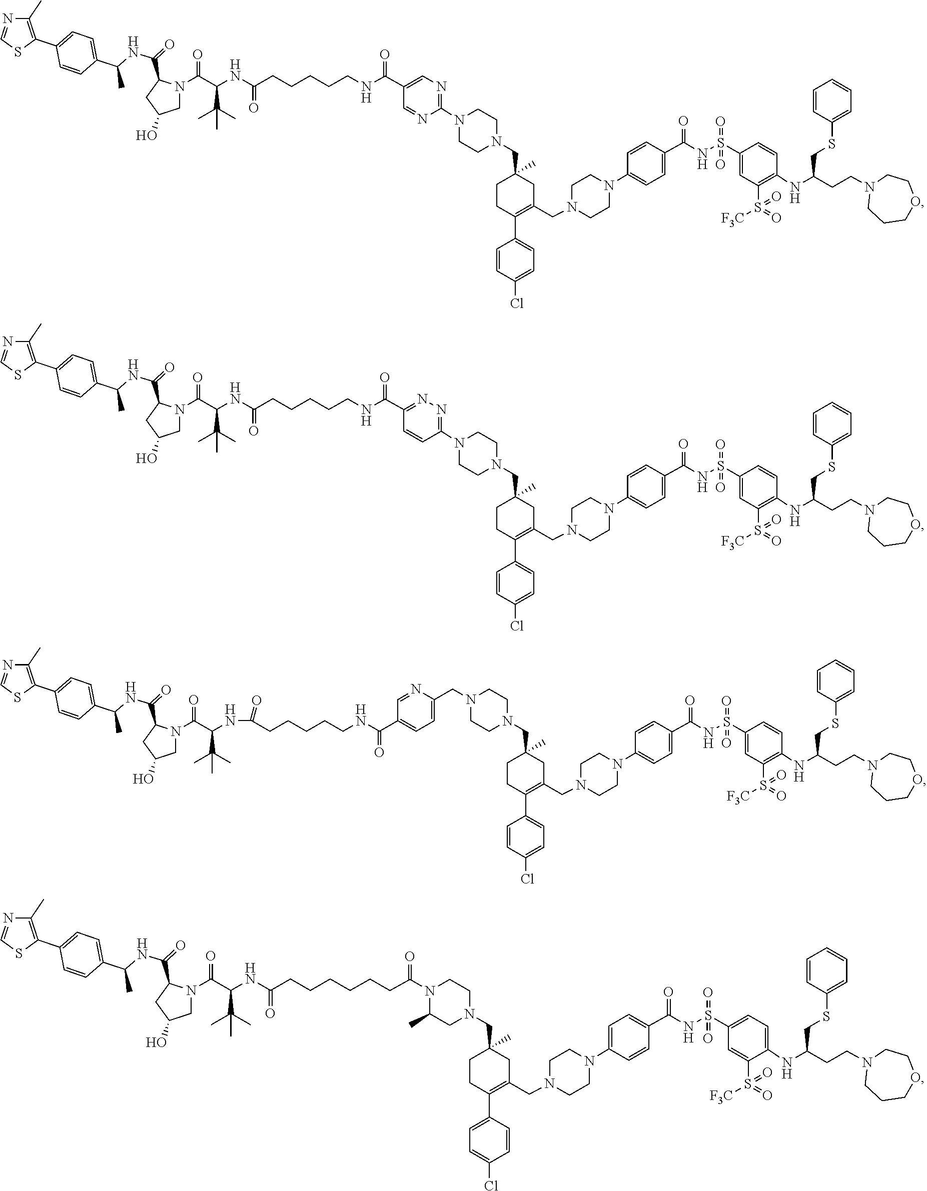

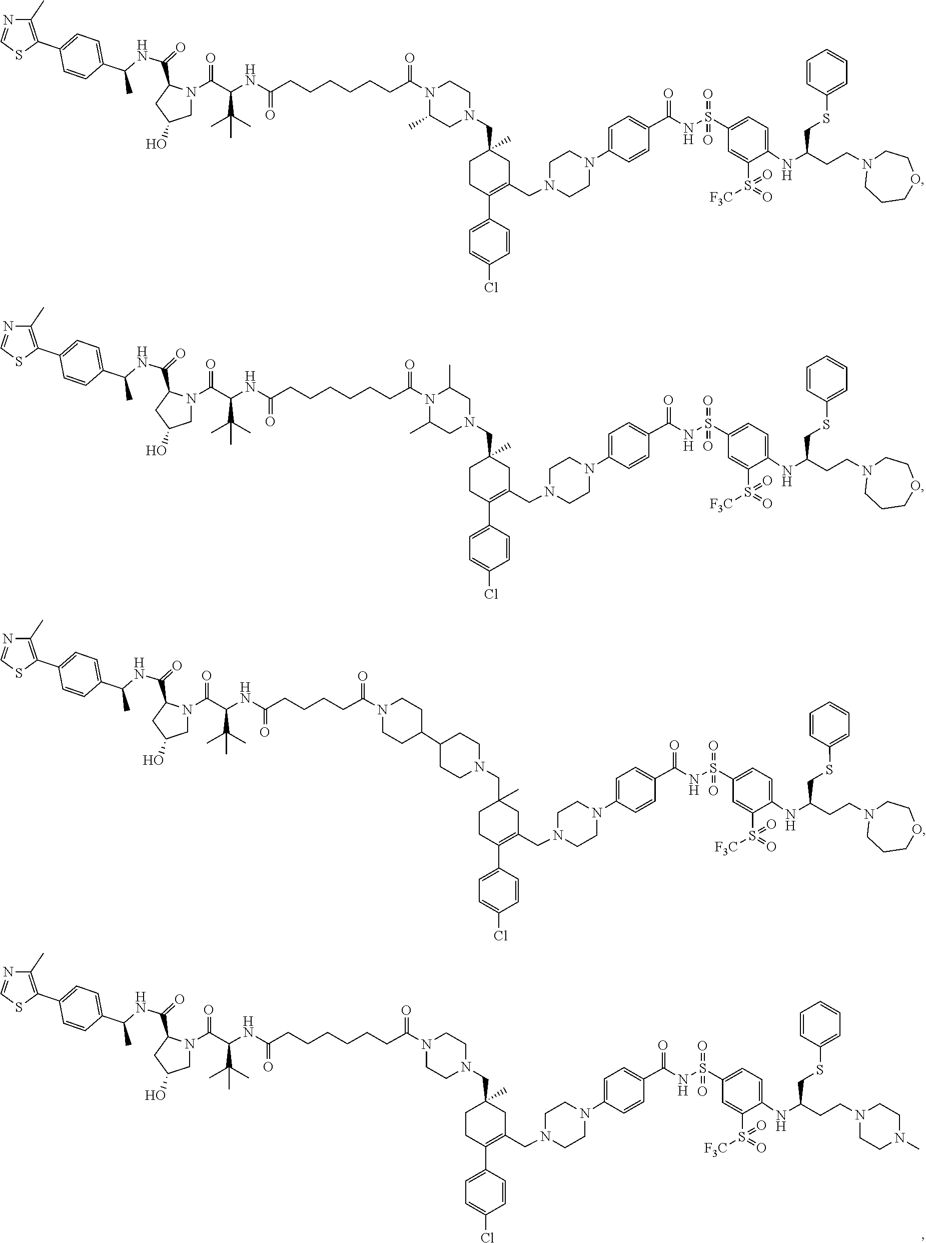

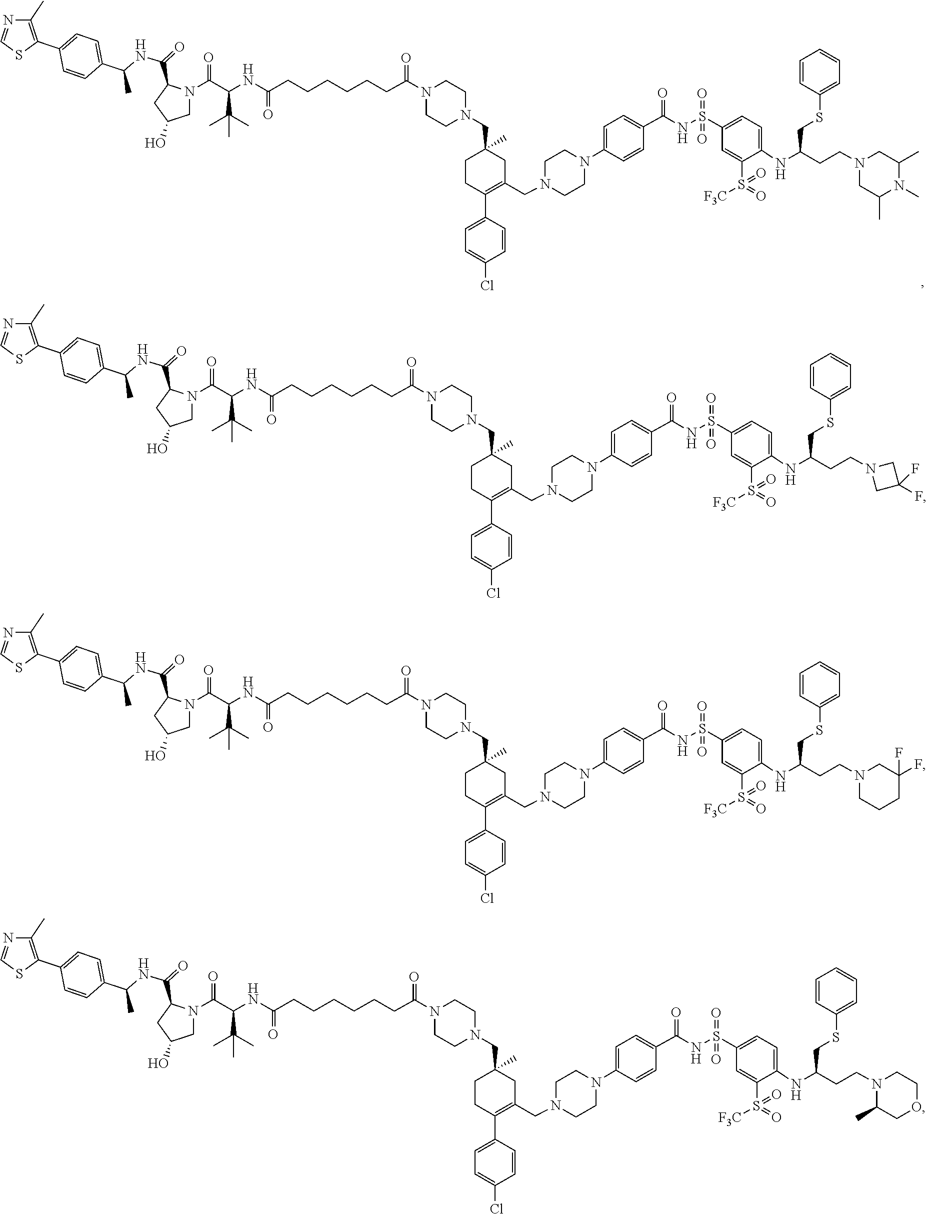

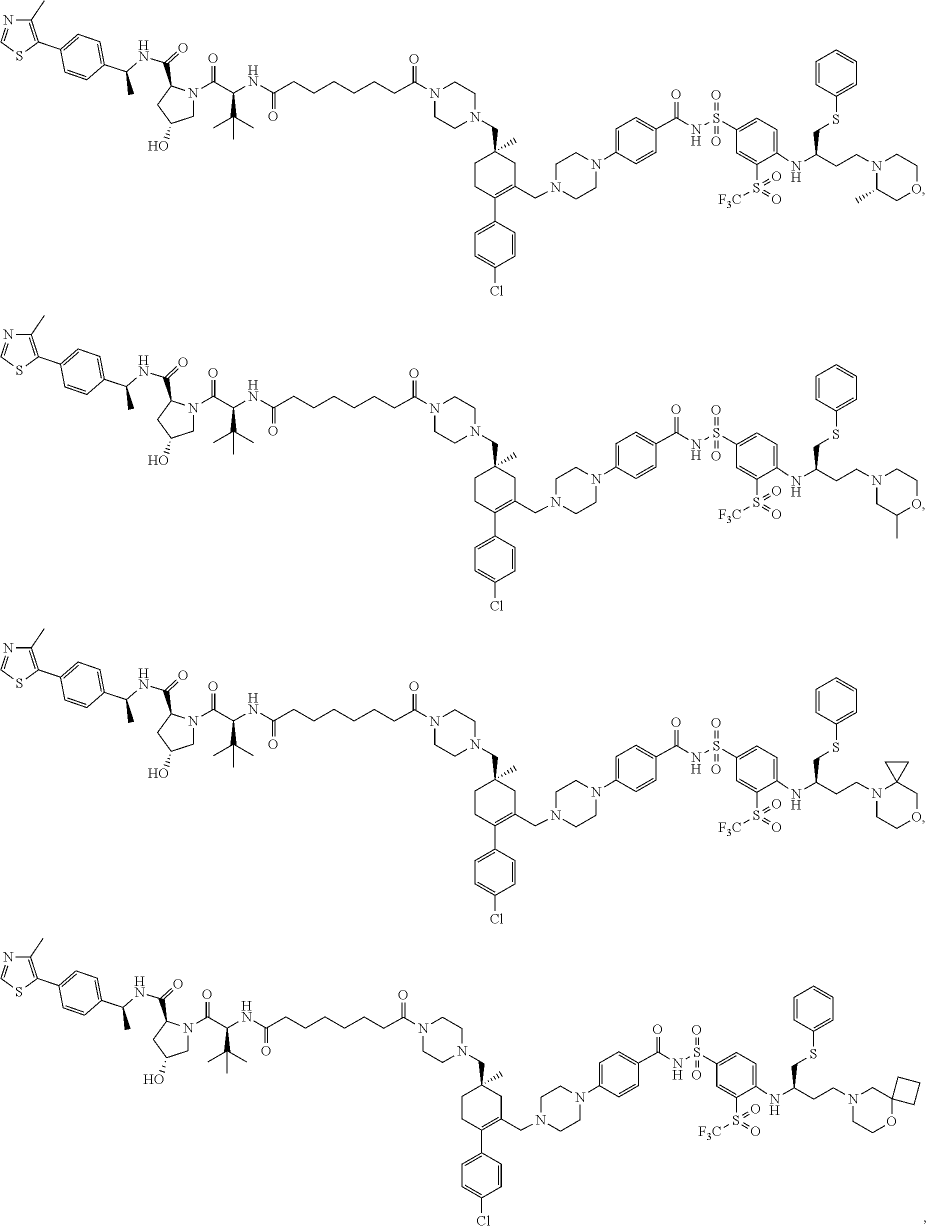

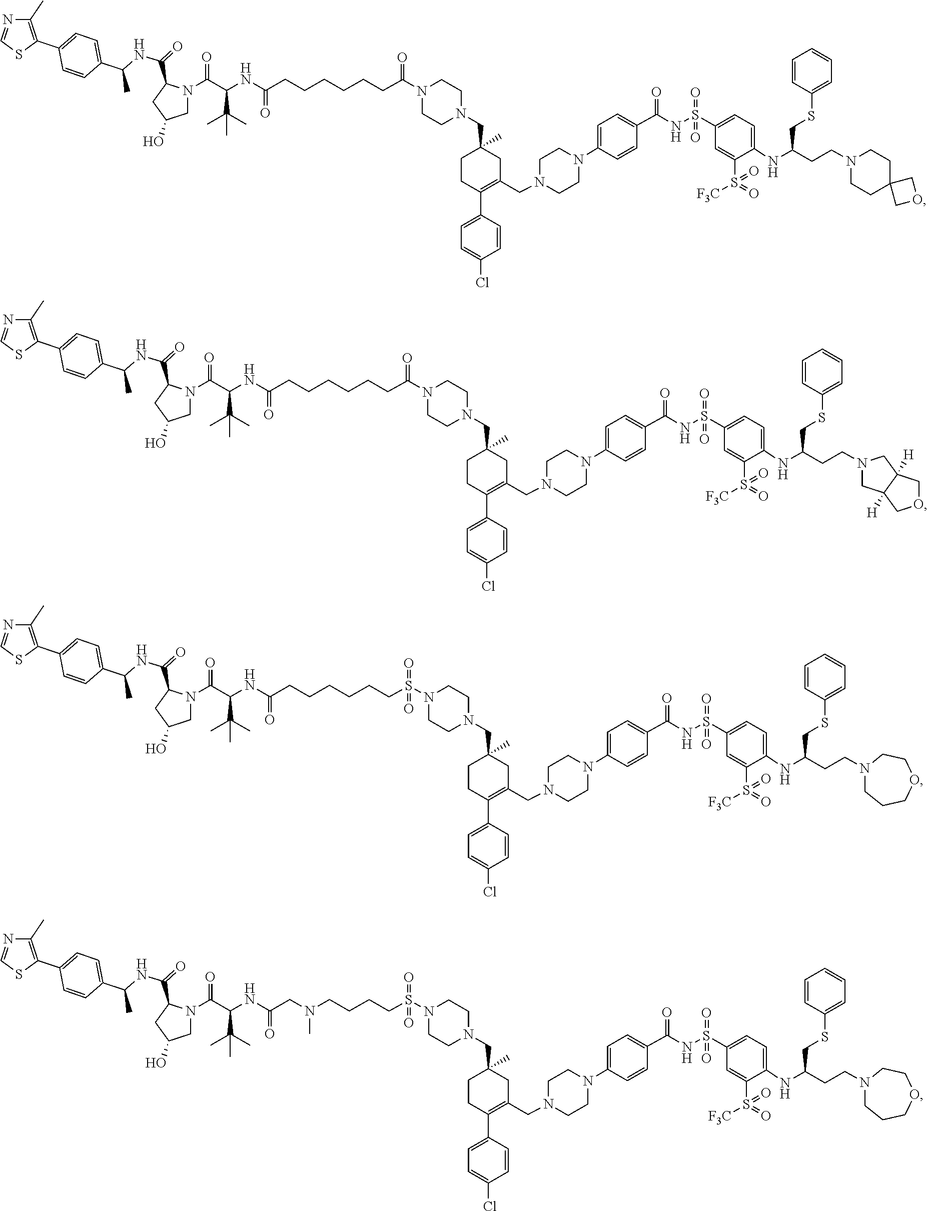

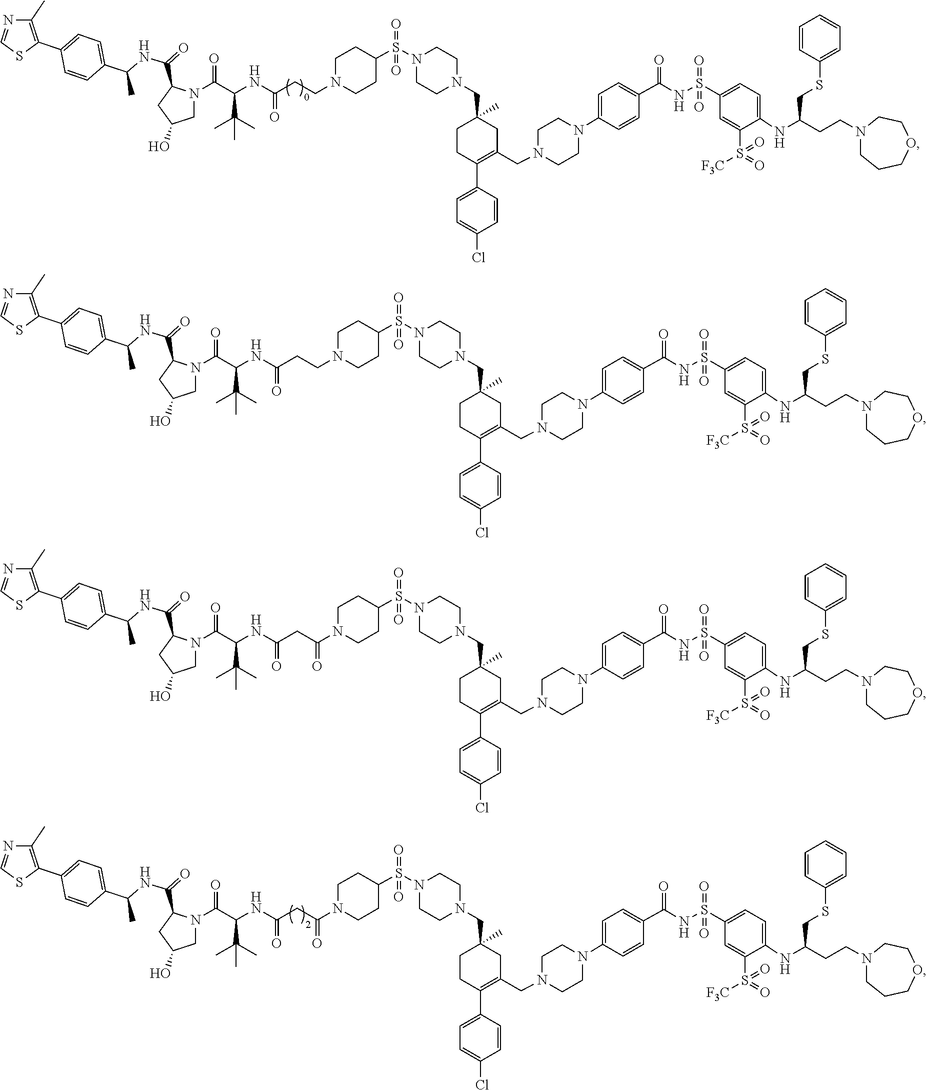

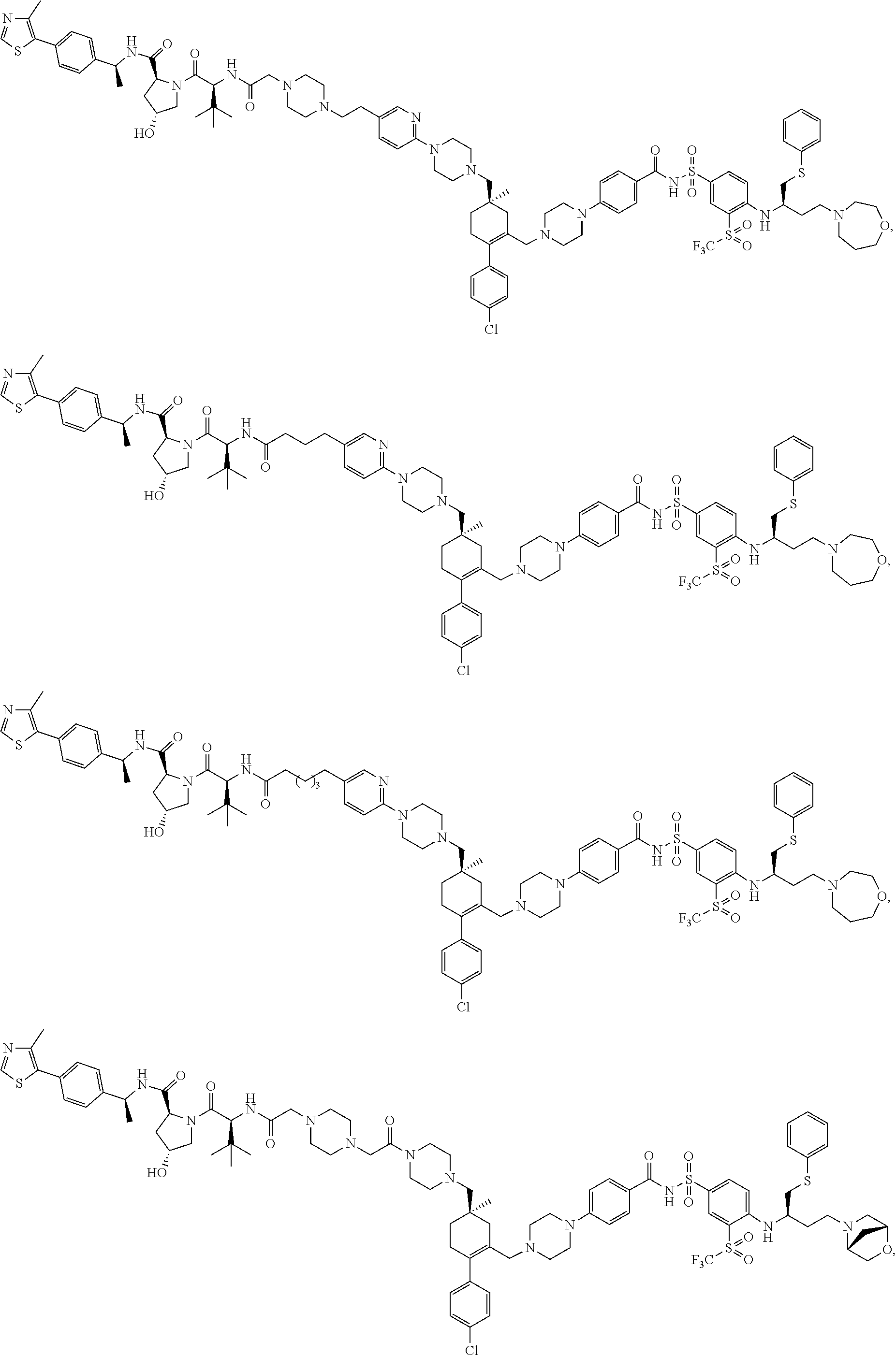

or a pharmaceutically acceptable salt, hydrate, solvate, or prodrug thereof.

31. The compound of embodiment 1, or a pharmaceutically acceptable salt, hydrate, solvate, or prodrug thereof, wherein the compound is:

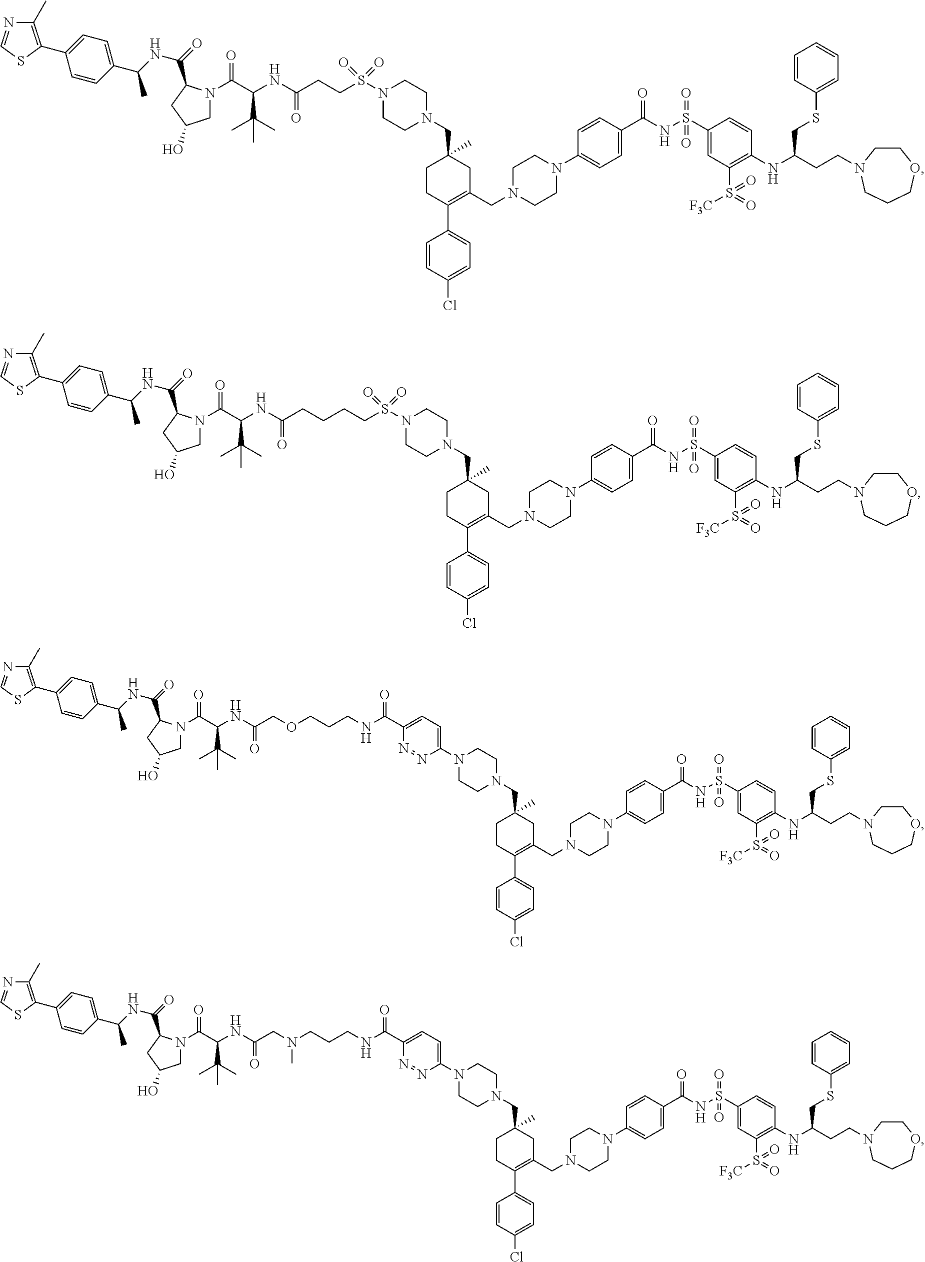

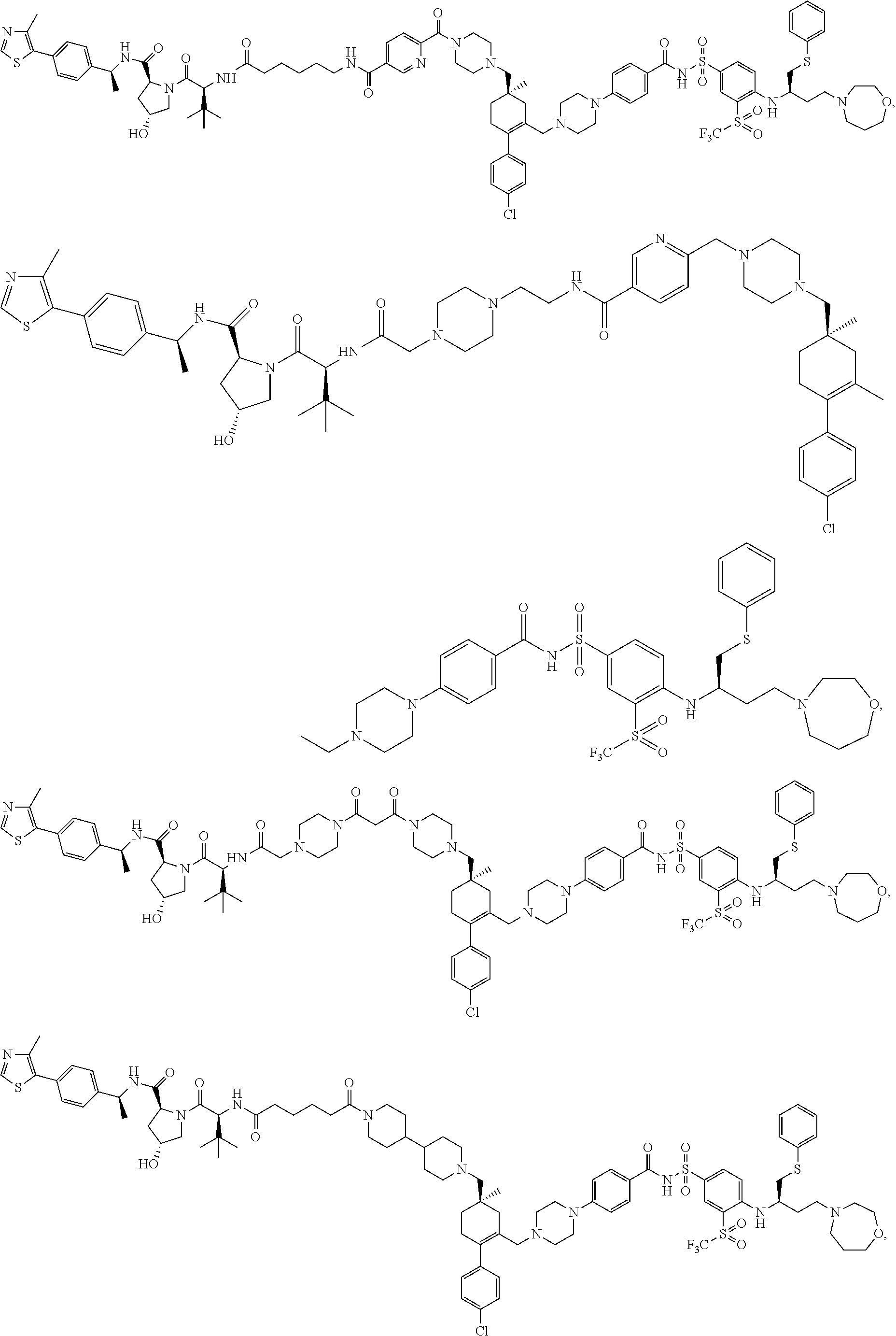

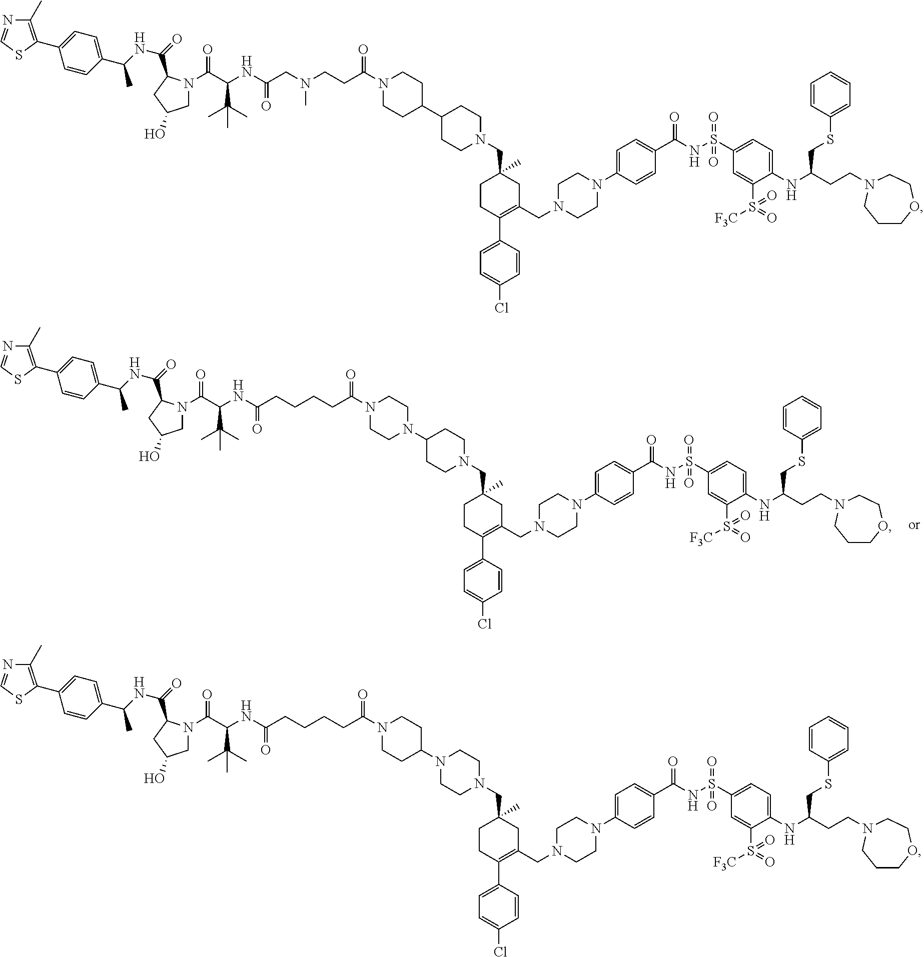

or a pharmaceutically acceptable salt, hydrate, solvate, or prodrug thereof.

32. A compound of Formula (II), or a pharmaceutically acceptable salt, hydrate, solvate, or prodrug thereof:

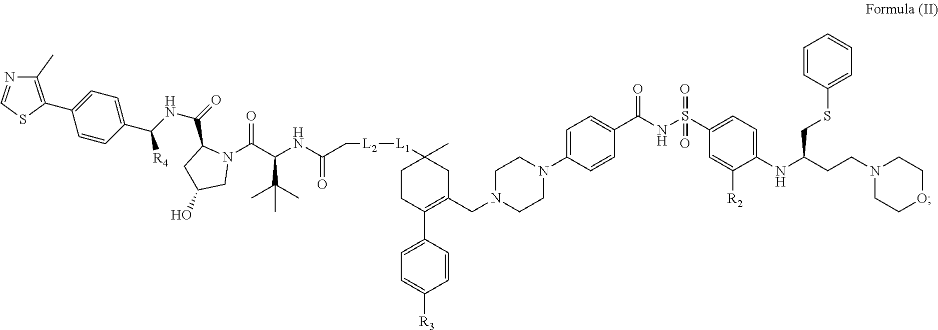

wherein R₂ is NO₂, SO₂CF₃, or SO₂CF₂Cl;
R₃ is Cl, F, CF₂H, CFH₂, or CF₃;
R₄ is H or CH₃;
L₁ is

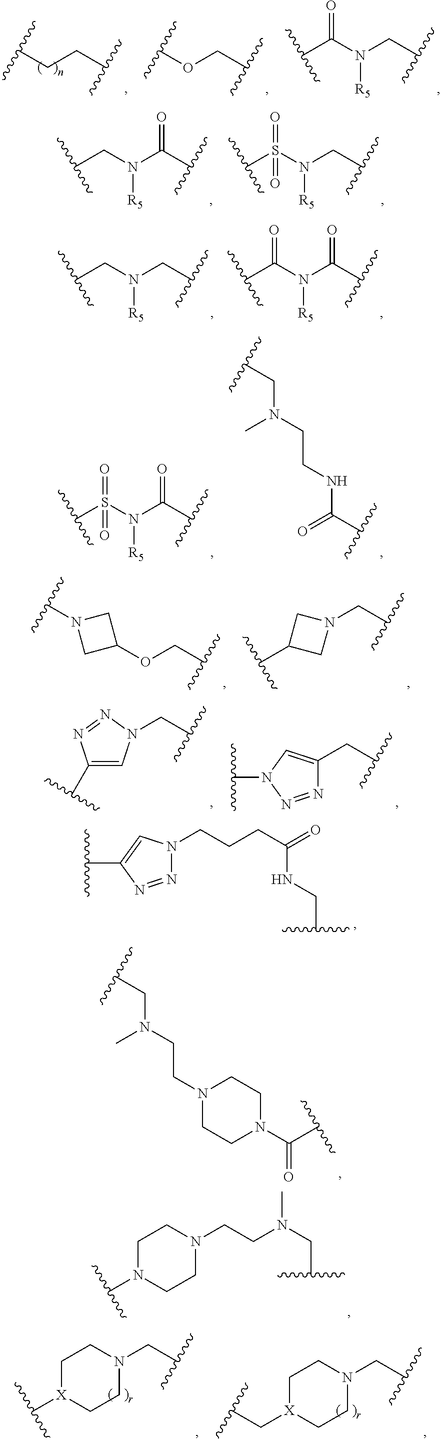

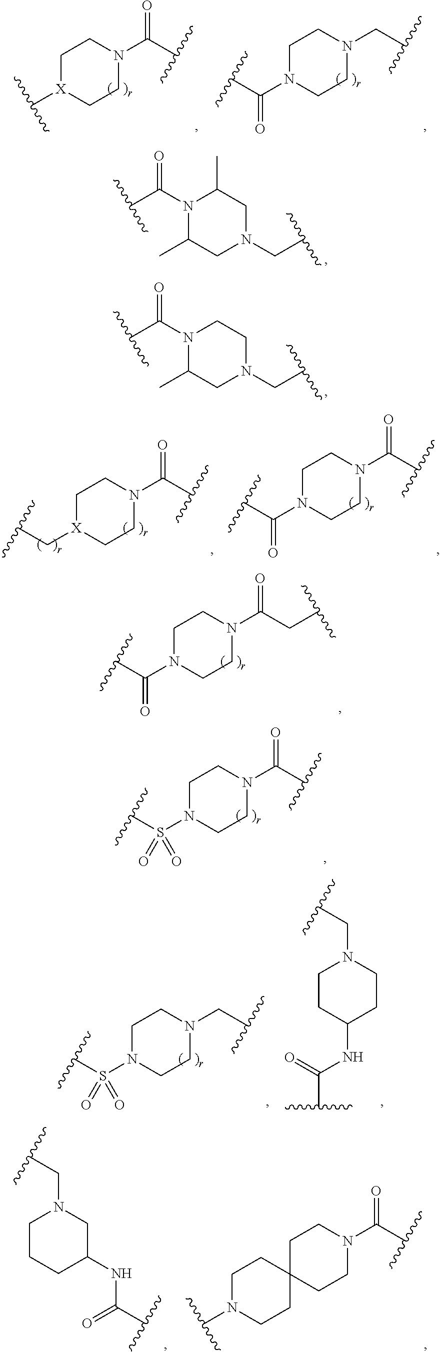

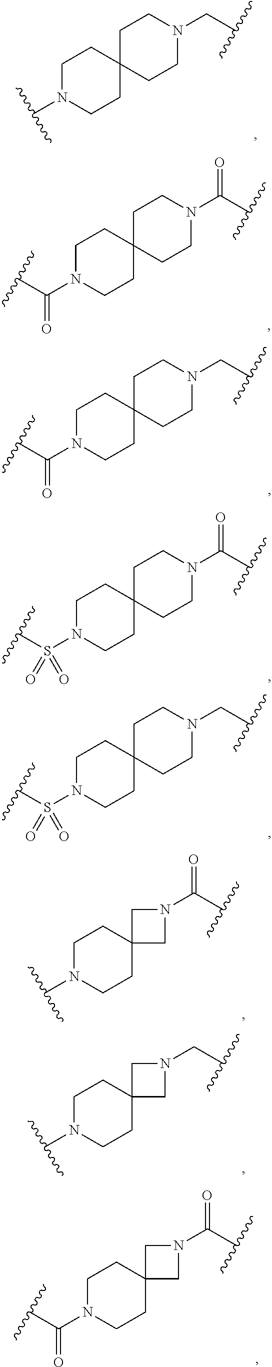

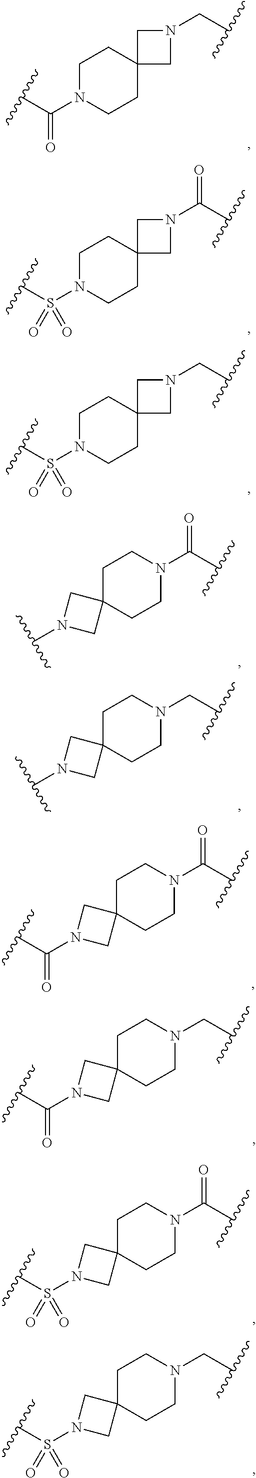

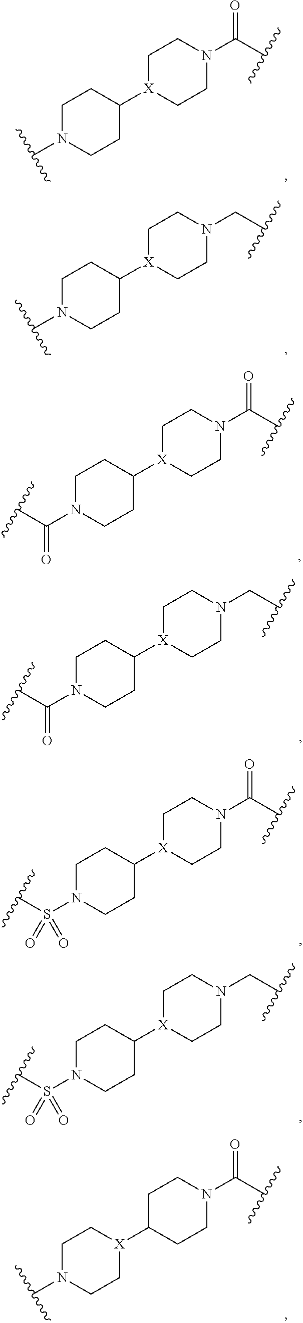

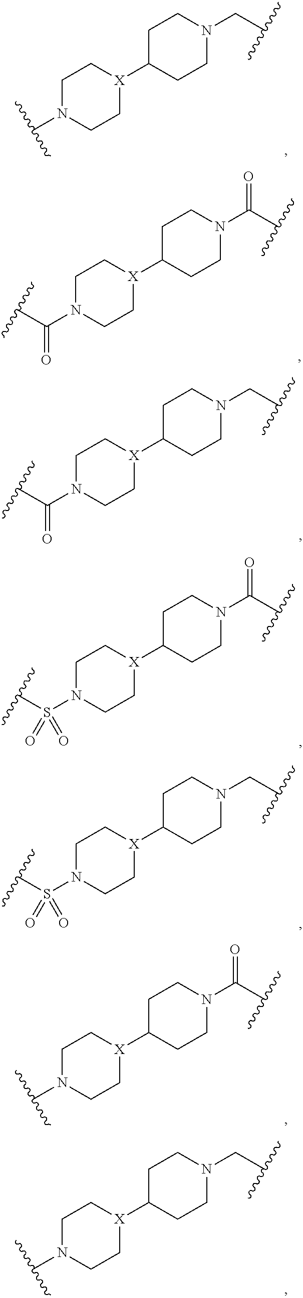

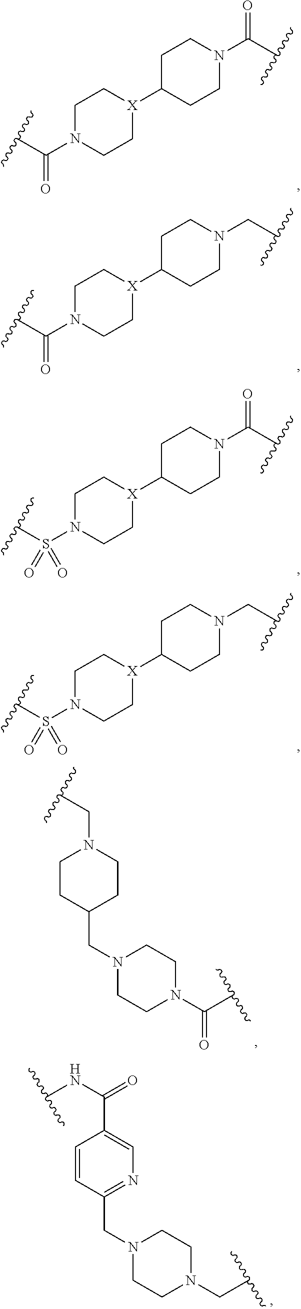

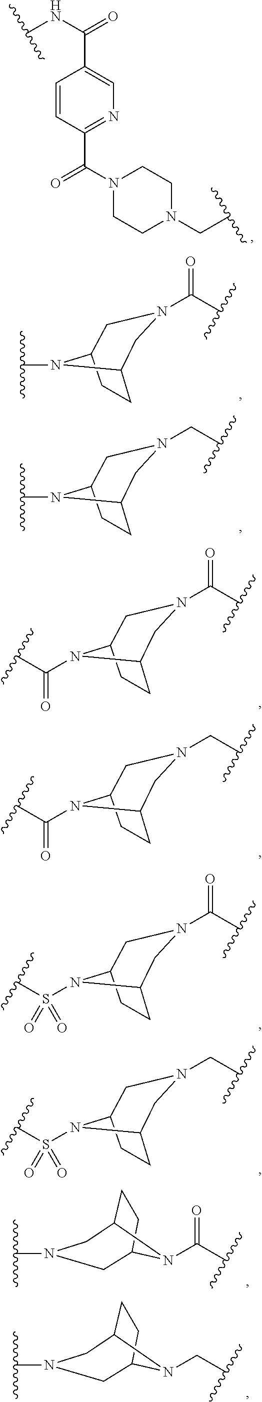

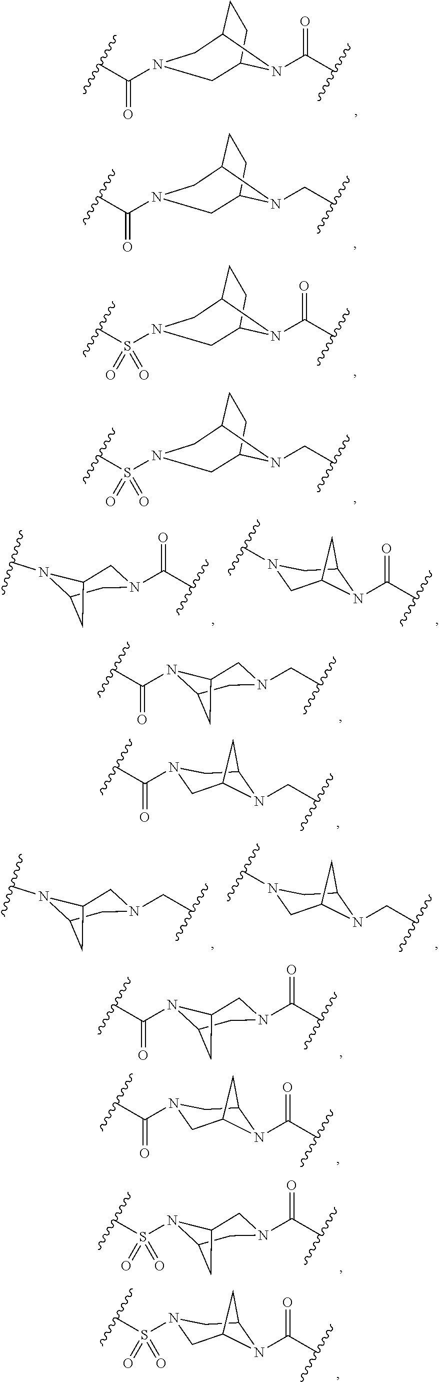

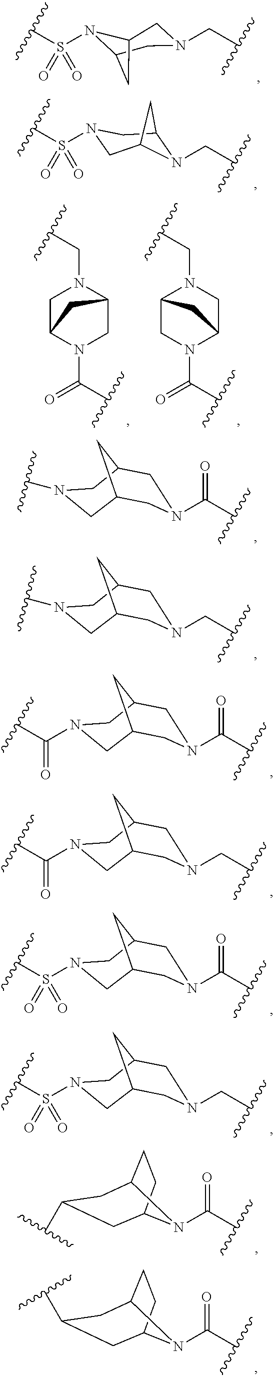

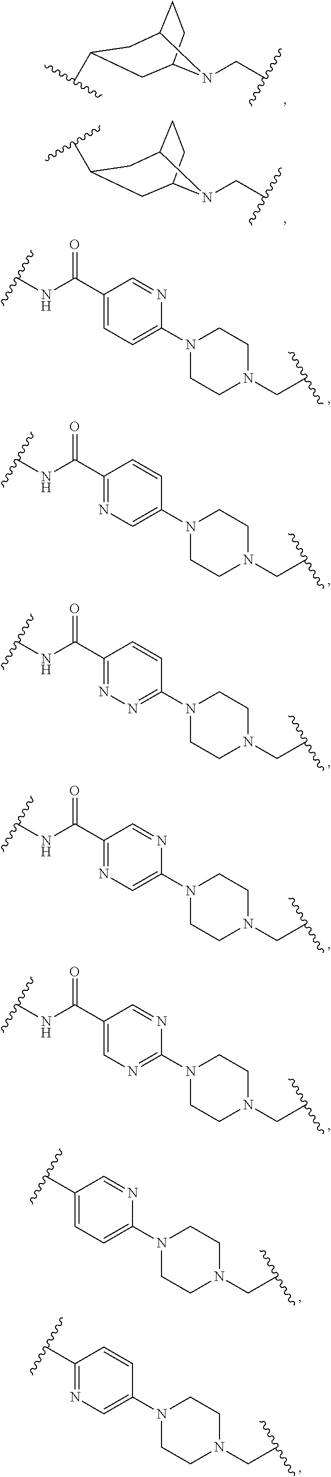

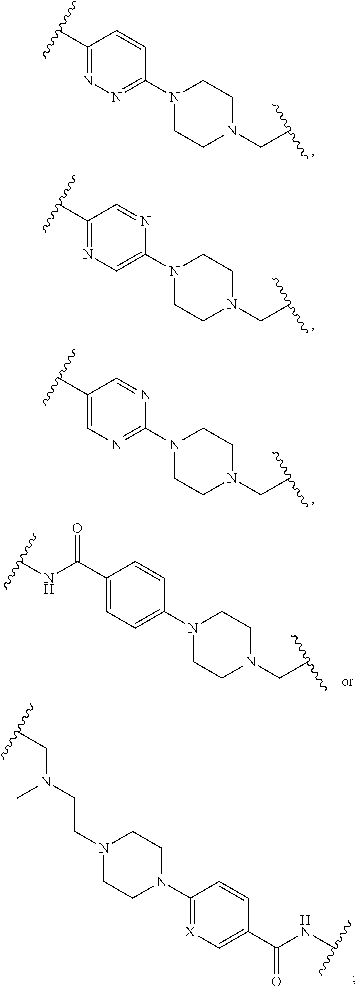

L₂ is a bond,

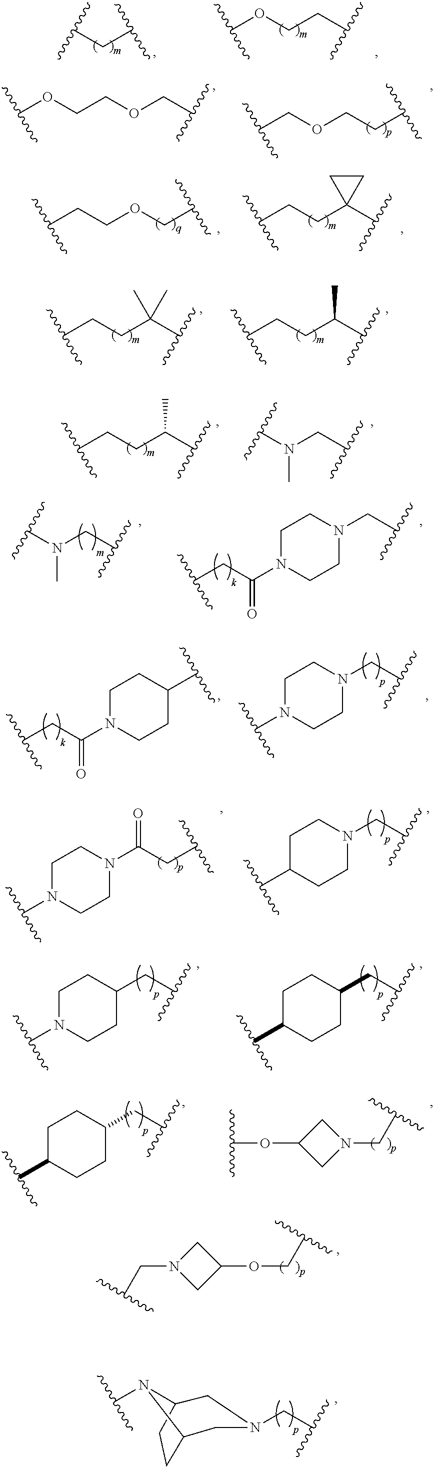

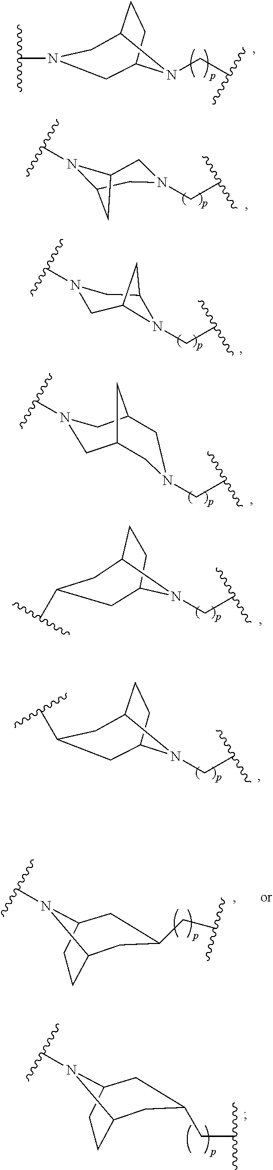

each X is independently CH or N
each k is independently 0, 1, 2, 3, 4, 5, or 6;
each m is independently 2, 3, 4, 5, 6, or 7;
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl; provided that when R₂ is SO₂CF₃ and L₁ is

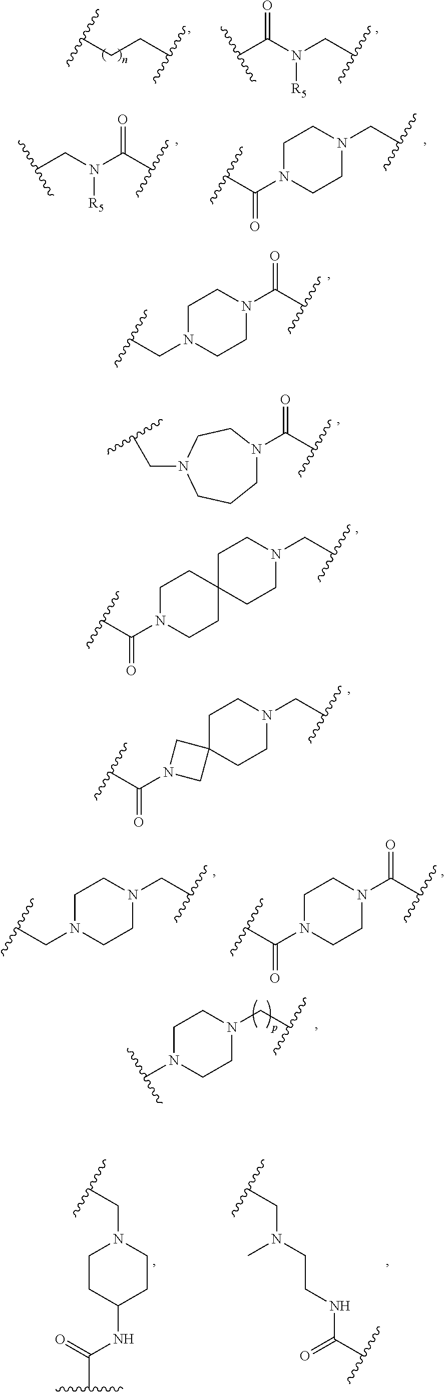

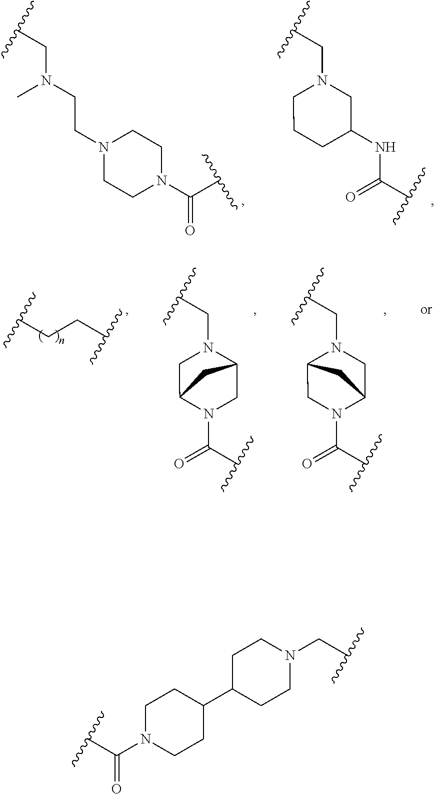

then L₂ is not a bond,

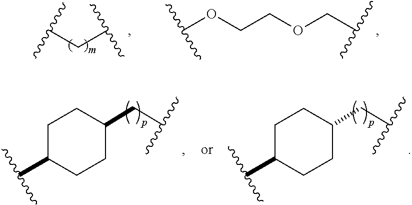

33. The compound of embodiment 32, or a pharmaceutically acceptable salt, hydrate, solvate, or prodrug thereof, wherein:
L₁ is

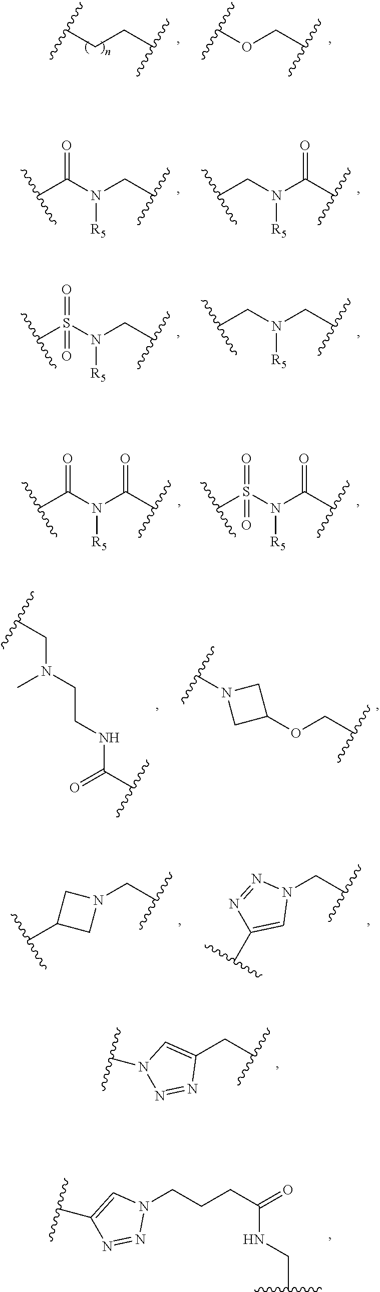

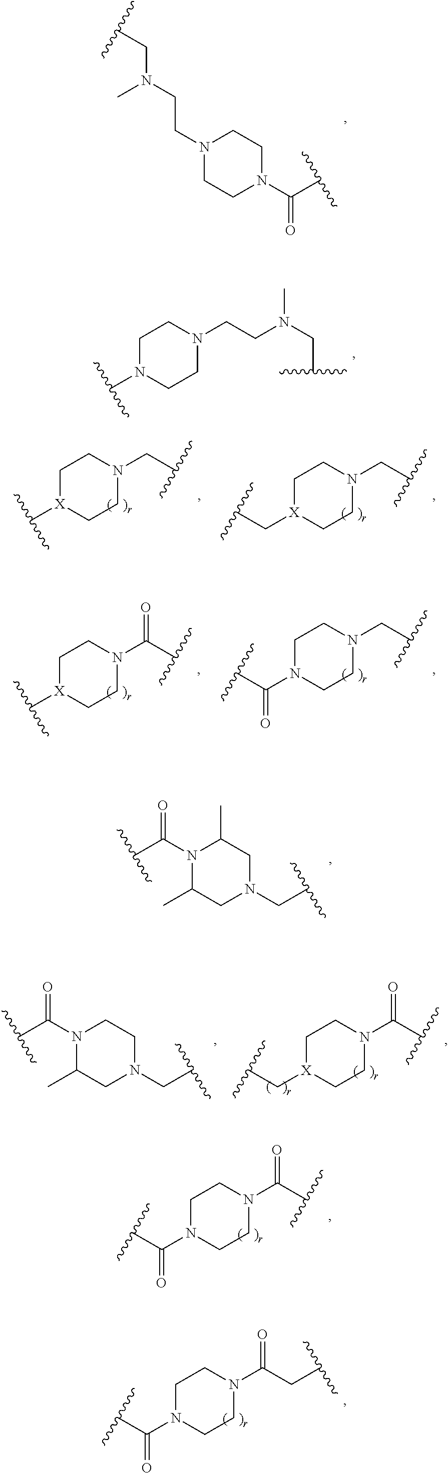

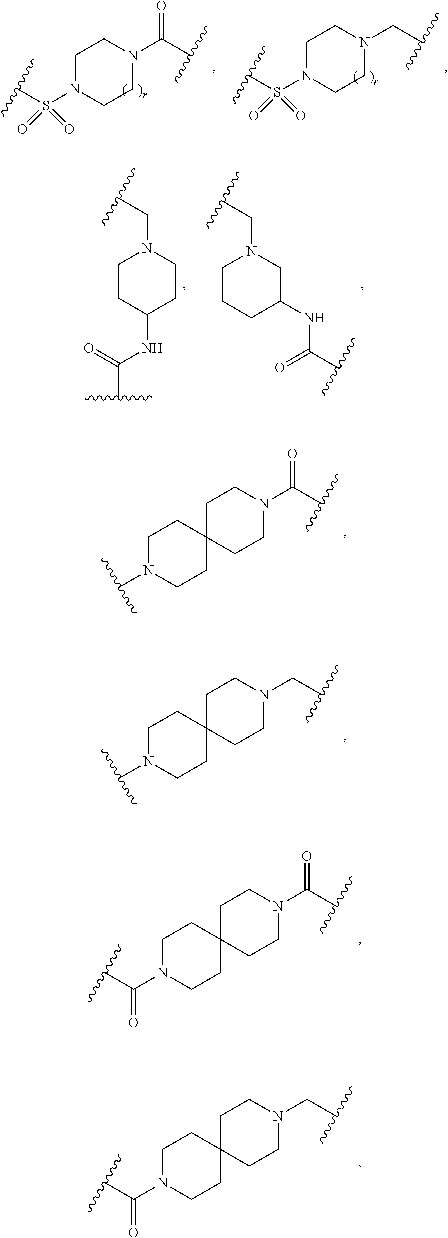

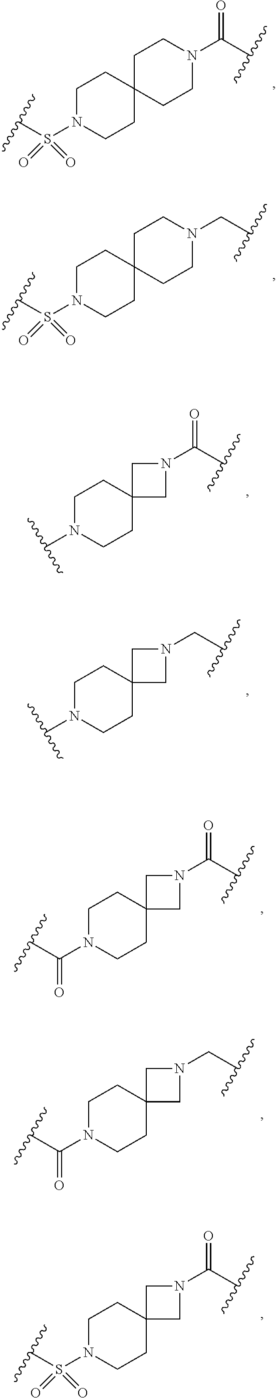

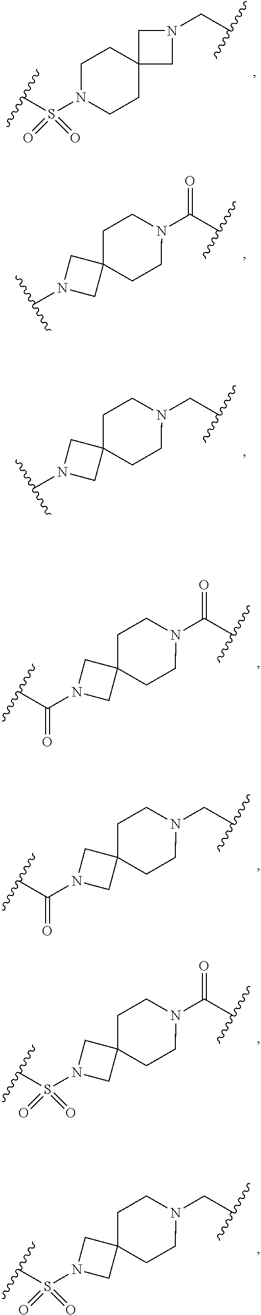

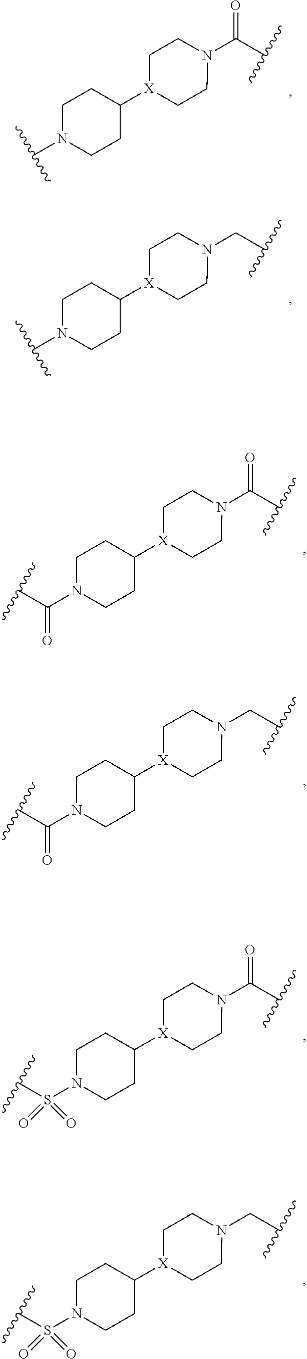

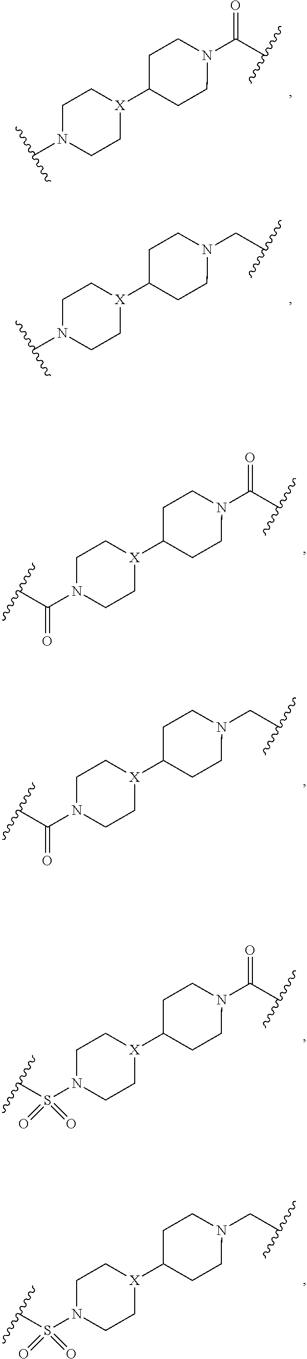

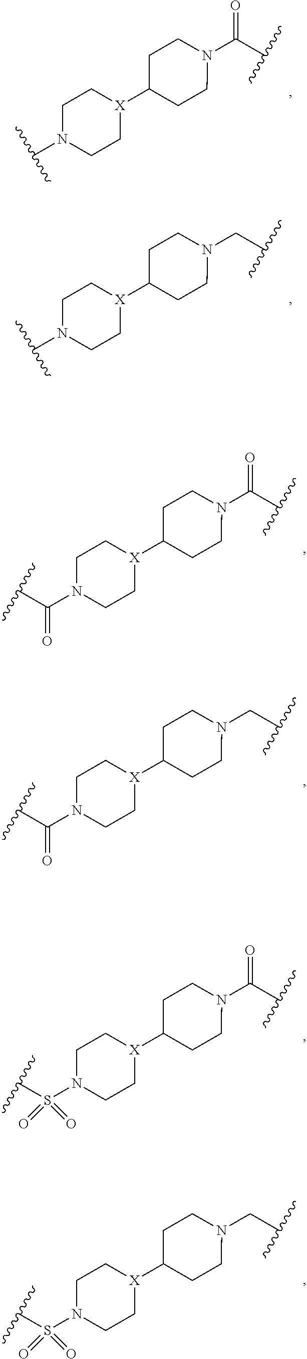

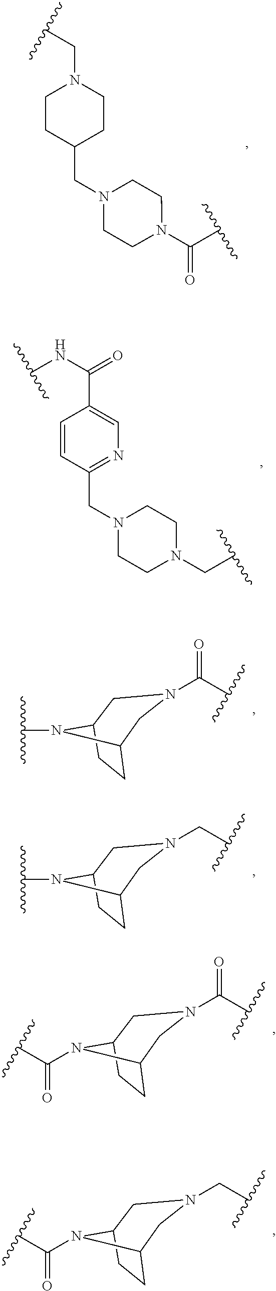 [SMILES: *CN1CCC(CN2CCN(C(*)=O)CC2)CC1 ; *CN1CCN(Cc2ccc(C(=O)N*)cn2)CC1 ; *C(=O)N1CC2CCC(C1)N2* ; *CN1CC2CCC(C1)N2* ; *C(=O)N1CC2CCC(C1)N2C(*)=O ; *CN1CC2CCC(C1)N2C(*)=O]

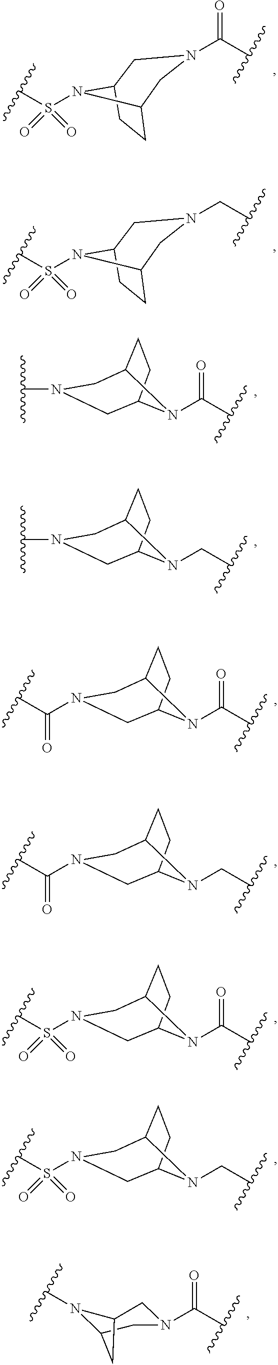

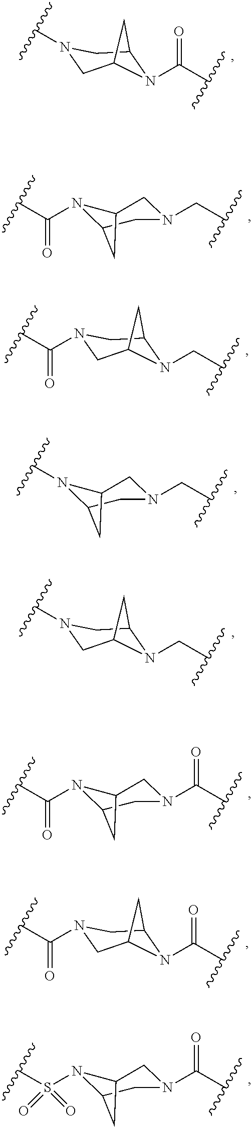

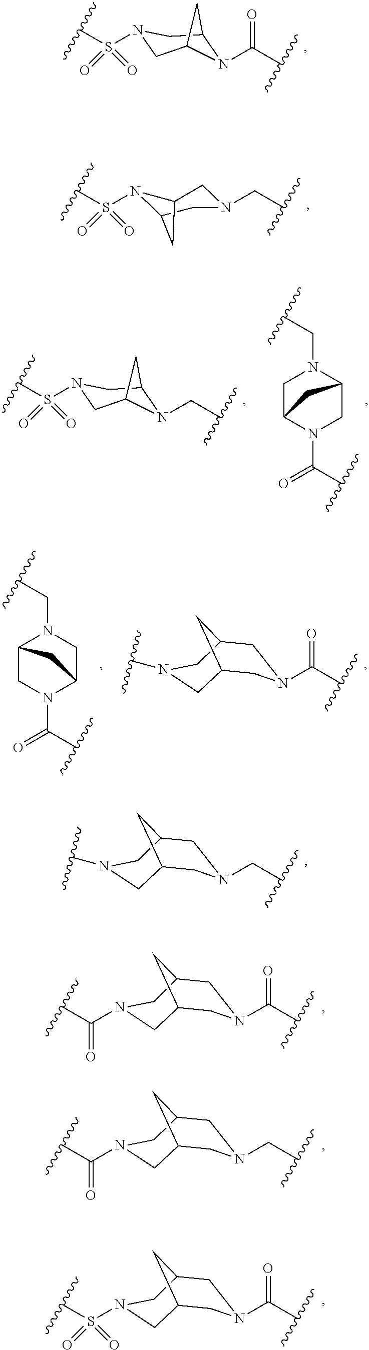

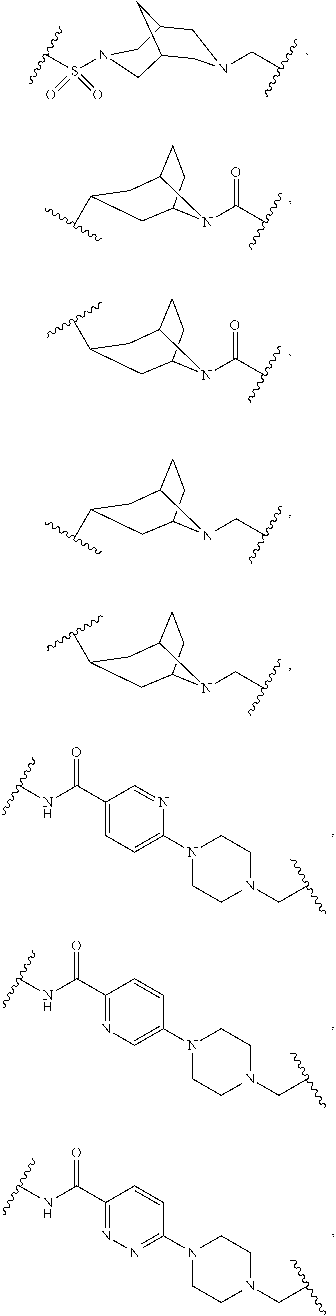

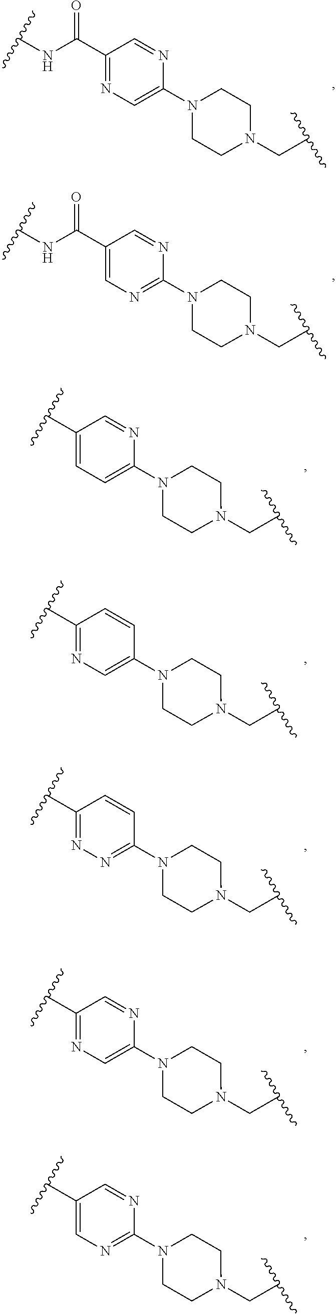

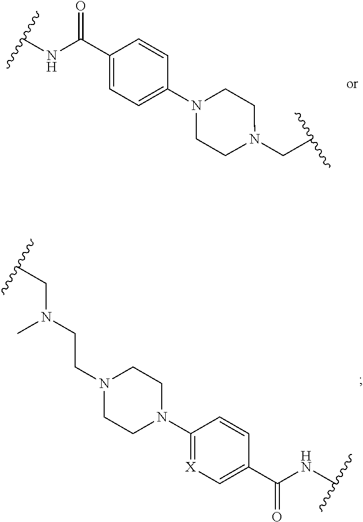

and
L₂ is a bond,

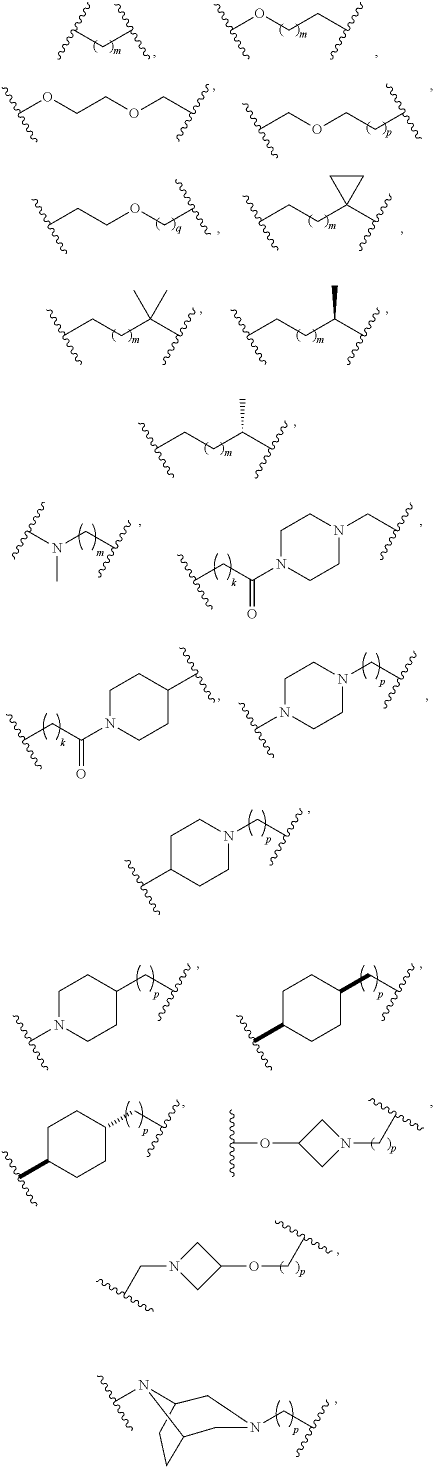

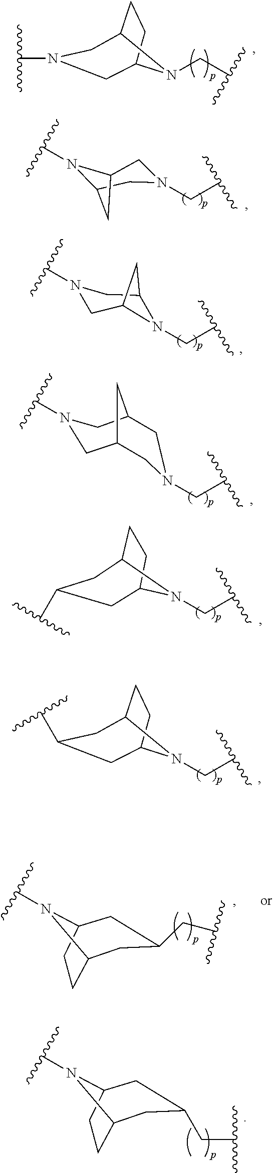

34. The compound of embodiment 32 or 33, or a pharmaceutically acceptable salt, hydrate, solvate, or prodrug thereof, wherein the compound is of formula:

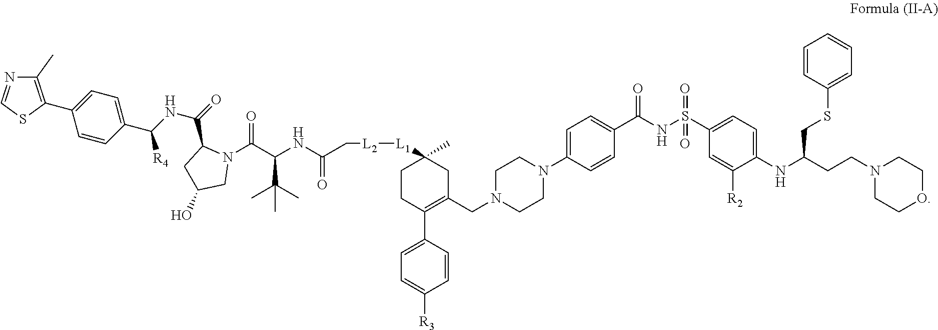

35. The compound of any one of embodiments 32-34, or a pharmaceutically acceptable salt, hydrate, solvate, or prodrug thereof, wherein L₁ is

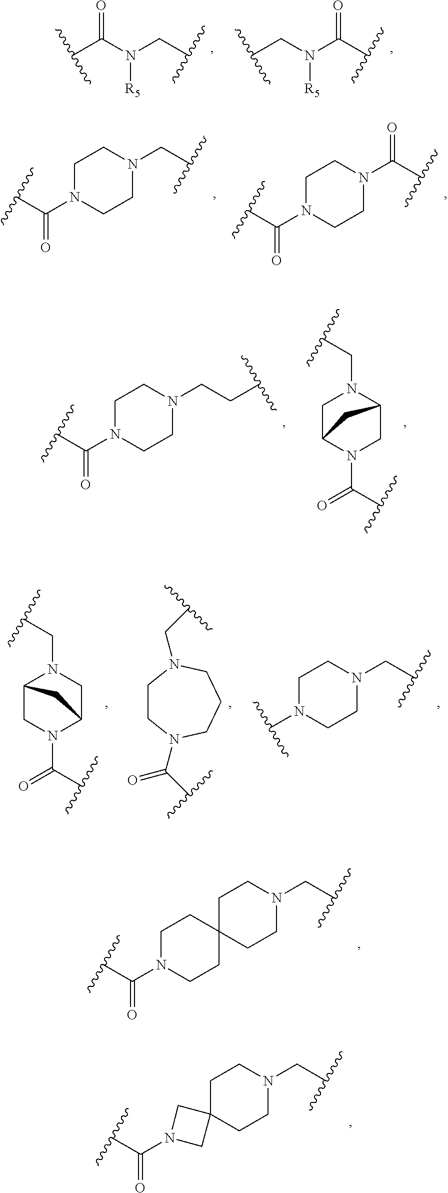

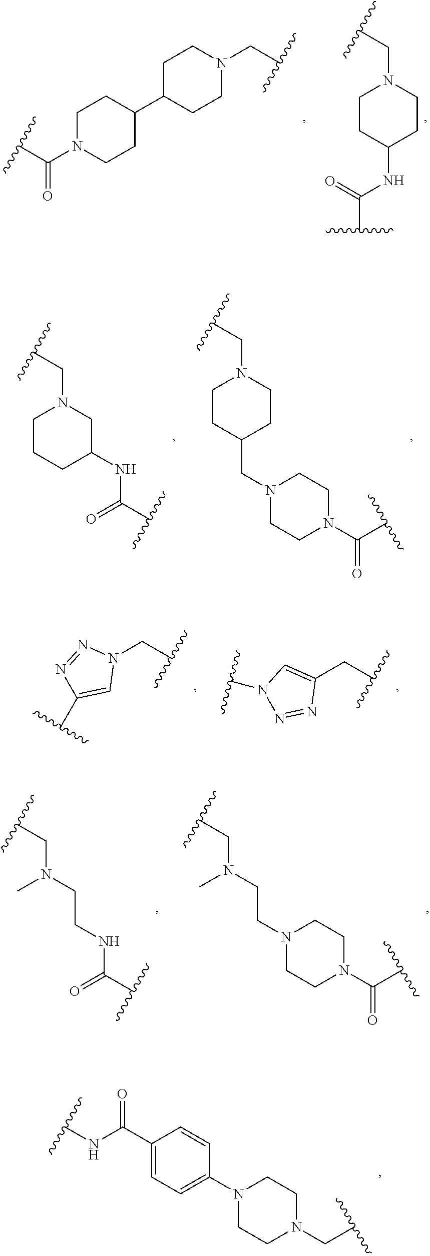

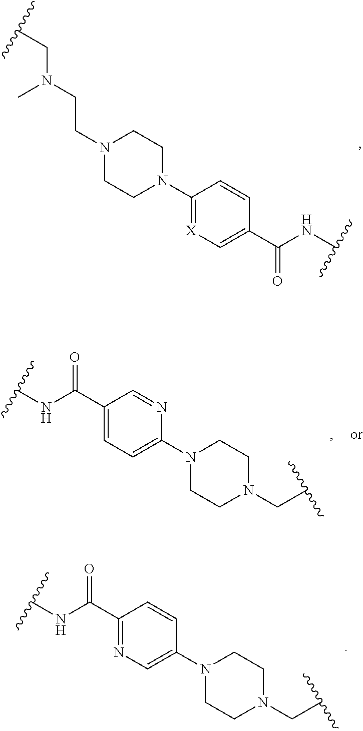

36. The compound of embodiment 35, or a pharmaceutically acceptable salt, hydrate, solvate, or prodrug thereof, wherein L₁ is

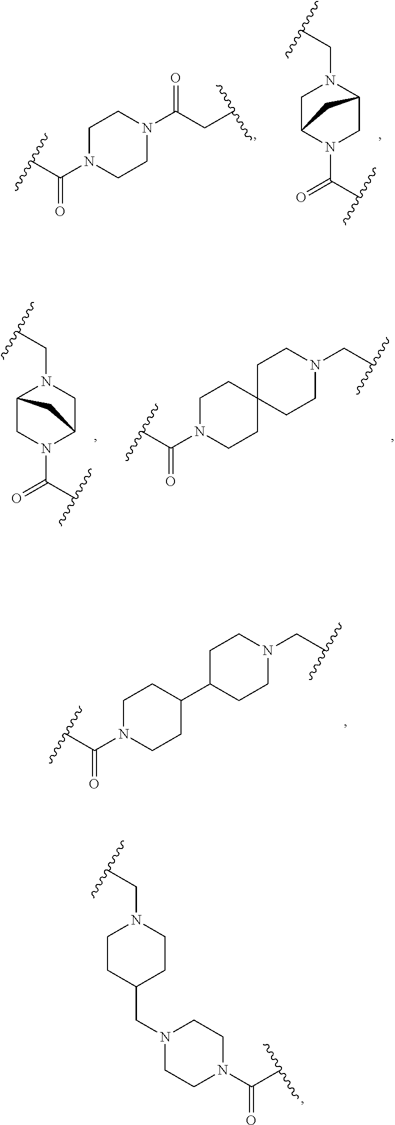 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2 ; *CN1CCC(C2CCN(C(*)=O)CC2)CC1 ; *CN1CCC(CN2CCN(C(*)=O)CC2)CC1]

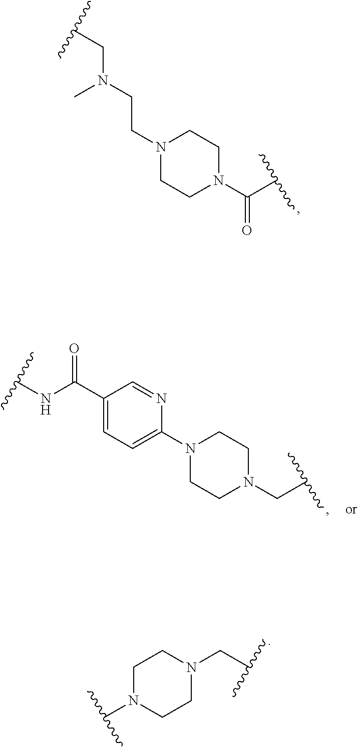 [SMILES: *CN(C)CCN1CCN(C(*)=O)CC1 ; *CN1CCN(c2ccc(C(=O)N*)cn2)CC1 ; *CN1CCN(*)CC1]

37. The compound of any one of embodiments 32-36, or a pharmaceutically acceptable salt, hydrate, solvate, or prodrug thereof, wherein L₂ is

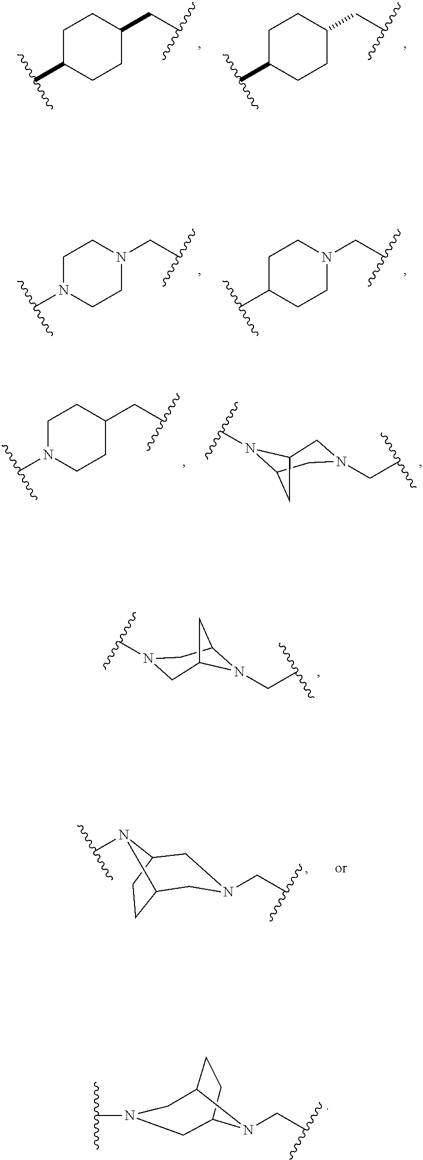

38. The compound of embodiment 37, or a pharmaceutically acceptable salt, hydrate, solvate, or prodrug thereof, wherein L₂ is

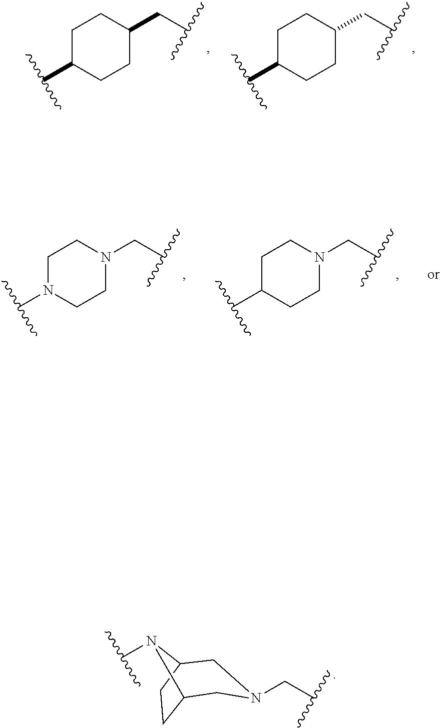 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

39. The compound of any one of embodiments 32-34 and 37-38, or a pharmaceutically acceptable salt, hydrate, solvate, or prodrug thereof, wherein L₁ is

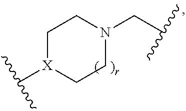

### X is N, and r is 1.

40. The compound of embodiment 32, or a pharmaceutically acceptable salt, hydrate, solvate, or prodrug thereof, wherein L₁ is

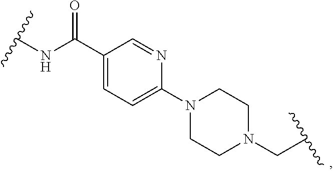 [SMILES: *CN1CCN(c2ccc(C(=O)N*)cn2)CC1]

and L₂ is a bond or

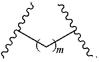

41. The compound of embodiment 32, or a pharmaceutically acceptable salt, hydrate, solvate, or prodrug thereof, wherein L₁ is

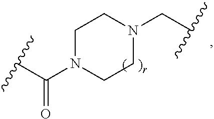

### and L₂ is

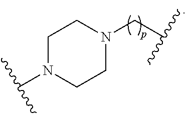

42. The compound of any one of embodiments 32-41, or a pharmaceutically acceptable salt, hydrate, solvate, or prodrug thereof, wherein R₂ is NO₂.

43. The compound of any one of embodiments 32-41, or a pharmaceutically acceptable salt, hydrate, solvate, or prodrug thereof, wherein R₂ is SO₂CF₃.

44. The compound of any one of embodiments 32-43, or a pharmaceutically acceptable salt, hydrate, solvate, or prodrug thereof, wherein R₃ is F, Cl, or CF₃.

45. The compound of embodiment 44, or a pharmaceutically acceptable salt, hydrate, solvate, or prodrug thereof, wherein R₃ is Cl.

46. The compound of any one of embodiments 32-45, or a pharmaceutically acceptable salt, hydrate, solvate, or prodrug thereof, wherein R₄ is CH₃.

47. The compound of any one of embodiments 32-45, or a pharmaceutically acceptable salt, hydrate, solvate, or prodrug thereof, wherein R₄ is H.

48. The compound of any one of embodiments 32-47, or a pharmaceutically acceptable salt, hydrate, solvate, or prodrug thereof, wherein R₅ is H.

49. The compound of any one of embodiments 32-47, or a pharmaceutically acceptable salt, hydrate, solvate, or prodrug thereof, wherein R₅ is optionally substituted C₁₋₆ alkyl.

50. The compound of embodiment 49, or a pharmaceutically acceptable salt, hydrate, solvate, or prodrug thereof, wherein R₅ is CH₃.

51. The compound of any one of embodiments 32-50, or a pharmaceutically acceptable salt, hydrate, solvate, or prodrug thereof, wherein X is N.

52. The compound of any one of embodiments 32-50, or a pharmaceutically acceptable salt, hydrate, solvate, or prodrug thereof, wherein X is CH.

53. The compound of any one of embodiments 32-52, or a pharmaceutically acceptable salt, hydrate, solvate, or prodrug thereof, wherein r is 1.

54. The compound of any one of embodiments 32-52, or a pharmaceutically acceptable salt, hydrate, solvate, or prodrug thereof, wherein r is 2.

55. The compound of embodiment 32, or a pharmaceutically acceptable salt, hydrate, solvate, or prodrug thereof, wherein the compound is:

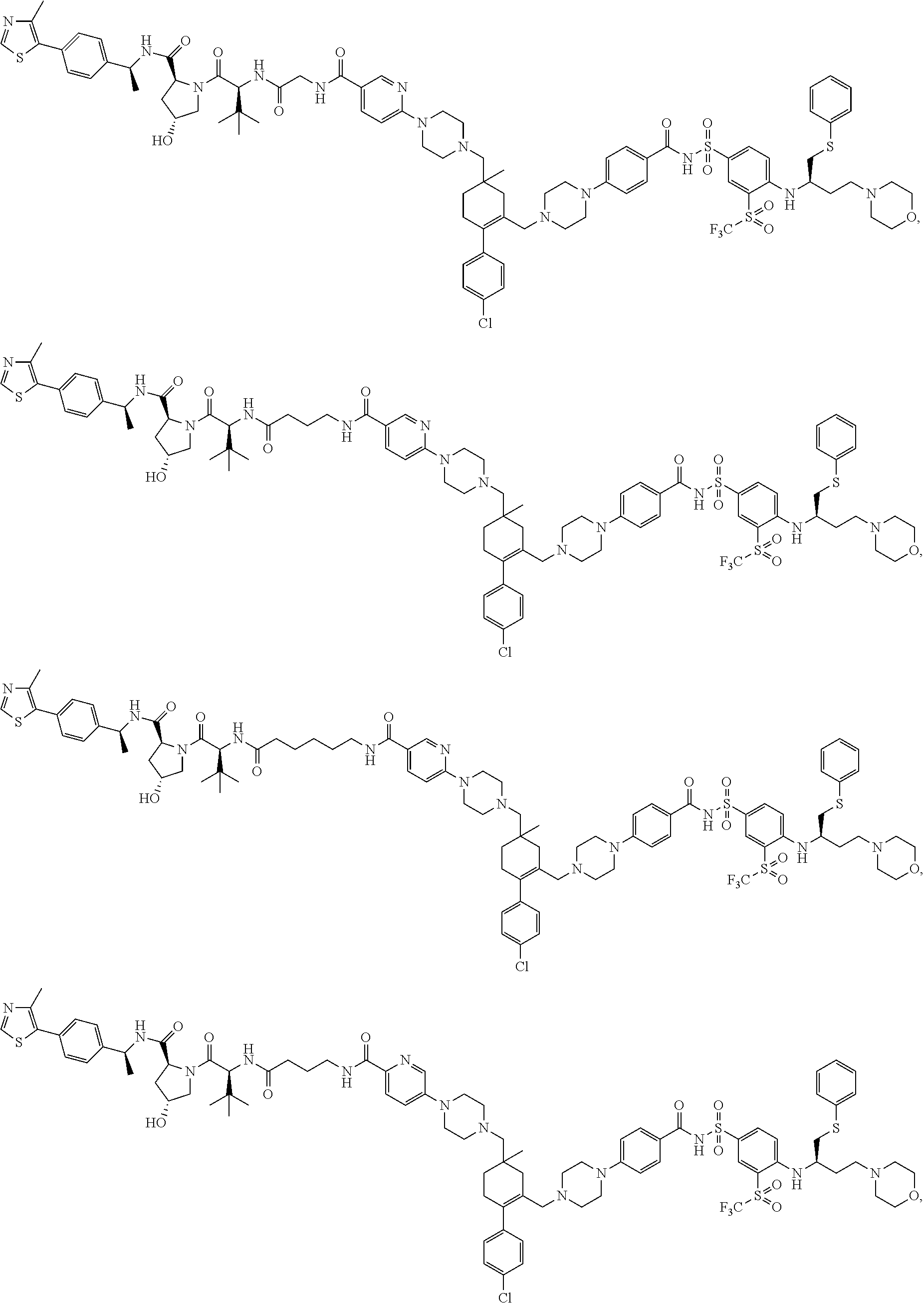

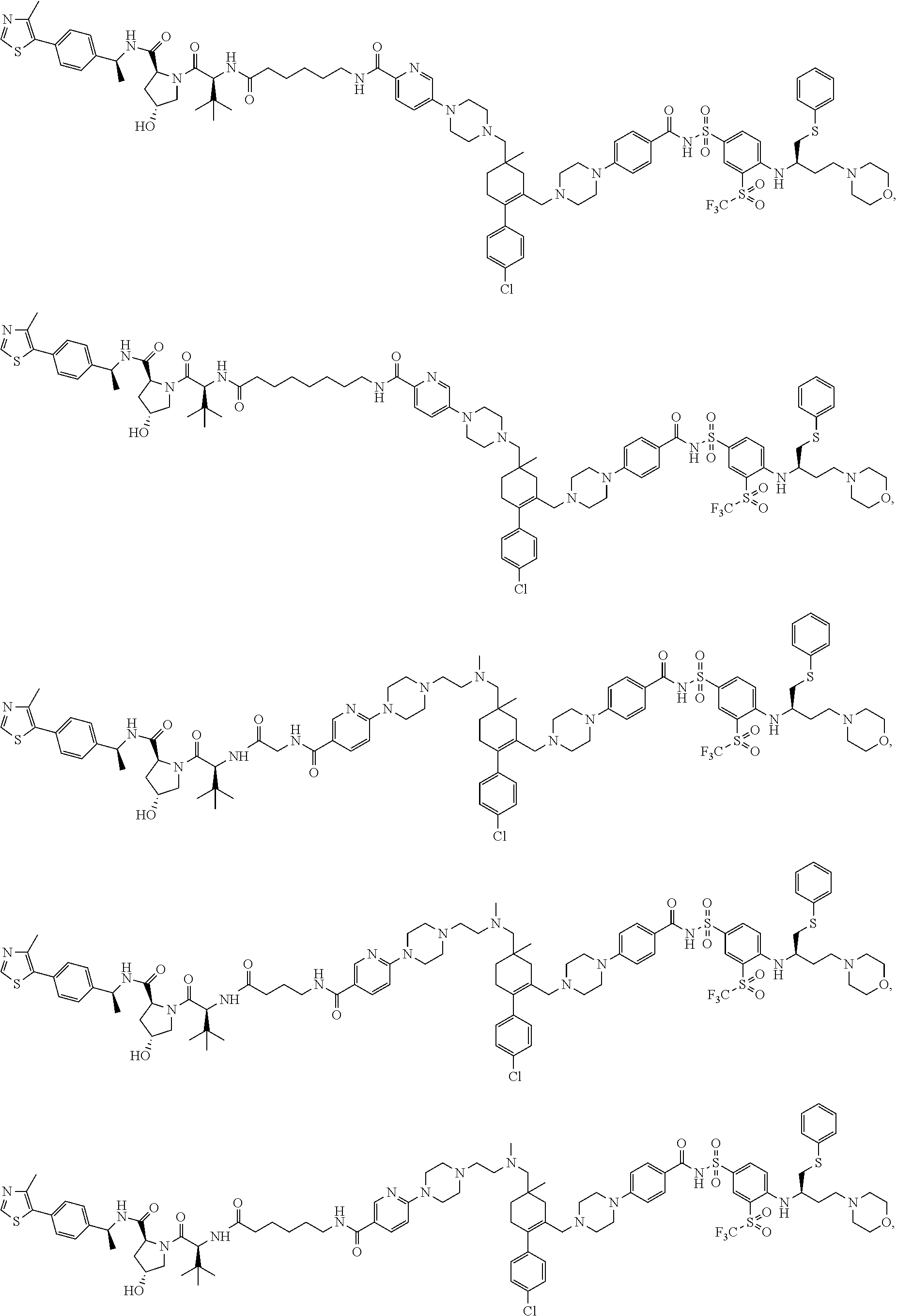

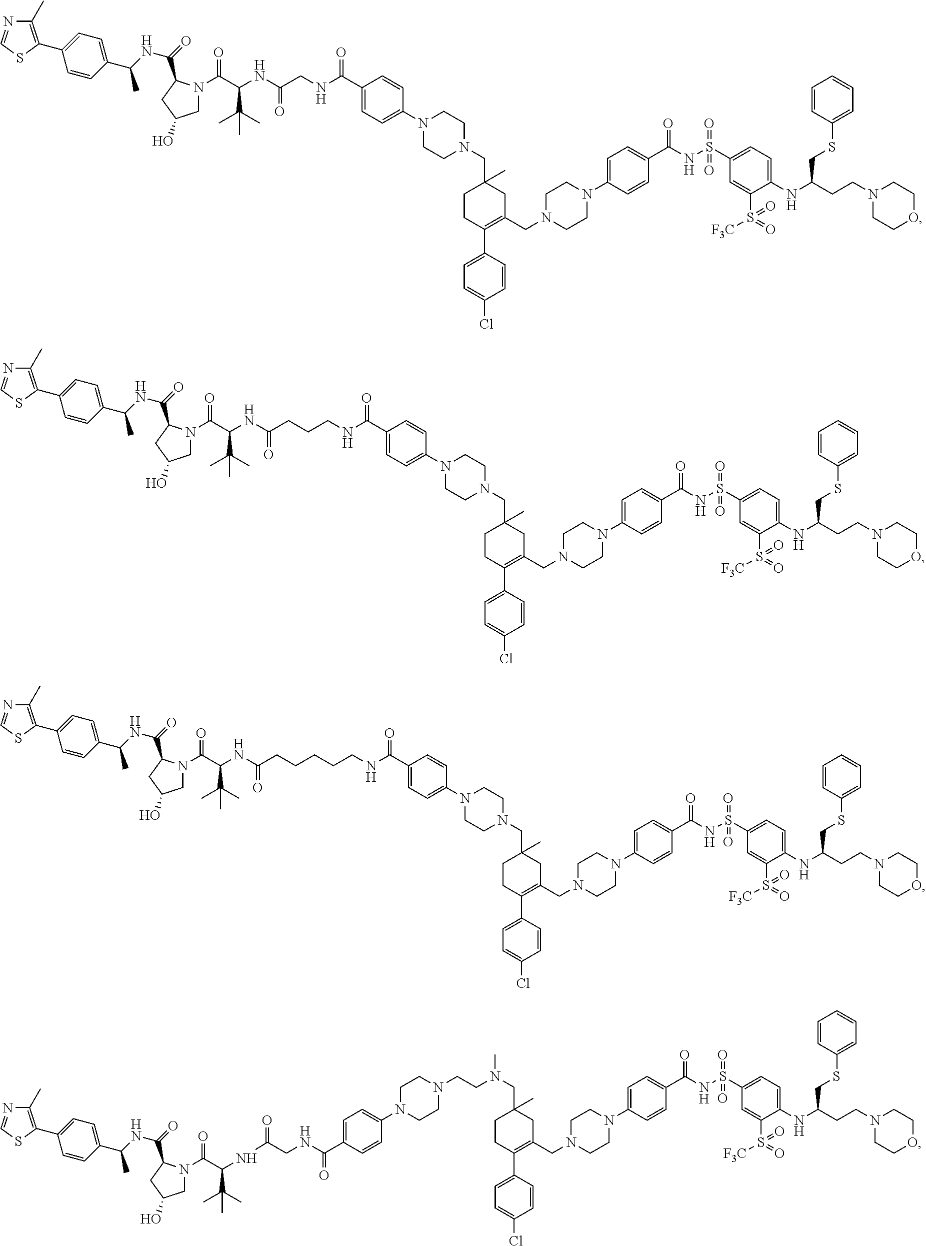

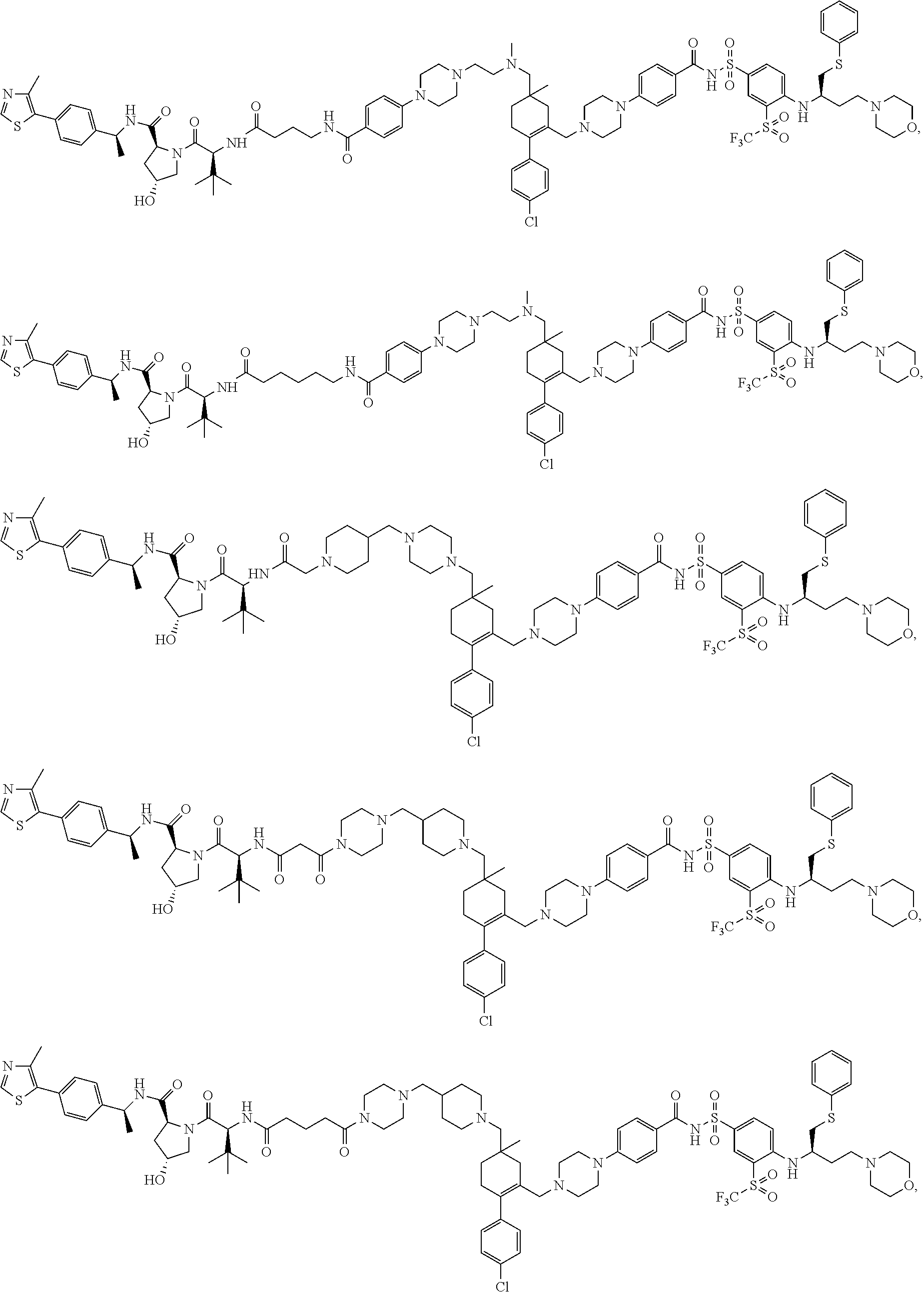

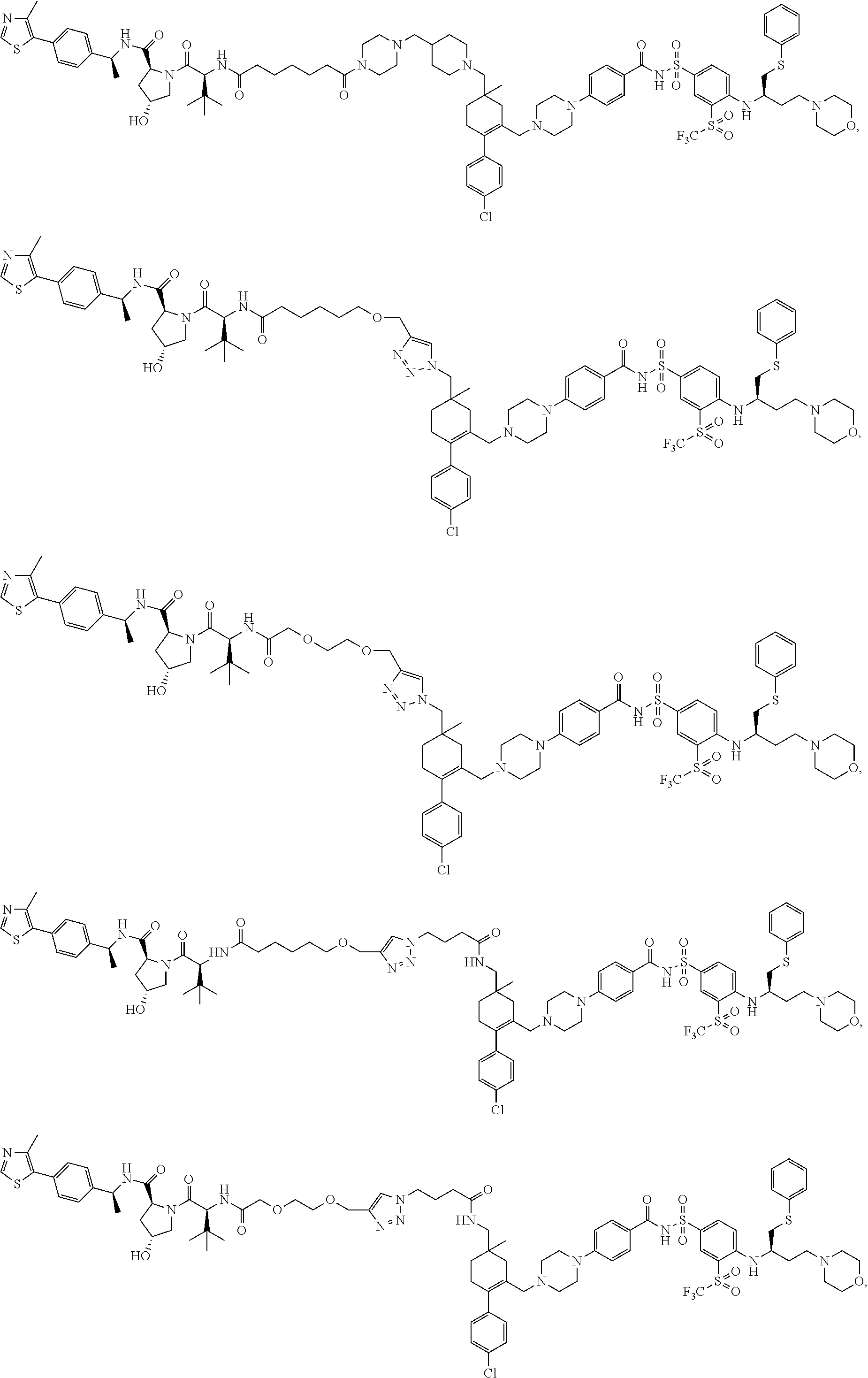

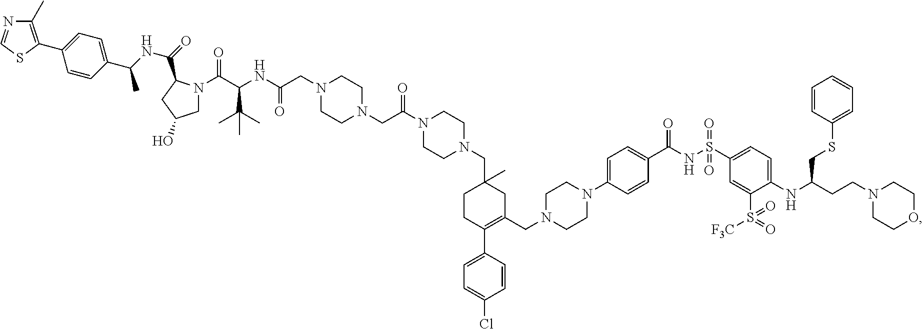 [SMILES: Cc1ncsc1-c1ccc([C@H](C)NC(=O)[C@@H]2C[C@@H](O)CN2C(=O)[C@@H](NC(=O)CN2CCN(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1]

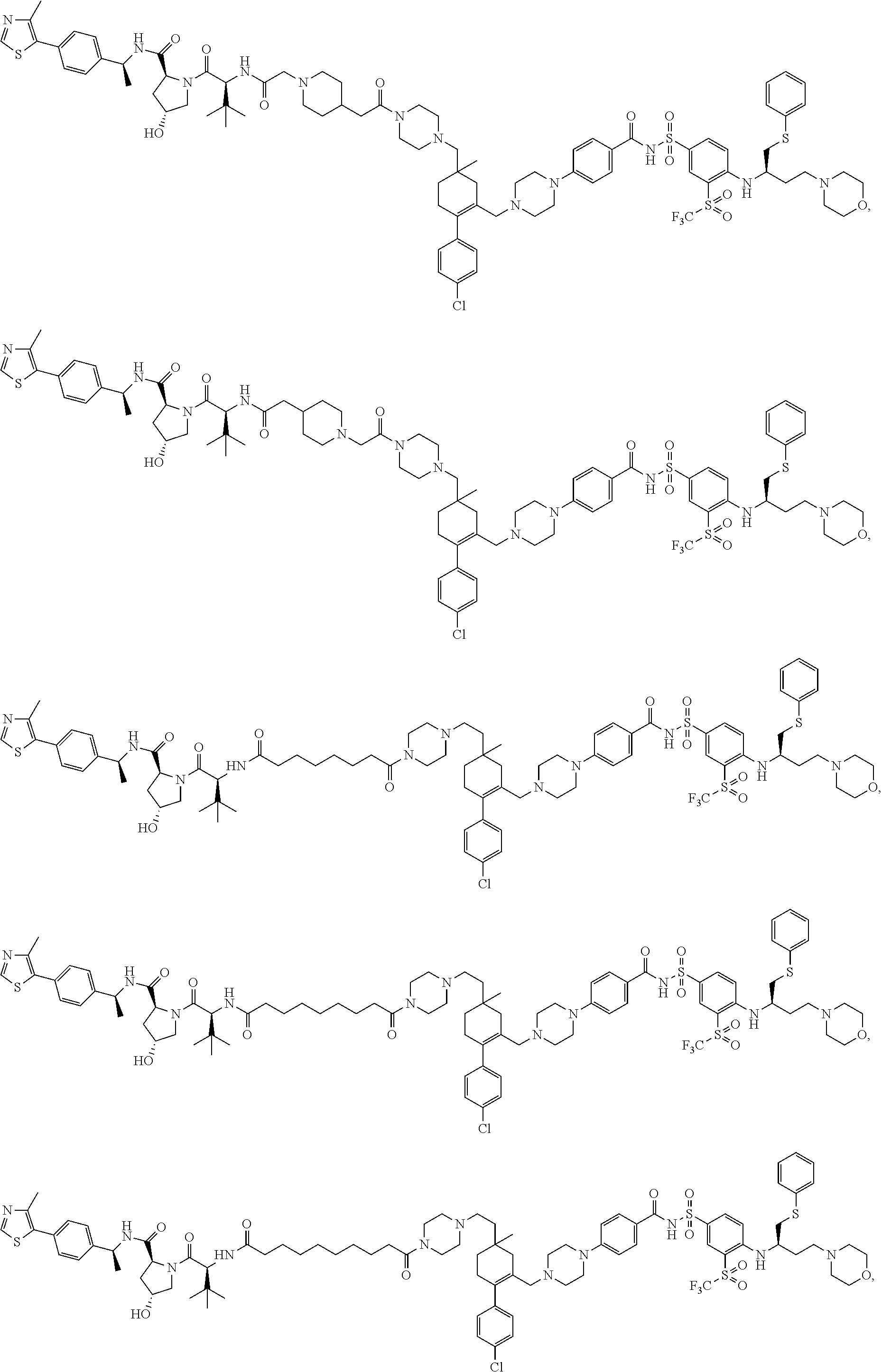

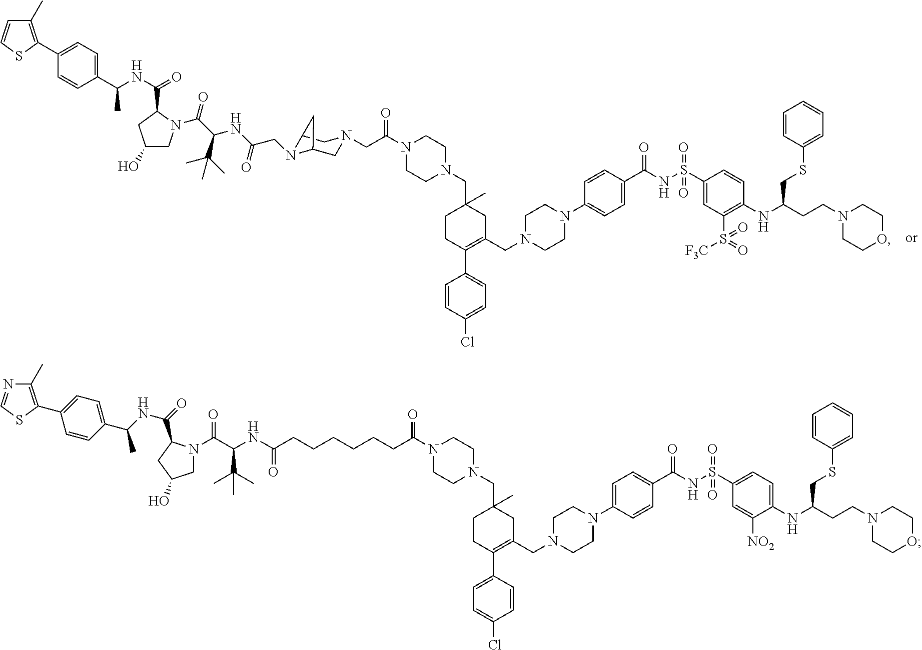 [SMILES: Cc1ccsc1-c1ccc([C@H](C)NC(=O)[C@@H]2C[C@@H](O)CN2C(=O)[C@@H](NC(=O)CN2C3CC2CN(CC(=O)N2CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC2)C3)C(C)(C)C)cc1 ; "or" ; Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c([N+](=O)[O-])c6)cc5)CC4)C3)CC2)C(C)(C)C)cc1]

or a pharmaceutically acceptable salt, hydrate, solvate, or prodrug thereof.

56. The compound of embodiment 32, or a pharmaceutically acceptable salt, hydrate, solvate, or prodrug thereof, wherein the compound is:

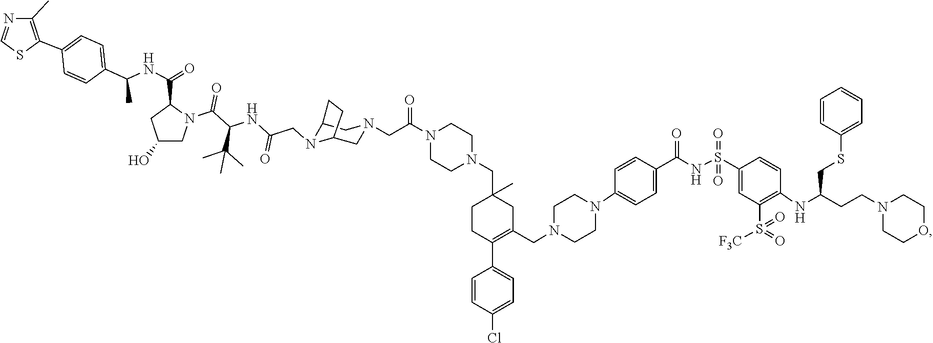 [SMILES: Cc1ncsc1-c1ccc([C@H](C)NC(=O)[C@@H]2C[C@@H](O)CN2C(=O)[C@@H](NC(=O)CN2C3CCC2CN(CC(=O)N2CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC2)C3)C(C)(C)C)cc1]

or a pharmaceutically acceptable salt, hydrate, solvate, or prodrug thereof.

57. A compound of the formula:

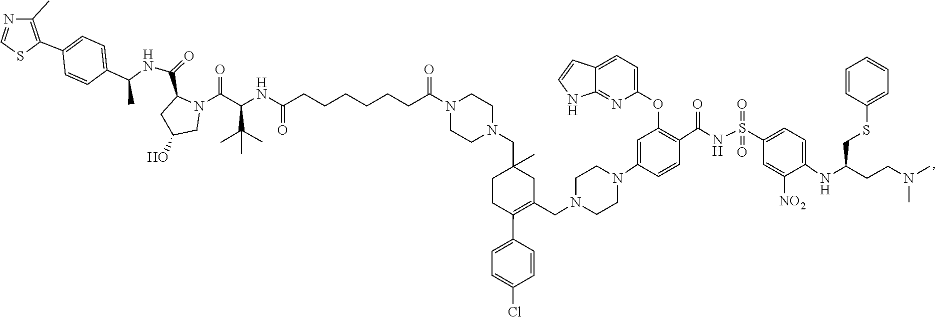 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c([N+](=O)[O-])c6)c(Oc6ccc7cc[nH]c7n6)c5)CC4)C3)CC2)C(C)(C)C)cc1]

or a pharmaceutically acceptable salt thereof.

58. A pharmaceutical composition comprising a compound of any one of embodiments 1-57, or a pharmaceutically acceptable salt, hydrate, solvate, or prodrug thereof, and a pharmaceutically acceptable carrier.

59. The pharmaceutical composition of embodiment 58, further comprising an additional agent.

60. The pharmaceutical composition of embodiment 59, wherein the additional agent is an anti-cancer agent.

61. The pharmaceutical composition of embodiment 60, wherein the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

62. A method of degrading Bcl-2 proteins, the method comprising administering an effective amount of a compound of any one of embodiments 1-57, or a pharmaceutically acceptable salt, hydrate, solvate, or prodrug thereof.

63. The method of embodiment 62, wherein the compound is administered in vitro.

64. The method of embodiment 62, wherein the compound is administered in vivo.

65. The method of embodiment 62, further comprising administering the compound to a subject.

66. A method of treating a disease or disorder in a subject in need thereof, the method comprising administering an effective amount of a compound of any one of embodiments 1-57, or a pharmaceutically acceptable salt, hydrate, solvate, or prodrug thereof.

67. A method of treating a subject suffering from or susceptible to a disease or disorder, the method comprising administering an effective amount of a compound of any one of embodiments 1-57, or a pharmaceutically acceptable salt, hydrate, solvate, or prodrug thereof.

68. The method of embodiment 66 or 67, wherein the disease is cancer.

69. The method of embodiment 68, wherein the cancer is a solid tumor.

70. The method of embodiment 68, wherein the cancer is chronic lymphocytic leukemia.

71. The method of embodiment 68, wherein the cancer is acute lymphoblastic leukemia.

72. A method of treating a Bcl-2-mediated cancer in a subject in need thereof, the method comprising administering an effective amount of a compound of any one of embodiments 1-57, or a pharmaceutically acceptable salt, hydrate, solvate, or prodrug thereof, wherein the platelet toxicity of the compound is less than that of other Bcl-2 inhibitors.

73. A method of treating a subject suffering from or susceptible to a Bcl-2-mediated cancer, the method comprising administering an effective amount of a compound of any one of embodiments 1-57, or a pharmaceutically acceptable salt, hydrate, solvate, or prodrug thereof, wherein the platelet toxicity of the compound is less than that of other Bcl-2 inhibitors.

74. The method of embodiment 72 or 73, wherein the Bcl-2-mediated cancer is chronic lymphocytic leukemia.

75. The method of embodiment 72 or 73, wherein the Bcl-2-mediated cancer is a solid tumor.

76. The method of embodiment 75, wherein the Bcl-2-mediated cancer is small cell lung cancer.

77. The method of any one of embodiments 72-76, wherein the other Bcl-2 inhibitor is venetoclax or ABT-263.

78. A method of treating a Bcl-2-mediated cancer in a subject in need thereof, the method comprising administering an effective amount of a compound of any one of embodiments 1-57, or a pharmaceutically acceptable salt, hydrate, solvate, or prodrug thereof, wherein the ratio of human platelet toxicity (IC₅₀) to anticancer activity (IC₅₀) of the compound is greater than one.

79. A method of treating a subject suffering from or susceptible to a Bcl-2-mediated cancer, the method comprising administering an effective amount of a compound of any one of embodiments 1-57, or a pharmaceutically acceptable salt, hydrate, solvate, or prodrug thereof, wherein the ratio of human platelet toxicity (IC₅₀) to anticancer activity (IC₅₀) of the compound is greater than one.

80. The method of embodiment 78 or 79, wherein the Bcl-2-mediated cancer is chronic lymphocytic leukemia.

81. The method of embodiment 78 or 79, wherein the Bcl-2-mediated cancer is a solid tumor.

82. The method of embodiment 81, wherein the Bcl-2-mediated cancer is small cell lung cancer.

83. The method of any one of embodiments 78-82, wherein the anticancer activity is measured in MOLT-4 cells.

84. The method of any one of embodiments 78-82, wherein the anticancer activity is measured in RS4 cells.

85. The method of any one of embodiments 78-84, wherein the anticancer activity is higher in MOLT-4 cells than in RS4 cells.

86. The method of any one of embodiments 78-84, wherein the anticancer activity is higher in RS4 cells than in MOLT-4 cells.

87. The method of any one of embodiments 78-86, wherein the ratio is greater than 2.5, greater than 5, greater than 10, greater than 20, greater than 40, greater than 60, greater than 80, greater than 100, greater than 150, greater than 200, greater than 250, greater than 300, greater than 350, greater than 400, greater than 450, or greater than 500.

88. The method of any one of embodiments 65-87, wherein the subject is a mammal.

89. The method of embodiment 88, wherein the subject is a human.

What is claimed is:

1. A compound of Formula (I), or a pharmaceutically acceptable salt thereof: 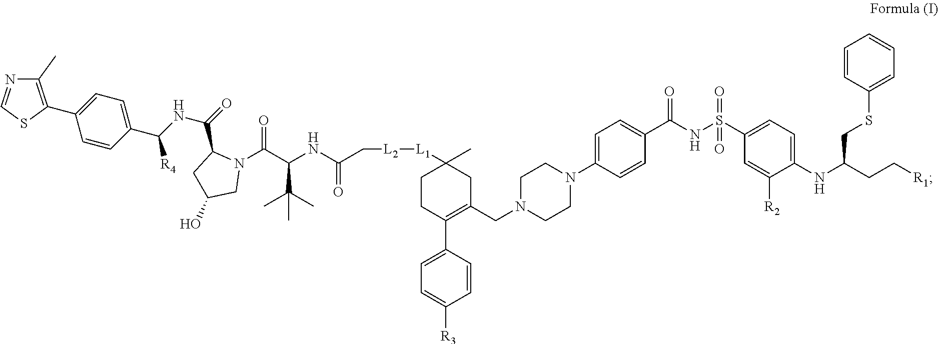
wherein R₁ is 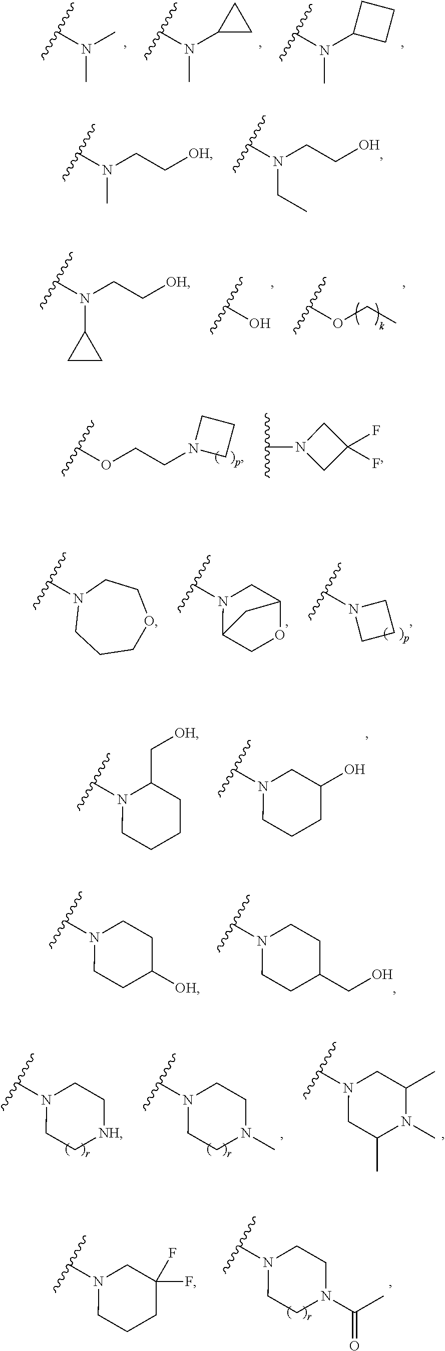 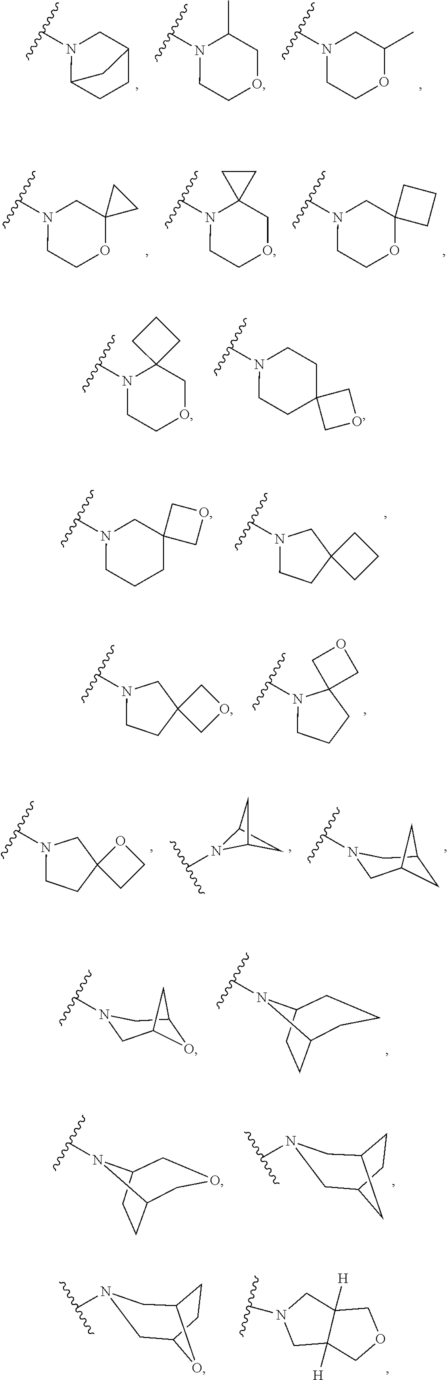 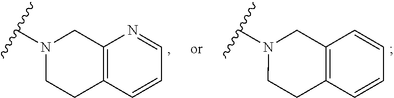 [SMILES: *N1CCc2cccnc2C1 ; *N1CCc2ccccc2C1]
R₂ is NO₂, SO₂CF₃, or SO₂CF₂Cl;
R₃ is Cl, F, CF₂H, CFH₂ or CF₃;
R₄ is H or CH₃;
L₁ is 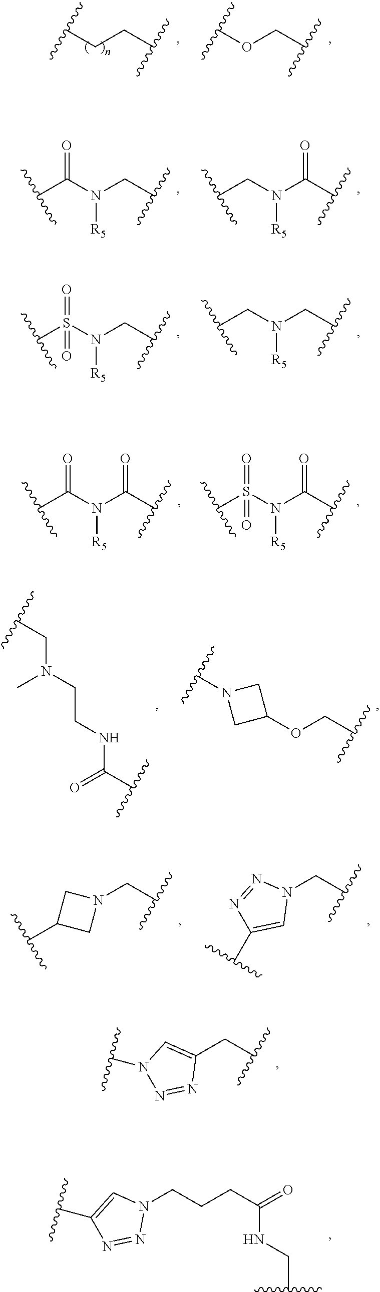 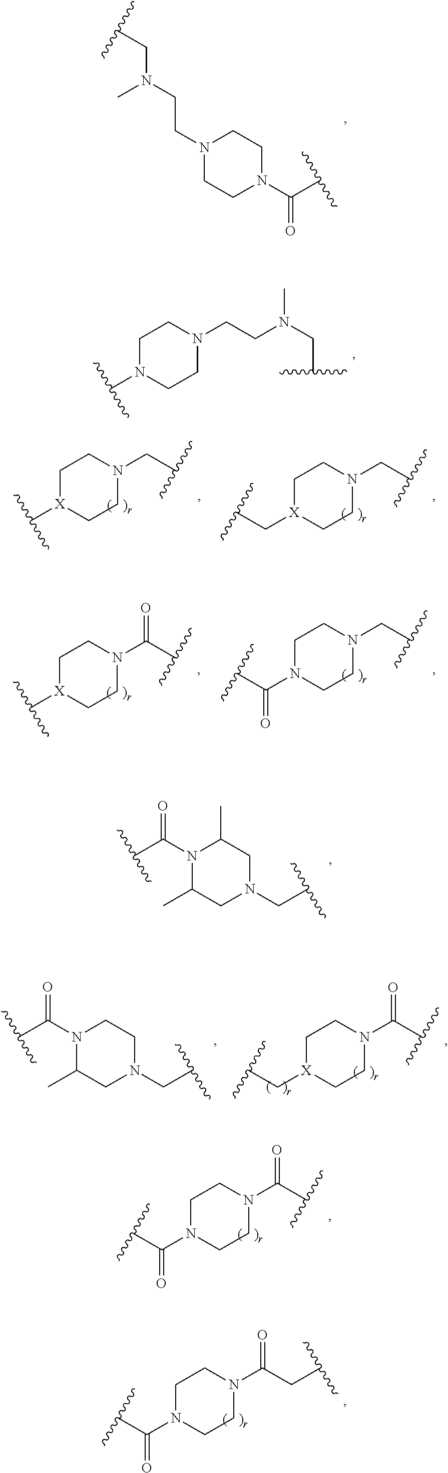 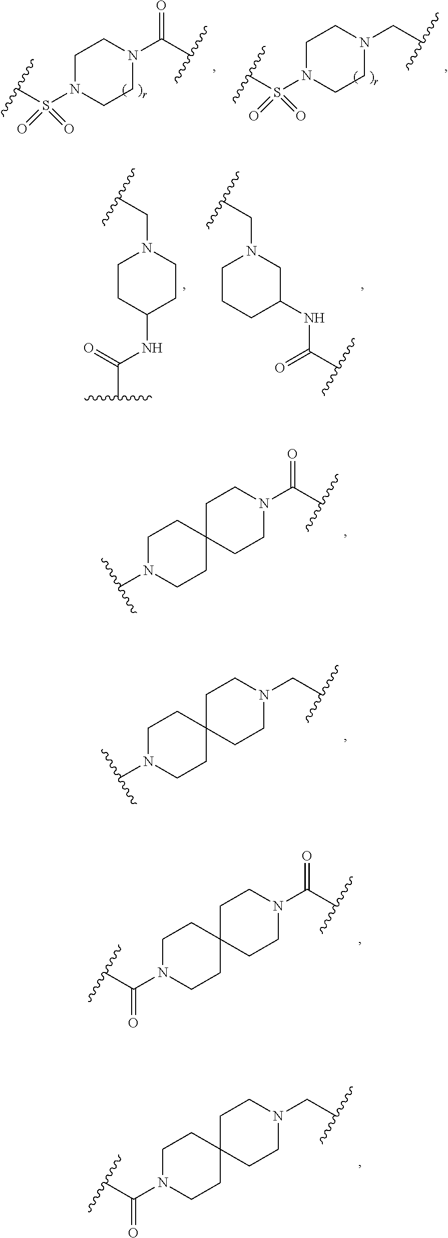 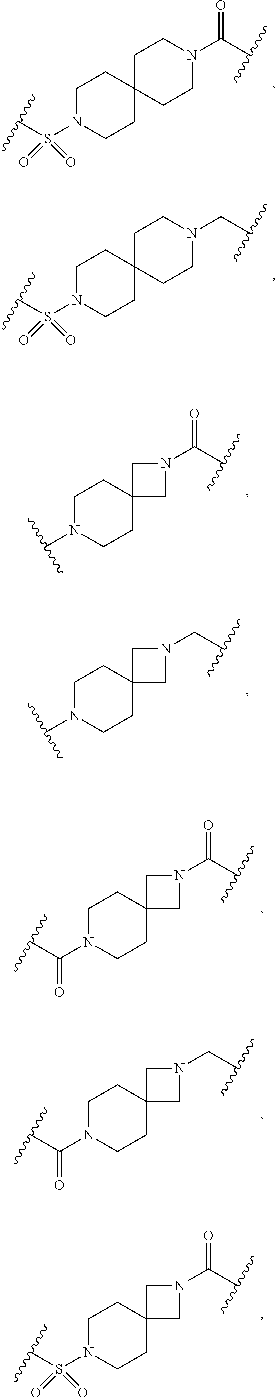 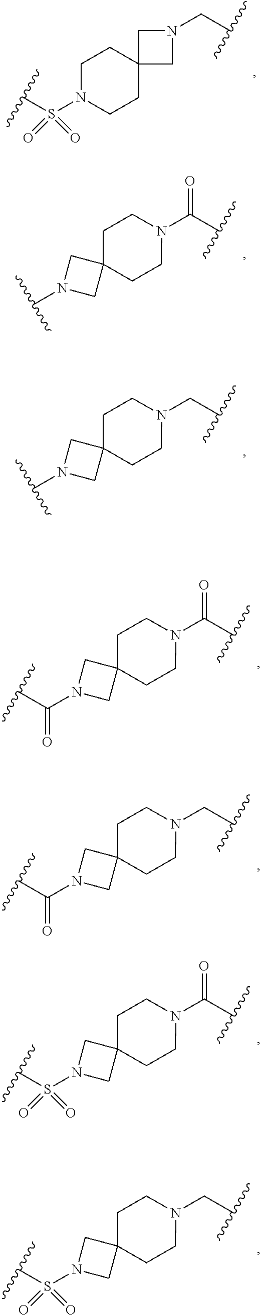 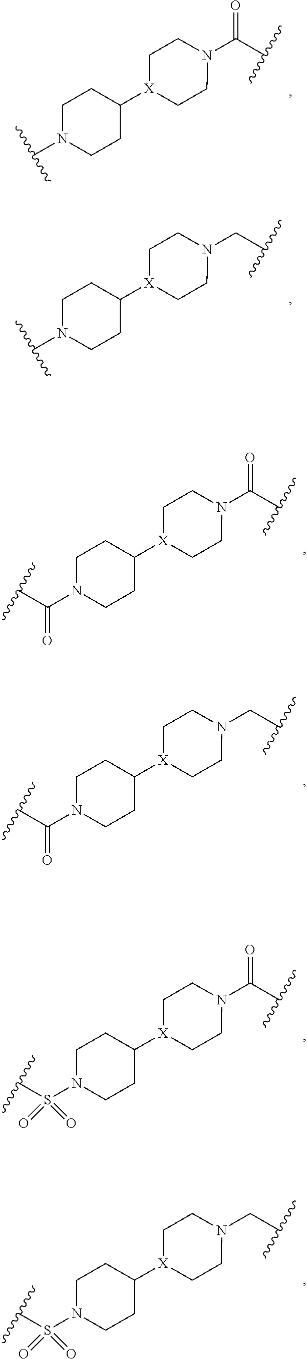 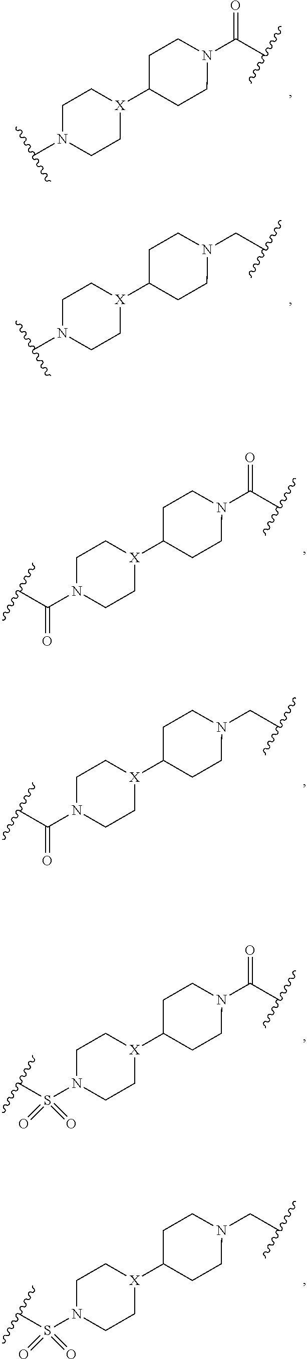 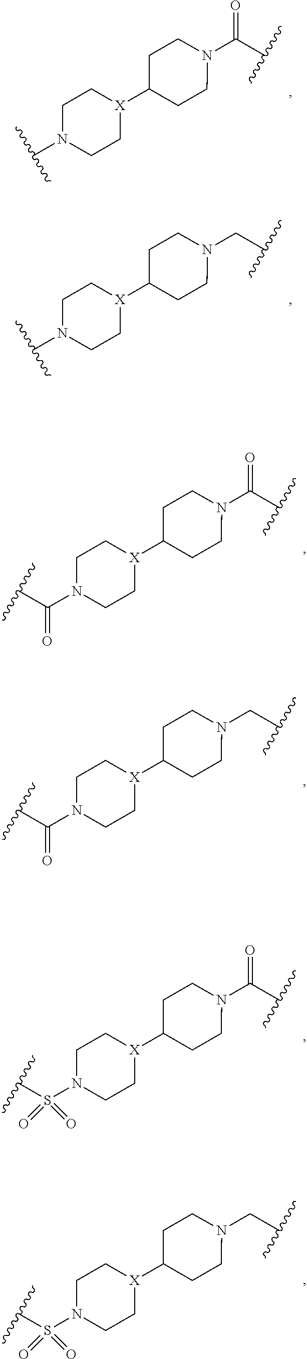 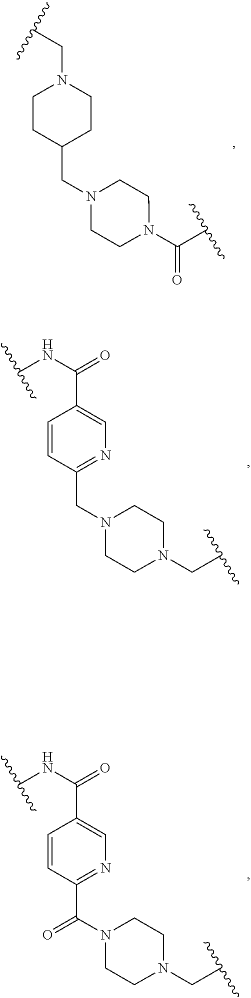 [SMILES: *CN1CCC(CN2CCN(C(*)=O)CC2)CC1 ; *CN1CCN(Cc2ccc(C(=O)N*)cn2)CC1 ; *CN1CCN(C(=O)c2ccc(C(=O)N*)cn2)CC1] 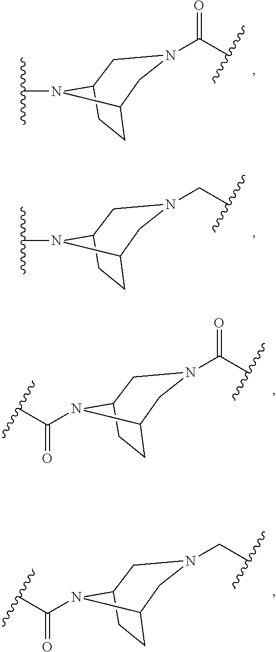 [SMILES: *C(=O)N1CC2CCC(C1)N2* ; *CN1CC2CCC(C1)N2* ; *C(=O)N1CC2CCC(C1)N2C(*)=O ; *CN1CC2CCC(C1)N2C(*)=O] 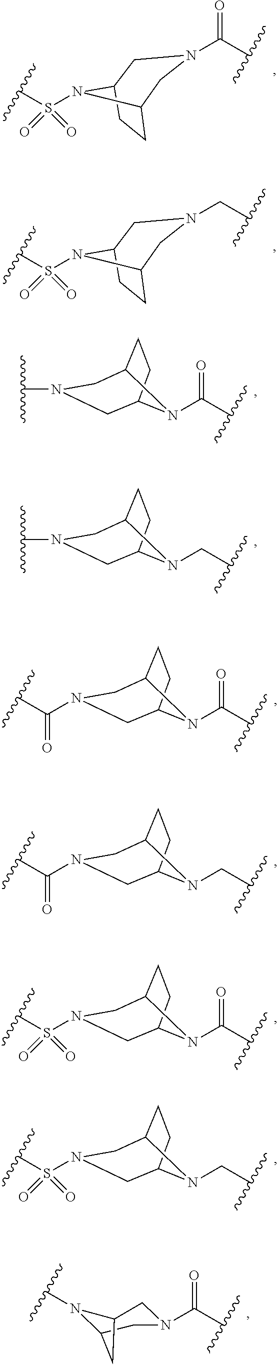 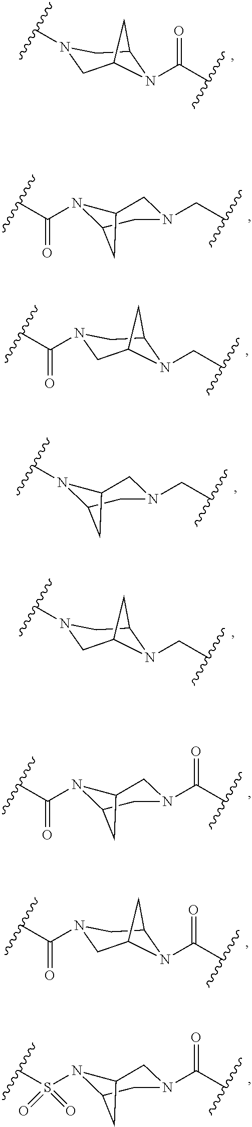 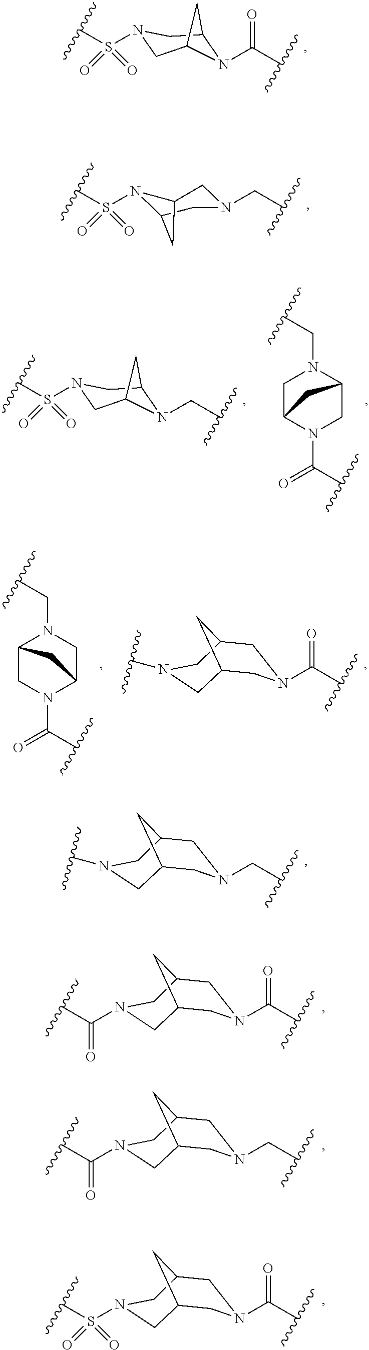 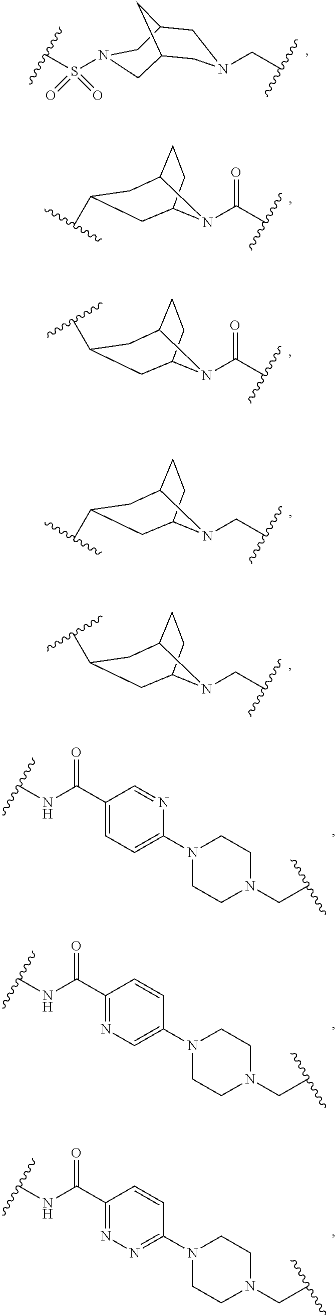 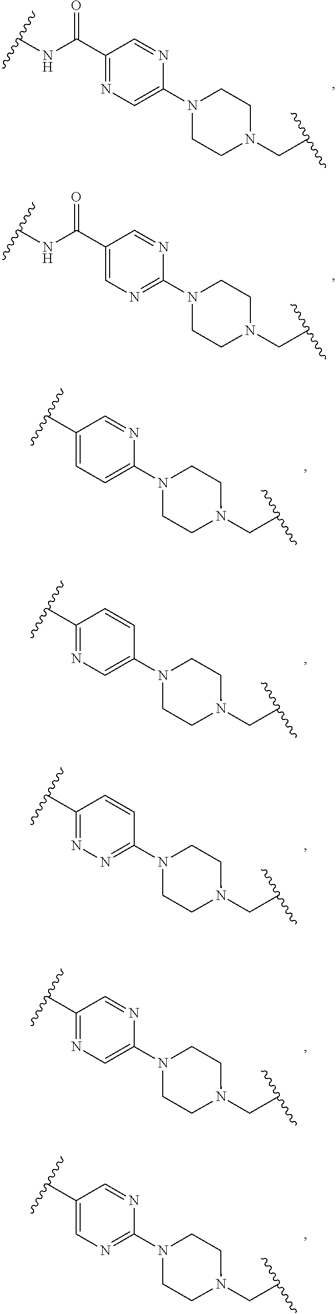 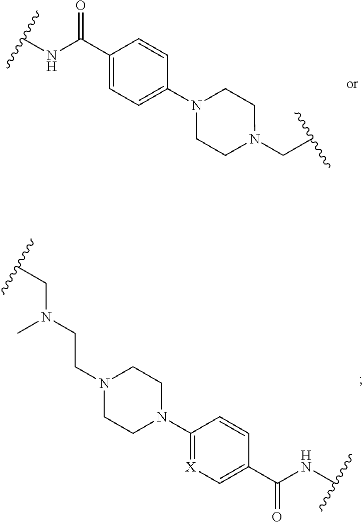
L₂ is a bond, 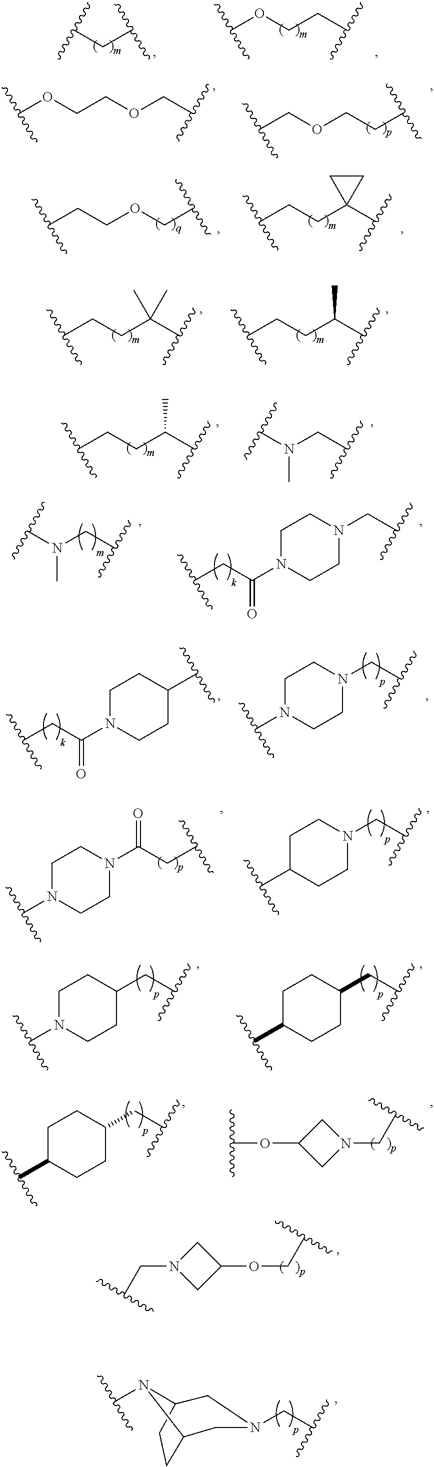 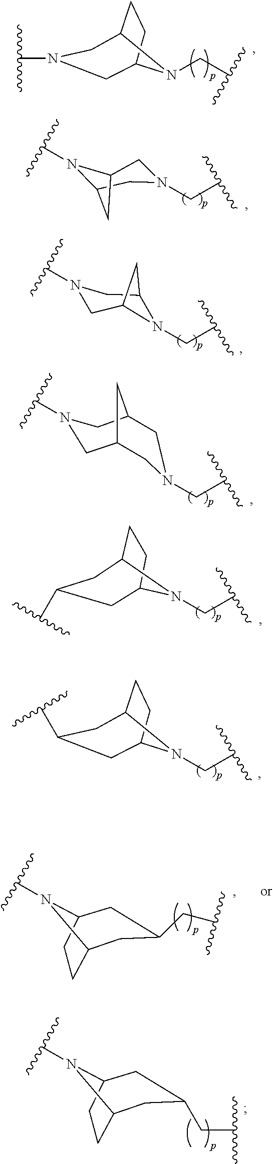
each X is independently CH or N
each k is independently 0, 1, 2, 3, 4, 5, or 6;
each m is independently 2, 3, 4, 5, 6, or 7;
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl.

2. The compound of claim 1, or a pharmaceutically acceptable salt thereof, wherein:
L₁ is 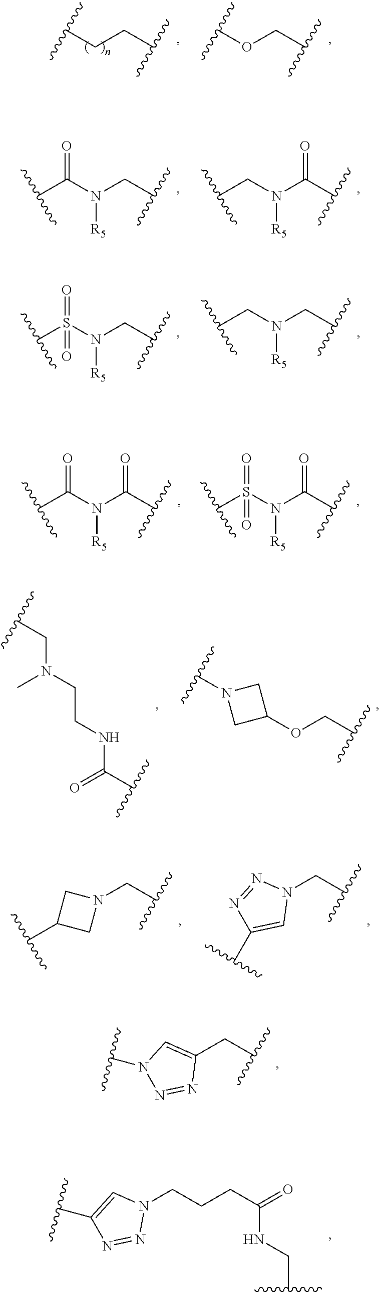 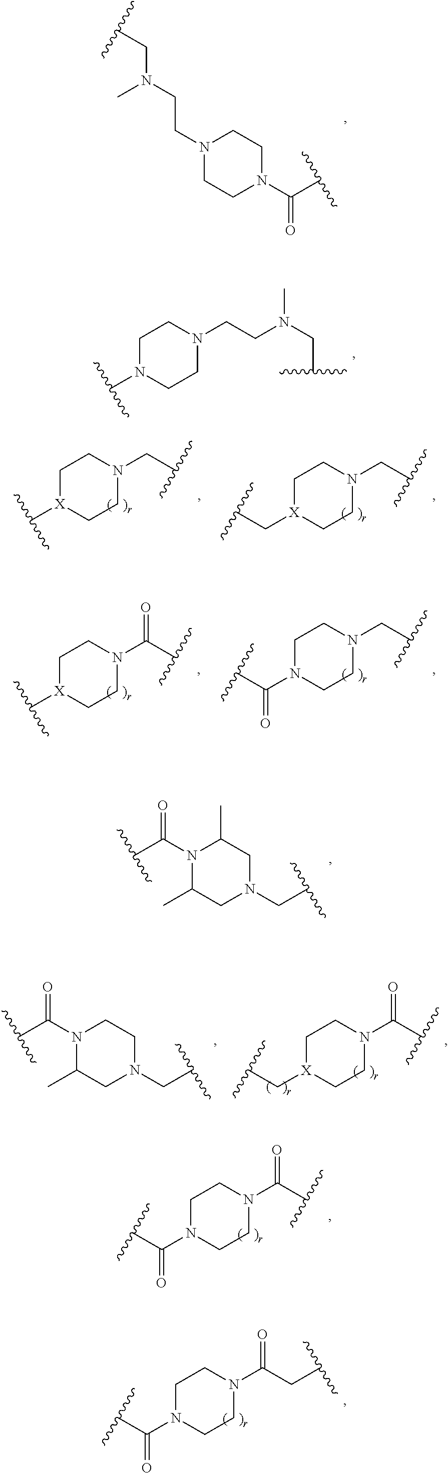 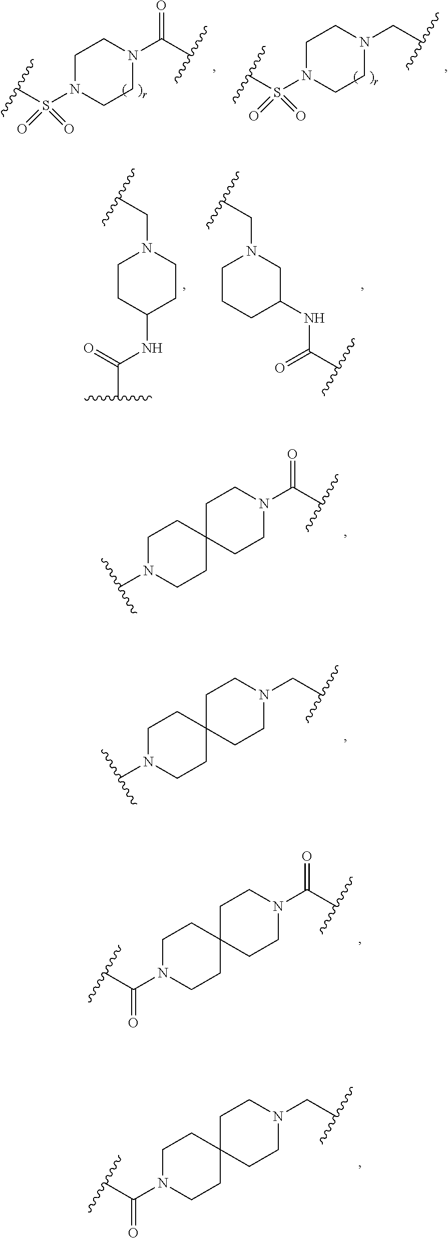 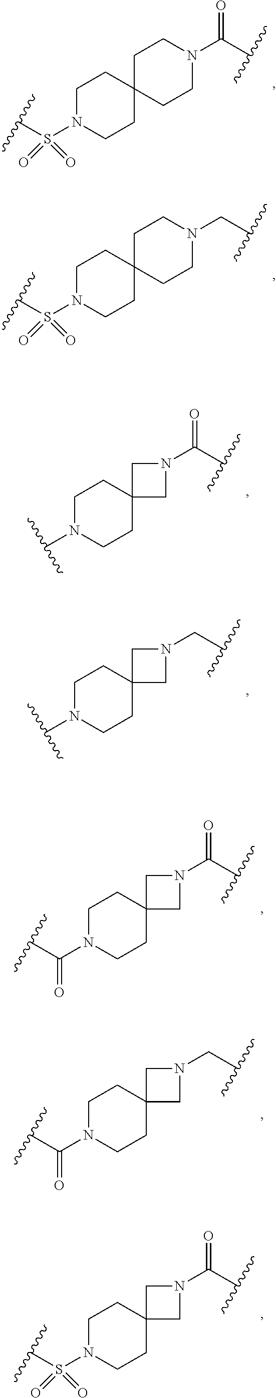 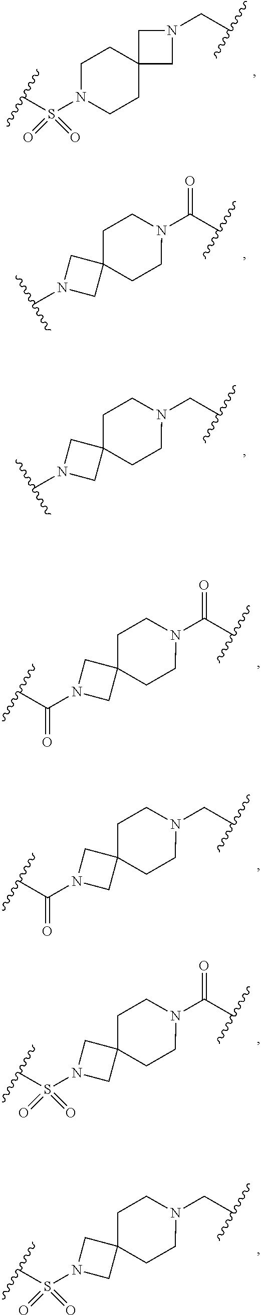 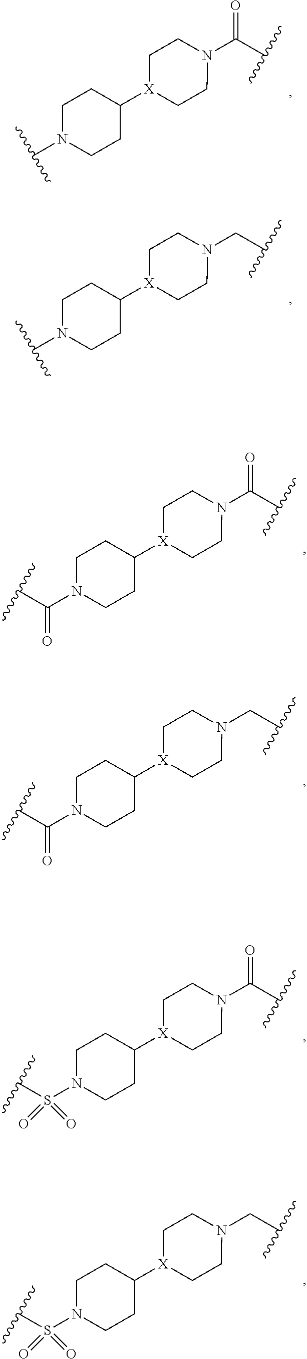 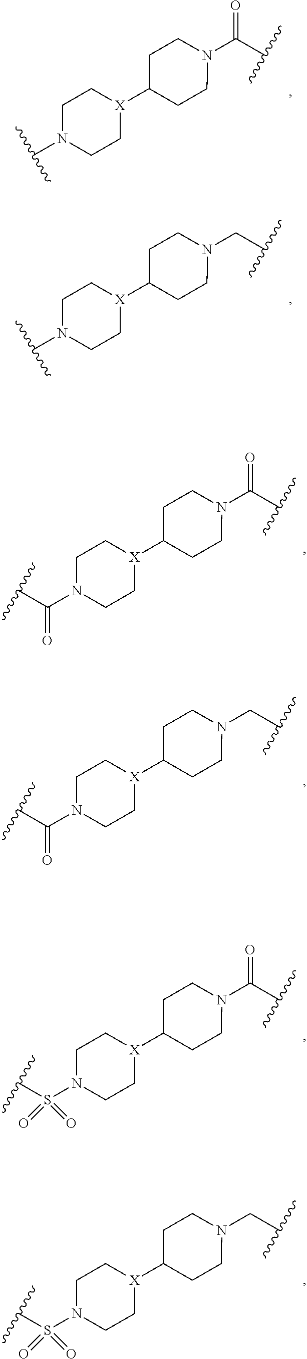 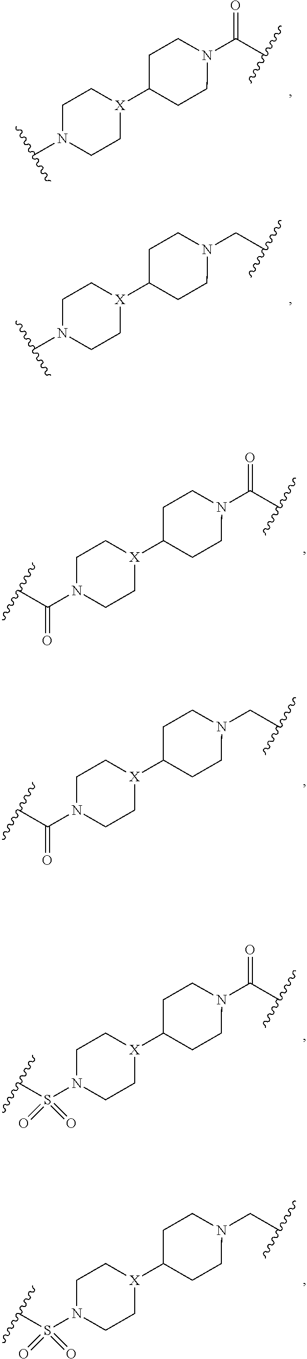 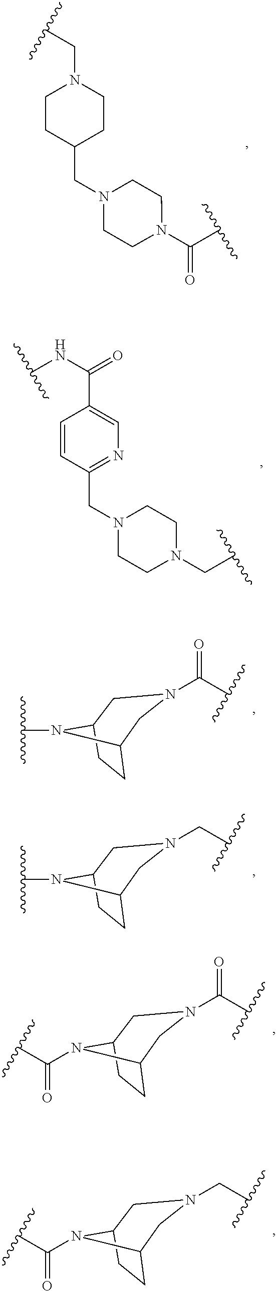 [SMILES: *CN1CCC(CN2CCN(C(*)=O)CC2)CC1 ; *CN1CCN(Cc2ccc(C(=O)N*)cn2)CC1 ; *C(=O)N1CC2CCC(C1)N2* ; *CN1CC2CCC(C1)N2* ; *C(=O)N1CC2CCC(C1)N2C(*)=O ; *CN1CC2CCC(C1)N2C(*)=O] 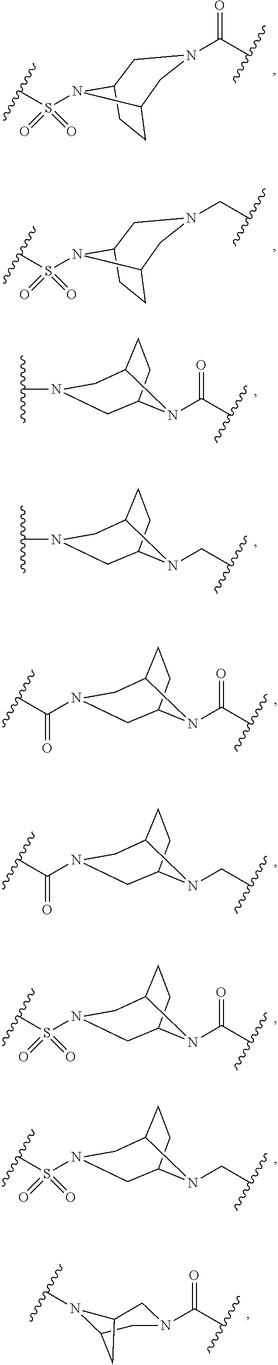 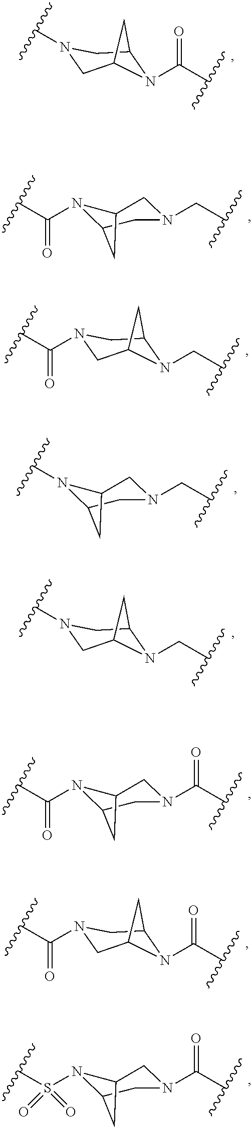 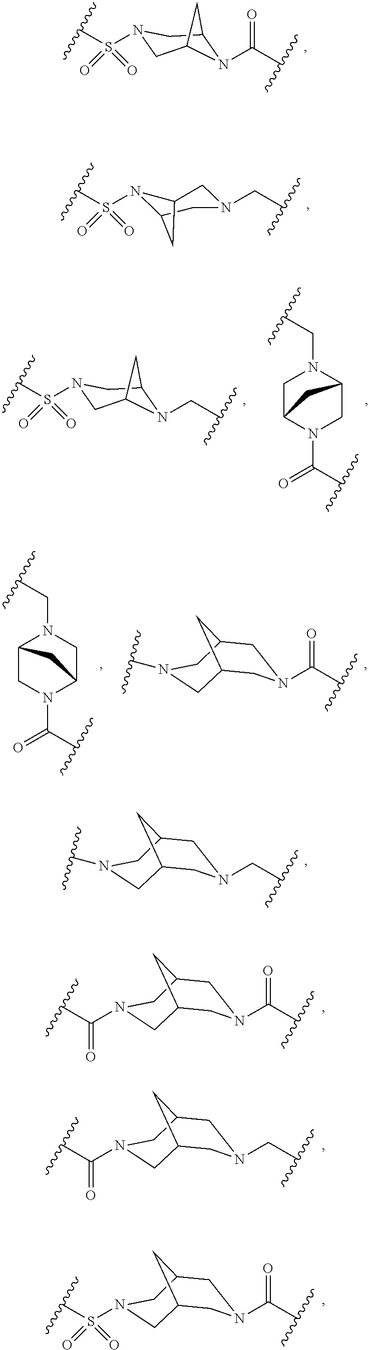 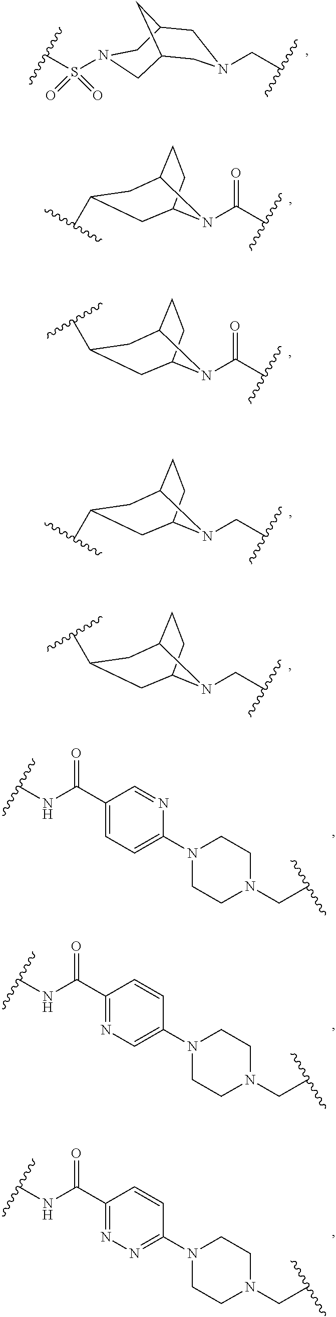 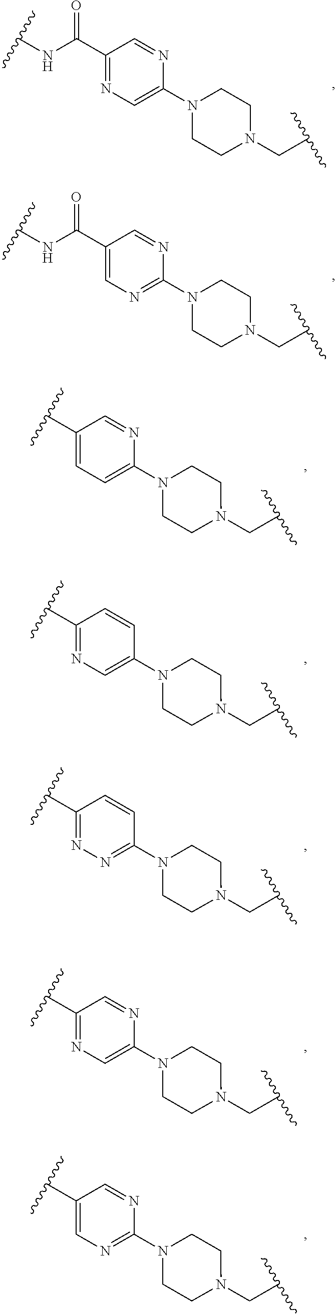 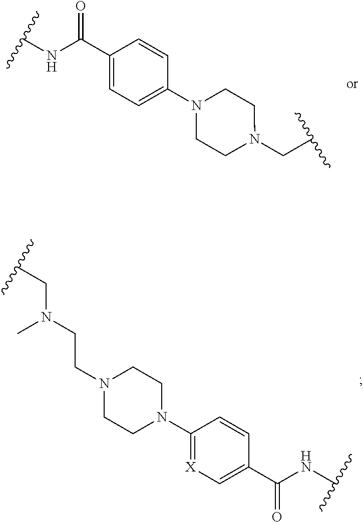
and
L₂ is a bond,

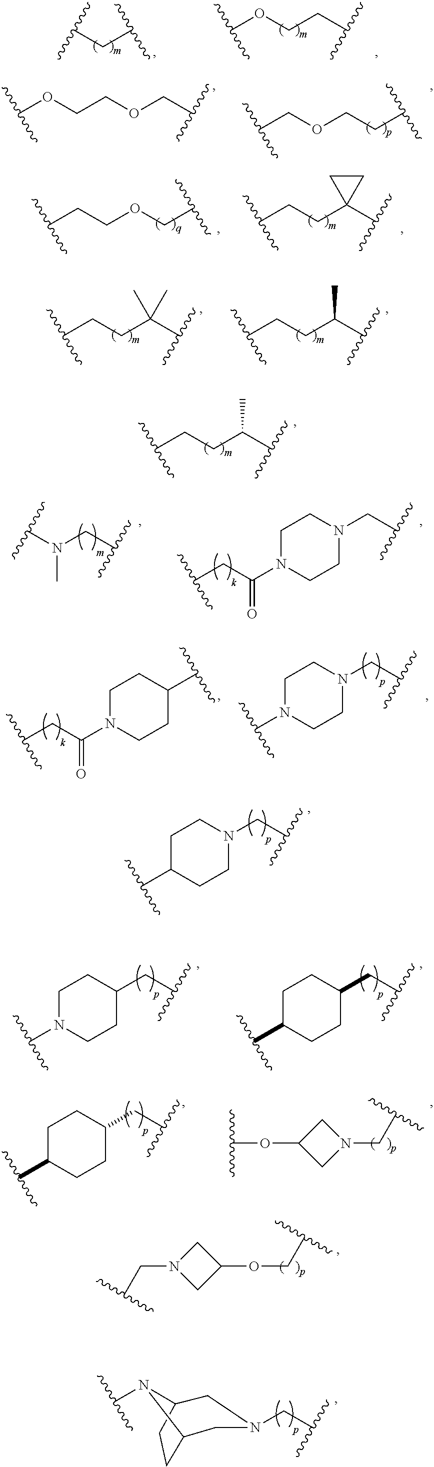

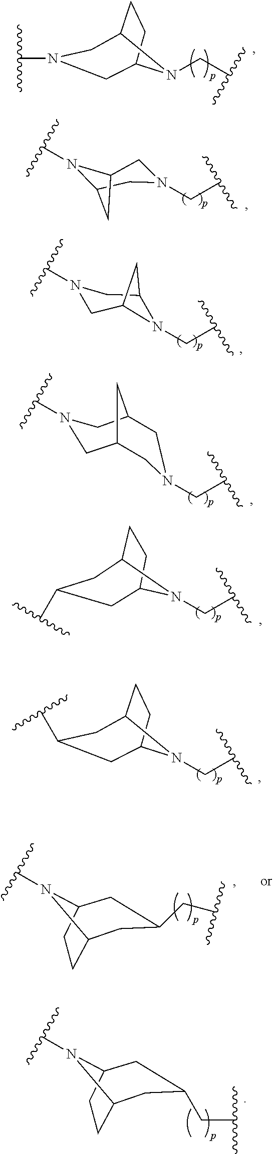

3. The compound of claim 1 or 2, or a pharmaceutically acceptable salt thereof, wherein the compound is of formula:

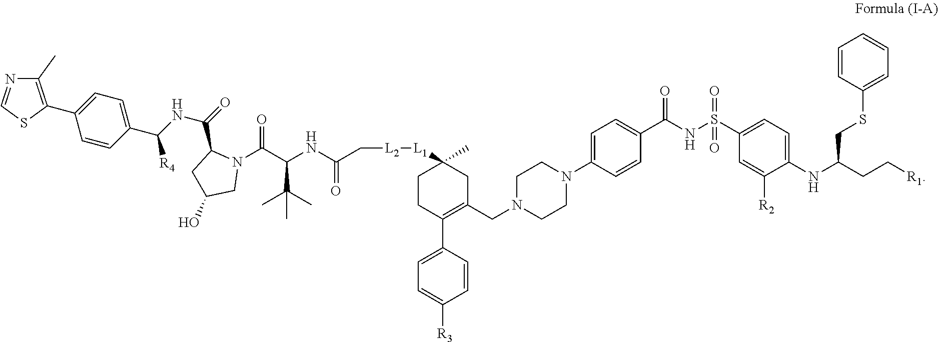

4. The compound of any one of claims 1-3, or a pharmaceutically acceptable salt thereof, wherein L₁ is

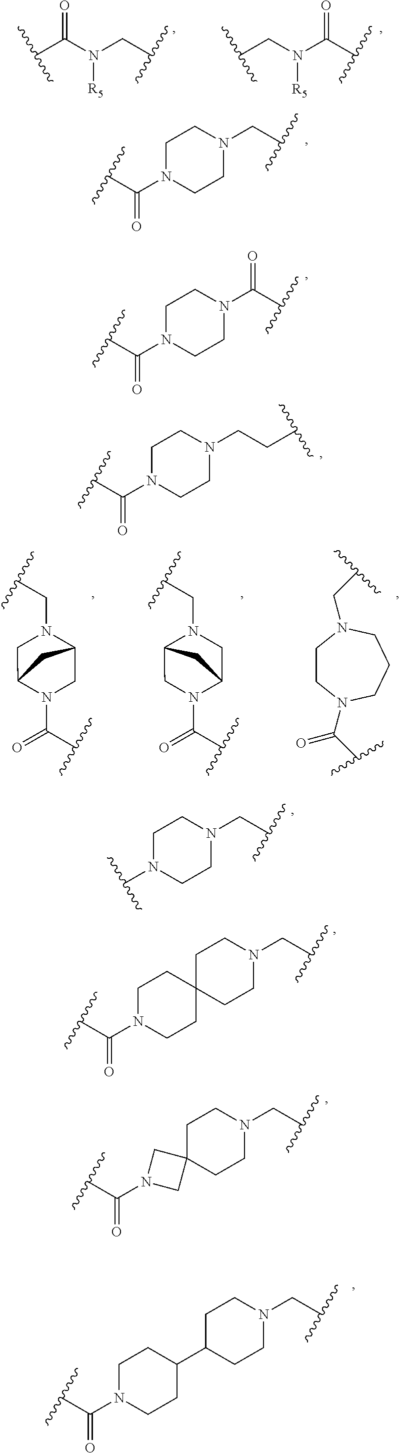

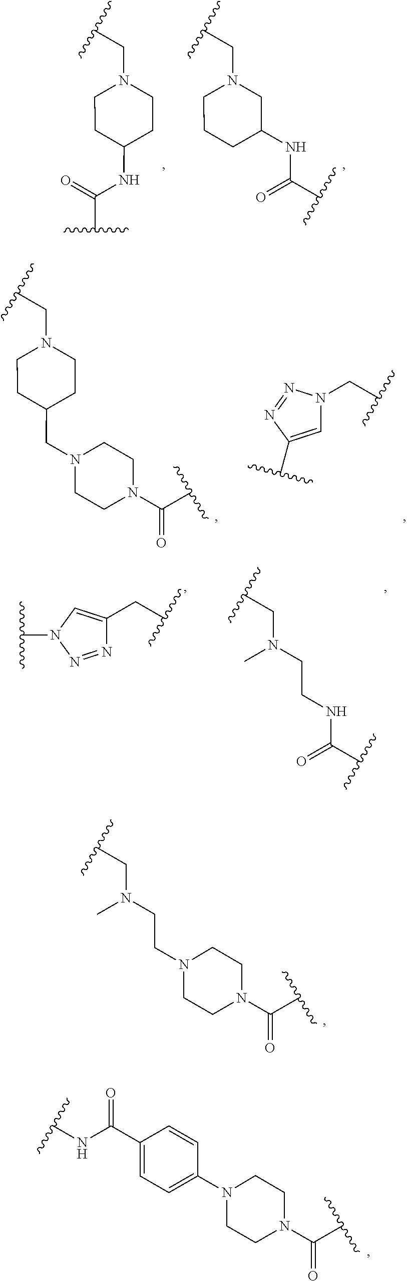

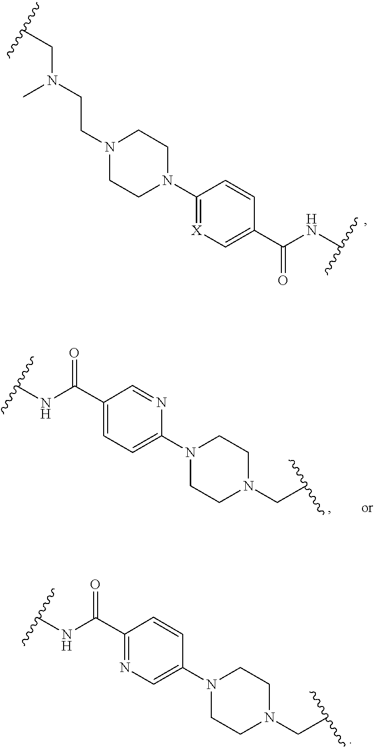

5. The compound of claim 4, or a pharmaceutically acceptable salt thereof, wherein L₁ is

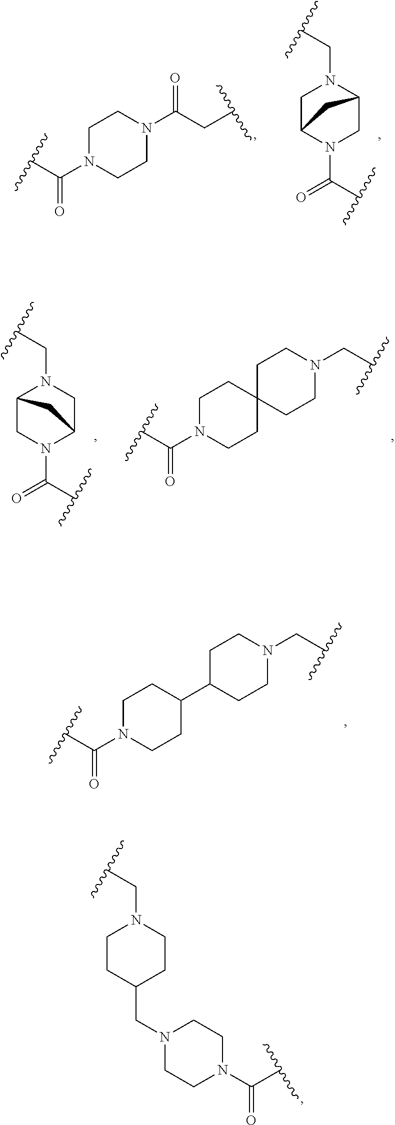 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2 ; *CN1CCC(C2CCN(C(*)=O)CC2)CC1 ; *CN1CCC(CN2CCN(C(*)=O)CC2)CC1]

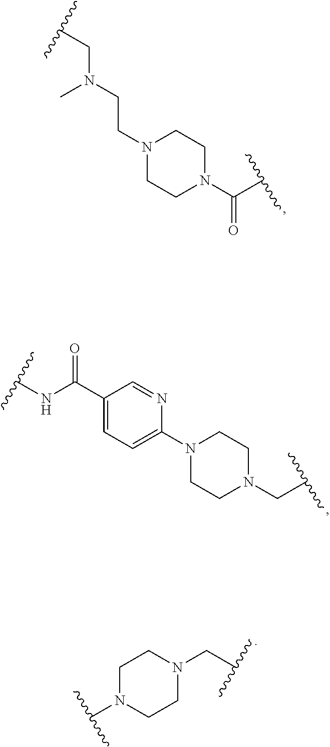 [SMILES: *CN(C)CCN1CCN(C(*)=O)CC1 ; *CN1CCN(c2ccc(C(=O)N*)cn2)CC1 ; *CN1CCN(*)CC1]

6. The compound of any one of claims 1-3, or a pharmaceutically acceptable salt thereof, wherein L₁ is

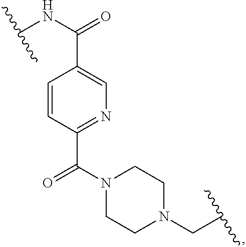 [SMILES: *CN1CCN(C(=O)c2ccc(C(=O)N*)cn2)CC1]

7. The compound of any one of claims 1-6, or a pharmaceutically acceptable salt thereof, wherein L₂ is

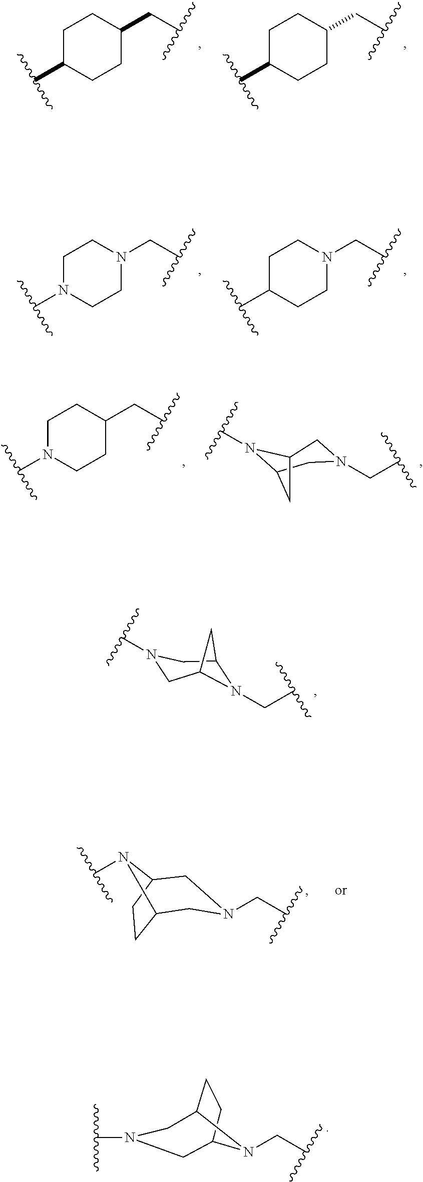

8. The compound of claim 7, or a pharmaceutically acceptable salt thereof, wherein L₂ is

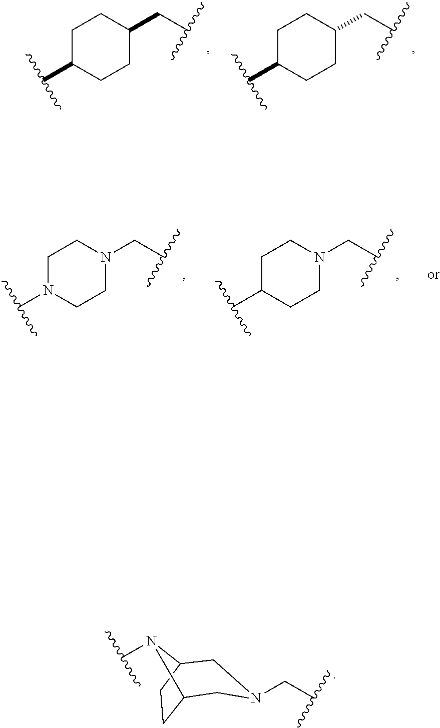 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

9. The compound of any one of claims 1-6, or a pharmaceutically acceptable salt thereof, wherein L₂ is

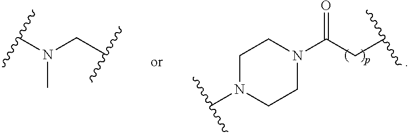

10. The compound of any one of claims 1-9, or a pharmaceutically acceptable salt thereof, wherein R₁ is

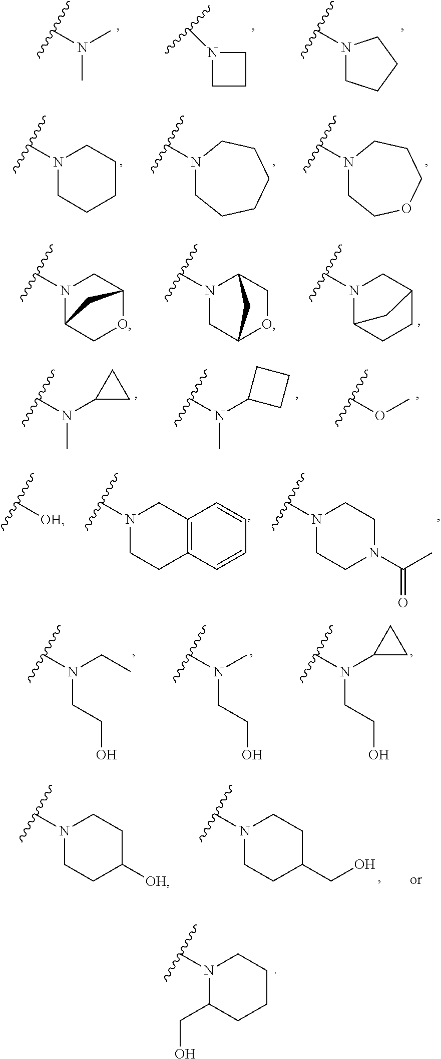

11. The compound of claim 10, or a pharmaceutically acceptable salt thereof, wherein R₁ is

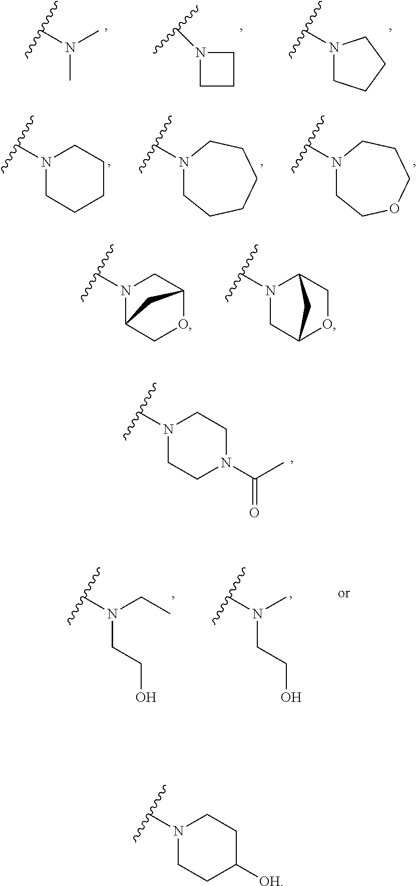

12. The compound of claim 1, 10, or 11, or a pharmaceutically acceptable salt thereof, wherein L₁ is 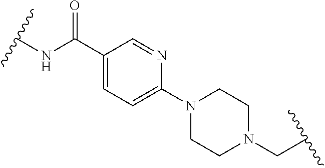 [SMILES: *CN1CCN(c2ccc(C(=O)N*)cn2)CC1]
and L₂ is a bond or

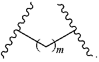

13. The compound of claim 1, 10, or 11, or a pharmaceutically acceptable salt thereof, wherein L₁ is 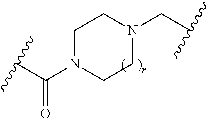
and L₂ is

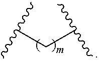

14. The compound of claim 1, 10, or 11, or a pharmaceutically acceptable salt thereof, wherein L₁ is 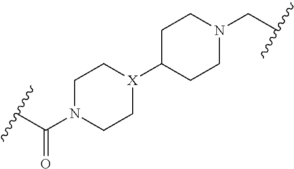
and L₂ is

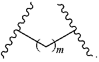

15. The compound of claim 1, 10, or 11, or a pharmaceutically acceptable salt thereof, wherein L₁ is 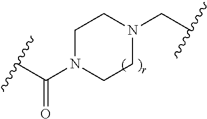
and L₂ is

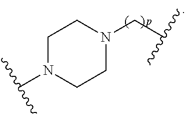

16. The compound of claim 1, 10, or 11, or a pharmaceutically acceptable salt thereof, wherein:
L₂ is 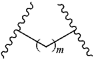
(e.g., wherein m is 1), and L₁ is 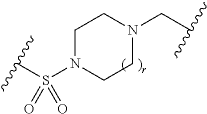
(e.g., wherein r is 1);
L₂ is 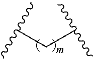
(e.g., wherein m is 3), and L₁ is 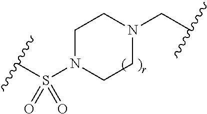
(e.g., wherein r is 1);
L₂ is 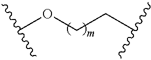
(e.g., wherein m is 2), and L₁ is 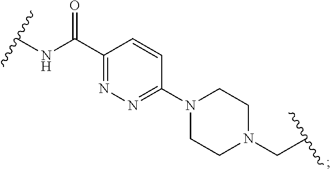 [SMILES: *CN1CCN(c2ccc(C(=O)N*)nn2)CC1]
L₂ is 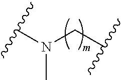
(e.g., wherein m is 3), and L₁ is 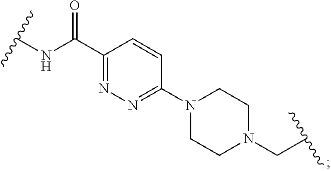 [SMILES: *CN1CCN(c2ccc(C(=O)N*)nn2)CC1]
L₂ is 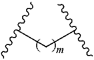
(e.g., wherein m is 4), and L₁ is 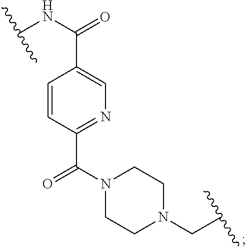 [SMILES: *CN1CCN(C(=O)c2ccc(C(=O)N*)cn2)CC1]
L₂ is 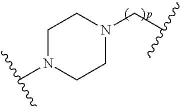
(e.g., wherein p is 2), and L₁ is 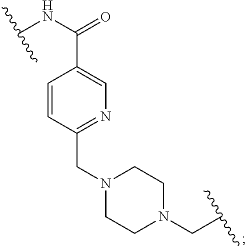 [SMILES: *CN1CCN(Cc2ccc(C(=O)N*)cn2)CC1]
L₂ is 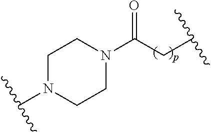
(e.g., wherein p is 1), and L₁ is 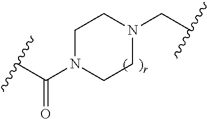
(e.g., wherein r is 1);
L₂ is 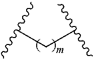
(e.g., wherein m is 3), and L₁ is 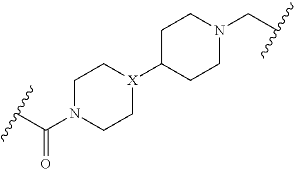
(e.g., wherein X is CH);
L₂ is 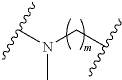
(e.g., wherein m is 2), and L₁ is 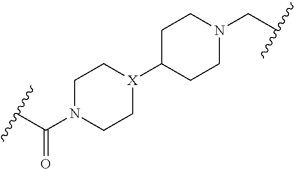
(e.g., wherein X is CH);
L₂ is 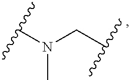 [SMILES: *CN(*)C]
and L₁ is 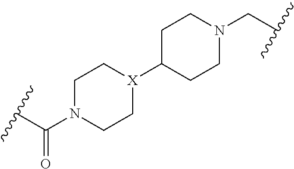
(e.g., wherein X is CH);
L₂ is 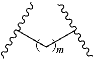
(e.g., wherein m is 3), and L₁ is 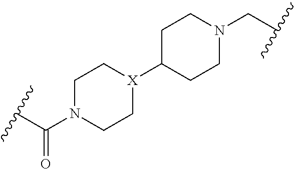
(e.g., wherein X is N); or
L₂ is 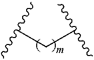
(e.g., wherein m is 3), and L₁ is 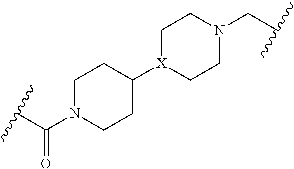
(e.g., wherein X is N).

17. The compound of any one of claims 1-16, or a pharmaceutically acceptable salt thereof, wherein R₂ is NO₂.

18. The compound of any one of claims 1-16, or a pharmaceutically acceptable salt thereof, wherein R₂ is SO₂CF₃.

19. The compound of any one of claims 1-18, or a pharmaceutically acceptable salt thereof, wherein R₃ is F, Cl, or CF₃.

20. The compound of claim 19, or a pharmaceutically acceptable salt thereof, wherein R₃ is Cl.

21. The compound of any one of claims 1-20, or a pharmaceutically acceptable salt thereof, wherein R₄ is CH₃.

22. The compound of any one of claims 1-20, or a pharmaceutically acceptable salt thereof, wherein R₄ is H.

23. The compound of any one of claims 1-22, or a pharmaceutically acceptable salt thereof, wherein R₅ is H.

24. The compound of any one of claims 1-22, or a pharmaceutically acceptable salt thereof, wherein R₅ is optionally substituted C₁₋₆ alkyl.

25. The compound of claim 24, or a pharmaceutically acceptable salt thereof, wherein R₅ is CH₃.

26. The compound of any one of claims 1-25, or a pharmaceutically acceptable salt thereof, wherein X is N.

27. The compound of any one of claims 1-25, or a pharmaceutically acceptable salt thereof, wherein X is CH.

28. The compound of any one of claims 1-27, or a pharmaceutically acceptable salt thereof, wherein r is 1.

29. The compound of any one of claims 1-27, or a pharmaceutically acceptable salt thereof, wherein r is 2.

30. The compound of claim 1, or a pharmaceutically acceptable salt thereof, wherein the compound is: 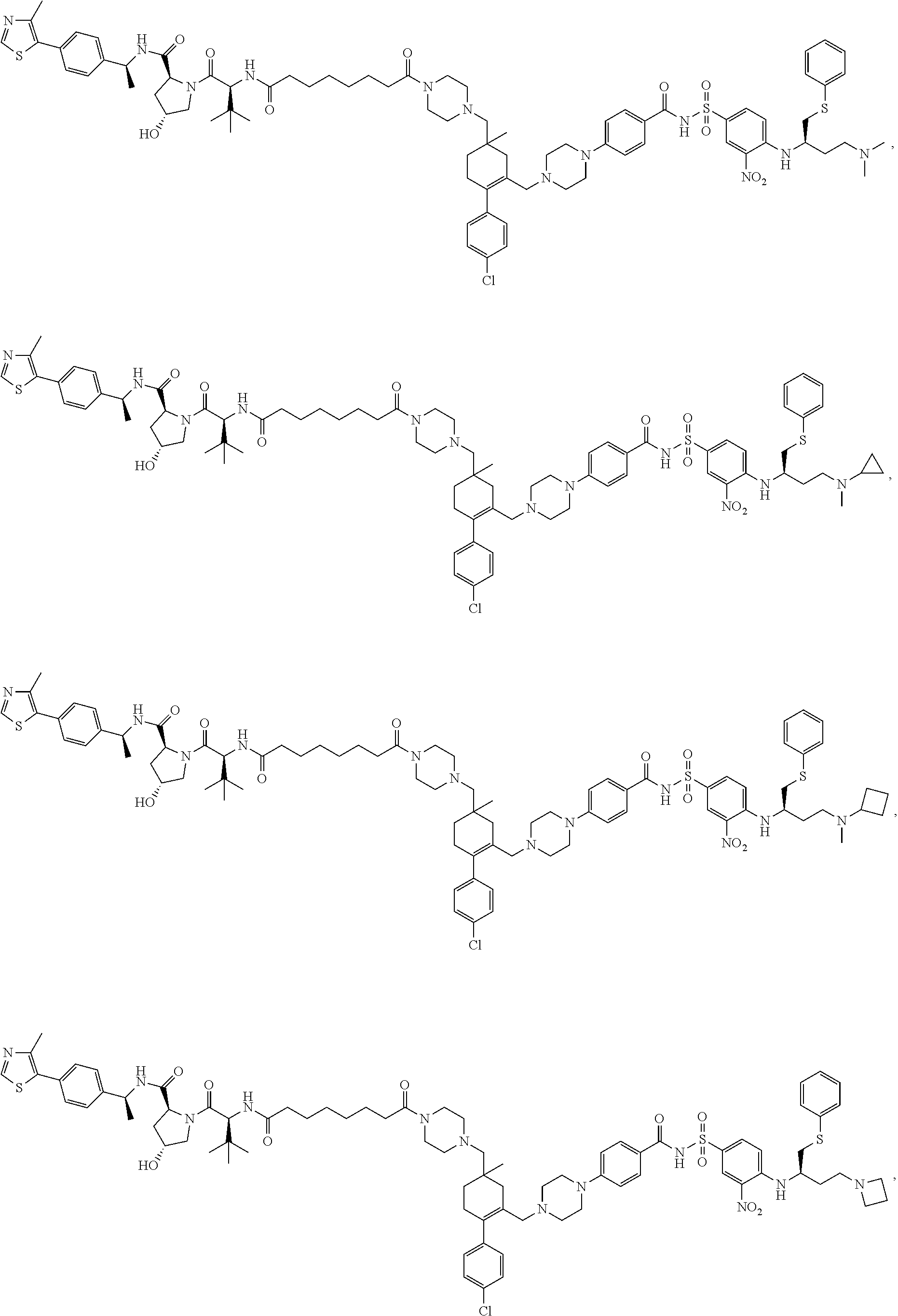 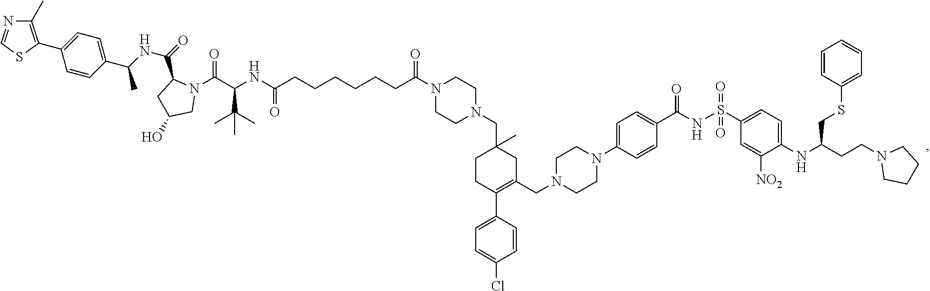 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCCC7)CSc7ccccc7)c([N+](=O)[O-])c6)cc5)CC4)C3)CC2)C(C)(C)C)cc1] 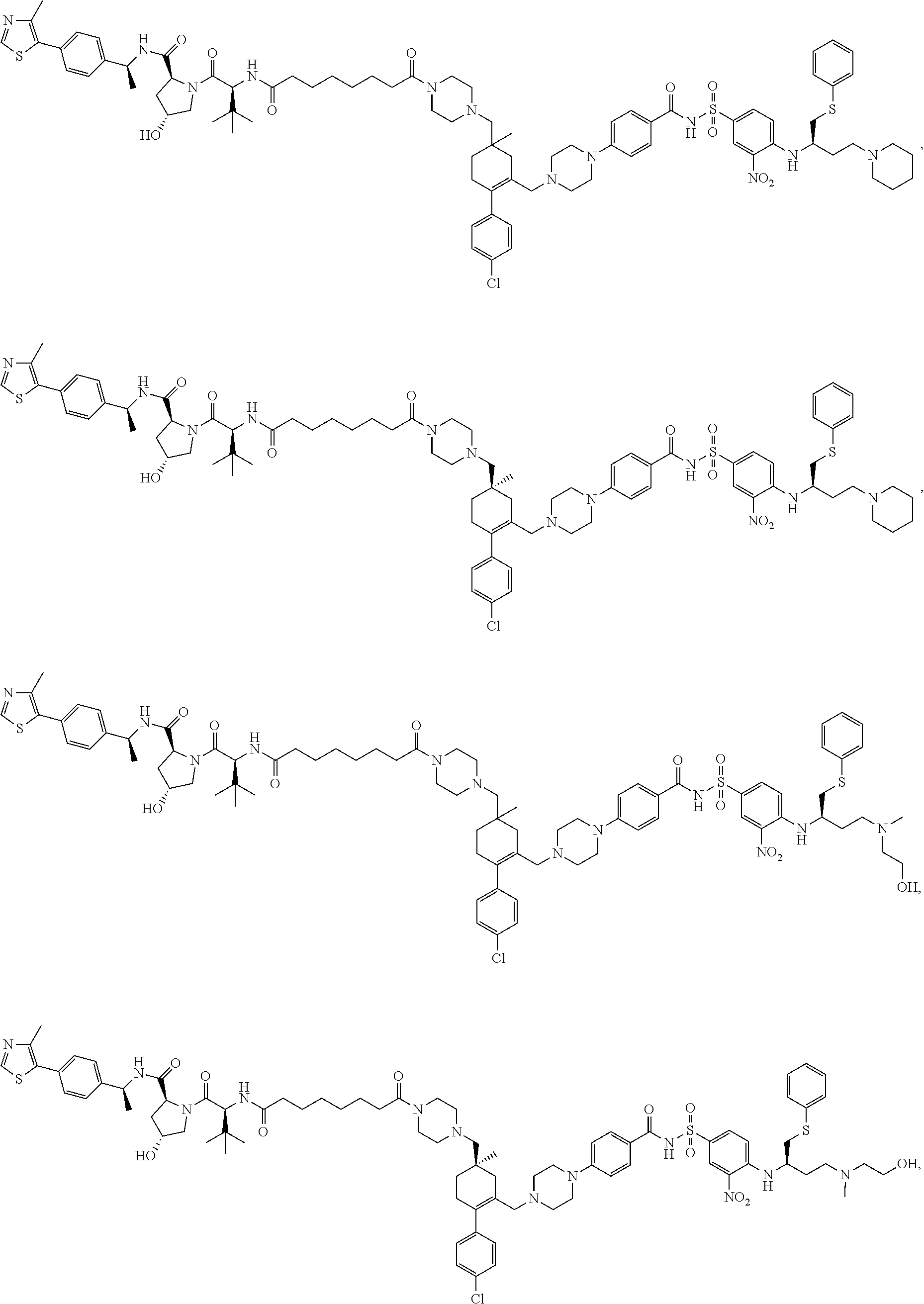 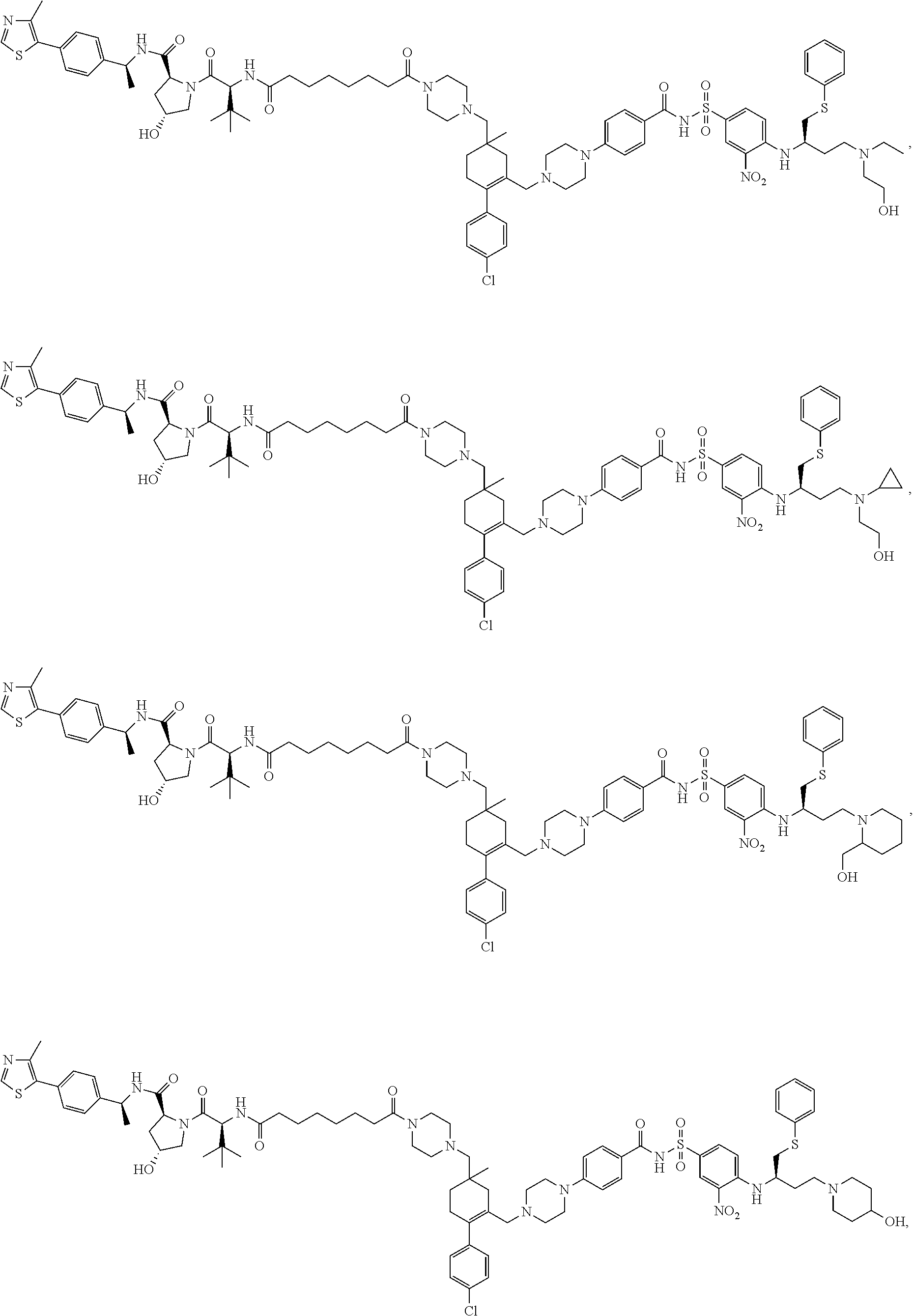 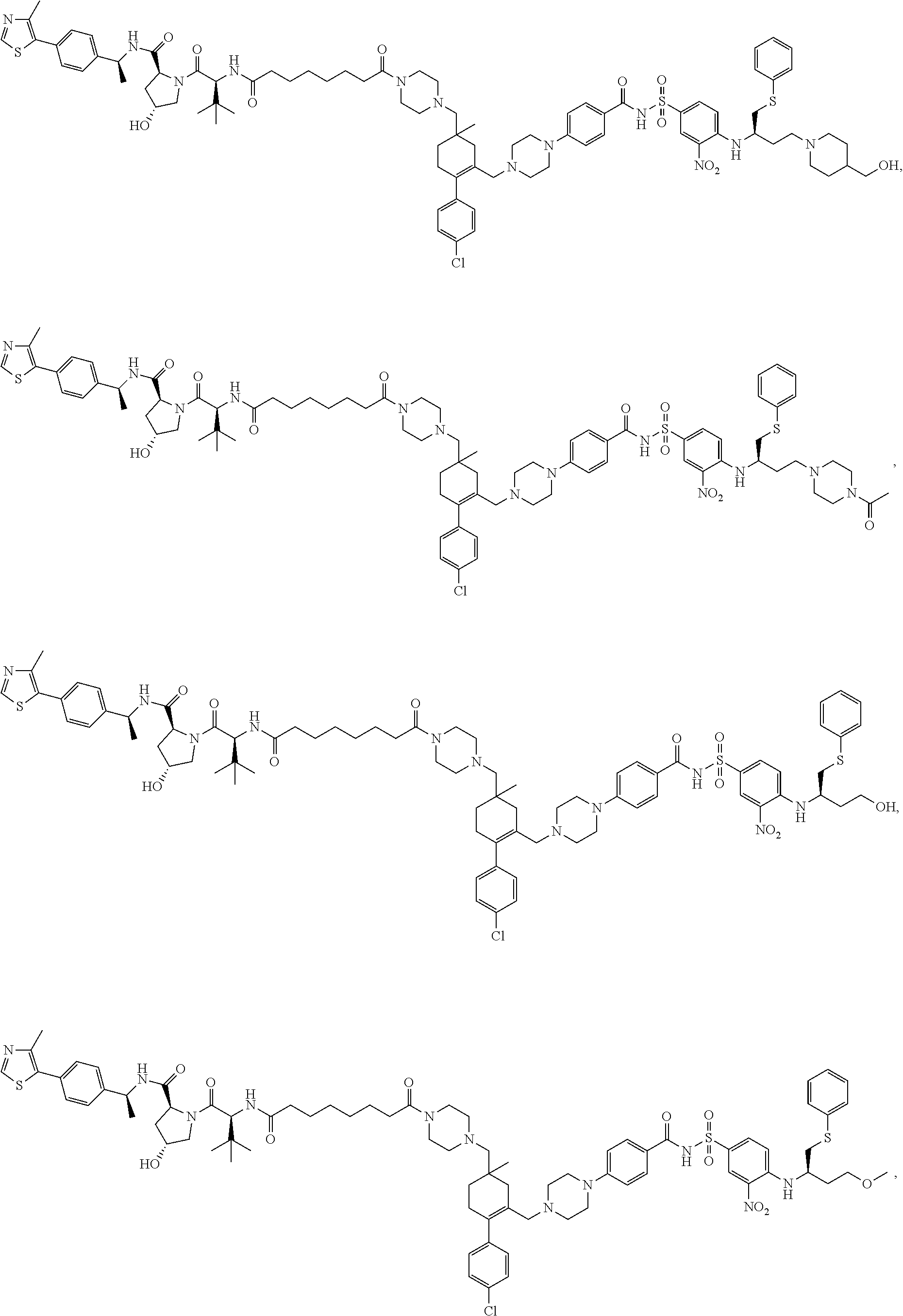 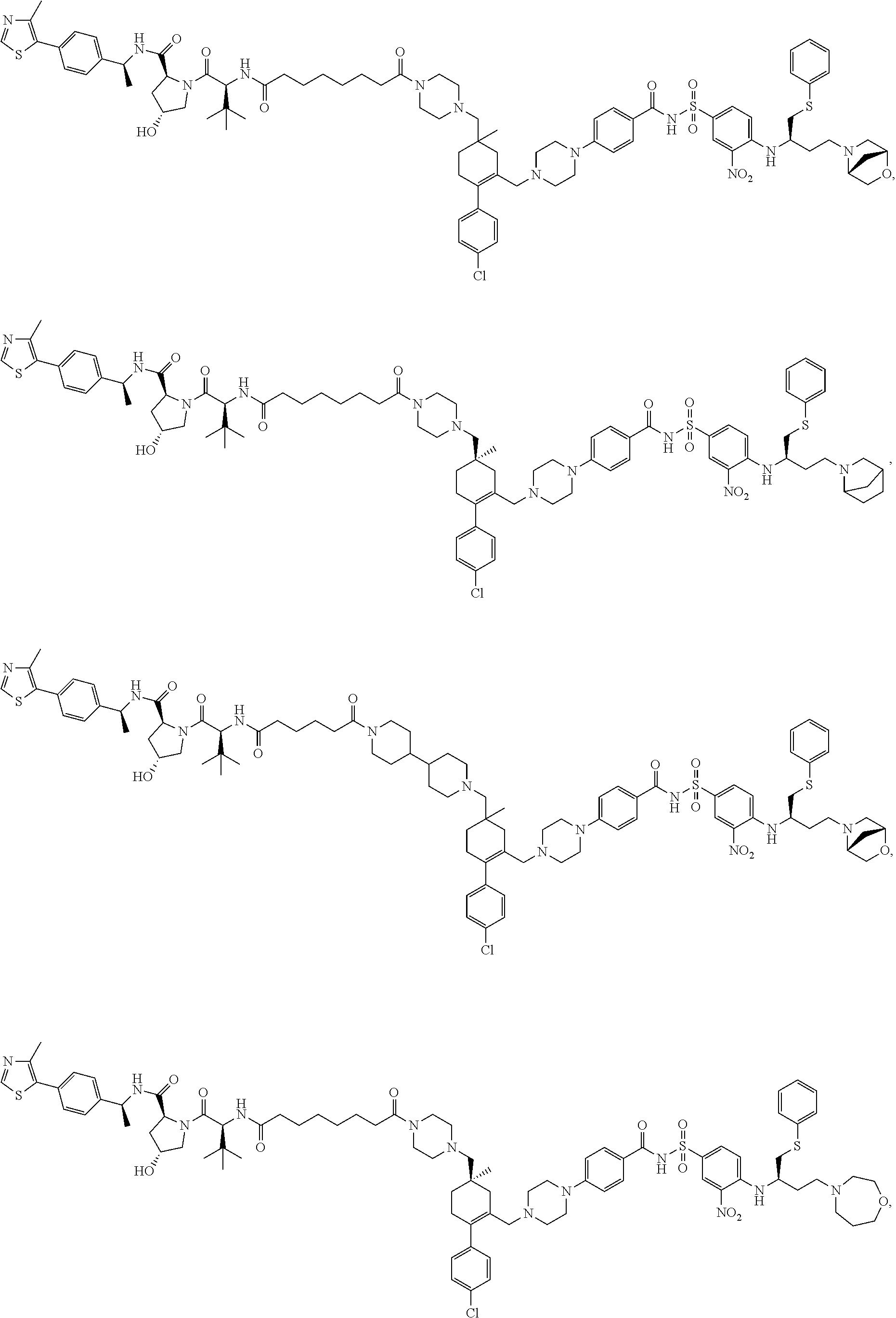 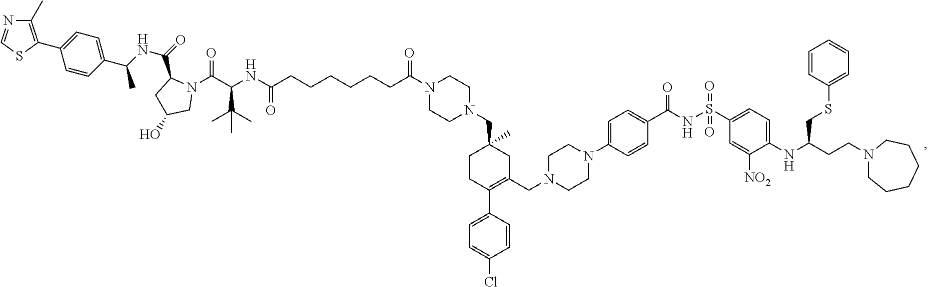 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCCCCC7)CSc7ccccc7)c([N+](=O)[O-])c6)cc5)CC4)C3)CC2)C(C)(C)C)cc1] 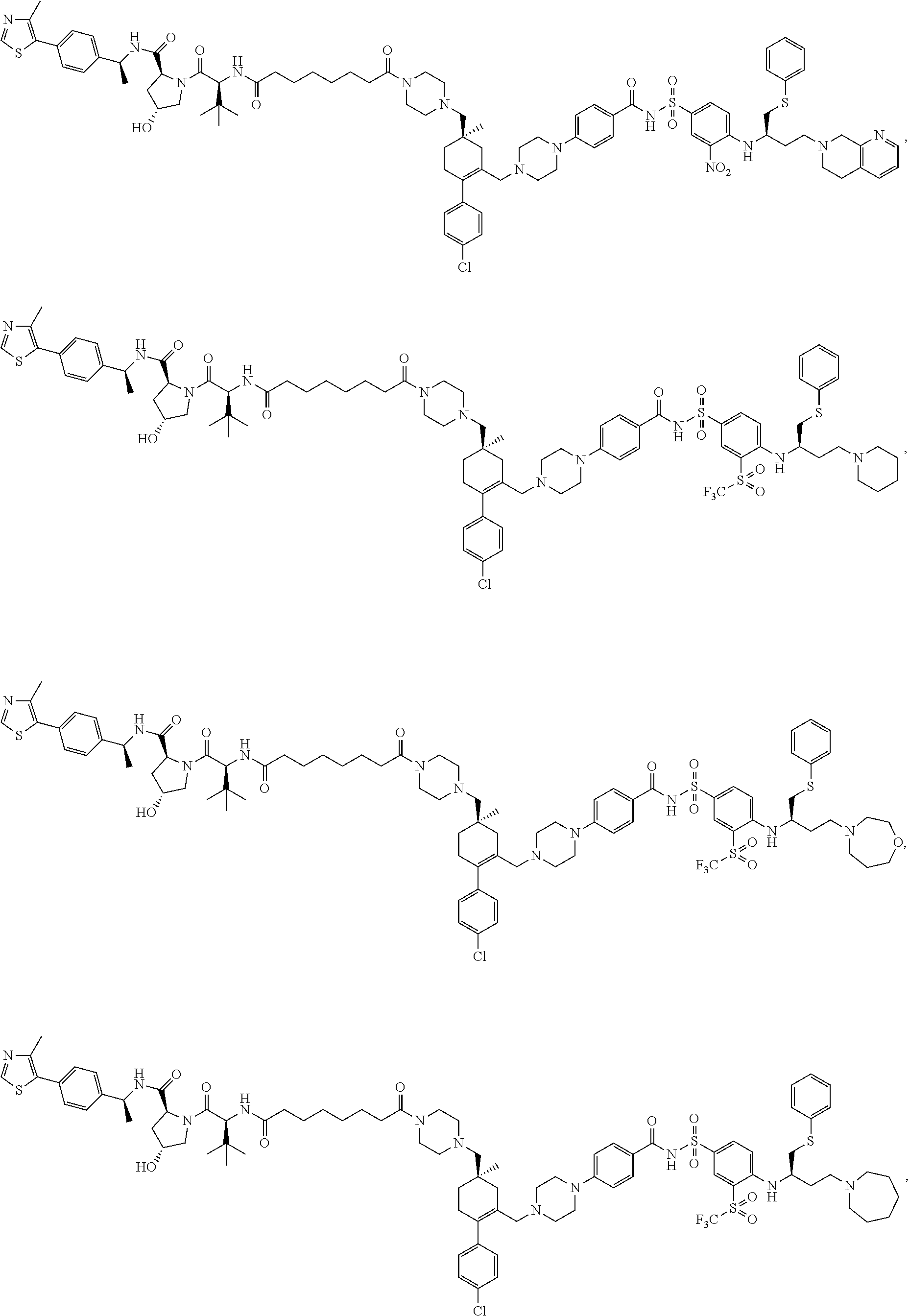 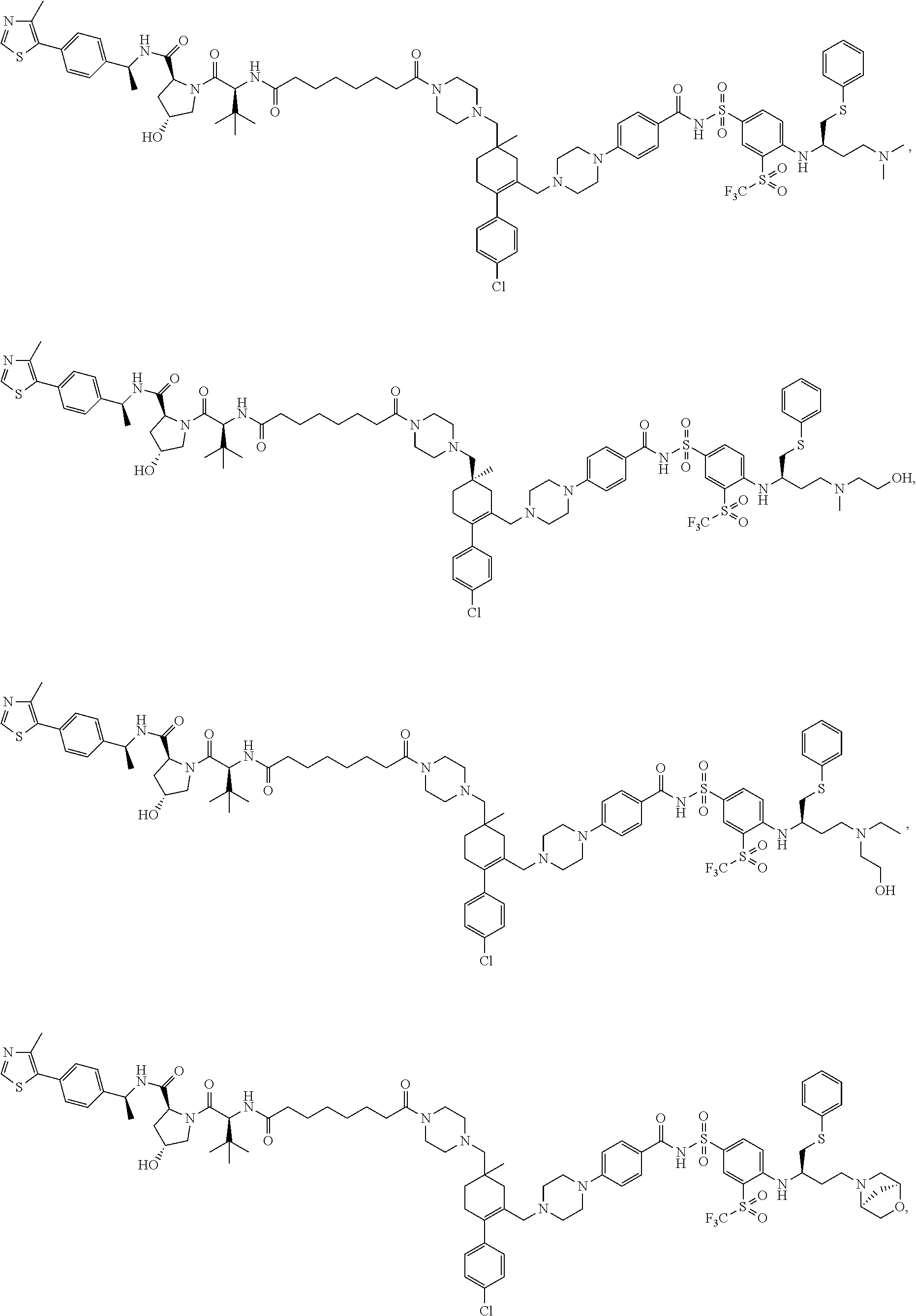 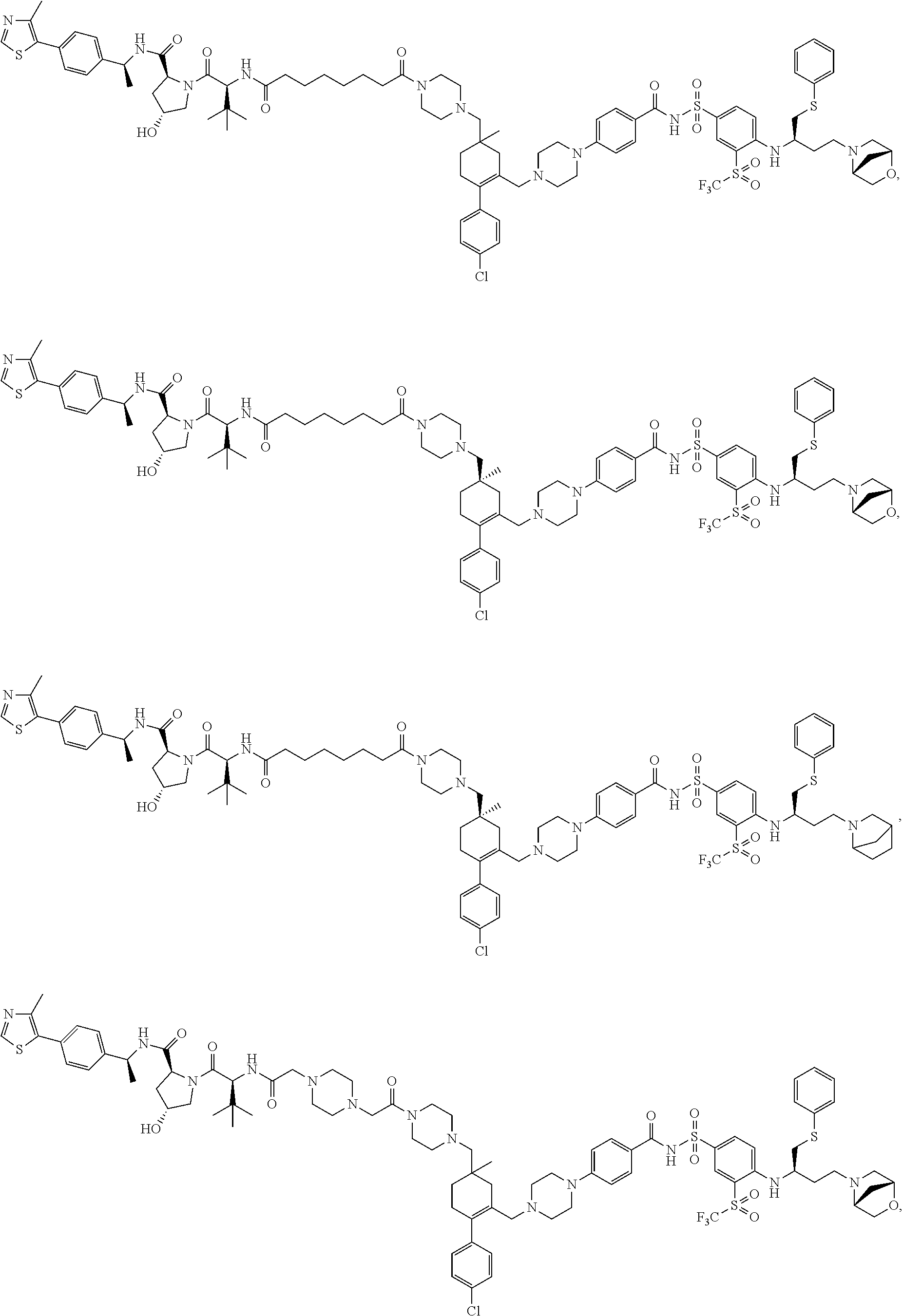 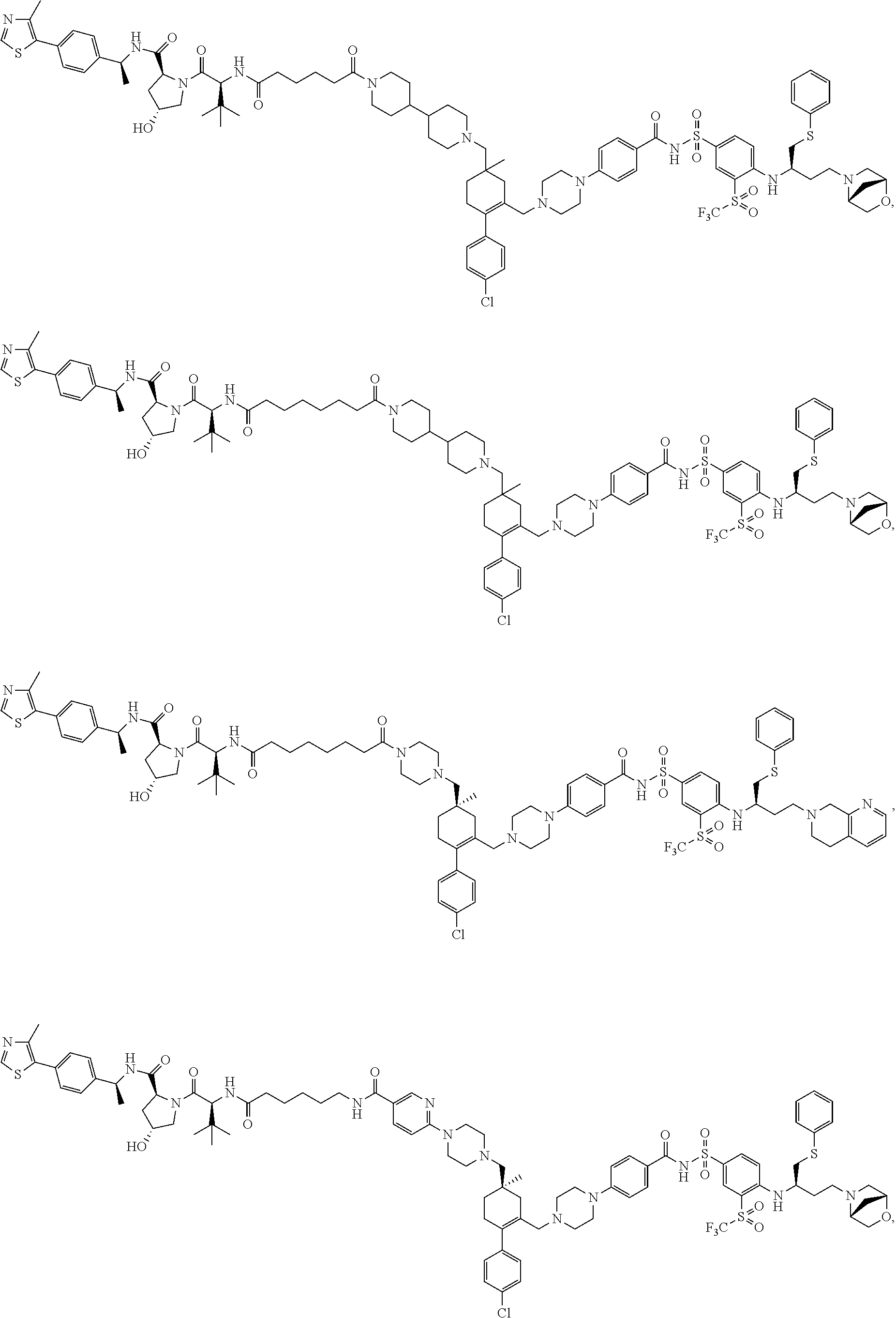 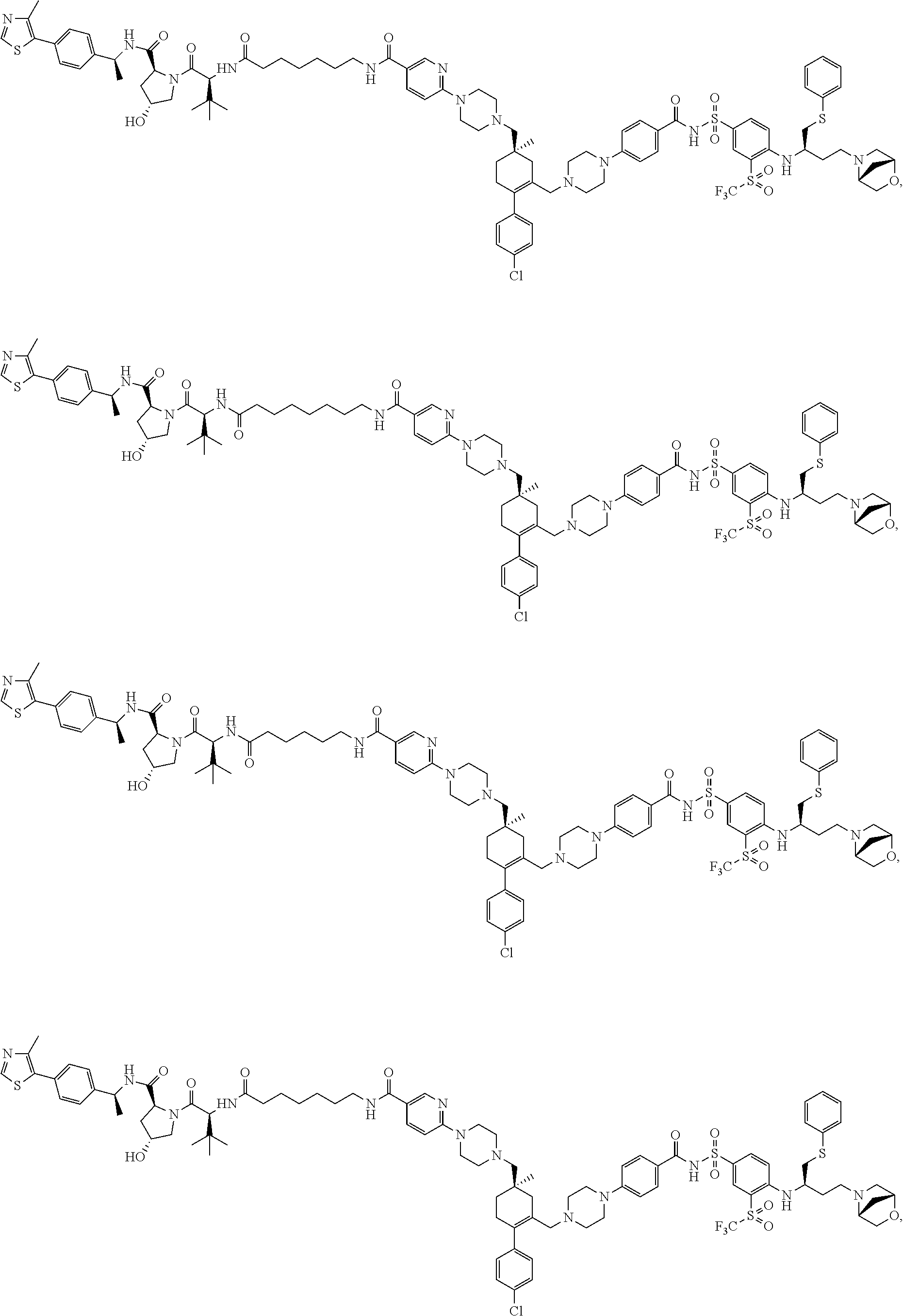 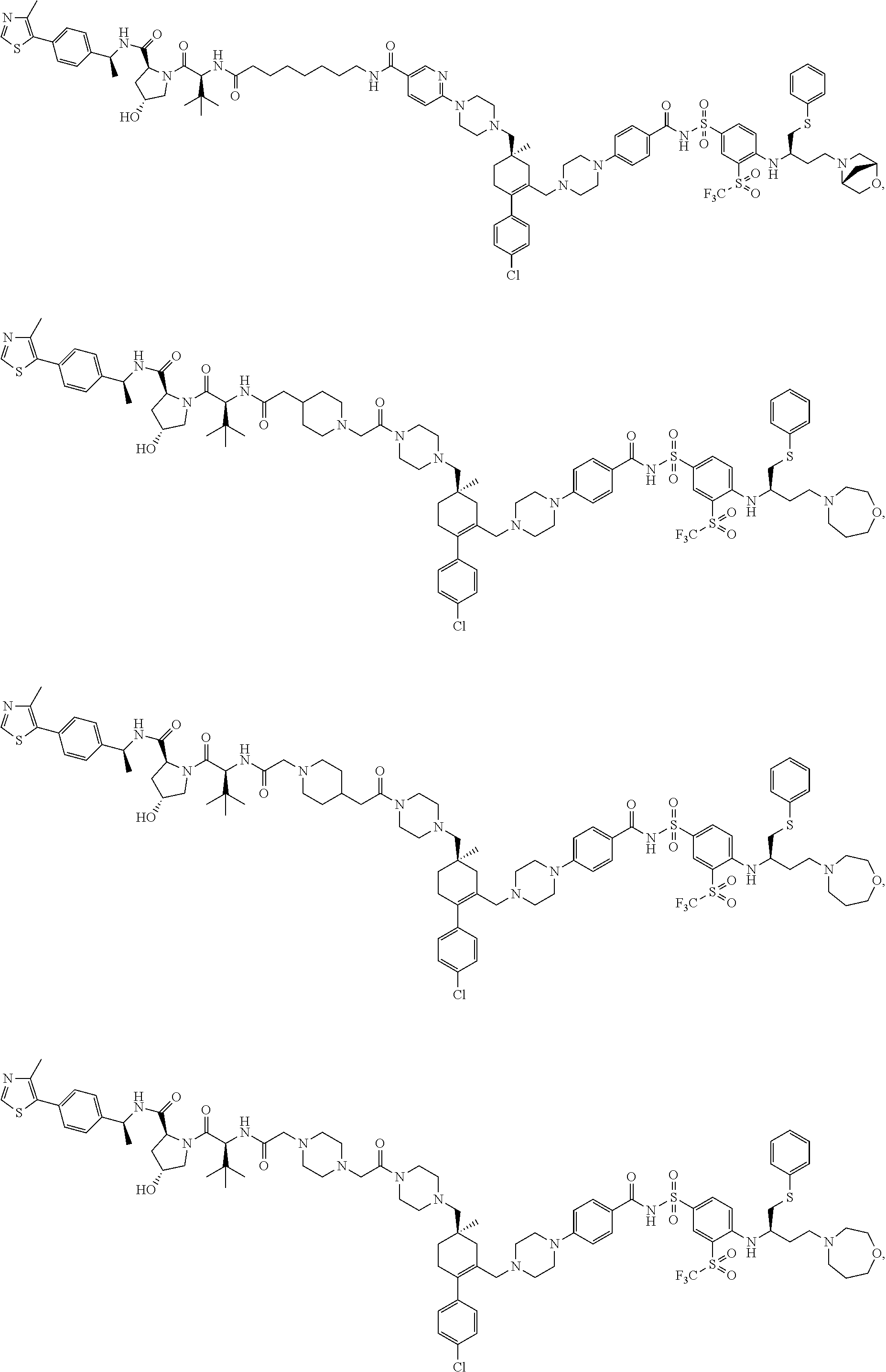 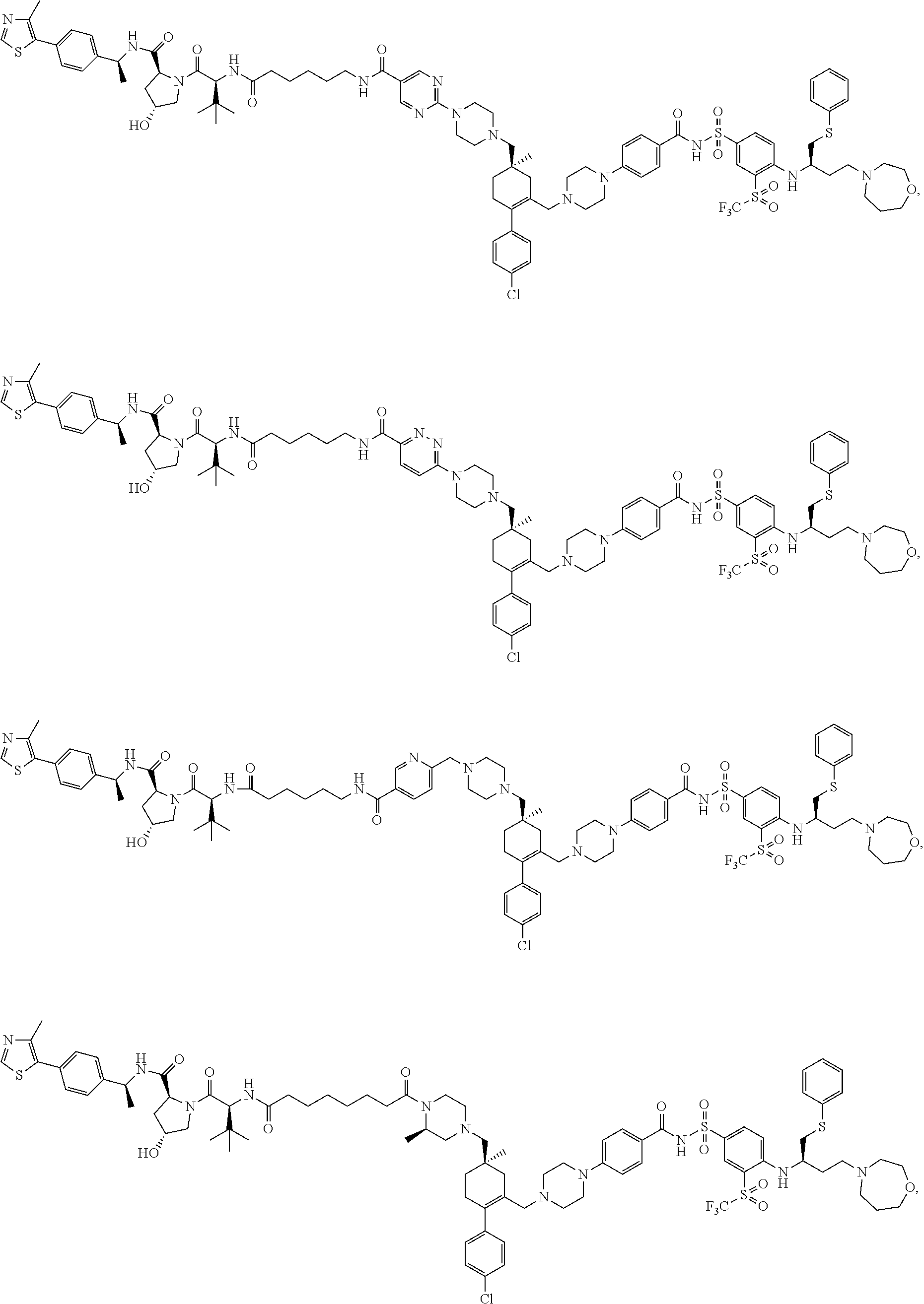 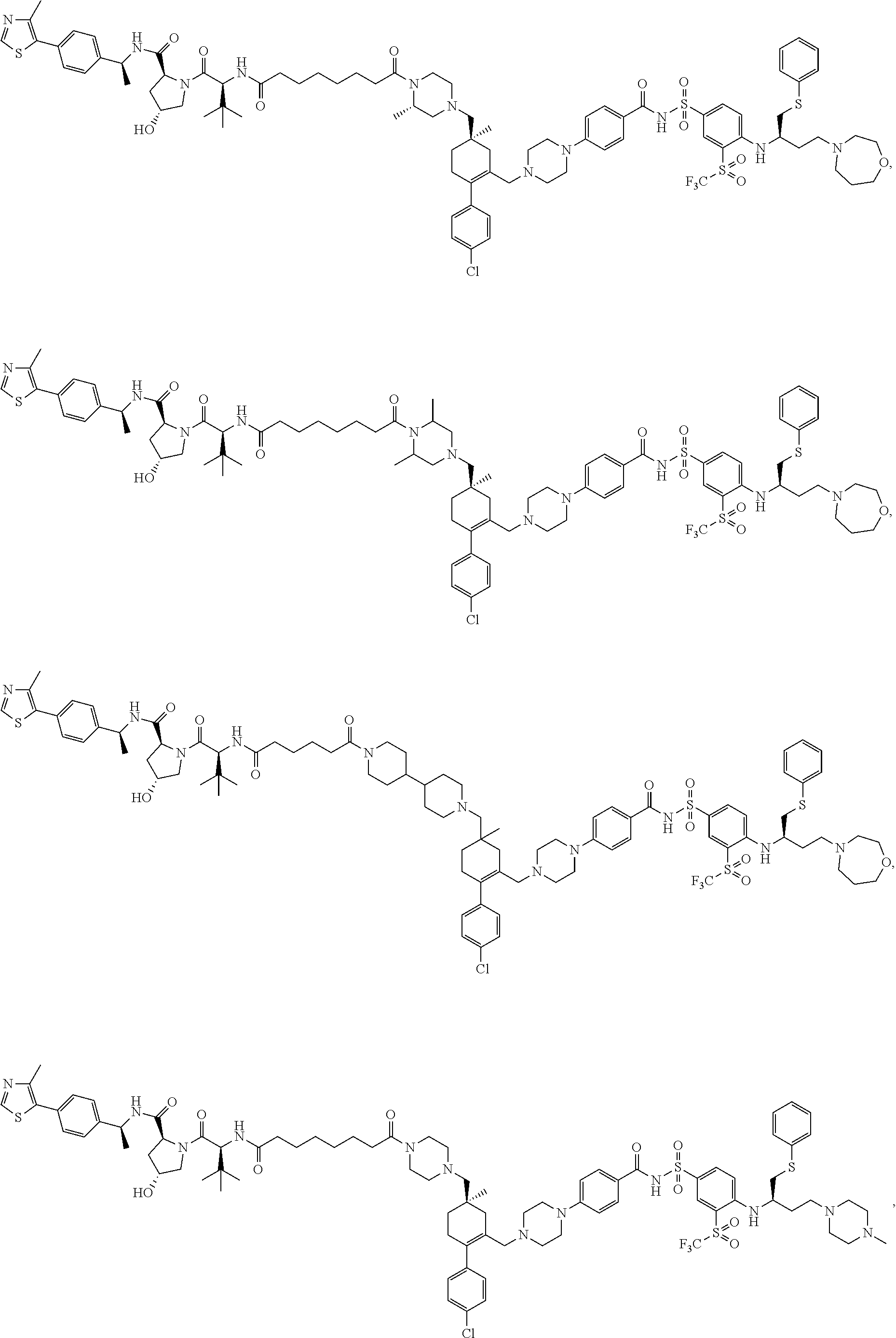 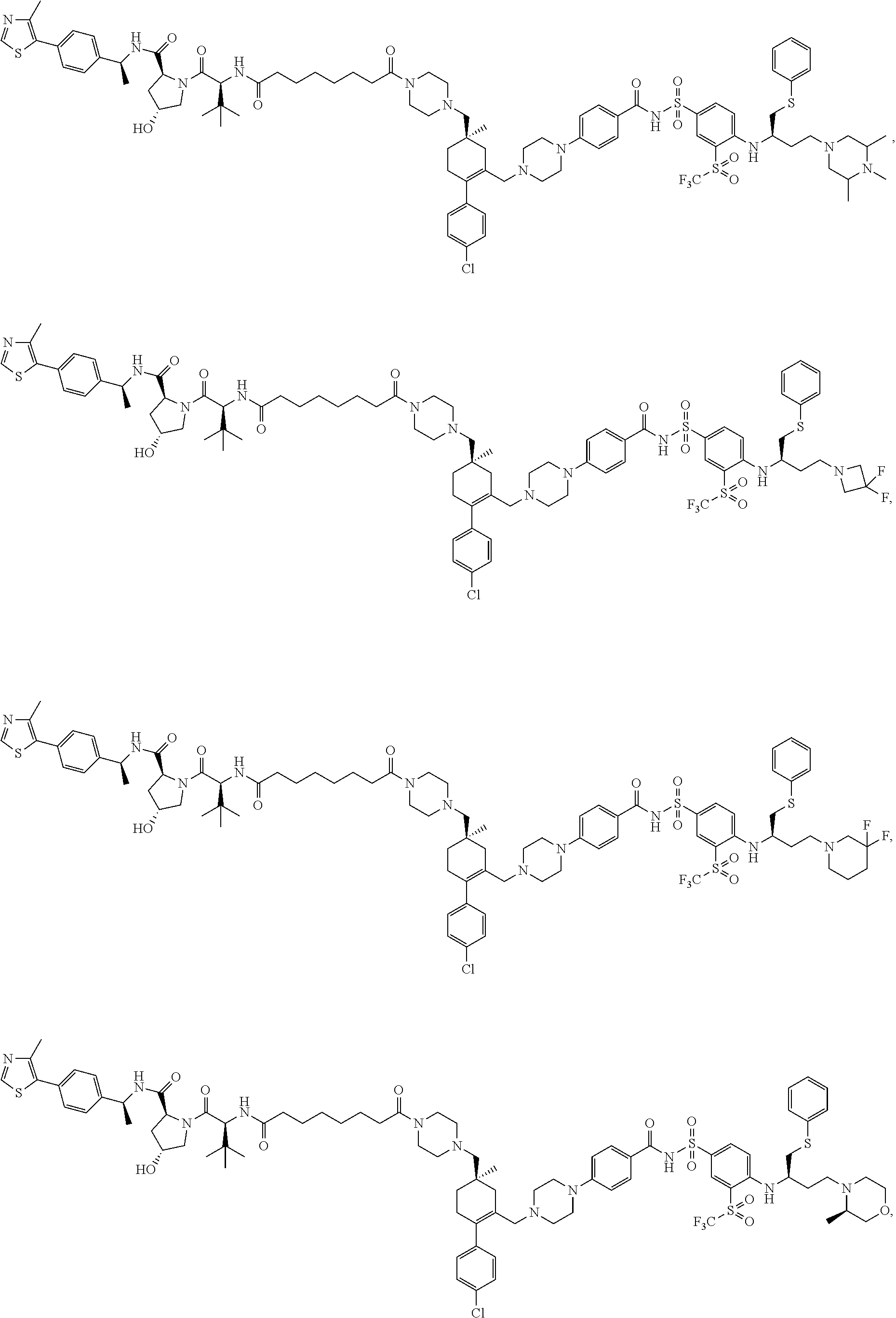 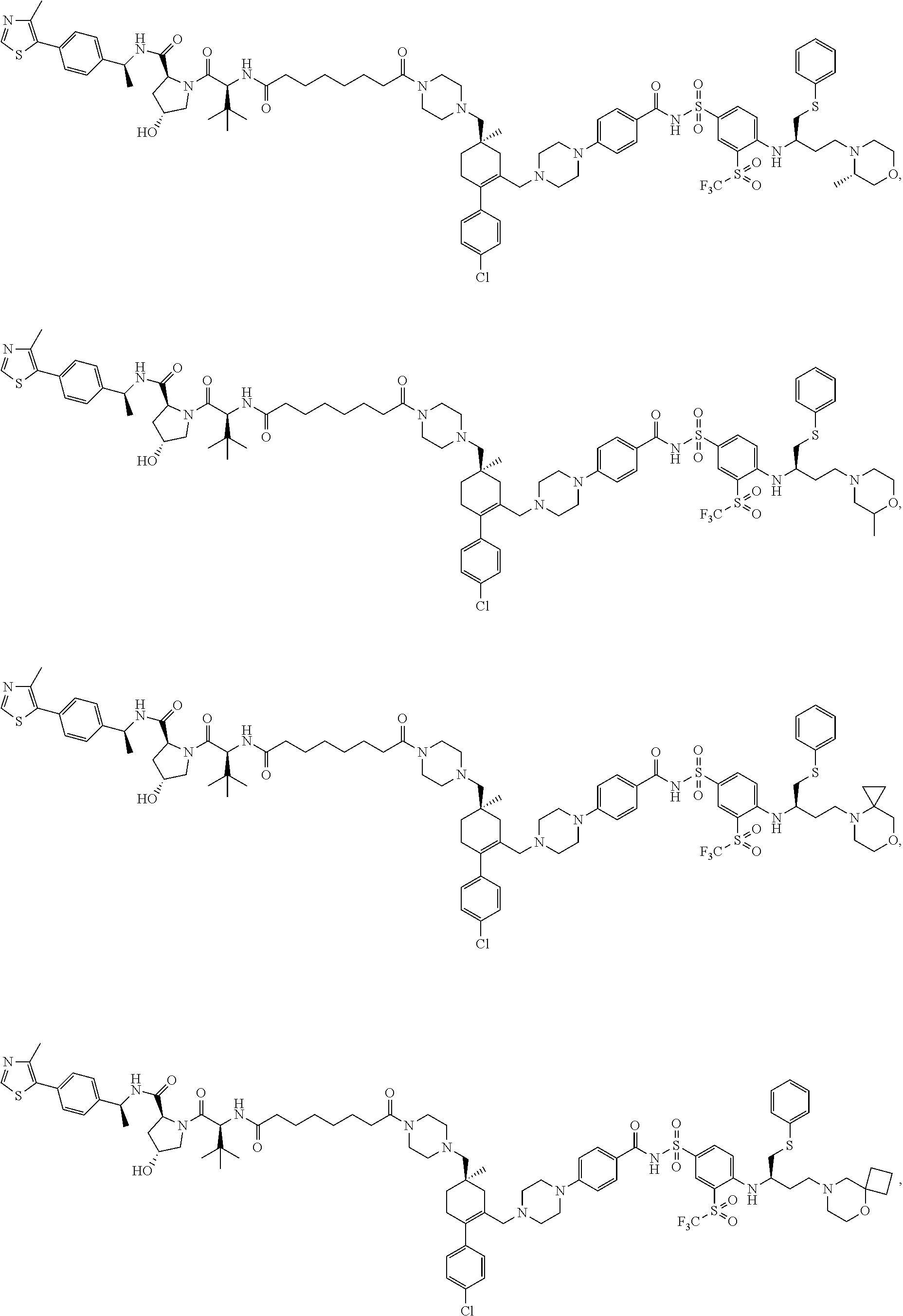 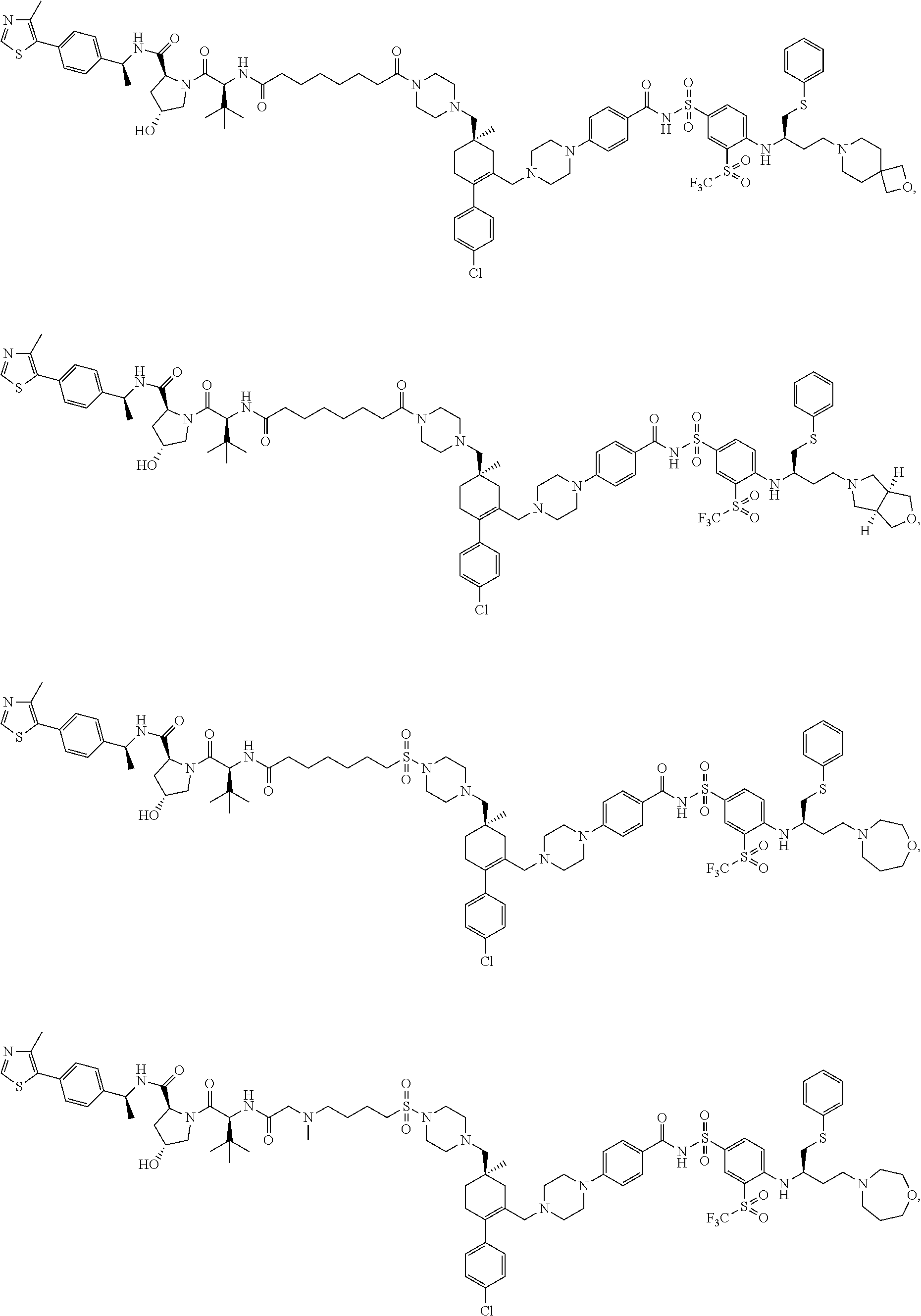 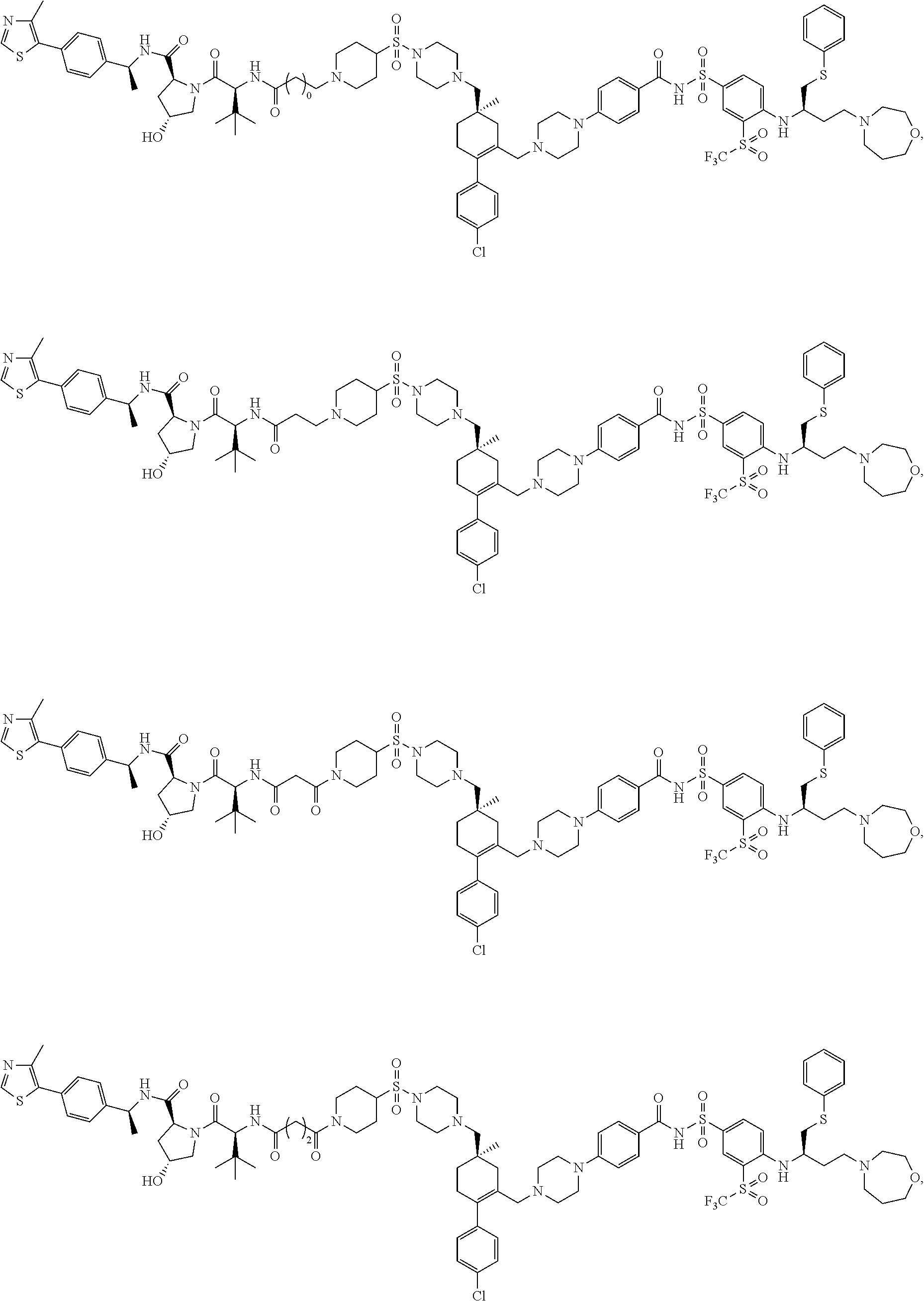 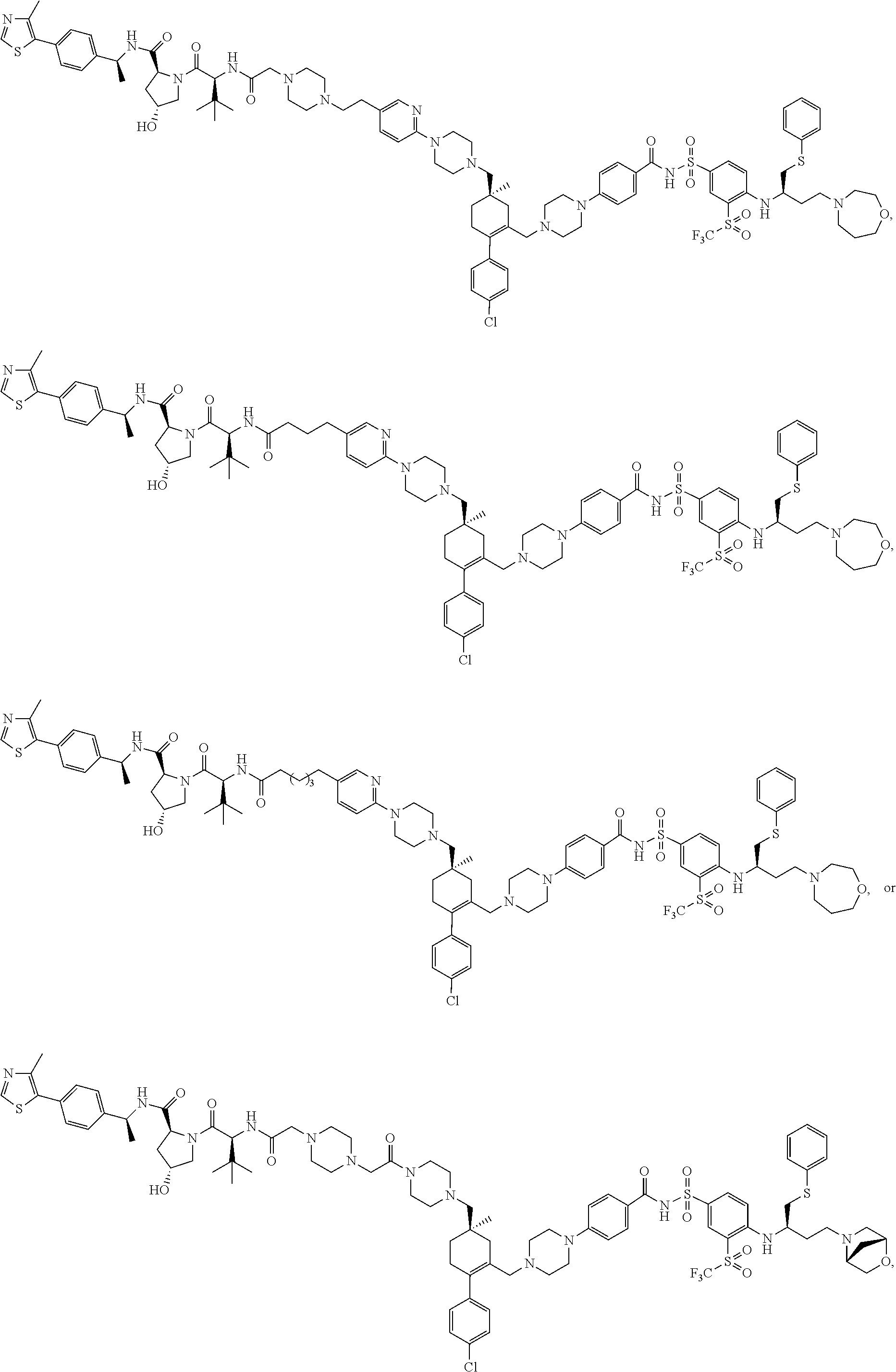
or a pharmaceutically acceptable salt thereof.

31. The compound of claim 1, or a pharmaceutically acceptable salt thereof, wherein the compound is: 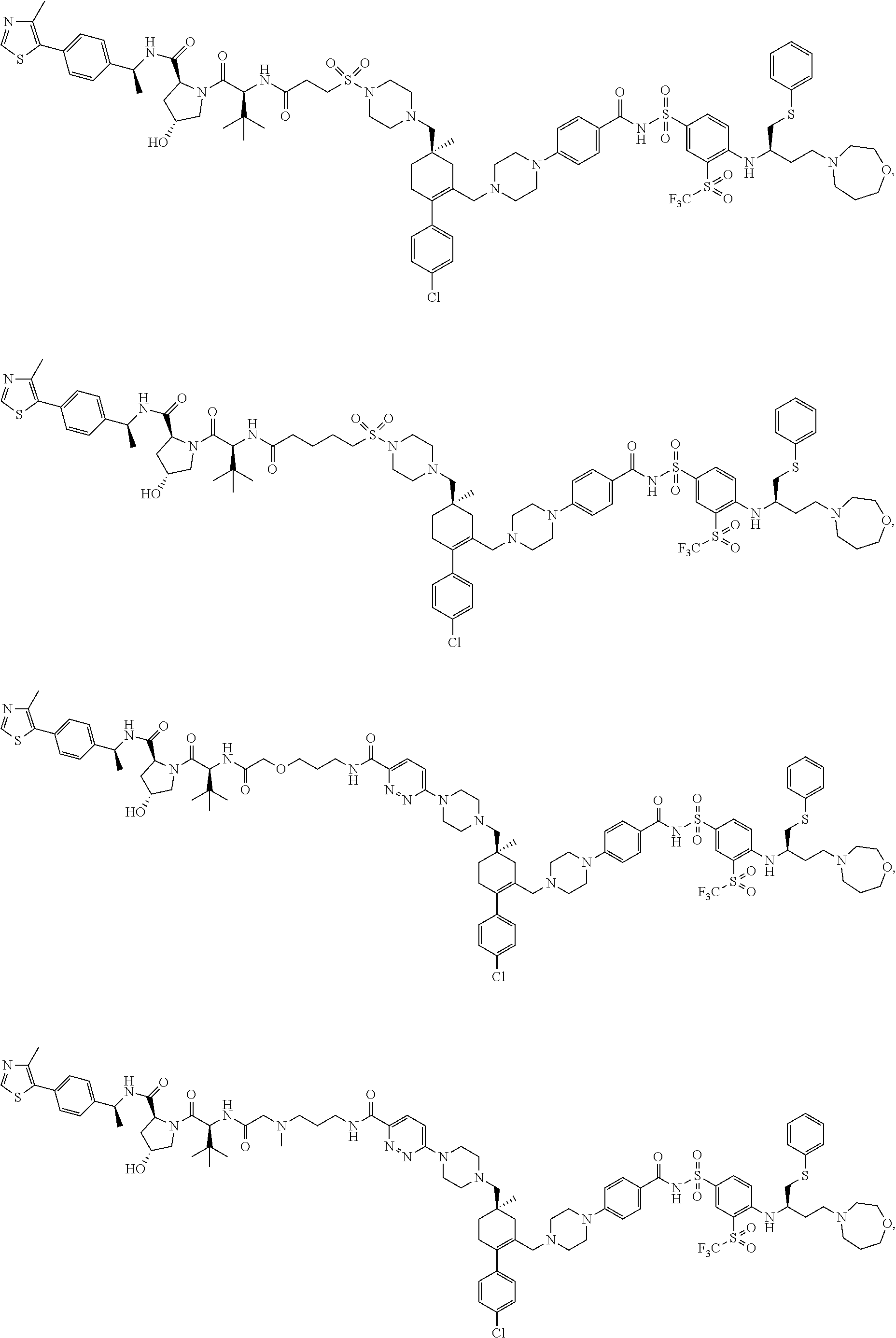 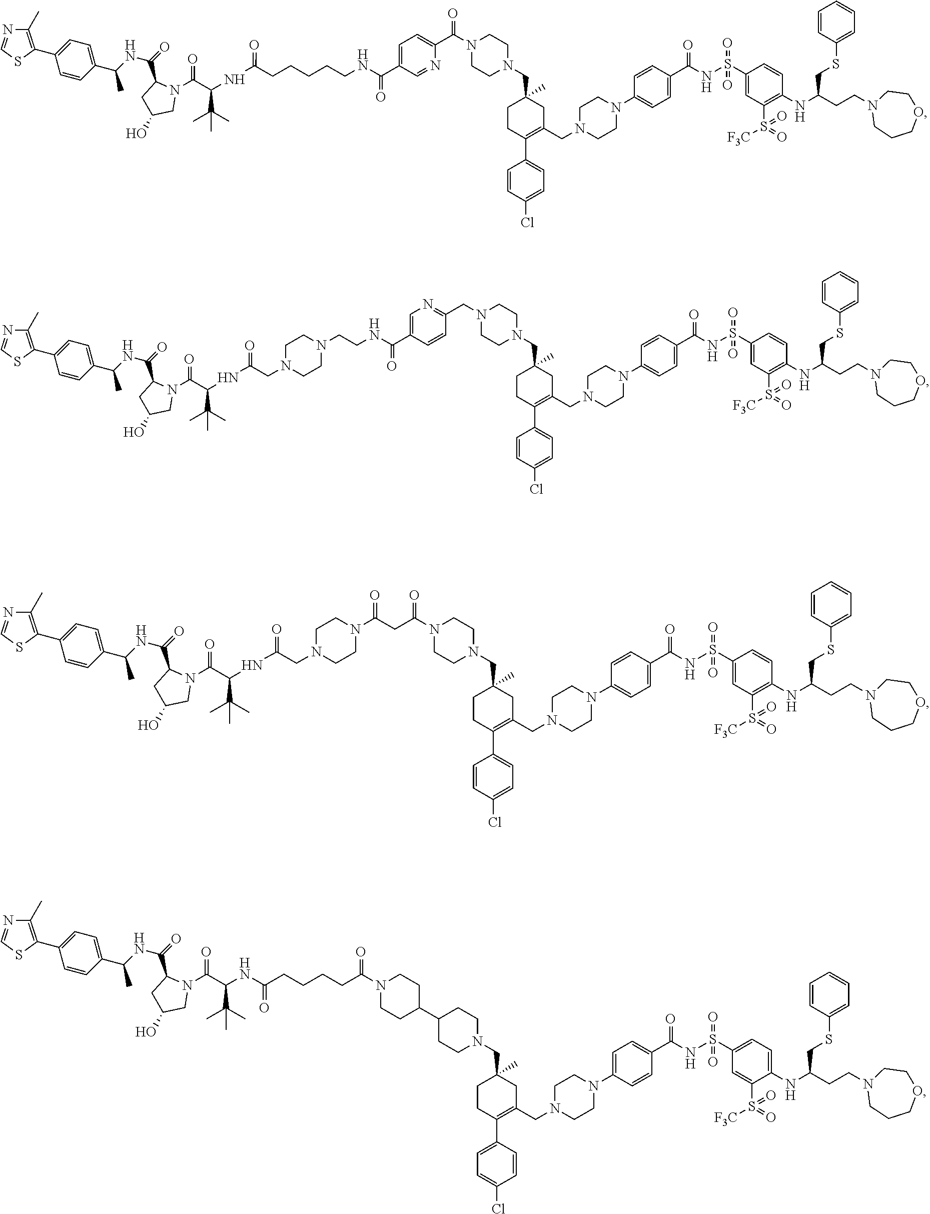 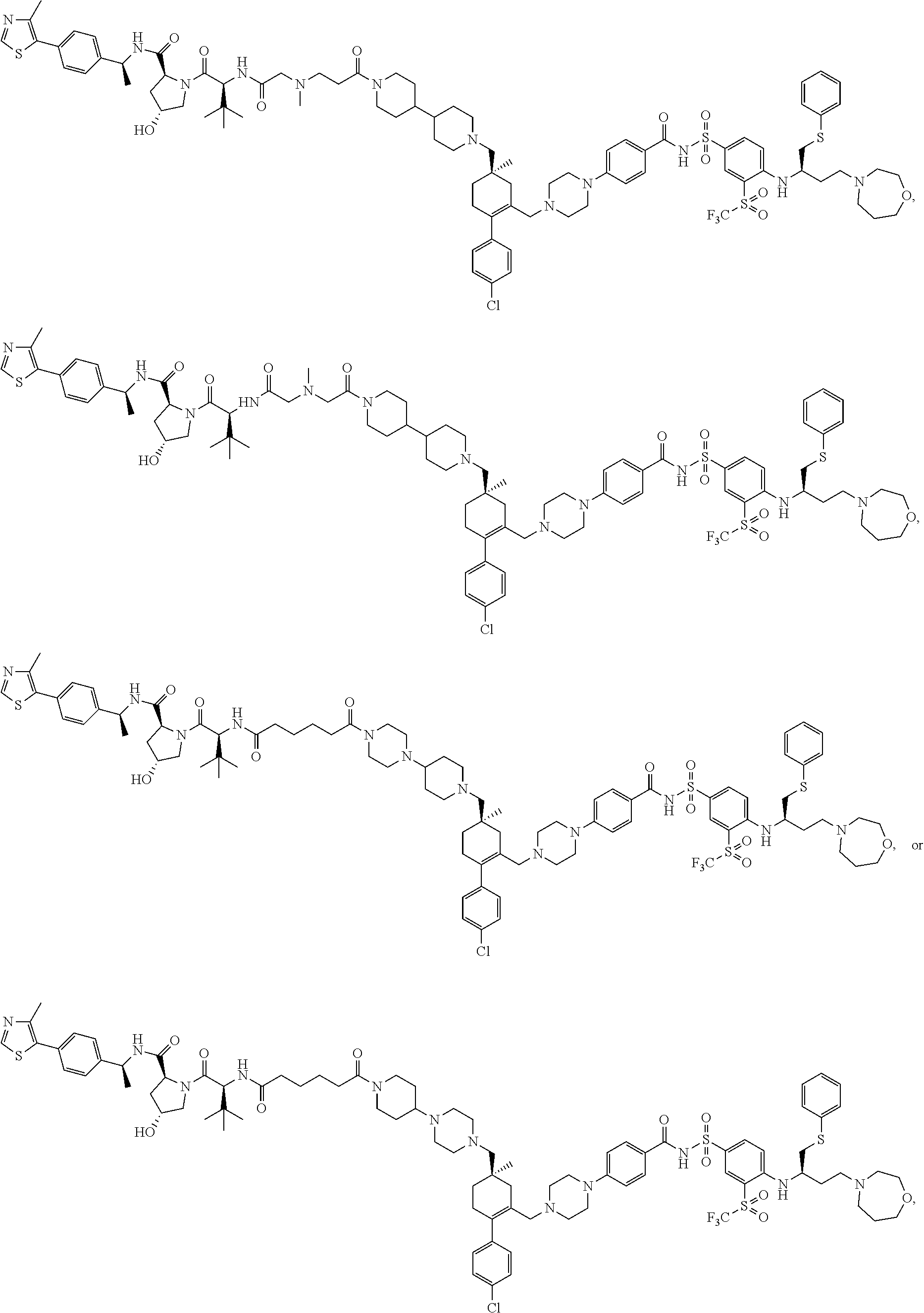
or a pharmaceutically acceptable salt thereof.

32. A compound of Formula (II), or a pharmaceutically acceptable salt thereof: 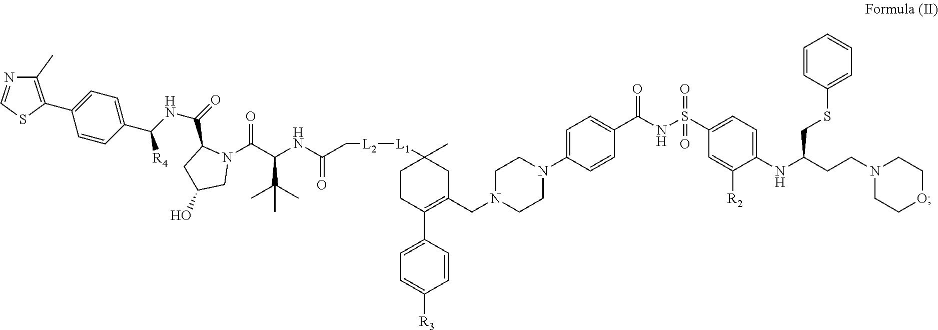
wherein R₂ is NO₂, SO₂CF₃, or SO₂CF₂Cl;
R₃ is Cl, F, CF₂H, CFH₂, or CF₃;
R₄ is H or CH₃;
L₁ is 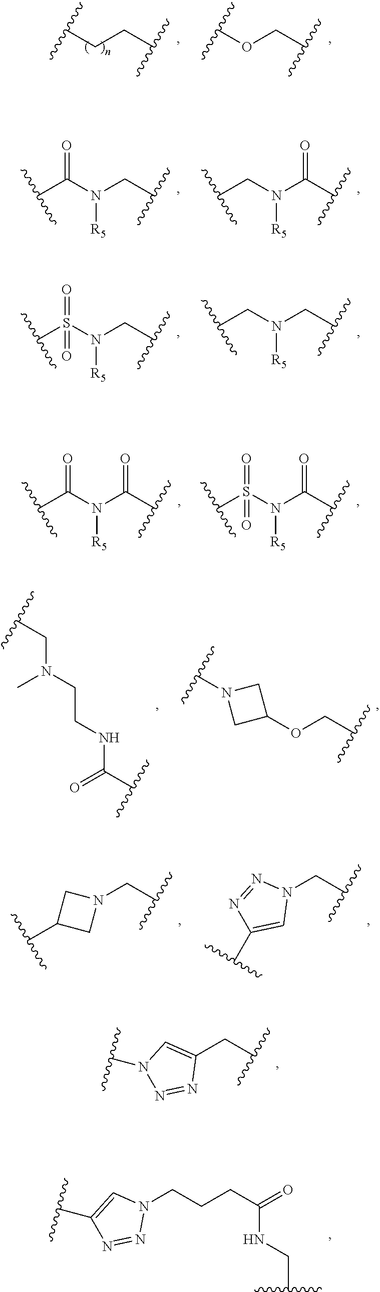 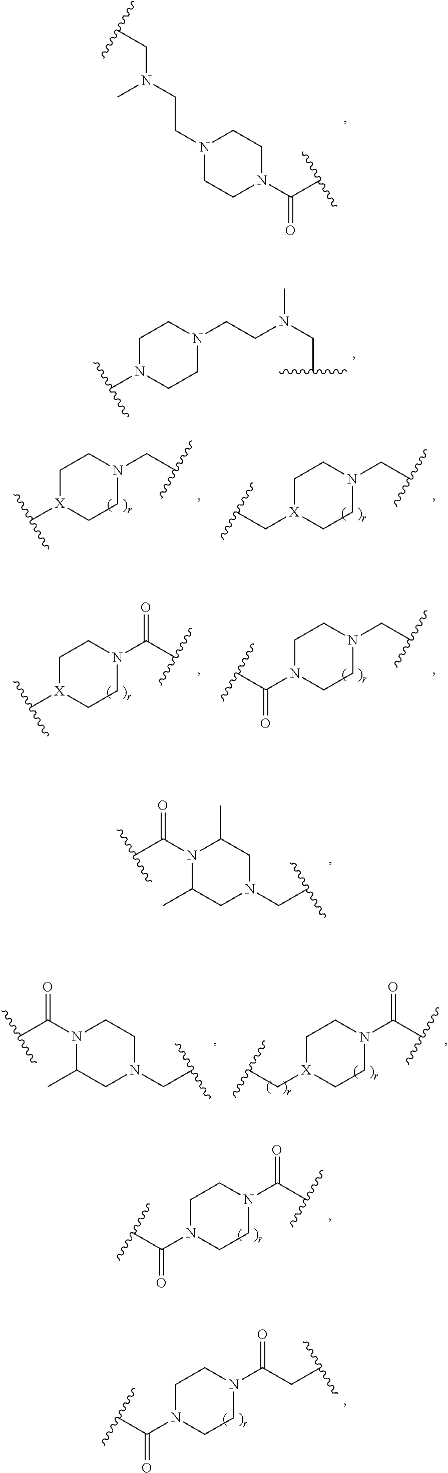 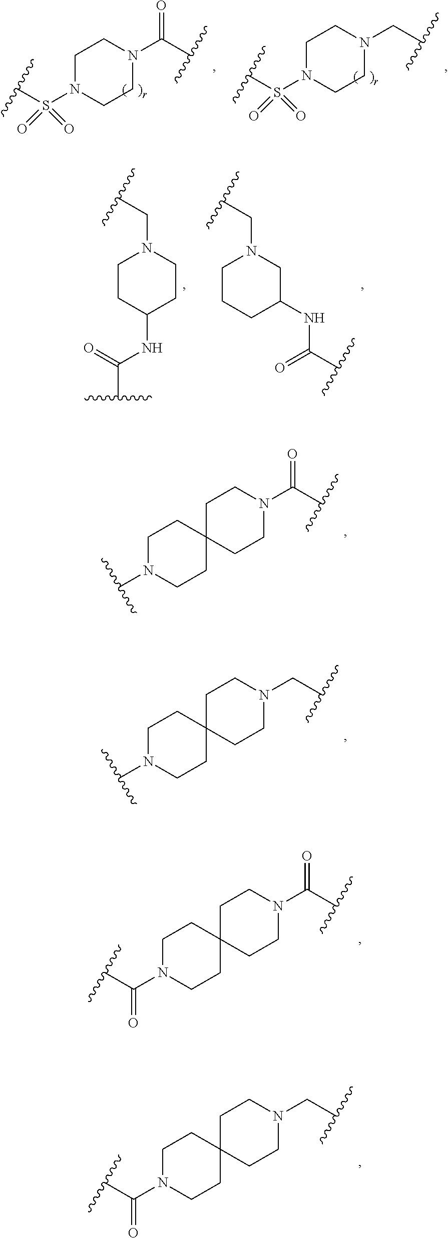 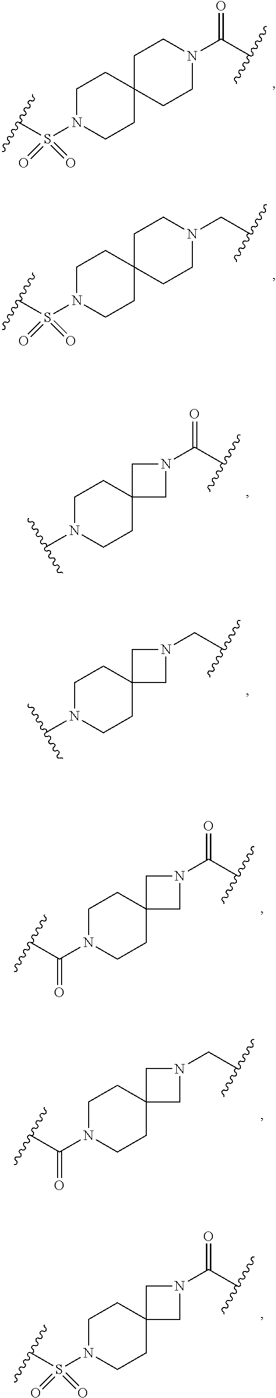 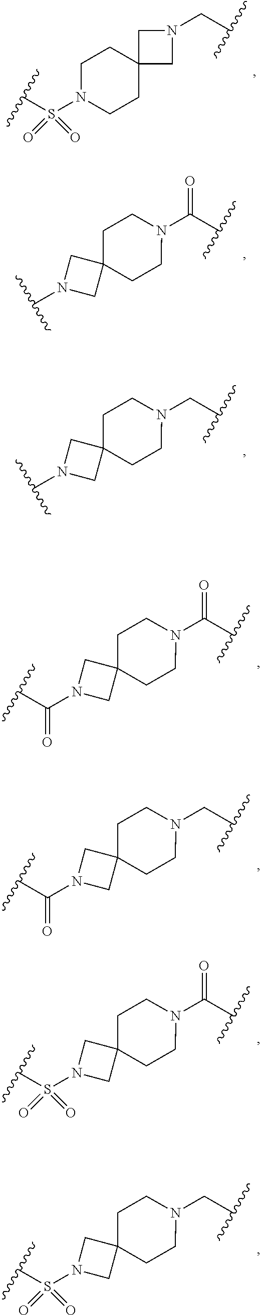 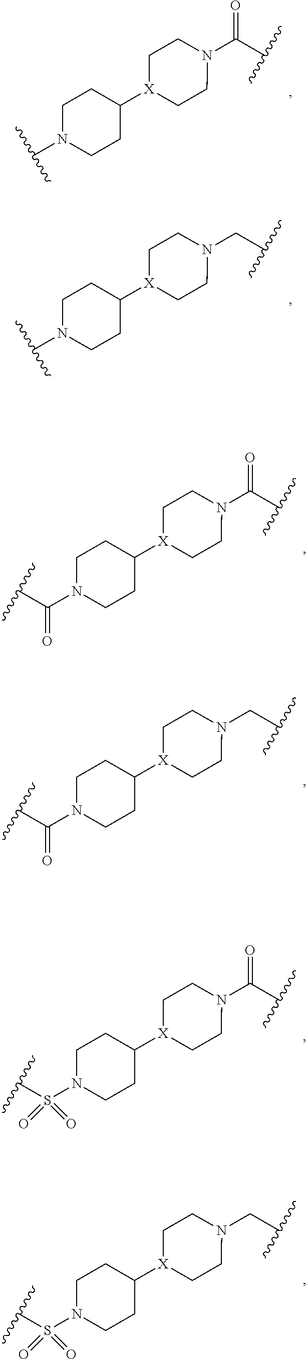 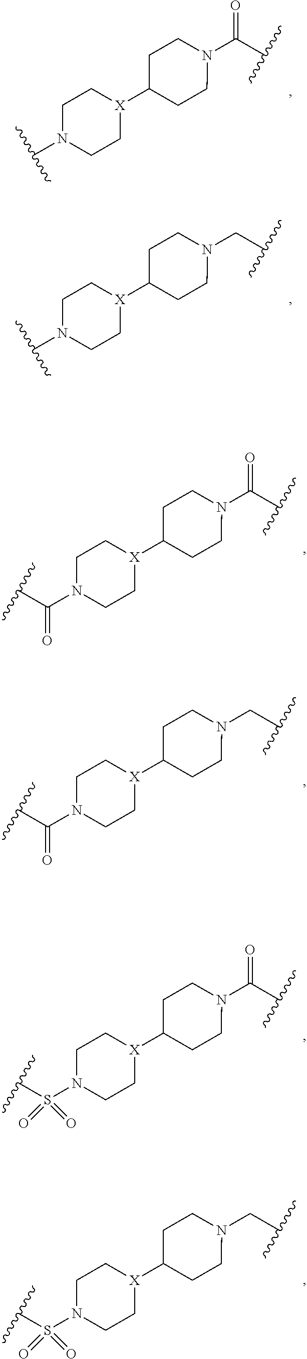 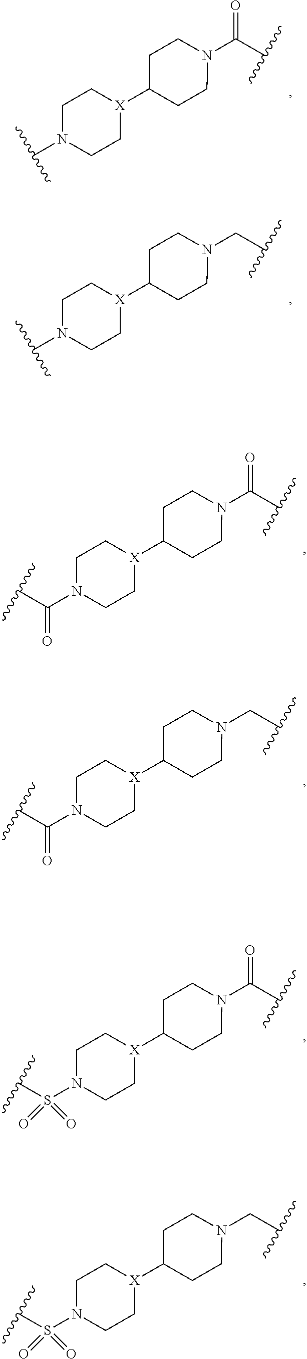 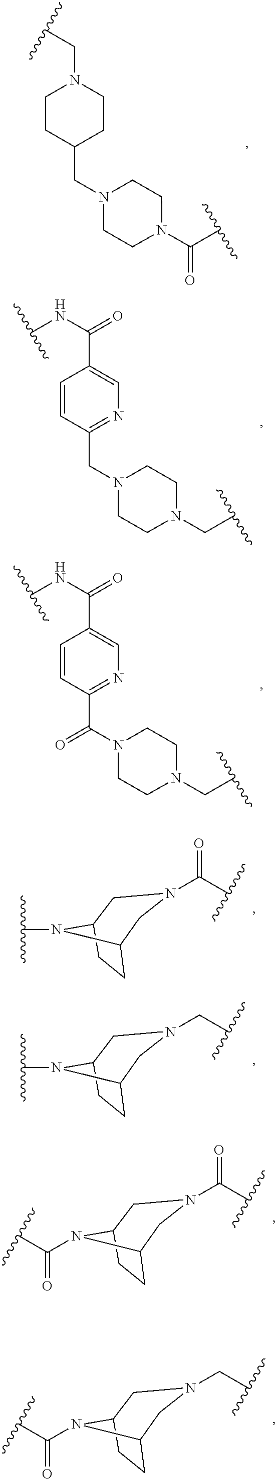 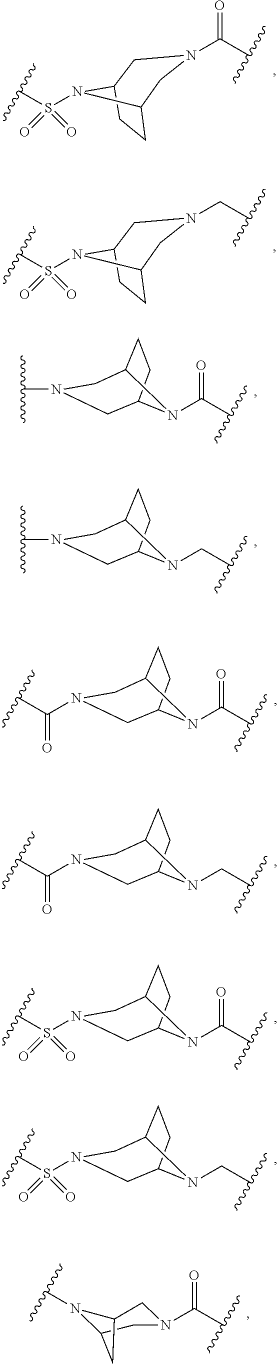 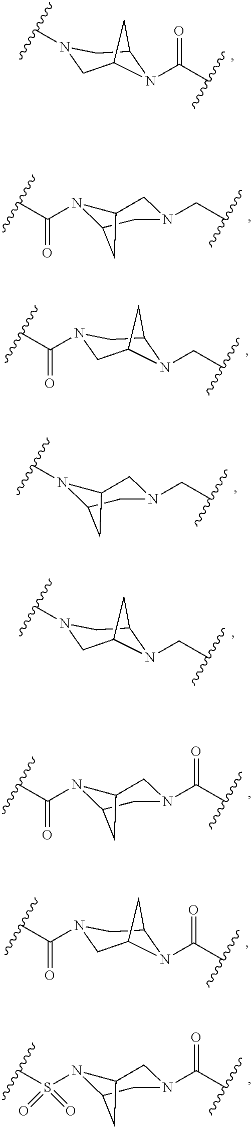 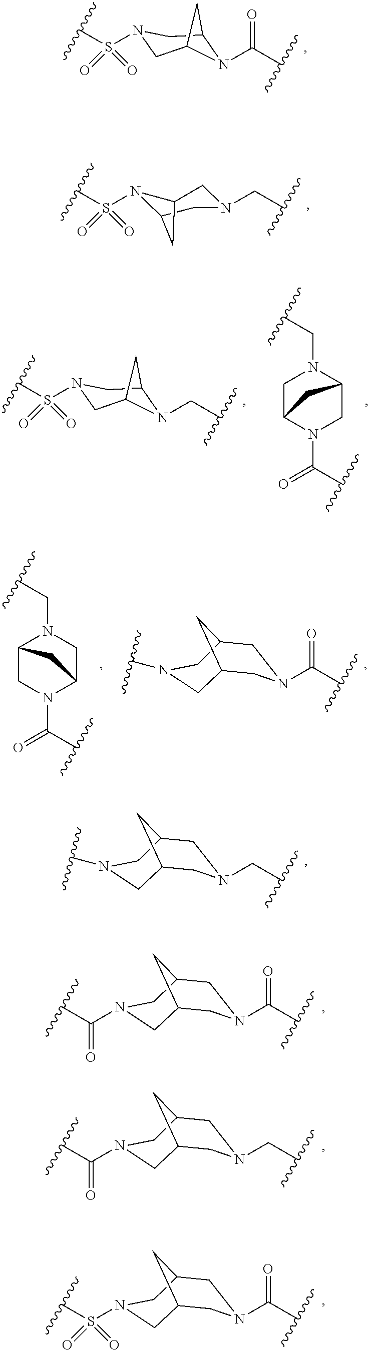 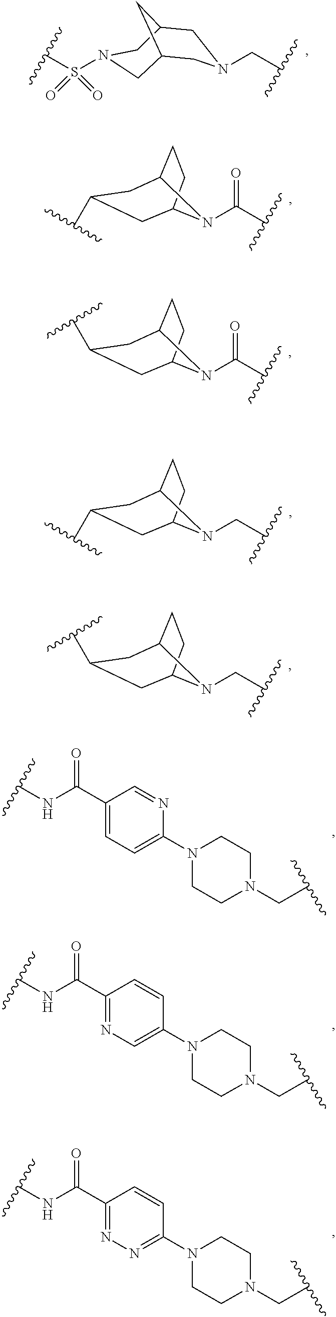 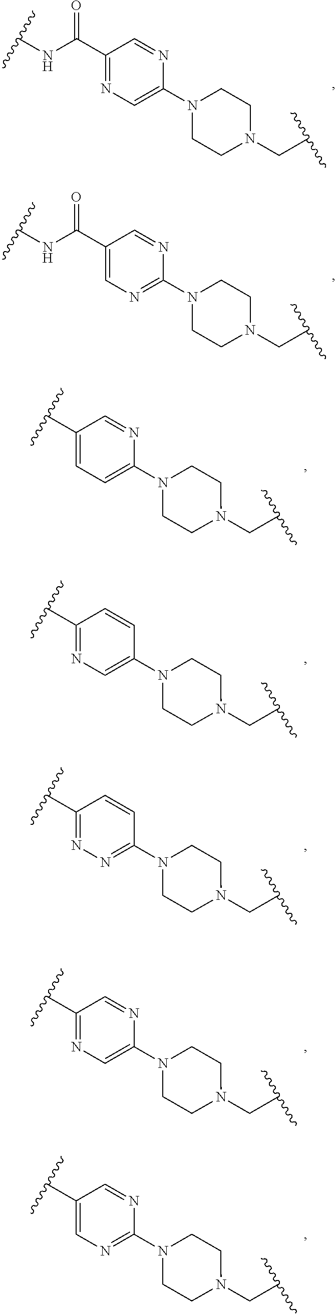 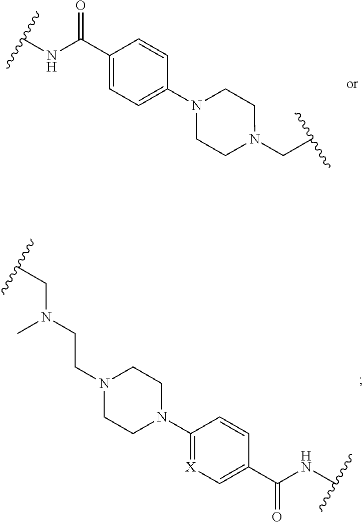
L₂ is a bond, 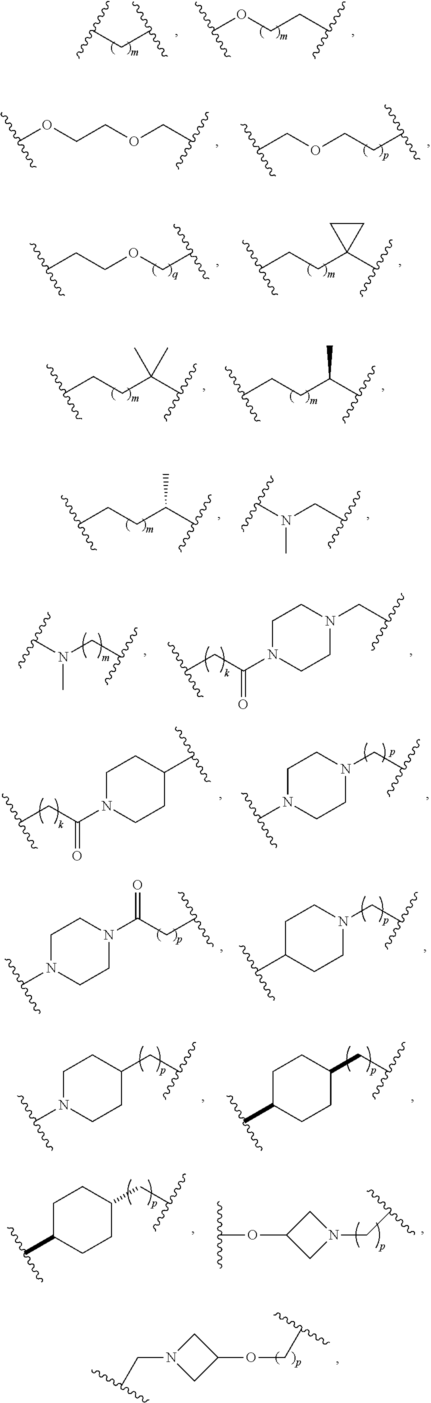 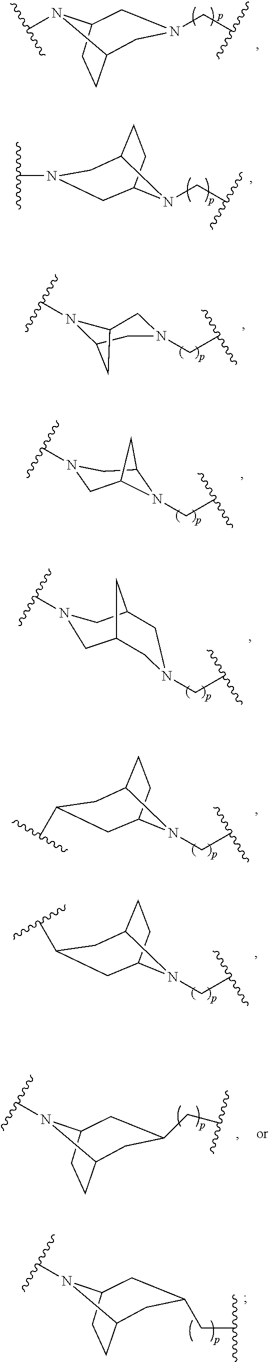
each X is independently CH or N
each k is independently 0, 1, 2, 3, 4, 5, or 6;
each m is independently 2, 3, 4, 5, 6, or 7;
each n is independently 02, 3, 4, 5, 6, 7, 8, 9, or 10;
each p is independently 0, 1, 2, 3, or 4;
each q is independently 1, 2, or 3;
each r is independently 1 or 2; and
R₅ is independently H, optionally substituted alkyl, or optionally substituted cycloalkyl; provided that when R₂ is SO₂CF₃ and L₁ is 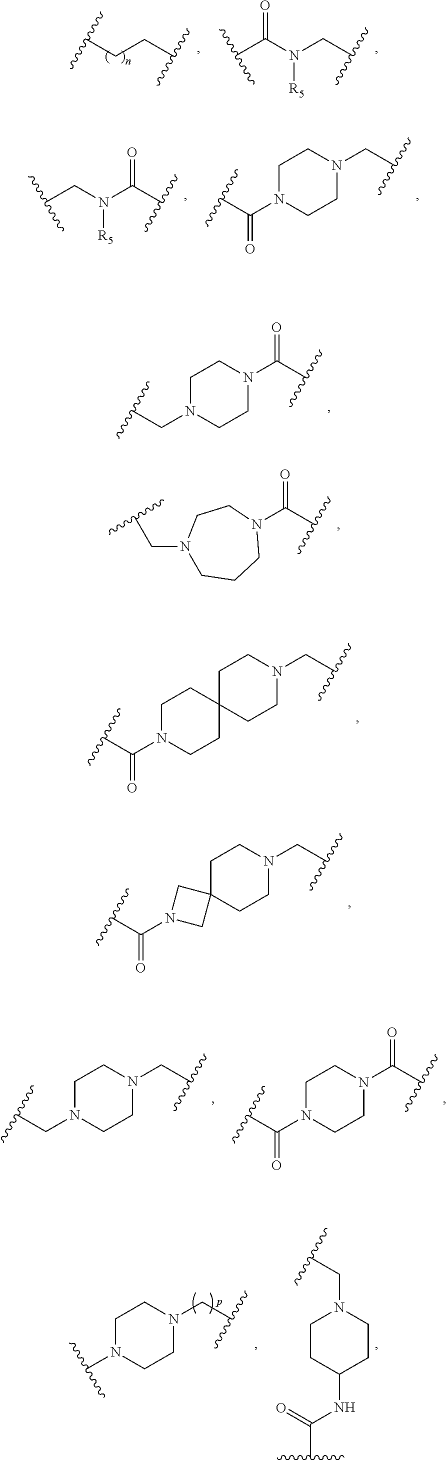 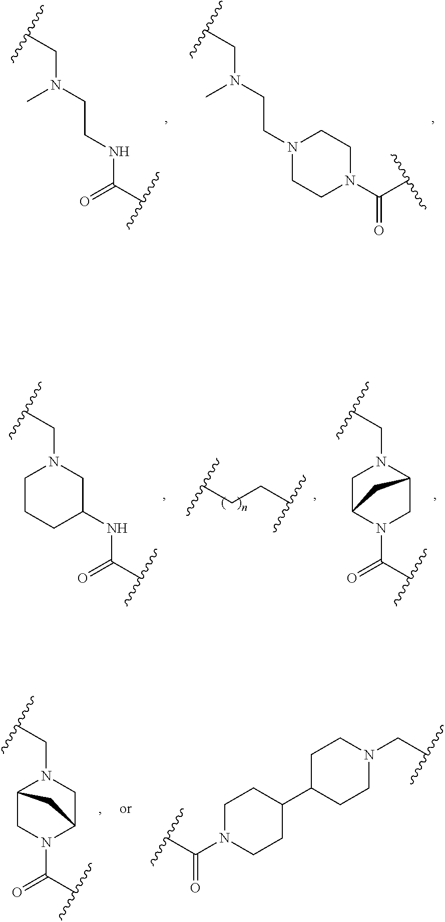
then L₂ is not a bond,

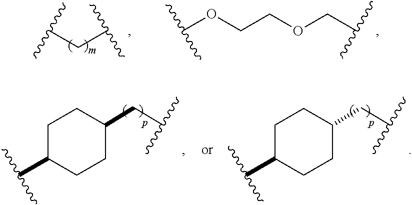

33. The compound of claim 32, or a pharmaceutically acceptable salt thereof, wherein:
L₁ is 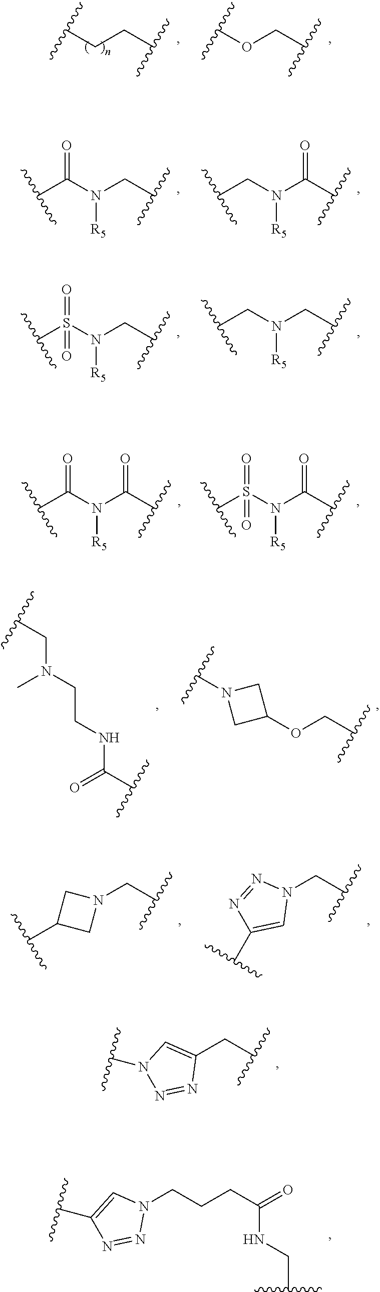 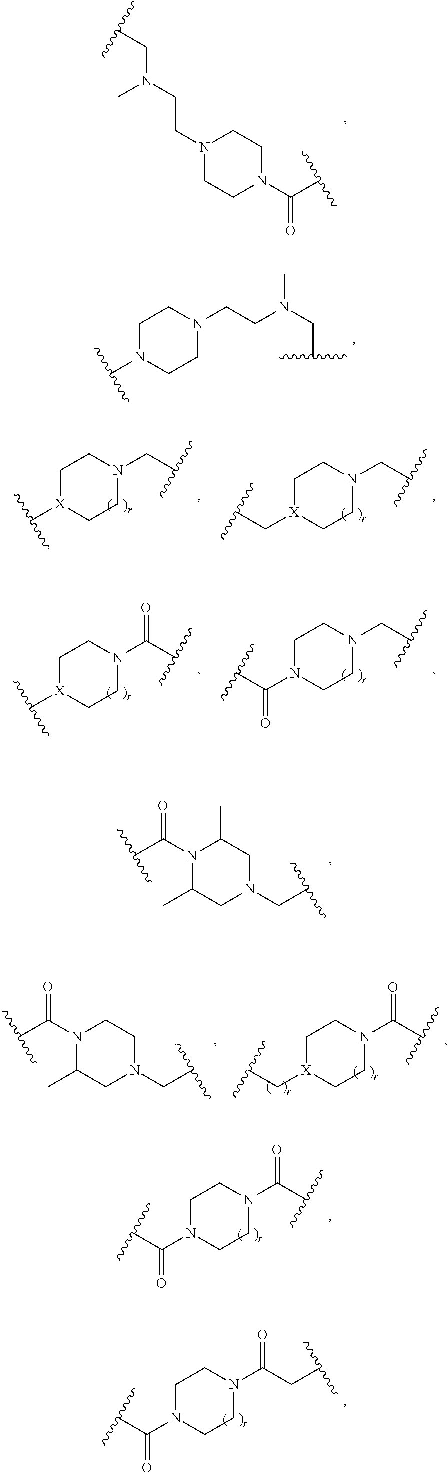 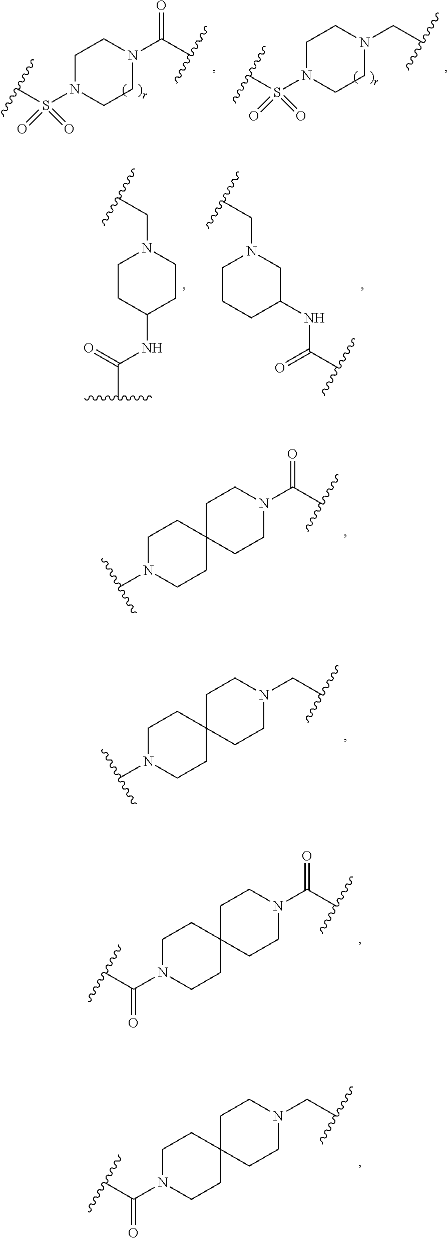 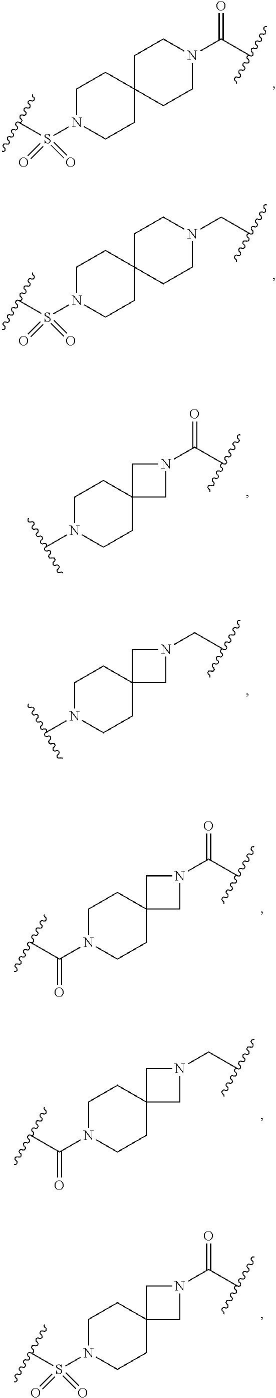 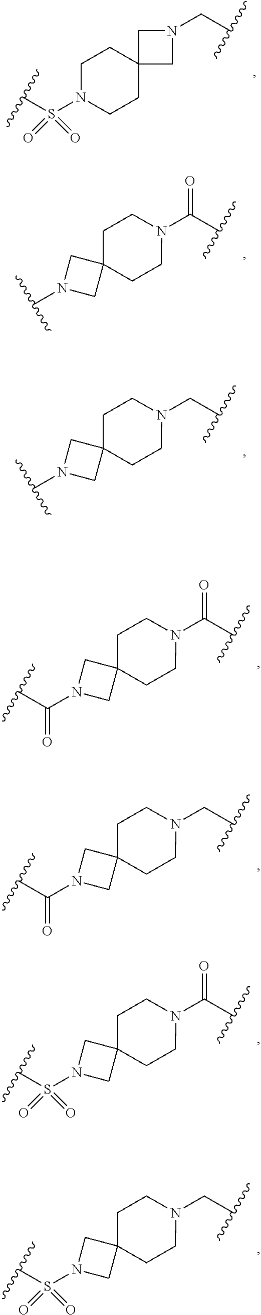 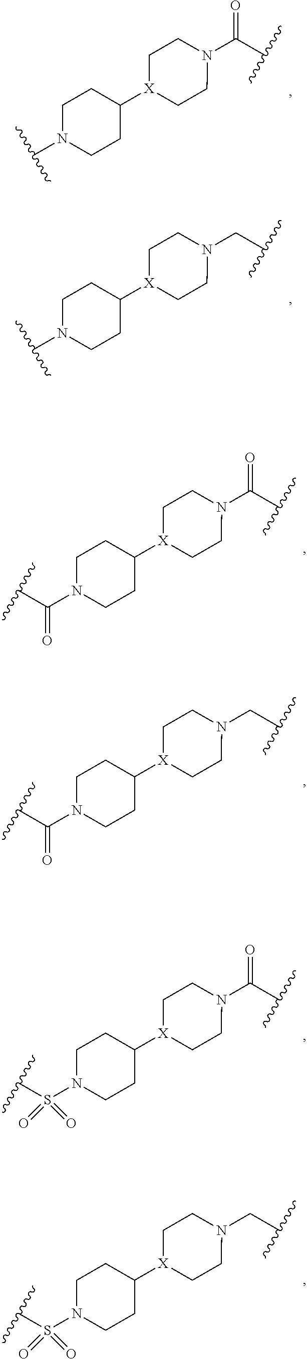 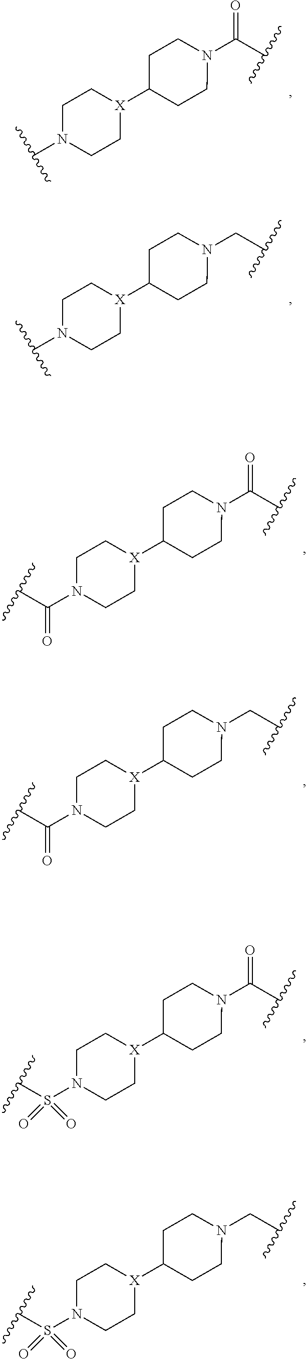 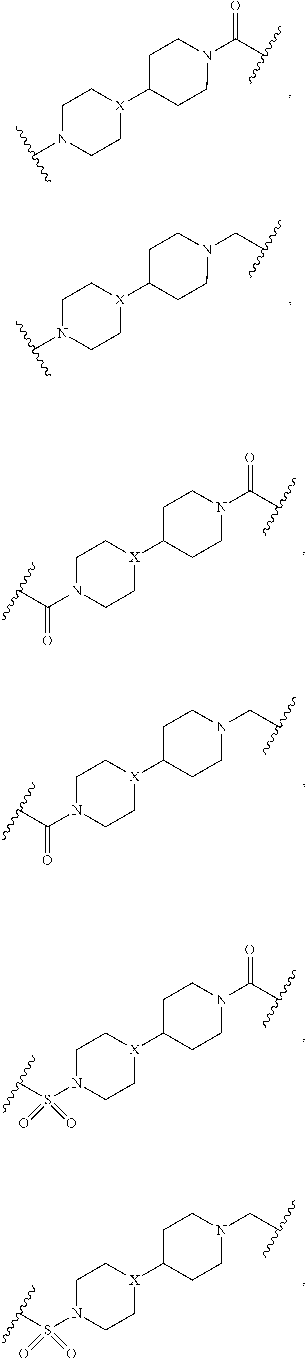 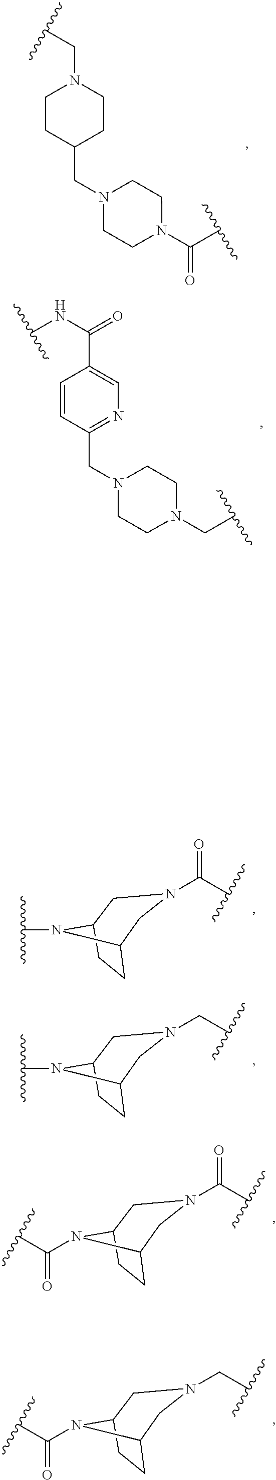 [SMILES: *CN1CCC(CN2CCN(C(*)=O)CC2)CC1 ; *CN1CCN(Cc2ccc(C(=O)N*)cn2)CC1 ; *C(=O)N1CC2CCC(C1)N2* ; *CN1CC2CCC(C1)N2* ; *C(=O)N1CC2CCC(C1)N2C(*)=O ; *CN1CC2CCC(C1)N2C(*)=O] 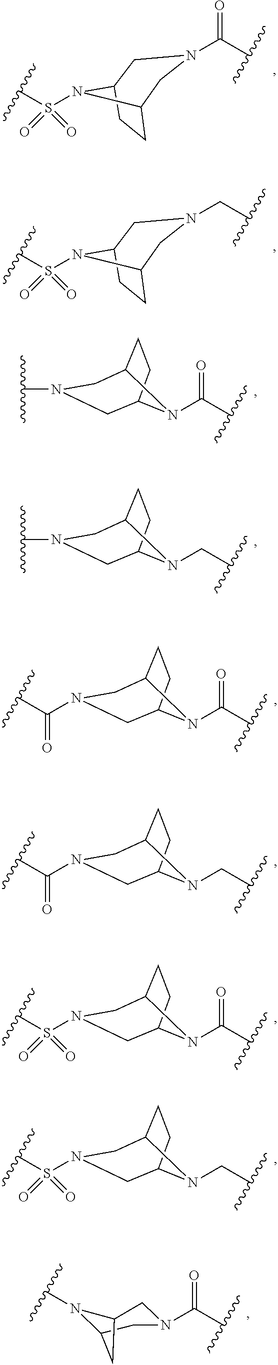 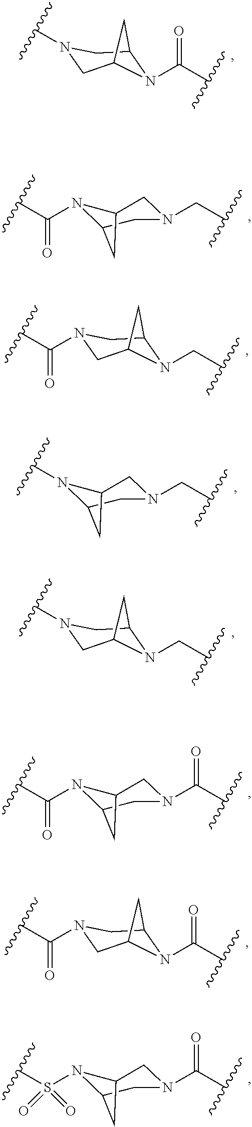 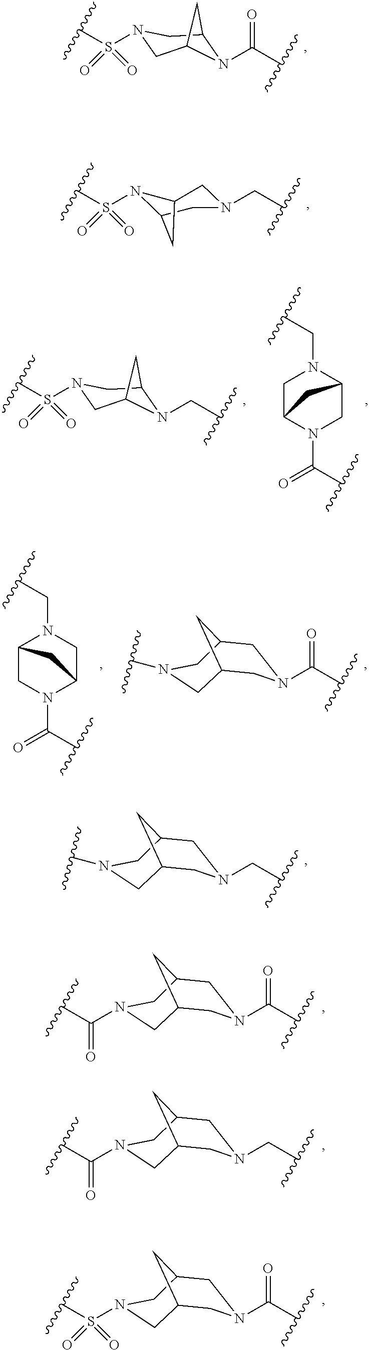 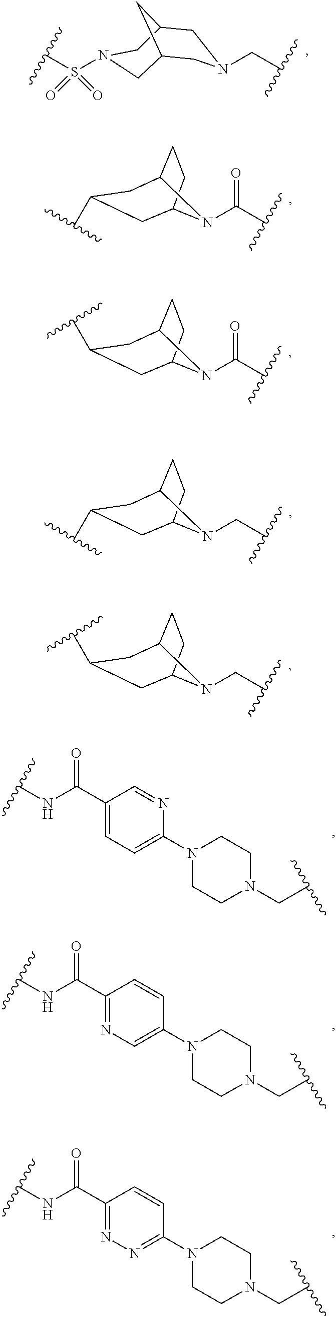 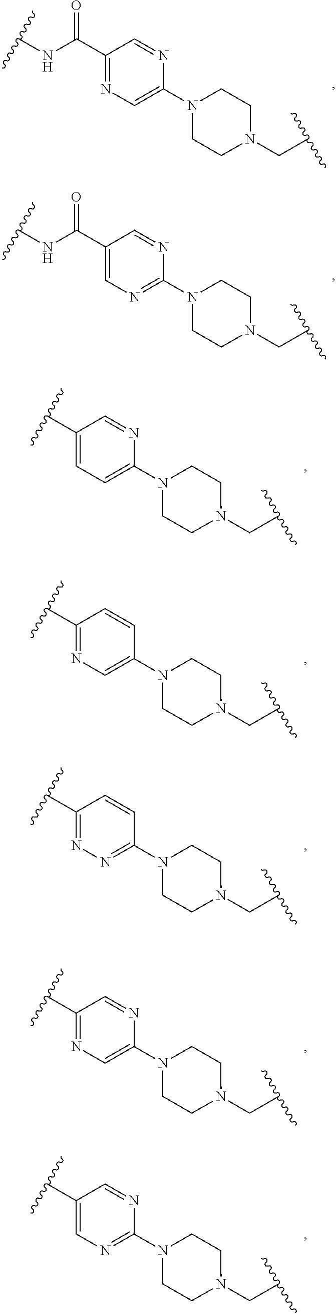 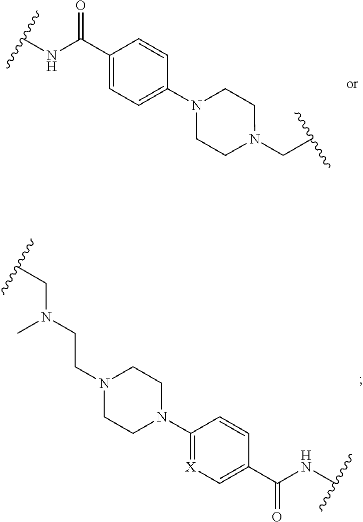
and
L₂ is a bond,

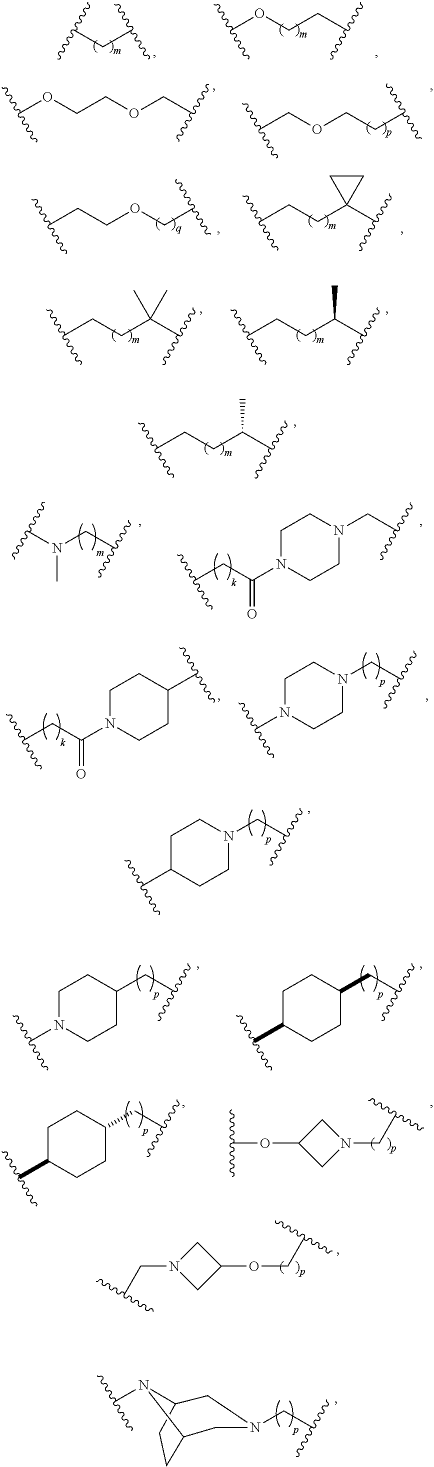

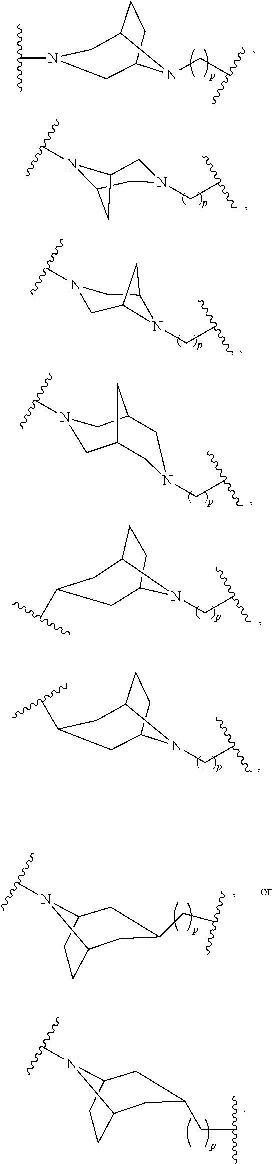

34. The compound of claim 32 or 33, or a pharmaceutically acceptable salt thereof, wherein the compound is of formula:

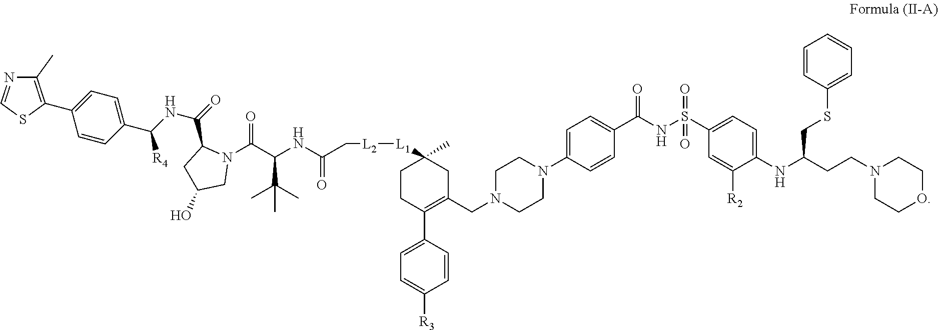

35. The compound of any one of claims 32-34, or a pharmaceutically acceptable salt thereof, wherein L₁ is

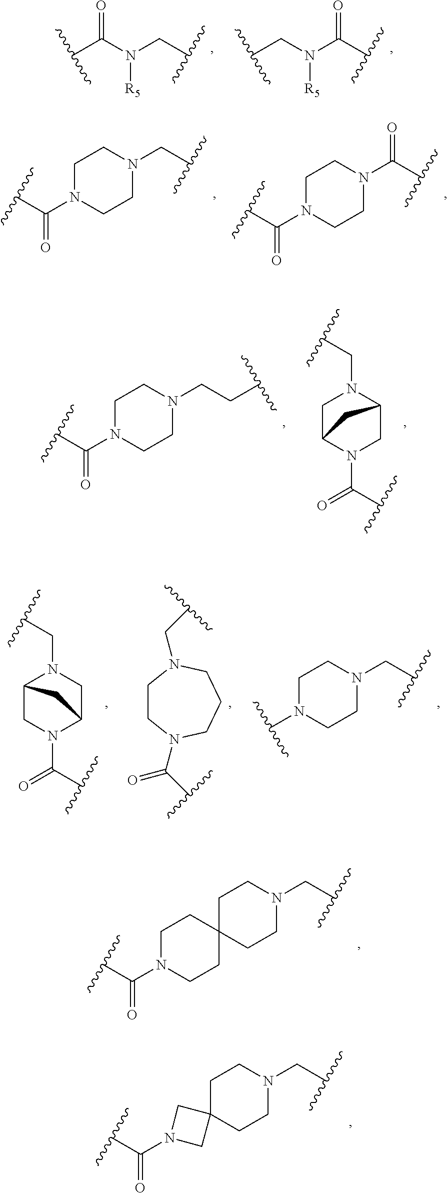

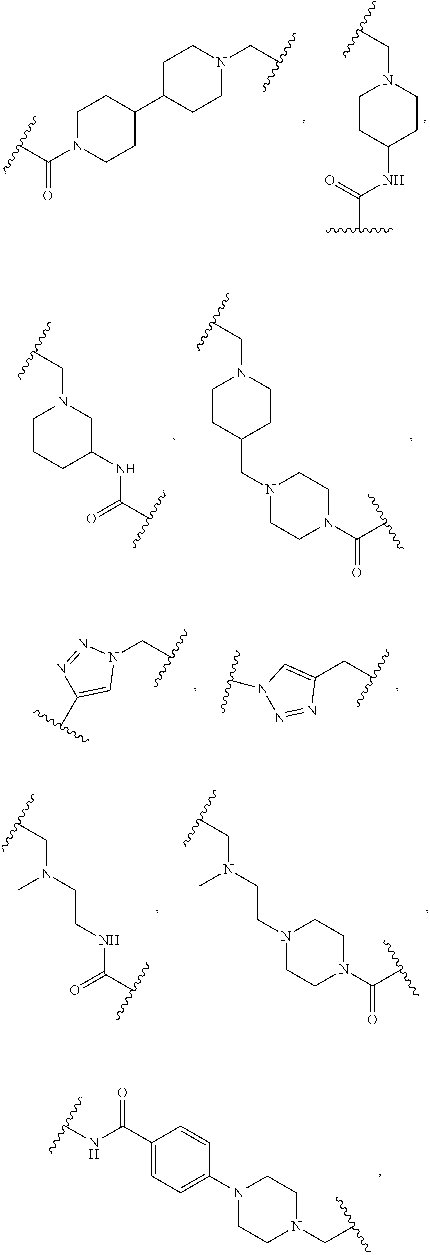

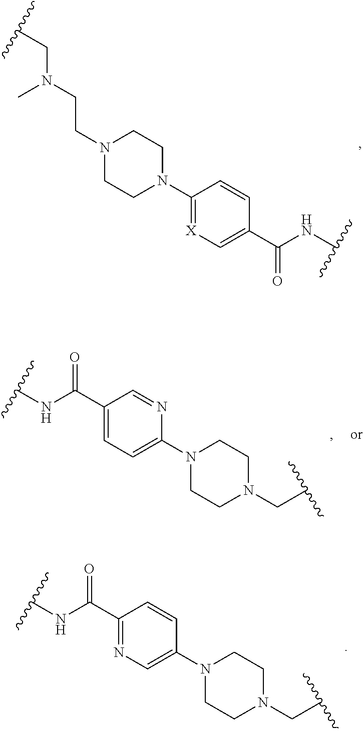

36. The compound of claim 35, or a pharmaceutically acceptable salt thereof, wherein L₁ is

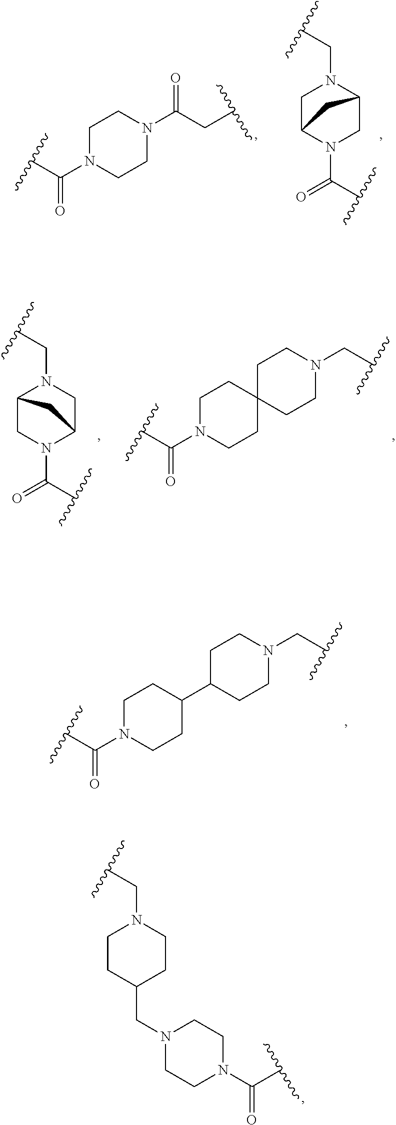 [SMILES: *CC(=O)N1CCN(C(*)=O)CC1 ; *CN1C[C@@H]2C[C@H]1CN2C(*)=O ; *CN1C[C@H]2C[C@@H]1CN2C(*)=O ; *CN1CCC2(CC1)CCN(C(*)=O)CC2 ; *CN1CCC(C2CCN(C(*)=O)CC2)CC1 ; *CN1CCC(CN2CCN(C(*)=O)CC2)CC1]

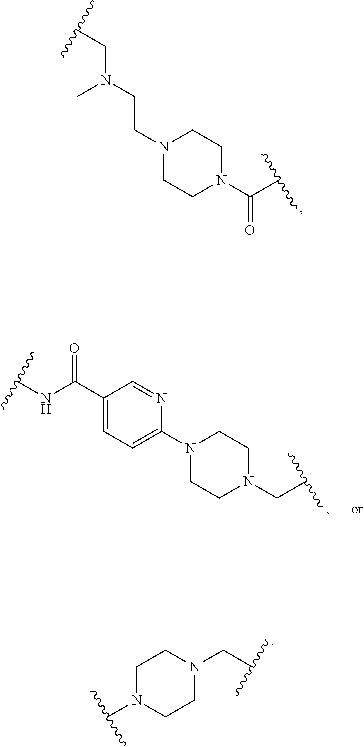 [SMILES: *CN(C)CCN1CCN(C(*)=O)CC1 ; *CN1CCN(c2ccc(C(=O)N*)cn2)CC1 ; *CN1CCN(*)CC1]

37. The compound of any one of claims 32-36, or a pharmaceutically acceptable salt thereof, wherein L₂ is

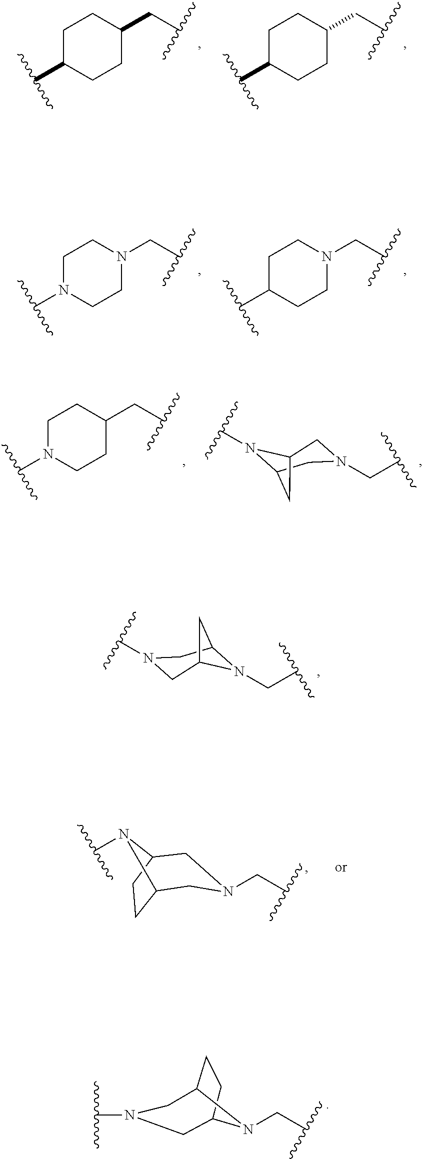

38. The compound of claim 37, or a pharmaceutically acceptable salt thereof, wherein L₂ is

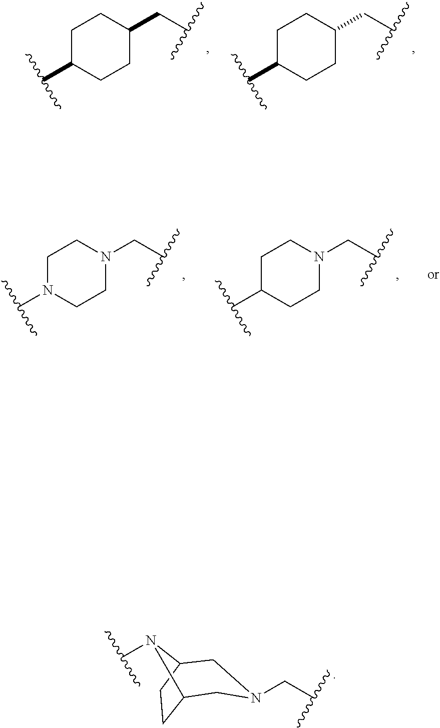 [SMILES: *C[C@H]1CC[C@@H](*)CC1 ; *C[C@H]1CC[C@H](*)CC1 ; *CN1CCN(*)CC1 ; *CN1CCC(*)CC1 ; *CN1CC2CCC(C1)N2*]

39. The compound of any one of claims 32-34 and 37-38, or a pharmaceutically acceptable salt thereof, wherein L₁ is 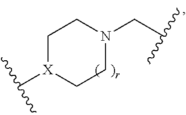
X is N, and r is 1.

40. The compound of claim 32, or a pharmaceutically acceptable salt thereof, wherein L₁ is 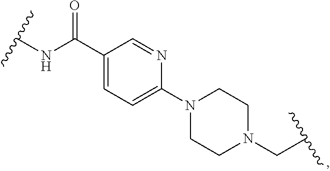 [SMILES: *CN1CCN(c2ccc(C(=O)N*)cn2)CC1]
and L₂ is a bond or

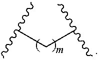

41. The compound of claim 32, or a pharmaceutically acceptable salt thereof, wherein L₁ is 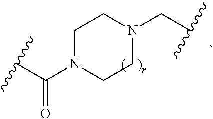
and L₂ is

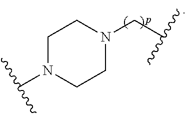

42. The compound of any one of claims 32-41, or a pharmaceutically acceptable salt thereof, wherein R₂ is NO₂.

43. The compound of any one of claims 32-41, or a pharmaceutically acceptable salt thereof, wherein R₂ is SO₂CF₃.

44. The compound of any one of claims 32-43, or a pharmaceutically acceptable salt thereof, wherein R₃ is F, C₁, or CF₃.

45. The compound of claim 44, or a pharmaceutically acceptable salt thereof, wherein R₃ is Cl.

46. The compound of any one of claims 32-45, or a pharmaceutically acceptable salt thereof, wherein R₄ is CH₃.

47. The compound of any one of claims 32-45, or a pharmaceutically acceptable salt thereof, wherein R₄ is H.

48. The compound of any one of claims 32-47, or a pharmaceutically acceptable salt thereof, wherein R₅ is H.

49. The compound of any one of claims 32-47, or a pharmaceutically acceptable salt thereof, wherein R₅ is optionally substituted C₁₋₆ alkyl.

50. The compound of claim 49, or a pharmaceutically acceptable salt thereof, wherein R₅ is CH₃.

51. The compound of any one of claims 32-50, or a pharmaceutically acceptable salt thereof, wherein X is N.

52. The compound of any one of claims 32-50, or a pharmaceutically acceptable salt thereof, wherein X is CH.

53. The compound of any one of claims 32-52, or a pharmaceutically acceptable salt thereof, wherein r is 1.

54. The compound of any one of claims 32-52, or a pharmaceutically acceptable salt thereof, wherein r is 2.

55. The compound of claim 32, or a pharmaceutically acceptable salt thereof, wherein the compound is: 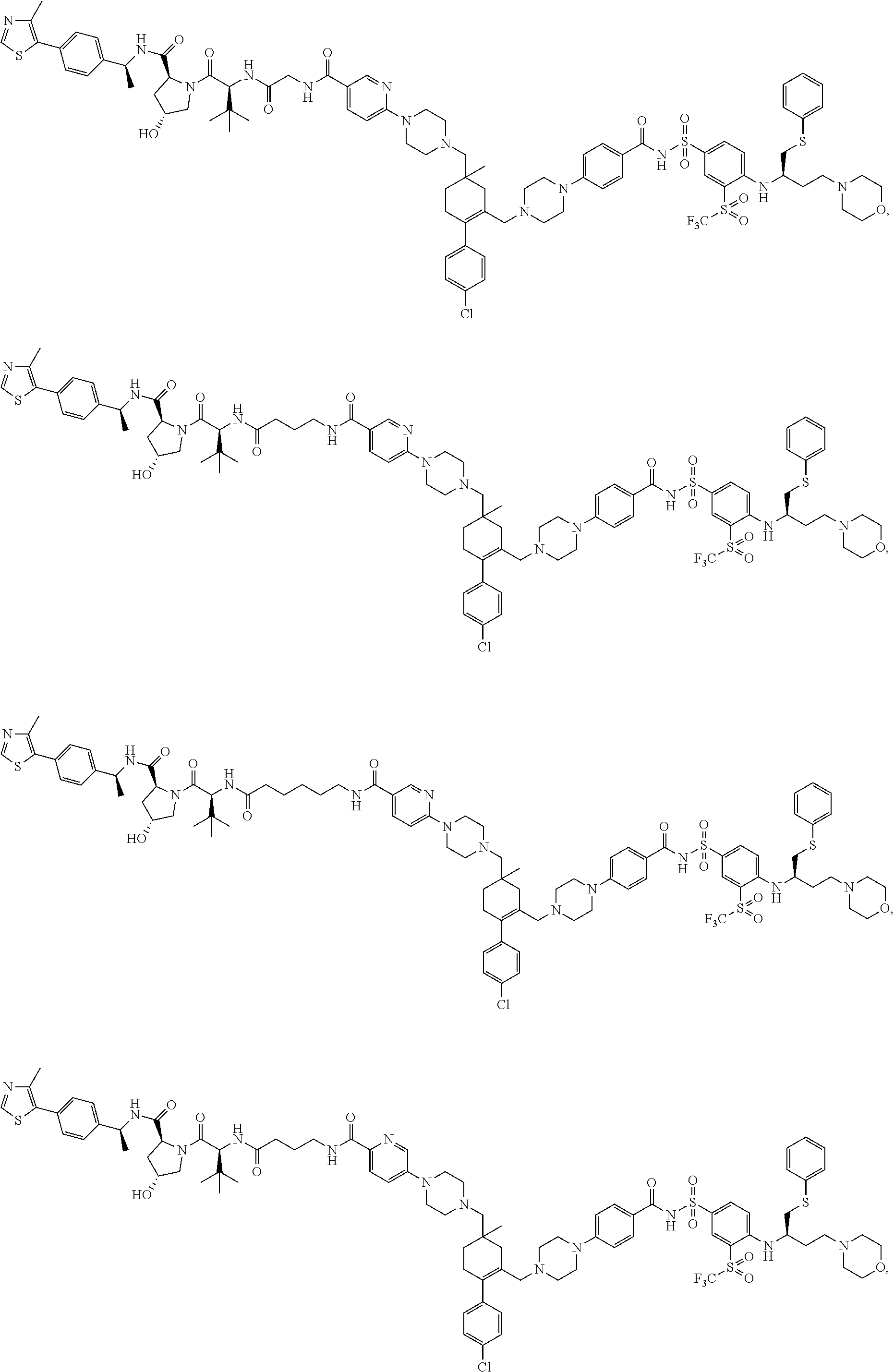 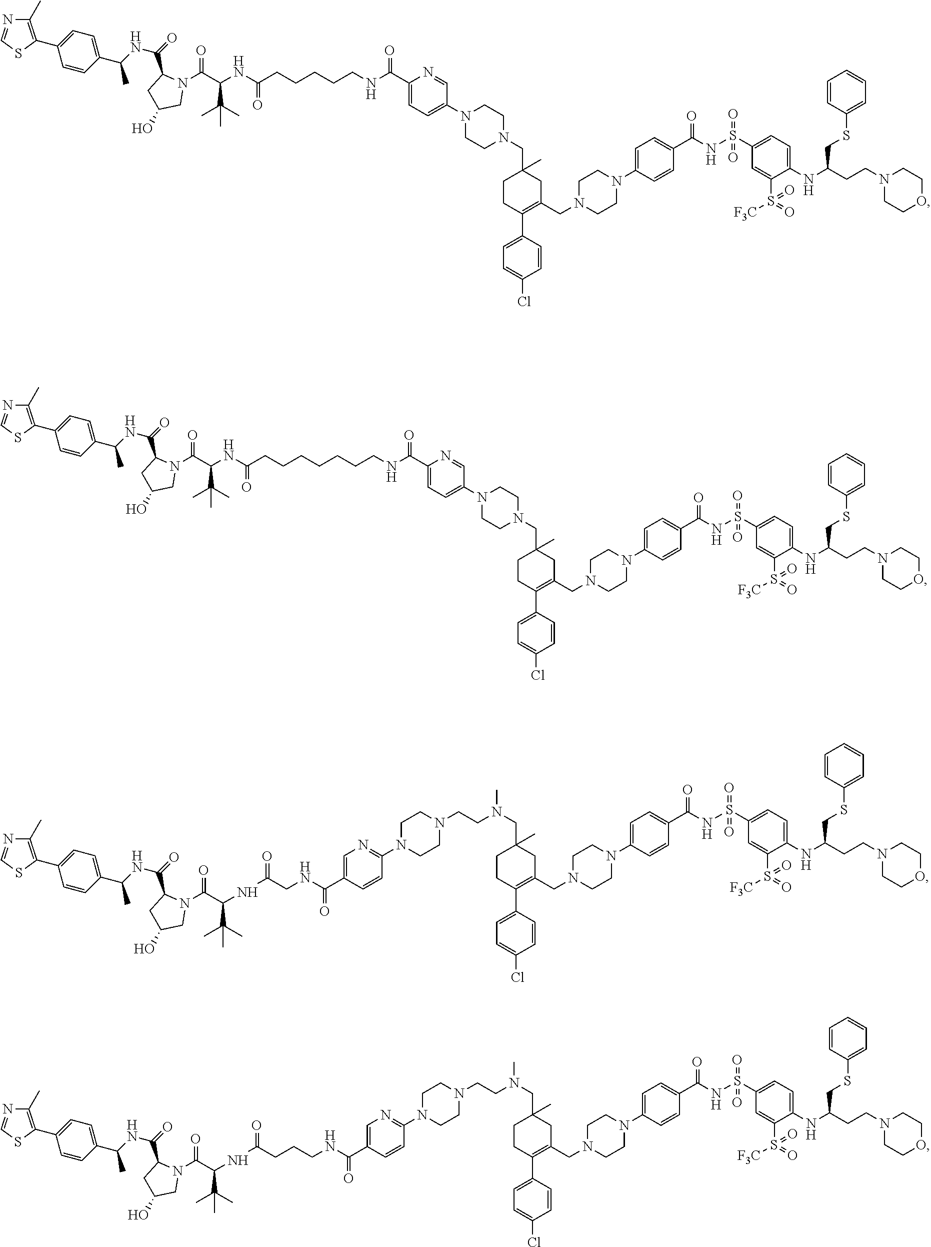 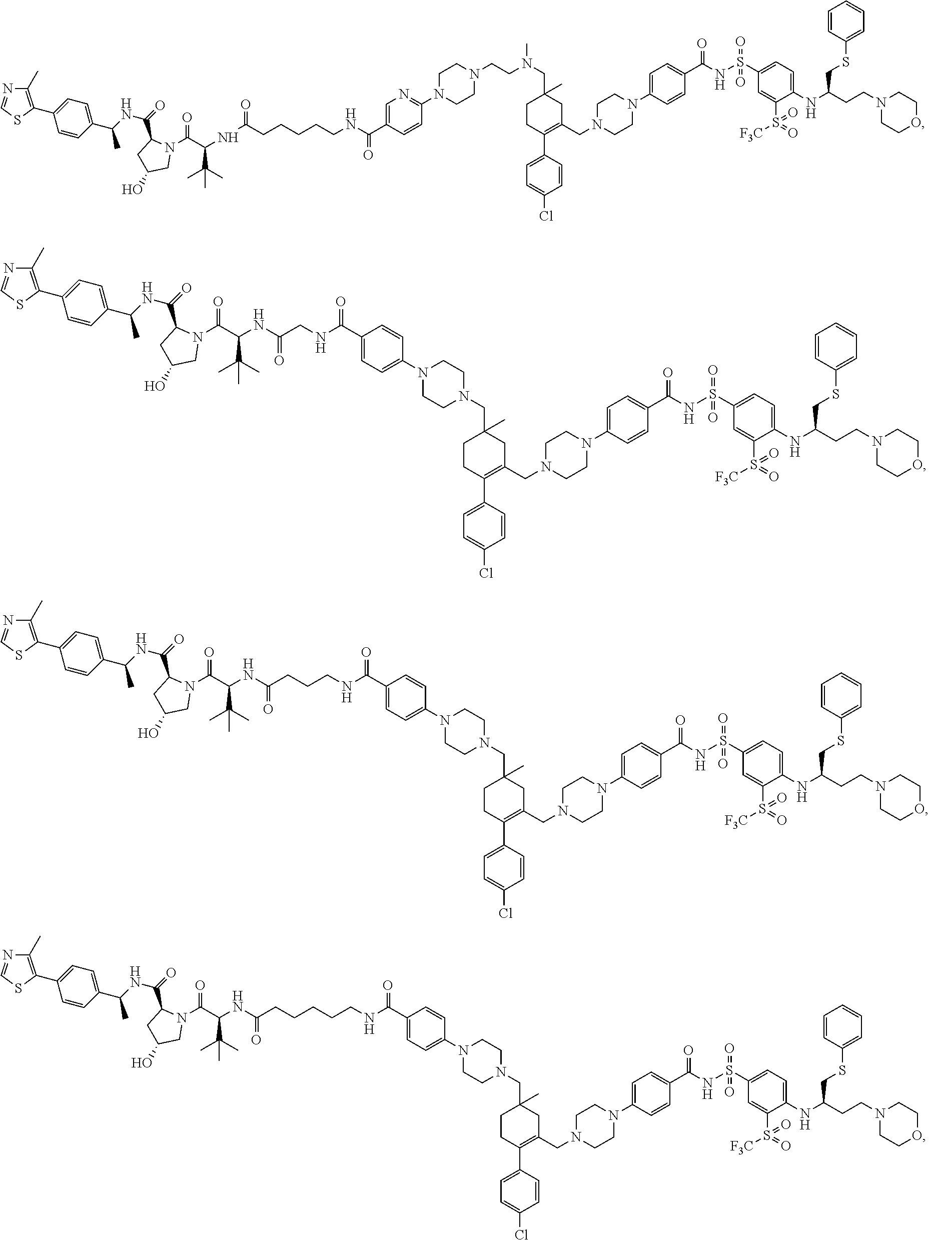 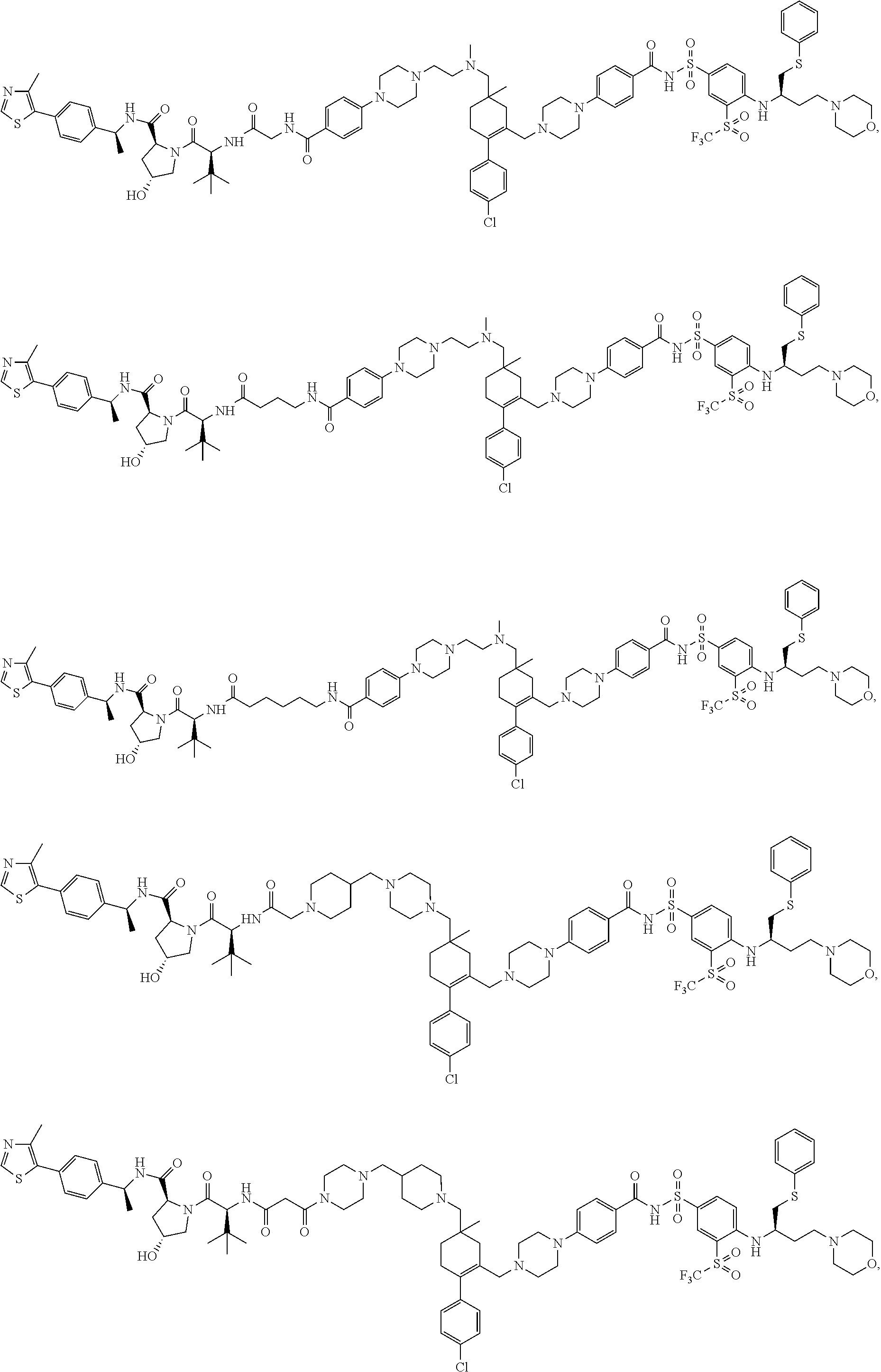 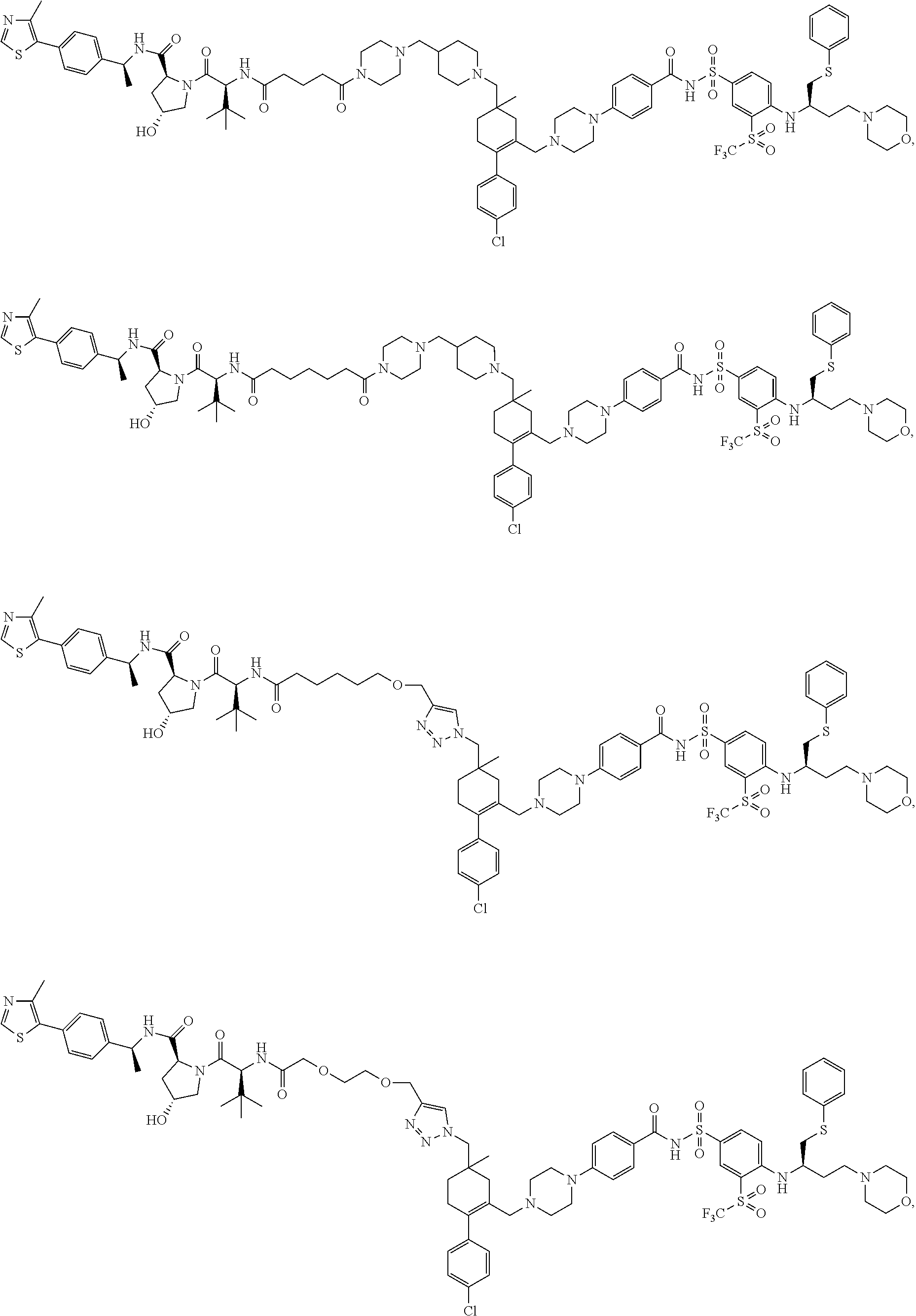 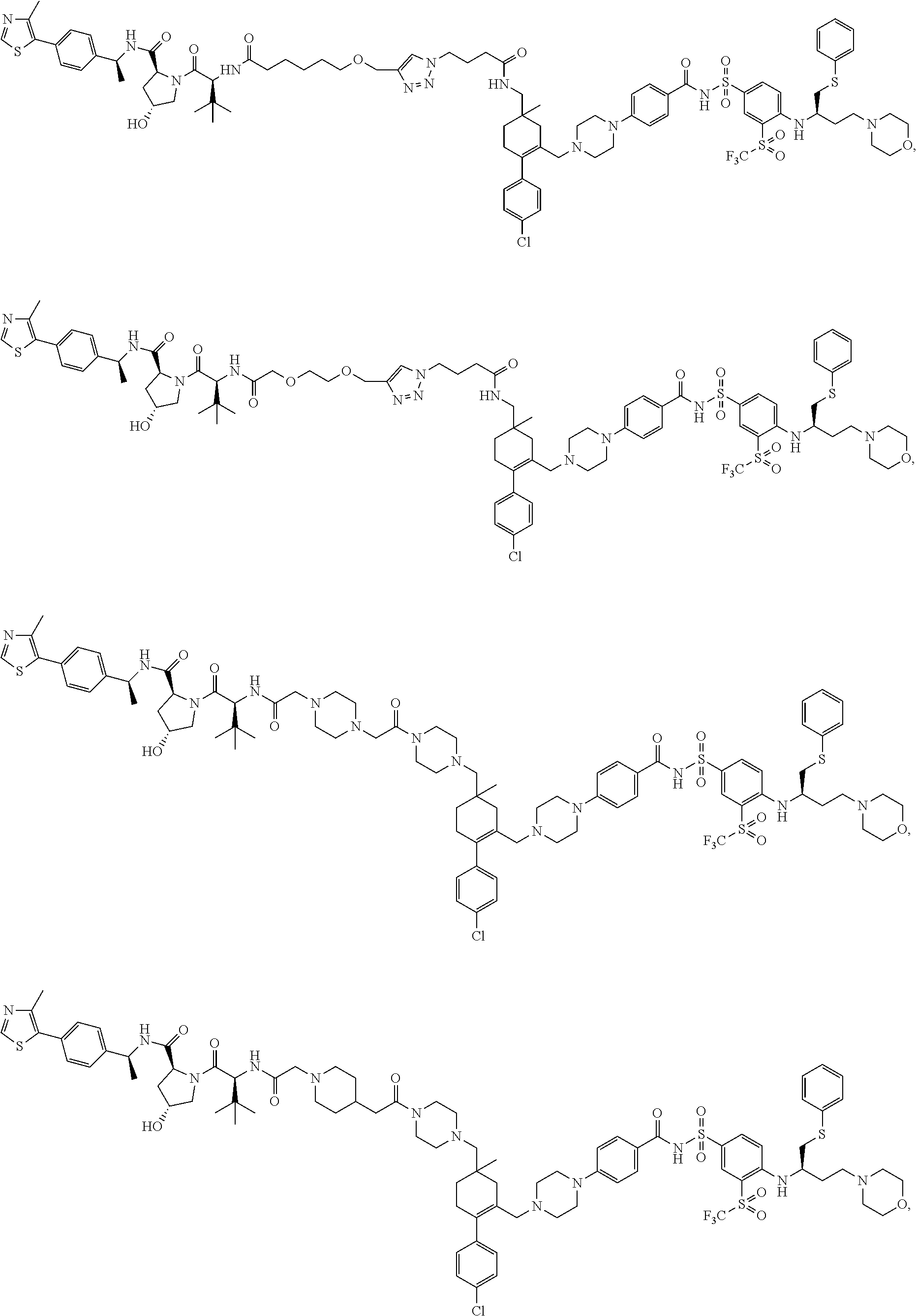 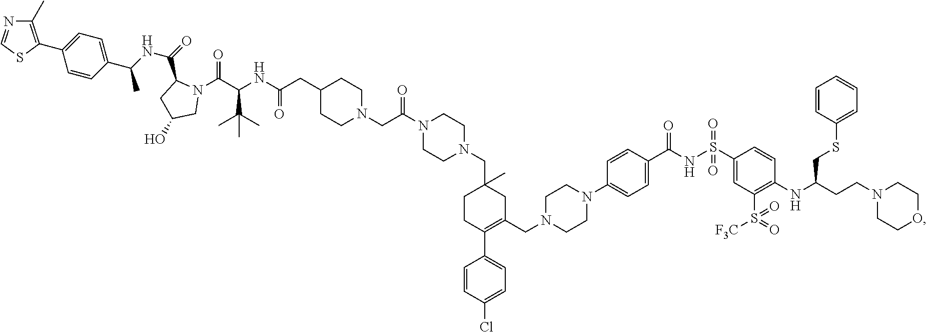 [SMILES: Cc1ncsc1-c1ccc([C@H](C)NC(=O)[C@@H]2C[C@@H](O)CN2C(=O)[C@@H](NC(=O)CC2CCN(CC(=O)N3CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC3)CC2)C(C)(C)C)cc1] 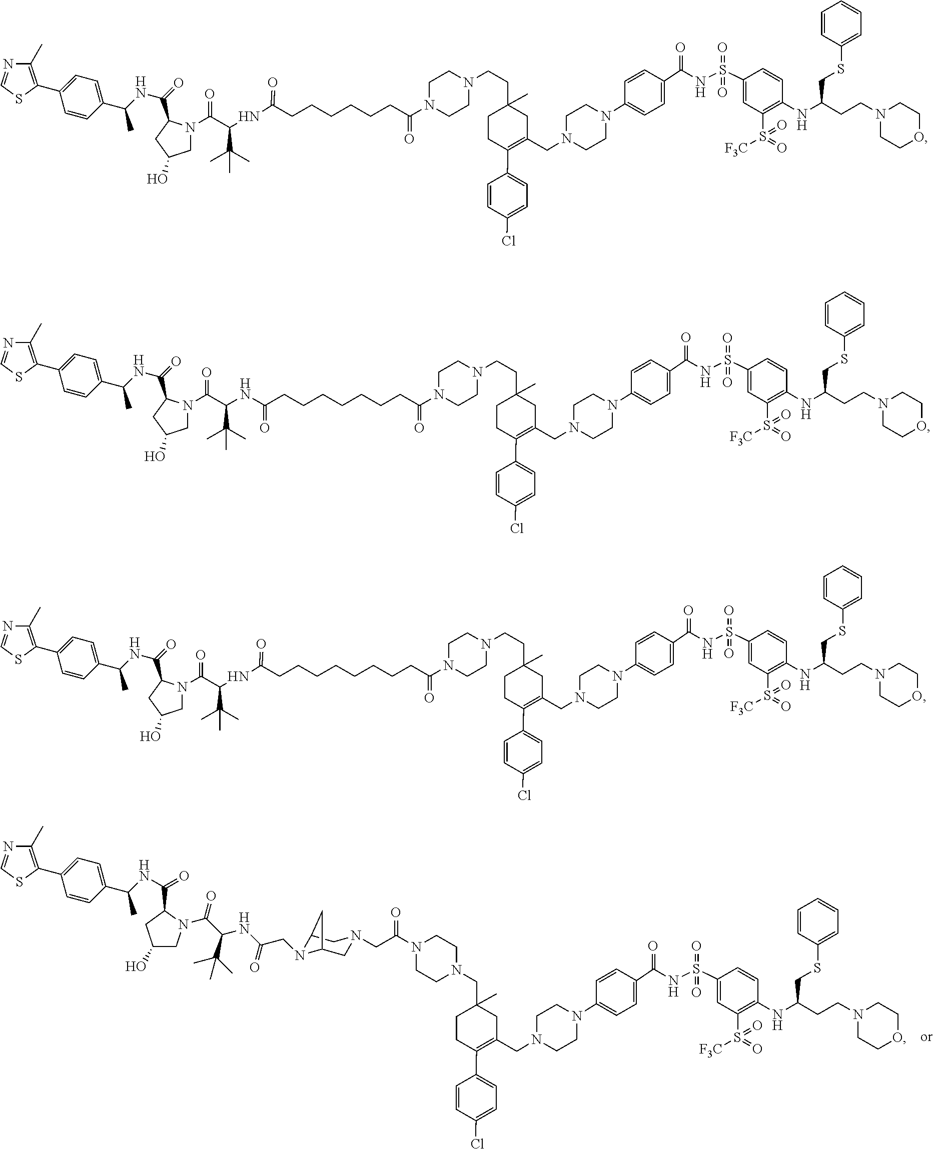 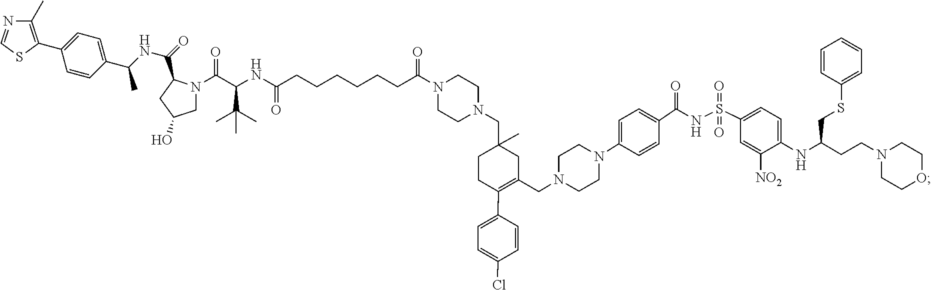 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c([N+](=O)[O-])c6)cc5)CC4)C3)CC2)C(C)(C)C)cc1]
or a pharmaceutically acceptable salt thereof.

56. The compound of claim 32, or a pharmaceutically acceptable salt thereof, wherein the compound is: 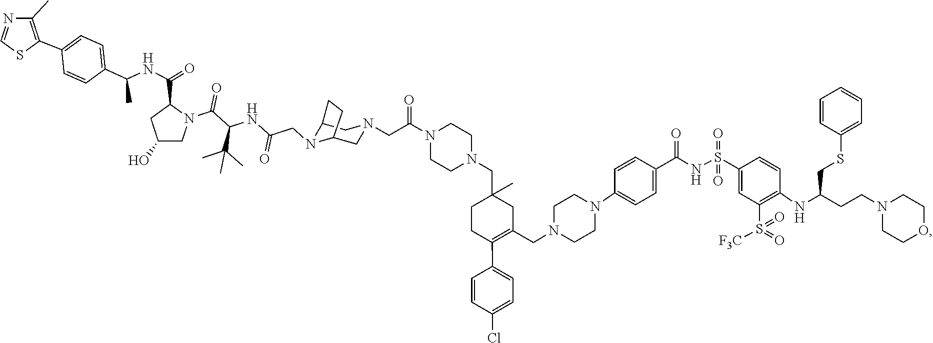 [SMILES: Cc1ncsc1-c1ccc([C@H](C)NC(=O)[C@@H]2C[C@@H](O)CN2C(=O)[C@@H](NC(=O)CN2C3CCC2CN(CC(=O)N2CCN(CC4(C)CCC(c5ccc(Cl)cc5)=C(CN5CCN(c6ccc(C(=O)NS(=O)(=O)c7ccc(N[C@H](CCN8CCOCC8)CSc8ccccc8)c(S(=O)(=O)C(F)(F)F)c7)cc6)CC5)C4)CC2)C3)C(C)(C)C)cc1]
or a pharmaceutically acceptable salt thereof.

57. A compound of the formula: 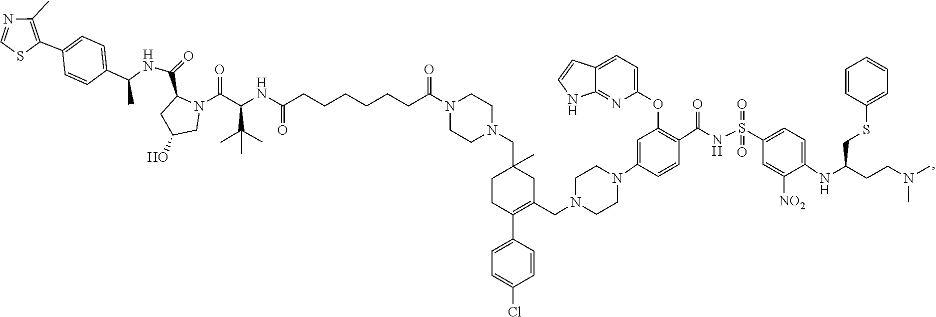 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN(C)C)CSc7ccccc7)c([N+](=O)[O-])c6)c(Oc6ccc7cc[nH]c7n6)c5)CC4)C3)CC2)C(C)(C)C)cc1]
or a pharmaceutically acceptable salt thereof.

58. A pharmaceutical composition comprising a compound of any one of claims 1-57, or a pharmaceutically acceptable salt thereof, and a pharmaceutically acceptable carrier.

59. The pharmaceutical composition of claim 58, further comprising an additional agent.

60. The pharmaceutical composition of claim 59, wherein the additional agent is an anti-cancer agent.

61. The pharmaceutical composition of claim 60, wherein the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

62. A method of degrading Bcl-2 proteins, the method comprising administering an effective amount of a compound of any one of claims 1-57, or a pharmaceutically acceptable salt thereof.

63. The method of claim 62, wherein the compound is administered in vitro.

64. The method of claim 62, wherein the compound is administered in vivo.

65. The method of claim 62, further comprising administering the compound to a subject.

66. A method of treating a disease or disorder in a subject in need thereof, the method comprising administering an effective amount of a compound of any one of claims 1-57, or a pharmaceutically acceptable salt thereof.

67. A method of treating a subject suffering from or susceptible to a disease or disorder, the method comprising administering an effective amount of a compound of any one of claims 1-57, or a pharmaceutically acceptable salt thereof.

68. The method of claim 66 or 67, wherein the disease is cancer.

69. The method of claim 68, wherein the cancer is a solid tumor.

70. The method of claim 68, wherein the cancer is chronic lymphocytic leukemia.

71. The method of claim 68, wherein the cancer is acute lymphoblastic leukemia.

72. A method of treating a Bcl-2-mediated cancer in a subject in need thereof, the method comprising administering an effective amount of a compound of any one of claims 1-57, or a pharmaceutically acceptable salt thereof, wherein the platelet toxicity of the compound is less than that of other Bcl-2 inhibitors.

73. A method of treating a subject suffering from or susceptible to a Bcl-2-mediated cancer, the method comprising administering an effective amount of a compound of any one of claims 1-57, or a pharmaceutically acceptable salt thereof, wherein the platelet toxicity of the compound is less than that of other Bcl-2 inhibitors.

74. The method of claim 72 or 73, wherein the Bcl-2-mediated cancer is chronic lymphocytic leukemia.

75. The method of claim 72 or 73, wherein the Bcl-2-mediated cancer is a solid tumor.

76. The method of claim 75, wherein the Bcl-2-mediated cancer is small cell lung cancer.

77. The method of any one of claims 72-76, wherein the other Bcl-2 inhibitor is venetoclax or ABT-263.

78. A method of treating a Bcl-2-mediated cancer in a subject in need thereof, the method comprising administering an effective amount of a compound of any one of claims 1-57, or a pharmaceutically acceptable salt thereof, wherein the ratio of human platelet toxicity (IC₅₀) to anticancer activity (IC₅₀) of the compound is greater than one.

79. A method of treating a subject suffering from or susceptible to a Bcl-2-mediated cancer, the method comprising administering an effective amount of a compound of any one of claims 1-57, or a pharmaceutically acceptable salt thereof, wherein the ratio of human platelet toxicity (IC₅₀) to anticancer activity (IC₅₀) of the compound is greater than one.

80. The method of claim 78 or 79, wherein the Bcl-2-mediated cancer is chronic lymphocytic leukemia.

81. The method of claim 78 or 79, wherein the Bcl-2-mediated cancer is a solid tumor.

82. The method of claim 81, wherein the Bcl-2-mediated cancer is small cell lung cancer.

83. The method of any one of claims 78-82, wherein the anticancer activity is measured in MOLT-4 cells.

84. The method of claim any one of claims 78-82, wherein the anticancer activity is measured in RS4 cells.

85. The method of any one of claims 78-84, wherein the anticancer activity is higher in MOLT-4 cells than in RS4 cells.

86. The method of any one of claims 78-84, wherein the anticancer activity is higher in RS4 cells than in MOLT-4 cells.

87. The method of any one of claims 78-86, wherein the ratio is greater than 2.5, greater than 5, greater than 10, greater than 20, greater than 40, greater than 60, greater than 80, greater than 100, greater than 150, greater than 200, greater than 250, greater than 300, greater than 350, greater than 400, greater than 450, or greater than 500.

88. The method of any one of claims 65-87, wherein the subject is a mammal.

89. The method of claim 88, wherein the subject is a human.
